(19) United States
(12) Patent Application Publication
(10) Pub. No.: US 2006/0134767 A1
(43) Pub. Date: Jun. 22, 2006

(54) Mitotic kinesin binding site

(76) Inventors: Carolyn A. Buser-Doepner, Harleysville, PA (US); Paul J. Coleman, Wallingford, PA (US); Christopher D. Cox, Harleysville, PA (US); Mark E Fraley, North Wales, PA (US); Robert M. Garbaccio, Lansdale, PA (US); George D. Hartman, Lansdale, PA (US); David C. Heimbrook, Warren, NJ (US); Lawrence C. Kuo, Gwynedd Valley, PA (US); Hans E. Huber, Lansdale, PA (US); Vinod V. Sardana, Lansdale, PA (US); Maricel Torrent, Lansdale, PA (US); Youwei Yan, Lansdale, PA (US)

Correspondence Address: MERCK AND CO., INC, P O BOX 2000, RAHWAY, NJ, 07065-0907, US

(21) Appl. No.: 10/520,492

(22) PCT Filed: Jul. 3, 2003
(86) PCT No.: PCT/US03/21145

§ 371 Date: Jan. 30, 2006

Related U.S. Application Data
(60) Provisional application No. 60/394,313, filed on Jul. 8, 2002.

(51) Int. Cl. C12N 9/12; G06F 19/00

(52) U.S. Cl. 435/194; 702/19

(57) ABSTRACT

The present invention is directed to the identification, characterization and three-dimensional structure of a novel ligand binding site of KSP. Binding of ligands to the novel binding site result in a conformational change in the three-dimensional structure of the protein and a modulation of the activity of KSP. This conformational change in turn results in the formation of a novel binding pocket in the KSP protein, which comprises the novel binding site of the instant invention.

### FIELD OF THE INVENTION

The present invention generally pertains to the fields of molecular biology, protein purification, protein crystallization, X-ray diffraction analysis, three-dimensional structural determination, rational drug design and molecular modeling of motor proteins, in particular—Kinesin Spindle Protein (KSP). Compositions and crystals of KSP with a KSP inhibitor bound to the protein at the novel ligand binding site identified herein are also provided. The crystallized KSP is physically analyzed by X-ray diffraction techniques. The resulting X-ray diffraction patterns are of sufficiently high resolution to be useful for determining the three-dimensional structure of inhibitor-bound KSP. Those atomic coordinates are useful in molecular modeling of related proteins and rational drug design (RDD) of mimetics and ligands for KSP and related proteins. Methods of using the structure coordinates of KSP in complex with an inhibitor for the design of pharmaceutical compositions which inhibit the biological function of KSP, particularly those biological functions mediated by molecular interactions involving KSP are also disclosed.

### BACKGROUND OF THE INVENTION

Cancer remains one of the leading causes of death in the United States. Clinically, a broad variety of medical approaches, including surgery, radiation therapy and chemotherapeutic drug therapy are currently being used in the treatment of human cancer (see the textbook CANCER: Principles & Practice of Oncology, 6th Edition, De Vita et al., eds., J. B. Lippincott Company, Philadelphia, Pa., 2001). However, it is recognized that such approaches continue to be limited by a fundamental lack of a clear understanding of the precise cellular bases of malignant transformation and neoplastic growth.

The control of cell division is one of the most basic aspects of multicellular existence. Uncontrolled cell growth and division, which produces cells that divide when they should not, produces contiguous cellular masses called tumors that are the basis for many cancers.

A common strategy for cancer therapy is the development of drugs that interrupt the cell cycle during mitosis. Compounds that perturb shortening (depolymerization) or lengthening (polymerization) cause arrest of the cell cycle in mitosis due to perturbation of the normal microtubule dynamics necessary for the chromosome movement. (Compton, D. A., et al., (1999) Science 286:913-914). A common denominator attending these compounds is that they arrest cells in mitosis by inhibiting spindle assembly (Compton, D. A., et al., (1999) Science 286:313-314). More recently, some agents such as monastrol have been implicated in inhibiting mitosis by blocking the function of essential proteins, such as mitotic proteins. (Mayer, T. U. et al., (1999) Science 286: 971-974).

The motor protein, kinesir, was discovered in 1985 in squid axoplasm. R. D. Vale et al., Identification of a Novel Force-generating Protein, Kinesin, Involved in Microtubule-based Motility, Cell 42:39-50 (1985). In the last few years, it has been discovered that kinesin is just one member of a very large family of motor proteins. E.g., S. A. Endow, The Emerging Kinesin Family of Microtubule Motor Proteins, 16 Trends Biochem. Sci. 221 (1991); L. S. B. Goldstein, The Kinesin Superfamily: Tails of Functional Redundancy, 1 Trends Cell Biol. 93 (1991); R. J. Stewart et al., Identification and Partial Characterization of Six Members of the Kinesin Superfamily in Drosophila. Proc. Nat'l Acad. Sci. USA 88:8470 (1991). Other motor proteins include dynein, e.g. M.-G. Li et al., Drosophila Cytoplasmic Dynein, a Microtubule Motor that is Asymmetrically Localized in the Oocyte, J. Cell Biol. 126:1475-1493 (1994), and myosin, e.g. T. Q. P. Uyeda et al., J. Mol. Biol. 214:699-710 (1990).

Mitotic kinesins are enzymes essential for assembly and function of the mitotic spindle, but are not generally part of other microtubule structures, such as in nerve processes. These essential microtubule-based motor proteins travel along microtubules reaching into every corner of the cell. Mitotic kinesins play essential roles during all phases of mitosis. These proteins can be conceptualized as biological machines that transduce chemical energy into mechanical forces and motion. Kinesins use the energy derived from ATP hydrolysis to power their movement unidirectionally along microtubules and to transport molecular cargo to specific destinations. During mitosis, kinesins organize microtubules into the bipolar structure that is the mitotic spindle. Kinesins mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death. It is rapidly becoming clear that mictrotubule motors play a crucial role in the functions of microtubules in mitosis.

Among the mitotic kinesins which have been identified is Kinesin Spindle Protein (KSP). KSP belongs to the BimC family of kinesins which are essentially a conserved kinesin subfamily of plus end-directed microtubule motors that assemble into bipolar homotetramers consisting of anti-parallel homodimers. Human KSP (also termed HsEg5) has been described [Blangy, et al., Cell, 83:1159-69 (1995); Whitehead, et al., Arthritis Rheum., 39:1635-42 (1996); Galgio et al., J. Cell Biol., 135:339-414 (1996); Blangy, et al., J Biol. Chem., 272:19418-24 (1997); Blangy, et al., Cell Motil Cytoskeleton, 40:174-82 (1998); Whitehead and Rattner, J. Cell Sci., 111:2551-61 (1998); Kaiser, et al., JBC 274:18925-31 (1999); GenBank accession numbers: X85137, NM004523 and U37426], and a fragment of the KSP gene (TRIP5) has been described [Lee, et al., Mol Endocrinol., 9:243-54 (1995); GenBank accession number L40372]. Xenopus KSP homologs (Eg5), as well as Drosophila K-LP61 F/KRP 130 have been reported. KSP is a mitotic kinesin protein essential for proper DNA division in cells.

During mitosis KSP associates with microtubules of the mitotic spindle. Microinjection of antibodies directed against KSP into human cells prevents spindle pole separation during prometaphase, giving rise to monopolar spindles and causing mitotic arrest and induction of programmed cell death. The current model of KSP function in mitosis envisions that KSP and related kinesins in other, non-human organisms, bundle antiparallel microtubules and slide them relative to one another, thus forcing the two spindle poles apart. KSP may also mediate anaphase B spindle elongation and focussing of microtubules at the spindle pole. The mitotic spindle has been the subject of considerable research. The study of mitotic spindle proteins, such as microtubules, has yielded anti-mitotic compounds with important applications in cancer chemotherapy. The demonstrated effectiveness of these anti-mitotic compounds in important medical and agricultural applications demonstrates the desirability of identifying and characterizing anti-mitotic compound development candidates.

Because defects in the function of KSP have been implicated in cell cycle arrest, agents and/or compounds that modulate the activity of this kinesin will find use in the treatment of hyper-proliferative cell disorders such as cancer.

Medicaments generally exhibit their biological activities through strong interactions with their respective targets. Recently, advances in protein crystallography and computational chemistry have introduced a new method of structure-based drug design into the field of drug development. X-ray crystallography (crystallography) is an established, well-studied technique that provides what can be best described as a three-dimensional picture of what a molecule looks like in a crystal. Scientists have used crystallography to solve the crystal structures for many biologically important molecules. Many classes of biomolecules can be studied by crystallography, including, but not limited to, proteins, DNA, RNA and viruses.

Crystallography has been used extensively to view ligand-protein complexes for structure-based drug design. To view such complexes, known ligands are usually soaked into the target molecule crystal, followed by crystallography of the complex. Sometimes, it is necessary to co-crystallize the ligands with the target molecule to obtain a suitable crystal.

Given a “picture” of a target biomolecule or a ligand-protein complex, scientists can look for pockets or receptors where biological activity can take place. Thereafter, scientists can experimentally or computationally design high-affinity ligands (or drugs) for the protein/receptors. Computational methods have alternatively been used to screen for the binding of small molecules. This approach is also useful for developing new anti-mitotic agents.

Recently, independent efforts have confirmed the role of mitotic kinesins as critical mediators of microtubule organization during mitosis. It is postulated that blocking the biological function of motor proteins, e.g., human KSP, will lead to cell cycle arrest. While the binary structure of KSP complexed with ADP has been published, (Turner et al., Journal of Biological Chemistry, 276; 25496-25502 (2001), no ternary structure of KSP complexed with a modulator, e.g., inhibitor, has heretofore been published. Consequently, until the present invention, which details the structural coordinates of human KSP with various ligands, albeit inhibitors, the identity and characterization of the novel binding site detailed herein was heretofore never available for rational drug design. As such, drug discovery efforts directed towards the KSP protein have been hampered by the lack of structural information about this protein and its complex with a ligand, e.g., monastrol. Such structural information would provide valuable information in discovery of anti-mitotic agents.

The inventors provide herein crystals of KSP, complexed with a ligand, containing a novel, induced-fit binding site and have determined its three-dimensional structure. With this information, it is now possible, for the first time, to rationally design inhibitors of KSP, which can function as anti-mitotic agents, e.g. compounds which inhibit spindle pole separation during mitosis, thereby effectively inducing cell cycle arrest. It is believed that no one has heretofore reported determining the three-dimensional structure of the binding site identified herein.

Advantageous therapeutic embodiments would therefore comprise therapeutic and/or diagnostic agents based on or derived from the three-dimensional crystal structure of KSP including its novel binding site identified herein that have one or more than one of the functional activities of KSP. Additional therapeutic embodiments would comprise therapeutic and/or diagnostic agents based on or derived from molecular modeling of other members of the BimC protein family using the three-dimensional crystal structure of KSP and its binding site provided herein.

In accordance therewith, the novel-binding site disclosed herein is considered a potential target for anti-mitotic agents. In addition, the invention provides a process for creation of ligand candidate structures by means of a computer, using the structural coordinates of KSP's binding site provided herein. Furthermore, the information provided herein will enable one to search for ligand structures from a three-dimensional structure database containing known compounds.

### SUMMARY OF THE INVENTION

The present invention is directed to the identification, characterization and three-dimensional structure of a novel ligand binding site of KSP. Binding of ligands to the novel binding site result in a conformational change in the three-dimensional structure of the protein and a modulation of the activity of KSP. This conformational change in turn results in the formation of a novel binding pocket in the KSP protein, which comprises the novel binding site of the instant invention. It has been further discovered that the formation of the novel binding pocket is facilitated by the concurrent binding of a nucleotide substrate or substrates to the protein. Moreover, the instant invention provides an attractive target for the rational design of potent and selective inhibitors of KSP identified by the methods of the invention, particularly new lead compounds useful in treating hyper-proliferative and KSP-dependent disorders.

### BRIEF DESCRIPTION OF THE DRAWINGS

FIG. 1 An X-ray oscillation diffraction picture from a crystal of KSP in complex with (+)-monastrol and ADP (Compound 5-2b).

FIG. 2 The KSP-ADP-(+)-monastrol complex as shown in a ribbon presentation. The structure of the KSP-ADP-(+)-monastrol (Compound 5-2b) complex is shown in a ribbon representation. The bound conformations of ADP and Compound 5-2b are also given together with their respective electron density. The location of Compound 5-2b, the active isomer of monastrol, is seen at a novel induced-fit site, some 12 Å distal from the nucleotide-binding site and catalytic center of the enzyme.

FIG. 3 (+)-Monastrol binding between helix-α2 and helix-α3. (+)-monastrol (Compound 5-2b) is seen to bind in between (the insertion loop of) helix-α2 and helix-α3 (which is immediately preceding the ‘Switch 1’ typically seen in all kinesins). Also shown are the side-chains of Arg119, Tyr211 and Trp127. The Arg119 and Tyr211 residues move upward and outward, yielding space to accommodate the binding of the inhibitor. At the same time, the insertion loop of helix-α2 relocates its main-chain location with a downward shift of ˜8 Å; the side-chain of its Trp127 as a result swings inward by ˜10 Å, capping the entrance of the induced-fit cavity together with the side-chains of Arg119 and Tyr211. Lining the newly formed pocket and surrounding the inhibitor are residues 115-119, 127, 130, 132-134, 136, 137, 160, 211, 214, 215, 217, 218, 221 and 239.

FIG. 4 Comparison between the binary and ternary structure shown in ribbon presentation. The conformational alteration observed for the kinesin structure upon Compound 5-2b binding to the ADP-binary complex is not limited to the immediate vicinity of the inhibitor. Rearrangements of protein moieties are spread throughout the enzyme upon (+)-monastrol binding, including the switch I, switch II and neck linker region, with the exception that the nucleotide binding site of the protein as well as its β-sheet structure remaining basically unchanged.

FIG. 5 Conformational alteration of KSP structure upon ligand binding shown in ribbon presentation. In the Switch I area of KSP, as circled, the main-chain re-orients its geometry significantly on both ends of Ala230. Although the helicity of the Switch I region is unchanged, the pitch at the C-terminal end of helix-α3 is increased in the ternary complex from that in the binary complex.

FIG. 6 Conformational alteration of KSP structure upon ligand binding shown in ribbon presentation. In the Switch II region of KSP, which is located on the opposite side of the binding site, as circled, the C-terminal end of helix-α-4 is repositioned significantly. The tip of the helix, in the Switch II region of KSP, near Arg305 is moved by ˜6 Å in the ternary complex from its location in the binary complex.

FIG. 7 Conformational alteration of KSP structure upon ligand binding shown in ribbon presentation. In the neck-linker region of KSP, which is the C-terminal portion of the protein construct, the residues beginning from Lys357 to Phe362 swing by almost 180° in the ternary complex from its position in the ADP binary complex. Although residues 363-368 are present in the protein, they are disordered in the crystal and hence offer no electron density. The neck-linker region of KSP is circled. A close-up view is depicted, comparing the neck-linker region in the ternary complex to that in the binary complex.

FIG. 8 Conformational alteration of KSP structure upon ligand binding. A close-up view comparing the nucleotide-binding site in the binary and ternary complexes of KSP is shown. Within experimental errors, most of the backbone and side-chains for the two complexes in this region of the protein can be super-positioned.

FIG. 9 Motor Domain of Human KSP, Amino Acids 1-368.

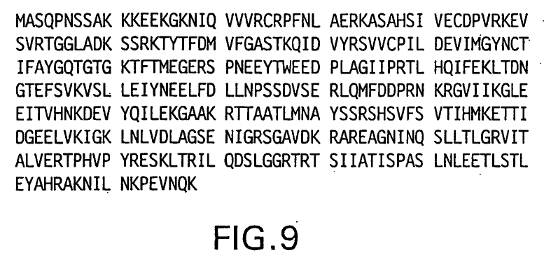

FIG. 10 Binding Pocket of human KSP.

FIG. 11 KSP/Compound 5-2b fluorescence data. Compound 5-2b demonstrates a dose dependent decrease on the fluorescence of Trp127 in the presence of ADP or AMPPNP. These data indicate that the fluorescence assay is useful to measure potential KSP inhibitors. In the absence of the nucleotide, 5-2b does not cause a decrease on Trp127 fluorescence, suggesting the inability of 5-2b to bind to KSP in the absence of the nucleotide.

FIG. 12 KSP/Compound 8-1 fluorescence data. Compound 8-1 demonstrates a dose dependent decrease on the fluorescence of Trp127 in the presence of ADP or AMPPNP. These data indicate that the fluorescence assay is useful to measure potential KSP inhibitors. In the absence of the nucleotide, 8-1 does not cause a decrease on Trp127 fluorescence, suggesting the inability of 8-1 to bind to KSP in the absence of the nucleotide.

FIG. 13 KSP/Compound 1-7 fluorescence data. Compound 1-7 demonstrates a dose dependent decrease on the fluorescence of Trp127 in the presence of ADP or AMPPNP. These data indicate that the fluorescence assay is useful to measure potential KSP inhibitors. In the absence of the nucleotide, 1-7 does not cause a decrease on Trp127 fluorescence, suggesting the inability of 1-7 to bind to KSP in the absence of the nucleotide.

FIGS. 14A and 14B KSP Inhibitor Pharmacophore Models. The two pharmacophore models derived from analysis and further computational processing of the crystallized complex are illustrated. Spheres represent a center of a hydrophobic group and boxes represent either a hydrogen bond acceptor (HA) or hydrogen bond donor (HD). All distances are in Å.

FIG. 15 KSP Inhibitor Pharmacophore Models in KSP Binding Site. A schematic view of the two pharmacophore models superimposed and mapped onto the ligand binding site of KSP defined, in part, by the amino acids of FIG. 10. Only relevant KSP protein residues are shown.

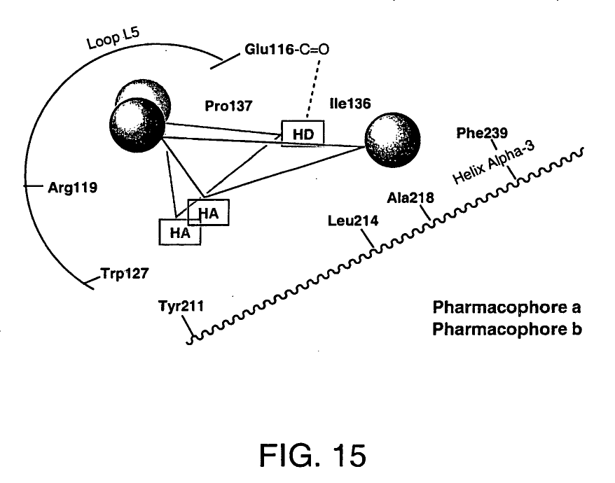

FIG. 16 KSP Inhibitor Pharmacophore Model. A pharmacophore model derived from analysis and further computational processing of a crystallized complex is illustrated. Spheres represent a center of a hydrophobic group and boxes represent either a hydrogen bond acceptor (HA).

TABLE 1 KSP motor domain/Compound 5-2b X-ray coordinates.

TABLE 2 KSP motor domain/Compound 1-7 X-ray coordinates.

TABLE 3 KSP motor domain/Compound 2-7 X-ray coordinates.

TABLE 4 KSP motor domain/Compound 4-2a X-ray coordinates.

TABLE 5 Novel KSP ligand binding site/Compound 5-2b X-ray coordinates.

### DETAILED DESCRIPTION OF THE INVENTION

“Conservative substitutions” are those amino acid substitutions which are functionally equivalent to the substituted amino acid residue, either by way of having similar polarity, steric arrangement, or by belonging to the same class as the substituted residue (e.g., hydrophobic, acidic or basic), and includes substitutions having an inconsequential effect on the three-dimensional structure of KSP with respect to the use of said structure for the identification and design of KSP or KSP complex inhibitors, for molecular replacement analyses and/or for homology modeling.

Amino acid sequence “similarity” is a measure of the degree to which aligned amino acid sequences possess identical amino acids or conservative amino acid substitutions at corresponding-positions.

A “fragment” of KSP is meant to refer to a protein molecule which contains a portion of the complete amino acid sequence of the wild type or reference protein.

As used herein, a “variant” of a KSP protein refers to a polypeptide having an amino acid sequence with one or more amino acid substitutions, insertions, and/or deletions compared to the sequence of the invention receptor protein. Generally, differences are limited so that the sequences of the reference (native or wild type KSP) and the variant are closely similar overall, and in many regions, identical. Such variants are generally biologically active and necessarily have less than 100% sequence identity with the polypeptide of interest.

Preferably, the biologically active variant KSP has an amino acid sequence sharing at least about 80% amino acid sequence identity with the reference KSP, preferably at least about 85%, more preferably at least about 90%, and most preferably at least about 95%. Amino-acid substitutions are preferably substitutions of single amino-acid residues. Preferably, such polypeptides also possess characteristic structural features and biological activity of a native KSP polypeptide. For example, variants of KSP are characterized as containing key functional residues that participate in ligand binding. These polypeptide fragments, in turn, have been derivatized by methods akin to traditional drug development. Preferred polypeptides and polynucleotides of the present invention are expected to have, inter alia, similar biological functions/properties to their homologous polypeptides and polynucleotides. Furthermore, preferred polypeptides and polynucleotides of the present invention have at least one GPR25 activity.

Sequence similarity or percent similarity can be determined, for example, by comparing sequence information using sequence analysis software such as the GAP computer program, version 6.0, available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math. 2:482, 1981).

As used herein, a “binding site” refers to a region of a molecule or molecular complex that, as a result of its shape and charge potential, favorably interacts or associates with another agent (including, without limitation, a protein, polypeptide, peptide, nucleic acid, including DNA or RNA, molecule, compound, antibody or drug) via various covalent and/or non-covalent binding forces.

The terms “ligand binding site” and “binding site” are used interchangeably and refer to a region of a human KSP resulting from the complex of a ligand with KSP. It is believed that this ligand binding site, as a result of its shape and charge potential, favorably interacts or associates with a ligand or binding partner, which is preferably an inhibitor of KSP function. The binding of the ligand to this binding site induces global conformational changes to the KSP protein, thereby potentially modulating the mitotic activity of the protein and thereby inhibiting cell division and facilitating cell cycle arrest. A ligand binding site according to the present invention may include, for example, the actual site of any one of the herein disclosed compounds binding with KSP, as well as any other moiety—chemical or biological—which preferably inhibits the activities of KSP by binding to the ligand binding site disclosed herein.

As used herein, the terms “bind” and “binding” when used to describe the interaction of a ligand with a binding site or a group of amino acids means that the binding site or group of amino acids are capable of forming a covalent or non-covalent bond or bonds with the ligand. Preferably, the binding between the ligand and the binding site or amino acid(s) is non-covalent. Such a non-covalent bond includes a hydrogen bond, an electrostatic bond, a van der Waals bond or the like. The binding of the ligand to the binding site may also be characterized by the ability of the ligand to co-crystallize with KSP within the novel binding pocket of the instant invention. It is further understood that the use of the terms “bind” and “binding” when referring to the interaction of a ligand with the novel binding site of the instant invention includes the covalent or non-covalent interactions of the ligand with all or some of the amino acid residues comprising the binding site.

A “KSP complex” refers to a co-complex of a molecule/complex comprising the KSP in bound association with a ligand either by covalent or non-covalent binding forces at the binding site disclosed herein. A non-limiting example of a KSP complex includes KSP-(+)-monastrol, or KSP bound to any one of the compounds listed herein.

The present invention relates to the three-dimensional structure of ligand bound-KSP or of a KSP analogue, and more specifically, to the structure of KSP's binding site as determined using X-ray crystallography and various computer modeling techniques. The coordinates of KSP bound to ADP and one of the ligand compounds described herein as shown in Tables 1-4 (relating to the entire motor domain), are useful for a number of applications, including, but not limited to, the characterization of a three-dimensional structure of KSP including its novel binding site, as well as the visualization, identification and characterization of a KSP ligand binding site. The ligand binding site structure(s) may then be used to predict the orientation and binding affinity of a designed or selected inhibitor of KSP, a KSP analogue or of a KSP complex. In general, KSP structures referred to herein are the KSP-ligand bound conformation of KSP. As an example, when referring to an antibody specific for the KSP of the invention, it means an antibody having an affinity for the KSP-ligand bound conformation disclosed herein.

In particular, the invention is drawn to the three-dimensional structure of a ligand bound KSP e.g., when bound to a ligand, preferably an inhibitor.

The amino acid sequence of the motor domain of human KSP is depicted in SEQ ID NO:1. These amino acids correspond to residues 1-368 of the native protein. Another aspect of the invention is a substantially pure isolated amino acid of the amino acid sequence set forth in SEQ ID NO:1. Another aspect of the invention is a variant of that isolated amino acid. Preferably the variant of the amino acid of SEQ ID NO:1 comprises one or more amino acid substitution(s) or deletion(s) of one or more of the amino acids that form the novel binding pocket of the instant invention. More preferably the variant of the amino acid of SEQ ID NO:1 comprises an amino acid substitution of one of the amino acids which form the novel binding pocket of the instant invention.

Another aspect of the invention is an isolated variant of KSP wherein the variant comprises one or more amino acid substitution(s) or deletion(s) of one or more of the amino acids that form the novel binding pocket of the instant invention. More preferably the variant of KSP comprises an amino acid substitution of one of the amino acids which form the novel binding pocket of the instant invention.

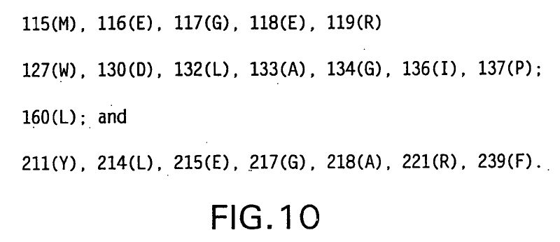

The KSP of the invention preferably comprises a ligand binding site characterized by the amino acid residues as set forth in FIG. 10 or the relative structural coordinates of those amino acid residues according to Tables 1-4 ±a root mean square deviation from the conserved backbone atoms of said amino acids of not more than about 2.0 Å (or more preferably, not more than about 1.0 Å, and most preferably, not more than about 0.5 Å). It is understood that the amino acids listed above represent the residues defining the novel binding pocket formed upon the complexation of a ligand of the invention with KSP. It is further understood that specific binding interactions between the listed residues may or may not occur based on the size of the ligand and structure of the ligand. It is also understood that the computational length of the allowable van der Waals interactions is also a factor when determining whether an amino acid residue binds to a ligand. It is therefore understood that the binding of a ligand of the instant invention may take place between those residues listed in FIG. 10 or a subset thereof.

It has been surprisingly discovered that compounds previously disclosed as kinesin inhibitors, and other recently identified inhibitors of KSP, bind to the KSP protein at the novel binding site described herein. In particular, (+)-monastrol (Compound 5-2b), a compound previously described as inhibiting KSP kinesin activity (see Mayer, T. U. et al. Science 286:971 (1999)) has been found to be a ligand of the novel binding site of the invention. Inhibitors of KSP have also been disclosed in pending U.S. provisional applications Ser. Nos. 60/344,453 (Case 20990PV), 60/338,383 (Case 20995PV), 60/338,380 (Case 20996PV), 60/338,779 (Case 20997PV), 60/338,344 (Case 20998PV), 60/338,379 (Case 20999PV), 60/362,922 (Case 21047PV), 60/383,449 (Case 21018PV), 60/383,478 (Case 21060PV), 60/388,621 (Case 21114PV, filed Jun. 14, 2002) and 60/388,828 (Case 21119PV, filed Jun. 14, 2002). Additionally, inhibitors of KSP kinesin activity are described in PCT Publications WO 01/30768 and WO 01/98278.

The 3-dimensional structure of KSP, bound with Mg⁺⁺-ADP and Compound 5-2b, was determined at 2.5 Å resolution. Compound 5-2b was found to bind to KSP via an induced-fit some 12 Å away from the catalytic center of the enzyme, resulting in the creation of a previously unknown binding pocket that is non-existent in the absence of Compound 5-2b (or the other ligands described herein). The binding of Compound 5-2b also introduced significant alteration to the structural conformation in other regions of the KSP motor protein, with the interesting exception that the nucleotide-binding pocket was virtually unaltered from that seen in the ADP binary complex. An analysis of the temperature-factor distribution in the ADP binary and ADP/5-2b ternary complexes of KSP revealed that the protein region surrounding the induced-fit binding pocket of 5-2b became highly rigid upon 5-2b binding.

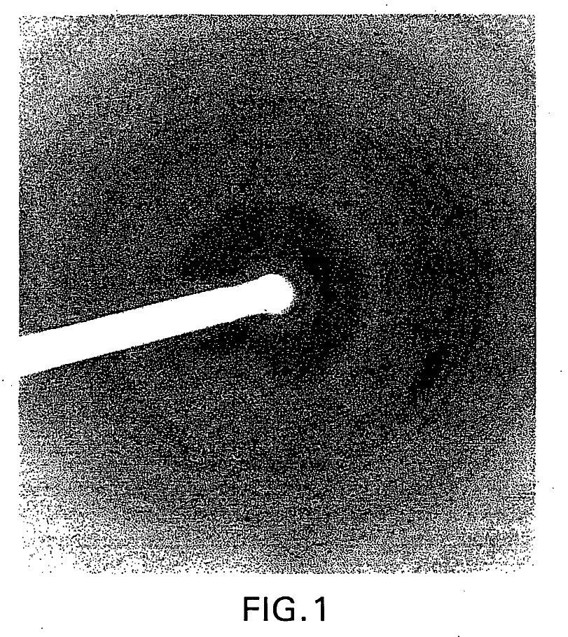

Using the seeding method, high quality single crystals were obtained for KSP prepared in the presence of ADP and 5-2b. A diffraction data set to 2.5 Å resolution was collected and processed in the orthorhombic P2₁,2₁2₁ space group. The R_{sym} was 0.084 and the data completeness was 99%. The cell dimensions were 69.5 Å, 79.5 Å and 159.0 Å. An oscillation X-ray diffraction picture of a KSP crystal is given in FIG. 1.

The 3-dimensional, tertiary structure of KSP, bound with Mg⁺⁺-ADP and 5-2b, was determined at 2.5 Å resolution with use of phases derived from a combination of molecular replacement, extensive manual rebuilding, and dynamic refinement. Two identical protein complexes were found in the asymmetric unit of the crystal and were related by a local, non-crystallographic 2-fold axis. For each, the electron density of the protein as well as those of the ligands (ADP, Mg⁺⁺, and 5-2b) was all well defined. 5-2b was seen to be of the S handedness. Residues 2-17, 272-286, and 363-368 were disordered and showed no electron densities (The N-terminal Met1 residue was processed upon expression).

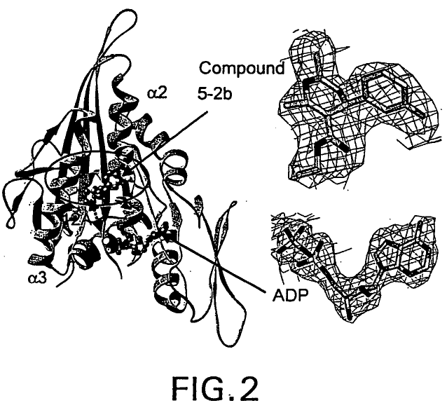

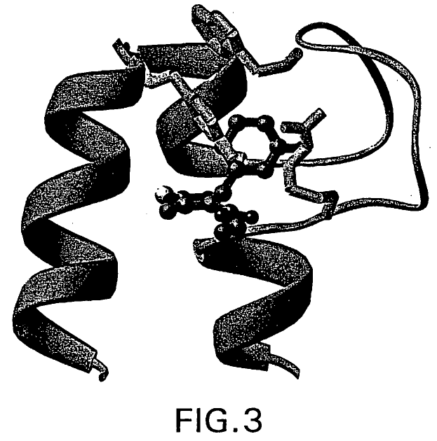

The structure of the KSP/ADP/Compound 5-2b complex is shown (FIG. 2) in a ribbon representation. The bound conformations of ADP and 5-2b are also given together with their respective electron density. The location of 5-2b is seen at a novel induced-fit site, some 12 Å distal from the nucleotide-binding site and catalytic center of the enzyme. An enlarged section of this region is shown in FIG. 3, together with 5-2b.

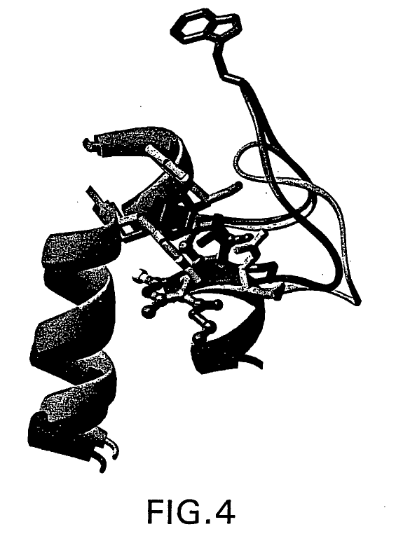

In FIG. 3 the Compound 5-2b is seen to bind in between (the insertion loop of) helix-α2 and helix-α3 (which is immediately preceding the ‘Switch 1’ typically seen in all kinesins). Also shown are the side-chains of Arg119, Tyr211 and Trp127. The Arg119 and Tyr211 residues move upward and outward, yielding space to accommodate the binding of the inhibitor. At the same time, the insertion loop of helix-α2 relocates its main-chain location with a downward shift of ˜8 Å; the side-chain of its Trp127 as a result swings inward by ˜10 Å, capping the entrance of the induced-fit cavity together with the side-chains of Arg119 and Tyr211. Lining the newly formed pocket and surrounding the inhibitor are the amino acid residues listed in FIG. 10. A comparison of this region in the binary and ternary complex is given in FIG. 4.

The binding pocket of Compound 5-2b is novel and not previously known, insofar that this binding site does not exist until an inhibitor binds. Hence, this pocket is “induced-fit” by a ligand such as Compound 5-2b. This allosteric binding pocket, located away from the nucleotide-binding site of the motor protein, is not restricted to Compound 5-2b, but is also observed upon the crystal structure determination of complexes of KSP with other compounds of diverse chemical structure that are inhibitors of KSP activity. These results have a profound impact on the design of non-active-site directing inhibitors of KSP.

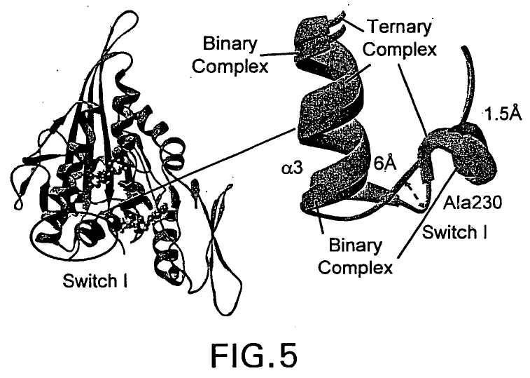

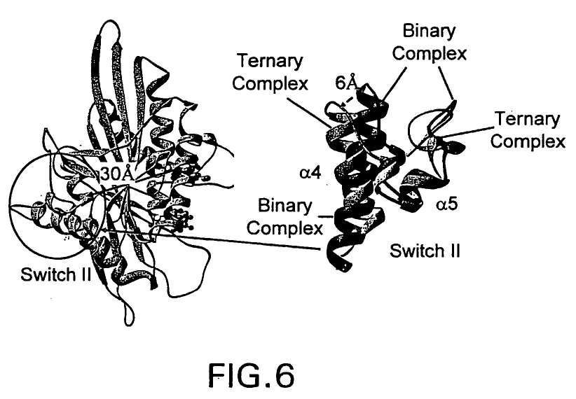

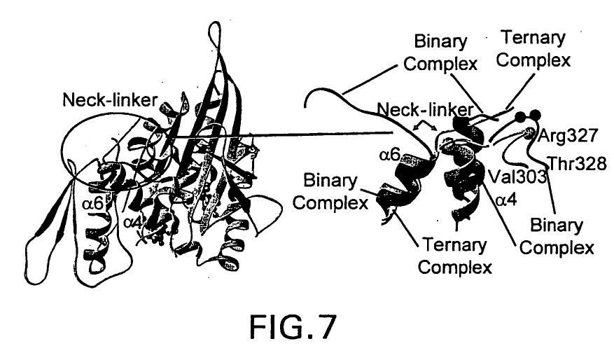

In a further embodiment of the invention is a method of causing a conformational alteration in the structure of KSP by exposing the KSP to a ligand of the novel ligand binding site of the instant invention. The conformational alteration observed for the kinesin structure upon Compound 5-2b binding (and the binding of other compounds) to the ADP-KSP binary complex is not limited to the immediate vicinity of the inhibitor. Rearrangements of protein moieties are spread throughout the enzyme upon 5-2b binding, with the exception that the nucleotide binding site of the protein as well as its β-sheet structure remain basically unchanged. Among the changes away from the induced-fit pocket, three are noteworthy:
1. In the Switch I area of KSP, as circled in FIG. 5 and in a close-up view, the main-chain re-orients its geometry significantly on both ends of Ala230. It can be seen that although the helicity of the Switch I region is unchanged, the pitch at the C-terminal end of helix-α3 is increased in the ternary complex from that in the binary complex.
2. In the Switch II region of KSP, which is located on the opposite side of the 5-2b binding site as circled in FIG. 6 and in a close-up view, the C-terminal end of helix-α4 is repositioned significantly. The tip of this helix near Arg305 is moved by ˜6 Å in the ternary complex from its location in the binary complex.
3. In the neck-linker region of KSP, which is the C-terminal portion of our protein construct, the residues beginning from Lys357 to Phe362 swing by almost 180° in the ternary complex from its position in the ADP binary complex. Although residues 363-368 are present in our protein, they are disordered in the crystal and hence offer no electron density. The neck-linker region of KSP is circled in FIG. 7. A close-up view is depicted comparing this region in the ternary complex to that in the binary complex.

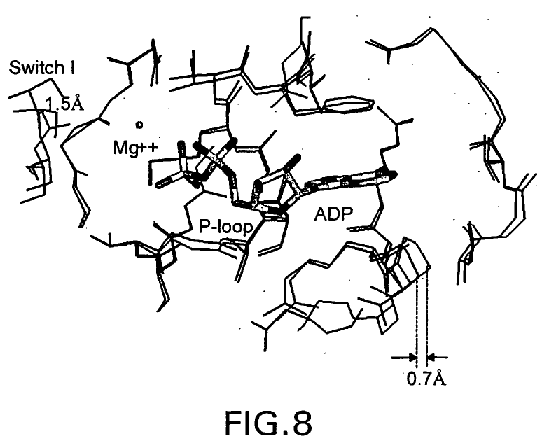

In addition to these changes, there are other smaller regional repositionings of main-chains and side-chains of the protein. Most interestingly, the nucleotide-binding site of the motor protein, where ATP hydrolysis occurs, is basically unaltered upon 5-2b binding. A close-up view comparing this site in the binary and ternary complexes of KSP is shown in FIG. 8. Within experimental errors, most of the backbone and side-chains for the two complexes in this region of the protein can be super-imposed.

The effect of overall conformational changes induced by Compound 5-2b could also be examined by comparing the distribution of temperature factors.

High quality single crystals were also obtained for other compounds that are inhibitors of KSP. 3-Dimensional structure determined at 2.5 Å with those crystals demonstrated that the other inhibitor compounds also induce-fit into the protein in the same manner as compound 5-2b.

Consequently, an embodiment of the invention provides protein crystals of KSP complexed with a ligand bound to the ligand binding site disclosed herein and methods for making KSP or a KSP homolog. The crystals provide means to obtain atomic modeling information of the specific amino acids and their atoms forming the binding site and that interact with molecules e.g., ligands or binding partners that bind to the KSP, via the binding site.

The crystals also provide modeling information regarding the protein-ligand interaction, as well as the structure of ligands bound thereto. The KSP crystal or a KSP homolog according to the present invention can be obtained by crystallizing it with a material or compound or molecule which binds to the herein disclosed binding site of the KSP. The KSP crystal according to the present invention includes KSP (human Eg5) and the material which binds to the specific binding site of KSP.

Preferred crystalline compositions of this invention are capable of diffracting X-rays to a resolution of better than about 3.5 Å, and more preferably to a resolution of about 2.6 Å or better, and even more preferably to a resolution of about 2.0 Å or better, and are useful for determining the three-dimensional structure of the material. (The smaller the number of angstroms, the better the resolution.)

The relative structural coordinates of the amino acid residues of the KSP motor domain, when the X-ray diffraction is obtained for the crystalline complex of KSP and a ligand compound described herein, are shown in Tables 1-4.

In another aspect, the present invention provides the three-dimensional structure of human KSP as well as the identification and characterization of a binding site there within. The identification of this site permits design and identification of compounds that bind to the ligand binding site and modulate KSP related activities. The compounds include inhibitors which specifically inhibit cell proliferation.

Of equal import is the fact that knowledge of the three-dimensional structure of the binding site of KSP provides a means for investigating the mechanism of action of the protein and tools for identifying inhibitors of its function.

As used herein, a ligand binding site also includes KSP or KSP analog residues which exhibit observable NMR perturbations in the presence of a binding ligand, such as any one of the herein disclosed inhibitors or any other ligand. While such residues exhibiting observable NMR perturbations may not necessarily be in direct contact with or immediately proximate to ligand binding residues, they may be critical to KSP residues for rational drug design protocols.

For example, knowledge of the three-dimensional structure of the ligand binding site allows one to design molecules, preferably pharmaceutical agents, capable of binding thereto, including molecules which are thereby capable of inhibiting the interaction of KSP with its native ligands, thereby inducing cell arrest.

Assays may be performed and the results analyzed to determine whether the agent is an inhibitor (i.e., the agent may reduce or prevent binding affinity between KSP and its native ligand/binding partner), or has no effect on the interaction between KSP and its native ligand. Agents identified using the foregoing methods, and preferably inhibitors of KSP, may then be tested as therapeutics in the treatment and/or prevention of hyper-proliferative cell disorders and other diseases that are also characterized by the presence of the hyper-proliferative cells such as cancer.

Once a KSP binding agent/inhibitor has been optimally selected or designed, as described above, substitutions may then be made in some of its atoms or side groups in order to improve or modify its selectivity and binding properties—that is its affinity for the ligand binding site disclosed herein. Generally, initial substitutions are conservative, i.e., the replacement group will have approximately the same size, shape, hydrophobicity and charge as the original group. Such substituted chemical compounds may then be analyzed for efficiency of fit the ligand binding site of KSP by the same computer methods described in detail above.

Various molecular analysis and rational drug design techniques are further disclosed in U.S. Pat. Nos. 5,834,228, 5,939,528 and 5,865,116, as well as in PCT Application No. PCT/US98/16879, published as WO 99/09148, the contents of which are hereby incorporated by reference.

In another aspect of the instant invention, the high quality single crystals of the KSP complexes comprising the KSP, ADP and the compounds described herein could be used to obtain single crystals of a KSP complex which comprises a compound that weakly binds to KSP or one or more weakly binding fragments of a compound that binds to KSP. This method may be termed intra-crystal ligand exchange. Thus, for example and not limiting in the scope of this embodiment, high quality single crystals of KSP-ADP-Compound 5-2b complex are exposed to the crystallization buffer described in the Materials and Methods which further contains 1 mM of a test compound that weakly binds to KSP. It is expected that the test compound will intercalate into the crystal and replace the compound 5-2b in the binding site. One or more molecular fragments of compounds that strongly bind to KSP may also be utilized in this technique.

X-ray diffraction data may be collected (as described in the Materials and Methods) from the high quality single crystals obtained by the intra-crystal ligand exchange technique. The 3-dimensional, tertiary structure of KSP bound to such a weakly binding compound could be utilized to guide the structural modification of the compound and, as a result, optimize the binding of the modified compound to KSP. The 3-dimensional tertiary structure of KSP bound to molecular fragment(s) could be utilized to guide in the identification of a new template for a compound having optimal binding to KSP.

Once the material is designed or selected, the affinity of the material to KSP may be calculated. For the inhibitor to be effective, it should have a high affinity for the ligand binding site, low energy difference between that energy calculated before and after binding. The affinity of the inhibitor may be measured by calculating the dissociation constant of the complex of KSP and the inhibitor. The dissociation constant is preferably 100 micromoles or less. The inhibitor preferably also maintains the bonding with KSP stably after binding. In order to do this, electrostatic repulsion such as charge-charge interactions, dipole-dipole and charge-dipole interactions between the inhibitor and KSP should not occur or be minimized. The sum of electrostatic interaction should be neutral or give a positive effect to the enthalpy of the bonding. Examples of programs designed for calculating such affinity include, but are not limited to as follows: Gaussian 92, revision C [M. J. Frisch, Gaussian, Inc., Pittsburgh, Pa. © 1992]; AMBER, version 4.0 [P. A. Kollman, University of California at San Fransisco, © 1994]; QUANTA/CHARMM [Molecular Simulations, Inc., Burlington, Mass. ©1994]; and Insight II/Discover (Biosysm Technologies Inc., San Diego, Calif., © 1994). Using the lead compound selected by the method, a stronger inhibitor can be made or designed. This process will be described below.

As well, any compound or anti-mitotic agent (lead compound) selected or designed in accordance with the methods disclosed herein can be changed or modified. Atoms, substituents or a part of the structure may be altered to increase the binding affinity to KSP. Generally, initial substitutions are conservative, i.e., the replacement group will have approximately the same size, shape, hydrophobicity and charge as the original group. It is noted that components known in the art to alter conformation should be avoided. The substituted chemical compounds may then be analyzed for fit with KSP by the same computer methods described herein.

After the material designed by the computer method described above is prepared and bound to KSP to produce a crystal, the 3-dimensional structure of the complex may be determined at high enough resolution (over 0.28 nm) using X-ray crystallographic methods. The information gained therefrom e.g., about the interaction between KSP and the inhibitor obtained from this can then be used to modify the inhibitor and to increase the affinity of the inhibitor for the ligand binding site of KSP.

Thus, for example, those atoms considered to be involved in binding to the ligand binding site of KSP disclosed herein can be mutated by exchanging one or more of the amino acid residues in the ligand binding site or in the motor domain of KSP that eventually effects the function of KSP on the underlying cell. As an example, if a cell's hyper-proliferative state is not effected by the mutated KSP, it may be surmised that the mutation very likely has not affected the function of KSP. In the alternative scenario, where the mutation decreases the hyper-proliferative state of the diseased cell, then one may surmise that the mutation has affected the ability of KSP to function in its intended purpose, e.g. hydrolyze ATP to ADP or bind microtubule etc. due to the substitution of the amino acid residue. This method can be used to identify amino acid residues in the original KSP which are important in the binding of the ligand to the binding site of KSP disclosed herein.

Once the amino acid residues in the ligand binding site of KSP have been identified as involved in the overall function attending KSP, the structure of the binding site can be identified based on the three-dimensional structure of KSP. Based on the structure of the binding site, a compound such as a peptide or other compound can be screened and designed which will fit into the three-dimensional model of the binding site.

Likewise, just as the three-dimensional modeling of KSP is provided by the present invention using the coordinates from the X-ray defraction patterns, these can be either analyzed directly to provide the three-dimensional structure (if of sufficiently high resolution). Alternatively, the atomic coordinates for the crystallized KSP, as provided herein, can be used for structure determination. The X-ray diffraction patterns obtained by methods of the present invention, can be provided on computer readable media, and used to provide electron density maps.

The electron density maps, provided by analysis of the X-ray coordinates of KSP complexed with Compound 5-2b, provided herein, may then be fitted using suitable computer algorithms to generate secondary, tertiary and/or quaternary structures and/or domains of KSP, which structures and/or domains are then used to provide an overall three-dimensional structure, as well as binding and/or active sites of KSP.

Knowledge obtained concerning KSP including the binding site defined herein can also be used to model the tertiary structure of related kinesin proteins, in particular members of the BimC protein family.

As an example, the structure of renin has been modeled using the tertiary structure of endothiapepsin as a starting point for the derivation. Model building of cercarial elastase and tophozoite cysteine protease were each built from known serine and cysteine proteases that have less than 35% sequence identity. The resultant models were used to design inhibitors in the low micromolar range. (Proc. Natl. Acad. Sci. 1993, 90, 3583). Furthermore, alternative methods of tertiary structure determination that do not rely on X-ray diffraction techniques and thus do not require crystallization of the protein, such as NMR techniques, are simplified if a model of the structure is available for refinement using the additional data gathered by the alternative technique. Thus, knowledge of the tertiary structure of the KSP binding site provides a significant window to the structure of the other kinesin family members. Thus, an embodiment of this invention envisions use of atomic coordinates of KSP protein, or fragment, analog or variant thereof, to model a KSP protein.

One skilled in the relevant art may use conventional molecular modeling methods to identify a ligand binding site of a KSP of another species. Specifically, coordinates provided by the present invention may be used to characterize a three-dimensional structure of the target KSP molecule, liganded or unliganded. Importantly, such a skilled artisan may, from such a structure, computationally visualize a putative binding site and identify and characterize other features based upon the coordinates provided herein. Such putative ligand binding sites may be further refined using chemical shift perturbations of spectra generated from various and distinct KSP complexes, e.g. from other species, competitive and non-competitive inhibition experiments, and/or by the generation and characterization of KSP or ligand mutants to identify critical residues or characteristics of the ligand binding site.

Such identification of a putative ligand binding site is of great import in rational drug design.

It is noted that in order to use the structural coordinates generated from the complex KSP described herein in Tables 1-4, it may be necessary to display the relevant coordinates as, or convert them to, a three-dimensional shape or graphical representation, or to otherwise manipulate them. In general, such a three-dimensional representation of the structural coordinates will find use in rational drug design, molecular replacement analysis, homology modeling, and mutation analysis. This is typically accomplished using any of a wide variety of commercially available software programs capable of generating three-dimensional graphical representations of molecules or portions thereof from a set of structural coordinates. The scientific art is replete with conventional software programs, which are incorporated by reference herein in their entirety. Refer to, for example, GRID (Oxford University, Oxford, UK); AUTODOCK (Scripps Research Institute, La Jolla, Calif.); Flo99 (Thistlesoft, Morris Township, N.J.) etc.

For storing, transferring and using such programs, a machine, such as a computer, is also contemplated, which produces a three-dimensional representation of the KSP binding site. The machine would comprise a machine-readable data storage medium comprising a data storage material encoded with machine-readable data. Machine-readable storage media comprising data storage material include conventional computer hard drives, floppy disks, DAT tape, CD-ROM, and other magnetic, magneto-optical, optical, floptical and other media which may be adapted for use with a computer. The machine further comprises a working memory for storing instructions for processing the machine-readable data, as well as a central processing unit (CPU) coupled to the working memory and to the machine-readable data storage medium for the purpose of processing the machine-readable data into the desired three-dimensional representation. As well, the machine of the present invention further comprises a display connected to the CPU so that the three-dimensional representation may be visualized by the user. Accordingly, when used with a machine programmed with instructions for using said data, e.g., a computer loaded with one or more programs of the sort identified above, the machine provided for herein is capable of displaying a graphical three-dimensional representation of the KSP complex described herein and set forth in Tables 1-4.

The structural coordinates of the present invention enable one to use various molecular design and analysis techniques in order to (i) solve the three-dimensional structures of related molecules, preferably molecular complexes such as those of other species or members of BimC family of proteins; as well as (ii) design, select, and synthesize chemical agents capable of favorably associating or interacting with a ligand binding site of a KSP molecule, wherein the molecular chemical entity would preferably inhibit KSP function including inducing mitotic arrest in cells contacted therewith.

Thus, the present invention provides a method for determining the molecular structure of a molecular complex whose structure is unknown, comprising the steps of obtaining the molecular complex whose structure is unknown, e.g., from a related species, and then generating NMR data there from. The NMR data from the molecular complex whose structure is unknown can then be compared to the structure data obtained from the KSP complex of the present invention. Then, 2D, 3D and 4D isotope filtering, editing and triple resonance NMR techniques can be used to conform the 3D structure described herein for the KSP complexes disclosed in Tables 1-4 to the NMR data from unknown target molecular complex. Alternatively, molecular replacement may be used to conform the 3D structure of the present invention to X-ray diffraction data from crystals of the unknown target molecular complex.

Molecular replacement involves correctly orienting and positioning the known structure into the crystal unit cell of the unknown structure. This is accomplished by a six dimensional (three positional and three rotational) search process that involves computation of a set of theoretical diffraction data using the known structure for every orientation and position searched and comparing it with the observed diffraction data of the unknown structure. The best match defines the correct position and orientation of the known structure in the unknown unit cell. This match offers phase information for use in conjunction with X-ray diffraction data of the unknown structure for the determination of its 3-dimensional structure.

In another aspect, this invention envisions use of atomic coordinates of the KSP protein disclosed herein, to design a chemical compound capable of associating with KSP or a fragment, analog or variant thereof.

For example, one method of this invention for evaluating the ability of a chemical entity to associate with any of the proteins or protein-ligand complexes set forth herein comprises the steps of: a) employing computational means to perform a fitting operation (docking) between the chemical entity and a binding pocket or other surface feature of the molecule or molecular complex; and b) analyzing the results of said fitting operation to quantify the association between the chemical entity and the binding pocket.

In another aspect, the invention envisions use of atomic coordinates of the KSP protein to design a model of ligands in the binding site defined herein.

Preferred embodiments of the aforementioned uses are those wherein the KSP protein comprises a binding site characterized by amino acid residues as set forth in FIG. 10.

As a general rule, one may use knowledge of the geography of the various regions of the ligand binding site disclosed herein, e.g. hydrophobic and/or hydrophilic to design KSP analogs (mutant) in which the overall KSP structure is not changed, but change does affect biological activity (“biological activity” being used here in its broadest sense to denote function). Thus, one may make changes to the amino acid sequences to effectively obtain a KSP analog/mutant that exhibits a greater affinity for its binding ligand. As well, one may correlate biological activity to structure. If the structure is not changed, and the mutation has no effect on biological activity, then the mutation has no biological function. If, however, the structure is not changed and the mutation does affect biological activity, then the residue (or atom) is essential to at least one biological function.

Similar molecular modeling is also provided by the present invention for rational drug design (RDD) of mimetics and ligands of KSP, “ligand” being used in the broadest sense, referring to any substance capable of observable binding to the KSP protein at the herein disclosed binding site. The drug design paradigm uses computer modeling programs to determine potential mimetics and ligands which are expected to interact with sites on the protein. The potential mimetics or ligands are then screened for activity and/or binding. For KSP-related mimetics or ligands, screening methods can be selected from assays for at least one biological activity of KSP, e.g., anti-mitotic activity. Thus, an embodiment of the invention envisions use of the structural information from the ligand/protein complexes found herein including the information derived therefrom in designing new chemical or biological moieties that bind tighter, bind more specifically, have better biological activity or have better safety profile than known ligands that bind KSP.

The computer modeling method disclosed herein can also be used to remodel the mimetics or ligands to improve the affinity or solubility, and produce an optimized pharmaceutical agent.

The resulting optimized mimetics or ligands can thereafter be prepared and the inhibitory activity for KSP can be tested in vitro and in vivo. If the test confirms that the material does indeed inhibit KSP, then the material or a derivative can be used as an anti-mitotic agent. Using the method as described above, the compound identified to have inhibitory activity may thereafter be used as a lead compound to obtain an improved inhibitor.

In order to confirm the affinity predicted by the computer modeling method, the dissociation constant of the complex may be experimentally measured.

The resulting mimetics or ligands are then provided by methods of the present invention and are useful for treating, inhibiting or preventing KSP-modulated diseases in animals, including humans. Preferably the ligands of the novel binding site provided herein are useful in the treatment or prevention of a hyper-proliferative disease, preferably cancer. Preferably, the ligand(s) identified by the methods described herein are useful in the treatment of cancer.

The ligands identified by the methods of this invention may be administered to mammals, preferably humans, either alone or, preferably, in combination with pharmaceutically acceptable carriers, excipients or diluents, in a pharmaceutical composition, according to standard pharmaceutical practice. The ligands can be administered orally or parenterally, including the intravenous, intramuscular, intraperitoneal, subcutaneous, rectal and topical routes of administration.

As used herein, the term “composition” is intended to encompass a product comprising the specified ingredients in the specific amounts, as well as any product which results, directly or indirectly, from combination of the specific ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. When a ligand according to this invention is administered into a human subject, the daily dosage will normally be determined by the prescribing physician with the dosage generally varying according to the age, weight, sex and response of the individual patient, as well as the severity of the patient's symptoms.

In one exemplary application, a suitable amount of a ligand of the novel KSP ligand binding site is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

Consequently, an object of the invention is to provide a method for determining the three-dimensional structure of a protein containing the ligand binding site as disclosed herein, or a complex of the protein with a ligand thereof, using homology modeling techniques and structural coordinates for a composition of this invention. Homology modeling involves constructing a model of an unknown structure using structural coordinates of one or more related proteins, protein domains and/or subdomains. Homology modeling may be conducted by fitting common or homologous portions of the protein or peptide whose three-dimensional structure is to be solved to the three-dimensional structure of homologous structural elements. Homology modeling can include rebuilding part or all of a three-dimensional structure with replacement of amino acids (or other components) by those of the related structure to be solved.

One of the objects of this invention is to provide three-dimensional structural information on new complexes of BimC family members of which KSP is a member with various ligands, as well as muteins or other variants of any of the foregoing. To that end, the invention provides for the use of the structural coordinates of a crystalline composition of this invention, or portions thereof, to solve, e.g., by molecular replacement, the three-dimensional structure of a crystalline form of such a ligand-protein complex, typically involving a protein containing at least one ligand binding site as disclosed herein. Doing so involves obtaining X-ray diffraction data for crystals of the protein-ligand complex for which one wishes to determine the three-dimensional structure. Then, one determines the three-dimensional structure of that protein or complex by analyzing the X-ray diffraction data using molecular replacement techniques with reference to the previous structural coordinates. As described in U.S. Pat. No. 5,353,236, for instance, molecular replacement uses a molecule having a known structure as a starting point to model the structure of an unknown crystalline sample.

Still further, the invention also includes compositions and methods for identifying binding sites of other members of the BimC protein family. The methods involve examining the surface of a protein of interest, preferably a kinesin, to identify residues that facilitate binding to the binding site. The residues can be identified by homology to the ligand binding site of human KSP described herein. Overlays and super-positioning with a three-dimensional model of a KSP binding site, or a portion thereof that contains a ligand binding site, also can be used for this purpose.

An alternative method of this invention provides for selecting from a database of chemical structures a compound capable of binding to a BimC family protein. The method starts with structural coordinates of a crystalline composition of the invention, e.g., coordinates defining the three-dimensional structure of a BimC family protein or a portion thereof e.g., the herein provided coordinates relative to human KSP. Points associated with that three-dimensional structure are characterized with respect to the extent of favorable interactions with one or more functional groups. A database of chemical structures is then searched for candidate compounds containing one or more functional groups disposed for favorable interaction with the protein based on the prior characterization. Compounds having structures which best fit the points of favorable interaction with the three-dimensional structure are thus identified.

An exemplary embodiment of the invention provides methods for identifying and designing small molecules that bind to the binding site using atomic models of KSP provided herein. The method involves modeling test compounds that fit spacially into the binding site of interest using an atomic structural model comprising a KSP binding site or portion thereof, screening the test compounds in a biological assay characterized by binding of a test compound to KSP, and identifying a test compound that binds to KSP.

Also provided is a method for identifying a potential inhibitor of KSP, comprising the steps of using a three-dimensional structure of a KSP binding site as defined by the relative structural coordinates set forth in Table 5 or the relative structural coordinates of the amino acids of FIG. 10 as set forth in Tables 14 to design or select a potential inhibitor, and obtaining or synthesizing said potential inhibitor. The inhibitor may be selected by screening an appropriate database, may be designed de novo by analyzing the steric configurations and charge potentials of an empty KSP binding site in conjunction with the appropriate software programs, or may be designed using characteristics of known inhibitors to create “hybrid” inhibitors. The inhibitor may then be contacted with KSP, and the effect of the inhibitor on KSP related function may be assessed. For instance, a potential inhibitor identified by this method may be contacted with KSP in the presence of one or two KSP substrates selected from ATP and microtubules, and determining the effect the potential inhibitor has on KSP ATPase activity. It is also within the confines of the present invention that a potential inhibitor may be designed or selected by identifying chemical entities or fragments capable of associating with KSP; and assembling the identified chemical entities or fragments into a single molecule to provide the structure of the potential inhibitor.

In furtherance of the above, there is provided a method for identifying an anti-mitotic agent comprising providing the atomic coordinates comprising the relative atomic structural coordinates of the amino acids of FIG. 10 as set forth in Tables 1-4 ±a root mean square deviation from the conserved backbone atoms of said amino acids of not more than about 2.00 Å thereof to a computerized modeling system; modeling compounds which fit spacially into the KSP binding site; and identifying in an assay for KSP activity a compound that inhibits or decreases the activity of the KSP through binding to the binding site.

Once the agent has been identified, it may be contacted with KSP and the effect the agent has on KSP may then be assessed. In addition, the agent may be contacted with KSP in the presence of a KSP binding molecule and the effect the agent has on binding between KSP and the KSP binding molecule may then be assessed.

Also disclosed herein is a process for identifying a potential anti-mitotic agent which upon binding to a human KSP inhibits cell proliferation, the process comprising the steps of:
a) exposing the KSP to a mixture of at least two potential ligands;
b) attempting to crystallize said KSP in the presence of said mixture;
c) if crystals are obtained, obtaining an X-ray diffraction pattern of the KSP crystal; and
d) determining whether a ligand/KSP complex is formed by comparing the electron density map calculated from the X-ray diffraction pattern of said KSP crystal when exposed to said mixture of said at least two potential ligands to the electron density map calculated from the X-ray diffraction pattern set forth in a table selected from Table 1, 2, 3 and 4.

Also provided herein is a method of identifying a compound that modulates the binding of a ligand to a ligand binding site of a human KSP, said method comprising: modeling test compounds that fit spatially into a KSP ligand binding site using an atomic structural model of a KSP binding site having the relative structural coordinates as set forth in a table selected from the group consisting of Tables 1, 2, 3 and 4 for the KSP amino acid residues 115 (M), 116(E), 117(G), 118(E), 119(R), 127(W), 130(D), 132(L), 133(A), 134(G), 136(I), 137(P), 160(L) 211(Y), 214(L), 215(E), 217(G), 218(A), 221(R) and 239(F), ±the root mean square deviation from the backbone atoms of said amino acids of not more than about 2.0 Å; screening the test compounds in an assay characterized by binding of a ligand to the ligand binding site; and identifying a test compound that modulates binding of said ligand to the KSP at its binding site.

Further provided is a method for identifying a potential inhibitor of human kinesin spindle protein (KSP), the method comprising the steps of:
(i) providing a three-dimensional structure of a ligand-bound KSP as defined by atomic coordinates set forth in a table selected from Tables 1, 2, 3 and 4;
(ii) comparing the three-dimensional coordinates of the ligand when it is bound to KSP as set forth in Table 1, 2, 3 or 4 to the three-dimensional coordinates of a compound in a database of compound structures; and
(iii) selecting from said database at least one compound that is structurally similar to said ligand when it is bound to said KSP, wherein the selected compound is a potential inhibitor of said KSP.

Also provided is a method for identifying an anti-mitotic agent which upon binding to a target human KSP inhibits cell proliferation, the method comprising the steps of:
a) exposing a target KSP to a mixture of at least two potential ligands;
b) attempting to crystallize said target KSP in the presence of said mixture;
c) obtaining a crystal of said target KSP exposed to said mixture to determine whether ligand/KSP complex is formed; and
d) identifying a potential anti-mitotic agent as one that binds to said KSP at a ligand binding site having the relative structural coordinates as set forth in Table 5 ±the root mean square deviation of not more than about 2.0 Å.

Further provided is a method for identifying an anti-mitotic agent which upon binding to a target human KSP inhibits cell proliferation, the method comprising the steps of:
(a) obtaining a crystal of KSP, where said KSP has been crystallized while exposed to a mixture of at least two potential ligands;
(b) determining whether a ligand/KSP complex is formed in said crystal; and
(c) identifying a potential anti-mitotic agent as one that binds to said KSP at a ligand binding site having the relative structural coordinates as set forth in Table 5 ±the root mean square deviation of not more than about 2.0 Å.

In the methods described hereinabove, potential ligands of KSP include the test compounds and Mg++ and ADP.

Also provided is a method of modulating, e.g., inhibiting the activity of a KSP. The method can be in vitro or in vivo. The method comprises administering, in vitro or in vivo, a sufficient amount of a compound that binds to the binding site disclosed herein.

Also provided is a method of identifying a compound that selectively inhibits the activity of one type of KSP compared to other KSPs or kinesins, e.g., a KSP of one species over another or a KSP over another member of the BimC family, of which KSP is a member. Thus, the method enables the identification of KSP and KSP like proteins in the same family, e.g., BimC or the KSP in one species over another. The method is exemplified by modeling test compounds that fit spacially and preferentially into a KSP ligand binding site of interest using an atomic structural model of a KSP ligand binding site, selecting a compound that interacts with one or more residues of the ligand binding site unique in the context of that site, and identifying in an assay for ligand binding activity a compound that selectively binds to the ligand binding site compared to other KSP. The unique features involved in receptor-selective ligand binding can be identified by comparing atomic models of different receptors or isoforms of the same type of receptor.

The present invention also provides for computer programs for the expression (such as visual display) of the KSP or analog three-dimensional structure, and further, a computer program which expresses the identity of each constituent of a KSP molecule and the precise location within the overall structure of that constituent, down to the atomic level.

There are many currently available computer programs for the expression of the three-dimensional structure of a molecule. Generally, these programs provide for inputting of the coordinates for the three-dimensional structure of a molecule (i.e., for example, a numerical assignment for each atom of a KSP molecule along an x, y, and z axis or the assignment for each atom of the binding site described in Tables 1-4), means to express (such as visually display) such coordinates, means to alter such coordinates and means to express an image of a molecule having such altered coordinates. One may program crystallographic information, i.e., the coordinates of the location of the atoms of a KSP binding site molecule in three dimension space, wherein such coordinates have been obtained from crystallographic analysis of said KSP molecule, into such programs to generate a computer program for the expression (such as visual display) of the KSP three-dimensional structure.

In furtherance of the above, the present invention provides a machine, such as a computer, programmed in memory with the coordinates of KSP or portions thereof, together with a program capable of converting the coordinates into a three-dimensional graphical representation of the structural coordinates on a display connected to the machine.

As well, there is provided a computer program for the expression of KSP's three-dimensional structure together with the structure of the novel KSP binding site. Preferred is the computer program QUANTA 2000, available from Molecular simulations or Insight II, version 4, available from Biosym, San Diego, Calif., with the coordinates of the amino acids of FIG. 10 as set forth in Tables 1-4 input. Preferred expression means are well known to a skilled artisan. Alternatively, the present KSP crystallographic coordinates and diffraction data are also deposited in the Protein Data Bank, Chemistry Department, Brookhaven National Laboratory, Upton, N.Y. 119723, USA. One may use these data in preparing a different computer program for expression of the three-dimensional structure of a KSP molecule or analog thereof.

Structural coordinates of a crystalline composition of this invention may be stored in a machine-readable form on a machine-readable storage medium, e.g. a computer hard drive, diskette, DAT tape, etc., for display as a three-dimensional shape or for other uses involving computer-assisted manipulation of, or computation based on, the structural coordinates or the three-dimensional structures they define. For example, data defining the three-dimensional structure of a KSP protein or portions or structurally similar homologues of such proteins, may be stored in a machine-readable storage medium, and may be displayed as a graphical three-dimensional representation of the protein structure, typically using a computer capable of reading the data from said storage medium and programmed with instructions for creating the representation from such data.

This invention thus encompasses a machine, such as a computer, having a memory which contains data representing the structural coordinates of a crystalline composition of this invention, e.g. the coordinates set forth in Tables 1-4, together with additional optional data and instructions for manipulating such data. Such data may be used for a variety of purposes, such as the elucidation of other related structures and drug discovery. For example, a machine having a memory containing such data aids in the rational design or selection of inhibitors of KSP binding or activity, including the evaluation of the ability of a particular chemical entity to favorably associate with KSP as disclosed herein, as well as in the modeling of compounds, proteins, complexes, etc. related by structural or sequence homology to KSP.

Thus, three-dimensional modeling of KSP provided by the present invention using the coordinates from the X-ray diffraction patterns can be entered into one or more computer programs for molecular modeling. Such molecular modeling programs generate atomic coordinates that reflect the secondary, tertiary and/or quaternary structures of the protein which contribute to its overall three-dimensional structure and provide information related to binding and/or active sites of the protein.

The present invention further contemplates the use of the structural coordinates of the present invention with standard homology modeling techniques to determine the unknown three-dimensional structure of a target molecule or molecular complex. Homology modeling involves constructing a model of an unknown structure using structural coordinates of one or more related protein molecules/molecular complexes or parts thereof (i.e., ligand binding sites). In general, homology modeling entails fitting common or homologous portions of the protein whose three-dimensional structure is to be solved to the three-dimensional structure of homologous structural elements in the known molecule, specifically using the relevant (i.e., homologous) structural coordinates provided in Tables 1-4. Homology may be determined using amino acid sequence identity, homologous secondary structure elements, and/or homologous tertiary folds. Homology modeling can include rebuilding part or all of a three-dimensional structure with replacement of amino acids (or other components) by those of the related structure to be solved. Examples of programs for homology modeling include, but are not limited to: QUANTA (Molecular Simulations, Inc.), Molecular Operating Environment or MOE (Chemical Computing Group, Inc. 2002), MODELLER (copyright © 1989-2002 Andrej Sali; Departments of Biopharmaceutical Sciences and Pharmaceutical Chemistry, and California Institute for Quantitative Biomedical Research, Mission Bay Genentech Hall, University of California San Francisco) and others.

In accordance with the above, a three-dimensional structure for the unknown molecule/molecular complex may be generated using the three-dimensional structure of the KSP molecule of the present invention, Tables 1-4, refined using a number of techniques well known in the art, and then used in the same fashion as the structural coordinates of the present invention, for instance, in applications involving molecular replacement analysis, homology modeling, and rational drug design.

Among other aspects, the coordinates in Table 1-4 define the relative relationship between the protein, the nucleotide and the ligand. Such sets of coordinates are dependent upon the particular coordinate system used. Those skilled in the art will recognize that rotation, translation or other mathematical manipulation of these coordinates may change the specific values of these coordinates, but the new set(s) will still define the relationship between the multiple components of the crystal structure disclosed herein.”

The determination of the three-dimensional structure of the ligand binding site of KSP as disclosed herein is advantageous over conventional drug assay techniques, in which the only way to identify such an agent is to screen thousands of test compounds until an agent having the desired inhibitory effect on a target compound is identified. Generally, such conventional screening methods are expensive, time consuming, and do not elucidate the method of action of the identified agent on the target compound. In sharp contrast, advancing X-ray, spectroscopic and computer modeling technologies allow researchers to visualize the three-dimensional structure of a targeted compound (i.e., KSP ligand binding site), and using such a three-dimensional structure to identify putative binding sites and then identify or design agents to interact with these binding sites. These agents can thereafter be screened for an inhibitory effect upon the target molecule. Consequently, an embodiment of the invention details a method for identifying a potential inhibitor of KSP. The proposed method comprises using a three-dimensional structure of KSP and the novel binding site of the invention as defined by the relative structural coordinates of Tables 1-4 and the relative structural coordinates of the amino acid residues of FIG. 10 as set forth in Table 1-4 to design or select a potential inhibitor of KSP activity, followed by synthesizing or obtaining the said potential inhibitor. The inhibitor may be selected by screening an appropriate database. Alternatively, it may be designed de novo by analyzing the steric configurations and charge potentials of a ligand bound KSP complex in conjunction with the appropriate software programs, or may be designed using characteristics of known inhibitors of KSP.

An entity/agent that interacts or associates with the ligand binding site of KSP may be identified by performing computer fitting analyses to identify an agent which interacts or associates with said site. Computer fitting analyses utilize various computer software programs that evaluate the “fit” between the binding site and the identified agent, by (a) generating a three-dimensional model of the ligand binding site using homology modeling or the atomic structural coordinates of the binding site in Tables 1-4, and (b) determining the degree of association between the binding site and the identified agent. The degree of association may be determined computationally by any number of commercially available software programs, or may be determined experimentally using standard binding assays.

Preferably, the method of the present invention includes the use of a ligand binding site characterized by the three-dimensional structure comprising the relative structural coordinates of amino acid residues listed in FIG. 10 as set forth in Tables 1-4 ±a root mean square deviation from the conserved backbone atoms of said amino acids of not more than about 2.0 Å, preferably not more than about 1.0 Å, and most preferably not more than about 0.5 Å. It is understood that the method of the present invention includes additional embodiments comprising conservative substitutions of the noted amino acids which result in the same structural coordinates of the corresponding residues in Tables 1-4 within the stated root mean square deviation.

The effect of an agent identified by computer fitting analyses on human KSP activity may be further evaluated computationally, or experimentally by competitive binding experiments or by contacting the identified agent with KSP and measuring the effect of the agent on the target's biological activity. Standard enzymatic assays may be performed and the results analyzed to determine whether the agent is an inhibitor of KSP activity (i.e., induce cell cycle arrest or inhibit the association of KSP with a microtubule as well as any other known activities attending a kinesin). Further tests may be performed to evaluate the selectivity of the identified agent to KSP with regard to other KSP proteins (other species) or other members of the BimC protein family.

Preferably, the agent designed or selected to interact with KSP is capable of associating with KSP and of assuming a three-dimensional configuration and orientation that complements the relevant ligand binding site of KSP.

Consequently, using these criteria, the structural coordinates of the KSP molecule as disclosed herein, and/or structural coordinates derived therefrom using molecular replacement or homology modeling, agents may be designed having increased potency and/or selectivity versus known inhibitors, e.g, by modifying the structure of known inhibitors or by designing new agents de novo via computational inspection of the three-dimensional configuration of KSP's novel ligand binding site described herein (relative structural coordinates of amino acid residues listed in FIG. 10 as set forth in Tables 1-4 and the relative structural coordinates set forth in Table 5).

As such, an embodiment of the invention proposes using the structural coordinates of Tables 1-4 of the present invention, or structural coordinates derived therefrom using molecular replacement or homology modeling techniques as discussed above to screen a database for agents that may act as potential inhibitors of KSP activity. As an example, the obtained structural coordinates of the present invention may be read into a software package and the three-dimensional structure analyzed graphically. A number of computational software packages may be used for the analysis of structural coordinates, e.g., Sybyl (Tripos Associates) etc. Additional software programs may be optionally used to check the coordinates with regard to features such as bond and atom types. If necessary, the three-dimensional structure may be modified and then energy minimized using the appropriate software until all of the structural parameters are at their equilibrium/optimal values. The energy minimized structure can then be superimposed against the original structure to make sure there are no significant deviations between the original and the energy minimized coordinates.

Once the specific interaction between KSP and a known inhibitor is determined, e.g., such as the information provided in Tables 1-4, docking studies with different inhibitors will allow one skilled in the art to generate initial models of new inhibitors bound to KSP. The integrity of these new models may be evaluated a number of ways, including constrained conformational analysis using molecular dynamics methods; that is where both KSP and the bound inhibitor are allowed to sample different three-dimensional conformational states until the most favorable state is reached or found to exist between the protein and the bound agent etc. Once models are obtained of the original known agent bound to KSP (Tables 1-4) and computer models of other molecules bound to KSP are as well obtained, strategies may be proposed determined for designing modifications into the inhibitors to improve their activity and/or enhance their selectivity.

For example, once a KSP binding agent has been optimally selected or designed, as described above, substitutions may then be made in some of its atoms or side groups in order to improve or modify its selectivity and binding properties for KSP. Generally, initial substitutions are conservative, i.e., the replacement group will have approximately the same size, shape, hydrophobicity and charge as the original group. Such substituted chemical compounds may then be analyzed for efficiency of fit to KSP by the same computer methods described in detail above. Further molecular analysis and rational drug design techniques are disclosed in U.S. Pat. Nos. 5,834,228, and 5,939,528 the contents of which are incorporated by reference in their entirety.

Thus, an exemplary embodiment of the invention envisions a method of three-dimensional modeling of a KSP protein, comprising the steps of:
(a) providing three-dimensional atomic coordinates derived from X-ray diffraction measurements of a KSP protein in a computer readable format;
(b) inputting the data from step (a) into a computer with appropriate software programs; and
(c) generating a three-dimensional structural representation of the KSP protein suitable for visualization and further computational manipulation.

This invention further provides for the use of the structural coordinates of a crystalline composition of this invention, or portions thereof, to identify reactive amino acids within the three-dimensional structure, preferably within or adjacent to a ligand binding site; to generate and visualize a molecular surface, such as a water-accessible surface or a surface comprising the space-filling van der Waals surface of all atoms; to calculate and visualize the size and shape of surface features of the protein or complex, e.g., ligand binding pockets; to locate potential H-bond donors and acceptors within the three-dimensional structure, preferably within or adjacent to a ligand binding site; to calculate regions of hydrophobicity and hydrophilicity within the three-dimensional structure, preferably within or adjacent to a ligand binding site; and to calculate and visualize regions on or adjacent to the protein surface of favorable interaction energies with respect to selected functional groups of interest (e.g. amino, hydroxyl, carboxyl, methylene, alkyl, alkenyl, aromatic carbon, aromatic rings, heteroaromatic rings, substituted and unsubstituted phosphates, substituted and unsubstituted phosphonates, substituted and unsubstituted fluoro and difluorophosphonates; etc.). One may use the foregoing approaches for characterizing the protein and its interactions with moieties of potential ligands to design or select compounds capable of specific covalent attachment to reactive amino acids (e.g., cysteine) and to design or select compounds of complementary characteristics (e.g., size, shape, charge, hydrophobicity/hydrophilicity, ability to participate in hydrogen bonding, etc.) to surface features of the protein, a set of which may be preselected. Using the structural coordinates, one may also predict or calculate the orientation, binding constant or relative affinity of a given ligand to the protein in the complexed state, and use that information to design or select compounds of improved affinity.

In such cases, the structural coordinates of the KSP protein, or portion or complex thereof, are entered in machine readable form into a machine programmed with instructions for carrying out the desired operation and containing any necessary additional data, e.g. data defining structural and/or functional characteristics of a potential ligand or moiety thereof, defining molecular characteristics of the various amino acids, etc.

The present invention is additionally directed to a method of determining the three-dimensional structure of a molecule or molecular complex whose structure is unknown, comprising the steps of first obtaining crystals of the molecule or molecular complex whose structure is unknown, and then generating X-ray diffraction data from the crystallized molecule or molecular complex and/or generating NMR data from the solution of the molecule or molecular complex. The generated diffraction or spectroscopy data from the molecule or molecular complex can then be compared with the solution coordinates or three-dimensional structure of KSP as disclosed herein, and the three-dimensional structure of the unknown molecule or molecular complex conformed to the KSP structure using standard techniques such as molecular replacement analysis, 2D, 3D and 4D isotope filtering, editing and triple resonance NMR techniques, and computer homology modeling. Alternatively, a three-dimensional model of the unknown molecule may be generated by generating a sequence alignment between KSP and the unknown molecule, based on any or all of amino acid sequence identity, secondary structure elements or tertiary folds, and then generating by computer modeling a three-dimensional structure for the molecule using the three-dimensional structure of, and sequence alignment with, KSP.

Preferred embodiments of the aforementioned methods are those methods wherein the KSP protein comprises a binding site characterized by amino acid residues described in FIG. 10.

This invention also provides peptidomimetic methods for designing a compound capable of binding to a KSP protein or KSP homolog. One such method involves graphically displaying a three-dimensional representation based on coordinates defining the three-dimensional structure of a KSP family protein or a portion thereof complexed with a ligand. Interactions between portions of a ligand and the protein may then be analyzed in order to identify candidate moieties for replacement. One or more portions of the ligand which interact with the protein may be replaced with substitute moieties selected from a knowledge base of one or more candidate substitute moieties, and/or moieties may be added to the ligand to permit additional interactions with the protein.

In another aspect of the instant invention, the structural coordinates of a crystalline composition of this invention, or portions thereof, may be used to identify one or more pharmacophores of a chemical compound that binds to the ligand binding site. Such a pharmacophore is described as a set of atoms, chemical groups, pseudo-atoms or vectors, and the relative positions in space of each of these pharmacophore features. Each feature, alone or in combination with its relative position, forms a pharmacophore parameter. Thus, the pharmacophore includes the pharmacophore features, and the relative position of each descriptor with regard to all other descriptors comprising the pharmacophore.

Pharmacophore models can be constructed either directly or indirectly. In the direct method, the pharmacophore feature spatial centers are inferred from studying the X-ray structural coordinates or NMR structure of a receptor-ligand complex, followed by a shape-complementarity function analysis of the receptor binding site, usually performed using a computer and a computer-readable medium. In the indirect method, the structure of the receptor is unknown and the pharmacophore feature spatial centers are inferred by overlaying the three-dimensional conformations of active compounds and finding the common, overlapping functional groups.

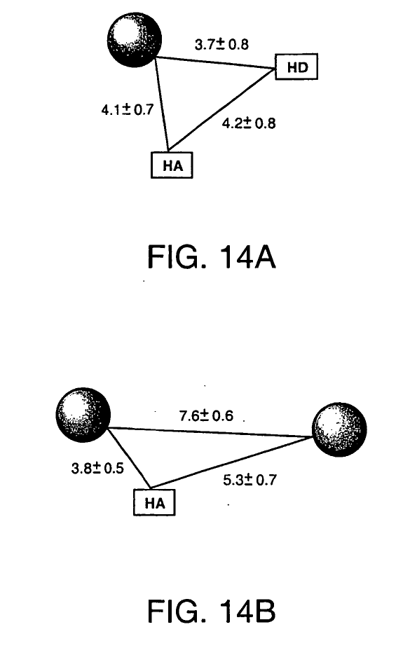

The pharmacophore models of the present invention, obtained by combining both direct and indirect methods, are herein described, by way of example only and without any intention of being limiting, with reference to FIGS. 14A and B.

The first model pharmacophore (FIG. 14A) is represented by three pharmacophore features having the planar orientation shown: a sphere indicating the center of an aryl, heteroaryl or cycloalkyl ring (or, in general, of a hydrophobic group), and two small boxes (labeled HA and HD), representing the heterocenters of a hydrogen bond acceptor and a hydrogen bond donor, respectively. The second model pharmacophore (FIG. 14B) is represented by three pharmacophore features: two spheres indicating the centers of two aryl, heteroaryl or cycloalkyl rings (or hydrophobic groups in general), and a small box representing the heteroatomic center of a hydrogen bond acceptor (HA).

As used herein, “aryl” is intended to mean any stable monocyclic or bicyclic carbon ring of up to 7 atoms in each ring, wherein at least one ring is aromatic. Examples of such aryl elements include phenyl, naphthyl, tetrahydronaphthyl, indanyl and biphenyl. In cases where the aryl substituent is bicyclic and one ring is non-aromatic, it is understood that attachment is via the aromatic ring.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. In an embodiment of the instant invention, heteroaryl does not include quinazolinone.

As used herein, “cycloalkyl” is intended to include monocyclic saturated aliphatic hydrocarbon groups having the specified number of carbon atoms. For example, “cycloalkyl” includes cyclopropyl, methyl-cyclopropyl, 2,2-dimethyl-cyclobutyl, 2-ethyl-cyclopentyl, cyclohexyl, and so on. In an embodiment of the invention the term “cycloalkyl” includes the groups described immediately above and further includes monocyclic unsaturated aliphatic hydrocarbon groups. For example, “cycloalkyl” as defined in this embodiment includes cyclopropyl, methyl-cyclopropyl, 2,2-dimethyl-cyclobutyl, 2-ethyl-cyclopentyl, cyclohexyl, cyclopentenyl, cyclobutenyl and so on.

The, cycloalkyl, aryl, heteroaryl and heteroaryl substituents may be substituted or unsubstituted, unless specifically defined otherwise. For example, an aryl may be substituted with one, two or three substituents selected from OH, alkyl, halogen, alkoxy or dialkylamino.

The active structural motifs designated herein as the model pharmacophores of the present invention can be used to screen libraries of molecules for the existence of a predefined structural motif, and in particular identifying molecules that meet the constraints imposed by the pharmacophore. The pharmacophore feature spatial centers are globally associated with a specific biological activity. The molecules being evaluated may be designed de novo using computer methods, or alternatively, be either a scaffold or a full chemical entity (e.g., chosen from a library of compounds). Using the model pharmacophores disclosed herein one of ordinary skill may predict the inhibitory potency of a compound based upon its fit with any of these two pharmacophore models shown in FIG. 14A and B.

In an embodiment, the compound identified by the use of a pharmacophore model described herein has a binding affinity for KSP of about 0.1 nM to about 100 nM. In a further embodiment, the binding affinity range is from about 1 nM to about 20 nM.

In an embodiment, the compound identified by its fit with the pharmacophore model of FIG. 14A does not incorporate a 2-thioxo-1,2,3,4-tetrahydropyrimidine moiety, a dihydropyrimidine moiety or a 5,6,11,11a-tetrahydro-1H-imidazo[1′,5′:1,6]-pyrido[3.4-b]indole-1,3(2H)-dione moiety.

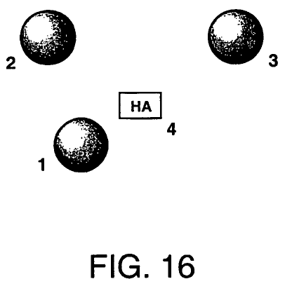

An additional pharmacophore model is illustrated by FIG. 16. The pharmacophore model of FIG. 16 is represented by four pharmacophore features: three spheres indicating the centers of aryl, heteroaryl or cycloalkyl rings (or hydrophobic groups in general), and a small box representing the heteroatomic center of a hydrogen bond acceptor (HA). In reference to FIG. 16, the distances in Å between the pharmacophore features are listed in the following table:

| | 1 | 2 | 3 | 4 |
|---|---|---|---|---|
| 1 | — | | | |
| 2 | 5.1 ± 0.6 | — | | |
| 3 | 8.5 ± 0.7 | 6.9 ± 0.7 | — | |
| 4 | 3.7 ± 0.5 | 5.8 ± 0.6 | 5.7 ± 0.7 | — |

In an embodiment, the compound identified by its fit with the pharmacophore model of FIG. 16 does not incorporate a quinazolinone, phenothiazine, thienopyrimidinone, furanopyrimidinone, azolopyrimidinone, thiazolopyrimidine, cycloalkylpyrimidinone or triphenylmethane moiety. In a further embodiment, the compound identified by its fit with the pharmacophore model of FIG. 16 does not incorporate a quinazolinone, phenothiazine or triphenylmethane moiety.

In an embodiment, the compound identified by its fit with the pharmacophore model of FIG. 14B does not incorporate a quinazolinone, phenothiazine, thienopyrimidinone, furanopyrimidinone, azolopyrimidinone, thiazolo-pyrimidine, cycloalkylpyrimidinone or triphenylmethane moiety. In a further embodiment, the compound identified by its fit with the pharmacophore model of FIG. 14B does not incorporate a quinazolinone, phenothiazine or triphenylmethane moiety.

The degree of fit of a particular compound structure to the pharmacophore models is calculated by determining, using computer methods, if the compound possesses the chemical features of the pharmacophore model and if the features can adopt the necessary three-dimensional arrangement to fit the model. The modeling program will indicate those features in the pharmacophore model having a fit with the particular compound or chemical feature of the compound being tested. The term “fit” when referring to a compound and a pharmacophore or binding site includes both compounds that occupy only the spatial area of the pharmacophore or binding site and compounds of which the chemical features or a portion of the molecule occupy the spatial area of the pharmacophore or binding site.

Fitting of a compound to the ligand binding site volume can be done in a number of different ways using computational methods well known by those skilled in the art. Visual inspection and manual docking of compounds into the induced-fit active site volume can be done using molecular modeling software such as QUANTA (Molecular Simulations, Burlington, Mass., 1992), SYBYL (Tripos Associates, Inc., St. Louis, Mo., 1992), AMBER (Weiner et al., J. Am. Chem. Soc., 106: 765-784, 1984), CHARMM (Brooks et al., J. Comp. Chem., 4: 187-217, 1983) or other modeling programs known to those of skill in the art. This modeling step may be followed by energy minimization using standard force fields, such as CHARMM and AMBER, or others. More specialized modeling programs include MCSS (Miranker & Karplus, Function and Genetics, 11: 29-34, 1991), GRID (Goodford et al., J. Med. Chem., 28: 849-857, 1985), AUTODOCK (Goodsell & Olsen, Proteins: Structure, Function and Genetics, 8: 195-202, 1990), and DOCK (Kuntz et al., J. Mol. Biol., 161: 269-288, 1982). In addition, inhibitor compounds may be constructed de novo in the empty active site or in the active site including some portions of a known inhibitor using computer programs such as LEGEND (Nishibata & Itai, Tetrahedron, 47: 8985, 1991), LeapFrog (Tripos Associates, St. Louis, Mo.), LUDI (Bohm, J. Comp. Aid. Molec. Design, 6: 61-78, 1992), AutoLudi (Accelrys Inc., San Diego, Calif.) or others.

Another aspect of the invention relates to a complementary protein having a structure substantially complementary to the three-dimensional structure according to Tables 1-4; or to a medicinally effective part thereof, particularly a ligand binding region. A complementary protein is one whose three-dimensional structure is substantially complementary to the Tables 1-4 structure or a part thereof, such that the complementary structure may bind thereto and may form a complex. The lifetime of the complex may be long in the case of an inhibiting complementary protein. Of course, binding will also require an appropriate choice of amino acid sequence. Such a complementary protein may act as an inhibitor of KSP. Such inhibitors may be used in vivo or in vitro to modify the activity of KSP.

In the pharmaceutical industry, new or known compounds are routinely screened for new uses employing a variety of known in vitro or in vivo screens. Often such screens involve complex natural substances and are correspondingly expensive to carry out, and the result may be difficult to interpret. The knowledge of the three-dimensional protein structure according to the invention allows a preliminary screening to be carried out on the basis of the three-dimensional structure of a region thereof, and the structural similarity of a molecule which is being screened. This is usually carried out in conjunction with a knowledge of the amino sequence of the region. Such screening can conveniently be carried out using computer modeling techniques, which match the three-dimensional structure of the protein or part thereof (or complementary protein or part thereof) with the structure of the molecule being screened, thereby allowing one to predict potential inhibitor activity.

The binding of a ligand to the novel binding site of the instant invention and the formation of the novel binding pocket as a result can als6 be indirectly assessed by spectroscopically determining the shift in the fluorescence of the amino acid 127 tryptophan residue. Thus it has been discovered that the fluorescent emission of Trp127 is modulated when KSP is treated with one of the inhibitors described above in the presence of a nucleotide or nucleotides.

A further embodiment of the instant invention is an in vitro assay for the determination of binding of a test compound to the novel KSP binding site described herein. The assay comprises the steps of:
1. contacting KSP with the test compound and a nucleotide and measuring the fluorescence of the mixture at the peak emission wavelength for Trp127 in KSP;
2. contacting KSP with a nucleotide and measuring the fluorescence of the mixture at the peak emission wavelength for Trp127 in KSP; and
3. comparing the fluorescence of the mixture of KSP, the test compound and the nucleotide with the fluorescence of the mixture of KSP with the nucleotide alone.

In another embodiment of the in vitro fluorescence assay the nucleotide is selected from ADP and AMPPNP (a non-hydrolysable analog of ATP, adenosine 5′-(β,γ-imido)triphosphate tetralithium salt hydrate).

In an embodiment of the in vitro fluorescence assay the mixtures additionally contain a source of magnesium ion. Preferably the source of magnesium ion is MgCl₂.

In another embodiment of the in vitro fluorescence assay the measurement of the fluorescence of the KSP, test compound and nucleotide mixture is performed at several different concentrations of the test compound.

Because the KSP kinesin's three-dimensional structure is uniquely suited to the formation of the novel binding pocket of the instant invention, the methods of identification of compounds that bind to the novel binding pocket described herein, such as the fluorescence assay described above, may be used to identify selective inhibitors of KSP which may not inhibit other mitotic kinesins. Such identification of a selective KSP inhibitor may offer particular advantages over an inhibitor which is competitive with the binding of the nucleotide substrate of KSP or which binds to the site of microtubule binding.

A still further aspect of the invention relates to antibodies (including monoclonal antibodies) directed to the KSP protein or complementary protein, for the detection thereof or for the modulation of its medicinal activity, it being understood that the antibody is specific for the KSP-ligand, e.g., inhibitor bound conformation.

Compounds of the structures selected or designed by any of the foregoing means may be tested for their ability to bind to a KSP protein, inhibit the binding of a KSP protein to a natural or non-natural ligand therefor, and/or inhibit a biological function mediated by a KSP protein or a BimC family member.

Finally, the present invention provides agents or inhibitors designed or selected using the methods disclosed herein. Such compounds may be utilized as described in the following sections.

Utilities

The compounds designed or selected using the methods of the invention find use in a variety of applications. As will be appreciated by those in the art, mitosis may be altered in a variety of ways; that is, one can affect mitosis either by increasing or decreasing the activity of a component in the mitotic pathway. Stated differently, mitosis may be affected (e.g., disrupted) by disturbing equilibrium, either by inhibiting or activating certain components. Similar approaches may be used to alter meiosis.

In a preferred embodiment, the compounds designed or selected using the methods of the invention are used to modulate mitotic spindle formation, thus causing prolonged cell cycle arrest in mitosis. By “modulate” herein is meant altering mitotic spindle formation, including increasing and decreasing spindle formation. By “mitotic spindle formation” herein is meant organization of microtubules into bipolar structures by mitotic kinesins. By “mitotic spindle dysfunction” herein is meant mitotic arrest and monopolar spindle formation.

The compounds designed or selected using the methods of the invention are useful to bind to and/or modulate the activity of a mitotic kinesin. In a preferred embodiment, the mitotic kinesin is a member of the bimC subfamily of mitotic kinesins (as described in U.S. Pat. No. 6,284,480, column 5). In a further preferred embodiment, the mitotic kinesin is human KSP, although the activity of mitotic kinesins from other organisms may also be modulated by the compounds of the present invention. In this context, modulate means either increasing or decreasing spindle pole separation, causing malformation, i.e., splaying, of mitotic spindle poles, or otherwise causing morphological perturbation of the mitotic spindle. Also included within the definition of KSP for these purposes are variants and/or fragments of KSP. See PCT Publ. WO 01/31335: “Methods of Screening for Modulators of Cell Proliferation and Methods of Diagnosing Cell Proliferation States”, filed Oct. 27, 1999, hereby incorporated by reference in its entirety. In addition, other mitotic kinesins may be inhibited by the compounds of the present invention.

The compounds designed or selected using the methods of the invention are used to treat cellular proliferation diseases. Disease states which can be treated by the methods and compositions provided herein include, but are not limited to, cancer (further discussed below), autoimmune disease, arthritis, graft rejection, inflammatory bowel disease, proliferation induced after medical procedures, including, but not limited to, surgery, angioplasty, and the like. It is appreciated that in some cases the cells may not be in a hyper- or hypoproliferation state (abnormal state) and still require treatment. For example, during wound healing, the cells may be proliferating “normally”, but proliferation enhancement may be desired. Similarly, as discussed above, in the agriculture arena, cells may be in a “normal” state, but proliferation modulation may be desired to enhance a crop by directly enhancing growth of a crop, or by inhibiting the growth of a plant or organism which adversely affects the crop. Thus, in one embodiment, the invention herein includes application to cells or individuals afflicted or impending affliction with any one of these disorders or states.

The compounds, compositions and methods provided herein are particularly deemed useful for the treatment of cancer including solid tumors such as skin, breast, brain, cervical carcinomas, testicular carcinomas, etc. More particularly, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple mycloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematolopic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

The compounds designed or selected using the methods of the instant invention may also be useful as antifungal agents, by modulating the activity of the fungal members of the bimC kinesin subgroup, as is described in U.S. Pat. No. 6,284,480.

The compounds designed or selected using the methods of this invention may be administered to mammals, preferably humans, either alone or, preferably, in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

As used herein, the term “composition” is intended to encompass a product comprising the specified ingredients in the specific amounts, as well as any product which results, directly or indirectly, from combination of the specific ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds designed or selected using the methods disclosed herein may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound are employed. (For purposes of this application, topical application shall include mouth washes and gargles.)

The compounds designed or selected using the methods of the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a compound according to this invention is administered into a human subject, the daily dosage will normally be determined by the prescribing physician with the dosage generally varying according to the age, weight, sex and response of the individual patient, as well as the severity of the patient's symptoms.

In one exemplary application, a suitable amount of compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

The compounds designed or selected using the methods disclosed herein (hereafter referred to as the “instant compounds”) are also useful in combination with known therapeutic agents and anti-cancer agents. For example, instant compounds are useful in combination with known anti-cancer agents. Combinations of the presently disclosed compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, and agents that interfere with cell cycle checkpoints. The instant compounds are particularly useful when co-administered with radiation therapy.

In an embodiment, the instant compounds are also useful in combination with known anti-cancer agents including the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N4-carboxyphenyl retinamide.

“Cytotoxic/cytostatic agents” refer to compounds which cause cell death or inhibit cell proliferation primarily by interfering directly with the cell's functioning or inhibit or interfere with cell myosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteosome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, and 4-demethoxy-3-deamino-3-aziridinyl4-methylsulphonyl-daunorubicin (see WO 00/50032).

An example of a hypoxia activatable compound is tirapazamine.

Examples of proteosome inhibitors include but are not limited to lactacystin and MLN-341 (Velcade).

Examples of microtubule inhibitors/microtubule-stabilising agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro4-methoxyphenyl) benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797. In an embodiment the epothilones are not included in the microtubule inhibitors/microtubule-stabilising agents.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydroOxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in PCT Publications WO 01/30768 and WO 01/98278, and pending U.S. Ser. Nos. 60/338,779 (filed Dec. 6, 2001), 60/338,344 (filed Dec. 6, 2001), 60/338,383 (filed Dec. 6, 2001), 60/338,380 (filed Dec. 6, 2001), 60/338,379 (filed Dec. 6, 2001) and 60/344,453 (filed Nov. 7, 2001). In an embodiment inhibitors of mitotic kinesins include, but are not limited to inhibitors of KSP, inhibitors of MKLP1, inhibitors of CENP-E, inhibitors of MCAK and inhibitors of Rab6-KIFL. “Inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, 3-aminopyridine-2-carboxaldehyde thiosemicarbazone and trastuzumab.

Examples of monoclonal antibody targeted therapeutic agents include those therapeutic agents which have cytotoxic agents or radioisotopes attached to a cancer cell specific or target cell specific monoclonal antibody. Examples include Bexxar. “HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein.

Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

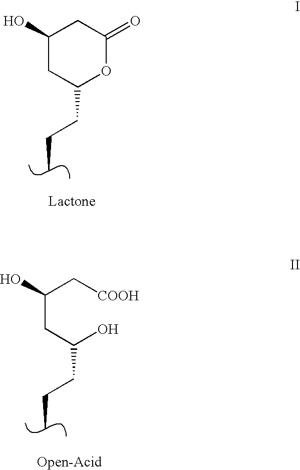

In HMG-CoA reductase inhibitors where an open-acid form can exist, salt and ester forms may be formed from the open-acid, and all such forms are included within the meaning of the term “HMG-CoA reductase inhibitor” as used herein. In an embodiment, the HMG-CoA reductase inhibitor is selected from lovastatin and simvastatin, and in a further embodiment, simvastatin. Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenz-imidazole, diethylamine, piperazine, and tris(hydroxymethyl) aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). Examples of prenyl-protein transferase inhibiting compounds include (±)-6-[amino(4-chlorophenyl)(1-methyl-1H-imidazol-5-yl)methyl]-4-(3-chlorophenyl)-1-methyl-2(1H)-quinolinone, (−)-6-[amino(4-chlorophenyl)(1-methyl-1H-imidazol-5-yl)methyl]-4-(3chlorophenyl)-1-methyl-2(1H)-quinolinone, (+)-6-[amino(4-chlorophenyl)(1-methyl-1H-imidazol-5-yl)methyl]-4-(3-chlorophenyl)-1-methyl-2(1H)-quinolinone, 5(S)-n-butyl-1-(2,3-dimethylphenyl)-4-[1-(4-cyanobenzyl)-5-imidazolylmethyl]-2-piperazinone, (S)-1-(3-chlorophenyl)4-[1-(4-cyanobenzyl)-5-imidazolylmethyl]-5-[2-(ethanesulfonyl)methyl)-2-piperazinone, 5(S)-n-Butyl-1-(2-methylphenyl)-4-[1-(4-cyanobenzyl)-5-imidazolylmethyl]-2-piperazinone, 1-(3-chlorophenyl) -4-[1-(4-cyanobenzyl)-2-methyl-5-imidazolylmethyl]-2-piperazinone, 1-(2,2-diphenylethyl)-3-[N-(1-(4-cyanobenzyl)-1H-imidazol-5-ylethyl)carbamoyl]piperidine, 4-{5-[4-hydroxymethyl-4-(4-chloropyridin-2-ylmethyl)-piperidine-1-ylmethyl]-2-methylimidazol-1-ylmethyl}benzonitrile, 4-{5-[4-hydroxymethyl-4-(3-chlorobenzyl)-piperidine-1-ylmethyl]-2-methylimidazol-1-ylmethyl}benzonitrile, 4-{3-[4-(2-oxo-2H-pyridin-1 -yl)benzyl]-3H-imidazol-4-ylmethyl}benzonitrile, 4-{3-[4-(5-chloro-2-oxo-2H-[1,2′]bipyridin-5′-ylmethyl]-3H-imidazol4-ylmethyl}benzonitrile, 4-{3-[4-(2-oxo-2H-[1,2′]bipyridin-5′-ylmethyl]-3H-imidazol4-ylmethyl}benzonitrile, 4-[3-(2-oxo-1-phenyl-1,2-dihydropyridin-4-ylmethyl)-3H-imidazol-4-ylmethyl}benzonitrile, 18,19-dihydro-19-oxo-5H,17H-6,10:12,16-dimetheno-1H-imidazo[4,3-c][1,11,4]dioxaazacyclo-nonadecine-9-carbonitrile, (±)-19,20-dihydro-19-oxo-5H-18,21-ethano-12,14-etheno-6,10-metheno-22H-benzo[d]imidazo[4,3-k][1,6,9,12]oxatriaza-cyclooctadecine-9-carbonitrile, 19,20-dihydro-19-oxo-5H,17H-18,21-ethano-6,10:12,16-dimetheno-22H-imidazo[3,4-h][1,8,11,14]oxatriazacycloeicosine-9-carbonitrile, and (±)-19,20-dihydro-3-methyl-19-oxo-5H-18,21-ethano-12,14-etheno-6,10-metheno-22H-benzo [d]imidazo[4,3-k][1,6,9,12]oxa-triazacyclooctadecine-9-carbonitrile.

Other examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359.

For an example of the role of a prenyl-protein transferase inhibitor on angiogenesis see European J. of Cancer, Vol. 35, No. 9, pp. 1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins, GPIIb/IIIa antagonists (such as tirofiban), warfarin, thrombin inhibitors and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in U.S. Ser. Nos. 60/310,927 (filed Aug. 8, 2001) and 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Inhibitors of cell proliferation and survival signalling pathway” refer to compounds that inhibit signal transduction cascades downstream of cell surface receptors. Such agents include inhibitors of serine/threonine kinases (including but not limited to inhibitors of Akt such as described in WO 02/083064, WO 02/083139, WO 02/083140 and WO 02/083138), inhibitors of Raf kinase (for example BAY43-9006), inhibitors of MEK (for example CI-1040 and PD-098059), inhibitors of mTOR (for example Wyeth CCI-779), and inhibitors of PI3K (for example LY294002).

The combinations with NSAID's are directed to the use of NSAID's which are potent COX-2 inhibiting agents. For purposes of this specification an NSAID is potent if it possess an IC₅₀ for the inhibition of COX-2 of 1 μM or less as measured by cell or microsomal assays.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. 5,698,584, issued Dec. 16, 1997, U.S. Pat. No. 5,710,140, issued Jan. 20,1998, WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999, all of which are hereby incorporated by reference.

Inhibitors of COX-2 that are particularly useful in the instant method of treatment are:
3-phenyl-4-(4-(methylsulfonyl)phenyl)-2-(5H)-furanone; and 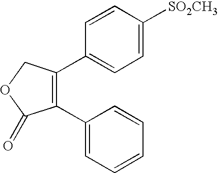
5-chloro-3-(4-methylsulfonyl)phenyl-2-(2-methyl-5-pyridinyl)pyridine, 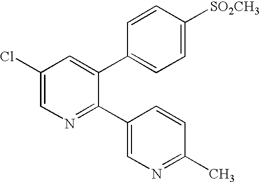
   or a pharmaceutically acceptable salt thereof

General and specific synthetic procedures for the preparation of the COX-2 inhibitor compounds described above are found in U.S. Pat. No. 5,474,995, issued Dec. 12, 1995, U.S. Pat. No. 5,861,419, issued Jan. 19, 1999, and U.S. Pat. No. 6,001,843, issued Dec. 14, 1999, all of which are herein incorporated by reference.

Compounds that have been described as specific inhibitors of COX-2 and are therefore useful in the present invention include, but are not limited to, the following: 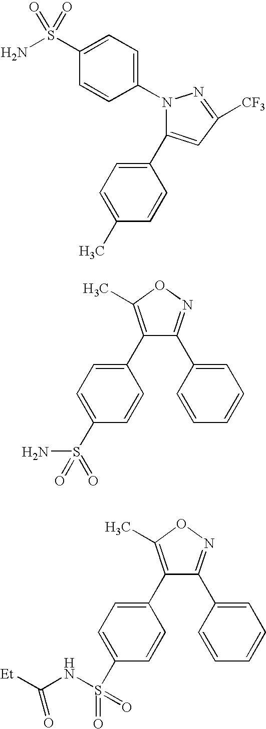
or a pharmaceutically acceptable salt thereof.

Compounds which are described as specific inhibitors of COX-2 and are therefore useful in the present invention, and methods of synthesis thereof, can be found in the following patents, pending applications and publications, which are herein incorporated by reference: WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999.

Compounds which are specific inhibitors of COX-2 and are therefore useful in the present invention, and methods of synthesis thereof, can be found in the following patents, pending applications and publications, which are herein incorporated by reference: U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued July 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, and U.S. Pat. No. 5,710,140, issued Jan. 20,1998.

Other examples of angiogenesis inhibitors include, but are not limited to, endostatin, ukrain, ranpirnase, IM862, 5-methoxy4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the (αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9, 12-epoxy-1H-diindolo[1,2,3-fg:3′,2′, 1′-k1]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N4-chlorophenyl-4-(4-pyridylmethyl)-1-phthalazinamine, and EMD121974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999;274:9116-9121; Invest. Ophthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI1262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy) phenoxy)propoxy)-2-ethylchromane-2-carboxylic acid (disclosed in U.S. Ser. Nos. 60/235,708 and 60/244,697).

Another embodiment of the instant invention is the use of the presently disclosed compounds in combination with gene therapy for the treatment of cancer. For an overview of genetic strategies to treating cancer see Hall et al (Am J Hum Genet 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, Aug. 1998;5(8):1105-13), and interferon gamma (J Immunol 2000;164:217-222).

The compounds designed or selected using the methods of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

A compound designed or selected using the methods of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. For the treatment or prevention of emesis that may result upon administration of the instant compounds, conjunctive therapy with an anti-emesis agent selected from a neurokinin-1 receptor antagonist, a 5HT3 receptor antagonist and a corticosteroid is preferred.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)4-(3-(5-oxo-1H,4H-1,2,4-triazolo)methyl)morpholine, or a pharmaceutically acceptable salt thereof, which is described in U.S. Pat. No. 5,719,147.

A compound designed or selected using the methods of the instant invention may also be administered with an agent useful in the treatment of anemia. Such an anemia treatment agent is, for example, a continuous eythropoiesis receptor activator (such as epoetin alfa).

A compound designed or selected using the methods of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound designed or selected using the methods of the instant invention may also be administered with an immunologic-enhancing drug, such as levamisole, isoprinosine and Zadaxin.

Thus, the scope of the instant invention encompasses the use of the compounds designed or selected using the methods disclosed herein in combination with a second compound selected from:

1) an estrogen receptor modulator,

2) an androgen receptor modulator,

3) retinoid receptor modulator,

4) a cytotoxic/cytostatic agent,

5) an antiproliferative agent,

6) a prenyl-protein transferase inhibitor,

7) an HMG-CoA reductase inhibitor,

8) an HIV protease inhibitor,

9) a reverse transcriptase inhibitor,

10) an angiogenesis inhibitor,

11) a PPAR-γ agonists,

12) a PPAR-δ agonists,

13) an inhibitor of inherent multidrug resistance,

14) an anti-emetic agent,

15) an agent useful in the treatment of anemia,

16) an agent useful in the treatment of neutropenia,

17) an immunologic-enhancing drug,

18) an inhibitor of cell proliferation and survival signaling, and

19) an agent that interfers with a cell cycle checkpoint.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In an embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A4, squalamine, 6-O-chloroacetyl-carbonyl)-fumagillol, thalidomide, angiostatin, troponin-1, or an antibody to VEGF. In an embodiment, the estrogen receptor modulator is tamoxifen or raloxifene.

Also included in the scope of the claims is a method of treating cancer that comprises administering a therapeutically effective amount of a compound designed or selected using the methods disclosed herein in combination with radiation therapy and/or in combination with a compound selected from:

1) an estrogen receptor modulator,

2) an androgen receptor modulator,

3) a retinoid receptor modulator,

4) a cytotoxic/cytostatic agent,

5) an antiproliferative agent,

6) a prenyl-protein transferase inhibitor,

7) an HMG-CoA reductase inhibitor,

8) an HIV protease inhibitor,

9) a reverse transcriptase inhibitor,

10) an angiogenesis inhibitor,

11) PPAR-γ agonists,

12) PPAR-δ agonists,

13) an inhibitor of inherent multidrug resistance,

14) an anti-emetic agent,

15) an agent useful in the treatment of anemia,

16) an agent useful in the treatment of neutropenia,

17) an immunologic-enhancing drug,

18) an inhibitor of cell proliferation and survival signaling, and

19) an agent that interfers with a cell cycle checkpoint.

And yet another embodiment of the invention is a method of treating cancer that comprises administering a therapeutically effective amount of a compound designed or selected using the methods disclosed herein in combination with paclitaxel or trastuzumab.

The invention further encompasses a method of treating or preventing cancer that comprises administering a therapeutically effective amount of a compound designed or selected using the methods disclosed herein in combination with a COX-2 inhibitor.

The instant invention also includes a pharmaceutical composition useful for treating or preventing cancer that comprises a therapeutically effective amount of a compound designed or selected using the methods disclosed herein and a compound selected from:

1) an estrogen receptor modulator,

2) an androgen receptor modulator,

3) a retinoid receptor modulator,

4) a cytotoxic/cytostatic agent,

5) an antiproliferative agent,

6) a prenyl-protein transferase inhibitor,

7) an HMG-CoA reductase inhibitor,

8) an HIV protease inhibitor,

9) a reverse transcriptase inhibitor,

10) an angiogenesis inhibitor, and

11) a PPAR-γ agonist,

12) a PPAR-δ agonists;

13) an inhibitor of cell proliferation and survival signaling, and 14) an agent that interfers with a cell cycle checkpoint.

In each of the aforementioned uses of atomic coordinates of KSP, the coordinates according to Tables 1-4 are preferred.

Additional objects of the present invention will be apparent from the description which follows.

As used herein, the following terms and phrases shall have the meanings set forth below:

Unless otherwise noted, “KSP” includes both native and wild type Kinesin Spindle Protein as well as “KSP analogues”, defined herein as proteins or peptides comprising a ligand binding site substantially as set forth in SEQ ID NO:1. Such KSP analogues include, but are not limited to, a ligand binding site characterized by a three-dimensional structure comprising the relative structural coordinates of amino acid residues set forth in FIG. 10 as set forth in Tables 1-4, ±a root mean square deviation from the conserved backbone atoms of said amino acids of not more than 3.005 Å, more preferably not more than about 2.0 Å, and most preferably not more than about 0.5 Å.

Unless otherwise indicated, “protein” or “molecule” shall include a protein, protein domain, polypeptide or peptide. “Structural coordinates” are the Cartesian coordinates corresponding to an atom's spatial relationship to other atoms in a molecule or molecular complex. Structural coordinates may be obtained using X-ray crystallography techniques or NMR techniques, or may be derived using molecular replacement analysis or homology modeling. Various software programs allow for the graphical representation of a set of structural coordinates to obtain a three-dimensional representation of a molecule or molecular complex. The structural coordinates of the present invention may be modified from the original sets provided in Tables 1-4 by mathematical manipulation, such as by inversion or integer additions or subtractions. As such, it is recognized that the structural coordinates of the present invention are relative, and are in no way specifically limited by the actual x, y, z coordinates of Tables 1-4.

An “agent”, “ligand” or “binding partner” shall include a protein, polypeptide, peptide, nucleic acid, including DNA or RNA, molecule, compound or drug.

“Root mean square deviation” is the square root of the arithmetic mean of the squares of the deviations from the mean, and is a way of expressing deviation or variation from the structural coordinates described herein. The present invention includes all embodiments comprising conservative substitutions of the noted amino acid residues resulting in same structural coordinates within the stated root mean square deviation.

Materials and Methods

Materials and methods provided are intended to assist in a further understanding of the invention and are not to limit the reasonable scope thereof.

Motor Domain of Human KSP, Amino Acids 1-368

MASQPNSSAK KKEEKGKNIQ VVVRCRPFNL AERKASAHSI
VECDPVRKEV SVRTGGLADK SSRKTYTFDM VFGASTKQID
VYRSVVCPIL DEVIMGYNCT IFAYGQTGTG KTFTMEGERS
PNEEYTWEED PLAGIIPRTL HQIFEKLTDN GTEFSVKVSL
LEIYNEELFD LLNPSSDVSE RLQMFDDPRN KRGVIIKGLE
EITVHNKDEV YQILEKGAAK RTTAATLMNA YSSRSHSVFS
VTIHMKETTI DGEELVKIGK LNLVDLAGSE NIGRSGAVDK
RAREAGNINQ SLLTLGRVIT ALVERTPHVP YRESKLTRIL
QDSLGGRTRT SIIATISPAS LNLEETLSTL EYAHRAKNIL
NKPEVNQK

Binding Pocket of Human KSP

Lining the newly formed pocket and surrounding the ligand are amino acid residues:
115 (M), 116(E), 117(G), 118(E), 119(R), 127(W), 130(D), 132(L), 133(A), 134(G), 136(I), 137(P) (from helix-α2 and its insertion loop; residue 116 is at the end of the first portion of helix-α2 and residue 134 is at the beginning of the second portion of helix-α2 thus the insertion loop starts at residue 116 and ends at residue 134);
160(L) (from beta strain-β4);
211(Y), 214(L), 215(E), 217(G), 218(A), 221(R) (from helix-α3); and 239(F) (from beta strain-β6).
   KSP Expression

E. coli cells harboring the KSP (368 residues) vector were grown at 37° C. in LB medium containing 100 μg/ml ampicillin. KSP expression was induced at 25° C. with 0.5mM isopropyl-D(−)-thiogalactopyranoside, and the cells were grown for four additional hours at 25° C. prior to harvest.

Cells from 10 litre were suspended in 75 ml lysis buffer (50 mM PIPES, 2 mM MgCl₂, 1 mM ATP, 1 mM TCEP, 1 mM EGTA, protease inhibitor tablets (one tablet per 50 ml buffer)) and homogenized. Cells were disrupted by passing the homogenized suspension thrice through a Microfluidizer (Model 110-S). The cell lysate was centrifuged at 15,000 rpm for 30 minutes and the supernatant mixed with DE-52 resin (100 ml) pre-equilibrated in SP sepharose Buffer A (50 mM PIPES, 2 mM MgCl₂, 1 mM ATP, 1 mM TCEP, 1 mM EGTA). Supernatant was removed after spinning at 1000 rpm for 10 minutes. Resin was washed twice with one resin volume (100 ml) of 50 mM PIPES, 2 mM MgCl₂, 1 mM ATP, 1 mM TCEP, 1 mM EGTA. The supernatants were pooled and loaded onto SP sepharose column (50 ml, 2.6 cm diameter column, Amersham Biosciences). Kinesin with ˜95% purity was eluted at 0.15 to 0.2 M KCl using 0-30% KCl gradient. The fractions containing KSP (by SDS-PAGE analysis) were pooled and diluted with SP sepharose buffer A to a final KCl concentration of 50 mM. The pool was mixed with 10 ml of High performance Q-sepharose (Amersham Biosciencs) equilibrated in SP sepharose BufferA. The supernatent was collected by spinning at 1000 rpm for 10 minutes. The resin was washed four times with two resin volume. The washes and supernatant were pooled and concentrated on Centriprep-10 to 15 to 17 mg/ml and stored in small alicots at −70° C. The protein was characterized by N-terminal sequence analysis by Edman degradation on an Applied Biosystem model 470A gas phase sequencer. Protein concentration was determined with quantitative amino acid analysis by using a post column ninhydrin derivatization method on a Beckman 6300 analyzer. Molecular weight was determined on Deca-LCQ (Finnegan) mass spectrometer. Molar mass and size distribution was determined by multi-angle light scattering detector (Wyatt technology, DAWN EOS) connected to size exclusion column on Millenium HPLC.

Crystallization

The concentrated kinesin (ADP, Mg++) protein at about 15 mg/ml in 50 mM PIPES buffer at pH 6.8 in the presence of 2 mM MgCl₂, 1 mM TECP, 1 mM ATP, 84 mM KCl, and 1 mM EGTA was incubated with 1 mM inhibitor Compound 5-2b ((+)-monastrol). Small single crystal seeds were obtained by hanging drop method with well solution containing 20% PEG3350, 0.15M K₂HPO₄ and 0.1M HEPES buffer at pH7.0 in about four days. Crystals suitable for X-ray data collection were obtained by macro-seeding in hanging drops with well solution containing 14% PEG3350, 0.2M K₂HPO₄ and 0.1M HEPES at pH 6.8 in about two weeks. Hanging drops were formed by equal volume of protein and well solutions.

X-Ray Data Collection and Procession

The X-ray diffraction data at 2.5 Å resolution were collected at 100K at synchrotron beamline 17-ID of the Advanced Photon Source at Argonne National Laboratory. Prior to data collection the crystal was soaked in the cryo-protectant solution for 20 minutes that contains 20% PEG3350, 0.15M K₂BPO₄, 20% PEG200, and 0.1M HEPES buffer at pH6.8. The crystal was then frozen in liquid nitrogen. The X-ray wavelength was set to 1 Å. The data were collected at 0.20 oscillation per frame with 1000 frames total and 1 second exposure per frame at 250 mm detector to crystal distance. The data were processed and scaled by use of HKL2000 package. The crystal is in orthorhombic space group of P2₁2₁2₁ with cell dimensions of a=69.5 Å b=79.5 Å and c=159.0 Å. The completeness of the data set was 99%. The Rsym was 0.084.

Structure Determination and Refinement

The structure was determined by the use of the molecular replacement method in cooperation with extensive model rebuilding and dynamic refinement. The kinesin protein coordinates in the binary complex crystal structure of kinesin bound with ADP (Mg++) was used as the search model. The molecular replacement solution was obtained with use of program AmoRe at 4.0 Å to 15 Å resolution range, which gave R-factor of 0.48 and correlation coefficient of 0.60. The initial protein model was rebuilt and refined literally at 2.5 Å resolution, those included dynamic refinement, energy minimization and temperature factor refinement. The Compound 5-2b density became apparent at the fourth rebuilding and refinement cycle. Finally, 441 water molecules were added in the model and the R-factor was 0.21 with R-free of 0.26 with good geometry (RMSD_{bonds}=0.007 Å, RMSD_{angles}=1.32°). The current protein model binds with one ADP, one Mg++ ion and one Compound 5-2b. It starts at residue Asn18 to Lys362 with a gap from residue Asn271 to Asn287 (missing loop 11 from Ile272 to Gly286) due to lack of electron density. There are two complexes in an asymmetric unit.

Tertiary Structure of KSP/ADP/Compound 5-2b

The 3-dimensional, tertiary structure of KSP, bound with Mg-ADP and Compound 5-2b ((+)-monastrol), was determined at 2.5 Å resolution with use of phases derived from a combination of molecular replacement, extensive manual rebuilding, and dynamic refinement. Two identical protein complexes were found in the asymmetric unit of the crystal and were related by a local, non-crystallographic 2-fold axis. For each, the electron density of the protein as well as those-of the ligands (ADP, Mg++, and Compound 5-2b) was all well defined. Compound 5-2b was seen to be of the S handedness. Residues 2-17, 272-286,and 363-368 were disordered and showed no electron densities (The N-terminal Metl residue was processed upon expression). See FIGS. 1-8.

Fluorescence of Trp127 of KSP(368)-ADP −/+Inhibitors

Materials

2× kinesin buffer: 160 mM K-Hepes, 2 mM MgCl₂, 2 mM EGTA, 2 mM DTT (added fresh daily), and 100 mM KCl, pH 6.8.
Nucleotide: nucleotide is resuspended to 200 mM in 50 mM K-Hepes (pH 6.8).
Nucleotide is diluted 1:1 with 200 MM MgCl₂ to a stock concentration of 100 mM of 1:1 nucleotide:MgCl₂.
Cuvette volume=300 μl
   Methods
1) Add 281 μl of 1× kinesin buffer, ±nucleotide, and H₂O (Nucleotide=none, 1 mM AMPPNP, or 1 mM ADP (final concentration)).
2) Add 18.75 μl of 4 μM stock nucleotide-free KSP(367H).
3) Add compound sequentially from DMSO stock (with all the volume of all additions ≦0.6 μl).
4) Measure fluorescence after each addition (starting with buffer only).
5) Example titration for Compound 8-1 with KSP(367H)ADP: 281 μl of 1× kinesin buffer+1 mM ADP:
add 250 nM KSP (18.75 μl of 4 uM nucleotide-free stock)
add 1 nM Compound 8-1 (1 nM_{f}) (addition of 0.3 μl of 0.001 mM stock)
add 2 nM Compound 8-1 (3 nM_{f}) (addition of 0.6 μl of 0.001 mM stock)
add 4 nM Compound 8-1 (7 nM_{f}) (addition of 0.12 μl of 0.01 mM stock)
add 3 nM Compound 8-1 (10 nM_{f}) (addition of 0.09 μl of 0.01 mM stock)
add 20 nM Compound 8-1 (30 nM_{f}) (addition of 0.6 μl of 0.01 mM stock)
add 40 nM Compound 8-1 (70 nM_{f}) (addition of 0.12 μl of 0.1 mM stock)
add 30 nM Compound 8-1 (100 nM_{f}) (addition of 0.09 μl of 0.1 mM stock)
add 200 nM Compound 8-1 (300 nM_{f}) (addition of 0.6 μl of 0.1 mM stock)
add 400 nM Compound 8-1 (700 nM_{f}) (addition of 0.12 μl of 1 mM stock)
add 300 nM Compound 8-1 (1000 nM_{f}) (addition of 0.09 μl of 1 mM stock)
add 2000 nM Compound 8-1 (3000 nM_{f}) (addition of 0.6 μl of 1 mM stock).
6) After each addition, measure steady-state fluorescence under the following conditions:
λₑₓ=388 nm, λₑₘ=342-346 nm, band width=3 nm ex/3 nm em, wavelength increment=0.5 nm, integration time=2 s.
7) Repeat the same titration series:
in the absence of KSP (to determine compound-related background), and in the absence of KSP, but in the presence of 1 μM L-tryptophan (to determine compound-related effects on the amino acid itself).
   Calculations

At the peak emission wavelength for W127 in KSP(367H) (=344 nm) measure the compound emission in kinesin buffer as a function of [compound]; measure fluorescence of L-tryptophan as a function of [compound]; measure fluorescence of KSP(367H) as a function of [compound]; correct KSP(367H) fluorescence for its decrease over time (due to losses of protein to the cuvette); subtract compound emission from L-tryptophan emission; subtract compound emission from KSP(367H) emission. Calculate the fraction of fluorescence of L-tryptophan vs [compound]: (L-trp fluorescence (344 nm) at given [compound])/(L-trp fluorescence (344 nm) at 0 cpd); calculate the fraction of fluorescence of KSP(367H) vs [compound]: (KSP fluorescence (344 nm) at given [compound])/(KSP fluorescence (344 nm) at 0 cpd); then normalize: KSP (frcn fl)/L-trp(frcn fl) and plot vs [compound].

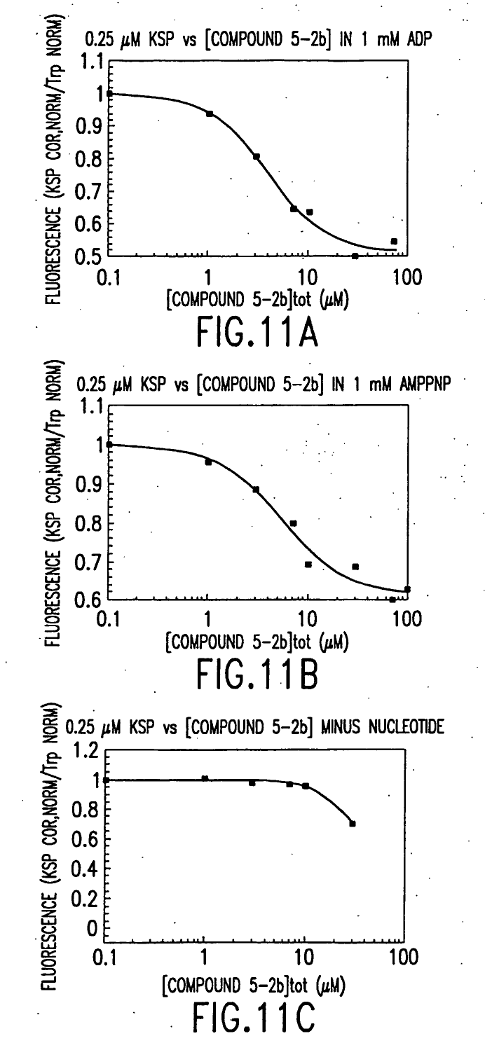

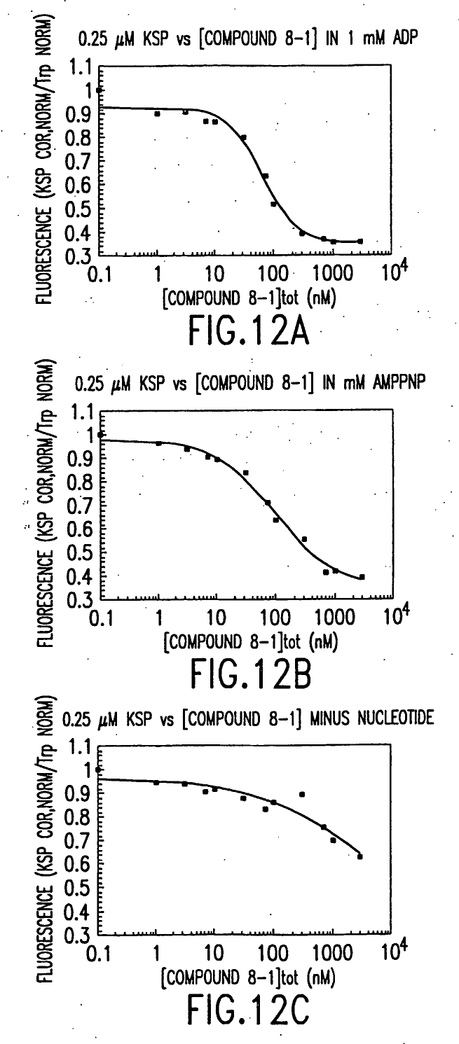

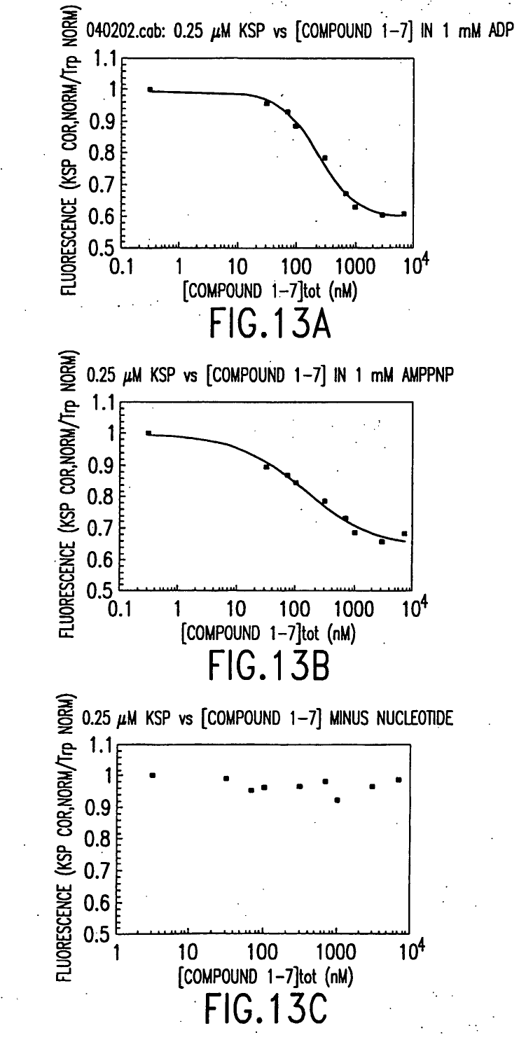

Results of this assay are illustrated in FIGS. 11-13.

Compounds that were utilized in the identification and testing of the novel KSP binding site that is disclosed herein may be prepared by the methods described below: 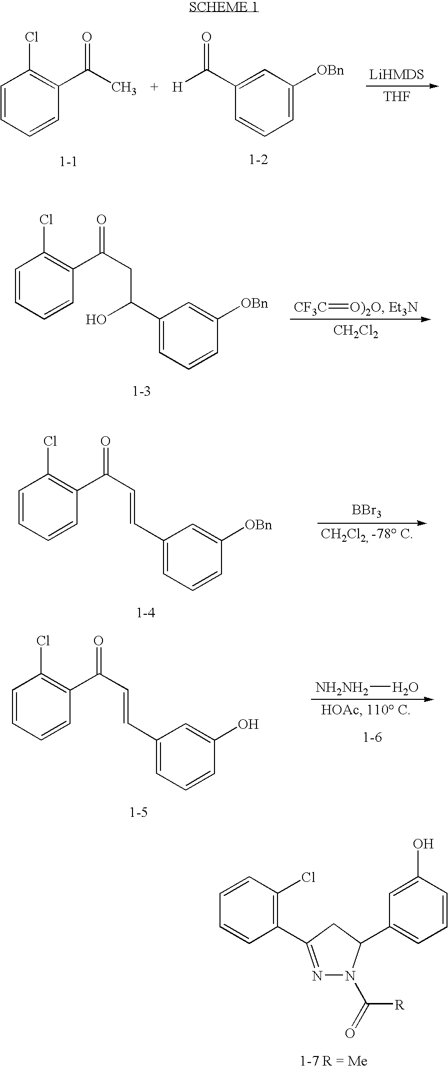
Step 1:

### 3-[3-(benzyloxy)phenyl]-1-(2-chlorophenyl)prop-2-en-1-one (1-4)

To a solution of 2′-chloroacetophenone (1-1) (1.26 mL, 9.70 mmol) in 40 mL of THF at −78° C. was slowly added 10.7 mL (10.7 mmol) of a 1M LiHMDS solution in THF. After stirring for 1 h at −78° C., a solution of 2.05 g (9.70 mmol) of 3-benzyloxy-benzaldehyde (1-2) in 8 mL of THF was added, and stirring was continued at that temperature for an additional hour. The mixture was then dumped into a separatory funnel containing 100 mL of saturated aqueous NH₄Cl and extracted twice with 100 mL of EtOAc. The organic phases were combined, washed with 100 mL of brine, and dried over Na₂SO₄. After filtering off the drying agent, the solvent was removed on a rotary evaporator, and the residue was dissolved in 50 mL of CH₂Cl₂. After cooling to −78° C., 4 mL of triethylamine and 2 mL of trifluoroacetic anhydride were added sequentially, and the mixture was allowed to warm to rt and stir for 12 h. The reaction was then dumped into a separatory funnel with 100 mL of 1M HCl, the layers were separated, and the aqueous phase extracted again with CH₂Cl₂. The organic layers were combined, washed again with 1 M HCl, washed with water, and dried over Na₂SO₄. After concentration, the crude material was purified by chromatography on silica gel with a gradient of 0 to 40% EtOAc in hexanes over 45 min to provide 1-4 as a viscous yellow oil. Data for 1-4: ¹H NMR (500 MHz, CDCl₃) δ7.5-7.0 (m, 15H) 5.1 (s, 2H) ppm.

Step 2:

### 1-(2-chlorophenyl)-3-(hydroxyphenyl)prop-2-en-1-one (1-5)

To a solution of 740 mg (2.12 mmol) of 1-4 in 15 mL of CH₂Cl₂ at −78° C. was added dropwise 2.75 mL (2.75 mmol) of a 1M solution of BBr₃ in CH₂Cl₂. After stirring for 30 min at that temperature, 1 mL of MeOH was added, and the mixture was dumped into water, extracted twice with 50 mL of CH₂Cl₂, washed again with water, and dried over Na₂SO₄. After concentration, the residue was purified by column chromatography on silica gel with a gradient of 2 to 70% EtOAc in hexanes over 30 min to provide 1-5 as a beige solid. Data for 1-5: ¹H NMR (500 MHz, CDCl₃) δ 7.5-7.3 (m, 5H), 7.25 (m, 1H), 7.2-7.0 (m, 3H), 6.9 (m, 1H), 5.1 (bs, 1H) ppm.

Step 3:

### 3-[1-acetyl-3-(2-chlorophenyl)-4,5-dihydro-1H-pyrazol-5-yl]phenol (1-7)

To a solution of 120 mg (0.46 mmol) of chalcone 1-5 in 4 mL of acetic acid was added 50 μL (0.93 mmol) of hydrazine hydrate. The reaction was then placed in an oil bath at 110° C. for 24 h. After cooling to rt, the solvents were removed on a rotary evaporator, the residue was dissolved in 50 mL of CH₂Cl₂, washed twice with aqueous NaHCO₃, dried over Na₂SO₄, and concentrated. The residue was then purified by column chromatography on silica gel with a gradient of 5 to 75% EtOAc in hexanes over 30 min to provide 1-7 as a fluffy white solid. Data for 1-7: ¹H NMR (500 MHz, CDCl₃) δ 7.75 (m, 1H), 7.45 (m 1H), 7.4-7.3 (m, 2H), 7.2 (m, 1H), 6.8 (d, 1H), 6.7 (m, 2H), 5.5 (m, 1H), 3.9 (m, 1H), 3.3 (m, 1H), 2.4 (s 3H) ppm. HRMS (ES) calc'd M+H for C₁₇H₁₅ClN₂O₂: 315.0895. Found: 315.0904. 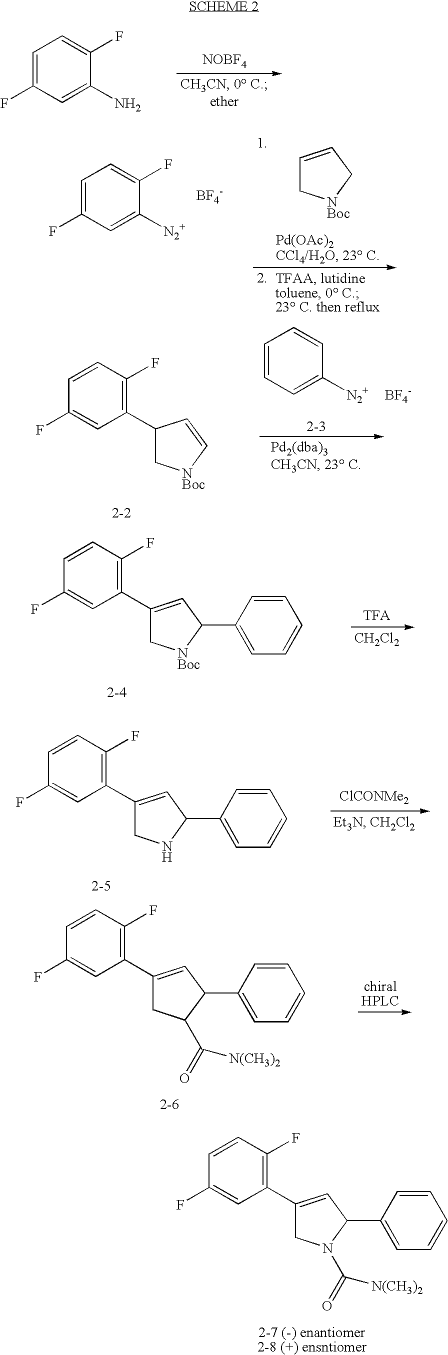
Step 1:

### 2.5-difluorobenzenediazonium tetrafluoroborate (2-1)

Nitrosonium tetrafluoroborate (905 mg, 7.75 mmol, 1.00 equiv) was added to a solution of 2,5-difluoroaniline (0.780 mL, 7.75 mmol, 1 equiv) in acetonitrile (50 mL) at 0° C. The resulting mixture was stirred for 1 h, then diluted with ethyl ether (150 mL). The precipitate was filtered and air-dried to give 2,5-difluorobenzenediazonium tetrafluoroborate (2-1) as a tan solid. ¹H NMR (300 MHz, CD₃OD) δ 8.54 (m, 1H), 8.24 (m, 1H), 7.95 (m, 1H).

Step 2:

### tert-butyl 3-(2,5-difluorophenyl)-2,3-dihydro-1H-pyrrole-1-carboxylate (2-2)

Palladium(II) acetate (67 mg, 0.30 mmol, 0.020 equiv) was added to a vigourously stirred, deoxygenated mixture of tert-butyl 2,5-dihydro-1H-pyrrole-1-carboxylate (2.59 mL, 15.0 mmol, 1 equiv) and 2,5-difluorobenzenediazonium tetrafluoroborate (2-1, 3.42 g, 15.0 mmol, 1.00 equiv) in water and carbon tetrachloride (1:1, 150 mL) at 23° C., and the resulting mixture was stirred for 20 h. The reaction mixture was concentrated, and the residue partitioned between ethyl acetate (300 mL) and saturated aqueous sodium bicarbonate solution (75 mL). The organic layer was washed with brine, then dried over sodium sulfate and concentrated. The residue was dissolved in toluene (200 mL), and the resulting solution concentrated in vacuo to facilitate azeotropic removal of residual water. 2,6-Lutidine (3.50 mL, 30.0 mmol, 2.00 equiv) and trifluoroacetic anhydride (1.48 mL, 10.5 mmol, 0.700 equiv) were then sequentially added to a solution of the residue in toluene (100 mL) at −10° C. The resulting mixture was allowed to warm to 10° C. over 16 h, then heated at reflux for 1 h. The reaction mixture was allowed to cool to 23° C., then concentrated. The residue was partitioned between ethyl acetate (300 mL) and saturated aqueous sodium bicarbonate solution (150 mL). The organic layer was dried over sodium sulfate and concentrated. The residue was purified by flash column chromatography (hexanes initially, grading to 20% EtOAc in hexanes) to give tert-butyl 3-(2,5-difluorophenyl)-2,3-dihydro-1H-pyrrole-1-carboxylate (2-2) as a red oil. ¹H NMR (500 MHz, CDCl₃) major rotamer: δ 7.03-6.84 (m, 3H), 6.70 (br s, 1H), 5.01 (br s, 1H), 4.42 (m, 1H), 4.13 (m, 1H), 3.60 (m, 1H), 1.50 (s, 9H).

Step 3:

### tert-butyl 4-(2,5-difluorophenyl)-2-phenyl-2,5-dihydro-1H-pyrrole-1-carboxylate (2-4)

Tris(dibenzylideneacetone)dipalladium(0) (59 mg, 064 mmol, 0.020 equiv) was added to a deoxygenated mixture of tert-butyl 3-(2,5-difluorophenyl)-2,3-dihydro-1H-pyrrole-1-carboxylate (2-2,900 mg, 3.20 mmol, 1 equiv), benzenediazonium tetrafluoroborate (1-3, prepared by the method described above for 2-3, 614 mg, 3.20 mmol, 1.00 equiv), and sodium acetate trihydrate (1.32 g, 9.60 mmol, 3.00 equiv) in acetonitrile (70 mL) at 23° C. The reaction mixture was stirred for 16 h, then partitioned between saturated aqueous sodium bicarbonate solution and ethyl acetate (2×70 mL). The combined organic layers were dried over sodium sulfate and concentrated. The residue was purified by flash column chromatography (hexanes initially, grading to 40% hexanes in EtOAc) to provide tert-butyl 4-(2,5-difluorophenyl)-2-phenyl-2,5-dihydro-1H-pyrrole-1-carboxylate (2-4) as an orange oil. LRMS m/z (M+H—CH₃) 343.0 found, 343.1 required.

Step 4:

### 4-(2,5-difluorophenyl)-2-phenyl-2,5-dihydro-1H-pyrrole (2-5)

Trifluoroacetic acid (20 mL) was added to a solution of tert-butyl 4-(2,5-difluorophenyl)-2-phenyl-2,5-dihydro-1H-pyrrole-1-carboxylate (2-4, 700 mg, 1.96 mmol, 1 equiv) in dichloromethane (50 mL) at 23° C., and the resulting mixture was stirred for 30 min, then concentrated to give 4-(2,5-difluorophenyl)-2-phenyl-2,5-dihydro-1H-pyrrole (2-5) as a TFA salt (brown oil). LRMS m/z (M+H) 258.1 found, 258.1 required.

Step 5:

### 4-(2,5-difluorophenyl)-N,N-dimethyl-2-phenyl-2,5-dihydro-1H-pyrrole-1-carboxamide (2-6)

Triethylamine (1.37 mL, 9.79 mmol, 5.00 equiv) and dimethylcarbamoyl chloride (0.180 mL, 1.96 mmol, 1.00 equiv) were added to a solution of 4-(2,5-difluorophenyl)-2-phenyl-2,5-dihydro-1H-pyrrole (2-5, 1.96 mmol) in dichloromethane (50 mL) at 23° C., and the resulting mixture was stirred for 2 h, then concentrated. The residue was partitioned between saturated aqueous sodium bicarbonate solution (75 ml) and ethyl acetate (100 mL). The organic layer was dried over sodium sulfate and concentrated. The residue was purified by reverse-phase LC (H₂O/CH₃CN gradient w/0.1% TFA present) to provide 4-(2,5-difluorophenyl)-N,N-dimethyl-2-phenyl-2,5-dihydro-1H-pyrrole-1-carboxamide (2-6) as an off-white solid. ¹H NMR (500 MHz, CDCl₃) δ 7.35-7.29 (m, 4H), 7.25 (m, 1H), 7.05 (m, 1H), 7.00 (m, 1H), 6.96 (m, 1H), 6.40 (br s, 1H), 6.13 (m, 1H), 4.88 (ddd, 1H, J=13.7, 5.6, 2.0 Hz), 4.52 (d, 1H, J=13.7 Hz), 2.88 (s, 6H). LRMS m/z (M+H) 329.1 found, 329.1 required.

Step 6:

### Enantiomers of 4-(2,5-difluorophenyl)-N,N-dimethyl-2-phenyl-2,5-dihydro-1H-pyrrole-1-carboxamide (2-7 and 2-8)

Resolution of enantiomers of racemic 4-(2,5-difluorophenyl)-N,N-dimethyl-2-phenyl-2,5-dihydro-1H-pyrrole-1-carboxamide (2-6) by chiral normal-phase HPLC (Chiralcel OD column: 0.1% diethylamine in 40% ethanol in hexanes) provided in order of elution 2-7 (−) and 2-8 (+). 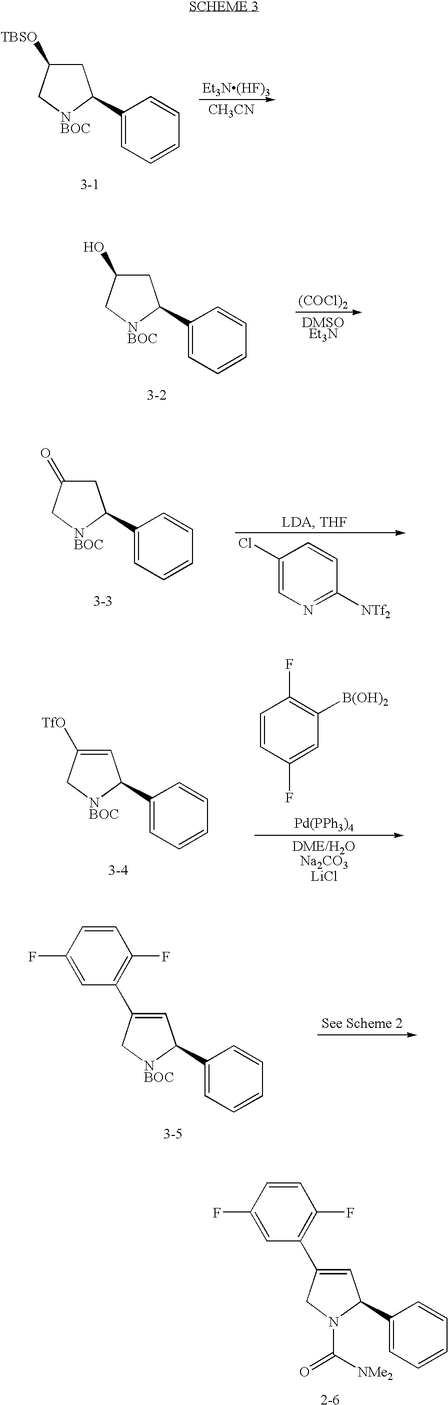
Step 1:

### (2S,4S)-tert-Butyl 4-hydroxy-2-phenylpyrrolidine-1-carboxylate (3-2)

To a flame dried flask equipped with stir bar was added tert-butyl (2S,4S)-4-{[tert-butyl(dimethyl)silyl]oxy}-2-phenylpyrrolidine-1-carboxylate (3-1, prepared from (S)-(−)4-chloro-3-hydroxybutyronotrile by the method of Maeda, et al Synlett 2001, 1808-1810, 7.8 g, 20.7 mmol) and anhydrous acetonitrile (20.0 mL). The resulting solution was treated with triethylamine trihydrofluoride (10.1 mL, 62.0 mmol) while stirring under N₂. The reaction stirred 12 h at 40° C. The reaction was then diluted with EtOAc (100 mL) and poured into 5% aq. NaHCO₃. Following cessation of gas evolution, the organic layer was washed three addition times with 5% aq. NaHCO₃. The organic layer was dried over magnesium sulfate, filtered and concentrated to provide crude product. Recrystallization was effected from EtOAc/hexanes to provide (2S,4S)-tert-butyl 4-hydroxy-2-phenylpyrrolidine-1-carboxylate (3-2) as a white crystalline solid. ¹H NMR (300 MHz, CDCl₃) rotamers δ 7.38-7.18 (m, 5H), 4.90 (m, 1H), 4.42 (m, 1H), 3.88 (m, 1H), 3.56 (dd, J=11.5, 4.0 Hz, 1H), 2.60 (m, 1H), 2.03 (m, 1H), 1.50 and 1.20 (br s, 9H); MS 208.0 found, 208.1 (M−C(CH₃)₃) required.

Step 2:

### (2S)-tert-butyl 4-oxo-2-phenylpyrrolidine-1-carboxylate (3-3)

To a flame dried flask equipped with stir bar was added 150 mL anhydrous dichloromethane which was cooled to −78° C. Oxalyl chloride (3.8 mL, 44 mmol) and DMSO (4.8 mL, 61 mmol) were added sequentially and the reaction stirred for 10 min. (2S,4S)-tert-butyl 4-hydroxy-2-phenylpyrrolidine-1-carboxylate (3-2, 2.28 g, 8.73 mmol) in 10 mL anhydrous dichloromethane was added dropwise and stirred 1 h at −78° C. Triethylamine (12 mL, 87 mmol) was added and the reaction was warmed to 0° C. over 1 h. Upon completion, the reaction was washed with 5% NaHCO₃, brine and dried over MgSO₄. The organic layer was concentrated to provide crude (2S)-tert-butyl 4-oxo-2-phenylpyrrolidine-1-carboxylate (3-3). Recrystallization was effected with EtOAc/hexanes. ¹H NMR (300 MHz, CDCl₃) δ 7.35 (m, 3H), 7.17 (m, 2H), 5.38 (m, 1H), 4.08 (d, J=19.5 Hz, 1H), 3.90 (d, J=19.3 Hz, 1H), 3.13 (dd, J=18.8, 9.8 Hz, 1H), 2.58 (dd, J=18.6, 2.4 Hz, 1H), 1.40 (br s, 9H); MS 206.0 found, 206.1 (M−C(CH₃)₃) required.

Step 3:

### (2S)-tert-butyl 2-phenyl-4-{[(trifluoromethyl)sulfonyl]oxy}-2,5-dihydro-1H-pyrrole-1-carboxylate (3-4)

To a flame dried flask equipped with stir bar was added ketone (2S)-tert-butyl 4-oxo-2-phenylpyrrolidine-1-carboxylate (3-3, 0.16 g, 0.62 mmol) and anhydrous THF (2 mL). The resulting solution was cooled to −78° C., and treated dropwise with lithium hexamethyldisilylamide (LHMDS, 0.68 mL, 1M in THF, 0.68 mmoL). The reaction stirred 1 h at −78° C., and N-(5-chloropyridin-2-yl)-1,1,1-trifluoro-N-[(trifluoromethyl)sulfonyl]-methanesulfonamide (0.27 g, 068 mmol) was added neat in one portion. The reaction was allowed to warn to 0° C. and stirred 4 hours total. The reaction was diluted with Et2O (10 mL) and washed successively with H₂O (10 mL) and brine (10 mL). The organic layer was dried over MgSO₄, filtered and concentrated. The crude residue was purified by flash column choromatography (0-20% EtOAc/hexanes gradient, 15 min) to provide (2S)-tert-butyl 2-phenyl-4-{[(trifluoromethyl)sulfonyl]oxy}-2,5-dihydro-1H-pyrrole-1-carboxylate (3-4). ¹H NMR (300 MHz, CDCl₃) major rotamer: δ 7.30 (m, 5H), 5.72 (m, 1H), 5.48 (m, 1H), 4.42 (m, 2H), 1.18 (s, 9H); MS 379.0 found 379.1 (M−CH₃) required.

Step 4:

### (2S)4-(2,5-difluorophenyl)-2-phenyl-N,N-dimethyl-2,5-dihydro-1H-pyrrole-1-carboxamide (3-5)

To a flame dried flask equipped with stir bar was added (2S)-tert-butyl 2-phenyl-4-{[(trifluoromethyl)sulfonyl]oxy}-2,5-dihydro-1H-pyrrole-1-carboxylate (3-4, 0.250 g, 0.636 mmol), 2,5-difluorophenyl boronic acid (0.251 g, 1.59 mmol), Na₂CO₃ (0.202 g, 1.91 mmol), and LiCl (0.081 g, 1.91 mmol). The solids were dissolved in 20 mL 4:1 DME/H₂O and degassed with nitrogen. Pd(PPh₃)₄ (0.037 g, 0.032 mmol) was added and the reaction was sealed under nitrogen and heated to 90° C. for 2 h. Upon completion, the reaction was partitioned between 5% aq. NaHCO₃ and EtOAc (3×50 mL), and the combined organic layers were dried over MgSO₄. Following filtration, the organic layer was concentrated and purified via flash column chromatography (SiO₂, 0-20% EtOAc/hexanes gradient) to provide (2S)-tert-butyl 4-(2,5-difluorophenyl)-2-phenyl-2,5-dihydro-1H-pyrrole-1-carboxylate (3-5). Further transformations followed those described in Scheme 1 to provide the instant compound 2-6.

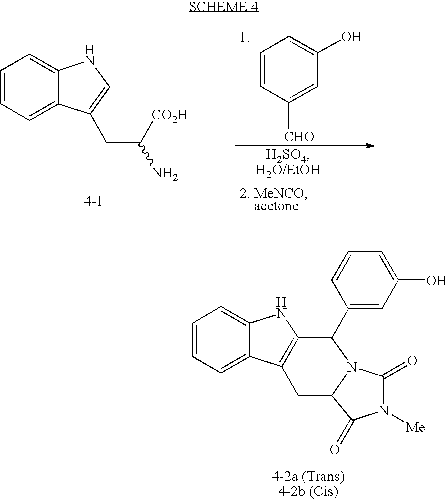

### Trans-1H-Imidazo[1′, 5′:1,6]pyrido[3,4-b]indole-1,3(2H)-dione,5,6,11,11a-tetrahydro-2-methyl-5-(3-hydroxyphenyl) (4-2a)

To a mixture of DL-tryptophan (1.5 g, 7.44 mmol), 3-hydroxybenzaldehyde (0.90, 7.44 mmol) in EtOH (3 mL) was added aq. H₂SO₄ (14.9 mL of a 0.5 M solution). The reaction was heated to 50 C for 12 h. The reaction mixture was partly concentrated to remove EtOH and resuspended in H₂O (5 mL). The precipitate was collected by filtration and dried in vacuo. The portion of this solid residue (0.14 g, 0.47 mmol) was dissolved in acetone (3 mL) and treated with methyl isocyanate. The reaction mixture was heated at 150 C. in a sealed vessel for 15 min in a microwave reactor. The reaction was cooled to r.t. and concentrated. The residue was absorbed onto silica gel then purified on an ISCO automated system affixed with a Biotage flash 40(s) cartridge eluting with 0-100% EtOAc in hexane at 20 mL/min over 30 min to afford a mixture of 4-2a/4-2b Trituration of this mixture with diethyl ether provided pure 4-2a. Data for 4-2a: ¹H NMR (600 MHz, CD₃OD) δ 7.52 (d, J=8 hz, 1H), 7.27 (d, J=8 hz, 1H), 7.18 (m, 1H), 7.12 (m, 1H), 7.07 (m, 1H), 6.84 (m, 1H), 6.74 (m, 2H), 6.24 (s, 1H), 4.44 (m, 1H), 3.43 (m, 1H), 3.01 (s, 3H), 2.88 (m, 1H) ppm. HRMS Calcd (M+1) 348.1270; found 348.1343.

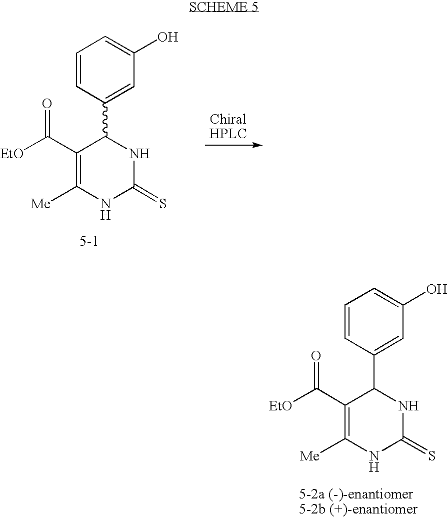

### (−)4-(3-Hydroxyphenyl)-6-methyl-2-thioxo-1,2,3,4-tetrahydro-4H-pyrimidin-5-carboxylic acid ethyl ester (5-2a) and (+)-4-(3-Hydroxyphenyl)-6-methyl-2-thioxo-1,2,3,4-tetrahydro-4H-pyrimidin-5-carboxylic acid ethyl ester (5-2b)

Racemic monastrol (50 mg, Tocris) was resolved by chiral HPLC (Chiralpak AD column 5×50 cm; 20% EtOH/80% (hexanes +0.1% diethylamine); flow=60 mL/min) to yield (−)-enantiomer 1-2A (R_{T}=57.0 min) and (+)-enantiomer 5-2 B (R_{T}=71.2 min). Enantiomer 5-2B was crystallized from hexanes to yield a yellow solid.

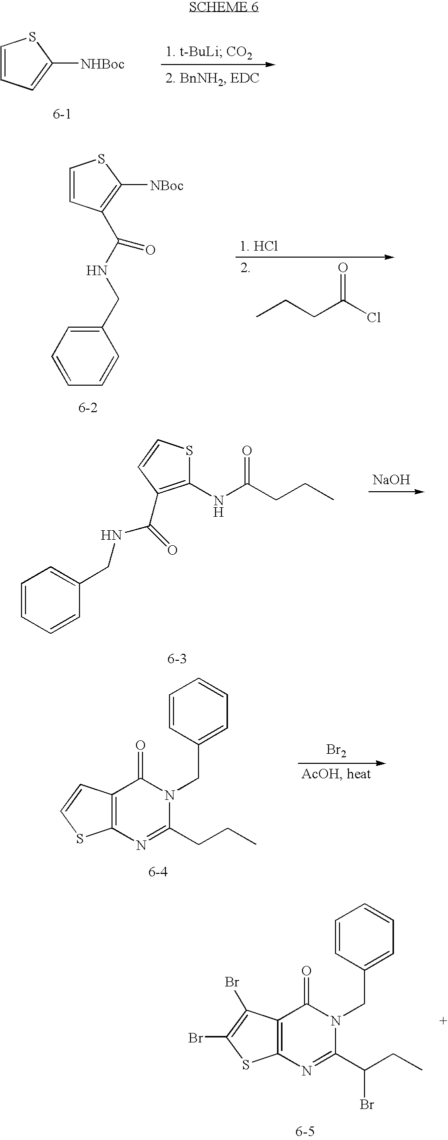

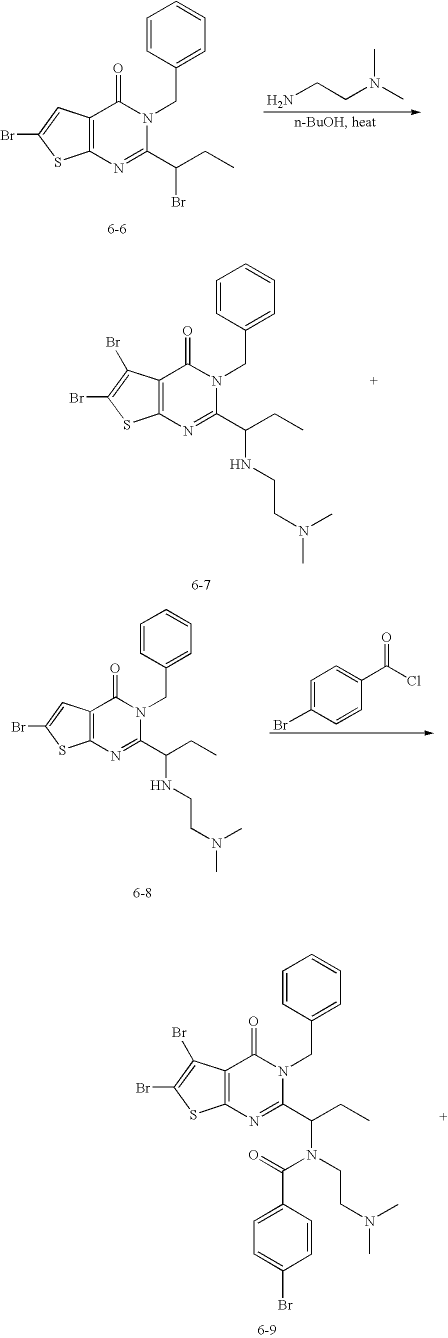

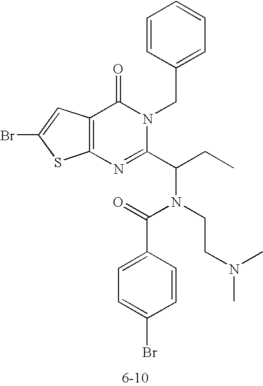

### tert-Butyl 3-[(benzylamino)carbonyl]thien-2-ylcarbamate (6-2)

A solution of tert-butyllithium in pentane (1.7 M, 42.5 mL, 72.3 mmol, 2.40 equiv) was added to a solution of tert-butyl thien-2-ylcarbamate (6-1, 6.00 g, 30.1 mmol, 1 equiv) in THF (300 mL) at −78° C. The reaction mixture was stirred for 45 min, then solid CO₂ (approximately 20 g) was added and the resulting mixture was warmed to 0° C. and stirred for 30 minutes. The reaction mixture was partitioned between aqueous 1 N hydrochloric acid solution and ethyl acetate (2×150 mL). The combined organic layers were dried over sodium sulfate and concentrated. The residue was purified by flash column chromatography (hexanes initially, grading to 100% ethyl acetate), and the polar fractions were concentrated. A solution of the residue, benzylamine (6.61 g, 61.7 mmol, 2.05 equiv), 1-(3-dimethylaminopropyl)-3-ethylcarbodiimide hydrochloride (5.91 g, 30.8 mmol, 1.02 equiv), 1-hydroxy-7-azabenzotriazole (4.19 g, 30.8 mmol, 1.02 equiv), and triethylamine (8.59 mL, 61.7 mmol, 2.05 equiv) in DMF (100 mL) was stirred at 55° C. for 24 h. The reaction mixture was concentrated, and the residue was partitioned between saturated aqueous sodium bicarbonate solution and ethyl acetate (3×100 mL). The combined organic layers were dried over sodium sulfate and concentrated. The residue was purified by flash column (hexanes initially, grading to 100% ethyl acetate) to give tert-butyl 3-[(benzylamino)carbonyl]thien-2-ylcarbamate (6-2) as a colorless oil. ¹H NMR (300 MHz, CDCl₃) δ 7.37 (m, 5H), 6.87 (d, 1H, J=5.8 Hz), 6.69 (d, 1H, J=5.8 Hz), 6.13 (s, 1H), 4.61 (d, 2H, J=5.5 Hz), 1.52 (s, 9H).

### N-benzyl-2-(butyrylamino)thiophene-3-carboxamide (6-3)

A solution of tert-butyl 3-[(benzylamino)carbonyl]thien-2-ylcarbamate (6-2, 500 mg, 1.50 mmol, 1 equiv) was saturated with HCl gas at 0° C., and the resulting solution was stirred at 0° C. for 1 h, then allowed to warm to 23° C. and stirred for 1 h. The reaction mixture was concentrated and the residue was dissolved in pyridine (10 mL). The resulting solution was cooled to 0° C., and butyryl chloride (420 μL, 4.04 mmol, 2.69 equiv) was added in three equal portions over 1 h. The reaction mixture was partitioned between aqueous sodium bicarbonate solution and ethyl acetate (50 mL). The organic layer was dried over sodium sulfate and concentrated. The residue was purified by flash column (hexanes initially, grading to 100% ethyl acetate) to give N-benzyl-2-(butyrylamino)thiophene-3-carboxamide (6-3) as an off-white solid. ¹H NMR (300 MHz, CDCl₃) δ 7.36 (m, 5H), 6.92 (d, 1H, J=6.1 Hz), 6.76 (d, 1H, J=5.8 Hz), 6.23 (s, 1H), 4.62 (d, 2H, J=5.8 Hz), 2.47 (t, 2H, J=7.3 Hz), 1.80 (sextet, 2H, J=7.3 Hz), 1.01 (t, 3H, J=7.3 Hz).

### 3-benzyl-2-propylthieno[2,3-d]pyrimidin-4(3H)-one (6-4)

A mixture of N-benzyl-2-(butyrylamino)thiophene-3-carboxamide (6-3, 230 mg, 0.76 mmol, 1 equiv) and sodium hydroxide (3 mg, 0.08 mmol, 0.1 equiv) in ethylene glycol (5 mL) was heated at 130° C. for 5 h. The reaction mixture was allowed to cool, then partitioned between a half-saturated aqueous sodium chloride solution and ethyl acetate (2×75 mL). The combined organic layers were dried over sodium sulfate and concentrated. The residue was purified by flash column (hexanes initially, grading to 100% ethyl acetate) to provide 3-benzyl-2-propylthieno[2,3-d]pyrimidin-4(3H)-one (6-5) as a colorless oil which solidified upon standing. ¹H NMR (300 MHz, CDCl₃) δ 7.48 (d, 1H, J=5.8 Hz), 7.31 (m, 3H), 7.19 (d, 1H, J=5.8 Hz), 7.17 (d, 2H, J=7.9 Hz), 5.42 (s, 2H), 2.72 (t, 2H, J=7.6 Hz), 1.78 (sextet, 2H, J=7.6 Hz), 0.97 (t, 3H, J=7.3 Hz).

### 3-benzyl-5,6-dibromo-2-(1-bromopropyl)thieno[2,3-d]pyrimidin-4(3H)-one (6-5) and 3-benzyl-6-bromo-2-(1-bromopropyl)thieno[2,3-d]pyrimidin-4(3H)-one (6-6)

A solution of 3-benzyl-2-propylthieno[2,3-d]pyrimidin-4(3H)-one (6-4, 100 mg, 0.35 mmol, 1 equiv), potassium acetate (207 mg, 2.1 mmol, 6 equiv) and bromine (338 mg, 2.1 mmol, 6 equiv) in acetic acid (2 mL) was heated at 100° C. for 3 hr. The reaction was concentrated, and the residue was purified by flash chromatography. Elution with 30% hexanes/EtOAc gave 3-benzyl-5,6-dibromo-2-(1-bromopropyl)thieno[2,3-d]pyrimidin-4(3H)-one (6-5) as a colorless solid. ¹H NMR (500 MHz, CDCl₃) δ 7.30 (m, 1H), 7.14 (d, J=7.3 Hz, 2H), 6.19 (d, J=16.3 Hz, 1H), 4.87 (d, J=16.3 Hz, 1H), 4.62 (t, J=7.3Hz, 1H), 2.35 (m, 1H), 2.18 (m, J=1H), 0.72 (t, J=7.3 Hz, 3H). Further elution with the same eluant gave 3-benzyl-6-bromo-2-(1-bromopropyl)thieno[2,3-d]pyrimidin-4(3H)-one (2-6) as a colorless gum. ¹H NMR (500 MHz, CDCl₃) δ 7.53 (s, 1H), 7.34 (m, 2H), 7.29 (m, 1H), 7.12 (d, J=7.3 Hz, 2H), 6.21 (d, J=16.3 Hz, 1H), 4.88 (d, J=16.3 Hz, 1H), 4.62 (t, J=7.2 Hz, 1H), 2.37 (m, 1H), 2.18 (m, 1H), 0.72 (t, J=7.3 Hz, 3H).

### 3-benzyl-5,6-dibromo-2-(1-1[2-(dimethylamino)ethyl]amino}propyl)thieno[2,3-d]pyrimidin-4(3H)-one (6-7)

A solution of 3-benzyl-5,6-dibromo-2-(1-bromopropyl)thieno[2,3-d]pyrimidin-4(3H)-one (6-5, 35 mg, 0.066 mmol, 1 equiv) and N,N-dimethylethylenediamine (17 mg, 0.198 mmol, 3 equiv) in ethanol (5 mL) was heated at reflux for 18 h. The reaction was concentrated, and the residue was partitioned between EtOAc and brine. The organic layer was dried (MgSO₄) and concentrated to provide 3-benzyl-5,6-dibromo-2-(1-{[2-(dimethylamino)ethyl]amino}propyl)thieno-[2,3-d]pyrimidin-4(3H)-one (6-7) as a yellow gum. MS(M+1)=526.8.

### 3-benzyl-6-bromo-2-(1-{[2-(dimethylamino)ethyl]amino}propyl)thieno[2,3-d]pyrimidin-4(3H)-one (6-8)

A solution of 3-benzyl-6-bromo-2-(1-bromopropyl)thieno[2,3-d]pyrimidin-4(3H)-one (6-6, 35 mg, 0.079 mmol, 1 equiv) and N,N-dimethylethylenediamine (21 mg, 0.237 mmol, 3 equiv) in ethanol (5 mL) was heated at reflux for 18 h. The reaction was concentrated, and the residue was partitioned between EtOAc and brine. The organic layer was dried (MgSO₄) and concentrated to provide 3-benzyl-6-bromo-2-(1-{[2-(dimethylamino)ethyl]amino}-propyl)thieno[2,3-d]pyrimidin-4(3H)-one (6-8) as a yellow gum. MS(M+1)=449.9.

### N-[1-(3-benzyl-5,6-dibromo4-oxo-3,4-dihydrothieno[2,3-d]pyrimidin-2-yl)propyl]-4-bromo-N-[2-(dimethylamino)ethyl]benzamide (6-9)

A solution of 4-bromobenzoyl chloride (19 mg, 0.085 mmol, 1 equiv) in dichloromethane (1 mL) was added to a solution of 3-benzyl-5,6-dibromo-2-(1-{[2-(dimethylamino)ethyl]amino}propyl)thieno[2,3-d]pyrimidin-4(3H)-one (6-8, 45 mg, 0.085 mmol, 1 equiv) and N,N-diisopropylethylamine (11 mg, 0.085 mmol, 1 equiv) in dichloromethane (5 mL), and the resulting reaction mixture was stirred under ambient conditions for 1 h. The reaction mixture was washed with saturated aqueous NaHCO₃ solution, then brine, and dried (MgSO4) and concentrated. The residue was purified by reverse-phase LC (H₂O/CH₃CN gradient w/0.1% TFA present) to provide N-[1-(3-benzyl-5,6-dibromo-4-oxo-3,4-dihydrothieno[2,3-d]pyrimidin-2-yl)propyl]4-bromo-N-[2-(dimethylamino)ethyl]benzamide (6-9) as a colorless foam. MS(M+1)=708.9

### N-[1-(3-benzyl-6-bromo-4-oxo-3,4-dihydrothieno[2,3-d]pyrimidin-2-yl)propyl]-4-bromo-N-[2-(dimethylamino)ethyl]benzamide (6-10)

A solution of 4-bromobenzoyl chloride (19 mg, 0.085 mmol, 1 equiv) in dichloromethane (1 mL) was added to a solution of 3-benzyl-6-bromo-2-(1-{[2-(dimethylamino)ethyl]amino}propyl)thieno[2,3-d]pyrimidin-4(3H)-one (6-9, 38 mg, 0.085 mmol, 1 equiv) and N,N-diisopropylethylamine (11 mg, 0.085 mmol, 1 equiv) in dichloromethane (5 mL), and the resulting reaction mixture was stirred under ambient conditions for 1 h. The reaction mixture was washed with saturated aqueous NaHCO₃ solution, and brine, then dried (MgSO₄) and concentrated. The residue was purified by reverse-phase LC (H₂O/CH₃CN gradient w/0.1% TFA present) to provide N-[1-(3-benzyl-6-bromo-4-oxo-3,4-dihydrothieno[2,3-d]pyrimidin-2-yl)propyl]-4-bromo-N-[2-(dimethylamino)ethyl]benzamide (6-10) as a colorless foam. ¹H NMR (500 MHz, CDCl₃) δ 7.55 (m, 3H), 7.31 (m, 5H), 7.14 (m, 2H), 6.04 (d, J=15.4 Hz, 1H), 5.92 (m, 1H), 5.12 (d, J=15.4 Hz, 1H), 3.37 (m, 2H), 2.05 (m, 4H), 1.83 (m, 6H), 0.65 (m, 3H).

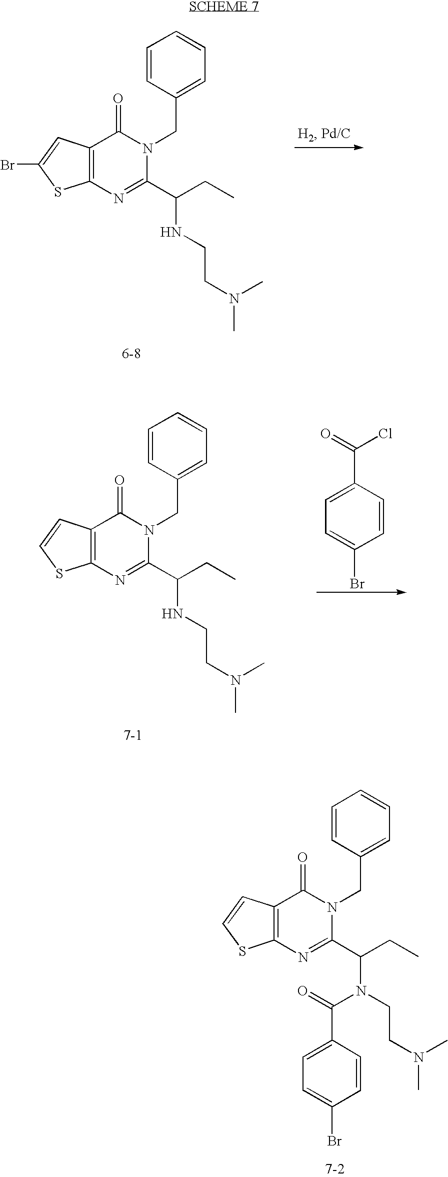

### 3-benzyl-2-(1-{[2-(dimethylamino)ethyl]amino}propyl)thieno[2,3-d]pyrimidin-4(3H)-one (7-1)

A mixture of 3-benzyl-6-bromo-2-(1-{[2-(dimethylamino)ethyl]-amino}propyl)-thieno[2,3-d]pyrimidin-4(3H)-one (6-8, 17 mg, 0.38 mmol, 1 equiv) and 10% Pd/C in ethyl acetate (5 mL) was hydrogenated at 1 atm. for 3 h. The mixture was filtered and the filtrate concentrated to provide 3-benzyl-2-(1-{[2-(dimethylamino)ethyl]amino}propyl)thieno[2,3-d]pyrimidin4(3H)-one (7-1) as a pale yellow gum. MS(M+1)=371.1.

### N-[1-(3-benzyl-4-oxo-3,4-dihydrothieno[2,3-d]pyrimidin-2-yl)propyl]-4-bromo-N-[2-(dimethylamino)ethyl]benzamide (7-2)

A solution of 4-bromobenzoyl chloride (8 mg, 0.035 mmol, 1 equiv) in dichloromethane (1 mL) was added to a solution of 3-benzyl-2-(1-{[2-(dimethylamino)ethyl]amino}propyl)thieno[2,3-d]pyrimidin-4(3H)-one (7-1, 13 mg, 0.035 mmol, 1 equiv) and N,N-diisopropylethylamine (5 mg, 0.035 mmol, 1 equiv) in dichloromethane (1 mL), and the resulting mixture was stirred under ambient conditions for 1 h. The reaction mixture was washed with saturated aqueous NaHCO₃ solution, and brine, then dried (MgSO4) and concentrated. The residue was purified by flash chromatography. Elution with CH₂Cl₂ to 5% NH₃-EtOH/CH₂Cl₂ gave N-[1-(3-benzyl4-oxo-3,4-dihydrothieno[2,3-d]pyrimidin-2-yl)propyl]-4-bromo-N-[2-(dimethylamino)ethyl]benzamide (7-2) as an off-white foam. ¹H NMR (500 MHz, CDCl₃) δ 7.31 (m, 5H), 7.14 (m, 2H), 6.09 (d, J=15.6 Hz, 1H), 5.94 (m, 1H), 5.10 (d, J=15.6 Hz, 1H), 3.40 (m, 2H), 2.11 (m, 1H), 2.03 (m, 2H), 1.87 (m, 1H), 1.79 (s, 6H), 0.66 (t, J=6.6 Hz, 3H).

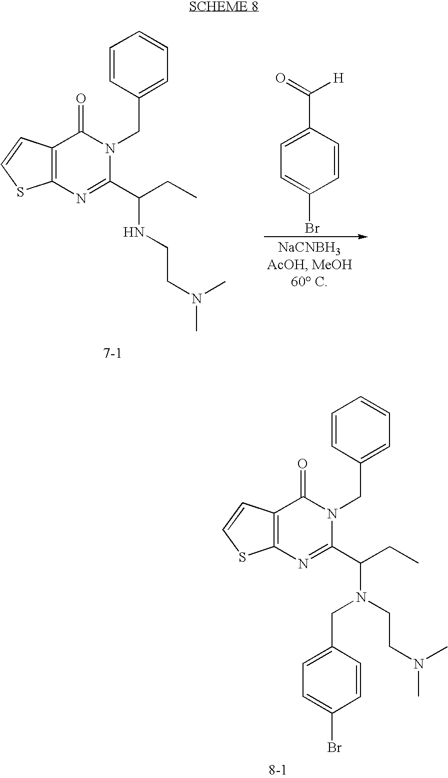

### 3-benzyl-2-(1-{(4-bromobenzyl)[2-(dimethylamino)ethyl]amino}propyl)thieno[2,3-d]pyrimidin-4(3H)-one(8-1)

A solution of 3-benzyl-2-(1-{[2-(dimethylamino)ethyl]amino}-propyl)thieno[2,3-d]pyrimidin-4(3H)-one(7-1, 175 mg, 0.47 mmol, 1 equiv) and 4-bromobenzaldehyde (174 mg, 0.94 mmol, 2 equiv) in methanol (20 mL) was treated with a solution of sodium cyanoborohydride in tetrahydrofuran (1 M, 0.94 mL, 0.94 mmol, 2 equiv). Acetic acid was added to obtain a pH of 6-7 and the reaction was warmed at 60° C. for 18 h. An additional 2 equivalents of 4-bromobenzaldehyde and sodium cyanoborohydride were added after 18, 42 and 66 hours while maintaining the pH at 6-7 with acetic acid. After warming 90 h at 60° C., the reaction was concentrated and the residue was partitioned between EtOAc and aqueous saturated NaHCO₃ solution. The organic layer was washed with brine, dried (MgSO₄) and concentrated. The residue was purified by flash chromatography. Elution with EtOAc to 5% NH₃-EtOH/EtOAC gave 3-benzyl-2-(1-{(4-bromobenzyl)[2-(dimethylamino)ethyl]amino}propyl)thieno[2,3-d]pyrimidin-4(3H)-one(8-1) as a pale yellow gum. ¹H NMR (500 MHz, CDCl₃) δ 7.45 (d, J=6 Hz, 1H), 7.33 (d, J=8 Hz, 2H), 7.21 (m, 4H), 7.05 (d, J=8 Hz, 2H), 6.84 (d, J=7 Hz, 2H), 5.85 (d, J=16 Hz, 1H), 5.32 (d, J=16 Hz, 1H), 3.87 (d, J=14 Hz, 1H), 3.73 (dd, J=11, 3 Hz, 1H), 3.50 (d, J=14 Hz, 1H), 2.92 (m, 1H), 2.61 (m, 1H), 2.28 (m, 2H), 2.15 (m, 1H), 2.07 (s, 6H), 1.74 (m, 1H), 0.64 (t, J=7Hz, 3H).

TABLE 1
REMARK complex 1 with water molecules surrounding it
REMARK r = 0.2114 free_r = 0.2639
REMARK rmsd bonds = 0.006712 rmsd angles = 1.32262
REMARK B rmsd for bonded mainchain atoms = 1.570 target = 1.5
REMARK B rmsd for bonded sidechain atoms = 2.570 targets = 2.0
REMARK B rmsd for angle mainchain atoms = 2.729 target = 2.0
REMARK B rmsd for angle sidechain atoms = 3.936 target = 2.5
REMARK sg = P2(1)2(1)2(1) a = 69.48 b = 79.54 c = 158.98 alpha = 90. beta = 90. gamma = 90.
REMARK reflection file = k2a.cv
REMARK B-correction resolution: 6.0-2.5
REMARK FILENAME = “kin_16dpb.pdb”

| | | | | | | | | | | |
|---|---|---|---|---|---|---|---|---|---|---|
| ATOM | 1 | CB | ASN | 18 | 37.472 | −7.942 | 100.393 | 1.00 | 28.28 | A |
| ATOM | 2 | CG | ASN | 18 | 38.236 | −7.260 | 101.506 | 1.00 | 31.25 | A |
| ATOM | 3 | OD1 | ASN | 18 | 38.752 | −7.913 | 102.413 | 1.00 | 36.19 | A |
| ATOM | 4 | ND2 | ASN | 18 | 38.310 | −5.940 | 101.448 | 1.00 | 32.46 | A |
| ATOM | 5 | C | ASN | 18 | 35.178 | −7.311 | 101.124 | 1.00 | 24.09 | A |
| ATOM | 6 | O | ASN | 18 | 34.900 | −6.997 | 102.284 | 1.00 | 23.76 | A |
| ATOM | 7 | N | ASN | 18 | 35.576 | −9.454 | 99.859 | 1.00 | 25.44 | A |
| ATOM | 8 | CA | ASN | 18 | 36.124 | −8.484 | 100.856 | 1.00 | 25.50 | A |
| ATOM | 9 | N | ILE | 19 | 34.708 | −6.636 | 100.074 | 1.00 | 21.79 | A |
| ATOM | 10 | CA | ILE | 19 | 33.759 | −5.540 | 100.278 | 1.00 | 19.48 | A |
| ATOM | 11 | CB | ILE | 19 | 33.425 | −4.791 | 98.970 | 1.00 | 20.49 | A |
| ATOM | 12 | CG2 | ILE | 19 | 32.124 | −3.992 | 99.129 | 1.00 | 19.87 | A |
| ATOM | 13 | CG1 | ILE | 19 | 34.573 | −3.846 | 98.613 | 1.00 | 20.82 | A |
| ATOM | 14 | CD1 | ILE | 19 | 34.194 | −2.801 | 97.563 | 1.00 | 19.23 | A |
| ATOM | 15 | C | ILE | 19 | 32.487 | −6.185 | 100.820 | 1.00 | 18.08 | A |
| ATOM | 16 | O | ILE | 19 | 31.929 | −7.079 | 100.190 | 1.00 | 17.25 | A |
| ATOM | 17 | N | GLN | 20 | 32.044 | −5.743 | 101.991 | 1.00 | 16.72 | A |
| ATOM | 18 | CA | GLN | 20 | 30.863 | −6.315 | 102.624 | 1.00 | 17.94 | A |
| ATOM | 19 | CB | GLN | 20 | 30.996 | −6.207 | 104.143 | 1.00 | 18.71 | A |
| ATOM | 20 | CG | GLN | 20 | 32.221 | −6.950 | 104.689 | 1.00 | 19.97 | A |
| ATOM | 21 | CD | GLN | 20 | 32.369 | −6.829 | 106.196 | 1.00 | 21.29 | A |
| ATOM | 22 | OE1 | GLN | 20 | 32.511 | −5.730 | 106.734 | 1.00 | 22.63 | A |
| ATOM | 23 | NE2 | GLN | 20 | 32.336 | −7.964 | 106.885 | 1.00 | 22.16 | A |
| ATOM | 24 | C | GLN | 20 | 29.560 | −5.681 | 102.147 | 1.00 | 17.78 | A |
| ATOM | 25 | O | GLN | 20 | 29.396 | −4.462 | 102.184 | 1.00 | 19.12 | A |
| ATOM | 26 | N | VAL | 21 | 28.640 | −6.528 | 101.695 | 1.00 | 14.78 | A |
| ATOM | 27 | CA | VAL | 21 | 27.355 | −6.080 | 101.176 | 1.00 | 13.75 | A |
| ATOM | 28 | CB | VAL | 21 | 27.144 | −6.609 | 99.738 | 1.00 | 14.14 | A |
| ATOM | 29 | CG1 | VAL | 21 | 25.854 | −6.065 | 99.155 | 1.00 | 11.78 | A |
| ATOM | 30 | CG2 | VAL | 21 | 28.339 | −6.238 | 98.875 | 1.00 | 13.09 | A |
| ATOM | 31 | C | VAL | 21 | 26.198 | −6.571 | 102.036 | 1.00 | 14.04 | A |
| ATOM | 32 | O | VAL | 21 | 26.128 | −7.756 | 102.365 | 1.00 | 13.35 | A |
| ATOM | 33 | N | VAL | 22 | 25.294 | −5.659 | 102.396 | 1.00 | 14.49 | A |
| ATOM | 34 | CA | VAL | 22 | 24.123 | −6.011 | 103.194 | 1.00 | 14.01 | A |
| ATOM | 35 | CB | VAL | 22 | 24.197 | −5.423 | 104.627 | 1.00 | 15.50 | A |
| ATOM | 36 | CG1 | VAL | 22 | 25.588 | −5.628 | 105.201 | 1.00 | 16.80 | A |
| ATOM | 37 | CG2 | VAL | 22 | 23.817 | −3.968 | 104.623 | 1.00 | 15.97 | A |
| ATOM | 38 | C | VAL | 22 | 22.838 | −5.518 | 102.532 | 1.00 | 13.29 | A |
| ATOM | 39 | O | VAL | 22 | 22.811 | −4.469 | 101.884 | 1.00 | 13.40 | A |
| ATOM | 40 | N | VAL | 23 | 21.773 | −6.292 | 102.694 | 1.00 | 12.04 | A |
| ATOM | 41 | CA | VAL | 23 | 20.478 | −5.953 | 102.125 | 1.00 | 11.16 | A |
| ATOM | 42 | CB | VAL | 23 | 19.890 | −7.155 | 101.350 | 1.00 | 10.39 | A |
| ATOM | 43 | CG1 | VAL | 23 | 18.423 | −6.883 | 100.979 | 1.00 | 6.97 | A |
| ATOM | 44 | CG2 | VAL | 23 | 20.733 | −7.429 | 100.112 | 1.00 | 5.75 | A |
| ATOM | 45 | C | VAL | 23 | 19.496 | −5.551 | 103.220 | 1.00 | 12.26 | A |
| ATOM | 46 | O | VAL | 23 | 19.433 | −6.180 | 104.276 | 1.00 | 12.72 | A |
| ATOM | 47 | N | ARG | 24 | 18.734 | −4.497 | 102.965 | 1.00 | 12.29 | A |
| ATOM | 48 | CA | ARG | 24 | 17.741 | −4.033 | 103.925 | 1.00 | 11.98 | A |
| ATOM | 49 | CB | ARG | 24 | 18.150 | −2.711 | 104.572 | 1.00 | 9.94 | A |
| ATOM | 50 | CG | ARG | 24 | 17.092 | −2.197 | 105.533 | 1.00 | 9.40 | A |
| ATOM | 51 | CD | ARG | 24 | 17.412 | −0.826 | 106.110 | 1.00 | 11.24 | A |
| ATOM | 52 | NE | ARG | 24 | 16.638 | −0.585 | 107.326 | 1.00 | 8.87 | A |
| ATOM | 53 | CZ | ARG | 24 | 16.668 | 0.540 | 108.033 | 1.00 | 11.40 | A |
| ATOM | 54 | NH1 | ARG | 24 | 17.432 | 1.563 | 107.649 | 1.00 | 11.52 | A |
| ATOM | 55 | NH2 | ARG | 24 | 15.956 | 0.629 | 109.151 | 1.00 | 12.63 | A |
| ATOM | 56 | C | ARG | 24 | 16.404 | −3.831 | 103.230 | 1.00 | 13.62 | A |
| ATOM | 57 | O | ARG | 24 | 16.248 | −2.918 | 102.415 | 1.00 | 14.61 | A |
| ATOM | 58 | N | CYS | 25 | 15.446 | −4.690 | 103.553 | 1.00 | 12.77 | A |
| ATOM | 59 | CA | CYS | 25 | 14.117 | −4.599 | 102.983 | 1.00 | 13.88 | A |
| ATOM | 60 | CB | CYS | 25 | 13.461 | −5.980 | 102.951 | 1.00 | 15.60 | A |
| ATOM | 61 | SG | CYS | 25 | 11.855 | −6.006 | 102.134 | 1.00 | 21.58 | A |
| ATOM | 62 | C | CYS | 25 | 13.292 | −3.675 | 103.865 | 1.00 | 13.78 | A |
| ATOM | 63 | O | CYS | 25 | 13.293 | −3.820 | 105.084 | 1.00 | 15.62 | A |
| ATOM | 64 | N | ARG | 26 | 12.605 | −2.713 | 103.261 | 1.00 | 12.12 | A |
| ATOM | 65 | CA | ARG | 26 | 11.774 | −1.815 | 104.045 | 1.00 | 12.61 | A |
| ATOM | 66 | CB | ARG | 26 | 11.601 | −0.465 | 103.343 | 1.00 | 10.76 | A |
| ATOM | 67 | CG | ARG | 26 | 10.679 | −0.499 | 102.128 | 1.00 | 7.66 | A |
| ATOM | 68 | CD | ARG | 26 | 10.181 | 0.890 | 101.775 | 1.00 | 7.16 | A |
| ATOM | 69 | NE | ARG | 26 | 9.592 | 0.934 | 100.442 | 1.00 | 7.55 | A |
| ATOM | 70 | CZ | ARG | 26 | 8.413 | 0.411 | 100.125 | 1.00 | 8.80 | A |
| ATOM | 71 | NH1 | ARG | 26 | 7.677 | −0.194 | 101.052 | 1.00 | 8.81 | A |
| ATOM | 72 | NH2 | ARG | 26 | 7.980 | 0.472 | 98.876 | 1.00 | 7.02 | A |
| ATOM | 73 | C | ARG | 26 | 10.407 | −2.470 | 104.215 | 1.00 | 15.65 | A |
| ATOM | 74 | O | ARG | 26 | 10.058 | −3.420 | 103.500 | 1.00 | 17.10 | A |
| ATOM | 75 | N | PRO | 27 | 9.615 | −1.982 | 105.170 | 1.00 | 17.31 | A |
| ATOM | 76 | CD | PRO | 27 | 9.957 | −1.053 | 106.262 | 1.00 | 18.01 | A |
| ATOM | 77 | CA | PRO | 27 | 8.287 | −2.562 | 105.382 | 1.00 | 20.54 | A |
| ATOM | 78 | CB | PRO | 27 | 8.037 | −2.277 | 106.858 | 1.00 | 19.92 | A |
| ATOM | 79 | CG | PRO | 27 | 8.639 | −0.932 | 107.017 | 1.00 | 17.88 | A |
| ATOM | 80 | C | PRO | 27 | 7.237 | −1.897 | 104.492 | 1.00 | 23.41 | A |
| ATOM | 81 | O | PRO | 27 | 7.482 | −0.833 | 103.916 | 1.00 | 23.28 | A |
| ATOM | 82 | N | PHE | 28 | 6.080 | −2.542 | 104.371 | 1.00 | 26.52 | A |
| ATOM | 83 | CA | PHE | 28 | 4.976 | −2.003 | 103.584 | 1.00 | 29.18 | A |
| ATOM | 84 | CB | PHE | 28 | 3.805 | −2.982 | 103.588 | 1.00 | 27.65 | A |
| ATOM | 85 | CG | PHE | 28 | 3.948 | −4.107 | 102.610 | 1.00 | 28.35 | A |
| ATOM | 86 | CD1 | PHE | 28 | 3.947 | −5.425 | 103.045 | 1.00 | 28.03 | A |
| ATOM | 87 | CD2 | PHE | 28 | 4.038 | −3.850 | 101.243 | 1.00 | 27.68 | A |
| ATOM | 88 | CE1 | PHE | 28 | 4.026 | −6.477 | 102.139 | 1.00 | 27.56 | A |
| ATOM | 89 | CE2 | PHE | 28 | 4.119 | −4.893 | 100.324 | 1.00 | 29.26 | A |
| ATOM | 90 | CZ | PHE | 28 | 4.112 | −6.212 | 100.773 | 1.00 | 27.81 | A |
| ATOM | 91 | C | PHE | 28 | 4.513 | −0.680 | 104.191 | 1.00 | 32.56 | A |
| ATOM | 92 | O | PHE | 28 | 4.426 | −0.548 | 105.411 | 1.00 | 33.43 | A |
| ATOM | 93 | N | ASN | 29 | 4.217 | 0.299 | 103.345 | 1.00 | 37.21 | A |
| ATOM | 94 | CA | ASN | 29 | 3.744 | 1.595 | 103.829 | 1.00 | 42.32 | A |
| ATOM | 95 | CB | ASN | 29 | 4.073 | 2.692 | 102.809 | 1.00 | 42.04 | A |
| ATOM | 96 | CG | ASN | 29 | 3.604 | 2.344 | 101.410 | 1.00 | 41.31 | A |
| ATOM | 97 | OD1 | ASN | 29 | 2.409 | 2.177 | 101.168 | 1.00 | 41.82 | A |
| ATOM | 98 | ND2 | ASN | 29 | 4.546 | 2.228 | 100.482 | 1.00 | 40.11 | A |
| ATOM | 99 | C | ASN | 29 | 2.232 | 1.526 | 104.054 | 1.00 | 46.51 | A |
| ATOM | 100 | O | ASN | 29 | 1.606 | 0.505 | 103.768 | 1.00 | 46.59 | A |
| ATOM | 101 | N | LEU | 30 | 1.650 | 2.612 | 104.562 | 1.00 | 51.19 | A |
| ATOM | 102 | CA | LEU | 30 | 0.212 | 2.661 | 104.826 | 1.00 | 54.81 | A |
| ATOM | 103 | CB | LEU | 30 | −0.178 | 4.040 | 105.362 | 1.00 | 56.40 | A |
| ATOM | 104 | CG | LEU | 30 | −1.659 | 4.234 | 105.705 | 1.00 | 58.19 | A |
| ATOM | 105 | CD1 | LEU | 30 | −2.058 | 3.273 | 106.820 | 1.00 | 57.83 | A |
| ATOM | 106 | CD2 | LEU | 30 | −1.899 | 5.680 | 106.130 | 1.00 | 59.11 | A |
| ATOM | 107 | C | LEU | 30 | −0.637 | 2.343 | 103.592 | 1.00 | 56.70 | A |
| ATOM | 108 | O | LEU | 30 | −1.552 | 1.525 | 103.658 | 1.00 | 56.66 | A |
| ATOM | 109 | N | ALA | 31 | −0.329 | 2.992 | 102.471 | 1.00 | 59.03 | A |
| ATOM | 110 | CA | ALA | 31 | −1.062 | 2.787 | 101.222 | 1.00 | 61.19 | A |
| ATOM | 111 | CB | ALA | 31 | −0.414 | 3.591 | 100.100 | 1.00 | 61.28 | A |
| ATOM | 112 | C | ALA | 31 | −1.125 | 1.316 | 100.833 | 1.00 | 62.78 | A |
| ATOM | 113 | O | ALA | 31 | −2.123 | 0.850 | 100.282 | 1.00 | 62.16 | A |
| ATOM | 114 | N | GLU | 32 | −0.048 | 0.593 | 101.117 | 1.00 | 65.22 | A |
| ATOM | 115 | CA | GLU | 32 | 0.031 | −0.827 | 100.801 | 1.00 | 67.27 | A |
| ATOM | 116 | CB | GLU | 32 | 1.501 | −1.249 | 100.702 | 1.00 | 66.96 | A |
| ATOM | 117 | CG | GLU | 32 | 2.199 | −0.712 | 99.453 | 1.00 | 67.12 | A |
| ATOM | 118 | CD | GLU | 32 | 3.713 | −0.641 | 99.590 | 1.00 | 67.26 | A |
| ATOM | 119 | OE1 | GLU | 32 | 4.392 | −0.422 | 98.563 | 1.00 | 66.83 | A |
| ATOM | 120 | OE2 | GLU | 32 | 4.223 | −0.792 | 100.723 | 1.00 | 65.99 | A |
| ATOM | 121 | C | GLU | 32 | −0.706 | −1.666 | 101.844 | 1.00 | 68.26 | A |
| ATOM | 122 | O | GLU | 32 | −1.260 | −2.716 | 101.526 | 1.00 | 68.16 | A |
| ATOM | 123 | N | ARG | 33 | −0.722 | −1.191 | 103.087 | 1.00 | 69.65 | A |
| ATOM | 124 | CA | ARG | 33 | −1.403 | −1.897 | 104.169 | 1.00 | 71.22 | A |
| ATOM | 125 | CB | ARG | 33 | −1.196 | −1.162 | 105.498 | 1.00 | 72.33 | A |
| ATOM | 126 | CG | ARG | 33 | 0.239 | −1.138 | 106.009 | 1.00 | 73.65 | A |
| ATOM | 127 | CD | ARG | 33 | 0.695 | −2.508 | 106.479 | 1.00 | 74.57 | A |
| ATOM | 128 | NE | ARG | 33 | 2.043 | −2.462 | 107.041 | 1.00 | 76.44 | A |
| ATOM | 129 | CZ | ARG | 33 | 2.692 | −3.517 | 107.521 | 1.00 | 76.91 | A |
| ATOM | 130 | NH1 | ARG | 33 | 2.119 | −4.714 | 107.513 | 1.00 | 76.68 | A |
| ATOM | 131 | NH2 | ARG | 33 | 3.918 | −3.376 | 108.007 | 1.00 | 77.35 | A |
| ATOM | 132 | C | ARG | 33 | −2.901 | −2.013 | 103.885 | 1.00 | 71.74 | A |
| ATOM | 133 | O | ARG | 33 | −3.464 | −3.111 | 103.900 | 1.00 | 71.46 | A |
| ATOM | 134 | N | LYS | 34 | −3.536 | −0.870 | 103.632 | 1.00 | 71.80 | A |
| ATOM | 135 | CA | LYS | 34 | −4.967 | −0.817 | 103.349 | 1.00 | 71.67 | A |
| ATOM | 136 | CB | LYS | 34 | −5.426 | 0.641 | 103.195 | 1.00 | 72.94 | A |
| ATOM | 137 | CG | LYS | 34 | −4.734 | 1.407 | 102.072 | 1.00 | 74.72 | A |
| ATOM | 138 | CD | LYS | 34 | −5.218 | 2.856 | 101.986 | 1.00 | 75.69 | A |
| ATOM | 139 | CE | LYS | 34 | −6.680 | 2.936 | 101.565 | 1.00 | 75.79 | A |
| ATOM | 140 | NZ | LYS | 34 | −7.149 | 4.343 | 101.426 | 1.00 | 74.45 | A |
| ATOM | 141 | C | LYS | 34 | −5.315 | −1.604 | 102.088 | 1.00 | 70.68 | A |
| ATOM | 142 | O | LYS | 34 | −6.448 | −2.064 | 101.924 | 1.00 | 70.80 | A |
| ATOM | 143 | N | ALA | 35 | −4.338 | −1.753 | 101.198 | 1.00 | 68.59 | A |
| ATOM | 144 | CA | ALA | 35 | −4.539 | −2.501 | 99.963 | 1.00 | 66.37 | A |
| ATOM | 145 | CB | ALA | 35 | −3.639 | −1.949 | 98.861 | 1.00 | 65.65 | A |
| ATOM | 146 | C | ALA | 35 | −4.199 | −3.960 | 100.241 | 1.00 | 64.89 | A |
| ATOM | 147 | O | ALA | 35 | −4.277 | −4.807 | 99.352 | 1.00 | 64.01 | A |
| ATOM | 148 | N | SER | 36 | −3.825 | −4.233 | 101.491 | 1.00 | 63.72 | A |
| ATOM | 149 | CA | SER | 36 | −3.454 | −5.574 | 101.937 | 1.00 | 62.31 | A |
| ATOM | 150 | CB | SER | 36 | −4.711 | −6.422 | 102.194 | 1.00 | 62.73 | A |
| ATOM | 151 | OG | SER | 36 | −5.556 | −6.469 | 101.056 | 1.00 | 63.14 | A |
| ATOM | 152 | C | SER | 36 | −2.542 | −6.261 | 100.920 | 1.00 | 60.52 | A |
| ATOM | 153 | O | SER | 36 | −2.933 | −7.223 | 100.256 | 1.00 | 60.52 | A |
| ATOM | 154 | N | ALA | 37 | −1.316 | −5.759 | 100.818 | 1.00 | 57.81 | A |
| ATOM | 155 | CA | ALA | 37 | −0.339 | −6.291 | 99.877 | 1.00 | 54.58 | A |
| ATOM | 156 | CB | ALA | 37 | 0.709 | −5.228 | 99.561 | 1.00 | 53.39 | A |
| ATOM | 157 | C | ALA | 37 | 0.351 | −7.562 | 100.359 | 1.00 | 51.84 | A |
| ATOM | 158 | O | ALA | 37 | 0.586 | −7.754 | 101.554 | 1.00 | 50.84 | A |
| ATOM | 159 | N | HIS | 38 | 0.669 | −8.429 | 99.405 | 1.00 | 48.60 | A |
| ATOM | 160 | CA | HIS | 38 | 1.363 | −9.672 | 99.690 | 1.00 | 45.12 | A |
| ATOM | 161 | CB | HIS | 38 | 0.775 | −10.810 | 98.840 | 1.00 | 48.05 | A |
| ATOM | 162 | CG | HIS | 38 | 0.753 | −10.528 | 97.364 | 1.00 | 50.18 | A |
| ATOM | 163 | CD2 | HIS | 38 | −0.262 | −10.171 | 96.542 | 1.00 | 51.32 | A |
| ATOM | 164 | ND1 | HIS | 38 | 1.875 | −10.621 | 96.566 | 1.00 | 50.53 | A |
| ATOM | 165 | CE1 | HIS | 38 | 1.552 | −10.337 | 95.317 | 1.00 | 50.82 | A |
| ATOM | 166 | NE2 | HIS | 38 | 0.261 | −10.059 | 95.275 | 1.00 | 51.95 | A |
| ATOM | 167 | C | HIS | 38 | 2.836 | −9.436 | 99.350 | 1.00 | 40.69 | A |
| ATOM | 168 | O | HIS | 38 | 3.165 | −9.005 | 98.244 | 1.00 | 39.51 | A |
| ATOM | 169 | N | SER | 39 | 3.714 | −9.692 | 100.312 | 1.00 | 34.50 | A |
| ATOM | 170 | CA | SER | 39 | 5.138 | −9.494 | 100.106 | 1.00 | 29.81 | A |
| ATOM | 171 | CB | SER | 39 | 5.860 | −9.458 | 101.449 | 1.00 | 29.59 | A |
| ATOM | 172 | OG | SER | 39 | 7.263 | −9.361 | 101.265 | 1.00 | 30.93 | A |
| ATOM | 173 | C | SER | 39 | 5.753 | −10.578 | 99.242 | 1.00 | 27.18 | A |
| ATOM | 174 | O | SER | 39 | 5.507 | −11.758 | 99.456 | 1.00 | 27.84 | A |
| ATOM | 175 | N | ILE | 40 | 6.556 | −10.179 | 98.263 | 1.00 | 23.70 | A |
| ATOM | 176 | CA | ILE | 40 | 7.216 | −11.148 | 97.403 | 1.00 | 20.93 | A |
| ATOM | 177 | CB | ILE | 40 | 7.278 | −10.677 | 95.945 | 1.00 | 21.59 | A |
| ATOM | 178 | CG2 | ILE | 40 | 5.868 | −10.554 | 95.381 | 1.00 | 21.07 | A |
| ATOM | 179 | CG1 | ILE | 40 | 8.025 | −9.343 | 95.857 | 1.00 | 21.01 | A |
| ATOM | 180 | CD1 | ILE | 40 | 8.377 | −8.954 | 94.443 | 1.00 | 17.86 | A |
| ATOM | 181 | C | ILE | 40 | 8.638 | −11.366 | 97.895 | 1.00 | 19.29 | A |
| ATOM | 182 | O | ILE | 40 | 9.395 | −12.130 | 97.306 | 1.00 | 18.82 | A |
| ATOM | 183 | N | VAL | 41 | 8.987 | −10.696 | 98.988 | 1.00 | 18.43 | A |
| ATOM | 184 | CA | VAL | 41 | 10.316 | −10.801 | 99.572 | 1.00 | 19.01 | A |
| ATOM | 185 | CB | VAL | 41 | 10.974 | −9.394 | 99.666 | 1.00 | 18.10 | A |
| ATOM | 186 | CG1 | VAL | 41 | 12.231 | −9.448 | 100.525 | 1.00 | 17.03 | A |
| ATOM | 187 | CG2 | VAL | 41 | 11.303 | −8.881 | 98.279 | 1.00 | 16.81 | A |
| ATOM | 188 | C | VAL | 41 | 10.286 | −11.420 | 100.976 | 1.00 | 21.10 | A |
| ATOM | 189 | O | VAL | 41 | 9.401 | −11.122 | 101.779 | 1.00 | 22.16 | A |
| ATOM | 190 | N | GLU | 42 | 11.247 | −12.286 | 101.269 | 1.00 | 21.96 | A |
| ATOM | 191 | CA | GLU | 42 | 11.336 | −12.894 | 102.595 | 1.00 | 24.43 | A |
| ATOM | 192 | CB | GLU | 42 | 10.758 | −14.310 | 102.588 | 1.00 | 26.41 | A |
| ATOM | 193 | CG | GLU | 42 | 9.235 | −14.321 | 102.535 | 1.00 | 33.53 | A |
| ATOM | 194 | CD | GLU | 42 | 8.646 | −15.717 | 102.435 | 1.00 | 37.53 | A |
| ATOM | 195 | OE1 | GLU | 42 | 7.400 | −15.830 | 102.388 | 1.00 | 37.91 | A |
| ATOM | 196 | OE2 | GLU | 42 | 9.425 | −16.695 | 102.399 | 1.00 | 39.48 | A |
| ATOM | 197 | C | GLU | 42 | 12.790 | −12.912 | 103.042 | 1.00 | 23.06 | A |
| ATOM | 198 | O | GLU | 42 | 13.672 | −13.313 | 102.284 | 1.00 | 23.11 | A |
| ATOM | 199 | N | CYS | 43 | 13.040 | −12.461 | 104.267 | 1.00 | 22.56 | A |
| ATOM | 200 | CA | CYS | 43 | 14.399 | −12.417 | 104.792 | 1.00 | 22.27 | A |
| ATOM | 201 | CB | CYS | 43 | 14.688 | −11.032 | 105.350 | 1.00 | 21.27 | A |
| ATOM | 202 | SG | CYS | 43 | 14.515 | −9.727 | 104.119 | 1.00 | 26.40 | A |
| ATOM | 203 | C | CYS | 43 | 14.683 | −13.458 | 105.861 | 1.00 | 23.32 | A |
| ATOM | 204 | O | CYS | 43 | 13.795 | −13.850 | 106.617 | 1.00 | 25.24 | A |
| ATOM | 205 | N | ASP | 44 | 15.936 | −13.900 | 105.909 | 1.00 | 24.35 | A |
| ATOM | 206 | CA | ASP | 44 | 16.398 | −14.897 | 106.873 | 1.00 | 24.49 | A |
| ATOM | 207 | CB | ASP | 44 | 16.579 | −16.251 | 106.182 | 1.00 | 24.72 | A |
| ATOM | 208 | CG | ASP | 44 | 16.638 | −17.408 | 107.164 | 1.00 | 27.03 | A |
| ATOM | 209 | OD1 | ASP | 44 | 17.089 | −17.201 | 108.313 | 1.00 | 28.16 | A |
| ATOM | 210 | OD2 | ASP | 44 | 16.249 | −18.531 | 106.780 | 1.00 | 27.08 | A |
| ATOM | 211 | C | ASP | 44 | 17.745 | −14.403 | 107.404 | 1.00 | 24.36 | A |
| ATOM | 212 | O | ASP | 44 | 18.804 | −14.795 | 106.923 | 1.00 | 23.06 | A |
| ATOM | 213 | N | PRO | 45 | 17.721 | −13.527 | 108.411 | 1.00 | 25.65 | A |
| ATOM | 214 | CD | PRO | 45 | 16.551 | −12.911 | 109.059 | 1.00 | 25.98 | A |
| ATOM | 215 | CA | PRO | 45 | 18.967 | −12.999 | 108.971 | 1.00 | 26.11 | A |
| ATOM | 216 | CB | PRO | 45 | 18.482 | −12.143 | 110.133 | 1.00 | 25.67 | A |
| ATOM | 217 | CG | PRO | 45 | 17.153 | −11.658 | 109.657 | 1.00 | 26.57 | A |
| ATOM | 218 | C | PRO | 45 | 19.972 | −14.051 | 109.418 | 1.00 | 26.95 | A |
| ATOM | 219 | O | PRO | 45 | 21.159 | −13.952 | 109.111 | 1.00 | 26.64 | A |
| ATOM | 220 | N | VAL | 46 | 19.502 | −15.059 | 110.140 | 1.00 | 27.42 | A |
| ATOM | 221 | CA | VAL | 46 | 20.401 | −16.088 | 110.636 | 1.00 | 28.91 | A |
| ATOM | 222 | CB | VAL | 46 | 19.634 | −17.105 | 111.522 | 1.00 | 28.55 | A |
| ATOM | 223 | CG1 | VAL | 46 | 18.882 | −18.096 | 110.655 | 1.00 | 28.05 | A |
| ATOM | 224 | CG2 | VAL | 46 | 20.600 | −17.807 | 112.465 | 1.00 | 28.65 | A |
| ATOM | 225 | C | VAL | 46 | 21.148 | −16.810 | 109.506 | 1.00 | 30.17 | A |
| ATOM | 226 | O | VAL | 46 | 22.279 | −17.264 | 109.688 | 1.00 | 29.93 | A |
| ATOM | 227 | N | ARG | 47 | 20.530 | −16.893 | 108.333 | 1.00 | 30.73 | A |
| ATOM | 228 | CA | ARG | 47 | 21.161 | −17.552 | 107.195 | 1.00 | 31.90 | A |
| ATOM | 229 | CB | ARG | 47 | 20.156 | −18.495 | 106.515 | 1.00 | 35.93 | A |
| ATOM | 230 | CG | ARG | 47 | 19.909 | −19.796 | 107.286 | 1.00 | 43.15 | A |
| ATOM | 231 | CD | ARG | 47 | 18.670 | −20.554 | 106.799 | 1.00 | 48.31 | A |
| ATOM | 232 | NE | ARG | 47 | 18.660 | −20.769 | 105.352 | 1.00 | 52.94 | A |
| ATOM | 233 | CZ | ARG | 47 | 17.705 | −21.426 | 104.697 | 1.00 | 53.97 | A |
| ATOM | 234 | NH1 | ARG | 47 | 16.675 | −21.940 | 105.356 | 1.00 | 54.33 | A |
| ATOM | 235 | NH2 | ARG | 47 | 17.773 | −21.561 | 103.381 | 1.00 | 54.58 | A |
| ATOM | 236 | C | ARG | 47 | 21.736 | −16.560 | 106.171 | 1.00 | 30.25 | A |
| ATOM | 237 | O | ARG | 47 | 22.232 | −16.965 | 105.122 | 1.00 | 27.99 | A |
| ATOM | 238 | N | LYS | 48 | 21.682 | −15.266 | 106.484 | 1.00 | 29.50 | A |
| ATOM | 239 | CA | LYS | 48 | 22.200 | −14.228 | 105.586 | 1.00 | 28.39 | A |
| ATOM | 240 | CB | LYS | 48 | 23.719 | −14.362 | 105.425 | 1.00 | 28.24 | A |
| ATOM | 241 | CG | LYS | 48 | 24.497 | −14.762 | 106.662 | 1.00 | 29.13 | A |
| ATOM | 242 | CD | LYS | 48 | 24.560 | −13.656 | 107.677 | 1.00 | 31.53 | A |
| ATOM | 243 | CE | LYS | 48 | 25.701 | −13.897 | 108.651 | 1.00 | 34.18 | A |
| ATOM | 244 | NZ | LYS | 48 | 27.015 | −13.908 | 107.950 | 1.00 | 34.16 | A |
| ATOM | 245 | C | LYS | 48 | 21.564 | −14.415 | 104.209 | 1.00 | 27.13 | A |
| ATOM | 246 | O | LYS | 48 | 22.244 | −14.330 | 103.188 | 1.00 | 27.94 | A |
| ATOM | 247 | N | GLU | 49 | 20.261 | −14.645 | 104.170 | 1.00 | 25.69 | A |
| ATOM | 248 | CA | GLU | 49 | 19.616 | −14.908 | 102.895 | 1.00 | 26.19 | A |
| ATOM | 249 | CB | GLU | 49 | 19.300 | −16.398 | 102.827 | 1.00 | 28.94 | A |
| ATOM | 250 | CG | GLU | 49 | 18.711 | −16.897 | 101.534 | 1.00 | 34.48 | A |
| ATOM | 251 | CD | GLU | 49 | 18.082 | −18.269 | 101.710 | 1.00 | 39.36 | A |
| ATOM | 252 | OE1 | GLU | 49 | 16.880 | −18.326 | 102.067 | 1.00 | 40.10 | A |
| ATOM | 253 | OE2 | GLU | 49 | 18.794 | −19.285 | 101.516 | 1.00 | 39.93 | A |
| ATOM | 254 | C | GLU | 49 | 18.355 | −14.113 | 102.607 | 1.00 | 24.38 | A |
| ATOM | 255 | O | GLU | 49 | 17.545 | −13.868 | 103.496 | 1.00 | 24.72 | A |
| ATOM | 256 | N | VAL | 50 | 18.196 | −13.715 | 101.349 | 1.00 | 22.04 | A |
| ATOM | 257 | CA | VAL | 50 | 17.010 | −12.989 | 100.928 | 1.00 | 21.18 | A |
| ATOM | 258 | CB | VAL | 50 | 17.350 | −11.553 | 100.410 | 1.00 | 21.63 | A |
| ATOM | 259 | CG1 | VAL | 50 | 18.150 | −11.619 | 99.127 | 1.00 | 21.68 | A |
| ATOM | 260 | CG2 | VAL | 50 | 16.071 | −10.764 | 100.190 | 1.00 | 21.12 | A |
| ATOM | 261 | C | VAL | 50 | 16.392 | −13.834 | 99.821 | 1.00 | 19.98 | A |
| ATOM | 262 | O | VAL | 50 | 17.088 | −14.282 | 98.912 | 1.00 | 20.15 | A |
| ATOM | 263 | N | SER | 51 | 15.087 | −14.074 | 99.917 | 1.00 | 21.09 | A |
| ATOM | 264 | CA | SER | 51 | 14.368 | −14.890 | 98.934 | 1.00 | 21.32 | A |
| ATOM | 265 | CB | SER | 51 | 13.742 | −16.106 | 99.629 | 1.00 | 20.35 | A |
| ATOM | 266 | OG | SER | 51 | 13.065 | −16.943 | 98.712 | 1.00 | 23.49 | A |
| ATOM | 267 | C | SER | 51 | 13.280 | −14.067 | 98.256 | 1.00 | 20.53 | A |
| ATOM | 268 | O | SER | 51 | 12.496 | −13.401 | 98.925 | 1.00 | 21.64 | A |
| ATOM | 269 | N | VAL | 52 | 13.237 | −14.107 | 96.929 | 1.00 | 21.28 | A |
| ATOM | 270 | CA | VAL | 52 | 12.238 | −13.348 | 96.189 | 1.00 | 22.46 | A |
| ATOM | 271 | CB | VAL | 52 | 12.892 | −12.293 | 95.282 | 1.00 | 21.66 | A |
| ATOM | 272 | CG1 | VAL | 52 | 11.813 | −11.462 | 94.605 | 1.00 | 18.69 | A |
| ATOM | 273 | CG2 | VAL | 52 | 13.835 | −11.417 | 96.091 | 1.00 | 19.80 | A |
| ATOM | 274 | C | VAL | 52 | 11.336 | −14.220 | 95.322 | 1.00 | 24.82 | A |
| ATOM | 275 | O | VAL | 52 | 11.802 | −15.099 | 94.597 | 1.00 | 26.25 | A |
| ATOM | 276 | N | ARG | 53 | 10.036 | −13.964 | 95.409 | 1.00 | 27.28 | A |
| ATOM | 277 | CA | ARG | 53 | 9.034 | −14.690 | 94.638 | 1.00 | 29.70 | A |
| ATOM | 278 | CB | ARG | 53 | 7.679 | −14.562 | 95.341 | 1.00 | 29.44 | A |
| ATOM | 279 | CG | ARG | 53 | 6.511 | −15.238 | 94.658 | 1.00 | 32.62 | A |
| ATOM | 280 | CD | ARG | 53 | 5.277 | −15.124 | 95.536 | 1.00 | 32.33 | A |
| ATOM | 281 | NE | ARG | 53 | 5.486 | −15.812 | 96.805 | 1.00 | 34.30 | A |
| ATOM | 282 | CZ | ARG | 53 | 4.754 | −15.618 | 97.894 | 1.00 | 35.73 | A |
| ATOM | 283 | NH1 | ARG | 53 | 3.751 | −14.743 | 97.877 | 1.00 | 35.26 | A |
| ATOM | 284 | NH2 | ARG | 53 | 5.029 | −16.297 | 99.001 | 1.00 | 33.24 | A |
| ATOM | 285 | C | ARG | 53 | 8.992 | −14.062 | 93.243 | 1.00 | 30.22 | A |
| ATOM | 286 | O | ARG | 53 | 8.554 | −12.922 | 93.080 | 1.00 | 28.70 | A |
| ATOM | 287 | N | THR | 54 | 9.457 | −14.809 | 92.244 | 1.00 | 32.13 | A |
| ATOM | 288 | CA | THR | 54 | 9.506 | −14.314 | 90.872 | 1.00 | 35.09 | A |
| ATOM | 289 | CB | THR | 54 | 10.785 | −14.788 | 90.153 | 1.00 | 34.03 | A |
| ATOM | 290 | OG1 | THR | 54 | 10.798 | −16.218 | 90.086 | 1.00 | 33.22 | A |
| ATOM | 291 | OG2 | THR | 54 | 12.026 | −14.305 | 90.898 | 1.00 | 33.36 | A |
| ATOM | 292 | C | THR | 54 | 8.317 | −14.705 | 90.011 | 1.00 | 38.38 | A |
| ATOM | 293 | O | THR | 54 | 8.081 | −14.098 | 88.970 | 1.00 | 39.08 | A |
| ATOM | 294 | N | GLY | 55 | 7.574 | −15.717 | 90.435 | 1.00 | 42.35 | A |
| ATOM | 295 | CA | GLY | 55 | 6.433 | −16.145 | 89.653 | 1.00 | 47.68 | A |
| ATOM | 296 | C | GLY | 55 | 5.137 | −15.562 | 90.171 | 1.00 | 52.55 | A |
| ATOM | 297 | O | GLY | 55 | 4.638 | −14.562 | 89.651 | 1.00 | 52.62 | A |
| ATOM | 298 | N | GLY | 56 | 4.589 | −16.196 | 91.204 | 1.00 | 56.07 | A |
| ATOM | 299 | CA | GLY | 56 | 3.343 | −15.734 | 91.789 | 1.00 | 58.64 | A |
| ATOM | 300 | C | GLY | 56 | 2.660 | −16.804 | 92.620 | 1.00 | 60.65 | A |
| ATOM | 301 | O | GLY | 56 | 2.917 | −17.999 | 92.444 | 1.00 | 60.57 | A |
| ATOM | 302 | N | LEU | 57 | 1.795 | −16.364 | 93.532 | 1.00 | 62.43 | A |
| ATOM | 303 | CA | LEU | 57 | 1.039 | −17.253 | 94.421 | 1.00 | 63.41 | A |
| ATOM | 304 | CB | LEU | 57 | 0.439 | −18.425 | 93.627 | 1.00 | 63.91 | A |
| ATOM | 305 | CG | LEU | 57 | −0.466 | −18.152 | 92.419 | 1.00 | 64.67 | A |
| ATOM | 306 | CD1 | LEU | 57 | −0.951 | −19.486 | 91.873 | 1.00 | 64.88 | A |
| ATOM | 307 | CD2 | LEU | 57 | −1.654 | −17.276 | 92.806 | 1.00 | 64.92 | A |
| ATOM | 308 | C | LEU | 57 | 1.873 | −17.800 | 95.586 | 1.00 | 63.25 | A |
| ATOM | 309 | O | LEU | 57 | 2.934 | −18.393 | 95.383 | 1.00 | 63.31 | A |
| ATOM | 310 | N | ALA | 58 | 1.385 | −17.591 | 96.807 | 1.00 | 62.63 | A |
| ATOM | 311 | CA | ALA | 58 | 2.063 | −18.074 | 98.010 | 1.00 | 61.38 | A |
| ATOM | 312 | CB | ALA | 58 | 1.586 | −17.286 | 99.229 | 1.00 | 60.84 | A |
| ATOM | 313 | C | ALA | 58 | 1.752 | −19.562 | 98.184 | 1.00 | 60.68 | A |
| ATOM | 314 | O | ALA | 58 | 2.385 | −20.261 | 98.979 | 1.00 | 60.38 | A |
| ATOM | 315 | N | ASP | 59 | 0.765 | −20.024 | 97.422 | 1.00 | 59.38 | A |
| ATOM | 316 | CA | ASP | 59 | 0.321 | −21.413 | 97.427 | 1.00 | 57.30 | A |
| ATOM | 317 | CB | ASP | 59 | −1.058 | −21.498 | 96.770 | 1.00 | 58.25 | A |
| ATOM | 318 | CG | ASP | 59 | −1.438 | −22.907 | 96.386 | 1.00 | 58.65 | A |
| ATOM | 319 | OD1 | ASP | 59 | −1.549 | −23.767 | 97.285 | 1.00 | 58.25 | A |
| ATOM | 320 | OD2 | ASP | 59 | −1.628 | −23.151 | 95.175 | 1.00 | 59.46 | A |
| ATOM | 321 | C | ASP | 59 | 1.314 | −22.267 | 96.652 | 1.00 | 55.60 | A |
| ATOM | 322 | O | ASP | 59 | 1.588 | −23.414 | 97.007 | 1.00 | 55.05 | A |
| ATOM | 323 | N | LYS | 60 | 1.849 | −21.681 | 95.587 | 1.00 | 53.85 | A |
| ATOM | 324 | CA | LYS | 60 | 2.819 | −22.340 | 94.718 | 1.00 | 51.83 | A |
| ATOM | 325 | CB | LYS | 60 | 2.099 | −23.322 | 93.787 | 1.00 | 52.01 | A |
| ATOM | 326 | CG | LYS | 60 | 2.982 | −23.940 | 92.720 | 1.00 | 51.22 | A |
| ATOM | 327 | CD | LYS | 60 | 2.184 | −24.835 | 91.795 | 1.00 | 50.89 | A |
| ATOM | 328 | CE | LYS | 60 | 3.054 | −25.341 | 90.663 | 1.00 | 52.06 | A |
| ATOM | 329 | NZ | LYS | 60 | 3.650 | −24.213 | 89.891 | 1.00 | 52.67 | A |
| ATOM | 330 | C | LYS | 60 | 3.534 | −21.258 | 93.900 | 1.00 | 50.64 | A |
| ATOM | 331 | O | LYS | 60 | 2.894 | −20.350 | 93.358 | 1.00 | 51.26 | A |
| ATOM | 332 | N | SER | 61 | 4.855 | −21.347 | 93.805 | 1.00 | 46.71 | A |
| ATOM | 333 | CA | SER | 61 | 5.582 | −20.340 | 93.056 | 1.00 | 42.61 | A |
| ATOM | 334 | CB | SER | 61 | 5.478 | −18.996 | 93.778 | 1.00 | 42.79 | A |
| ATOM | 335 | OG | SER | 61 | 6.132 | −19.048 | 95.039 | 1.00 | 41.39 | A |
| ATOM | 336 | C | SER | 61 | 7.049 | −20.668 | 92.846 | 1.00 | 40.50 | A |
| ATOM | 337 | O | SER | 61 | 7.581 | −21.619 | 93.412 | 1.00 | 39.84 | A |
| ATOM | 338 | N | SER | 62 | 7.691 | −19.856 | 92.017 | 1.00 | 37.70 | A |
| ATOM | 339 | CA | SER | 62 | 9.104 | −19.998 | 91.732 | 1.00 | 34.42 | A |
| ATOM | 340 | CB | SER | 62 | 9.363 | −19.776 | 90.245 | 1.00 | 34.68 | A |
| ATOM | 341 | OG | SER | 62 | 10.742 | −19.881 | 89.964 | 1.00 | 38.74 | A |
| ATOM | 342 | C | SER | 62 | 9.796 | −18.917 | 92.554 | 1.00 | 32.09 | A |
| ATOM | 343 | O | SER | 62 | 9.181 | −17.903 | 92.888 | 1.00 | 29.47 | A |
| ATOM | 344 | N | ARG | 63 | 11.062 | −19.126 | 92.896 | 1.00 | 30.00 | A |
| ATOM | 345 | CA | ARG | 63 | 11.775 | −18.136 | 93.690 | 1.00 | 29.48 | A |
| ATOM | 346 | CB | ARG | 63 | 11.685 | −18.472 | 95.189 | 1.00 | 31.57 | A |
| ATOM | 347 | CG | ARG | 63 | 10.273 | −18.695 | 95.710 | 1.00 | 35.27 | A |
| ATOM | 348 | CD | ARG | 63 | 10.178 | −18.504 | 97.218 | 1.00 | 37.21 | A |
| ATOM | 349 | NE | ARG | 63 | 10.260 | −17.093 | 97.590 | 1.00 | 42.67 | A |
| ATOM | 350 | CZ | ARG | 63 | 9.885 | −16.601 | 98.768 | 1.00 | 44.05 | A |
| ATOM | 351 | NH1 | ARG | 63 | 9.995 | −15.299 | 99.014 | 1.00 | 42.72 | A |
| ATOM | 352 | NH2 | ARG | 63 | 9.394 | −17.408 | 99.700 | 1.00 | 46.01 | A |
| ATOM | 353 | C | ARG | 63 | 13.239 | −17.994 | 93.314 | 1.00 | 27.46 | A |
| ATOM | 354 | O | ARG | 63 | 13.831 | −18.887 | 92.702 | 1.00 | 26.59 | A |
| ATOM | 355 | N | LYS | 64 | 13.807 | −16.853 | 93.693 | 1.00 | 25.59 | A |
| ATOM | 356 | CA | LYS | 64 | 15.216 | −16.539 | 93.467 | 1.00 | 23.77 | A |
| ATOM | 357 | CB | LYS | 64 | 15.353 | −15.299 | 92.587 | 1.00 | 25.43 | A |
| ATOM | 358 | CG | LYS | 64 | 15.991 | −15.532 | 91.231 | 1.00 | 26.32 | A |
| ATOM | 359 | CD | LYS | 64 | 15.095 | −16.338 | 90.323 | 1.00 | 28.26 | A |
| ATOM | 360 | CE | LYS | 64 | 15.692 | −16.456 | 88.925 | 1.00 | 29.50 | A |
| ATOM | 361 | NZ | LYS | 64 | 15.825 | −15.135 | 88.250 | 1.00 | 27.38 | A |
| ATOM | 362 | C | LYS | 64 | 15.808 | −16.257 | 94.854 | 1.00 | 23.10 | A |
| ATOM | 363 | O | LYS | 64 | 15.244 | −15.488 | 95.637 | 1.00 | 22.42 | A |
| ATOM | 364 | N | THR | 65 | 16.943 | −16.876 | 95.154 | 1.00 | 22.03 | A |
| ATOM | 365 | CA | THR | 65 | 17.586 | −16.715 | 96.452 | 1.00 | 20.67 | A |
| ATOM | 366 | CB | THR | 65 | 17.595 | −18.081 | 97.179 | 1.00 | 21.12 | A |
| ATOM | 367 | OG1 | THR | 65 | 16.352 | −18.252 | 97.870 | 1.00 | 22.06 | A |
| ATOM | 368 | CG2 | THR | 65 | 18.740 | −18.187 | 98.154 | 1.00 | 27.20 | A |
| ATOM | 369 | C | THR | 65 | 19.002 | −16.136 | 96.363 | 1.00 | 19.65 | A |
| ATOM | 370 | O | THR | 65 | 19.735 | −16.430 | 95.425 | 1.00 | 22.34 | A |
| ATOM | 371 | N | TYR | 66 | 19.377 | −15.300 | 97.331 | 1.00 | 17.01 | A |
| ATOM | 372 | CA | TYR | 66 | 20.714 | −14.695 | 97.349 | 1.00 | 15.46 | A |
| ATOM | 373 | CB | TYR | 66 | 20.686 | −13.244 | 96.829 | 1.00 | 14.31 | A |
| ATOM | 374 | CG | TYR | 66 | 20.034 | −13.055 | 95.482 | 1.00 | 14.28 | A |
| ATOM | 375 | CD1 | TYR | 66 | 18.651 | −12.984 | 95.366 | 1.00 | 12.32 | A |
| ATOM | 376 | CE1 | TYR | 66 | 18.046 | −12.799 | 94.130 | 1.00 | 14.42 | A |
| ATOM | 377 | CD2 | TYR | 66 | 20.804 | −12.938 | 94.320 | 1.00 | 12.69 | A |
| ATOM | 378 | CE2 | TYR | 66 | 20.207 | −12.752 | 93.079 | 1.00 | 10.53 | A |
| ATOM | 379 | CZ | TYR | 66 | 18.829 | −12.682 | 92.993 | 1.00 | 13.34 | A |
| ATOM | 380 | OH | TYR | 66 | 18.214 | −12.483 | 91.776 | 1.00 | 14.95 | A |
| ATOM | 381 | C | TYR | 66 | 21.298 | −14.675 | 98.754 | 1.00 | 14.50 | A |
| ATOM | 382 | O | TYR | 66 | 20.580 | −14.461 | 99.733 | 1.00 | 13.73 | A |
| ATOM | 383 | N | THR | 67 | 22.605 | −14.880 | 98.854 | 1.00 | 14.35 | A |
| ATOM | 384 | CA | THR | 67 | 23.260 | −14.853 | 100.154 | 1.00 | 15.82 | A |
| ATOM | 385 | CB | THR | 67 | 24.083 | −16.127 | 100.386 | 1.00 | 16.72 | A |
| ATOM | 386 | OG1 | THR | 67 | 23.209 | −17.261 | 100.418 | 1.00 | 17.16 | A |
| ATOM | 387 | CG2 | THR | 67 | 24.845 | −16.045 | 101.698 | 1.00 | 17.80 | A |
| ATOM | 388 | C | THR | 67 | 24.191 | −13.650 | 100.203 | 1.00 | 16.72 | A |
| ATOM | 389 | O | THR | 67 | 24.992 | −13.450 | 99.293 | 1.00 | 17.55 | A |
| ATOM | 390 | N | PHE | 68 | 24.071 | −12.839 | 101.249 | 1.00 | 16.84 | A |
| ATOM | 391 | CA | PHE | 68 | 24.930 | −11.666 | 101.405 | 1.00 | 18.85 | A |
| ATOM | 392 | CB | PHE | 68 | 24.119 | −10.371 | 101.340 | 1.00 | 17.59 | A |
| ATOM | 393 | CG | PHE | 68 | 23.343 | −10.206 | 100.080 | 1.00 | 17.32 | A |
| ATOM | 394 | CD1 | PHE | 68 | 22.105 | −10.823 | 99.926 | 1.00 | 16.89 | A |
| ATOM | 395 | CD2 | PHE | 68 | 23.855 | −9.447 | 99.036 | 1.00 | 17.68 | A |
| ATOM | 396 | CE1 | PHE | 68 | 21.387 | −10.680 | 98.752 | 1.00 | 15.86 | A |
| ATOM | 397 | CE2 | PHE | 68 | 23.144 | −9.296 | 97.852 | 1.00 | 16.89 | A |
| ATOM | 398 | CZ | PHE | 68 | 21.906 | −9.916 | 97.708 | 1.00 | 17.47 | A |
| ATOM | 399 | C | PHE | 68 | 25.641 | −11.731 | 102.745 | 1.00 | 19.38 | A |
| ATOM | 400 | O | PHE | 68 | 25.505 | −12.703 | 103.479 | 1.00 | 21.74 | A |
| ATOM | 401 | N | ASP | 69 | 26.388 | −10.688 | 103.078 | 1.00 | 19.56 | A |
| ATOM | 402 | CA | ASP | 69 | 27.105 | −10.670 | 104.344 | 1.00 | 20.30 | A |
| ATOM | 403 | CB | ASP | 69 | 28.177 | −9.571 | 104.313 | 1.00 | 20.07 | A |
| ATOM | 404 | CG | ASP | 69 | 29.306 | −9.894 | 103.332 | 1.00 | 22.41 | A |
| ATOM | 405 | OD1 | ASP | 69 | 29.245 | −9.494 | 102.143 | 1.00 | 20.37 | A |
| ATOM | 406 | OD2 | ASP | 69 | 30.259 | −10.582 | 103.756 | 1.00 | 27.46 | A |
| ATOM | 407 | C | ASP | 69 | 26.150 | −10.500 | 105.531 | 1.00 | 20.55 | A |
| ATOM | 408 | O | ASP | 69 | 26.369 | −11.073 | 106.600 | 1.00 | 20.31 | A |
| ATOM | 409 | N | MET | 70 | 25.091 | −9.718 | 105.325 | 1.00 | 21.04 | A |
| ATOM | 410 | CA | MET | 70 | 24.065 | −9.469 | 106.338 | 1.00 | 20.59 | A |
| ATOM | 411 | CB | MET | 70 | 24.464 | −8.322 | 107.257 | 1.00 | 23.87 | A |
| ATOM | 412 | CG | MET | 70 | 25.600 | −8.650 | 108.202 | 1.00 | 27.55 | A |
| ATOM | 413 | SD | MET | 70 | 25.794 | −7.359 | 109.420 | 1.00 | 28.63 | A |
| ATOM | 414 | CE | MET | 70 | 24.665 | −7.914 | 110.676 | 1.00 | 29.22 | A |
| ATOM | 415 | C | MET | 70 | 22.737 | −9.115 | 105.678 | 1.00 | 20.50 | A |
| ATOM | 416 | O | MET | 70 | 22.697 | −8.426 | 104.657 | 1.00 | 19.82 | A |
| ATOM | 417 | N | VAL | 71 | 21.646 | −9.593 | 106.258 | 1.00 | 18.11 | A |
| ATOM | 418 | CA | VAL | 71 | 20.335 | −9.289 | 105.713 | 1.00 | 17.48 | A |
| ATOM | 419 | CB | VAL | 71 | 19.701 | −10.516 | 105.021 | 1.00 | 17.16 | A |
| ATOM | 420 | CG1 | VAL | 71 | 20.532 | −10.915 | 103.802 | 1.00 | 14.56 | A |
| ATOM | 421 | CG2 | VAL | 71 | 19.625 | −11.662 | 105.986 | 1.00 | 19.68 | A |
| ATOM | 422 | C | VAL | 71 | 19.424 | −8.791 | 106.822 | 1.00 | 16.09 | A |
| ATOM | 423 | O | VAL | 71 | 19.395 | −9.350 | 107.913 | 1.00 | 14.72 | A |
| ATOM | 424 | N | PHE | 72 | 18.714 | −7.706 | 106.529 | 1.00 | 16.25 | A |
| ATOM | 425 | CA | PHE | 72 | 17.793 | −7.075 | 107.460 | 1.00 | 15.53 | A |
| ATOM | 426 | CB | PHE | 72 | 18.289 | −5.670 | 107.799 | 1.00 | 14.92 | A |
| ATOM | 427 | CG | PHE | 72 | 19.575 | −5.658 | 108.575 | 1.00 | 17.03 | A |
| ATOM | 428 | CD1 | PHE | 72 | 19.590 | −6.004 | 109.925 | 1.00 | 16.20 | A |
| ATOM | 429 | CD2 | PHE | 72 | 20.782 | −5.332 | 107.950 | 1.00 | 17.34 | A |
| ATOM | 430 | CE1 | PHE | 72 | 20.785 | −6.026 | 110.649 | 1.00 | 16.42 | A |
| ATOM | 431 | CE2 | PHE | 72 | 21.979 | −5.352 | 108.660 | 1.00 | 16.87 | A |
| ATOM | 432 | CZ | PHE | 72 | 21.983 | −5.702 | 110.016 | 1.00 | 16.79 | A |
| ATOM | 433 | C | PHE | 72 | 16.388 | −7.007 | 106.874 | 1.00 | 15.43 | A |
| ATOM | 434 | O | PHE | 72 | 16.163 | −6.394 | 105.834 | 1.00 | 13.98 | A |
| ATOM | 435 | N | GLY | 73 | 15.445 | −7.646 | 107.557 | 1.00 | 18.08 | A |
| ATOM | 436 | CA | GLY | 73 | 14.067 | −7.655 | 107.104 | 1.00 | 17.75 | A |
| ATOM | 437 | C | GLY | 73 | 13.343 | −6.377 | 107.478 | 1.00 | 19.38 | A |
| ATOM | 438 | O | GLY | 73 | 13.918 | −5.477 | 108.101 | 1.00 | 19.14 | A |
| ATOM | 439 | N | ALA | 74 | 12.069 | −6.308 | 107.103 | 1.00 | 20.07 | A |
| ATOM | 440 | CA | ALA | 74 | 11.228 | −5.145 | 107.363 | 1.00 | 20.00 | A |
| ATOM | 441 | CB | ALA | 74 | 9.840 | −5.399 | 106.800 | 1.00 | 19.61 | A |
| ATOM | 442 | C | ALA | 74 | 11.124 | −4.709 | 108.834 | 1.00 | 19.69 | A |
| ATOM | 443 | O | ALA | 74 | 10.972 | −3.525 | 109.123 | 1.00 | 21.06 | A |
| ATOM | 444 | N | SER | 75 | 11.213 | −5.650 | 109.765 | 1.00 | 18.30 | A |
| ATOM | 445 | CA | SER | 75 | 11.103 | −5.300 | 111.177 | 1.00 | 18.31 | A |
| ATOM | 446 | CB | SER | 75 | 10.789 | −6.553 | 111.991 | 1.00 | 16.40 | A |
| ATOM | 447 | OG | SER | 75 | 11.886 | −7.450 | 111.971 | 1.00 | 15.90 | A |
| ATOM | 448 | C | SER | 75 | 12.359 | −4.625 | 111.748 | 1.00 | 18.96 | A |
| ATOM | 449 | O | SER | 75 | 12.368 | −4.196 | 112.902 | 1.00 | 19.99 | A |
| ATOM | 450 | N | THR | 76 | 13.407 | −4.519 | 110.937 | 1.00 | 18.45 | A |
| ATOM | 451 | CA | THR | 76 | 14.667 | −3.932 | 111.390 | 1.00 | 17.88 | A |
| ATOM | 452 | CB | THR | 76 | 15.783 | −4.165 | 110.347 | 1.00 | 18.01 | A |
| ATOM | 453 | OG1 | THR | 76 | 15.861 | −5.567 | 110.019 | 1.00 | 17.20 | A |
| ATOM | 454 | CG2 | THR | 76 | 17.109 | −3.708 | 110.902 | 1.00 | 17.48 | A |
| ATOM | 455 | C | THR | 76 | 14.570 | −2.437 | 111.687 | 1.00 | 17.40 | A |
| ATOM | 456 | O | THR | 76 | 14.064 | −1.667 | 110.877 | 1.00 | 18.84 | A |
| ATOM | 457 | N | LYS | 77 | 15.061 | −2.034 | 112.853 | 1.00 | 16.09 | A |
| ATOM | 458 | CA | LYS | 77 | 15.032 | −0.633 | 113.262 | 1.00 | 17.09 | A |
| ATOM | 459 | CB | LYS | 77 | 14.667 | −0.526 | 114.751 | 1.00 | 19.20 | A |
| ATOM | 460 | CG | LYS | 77 | 13.337 | −1.181 | 115.120 | 1.00 | 20.20 | A |
| ATOM | 461 | CD | LYS | 77 | 12.198 | −0.604 | 114.302 | 1.00 | 24.17 | A |
| ATOM | 462 | CE | LYS | 77 | 10.882 | −1.325 | 114.556 | 1.00 | 28.56 | A |
| ATOM | 463 | NZ | LYS | 77 | 9.741 | −0.673 | 113.832 | 1.00 | 29.29 | A |
| ATOM | 464 | C | LYS | 77 | 16.383 | 0.039 | 113.007 | 1.00 | 16.81 | A |
| ATOM | 465 | O | LYS | 77 | 17.382 | −0.638 | 112.760 | 1.00 | 16.91 | A |
| ATOM | 466 | N | GLN | 78 | 16.414 | 1.368 | 113.067 | 1.00 | 14.39 | A |
| ATOM | 467 | CA | GLN | 78 | 17.657 | 2.101 | 112.831 | 1.00 | 13.21 | A |
| ATOM | 468 | CB | GLN | 78 | 17.422 | 3.611 | 112.945 | 1.00 | 10.26 | A |
| ATOM | 469 | CG | GLN | 78 | 16.343 | 4.179 | 112.017 | 1.00 | 10.24 | A |
| ATOM | 470 | CD | GLN | 78 | 16.799 | 4.325 | 110.579 | 1.00 | 8.85 | A |
| ATOM | 471 | OE1 | GLN | 78 | 17.170 | 3.348 | 109.922 | 1.00 | 10.32 | A |
| ATOM | 472 | NE2 | GLN | 78 | 16.776 | 5.555 | 110.081 | 1.00 | 6.58 | A |
| ATOM | 473 | C | GLN | 78 | 18.750 | 1.687 | 113.821 | 1.00 | 13.02 | A |
| ATOM | 474 | O | GLN | 78 | 19.933 | 1.636 | 113.474 | 1.00 | 11.38 | A |
| ATOM | 475 | N | ILE | 79 | 18.352 | 1.392 | 115.053 | 1.00 | 12.89 | A |
| ATOM | 476 | CA | ILE | 79 | 19.313 | 1.013 | 116.085 | 1.00 | 13.42 | A |
| ATOM | 477 | CB | ILE | 79 | 18.635 | 0.959 | 117.479 | 1.00 | 13.40 | A |
| ATOM | 478 | CG2 | ILE | 79 | 17.591 | −0.142 | 117.508 | 1.00 | 14.83 | A |
| ATOM | 479 | CG1 | ILE | 79 | 19.684 | 0.733 | 118.571 | 1.00 | 13.65 | A |
| ATOM | 480 | CD1 | ILE | 79 | 20.653 | 1.906 | 118.775 | 1.00 | 14.47 | A |
| ATOM | 481 | C | ILE | 79 | 19.972 | −0.329 | 115.771 | 1.00 | 12.91 | A |
| ATOM | 482 | O | ILE | 79 | 21.157 | −0.522 | 116.044 | 1.00 | 12.01 | A |
| ATOM | 483 | N | ASP | 80 | 19.204 | −1.243 | 115.182 | 1.00 | 13.40 | A |
| ATOM | 484 | CA | ASP | 80 | 19.719 | −2.555 | 114.815 | 1.00 | 14.93 | A |
| ATOM | 485 | CB | ASP | 80 | 18.581 | −3.461 | 114.303 | 1.00 | 17.57 | A |
| ATOM | 486 | CG | ASP | 80 | 17.428 | −3.593 | 115.300 | 1.00 | 20.41 | A |
| ATOM | 487 | OD1 | ASP | 80 | 17.692 | −3.811 | 116.504 | 1.00 | 22.08 | A |
| ATOM | 488 | OD2 | ASP | 80 | 16.253 | −3.492 | 114.879 | 1.00 | 21.37 | A |
| ATOM | 489 | C | ASP | 80 | 20.777 | −2.393 | 113.719 | 1.00 | 15.46 | A |
| ATOM | 490 | O | ASP | 80 | 21.845 | −3.007 | 113.769 | 1.00 | 15.07 | A |
| ATOM | 491 | N | VAL | 81 | 20.467 | −1.560 | 112.730 | 1.00 | 15.97 | A |
| ATOM | 492 | CA | VAL | 81 | 21.380 | −1.307 | 111.625 | 1.00 | 16.25 | A |
| ATOM | 493 | CB | VAL | 81 | 20.747 | −0.360 | 110.555 | 1.00 | 16.07 | A |
| ATOM | 494 | CG1 | VAL | 81 | 21.787 | 0.027 | 109.526 | 1.00 | 14.56 | A |
| ATOM | 495 | CG2 | VAL | 81 | 19.568 | −1.049 | 109.857 | 1.00 | 14.48 | A |
| ATOM | 496 | C | VAL | 81 | 22.667 | −0.681 | 112.142 | 1.00 | 18.57 | A |
| ATOM | 497 | O | VAL | 81 | 23.758 | −1.079 | 111.733 | 1.00 | 20.96 | A |
| ATOM | 498 | N | TYR | 82 | 22.549 | 0.289 | 113.046 | 1.00 | 19.05 | A |
| ATOM | 499 | CA | TYR | 82 | 23.732 | 0.946 | 113.583 | 1.00 | 20.41 | A |
| ATOM | 500 | CB | TYR | 82 | 23.339 | 2.132 | 114.471 | 1.00 | 23.17 | A |
| ATOM | 501 | CG | TYR | 82 | 24.532 | 2.903 | 114.992 | 1.00 | 24.73 | A |
| ATOM | 502 | CD1 | TYR | 82 | 25.137 | 2.556 | 116.198 | 1.00 | 24.58 | A |
| ATOM | 503 | CE1 | TYR | 82 | 26.284 | 3.200 | 116.638 | 1.00 | 25.15 | A |
| ATOM | 504 | CD2 | TYR | 82 | 25.107 | 3.928 | 114.237 | 1.00 | 25.38 | A |
| ATOM | 505 | CE2 | TYR | 82 | 26.258 | 4.576 | 114.668 | 1.00 | 25.61 | A |
| ATOM | 506 | CZ | TYR | 82 | 26.842 | 4.204 | 115.868 | 1.00 | 25.89 | A |
| ATOM | 507 | OH | TYR | 82 | 28.000 | 4.818 | 116.297 | 1.00 | 26.74 | A |
| ATOM | 508 | C | TYR | 82 | 24.633 | −0.002 | 114.375 | 1.00 | 22.16 | A |
| ATOM | 509 | O | TYR | 82 | 25.835 | −0.104 | 114.103 | 1.00 | 22.17 | A |
| ATOM | 510 | N | ARG | 83 | 24.059 | −0.694 | 115.352 | 1.00 | 21.11 | A |
| ATOM | 511 | CA | ARG | 83 | 24.834 | −1.615 | 116.170 | 1.00 | 20.40 | A |
| ATOM | 512 | CB | ARG | 83 | 23.928 | −2.263 | 117.222 | 1.00 | 18.85 | A |
| ATOM | 513 | CG | ARG | 83 | 23.521 | −1.315 | 118.339 | 1.00 | 21.14 | A |
| ATOM | 514 | CD | ARG | 83 | 22.272 | −1.804 | 119.065 | 1.00 | 21.88 | A |
| ATOM | 515 | NE | ARG | 83 | 22.478 | −3.061 | 119.779 | 1.00 | 22.27 | A |
| ATOM | 516 | CZ | ARG | 83 | 23.184 | −3.175 | 120.899 | 1.00 | 23.18 | A |
| ATOM | 517 | NH1 | ARG | 83 | 23.757 | −2.104 | 121.434 | 1.00 | 23.11 | A |
| ATOM | 518 | NH2 | ARG | 83 | 23.308 | −4.356 | 121.490 | 1.00 | 23.57 | A |
| ATOM | 519 | C | ARG | 83 | 25.553 | −2.694 | 115.361 | 1.00 | 19.49 | A |
| ATOM | 520 | O | ARG | 83 | 26.702 | −3.022 | 115.647 | 1.00 | 17.49 | A |
| ATOM | 521 | N | SER | 84 | 24.885 | −3.225 | 114.341 | 1.00 | 19.74 | A |
| ATOM | 522 | CA | SER | 84 | 25.462 | −4.283 | 113.519 | 1.00 | 19.67 | A |
| ATOM | 523 | CB | SER | 84 | 24.359 | −5.135 | 112.888 | 1.00 | 21.49 | A |
| ATOM | 524 | OG | SER | 84 | 23.716 | −5.931 | 113.865 | 1.00 | 28.64 | A |
| ATOM | 525 | C | SER | 84 | 26.419 | −3.859 | 112.426 | 1.00 | 18.56 | A |
| ATOM | 526 | O | SER | 84 | 27.487 | −4.436 | 112.302 | 1.00 | 19.77 | A |
| ATOM | 527 | N | VAL | 85 | 26.058 | −2.866 | 111.624 | 1.00 | 18.63 | A |
| ATOM | 528 | CA | VAL | 85 | 26.949 | −2.470 | 110.542 | 1.00 | 19.52 | A |
| ATOM | 529 | CB | VAL | 85 | 26.161 | −2.241 | 109.222 | 1.00 | 19.26 | A |
| ATOM | 530 | CG1 | VAL | 85 | 25.165 | −3.377 | 109.011 | 1.00 | 20.45 | A |
| ATOM | 531 | CG2 | VAL | 85 | 25.448 | −0.925 | 109.251 | 1.00 | 22.19 | A |
| ATOM | 532 | C | VAL | 85 | 27.828 | −1.252 | 110.810 | 1.00 | 19.41 | A |
| ATOM | 533 | O | VAL | 85 | 29.034 | −1.289 | 110.558 | 1.00 | 19.81 | A |
| ATOM | 534 | N | VAL | 86 | 27.236 | −0.189 | 111.342 | 1.00 | 19.42 | A |
| ATOM | 535 | CA | VAL | 86 | 27.959 | 1.053 | 111.603 | 1.00 | 19.60 | A |
| ATOM | 536 | CB | VAL | 86 | 26.971 | 2.226 | 111.815 | 1.00 | 18.59 | A |
| ATOM | 537 | CG1 | VAL | 86 | 27.724 | 3.545 | 111.800 | 1.00 | 19.00 | A |
| ATOM | 538 | CG2 | VAL | 86 | 25.899 | 2.208 | 110.736 | 1.00 | 18.56 | A |
| ATOM | 539 | C | VAL | 86 | 28.950 | 1.067 | 112.773 | 1.00 | 20.31 | A |
| ATOM | 540 | O | VAL | 86 | 30.060 | 1.584 | 112.637 | 1.00 | 19.36 | A |
| ATOM | 541 | N | CYS | 87 | 28.559 | 0.519 | 113.919 | 1.00 | 21.30 | A |
| ATOM | 542 | CA | CYS | 87 | 29.438 | 0.535 | 115.082 | 1.00 | 23.03 | A |
| ATOM | 543 | CB | CYS | 87 | 28.777 | −0.187 | 116.254 | 1.00 | 26.09 | A |
| ATOM | 544 | SG | CYS | 87 | 29.481 | 0.238 | 117.859 | 1.00 | 36.72 | A |
| ATOM | 545 | C | CYS | 87 | 30.824 | −0.056 | 114.804 | 1.00 | 21.77 | A |
| ATOM | 546 | O | CYS | 87 | 31.835 | 0.546 | 115.145 | 1.00 | 21.30 | A |
| ATOM | 547 | N | PRO | 88 | 30.894 | −1.241 | 114.185 | 1.00 | 20.49 | A |
| ATOM | 548 | CD | PRO | 88 | 29.856 | −2.240 | 113.881 | 1.00 | 20.97 | A |
| ATOM | 549 | CA | PRO | 88 | 32.231 | −1.783 | 113.926 | 1.00 | 20.97 | A |
| ATOM | 550 | CB | PRO | 88 | 31.948 | −3.215 | 113.473 | 1.00 | 18.41 | A |
| ATOM | 551 | CG | PRO | 88 | 30.571 | −3.133 | 112.895 | 1.00 | 20.02 | A |
| ATOM | 552 | C | PRO | 88 | 33.052 | −0.988 | 112.905 | 1.00 | 21.87 | A |
| ATOM | 553 | O | PRO | 88 | 34.280 | −0.937 | 113.000 | 1.00 | 22.69 | A |
| ATOM | 554 | N | ILE | 89 | 32.380 | −0.373 | 111.934 | 1.00 | 21.27 | A |
| ATOM | 555 | CA | ILE | 89 | 33.068 | 0.417 | 110.915 | 1.00 | 20.39 | A |
| ATOM | 556 | CB | ILE | 89 | 32.130 | 0.723 | 109.720 | 1.00 | 20.42 | A |
| ATOM | 557 | CG2 | ILE | 89 | 32.791 | 1.710 | 108.762 | 1.00 | 16.94 | A |
| ATOM | 558 | CG1 | ILE | 89 | 31.786 | −0.584 | 108.998 | 1.00 | 20.17 | A |
| ATOM | 559 | CD1 | ILE | 89 | 30.749 | −0.429 | 107.886 | 1.00 | 21.44 | A |
| ATOM | 560 | C | ILE | 89 | 33.577 | 1.724 | 111.515 | 1.00 | 21.10 | A |
| ATOM | 561 | O | ILE | 89 | 34.640 | 2.214 | 111.144 | 1.00 | 22.45 | A |
| ATOM | 562 | N | LEU | 90 | 32.818 | 2.287 | 112.449 | 1.00 | 20.96 | A |
| ATOM | 563 | CA | LEU | 90 | 33.229 | 3.522 | 113.103 | 1.00 | 20.72 | A |
| ATOM | 564 | CB | LEU | 90 | 32.086 | 4.094 | 113.940 | 1.00 | 18.19 | A |
| ATOM | 565 | CG | LEU | 90 | 32.407 | 5.390 | 114.687 | 1.00 | 19.36 | A |
| ATOM | 566 | CD1 | LEU | 90 | 32.779 | 6.495 | 113.702 | 1.00 | 17.91 | A |
| ATOM | 567 | CD2 | LEU | 90 | 31.203 | 5.799 | 115.515 | 1.00 | 19.74 | A |
| ATOM | 568 | C | LEU | 90 | 34.443 | 3.248 | 113.989 | 1.00 | 21.43 | A |
| ATOM | 569 | O | LEU | 90 | 35.346 | 4.081 | 114.089 | 1.00 | 22.10 | A |
| ATOM | 570 | N | ASP | 91 | 34.471 | 2.084 | 114.632 | 1.00 | 21.61 | A |
| ATOM | 571 | CA | ASP | 91 | 35.611 | 1.731 | 115.476 | 1.00 | 22.75 | A |
| ATOM | 572 | CB | ASP | 91 | 35.404 | 0.380 | 116.172 | 1.00 | 22.67 | A |
| ATOM | 573 | CG | ASP | 91 | 34.535 | 0.486 | 117.410 | 1.00 | 25.39 | A |
| ATOM | 574 | OD1 | ASP | 91 | 34.386 | 1.604 | 117.947 | 1.00 | 24.95 | A |
| ATOM | 575 | OD2 | ASP | 91 | 34.006 | −0.552 | 117.859 | 1.00 | 27.30 | A |
| ATOM | 576 | C | ASP | 91 | 36.877 | 1.667 | 114.618 | 1.00 | 22.42 | A |
| ATOM | 577 | O | ASP | 91 | 37.956 | 2.039 | 115.077 | 1.00 | 20.39 | A |
| ATOM | 578 | N | GLU | 92 | 36.749 | 1.199 | 113.378 | 1.00 | 20.58 | A |
| ATOM | 579 | CA | GLU | 92 | 37.907 | 1.130 | 112.499 | 1.00 | 22.88 | A |
| ATOM | 580 | CB | GLU | 92 | 37.599 | 0.311 | 111.238 | 1.00 | 24.90 | A |
| ATOM | 581 | CG | GLU | 92 | 38.131 | −1.120 | 111.282 | 1.00 | 31.75 | A |
| ATOM | 582 | CD | GLU | 92 | 38.517 | −1.655 | 109.902 | 1.00 | 35.40 | A |
| ATOM | 583 | OE1 | GLU | 92 | 39.330 | −1.007 | 109.203 | 1.00 | 36.87 | A |
| ATOM | 584 | OE2 | GLU | 92 | 38.017 | −2.732 | 109.519 | 1.00 | 37.95 | A |
| ATOM | 585 | C | GLU | 92 | 38.358 | 2.537 | 112.100 | 1.00 | 22.24 | A |
| ATOM | 586 | O | GLU | 92 | 39.554 | 2.799 | 111.964 | 1.00 | 21.80 | A |
| ATOM | 587 | N | VAL | 93 | 37.398 | 3.438 | 111.909 | 1.00 | 20.21 | A |
| ATOM | 588 | CA | VAL | 93 | 37.712 | 4.808 | 111.532 | 1.00 | 18.97 | A |
| ATOM | 589 | CB | VAL | 93 | 36.422 | 5.626 | 111.228 | 1.00 | 17.93 | A |
| ATOM | 590 | CG1 | VAL | 93 | 36.755 | 7.102 | 111.094 | 1.00 | 14.46 | A |
| ATOM | 591 | CG2 | VAL | 93 | 35.781 | 5.124 | 109.937 | 1.00 | 16.29 | A |
| ATOM | 592 | C | VAL | 93 | 38.489 | 5.482 | 112.657 | 1.00 | 19.09 | A |
| ATOM | 593 | O | VAL | 93 | 39.477 | 6.174 | 112.414 | 1.00 | 18.02 | A |
| ATOM | 594 | N | ILE | 94 | 38.044 | 5.263 | 113.889 | 1.00 | 19.70 | A |
| ATOM | 595 | CA | ILE | 94 | 38.690 | 5.845 | 115.056 | 1.00 | 21.90 | A |
| ATOM | 596 | CB | ILE | 94 | 37.815 | 5.615 | 116.317 | 1.00 | 22.69 | A |
| ATOM | 597 | CG2 | ILE | 94 | 38.519 | 6.128 | 117.571 | 1.00 | 22.60 | A |
| ATOM | 598 | CG1 | ILE | 94 | 36.472 | 6.336 | 116.124 | 1.00 | 22.49 | A |
| ATOM | 599 | CD1 | ILE | 94 | 35.480 | 6.155 | 117.266 | 1.00 | 22.50 | A |
| ATOM | 600 | C | ILE | 94 | 40.116 | 5.302 | 115.265 | 1.00 | 24.26 | A |
| ATOM | 601 | O | ILE | 94 | 40.924 | 5.931 | 115.945 | 1.00 | 24.34 | A |
| ATOM | 602 | N | MET | 95 | 40.428 | 4.148 | 114.672 | 1.00 | 25.73 | A |
| ATOM | 603 | CA | MET | 95 | 41.767 | 3.559 | 114.777 | 1.00 | 27.17 | A |
| ATOM | 604 | CB | MET | 95 | 41.732 | 2.047 | 114.532 | 1.00 | 29.33 | A |
| ATOM | 605 | CG | MET | 95 | 41.102 | 1.237 | 115.643 | 1.00 | 35.68 | A |
| ATOM | 606 | SD | MET | 95 | 41.281 | −0.526 | 115.337 | 1.00 | 44.01 | A |
| ATOM | 607 | CE | MET | 95 | 39.718 | −0.911 | 114.541 | 1.00 | 39.10 | A |
| ATOM | 608 | C | MET | 95 | 42.722 | 4.183 | 113.761 | 1.00 | 27.37 | A |
| ATOM | 609 | O | MET | 95 | 43.907 | 3.832 | 113.711 | 1.00 | 26.10 | A |
| ATOM | 610 | N | GLY | 96 | 42.197 | 5.088 | 112.939 | 1.00 | 26.75 | A |
| ATOM | 611 | CA | GLY | 96 | 43.020 | 5.753 | 111.941 | 1.00 | 26.52 | A |
| ATOM | 612 | C | GLY | 96 | 42.861 | 5.220 | 110.529 | 1.00 | 25.69 | A |
| ATOM | 613 | O | GLY | 96 | 43.752 | 5.373 | 109.690 | 1.00 | 25.52 | A |
| ATOM | 614 | N | TYR | 97 | 41.720 | 4.597 | 110.264 | 1.00 | 25.64 | A |
| ATOM | 615 | CA | TYR | 97 | 41.439 | 4.033 | 108.949 | 1.00 | 24.96 | A |
| ATOM | 616 | CB | TYR | 97 | 40.932 | 2.592 | 109.113 | 1.00 | 29.74 | A |
| ATOM | 617 | CG | TYR | 97 | 42.007 | 1.569 | 109.444 | 1.00 | 34.33 | A |
| ATOM | 618 | CD1 | TYR | 97 | 42.993 | 1.243 | 108.514 | 1.00 | 36.66 | A |
| ATOM | 619 | CE1 | TYR | 97 | 43.970 | 0.292 | 108.798 | 1.00 | 39.73 | A |
| ATOM | 620 | CD2 | TYR | 97 | 42.025 | 0.914 | 110.680 | 1.00 | 35.77 | A |
| ATOM | 621 | CE2 | TYR | 97 | 42.998 | −0.037 | 110.979 | 1.00 | 38.01 | A |
| ATOM | 622 | CZ | TYR | 97 | 43.969 | −0.342 | 110.033 | 1.00 | 40.42 | A |
| ATOM | 623 | OH | TYR | 97 | 44.956 | −1.264 | 110.325 | 1.00 | 41.65 | A |
| ATOM | 624 | C | TYR | 97 | 40.407 | 4.854 | 108.163 | 1.00 | 22.65 | A |
| ATOM | 625 | O | TYR | 97 | 39.749 | 5.741 | 108.711 | 1.00 | 22.45 | A |
| ATOM | 626 | N | ASN | 98 | 40.290 | 4.565 | 106.872 | 1.00 | 19.89 | A |
| ATOM | 627 | CA | ASN | 98 | 39.312 | 5.226 | 106.021 | 1.00 | 18.57 | A |
| ATOM | 628 | CB | ASN | 98 | 39.941 | 5.682 | 104.702 | 1.00 | 19.70 | A |
| ATOM | 629 | CG | ASN | 98 | 40.867 | 6.863 | 104.873 | 1.00 | 21.50 | A |
| ATOM | 630 | OD1 | ASN | 98 | 40.543 | 7.826 | 105.574 | 1.00 | 23.29 | A |
| ATOM | 631 | ND2 | ASN | 98 | 42.020 | 6.807 | 104.222 | 1.00 | 20.02 | A |
| ATOM | 632 | C | ASN | 98 | 38.195 | 4.230 | 105.713 | 1.00 | 18.68 | A |
| ATOM | 633 | O | ASN | 98 | 38.459 | 3.087 | 105.346 | 1.00 | 16.93 | A |
| ATOM | 634 | N | CYS | 99 | 36.949 | 4.657 | 105.865 | 1.00 | 18.23 | A |
| ATOM | 635 | CA | CYS | 99 | 35.825 | 3.776 | 105.575 | 1.00 | 17.76 | A |
| ATOM | 636 | CB | CYS | 99 | 35.244 | 3.186 | 106.867 | 1.00 | 18.42 | A |
| ATOM | 637 | SG | CYS | 99 | 36.378 | 2.095 | 107.771 | 1.00 | 19.49 | A |
| ATOM | 638 | C | CYS | 99 | 34.727 | 4.481 | 104.790 | 1.00 | 15.84 | A |
| ATOM | 639 | O | CYS | 99 | 34.508 | 5.685 | 104.920 | 1.00 | 13.06 | A |
| ATOM | 640 | N | THR | 100 | 34.044 | 3.696 | 103.968 | 1.00 | 15.18 | A |
| ATOM | 641 | CA | THR | 100 | 32.968 | 4.190 | 103.130 | 1.00 | 14.06 | A |
| ATOM | 642 | CB | THR | 100 | 33.417 | 4.278 | 101.657 | 1.00 | 12.78 | A |
| ATOM | 643 | OG1 | THR | 100 | 34.485 | 5.223 | 101.539 | 1.00 | 14.13 | A |
| ATOM | 644 | CG2 | THR | 100 | 32.262 | 4.717 | 100.773 | 1.00 | 12.44 | A |
| ATOM | 645 | C | THR | 100 | 31.759 | 3.260 | 103.200 | 1.00 | 14.15 | A |
| ATOM | 646 | O | THR | 100 | 31.907 | 2.034 | 103.263 | 1.00 | 13.80 | A |
| ATOM | 647 | N | ILE | 101 | 30.568 | 3.851 | 103.199 | 1.00 | 12.37 | A |
| ATOM | 648 | CA | ILE | 101 | 29.329 | 3.088 | 103.202 | 1.00 | 11.07 | A |
| ATOM | 649 | CB | ILE | 101 | 28.608 | 3.158 | 104.551 | 1.00 | 10.99 | A |
| ATOM | 650 | CG2 | ILE | 101 | 27.404 | 2.213 | 104.527 | 1.00 | 11.07 | A |
| ATOM | 651 | CG1 | ILE | 101 | 29.551 | 2.756 | 105.682 | 1.00 | 11.36 | A |
| ATOM | 652 | CD1 | ILE | 101 | 28.880 | 2.767 | 107.071 | 1.00 | 11.31 | A |
| ATOM | 653 | C | ILE | 101 | 28.394 | 3.659 | 102.123 | 1.00 | 10.34 | A |
| ATOM | 654 | O | ILE | 101 | 28.077 | 4.842 | 102.133 | 1.00 | 8.62 | A |
| ATOM | 655 | N | PHE | 102 | 27.980 | 2.807 | 101.192 | 1.00 | 8.88 | A |
| ATOM | 656 | CA | PHE | 102 | 27.089 | 3.200 | 100.113 | 1.00 | 8.18 | A |
| ATOM | 657 | CB | PHE | 102 | 27.521 | 2.554 | 98.798 | 1.00 | 8.39 | A |
| ATOM | 658 | CG | PHE | 102 | 28.786 | 3.107 | 98.212 | 1.00 | 8.44 | A |
| ATOM | 659 | CD1 | PHE | 102 | 28.746 | 4.237 | 97.400 | 1.00 | 8.21 | A |
| ATOM | 660 | CD2 | PHE | 102 | 30.004 | 2.449 | 98.402 | 1.00 | 7.42 | A |
| ATOM | 661 | CE1 | PHE | 102 | 29.901 | 4.712 | 96.770 | 1.00 | 10.64 | A |
| ATOM | 662 | CE2 | PHE | 102 | 31.167 | 2.910 | 97.780 | 1.00 | 9.88 | A |
| ATOM | 663 | CZ | PHE | 102 | 31.119 | 4.044 | 96.957 | 1.00 | 10.26 | A |
| ATOM | 664 | C | PHE | 102 | 25.686 | 2.695 | 100.418 | 1.00 | 9.34 | A |
| ATOM | 665 | O | PHE | 102 | 25.514 | 1.676 | 101.084 | 1.00 | 9.83 | A |
| ATOM | 666 | N | ALA | 103 | 24.686 | 3.420 | 99.937 | 1.00 | 8.83 | A |
| ATOM | 667 | CA | ALA | 103 | 23.301 | 3.008 | 100.088 | 1.00 | 6.41 | A |
| ATOM | 668 | CB | ALA | 103 | 22.503 | 4.057 | 100.836 | 1.00 | 6.59 | A |
| ATOM | 669 | C | ALA | 103 | 22.887 | 2.920 | 98.619 | 1.00 | 5.06 | A |
| ATOM | 670 | O | ALA | 103 | 22.988 | 3.898 | 97.890 | 1.00 | 3.08 | A |
| ATOM | 671 | N | TYR | 104 | 22.476 | 1.735 | 98.184 | 1.00 | 4.26 | A |
| ATOM | 672 | CA | TYR | 104 | 22.110 | 1.498 | 96.791 | 1.00 | 4.91 | A |
| ATOM | 673 | CB | TYR | 104 | 23.142 | 0.552 | 96.137 | 1.00 | 3.89 | A |
| ATOM | 674 | CG | TYR | 104 | 22.911 | 0.238 | 94.666 | 1.00 | 4.19 | A |
| ATOM | 675 | CD1 | TYR | 104 | 21.933 | −0.675 | 94.260 | 1.00 | 6.04 | A |
| ATOM | 676 | CE1 | TYR | 104 | 21.722 | −0.946 | 92.898 | 1.00 | 7.93 | A |
| ATOM | 677 | CD2 | TYR | 104 | 23.667 | 0.868 | 93.679 | 1.00 | 5.77 | A |
| ATOM | 678 | CE2 | TYR | 104 | 23.466 | 0.608 | 92.326 | 1.00 | 5.74 | A |
| ATOM | 679 | CZ | TYR | 104 | 22.500 | −0.295 | 91.944 | 1.00 | 6.93 | A |
| ATOM | 680 | OH | TYR | 104 | 22.326 | −0.551 | 90.604 | 1.00 | 8.61 | A |
| ATOM | 681 | C | TYR | 104 | 20.718 | 0.893 | 96.678 | 1.00 | 5.23 | A |
| ATOM | 682 | O | TYR | 104 | 20.346 | 0.007 | 97.445 | 1.00 | 7.02 | A |
| ATOM | 683 | N | GLY | 105 | 19.955 | 1.368 | 95.704 | 1.00 | 3.82 | A |
| ATOM | 684 | CA | GLY | 105 | 18.620 | 0.857 | 95.521 | 1.00 | 5.02 | A |
| ATOM | 685 | C | GLY | 105 | 17.705 | 1.803 | 94.773 | 1.00 | 5.87 | A |
| ATOM | 686 | O | GLY | 105 | 17.981 | 2.992 | 94.590 | 1.00 | 6.06 | A |
| ATOM | 687 | N | GLN | 106 | 16.598 | 1.244 | 94.326 | 1.00 | 4.13 | A |
| ATOM | 688 | CA | GLN | 106 | 15.601 | 1.986 | 93.591 | 1.00 | 6.44 | A |
| ATOM | 689 | CB | GLN | 106 | 14.513 | 0.998 | 93.158 | 1.00 | 6.41 | A |
| ATOM | 690 | CG | GLN | 106 | 13.175 | 1.585 | 92.817 | 1.00 | 11.96 | A |
| ATOM | 691 | CD | GLN | 106 | 12.136 | 0.511 | 92.499 | 1.00 | 14.57 | A |
| ATOM | 692 | OE1 | GLN | 106 | 12.060 | −0.539 | 93.172 | 1.00 | 12.16 | A |
| ATOM | 693 | NE2 | GLN | 106 | 11.318 | 0.774 | 91.483 | 1.00 | 10.80 | A |
| ATOM | 694 | C | GLN | 106 | 15.047 | 3.091 | 94.488 | 1.00 | 7.89 | A |
| ATOM | 695 | O | GLN | 106 | 15.083 | 2.992 | 95.725 | 1.00 | 8.30 | A |
| ATOM | 696 | N | THR | 107 | 14.558 | 4.157 | 93.869 | 1.00 | 8.49 | A |
| ATOM | 697 | CA | THR | 107 | 13.981 | 5.259 | 94.620 | 1.00 | 8.83 | A |
| ATOM | 698 | CB | THR | 107 | 13.532 | 6.371 | 93.668 | 1.00 | 10.17 | A |
| ATOM | 699 | OG1 | THR | 107 | 14.681 | 6.936 | 93.023 | 1.00 | 11.92 | A |
| ATOM | 700 | CG2 | THR | 107 | 12.783 | 7.464 | 94.431 | 1.00 | 9.05 | A |
| ATOM | 701 | C | THR | 107 | 12.763 | 4.751 | 95.392 | 1.00 | 11.60 | A |
| ATOM | 702 | O | THR | 107 | 11.936 | 4.017 | 94.838 | 1.00 | 13.74 | A |
| ATOM | 703 | N | GLY | 108 | 12.661 | 5.121 | 96.668 | 1.00 | 11.74 | A |
| ATOM | 704 | CA | GLY | 108 | 11.527 | 4.703 | 97.476 | 1.00 | 9.99 | A |
| ATOM | 705 | C | GLY | 108 | 11.738 | 3.461 | 98.330 | 1.00 | 11.25 | A |
| ATOM | 706 | O | GLY | 108 | 10.812 | 3.004 | 99.018 | 1.00 | 12.52 | A |
| ATOM | 707 | N | THR | 109 | 12.947 | 2.915 | 98.313 | 1.00 | 9.04 | A |
| ATOM | 708 | CA | THR | 109 | 13.216 | 1.716 | 99.090 | 1.00 | 8.13 | A |
| ATOM | 709 | CB | THR | 109 | 14.053 | 0.703 | 98.291 | 1.00 | 8.11 | A |
| ATOM | 710 | OG1 | THR | 109 | 15.274 | 1.321 | 97.857 | 1.00 | 5.32 | A |
| ATOM | 711 | CG2 | THR | 109 | 13.269 | 0.220 | 97.079 | 1.00 | 2.18 | A |
| ATOM | 712 | C | THR | 109 | 13.914 | 1.990 | 100.405 | 1.00 | 8.77 | A |
| ATOM | 713 | O | THR | 109 | 14.029 | 1.085 | 101.236 | 1.00 | 9.56 | A |
| ATOM | 714 | N | GLY | 110 | 14.411 | 3.211 | 100.599 | 1.00 | 6.93 | A |
| ATOM | 715 | CA | GLY | 110 | 15.037 | 3.517 | 101.878 | 1.00 | 7.00 | A |
| ATOM | 716 | C | GLY | 110 | 16.491 | 3.959 | 101.985 | 1.00 | 8.39 | A |
| ATOM | 717 | O | GLY | 110 | 17.052 | 3.953 | 103.089 | 1.00 | 6.64 | A |
| ATOM | 718 | N | LYS | 111 | 17.106 | 4.346 | 100.869 | 1.00 | 8.77 | A |
| ATOM | 719 | CA | LYS | 111 | 18.493 | 4.798 | 100.888 | 1.00 | 8.41 | A |
| ATOM | 720 | CB | LYS | 111 | 18.938 | 5.257 | 99.495 | 1.00 | 9.46 | A |
| ATOM | 721 | CG | LYS | 111 | 19.086 | 4.134 | 98.462 | 1.00 | 8.41 | A |
| ATOM | 722 | CD | LYS | 111 | 19.650 | 4.651 | 97.133 | 1.00 | 7.10 | A |
| ATOM | 723 | CE | LYS | 111 | 18.772 | 5.741 | 96.526 | 1.00 | 8.55 | A |
| ATOM | 724 | NZ | LYS | 111 | 17.364 | 5.298 | 96.325 | 1.00 | 7.14 | A |
| ATOM | 725 | C | LYS | 111 | 18.643 | 5.956 | 101.862 | 1.00 | 8.34 | A |
| ATOM | 726 | O | LYS | 111 | 19.448 | 5.895 | 102.789 | 1.00 | 9.08 | A |
| ATOM | 727 | N | THR | 112 | 17.851 | 7.006 | 101.651 | 1.00 | 8.83 | A |
| ATOM | 728 | CA | THR | 112 | 17.896 | 8.198 | 102.502 | 1.00 | 7.73 | A |
| ATOM | 729 | CB | THR | 112 | 17.027 | 9.342 | 101.903 | 1.00 | 8.07 | A |
| ATOM | 730 | OG1 | THR | 112 | 17.347 | 9.520 | 100.502 | 1.00 | 8.01 | A |
| ATOM | 731 | CG2 | THR | 112 | 17.287 | 10.650 | 102.650 | 1.00 | 4.02 | A |
| ATOM | 732 | C | THR | 112 | 17.454 | 7.905 | 103.945 | 1.00 | 8.81 | A |
| ATOM | 733 | O | THR | 112 | 17.997 | 8.458 | 104.894 | 1.00 | 8.08 | A |
| ATOM | 734 | N | PHE | 113 | 16.476 | 7.025 | 104.114 | 1.00 | 11.03 | A |
| ATOM | 735 | CA | PHE | 113 | 16.008 | 6.664 | 105.448 | 1.00 | 11.19 | A |
| ATOM | 736 | CB | PHE | 113 | 14.806 | 5.727 | 105.361 | 1.00 | 10.34 | A |
| ATOM | 737 | CG | PHE | 113 | 14.208 | 5.385 | 106.699 | 1.00 | 10.76 | A |
| ATOM | 738 | CD1 | PHE | 113 | 13.247 | 6.214 | 107.276 | 1.00 | 9.64 | A |
| ATOM | 739 | CD2 | PHE | 113 | 14.623 | 4.249 | 107.393 | 1.00 | 9.33 | A |
| ATOM | 740 | CE1 | PHE | 113 | 12.703 | 5.917 | 108.523 | 1.00 | 10.99 | A |
| ATOM | 741 | CE2 | PHE | 113 | 14.084 | 3.942 | 108.646 | 1.00 | 11.97 | A |
| ATOM | 742 | CZ | PHE | 113 | 13.120 | 4.781 | 109.212 | 1.00 | 9.40 | A |
| ATOM | 743 | C | PHE | 113 | 17.120 | 5.943 | 106.205 | 1.00 | 11.21 | A |
| ATOM | 744 | O | PHE | 113 | 17.254 | 6.081 | 107.418 | 1.00 | 11.83 | A |
| ATOM | 745 | N | THR | 114 | 17.908 | 5.159 | 105.483 | 1.00 | 10.89 | A |
| ATOM | 746 | CA | THR | 114 | 18.992 | 4.422 | 106.101 | 1.00 | 9.91 | A |
| ATOM | 747 | CB | THR | 114 | 19.458 | 3.267 | 105.173 | 1.00 | 12.09 | A |
| ATOM | 748 | OG1 | THR | 114 | 18.375 | 2.336 | 105.001 | 1.00 | 10.83 | A |
| ATOM | 749 | CG2 | THR | 114 | 20.677 | 2.537 | 105.763 | 1.00 | 9.73 | A |
| ATOM | 750 | C | THR | 114 | 20.167 | 5.329 | 106.438 | 1.00 | 10.11 | A |
| ATOM | 751 | O | THR | 114 | 20.650 | 5.328 | 107.569 | 1.00 | 10.60 | A |
| ATOM | 752 | N | MET | 115 | 20.606 | 6.125 | 105.466 | 1.00 | 11.39 | A |
| ATOM | 753 | CA | MET | 115 | 21.745 | 7.021 | 105.666 | 1.00 | 11.76 | A |
| ATOM | 754 | CB | MET | 115 | 22.286 | 7.503 | 104.323 | 1.00 | 14.08 | A |
| ATOM | 755 | CG | MET | 115 | 22.774 | 6.402 | 103.406 | 1.00 | 21.28 | A |
| ATOM | 756 | SD | MET | 115 | 24.093 | 5.411 | 104.142 | 1.00 | 28.02 | A |
| ATOM | 757 | CE | MET | 115 | 25.184 | 6.682 | 104.670 | 1.00 | 16.59 | A |
| ATOM | 758 | C | MET | 115 | 21.489 | 8.240 | 106.547 | 1.00 | 11.39 | A |
| ATOM | 759 | O | MET | 115 | 22.347 | 8.607 | 107.349 | 1.00 | 11.70 | A |
| ATOM | 760 | N | GLU | 116 | 20.322 | 8.868 | 106.410 | 1.00 | 10.32 | A |
| ATOM | 761 | CA | GLU | 116 | 20.023 | 10.064 | 107.197 | 1.00 | 9.04 | A |
| ATOM | 762 | CB | GLU | 116 | 19.498 | 11.185 | 106.299 | 1.00 | 11.83 | A |
| ATOM | 763 | CG | GLU | 116 | 20.215 | 11.349 | 104.970 | 1.00 | 15.21 | A |
| ATOM | 764 | CD | GLU | 116 | 19.911 | 12.682 | 104.319 | 1.00 | 17.70 | A |
| ATOM | 765 | OE1 | GLU | 116 | 18.751 | 13.137 | 104.405 | 1.00 | 20.63 | A |
| ATOM | 766 | OE2 | GLU | 116 | 20.830 | 13.272 | 103.715 | 1.00 | 19.36 | A |
| ATOM | 767 | C | GLU | 116 | 19.021 | 9.867 | 108.319 | 1.00 | 8.57 | A |
| ATOM | 768 | O | GLU | 116 | 19.225 | 10.344 | 109.430 | 1.00 | 6.66 | A |
| ATOM | 769 | N | GLY | 117 | 17.937 | 9.162 | 108.024 | 1.00 | 10.69 | A |
| ATOM | 770 | CA | GLY | 117 | 16.894 | 8.961 | 109.011 | 1.00 | 12.05 | A |
| ATOM | 771 | C | GLY | 117 | 15.906 | 10.119 | 108.921 | 1.00 | 14.49 | A |
| ATOM | 772 | O | GLY | 117 | 16.009 | 10.967 | 108.030 | 1.00 | 15.09 | A |
| ATOM | 773 | N | GLU | 118 | 14.954 | 10.176 | 109.844 | 1.00 | 15.27 | A |
| ATOM | 774 | CA | GLU | 118 | 13.955 | 11.240 | 109.827 | 1.00 | 17.05 | A |
| ATOM | 775 | CB | GLU | 118 | 12.680 | 10.764 | 109.132 | 1.00 | 18.95 | A |
| ATOM | 776 | CG | GLU | 118 | 12.881 | 10.219 | 107.732 | 1.00 | 24.85 | A |
| ATOM | 777 | CD | GLU | 118 | 11.659 | 9.462 | 107.228 | 1.00 | 28.50 | A |
| ATOM | 778 | OE1 | GLU | 118 | 11.639 | 9.064 | 106.047 | 1.00 | 29.02 | A |
| ATOM | 779 | OE2 | GLU | 118 | 10.715 | 9.260 | 108.025 | 1.00 | 31.54 | A |
| ATOM | 780 | C | GLU | 118 | 13.601 | 11.631 | 111.246 | 1.00 | 15.85 | A |
| ATOM | 781 | O | GLU | 118 | 14.159 | 11.111 | 112.206 | 1.00 | 17.29 | A |
| ATOM | 782 | N | ARG | 119 | 12.660 | 12.549 | 111.381 | 1.00 | 14.03 | A |
| ATOM | 783 | CA | ARG | 119 | 12.238 | 12.955 | 112.701 | 1.00 | 12.36 | A |
| ATOM | 784 | CB | ARG | 119 | 12.058 | 14.469 | 112.765 | 1.00 | 9.51 | A |
| ATOM | 785 | CG | ARG | 119 | 13.311 | 15.275 | 112.459 | 1.00 | 9.85 | A |
| ATOM | 786 | CD | ARG | 119 | 14.517 | 14.768 | 113.223 | 1.00 | 9.11 | A |
| ATOM | 787 | NE | ARG | 119 | 14.226 | 14.503 | 114.632 | 1.00 | 11.37 | A |
| ATOM | 788 | CZ | ARG | 119 | 14.274 | 15.409 | 115.601 | 1.00 | 9.83 | A |
| ATOM | 789 | NH1 | ARG | 119 | 14.607 | 16.663 | 115.326 | 1.00 | 8.80 | A |
| ATOM | 790 | NH2 | ARG | 119 | 14.003 | 15.052 | 116.851 | 1.00 | 8.38 | A |
| ATOM | 791 | C | ARG | 119 | 10.909 | 12.278 | 113.012 | 1.00 | 13.30 | A |
| ATOM | 792 | O | ARG | 119 | 10.055 | 12.134 | 112.140 | 1.00 | 12.33 | A |
| ATOM | 793 | N | SER | 120 | 10.746 | 11.819 | 114.244 | 1.00 | 14.08 | A |
| ATOM | 794 | CA | SER | 120 | 9.478 | 11.232 | 114.630 | 1.00 | 14.63 | A |
| ATOM | 795 | CB | SER | 120 | 9.563 | 10.651 | 116.037 | 1.00 | 13.18 | A |
| ATOM | 796 | OG | SER | 120 | 10.380 | 9.500 | 116.043 | 1.00 | 13.75 | A |
| ATOM | 797 | C | SER | 120 | 8.542 | 12.434 | 114.610 | 1.00 | 14.70 | A |
| ATOM | 798 | O | SER | 120 | 8.966 | 13.556 | 114.877 | 1.00 | 14.22 | A |
| ATOM | 799 | N | PRO | 121 | 7.263 | 12.222 | 114.295 | 1.00 | 15.80 | A |
| ATOM | 800 | CD | PRO | 121 | 6.629 | 10.969 | 113.860 | 1.00 | 15.88 | A |
| ATOM | 801 | CA | PRO | 121 | 6.312 | 13.340 | 114.253 | 1.00 | 16.98 | A |
| ATOM | 802 | CB | PRO | 121 | 5.037 | 12.699 | 113.703 | 1.00 | 17.68 | A |
| ATOM | 803 | CG | PRO | 121 | 5.528 | 11.476 | 112.967 | 1.00 | 18.94 | A |
| ATOM | 804 | C | PRO | 121 | 6.036 | 14.035 | 115.589 | 1.00 | 17.31 | A |
| ATOM | 805 | O | PRO | 121 | 6.316 | 13.495 | 116.662 | 1.00 | 17.01 | A |
| ATOM | 806 | N | ASN | 122 | 5.493 | 15.249 | 115.498 | 1.00 | 18.27 | A |
| ATOM | 807 | CA | ASN | 122 | 5.079 | 16.029 | 116.659 | 1.00 | 19.75 | A |
| ATOM | 808 | CB | ASN | 122 | 3.899 | 15.303 | 117.323 | 1.00 | 22.14 | A |
| ATOM | 809 | CG | ASN | 122 | 2.806 | 16.243 | 117.782 | 1.00 | 25.67 | A |
| ATOM | 810 | OD1 | ASN | 122 | 2.331 | 17.090 | 117.020 | 1.00 | 28.24 | A |
| ATOM | 811 | ND2 | ASN | 122 | 2.386 | 16.089 | 119.029 | 1.00 | 29.36 | A |
| ATOM | 812 | C | ASN | 122 | 6.137 | 16.341 | 117.714 | 1.00 | 20.30 | A |
| ATOM | 813 | O | ASN | 122 | 5.810 | 16.490 | 118.889 | 1.00 | 19.52 | A |
| ATOM | 814 | N | GLU | 123 | 7.398 | 16.443 | 117.312 | 1.00 | 20.21 | A |
| ATOM | 815 | CA | GLU | 123 | 8.460 | 16.745 | 118.267 | 1.00 | 21.19 | A |
| ATOM | 816 | CB | GLU | 123 | 8.341 | 18.185 | 118.781 | 1.00 | 20.11 | A |
| ATOM | 817 | CG | GLU | 123 | 8.519 | 19.249 | 117.731 | 1.00 | 20.41 | A |
| ATOM | 818 | CD | GLU | 123 | 8.575 | 20.654 | 118.319 | 1.00 | 21.92 | A |
| ATOM | 819 | OE1 | GLU | 123 | 7.688 | 21.013 | 119.133 | 1.00 | 18.15 | A |
| ATOM | 820 | OE2 | GLU | 123 | 9.507 | 21.404 | 117.951 | 1.00 | 21.94 | A |
| ATOM | 821 | C | GLU | 123 | 8.446 | 15.806 | 119.468 | 1.00 | 21.37 | A |
| ATOM | 822 | O | GLU | 123 | 8.632 | 16.247 | 120.602 | 1.00 | 19.07 | A |
| ATOM | 823 | N | GLU | 124 | 8.226 | 14.518 | 119.233 | 1.00 | 22.79 | A |
| ATOM | 824 | CA | GLU | 124 | 8.210 | 13.577 | 120.339 | 1.00 | 22.88 | A |
| ATOM | 825 | CB | GLU | 124 | 7.685 | 12.215 | 119.887 | 1.00 | 25.26 | A |
| ATOM | 826 | CG | GLU | 124 | 7.600 | 11.205 | 121.033 | 1.00 | 30.44 | A |
| ATOM | 827 | CD | GLU | 124 | 6.924 | 9.899 | 120.636 | 1.00 | 34.84 | A |
| ATOM | 828 | OE1 | GLU | 124 | 6.827 | 9.003 | 121.508 | 1.00 | 33.81 | A |
| ATOM | 829 | OE2 | GLU | 124 | 6.494 | 9.772 | 119.464 | 1.00 | 37.51 | A |
| ATOM | 830 | C | GLU | 124 | 9.592 | 13.404 | 120.964 | 1.00 | 22.45 | A |
| ATOM | 831 | O | GLU | 124 | 9.715 | 13.235 | 122.180 | 1.00 | 23.30 | A |
| ATOM | 832 | N | TYR | 125 | 10.635 | 13.452 | 120.142 | 1.00 | 20.18 | A |
| ATOM | 833 | CA | TYR | 125 | 11.988 | 13.269 | 120.657 | 1.00 | 19.15 | A |
| ATOM | 834 | CB | TYR | 125 | 12.602 | 11.953 | 120.150 | 1.00 | 17.84 | A |
| ATOM | 835 | CG | TYR | 125 | 11.805 | 10.695 | 120.391 | 1.00 | 17.89 | A |
| ATOM | 836 | CD1 | TYR | 125 | 10.791 | 10.304 | 119.513 | 1.00 | 18.58 | A |
| ATOM | 837 | CE1 | TYR | 125 | 10.086 | 9.120 | 119.713 | 1.00 | 18.72 | A |
| ATOM | 838 | CD2 | TYR | 125 | 12.090 | 9.871 | 121.477 | 1.00 | 17.89 | A |
| ATOM | 839 | CE2 | TYR | 125 | 11.395 | 8.691 | 121.686 | 1.00 | 17.82 | A |
| ATOM | 840 | CZ | TYR | 125 | 10.398 | 8.321 | 120.804 | 1.00 | 19.43 | A |
| ATOM | 841 | OH | TYR | 125 | 9.724 | 7.142 | 121.017 | 1.00 | 23.55 | A |
| ATOM | 842 | C | TYR | 125 | 12.941 | 14.377 | 120.260 | 1.00 | 18.68 | A |
| ATOM | 843 | O | TYR | 125 | 12.678 | 15.144 | 119.338 | 1.00 | 20.06 | A |
| ATOM | 844 | N | THR | 126 | 14.061 | 14.445 | 120.971 | 1.00 | 18.30 | A |
| ATOM | 845 | CA | THR | 126 | 15.106 | 15.402 | 120.651 | 1.00 | 18.04 | A |
| ATOM | 846 | CB | THR | 126 | 16.063 | 15.618 | 121.839 | 1.00 | 18.63 | A |
| ATOM | 847 | OG1 | THR | 126 | 16.592 | 14.356 | 122.254 | 1.00 | 20.05 | A |
| ATOM | 848 | CG2 | THR | 126 | 15.339 | 16.258 | 123.014 | 1.00 | 18.83 | A |
| ATOM | 849 | C | THR | 126 | 15.838 | 14.653 | 119.537 | 1.00 | 17.89 | A |
| ATOM | 850 | O | THR | 126 | 15.606 | 13.455 | 119.355 | 1.00 | 16.79 | A |
| ATOM | 851 | N | TRP | 127 | 16.708 | 15.322 | 118.789 | 1.00 | 16.50 | A |
| ATOM | 852 | CA | TRP | 127 | 17.401 | 14.636 | 117.711 | 1.00 | 16.42 | A |
| ATOM | 853 | CB | TRP | 127 | 18.198 | 15.642 | 116.868 | 1.00 | 14.53 | A |
| ATOM | 854 | CG | TRP | 127 | 19.443 | 16.133 | 117.506 | 1.00 | 12.21 | A |
| ATOM | 855 | CD2 | TRP | 127 | 20.746 | 15.554 | 117.381 | 1.00 | 12.40 | A |
| ATOM | 856 | CE2 | TRP | 127 | 21.634 | 16.350 | 118.138 | 1.00 | 12.89 | A |
| ATOM | 857 | CE3 | TRP | 127 | 21.250 | 14.436 | 116.703 | 1.00 | 10.82 | A |
| ATOM | 858 | CD1 | TRP | 127 | 19.580 | 17.225 | 118.314 | 1.00 | 12.48 | A |
| ATOM | 859 | NE1 | TRP | 127 | 20.899 | 17.365 | 118.698 | 1.00 | 14.38 | A |
| ATOM | 860 | CZ2 | TRP | 127 | 22.997 | 16.063 | 118.233 | 1.00 | 12.67 | A |
| ATOM | 861 | CZ3 | TRP | 127 | 22.607 | 14.148 | 116.800 | 1.00 | 8.68 | A |
| ATOM | 862 | CH2 | TRP | 127 | 23.463 | 14.959 | 117.558 | 1.00 | 10.75 | A |
| ATOM | 863 | C | TRP | 127 | 18.318 | 13.500 | 118.191 | 1.00 | 18.04 | A |
| ATOM | 864 | O | TRP | 127 | 18.496 | 12.507 | 117.491 | 1.00 | 17.73 | A |
| ATOM | 865 | N | GLU | 128 | 18.874 | 13.639 | 119.390 | 1.00 | 20.55 | A |
| ATOM | 866 | CA | GLU | 128 | 19.773 | 12.630 | 119.954 | 1.00 | 22.98 | A |
| ATOM | 867 | CB | GLU | 128 | 20.449 | 13.167 | 121.216 | 1.00 | 24.66 | A |
| ATOM | 868 | CG | GLU | 128 | 21.328 | 14.375 | 121.028 | 1.00 | 30.86 | A |
| ATOM | 869 | CD | GLU | 128 | 21.812 | 14.929 | 122.359 | 1.00 | 34.39 | A |
| ATOM | 870 | OE1 | GLU | 128 | 22.271 | 14.126 | 123.204 | 1.00 | 36.58 | A |
| ATOM | 871 | OE2 | GLU | 128 | 21.734 | 16.160 | 122.562 | 1.00 | 36.22 | A |
| ATOM | 872 | C | GLU | 128 | 19.092 | 11.322 | 120.336 | 1.00 | 21.59 | A |
| ATOM | 873 | O | GLU | 128 | 19.744 | 10.291 | 120.456 | 1.00 | 20.67 | A |
| ATOM | 874 | N | GLU | 129 | 17.784 | 11.362 | 120.539 | 1.00 | 22.17 | A |
| ATOM | 875 | CA | GLU | 129 | 17.073 | 10.167 | 120.974 | 1.00 | 22.68 | A |
| ATOM | 876 | CB | GLU | 129 | 16.487 | 10.426 | 122.364 | 1.00 | 23.27 | A |
| ATOM | 877 | CG | GLU | 129 | 17.550 | 10.770 | 123.392 | 1.00 | 28.13 | A |
| ATOM | 878 | CD | GLU | 129 | 16.965 | 11.157 | 124.737 | 1.00 | 32.95 | A |
| ATOM | 879 | OE1 | GLU | 129 | 17.752 | 11.323 | 125.702 | 1.00 | 33.26 | A |
| ATOM | 880 | OE2 | GLU | 129 | 15.724 | 11.301 | 124.827 | 1.00 | 31.63 | A |
| ATOM | 881 | C | GLU | 129 | 15.983 | 9.679 | 120.035 | 1.00 | 20.72 | A |
| ATOM | 882 | O | GLU | 129 | 15.273 | 8.728 | 120.343 | 1.00 | 23.09 | A |
| ATOM | 883 | N | ASP | 130 | 15.862 | 10.322 | 118.885 | 1.00 | 18.40 | A |
| ATOM | 884 | CA | ASP | 130 | 14.846 | 9.945 | 117.918 | 1.00 | 16.36 | A |
| ATOM | 885 | CB | ASP | 130 | 14.770 | 11.015 | 116.828 | 1.00 | 15.71 | A |
| ATOM | 886 | CG | ASP | 130 | 13.495 | 10.947 | 116.031 | 1.00 | 15.49 | A |
| ATOM | 887 | OD1 | ASP | 130 | 13.044 | 12.002 | 115.545 | 1.00 | 17.27 | A |
| ATOM | 888 | OD2 | ASP | 130 | 12.950 | 9.839 | 115.874 | 1.00 | 15.06 | A |
| ATOM | 889 | C | ASP | 130 | 15.168 | 8.573 | 117.326 | 1.00 | 15.41 | A |
| ATOM | 890 | O | ASP | 130 | 16.196 | 8.377 | 116.680 | 1.00 | 15.65 | A |
| ATOM | 891 | N | PRO | 131 | 14.287 | 7.597 | 117.548 | 1.00 | 14.81 | A |
| ATOM | 892 | CD | PRO | 131 | 12.980 | 7.675 | 118.222 | 1.00 | 14.52 | A |
| ATOM | 893 | CA | PRO | 131 | 14.523 | 6.255 | 117.018 | 1.00 | 15.02 | A |
| ATOM | 894 | CB | PRO | 131 | 13.348 | 5.457 | 117.579 | 1.00 | 15.21 | A |
| ATOM | 895 | CG | PRO | 131 | 12.267 | 6.478 | 117.656 | 1.00 | 16.02 | A |
| ATOM | 896 | C | PRO | 131 | 14.607 | 6.183 | 115.492 | 1.00 | 15.04 | A |
| ATOM | 897 | O | PRO | 131 | 15.103 | 5.196 | 114.943 | 1.00 | 12.71 | A |
| ATOM | 898 | N | LEU | 132 | 14.125 | 7.224 | 114.814 | 1.00 | 14.88 | A |
| ATOM | 899 | CA | LEU | 132 | 14.161 | 7.254 | 113.354 | 1.00 | 14.03 | A |
| ATOM | 900 | CB | LEU | 132 | 12.947 | 8.007 | 112.796 | 1.00 | 12.82 | A |
| ATOM | 901 | CG | LEU | 132 | 11.562 | 7.434 | 113.129 | 1.00 | 14.44 | A |
| ATOM | 902 | CD1 | LEU | 132 | 10.506 | 8.271 | 112.397 | 1.00 | 8.97 | A |
| ATOM | 903 | CD2 | LEU | 132 | 11.470 | 5.950 | 112.724 | 1.00 | 8.90 | A |
| ATOM | 904 | C | LEU | 132 | 15.446 | 7.861 | 112.786 | 1.00 | 12.21 | A |
| ATOM | 905 | O | LEU | 132 | 15.626 | 7.916 | 111.573 | 1.00 | 11.16 | A |
| ATOM | 906 | N | ALA | 133 | 16.337 | 8.321 | 113.655 | 1.00 | 11.83 | A |
| ATOM | 907 | CA | ALA | 133 | 17.604 | 8.891 | 113.186 | 1.00 | 11.94 | A |
| ATOM | 908 | CB | ALA | 133 | 18.447 | 9.345 | 114.377 | 1.00 | 7.70 | A |
| ATOM | 909 | C | ALA | 133 | 18.367 | 7.825 | 112.373 | 1.00 | 12.53 | A |
| ATOM | 910 | O | ALA | 133 | 18.308 | 6.637 | 112.693 | 1.00 | 12.95 | A |
| ATOM | 911 | N | GLY | 134 | 19.074 | 8.256 | 111.330 | 1.00 | 13.23 | A |
| ATOM | 912 | CA | GLY | 134 | 19.832 | 7.328 | 110.506 | 1.00 | 13.31 | A |
| ATOM | 913 | C | GLY | 134 | 21.314 | 7.273 | 110.858 | 1.00 | 14.51 | A |
| ATOM | 914 | O | GLY | 134 | 21.727 | 7.771 | 111.910 | 1.00 | 12.96 | A |
| ATOM | 915 | N | ILE | 135 | 22.111 | 6.685 | 109.962 | 1.00 | 13.27 | A |
| ATOM | 916 | CA | ILE | 135 | 23.547 | 6.529 | 110.158 | 1.00 | 10.64 | A |
| ATOM | 917 | CB | ILE | 135 | 24.211 | 5.825 | 108.945 | 1.00 | 12.21 | A |
| ATOM | 918 | CG2 | ILE | 135 | 25.728 | 5.725 | 109.166 | 1.00 | 9.26 | A |
| ATOM | 919 | CG1 | ILE | 135 | 23.606 | 4.433 | 108.749 | 1.00 | 9.44 | A |
| ATOM | 920 | CD1 | ILE | 135 | 24.194 | 3.659 | 107.563 | 1.00 | 7.34 | A |
| ATOM | 921 | C | ILE | 135 | 24.319 | 7.817 | 110.429 | 1.00 | 11.04 | A |
| ATOM | 922 | O | ILE | 135 | 25.101 | 7.868 | 111.370 | 1.00 | 12.98 | A |
| ATOM | 923 | N | ILE | 136 | 24.117 | 8.843 | 109.606 | 1.00 | 10.10 | A |
| ATOM | 924 | CA | ILE | 136 | 24.822 | 10.109 | 109.783 | 1.00 | 10.16 | A |
| ATOM | 925 | CB | ILE | 136 | 24.393 | 11.137 | 108.709 | 1.00 | 9.76 | A |
| ATOM | 926 | CG2 | ILE | 136 | 25.052 | 12.489 | 108.966 | 1.00 | 7.05 | A |
| ATOM | 927 | CG1 | ILE | 136 | 24.783 | 10.611 | 107.327 | 1.00 | 8.04 | A |
| ATOM | 928 | CD1 | ILE | 136 | 24.420 | 11.555 | 106.177 | 1.00 | 8.70 | A |
| ATOM | 929 | C | ILE | 136 | 24.680 | 10.734 | 111.180 | 1.00 | 10.98 | A |
| ATOM | 930 | O | ILE | 136 | 25.673 | 10.974 | 111.848 | 1.00 | 11.07 | A |
| ATOM | 931 | N | PRO | 137 | 23.449 | 11.015 | 111.637 | 1.00 | 12.76 | A |
| ATOM | 932 | CD | PRO | 137 | 22.118 | 10.891 | 111.018 | 1.00 | 12.91 | A |
| ATOM | 933 | CA | PRO | 137 | 23.344 | 11.609 | 112.974 | 1.00 | 13.27 | A |
| ATOM | 934 | CB | PRO | 137 | 21.863 | 11.966 | 113.079 | 1.00 | 12.28 | A |
| ATOM | 935 | CG | PRO | 137 | 21.210 | 10.920 | 112.226 | 1.00 | 12.44 | A |
| ATOM | 936 | C | PRO | 137 | 23.814 | 10.707 | 114.117 | 1.00 | 13.75 | A |
| ATOM | 937 | O | PRO | 137 | 24.349 | 11.191 | 115.118 | 1.00 | 13.93 | A |
| ATOM | 938 | N | ARG | 138 | 23.616 | 9.401 | 113.982 | 1.00 | 13.99 | A |
| ATOM | 939 | CA | ARG | 138 | 24.061 | 8.490 | 115.034 | 1.00 | 14.63 | A |
| ATOM | 940 | CB | ARG | 138 | 23.520 | 7.083 | 114.788 | 1.00 | 11.07 | A |
| ATOM | 941 | CG | ARG | 138 | 22.026 | 6.971 | 115.030 | 1.00 | 10.07 | A |
| ATOM | 942 | CD | ARG | 138 | 21.514 | 5.574 | 114.706 | 1.00 | 12.89 | A |
| ATOM | 943 | NE | ARG | 138 | 20.063 | 5.502 | 114.816 | 1.00 | 14.12 | A |
| ATOM | 944 | CZ | ARG | 138 | 19.395 | 5.417 | 115.961 | 1.00 | 16.84 | A |
| ATOM | 945 | NH1 | ARG | 138 | 20.043 | 5.380 | 117.123 | 1.00 | 17.01 | A |
| ATOM | 946 | NH2 | ARG | 138 | 18.070 | 5.405 | 115.943 | 1.00 | 16.58 | A |
| ATOM | 947 | C | ARG | 138 | 25.590 | 8.479 | 115.105 | 1.00 | 14.82 | A |
| ATOM | 948 | O | ARG | 138 | 26.175 | 8.491 | 116.189 | 1.00 | 17.18 | A |
| ATOM | 949 | N | THR | 139 | 26.227 | 8.490 | 113.943 | 1.00 | 13.19 | A |
| ATOM | 950 | CA | THR | 139 | 27.676 | 8.487 | 113.864 | 1.00 | 14.27 | A |
| ATOM | 951 | CB | THR | 139 | 28.134 | 8.347 | 112.394 | 1.00 | 15.10 | A |
| ATOM | 952 | OG1 | THR | 139 | 27.671 | 7.092 | 111.877 | 1.00 | 16.74 | A |
| ATOM | 953 | CG2 | THR | 139 | 29.663 | 8.403 | 112.290 | 1.00 | 15.25 | A |
| ATOM | 954 | C | THR | 139 | 28.315 | 9.738 | 114.473 | 1.00 | 14.96 | A |
| ATOM | 955 | O | THR | 139 | 29.268 | 9.642 | 115.247 | 1.00 | 16.32 | A |
| ATOM | 956 | N | LEU | 140 | 27.802 | 10.912 | 114.128 | 1.00 | 13.16 | A |
| ATOM | 957 | CA | LEU | 140 | 28.374 | 12.136 | 114.664 | 1.00 | 13.55 | A |
| ATOM | 958 | CB | LEU | 140 | 27.742 | 13.351 | 113.988 | 1.00 | 13.68 | A |
| ATOM | 959 | CG | LEU | 140 | 28.065 | 13.435 | 112.489 | 1.00 | 15.01 | A |
| ATOM | 960 | CD1 | LEU | 140 | 27.116 | 14.410 | 111.824 | 1.00 | 15.28 | A |
| ATOM | 961 | CD2 | LEU | 140 | 29.535 | 13.845 | 112.286 | 1.00 | 12.18 | A |
| ATOM | 962 | C | LEU | 140 | 28.168 | 12.200 | 116.165 | 1.00 | 14.55 | A |
| ATOM | 963 | O | LEU | 140 | 29.031 | 12.674 | 116.900 | 1.00 | 14.87 | A |
| ATOM | 964 | N | HIS | 141 | 27.021 | 11.712 | 116.621 | 1.00 | 15.53 | A |
| ATOM | 965 | CA | HIS | 141 | 26.715 | 11.731 | 118.041 | 1.00 | 15.51 | A |
| ATOM | 966 | CB | HIS | 141 | 25.241 | 11.359 | 118.265 | 1.00 | 17.50 | A |
| ATOM | 967 | CG | HIS | 141 | 24.809 | 11.401 | 119.698 | 1.00 | 19.49 | A |
| ATOM | 968 | CD2 | HIS | 141 | 24.144 | 12.349 | 120.400 | 1.00 | 20.09 | A |
| ATOM | 969 | ND1 | HIS | 141 | 25.057 | 10.373 | 120.584 | 1.00 | 22.94 | A |
| ATOM | 970 | CE1 | HIS | 141 | 24.561 | 10.686 | 121.769 | 1.00 | 21.94 | A |
| ATOM | 971 | NE2 | HIS | 141 | 24.002 | 11.880 | 121.683 | 1.00 | 21.59 | A |
| ATOM | 972 | C | HIS | 141 | 27.638 | 10.772 | 118.787 | 1.00 | 14.45 | A |
| ATOM | 973 | O | HIS | 141 | 28.133 | 11.094 | 119.864 | 1.00 | 12.82 | A |
| ATOM | 974 | N | GLN | 142 | 27.893 | 9.606 | 118.202 | 1.00 | 12.87 | A |
| ATOM | 975 | CA | GLN | 142 | 28.753 | 8.627 | 118.852 | 1.00 | 14.02 | A |
| ATOM | 976 | CB | GLN | 142 | 28.542 | 7.248 | 118.239 | 1.00 | 13.39 | A |
| ATOM | 977 | CG | GLN | 142 | 27.299 | 6.545 | 118.741 | 1.00 | 20.05 | A |
| ATOM | 978 | CD | GLN | 142 | 27.237 | 6.484 | 120.262 | 1.00 | 21.32 | A |
| ATOM | 979 | OE1 | GLN | 142 | 26.660 | 7.361 | 120.910 | 1.00 | 21.37 | A |
| ATOM | 980 | NE2 | GLN | 142 | 27.850 | 5.454 | 120.837 | 1.00 | 19.74 | A |
| ATOM | 981 | C | GLN | 142 | 30.243 | 8.963 | 118.862 | 1.00 | 13.74 | A |
| ATOM | 982 | O | GLN | 142 | 30.961 | 8.535 | 119.759 | 1.00 | 14.17 | A |
| ATOM | 983 | N | ILE | 143 | 30.713 | 9.709 | 117.870 | 1.00 | 13.21 | A |
| ATOM | 984 | CA | ILE | 143 | 32.119 | 10.087 | 117.826 | 1.00 | 13.39 | A |
| ATOM | 985 | CB | ILE | 143 | 32.435 | 10.932 | 116.576 | 1.00 | 11.43 | A |
| ATOM | 986 | CG2 | ILE | 143 | 33.847 | 11.507 | 116.678 | 1.00 | 13.15 | A |
| ATOM | 987 | CG1 | ILE | 143 | 32.282 | 10.068 | 115.324 | 1.00 | 9.90 | A |
| ATOM | 988 | CD1 | ILE | 143 | 32.437 | 10.844 | 114.012 | 1.00 | 8.46 | A |
| ATOM | 989 | C | ILE | 143 | 32.454 | 10.897 | 119.082 | 1.00 | 14.99 | A |
| ATOM | 990 | O | ILE | 143 | 33.473 | 10.660 | 119.724 | 1.00 | 13.04 | A |
| ATOM | 991 | N | PHE | 144 | 31.581 | 11.848 | 119.419 | 1.00 | 17.68 | A |
| ATOM | 992 | CA | PHE | 144 | 31.741 | 12.694 | 120.599 | 1.00 | 20.78 | A |
| ATOM | 993 | CB | PHE | 144 | 30.771 | 13.882 | 120.548 | 1.00 | 17.56 | A |
| ATOM | 994 | CG | PHE | 144 | 31.153 | 14.924 | 119.549 | 1.00 | 18.09 | A |
| ATOM | 995 | CD1 | PHE | 144 | 32.205 | 15.796 | 119.809 | 1.00 | 18.10 | A |
| ATOM | 996 | CD2 | PHE | 144 | 30.492 | 15.013 | 118.327 | 1.00 | 17.52 | A |
| ATOM | 997 | CE1 | PHE | 144 | 32.596 | 16.740 | 118.864 | 1.00 | 19.03 | A |
| ATOM | 998 | CE2 | PHE | 144 | 30.873 | 15.949 | 117.371 | 1.00 | 16.50 | A |
| ATOM | 999 | CZ | PHE | 144 | 31.926 | 16.817 | 117.639 | 1.00 | 18.32 | A |
| ATOM | 1000 | C | PHE | 144 | 31.481 | 11.908 | 121.877 | 1.00 | 24.06 | A |
| ATOM | 1001 | O | PHE | 144 | 32.059 | 12.203 | 122.917 | 1.00 | 25.61 | A |
| ATOM | 1002 | N | GLU | 145 | 30.596 | 10.924 | 121.801 | 1.00 | 28.05 | A |
| ATOM | 1003 | CA | GLU | 145 | 30.270 | 10.113 | 122.963 | 1.00 | 32.18 | A |
| ATOM | 1004 | CB | GLU | 145 | 29.052 | 9.233 | 122.660 | 1.00 | 34.92 | A |
| ATOM | 1005 | CG | GLU | 145 | 28.382 | 8.616 | 123.877 | 1.00 | 41.48 | A |
| ATOM | 1006 | CD | GLU | 145 | 27.459 | 9.586 | 124.604 | 1.00 | 46.68 | A |
| ATOM | 1007 | OE1 | GLU | 145 | 26.808 | 9.154 | 125.583 | 1.00 | 48.85 | A |
| ATOM | 1008 | OE2 | GLU | 145 | 27.379 | 10.772 | 124.205 | 1.00 | 48.27 | A |
| ATOM | 1009 | C | GLU | 145 | 31.472 | 9.234 | 123.300 | 1.00 | 33.53 | A |
| ATOM | 1010 | O | GLU | 145 | 31.796 | 9.031 | 124.465 | 1.00 | 35.14 | A |
| ATOM | 1011 | N | LYS | 146 | 32.139 | 8.727 | 122.272 | 1.00 | 33.94 | A |
| ATOM | 1012 | CA | LYS | 146 | 33.289 | 7.857 | 122.460 | 1.00 | 35.62 | A |
| ATOM | 1013 | CB | LYS | 146 | 33.493 | 6.982 | 121.218 | 1.00 | 35.76 | A |
| ATOM | 1014 | CG | LYS | 146 | 32.398 | 5.949 | 120.990 | 1.00 | 38.40 | A |
| ATOM | 1015 | CD | LYS | 146 | 32.750 | 5.000 | 119.853 | 1.00 | 39.00 | A |
| ATOM | 1016 | CE | LYS | 146 | 31.822 | 3.804 | 119.842 | 1.00 | 40.55 | A |
| ATOM | 1017 | NZ | LYS | 146 | 32.108 | 2.871 | 118.719 | 1.00 | 42.99 | A |
| ATOM | 1018 | C | LYS | 146 | 34.600 | 8.572 | 122.781 | 1.00 | 37.30 | A |
| ATOM | 1019 | O | LYS | 146 | 35.279 | 8.224 | 123.746 | 1.00 | 38.30 | A |
| ATOM | 1020 | N | LEU | 147 | 34.959 | 9.567 | 121.978 | 1.00 | 37.75 | A |
| ATOM | 1021 | CA | LEU | 147 | 36.212 | 10.286 | 122.182 | 1.00 | 39.45 | A |
| ATOM | 1022 | CB | LEU | 147 | 36.611 | 11.013 | 120.894 | 1.00 | 36.70 | A |
| ATOM | 1023 | CG | LEU | 147 | 36.769 | 10.134 | 119.652 | 1.00 | 34.99 | A |
| ATOM | 1024 | CD1 | LEU | 147 | 37.244 | 10.979 | 118.483 | 1.00 | 32.76 | A |
| ATOM | 1025 | CD2 | LEU | 147 | 37.754 | 9.012 | 119.940 | 1.00 | 33.24 | A |
| ATOM | 1026 | C | LEU | 147 | 36.250 | 11.268 | 123.355 | 1.00 | 41.40 | A |
| ATOM | 1027 | O | LEU | 147 | 37.329 | 11.653 | 123.803 | 1.00 | 41.57 | A |
| ATOM | 1028 | N | THR | 148 | 35.091 | 11.681 | 123.855 | 1.00 | 43.50 | A |
| ATOM | 1029 | CA | THR | 148 | 35.078 | 12.613 | 124.972 | 1.00 | 46.76 | A |
| ATOM | 1030 | CB | THR | 148 | 33.735 | 13.379 | 125.068 | 1.00 | 46.73 | A |
| ATOM | 1031 | OG1 | THR | 148 | 33.559 | 14.194 | 123.901 | 1.00 | 45.09 | A |
| ATOM | 1032 | CG2 | THR | 148 | 33.717 | 14.274 | 126.299 | 1.00 | 45.59 | A |
| ATOM | 1033 | C | THR | 148 | 35.327 | 11.848 | 126.266 | 1.00 | 50.09 | A |
| ATOM | 1034 | O | THR | 148 | 36.050 | 12.321 | 127.149 | 1.00 | 50.49 | A |
| ATOM | 1035 | N | ASP | 149 | 34.734 | 10.660 | 126.367 | 1.00 | 53.41 | A |
| ATOM | 1036 | CA | ASP | 149 | 34.899 | 9.812 | 127.545 | 1.00 | 56.45 | A |
| ATOM | 1037 | CB | ASP | 149 | 34.094 | 8.515 | 127.395 | 1.00 | 57.31 | A |
| ATOM | 1038 | CG | ASP | 149 | 32.677 | 8.641 | 127.926 | 1.00 | 59.22 | A |
| ATOM | 1039 | OD1 | ASP | 149 | 32.519 | 9.073 | 129.090 | 1.00 | 59.37 | A |
| ATOM | 1040 | OD2 | ASP | 149 | 31.723 | 8.302 | 127.191 | 1.00 | 59.44 | A |
| ATOM | 1041 | C | ASP | 149 | 36.365 | 9.468 | 127.778 | 1.00 | 57.60 | A |
| ATOM | 1042 | O | ASP | 149 | 36.948 | 9.837 | 128.800 | 1.00 | 57.84 | A |
| ATOM | 1043 | N | ASN | 150 | 36.955 | 8.756 | 126.824 | 1.00 | 58.66 | A |
| ATOM | 1044 | CA | ASN | 150 | 38.354 | 8.366 | 126.919 | 1.00 | 59.63 | A |
| ATOM | 1045 | CB | ASN | 150 | 38.699 | 7.388 | 125.793 | 1.00 | 62.63 | A |
| ATOM | 1046 | CG | ASN | 150 | 37.845 | 6.129 | 125.832 | 1.00 | 65.36 | A |
| ATOM | 1047 | OD1 | ASN | 150 | 37.880 | 5.366 | 126.803 | 1.00 | 66.45 | A |
| ATOM | 1048 | ND2 | ASN | 150 | 37.070 | 5.908 | 124.774 | 1.00 | 66.13 | A |
| ATOM | 1049 | C | ASN | 150 | 39.248 | 9.598 | 126.833 | 1.00 | 58.25 | A |
| ATOM | 1050 | O | ASN | 150 | 38.814 | 10.657 | 126.382 | 1.00 | 58.50 | A |
| ATOM | 1051 | N | GLY | 151 | 40.492 | 9.459 | 127.279 | 1.00 | 56.63 | A |
| ATOM | 1052 | CA | GLY | 151 | 41.416 | 10.579 | 127.233 | 1.00 | 55.03 | A |
| ATOM | 1053 | C | GLY | 151 | 41.915 | 10.801 | 125.820 | 1.00 | 53.26 | A |
| ATOM | 1054 | O | GLY | 151 | 42.983 | 10.307 | 125.449 | 1.00 | 52.83 | A |
| ATOM | 1055 | N | THR | 152 | 41.149 | 11.551 | 125.029 | 1.00 | 50.83 | A |
| ATOM | 1056 | CA | THR | 152 | 41.519 | 11.806 | 123.643 | 1.00 | 47.73 | A |
| ATOM | 1057 | CB | THR | 152 | 40.763 | 10.858 | 122.680 | 1.00 | 47.39 | A |
| ATOM | 1058 | OG1 | THR | 152 | 40.890 | 9.502 | 123.127 | 1.00 | 48.20 | A |
| ATOM | 1059 | CG2 | THR | 152 | 41.326 | 10.975 | 121.271 | 1.00 | 45.61 | A |
| ATOM | 1060 | C | THR | 152 | 41.237 | 13.230 | 123.180 | 1.00 | 46.24 | A |
| ATOM | 1061 | O | THR | 152 | 40.163 | 13.775 | 123.425 | 1.00 | 46.24 | A |
| ATOM | 1062 | N | GLU | 153 | 42.217 | 13.828 | 122.510 | 1.00 | 43.69 | A |
| ATOM | 1063 | CA | GLU | 153 | 42.066 | 15.165 | 121.957 | 1.00 | 41.25 | A |
| ATOM | 1064 | CB | GLU | 153 | 43.386 | 15.926 | 122.014 | 1.00 | 42.93 | A |
| ATOM | 1065 | CG | GLU | 153 | 43.815 | 16.330 | 123.407 | 1.00 | 46.50 | A |
| ATOM | 1066 | CD | GLU | 153 | 45.193 | 16.952 | 123.421 | 1.00 | 48.91 | A |
| ATOM | 1067 | OE1 | GLU | 153 | 46.181 | 16.219 | 123.196 | 1.00 | 49.46 | A |
| ATOM | 1068 | OE2 | GLU | 153 | 45.288 | 18.177 | 123.649 | 1.00 | 52.22 | A |
| ATOM | 1069 | C | GLU | 153 | 41.677 | 14.898 | 120.508 | 1.00 | 38.96 | A |
| ATOM | 1070 | O | GLU | 153 | 42.232 | 13.998 | 119.874 | 1.00 | 38.36 | A |
| ATOM | 1071 | N | PHE | 154 | 40.730 | 15.665 | 119.980 | 1.00 | 35.01 | A |
| ATOM | 1072 | CA | PHE | 154 | 40.289 | 15.434 | 118.611 | 1.00 | 30.73 | A |
| ATOM | 1073 | CB | PHE | 154 | 39.416 | 14.177 | 118.574 | 1.00 | 27.60 | A |
| ATOM | 1074 | CG | PHE | 154 | 38.102 | 14.340 | 119.282 | 1.00 | 24.32 | A |
| ATOM | 1075 | CD1 | PHE | 154 | 36.965 | 14.742 | 118.585 | 1.00 | 22.22 | A |
| ATOM | 1076 | CD2 | PHE | 154 | 38.009 | 14.130 | 120.652 | 1.00 | 24.15 | A |
| ATOM | 1077 | CE1 | PHE | 154 | 35.751 | 14.929 | 119.246 | 1.00 | 22.43 | A |
| ATOM | 1078 | CE2 | PHE | 154 | 36.797 | 14.316 | 121.327 | 1.00 | 24.33 | A |
| ATOM | 1079 | CZ | PHE | 154 | 35.664 | 14.718 | 120.618 | 1.00 | 23.63 | A |
| ATOM | 1080 | C | PHE | 154 | 39.498 | 16.590 | 118.024 | 1.00 | 28.48 | A |
| ATOM | 1081 | O | PHE | 154 | 38.921 | 17.402 | 118.744 | 1.00 | 27.87 | A |
| ATOM | 1082 | N | SER | 155 | 39.474 | 16.653 | 116.702 | 1.00 | 26.86 | A |
| ATOM | 1083 | CA | SER | 155 | 38.713 | 17.676 | 116.006 | 1.00 | 25.68 | A |
| ATOM | 1084 | CB | SER | 155 | 39.635 | 18.708 | 115.347 | 1.00 | 24.22 | A |
| ATOM | 1085 | OG | SER | 155 | 40.401 | 18.131 | 114.309 | 1.00 | 25.09 | A |
| ATOM | 1086 | C | SER | 155 | 37.920 | 16.925 | 114.947 | 1.00 | 26.10 | A |
| ATOM | 1087 | O | SER | 155 | 38.402 | 15.937 | 114.380 | 1.00 | 26.26 | A |
| ATOM | 1088 | N | VAL | 156 | 36.697 | 17.377 | 114.700 | 1.00 | 25.35 | A |
| ATOM | 1089 | CA | VAL | 156 | 35.836 | 16.741 | 113.712 | 1.00 | 23.66 | A |
| ATOM | 1090 | CB | VAL | 156 | 34.549 | 16.202 | 114.371 | 1.00 | 22.75 | A |
| ATOM | 1091 | CG1 | VAL | 156 | 33.671 | 15.499 | 113.331 | 1.00 | 20.72 | A |
| ATOM | 1092 | CG2 | VAL | 156 | 34.910 | 15.257 | 115.497 | 1.00 | 20.01 | A |
| ATOM | 1093 | C | VAL | 156 | 35.447 | 17.733 | 112.622 | 1.00 | 24.01 | A |
| ATOM | 1094 | O | VAL | 156 | 34.960 | 18.832 | 112.916 | 1.00 | 24.09 | A |
| ATOM | 1095 | N | LYS | 157 | 35.679 | 17.344 | 111.369 | 1.00 | 21.25 | A |
| ATOM | 1096 | CA | LYS | 157 | 35.332 | 18.172 | 110.220 | 1.00 | 20.34 | A |
| ATOM | 1097 | CB | LYS | 157 | 36.559 | 18.467 | 109.347 | 1.00 | 24.12 | A |
| ATOM | 1098 | CG | LYS | 157 | 37.755 | 19.140 | 110.028 | 1.00 | 28.05 | A |
| ATOM | 1099 | CD | LYS | 157 | 37.474 | 20.581 | 110.410 | 1.00 | 31.98 | A |
| ATOM | 1100 | CE | LYS | 157 | 38.755 | 21.314 | 110.845 | 1.00 | 35.17 | A |
| ATOM | 1101 | NZ | LYS | 157 | 39.737 | 21.545 | 109.726 | 1.00 | 35.98 | A |
| ATOM | 1102 | C | LYS | 157 | 34.333 | 17.380 | 109.382 | 1.00 | 19.05 | A |
| ATOM | 1103 | O | LYS | 157 | 34.475 | 16.166 | 109.209 | 1.00 | 18.10 | A |
| ATOM | 1104 | N | VAL | 158 | 33.315 | 18.057 | 108.865 | 1.00 | 15.97 | A |
| ATOM | 1105 | CA | VAL | 158 | 32.340 | 17.380 | 108.025 | 1.00 | 14.22 | A |
| ATOM | 1106 | CB | VAL | 158 | 30.941 | 17.281 | 108.690 | 1.00 | 12.88 | A |
| ATOM | 1107 | CG1 | VAL | 158 | 31.014 | 16.411 | 109.931 | 1.00 | 10.13 | A |
| ATOM | 1108 | CG2 | VAL | 158 | 30.419 | 18.651 | 109.031 | 1.00 | 13.23 | A |
| ATOM | 1109 | C | VAL | 158 | 32.221 | 18.106 | 106.706 | 1.00 | 13.72 | A |
| ATOM | 1110 | O | VAL | 158 | 32.469 | 19.300 | 106.610 | 1.00 | 14.66 | A |
| ATOM | 1111 | N | SER | 159 | 31.845 | 17.373 | 105.677 | 1.00 | 14.86 | A |
| ATOM | 1112 | CA | SER | 159 | 31.702 | 17.955 | 104.362 | 1.00 | 16.10 | A |
| ATOM | 1113 | CB | SER | 159 | 33.034 | 17.844 | 103.618 | 1.00 | 17.14 | A |
| ATOM | 1114 | OG | SER | 159 | 32.904 | 18.263 | 102.279 | 1.00 | 23.83 | A |
| ATOM | 1115 | C | SER | 159 | 30.609 | 17.186 | 103.642 | 1.00 | 15.89 | A |
| ATOM | 1116 | O | SER | 159 | 30.477 | 15.976 | 103.822 | 1.00 | 15.28 | A |
| ATOM | 1117 | N | LEU | 160 | 29.820 | 17.890 | 102.838 | 1.00 | 15.69 | A |
| ATOM | 1118 | CA | LEU | 160 | 28.728 | 17.268 | 102.098 | 1.00 | 15.26 | A |
| ATOM | 1119 | CB | LEU | 160 | 27.388 | 17.679 | 102.715 | 1.00 | 15.28 | A |
| ATOM | 1120 | CG | LEU | 160 | 26.121 | 17.071 | 102.104 | 1.00 | 15.37 | A |
| ATOM | 1121 | CD1 | LEU | 160 | 26.236 | 15.559 | 102.087 | 1.00 | 12.97 | A |
| ATOM | 1122 | CD2 | LEU | 160 | 24.904 | 17.517 | 102.904 | 1.00 | 14.38 | A |
| ATOM | 1123 | C | LEU | 160 | 28.799 | 17.689 | 100.640 | 1.00 | 15.74 | A |
| ATOM | 1124 | O | LEU | 160 | 28.331 | 18.766 | 100.263 | 1.00 | 15.17 | A |
| ATOM | 1125 | N | LEU | 161 | 29.394 | 16.822 | 99.829 | 1.00 | 15.44 | A |
| ATOM | 1126 | CA | LEU | 161 | 29.577 | 17.052 | 98.401 | 1.00 | 15.04 | A |
| ATOM | 1127 | CB | LEU | 161 | 30.923 | 16.472 | 97.968 | 1.00 | 16.39 | A |
| ATOM | 1128 | CG | LEU | 161 | 31.753 | 17.038 | 96.815 | 1.00 | 19.66 | A |
| ATOM | 1129 | CD1 | LEU | 161 | 32.749 | 15.955 | 96.386 | 1.00 | 20.66 | A |
| ATOM | 1130 | CD2 | LEU | 161 | 30.887 | 17.437 | 95.641 | 1.00 | 20.16 | A |
| ATOM | 1131 | C | LEU | 161 | 28.470 | 16.311 | 97.680 | 1.00 | 15.70 | A |
| ATOM | 1132 | O | LEU | 161 | 28.200 | 15.161 | 97.989 | 1.00 | 17.10 | A |
| ATOM | 1133 | N | GLU | 162 | 27.829 | 16.952 | 96.713 | 1.00 | 15.78 | A |
| ATOM | 1134 | CA | GLU | 162 | 26.763 | 16.286 | 95.984 | 1.00 | 13.96 | A |
| ATOM | 1135 | CB | GLU | 162 | 25.413 | 16.834 | 96.428 | 1.00 | 14.46 | A |
| ATOM | 1136 | CG | GLU | 162 | 25.218 | 16.645 | 97.928 | 1.00 | 17.99 | A |
| ATOM | 1137 | CD | GLU | 162 | 23.781 | 16.776 | 98.372 | 1.00 | 18.53 | A |
| ATOM | 1138 | OE1 | GLU | 162 | 23.532 | 16.663 | 99.588 | 1.00 | 20.86 | A |
| ATOM | 1139 | OE2 | GLU | 162 | 22.902 | 16.984 | 97.513 | 1.00 | 17.99 | A |
| ATOM | 1140 | C | GLU | 162 | 26.948 | 16.403 | 94.489 | 1.00 | 12.56 | A |
| ATOM | 1141 | O | GLU | 162 | 27.425 | 17.414 | 93.985 | 1.00 | 12.95 | A |
| ATOM | 1142 | N | ILE | 163 | 26.575 | 15.346 | 93.782 | 1.00 | 11.75 | A |
| ATOM | 1143 | CA | ILE | 163 | 26.736 | 15.303 | 92.340 | 1.00 | 11.19 | A |
| ATOM | 1144 | CB | ILE | 163 | 27.588 | 14.077 | 91.941 | 1.00 | 10.80 | A |
| ATOM | 1145 | CG2 | ILE | 163 | 27.790 | 14.044 | 90.436 | 1.00 | 9.29 | A |
| ATOM | 1146 | CG1 | ILE | 163 | 28.927 | 14.121 | 92.681 | 1.00 | 10.31 | A |
| ATOM | 1147 | CD1 | ILE | 163 | 29.667 | 12.777 | 92.718 | 1.00 | 12.19 | A |
| ATOM | 1148 | C | ILE | 163 | 25.393 | 15.238 | 91.626 | 1.00 | 11.81 | A |
| ATOM | 1149 | O | ILE | 163 | 24.524 | 14.441 | 91.985 | 1.00 | 13.50 | A |
| ATOM | 1150 | N | TYR | 164 | 25.228 | 16.089 | 90.620 | 1.00 | 10.80 | A |
| ATOM | 1151 | CA | TYR | 164 | 24.011 | 16.125 | 89.826 | 1.00 | 11.96 | A |
| ATOM | 1152 | CB | TYR | 164 | 23.038 | 17.194 | 90.353 | 1.00 | 11.56 | A |
| ATOM | 1153 | CG | TYR | 164 | 21.746 | 17.240 | 89.573 | 1.00 | 10.77 | A |
| ATOM | 1154 | CD1 | TYR | 164 | 21.639 | 18.005 | 88.408 | 1.00 | 9.75 | A |
| ATOM | 1155 | CE1 | TYR | 164 | 20.479 | 17.991 | 87.638 | 1.00 | 8.60 | A |
| ATOM | 1156 | CD2 | TYR | 164 | 20.653 | 16.457 | 89.954 | 1.00 | 8.92 | A |
| ATOM | 1157 | CE2 | TYR | 164 | 19.483 | 16.428 | 89.187 | 1.00 | 9.51 | A |
| ATOM | 1158 | CZ | TYR | 164 | 19.405 | 17.197 | 88.031 | 1.00 | 10.37 | A |
| ATOM | 1159 | OH | TYR | 164 | 18.264 | 17.167 | 87.261 | 1.00 | 9.00 | A |
| ATOM | 1160 | C | TYR | 164 | 24.415 | 16.443 | 88.395 | 1.00 | 12.68 | A |
| ATOM | 1161 | O | TYR | 164 | 25.048 | 17.468 | 88.131 | 1.00 | 13.49 | A |
| ATOM | 1162 | N | ASN | 165 | 24.075 | 15.550 | 87.478 | 1.00 | 12.65 | A |
| ATOM | 1163 | CA | ASN | 165 | 24.410 | 15.745 | 86.078 | 1.00 | 14.45 | A |
| ATOM | 1164 | CB | ASN | 165 | 23.541 | 16.864 | 85.515 | 1.00 | 18.24 | A |
| ATOM | 1165 | CG | ASN | 165 | 23.498 | 16.869 | 84.010 | 1.00 | 24.46 | A |
| ATOM | 1166 | OD1 | ASN | 165 | 23.396 | 15.817 | 83.374 | 1.00 | 29.01 | A |
| ATOM | 1167 | ND2 | ASN | 165 | 23.556 | 18.061 | 83.422 | 1.00 | 27.99 | A |
| ATOM | 1168 | C | ASN | 165 | 25.903 | 16.069 | 85.930 | 1.00 | 14.74 | A |
| ATOM | 1169 | O | ASN | 165 | 26.290 | 16.972 | 85.184 | 1.00 | 13.82 | A |
| ATOM | 1170 | N | GLU | 166 | 26.729 | 15.321 | 86.663 | 1.00 | 13.32 | A |
| ATOM | 1171 | CA | GLU | 166 | 28.178 | 15.475 | 86.645 | 1.00 | 13.84 | A |
| ATOM | 1172 | CB | GLU | 166 | 28.730 | 15.118 | 85.265 | 1.00 | 11.37 | A |
| ATOM | 1173 | CG | GLU | 166 | 28.676 | 13.635 | 84.952 | 1.00 | 13.48 | A |
| ATOM | 1174 | CD | GLU | 166 | 29.270 | 12.781 | 86.069 | 1.00 | 15.85 | A |
| ATOM | 1175 | OE1 | GLU | 166 | 28.518 | 12.411 | 86.995 | 1.00 | 14.50 | A |
| ATOM | 1176 | OE2 | GLU | 166 | 30.491 | 12.490 | 86.022 | 1.00 | 14.74 | A |
| ATOM | 1177 | C | GLU | 166 | 28.724 | 16.835 | 87.067 | 1.00 | 15.33 | A |
| ATOM | 1178 | O | GLU | 166 | 29.809 | 17.229 | 86.650 | 1.00 | 16.01 | A |
| ATOM | 1179 | N | GLU | 167 | 27.970 | 17.555 | 87.885 | 1.00 | 16.84 | A |
| ATOM | 1180 | CA | GLU | 167 | 28.415 | 18.850 | 88.381 | 1.00 | 16.72 | A |
| ATOM | 1181 | CB | GLU | 167 | 27.403 | 19.949 | 88.052 | 1.00 | 19.43 | A |
| ATOM | 1182 | CG | GLU | 167 | 27.235 | 20.216 | 86.570 | 1.00 | 23.50 | A |
| ATOM | 1183 | CD | GLU | 167 | 26.307 | 21.388 | 86.309 | 1.00 | 28.67 | A |
| ATOM | 1184 | OE1 | GLU | 167 | 25.176 | 21.382 | 86.846 | 1.00 | 32.20 | A |
| ATOM | 1185 | OE2 | GLU | 167 | 26.707 | 22.316 | 85.571 | 1.00 | 31.83 | A |
| ATOM | 1186 | C | GLU | 167 | 28.522 | 18.685 | 89.888 | 1.00 | 15.13 | A |
| ATOM | 1187 | O | GLU | 167 | 27.773 | 17.908 | 90.480 | 1.00 | 15.63 | A |
| ATOM | 1188 | N | LEU | 168 | 29.449 | 19.408 | 90.501 | 1.00 | 12.84 | A |
| ATOM | 1189 | CA | LEU | 168 | 29.672 | 19.312 | 91.939 | 1.00 | 12.94 | A |
| ATOM | 1190 | CB | LEU | 168 | 31.171 | 19.220 | 92.217 | 1.00 | 14.17 | A |
| ATOM | 1191 | CG | LEU | 168 | 31.859 | 17.853 | 92.232 | 1.00 | 18.45 | A |
| ATOM | 1192 | CD1 | LEU | 168 | 31.289 | 16.947 | 91.164 | 1.00 | 19.30 | A |
| ATOM | 1193 | CD2 | LEU | 168 | 33.366 | 18.058 | 92.047 | 1.00 | 18.21 | A |
| ATOM | 1194 | C | LEU | 168 | 29.080 | 20.467 | 92.732 | 1.00 | 11.51 | A |
| ATOM | 1195 | O | LEU | 168 | 29.228 | 21.631 | 92.357 | 1.00 | 12.03 | A |
| ATOM | 1196 | N | PHE | 169 | 28.415 | 20.138 | 93.834 | 1.00 | 8.76 | A |
| ATOM | 1197 | CA | PHE | 169 | 27.812 | 21.152 | 94.682 | 1.00 | 10.79 | A |
| ATOM | 1198 | CB | PHE | 169 | 26.286 | 21.155 | 94.543 | 1.00 | 8.69 | A |
| ATOM | 1199 | CG | PHE | 169 | 25.804 | 21.329 | 93.127 | 1.00 | 9.29 | A |
| ATOM | 1200 | CD1 | PHE | 169 | 25.568 | 20.219 | 92.314 | 1.00 | 8.53 | A |
| ATOM | 1201 | CD2 | PHE | 169 | 25.605 | 22.595 | 92.598 | 1.00 | 7.95 | A |
| ATOM | 1202 | CE1 | PHE | 169 | 25.140 | 20.372 | 90.996 | 1.00 | 9.35 | A |
| ATOM | 1203 | CE2 | PHE | 169 | 25.178 | 22.762 | 91.284 | 1.00 | 7.75 | A |
| ATOM | 1204 | CZ | PHE | 169 | 24.945 | 21.648 | 90.479 | 1.00 | 9.59 | A |
| ATOM | 1205 | C | PHE | 169 | 28.187 | 20.923 | 96.138 | 1.00 | 12.65 | A |
| ATOM | 1206 | O | PHE | 169 | 28.319 | 19.788 | 96.593 | 1.00 | 13.12 | A |
| ATOM | 1207 | N | ASP | 170 | 26.369 | 22.027 | 96.850 | 1.00 | 12.78 | A |
| ATOM | 1208 | CA | ASP | 170 | 28.724 | 22.018 | 98.253 | 1.00 | 13.35 | A |
| ATOM | 1209 | CB | ASP | 170 | 29.817 | 23.060 | 98.502 | 1.00 | 12.29 | A |
| ATOM | 1210 | CG | ASP | 170 | 30.300 | 23.072 | 99.931 | 1.00 | 13.08 | A |
| ATOM | 1211 | OD1 | ASP | 170 | 29.577 | 22.566 | 100.817 | 1.00 | 14.08 | A |
| ATOM | 1212 | OD2 | ASP | 170 | 31.404 | 23.598 | 100.176 | 1.00 | 15.39 | A |
| ATOM | 1213 | C | ASP | 170 | 27.456 | 22.413 | 99.001 | 1.00 | 15.21 | A |
| ATOM | 1214 | O | ASP | 170 | 27.086 | 23.588 | 99.003 | 1.00 | 13.76 | A |
| ATOM | 1215 | N | LEU | 171 | 26.797 | 21.445 | 99.635 | 1.00 | 16.64 | A |
| ATOM | 1216 | CA | LEU | 171 | 25.563 | 21.722 | 100.365 | 1.00 | 19.47 | A |
| ATOM | 1217 | CB | LEU | 171 | 24.650 | 20.483 | 100.376 | 1.00 | 18.16 | A |
| ATOM | 1218 | CG | LEU | 171 | 23.677 | 20.315 | 99.200 | 1.00 | 20.70 | A |
| ATOM | 1219 | CD1 | LEU | 171 | 22.739 | 21.515 | 99.130 | 1.00 | 21.59 | A |
| ATOM | 1220 | CD2 | LEU | 171 | 24.436 | 20.192 | 97.900 | 1.00 | 19.74 | A |
| ATOM | 1221 | C | LEU | 171 | 25.724 | 22.233 | 101.794 | 1.00 | 21.95 | A |
| ATOM | 1222 | O | LEU | 171 | 24.747 | 22.282 | 102.536 | 1.00 | 24.93 | A |
| ATOM | 1223 | N | LEU | 172 | 26.931 | 22.618 | 102.197 | 1.00 | 24.33 | A |
| ATOM | 1224 | CA | LEU | 172 | 27.108 | 23.129 | 103.558 | 1.00 | 25.95 | A |
| ATOM | 1225 | CB | LEU | 172 | 28.101 | 22.267 | 104.353 | 1.00 | 22.64 | A |
| ATOM | 1226 | CG | LEU | 172 | 27.683 | 20.835 | 104.713 | 1.00 | 21.08 | A |
| ATOM | 1227 | CD1 | LEU | 172 | 28.747 | 20.208 | 105.584 | 1.00 | 19.49 | A |
| ATOM | 1228 | CD2 | LEU | 172 | 26.353 | 20.821 | 105.450 | 1.00 | 20.02 | A |
| ATOM | 1229 | C | LEU | 172 | 27.550 | 24.592 | 103.579 | 1.00 | 28.46 | A |
| ATOM | 1230 | O | LEU | 172 | 27.222 | 25.328 | 104.512 | 1.00 | 33.47 | A |
| ATOM | 1231 | N | ASN | 173 | 28.280 | 25.020 | 102.557 | 1.00 | 27.52 | A |
| ATOM | 1232 | CA | ASN | 173 | 28.733 | 26.404 | 102.479 | 1.00 | 28.63 | A |
| ATOM | 1233 | CB | ASN | 173 | 29.491 | 26.621 | 101.166 | 1.00 | 28.72 | A |
| ATOM | 1234 | CG | ASN | 173 | 30.022 | 28.037 | 101.013 | 1.00 | 30.51 | A |
| ATOM | 1235 | OD1 | ASN | 173 | 30.709 | 28.350 | 100.038 | 1.00 | 32.23 | A |
| ATOM | 1236 | ND2 | ASN | 173 | 29.709 | 28.898 | 101.969 | 1.00 | 31.50 | A |
| ATOM | 1237 | C | ASN | 173 | 27.514 | 27.331 | 102.555 | 1.00 | 30.66 | A |
| ATOM | 1238 | O | ASN | 173 | 26.639 | 27.296 | 101.688 | 1.00 | 30.81 | A |
| ATOM | 1239 | N | PRO | 174 | 27.434 | 28.164 | 103.602 | 1.00 | 32.10 | A |
| ATOM | 1240 | CD | PRO | 174 | 28.196 | 28.086 | 104.862 | 1.00 | 32.35 | A |
| ATOM | 1241 | CA | PRO | 174 | 26.298 | 29.076 | 103.741 | 1.00 | 34.00 | A |
| ATOM | 1242 | CB | PRO | 174 | 26.085 | 29.107 | 105.243 | 1.00 | 33.56 | A |
| ATOM | 1243 | CG | PRO | 174 | 27.500 | 29.119 | 105.740 | 1.00 | 33.25 | A |
| ATOM | 1244 | C | PRO | 174 | 26.566 | 30.469 | 103.179 | 1.00 | 35.77 | A |
| ATOM | 1245 | O | PRO | 174 | 26.014 | 31.452 | 103.667 | 1.00 | 38.93 | A |
| ATOM | 1246 | N | SER | 175 | 27.404 | 30.557 | 102.155 | 1.00 | 36.48 | A |
| ATOM | 1247 | CA | SER | 175 | 27.734 | 31.848 | 101.568 | 1.00 | 36.56 | A |
| ATOM | 1248 | CB | SER | 175 | 29.104 | 32.312 | 102.064 | 1.00 | 36.53 | A |
| ATOM | 1249 | OG | SER | 175 | 29.142 | 32.334 | 103.481 | 1.00 | 38.61 | A |
| ATOM | 1250 | C | SER | 175 | 27.746 | 31.745 | 100.059 | 1.00 | 36.99 | A |
| ATOM | 1251 | O | SER | 175 | 28.234 | 32.639 | 99.366 | 1.00 | 37.49 | A |
| ATOM | 1252 | N | SER | 176 | 27.226 | 30.631 | 99.560 | 1.00 | 37.22 | A |
| ATOM | 1253 | CA | SER | 176 | 27.142 | 30.385 | 98.125 | 1.00 | 38.02 | A |
| ATOM | 1254 | CB | SER | 176 | 28.296 | 29.483 | 97.662 | 1.00 | 37.78 | A |
| ATOM | 1255 | OG | SER | 176 | 28.200 | 28.177 | 98.213 | 1.00 | 37.44 | A |
| ATOM | 1256 | C | SER | 176 | 25.807 | 29.699 | 97.862 | 1.00 | 37.53 | A |
| ATOM | 1257 | O | SER | 176 | 25.277 | 29.016 | 98.734 | 1.00 | 37.34 | A |
| ATOM | 1258 | N | ASP | 177 | 25.248 | 29.891 | 96.676 | 1.00 | 38.02 | A |
| ATOM | 1259 | CA | ASP | 177 | 23.983 | 29.243 | 96.366 | 1.00 | 39.18 | A |
| ATOM | 1260 | CB | ASP | 177 | 23.012 | 30.229 | 95.704 | 1.00 | 41.03 | A |
| ATOM | 1261 | CG | ASP | 177 | 23.585 | 30.879 | 94.466 | 1.00 | 42.23 | A |
| ATOM | 1262 | OD1 | ASP | 177 | 23.936 | 30.156 | 93.511 | 1.00 | 43.11 | A |
| ATOM | 1263 | OD2 | ASP | 177 | 23.679 | 32.122 | 94.447 | 1.00 | 44.29 | A |
| ATOM | 1264 | C | ASP | 177 | 24.219 | 28.031 | 95.471 | 1.00 | 38.57 | A |
| ATOM | 1265 | O | ASP | 177 | 25.274 | 27.910 | 94.849 | 1.00 | 37.31 | A |
| ATOM | 1266 | N | VAL | 178 | 23.232 | 27.141 | 95.415 | 1.00 | 38.30 | A |
| ATOM | 1267 | CA | VAL | 178 | 23.329 | 25.918 | 94.626 | 1.00 | 38.53 | A |
| ATOM | 1268 | CB | VAL | 178 | 22.091 | 25.018 | 94.830 | 1.00 | 38.67 | A |
| ATOM | 1269 | CG1 | VAL | 178 | 22.040 | 24.532 | 96.266 | 1.00 | 38.55 | A |
| ATOM | 1270 | CG2 | VAL | 178 | 20.828 | 25.780 | 94.472 | 1.00 | 38.63 | A |
| ATOM | 1271 | C | VAL | 178 | 23.526 | 26.111 | 93.129 | 1.00 | 38.49 | A |
| ATOM | 1272 | O | VAL | 178 | 23.589 | 25.138 | 92.385 | 1.00 | 39.24 | A |
| ATOM | 1273 | N | SER | 179 | 23.618 | 27.357 | 92.683 | 1.00 | 38.10 | A |
| ATOM | 1274 | CA | SER | 179 | 23.823 | 27.626 | 91.268 | 1.00 | 37.56 | A |
| ATOM | 1275 | CB | SER | 179 | 23.265 | 29.000 | 90.905 | 1.00 | 39.68 | A |
| ATOM | 1276 | OG | SER | 179 | 21.942 | 29.155 | 91.390 | 1.00 | 45.54 | A |
| ATOM | 1277 | C | SER | 179 | 25.318 | 27.594 | 90.981 | 1.00 | 36.56 | A |
| ATOM | 1278 | O | SER | 179 | 25.740 | 27.516 | 89.828 | 1.00 | 37.57 | A |
| ATOM | 1279 | N | GLU | 180 | 26.112 | 27.663 | 92.044 | 1.00 | 34.30 | A |
| ATOM | 1280 | CA | GLU | 180 | 27.566 | 27.651 | 91.938 | 1.00 | 34.69 | A |
| ATOM | 1281 | CB | GLU | 180 | 28.173 | 28.564 | 93.018 | 1.00 | 36.86 | A |
| ATOM | 1282 | CG | GLU | 180 | 27.906 | 30.055 | 92.767 | 1.00 | 41.33 | A |
| ATOM | 1283 | CD | GLU | 180 | 28.262 | 30.958 | 93.945 | 1.00 | 42.95 | A |
| ATOM | 1284 | OE1 | GLU | 180 | 27.629 | 30.832 | 95.017 | 1.00 | 43.98 | A |
| ATOM | 1285 | OE2 | GLU | 180 | 29.174 | 31.798 | 93.795 | 1.00 | 44.03 | A |
| ATOM | 1286 | C | GLU | 180 | 28.147 | 26.241 | 92.048 | 1.00 | 32.62 | A |
| ATOM | 1287 | O | GLU | 180 | 28.084 | 25.614 | 93.104 | 1.00 | 31.99 | A |
| ATOM | 1288 | N | ARG | 181 | 28.706 | 25.745 | 90.951 | 1.00 | 30.63 | A |
| ATOM | 1289 | CA | ARG | 181 | 29.292 | 24.415 | 90.941 | 1.00 | 30.51 | A |
| ATOM | 1290 | CB | ARG | 181 | 29.050 | 23.739 | 89.587 | 1.00 | 34.25 | A |
| ATOM | 1291 | CG | ARG | 181 | 29.575 | 24.493 | 88.379 | 1.00 | 40.52 | A |
| ATOM | 1292 | CD | ARG | 181 | 29.025 | 23.901 | 87.069 | 1.00 | 46.73 | A |
| ATOM | 1293 | NE | ARG | 181 | 29.587 | 22.592 | 86.721 | 1.00 | 50.11 | A |
| ATOM | 1294 | CZ | ARG | 181 | 30.818 | 22.400 | 86.251 | 1.00 | 52.44 | A |
| ATOM | 1295 | NH1 | ARG | 181 | 31.629 | 23.435 | 86.070 | 1.00 | 53.59 | A |
| ATOM | 1296 | NH2 | ARG | 181 | 31.236 | 21.173 | 85.951 | 1.00 | 52.52 | A |
| ATOM | 1297 | C | ARG | 181 | 30.781 | 24.480 | 91.249 | 1.00 | 28.82 | A |
| ATOM | 1298 | O | ARG | 181 | 31.438 | 25.483 | 90.979 | 1.00 | 29.29 | A |
| ATOM | 1299 | N | LEU | 182 | 31.308 | 23.408 | 91.829 | 1.00 | 25.57 | A |
| ATOM | 1300 | CA | LEU | 182 | 32.718 | 23.348 | 92.182 | 1.00 | 21.92 | A |
| ATOM | 1301 | CB | LEU | 182 | 32.899 | 22.553 | 93.471 | 1.00 | 20.02 | A |
| ATOM | 1302 | CG | LEU | 182 | 32.155 | 23.087 | 94.700 | 1.00 | 20.20 | A |
| ATOM | 1303 | CD1 | LEU | 182 | 32.161 | 22.044 | 95.812 | 1.00 | 17.99 | A |
| ATOM | 1304 | CD2 | LEU | 182 | 32.802 | 24.379 | 95.159 | 1.00 | 16.82 | A |
| ATOM | 1305 | C | LEU | 182 | 33.515 | 22.696 | 91.069 | 1.00 | 22.08 | A |
| ATOM | 1306 | O | LEU | 182 | 32.960 | 21.949 | 90.257 | 1.00 | 19.82 | A |
| ATOM | 1307 | N | GLN | 183 | 34.814 | 23.000 | 91.028 | 1.00 | 22.61 | A |
| ATOM | 1308 | CA | GLN | 183 | 35.726 | 22.435 | 90.034 | 1.00 | 20.55 | A |
| ATOM | 1309 | CB | GLN | 183 | 36.702 | 23.488 | 89.523 | 1.00 | 22.39 | A |
| ATOM | 1310 | CG | GLN | 183 | 36.100 | 24.557 | 88.652 | 1.00 | 28.44 | A |
| ATOM | 1311 | CD | GLN | 183 | 36.981 | 25.799 | 88.593 | 1.00 | 32.88 | A |
| ATOM | 1312 | OE1 | GLN | 183 | 37.054 | 26.572 | 89.557 | 1.00 | 34.28 | A |
| ATOM | 1313 | NE2 | GLN | 183 | 37.664 | 25.989 | 87.468 | 1.00 | 33.10 | A |
| ATOM | 1314 | C | GLN | 183 | 36.518 | 21.327 | 90.702 | 1.00 | 19.22 | A |
| ATOM | 1315 | O | GLN | 183 | 36.795 | 21.390 | 91.897 | 1.00 | 18.40 | A |
| ATOM | 1316 | N | MET | 184 | 36.902 | 20.330 | 89.915 | 1.00 | 18.69 | A |
| ATOM | 1317 | CA | MET | 184 | 37.646 | 19.191 | 90.416 | 1.00 | 19.64 | A |
| ATOM | 1318 | CB | MET | 184 | 36.747 | 17.951 | 90.361 | 1.00 | 21.90 | A |
| ATOM | 1319 | CG | MET | 184 | 37.304 | 16.701 | 91.011 | 1.00 | 25.13 | A |
| ATOM | 1320 | SD | MET | 184 | 36.147 | 15.306 | 90.921 | 1.00 | 31.12 | A |
| ATOM | 1321 | CE | MET | 184 | 36.591 | 14.620 | 89.352 | 1.00 | 23.65 | A |
| ATOM | 1322 | C | MET | 184 | 38.897 | 18.983 | 89.568 | 1.00 | 21.60 | A |
| ATOM | 1323 | O | MET | 184 | 38.840 | 19.035 | 88.341 | 1.00 | 21.33 | A |
| ATOM | 1324 | N | PHE | 185 | 40.026 | 18.750 | 90.230 | 1.00 | 23.48 | A |
| ATOM | 1325 | CA | PHE | 185 | 41.299 | 18.531 | 89.544 | 1.00 | 25.16 | A |
| ATOM | 1326 | CB | PHE | 185 | 42.231 | 19.736 | 89.709 | 1.00 | 25.59 | A |
| ATOM | 1327 | CG | PHE | 185 | 41.595 | 21.064 | 89.414 | 1.00 | 25.42 | A |
| ATOM | 1328 | CD1 | PHE | 185 | 40.791 | 21.691 | 90.360 | 1.00 | 23.63 | A |
| ATOM | 1329 | CD2 | PHE | 185 | 41.857 | 21.718 | 88.211 | 1.00 | 26.39 | A |
| ATOM | 1330 | CE1 | PHE | 185 | 40.261 | 22.956 | 90.124 | 1.00 | 24.23 | A |
| ATOM | 1331 | CE2 | PHE | 185 | 41.332 | 22.987 | 87.961 | 1.00 | 27.17 | A |
| ATOM | 1332 | CZ | PHE | 185 | 40.533 | 23.609 | 88.921 | 1.00 | 25.70 | A |
| ATOM | 1333 | C | PHE | 185 | 42.002 | 17.326 | 90.149 | 1.00 | 26.03 | A |
| ATOM | 1334 | O | PHE | 185 | 41.709 | 16.937 | 91.275 | 1.00 | 25.54 | A |
| ATOM | 1335 | N | ASP | 186 | 42.941 | 16.743 | 89.414 | 1.00 | 29.33 | A |
| ATOM | 1336 | CA | ASP | 186 | 43.692 | 15.603 | 89.930 | 1.00 | 33.38 | A |
| ATOM | 1337 | CB | ASP | 186 | 44.461 | 14.913 | 88.801 | 1.00 | 35.26 | A |
| ATOM | 1338 | CG | ASP | 186 | 43.546 | 14.212 | 87.816 | 1.00 | 37.12 | A |
| ATOM | 1339 | OD1 | ASP | 186 | 43.644 | 14.505 | 86.603 | 1.00 | 37.66 | A |
| ATOM | 1340 | OD2 | ASP | 186 | 42.733 | 13.368 | 88.257 | 1.00 | 36.31 | A |
| ATOM | 1341 | C | ASP | 186 | 44.675 | 16.117 | 90.977 | 1.00 | 35.30 | A |
| ATOM | 1342 | O | ASP | 186 | 45.167 | 17.238 | 90.865 | 1.00 | 35.53 | A |
| ATOM | 1343 | N | ASP | 187 | 44.959 | 15.313 | 91.996 | 1.00 | 38.26 | A |
| ATOM | 1344 | CA | ASP | 187 | 45.890 | 15.739 | 93.037 | 1.00 | 43.31 | A |
| ATOM | 1345 | CB | ASP | 187 | 45.489 | 15.138 | 94.385 | 1.00 | 42.12 | A |
| ATOM | 1346 | CG | ASP | 187 | 46.217 | 15.784 | 95.546 | 1.00 | 42.51 | A |
| ATOM | 1347 | OD1 | ASP | 187 | 45.755 | 15.631 | 96.696 | 1.00 | 42.87 | A |
| ATOM | 1348 | OD2 | ASP | 187 | 47.252 | 16.442 | 95.307 | 1.00 | 41.23 | A |
| ATOM | 1349 | C | ASP | 187 | 47.307 | 15.318 | 92.665 | 1.00 | 46.67 | A |
| ATOM | 1350 | O | ASP | 187 | 47.644 | 14.138 | 92.719 | 1.00 | 48.15 | A |
| ATOM | 1351 | N | PRO | 188 | 48.160 | 16.283 | 92.286 | 1.00 | 50.27 | A |
| ATOM | 1352 | CD | PRO | 188 | 47.945 | 17.735 | 92.408 | 1.00 | 50.91 | A |
| ATOM | 1353 | CA | PRO | 188 | 49.548 | 15.996 | 91.897 | 1.00 | 53.10 | A |
| ATOM | 1354 | CB | PRO | 188 | 50.107 | 17.376 | 91.561 | 1.00 | 52.20 | A |
| ATOM | 1355 | CG | PRO | 188 | 49.364 | 18.263 | 92.503 | 1.00 | 52.65 | A |
| ATOM | 1356 | C | PRO | 188 | 50.366 | 15.279 | 92.966 | 1.00 | 55.80 | A |
| ATOM | 1357 | O | PRO | 188 | 51.319 | 14.568 | 92.650 | 1.00 | 56.91 | A |
| ATOM | 1358 | N | ARG | 189 | 49.996 | 15.466 | 94.228 | 1.00 | 58.59 | A |
| ATOM | 1359 | CA | ARG | 189 | 50.703 | 14.812 | 95.321 | 1.00 | 61.67 | A |
| ATOM | 1360 | CB | ARG | 189 | 50.294 | 15.428 | 96.658 | 1.00 | 63.13 | A |
| ATOM | 1361 | CG | ARG | 189 | 50.839 | 16.823 | 96.881 | 1.00 | 65.91 | A |
| ATOM | 1362 | CD | ARG | 189 | 50.181 | 17.468 | 98.083 | 1.00 | 68.55 | A |
| ATOM | 1363 | NE | ARG | 189 | 48.754 | 17.670 | 97.855 | 1.00 | 70.63 | A |
| ATOM | 1364 | CZ | ARG | 189 | 47.906 | 18.095 | 98.784 | 1.00 | 72.05 | A |
| ATOM | 1365 | NH1 | ARG | 189 | 48.340 | 18.362 | 100.010 | 1.00 | 72.50 | A |
| ATOM | 1366 | NH2 | ARG | 189 | 46.623 | 18.252 | 98.484 | 1.00 | 72.44 | A |
| ATOM | 1367 | C | ARG | 189 | 50.402 | 13.316 | 95.321 | 1.00 | 63.14 | A |
| ATOM | 1368 | O | ARG | 189 | 51.085 | 12.537 | 94.652 | 1.00 | 63.21 | A |
| ATOM | 1369 | N | ASN | 190 | 49.377 | 12.916 | 96.070 | 1.00 | 64.30 | A |
| ATOM | 1370 | CA | ASN | 190 | 49.000 | 11.509 | 96.140 | 1.00 | 65.20 | A |
| ATOM | 1371 | CB | ASN | 190 | 48.225 | 11.220 | 97.439 | 1.00 | 66.56 | A |
| ATOM | 1372 | CG | ASN | 190 | 47.172 | 12.273 | 97.753 | 1.00 | 67.73 | A |
| ATOM | 1373 | OD1 | ASN | 190 | 47.491 | 13.443 | 97.982 | 1.00 | 67.83 | A |
| ATOM | 1374 | ND2 | ASN | 190 | 45.909 | 11.858 | 97.773 | 1.00 | 67.20 | A |
| ATOM | 1375 | C | ASN | 190 | 48.197 | 11.061 | 94.918 | 1.00 | 64.94 | A |
| ATOM | 1376 | O | ASN | 190 | 47.182 | 11.662 | 94.565 | 1.00 | 64.53 | A |
| ATOM | 1377 | N | LYS | 191 | 48.669 | 9.999 | 94.273 | 1.00 | 64.72 | A |
| ATOM | 1378 | CA | LYS | 191 | 48.018 | 9.463 | 93.083 | 1.00 | 63.98 | A |
| ATOM | 1379 | CB | LYS | 191 | 48.810 | 8.266 | 92.541 | 1.00 | 65.18 | A |
| ATOM | 1380 | CG | LYS | 191 | 48.799 | 7.041 | 93.447 | 1.00 | 66.13 | A |
| ATOM | 1381 | CD | LYS | 191 | 49.405 | 5.830 | 92.747 | 1.00 | 67.02 | A |
| ATOM | 1382 | CE | LYS | 191 | 49.274 | 4.572 | 93.593 | 1.00 | 68.29 | A |
| ATOM | 1383 | NZ | LYS | 191 | 49.860 | 3.375 | 92.919 | 1.00 | 69.29 | A |
| ATOM | 1384 | C | LYS | 191 | 46.577 | 9.039 | 93.358 | 1.00 | 62.26 | A |
| ATOM | 1385 | O | LYS | 191 | 46.151 | 8.963 | 94.513 | 1.00 | 63.17 | A |
| ATOM | 1386 | N | ARG | 192 | 45.843 | 8.756 | 92.282 | 1.00 | 58.36 | A |
| ATOM | 1387 | CA | ARG | 192 | 44.440 | 8.350 | 92.348 | 1.00 | 54.26 | A |
| ATOM | 1388 | CB | ARG | 192 | 44.308 | 6.833 | 92.578 | 1.00 | 56.88 | A |
| ATOM | 1389 | CG | ARG | 192 | 44.776 | 6.289 | 93.926 | 1.00 | 59.69 | A |
| ATOM | 1390 | CD | ARG | 192 | 43.939 | 5.062 | 94.306 | 1.00 | 62.18 | A |
| ATOM | 1391 | NE | ARG | 192 | 44.633 | 4.121 | 95.181 | 1.00 | 64.60 | A |
| ATOM | 1392 | CZ | ARG | 192 | 45.640 | 3.344 | 94.792 | 1.00 | 66.61 | A |
| ATOM | 1393 | NH1 | ARG | 192 | 46.074 | 3.400 | 93.539 | 1.00 | 66.97 | A |
| ATOM | 1394 | NH2 | ARG | 192 | 46.209 | 2.505 | 95.650 | 1.00 | 67.30 | A |
| ATOM | 1395 | C | ARG | 192 | 43.619 | 9.106 | 93.391 | 1.00 | 50.08 | A |
| ATOM | 1396 | O | ARG | 192 | 42.742 | 8.538 | 94.049 | 1.00 | 50.87 | A |
| ATOM | 1397 | N | GLY | 193 | 43.909 | 10.395 | 93.531 | 1.00 | 44.14 | A |
| ATOM | 1398 | CA | GLY | 193 | 43.183 | 11.231 | 94.469 | 1.00 | 35.61 | A |
| ATOM | 1399 | C | GLY | 193 | 42.799 | 12.482 | 93.712 | 1.00 | 30.34 | A |
| ATOM | 1400 | O | GLY | 193 | 43.343 | 12.732 | 92.639 | 1.00 | 30.32 | A |
| ATOM | 1401 | N | VAL | 194 | 41.865 | 13.264 | 94.238 | 1.00 | 25.49 | A |
| ATOM | 1402 | CA | VAL | 194 | 41.463 | 14.489 | 93.557 | 1.00 | 21.22 | A |
| ATOM | 1403 | CB | VAL | 194 | 40.078 | 14.359 | 92.884 | 1.00 | 20.31 | A |
| ATOM | 1404 | CG1 | VAL | 194 | 40.100 | 13.289 | 91.809 | 1.00 | 19.29 | A |
| ATOM | 1405 | CG2 | VAL | 194 | 39.032 | 14.059 | 93.935 | 1.00 | 18.96 | A |
| ATOM | 1406 | C | VAL | 194 | 41.375 | 15.668 | 94.505 | 1.00 | 20.08 | A |
| ATOM | 1407 | O | VAL | 194 | 41.417 | 15.515 | 95.722 | 1.00 | 20.27 | A |
| ATOM | 1408 | N | ILE | 195 | 41.238 | 16.853 | 93.930 | 1.00 | 20.12 | A |
| ATOM | 1409 | CA | ILE | 195 | 41.109 | 18.065 | 94.713 | 1.00 | 18.57 | A |
| ATOM | 1410 | CB | ILE | 195 | 42.298 | 19.014 | 94.477 | 1.00 | 20.69 | A |
| ATOM | 1411 | CG2 | ILE | 195 | 42.011 | 20.362 | 95.118 | 1.00 | 21.74 | A |
| ATOM | 1412 | CG1 | ILE | 195 | 43.584 | 18.392 | 95.029 | 1.00 | 21.99 | A |
| ATOM | 1413 | CD1 | ILE | 195 | 44.853 | 19.212 | 94.722 | 1.00 | 23.27 | A |
| ATOM | 1414 | C | ILE | 195 | 39.838 | 18.791 | 94.297 | 1.00 | 17.41 | A |
| ATOM | 1415 | O | ILE | 195 | 39.639 | 19.077 | 93.115 | 1.00 | 15.50 | A |
| ATOM | 1416 | N | ILE | 196 | 38.962 | 19.066 | 95.256 | 1.00 | 17.01 | A |
| ATOM | 1417 | CA | ILE | 196 | 37.751 | 19.805 | 94.939 | 1.00 | 18.54 | A |
| ATOM | 1418 | CB | ILE | 196 | 36.493 | 19.251 | 95.639 | 1.00 | 18.28 | A |
| ATOM | 1419 | CG2 | ILE | 196 | 35.299 | 20.143 | 95.314 | 1.00 | 13.69 | A |
| ATOM | 1420 | CG1 | ILE | 196 | 36.209 | 17.819 | 95.171 | 1.00 | 17.38 | A |
| ATOM | 1421 | CD1 | ILE | 196 | 37.016 | 16.775 | 95.894 | 1.00 | 21.62 | A |
| ATOM | 1422 | C | ILE | 196 | 37.981 | 21.232 | 95.407 | 1.00 | 20.22 | A |
| ATOM | 1423 | O | ILE | 196 | 38.001 | 21.517 | 96.606 | 1.00 | 20.32 | A |
| ATOM | 1424 | N | LYS | 197 | 38.158 | 22.122 | 94.441 | 1.00 | 21.72 | A |
| ATOM | 1425 | CA | LYS | 197 | 38.418 | 23.524 | 94.709 | 1.00 | 23.72 | A |
| ATOM | 1426 | CB | LYS | 197 | 38.807 | 24.209 | 93.397 | 1.00 | 26.40 | A |
| ATOM | 1427 | CG | LYS | 197 | 39.068 | 25.693 | 93.481 | 1.00 | 29.01 | A |
| ATOM | 1428 | CD | LYS | 197 | 39.519 | 26.211 | 92.125 | 1.00 | 32.62 | A |
| ATOM | 1429 | CE | LYS | 197 | 39.538 | 27.728 | 92.088 | 1.00 | 33.50 | A |
| ATOM | 1430 | NZ | LYS | 197 | 38.172 | 28.259 | 92.341 | 1.00 | 36.03 | A |
| ATOM | 1431 | C | LYS | 197 | 37.226 | 24.225 | 95.348 | 1.00 | 24.04 | A |
| ATOM | 1432 | O | LYS | 197 | 36.139 | 24.261 | 94.782 | 1.00 | 24.54 | A |
| ATOM | 1433 | N | GLY | 198 | 37.436 | 24.763 | 96.543 | 1.00 | 24.46 | A |
| ATOM | 1434 | CA | GLY | 198 | 36.377 | 25.478 | 97.227 | 1.00 | 25.68 | A |
| ATOM | 1435 | C | GLY | 198 | 35.413 | 24.681 | 98.088 | 1.00 | 26.82 | A |
| ATOM | 1436 | O | GLY | 198 | 34.482 | 25.256 | 98.652 | 1.00 | 27.32 | A |
| ATOM | 1437 | N | LEU | 199 | 35.612 | 23.373 | 98.202 | 1.00 | 27.36 | A |
| ATOM | 1438 | CA | LEU | 199 | 34.714 | 22.558 | 99.017 | 1.00 | 27.19 | A |
| ATOM | 1439 | CB | LEU | 199 | 35.008 | 21.068 | 98.819 | 1.00 | 26.21 | A |
| ATOM | 1440 | CG | LEU | 199 | 33.908 | 20.008 | 99.023 | 1.00 | 27.04 | A |
| ATOM | 1441 | CD1 | LEU | 199 | 34.563 | 18.778 | 99.630 | 1.00 | 25.53 | A |
| ATOM | 1442 | CD2 | LEU | 199 | 32.779 | 20.497 | 99.924 | 1.00 | 24.18 | A |
| ATOM | 1443 | C | LEU | 199 | 34.920 | 22.925 | 100.484 | 1.00 | 27.51 | A |
| ATOM | 1444 | O | LEU | 199 | 36.024 | 22.822 | 101.005 | 1.00 | 28.57 | A |
| ATOM | 1445 | N | GLU | 200 | 33.856 | 23.346 | 101.150 | 1.00 | 28.60 | A |
| ATOM | 1446 | CA | GLU | 200 | 33.950 | 23.721 | 102.553 | 1.00 | 31.25 | A |
| ATOM | 1447 | CB | GLU | 200 | 32.788 | 24.644 | 102.935 | 1.00 | 34.22 | A |
| ATOM | 1448 | CG | GLU | 200 | 32.933 | 26.067 | 102.419 | 1.00 | 39.68 | A |
| ATOM | 1449 | CD | GLU | 200 | 34.051 | 26.823 | 103.108 | 1.00 | 42.07 | A |
| ATOM | 1450 | OE1 | GLU | 200 | 33.921 | 27.118 | 104.317 | 1.00 | 44.27 | A |
| ATOM | 1451 | OE2 | GLU | 200 | 35.065 | 27.120 | 102.443 | 1.00 | 44.71 | A |
| ATOM | 1452 | C | GLU | 200 | 33.986 | 22.540 | 103.516 | 1.00 | 30.44 | A |
| ATOM | 1453 | O | GLU | 200 | 33.381 | 21.497 | 103.282 | 1.00 | 28.54 | A |
| ATOM | 1454 | N | GLU | 201 | 34.716 | 22.729 | 104.606 | 1.00 | 30.76 | A |
| ATOM | 1455 | CA | GLU | 201 | 34.841 | 21.730 | 105.649 | 1.00 | 29.99 | A |
| ATOM | 1456 | CB | GLU | 201 | 36.281 | 21.247 | 105.742 | 1.00 | 29.82 | A |
| ATOM | 1457 | CG | GLU | 201 | 36.755 | 20.516 | 104.511 | 1.00 | 32.15 | A |
| ATOM | 1458 | CD | GLU | 201 | 38.156 | 19.977 | 104.676 | 1.00 | 35.25 | A |
| ATOM | 1459 | OE1 | GLU | 201 | 38.408 | 19.298 | 105.699 | 1.00 | 34.69 | A |
| ATOM | 1460 | OE2 | GLU | 201 | 39.000 | 20.227 | 103.786 | 1.00 | 36.53 | A |
| ATOM | 1461 | C | GLU | 201 | 34.439 | 22.418 | 106.943 | 1.00 | 29.40 | A |
| ATOM | 1462 | O | GLU | 201 | 35.183 | 23.248 | 107.465 | 1.00 | 30.31 | A |
| ATOM | 1463 | N | ILE | 202 | 33.256 | 22.089 | 107.449 | 1.00 | 27.91 | A |
| ATOM | 1464 | CA | ILE | 202 | 32.765 | 22.694 | 108.679 | 1.00 | 25.94 | A |
| ATOM | 1465 | CB | ILE | 202 | 31.207 | 22.720 | 108.720 | 1.00 | 27.58 | A |
| ATOM | 1466 | CG2 | ILE | 202 | 30.721 | 23.125 | 110.096 | 1.00 | 24.19 | A |
| ATOM | 1467 | CG1 | ILE | 202 | 30.662 | 23.706 | 107.682 | 1.00 | 28.28 | A |
| ATOM | 1468 | CD1 | ILE | 202 | 30.809 | 23.241 | 106.256 | 1.00 | 30.78 | A |
| ATOM | 1469 | C | ILE | 202 | 33.277 | 21.932 | 109.889 | 1.00 | 25.41 | A |
| ATOM | 1470 | O | ILE | 202 | 33.195 | 20.703 | 109.945 | 1.00 | 25.37 | A |
| ATOM | 1471 | N | THR | 203 | 33.811 | 22.667 | 110.856 | 1.00 | 23.88 | A |
| ATOM | 1472 | CA | THR | 203 | 34.321 | 22.070 | 112.083 | 1.00 | 22.88 | A |
| ATOM | 1473 | CB | THR | 203 | 35.397 | 22.981 | 112.742 | 1.00 | 22.77 | A |
| ATOM | 1474 | OG1 | THR | 203 | 36.542 | 23.064 | 111.883 | 1.00 | 23.19 | A |
| ATOM | 1475 | CG2 | THR | 203 | 35.813 | 22.441 | 114.112 | 1.00 | 19.08 | A |
| ATOM | 1476 | C | THR | 203 | 33.143 | 21.919 | 113.038 | 1.00 | 22.21 | A |
| ATOM | 1477 | O | THR | 203 | 32.385 | 22.867 | 113.242 | 1.00 | 22.47 | A |
| ATOM | 1478 | N | VAL | 204 | 32.977 | 20.728 | 113.606 | 1.00 | 21.39 | A |
| ATOM | 1479 | CA | VAL | 204 | 31.891 | 20.474 | 114.549 | 1.00 | 21.47 | A |
| ATOM | 1480 | CB | VAL | 204 | 31.248 | 19.102 | 114.278 | 1.00 | 20.28 | A |
| ATOM | 1481 | CG1 | VAL | 204 | 30.034 | 18.906 | 115.162 | 1.00 | 21.96 | A |
| ATOM | 1482 | CG2 | VAL | 204 | 30.859 | 19.000 | 112.820 | 1.00 | 20.66 | A |
| ATOM | 1483 | C | VAL | 204 | 32.531 | 20.490 | 115.939 | 1.00 | 23.52 | A |
| ATOM | 1484 | O | VAL | 204 | 33.083 | 19.484 | 116.385 | 1.00 | 24.43 | A |
| ATOM | 1485 | N | HIS | 205 | 32.468 | 21.635 | 116.615 | 1.00 | 23.51 | A |
| ATOM | 1486 | CA | HIS | 205 | 33.088 | 21.782 | 117.933 | 1.00 | 24.78 | A |
| ATOM | 1487 | CB | HIS | 205 | 32.979 | 23.238 | 118.407 | 1.00 | 24.16 | A |
| ATOM | 1488 | CG | HIS | 205 | 33.597 | 24.220 | 117.460 | 1.00 | 28.16 | A |
| ATOM | 1489 | CD2 | HIS | 205 | 34.887 | 24.595 | 117.281 | 1.00 | 28.25 | A |
| ATOM | 1490 | ND1 | HIS | 205 | 32.870 | 24.885 | 116.493 | 1.00 | 29.05 | A |
| ATOM | 1491 | CE1 | HIS | 205 | 33.684 | 25.623 | 115.759 | 1.00 | 27.33 | A |
| ATOM | 1492 | NE2 | HIS | 205 | 34.914 | 25.464 | 116.216 | 1.00 | 28.33 | A |
| ATOM | 1493 | C | HIS | 205 | 32.586 | 20.836 | 119.018 | 1.00 | 24.15 | A |
| ATOM | 1494 | O | HIS | 205 | 33.341 | 20.445 | 119.909 | 1.00 | 24.11 | A |
| ATOM | 1495 | N | ASN | 206 | 31.318 | 20.458 | 118.945 | 1.00 | 25.62 | A |
| ATOM | 1496 | CA | ASN | 206 | 30.758 | 19.552 | 119.939 | 1.00 | 26.43 | A |
| ATOM | 1497 | CB | ASN | 206 | 30.598 | 20.275 | 121.281 | 1.00 | 25.52 | A |
| ATOM | 1498 | CG | ASN | 206 | 29.689 | 21.488 | 121.186 | 1.00 | 26.18 | A |
| ATOM | 1499 | OD1 | ASN | 206 | 28.498 | 21.358 | 120.906 | 1.00 | 28.63 | A |
| ATOM | 1500 | ND2 | ASN | 206 | 30.246 | 22.671 | 121.414 | 1.00 | 24.14 | A |
| ATOM | 1501 | C | ASN | 206 | 29.422 | 18.960 | 119.496 | 1.00 | 27.20 | A |
| ATOM | 1502 | O | ASN | 206 | 28.804 | 19.416 | 118.533 | 1.00 | 27.37 | A |
| ATOM | 1503 | N | LYS | 207 | 28.993 | 17.933 | 120.212 | 1.00 | 27.93 | A |
| ATOM | 1504 | CA | LYS | 207 | 27.751 | 17.243 | 119.924 | 1.00 | 30.13 | A |
| ATOM | 1505 | CB | LYS | 207 | 27.449 | 16.252 | 121.060 | 1.00 | 32.58 | A |
| ATOM | 1506 | CG | LYS | 207 | 26.151 | 15.481 | 120.906 | 1.00 | 36.84 | A |
| ATOM | 1507 | CD | LYS | 207 | 25.112 | 15.921 | 121.929 | 1.00 | 40.39 | A |
| ATOM | 1508 | CE | LYS | 207 | 25.525 | 15.543 | 123.349 | 1.00 | 41.61 | A |
| ATOM | 1509 | NZ | LYS | 207 | 24.489 | 15.948 | 124.350 | 1.00 | 43.85 | A |
| ATOM | 1510 | C | LYS | 207 | 26.571 | 18.196 | 119.725 | 1.00 | 29.76 | A |
| ATOM | 1511 | O | LYS | 207 | 25.738 | 17.972 | 118.850 | 1.00 | 30.05 | A |
| ATOM | 1512 | N | ASP | 208 | 26.505 | 19.260 | 120.523 | 1.00 | 28.95 | A |
| ATOM | 1513 | CA | ASP | 208 | 25.402 | 20.214 | 120.429 | 1.00 | 27.71 | A |
| ATOM | 1514 | CB | ASP | 208 | 25.280 | 20.981 | 121.751 | 1.00 | 28.92 | A |
| ATOM | 1515 | CG | ASP | 208 | 24.772 | 20.093 | 122.895 | 1.00 | 33.21 | A |
| ATOM | 1516 | OD1 | ASP | 208 | 24.967 | 20.444 | 124.081 | 1.00 | 32.92 | A |
| ATOM | 1517 | OD2 | ASP | 208 | 24.165 | 19.037 | 122.609 | 1.00 | 34.60 | A |
| ATOM | 1518 | C | ASP | 208 | 25.524 | 21.169 | 119.240 | 1.00 | 26.33 | A |
| ATOM | 1519 | O | ASP | 208 | 24.836 | 22.186 | 119.156 | 1.00 | 26.39 | A |
| ATOM | 1520 | N | GLU | 209 | 26.381 | 20.810 | 118.296 | 1.00 | 24.27 | A |
| ATOM | 1521 | CA | GLU | 209 | 26.580 | 21.630 | 117.116 | 1.00 | 21.87 | A |
| ATOM | 1522 | CB | GLU | 209 | 28.039 | 22.074 | 117.066 | 1.00 | 23.60 | A |
| ATOM | 1523 | CG | GLU | 209 | 28.331 | 23.202 | 116.106 | 1.00 | 25.30 | A |
| ATOM | 1524 | CD | GLU | 209 | 29.678 | 23.849 | 116.384 | 1.00 | 25.66 | A |
| ATOM | 1525 | OE1 | GLU | 209 | 29.872 | 24.362 | 117.507 | 1.00 | 25.63 | A |
| ATOM | 1526 | OE2 | GLU | 209 | 30.538 | 23.845 | 115.481 | 1.00 | 26.97 | A |
| ATOM | 1527 | C | GLU | 209 | 26.217 | 20.819 | 115.874 | 1.00 | 19.67 | A |
| ATOM | 1528 | O | GLU | 209 | 26.125 | 21.350 | 114.769 | 1.00 | 18.53 | A |
| ATOM | 1529 | N | VAL | 210 | 25.988 | 19.528 | 116.075 | 1.00 | 16.60 | A |
| ATOM | 1530 | CA | VAL | 210 | 25.648 | 18.625 | 114.985 | 1.00 | 17.06 | A |
| ATOM | 1531 | CB | VAL | 210 | 25.654 | 17.148 | 115.479 | 1.00 | 17.27 | A |
| ATOM | 1532 | CG1 | VAL | 210 | 25.307 | 16.224 | 114.330 | 1.00 | 18.17 | A |
| ATOM | 1533 | CG2 | VAL | 210 | 27.028 | 16.779 | 116.068 | 1.00 | 17.55 | A |
| ATOM | 1534 | C | VAL | 210 | 24.305 | 18.895 | 114.270 | 1.00 | 16.45 | A |
| ATOM | 1535 | O | VAL | 210 | 24.267 | 19.119 | 113.063 | 1.00 | 17.67 | A |
| ATOM | 1536 | N | TYR | 211 | 23.203 | 18.882 | 115.003 | 1.00 | 14.85 | A |
| ATOM | 1537 | CA | TYR | 211 | 21.911 | 19.072 | 114.366 | 1.00 | 15.99 | A |
| ATOM | 1538 | CB | TYR | 211 | 20.789 | 19.050 | 115.404 | 1.00 | 14.76 | A |
| ATOM | 1539 | CG | TYR | 211 | 19.431 | 18.850 | 114.780 | 1.00 | 14.73 | A |
| ATOM | 1540 | CD1 | TYR | 211 | 19.179 | 17.755 | 113.953 | 1.00 | 12.63 | A |
| ATOM | 1541 | CE1 | TYR | 211 | 17.923 | 17.557 | 113.387 | 1.00 | 14.15 | A |
| ATOM | 1542 | CD2 | TYR | 211 | 18.395 | 19.746 | 115.025 | 1.00 | 15.52 | A |
| ATOM | 1543 | CE2 | TYR | 211 | 17.136 | 19.559 | 114.466 | 1.00 | 16.40 | A |
| ATOM | 1544 | CZ | TYR | 211 | 16.903 | 18.462 | 113.649 | 1.00 | 15.49 | A |
| ATOM | 1545 | OH | TYR | 211 | 15.645 | 18.271 | 113.116 | 1.00 | 12.99 | A |
| ATOM | 1546 | C | TYR | 211 | 21.763 | 20.303 | 113.483 | 1.00 | 15.43 | A |
| ATOM | 1547 | O | TYR | 211 | 21.220 | 20.207 | 112.383 | 1.00 | 17.14 | A |
| ATOM | 1548 | N | GLN | 212 | 22.238 | 21.456 | 113.925 | 1.00 | 15.05 | A |
| ATOM | 1549 | CA | GLN | 212 | 22.080 | 22.624 | 113.081 | 1.00 | 17.00 | A |
| ATOM | 1550 | CB | GLN | 212 | 22.384 | 23.912 | 113.855 | 1.00 | 18.93 | A |
| ATOM | 1551 | CG | GLN | 212 | 23.803 | 24.099 | 114.319 | 1.00 | 25.15 | A |
| ATOM | 1552 | CD | GLN | 212 | 23.892 | 25.178 | 115.379 | 1.00 | 29.02 | A |
| ATOM | 1553 | OE1 | GLN | 212 | 23.354 | 26.276 | 115.209 | 1.00 | 30.43 | A |
| ATOM | 1554 | NE2 | GLN | 212 | 24.562 | 24.870 | 116.486 | 1.00 | 30.19 | A |
| ATOM | 1555 | C | GLN | 212 | 22.903 | 22.543 | 111.799 | 1.00 | 16.71 | A |
| ATOM | 1556 | O | GLN | 212 | 22.459 | 23.030 | 110.749 | 1.00 | 16.05 | A |
| ATOM | 1557 | N | ILE | 213 | 24.077 | 21.913 | 111.865 | 1.00 | 14.80 | A |
| ATOM | 1558 | CA | ILE | 213 | 24.921 | 21.776 | 110.678 | 1.00 | 13.74 | A |
| ATOM | 1559 | CB | ILE | 213 | 26.309 | 21.148 | 111.036 | 1.00 | 14.83 | A |
| ATOM | 1560 | CG2 | ILE | 213 | 27.118 | 20.846 | 109.764 | 1.00 | 11.99 | A |
| ATOM | 1561 | CG1 | ILE | 213 | 27.099 | 22.122 | 111.926 | 1.00 | 13.49 | A |
| ATOM | 1562 | CD1 | ILE | 213 | 28.495 | 21.607 | 112.366 | 1.00 | 12.70 | A |
| ATOM | 1563 | C | ILE | 213 | 24.170 | 20.909 | 109.662 | 1.00 | 14.25 | A |
| ATOM | 1564 | O | ILE | 213 | 24.135 | 21.223 | 108.474 | 1.00 | 14.16 | A |
| ATOM | 1565 | N | LEU | 214 | 23.546 | 19.838 | 110.142 | 1.00 | 12.87 | A |
| ATOM | 1566 | CA | LEU | 214 | 22.778 | 18.968 | 109.273 | 1.00 | 13.78 | A |
| ATOM | 1567 | CB | LEU | 214 | 22.355 | 17.705 | 110.022 | 1.00 | 11.53 | A |
| ATOM | 1568 | CG | LEU | 214 | 23.467 | 16.843 | 110.623 | 1.00 | 10.45 | A |
| ATOM | 1569 | CD1 | LEU | 214 | 22.840 | 15.626 | 111.257 | 1.00 | 10.08 | A |
| ATOM | 1570 | CD2 | LEU | 214 | 24.454 | 16.418 | 109.552 | 1.00 | 9.12 | A |
| ATOM | 1571 | C | LEU | 214 | 21.536 | 19.695 | 108.749 | 1.00 | 16.52 | A |
| ATOM | 1572 | O | LEU | 214 | 21.172 | 19.527 | 107.591 | 1.00 | 19.62 | A |
| ATOM | 1573 | N | GLU | 215 | 20.881 | 20.495 | 109.590 | 1.00 | 16.71 | A |
| ATOM | 1574 | CA | GLU | 215 | 19.690 | 21.239 | 109.152 | 1.00 | 19.78 | A |
| ATOM | 1575 | CB | GLU | 215 | 19.085 | 22.053 | 110.306 | 1.00 | 19.90 | A |
| ATOM | 1576 | CG | GLU | 215 | 18.435 | 21.249 | 111.418 | 1.00 | 21.54 | A |
| ATOM | 1577 | CD | GLU | 215 | 17.901 | 22.154 | 112.513 | 1.00 | 24.54 | A |
| ATOM | 1578 | OE1 | GLU | 215 | 16.661 | 22.267 | 112.659 | 1.00 | 25.81 | A |
| ATOM | 1579 | OE2 | GLU | 215 | 18.728 | 22.768 | 113.219 | 1.00 | 23.71 | A |
| ATOM | 1580 | C | GLU | 215 | 20.049 | 22.211 | 108.025 | 1.00 | 20.52 | A |
| ATOM | 1581 | O | GLU | 215 | 19.311 | 22.361 | 107.048 | 1.00 | 19.08 | A |
| ATOM | 1582 | N | LYS | 216 | 21.189 | 22.878 | 108.189 | 1.00 | 21.26 | A |
| ATOM | 1583 | CA | LYS | 216 | 21.677 | 23.840 | 107.215 | 1.00 | 22.33 | A |
| ATOM | 1584 | CB | LYS | 216 | 23.046 | 24.367 | 107.656 | 1.00 | 24.51 | A |
| ATOM | 1585 | CG | LYS | 216 | 23.510 | 25.619 | 106.938 | 1.00 | 28.98 | A |
| ATOM | 1586 | CD | LYS | 216 | 22.872 | 26.865 | 107.523 | 1.00 | 33.02 | A |
| ATOM | 1587 | CE | LYS | 216 | 23.331 | 27.078 | 108.959 | 1.00 | 35.90 | A |
| ATOM | 1588 | NZ | LYS | 216 | 24.819 | 27.142 | 109.072 | 1.00 | 37.29 | A |
| ATOM | 1589 | C | LYS | 216 | 21.782 | 23.150 | 105.850 | 1.00 | 22.36 | A |
| ATOM | 1590 | O | LYS | 216 | 21.371 | 23.708 | 104.832 | 1.00 | 23.95 | A |
| ATOM | 1591 | N | GLY | 217 | 22.318 | 21.931 | 105.838 | 1.00 | 20.62 | A |
| ATOM | 1592 | CA | GLY | 217 | 22.458 | 21.193 | 104.595 | 1.00 | 19.15 | A |
| ATOM | 1593 | C | GLY | 217 | 21.119 | 20.836 | 103.976 | 1.00 | 19.07 | A |
| ATOM | 1594 | O | GLY | 217 | 20.938 | 20.932 | 102.760 | 1.00 | 18.70 | A |
| ATOM | 1595 | N | ALA | 218 | 20.168 | 20.431 | 104.812 | 1.00 | 17.10 | A |
| ATOM | 1596 | CA | ALA | 218 | 18.845 | 20.070 | 104.330 | 1.00 | 15.84 | A |
| ATOM | 1597 | CB | ALA | 218 | 17.996 | 19.525 | 105.471 | 1.00 | 14.05 | A |
| ATOM | 1598 | C | ALA | 218 | 18.157 | 21.275 | 103.696 | 1.00 | 15.48 | A |
| ATOM | 1599 | O | ALA | 218 | 17.533 | 21.155 | 102.638 | 1.00 | 15.90 | A |
| ATOM | 1600 | N | ALA | 219 | 18.273 | 22.436 | 104.331 | 1.00 | 14.41 | A |
| ATOM | 1601 | CA | ALA | 219 | 17.638 | 23.642 | 103.800 | 1.00 | 14.13 | A |
| ATOM | 1602 | CB | ALA | 219 | 17.776 | 24.799 | 104.787 | 1.00 | 12.71 | A |
| ATOM | 1603 | C | ALA | 219 | 18.208 | 24.051 | 102.452 | 1.00 | 13.46 | A |
| ATOM | 1604 | O | ALA | 219 | 17.469 | 24.441 | 101.561 | 1.00 | 13.70 | A |
| ATOM | 1605 | N | LYS | 220 | 19.525 | 23.978 | 102.304 | 1.00 | 13.95 | A |
| ATOM | 1606 | CA | LYS | 220 | 20.146 | 24.357 | 101.045 | 1.00 | 14.23 | A |
| ATOM | 1607 | CB | LYS | 220 | 21.666 | 24.380 | 101.192 | 1.00 | 12.72 | A |
| ATOM | 1608 | CG | LYS | 220 | 22.360 | 25.077 | 100.038 | 1.00 | 17.07 | A |
| ATOM | 1609 | CD | LYS | 220 | 23.833 | 25.326 | 100.309 | 1.00 | 15.93 | A |
| ATOM | 1610 | CE | LYS | 220 | 24.512 | 25.923 | 99.080 | 1.00 | 17.58 | A |
| ATOM | 1611 | NZ | LYS | 220 | 25.991 | 26.097 | 99.261 | 1.00 | 15.01 | A |
| ATOM | 1612 | C | LYS | 220 | 19.718 | 23.360 | 99.969 | 1.00 | 14.89 | A |
| ATOM | 1613 | O | LYS | 220 | 19.497 | 23.722 | 98.809 | 1.00 | 15.14 | A |
| ATOM | 1614 | N | ARG | 221 | 19.572 | 22.105 | 100.380 | 1.00 | 14.35 | A |
| ATOM | 1615 | CA | ARG | 221 | 19.166 | 21.024 | 99.492 | 1.00 | 15.09 | A |
| ATOM | 1616 | CB | ARG | 221 | 19.185 | 19.714 | 100.274 | 1.00 | 14.48 | A |
| ATOM | 1617 | CG | ARG | 221 | 19.467 | 18.488 | 99.455 | 1.00 | 18.77 | A |
| ATOM | 1618 | CD | ARG | 221 | 19.485 | 17.273 | 100.365 | 1.00 | 20.34 | A |
| ATOM | 1619 | NE | ARG | 221 | 20.806 | 16.655 | 100.446 | 1.00 | 21.59 | A |
| ATOM | 1620 | CZ | ARG | 221 | 21.148 | 15.748 | 101.357 | 1.00 | 21.60 | A |
| ATOM | 1621 | NH1 | ARG | 221 | 20.264 | 15.361 | 102.272 | 1.00 | 19.86 | A |
| ATOM | 1622 | NH2 | ARG | 221 | 22.367 | 15.218 | 101.344 | 1.00 | 19.97 | A |
| ATOM | 1623 | C | ARG | 221 | 17.761 | 21.290 | 98.932 | 1.00 | 15.56 | A |
| ATOM | 1624 | O | ARG | 221 | 17.419 | 20.858 | 97.827 | 1.00 | 15.28 | A |
| ATOM | 1625 | N | THR | 222 | 16.945 | 22.004 | 99.698 | 1.00 | 14.05 | A |
| ATOM | 1626 | CA | THR | 222 | 15.608 | 22.325 | 99.253 | 1.00 | 13.31 | A |
| ATOM | 1627 | CB | THR | 222 | 14.781 | 22.963 | 100.384 | 1.00 | 16.22 | A |
| ATOM | 1628 | OG1 | THR | 222 | 14.707 | 22.058 | 101.495 | 1.00 | 16.19 | A |
| ATOM | 1629 | CG2 | THR | 222 | 13.367 | 23.252 | 99.904 | 1.00 | 17.44 | A |
| ATOM | 1630 | C | THR | 222 | 15.679 | 23.284 | 98.061 | 1.00 | 13.31 | A |
| ATOM | 1631 | O | THR | 222 | 14.850 | 23.205 | 97.156 | 1.00 | 12.26 | A |
| ATOM | 1632 | N | THR | 223 | 16.667 | 24.175 | 98.044 | 1.00 | 11.79 | A |
| ATOM | 1633 | CA | THR | 223 | 16.787 | 25.112 | 96.936 | 1.00 | 13.70 | A |
| ATOM | 1634 | CB | THR | 223 | 17.675 | 26.345 | 97.287 | 1.00 | 14.50 | A |
| ATOM | 1635 | OG1 | THR | 223 | 19.058 | 25.979 | 97.247 | 1.00 | 18.73 | A |
| ATOM | 1636 | CG2 | THR | 223 | 17.343 | 26.870 | 98.669 | 1.00 | 10.63 | A |
| ATOM | 1637 | C | THR | 223 | 17.387 | 24.398 | 95.729 | 1.00 | 15.22 | A |
| ATOM | 1638 | O | THR | 223 | 17.148 | 24.778 | 94.580 | 1.00 | 17.54 | A |
| ATOM | 1639 | N | ALA | 224 | 18.176 | 23.361 | 95.986 | 1.00 | 14.46 | A |
| ATOM | 1640 | CA | ALA | 224 | 18.773 | 22.607 | 94.896 | 1.00 | 13.62 | A |
| ATOM | 1641 | CB | ALA | 224 | 19.793 | 21.615 | 95.432 | 1.00 | 14.83 | A |
| ATOM | 1642 | C | ALA | 224 | 17.665 | 21.867 | 94.171 | 1.00 | 13.10 | A |
| ATOM | 1643 | O | ALA | 224 | 17.672 | 21.775 | 92.958 | 1.00 | 13.24 | A |
| ATOM | 1644 | N | ALA | 225 | 16.710 | 21.346 | 94.932 | 1.00 | 13.91 | A |
| ATOM | 1645 | CA | ALA | 225 | 15.598 | 20.596 | 94.369 | 1.00 | 15.07 | A |
| ATOM | 1646 | CB | ALA | 225 | 14.817 | 19.903 | 95.498 | 1.00 | 15.97 | A |
| ATOM | 1647 | C | ALA | 225 | 14.640 | 21.422 | 93.498 | 1.00 | 14.78 | A |
| ATOM | 1648 | O | ALA | 225 | 14.070 | 20.908 | 92.532 | 1.00 | 13.24 | A |
| ATOM | 1649 | N | THR | 226 | 14.449 | 22.694 | 93.822 | 1.00 | 15.56 | A |
| ATOM | 1650 | CA | THR | 226 | 13.555 | 23.490 | 92.995 | 1.00 | 16.82 | A |
| ATOM | 1651 | CB | THR | 226 | 12.992 | 24.729 | 93.747 | 1.00 | 17.66 | A |
| ATOM | 1652 | OG1 | THR | 226 | 13.314 | 25.921 | 93.015 | 1.00 | 21.16 | A |
| ATOM | 1653 | CG2 | THR | 226 | 13.557 | 24.822 | 95.142 | 1.00 | 16.64 | A |
| ATOM | 1654 | C | THR | 226 | 14.300 | 23.943 | 91.745 | 1.00 | 15.61 | A |
| ATOM | 1655 | O | THR | 226 | 13.685 | 24.257 | 90.726 | 1.00 | 13.81 | A |
| ATOM | 1656 | N | LEU | 227 | 15.629 | 23.947 | 91.828 | 1.00 | 14.58 | A |
| ATOM | 1657 | CA | LEU | 227 | 16.473 | 24.361 | 90.716 | 1.00 | 14.64 | A |
| ATOM | 1658 | CB | LEU | 227 | 17.751 | 24.993 | 91.267 | 1.00 | 17.19 | A |
| ATOM | 1659 | CG | LEU | 227 | 18.827 | 25.459 | 90.285 | 1.00 | 22.76 | A |
| ATOM | 1660 | CD1 | LEU | 227 | 18.209 | 26.283 | 89.160 | 1.00 | 21.40 | A |
| ATOM | 1661 | CD2 | LEU | 227 | 19.873 | 26.272 | 91.055 | 1.00 | 24.08 | A |
| ATOM | 1662 | C | LEU | 227 | 16.808 | 23.223 | 89.742 | 1.00 | 15.20 | A |
| ATOM | 1663 | O | LEU | 227 | 16.939 | 23.453 | 88.540 | 1.00 | 16.19 | A |
| ATOM | 1664 | N | MET | 228 | 16.924 | 22.000 | 90.256 | 1.00 | 13.63 | A |
| ATOM | 1665 | CA | MET | 228 | 17.244 | 20.842 | 89.424 | 1.00 | 14.22 | A |
| ATOM | 1666 | CB | MET | 228 | 18.607 | 20.275 | 89.852 | 1.00 | 17.08 | A |
| ATOM | 1667 | CG | MET | 228 | 19.771 | 21.243 | 89.583 | 1.00 | 18.22 | A |
| ATOM | 1668 | SD | MET | 228 | 21.340 | 20.816 | 90.414 | 1.00 | 19.64 | A |
| ATOM | 1669 | CE | MET | 228 | 21.189 | 21.761 | 91.964 | 1.00 | 16.95 | A |
| ATOM | 1670 | C | MET | 228 | 16.148 | 19.768 | 89.504 | 1.00 | 13.11 | A |
| ATOM | 1671 | O | MET | 228 | 15.683 | 19.423 | 90.588 | 1.00 | 10.34 | A |
| ATOM | 1672 | N | ASN | 229 | 15.748 | 19.243 | 88.348 | 1.00 | 12.86 | A |
| ATOM | 1673 | CA | ASN | 229 | 14.676 | 18.246 | 88.259 | 1.00 | 13.74 | A |
| ATOM | 1674 | CB | ASN | 229 | 14.319 | 17.975 | 86.794 | 1.00 | 13.77 | A |
| ATOM | 1675 | CG | ASN | 229 | 13.993 | 19.241 | 86.023 | 1.00 | 15.98 | A |
| ATOM | 1676 | OD1 | ASN | 229 | 13.899 | 19.221 | 84.790 | 1.00 | 16.80 | A |
| ATOM | 1677 | ND2 | ASN | 229 | 13.814 | 20.352 | 86.740 | 1.00 | 15.44 | A |
| ATOM | 1678 | C | ASN | 229 | 14.976 | 16.915 | 88.930 | 1.00 | 14.79 | A |
| ATOM | 1679 | O | ASN | 229 | 16.036 | 16.322 | 88.713 | 1.00 | 15.96 | A |
| ATOM | 1680 | N | ALA | 230 | 14.022 | 16.444 | 89.728 | 1.00 | 12.65 | A |
| ATOM | 1681 | CA | ALA | 230 | 14.155 | 15.182 | 90.443 | 1.00 | 13.20 | A |
| ATOM | 1682 | CB | ALA | 230 | 13.971 | 14.010 | 89.476 | 1.00 | 11.65 | A |
| ATOM | 1683 | C | ALA | 230 | 15.514 | 15.099 | 91.114 | 1.00 | 12.14 | A |
| ATOM | 1684 | O | ALA | 230 | 16.187 | 14.071 | 91.056 | 1.00 | 11.89 | A |
| ATOM | 1685 | N | TYR | 233 | 15.906 | 16.190 | 91.753 | 1.00 | 11.37 | A |
| ATOM | 1686 | CA | TYR | 231 | 17.190 | 16.270 | 92.435 | 1.00 | 12.67 | A |
| ATOM | 1687 | CB | TYR | 231 | 17.325 | 17.625 | 93.128 | 1.00 | 13.10 | A |
| ATOM | 1688 | CG | TYR | 231 | 18.685 | 17.843 | 93.720 | 1.00 | 13.58 | A |
| ATOM | 1689 | CD1 | TYR | 231 | 18.951 | 17.526 | 95.050 | 1.00 | 15.59 | A |
| ATOM | 1690 | CE1 | TYR | 231 | 20.235 | 17.687 | 95.583 | 1.00 | 15.33 | A |
| ATOM | 1691 | CD2 | TYR | 231 | 19.728 | 18.325 | 92.934 | 1.00 | 14.58 | A |
| ATOM | 1692 | CE2 | TYR | 231 | 21.008 | 18.489 | 93.454 | 1.00 | 15.62 | A |
| ATOM | 1693 | CZ | TYR | 231 | 21.251 | 18.169 | 94.777 | 1.00 | 14.53 | A |
| ATOM | 1694 | OH | TYR | 231 | 22.508 | 18.355 | 95.291 | 1.00 | 16.72 | A |
| ATOM | 1695 | C | TYR | 231 | 17.431 | 15.162 | 93.458 | 1.00 | 12.52 | A |
| ATOM | 1696 | O | TYR | 231 | 18.470 | 14.500 | 93.436 | 1.00 | 12.31 | A |
| ATOM | 1697 | N | SER | 232 | 16.457 | 14.968 | 94.341 | 1.00 | 12.51 | A |
| ATOM | 1698 | CA | SER | 232 | 16.543 | 13.978 | 95.406 | 1.00 | 11.76 | A |
| ATOM | 1699 | CB | SER | 232 | 15.325 | 14.091 | 96.331 | 1.00 | 10.64 | A |
| ATOM | 1700 | OG | SER | 232 | 14.143 | 13.654 | 95.692 | 1.00 | 10.59 | A |
| ATOM | 1701 | C | SER | 232 | 16.691 | 12.534 | 94.936 | 1.00 | 12.25 | A |
| ATOM | 1702 | O | SER | 232 | 17.123 | 11.673 | 95.702 | 1.00 | 12.40 | A |
| ATOM | 1703 | N | SER | 233 | 16.332 | 12.244 | 93.695 | 1.00 | 11.36 | A |
| ATOM | 1704 | CA | SER | 233 | 16.485 | 10.876 | 93.241 | 1.00 | 12.78 | A |
| ATOM | 1705 | CB | SER | 233 | 15.146 | 10.341 | 92.712 | 1.00 | 13.58 | A |
| ATOM | 1706 | OG | SER | 233 | 14.735 | 11.011 | 91.547 | 1.00 | 17.87 | A |
| ATOM | 1707 | C | SER | 233 | 17.598 | 10.719 | 92.199 | 1.00 | 12.96 | A |
| ATOM | 1708 | O | SER | 233 | 18.129 | 9.628 | 92.018 | 1.00 | 12.33 | A |
| ATOM | 1709 | N | ARG | 234 | 17.984 | 11.817 | 91.552 | 1.00 | 13.08 | A |
| ATOM | 1710 | CA | ARG | 234 | 19.022 | 11.770 | 90.519 | 1.00 | 12.98 | A |
| ATOM | 1711 | CB | ARG | 234 | 18.639 | 12.658 | 89.333 | 1.00 | 13.88 | A |
| ATOM | 1712 | CG | ARG | 234 | 17.411 | 12.209 | 88.575 | 1.00 | 15.89 | A |
| ATOM | 1713 | CD | ARG | 234 | 17.135 | 13.146 | 87.408 | 1.00 | 16.18 | A |
| ATOM | 1714 | NE | ARG | 234 | 15.961 | 12.713 | 86.672 | 1.00 | 20.62 | A |
| ATOM | 1715 | CZ | ARG | 234 | 15.330 | 13.442 | 85.761 | 1.00 | 21.81 | A |
| ATOM | 1716 | NH1 | ARG | 234 | 15.764 | 14.662 | 85.459 | 1.00 | 21.30 | A |
| ATOM | 1717 | NH2 | ARG | 234 | 14.249 | 12.951 | 85.168 | 1.00 | 21.53 | A |
| ATOM | 1718 | C | ARG | 234 | 20.409 | 12.182 | 90.972 | 1.00 | 11.75 | A |
| ATOM | 1719 | O | ARG | 234 | 21.374 | 12.011 | 90.230 | 1.00 | 11.05 | A |
| ATOM | 1720 | N | SER | 235 | 20.510 | 12.744 | 92.170 | 1.00 | 9.69 | A |
| ATOM | 1721 | CA | SER | 235 | 21.802 | 13.185 | 92.679 | 1.00 | 9.62 | A |
| ATOM | 1722 | CB | SER | 235 | 21.656 | 14.525 | 93.409 | 1.00 | 9.37 | A |
| ATOM | 1723 | OG | SER | 235 | 20.858 | 14.410 | 94.575 | 1.00 | 9.00 | A |
| ATOM | 1724 | C | SER | 235 | 22.445 | 12.171 | 93.617 | 1.00 | 9.66 | A |
| ATOM | 1725 | O | SER | 235 | 21.768 | 11.317 | 94.190 | 1.00 | 12.40 | A |
| ATOM | 1726 | N | HIS | 236 | 23.762 | 12.287 | 93.758 | 1.00 | 8.64 | A |
| ATOM | 1727 | CA | HIS | 236 | 24.573 | 11.436 | 94.627 | 1.00 | 5.39 | A |
| ATOM | 1728 | CB | HIS | 236 | 25.795 | 10.898 | 93.878 | 1.00 | 6.60 | A |
| ATOM | 1729 | CG | HIS | 236 | 25.474 | 10.085 | 92.666 | 1.00 | 6.36 | A |
| ATOM | 1730 | CD2 | HIS | 236 | 25.516 | 10.398 | 91.350 | 1.00 | 6.40 | A |
| ATOM | 1731 | ND1 | HIS | 236 | 25.109 | 8.758 | 92.732 | 1.00 | 6.26 | A |
| ATOM | 1732 | CE1 | HIS | 236 | 24.945 | 8.287 | 91.509 | 1.00 | 4.95 | A |
| ATOM | 1733 | NE2 | HIS | 236 | 25.186 | 9.261 | 90.652 | 1.00 | 5.93 | A |
| ATOM | 1734 | C | HIS | 236 | 25.092 | 12.348 | 95.732 | 1.00 | 6.58 | A |
| ATOM | 1735 | O | HIS | 236 | 25.676 | 13.396 | 95.446 | 1.00 | 5.89 | A |
| ATOM | 1736 | N | SER | 237 | 24.902 | 11.972 | 96.990 | 1.00 | 7.32 | A |
| ATOM | 1737 | CA | SER | 237 | 25.409 | 12.816 | 98.063 | 1.00 | 7.91 | A |
| ATOM | 1738 | CB | SER | 237 | 24.287 | 13.204 | 99.022 | 1.00 | 8.40 | A |
| ATOM | 1739 | OG | SER | 237 | 23.895 | 12.093 | 99.805 | 1.00 | 12.48 | A |
| ATOM | 1740 | C | SER | 237 | 26.505 | 12.089 | 98.830 | 1.00 | 7.51 | A |
| ATOM | 1741 | O | SER | 237 | 26.365 | 10.916 | 99.179 | 1.00 | 10.56 | A |
| ATOM | 1742 | N | VAL | 238 | 27.593 | 12.794 | 99.092 | 1.00 | 7.01 | A |
| ATOM | 1743 | CA | VAL | 238 | 28.714 | 12.236 | 99.822 | 1.00 | 7.37 | A |
| ATOM | 1744 | CB | VAL | 238 | 30.032 | 12.305 | 98.998 | 1.00 | 8.80 | A |
| ATOM | 1745 | CG1 | VAL | 238 | 31.145 | 11.578 | 99.741 | 1.00 | 6.78 | A |
| ATOM | 1746 | CG2 | VAL | 238 | 29.833 | 11.711 | 97.603 | 1.00 | 5.26 | A |
| ATOM | 1747 | C | VAL | 238 | 28.938 | 13.025 | 101.107 | 1.00 | 8.29 | A |
| ATOM | 1748 | O | VAL | 238 | 29.445 | 14.141 | 101.057 | 1.00 | 8.87 | A |
| ATOM | 1749 | N | PHE | 239 | 28.549 | 12.454 | 102.247 | 1.00 | 7.65 | A |
| ATOM | 1750 | CA | PHE | 239 | 28.756 | 13.114 | 103.531 | 1.00 | 7.41 | A |
| ATOM | 1751 | CB | PHE | 239 | 27.557 | 12.895 | 104.454 | 1.00 | 7.34 | A |
| ATOM | 1752 | CG | PHE | 239 | 27.615 | 13.694 | 105.726 | 1.00 | 6.91 | A |
| ATOM | 1753 | CD1 | PHE | 239 | 28.508 | 13.355 | 106.744 | 1.00 | 7.70 | A |
| ATOM | 1754 | CD2 | PHE | 239 | 26.778 | 14.788 | 105.906 | 1.00 | 6.68 | A |
| ATOM | 1755 | CE1 | PHE | 239 | 28.567 | 14.102 | 107.931 | 1.00 | 7.54 | A |
| ATOM | 1756 | CE2 | PHE | 239 | 26.828 | 15.546 | 107.086 | 1.00 | 8.52 | A |
| ATOM | 1757 | CZ | PHE | 239 | 27.724 | 15.201 | 108.101 | 1.00 | 7.57 | A |
| ATOM | 1758 | C | PHE | 239 | 30.016 | 12.525 | 104.169 | 1.00 | 10.17 | A |
| ATOM | 1759 | O | PHE | 239 | 30.063 | 11.334 | 104.486 | 1.00 | 10.87 | A |
| ATOM | 1760 | N | SER | 240 | 31.036 | 13.356 | 104.350 | 1.00 | 9.89 | A |
| ATOM | 1761 | CA | SER | 240 | 32.283 | 12.893 | 104.926 | 1.00 | 11.46 | A |
| ATOM | 1762 | CB | SER | 240 | 33.441 | 13.168 | 103.966 | 1.00 | 10.05 | A |
| ATOM | 1763 | OG | SER | 240 | 33.183 | 12.621 | 102.681 | 1.00 | 14.59 | A |
| ATOM | 1764 | C | SER | 240 | 32.598 | 13.508 | 106.285 | 1.00 | 12.92 | A |
| ATOM | 1765 | O | SER | 240 | 32.405 | 14.705 | 106.509 | 1.00 | 12.61 | A |
| ATOM | 1766 | N | VAL | 241 | 33.078 | 12.665 | 107.193 | 1.00 | 12.52 | A |
| ATOM | 1767 | CA | VAL | 241 | 33.468 | 13.113 | 108.511 | 1.00 | 13.59 | A |
| ATOM | 1768 | CB | VAL | 241 | 32.559 | 12.501 | 109.613 | 1.00 | 14.83 | A |
| ATOM | 1769 | CG1 | VAL | 241 | 32.526 | 10.991 | 109.492 | 1.00 | 17.21 | A |
| ATOM | 1770 | CG2 | VAL | 241 | 33.054 | 12.922 | 110.993 | 1.00 | 13.88 | A |
| ATOM | 1771 | C | VAL | 241 | 34.931 | 12.718 | 108.731 | 1.00 | 13.59 | A |
| ATOM | 1772 | O | VAL | 241 | 35.305 | 11.548 | 108.607 | 1.00 | 10.71 | A |
| ATOM | 1773 | N | THR | 242 | 35.759 | 13.715 | 109.024 | 1.00 | 14.44 | A |
| ATOM | 1774 | CA | THR | 242 | 37.175 | 13.489 | 109.264 | 1.00 | 15.80 | A |
| ATOM | 1775 | CB | THR | 242 | 38.051 | 14.421 | 108.409 | 1.00 | 16.64 | A |
| ATOM | 1776 | OG1 | THR | 242 | 37.719 | 14.238 | 107.025 | 1.00 | 19.41 | A |
| ATOM | 1777 | CG2 | THR | 242 | 39.539 | 14.102 | 108.618 | 1.00 | 11.48 | A |
| ATOM | 1778 | C | THR | 242 | 37.479 | 13.726 | 110.734 | 1.00 | 17.79 | A |
| ATOM | 1779 | O | THR | 242 | 37.051 | 14.719 | 111.322 | 1.00 | 19.50 | A |
| ATOM | 1780 | N | ILE | 243 | 38.224 | 12.805 | 111.326 | 1.00 | 18.66 | A |
| ATOM | 1781 | CA | ILE | 243 | 38.563 | 12.904 | 112.730 | 1.00 | 20.82 | A |
| ATOM | 1782 | CB | ILE | 243 | 37.972 | 11.714 | 113.500 | 1.00 | 20.34 | A |
| ATOM | 1783 | CG2 | ILE | 243 | 38.085 | 11.953 | 114.993 | 1.00 | 20.79 | A |
| ATOM | 1784 | CG1 | ILE | 243 | 36.506 | 11.524 | 113.114 | 1.00 | 21.41 | A |
| ATOM | 1785 | CD1 | ILE | 243 | 35.902 | 10.213 | 113.632 | 1.00 | 20.85 | A |
| ATOM | 1786 | C | ILE | 243 | 40.076 | 12.928 | 112.958 | 1.00 | 23.56 | A |
| ATOM | 1787 | O | ILE | 243 | 40.782 | 11.953 | 112.664 | 1.00 | 23.06 | A |
| ATOM | 1788 | N | HIS | 244 | 40.574 | 14.053 | 113.458 | 1.00 | 25.26 | A |
| ATOM | 1789 | CA | HIS | 244 | 41.994 | 14.177 | 113.765 | 1.00 | 27.63 | A |
| ATOM | 1790 | CB | HIS | 244 | 42.507 | 15.589 | 113.485 | 1.00 | 28.72 | A |
| ATOM | 1791 | CG | HIS | 244 | 42.974 | 15.799 | 112.079 | 1.00 | 32.69 | A |
| ATOM | 1792 | CD2 | HIS | 244 | 44.219 | 15.803 | 111.544 | 1.00 | 33.88 | A |
| ATOM | 1793 | ND1 | HIS | 244 | 42.111 | 16.067 | 111.038 | 1.00 | 34.05 | A |
| ATOM | 1794 | CE1 | HIS | 244 | 42.803 | 16.231 | 109.924 | 1.00 | 33.87 | A |
| ATOM | 1795 | NE2 | HIS | 244 | 44.085 | 16.075 | 110.203 | 1.00 | 35.45 | A |
| ATOM | 1796 | C | HIS | 244 | 42.108 | 13.878 | 115.254 | 1.00 | 29.05 | A |
| ATOM | 1797 | O | HIS | 244 | 41.541 | 14.599 | 116.084 | 1.00 | 28.16 | A |
| ATOM | 1798 | N | MET | 245 | 42.827 | 12.813 | 115.592 | 1.00 | 29.99 | A |
| ATOM | 1799 | CA | MET | 245 | 42.968 | 12.425 | 116.988 | 1.00 | 32.41 | A |
| ATOM | 1800 | CB | MET | 245 | 42.330 | 11.053 | 117.210 | 1.00 | 30.98 | A |
| ATOM | 1801 | CG | MET | 245 | 40.880 | 10.959 | 116.795 | 1.00 | 29.47 | A |
| ATOM | 1802 | SD | MET | 245 | 40.390 | 9.243 | 116.608 | 1.00 | 28.28 | A |
| ATOM | 1803 | CE | MET | 245 | 41.018 | 8.925 | 114.953 | 1.00 | 26.37 | A |
| ATOM | 1804 | C | MET | 245 | 44.395 | 12.388 | 117.520 | 1.00 | 34.03 | A |
| ATOM | 1805 | O | MET | 245 | 45.332 | 11.978 | 116.831 | 1.00 | 33.45 | A |
| ATOM | 1806 | N | LYS | 246 | 44.536 | 12.821 | 118.765 | 1.00 | 36.79 | A |
| ATOM | 1807 | CA | LYS | 246 | 45.813 | 12.813 | 119.456 | 1.00 | 41.41 | A |
| ATOM | 1808 | CB | LYS | 246 | 46.345 | 14.234 | 119.645 | 1.00 | 44.53 | A |
| ATOM | 1809 | CG | LYS | 246 | 47.765 | 14.284 | 120.187 | 1.00 | 48.98 | A |
| ATOM | 1810 | CD | LYS | 246 | 48.360 | 15.678 | 120.048 | 1.00 | 52.77 | A |
| ATOM | 1811 | CE | LYS | 246 | 49.830 | 15.693 | 120.448 | 1.00 | 55.09 | A |
| ATOM | 1812 | NZ | LYS | 246 | 50.445 | 17.035 | 120.232 | 1.00 | 56.33 | A |
| ATOM | 1813 | C | LYS | 246 | 45.496 | 12.179 | 120.799 | 1.00 | 42.14 | A |
| ATOM | 1814 | O | LYS | 246 | 45.157 | 12.860 | 121.764 | 1.00 | 42.94 | A |
| ATOM | 1815 | N | GLU | 247 | 45.586 | 10.859 | 120.834 | 1.00 | 42.88 | A |
| ATOM | 1816 | CA | GLU | 247 | 45.286 | 10.090 | 122.027 | 1.00 | 45.27 | A |
| ATOM | 1817 | CB | GLU | 247 | 44.896 | 8.669 | 121.623 | 1.00 | 45.22 | A |
| ATOM | 1818 | CG | GLU | 247 | 44.301 | 7.829 | 122.726 | 1.00 | 45.70 | A |
| ATOM | 1819 | CD | GLU | 247 | 44.075 | 6.396 | 122.282 | 1.00 | 47.91 | A |
| ATOM | 1820 | OE1 | GLU | 247 | 43.507 | 6.194 | 121.186 | 1.00 | 48.39 | A |
| ATOM | 1821 | OE2 | GLU | 247 | 44.462 | 5.471 | 123.032 | 1.00 | 47.23 | A |
| ATOM | 1822 | C | GLU | 247 | 46.463 | 10.040 | 122.995 | 1.00 | 46.56 | A |
| ATOM | 1823 | O | GLU | 247 | 47.625 | 10.055 | 122.592 | 1.00 | 46.38 | A |
| ATOM | 1824 | N | THR | 248 | 46.144 | 9.988 | 124.281 | 1.00 | 47.43 | A |
| ATOM | 1825 | CA | THR | 248 | 47.155 | 9.903 | 125.320 | 1.00 | 49.03 | A |
| ATOM | 1826 | CB | THR | 248 | 47.340 | 11.259 | 126.029 | 1.00 | 49.86 | A |
| ATOM | 1827 | OG1 | THR | 248 | 47.733 | 12.245 | 125.066 | 1.00 | 50.38 | A |
| ATOM | 1828 | CG2 | THR | 248 | 48.416 | 11.162 | 127.104 | 1.00 | 49.64 | A |
| ATOM | 1829 | C | THR | 248 | 46.679 | 8.838 | 126.309 | 1.00 | 49.49 | A |
| ATOM | 1830 | O | THR | 248 | 45.810 | 9.087 | 127.148 | 1.00 | 49.04 | A |
| ATOM | 1831 | N | THR | 249 | 47.244 | 7.641 | 126.177 | 1.00 | 50.47 | A |
| ATOM | 1832 | CA | THR | 249 | 46.892 | 6.510 | 127.025 | 1.00 | 51.50 | A |
| ATOM | 1833 | CB | THR | 249 | 47.684 | 5.252 | 126.621 | 1.00 | 51.30 | A |
| ATOM | 1834 | OG1 | THR | 249 | 49.072 | 5.435 | 126.933 | 1.00 | 50.45 | A |
| ATOM | 1835 | CG2 | THR | 249 | 47.539 | 4.994 | 125.127 | 1.00 | 50.34 | A |
| ATOM | 1836 | C | THR | 249 | 47.157 | 6.813 | 128.493 | 1.00 | 52.76 | A |
| ATOM | 1837 | O | THR | 249 | 47.801 | 7.811 | 128.819 | 1.00 | 52.66 | A |
| ATOM | 1838 | N | ILE | 250 | 46.663 | 5.948 | 129.375 | 1.00 | 53.97 | A |
| ATOM | 1839 | CA | ILE | 250 | 46.842 | 6.136 | 130.812 | 1.00 | 55.19 | A |
| ATOM | 1840 | CB | ILE | 250 | 46.042 | 5.078 | 131.624 | 1.00 | 55.38 | A |
| ATOM | 1841 | CG2 | ILE | 250 | 44.596 | 5.061 | 131.147 | 1.00 | 55.55 | A |
| ATOM | 1842 | CG1 | ILE | 250 | 46.656 | 3.683 | 131.466 | 1.00 | 55.59 | A |
| ATOM | 1843 | CD1 | ILE | 250 | 46.516 | 3.078 | 130.073 | 1.00 | 56.12 | A |
| ATOM | 1844 | C | ILE | 250 | 48.313 | 6.097 | 131.239 | 1.00 | 55.82 | A |
| ATOM | 1845 | O | ILE | 250 | 48.634 | 6.316 | 132.408 | 1.00 | 55.54 | A |
| ATOM | 1846 | N | ASP | 251 | 49.198 | 5.833 | 130.281 | 1.00 | 56.61 | A |
| ATOM | 1847 | CA | ASP | 251 | 50.633 | 5.776 | 130.543 | 1.00 | 57.44 | A |
| ATOM | 1848 | CB | ASP | 251 | 51.285 | 4.696 | 129.679 | 1.00 | 57.92 | A |
| ATOM | 1849 | CG | ASP | 251 | 50.757 | 3.306 | 129.979 | 1.00 | 58.92 | A |
| ATOM | 1850 | OD1 | ASP | 251 | 50.894 | 2.427 | 129.098 | 1.00 | 59.53 | A |
| ATOM | 1851 | OD2 | ASP | 251 | 50.217 | 3.088 | 131.089 | 1.00 | 57.67 | A |
| ATOM | 1852 | C | ASP | 251 | 51.271 | 7.124 | 130.222 | 1.00 | 57.89 | A |
| ATOM | 1853 | O | ASP | 251 | 51.858 | 7.770 | 131.090 | 1.00 | 59.32 | A |
| ATOM | 1854 | N | GLY | 252 | 51.141 | 7.537 | 128.967 | 1.00 | 57.36 | A |
| ATOM | 1855 | CA | GLY | 252 | 51.707 | 8.797 | 128.526 | 1.00 | 57.52 | A |
| ATOM | 1856 | C | GLY | 252 | 52.089 | 8.717 | 127.060 | 1.00 | 57.92 | A |
| ATOM | 1857 | O | GLY | 252 | 52.814 | 9.571 | 126.545 | 1.00 | 58.43 | A |
| ATOM | 1858 | N | GLU | 253 | 51.602 | 7.675 | 126.392 | 1.00 | 57.56 | A |
| ATOM | 1859 | CA | GLU | 253 | 51.869 | 7.456 | 124.974 | 1.00 | 57.81 | A |
| ATOM | 1860 | CB | GLU | 253 | 51.552 | 6.006 | 124.598 | 1.00 | 59.90 | A |
| ATOM | 1861 | CG | GLU | 253 | 52.084 | 4.968 | 125.573 | 1.00 | 62.49 | A |
| ATOM | 1862 | CD | GLU | 253 | 51.543 | 3.581 | 125.294 | 1.00 | 63.65 | A |
| ATOM | 1863 | OE1 | GLU | 253 | 51.693 | 3.108 | 124.146 | 1.00 | 65.45 | A |
| ATOM | 1864 | OE2 | GLU | 253 | 50.970 | 2.967 | 126.219 | 1.00 | 63.15 | A |
| ATOM | 1865 | C | GLU | 253 | 50.959 | 8.381 | 124.179 | 1.00 | 56.36 | A |
| ATOM | 1866 | O | GLU | 253 | 49.818 | 8.618 | 124.572 | 1.00 | 56.13 | A |
| ATOM | 1867 | N | GLU | 254 | 51.451 | 8.908 | 123.067 | 1.00 | 54.64 | A |
| ATOM | 1868 | CA | GLU | 254 | 50.626 | 9.790 | 122.256 | 1.00 | 53.82 | A |
| ATOM | 1869 | CB | GLU | 254 | 51.269 | 11.183 | 122.151 | 1.00 | 54.89 | A |
| ATOM | 1870 | CG | GLU | 254 | 52.568 | 11.259 | 121.354 | 1.00 | 56.86 | A |
| ATOM | 1871 | CD | GLU | 254 | 52.363 | 11.790 | 119.939 | 1.00 | 58.42 | A |
| ATOM | 1872 | OE1 | GLU | 254 | 51.856 | 12.924 | 119.800 | 1.00 | 58.67 | A |
| ATOM | 1873 | OE2 | GLU | 254 | 52.713 | 11.078 | 118.968 | 1.00 | 57.93 | A |
| ATOM | 1874 | C | GLU | 254 | 50.397 | 9.186 | 120.876 | 1.00 | 52.35 | A |
| ATOM | 1875 | O | GLU | 254 | 51.340 | 8.945 | 120.124 | 1.00 | 52.94 | A |
| ATOM | 1876 | N | LEU | 255 | 49.135 | 8.916 | 120.560 | 1.00 | 50.68 | A |
| ATOM | 1877 | CA | LEU | 255 | 48.772 | 8.340 | 119.268 | 1.00 | 48.63 | A |
| ATOM | 1878 | CB | LEU | 255 | 47.828 | 7.142 | 119.439 | 1.00 | 49.85 | A |
| ATOM | 1879 | CG | LEU | 255 | 48.236 | 5.895 | 120.231 | 1.00 | 52.23 | A |
| ATOM | 1880 | CD1 | LEU | 255 | 49.595 | 5.409 | 119.752 | 1.00 | 53.67 | A |
| ATOM | 1881 | CD2 | LEU | 255 | 48.278 | 6.201 | 121.720 | 1.00 | 53.72 | A |
| ATOM | 1882 | C | LEU | 255 | 48.069 | 9.381 | 118.413 | 1.00 | 46.05 | A |
| ATOM | 1883 | O | LEU | 255 | 46.978 | 9.832 | 118.755 | 1.00 | 45.38 | A |
| ATOM | 1884 | N | VAL | 256 | 48.695 | 9.772 | 117.310 | 1.00 | 43.74 | A |
| ATOM | 1885 | CA | VAL | 256 | 48.081 | 10.740 | 116.409 | 1.00 | 41.19 | A |
| ATOM | 1886 | CB | VAL | 256 | 49.084 | 11.791 | 115.943 | 1.00 | 40.17 | A |
| ATOM | 1887 | CG1 | VAL | 256 | 48.442 | 12.680 | 114.897 | 1.00 | 38.91 | A |
| ATOM | 1888 | CG2 | VAL | 256 | 49.543 | 12.614 | 117.132 | 1.00 | 40.08 | A |
| ATOM | 1889 | C | VAL | 256 | 47.533 | 9.994 | 115.200 | 1.00 | 39.59 | A |
| ATOM | 1890 | O | VAL | 256 | 48.276 | 9.619 | 114.291 | 1.00 | 39.95 | A |
| ATOM | 1891 | N | LYS | 257 | 46.221 | 9.780 | 115.212 | 1.00 | 36.47 | A |
| ATOM | 1892 | CA | LYS | 257 | 45.534 | 9.056 | 114.150 | 1.00 | 32.43 | A |
| ATOM | 1893 | CB | LYS | 257 | 44.733 | 7.902 | 114.756 | 1.00 | 31.46 | A |
| ATOM | 1894 | CG | LYS | 257 | 45.525 | 7.024 | 115.710 | 1.00 | 31.17 | A |
| ATOM | 1895 | CD | LYS | 257 | 44.613 | 6.174 | 116.573 | 1.00 | 30.49 | A |
| ATOM | 1896 | CE | LYS | 257 | 43.767 | 7.045 | 117.486 | 1.00 | 31.11 | A |
| ATOM | 1897 | NZ | LYS | 257 | 42.941 | 6.216 | 118.411 | 1.00 | 32.10 | A |
| ATOM | 1898 | C | LYS | 257 | 44.585 | 9.965 | 113.384 | 1.00 | 30.18 | A |
| ATOM | 1899 | O | LYS | 257 | 44.067 | 10.944 | 113.928 | 1.00 | 28.57 | A |
| ATOM | 1900 | N | ILE | 258 | 44.361 | 9.624 | 112.120 | 1.00 | 28.11 | A |
| ATOM | 1901 | CA | ILE | 258 | 43.451 | 10.372 | 111.263 | 1.00 | 26.14 | A |
| ATOM | 1902 | CB | ILE | 258 | 44.223 | 11.174 | 110.209 | 1.00 | 26.23 | A |
| ATOM | 1903 | CG2 | ILE | 258 | 43.265 | 11.782 | 109.205 | 1.00 | 26.22 | A |
| ATOM | 1904 | CG1 | ILE | 258 | 45.027 | 12.271 | 110.904 | 1.00 | 27.27 | A |
| ATOM | 1905 | CD1 | ILE | 258 | 45.828 | 13.155 | 109.943 | 1.00 | 29.18 | A |
| ATOM | 1906 | C | ILE | 258 | 42.493 | 9.400 | 110.573 | 1.00 | 24.09 | A |
| ATOM | 1907 | O | ILE | 258 | 42.912 | 8.562 | 109.772 | 1.00 | 24.80 | A |
| ATOM | 1908 | N | GLY | 259 | 41.208 | 9.509 | 110.899 | 1.00 | 20.82 | A |
| ATOM | 1909 | CA | GLY | 259 | 40.221 | 8.629 | 110.300 | 1.00 | 17.04 | A |
| ATOM | 1910 | C | GLY | 259 | 39.214 | 9.376 | 109.447 | 1.00 | 15.18 | A |
| ATOM | 1911 | O | GLY | 259 | 38.843 | 10.502 | 109.765 | 1.00 | 14.10 | A |
| ATOM | 1912 | N | LYS | 260 | 38.782 | 8.764 | 108.349 | 1.00 | 13.62 | A |
| ATOM | 1913 | CA | LYS | 260 | 37.803 | 9.399 | 107.487 | 1.00 | 13.15 | A |
| ATOM | 1914 | CB | LYS | 260 | 38.480 | 9.983 | 106.247 | 1.00 | 13.95 | A |
| ATOM | 1915 | CG | LYS | 260 | 37.557 | 10.866 | 105.414 | 1.00 | 14.12 | A |
| ATOM | 1916 | CD | LYS | 260 | 38.254 | 11.500 | 104.220 | 1.00 | 14.32 | A |
| ATOM | 1917 | CE | LYS | 260 | 37.256 | 12.328 | 103.410 | 1.00 | 16.28 | A |
| ATOM | 1918 | NZ | LYS | 260 | 37.881 | 13.104 | 102.307 | 1.00 | 14.26 | A |
| ATOM | 1919 | C | LYS | 260 | 36.687 | 8.427 | 107.080 | 1.00 | 13.76 | A |
| ATOM | 1920 | O | LYS | 260 | 36.939 | 7.312 | 106.612 | 1.00 | 14.46 | A |
| ATOM | 1921 | N | LEU | 261 | 35.449 | 8.868 | 107.277 | 1.00 | 11.00 | A |
| ATOM | 1922 | CA | LEU | 261 | 34.281 | 8.067 | 106.954 | 1.00 | 9.03 | A |
| ATOM | 1923 | CB | LEU | 261 | 33.461 | 7.830 | 108.217 | 1.00 | 6.67 | A |
| ATOM | 1924 | CG | LEU | 261 | 32.123 | 7.109 | 108.093 | 1.00 | 3.68 | A |
| ATOM | 1925 | CD1 | LEU | 261 | 32.319 | 5.722 | 107.514 | 1.00 | 2.23 | A |
| ATOM | 1926 | CD2 | LEU | 261 | 31.499 | 7.027 | 109.470 | 1.00 | 3.51 | A |
| ATOM | 1927 | C | LEU | 261 | 33.416 | 8.768 | 105.905 | 1.00 | 10.81 | A |
| ATOM | 1928 | O | LEU | 261 | 32.978 | 9.914 | 106.113 | 1.00 | 9.03 | A |
| ATOM | 1929 | N | ASN | 262 | 33.180 | 8.079 | 104.786 | 1.00 | 8.62 | A |
| ATOM | 1930 | CA | ASN | 262 | 32.360 | 8.608 | 103.702 | 1.00 | 9.89 | A |
| ATOM | 1931 | CB | ASN | 262 | 33.042 | 8.371 | 102.348 | 1.00 | 10.45 | A |
| ATOM | 1932 | CG | ASN | 262 | 34.436 | 8.948 | 102.294 | 1.00 | 14.30 | A |
| ATOM | 1933 | OD1 | ASN | 262 | 35.420 | 8.220 | 102.136 | 1.00 | 16.96 | A |
| ATOM | 1934 | ND2 | ASN | 262 | 34.535 | 10.263 | 102.432 | 1.00 | 9.79 | A |
| ATOM | 1935 | C | ASN | 262 | 31.003 | 7.905 | 103.721 | 1.00 | 9.32 | A |
| ATOM | 1936 | O | ASN | 262 | 30.940 | 6.687 | 103.638 | 1.00 | 10.83 | A |
| ATOM | 1937 | N | LEU | 263 | 29.923 | 8.673 | 103.839 | 1.00 | 8.87 | A |
| ATOM | 1938 | CA | LEU | 263 | 28.572 | 8.108 | 103.874 | 1.00 | 8.66 | A |
| ATOM | 1939 | CB | LEU | 263 | 27.832 | 8.607 | 105.108 | 1.00 | 6.12 | A |
| ATOM | 1940 | CG | LEU | 263 | 28.620 | 8.253 | 106.375 | 1.00 | 8.11 | A |
| ATOM | 1941 | CD1 | LEU | 263 | 27.981 | 8.906 | 107.599 | 1.00 | 8.26 | A |
| ATOM | 1942 | CD2 | LEU | 263 | 28.679 | 6.728 | 106.520 | 1.00 | 5.47 | A |
| ATOM | 1943 | C | LEU | 263 | 27.878 | 8.545 | 102.595 | 1.00 | 10.21 | A |
| ATOM | 1944 | O | LEU | 263 | 27.488 | 9.706 | 102.441 | 1.00 | 12.04 | A |
| ATOM | 1945 | N | VAL | 264 | 27.716 | 7.597 | 101.682 | 1.00 | 9.38 | A |
| ATOM | 1946 | CA | VAL | 264 | 27.161 | 7.891 | 100.378 | 1.00 | 9.77 | A |
| ATOM | 1947 | CB | VAL | 264 | 28.089 | 7.329 | 99.291 | 1.00 | 10.33 | A |
| ATOM | 1948 | CG1 | VAL | 264 | 27.734 | 7.907 | 97.928 | 1.00 | 8.01 | A |
| ATOM | 1949 | CG2 | VAL | 264 | 29.522 | 7.637 | 99.672 | 1.00 | 8.80 | A |
| ATOM | 1950 | C | VAL | 264 | 25.765 | 7.403 | 100.104 | 1.00 | 10.32 | A |
| ATOM | 1951 | O | VAL | 264 | 25.465 | 6.212 | 100.226 | 1.00 | 12.03 | A |
| ATOM | 1952 | N | ASP | 265 | 24.925 | 8.355 | 99.714 | 1.00 | 9.00 | A |
| ATOM | 1953 | CA | ASP | 265 | 23.534 | 8.116 | 99.368 | 1.00 | 6.24 | A |
| ATOM | 1954 | CB | ASP | 265 | 22.650 | 9.211 | 99.985 | 1.00 | 5.48 | A |
| ATOM | 1955 | CG | ASP | 265 | 21.171 | 8.994 | 99.713 | 1.00 | 7.76 | A |
| ATOM | 1956 | OD1 | ASP | 265 | 20.851 | 8.232 | 98.782 | 1.00 | 5.27 | A |
| ATOM | 1957 | OD2 | ASP | 265 | 20.328 | 9.589 | 100.421 | 1.00 | 9.82 | A |
| ATOM | 1958 | C | ASP | 265 | 23.497 | 8.203 | 97.838 | 1.00 | 4.32 | A |
| ATOM | 1959 | O | ASP | 265 | 23.410 | 9.289 | 97.270 | 1.00 | 4.24 | A |
| ATOM | 1960 | N | LEU | 266 | 23.575 | 7.060 | 97.172 | 1.00 | 4.44 | A |
| ATOM | 1961 | CA | LEU | 266 | 23.569 | 7.024 | 95.710 | 1.00 | 5.61 | A |
| ATOM | 1962 | CB | LEU | 266 | 23.941 | 5.616 | 95.222 | 1.00 | 1.02 | A |
| ATOM | 1963 | CG | LEU | 266 | 25.345 | 5.124 | 95.622 | 1.00 | 5.57 | A |
| ATOM | 1964 | CD1 | LEU | 266 | 25.561 | 3.649 | 95.242 | 1.00 | 1.02 | A |
| ATOM | 1965 | CD2 | LEU | 266 | 26.379 | 6.020 | 94.942 | 1.00 | 4.62 | A |
| ATOM | 1966 | C | LEU | 266 | 22.252 | 7.451 | 95.065 | 1.00 | 7.56 | A |
| ATOM | 1967 | O | LEU | 266 | 21.190 | 7.438 | 95.694 | 1.00 | 9.23 | A |
| ATOM | 1968 | N | ALA | 267 | 22.336 | 7.845 | 93.801 | 1.00 | 7.43 | A |
| ATOM | 1969 | CA | ALA | 267 | 21.156 | 8.220 | 93.047 | 1.00 | 6.36 | A |
| ATOM | 1970 | CB | ALA | 267 | 21.572 | 8.756 | 91.687 | 1.00 | 5.05 | A |
| ATOM | 1971 | C | ALA | 267 | 20.324 | 6.945 | 92.877 | 1.00 | 6.99 | A |
| ATOM | 1972 | O | ALA | 267 | 20.844 | 5.840 | 93.020 | 1.00 | 5.27 | A |
| ATOM | 1973 | N | GLY | 268 | 19.042 | 7.105 | 92.571 | 1.00 | 9.81 | A |
| ATOM | 1974 | CA | GLY | 268 | 18.170 | 5.961 | 92.378 | 1.00 | 12.51 | A |
| ATOM | 1975 | C | GLY | 268 | 18.633 | 5.079 | 91.233 | 1.00 | 15.67 | A |
| ATOM | 1976 | O | GLY | 268 | 18.859 | 5.555 | 90.113 | 1.00 | 17.12 | A |
| ATOM | 1977 | N | SER | 269 | 18.755 | 3.786 | 91.516 | 1.00 | 15.31 | A |
| ATOM | 1978 | CA | SER | 269 | 19.220 | 2.802 | 90.543 | 1.00 | 18.23 | A |
| ATOM | 1979 | CB | SER | 269 | 19.677 | 1.554 | 91.293 | 1.00 | 17.50 | A |
| ATOM | 1980 | OG | SER | 269 | 18.596 | 1.027 | 92.043 | 1.00 | 12.64 | A |
| ATOM | 1981 | C | SER | 269 | 18.195 | 2.383 | 89.484 | 1.00 | 20.29 | A |
| ATOM | 1982 | O | SER | 269 | 18.497 | 1.549 | 88.627 | 1.00 | 19.97 | A |
| ATOM | 1983 | N | GLU | 270 | 16.994 | 2.950 | 89.537 | 1.00 | 22.91 | A |
| ATOM | 1984 | CA | GLU | 270 | 15.949 | 2.576 | 88.587 | 1.00 | 26.68 | A |
| ATOM | 1985 | CB | GLU | 270 | 14.563 | 2.958 | 89.136 | 1.00 | 24.65 | A |
| ATOM | 1986 | CG | GLU | 270 | 14.251 | 4.460 | 89.210 | 1.00 | 22.35 | A |
| ATOM | 1987 | CD | GLU | 270 | 14.960 | 5.185 | 90.349 | 1.00 | 21.47 | A |
| ATOM | 1988 | OE1 | GLU | 270 | 15.545 | 4.524 | 91.234 | 1.00 | 18.55 | A |
| ATOM | 1989 | OE2 | GLU | 270 | 14.922 | 6.433 | 90.354 | 1.00 | 22.04 | A |
| ATOM | 1990 | C | GLU | 270 | 16.117 | 3.139 | 87.177 | 1.00 | 31.14 | A |
| ATOM | 1991 | O | GLU | 270 | 16.608 | 4.256 | 86.981 | 1.00 | 30.32 | A |
| ATOM | 1992 | N | ASN | 271 | 15.717 | 2.336 | 86.194 | 1.00 | 36.67 | A |
| ATOM | 1993 | CA | ASN | 271 | 15.799 | 2.730 | 84.793 | 1.00 | 41.70 | A |
| ATOM | 1994 | CB | ASN | 271 | 16.856 | 1.900 | 84.059 | 1.00 | 45.31 | A |
| ATOM | 1995 | CG | ASN | 271 | 17.121 | 2.409 | 82.649 | 1.00 | 49.20 | A |
| ATOM | 1996 | OD1 | ASN | 271 | 17.661 | 3.504 | 82.460 | 1.00 | 50.16 | A |
| ATOM | 1997 | ND2 | ASN | 271 | 16.733 | 1.618 | 81.650 | 1.00 | 50.41 | A |
| ATOM | 1998 | C | ASN | 271 | 14.440 | 2.537 | 84.120 | 1.00 | 42.80 | A |
| ATOM | 1999 | O | ASN | 271 | 13.799 | 1.494 | 84.276 | 1.00 | 44.21 | A |
| ATOM | 2000 | N | ASN | 287 | 17.192 | 11.408 | 81.710 | 1.00 | 47.26 | A |
| ATOM | 2001 | CA | ASN | 287 | 18.348 | 11.168 | 80.854 | 1.00 | 46.49 | A |
| ATOM | 2002 | CB | ASN | 287 | 19.078 | 12.487 | 80.582 | 1.00 | 48.42 | A |
| ATOM | 2003 | CG | ASN | 287 | 18.323 | 13.385 | 79.614 | 1.00 | 51.20 | A |
| ATOM | 2004 | OD1 | ASN | 287 | 18.724 | 14.526 | 79.368 | 1.00 | 51.62 | A |
| ATOM | 2005 | ND2 | ASN | 287 | 17.230 | 12.870 | 79.053 | 1.00 | 50.69 | A |
| ATOM | 2006 | C | ASN | 287 | 19.324 | 10.139 | 81.437 | 1.00 | 45.61 | A |
| ATOM | 2007 | O | ASN | 287 | 18.912 | 9.131 | 82.021 | 1.00 | 45.57 | A |
| ATOM | 2008 | N | ILE | 288 | 20.619 | 10.400 | 81.285 | 1.00 | 42.07 | A |
| ATOM | 2009 | CA | ILE | 288 | 21.634 | 9.471 | 81.771 | 1.00 | 37.70 | A |
| ATOM | 2010 | CB | ILE | 288 | 22.657 | 9.156 | 80.646 | 1.00 | 39.37 | A |
| ATOM | 2011 | CG2 | ILE | 288 | 21.964 | 8.416 | 79.511 | 1.00 | 38.36 | A |
| ATOM | 2012 | CG1 | ILE | 288 | 23.269 | 10.450 | 80.095 | 1.00 | 40.59 | A |
| ATOM | 2013 | CD1 | ILE | 288 | 24.498 | 10.959 | 80.863 | 1.00 | 42.56 | A |
| ATOM | 2014 | C | ILE | 288 | 22.385 | 9.924 | 83.019 | 1.00 | 33.61 | A |
| ATOM | 2015 | O | ILE | 288 | 22.668 | 11.113 | 83.194 | 1.00 | 34.30 | A |
| ATOM | 2016 | N | ASN | 289 | 22.682 | 8.970 | 83.897 | 1.00 | 26.00 | A |
| ATOM | 2017 | CA | ASN | 289 | 23.431 | 9.267 | 85.107 | 1.00 | 19.08 | A |
| ATOM | 2018 | CB | ASN | 289 | 22.810 | 8.599 | 86.334 | 1.00 | 17.79 | A |
| ATOM | 2019 | CG | ASN | 289 | 23.253 | 9.253 | 87.645 | 1.00 | 18.18 | A |
| ATOM | 2020 | OD1 | ASN | 289 | 22.461 | 9.928 | 88.299 | 1.00 | 18.30 | A |
| ATOM | 2021 | ND2 | ASN | 289 | 24.516 | 9.065 | 88.023 | 1.00 | 13.15 | A |
| ATOM | 2022 | C | ASN | 289 | 24.808 | 8.679 | 84.861 | 1.00 | 15.55 | A |
| ATOM | 2023 | O | ASN | 289 | 25.033 | 7.493 | 85.072 | 1.00 | 12.50 | A |
| ATOM | 2024 | N | GLN | 290 | 25.727 | 9.515 | 84.398 | 1.00 | 13.86 | A |
| ATOM | 2025 | CA | GLN | 290 | 27.079 | 9.070 | 84.088 | 1.00 | 12.24 | A |
| ATOM | 2026 | CB | GLN | 290 | 27.896 | 10.253 | 83.560 | 1.00 | 11.18 | A |
| ATOM | 2027 | CG | GLN | 290 | 29.284 | 9.913 | 83.068 | 1.00 | 10.23 | A |
| ATOM | 2028 | CD | GLN | 290 | 29.297 | 8.795 | 82.036 | 1.00 | 11.80 | A |
| ATOM | 2029 | OE1 | GLN | 290 | 28.336 | 8.609 | 81.273 | 1.00 | 12.41 | A |
| ATOM | 2030 | NE2 | GLN | 290 | 30.399 | 8.059 | 81.990 | 1.00 | 10.69 | A |
| ATOM | 2031 | C | GLN | 290 | 27.778 | 8.414 | 85.276 | 1.00 | 11.63 | A |
| ATOM | 2032 | O | GLN | 290 | 28.394 | 7.359 | 85.130 | 1.00 | 12.20 | A |
| ATOM | 2033 | N | SER | 291 | 27.662 | 9.023 | 86.452 | 1.00 | 10.76 | A |
| ATOM | 2034 | CA | SER | 291 | 28.304 | 8.485 | 87.650 | 1.00 | 11.04 | A |
| ATOM | 2035 | CB | SER | 291 | 28.163 | 9.450 | 88.830 | 1.00 | 10.12 | A |
| ATOM | 2036 | OG | SER | 291 | 29.068 | 10.536 | 88.711 | 1.00 | 11.06 | A |
| ATOM | 2037 | C | SER | 291 | 27.753 | 7.131 | 88.043 | 1.00 | 11.79 | A |
| ATOM | 2038 | O | SER | 291 | 28.512 | 6.241 | 88.420 | 1.00 | 14.45 | A |
| ATOM | 2039 | N | LEU | 292 | 26.437 | 6.971 | 87.959 | 1.00 | 11.86 | A |
| ATOM | 2040 | CA | LEU | 292 | 25.805 | 5.709 | 88.312 | 1.00 | 10.53 | A |
| ATOM | 2041 | CB | LEU | 292 | 24.278 | 5.875 | 88.329 | 1.00 | 10.11 | A |
| ATOM | 2042 | CG | LEU | 292 | 23.467 | 4.734 | 88.952 | 1.00 | 11.58 | A |
| ATOM | 2043 | CD1 | LEU | 292 | 23.811 | 4.605 | 90.427 | 1.00 | 9.76 | A |
| ATOM | 2044 | CD2 | LEU | 292 | 21.974 | 5.007 | 88.791 | 1.00 | 11.92 | A |
| ATOM | 2045 | C | LEU | 292 | 26.216 | 4.653 | 87.289 | 1.00 | 10.87 | A |
| ATOM | 2046 | O | LEU | 292 | 26.559 | 3.525 | 87.634 | 1.00 | 12.05 | A |
| ATOM | 2047 | N | LEU | 293 | 26.196 | 5.043 | 86.022 | 1.00 | 11.04 | A |
| ATOM | 2048 | CA | LEU | 293 | 26.566 | 4.165 | 84.929 | 1.00 | 11.19 | A |
| ATOM | 2049 | CB | LEU | 293 | 26.382 | 4.922 | 83.608 | 1.00 | 11.77 | A |
| ATOM | 2050 | CG | LEU | 293 | 25.394 | 4.442 | 82.532 | 1.00 | 15.36 | A |
| ATOM | 2051 | CD1 | LEU | 293 | 24.197 | 3.755 | 83.162 | 1.00 | 13.37 | A |
| ATOM | 2052 | CD2 | LEU | 293 | 24.948 | 5.638 | 81.690 | 1.00 | 11.70 | A |
| ATOM | 2053 | C | LEU | 293 | 28.026 | 3.714 | 85.094 | 1.00 | 13.10 | A |
| ATOM | 2054 | O | LEU | 293 | 28.355 | 2.535 | 84.918 | 1.00 | 13.28 | A |
| ATOM | 2055 | N | THR | 294 | 28.896 | 4.660 | 85.437 | 1.00 | 11.21 | A |
| ATOM | 2056 | CA | THR | 294 | 30.313 | 4.372 | 85.613 | 1.00 | 10.86 | A |
| ATOM | 2057 | CB | THR | 294 | 31.119 | 5.690 | 85.778 | 1.00 | 12.02 | A |
| ATOM | 2058 | OG1 | THR | 294 | 30.934 | 6.497 | 84.611 | 1.00 | 11.95 | A |
| ATOM | 2059 | CG2 | THR | 294 | 32.605 | 5.409 | 85.947 | 1.00 | 8.75 | A |
| ATOM | 2060 | C | THR | 294 | 30.571 | 3.459 | 86.809 | 1.00 | 11.13 | A |
| ATOM | 2061 | O | THR | 294 | 31.416 | 2.563 | 86.735 | 1.00 | 10.49 | A |
| ATOM | 2062 | N | LEU | 295 | 29.843 | 3.686 | 87.906 | 1.00 | 11.70 | A |
| ATOM | 2063 | CA | LEU | 295 | 29.983 | 2.870 | 89.117 | 1.00 | 11.27 | A |
| ATOM | 2064 | CB | LEU | 295 | 29.033 | 3.348 | 90.224 | 1.00 | 10.76 | A |
| ATOM | 2065 | CG | LEU | 295 | 28.993 | 2.535 | 91.529 | 1.00 | 10.99 | A |
| ATOM | 2066 | CD1 | LEU | 295 | 30.352 | 2.540 | 92.214 | 1.00 | 12.41 | A |
| ATOM | 2067 | CD2 | LEU | 295 | 27.950 | 3.126 | 92.458 | 1.00 | 10.86 | A |
| ATOM | 2068 | C | LEU | 295 | 29.683 | 1.424 | 88.788 | 1.00 | 10.80 | A |
| ATOM | 2069 | O | LEU | 295 | 30.365 | 0.521 | 89.252 | 1.00 | 12.59 | A |
| ATOM | 2070 | N | GLY | 296 | 28.652 | 1.205 | 87.986 | 1.00 | 11.95 | A |
| ATOM | 2071 | CA | GLY | 296 | 28.311 | −0.153 | 87.607 | 1.00 | 12.43 | A |
| ATOM | 2072 | C | GLY | 296 | 29.444 | −0.772 | 86.810 | 1.00 | 13.06 | A |
| ATOM | 2073 | O | GLY | 296 | 29.796 | −1.938 | 87.007 | 1.00 | 15.18 | A |
| ATOM | 2074 | N | ARG | 297 | 30.021 | 0.014 | 85.906 | 1.00 | 11.06 | A |
| ATOM | 2075 | CA | ARG | 297 | 31.121 | −0.458 | 85.086 | 1.00 | 9.97 | A |
| ATOM | 2076 | CB | ARG | 297 | 31.369 | 0.517 | 83.943 | 1.00 | 9.77 | A |
| ATOM | 2077 | CG | ARG | 297 | 30.264 | 0.487 | 82.909 | 1.00 | 10.57 | A |
| ATOM | 2078 | CD | ARG | 297 | 30.173 | 1.789 | 82.136 | 1.00 | 8.79 | A |
| ATOM | 2079 | NE | ARG | 297 | 29.014 | 1.776 | 81.259 | 1.00 | 10.33 | A |
| ATOM | 2080 | CZ | ARG | 297 | 28.492 | 2.853 | 80.685 | 1.00 | 9.93 | A |
| ATOM | 2081 | NH1 | ARG | 297 | 29.033 | 4.044 | 80.892 | 1.00 | 10.65 | A |
| ATOM | 2082 | NH2 | ARG | 297 | 27.412 | 2.740 | 79.920 | 1.00 | 7.47 | A |
| ATOM | 2083 | C | ARG | 297 | 32.395 | −0.675 | 85.889 | 1.00 | 9.24 | A |
| ATOM | 2084 | O | ARG | 297 | 33.154 | −1.597 | 85.594 | 1.00 | 10.04 | A |
| ATOM | 2085 | N | VAL | 298 | 32.632 | 0.164 | 86.897 | 1.00 | 6.73 | A |
| ATOM | 2086 | CA | VAL | 298 | 33.823 | 0.009 | 87.734 | 1.00 | 7.78 | A |
| ATOM | 2087 | CB | VAL | 298 | 33.988 | 1.196 | 88.719 | 1.00 | 7.07 | A |
| ATOM | 2088 | CG1 | VAL | 298 | 35.026 | 0.865 | 89.773 | 1.00 | 2.16 | A |
| ATOM | 2089 | CG2 | VAL | 298 | 34.408 | 2.449 | 87.957 | 1.00 | 4.22 | A |
| ATOM | 2090 | C | VAL | 298 | 33.775 | −1.315 | 88.517 | 1.00 | 9.86 | A |
| ATOM | 2091 | O | VAL | 298 | 34.761 | −2.057 | 88.556 | 1.00 | 11.69 | A |
| ATOM | 2092 | N | ILE | 299 | 32.625 | −1.616 | 89.120 | 1.00 | 10.47 | A |
| ATOM | 2093 | CA | ILE | 299 | 32.437 | −2.858 | 89.879 | 1.00 | 10.02 | A |
| ATOM | 2094 | CB | ILE | 299 | 31.004 | −2.910 | 90.488 | 1.00 | 10.33 | A |
| ATOM | 2095 | CG2 | ILE | 299 | 30.710 | −4.280 | 91.095 | 1.00 | 9.07 | A |
| ATOM | 2096 | CG1 | ILE | 299 | 30.869 | −1.821 | 91.558 | 1.00 | 10.35 | A |
| ATOM | 2097 | CD1 | ILE | 299 | 29.445 | −1.587 | 92.019 | 1.00 | 13.51 | A |
| ATOM | 2098 | C | ILE | 299 | 32.659 | −4.070 | 88.972 | 1.00 | 11.19 | A |
| ATOM | 2099 | O | ILE | 299 | 33.341 | −5.019 | 89.348 | 1.00 | 9.09 | A |
| ATOM | 2100 | N | THR | 300 | 32.084 | −4.031 | 87.771 | 1.00 | 14.08 | A |
| ATOM | 2101 | CA | THR | 300 | 32.227 | −5.125 | 86.808 | 1.00 | 13.98 | A |
| ATOM | 2102 | CB | THR | 300 | 31.470 | −4.813 | 85.506 | 1.00 | 13.76 | A |
| ATOM | 2103 | OG1 | THR | 300 | 30.062 | −4.803 | 85.770 | 1.00 | 14.55 | A |
| ATOM | 2104 | CG2 | THR | 300 | 31.783 | −5.848 | 84.436 | 1.00 | 10.43 | A |
| ATOM | 2105 | C | THR | 300 | 33.699 | −5.394 | 86.472 | 1.00 | 16.17 | A |
| ATOM | 2106 | O | THR | 300 | 34.151 | −6.536 | 86.533 | 1.00 | 16.23 | A |
| ATOM | 2107 | N | ALA | 301 | 34.442 | −4.345 | 86.120 | 1.00 | 15.12 | A |
| ATOM | 2108 | CA | ALA | 301 | 35.850 | −4.502 | 85.791 | 1.00 | 14.70 | A |
| ATOM | 2109 | CB | ALA | 301 | 36.449 | −3.157 | 85.362 | 1.00 | 13.94 | A |
| ATOM | 2110 | C | ALA | 301 | 36.622 | −5.068 | 86.985 | 1.00 | 14.94 | A |
| ATOM | 2111 | O | ALA | 301 | 37.512 | −5.893 | 86.819 | 1.00 | 15.20 | A |
| ATOM | 2112 | N | LEU | 302 | 36.282 | −4.620 | 88.188 | 1.00 | 16.14 | A |
| ATOM | 2113 | CA | LEU | 302 | 36.951 | −5.101 | 89.392 | 1.00 | 19.53 | A |
| ATOM | 2114 | CB | LEU | 302 | 36.585 | −4.222 | 90.594 | 1.00 | 19.74 | A |
| ATOM | 2115 | CG | LEU | 302 | 37.221 | −2.830 | 90.688 | 1.00 | 17.91 | A |
| ATOM | 2116 | CD1 | LEU | 302 | 36.558 | −2.045 | 91.802 | 1.00 | 17.40 | A |
| ATOM | 2117 | CD2 | LEU | 302 | 38.717 | −2.963 | 90.948 | 1.00 | 15.50 | A |
| ATOM | 2118 | C | LEU | 302 | 36.643 | −6.564 | 89.717 | 1.00 | 21.83 | A |
| ATOM | 2119 | O | LEU | 302 | 37.533 | −7.302 | 90.127 | 1.00 | 23.13 | A |
| ATOM | 2120 | N | VAL | 303 | 35.398 | −6.993 | 89.535 | 1.00 | 24.49 | A |
| ATOM | 2121 | CA | VAL | 303 | 35.059 | −8.379 | 89.838 | 1.00 | 27.38 | A |
| ATOM | 2122 | CB | VAL | 303 | 33.547 | −8.571 | 90.069 | 1.00 | 26.90 | A |
| ATOM | 2123 | CG1 | VAL | 303 | 33.052 | −7.570 | 91.101 | 1.00 | 26.40 | A |
| ATOM | 2124 | CG2 | VAL | 303 | 32.796 | −8.428 | 88.770 | 1.00 | 29.98 | A |
| ATOM | 2125 | C | VAL | 303 | 35.512 | −9.341 | 88.744 | 1.00 | 30.52 | A |
| ATOM | 2126 | O | VAL | 303 | 35.877 | −10.477 | 89.035 | 1.00 | 31.69 | A |
| ATOM | 2127 | N | GLU | 304 | 35.491 | −8.897 | 87.490 | 1.00 | 32.89 | A |
| ATOM | 2128 | CA | GLU | 304 | 35.921 | −9.750 | 86.389 | 1.00 | 35.74 | A |
| ATOM | 2129 | CB | GLU | 304 | 35.203 | −9.374 | 85.094 | 1.00 | 37.37 | A |
| ATOM | 2130 | CG | GLU | 304 | 33.689 | −9.307 | 85.221 | 1.00 | 39.61 | A |
| ATOM | 2131 | CD | GLU | 304 | 32.999 | −9.146 | 83.876 | 1.00 | 42.09 | A |
| ATOM | 2132 | OE1 | GLU | 304 | 33.515 | −8.380 | 83.028 | 1.00 | 42.71 | A |
| ATOM | 2133 | OE2 | GLU | 304 | 31.939 | −9.775 | 83.671 | 1.00 | 41.78 | A |
| ATOM | 2134 | C | GLU | 304 | 37.426 | −9.604 | 86.206 | 1.00 | 37.86 | A |
| ATOM | 2135 | O | GLU | 304 | 37.996 | −10.078 | 85.227 | 1.00 | 37.10 | A |
| ATOM | 2136 | N | ARG | 305 | 38.054 | −8.937 | 87.169 | 1.00 | 40.46 | A |
| ATOM | 2137 | CA | ARG | 305 | 39.496 | −8.716 | 87.177 | 1.00 | 42.89 | A |
| ATOM | 2138 | CB | ARG | 305 | 40.215 | −10.025 | 87.534 | 1.00 | 45.84 | A |
| ATOM | 2139 | CG | ARG | 305 | 40.201 | −10.328 | 89.040 | 1.00 | 50.55 | A |
| ATOM | 2140 | CD | ARG | 305 | 40.942 | −9.222 | 89.795 | 1.00 | 55.95 | A |
| ATOM | 2141 | NE | ARG | 305 | 40.641 | −9.139 | 91.227 | 1.00 | 60.56 | A |
| ATOM | 2142 | CZ | ARG | 305 | 41.079 | −9.988 | 92.154 | 1.00 | 62.46 | A |
| ATOM | 2143 | NH1 | ARG | 305 | 41.848 | −11.016 | 91.816 | 1.00 | 63.45 | A |
| ATOM | 2144 | NH2 | ARG | 305 | 40.765 | −9.793 | 93.431 | 1.00 | 62.35 | A |
| ATOM | 2145 | C | ARG | 305 | 40.094 | −8.101 | 85.913 | 1.00 | 43.03 | A |
| ATOM | 2146 | O | ARG | 305 | 41.257 | −8.337 | 85.585 | 1.00 | 42.44 | A |
| ATOM | 2147 | N | THR | 306 | 39.292 | −7.300 | 85.218 | 1.00 | 43.37 | A |
| ATOM | 2148 | CA | THR | 306 | 39.728 | −6.607 | 84.009 | 1.00 | 43.89 | A |
| ATOM | 2149 | CB | THR | 306 | 38.553 | −5.823 | 83.373 | 1.00 | 44.73 | A |
| ATOM | 2150 | OG1 | THR | 306 | 37.525 | −6.738 | 82.967 | 1.00 | 46.53 | A |
| ATOM | 2151 | CG2 | THR | 306 | 39.021 | −5.031 | 82.173 | 1.00 | 44.99 | A |
| ATOM | 2152 | C | THR | 306 | 40.816 | −5.616 | 84.428 | 1.00 | 43.35 | A |
| ATOM | 2153 | O | THR | 306 | 40.648 | −4.883 | 85.405 | 1.00 | 44.14 | A |
| ATOM | 2154 | N | PRO | 307 | 41.944 | −5.572 | 83.696 | 1.00 | 42.66 | A |
| ATOM | 2155 | CD | PRO | 307 | 42.230 | −6.282 | 82.436 | 1.00 | 43.08 | A |
| ATOM | 2156 | CA | PRO | 307 | 43.039 | −4.651 | 84.035 | 1.00 | 41.12 | A |
| ATOM | 2157 | CB | PRO | 307 | 44.109 | −4.993 | 83.001 | 1.00 | 41.90 | A |
| ATOM | 2158 | CG | PRO | 307 | 43.302 | −5.410 | 81.811 | 1.00 | 42.89 | A |
| ATOM | 2159 | C | PRO | 307 | 42.661 | −3.165 | 84.023 | 1.00 | 39.78 | A |
| ATOM | 2160 | O | PRO | 307 | 43.151 | −2.384 | 84.847 | 1.00 | 38.90 | A |
| ATOM | 2161 | N | HIS | 308 | 41.789 | −2.773 | 83.099 | 1.00 | 36.76 | A |
| ATOM | 2162 | CA | HIS | 308 | 41.373 | −1.381 | 83.018 | 1.00 | 34.24 | A |
| ATOM | 2163 | CB | HIS | 308 | 41.248 | −0.946 | 81.558 | 1.00 | 35.68 | A |
| ATOM | 2164 | CG | HIS | 308 | 40.936 | 0.507 | 81.395 | 1.00 | 38.11 | A |
| ATOM | 2165 | CD2 | HIS | 308 | 39.847 | 1.134 | 80.888 | 1.00 | 39.53 | A |
| ATOM | 2166 | ND1 | HIS | 308 | 41.794 | 1.503 | 81.809 | 1.00 | 38.73 | A |
| ATOM | 2167 | CE1 | HIS | 308 | 41.249 | 2.682 | 81.565 | 1.00 | 39.88 | A |
| ATOM | 2168 | NE2 | HIS | 308 | 40.067 | 2.486 | 81.006 | 1.00 | 40.19 | A |
| ATOM | 2169 | C | HIS | 308 | 40.052 | −1.120 | 83.737 | 1.00 | 31.65 | A |
| ATOM | 2170 | O | HIS | 308 | 39.009 | −1.661 | 83.362 | 1.00 | 32.49 | A |
| ATOM | 2171 | N | VAL | 309 | 40.117 | −0.282 | 84.769 | 1.00 | 26.89 | A |
| ATOM | 2172 | CA | VAL | 309 | 38.959 | 0.101 | 85.580 | 1.00 | 22.85 | A |
| ATOM | 2173 | CB | VAL | 309 | 39.298 | −0.013 | 87.083 | 1.00 | 22.36 | A |
| ATOM | 2174 | CG1 | VAL | 309 | 38.091 | 0.351 | 87.922 | 1.00 | 22.91 | A |
| ATOM | 2175 | CG2 | VAL | 309 | 39.765 | −1.427 | 87.403 | 1.00 | 22.12 | A |
| ATOM | 2176 | C | VAL | 309 | 38.629 | 1.558 | 85.231 | 1.00 | 20.44 | A |
| ATOM | 2177 | O | VAL | 309 | 39.450 | 2.446 | 85.433 | 1.00 | 19.97 | A |
| ATOM | 2178 | N | PRO | 310 | 37.421 | 1.822 | 84.704 | 1.00 | 17.91 | A |
| ATOM | 2179 | CD | PRO | 310 | 36.413 | 0.834 | 84.277 | 1.00 | 14.72 | A |
| ATOM | 2180 | CA | PRO | 310 | 37.019 | 3.186 | 84.322 | 1.00 | 17.34 | A |
| ATOM | 2181 | CB | PRO | 310 | 35.839 | 2.937 | 83.386 | 1.00 | 15.77 | A |
| ATOM | 2182 | CG | PRO | 310 | 35.214 | 1.699 | 83.978 | 1.00 | 15.26 | A |
| ATOM | 2183 | C | PRO | 310 | 36.689 | 4.227 | 85.404 | 1.00 | 16.65 | A |
| ATOM | 2184 | O | PRO | 310 | 35.673 | 4.908 | 85.317 | 1.00 | 15.99 | A |
| ATOM | 2185 | N | TYR | 311 | 37.557 | 4.368 | 86.402 | 1.00 | 18.31 | A |
| ATOM | 2186 | CA | TYR | 311 | 37.346 | 5.335 | 87.485 | 1.00 | 18.33 | A |
| ATOM | 2187 | CB | TYR | 311 | 38.549 | 5.374 | 88.430 | 1.00 | 18.13 | A |
| ATOM | 2188 | CG | TYR | 311 | 38.826 | 4.115 | 89.209 | 1.00 | 20.50 | A |
| ATOM | 2189 | CD1 | TYR | 311 | 37.943 | 3.660 | 90.194 | 1.00 | 19.61 | A |
| ATOM | 2190 | CE1 | TYR | 311 | 38.242 | 2.538 | 90.957 | 1.00 | 19.17 | A |
| ATOM | 2191 | CD2 | TYR | 311 | 40.008 | 3.407 | 89.005 | 1.00 | 19.30 | A |
| ATOM | 2192 | CE2 | TYR | 311 | 40.314 | 2.290 | 89.759 | 1.00 | 18.88 | A |
| ATOM | 2193 | CZ | TYR | 311 | 39.432 | 1.860 | 90.732 | 1.00 | 20.10 | A |
| ATOM | 2194 | OH | TYR | 311 | 39.754 | 0.749 | 91.480 | 1.00 | 23.13 | A |
| ATOM | 2195 | C | TYR | 311 | 37.150 | 6.753 | 86.969 | 1.00 | 19.65 | A |
| ATOM | 2196 | O | TYR | 311 | 36.288 | 7.485 | 87.449 | 1.00 | 20.71 | A |
| ATOM | 2197 | N | ARG | 312 | 37.967 | 7.140 | 85.995 | 1.00 | 19.46 | A |
| ATOM | 2198 | CA | ARG | 312 | 37.919 | 8.484 | 85.447 | 1.00 | 19.67 | A |
| ATOM | 2199 | CB | ARG | 312 | 39.223 | 8.775 | 84.699 | 1.00 | 24.48 | A |
| ATOM | 2200 | CG | ARG | 312 | 40.470 | 8.521 | 85.534 | 1.00 | 31.49 | A |
| ATOM | 2201 | CD | ARG | 312 | 41.737 | 8.793 | 84.742 | 1.00 | 38.21 | A |
| ATOM | 2202 | NE | ARG | 312 | 41.948 | 10.223 | 84.543 | 1.00 | 41.59 | A |
| ATOM | 2203 | CZ | ARG | 312 | 42.419 | 11.040 | 85.479 | 1.00 | 43.45 | A |
| ATOM | 2204 | NH1 | ARG | 312 | 42.733 | 10.564 | 86.678 | 1.00 | 43.96 | A |
| ATOM | 2205 | NH2 | ARG | 312 | 42.570 | 12.332 | 85.217 | 1.00 | 44.26 | A |
| ATOM | 2206 | C | ARG | 312 | 36.736 | 8.826 | 84.547 | 1.00 | 17.18 | A |
| ATOM | 2207 | O | ARG | 312 | 36.610 | 9.976 | 84.121 | 1.00 | 17.17 | A |
| ATOM | 2208 | N | GLU | 313 | 35.856 | 7.869 | 84.262 | 1.00 | 14.11 | A |
| ATOM | 2209 | CA | GLU | 313 | 34.729 | 8.178 | 83.378 | 1.00 | 11.27 | A |
| ATOM | 2210 | CB | GLU | 313 | 34.258 | 6.911 | 82.646 | 1.00 | 10.67 | A |
| ATOM | 2211 | CG | GLU | 313 | 35.399 | 6.213 | 81.891 | 1.00 | 15.89 | A |
| ATOM | 2212 | CD | GLU | 313 | 34.946 | 5.089 | 80.956 | 1.00 | 19.42 | A |
| ATOM | 2213 | OE1 | GLU | 313 | 35.821 | 4.301 | 80.519 | 1.00 | 20.64 | A |
| ATOM | 2214 | OE2 | GLU | 313 | 33.739 | 4.992 | 80.641 | 1.00 | 19.87 | A |
| ATOM | 2215 | C | GLU | 313 | 33.554 | 8.893 | 84.048 | 1.00 | 9.14 | A |
| ATOM | 2216 | O | GLU | 313 | 32.550 | 9.155 | 83.410 | 1.00 | 8.08 | A |
| ATOM | 2217 | N | SER | 314 | 33.692 | 9.226 | 85.327 | 1.00 | 9.25 | A |
| ATOM | 2218 | CA | SER | 314 | 32.647 | 9.951 | 86.051 | 1.00 | 11.62 | A |
| ATOM | 2219 | CB | SER | 314 | 31.508 | 9.011 | 86.467 | 1.00 | 14.09 | A |
| ATOM | 2220 | OG | SER | 314 | 31.812 | 8.354 | 87.688 | 1.00 | 14.04 | A |
| ATOM | 2221 | C | SER | 314 | 33.233 | 10.604 | 87.298 | 1.00 | 11.57 | A |
| ATOM | 2222 | O | SER | 314 | 34.283 | 10.186 | 87.791 | 1.00 | 12.89 | A |
| ATOM | 2223 | N | LYS | 315 | 32.541 | 11.615 | 87.812 | 1.00 | 12.14 | A |
| ATOM | 2224 | CA | LYS | 315 | 32.981 | 12.340 | 89.002 | 1.00 | 14.40 | A |
| ATOM | 2225 | CB | LYS | 315 | 32.082 | 13.556 | 89.246 | 1.00 | 17.33 | A |
| ATOM | 2226 | CG | LYS | 315 | 32.015 | 14.559 | 88.105 | 1.00 | 19.52 | A |
| ATOM | 2227 | CD | LYS | 315 | 33.175 | 15.536 | 88.143 | 1.00 | 22.04 | A |
| ATOM | 2228 | CE | LYS | 315 | 33.021 | 16.584 | 87.054 | 1.00 | 22.29 | A |
| ATOM | 2229 | NZ | LYS | 315 | 32.991 | 15.922 | 85.724 | 1.00 | 25.05 | A |
| ATOM | 2230 | C | LYS | 315 | 32.952 | 11.461 | 90.253 | 1.00 | 14.36 | A |
| ATOM | 2231 | O | LYS | 315 | 33.899 | 11.459 | 91.042 | 1.00 | 15.78 | A |
| ATOM | 2232 | N | LEU | 316 | 31.859 | 10.723 | 90.430 | 1.00 | 12.10 | A |
| ATOM | 2233 | CA | LEU | 316 | 31.693 | 9.864 | 91.591 | 1.00 | 12.11 | A |
| ATOM | 2234 | CB | LEU | 316 | 30.346 | 9.132 | 91.521 | 1.00 | 11.47 | A |
| ATOM | 2235 | CG | LEU | 316 | 30.052 | 8.165 | 92.673 | 1.00 | 11.12 | A |
| ATOM | 2236 | CD1 | LEU | 316 | 29.755 | 8.941 | 93.947 | 1.00 | 10.52 | A |
| ATOM | 2237 | CD2 | LEU | 316 | 28.867 | 7.294 | 92.313 | 1.00 | 9.92 | A |
| ATOM | 2238 | C | LEU | 316 | 32.816 | 8.846 | 91.790 | 1.00 | 12.47 | A |
| ATOM | 2239 | O | LEU | 316 | 33.346 | 8.720 | 92.892 | 1.00 | 13.63 | A |
| ATOM | 2240 | N | THR | 317 | 33.192 | 8.124 | 90.738 | 1.00 | 13.16 | A |
| ATOM | 2241 | CA | THR | 317 | 34.245 | 7.118 | 90.875 | 1.00 | 12.10 | A |
| ATOM | 2242 | CB | THR | 317 | 34.132 | 6.031 | 89.783 | 1.00 | 9.66 | A |
| ATOM | 2243 | OG1 | THR | 317 | 34.077 | 6.642 | 88.496 | 1.00 | 9.89 | A |
| ATOM | 2244 | CG2 | THR | 317 | 32.870 | 5.200 | 89.994 | 1.00 | 10.70 | A |
| ATOM | 2245 | C | THR | 317 | 35.674 | 7.681 | 90.923 | 1.00 | 12.84 | A |
| ATOM | 2246 | O | THR | 317 | 36.611 | 6.965 | 91.270 | 1.00 | 13.25 | A |
| ATOM | 2247 | N | ARG | 318 | 35.852 | 8.951 | 90.575 | 1.00 | 13.06 | A |
| ATOM | 2248 | CA | ARG | 318 | 37.180 | 9.544 | 90.682 | 1.00 | 14.05 | A |
| ATOM | 2249 | CB | ARG | 318 | 37.326 | 10.780 | 89.796 | 1.00 | 15.43 | A |
| ATOM | 2250 | CG | ARG | 318 | 37.417 | 10.473 | 88.319 | 1.00 | 20.15 | A |
| ATOM | 2251 | CD | ARG | 318 | 37.526 | 11.755 | 87.527 | 1.00 | 22.93 | A |
| ATOM | 2252 | NE | ARG | 318 | 38.747 | 12.468 | 87.865 | 1.00 | 27.97 | A |
| ATOM | 2253 | CZ | ARG | 318 | 39.015 | 13.710 | 87.482 | 1.00 | 32.10 | A |
| ATOM | 2254 | NH1 | ARG | 318 | 38.138 | 14.383 | 86.747 | 1.00 | 32.47 | A |
| ATOM | 2255 | NH2 | ARG | 318 | 40.162 | 14.276 | 87.833 | 1.00 | 33.23 | A |
| ATOM | 2256 | C | ARG | 318 | 37.281 | 9.948 | 92.138 | 1.00 | 13.35 | A |
| ATOM | 2257 | O | ARG | 318 | 38.276 | 9.679 | 92.801 | 1.00 | 15.31 | A |
| ATOM | 2258 | N | ILE | 319 | 36.222 | 10.575 | 92.640 | 1.00 | 12.79 | A |
| ATOM | 2259 | CA | ILE | 319 | 36.175 | 11.012 | 94.030 | 1.00 | 11.02 | A |
| ATOM | 2260 | CB | ILE | 319 | 34.837 | 11.727 | 94.322 | 1.00 | 9.24 | A |
| ATOM | 2261 | CG2 | ILE | 319 | 34.660 | 11.958 | 95.819 | 1.00 | 4.84 | A |
| ATOM | 2262 | CG1 | ILE | 319 | 34.786 | 13.047 | 93.561 | 1.00 | 9.26 | A |
| ATOM | 2263 | CD1 | ILE | 319 | 33.431 | 13.786 | 93.692 | 1.00 | 9.14 | A |
| ATOM | 2264 | C | ILE | 319 | 36.344 | 9.833 | 95.002 | 1.00 | 12.21 | A |
| ATOM | 2265 | O | ILE | 319 | 37.127 | 9.913 | 95.950 | 1.00 | 12.33 | A |
| ATOM | 2266 | N | LEU | 320 | 35.627 | 8.739 | 94.752 | 1.00 | 10.74 | A |
| ATOM | 2267 | CA | LEU | 320 | 35.674 | 7.577 | 95.638 | 1.00 | 11.28 | A |
| ATOM | 2268 | CB | LEU | 320 | 34.240 | 7.142 | 95.965 | 1.00 | 8.50 | A |
| ATOM | 2269 | CG | LEU | 320 | 33.364 | 8.196 | 96.642 | 1.00 | 11.65 | A |
| ATOM | 2270 | CD1 | LEU | 320 | 31.909 | 7.774 | 96.550 | 1.00 | 12.32 | A |
| ATOM | 2271 | CD2 | LEU | 320 | 33.794 | 8.390 | 98.090 | 1.00 | 7.79 | A |
| ATOM | 2272 | C | LEU | 320 | 36.466 | 6.359 | 95.146 | 1.00 | 12.31 | A |
| ATOM | 2273 | O | LEU | 320 | 36.276 | 5.254 | 95.658 | 1.00 | 10.52 | A |
| ATOM | 2274 | N | GLN | 321 | 37.356 | 6.541 | 94.177 | 1.00 | 13.27 | A |
| ATOM | 2275 | CA | GLN | 321 | 38.110 | 5.401 | 93.668 | 1.00 | 16.08 | A |
| ATOM | 2276 | CB | GLN | 321 | 39.087 | 5.844 | 92.569 | 1.00 | 19.75 | A |
| ATOM | 2277 | CG | GLN | 321 | 40.196 | 6.756 | 93.006 | 1.00 | 21.68 | A |
| ATOM | 2278 | CD | GLN | 321 | 41.079 | 7.139 | 91.840 | 1.00 | 25.85 | A |
| ATOM | 2279 | OE1 | GLN | 321 | 41.622 | 6.266 | 91.152 | 1.00 | 22.98 | A |
| ATOM | 2280 | NE2 | GLN | 321 | 41.228 | 8.450 | 91.602 | 1.00 | 26.88 | A |
| ATOM | 2281 | C | GLN | 321 | 38.842 | 4.548 | 94.723 | 1.00 | 14.28 | A |
| ATOM | 2282 | O | GLN | 321 | 38.972 | 3.335 | 94.543 | 1.00 | 12.19 | A |
| ATOM | 2283 | N | ASP | 322 | 39.305 | 5.151 | 95.817 | 1.00 | 12.59 | A |
| ATOM | 2284 | CA | ASP | 322 | 39.978 | 4.351 | 96.835 | 1.00 | 14.78 | A |
| ATOM | 2285 | CB | ASP | 322 | 40.769 | 5.230 | 97.811 | 1.00 | 17.14 | A |
| ATOM | 2286 | CG | ASP | 322 | 41.787 | 4.426 | 98.620 | 1.00 | 18.36 | A |
| ATOM | 2287 | OD1 | ASP | 322 | 42.588 | 3.692 | 98.003 | 1.00 | 19.34 | A |
| ATOM | 2288 | OD2 | ASP | 322 | 41.791 | 4.521 | 99.865 | 1.00 | 19.68 | A |
| ATOM | 2289 | C | ASP | 322 | 38.988 | 3.473 | 97.609 | 1.00 | 15.66 | A |
| ATOM | 2290 | O | ASP | 322 | 39.384 | 2.598 | 98.384 | 1.00 | 17.16 | A |
| ATOM | 2291 | N | SER | 323 | 37.697 | 3.696 | 97.386 | 1.00 | 16.21 | A |
| ATOM | 2292 | CA | SER | 323 | 36.657 | 2.915 | 98.047 | 1.00 | 16.47 | A |
| ATOM | 2293 | CB | SER | 323 | 35.436 | 3.795 | 98.343 | 1.00 | 13.71 | A |
| ATOM | 2294 | OG | SER | 323 | 35.749 | 4.804 | 99.284 | 1.00 | 11.67 | A |
| ATOM | 2295 | C | SER | 323 | 36.247 | 1.735 | 97.166 | 1.00 | 18.02 | A |
| ATOM | 2296 | O | SER | 323 | 35.459 | 0.876 | 97.574 | 1.00 | 18.75 | A |
| ATOM | 2297 | N | LEU | 324 | 36.795 | 1.696 | 95.956 | 1.00 | 18.69 | A |
| ATOM | 2298 | CA | LEU | 324 | 36.495 | 0.635 | 95.009 | 1.00 | 19.76 | A |
| ATOM | 2299 | CB | LEU | 324 | 35.782 | 1.225 | 93.789 | 1.00 | 19.37 | A |
| ATOM | 2300 | CG | LEU | 324 | 34.461 | 1.920 | 94.127 | 1.00 | 19.69 | A |
| ATOM | 2301 | CD1 | LEU | 324 | 34.028 | 2.781 | 92.973 | 1.00 | 22.50 | A |
| ATOM | 2302 | CD2 | LEU | 324 | 33.394 | 0.887 | 94.449 | 1.00 | 20.33 | A |
| ATOM | 2303 | C | LEU | 324 | 37.789 | −0.045 | 94.591 | 1.00 | 21.46 | A |
| ATOM | 2304 | O | LEU | 324 | 38.427 | 0.353 | 93.618 | 1.00 | 23.00 | A |
| ATOM | 2305 | N | GLY | 325 | 38.174 | −1.074 | 95.341 | 1.00 | 22.77 | A |
| ATOM | 2306 | CA | GLY | 325 | 39.398 | −1.794 | 95.047 | 1.00 | 21.76 | A |
| ATOM | 2307 | C | GLY | 325 | 40.620 | −1.028 | 95.516 | 1.00 | 24.37 | A |
| ATOM | 2308 | O | GLY | 325 | 41.718 | −1.239 | 95.005 | 1.00 | 24.93 | A |
| ATOM | 2309 | N | GLY | 326 | 40.428 | −0.132 | 96.484 | 1.00 | 24.40 | A |
| ATOM | 2310 | CA | GLY | 326 | 41.526 | 0.663 | 97.002 | 1.00 | 24.15 | A |
| ATOM | 2311 | C | GLY | 326 | 41.897 | 0.284 | 98.424 | 1.00 | 26.42 | A |
| ATOM | 2312 | O | GLY | 326 | 41.656 | −0.840 | 98.856 | 1.00 | 25.60 | A |
| ATOM | 2313 | N | ARG | 327 | 42.470 | 1.220 | 99.168 | 1.00 | 25.86 | A |
| ATOM | 2314 | CA | ARG | 327 | 42.875 | 0.919 | 100.528 | 1.00 | 28.96 | A |
| ATOM | 2315 | CB | ARG | 327 | 44.219 | 1.593 | 100.834 | 1.00 | 32.07 | A |
| ATOM | 2316 | CG | ARG | 327 | 45.329 | 1.220 | 99.853 | 1.00 | 37.14 | A |
| ATOM | 2317 | CD | ARG | 327 | 46.714 | 1.483 | 100.432 | 1.00 | 42.74 | A |
| ATOM | 2318 | NE | ARG | 327 | 47.800 | 1.031 | 99.556 | 1.00 | 47.24 | A |
| ATOM | 2319 | CZ | ARG | 327 | 48.286 | 1.730 | 98.530 | 1.00 | 49.78 | A |
| ATOM | 2320 | NH1 | ARG | 327 | 47.787 | 2.926 | 98.237 | 1.00 | 50.77 | A |
| ATOM | 2321 | NH2 | ARG | 327 | 49.286 | 1.245 | 97.805 | 1.00 | 49.64 | A |
| ATOM | 2322 | C | ARG | 327 | 41.831 | 1.320 | 101.569 | 1.00 | 28.96 | A |
| ATOM | 2323 | O | ARG | 327 | 42.157 | 1.543 | 102.731 | 1.00 | 28.88 | A |
| ATOM | 2324 | N | THR | 328 | 40.573 | 1.401 | 101.151 | 1.00 | 27.38 | A |
| ATOM | 2325 | CA | THR | 328 | 39.499 | 1.775 | 102.064 | 1.00 | 23.77 | A |
| ATOM | 2326 | CB | THR | 328 | 38.678 | 2.944 | 101.488 | 1.00 | 24.66 | A |
| ATOM | 2327 | OG1 | THR | 328 | 39.529 | 4.088 | 101.344 | 1.00 | 25.37 | A |
| ATOM | 2328 | CG2 | THR | 328 | 37.510 | 3.292 | 102.409 | 1.00 | 23.54 | A |
| ATOM | 2329 | C | THR | 328 | 38.556 | 0.611 | 102.353 | 1.00 | 20.49 | A |
| ATOM | 2330 | O | THR | 328 | 38.287 | −0.213 | 101.480 | 1.00 | 19.13 | A |
| ATOM | 2331 | N | ARG | 329 | 38.072 | 0.532 | 103.588 | 1.00 | 17.06 | A |
| ATOM | 2332 | CA | ARG | 329 | 37.139 | −0.522 | 103.954 | 1.00 | 15.33 | A |
| ATOM | 2333 | CB | ARG | 329 | 37.126 | −0.768 | 105.465 | 1.00 | 14.79 | A |
| ATOM | 2334 | CG | ARG | 329 | 36.035 | −1.748 | 105.878 | 1.00 | 15.14 | A |
| ATOM | 2335 | CD | ARG | 329 | 35.989 | −2.023 | 107.370 | 1.00 | 17.09 | A |
| ATOM | 2336 | NE | ARG | 329 | 34.897 | −2.947 | 107.655 | 1.00 | 21.72 | A |
| ATOM | 2337 | CZ | ARG | 329 | 34.688 | −3.553 | 108.819 | 1.00 | 22.49 | A |
| ATOM | 2338 | NH1 | ARG | 329 | 35.504 | −3.343 | 109.841 | 1.00 | 20.08 | A |
| ATOM | 2339 | NH2 | ARG | 329 | 33.646 | −4.366 | 108.958 | 1.00 | 22.92 | A |
| ATOM | 2340 | C | ARG | 329 | 35.783 | −0.001 | 103.539 | 1.00 | 14.65 | A |
| ATOM | 2341 | O | ARG | 329 | 35.352 | 1.046 | 104.030 | 1.00 | 15.12 | A |
| ATOM | 2342 | N | THR | 330 | 35.107 | −0.704 | 102.640 | 1.00 | 12.06 | A |
| ATOM | 2343 | CA | THR | 330 | 33.809 | −0.226 | 102.224 | 1.00 | 14.04 | A |
| ATOM | 2344 | CB | THR | 330 | 33.837 | 0.332 | 100.782 | 1.00 | 15.17 | A |
| ATOM | 2345 | OG1 | THR | 330 | 33.694 | −0.735 | 99.847 | 1.00 | 18.22 | A |
| ATOM | 2346 | CG2 | THR | 330 | 35.147 | 1.052 | 100.513 | 1.00 | 14.36 | A |
| ATOM | 2347 | C | THR | 330 | 32.707 | −1.265 | 102.323 | 1.00 | 13.65 | A |
| ATOM | 2348 | O | THR | 330 | 32.936 | −2.459 | 102.140 | 1.00 | 13.67 | A |
| ATOM | 2349 | N | SER | 331 | 31.509 | −0.786 | 102.637 | 1.00 | 12.70 | A |
| ATOM | 2350 | CA | SER | 331 | 30.340 | −1.627 | 102.740 | 1.00 | 10.49 | A |
| ATOM | 2351 | CB | SER | 331 | 29.830 | −1.648 | 104.177 | 1.00 | 12.02 | A |
| ATOM | 2352 | OG | SER | 331 | 30.860 | −2.026 | 105.072 | 1.00 | 18.36 | A |
| ATOM | 2353 | C | SER | 331 | 29.259 | −1.044 | 101.830 | 1.00 | 10.83 | A |
| ATOM | 2354 | O | SER | 331 | 29.235 | 0.160 | 101.555 | 1.00 | 8.62 | A |
| ATOM | 2355 | N | ILE | 332 | 28.376 | −1.906 | 101.349 | 1.00 | 9.52 | A |
| ATOM | 2356 | CA | ILE | 332 | 27.288 | −1.457 | 100.511 | 1.00 | 9.50 | A |
| ATOM | 2357 | CB | ILE | 332 | 27.374 | −2.038 | 99.089 | 1.00 | 10.70 | A |
| ATOM | 2358 | CG2 | ILE | 332 | 26.143 | −1.622 | 98.287 | 1.00 | 8.05 | A |
| ATOM | 2359 | CG1 | ILE | 332 | 28.650 | −1.560 | 98.394 | 1.00 | 9.17 | A |
| ATOM | 2360 | CD1 | ILE | 332 | 28.773 | −2.094 | 96.975 | 1.00 | 5.23 | A |
| ATOM | 2361 | C | ILE | 332 | 25.993 | −1.939 | 101.138 | 1.00 | 9.51 | A |
| ATOM | 2362 | O | ILE | 332 | 25.843 | −3.127 | 101.413 | 1.00 | 9.19 | A |
| ATOM | 2363 | N | ILE | 333 | 25.074 | −1.015 | 101.391 | 1.00 | 9.81 | A |
| ATOM | 2364 | CA | ILE | 333 | 23.773 | −1.376 | 101.942 | 1.00 | 8.92 | A |
| ATOM | 2365 | CB | ILE | 333 | 23.335 | −0.444 | 103.103 | 1.00 | 8.82 | A |
| ATOM | 2366 | CG2 | ILE | 333 | 21.967 | −0.863 | 103.614 | 1.00 | 7.93 | A |
| ATOM | 2367 | CG1 | ILE | 333 | 24.316 | −0.548 | 104.272 | 1.00 | 6.76 | A |
| ATOM | 2368 | CD1 | ILE | 333 | 24.028 | 0.448 | 105.387 | 1.00 | 2.97 | A |
| ATOM | 2369 | C | ILE | 333 | 22.777 | −1.240 | 100.797 | 1.00 | 9.34 | A |
| ATOM | 2370 | O | ILE | 333 | 22.483 | −0.132 | 100.347 | 1.00 | 6.58 | A |
| ATOM | 2371 | N | ALA | 334 | 22.294 | −2.376 | 100.303 | 1.00 | 9.13 | A |
| ATOM | 2372 | CA | ALA | 334 | 21.325 | −2.370 | 99.215 | 1.00 | 8.43 | A |
| ATOM | 2373 | CB | ALA | 334 | 21.543 | −3.582 | 98.318 | 1.00 | 6.36 | A |
| ATOM | 2374 | C | ALA | 334 | 19.903 | −2.381 | 99.807 | 1.00 | 8.65 | A |
| ATOM | 2375 | O | ALA | 334 | 19.555 | −3.232 | 100.634 | 1.00 | 6.98 | A |
| ATOM | 2376 | N | THR | 335 | 19.089 | −1.419 | 99.398 | 1.00 | 8.61 | A |
| ATOM | 2377 | CA | THR | 335 | 17.727 | −1.334 | 99.899 | 1.00 | 8.77 | A |
| ATOM | 2378 | CB | THR | 335 | 17.375 | 0.092 | 100.290 | 1.00 | 7.57 | A |
| ATOM | 2379 | OG1 | THR | 335 | 17.538 | 0.949 | 99.157 | 1.00 | 8.21 | A |
| ATOM | 2380 | CG2 | THR | 335 | 18.276 | 0.552 | 101.398 | 1.00 | 7.82 | A |
| ATOM | 2381 | C | THR | 335 | 16.729 | −1.820 | 98.863 | 1.00 | 8.70 | A |
| ATOM | 2382 | O | THR | 335 | 16.855 | −1.530 | 97.671 | 1.00 | 8.21 | A |
| ATOM | 2383 | N | ILE | 336 | 15.735 | −2.560 | 99.338 | 1.00 | 8.74 | A |
| ATOM | 2384 | CA | ILE | 336 | 14.717 | −3.124 | 98.469 | 1.00 | 10.87 | A |
| ATOM | 2385 | CB | ILE | 336 | 14.998 | −4.613 | 98.216 | 1.00 | 10.46 | A |
| ATOM | 2386 | CG2 | ILE | 336 | 16.353 | −4.769 | 97.532 | 1.00 | 8.62 | A |
| ATOM | 2387 | CG1 | ILE | 336 | 14.943 | −5.379 | 99.543 | 1.00 | 10.60 | A |
| ATOM | 2388 | CD1 | ILE | 336 | 14.993 | −6.921 | 99.386 | 1.00 | 10.91 | A |
| ATOM | 2389 | C | ILE | 336 | 13.291 | −2.995 | 99.004 | 1.00 | 12.03 | A |
| ATOM | 2390 | O | ILE | 336 | 13.069 | −2.844 | 100.204 | 1.00 | 12.49 | A |
| ATOM | 2391 | N | SER | 337 | 12.331 | −3.056 | 98.089 | 1.00 | 13.93 | A |
| ATOM | 2392 | CA | SER | 337 | 10.918 | −2.969 | 98.426 | 1.00 | 13.83 | A |
| ATOM | 2393 | CB | SER | 337 | 10.180 | −2.154 | 97.359 | 1.00 | 14.05 | A |
| ATOM | 2394 | OG | SER | 337 | 8.790 | −2.436 | 97.350 | 1.00 | 13.32 | A |
| ATOM | 2395 | C | SER | 337 | 10.371 | −4.386 | 98.464 | 1.00 | 14.60 | A |
| ATOM | 2396 | O | SER | 337 | 10.829 | −5.250 | 97.717 | 1.00 | 14.95 | A |
| ATOM | 2397 | N | PRO | 338 | 9.398 | −4.652 | 99.350 | 1.00 | 15.93 | A |
| ATOM | 2398 | CD | PRO | 338 | 8.967 | −3.811 | 100.483 | 1.00 | 16.39 | A |
| ATOM | 2399 | CA | PRO | 338 | 8.809 | −5.990 | 99.451 | 1.00 | 15.42 | A |
| ATOM | 2400 | CB | PRO | 338 | 8.461 | −6.088 | 100.921 | 1.00 | 15.52 | A |
| ATOM | 2401 | CG | PRO | 338 | 7.930 | −4.705 | 101.176 | 1.00 | 17.59 | A |
| ATOM | 2402 | C | PRO | 338 | 7.564 | −6.138 | 98.576 | 1.00 | 15.52 | A |
| ATOM | 2403 | O | PRO | 338 | 6.929 | −7.185 | 98.571 | 1.00 | 17.10 | A |
| ATOM | 2404 | N | ALA | 339 | 7.212 | −5.091 | 97.841 | 1.00 | 15.73 | A |
| ATOM | 2405 | CA | ALA | 339 | 6.023 | −5.122 | 96.989 | 1.00 | 17.08 | A |
| ATOM | 2406 | CB | ALA | 339 | 5.494 | −3.699 | 96.765 | 1.00 | 13.90 | A |
| ATOM | 2407 | C | ALA | 339 | 6.255 | −5.793 | 95.647 | 1.00 | 17.79 | A |
| ATOM | 2408 | O | ALA | 339 | 7.290 | −5.586 | 95.010 | 1.00 | 18.27 | A |
| ATOM | 2409 | N | SER | 340 | 5.270 | −6.575 | 95.210 | 1.00 | 19.26 | A |
| ATOM | 2410 | CA | SER | 340 | 5.339 | −7.280 | 93.933 | 1.00 | 20.19 | A |
| ATOM | 2411 | CB | SER | 340 | 4.088 | −8.151 | 93.741 | 1.00 | 21.56 | A |
| ATOM | 2412 | OG | SER | 340 | 2.909 | −7.370 | 93.812 | 1.00 | 24.50 | A |
| ATOM | 2413 | C | SER | 340 | 5.495 | −6.340 | 92.736 | 1.00 | 18.83 | A |
| ATOM | 2414 | O | SER | 340 | 5.977 | −6.755 | 91.687 | 1.00 | 17.98 | A |
| ATOM | 2415 | N | LEU | 341 | 5.083 | −5.084 | 92.883 | 1.00 | 19.49 | A |
| ATOM | 2416 | CA | LEU | 341 | 5.212 | −4.114 | 91.793 | 1.00 | 21.42 | A |
| ATOM | 2417 | CB | LEU | 341 | 4.539 | −2.787 | 92.159 | 1.00 | 24.24 | A |
| ATOM | 2418 | CG | LEU | 341 | 3.056 | −2.763 | 92.528 | 1.00 | 30.57 | A |
| ATOM | 2419 | CD1 | LEU | 341 | 2.838 | −3.310 | 93.952 | 1.00 | 30.86 | A |
| ATOM | 2420 | CD2 | LEU | 341 | 2.563 | −1.325 | 92.435 | 1.00 | 32.23 | A |
| ATOM | 2421 | C | LEU | 341 | 6.678 | −3.821 | 91.452 | 1.00 | 20.58 | A |
| ATOM | 2422 | O | LEU | 341 | 7.017 | −3.528 | 90.308 | 1.00 | 20.62 | A |
| ATOM | 2423 | N | ASN | 342 | 7.544 | −3.905 | 92.455 | 1.00 | 19.46 | A |
| ATOM | 2424 | CA | ASN | 342 | 8.958 | −3.620 | 92.267 | 1.00 | 18.47 | A |
| ATOM | 2425 | CB | ASN | 342 | 9.471 | −2.863 | 93.485 | 1.00 | 17.34 | A |
| ATOM | 2426 | CG | ASN | 342 | 8.662 | −1.618 | 93.763 | 1.00 | 16.86 | A |
| ATOM | 2427 | OD1 | ASN | 342 | 8.564 | −0.730 | 92.916 | 1.00 | 18.67 | A |
| ATOM | 2428 | ND2 | ASN | 342 | 8.070 | −1.546 | 94.944 | 1.00 | 15.28 | A |
| ATOM | 2429 | C | ASN | 342 | 9.795 | −4.871 | 92.041 | 1.00 | 18.85 | A |
| ATOM | 2430 | O | ASN | 342 | 10.988 | −4.893 | 92.351 | 1.00 | 17.91 | A |
| ATOM | 2431 | N | LEU | 343 | 9.170 | −5.908 | 91.493 | 1.00 | 17.20 | A |
| ATOM | 2432 | CA | LEU | 343 | 9.863 | −7.163 | 91.252 | 1.00 | 17.19 | A |
| ATOM | 2433 | CB | LEU | 343 | 8.917 | −8.179 | 90.596 | 1.00 | 13.78 | A |
| ATOM | 2434 | CG | LEU | 343 | 9.593 | −9.472 | 90.107 | 1.00 | 14.61 | A |
| ATOM | 2435 | CD1 | LEU | 343 | 10.343 | −10.143 | 91.269 | 1.00 | 10.55 | A |
| ATOM | 2436 | CD2 | LEU | 343 | 8.554 | −10.415 | 89.499 | 1.00 | 13.10 | A |
| ATOM | 2437 | C | LEU | 343 | 11.115 | −7.020 | 90.399 | 1.00 | 17.48 | A |
| ATOM | 2438 | O | LEU | 343 | 12.211 | −7.377 | 90.829 | 1.00 | 17.34 | A |
| ATOM | 2439 | N | GLU | 344 | 10.946 | −6.514 | 89.184 | 1.00 | 19.72 | A |
| ATOM | 2440 | CA | GLU | 344 | 12.063 | −6.358 | 88.263 | 1.00 | 20.96 | A |
| ATOM | 2441 | CB | GLU | 344 | 11.598 | −5.684 | 86.969 | 1.00 | 24.20 | A |
| ATOM | 2442 | CG | GLU | 344 | 12.675 | −5.635 | 85.887 | 1.00 | 32.62 | A |
| ATOM | 2443 | CD | GLU | 344 | 12.213 | −4.959 | 84.599 | 1.00 | 38.13 | A |
| ATOM | 2444 | OE1 | GLU | 344 | 12.908 | −5.115 | 83.566 | 1.00 | 40.01 | A |
| ATOM | 2445 | OE2 | GLU | 344 | 11.165 | −4.270 | 84.617 | 1.00 | 41.47 | A |
| ATOM | 2446 | C | GLU | 344 | 13.208 | −5.561 | 88.883 | 1.00 | 20.19 | A |
| ATOM | 2447 | O | GLU | 344 | 14.371 | −5.957 | 88.791 | 1.00 | 20.32 | A |
| ATOM | 2448 | N | GLU | 345 | 12.883 | −4.441 | 89.518 | 1.00 | 17.74 | A |
| ATOM | 2449 | CA | GLU | 345 | 13.909 | −3.615 | 90.130 | 1.00 | 18.84 | A |
| ATOM | 2450 | CB | GLU | 345 | 13.335 | −2.240 | 90.496 | 1.00 | 21.25 | A |
| ATOM | 2451 | CG | GLU | 345 | 13.076 | −1.356 | 89.281 | 1.00 | 24.52 | A |
| ATOM | 2452 | CD | GLU | 345 | 14.348 | −1.036 | 88.492 | 1.00 | 27.03 | A |
| ATOM | 2453 | OE1 | GLU | 345 | 14.232 | −0.592 | 87.325 | 1.00 | 29.83 | A |
| ATOM | 2454 | OE2 | GLU | 345 | 15.462 | −1.216 | 89.036 | 1.00 | 27.61 | A |
| ATOM | 2455 | C | GLU | 345 | 14.555 | −4.270 | 91.346 | 1.00 | 16.79 | A |
| ATOM | 2456 | O | GLU | 345 | 15.762 | −4.143 | 91.554 | 1.00 | 17.33 | A |
| ATOM | 2457 | N | THR | 346 | 13.760 | −4.978 | 92.140 | 1.00 | 14.42 | A |
| ATOM | 2458 | CA | THR | 346 | 14.286 | −5.649 | 93.316 | 1.00 | 14.40 | A |
| ATOM | 2459 | CB | THR | 346 | 13.160 | −6.304 | 94.138 | 1.00 | 15.55 | A |
| ATOM | 2460 | OG1 | THR | 346 | 12.399 | −5.285 | 94.801 | 1.00 | 13.04 | A |
| ATOM | 2461 | CG2 | THR | 346 | 13.735 | −7.255 | 95.171 | 1.00 | 15.14 | A |
| ATOM | 2462 | C | THR | 346 | 15.302 | −6.705 | 92.896 | 1.00 | 14.50 | A |
| ATOM | 2463 | O | THR | 346 | 16.294 | −6.922 | 93.590 | 1.00 | 13.63 | A |
| ATOM | 2464 | N | LEU | 347 | 15.061 | −7.362 | 91.763 | 1.00 | 14.51 | A |
| ATOM | 2465 | CA | LEU | 347 | 16.005 | −8.357 | 91.269 | 1.00 | 15.49 | A |
| ATOM | 2466 | CB | LEU | 347 | 15.369 | −9.222 | 90.167 | 1.00 | 15.24 | A |
| ATOM | 2467 | CG | LEU | 347 | 14.220 | −10.158 | 90.571 | 1.00 | 15.51 | A |
| ATOM | 2468 | CD1 | LEU | 347 | 13.712 | −10.902 | 89.351 | 1.00 | 11.90 | A |
| ATOM | 2469 | CD2 | LEU | 347 | 14.687 | −11.142 | 91.627 | 1.00 | 13.17 | A |
| ATOM | 2470 | C | LEU | 347 | 17.267 | −7.666 | 90.734 | 1.00 | 16.52 | A |
| ATOM | 2471 | O | LEU | 347 | 18.376 | −8.175 | 90.908 | 1.00 | 18.79 | A |
| ATOM | 2472 | N | SER | 348 | 17.111 | −6.513 | 90.088 | 1.00 | 15.74 | A |
| ATOM | 2473 | CA | SER | 348 | 18.274 | −5.795 | 89.567 | 1.00 | 16.97 | A |
| ATOM | 2474 | CB | SER | 348 | 17.857 | −4.502 | 88.872 | 1.00 | 17.03 | A |
| ATOM | 2475 | OG | SER | 348 | 17.008 | −4.785 | 87.780 | 1.00 | 23.78 | A |
| ATOM | 2476 | C | SER | 348 | 19.199 | −5.438 | 90.712 | 1.00 | 16.29 | A |
| ATOM | 2477 | O | SER | 348 | 20.415 | −5.668 | 90.655 | 1.00 | 17.03 | A |
| ATOM | 2478 | N | THR | 349 | 18.603 | −4.864 | 91.751 | 1.00 | 13.43 | A |
| ATOM | 2479 | CA | THR | 349 | 19.341 | −4.452 | 92.925 | 1.00 | 12.53 | A |
| ATOM | 2480 | CB | THR | 349 | 18.400 | −3.808 | 93.953 | 1.00 | 11.53 | A |
| ATOM | 2481 | OG1 | THR | 349 | 17.883 | −2.583 | 93.416 | 1.00 | 12.14 | A |
| ATOM | 2482 | CG2 | THR | 349 | 19.143 | −3.512 | 95.243 | 1.00 | 8.21 | A |
| ATOM | 2483 | C | THR | 349 | 20.074 | −5.624 | 93.563 | 1.00 | 12.73 | A |
| ATOM | 2484 | O | THR | 349 | 21.292 | −5.590 | 93.732 | 1.00 | 10.74 | A |
| ATOM | 2485 | N | LEU | 350 | 19.325 | −6.660 | 93.916 | 1.00 | 14.33 | A |
| ATOM | 2486 | CA | LEU | 350 | 19.923 | −7.830 | 94.532 | 1.00 | 16.65 | A |
| ATOM | 2487 | CB | LEU | 350 | 18.855 | −8.892 | 94.803 | 1.00 | 14.51 | A |
| ATOM | 2488 | CG | LEU | 350 | 17.916 | −8.537 | 95.960 | 1.00 | 13.75 | A |
| ATOM | 2489 | CD1 | LEU | 350 | 16.780 | −9.516 | 96.035 | 1.00 | 10.80 | A |
| ATOM | 2490 | CD2 | LEU | 350 | 18.703 | −8.526 | 97.258 | 1.00 | 15.25 | A |
| ATOM | 2491 | C | LEU | 350 | 21.033 | −8.400 | 93.660 | 1.00 | 17.62 | A |
| ATOM | 2492 | O | LEU | 350 | 22.116 | −8.695 | 94.148 | 1.00 | 19.69 | A |
| ATOM | 2493 | N | GLU | 351 | 20.774 | −8.540 | 92.368 | 1.00 | 18.77 | A |
| ATOM | 2494 | CA | GLU | 351 | 21.783 | −9.078 | 91.466 | 1.00 | 20.26 | A |
| ATOM | 2495 | CB | GLU | 351 | 21.203 | −9.215 | 90.061 | 1.00 | 23.16 | A |
| ATOM | 2496 | CG | GLU | 351 | 21.961 | −10.194 | 89.186 | 1.00 | 31.07 | A |
| ATOM | 2497 | CD | GLU | 351 | 21.645 | −11.652 | 89.508 | 1.00 | 35.15 | A |
| ATOM | 2498 | OE1 | GLU | 351 | 22.421 | −12.531 | 89.070 | 1.00 | 37.94 | A |
| ATOM | 2499 | OE2 | GLU | 351 | 20.621 | −11.921 | 90.180 | 1.00 | 35.11 | A |
| ATOM | 2500 | C | GLU | 351 | 23.030 | −8.181 | 91.440 | 1.00 | 18.73 | A |
| ATOM | 2501 | O | GLU | 351 | 24.163 | −8.662 | 91.407 | 1.00 | 18.86 | A |
| ATOM | 2502 | N | TYR | 352 | 22.810 | −6.873 | 91.463 | 1.00 | 18.82 | A |
| ATOM | 2503 | CA | TYR | 352 | 23.893 | −5.898 | 91.443 | 1.00 | 16.90 | A |
| ATOM | 2504 | CB | TYR | 352 | 23.304 | −4.500 | 91.261 | 1.00 | 17.28 | A |
| ATOM | 2505 | CG | TYR | 352 | 24.306 | −3.374 | 91.118 | 1.00 | 15.30 | A |
| ATOM | 2506 | CD1 | TYR | 352 | 24.940 | −2.833 | 92.227 | 1.00 | 12.89 | A |
| ATOM | 2507 | CE1 | TYR | 352 | 25.779 | −1.740 | 92.100 | 1.00 | 15.82 | A |
| ATOM | 2508 | CD2 | TYR | 352 | 24.550 | −2.798 | 89.869 | 1.00 | 15.34 | A |
| ATOM | 2509 | CE2 | TYR | 352 | 25.382 | −1.712 | 89.731 | 1.00 | 14.65 | A |
| ATOM | 2510 | CZ | TYR | 352 | 25.989 | −1.180 | 90.848 | 1.00 | 15.26 | A |
| ATOM | 2511 | OH | TYR | 352 | 26.767 | −0.050 | 90.715 | 1.00 | 17.76 | A |
| ATOM | 2512 | C | TYR | 352 | 24.688 | −5.973 | 92.733 | 1.00 | 16.43 | A |
| ATOM | 2513 | O | TYR | 352 | 25.917 | −5.964 | 92.715 | 1.00 | 17.51 | A |
| ATOM | 2514 | N | ALA | 353 | 23.989 | −6.065 | 93.855 | 1.00 | 15.81 | A |
| ATOM | 2515 | CA | ALA | 353 | 24.658 | −6.137 | 95.145 | 1.00 | 16.65 | A |
| ATOM | 2516 | CB | ALA | 353 | 23.646 | −5.931 | 96.269 | 1.00 | 15.23 | A |
| ATOM | 2517 | C | ALA | 353 | 25.405 | −7.458 | 95.350 | 1.00 | 17.40 | A |
| ATOM | 2518 | O | ALA | 353 | 26.412 | −7.497 | 96.050 | 1.00 | 18.96 | A |
| ATOM | 2519 | N | HIS | 354 | 24.916 | −8.535 | 94.744 | 1.00 | 18.26 | A |
| ATOM | 2520 | CA | HIS | 354 | 25.555 | −9.838 | 94.883 | 1.00 | 19.76 | A |
| ATOM | 2521 | CB | HIS | 354 | 24.676 | −10.932 | 94.266 | 1.00 | 19.50 | A |
| ATOM | 2522 | CG | HIS | 354 | 25.143 | −12.324 | 94.566 | 1.00 | 21.21 | A |
| ATOM | 2523 | CD2 | HIS | 354 | 25.758 | −13.246 | 93.786 | 1.00 | 20.11 | A |
| ATOM | 2524 | ND1 | HIS | 354 | 25.032 | −12.894 | 95.817 | 1.00 | 20.61 | A |
| ATOM | 2525 | CE1 | HIS | 354 | 25.561 | −14.105 | 95.796 | 1.00 | 20.62 | A |
| ATOM | 2526 | NE2 | HIS | 354 | 26.009 | −14.342 | 94.576 | 1.00 | 20.83 | A |
| ATOM | 2527 | C | HIS | 354 | 26.936 | −9.842 | 94.224 | 1.00 | 21.08 | A |
| ATOM | 2528 | O | HIS | 354 | 27.903 | −10.313 | 94.816 | 1.00 | 22.05 | A |
| ATOM | 2529 | N | ARG | 355 | 27.027 | −9.314 | 93.004 | 1.00 | 22.49 | A |
| ATOM | 2530 | CA | ARG | 355 | 28.308 | −9.256 | 92.292 | 1.00 | 24.62 | A |
| ATOM | 2531 | CB | ARG | 355 | 28.153 | −8.619 | 90.905 | 1.00 | 25.83 | A |
| ATOM | 2532 | CG | ARG | 355 | 27.358 | −9.413 | 89.894 | 1.00 | 29.38 | A |
| ATOM | 2533 | CD | ARG | 355 | 27.482 | −8.762 | 88.535 | 1.00 | 32.38 | A |
| ATOM | 2534 | NE | ARG | 355 | 27.233 | −7.326 | 88.622 | 1.00 | 37.22 | A |
| ATOM | 2535 | CZ | ARG | 355 | 27.902 | −6.412 | 87.924 | 1.00 | 40.93 | A |
| ATOM | 2536 | NH1 | ARG | 355 | 28.860 | −6.797 | 87.087 | 1.00 | 41.58 | A |
| ATOM | 2537 | NH2 | ARG | 355 | 27.624 | −5.117 | 88.066 | 1.00 | 39.72 | A |
| ATOM | 2538 | C | ARG | 355 | 29.352 | −8.447 | 93.054 | 1.00 | 24.34 | A |
| ATOM | 2539 | O | ARG | 355 | 30.523 | −8.821 | 93.098 | 1.00 | 25.69 | A |
| ATOM | 2540 | N | ALA | 356 | 28.923 | −7.332 | 93.640 | 1.00 | 23.36 | A |
| ATOM | 2541 | CA | ALA | 356 | 29.814 | −6.447 | 94.387 | 1.00 | 22.82 | A |
| ATOM | 2542 | CB | ALA | 356 | 29.016 | −5.295 | 94.985 | 1.00 | 20.20 | A |
| ATOM | 2543 | C | ALA | 356 | 30.603 | −7.161 | 95.484 | 1.00 | 23.12 | A |
| ATOM | 2544 | O | ALA | 356 | 31.708 | −6.751 | 95.820 | 1.00 | 20.69 | A |
| ATOM | 2545 | N | LYS | 357 | 30.030 | −8.222 | 96.047 | 1.00 | 24.95 | A |
| ATOM | 2546 | CA | LYS | 357 | 30.695 | −8.981 | 97.111 | 1.00 | 26.72 | A |
| ATOM | 2547 | CB | LYS | 357 | 29.849 | −10.195 | 97.497 | 1.00 | 25.95 | A |
| ATOM | 2548 | CG | LYS | 357 | 28.570 | −9.854 | 98.232 | 1.00 | 27.20 | A |
| ATOM | 2549 | CD | LYS | 357 | 27.647 | −11.052 | 98.293 | 1.00 | 28.41 | A |
| ATOM | 2550 | CE | LYS | 357 | 28.288 | −12.220 | 99.024 | 1.00 | 29.67 | A |
| ATOM | 2551 | NZ | LYS | 357 | 27.537 | −13.483 | 98.790 | 1.00 | 30.65 | A |
| ATOM | 2552 | C | LYS | 357 | 32.099 | −9.453 | 96.733 | 1.00 | 27.68 | A |
| ATOM | 2553 | O | LYS | 357 | 32.968 | −9.595 | 97.601 | 1.00 | 26.10 | A |
| ATOM | 2554 | N | ASN | 358 | 32.312 | −9.691 | 95.438 | 1.00 | 28.56 | A |
| ATOM | 2555 | CA | ASN | 358 | 33.591 | −10.177 | 94.925 | 1.00 | 28.98 | A |
| ATOM | 2556 | CB | ASN | 358 | 33.363 | −10.897 | 93.597 | 1.00 | 31.13 | A |
| ATOM | 2557 | CG | ASN | 358 | 32.415 | −12.071 | 93.735 | 1.00 | 34.60 | A |
| ATOM | 2558 | OD1 | ASN | 358 | 32.743 | −13.071 | 94.375 | 1.00 | 37.20 | A |
| ATOM | 2559 | ND2 | ASN | 358 | 31.226 | −11.952 | 93.145 | 1.00 | 33.97 | A |
| ATOM | 2560 | C | ASN | 358 | 34.676 | −9.118 | 94.751 | 1.00 | 27.98 | A |
| ATOM | 2561 | O | ASN | 358 | 35.784 | −9.426 | 94.316 | 1.00 | 28.50 | A |
| ATOM | 2562 | N | ILE | 359 | 34.364 | −7.871 | 95.079 | 1.00 | 25.92 | A |
| ATOM | 2563 | CA | ILE | 359 | 35.350 | −6.811 | 94.957 | 1.00 | 24.09 | A |
| ATOM | 2564 | CB | ILE | 359 | 34.673 | −5.429 | 94.910 | 1.00 | 21.25 | A |
| ATOM | 2565 | CG2 | ILE | 359 | 35.727 | −4.329 | 94.867 | 1.00 | 19.17 | A |
| ATOM | 2566 | CG1 | ILE | 359 | 33.748 | −5.367 | 93.689 | 1.00 | 19.08 | A |
| ATOM | 2567 | CD1 | ILE | 359 | 32.909 | −4.109 | 93.597 | 1.00 | 18.25 | A |
| ATOM | 2568 | C | ILE | 359 | 36.290 | −6.906 | 96.155 | 1.00 | 25.26 | A |
| ATOM | 2569 | O | ILE | 359 | 35.847 | −7.076 | 97.290 | 1.00 | 23.96 | A |
| ATOM | 2570 | N | LEU | 360 | 37.588 | −6.817 | 95.897 | 1.00 | 27.58 | A |
| ATOM | 2571 | CA | LEU | 360 | 38.578 | −6.917 | 96.963 | 1.00 | 32.07 | A |
| ATOM | 2572 | CB | LEU | 360 | 39.478 | −8.137 | 96.722 | 1.00 | 34.40 | A |
| ATOM | 2573 | CG | LEU | 360 | 40.711 | −8.333 | 97.613 | 1.00 | 36.57 | A |
| ATOM | 2574 | CD1 | LEU | 360 | 40.309 | −8.930 | 98.961 | 1.00 | 37.87 | A |
| ATOM | 2575 | CD2 | LEU | 360 | 41.687 | −9.265 | 96.913 | 1.00 | 38.48 | A |
| ATOM | 2576 | C | LEU | 360 | 39.438 | −5.665 | 97.033 | 1.00 | 33.54 | A |
| ATOM | 2577 | O | LEU | 360 | 39.905 | −5.174 | 96.008 | 1.00 | 32.97 | A |
| ATOM | 2578 | N | ASN | 361 | 39.635 | −5.132 | 98.234 | 1.00 | 35.62 | A |
| ATOM | 2579 | CA | ASN | 361 | 40.485 | −3.962 | 98.372 | 1.00 | 39.86 | A |
| ATOM | 2580 | CB | ASN | 361 | 39.649 | −2.672 | 98.395 | 1.00 | 41.32 | A |
| ATOM | 2581 | CG | ASN | 361 | 38.490 | −2.732 | 99.345 | 1.00 | 42.28 | A |
| ATOM | 2582 | OD1 | ASN | 361 | 37.523 | −1.985 | 99.203 | 1.00 | 42.60 | A |
| ATOM | 2583 | ND2 | ASN | 361 | 38.578 | −3.609 | 100.330 | 1.00 | 45.41 | A |
| ATOM | 2584 | C | ASN | 361 | 41.439 | −4.056 | 99.565 | 1.00 | 41.68 | A |
| ATOM | 2585 | O | ASN | 361 | 41.180 | −4.768 | 100.532 | 1.00 | 41.90 | A |
| ATOM | 2586 | N | LYS | 362 | 42.560 | −3.348 | 99.446 | 1.00 | 44.89 | A |
| ATOM | 2587 | CA | LYS | 362 | 43.643 | −3.321 | 100.432 | 1.00 | 46.74 | A |
| ATOM | 2588 | CB | LYS | 362 | 43.106 | −3.372 | 101.870 | 1.00 | 45.91 | A |
| ATOM | 2589 | CG | LYS | 362 | 42.518 | −2.057 | 102.353 | 1.00 | 44.95 | A |
| ATOM | 2590 | CD | LYS | 362 | 42.184 | −2.089 | 103.841 | 1.00 | 44.77 | A |
| ATOM | 2591 | CE | LYS | 362 | 43.444 | −2.056 | 104.701 | 1.00 | 44.68 | A |
| ATOM | 2592 | NZ | LYS | 362 | 44.224 | −0.795 | 104.523 | 1.00 | 44.09 | A |
| ATOM | 2593 | C | LYS | 362 | 44.576 | −4.504 | 100.173 | 1.00 | 48.88 | A |
| ATOM | 2594 | O | LYS | 362 | 44.928 | −5.219 | 101.141 | 1.00 | 50.91 | A |
| ATOM | 2595 | OXT | LYS | 362 | 44.955 | −4.700 | 98.992 | 1.00 | 49.21 | A |
| ATOM | 2596 | MG | MG | 603 | 16.038 | 9.381 | 98.154 | 1.00 | 22.45 | |
| ATOM | 2597 | PB | ADP | 601 | 14.871 | 6.512 | 98.896 | 1.00 | 9.83 | ADP |
| ATOM | 2598 | O1B | ADP | 601 | 14.389 | 7.073 | 97.604 | 1.00 | 11.43 | ADP |
| ATOM | 2599 | O2B | ADP | 601 | 15.417 | 5.029 | 98.682 | 1.00 | 12.43 | ADP |
| ATOM | 2600 | O3B | ADP | 601 | 15.921 | 7.374 | 99.491 | 1.00 | 9.54 | ADP |
| ATOM | 2601 | PA | ADP | 601 | 13.343 | 7.143 | 101.254 | 1.00 | 13.34 | ADP |
| ATOM | 2602 | O1A | ADP | 601 | 14.336 | 6.832 | 102.280 | 1.00 | 14.02 | ADP |
| ATOM | 2603 | O2A | ADP | 601 | 13.336 | 8.581 | 101.013 | 1.00 | 12.22 | ADP |
| ATOM | 2604 | O3A | ADP | 601 | 13.676 | 6.373 | 99.912 | 1.00 | 11.56 | ADP |
| ATOM | 2605 | O5* | ADP | 601 | 11.879 | 6.778 | 101.742 | 1.00 | 16.31 | ADP |
| ATOM | 2606 | C5* | ADP | 601 | 10.894 | 5.934 | 101.155 | 1.00 | 16.15 | ADP |
| ATOM | 2607 | C4* | ADP | 601 | 9.662 | 5.974 | 102.132 | 1.00 | 18.96 | ADP |
| ATOM | 2608 | O4* | ADP | 601 | 9.712 | 4.734 | 102.849 | 1.00 | 19.62 | ADP |
| ATOM | 2609 | C3* | ADP | 601 | 9.700 | 7.065 | 103.229 | 1.00 | 18.60 | ADP |
| ATOM | 2610 | O3* | ADP | 601 | 8.406 | 7.650 | 103.431 | 1.00 | 22.72 | ADP |
| ATOM | 2611 | C2* | ADP | 601 | 10.188 | 6.391 | 104.496 | 1.00 | 19.66 | ADP |
| ATOM | 2612 | O2* | ADP | 601 | 9.655 | 6.994 | 105.672 | 1.00 | 21.78 | ADP |
| ATOM | 2613 | C1* | ADP | 601 | 9.788 | 4.947 | 104.281 | 1.00 | 19.08 | ADP |
| ATOM | 2614 | N9 | ADP | 601 | 10.778 | 3.943 | 104.795 | 1.00 | 19.36 | ADP |
| ATOM | 2615 | C8 | ADP | 601 | 11.895 | 3.536 | 104.137 | 1.00 | 19.33 | ADP |
| ATOM | 2616 | N7 | ADP | 601 | 12.535 | 2.641 | 104.859 | 1.00 | 19.29 | ADP |
| ATOM | 2617 | C5 | ADP | 601 | 11.874 | 2.450 | 105.961 | 1.00 | 20.60 | ADP |
| ATOM | 2618 | C6 | ADP | 601 | 12.043 | 1.649 | 107.091 | 1.00 | 20.38 | ADP |
| ATOM | 2619 | N6 | ADP | 601 | 13.085 | 0.825 | 107.178 | 1.00 | 20.28 | ADP |
| ATOM | 2620 | N1 | ADP | 601 | 11.118 | 1.701 | 108.120 | 1.00 | 22.79 | ADP |
| ATOM | 2621 | C2 | ADP | 601 | 10.028 | 2.524 | 108.081 | 1.00 | 22.78 | ADP |
| ATOM | 2622 | N3 | ADP | 601 | 9.854 | 3.302 | 106.988 | 1.00 | 20.98 | ADP |
| ATOM | 2623 | C4 | ADP | 601 | 10.736 | 3.301 | 105.936 | 1.00 | 20.39 | ADP |
| ATOM | 2859 | C1 | 5-2b | 2 | 19.000 | 14.175 | 112.199 | 1.00 | 28.18 | 5-2b |
| ATOM | 2860 | C2 | 5-2b | 2 | 18.061 | 13.539 | 111.340 | 1.00 | 32.48 | 5-2b |
| ATOM | 2861 | C3 | 5-2b | 2 | 17.078 | 12.651 | 111.895 | 1.00 | 28.56 | 5-2b |
| ATOM | 2862 | C4 | 5-2b | 2 | 17.088 | 12.427 | 113.305 | 1.00 | 27.05 | 5-2b |
| ATOM | 2863 | C5 | 5-2b | 2 | 18.039 | 13.044 | 114.157 | 1.00 | 26.16 | 5-2b |
| ATOM | 2864 | C6 | 5-2b | 2 | 19.015 | 13.950 | 113.622 | 1.00 | 28.62 | 5-2b |
| ATOM | 2865 | C7 | 5-2b | 2 | 18.128 | 13.723 | 109.878 | 1.00 | 39.58 | 5-2b |
| ATOM | 2866 | N8 | 5-2b | 2 | 19.295 | 13.211 | 109.173 | 1.00 | 34.03 | 5-2b |
| ATOM | 2867 | C9 | 5-2b | 2 | 20.221 | 14.007 | 108.603 | 1.00 | 31.92 | 5-2b |
| ATOM | 2868 | N10 | 5-2b | 2 | 19.947 | 15.297 | 108.469 | 1.00 | 36.78 | 5-2b |
| ATOM | 2869 | C11 | 5-2b | 2 | 18.661 | 15.862 | 108.801 | 1.00 | 44.76 | 5-2b |
| ATOM | 2870 | C12 | 5-2b | 2 | 17.708 | 15.078 | 109.368 | 1.00 | 52.53 | 5-2b |
| ATOM | 2871 | O13 | 5-2b | 2 | 16.238 | 11.708 | 113.800 | 1.00 | 23.44 | 5-2b |
| ATOM | 2872 | C14 | 5-2b | 2 | 16.264 | 15.498 | 109.536 | 1.00 | 70.42 | 5-2b |
| ATOM | 2873 | O15 | 5-2b | 2 | 15.927 | 16.837 | 109.475 | 1.00 | 104.53 | 5-2b |
| ATOM | 2874 | C16 | 5-2b | 2 | 14.579 | 17.475 | 109.627 | 1.00 | 95.04 | 5-2b |
| ATOM | 2875 | C17 | 5-2b | 2 | 14.646 | 19.021 | 109.575 | 1.00 | 97.91 | 5-2b |
| ATOM | 2876 | C18 | 5-2b | 2 | 18.590 | 17.336 | 108.468 | 1.00 | 43.13 | 5-2b |
| ATOM | 2877 | O19 | 5-2b | 2 | 15.462 | 14.612 | 109.721 | 1.00 | 72.50 | 5-2b |
| ATOM | 2878 | S20 | 5-2b | 2 | 21.688 | 13.451 | 108.038 | 1.00 | 18.17 | 5-2b |
| ATOM | 2624 | O | HOH | 1 | 20.805 | 10.444 | 96.618 | 1.00 | 3.59 | S |
| ATOM | 2625 | O | HOH | 6 | 18.478 | 8.895 | 97.954 | 1.00 | 22.75 | S |
| ATOM | 2626 | O | HOH | 7 | 8.678 | 16.203 | 114.749 | 1.00 | 5.86 | S |
| ATOM | 2627 | O | HOH | 8 | 15.946 | −1.691 | 94.899 | 1.00 | 5.80 | S |
| ATOM | 2628 | O | HOH | 11 | 21.220 | 17.072 | 106.339 | 1.00 | 1.72 | S |
| ATOM | 2629 | O | HOH | 13 | 14.805 | 10.449 | 99.917 | 1.00 | 8.07 | S |
| ATOM | 2630 | O | HOH | 16 | 13.355 | −2.493 | 95.064 | 1.00 | 7.03 | S |
| ATOM | 2631 | O | HOH | 19 | 21.262 | 3.695 | 111.999 | 1.00 | 8.18 | S |
| ATOM | 2632 | O | HOH | 20 | 10.684 | 13.846 | 117.065 | 1.00 | 18.83 | S |
| ATOM | 2633 | O | HOH | 25 | 21.216 | 2.976 | 93.758 | 1.00 | 14.00 | S |
| ATOM | 2634 | O | HOH | 27 | 24.932 | 11.371 | 102.192 | 1.00 | 7.13 | S |
| ATOM | 2635 | O | HOH | 34 | 15.711 | 22.783 | 114.948 | 1.00 | 8.16 | S |
| ATOM | 2636 | O | HOH | 35 | 31.658 | 6.477 | 79.773 | 1.00 | 16.68 | S |
| ATOM | 2637 | O | HOH | 36 | 16.262 | 7.930 | 95.115 | 1.00 | 13.14 | S |
| ATOM | 2638 | O | HOH | 38 | 15.341 | −0.450 | 103.081 | 1.00 | 3.96 | S |
| ATOM | 2639 | O | HOH | 40 | 20.527 | 12.061 | 101.135 | 1.00 | 13.66 | S |
| ATOM | 2640 | O | HOH | 42 | 31.548 | 4.510 | 82.184 | 1.00 | 13.63 | S |
| ATOM | 2641 | O | HOH | 44 | 20.139 | 3.415 | 109.317 | 1.00 | 9.63 | S |
| ATOM | 2642 | O | HOH | 46 | 38.748 | 2.216 | 117.615 | 1.00 | 16.12 | S |
| ATOM | 2643 | O | HOH | 48 | 37.332 | 6.832 | 98.871 | 1.00 | 20.54 | S |
| ATOM | 2644 | O | HOH | 50 | 15.243 | 1.107 | 105.237 | 1.00 | 7.71 | S |
| ATOM | 2645 | O | HOH | 52 | 23.362 | 13.594 | 103.308 | 1.00 | 16.03 | S |
| ATOM | 2646 | O | HOH | 54 | 24.373 | 1.678 | 79.508 | 1.00 | 21.19 | S |
| ATOM | 2647 | O | HOH | 55 | 38.272 | 4.890 | 80.366 | 1.00 | 15.34 | S |
| ATOM | 2648 | O | HOH | 60 | 28.231 | 24.639 | 95.411 | 1.00 | 10.59 | S |
| ATOM | 2649 | O | HOH | 61 | 39.120 | 8.121 | 96.836 | 1.00 | 17.30 | S |
| ATOM | 2650 | O | HOH | 63 | 18.805 | 15.804 | 105.109 | 1.00 | 24.81 | S |
| ATOM | 2651 | O | HOH | 64 | 40.943 | 11.048 | 89.550 | 1.00 | 24.53 | S |
| ATOM | 2652 | O | HOH | 68 | 31.035 | 20.952 | 88.723 | 1.00 | 17.53 | S |
| ATOM | 2653 | O | HOH | 69 | 19.610 | −3.671 | 118.241 | 1.00 | 28.77 | S |
| ATOM | 2654 | O | HOH | 70 | 23.256 | 19.519 | 117.749 | 1.00 | 12.03 | S |
| ATOM | 2655 | O | HOH | 71 | 21.279 | 14.920 | 97.265 | 1.00 | 17.07 | S |
| ATOM | 2656 | O | HOH | 72 | 11.571 | 8.465 | 98.099 | 1.00 | 17.54 | S |
| ATOM | 2657 | O | HOH | 73 | 0.219 | −7.157 | 96.638 | 1.00 | 36.34 | S |
| ATOM | 2658 | O | HOH | 74 | 14.061 | −2.365 | 107.352 | 1.00 | 17.49 | S |
| ATOM | 2659 | O | HOH | 75 | 38.428 | 6.714 | 101.400 | 1.00 | 20.61 | S |
| ATOM | 2660 | O | HOH | 76 | 28.147 | 6.297 | 79.763 | 1.00 | 6.93 | S |
| ATOM | 2661 | O | HOH | 78 | 16.520 | −15.702 | 110.664 | 1.00 | 42.69 | S |
| ATOM | 2662 | O | HOH | 79 | 40.740 | 11.793 | 96.499 | 1.00 | 19.31 | S |
| ATOM | 2663 | O | HOH | 82 | 38.334 | −6.005 | 104.252 | 1.00 | 25.92 | S |
| ATOM | 2664 | O | HOH | 83 | 28.296 | 4.768 | 77.136 | 1.00 | 31.56 | S |
| ATOM | 2665 | O | HOH | 84 | 14.008 | 16.450 | 94.704 | 1.00 | 5.75 | S |
| ATOM | 2666 | O | HOH | 87 | 45.629 | 7.251 | 110.783 | 1.00 | 17.29 | S |
| ATOM | 2667 | O | HOH | 90 | 13.592 | 18.093 | 92.309 | 1.00 | 13.66 | S |
| ATOM | 2668 | O | HOH | 91 | 9.122 | 2.181 | 96.091 | 1.00 | 36.98 | S |
| ATOM | 2669 | O | HOH | 92 | 16.369 | 12.885 | 106.048 | 1.00 | 20.85 | S |
| ATOM | 2670 | O | HOH | 93 | 13.386 | 21.050 | 89.915 | 1.00 | 17.97 | S |
| ATOM | 2671 | O | HOH | 94 | 11.913 | 22.331 | 96.952 | 1.00 | 21.35 | S |
| ATOM | 2672 | O | HOH | 95 | 20.093 | −2.163 | 89.951 | 1.00 | 16.99 | S |
| ATOM | 2673 | O | HOH | 96 | 17.551 | −0.999 | 87.296 | 1.00 | 26.38 | S |
| ATOM | 2674 | O | HOH | 97 | 20.767 | 15.478 | 84.877 | 1.00 | 51.52 | S |
| ATOM | 2675 | O | HOH | 99 | 35.477 | 1.749 | 79.785 | 1.00 | 19.87 | S |
| ATOM | 2676 | O | HOH | 101 | 21.955 | 8.778 | 118.594 | 1.00 | 28.07 | S |
| ATOM | 2677 | O | HOH | 102 | 40.041 | 5.064 | 84.678 | 1.00 | 16.03 | S |
| ATOM | 2678 | O | HOH | 104 | 36.377 | −3.662 | 102.275 | 1.00 | 18.75 | S |
| ATOM | 2679 | O | HOH | 106 | 3.852 | 11.665 | 120.058 | 1.00 | 30.71 | S |
| ATOM | 2680 | O | HOH | 108 | 39.673 | −0.150 | 74.200 | 1.00 | 46.52 | S |
| ATOM | 2681 | O | HOH | 110 | 6.144 | −12.000 | 92.235 | 1.00 | 50.82 | S |
| ATOM | 2682 | O | HOH | 111 | 30.628 | 20.566 | 102.526 | 1.00 | 21.67 | S |
| ATOM | 2683 | O | HOH | 112 | 30.065 | 26.389 | 96.506 | 1.00 | 17.19 | S |
| ATOM | 2684 | O | HOH | 113 | 14.004 | 8.985 | 104.371 | 1.00 | 25.20 | S |
| ATOM | 2685 | O | HOH | 114 | 33.791 | 0.715 | 74.652 | 1.00 | 19.53 | S |
| ATOM | 2686 | O | HOH | 117 | 22.111 | 19.027 | 120.746 | 1.00 | 38.73 | S |
| ATOM | 2687 | O | HOH | 118 | 26.607 | 0.227 | 84.656 | 1.00 | 17.38 | S |
| ATOM | 2688 | O | HOH | 121 | 21.035 | −9.445 | 110.275 | 1.00 | 13.05 | S |
| ATOM | 2689 | O | HOH | 122 | 32.184 | 14.826 | 101.349 | 1.00 | 11.39 | S |
| ATOM | 2690 | O | HOH | 123 | 17.599 | −1.616 | 90.813 | 1.00 | 13.59 | S |
| ATOM | 2691 | O | HOH | 124 | 34.130 | 25.646 | 110.137 | 1.00 | 23.55 | S |
| ATOM | 2692 | O | HOH | 126 | 9.990 | −6.133 | 95.389 | 1.00 | 15.79 | S |
| ATOM | 2693 | O | HOH | 129 | 3.202 | −12.862 | 94.601 | 1.00 | 59.83 | S |
| ATOM | 2694 | O | HOH | 130 | 13.955 | 10.696 | 95.694 | 1.00 | 19.43 | S |
| ATOM | 2695 | O | HOH | 131 | 31.703 | 25.858 | 98.664 | 1.00 | 24.88 | S |
| ATOM | 2696 | O | HOH | 132 | 35.057 | 22.912 | 85.606 | 1.00 | 40.74 | S |
| ATOM | 2697 | O | HOH | 134 | 15.475 | −7.722 | 86.631 | 1.00 | 12.20 | S |
| ATOM | 2698 | O | HOH | 135 | 17.594 | 16.623 | 102.663 | 1.00 | 23.55 | S |
| ATOM | 2699 | O | HOH | 136 | 7.395 | −14.251 | 99.064 | 1.00 | 49.69 | S |
| ATOM | 2700 | O | HOH | 137 | 16.245 | 22.597 | 107.873 | 1.00 | 19.89 | S |
| ATOM | 2701 | O | HOH | 139 | 9.431 | −0.664 | 90.038 | 1.00 | 31.01 | S |
| ATOM | 2702 | O | HOH | 145 | 19.183 | 30.020 | 93.555 | 1.00 | 40.54 | S |
| ATOM | 2703 | O | HOH | 146 | 27.383 | 12.738 | 122.250 | 1.00 | 22.34 | S |
| ATOM | 2704 | O | HOH | 148 | 39.078 | −6.174 | 93.184 | 1.00 | 34.51 | S |
| ATOM | 2705 | O | HOH | 149 | 49.726 | 3.941 | 96.574 | 1.00 | 41.42 | S |
| ATOM | 2706 | O | HOH | 151 | 13.531 | 20.213 | 113.505 | 1.00 | 35.47 | S |
| ATOM | 2707 | O | HOH | 152 | 49.848 | 18.275 | 102.636 | 1.00 | 39.85 | S |
| ATOM | 2708 | O | HOH | 153 | 27.728 | −14.666 | 103.176 | 1.00 | 32.11 | S |
| ATOM | 2709 | O | HOH | 154 | 17.610 | 7.968 | 89.633 | 1.00 | 32.29 | S |
| ATOM | 2710 | O | HOH | 155 | 16.723 | 19.937 | 85.776 | 1.00 | 24.59 | S |
| ATOM | 2711 | O | HOH | 158 | 31.015 | −3.720 | 75.821 | 1.00 | 31.57 | S |
| ATOM | 2712 | O | HOH | 159 | 39.461 | 15.014 | 103.524 | 1.00 | 34.83 | S |
| ATOM | 2713 | O | HOH | 164 | 45.236 | 2.614 | 116.065 | 1.00 | 33.66 | S |
| ATOM | 2714 | O | HOH | 166 | 28.893 | 5.418 | 123.561 | 1.00 | 30.64 | S |
| ATOM | 2715 | O | HOH | 167 | 35.887 | 12.107 | 99.622 | 1.00 | 11.12 | S |
| ATOM | 2716 | O | HOH | 168 | 29.323 | −10.874 | 107.683 | 1.00 | 39.92 | S |
| ATOM | 2717 | O | HOH | 170 | 33.078 | 22.456 | 122.206 | 1.00 | 27.20 | S |
| ATOM | 2718 | O | HOH | 171 | 6.377 | −23.385 | 91.461 | 1.00 | 39.35 | S |
| ATOM | 2719 | O | HOH | 175 | 38.059 | 24.742 | 100.957 | 1.00 | 44.52 | S |
| ATOM | 2720 | O | HOH | 179 | 12.119 | −0.723 | 109.488 | 1.00 | 28.60 | S |
| ATOM | 2721 | O | HOH | 184 | 35.206 | −9.022 | 104.290 | 1.00 | 21.93 | S |
| ATOM | 2722 | O | HOH | 186 | 5.690 | −6.930 | 88.872 | 1.00 | 26.18 | S |
| ATOM | 2723 | O | HOH | 187 | 3.662 | −13.329 | 100.868 | 1.00 | 25.44 | S |
| ATOM | 2724 | O | HOH | 188 | 8.547 | −5.057 | 88.499 | 1.00 | 31.53 | S |
| ATOM | 2725 | O | HOH | 189 | 13.396 | 13.012 | 123.817 | 1.00 | 23.03 | S |
| ATOM | 2726 | O | HOH | 190 | 37.857 | 10.497 | 99.808 | 1.00 | 16.10 | S |
| ATOM | 2727 | O | HOH | 191 | 15.390 | 0.870 | 75.556 | 1.00 | 32.35 | S |
| ATOM | 2728 | O | HOH | 192 | 24.877 | 12.484 | 84.150 | 1.00 | 33.77 | S |
| ATOM | 2729 | O | HOH | 195 | 7.560 | 1.921 | 103.939 | 1.00 | 24.38 | S |
| ATOM | 2730 | O | HOH | 197 | 38.275 | 6.762 | 75.942 | 1.00 | 34.75 | S |
| ATOM | 2731 | O | HOH | 198 | 11.981 | 14.135 | 109.242 | 1.00 | 26.93 | S |
| ATOM | 2732 | O | HOH | 199 | 29.034 | −13.318 | 94.699 | 1.00 | 32.78 | S |
| ATOM | 2733 | O | HOH | 201 | 33.413 | −10.638 | 103.290 | 1.00 | 31.96 | S |
| ATOM | 2734 | O | HOH | 203 | 25.859 | 12.342 | 87.393 | 1.00 | 39.56 | S |
| ATOM | 2735 | O | HOH | 205 | 21.304 | 4.617 | 78.647 | 1.00 | 17.67 | S |
| ATOM | 2736 | O | HOH | 207 | 23.255 | 12.937 | 88.372 | 1.00 | 28.66 | S |
| ATOM | 2737 | O | HOH | 208 | 7.965 | 2.363 | 93.256 | 1.00 | 39.90 | S |
| ATOM | 2738 | O | HOH | 210 | 7.291 | −19.119 | 97.337 | 1.00 | 39.55 | S |
| ATOM | 2739 | O | HOH | 211 | 23.200 | 15.157 | 105.669 | 1.00 | 3.65 | S |
| ATOM | 2740 | O | HOH | 212 | 16.820 | 11.748 | 98.364 | 1.00 | 4.40 | S |
| ATOM | 2741 | O | HOH | 215 | 37.029 | 15.874 | 102.172 | 1.00 | 9.34 | S |
| ATOM | 2742 | O | HOH | 217 | 45.218 | 10.237 | 90.158 | 1.00 | 50.32 | S |
| ATOM | 2743 | O | HOH | 220 | 46.617 | 4.288 | 108.402 | 1.00 | 29.26 | S |
| ATOM | 2744 | O | HOH | 221 | 18.955 | 8.984 | 95.378 | 1.00 | 23.41 | S |
| ATOM | 2745 | O | HOH | 223 | 22.905 | 6.137 | 118.403 | 1.00 | 15.81 | S |
| ATOM | 2746 | O | HOH | 225 | 2.959 | −6.265 | 97.196 | 1.00 | 46.93 | S |
| ATOM | 2747 | O | HOH | 226 | 11.436 | 16.916 | 109.490 | 1.00 | 15.86 | S |
| ATOM | 2748 | O | HOH | 228 | 16.698 | 14.117 | 102.916 | 1.00 | 25.42 | S |
| ATOM | 2749 | O | HOH | 229 | 14.674 | 21.461 | 106.079 | 1.00 | 26.44 | S |
| ATOM | 2750 | O | HOH | 232 | 21.595 | −5.809 | 87.827 | 1.00 | 14.15 | S |
| ATOM | 2751 | O | HOH | 233 | 11.151 | 17.123 | 115.185 | 1.00 | 32.57 | S |
| ATOM | 2752 | O | HOH | 238 | 29.371 | −3.075 | 77.740 | 1.00 | 19.94 | S |
| ATOM | 2753 | O | HOH | 241 | 13.508 | 12.891 | 99.625 | 1.00 | 20.34 | S |
| ATOM | 2754 | O | HOH | 243 | 17.423 | 4.974 | 118.567 | 1.00 | 24.32 | S |
| ATOM | 2755 | O | HOH | 244 | 21.246 | 6.736 | 82.924 | 1.00 | 39.07 | S |
| ATOM | 2756 | O | HOH | 245 | 11.590 | 19.689 | 98.284 | 1.00 | 19.24 | S |
| ATOM | 2757 | O | HOH | 247 | 51.802 | 9.068 | 117.095 | 1.00 | 55.38 | S |
| ATOM | 2758 | O | HOH | 251 | 8.180 | 5.024 | 99.128 | 1.00 | 31.61 | S |
| ATOM | 2759 | O | HOH | 252 | 21.300 | 12.368 | 98.575 | 1.00 | 31.29 | S |
| ATOM | 2760 | O | HOH | 253 | 41.894 | 8.695 | 97.607 | 1.00 | 30.47 | S |
| ATOM | 2761 | O | HOH | 254 | 23.625 | 0.733 | 121.375 | 1.00 | 27.92 | S |
| ATOM | 2762 | O | HOH | 255 | 29.438 | 14.355 | 123.667 | 1.00 | 26.17 | S |
| ATOM | 2763 | O | HOH | 256 | 20.446 | 10.316 | 116.657 | 1.00 | 34.15 | S |
| ATOM | 2764 | O | HOH | 257 | 11.975 | 9.878 | 91.516 | 1.00 | 18.84 | S |
| ATOM | 2765 | O | HOH | 260 | 13.789 | 3.056 | 113.975 | 1.00 | 23.75 | S |
| ATOM | 2766 | O | HOH | 262 | 7.623 | 13.572 | 124.008 | 1.00 | 30.74 | S |
| ATOM | 2767 | O | HOH | 263 | 20.395 | 4.227 | 81.694 | 1.00 | 33.87 | S |
| ATOM | 2768 | O | HOH | 266 | 34.255 | −0.467 | 81.343 | 1.00 | 30.08 | S |
| ATOM | 2769 | O | HOH | 268 | 45.417 | 1.198 | 105.917 | 1.00 | 33.79 | S |
| ATOM | 2770 | O | HOH | 271 | 15.540 | −18.971 | 104.185 | 1.00 | 36.81 | S |
| ATOM | 2771 | O | HOH | 272 | 31.560 | 28.306 | 95.365 | 1.00 | 25.41 | S |
| ATOM | 2772 | O | HOH | 273 | 10.820 | 11.774 | 124.773 | 1.00 | 27.96 | S |
| ATOM | 2773 | O | HOH | 275 | 16.259 | 16.032 | 106.228 | 1.00 | 15.83 | S |
| ATOM | 2774 | O | HOH | 279 | 14.255 | 23.209 | 104.198 | 1.00 | 21.24 | S |
| ATOM | 2775 | O | HOH | 280 | 14.152 | 22.369 | 109.944 | 1.00 | 30.26 | S |
| ATOM | 2776 | O | HOH | 281 | 28.645 | −13.914 | 110.927 | 1.00 | 35.08 | S |
| ATOM | 2777 | O | HOH | 283 | 15.855 | 18.951 | 102.400 | 1.00 | 31.06 | S |
| ATOM | 2778 | O | HOH | 288 | 15.557 | 2.812 | 116.261 | 1.00 | 19.13 | S |
| ATOM | 2779 | O | HOH | 290 | 52.550 | 19.096 | 99.218 | 1.00 | 47.57 | S |
| ATOM | 2780 | O | HOH | 291 | 26.202 | 14.680 | 81.794 | 1.00 | 53.97 | S |
| ATOM | 2781 | O | HOH | 294 | 20.086 | 20.598 | 120.312 | 1.00 | 37.20 | S |
| ATOM | 2782 | O | HOH | 295 | 6.012 | 19.892 | 120.875 | 1.00 | 18.20 | S |
| ATOM | 2783 | O | HOH | 296 | 30.916 | 30.335 | 103.939 | 1.00 | 37.71 | S |
| ATOM | 2784 | O | HOH | 297 | 46.048 | 18.195 | 120.452 | 1.00 | 43.25 | S |
| ATOM | 2785 | O | HOH | 299 | 31.569 | −9.610 | 101.042 | 1.00 | 32.15 | S |
| ATOM | 2786 | O | HOH | 300 | 21.162 | −3.401 | 87.125 | 1.00 | 32.61 | S |
| ATOM | 2787 | O | HOH | 303 | 9.761 | 2.577 | 112.502 | 1.00 | 27.58 | S |
| ATOM | 2788 | O | HOH | 305 | 32.066 | 25.918 | 112.422 | 1.00 | 32.24 | S |
| ATOM | 2789 | O | HOH | 307 | 33.480 | −2.576 | 83.015 | 1.00 | 27.49 | S |
| ATOM | 2790 | O | HOH | 308 | 2.984 | 13.923 | 120.708 | 1.00 | 31.57 | S |
| ATOM | 2791 | O | HOH | 309 | 34.596 | −15.790 | 94.772 | 1.00 | 43.06 | S |
| ATOM | 2792 | O | HOH | 310 | 34.476 | −4.326 | 104.147 | 1.00 | 46.76 | S |
| ATOM | 2793 | O | HOH | 313 | 18.109 | −9.045 | 87.036 | 1.00 | 25.07 | S |
| ATOM | 2794 | O | HOH | 314 | 2.837 | 9.810 | 121.659 | 1.00 | 42.28 | S |
| ATOM | 2795 | O | HOH | 315 | 13.698 | 1.784 | 111.141 | 1.00 | 35.74 | S |
| ATOM | 2796 | O | HOH | 317 | 34.111 | 18.005 | 122.006 | 1.00 | 28.52 | S |
| ATOM | 2797 | O | HOH | 318 | 29.111 | −3.283 | 83.701 | 1.00 | 38.21 | S |
| ATOM | 2798 | O | HOH | 319 | 32.667 | 0.553 | 105.431 | 1.00 | 27.32 | S |
| ATOM | 2799 | O | HOH | 323 | 4.556 | −19.468 | 88.447 | 1.00 | 56.20 | S |
| ATOM | 2800 | O | HOH | 324 | −2.283 | −4.890 | 97.004 | 1.00 | 48.36 | S |
| ATOM | 2801 | O | HOH | 327 | 28.636 | −3.285 | 118.234 | 1.00 | 30.32 | S |
| ATOM | 2802 | O | HOH | 328 | 29.441 | 25.536 | 120.010 | 1.00 | 30.29 | S |
| ATOM | 2803 | O | HOH | 331 | 25.024 | 1.315 | 88.662 | 1.00 | 35.16 | S |
| ATOM | 2804 | O | HOH | 332 | 25.076 | 33.728 | 92.315 | 1.00 | 37.36 | S |
| ATOM | 2805 | O | HOH | 334 | 17.967 | 17.125 | 84.628 | 1.00 | 44.99 | S |
| ATOM | 2806 | O | HOH | 336 | 35.277 | −4.775 | 82.255 | 1.00 | 22.90 | S |
| ATOM | 2807 | O | HOH | 338 | 5.655 | −0.231 | 95.494 | 1.00 | 39.33 | S |
| ATOM | 2808 | O | HOH | 340 | 46.414 | −2.129 | 108.144 | 1.00 | 58.72 | S |
| ATOM | 2809 | O | HOH | 342 | 10.262 | −2.840 | 88.835 | 1.00 | 36.82 | S |
| ATOM | 2810 | O | HOH | 344 | 48.378 | −0.812 | 102.187 | 1.00 | 39.43 | S |
| ATOM | 2811 | O | HOH | 345 | 7.840 | 6.837 | 118.967 | 1.00 | 54.06 | S |
| ATOM | 2812 | O | HOH | 347 | 42.036 | −0.811 | 90.785 | 1.00 | 34.08 | S |
| ATOM | 2813 | O | HOH | 351 | 51.775 | 6.542 | 133.541 | 1.00 | 37.45 | S |
| ATOM | 2814 | O | HOH | 354 | 31.545 | 13.101 | 83.668 | 1.00 | 37.78 | S |
| ATOM | 2815 | O | HOH | 355 | 35.526 | 14.686 | 100.364 | 1.00 | 8.84 | S |
| ATOM | 2816 | O | HOH | 361 | 12.290 | 20.796 | 107.012 | 1.00 | 17.59 | S |
| ATOM | 2817 | O | HOH | 363 | 40.627 | 4.272 | 127.391 | 1.00 | 41.84 | S |
| ATOM | 2818 | O | HOH | 365 | 30.371 | −1.879 | 79.833 | 1.00 | 13.67 | S |
| ATOM | 2819 | O | HOH | 367 | 11.687 | 18.291 | 107.264 | 1.00 | 22.06 | S |
| ATOM | 2820 | O | HOH | 370 | 18.511 | 7.004 | 119.773 | 1.00 | 38.47 | S |
| ATOM | 2821 | O | HOH | 371 | 17.908 | 13.463 | 100.054 | 1.00 | 12.12 | S |
| ATOM | 2822 | O | HOH | 372 | 27.131 | −3.005 | 76.310 | 1.00 | 16.74 | S |
| ATOM | 2823 | O | HOH | 375 | 8.972 | 7.528 | 97.923 | 1.00 | 26.11 | S |
| ATOM | 2824 | O | HOH | 377 | 18.727 | 10.788 | 84.519 | 1.00 | 41.33 | S |
| ATOM | 2825 | O | HOH | 379 | 14.127 | 15.750 | 98.863 | 1.00 | 25.29 | S |
| ATOM | 2826 | O | HOH | 383 | 41.700 | 9.858 | 81.807 | 1.00 | 33.52 | S |
| ATOM | 2827 | O | HOH | 385 | 35.261 | 15.280 | 106.016 | 1.00 | 28.87 | S |
| ATOM | 2828 | O | HOH | 386 | 12.726 | 21.661 | 115.689 | 1.00 | 46.81 | S |
| ATOM | 2829 | O | HOH | 393 | 43.648 | 7.839 | 106.741 | 1.00 | 16.47 | S |
| ATOM | 2830 | O | HOH | 394 | 37.259 | 24.740 | 104.054 | 1.00 | 14.17 | S |
| ATOM | 2831 | O | HOH | 396 | 24.282 | −6.502 | 87.829 | 1.00 | 42.62 | S |
| ATOM | 2832 | O | HOH | 400 | 43.027 | −3.036 | 92.095 | 1.00 | 34.87 | S |
| ATOM | 2833 | O | HOH | 406 | 31.066 | −3.244 | 81.803 | 1.00 | 24.95 | S |
| ATOM | 2834 | O | HOH | 409 | 36.251 | 3.079 | 119.019 | 1.00 | 19.28 | S |
| ATOM | 2835 | O | HOH | 415 | 10.534 | 10.025 | 100.073 | 1.00 | 39.35 | S |
| ATOM | 2836 | O | HOH | 418 | 8.054 | 4.181 | 110.289 | 1.00 | 45.64 | S |
| ATOM | 2837 | O | HOH | 422 | 39.306 | 16.744 | 111.576 | 1.00 | 34.28 | S |
| ATOM | 2838 | O | HOH | 425 | 6.396 | 5.427 | 103.157 | 1.00 | 32.56 | S |
| ATOM | 2839 | O | HOH | 426 | 39.952 | 24.546 | 98.144 | 1.00 | 27.08 | S |
| ATOM | 2840 | O | HOH | 429 | 39.863 | 6.685 | 82.133 | 1.00 | 40.09 | S |
| ATOM | 2841 | O | HOH | 430 | 21.921 | 12.487 | 85.799 | 1.00 | 40.68 | S |
| ATOM | 2842 | O | HOH | 433 | 11.505 | 19.654 | 100.809 | 1.00 | 30.56 | S |
| ATOM | 2843 | O | HOH | 435 | 10.302 | 11.568 | 104.901 | 1.00 | 29.96 | S |
| ATOM | 2844 | O | HOH | 438 | 23.476 | −0.876 | 78.128 | 1.00 | 28.68 | S |
| ATOM | 2845 | O | HOH | 442 | 40.869 | 23.992 | 100.914 | 1.00 | 39.98 | S |
| ATOM | 2846 | O | HOH | 444 | 36.147 | 28.207 | 94.921 | 1.00 | 46.43 | S |
| ATOM | 2847 | O | HOH | 445 | 23.713 | 3.771 | 119.077 | 1.00 | 42.21 | S |
| ATOM | 2848 | O | HOH | 447 | 27.306 | −4.631 | 90.698 | 1.00 | 43.77 | S |
| ATOM | 2849 | O | HOH | 448 | 45.805 | 6.819 | 107.875 | 1.00 | 28.04 | S |
| ATOM | 2850 | O | HOH | 449 | 11.162 | 9.197 | 125.577 | 1.00 | 42.08 | S |
| ATOM | 2851 | O | HOH | 450 | 51.897 | 9.884 | 132.993 | 1.00 | 37.33 | S |
| ATOM | 2852 | O | HOH | 452 | 28.491 | 3.721 | 119.002 | 1.00 | 32.94 | S |
| ATOM | 2853 | O | HOH | 454 | 8.173 | 10.098 | 105.141 | 1.00 | 50.50 | S |
| ATOM | 2854 | O | HOH | 459 | 42.750 | 5.736 | 87.519 | 1.00 | 36.93 | S |
| ATOM | 2855 | O | HOH | 460 | 30.376 | 34.460 | 94.131 | 1.00 | 31.43 | S |
| ATOM | 2856 | O | HOH | 466 | 25.986 | 1.393 | 120.060 | 1.00 | 52.81 | S |
| ATOM | 2857 | O | HOH | 467 | 22.489 | −10.959 | 108.669 | 1.00 | 29.27 | S |
| ATOM | 2858 | O | HOH | 468 | 23.362 | −2.077 | 86.180 | 1.00 | 37.76 | S |
| END | | | | | | | | | | |

TABLE 2

| | |
|---|---|
| REMARK | 1 Compound 1-7_3dpb.pdb molecule B |
| !CRYST | 69.250 79.750 159.580 90.00 90.00 90.00 P212121 |

| | | | | | | | | | | |
|---|---|---|---|---|---|---|---|---|---|---|
| ATOM | 20 | CB | LYS | 17 | 24.352 | −12.458 | 60.280 | 1.00 | 51.00 | B |
| ATOM | 21 | CG | LYS | 17 | 22.874 | −12.492 | 59.882 | 1.00 | 53.34 | B |
| ATOM | 22 | CD | LYS | 17 | 22.663 | −12.316 | 58.375 | 1.00 | 53.77 | B |
| ATOM | 23 | CE | LYS | 17 | 23.197 | −13.512 | 57.582 | 1.00 | 54.85 | B |
| ATOM | 24 | NZ | LYS | 17 | 24.682 | −13.693 | 57.700 | 1.00 | 53.86 | B |
| ATOM | 25 | C | LYS | 17 | 24.606 | −10.105 | 59.443 | 1.00 | 47.83 | B |
| ATOM | 26 | O | LYS | 17 | 25.275 | −10.140 | 58.419 | 1.00 | 48.69 | B |
| ATOM | 27 | N | LYS | 17 | 24.345 | −10.549 | 61.888 | 1.00 | 49.93 | B |
| ATOM | 28 | CA | LYS | 17 | 24.911 | −11.048 | 60.601 | 1.00 | 49.15 | B |
| ATOM | 29 | N | ASN | 18 | 23.597 | −9.260 | 59.599 | 1.00 | 45.98 | B |
| ATOM | 30 | CA | ASN | 18 | 23.245 | −8.340 | 58.535 | 1.00 | 43.66 | B |
| ATOM | 31 | CB | ASN | 18 | 21.960 | −7.627 | 58.880 | 1.00 | 45.49 | B |
| ATOM | 32 | CG | ASN | 18 | 20.740 | −8.481 | 58.599 | 1.00 | 49.80 | B |
| ATOM | 33 | OD1 | ASN | 18 | 20.453 | −8.811 | 57.442 | 1.00 | 50.22 | B |
| ATOM | 34 | ND2 | ASN | 18 | 20.019 | −8.856 | 59.653 | 1.00 | 49.94 | B |
| ATOM | 35 | C | ASN | 18 | 24.338 | −7.336 | 58.180 | 1.00 | 41.30 | B |
| ATOM | 36 | O | ASN | 18 | 24.671 | −7.173 | 57.006 | 1.00 | 41.62 | B |
| ATOM | 37 | N | ILE | 19 | 24.906 | −6.669 | 59.179 | 1.00 | 37.77 | B |
| ATOM | 38 | CA | ILE | 19 | 25.949 | −5.679 | 58.928 | 1.00 | 34.25 | B |
| ATOM | 39 | CB | ILE | 19 | 26.325 | −4.966 | 60.253 | 1.00 | 35.25 | B |
| ATOM | 40 | CG2 | ILE | 19 | 26.548 | −5.988 | 61.346 | 1.00 | 38.29 | B |
| ATOM | 41 | CG1 | ILE | 19 | 27.581 | −4.139 | 60.078 | 1.00 | 35.22 | B |
| ATOM | 42 | CD1 | ILE | 19 | 28.042 | −3.487 | 61.347 | 1.00 | 36.16 | B |
| ATOM | 43 | C | ILE | 19 | 27.213 | −6.272 | 58.266 | 1.00 | 31.28 | B |
| ATOM | 44 | O | ILE | 19 | 27.730 | −7.287 | 58.722 | 1.00 | 31.52 | B |
| ATOM | 45 | N | GLN | 20 | 27.699 | −5.639 | 57.194 | 1.00 | 27.50 | B |
| ATOM | 46 | CA | GLN | 20 | 28.903 | −6.091 | 56.483 | 1.00 | 26.14 | B |
| ATOM | 47 | CB | GLN | 20 | 28.889 | −5.603 | 54.996 | 1.00 | 25.10 | B |
| ATOM | 48 | CG | GLN | 20 | 30.276 | −5.495 | 54.347 | 1.00 | 27.01 | B |
| ATOM | 49 | CD | GLN | 20 | 30.232 | −5.169 | 52.843 | 1.00 | 29.81 | B |
| ATOM | 50 | OE1 | GLN | 20 | 29.920 | −6.026 | 52.016 | 1.00 | 30.67 | B |
| ATOM | 51 | NE2 | GLN | 20 | 30.546 | −3.924 | 52.493 | 1.00 | 30.62 | B |
| ATOM | 52 | C | GLN | 20 | 30.162 | −5.567 | 57.176 | 1.00 | 25.43 | B |
| ATOM | 53 | O | GLN | 20 | 30.211 | −4.398 | 57.561 | 1.00 | 27.09 | B |
| ATOM | 54 | N | VAL | 21 | 31.176 | −6.426 | 57.327 | 1.00 | 22.08 | B |
| ATOM | 55 | CA | VAL | 21 | 32.427 | −6.048 | 57.989 | 1.00 | 18.37 | B |
| ATOM | 56 | CB | VAL | 21 | 32.472 | −6.584 | 59.471 | 1.00 | 19.87 | B |
| ATOM | 57 | CG1 | VAL | 21 | 33.802 | −6.230 | 60.125 | 1.00 | 16.85 | B |
| ATOM | 58 | CG2 | VAL | 21 | 31.300 | −6.004 | 60.291 | 1.00 | 14.97 | B |
| ATOM | 59 | C | VAL | 21 | 33.648 | −6.567 | 57.221 | 1.00 | 18.19 | B |
| ATOM | 60 | O | VAL | 21 | 33.848 | −7.771 | 57.081 | 1.00 | 16.60 | B |
| ATOM | 61 | N | VAL | 22 | 34.457 | −5.637 | 56.722 | 1.00 | 17.58 | B |
| ATOM | 62 | CA | VAL | 22 | 35.651 | −5.965 | 55.967 | 1.00 | 15.68 | B |
| ATOM | 63 | CB | VAL | 22 | 35.568 | −5.385 | 54.532 | 1.00 | 17.56 | B |
| ATOM | 64 | CG1 | VAL | 22 | 34.305 | −5.889 | 53.846 | 1.00 | 17.79 | B |
| ATOM | 65 | CG2 | VAL | 22 | 35.553 | −3.863 | 54.575 | 1.00 | 17.41 | B |
| ATOM | 66 | C | VAL | 22 | 36.869 | −5.396 | 56.693 | 1.00 | 16.43 | B |
| ATOM | 67 | O | VAL | 22 | 36.746 | −4.502 | 57.549 | 1.00 | 14.89 | B |
| ATOM | 68 | N | VAL | 23 | 38.038 | −5.936 | 56.358 | 1.00 | 14.83 | B |
| ATOM | 69 | CA | VAL | 23 | 39.304 | −5.534 | 56.972 | 1.00 | 13.82 | B |
| ATOM | 70 | CB | VAL | 23 | 39.935 | −6.745 | 57.768 | 1.00 | 13.54 | B |
| ATOM | 71 | CG1 | VAL | 23 | 41.330 | −6.405 | 58.282 | 1.00 | 6.83 | B |
| ATOM | 72 | CG2 | VAL | 23 | 39.034 | −7.112 | 58.944 | 1.00 | 13.12 | B |
| ATOM | 73 | C | VAL | 23 | 40.304 | −5.023 | 55.928 | 1.00 | 13.37 | B |
| ATOM | 74 | O | VAL | 23 | 40.414 | −5.576 | 54.835 | 1.00 | 10.49 | B |
| ATOM | 75 | N | ARG | 24 | 41.008 | −3.944 | 56.256 | 1.00 | 14.76 | B |
| ATOM | 76 | CA | ARG | 24 | 42.019 | −3.407 | 55.346 | 1.00 | 17.25 | B |
| ATOM | 77 | CB | ARG | 24 | 41.577 | −2.087 | 54.700 | 1.00 | 14.29 | B |
| ATOM | 78 | CG | ARG | 24 | 42.528 | −1.660 | 53.590 | 1.00 | 12.98 | B |
| ATOM | 79 | CD | ARG | 24 | 42.331 | −0.225 | 53.130 | 1.00 | 9.77 | B |
| ATOM | 80 | NE | ARG | 24 | 42.978 | −0.006 | 51.838 | 1.00 | 9.97 | B |
| ATOM | 81 | CZ | ARG | 24 | 42.881 | 1.111 | 51.112 | 1.00 | 9.72 | B |
| ATOM | 82 | NH1 | ARG | 24 | 42.165 | 2.143 | 51.544 | 1.00 | 3.96 | B |
| ATOM | 83 | NH2 | ARG | 24 | 43.477 | 1.177 | 49.923 | 1.00 | 8.75 | B |
| ATOM | 84 | C | ARG | 24 | 43.328 | −3.180 | 56.098 | 1.00 | 18.12 | B |
| ATOM | 85 | O | ARG | 24 | 43.384 | −2.408 | 57.055 | 1.00 | 16.79 | B |
| ATOM | 86 | N | CYS | 25 | 44.372 | −3.874 | 55.657 | 1.00 | 21.17 | B |
| ATOM | 87 | CA | CYS | 25 | 45.688 | −3.764 | 56.268 | 1.00 | 23.23 | B |
| ATOM | 88 | CB | CYS | 25 | 46.415 | −5.140 | 56.254 | 1.00 | 23.67 | B |
| ATOM | 89 | SG | CYS | 25 | 48.096 | −5.149 | 56.970 | 1.00 | 28.58 | B |
| ATOM | 90 | C | CYS | 25 | 46.464 | −2.764 | 55.443 | 1.00 | 24.61 | B |
| ATOM | 91 | O | CYS | 25 | 46.457 | −2.836 | 54.211 | 1.00 | 24.46 | B |
| ATOM | 92 | N | ARG | 26 | 47.116 | −1.818 | 56.109 | 1.00 | 25.36 | B |
| ATOM | 93 | CA | ARG | 26 | 47.897 | −0.829 | 55.380 | 1.00 | 27.69 | B |
| ATOM | 94 | CB | ARG | 26 | 48.087 | 0.458 | 56.219 | 1.00 | 26.88 | B |
| ATOM | 95 | CG | ARG | 26 | 49.165 | 0.361 | 57.300 | 1.00 | 25.37 | B |
| ATOM | 96 | CD | ARG | 26 | 49.817 | 1.722 | 57.544 | 1.00 | 26.81 | B |
| ATOM | 97 | NE | ARG | 26 | 51.181 | 1.599 | 58.060 | 1.00 | 30.34 | B |
| ATOM | 98 | CZ | ARG | 26 | 51.504 | 1.598 | 59.349 | 1.00 | 31.91 | B |
| ATOM | 99 | NH1 | ARG | 26 | 50.566 | 1.721 | 60.277 | 1.00 | 32.84 | B |
| ATOM | 100 | NH2 | ARG | 26 | 52.767 | 1.459 | 59.714 | 1.00 | 33.10 | B |
| ATOM | 101 | C | ARG | 26 | 49.268 | −1.423 | 55.072 | 1.00 | 29.73 | B |
| ATOM | 102 | O | ARG | 26 | 49.673 | −2.417 | 55.676 | 1.00 | 28.95 | B |
| ATOM | 103 | N | PRO | 27 | 49.991 | −0.832 | 54.108 | 1.00 | 31.27 | B |
| ATOM | 104 | CD | PRO | 27 | 49.498 | 0.108 | 53.083 | 1.00 | 32.66 | B |
| ATOM | 105 | CA | PRO | 27 | 51.327 | −1.324 | 53.757 | 1.00 | 32.62 | B |
| ATOM | 106 | CB | PRO | 27 | 51.452 | −0.937 | 52.287 | 1.00 | 31.65 | B |
| ATOM | 107 | CG | PRO | 27 | 50.745 | 0.369 | 52.235 | 1.00 | 31.82 | B |
| ATOM | 108 | C | PRO | 27 | 52.372 | −0.626 | 54.642 | 1.00 | 33.24 | B |
| ATOM | 109 | O | PRO | 27 | 52.065 | 0.364 | 55.311 | 1.00 | 33.16 | B |
| ATOM | 110 | N | PHE | 28 | 53.599 | −1.141 | 54.652 | 1.00 | 34.79 | B |
| ATOM | 111 | CA | PHE | 28 | 54.670 | −0.545 | 55.451 | 1.00 | 34.86 | B |
| ATOM | 112 | CB | PHE | 28 | 55.890 | −1.393 | 55.401 | 1.00 | 33.35 | B |
| ATOM | 113 | CG | PHE | 28 | 55.756 | −2.691 | 56.124 | 1.00 | 33.06 | B |
| ATOM | 114 | CD1 | PHE | 28 | 55.856 | −3.893 | 55.440 | 1.00 | 31.63 | B |
| ATOM | 115 | CD2 | PHE | 28 | 55.590 | −2.715 | 57.507 | 1.00 | 31.31 | B |
| ATOM | 116 | CE1 | PHE | 28 | 55.801 | −5.102 | 56.128 | 1.00 | 31.40 | B |
| ATOM | 117 | CE2 | PHE | 28 | 55.536 | −3.918 | 58.193 | 1.00 | 30.69 | B |
| ATOM | 118 | CZ | PHE | 28 | 55.644 | −5.112 | 57.500 | 1.00 | 29.86 | B |
| ATOM | 119 | C | PHE | 28 | 55.043 | 0.842 | 54.956 | 1.00 | 36.62 | B |
| ATOM | 120 | O | PHE | 28 | 55.102 | 1.080 | 53.752 | 1.00 | 36.72 | B |
| ATOM | 121 | N | ASN | 29 | 55.297 | 1.755 | 55.885 | 1.00 | 39.15 | B |
| ATOM | 122 | CA | ASN | 29 | 55.687 | 3.109 | 55.517 | 1.00 | 43.00 | B |
| ATOM | 123 | CB | ASN | 29 | 55.449 | 4.078 | 56.693 | 1.00 | 41.82 | B |
| ATOM | 124 | CG | ASN | 29 | 55.787 | 3.460 | 58.044 | 1.00 | 41.11 | B |
| ATOM | 125 | OD1 | ASN | 29 | 56.953 | 3.237 | 58.367 | 1.00 | 38.49 | B |
| ATOM | 126 | ND2 | ASN | 29 | 54.758 | 3.178 | 58.838 | 1.00 | 40.06 | B |
| ATOM | 127 | C | ASN | 29 | 57.160 | 3.083 | 55.130 | 1.00 | 46.95 | B |
| ATOM | 128 | O | ASN | 29 | 57.913 | 2.236 | 55.621 | 1.00 | 48.65 | B |
| ATOM | 129 | N | LEU | 30 | 57.554 | 3.998 | 54.243 | 1.00 | 49.22 | B |
| ATOM | 130 | CA | LEU | 30 | 58.930 | 4.106 | 53.751 | 1.00 | 49.70 | B |
| ATOM | 131 | CB | LEU | 30 | 59.142 | 5.490 | 53.121 | 1.00 | 49.24 | B |
| ATOM | 132 | CG | LEU | 30 | 60.429 | 5.757 | 52.341 | 1.00 | 49.29 | B |
| ATOM | 133 | CD1 | LEU | 30 | 60.294 | 7.104 | 51.640 | 1.00 | 49.07 | B |
| ATOM | 134 | CD2 | LEU | 30 | 61.643 | 5.740 | 53.264 | 1.00 | 49.24 | B |
| ATOM | 135 | C | LEU | 30 | 59.989 | 3.866 | 54.823 | 1.00 | 51.07 | B |
| ATOM | 136 | O | LEU | 30 | 60.877 | 3.032 | 54.649 | 1.00 | 50.68 | B |
| ATOM | 137 | N | ALA | 31 | 59.889 | 4.605 | 55.925 | 1.00 | 52.87 | B |
| ATOM | 138 | CA | ALA | 31 | 60.831 | 4.497 | 57.035 | 1.00 | 54.80 | B |
| ATOM | 139 | CB | ALA | 31 | 60.399 | 5.420 | 58.157 | 1.00 | 53.50 | B |
| ATOM | 140 | C | ALA | 31 | 61.011 | 3.077 | 57.576 | 1.00 | 56.55 | B |
| ATOM | 141 | O | ALA | 31 | 62.140 | 2.649 | 57.837 | 1.00 | 56.62 | B |
| ATOM | 142 | N | GLU | 32 | 59.906 | 2.354 | 57.751 | 1.00 | 59.00 | B |
| ATOM | 143 | CA | GLU | 32 | 59.958 | 0.989 | 58.272 | 1.00 | 61.92 | B |
| ATOM | 144 | CB | GLU | 32 | 58.625 | 0.631 | 58.999 | 1.00 | 61.49 | B |
| ATOM | 145 | CG | GLU | 32 | 57.413 | 0.441 | 58.094 | 1.00 | 60.80 | B |
| ATOM | 146 | CD | GLU | 32 | 56.101 | 0.376 | 58.872 | 1.00 | 59.87 | B |
| ATOM | 147 | OE1 | GLU | 32 | 55.038 | 0.196 | 58.242 | 1.00 | 58.45 | B |
| ATOM | 148 | OE2 | GLU | 32 | 56.129 | 0.514 | 60.115 | 1.00 | 60.23 | B |
| ATOM | 149 | C | GLU | 32 | 60.270 | −0.057 | 57.198 | 1.00 | 64.49 | B |
| ATOM | 150 | O | GLU | 32 | 60.610 | −1.199 | 57.522 | 1.00 | 64.33 | B |
| ATOM | 151 | N | ARG | 33 | 60.148 | 0.330 | 55.927 | 1.00 | 67.16 | B |
| ATOM | 152 | CA | ARG | 33 | 60.447 | −0.573 | 54.813 | 1.00 | 69.70 | B |
| ATOM | 153 | CB | ARG | 33 | 59.996 | 0.033 | 53.435 | 1.00 | 71.95 | B |
| ATOM | 154 | CG | ARG | 33 | 58.567 | 0.570 | 53.353 | 1.00 | 75.31 | B |
| ATOM | 155 | CD | ARG | 33 | 58.383 | 1.377 | 52.056 | 1.00 | 78.38 | B |
| ATOM | 156 | NE | ARG | 33 | 57.203 | 2.248 | 52.066 | 1.00 | 80.30 | B |
| ATOM | 157 | CZ | ARG | 33 | 56.937 | 3.167 | 51.136 | 1.00 | 80.67 | B |
| ATOM | 158 | NH1 | ARG | 33 | 57.766 | 3.345 | 50.114 | 1.00 | 79.70 | B |
| ATOM | 159 | NH2 | ARG | 33 | 55.841 | 3.913 | 51.226 | 1.00 | 80.30 | B |
| ATOM | 160 | C | ARG | 33 | 61.965 | −0.720 | 54.794 | 1.00 | 70.18 | B |
| ATOM | 161 | O | ARG | 33 | 62.502 | −1.813 | 54.599 | 1.00 | 70.13 | B |
| ATOM | 162 | N | LYS | 34 | 62.638 | 0.411 | 54.997 | 1.00 | 70.20 | B |
| ATOM | 163 | CA | LYS | 34 | 64.094 | 0.483 | 55.012 | 1.00 | 70.34 | B |
| ATOM | 164 | CB | LYS | 34 | 64.552 | 1.980 | 55.063 | 1.00 | 71.26 | B |
| ATOM | 165 | CG | LYS | 34 | 66.041 | 2.209 | 54.795 | 1.00 | 71.67 | B |
| ATOM | 166 | CD | LYS | 34 | 66.407 | 3.688 | 54.868 | 1.00 | 71.50 | B |
| ATOM | 167 | CE | LYS | 34 | 66.116 | 4.260 | 56.251 | 1.00 | 72.55 | B |
| ATOM | 168 | NZ | LYS | 34 | 66.513 | 5.694 | 56.388 | 1.00 | 72.95 | B |
| ATOM | 169 | C | LYS | 34 | 64.644 | −0.288 | 56.211 | 1.00 | 70.18 | B |
| ATOM | 170 | O | LYS | 34 | 65.707 | −0.915 | 56.123 | 1.00 | 70.68 | B |
| ATOM | 171 | N | ALA | 35 | 63.921 | −0.236 | 57.330 | 1.00 | 68.80 | B |
| ATOM | 172 | CA | ALA | 35 | 64.324 | −0.952 | 58.540 | 1.00 | 67.64 | B |
| ATOM | 173 | CB | ALA | 35 | 63.605 | −0.381 | 59.760 | 1.00 | 67.24 | B |
| ATOM | 174 | C | ALA | 35 | 63.958 | −2.424 | 58.356 | 1.00 | 66.54 | B |
| ATOM | 175 | O | ALA | 35 | 64.075 | −3.232 | 59.286 | 1.00 | 65.43 | B |
| ATOM | 176 | N | SER | 36 | 63.520 | −2.750 | 57.138 | 1.00 | 64.95 | B |
| ATOM | 177 | CA | SER | 36 | 63.113 | −4.099 | 56.770 | 1.00 | 63.77 | B |
| ATOM | 178 | CB | SER | 36 | 64.347 | −4.974 | 56.532 | 1.00 | 63.33 | B |
| ATOM | 179 | OG | SER | 36 | 65.136 | −4.438 | 55.481 | 1.00 | 61.84 | B |
| ATOM | 180 | C | SER | 36 | 62.240 | −4.670 | 57.879 | 1.00 | 63.32 | B |
| ATOM | 181 | O | SER | 36 | 62.731 | −5.313 | 58.810 | 1.00 | 63.79 | B |
| ATOM | 182 | N | ALA | 37 | 60.939 | −4.417 | 57.772 | 1.00 | 61.85 | B |
| ATOM | 183 | CA | ALA | 37 | 59.989 | −4.873 | 58.773 | 1.00 | 59.96 | B |
| ATOM | 184 | CB | ALA | 37 | 58.921 | −3.806 | 58.987 | 1.00 | 59.90 | B |
| ATOM | 185 | C | ALA | 37 | 59.344 | −6.219 | 58.442 | 1.00 | 58.87 | B |
| ATOM | 186 | O | ALA | 37 | 58.975 | −6.499 | 57.301 | 1.00 | 58.65 | B |
| ATOM | 187 | N | HIS | 38 | 59.215 | −7.038 | 59.479 | 1.00 | 57.20 | B |
| ATOM | 188 | CA | HIS | 38 | 58.638 | −8.378 | 59.411 | 1.00 | 54.48 | B |
| ATOM | 189 | CB | HIS | 38 | 59.315 | −9.263 | 60.513 | 1.00 | 56.18 | B |
| ATOM | 190 | CG | HIS | 38 | 59.436 | −8.582 | 61.851 | 1.00 | 56.74 | B |
| ATOM | 191 | CD2 | HIS | 38 | 59.058 | −8.977 | 63.092 | 1.00 | 57.32 | B |
| ATOM | 192 | ND1 | HIS | 38 | 60.024 | −7.344 | 62.011 | 1.00 | 55.67 | B |
| ATOM | 193 | CE1 | HIS | 38 | 60.005 | −7.006 | 63.288 | 1.00 | 56.12 | B |
| ATOM | 194 | NE2 | HIS | 38 | 59.424 | −7.980 | 63.967 | 1.00 | 57.53 | B |
| ATOM | 195 | C | HIS | 38 | 57.118 | −8.352 | 59.615 | 1.00 | 51.90 | B |
| ATOM | 196 | O | HIS | 38 | 56.642 | −8.343 | 60.754 | 1.00 | 52.05 | B |
| ATOM | 197 | N | SER | 39 | 56.356 | −8.350 | 58.523 | 1.00 | 47.82 | B |
| ATOM | 198 | CA | SER | 39 | 54.893 | −8.320 | 58.619 | 1.00 | 44.47 | B |
| ATOM | 199 | CB | SER | 39 | 54.255 | −8.336 | 57.219 | 1.00 | 43.58 | B |
| ATOM | 200 | OG | SER | 39 | 52.837 | −8.377 | 57.305 | 1.00 | 37.62 | B |
| ATOM | 201 | C | SER | 39 | 54.303 | −9.468 | 59.435 | 1.00 | 43.06 | B |
| ATOM | 202 | O | SER | 39 | 54.681 | −10.624 | 59.246 | 1.00 | 42.78 | B |
| ATOM | 203 | N | ILE | 40 | 53.373 | −9.144 | 60.334 | 1.00 | 41.07 | B |
| ATOM | 204 | CA | ILE | 40 | 52.727 | −10.162 | 61.157 | 1.00 | 39.33 | B |
| ATOM | 205 | CB | ILE | 40 | 52.660 | −9.761 | 62.665 | 1.00 | 39.17 | B |
| ATOM | 206 | CG2 | ILE | 40 | 54.063 | −9.542 | 63.215 | 1.00 | 38.53 | B |
| ATOM | 207 | CG1 | ILE | 40 | 51.824 | −8.511 | 62.858 | 1.00 | 39.67 | B |
| ATOM | 208 | CD1 | ILE | 40 | 51.496 | −8.238 | 64.319 | 1.00 | 38.82 | B |
| ATOM | 209 | C | ILE | 40 | 51.314 | −10.456 | 60.663 | 1.00 | 38.28 | B |
| ATOM | 210 | O | ILE | 40 | 50.591 | −11.249 | 61.265 | 1.00 | 37.83 | B |
| ATOM | 211 | N | VAL | 41 | 50.932 | −9.837 | 59.550 | 1.00 | 38.34 | B |
| ATOM | 212 | CA | VAL | 41 | 49.597 | −10.047 | 59.000 | 1.00 | 38.90 | B |
| ATOM | 213 | CB | VAL | 41 | 48.792 | −8.724 | 58.956 | 1.00 | 39.34 | B |
| ATOM | 214 | CG1 | VAL | 41 | 47.421 | −8.971 | 58.345 | 1.00 | 38.41 | B |
| ATOM | 215 | CG2 | VAL | 41 | 48.648 | −8.154 | 60.360 | 1.00 | 38.28 | B |
| ATOM | 216 | C | VAL | 41 | 49.535 | −10.683 | 57.612 | 1.00 | 38.55 | B |
| ATOM | 217 | O | VAL | 41 | 50.184 | −10.243 | 56.661 | 1.00 | 36.24 | B |
| ATOM | 218 | N | GLU | 42 | 48.728 | −11.729 | 57.513 | 1.00 | 40.08 | B |
| ATOM | 219 | CA | GLU | 42 | 48.528 | −12.433 | 56.255 | 1.00 | 42.70 | B |
| ATOM | 220 | CB | GLU | 42 | 48.931 | −13.916 | 56.393 | 1.00 | 45.52 | B |
| ATOM | 221 | CG | GLU | 42 | 50.403 | −14.215 | 56.163 | 1.00 | 47.68 | B |
| ATOM | 222 | CD | GLU | 42 | 50.783 | −15.636 | 56.578 | 1.00 | 50.75 | B |
| ATOM | 223 | OE1 | GLU | 42 | 49.991 | −16.576 | 56.323 | 1.00 | 52.01 | B |
| ATOM | 224 | OE2 | GLU | 42 | 51.883 | −15.816 | 57.151 | 1.00 | 51.85 | B |
| ATOM | 225 | C | GLU | 42 | 47.050 | −12.338 | 55.896 | 1.00 | 41.88 | B |
| ATOM | 226 | O | GLU | 42 | 46.193 | −12.740 | 56.683 | 1.00 | 42.51 | B |
| ATOM | 227 | N | CYS | 43 | 46.754 | −11.798 | 54.718 | 1.00 | 40.93 | B |
| ATOM | 228 | CA | CYS | 43 | 45.372 | −11.670 | 54.275 | 1.00 | 41.17 | B |
| ATOM | 229 | CB | CYS | 43 | 45.102 | −10.237 | 53.775 | 1.00 | 39.59 | B |
| ATOM | 230 | SG | CYS | 43 | 44.959 | −9.008 | 55.115 | 1.00 | 41.44 | B |
| ATOM | 231 | C | CYS | 43 | 45.033 | −12.682 | 53.185 | 1.00 | 42.27 | B |
| ATOM | 232 | O | CYS | 43 | 45.736 | −12.781 | 52.182 | 1.00 | 43.23 | B |
| ATOM | 233 | N | ASP | 44 | 43.953 | −13.435 | 53.394 | 1.00 | 43.10 | B |
| ATOM | 234 | CA | ASP | 44 | 43.504 | −14.444 | 52.436 | 1.00 | 43.06 | B |
| ATOM | 235 | CB | ASP | 44 | 43.392 | −15.831 | 53.138 | 1.00 | 45.99 | B |
| ATOM | 236 | CG | ASP | 44 | 43.414 | −16.999 | 52.151 | 1.00 | 46.99 | B |
| ATOM | 237 | OD1 | ASP | 44 | 42.678 | −16.948 | 51.139 | 1.00 | 48.57 | B |
| ATOM | 238 | OD2 | ASP | 44 | 44.167 | −17.971 | 52.398 | 1.00 | 44.91 | B |
| ATOM | 239 | C | ASP | 44 | 42.140 | −14.045 | 51.853 | 1.00 | 42.13 | B |
| ATOM | 240 | O | ASP | 44 | 41.093 | −14.446 | 52.363 | 1.00 | 39.99 | B |
| ATOM | 241 | N | PRO | 45 | 42.142 | −13.254 | 50.767 | 1.00 | 41.84 | B |
| ATOM | 242 | CD | PRO | 45 | 43.328 | −12.853 | 49.990 | 1.00 | 40.65 | B |
| ATOM | 243 | CA | PRO | 45 | 40.917 | −12.791 | 50.107 | 1.00 | 41.77 | B |
| ATOM | 244 | CB | PRO | 45 | 41.449 | −12.001 | 48.918 | 1.00 | 41.50 | B |
| ATOM | 245 | CG | PRO | 45 | 42.755 | −12.688 | 48.614 | 1.00 | 40.93 | B |
| ATOM | 246 | C | PRO | 45 | 39.940 | −13.893 | 49.690 | 1.00 | 42.90 | B |
| ATOM | 247 | O | PRO | 45 | 38.750 | −13.822 | 50.002 | 1.00 | 43.83 | B |
| ATOM | 248 | N | VAL | 46 | 40.429 | −14.908 | 48.985 | 1.00 | 42.74 | B |
| ATOM | 249 | CA | VAL | 46 | 39.554 | −15.990 | 48.552 | 1.00 | 42.50 | B |
| ATOM | 250 | CB | VAL | 46 | 40.348 | −17.109 | 47.854 | 1.00 | 41.92 | B |
| ATOM | 251 | CG1 | VAL | 46 | 39.428 | −18.269 | 47.531 | 1.00 | 40.40 | B |
| ATOM | 252 | CG2 | VAL | 46 | 40.983 | −16.574 | 46.581 | 1.00 | 41.19 | B |
| ATOM | 253 | C | VAL | 46 | 38.813 | −16.577 | 49.751 | 1.00 | 43.26 | B |
| ATOM | 254 | O | VAL | 46 | 37.587 | −16.736 | 49.730 | 1.00 | 43.10 | B |
| ATOM | 255 | N | ARG | 47 | 39.563 | −16.896 | 50.797 | 1.00 | 43.54 | B |
| ATOM | 256 | CA | ARG | 47 | 38.975 | −17.455 | 52.007 | 1.00 | 44.21 | B |
| ATOM | 257 | CB | ARG | 47 | 40.031 | −18.250 | 52.784 | 1.00 | 47.76 | B |
| ATOM | 258 | CG | ARG | 47 | 40.295 | −19.635 | 52.203 | 1.00 | 52.08 | B |
| ATOM | 259 | CD | ARG | 47 | 41.776 | −19.981 | 52.208 | 1.00 | 55.86 | B |
| ATOM | 260 | NE | ARG | 47 | 42.400 | −19.743 | 53.508 | 1.00 | 59.28 | B |
| ATOM | 261 | CZ | ARG | 47 | 42.043 | −20.346 | 54.638 | 1.00 | 60.15 | B |
| ATOM | 262 | NH1 | ARG | 47 | 41.056 | −21.237 | 54.639 | 1.00 | 60.50 | B |
| ATOM | 263 | NH2 | ARG | 47 | 42.674 | −20.051 | 55.770 | 1.00 | 60.66 | B |
| ATOM | 264 | C | ARG | 47 | 38.388 | −16.360 | 52.883 | 1.00 | 41.71 | B |
| ATOM | 265 | O | ARG | 47 | 37.673 | −16.643 | 53.845 | 1.00 | 40.72 | B |
| ATOM | 266 | N | LYS | 48 | 38.695 | −15.112 | 52.537 | 1.00 | 39.92 | B |
| ATOM | 267 | CA | LYS | 48 | 38.205 | −13.947 | 53.268 | 1.00 | 38.19 | B |
| ATOM | 268 | CB | LYS | 48 | 36.682 | −13.912 | 53.223 | 1.00 | 38.15 | B |
| ATOM | 269 | CG | LYS | 48 | 36.106 | −13.820 | 51.826 | 1.00 | 39.40 | B |
| ATOM | 270 | CD | LYS | 48 | 34.638 | −14.236 | 51.809 | 1.00 | 39.31 | B |
| ATOM | 271 | CE | LYS | 48 | 34.020 | −14.014 | 50.440 | 1.00 | 41.44 | B |
| ATOM | 272 | NZ | LYS | 48 | 34.853 | −14.620 | 49.354 | 1.00 | 42.78 | B |
| ATOM | 273 | C | LYS | 48 | 38.670 | −13.925 | 54.723 | 1.00 | 37.09 | B |
| ATOM | 274 | O | LYS | 48 | 37.905 | −13.563 | 55.617 | 1.00 | 37.31 | B |
| ATOM | 275 | N | GLU | 49 | 39.917 | −14.314 | 54.961 | 1.00 | 35.98 | B |
| ATOM | 276 | CA | GLU | 49 | 40.450 | −14.327 | 56.315 | 1.00 | 36.33 | B |
| ATOM | 277 | CB | GLU | 49 | 40.861 | −15.733 | 56.743 | 1.00 | 40.35 | B |
| ATOM | 278 | CG | GLU | 49 | 39.752 | −16.767 | 56.761 | 1.00 | 46.19 | B |
| ATOM | 279 | CD | GLU | 49 | 40.261 | −18.163 | 57.122 | 1.00 | 49.22 | B |
| ATOM | 280 | OE1 | GLU | 49 | 39.482 | −19.131 | 56.975 | 1.00 | 50.87 | B |
| ATOM | 281 | OE2 | GLU | 49 | 41.431 | −18.293 | 57.555 | 1.00 | 49.58 | B |
| ATOM | 282 | C | GLU | 49 | 41.669 | −13.444 | 56.445 | 1.00 | 35.96 | B |
| ATOM | 283 | O | GLU | 49 | 42.326 | −13.095 | 55.462 | 1.00 | 34.28 | B |
| ATOM | 284 | N | VAL | 50 | 41.967 | −13.097 | 57.685 | 1.00 | 34.47 | B |
| ATOM | 285 | CA | VAL | 50 | 43.122 | −12.292 | 57.999 | 1.00 | 34.53 | B |
| ATOM | 286 | CB | VAL | 50 | 42.704 | −10.858 | 58.439 | 1.00 | 32.83 | B |
| ATOM | 287 | CG1 | VAL | 50 | 41.653 | −10.918 | 59.512 | 1.00 | 30.31 | B |
| ATOM | 288 | CG2 | VAL | 50 | 43.916 | −10.092 | 58.929 | 1.00 | 32.98 | B |
| ATOM | 289 | C | VAL | 50 | 43.782 | −13.059 | 59.135 | 1.00 | 35.60 | B |
| ATOM | 290 | O | VAL | 50 | 43.136 | −13.367 | 60.130 | 1.00 | 36.44 | B |
| ATOM | 291 | N | SER | 51 | 45.054 | −13.411 | 58.976 | 1.00 | 36.72 | B |
| ATOM | 292 | CA | SER | 51 | 45.748 | −14.157 | 60.022 | 1.00 | 36.92 | B |
| ATOM | 293 | CB | SER | 51 | 46.320 | −15.481 | 59.447 | 1.00 | 37.59 | B |
| ATOM | 294 | OG | SER | 51 | 46.556 | −16.427 | 60.482 | 1.00 | 36.23 | B |
| ATOM | 295 | C | SER | 51 | 46.857 | −13.315 | 60.656 | 1.00 | 37.31 | B |
| ATOM | 296 | O | SER | 51 | 47.694 | −12.731 | 59.960 | 1.00 | 36.32 | B |
| ATOM | 297 | N | VAL | 52 | 46.852 | −13.265 | 61.984 | 1.00 | 37.74 | B |
| ATOM | 298 | CA | VAL | 52 | 47.817 | −12.474 | 62.735 | 1.00 | 39.56 | B |
| ATOM | 299 | CB | VAL | 52 | 47.092 | −11.558 | 63.749 | 1.00 | 38.44 | B |
| ATOM | 300 | CG1 | VAL | 52 | 48.090 | −10.668 | 64.454 | 1.00 | 37.83 | B |
| ATOM | 301 | CG2 | VAL | 52 | 46.041 | −10.737 | 63.042 | 1.00 | 37.78 | B |
| ATOM | 302 | C | VAL | 52 | 48.813 | −13.328 | 63.507 | 1.00 | 41.45 | B |
| ATOM | 303 | O | VAL | 52 | 48.429 | −14.296 | 64.167 | 1.00 | 41.94 | B |
| ATOM | 304 | N | ARG | 53 | 50.091 | −12.968 | 63.434 | 1.00 | 43.18 | B |
| ATOM | 305 | CA | ARG | 53 | 51.106 | −13.713 | 64.166 | 1.00 | 46.04 | B |
| ATOM | 306 | CB | ARG | 53 | 52.452 | −13.698 | 63.434 | 1.00 | 45.91 | B |
| ATOM | 307 | CG | ARG | 53 | 53.488 | −14.619 | 64.064 | 1.00 | 44.72 | B |
| ATOM | 308 | CD | ARG | 53 | 54.490 | −15.103 | 63.034 | 1.00 | 45.80 | B |
| ATOM | 309 | NE | ARG | 53 | 55.317 | −14.018 | 62.514 | 1.00 | 46.75 | B |
| ATOM | 310 | CZ | ARG | 53 | 56.036 | −14.095 | 61.398 | 1.00 | 45.30 | B |
| ATOM | 311 | NH1 | ARG | 53 | 56.028 | −15.209 | 60.675 | 1.00 | 44.24 | B |
| ATOM | 312 | NH2 | ARG | 53 | 56.765 | −13.056 | 61.011 | 1.00 | 44.19 | B |
| ATOM | 313 | C | ARG | 53 | 51.259 | −13.092 | 65.540 | 1.00 | 47.93 | B |
| ATOM | 314 | O | ARG | 53 | 51.466 | −11.884 | 65.667 | 1.00 | 48.40 | B |
| ATOM | 315 | N | THR | 54 | 51.156 | −13.929 | 66.565 | 1.00 | 49.62 | B |
| ATOM | 316 | CA | THR | 54 | 51.257 | −13.473 | 67.941 | 1.00 | 51.39 | B |
| ATOM | 317 | CB | THR | 54 | 49.941 | −13.744 | 68.683 | 1.00 | 51.01 | B |
| ATOM | 318 | OG1 | THR | 54 | 49.735 | −15.157 | 68.795 | 1.00 | 49.13 | B |
| ATOM | 319 | CG2 | THR | 54 | 48.775 | −13.144 | 67.914 | 1.00 | 51.53 | B |
| ATOM | 320 | C | THR | 54 | 52.391 | −14.139 | 68.709 | 1.00 | 52.60 | B |
| ATOM | 321 | O | THR | 54 | 52.439 | −14.058 | 69.933 | 1.00 | 53.07 | B |
| ATOM | 322 | N | GLY | 55 | 53.309 | −14.784 | 67.995 | 1.00 | 54.10 | B |
| ATOM | 323 | CA | GLY | 55 | 54.404 | −15.459 | 68.666 | 1.00 | 57.08 | B |
| ATOM | 324 | C | GLY | 55 | 55.721 | −15.519 | 67.914 | 1.00 | 59.62 | B |
| ATOM | 325 | O | GLY | 55 | 56.119 | −14.549 | 67.264 | 1.00 | 59.27 | B |
| ATOM | 326 | N | GLY | 56 | 56.393 | −16.668 | 68.016 | 1.00 | 60.97 | B |
| ATOM | 327 | CA | GLY | 56 | 57.682 | −16.880 | 67.372 | 1.00 | 62.99 | B |
| ATOM | 328 | C | GLY | 56 | 57.782 | −16.549 | 65.892 | 1.00 | 64.76 | B |
| ATOM | 329 | O | GLY | 56 | 56.940 | −15.828 | 65.350 | 1.00 | 66.18 | B |
| ATOM | 330 | N | LEU | 57 | 58.818 | −17.074 | 65.235 | 1.00 | 64.97 | B |
| ATOM | 331 | CA | LEU | 57 | 59.032 | −16.821 | 63.809 | 1.00 | 64.92 | B |
| ATOM | 332 | CB | LEU | 57 | 60.508 | −17.137 | 63.407 | 1.00 | 63.43 | B |
| ATOM | 333 | CG | LEU | 57 | 61.638 | −16.508 | 64.258 | 1.00 | 63.25 | B |
| ATOM | 334 | CD1 | LEU | 57 | 61.844 | −17.335 | 65.520 | 1.00 | 62.77 | B |
| ATOM | 335 | CD2 | LEU | 57 | 62.928 | −16.452 | 63.459 | 1.00 | 61.76 | B |
| ATOM | 336 | C | LEU | 57 | 58.080 | −17.654 | 62.951 | 1.00 | 65.79 | B |
| ATOM | 337 | O | LEU | 57 | 57.186 | −18.328 | 63.470 | 1.00 | 65.88 | B |
| ATOM | 338 | N | ALA | 58 | 58.269 | −17.597 | 61.636 | 1.00 | 65.65 | B |
| ATOM | 339 | CA | ALA | 58 | 57.435 | −18.356 | 60.712 | 1.00 | 65.12 | B |
| ATOM | 340 | CB | ALA | 58 | 57.687 | −17.891 | 59.286 | 1.00 | 65.82 | B |
| ATOM | 341 | C | ALA | 58 | 57.770 | −19.838 | 60.847 | 1.00 | 64.20 | B |
| ATOM | 342 | O | ALA | 58 | 56.953 | −20.709 | 60.525 | 1.00 | 64.59 | B |
| ATOM | 343 | N | ASP | 59 | 58.980 | −20.099 | 61.340 | 1.00 | 62.61 | B |
| ATOM | 344 | CA | ASP | 59 | 59.509 | −21.447 | 61.542 | 1.00 | 60.18 | B |
| ATOM | 345 | CB | ASP | 59 | 60.973 | −21.335 | 62.035 | 1.00 | 60.66 | B |
| ATOM | 346 | CG | ASP | 59 | 61.622 | −22.682 | 62.266 | 1.00 | 61.45 | B |
| ATOM | 347 | OD1 | ASP | 59 | 61.396 | −23.268 | 63.343 | 1.00 | 61.95 | B |
| ATOM | 348 | OD2 | ASP | 59 | 62.356 | −23.155 | 61.370 | 1.00 | 61.61 | B |
| ATOM | 349 | C | ASP | 59 | 58.663 | −22.274 | 62.519 | 1.00 | 58.06 | B |
| ATOM | 350 | O | ASP | 59 | 58.519 | −23.490 | 62.370 | 1.00 | 56.73 | B |
| ATOM | 351 | N | LYS | 60 | 58.109 | −21.591 | 63.513 | 1.00 | 55.07 | B |
| ATOM | 352 | CA | LYS | 60 | 57.258 | −22.200 | 64.528 | 1.00 | 52.63 | B |
| ATOM | 353 | CB | LYS | 60 | 58.107 | −23.079 | 65.525 | 1.00 | 51.66 | B |
| ATOM | 354 | CG | LYS | 60 | 57.301 | −23.696 | 66.672 | 1.00 | 51.86 | B |
| ATOM | 355 | CD | LYS | 60 | 58.046 | −24.839 | 67.368 | 1.00 | 51.88 | B |
| ATOM | 356 | CE | LYS | 60 | 59.349 | −24.373 | 68.011 | 1.00 | 53.18 | B |
| ATOM | 357 | NZ | LYS | 60 | 60.197 | −25.492 | 68.528 | 1.00 | 52.09 | B |
| ATOM | 358 | C | LYS | 60 | 56.615 | −21.023 | 65.248 | 1.00 | 51.19 | B |
| ATOM | 359 | O | LYS | 60 | 57.314 | −20.124 | 65.724 | 1.00 | 51.41 | B |
| ATOM | 360 | N | SER | 61 | 55.287 | −21.010 | 65.313 | 1.00 | 48.55 | B |
| ATOM | 361 | CA | SER | 61 | 54.599 | −19.905 | 65.960 | 1.00 | 45.99 | B |
| ATOM | 362 | CB | SER | 61 | 54.881 | −18.636 | 65.192 | 1.00 | 46.32 | B |
| ATOM | 363 | OG | SER | 61 | 54.559 | −18.803 | 63.820 | 1.00 | 44.99 | B |
| ATOM | 364 | C | SER | 61 | 53.092 | −20.082 | 66.086 | 1.00 | 45.35 | B |
| ATOM | 365 | O | SER | 61 | 52.491 | −20.950 | 65.449 | 1.00 | 44.81 | B |
| ATOM | 366 | N | SER | 62 | 52.488 | −19.242 | 66.922 | 1.00 | 43.72 | B |
| ATOM | 367 | CA | SER | 62 | 51.047 | −19.261 | 67.131 | 1.00 | 41.95 | B |
| ATOM | 368 | CB | SER | 62 | 50.738 | −19.050 | 68.592 | 1.00 | 41.39 | B |
| ATOM | 369 | OG | SER | 62 | 51.608 | −18.079 | 69.135 | 1.00 | 41.34 | B |
| ATOM | 370 | C | SER | 62 | 50.440 | −18.143 | 66.291 | 1.00 | 40.85 | B |
| ATOM | 371 | O | SER | 62 | 51.147 | −17.229 | 65.872 | 1.00 | 39.19 | B |
| ATOM | 372 | N | ARG | 63 | 49.138 | −18.221 | 66.031 | 1.00 | 40.24 | B |
| ATOM | 373 | CA | ARG | 63 | 48.461 | −17.207 | 65.226 | 1.00 | 38.90 | B |
| ATOM | 374 | CB | ARG | 63 | 48.630 | −17.514 | 63.695 | 1.00 | 39.76 | B |
| ATOM | 375 | CG | ARG | 63 | 50.074 | −17.554 | 63.205 | 1.00 | 41.62 | B |
| ATOM | 376 | CD | ARG | 63 | 50.149 | −17.897 | 61.725 | 1.00 | 43.20 | B |
| ATOM | 377 | NE | ARG | 63 | 49.763 | −16.776 | 60.866 | 1.00 | 46.47 | B |
| ATOM | 378 | CZ | ARG | 63 | 50.526 | −15.711 | 60.626 | 1.00 | 46.07 | B |
| ATOM | 379 | NH1 | ARG | 63 | 51.728 | −15.613 | 61.178 | 1.00 | 47.55 | B |
| ATOM | 380 | NH2 | ARG | 63 | 50.090 | −14.741 | 59.833 | 1.00 | 45.86 | B |
| ATOM | 381 | C | ARG | 63 | 46.976 | −17.131 | 65.558 | 1.00 | 37.75 | B |
| ATOM | 382 | O | ARG | 63 | 46.410 | −18.050 | 66.143 | 1.00 | 36.32 | B |
| ATOM | 383 | N | LYS | 64 | 46.356 | −16.019 | 65.174 | 1.00 | 37.15 | B |
| ATOM | 384 | CA | LYS | 64 | 44.931 | −15.788 | 65.400 | 1.00 | 35.14 | B |
| ATOM | 385 | CB | LYS | 64 | 44.737 | −14.607 | 66.342 | 1.00 | 36.48 | B |
| ATOM | 386 | CG | LYS | 64 | 45.236 | −14.826 | 67.760 | 1.00 | 37.70 | B |
| ATOM | 387 | CD | LYS | 64 | 44.174 | −15.510 | 68.604 | 1.00 | 40.04 | B |
| ATOM | 388 | CE | LYS | 64 | 44.488 | −15.408 | 70.087 | 1.00 | 40.04 | B |
| ATOM | 389 | NZ | LYS | 64 | 43.325 | −15.861 | 70.893 | 1.00 | 40.98 | B |
| ATOM | 390 | C | LYS | 64 | 44.316 | −15.467 | 64.041 | 1.00 | 33.82 | B |
| ATOM | 391 | O | LYS | 64 | 44.811 | −14.590 | 63.329 | 1.00 | 35.17 | B |
| ATOM | 392 | N | THR | 65 | 43.253 | −16.173 | 63.669 | 1.00 | 31.23 | B |
| ATOM | 393 | CA | THR | 65 | 42.619 | −15.928 | 62.377 | 1.00 | 30.10 | B |
| ATOM | 394 | CB | THR | 65 | 42.784 | −17.141 | 61.438 | 1.00 | 32.25 | B |
| ATOM | 395 | OG1 | THR | 65 | 44.171 | −17.498 | 61.357 | 1.00 | 32.66 | B |
| ATOM | 396 | CG2 | THR | 65 | 42.279 | −16.799 | 60.028 | 1.00 | 33.40 | B |
| ATOM | 397 | C | THR | 65 | 41.133 | −15.597 | 62.503 | 1.00 | 28.24 | B |
| ATOM | 398 | O | THR | 65 | 40.440 | −16.116 | 63.382 | 1.00 | 28.59 | B |
| ATOM | 399 | N | TYR | 66 | 40.648 | −14.720 | 61.630 | 1.00 | 24.28 | B |
| ATOM | 400 | CA | TYR | 66 | 39.244 | −14.335 | 61.665 | 1.00 | 22.45 | B |
| ATOM | 401 | CB | TYR | 66 | 39.045 | −12.976 | 62.362 | 1.00 | 19.03 | B |
| ATOM | 402 | CG | TYR | 66 | 39.783 | −12.804 | 63.674 | 1.00 | 16.05 | B |
| ATOM | 403 | CD1 | TYR | 66 | 41.158 | −12.594 | 63.697 | 1.00 | 11.74 | B |
| ATOM | 404 | CE1 | TYR | 66 | 41.829 | −12.377 | 64.894 | 1.00 | 13.31 | B |
| ATOM | 405 | CD2 | TYR | 66 | 39.094 | −12.802 | 64.891 | 1.00 | 15.60 | B |
| ATOM | 406 | CE2 | TYR | 66 | 39.753 | −12.586 | 66.097 | 1.00 | 13.06 | B |
| ATOM | 407 | CZ | TYR | 66 | 41.121 | −12.368 | 66.090 | 1.00 | 15.20 | B |
| ATOM | 408 | OH | TYR | 66 | 41.781 | −12.100 | 67.272 | 1.00 | 19.72 | B |
| ATOM | 409 | C | TYR | 66 | 38.666 | −14.241 | 60.271 | 1.00 | 22.39 | B |
| ATOM | 410 | O | TYR | 66 | 39.355 | −13.876 | 59.317 | 1.00 | 21.02 | B |
| ATOM | 411 | N | THR | 67 | 37.387 | −14.580 | 60.167 | 1.00 | 23.76 | B |
| ATOM | 412 | CA | THR | 67 | 36.678 | −14.523 | 58.900 | 1.00 | 25.75 | B |
| ATOM | 413 | CB | THR | 67 | 35.789 | −15.754 | 58.699 | 1.00 | 24.72 | B |
| ATOM | 414 | OG1 | THR | 67 | 36.607 | −16.923 | 58.702 | 1.00 | 28.23 | B |
| ATOM | 415 | CG2 | THR | 67 | 35.043 | −15.664 | 57.376 | 1.00 | 24.97 | B |
| ATOM | 416 | C | THR | 67 | 35.787 | −13.291 | 58.864 | 1.00 | 26.39 | B |
| ATOM | 417 | O | THR | 67 | 35.036 | −13.026 | 59.811 | 1.00 | 26.22 | B |
| ATOM | 418 | N | PHE | 68 | 35.899 | −12.538 | 57.775 | 1.00 | 26.28 | B |
| ATOM | 419 | CA | PHE | 68 | 35.091 | −11.342 | 57.565 | 1.00 | 27.23 | B |
| ATOM | 420 | CB | PHE | 68 | 35.942 | −10.056 | 57.673 | 1.00 | 25.89 | B |
| ATOM | 421 | CG | PHE | 68 | 36.634 | −9.893 | 58.997 | 1.00 | 27.52 | B |
| ATOM | 422 | CD1 | PHE | 68 | 37.873 | −10.485 | 59.230 | 1.00 | 26.70 | B |
| ATOM | 423 | CD2 | PHE | 68 | 36.037 | −9.161 | 60.023 | 1.00 | 26.12 | B |
| ATOM | 424 | CE1 | PHE | 68 | 38.501 | −10.350 | 60.464 | 1.00 | 25.62 | B |
| ATOM | 425 | CE2 | PHE | 68 | 36.662 | −9.025 | 61.258 | 1.00 | 25.03 | B |
| ATOM | 426 | CZ | PHE | 68 | 37.894 | −9.619 | 61.478 | 1.00 | 25.92 | B |
| ATOM | 427 | C | PHE | 68 | 34.492 | −11.434 | 56.171 | 1.00 | 27.19 | B |
| ATOM | 428 | O | PHE | 68 | 34.955 | −12.206 | 55.328 | 1.00 | 27.43 | B |
| ATOM | 429 | N | ASP | 69 | 33.470 | −10.631 | 55.926 | 1.00 | 26.71 | B |
| ATOM | 430 | CA | ASP | 69 | 32.805 | −10.629 | 54.636 | 1.00 | 27.55 | B |
| ATOM | 431 | CB | ASP | 69 | 31.660 | −9.635 | 54.684 | 1.00 | 27.61 | B |
| ATOM | 432 | CG | ASP | 69 | 30.623 | −10.019 | 55.735 | 1.00 | 28.58 | B |
| ATOM | 433 | OD1 | ASP | 69 | 30.578 | −9.403 | 56.831 | 1.00 | 27.66 | B |
| ATOM | 434 | OD2 | ASP | 69 | 29.865 | −10.972 | 55.461 | 1.00 | 28.48 | B |
| ATOM | 435 | C | ASP | 69 | 33.738 | −10.366 | 53.458 | 1.00 | 27.41 | B |
| ATOM | 436 | O | ASP | 69 | 33.455 | −10.771 | 52.334 | 1.00 | 27.23 | B |
| ATOM | 437 | N | MET | 70 | 34.861 | −9.710 | 53.732 | 1.00 | 28.30 | B |
| ATOM | 438 | CA | MET | 70 | 35.865 | −9.396 | 52.717 | 1.00 | 28.88 | B |
| ATOM | 439 | CB | MET | 70 | 35.424 | −8.213 | 51.821 | 1.00 | 30.69 | B |
| ATOM | 440 | CG | MET | 70 | 34.283 | −8.469 | 50.867 | 1.00 | 31.73 | B |
| ATOM | 441 | SD | MET | 70 | 33.894 | −6.957 | 49.923 | 1.00 | 36.68 | B |
| ATOM | 442 | CE | MET | 70 | 32.083 | −7.049 | 49.877 | 1.00 | 34.73 | B |
| ATOM | 443 | C | MET | 70 | 37.141 | −8.983 | 53.433 | 1.00 | 28.83 | B |
| ATOM | 444 | O | MET | 70 | 37.098 | −8.480 | 54.553 | 1.00 | 29.82 | B |
| ATOM | 445 | N | VAL | 71 | 38.274 | −9.188 | 52.780 | 1.00 | 27.33 | B |
| ATOM | 446 | CA | VAL | 71 | 39.553 | −8.812 | 53.349 | 1.00 | 26.23 | B |
| ATOM | 447 | CB | VAL | 71 | 40.291 | −10.021 | 54.003 | 1.00 | 27.99 | B |
| ATOM | 448 | CG1 | VAL | 71 | 39.635 | −10.381 | 55.319 | 1.00 | 28.32 | B |
| ATOM | 449 | CG2 | VAL | 71 | 40.264 | −11.219 | 53.076 | 1.00 | 28.60 | B |
| ATOM | 450 | C | VAL | 71 | 40.398 | −8.233 | 52.231 | 1.00 | 25.01 | B |
| ATOM | 451 | O | VAL | 71 | 40.363 | −8.713 | 51.100 | 1.00 | 24.55 | B |
| ATOM | 452 | N | PHE | 72 | 41.146 | −7.191 | 52.571 | 1.00 | 24.93 | B |
| ATOM | 453 | CA | PHE | 72 | 42.005 | −6.475 | 51.645 | 1.00 | 24.43 | B |
| ATOM | 454 | CB | PHE | 72 | 41.444 | −5.076 | 51.392 | 1.00 | 23.95 | B |
| ATOM | 455 | CG | PHE | 72 | 40.024 | −5.059 | 50.903 | 1.00 | 23.17 | B |
| ATOM | 456 | CD1 | PHE | 72 | 39.722 | −5.376 | 49.583 | 1.00 | 22.75 | B |
| ATOM | 457 | CD2 | PHE | 72 | 38.991 | −4.680 | 51.754 | 1.00 | 23.31 | B |
| ATOM | 458 | CE1 | PHE | 72 | 38.414 | −5.310 | 49.113 | 1.00 | 23.87 | B |
| ATOM | 459 | CE2 | PHE | 72 | 37.679 | −4.612 | 51.294 | 1.00 | 23.71 | B |
| ATOM | 460 | CZ | PHE | 72 | 37.389 | −4.927 | 49.970 | 1.00 | 24.15 | B |
| ATOM | 461 | C | PHE | 72 | 43.381 | −6.321 | 52.266 | 1.00 | 25.11 | B |
| ATOM | 462 | O | PHE | 72 | 43.522 | −5.683 | 53.312 | 1.00 | 26.80 | B |
| ATOM | 463 | N | GLY | 73 | 44.394 | −6.885 | 51.621 | 1.00 | 24.77 | B |
| ATOM | 464 | CA | GLY | 73 | 45.741 | −6.774 | 52.142 | 1.00 | 23.03 | B |
| ATOM | 465 | C | GLY | 73 | 46.352 | −5.450 | 51.143 | 1.00 | 26.33 | B |
| ATOM | 466 | O | GLY | 73 | 45.698 | −4.594 | 51.141 | 1.00 | 26.76 | B |
| ATOM | 467 | N | ALA | 74 | 47.626 | −5.284 | 52.062 | 1.00 | 27.88 | B |
| ATOM | 468 | CA | ALA | 74 | 46.335 | −4.054 | 51.752 | 1.00 | 28.98 | B |
| ATOM | 469 | CB | ALA | 74 | 49.690 | −4.074 | 52.427 | 1.00 | 29.52 | B |
| ATOM | 470 | C | ALA | 74 | 48.505 | −3.802 | 50.260 | 1.00 | 29.91 | B |
| ATOM | 471 | O | ALA | 74 | 49.037 | −2.773 | 49.865 | 1.00 | 31.84 | B |
| ATOM | 472 | N | SER | 75 | 48.051 | −4.726 | 49.426 | 1.00 | 31.43 | B |
| ATOM | 473 | CA | SER | 75 | 48.209 | −4.558 | 47.982 | 1.00 | 34.31 | B |
| ATOM | 474 | CB | SER | 75 | 48.382 | −5.914 | 47.318 | 1.00 | 32.52 | B |
| ATOM | 475 | OG | SER | 75 | 49.088 | −6.785 | 48.183 | 1.00 | 36.15 | B |
| ATOM | 476 | C | SER | 75 | 46.994 | −3.858 | 47.395 | 1.00 | 34.29 | B |
| ATOM | 477 | O | SER | 75 | 47.066 | −3.236 | 46.327 | 1.00 | 34.53 | B |
| ATOM | 478 | N | THR | 76 | 45.882 | −3.963 | 48.111 | 1.00 | 32.69 | B |
| ATOM | 479 | CA | THR | 76 | 44.635 | −3.364 | 47.675 | 1.00 | 32.77 | B |
| ATOM | 480 | CB | THR | 76 | 43.530 | −3.549 | 48.744 | 1.00 | 32.84 | B |
| ATOM | 481 | OG1 | THR | 76 | 43.612 | −4.863 | 49.305 | 1.00 | 31.95 | B |
| ATOM | 482 | CG2 | THR | 76 | 42.158 | −3.380 | 48.120 | 1.00 | 33.21 | B |
| ATOM | 483 | C | THR | 76 | 44.803 | −1.870 | 47.403 | 1.00 | 31.46 | B |
| ATOM | 484 | O | THR | 76 | 45.305 | −1.134 | 48.251 | 1.00 | 32.33 | B |
| ATOM | 485 | N | LYS | 77 | 44.394 | −1.430 | 46.218 | 1.00 | 29.15 | B |
| ATOM | 486 | CA | LYS | 77 | 44.469 | −0.015 | 45.875 | 1.00 | 27.33 | B |
| ATOM | 487 | CB | LYS | 77 | 44.906 | 0.155 | 44.423 | 1.00 | 29.39 | B |
| ATOM | 488 | CG | LYS | 77 | 46.342 | −0.341 | 44.187 | 1.00 | 32.84 | B |
| ATOM | 489 | CD | LYS | 77 | 46.949 | 0.180 | 42.884 | 1.00 | 36.59 | B |
| ATOM | 490 | CE | LYS | 77 | 46.241 | −0.349 | 41.627 | 1.00 | 38.03 | B |
| ATOM | 491 | NZ | LYS | 77 | 44.818 | 0.106 | 41.501 | 1.00 | 38.31 | B |
| ATOM | 492 | C | LYS | 77 | 43.096 | 0.625 | 46.134 | 1.00 | 25.52 | B |
| ATOM | 493 | O | LYS | 77 | 42.127 | −0.088 | 46.371 | 1.00 | 23.25 | B |
| ATOM | 494 | N | GLN | 78 | 43.018 | 1.956 | 46.115 | 1.00 | 24.22 | B |
| ATOM | 495 | CA | GLN | 78 | 41.759 | 2.652 | 46.398 | 1.00 | 22.43 | B |
| ATOM | 496 | CB | GLN | 78 | 41.935 | 4.177 | 46.226 | 1.00 | 22.53 | B |
| ATOM | 497 | CG | GLN | 78 | 43.014 | 4.799 | 47.088 | 1.00 | 21.23 | B |
| ATOM | 498 | CD | GLN | 78 | 42.603 | 4.953 | 48.539 | 1.00 | 20.15 | B |
| ATOM | 499 | OE1 | GLN | 78 | 42.235 | 3.988 | 49.192 | 1.00 | 18.03 | B |
| ATOM | 500 | NE2 | GLN | 78 | 42.661 | 6.178 | 49.045 | 1.00 | 21.65 | B |
| ATOM | 501 | C | GLN | 78 | 40.624 | 2.177 | 45.504 | 1.00 | 22.10 | B |
| ATOM | 502 | O | GLN | 78 | 39.533 | 1.839 | 45.986 | 1.00 | 20.46 | B |
| ATOM | 503 | N | ILE | 79 | 40.898 | 2.153 | 44.203 | 1.00 | 21.56 | B |
| ATOM | 504 | CA | ILE | 79 | 39.929 | 1.746 | 43.194 | 1.00 | 23.67 | B |
| ATOM | 505 | CB | ILE | 79 | 40.590 | 1.749 | 41.774 | 1.00 | 23.18 | B |
| ATOM | 506 | CG2 | ILE | 79 | 41.716 | 0.732 | 41.715 | 1.00 | 24.28 | B |
| ATOM | 507 | CG1 | ILE | 79 | 39.574 | 1.416 | 40.705 | 1.00 | 21.98 | B |
| ATOM | 508 | CD1 | ILE | 79 | 38.563 | 2.492 | 40.470 | 1.00 | 23.15 | B |
| ATOM | 509 | C | ILE | 79 | 39.303 | 0.366 | 43.475 | 1.00 | 25.91 | B |
| ATOM | 510 | O | ILE | 79 | 38.142 | 0.120 | 43.122 | 1.00 | 26.57 | B |
| ATOM | 511 | N | ASP | 80 | 40.061 | −0.527 | 44.107 | 1.00 | 24.45 | B |
| ATOM | 512 | CA | ASP | 80 | 39.547 | −1.857 | 44.416 | 1.00 | 25.05 | B |
| ATOM | 513 | CB | ASP | 80 | 40.694 | −2.832 | 44.721 | 1.00 | 25.59 | B |
| ATOM | 514 | CG | ASP | 80 | 41.691 | −2.928 | 43.588 | 1.00 | 26.46 | B |
| ATOM | 515 | OD1 | ASP | 80 | 41.248 | −2.925 | 42.414 | 1.00 | 26.20 | B |
| ATOM | 516 | OD2 | ASP | 80 | 42.912 | −3.016 | 43.877 | 1.00 | 27.35 | B |
| ATOM | 517 | C | ASP | 80 | 38.612 | −1.809 | 45.611 | 1.00 | 24.84 | B |
| ATOM | 518 | O | ASP | 80 | 37.638 | −2.553 | 45.686 | 1.00 | 23.83 | B |
| ATOM | 519 | N | VAL | 81 | 38.924 | −0.934 | 46.556 | 1.00 | 25.12 | B |
| ATOM | 520 | CA | VAL | 81 | 38.102 | −0.794 | 47.742 | 1.00 | 25.00 | B |
| ATOM | 521 | CB | VAL | 81 | 38.749 | 0.174 | 48.750 | 1.00 | 22.43 | B |
| ATOM | 522 | CG1 | VAL | 81 | 37.698 | 0.713 | 49.716 | 1.00 | 21.58 | B |
| ATOM | 523 | CG2 | VAL | 81 | 39.855 | −0.555 | 49.509 | 1.00 | 20.63 | B |
| ATOM | 524 | C | VAL | 81 | 36.753 | −0.250 | 47.320 | 1.00 | 27.16 | B |
| ATOM | 525 | O | VAL | 81 | 35.707 | −0.746 | 47.747 | 1.00 | 27.22 | B |
| ATOM | 526 | N | TYR | 82 | 36.792 | 0.769 | 46.464 | 1.00 | 27.98 | B |
| ATOM | 527 | CA | TYR | 82 | 35.580 | 1.406 | 45.987 | 1.00 | 28.04 | B |
| ATOM | 528 | CB | TYR | 82 | 35.922 | 2.661 | 45.125 | 1.00 | 27.34 | B |
| ATOM | 529 | CG | TYR | 82 | 34.681 | 3.366 | 44.637 | 1.00 | 26.71 | B |
| ATOM | 530 | CD1 | TYR | 82 | 34.262 | 3.252 | 43.315 | 1.00 | 26.63 | B |
| ATOM | 531 | CE1 | TYR | 82 | 33.054 | 3.808 | 42.893 | 1.00 | 29.11 | B |
| ATOM | 532 | CD2 | TYR | 82 | 33.866 | 4.063 | 45.529 | 1.00 | 27.27 | B |
| ATOM | 533 | CE2 | TYR | 82 | 32.660 | 4.620 | 45.128 | 1.00 | 28.67 | B |
| ATOM | 534 | CZ | TYR | 82 | 32.257 | 4.488 | 43.809 | 1.00 | 30.95 | B |
| ATOM | 535 | OH | TYR | 82 | 31.047 | 5.021 | 43.418 | 1.00 | 34.58 | B |
| ATOM | 536 | C | TYR | 82 | 34.705 | 0.454 | 45.183 | 1.00 | 29.38 | B |
| ATOM | 537 | O | TYR | 82 | 33.498 | 0.322 | 45.448 | 1.00 | 28.44 | B |
| ATOM | 538 | N | ARG | 83 | 35.312 | −0.212 | 44.206 | 1.00 | 30.12 | B |
| ATOM | 539 | CA | ARG | 83 | 34.569 | −1.136 | 43.365 | 1.00 | 32.33 | B |
| ATOM | 540 | CB | ARG | 83 | 35.475 | −1.667 | 42.238 | 1.00 | 32.84 | B |
| ATOM | 541 | CG | ARG | 83 | 35.814 | −0.610 | 41.177 | 1.00 | 36.78 | B |
| ATOM | 542 | CD | ARG | 83 | 36.995 | −1.024 | 40.298 | 1.00 | 39.59 | B |
| ATOM | 543 | NE | ARG | 83 | 36.692 | −2.180 | 39.459 | 1.00 | 45.16 | B |
| ATOM | 544 | CZ | ARG | 83 | 36.158 | −2.110 | 38.242 | 1.00 | 46.77 | B |
| ATOM | 545 | NH1 | ARG | 83 | 35.870 | −0.930 | 37.706 | 1.00 | 47.42 | B |
| ATOM | 546 | NH2 | ARG | 83 | 35.897 | −3.226 | 37.567 | 1.00 | 47.17 | B |
| ATOM | 547 | C | ARG | 83 | 33.930 | −2.291 | 44.142 | 1.00 | 32.86 | B |
| ATOM | 548 | O | ARG | 83 | 32.786 | −2.658 | 43.866 | 1.00 | 34.02 | B |
| ATOM | 549 | N | SER | 84 | 34.648 | −2.834 | 45.125 | 1.00 | 32.13 | B |
| ATOM | 550 | CA | SER | 84 | 34.159 | −3.959 | 45.933 | 1.00 | 30.95 | B |
| ATOM | 551 | CB | SER | 84 | 35.347 | −4.712 | 46.558 | 1.00 | 32.34 | B |
| ATOM | 552 | OG | SER | 84 | 36.301 | −5.060 | 45.568 | 1.00 | 37.12 | B |
| ATOM | 553 | C | SER | 84 | 33.186 | −3.593 | 47.046 | 1.00 | 29.09 | B |
| ATOM | 554 | O | SER | 84 | 32.151 | −4.241 | 47.225 | 1.00 | 29.03 | B |
| ATOM | 555 | N | VAL | 85 | 33.522 | −2.570 | 47.815 | 1.00 | 27.74 | B |
| ATOM | 556 | CA | VAL | 85 | 32.652 | −2.176 | 48.911 | 1.00 | 27.01 | B |
| ATOM | 557 | CB | VAL | 85 | 33.481 | −1.800 | 50.165 | 1.00 | 25.48 | B |
| ATOM | 558 | CG1 | VAL | 85 | 32.566 | −1.623 | 51.354 | 1.00 | 24.98 | B |
| ATOM | 559 | CG2 | VAL | 85 | 34.514 | −2.865 | 50.448 | 1.00 | 26.13 | B |
| ATOM | 560 | C | VAL | 85 | 31.684 | −1.024 | 48.613 | 1.00 | 25.90 | B |
| ATOM | 561 | O | VAL | 85 | 30.480 | −1.167 | 48.779 | 1.00 | 24.94 | B |
| ATOM | 562 | N | VAL | 86 | 32.205 | 0.106 | 48.152 | 1.00 | 26.94 | B |
| ATOM | 563 | CA | VAL | 86 | 31.368 | 1.281 | 47.916 | 1.00 | 27.62 | B |
| ATOM | 564 | CB | VAL | 86 | 32.227 | 2.551 | 47.793 | 1.00 | 25.49 | B |
| ATOM | 565 | CG1 | VAL | 86 | 31.384 | 3.763 | 48.096 | 1.00 | 25.95 | B |
| ATOM | 566 | CG2 | VAL | 86 | 33.418 | 2.480 | 48.722 | 1.00 | 24.40 | B |
| ATOM | 567 | C | VAL | 86 | 30.395 | 1.267 | 46.736 | 1.00 | 28.91 | B |
| ATOM | 568 | O | VAL | 86 | 29.254 | 1.709 | 46.874 | 1.00 | 27.52 | B |
| ATOM | 569 | N | CYS | 87 | 30.835 | 0.773 | 45.583 | 1.00 | 30.20 | B |
| ATOM | 570 | CA | CYS | 87 | 29.978 | 0.748 | 44.402 | 1.00 | 31.96 | B |
| ATOM | 571 | CB | CYS | 87 | 30.692 | 0.026 | 43.257 | 1.00 | 35.17 | B |
| ATOM | 572 | SG | CYS | 87 | 30.072 | 0.418 | 41.599 | 1.00 | 41.71 | B |
| ATOM | 573 | C | CYS | 87 | 28.593 | 0.126 | 44.653 | 1.00 | 32.37 | B |
| ATOM | 574 | O | CYS | 87 | 27.571 | 0.682 | 44.234 | 1.00 | 31.48 | B |
| ATOM | 575 | N | PRO | 88 | 28.538 | −1.028 | 45.347 | 1.00 | 31.98 | B |
| ATOM | 576 | CD | PRO | 88 | 29.675 | −1.840 | 45.803 | 1.00 | 32.51 | B |
| ATOM | 577 | CA | PRO | 88 | 27.272 | −1.712 | 45.648 | 1.00 | 30.72 | B |
| ATOM | 578 | CB | PRO | 88 | 27.720 | −3.024 | 46.269 | 1.00 | 31.27 | B |
| ATOM | 579 | CG | PRO | 88 | 29.104 | −3.223 | 45.739 | 1.00 | 32.03 | B |
| ATOM | 580 | C | PRO | 88 | 26.407 | −0.907 | 46.617 | 1.00 | 30.37 | B |
| ATOM | 581 | O | PRO | 88 | 25.179 | −0.928 | 46.528 | 1.00 | 29.46 | B |
| ATOM | 582 | N | ILE | 89 | 27.060 | −0.214 | 47.549 | 1.00 | 28.89 | B |
| ATOM | 583 | CA | ILE | 89 | 26.372 | 0.607 | 48.539 | 1.00 | 26.92 | B |
| ATOM | 584 | CB | ILE | 89 | 27.325 | 1.032 | 49.677 | 1.00 | 27.36 | B |
| ATOM | 585 | CG2 | ILE | 89 | 26.562 | 1.827 | 50.728 | 1.00 | 29.65 | B |
| ATOM | 586 | CG1 | ILE | 89 | 27.949 | −0.202 | 50.327 | 1.00 | 28.47 | B |
| ATOM | 587 | CD1 | ILE | 89 | 28.880 | 0.116 | 51.493 | 1.00 | 28.07 | B |
| ATOM | 588 | C | ILE | 89 | 25.815 | 1.866 | 47.883 | 1.00 | 26.45 | B |
| ATOM | 589 | O | ILE | 89 | 24.733 | 2.329 | 48.236 | 1.00 | 25.57 | B |
| ATOM | 590 | N | LEU | 90 | 26.551 | 2.416 | 46.922 | 1.00 | 26.88 | B |
| ATOM | 591 | CA | LEU | 90 | 26.097 | 3.618 | 46.242 | 1.00 | 27.21 | B |
| ATOM | 592 | CB | LEU | 90 | 27.185 | 4.167 | 45.305 | 1.00 | 26.30 | B |
| ATOM | 593 | CG | LEU | 90 | 26.768 | 5.457 | 44.531 | 1.00 | 28.27 | B |
| ATOM | 594 | CD1 | LEU | 90 | 26.300 | 6.546 | 45.499 | 1.00 | 27.39 | B |
| ATOM | 595 | CD2 | LEU | 90 | 27.936 | 5.952 | 43.707 | 1.00 | 30.13 | B |
| ATOM | 596 | C | LEU | 90 | 24.828 | 3.334 | 45.451 | 1.00 | 28.12 | B |
| ATOM | 597 | O | LEU | 90 | 23.914 | 4.156 | 45.423 | 1.00 | 27.80 | B |
| ATOM | 598 | N | ASP | 91 | 24.778 | 2.168 | 44.811 | 1.00 | 29.04 | B |
| ATOM | 599 | CA | ASP | 91 | 23.615 | 1.782 | 44.029 | 1.00 | 29.68 | B |
| ATOM | 600 | CB | ASP | 91 | 23.888 | 0.479 | 43.238 | 1.00 | 30.25 | B |
| ATOM | 601 | CG | ASP | 91 | 24.715 | 0.717 | 41.975 | 1.00 | 33.21 | B |
| ATOM | 602 | OD1 | ASP | 91 | 24.655 | 1.836 | 41.417 | 1.00 | 33.99 | B |
| ATOM | 603 | OD2 | ASP | 91 | 25.409 | −0.225 | 41.522 | 1.00 | 34.57 | B |
| ATOM | 604 | C | ASP | 91 | 22.412 | 1.604 | 44.950 | 1.00 | 29.79 | B |
| ATOM | 605 | O | ASP | 91 | 21.265 | 1.785 | 44.542 | 1.00 | 29.34 | B |
| ATOM | 606 | N | GLU | 92 | 22.684 | 1.254 | 46.199 | 1.00 | 30.26 | B |
| ATOM | 607 | CA | GLU | 92 | 21.632 | 1.077 | 47.191 | 1.00 | 33.20 | B |
| ATOM | 608 | CB | GLU | 92 | 22.240 | 0.434 | 48.455 | 1.00 | 37.58 | B |
| ATOM | 609 | CG | GLU | 92 | 21.243 | −0.021 | 49.519 | 1.00 | 45.34 | B |
| ATOM | 610 | CD | GLU | 92 | 20.622 | −1.378 | 49.215 | 1.00 | 49.33 | B |
| ATOM | 611 | OE1 | GLU | 92 | 19.996 | −1.963 | 50.134 | 1.00 | 51.49 | B |
| ATOM | 612 | OE2 | GLU | 92 | 20.760 | −1.851 | 48.061 | 1.00 | 50.48 | B |
| ATOM | 613 | C | GLU | 92 | 21.036 | 2.471 | 47.516 | 1.00 | 32.34 | B |
| ATOM | 614 | O | GLU | 92 | 19.816 | 2.659 | 47.548 | 1.00 | 31.40 | B |
| ATOM | 615 | N | VAL | 93 | 21.921 | 3.438 | 47.757 | 1.00 | 29.83 | B |
| ATOM | 616 | CA | VAL | 93 | 21.532 | 4.813 | 48.060 | 1.00 | 27.09 | B |
| ATOM | 617 | CB | VAL | 93 | 22.794 | 5.732 | 48.216 | 1.00 | 27.00 | B |
| ATOM | 618 | CG1 | VAL | 93 | 22.362 | 7.185 | 48.503 | 1.00 | 23.70 | B |
| ATOM | 619 | CG2 | VAL | 93 | 23.720 | 5.189 | 49.320 | 1.00 | 24.02 | B |
| ATOM | 620 | C | VAL | 93 | 20.661 | 5.384 | 46.936 | 1.00 | 25.06 | B |
| ATOM | 621 | O | VAL | 93 | 19.631 | 6.005 | 47.184 | 1.00 | 23.16 | B |
| ATOM | 622 | N | ILE | 94 | 21.090 | 5.173 | 45.700 | 1.00 | 23.81 | B |
| ATOM | 623 | CA | ILE | 94 | 20.357 | 5.679 | 44.554 | 1.00 | 26.20 | B |
| ATOM | 624 | CB | ILE | 94 | 21.196 | 5.496 | 43.268 | 1.00 | 24.09 | B |
| ATOM | 625 | CG2 | ILE | 94 | 20.398 | 5.871 | 42.040 | 1.00 | 22.58 | B |
| ATOM | 626 | CG1 | ILE | 94 | 22.436 | 6.394 | 43.367 | 1.00 | 23.30 | B |
| ATOM | 627 | CD1 | ILE | 94 | 23.378 | 6.288 | 42.211 | 1.00 | 25.19 | B |
| ATOM | 628 | C | ILE | 94 | 18.964 | 5.057 | 44.417 | 1.00 | 28.52 | B |
| ATOM | 629 | O | ILE | 94 | 18.101 | 5.606 | 43.742 | 1.00 | 30.41 | B |
| ATOM | 630 | N | MET | 95 | 18.729 | 3.925 | 45.073 | 1.00 | 31.00 | B |
| ATOM | 631 | CA | MET | 95 | 17.408 | 3.305 | 45.032 | 1.00 | 32.10 | B |
| ATOM | 632 | CB | MET | 95 | 17.501 | 1.789 | 45.171 | 1.00 | 35.87 | B |
| ATOM | 633 | CG | MET | 95 | 17.836 | 1.059 | 43.885 | 1.00 | 39.09 | B |
| ATOM | 634 | SD | MET | 95 | 17.725 | −0.743 | 44.078 | 1.00 | 46.44 | B |
| ATOM | 635 | CE | MET | 95 | 19.451 | −1.155 | 44.567 | 1.00 | 42.73 | B |
| ATOM | 636 | C | MET | 95 | 16.514 | 3.857 | 46.140 | 1.00 | 31.79 | B |
| ATOM | 637 | O | MET | 95 | 15.340 | 3.518 | 46.204 | 1.00 | 32.44 | B |
| ATOM | 638 | N | GLY | 96 | 17.069 | 4.697 | 47.016 | 1.00 | 31.15 | B |
| ATOM | 639 | CA | GLY | 96 | 16.274 | 5.290 | 48.083 | 1.00 | 30.86 | B |
| ATOM | 640 | C | GLY | 96 | 16.506 | 4.778 | 49.497 | 1.00 | 31.33 | B |
| ATOM | 641 | O | GLY | 96 | 15.695 | 5.005 | 50.398 | 1.00 | 31.96 | B |
| ATOM | 642 | N | TYR | 97 | 17.617 | 4.085 | 49.700 | 1.00 | 31.69 | B |
| ATOM | 643 | CA | TYR | 97 | 17.951 | 3.539 | 51.009 | 1.00 | 31.47 | B |
| ATOM | 644 | CB | TYR | 97 | 18.620 | 2.119 | 50.859 | 1.00 | 35.21 | B |
| ATOM | 645 | CG | TYR | 97 | 17.707 | 0.979 | 50.448 | 1.00 | 38.09 | B |
| ATOM | 646 | CD1 | TYR | 97 | 16.856 | 0.369 | 51.374 | 1.00 | 38.78 | B |
| ATOM | 647 | CE1 | TYR | 97 | 16.060 | −0.716 | 51.017 | 1.00 | 39.92 | B |
| ATOM | 648 | CD2 | TYR | 97 | 17.733 | 0.476 | 49.146 | 1.00 | 38.17 | B |
| ATOM | 649 | CE2 | TYR | 97 | 16.938 | −0.606 | 48.777 | 1.00 | 40.59 | B |
| ATOM | 650 | CZ | TYR | 97 | 16.105 | −1.197 | 49.717 | 1.00 | 42.01 | B |
| ATOM | 651 | OH | TYR | 97 | 15.314 | −2.262 | 49.350 | 1.00 | 44.26 | B |
| ATOM | 652 | C | TYR | 97 | 18.944 | 4.465 | 51.699 | 1.00 | 29.27 | B |
| ATOM | 653 | O | TYR | 97 | 19.557 | 5.309 | 51.055 | 1.00 | 29.87 | B |
| ATOM | 654 | N | ASN | 98 | 19.089 | 4.308 | 53.008 | 1.00 | 26.93 | B |
| ATOM | 655 | CA | ASN | 98 | 20.061 | 5.081 | 53.768 | 1.00 | 27.11 | B |
| ATOM | 656 | CB | ASN | 98 | 19.500 | 5.509 | 55.156 | 1.00 | 27.12 | B |
| ATOM | 657 | CG | ASN | 98 | 18.435 | 6.579 | 55.048 | 1.00 | 27.28 | B |
| ATOM | 658 | OD1 | ASN | 98 | 18.553 | 7.506 | 54.245 | 1.00 | 30.11 | B |
| ATOM | 659 | ND2 | ASN | 98 | 17.394 | 6.465 | 55.860 | 1.00 | 26.60 | B |
| ATOM | 660 | C | ASN | 98 | 21.243 | 4.141 | 53.975 | 1.00 | 26.22 | B |
| ATOM | 661 | O | ASN | 98 | 21.055 | 2.971 | 54.292 | 1.00 | 25.58 | B |
| ATOM | 662 | N | CYS | 99 | 22.457 | 4.634 | 53.775 | 1.00 | 25.47 | B |
| ATOM | 663 | CA | CYS | 99 | 23.629 | 3.791 | 53.977 | 1.00 | 25.10 | B |
| ATOM | 664 | CB | CYS | 99 | 24.206 | 3.357 | 52.654 | 1.00 | 26.81 | B |
| ATOM | 665 | SG | CYS | 99 | 23.084 | 2.317 | 51.714 | 1.00 | 26.81 | B |
| ATOM | 666 | C | CYS | 99 | 24.697 | 4.486 | 54.798 | 1.00 | 23.75 | B |
| ATOM | 667 | O | CYS | 99 | 24.804 | 5.712 | 54.804 | 1.00 | 25.67 | B |
| ATOM | 668 | N | THR | 100 | 25.482 | 3.683 | 55.496 | 1.00 | 20.94 | B |
| ATOM | 669 | CA | THR | 100 | 26.549 | 4.181 | 56.341 | 1.00 | 19.27 | B |
| ATOM | 670 | CB | THR | 100 | 26.076 | 4.266 | 57.795 | 1.00 | 17.86 | B |
| ATOM | 671 | OG1 | THR | 100 | 24.992 | 5.192 | 57.875 | 1.00 | 16.90 | B |
| ATOM | 672 | CG2 | THR | 100 | 27.202 | 4.714 | 58.708 | 1.00 | 17.10 | B |
| ATOM | 673 | C | THR | 100 | 27.760 | 3.247 | 56.269 | 1.00 | 19.78 | B |
| ATOM | 674 | O | THR | 100 | 27.615 | 2.013 | 56.297 | 1.00 | 19.41 | B |
| ATOM | 675 | N | ILE | 101 | 28.945 | 3.846 | 56.170 | 1.00 | 17.12 | B |
| ATOM | 676 | CA | ILE | 101 | 30.194 | 3.096 | 56.112 | 1.00 | 13.84 | B |
| ATOM | 677 | CB | ILE | 101 | 30.923 | 3.273 | 54.770 | 1.00 | 11.63 | B |
| ATOM | 678 | CG2 | ILE | 101 | 32.193 | 2.459 | 54.763 | 1.00 | 11.54 | B |
| ATOM | 679 | CG1 | ILE | 101 | 30.029 | 2.847 | 53.614 | 1.00 | 11.12 | B |
| ATOM | 680 | CD1 | ILE | 101 | 30.610 | 3.205 | 52.240 | 1.00 | 8.60 | B |
| ATOM | 681 | C | ILE | 101 | 31.088 | 3.655 | 57.189 | 1.00 | 14.61 | B |
| ATOM | 682 | O | ILE | 101 | 31.434 | 4.828 | 57.158 | 1.00 | 16.06 | B |
| ATOM | 683 | N | PHE | 102 | 31.454 | 2.814 | 58.149 | 1.00 | 16.69 | B |
| ATOM | 684 | CA | PHE | 102 | 32.336 | 3.214 | 59.246 | 1.00 | 15.45 | B |
| ATOM | 685 | CB | PHE | 102 | 31.957 | 2.509 | 60.517 | 1.00 | 15.38 | B |
| ATOM | 686 | CG | PHE | 102 | 30.704 | 3.002 | 61.158 | 1.00 | 17.02 | B |
| ATOM | 687 | CD1 | PHE | 102 | 30.746 | 4.068 | 62.060 | 1.00 | 14.70 | B |
| ATOM | 688 | CD2 | PHE | 102 | 29.489 | 2.341 | 60.937 | 1.00 | 15.06 | B |
| ATOM | 689 | CE1 | PHE | 102 | 29.601 | 4.468 | 62.744 | 1.00 | 15.17 | B |
| ATOM | 690 | CE2 | PHE | 102 | 28.336 | 2.732 | 61.614 | 1.00 | 16.46 | B |
| ATOM | 691 | CZ | PHE | 102 | 28.389 | 3.797 | 62.523 | 1.00 | 16.06 | B |
| ATOM | 692 | C | PHE | 102 | 33.770 | 2.789 | 58.956 | 1.00 | 13.66 | B |
| ATOM | 693 | O | PHE | 102 | 34.004 | 1.767 | 58.335 | 1.00 | 14.29 | B |
| ATOM | 694 | N | ALA | 103 | 34.723 | 3.571 | 59.431 | 1.00 | 14.00 | B |
| ATOM | 695 | CA | ALA | 103 | 36.135 | 3.230 | 59.309 | 1.00 | 13.68 | B |
| ATOM | 696 | CB | ALA | 103 | 36.894 | 4.316 | 58.595 | 1.00 | 12.73 | B |
| ATOM | 697 | C | ALA | 103 | 36.579 | 3.142 | 60.771 | 1.00 | 14.68 | B |
| ATOM | 698 | O | ALA | 103 | 36.560 | 4.144 | 61.491 | 1.00 | 12.81 | B |
| ATOM | 699 | N | TYR | 104 | 36.943 | 1.939 | 61.211 | 1.00 | 14.23 | B |
| ATOM | 700 | CA | TYR | 104 | 37.369 | 1.722 | 62.588 | 1.00 | 13.28 | B |
| ATOM | 701 | CB | TYR | 104 | 36.415 | 0.741 | 63.271 | 1.00 | 13.08 | B |
| ATOM | 702 | CG | TYR | 104 | 36.704 | 0.496 | 64.740 | 1.00 | 9.23 | B |
| ATOM | 703 | CD1 | TYR | 104 | 37.774 | −0.304 | 65.139 | 1.00 | 10.77 | B |
| ATOM | 704 | CE1 | TYR | 104 | 38.050 | −0.519 | 66.497 | 1.00 | 8.87 | B |
| ATOM | 705 | CD2 | TYR | 104 | 35.916 | 1.072 | 65.728 | 1.00 | 7.28 | B |
| ATOM | 706 | CE2 | TYR | 104 | 36.180 | 0.861 | 67.085 | 1.00 | 6.26 | B |
| ATOM | 707 | CZ | TYR | 104 | 37.245 | 0.063 | 67.459 | 1.00 | 6.63 | B |
| ATOM | 708 | OH | TYR | 104 | 37.492 | −0.189 | 68.791 | 1.00 | 6.91 | B |
| ATOM | 709 | C | TYR | 104 | 38.791 | 1.191 | 62.660 | 1.00 | 14.55 | B |
| ATOM | 710 | O | TYR | 104 | 39.192 | 0.344 | 61.866 | 1.00 | 17.36 | B |
| ATOM | 711 | N | GLY | 105 | 39.553 | 1.688 | 63.622 | 1.00 | 15.00 | B |
| ATOM | 712 | CA | GLY | 105 | 40.920 | 1.239 | 63.760 | 1.00 | 16.15 | B |
| ATOM | 713 | C | GLY | 105 | 41.818 | 2.222 | 64.480 | 1.00 | 18.48 | B |
| ATOM | 714 | O | GLY | 105 | 41.464 | 3.383 | 64.733 | 1.00 | 19.06 | B |
| ATOM | 715 | N | GLN | 106 | 42.996 | 1.726 | 64.818 | 1.00 | 18.69 | B |
| ATOM | 716 | CA | GLN | 106 | 44.012 | 2.480 | 65.524 | 1.00 | 20.40 | B |
| ATOM | 717 | CB | GLN | 106 | 45.109 | 1.510 | 65.958 | 1.00 | 20.92 | B |
| ATOM | 718 | CG | GLN | 106 | 46.494 | 2.093 | 65.959 | 1.00 | 25.11 | B |
| ATOM | 719 | CD | GLN | 106 | 47.546 | 1.104 | 66.424 | 1.00 | 27.12 | B |
| ATOM | 720 | OE1 | GLN | 106 | 47.724 | 0.033 | 65.833 | 1.00 | 29.47 | B |
| ATOM | 721 | NE2 | GLN | 106 | 48.254 | 1.462 | 67.486 | 1.00 | 24.05 | B |
| ATOM | 722 | C | GLN | 106 | 44.595 | 3.602 | 64.668 | 1.00 | 22.74 | B |
| ATOM | 723 | O | GLN | 106 | 44.733 | 3.442 | 63.447 | 1.00 | 22.56 | B |
| ATOM | 724 | N | THR | 107 | 44.924 | 4.733 | 65.312 | 1.00 | 22.64 | B |
| ATOM | 725 | CA | THR | 107 | 45.526 | 5.893 | 64.637 | 1.00 | 21.79 | B |
| ATOM | 726 | CB | THR | 107 | 46.070 | 6.943 | 65.659 | 1.00 | 22.17 | B |
| ATOM | 727 | OG1 | THR | 107 | 45.014 | 7.404 | 66.510 | 1.00 | 22.36 | B |
| ATOM | 728 | CG2 | THR | 107 | 46.675 | 8.142 | 64.927 | 1.00 | 19.97 | B |
| ATOM | 729 | C | THR | 107 | 46.720 | 5.430 | 63.788 | 1.00 | 21.90 | B |
| ATOM | 730 | O | THR | 107 | 47.605 | 4.752 | 64.288 | 1.00 | 20.99 | B |
| ATOM | 731 | N | GLY | 108 | 46.739 | 5.796 | 62.510 | 1.00 | 22.46 | B |
| ATOM | 732 | CA | GLY | 108 | 47.836 | 5.394 | 61.652 | 1.00 | 21.62 | B |
| ATOM | 733 | C | GLY | 108 | 47.664 | 4.088 | 60.882 | 1.00 | 22.90 | B |
| ATOM | 734 | O | GLY | 108 | 48.653 | 3.547 | 60.376 | 1.00 | 24.07 | B |
| ATOM | 735 | N | THR | 109 | 46.436 | 3.572 | 60.786 | 1.00 | 22.29 | B |
| ATOM | 736 | CA | THR | 109 | 46.197 | 2.321 | 60.050 | 1.00 | 21.18 | B |
| ATOM | 737 | CB | THR | 109 | 45.408 | 1.259 | 60.884 | 1.00 | 21.26 | B |
| ATOM | 738 | OG1 | THR | 109 | 44.159 | 1.814 | 61.335 | 1.00 | 20.11 | B |
| ATOM | 739 | CG2 | THR | 109 | 46.250 | 0.777 | 62.071 | 1.00 | 19.60 | B |
| ATOM | 740 | C | THR | 109 | 45.439 | 2.523 | 58.754 | 1.00 | 19.58 | B |
| ATOM | 741 | O | THR | 109 | 45.126 | 1.551 | 58.068 | 1.00 | 20.97 | B |
| ATOM | 742 | N | GLY | 110 | 45.125 | 3.776 | 58.428 | 1.00 | 17.22 | B |
| ATOM | 743 | CA | GLY | 110 | 44.415 | 4.048 | 57.193 | 1.00 | 12.69 | B |
| ATOM | 744 | C | GLY | 110 | 42.943 | 4.424 | 57.232 | 1.00 | 12.29 | B |
| ATOM | 745 | O | GLY | 110 | 42.288 | 4.365 | 56.193 | 1.00 | 14.37 | B |
| ATOM | 746 | N | LYS | 111 | 42.398 | 4.795 | 58.386 | 1.00 | 11.41 | B |
| ATOM | 747 | CA | LYS | 111 | 40.983 | 5.198 | 58.432 | 1.00 | 12.47 | B |
| ATOM | 748 | CB | LYS | 111 | 40.540 | 5.653 | 59.898 | 1.00 | 13.24 | B |
| ATOM | 749 | CG | LYS | 111 | 40.379 | 4.538 | 60.934 | 1.00 | 10.82 | B |
| ATOM | 750 | CD | LYS | 111 | 39.805 | 5.061 | 62.229 | 1.00 | 6.09 | B |
| ATOM | 751 | CE | LYS | 111 | 40.691 | 6.142 | 62.813 | 1.00 | 10.33 | B |
| ATOM | 752 | NZ | LYS | 111 | 42.130 | 5.748 | 63.038 | 1.00 | 9.60 | B |
| ATOM | 753 | C | LYS | 111 | 40.742 | 6.363 | 57.465 | 1.00 | 13.44 | B |
| ATOM | 754 | O | LYS | 111 | 39.870 | 6.295 | 56.587 | 1.00 | 14.48 | B |
| ATOM | 755 | N | THR | 112 | 41.538 | 7.423 | 57.614 | 1.00 | 14.82 | B |
| ATOM | 756 | CA | THR | 112 | 41.403 | 8.613 | 56.773 | 1.00 | 15.93 | B |
| ATOM | 757 | CB | THR | 112 | 42.140 | 9.793 | 57.417 | 1.00 | 15.93 | B |
| ATOM | 758 | OG1 | THR | 112 | 41.538 | 10.066 | 58.694 | 1.00 | 14.63 | B |
| ATOM | 759 | CG2 | THR | 112 | 42.055 | 11.040 | 56.522 | 1.00 | 13.41 | B |
| ATOM | 760 | C | THR | 112 | 41.870 | 8.426 | 55.323 | 1.00 | 17.21 | B |
| ATOM | 761 | O | THR | 112 | 41.318 | 9.021 | 54.385 | 1.00 | 16.82 | B |
| ATOM | 762 | N | PHE | 113 | 42.887 | 7.595 | 55.142 | 1.00 | 17.40 | B |
| ATOM | 763 | CA | PHE | 113 | 43.398 | 7.313 | 53.811 | 1.00 | 16.82 | B |
| ATOM | 764 | CB | PHE | 113 | 44.654 | 6.389 | 53.889 | 1.00 | 16.02 | B |
| ATOM | 765 | CG | PHE | 113 | 45.233 | 6.054 | 52.540 | 1.00 | 17.10 | B |
| ATOM | 766 | CD1 | PHE | 113 | 46.126 | 6.918 | 51.920 | 1.00 | 18.15 | B |
| ATOM | 767 | CD2 | PHE | 113 | 44.836 | 4.911 | 51.868 | 1.00 | 18.15 | B |
| ATOM | 768 | CE1 | PHE | 113 | 46.614 | 6.654 | 50.652 | 1.00 | 19.37 | B |
| ATOM | 769 | CE2 | PHE | 113 | 45.317 | 4.632 | 50.588 | 1.00 | 20.77 | B |
| ATOM | 770 | CZ | PHE | 113 | 46.208 | 5.508 | 49.980 | 1.00 | 21.58 | B |
| ATOM | 771 | C | PHE | 113 | 42.305 | 6.615 | 52.997 | 1.00 | 15.35 | B |
| ATOM | 772 | O | PHE | 113 | 42.125 | 6.894 | 51.816 | 1.00 | 13.50 | B |
| ATOM | 773 | N | THR | 114 | 41.590 | 5.700 | 53.647 | 1.00 | 14.49 | B |
| ATOM | 774 | CA | THR | 114 | 40.524 | 4.942 | 53.008 | 1.00 | 13.72 | B |
| ATOM | 775 | CB | THR | 114 | 40.119 | 3.722 | 53.868 | 1.00 | 14.47 | B |
| ATOM | 776 | OG1 | THR | 114 | 41.228 | 2.834 | 53.980 | 1.00 | 13.50 | B |
| ATOM | 777 | CG2 | THR | 114 | 38.944 | 2.984 | 53.258 | 1.00 | 10.99 | B |
| ATOM | 778 | C | THR | 114 | 39.283 | 5.773 | 52.764 | 1.00 | 13.62 | B |
| ATOM | 779 | O | THR | 114 | 38.733 | 5.758 | 51.674 | 1.00 | 14.61 | B |
| ATOM | 780 | N | MET | 115 | 38.842 | 6.499 | 53.784 | 1.00 | 15.54 | B |
| ATOM | 781 | CA | MET | 115 | 37.635 | 7.311 | 53.663 | 1.00 | 16.98 | B |
| ATOM | 782 | CB | MET | 115 | 37.121 | 7.711 | 55.043 | 1.00 | 17.73 | B |
| ATOM | 783 | CG | MET | 115 | 36.776 | 6.525 | 55.938 | 1.00 | 22.32 | B |
| ATOM | 784 | SD | MET | 115 | 35.694 | 5.280 | 55.139 | 1.00 | 24.33 | B |
| ATOM | 785 | CE | MET | 115 | 34.110 | 6.102 | 55.162 | 1.00 | 17.96 | B |
| ATOM | 786 | C | MET | 115 | 37.772 | 8.556 | 52.809 | 1.00 | 16.94 | B |
| ATOM | 787 | O | MET | 115 | 36.824 | 8.956 | 52.140 | 1.00 | 17.35 | B |
| ATOM | 788 | N | GLU | 116 | 38.947 | 9.168 | 52.816 | 1.00 | 16.96 | B |
| ATOM | 789 | CA | GLU | 116 | 39.139 | 10.391 | 52.040 | 1.00 | 17.40 | B |
| ATOM | 790 | CB | GLU | 116 | 39.564 | 11.563 | 52.988 | 1.00 | 17.75 | B |
| ATOM | 791 | CG | GLU | 116 | 38.457 | 12.038 | 53.929 | 1.00 | 20.71 | B |
| ATOM | 792 | CD | GLU | 116 | 38.980 | 12.893 | 55.070 | 1.00 | 22.10 | B |
| ATOM | 793 | OE1 | GLU | 116 | 40.113 | 13.404 | 54.961 | 1.00 | 26.78 | B |
| ATOM | 794 | OE2 | GLU | 116 | 38.260 | 13.064 | 56.074 | 1.00 | 22.44 | B |
| ATOM | 795 | C | GLU | 116 | 40.178 | 10.211 | 50.953 | 1.00 | 16.14 | B |
| ATOM | 796 | O | GLU | 116 | 39.925 | 10.474 | 49.783 | 1.00 | 12.66 | B |
| ATOM | 797 | N | GLY | 117 | 41.357 | 9.768 | 51.360 | 1.00 | 16.93 | B |
| ATOM | 798 | CA | GLY | 117 | 42.425 | 9.585 | 50.406 | 1.00 | 21.10 | B |
| ATOM | 799 | C | GLY | 117 | 43.424 | 10.723 | 50.439 | 1.00 | 22.08 | B |
| ATOM | 800 | O | GLY | 117 | 43.321 | 11.640 | 51.248 | 1.00 | 21.52 | B |
| ATOM | 801 | N | GLU | 118 | 44.390 | 10.661 | 49.536 | 1.00 | 24.00 | B |
| ATOM | 802 | CA | GLU | 118 | 45.436 | 11.664 | 49.457 | 1.00 | 26.12 | B |
| ATOM | 803 | CB | GLU | 118 | 46.712 | 11.116 | 50.134 | 1.00 | 27.39 | B |
| ATOM | 804 | CG | GLU | 118 | 46.574 | 11.023 | 51.647 | 1.00 | 32.78 | B |
| ATOM | 805 | CD | GLU | 118 | 47.603 | 10.111 | 52.316 | 1.00 | 37.03 | B |
| ATOM | 806 | OE1 | GLU | 118 | 48.799 | 10.149 | 51.938 | 1.00 | 36.38 | B |
| ATOM | 807 | OE2 | GLU | 118 | 47.208 | 9.369 | 53.246 | 1.00 | 39.57 | B |
| ATOM | 808 | C | GLU | 118 | 45.702 | 12.026 | 48.000 | 1.00 | 26.11 | B |
| ATOM | 809 | O | GLU | 118 | 45.079 | 11.481 | 47.088 | 1.00 | 24.83 | B |
| ATOM | 810 | N | ARG | 119 | 46.613 | 12.961 | 47.780 | 1.00 | 25.93 | B |
| ATOM | 811 | CA | ARG | 119 | 46.922 | 13.355 | 46.423 | 1.00 | 26.49 | B |
| ATOM | 812 | CB | ARG | 119 | 47.076 | 14.913 | 46.313 | 1.00 | 24.19 | B |
| ATOM | 813 | CG | ARG | 119 | 45.824 | 15.737 | 46.642 | 1.00 | 18.83 | B |
| ATOM | 814 | CD | ARG | 119 | 44.579 | 15.206 | 45.965 | 1.00 | 15.06 | B |
| ATOM | 815 | NE | ARG | 119 | 44.755 | 14.940 | 44.542 | 1.00 | 15.80 | B |
| ATOM | 816 | CZ | ARG | 119 | 44.761 | 15.869 | 43.591 | 1.00 | 18.90 | B |
| ATOM | 817 | NH1 | ARG | 119 | 44.601 | 17.142 | 43.910 | 1.00 | 20.61 | B |
| ATOM | 818 | NH2 | ARG | 119 | 44.910 | 15.528 | 42.314 | 1.00 | 17.87 | B |
| ATOM | 819 | C | ARG | 119 | 48.207 | 12.682 | 45.967 | 1.00 | 29.08 | B |
| ATOM | 820 | O | ARG | 119 | 49.178 | 12.572 | 46.735 | 1.00 | 27.84 | B |
| ATOM | 821 | N | SER | 120 | 48.205 | 12.192 | 44.731 | 1.00 | 30.37 | B |
| ATOM | 822 | CA | SER | 120 | 49.417 | 11.597 | 44.203 | 1.00 | 32.15 | B |
| ATOM | 823 | CB | SER | 120 | 49.190 | 11.014 | 42.825 | 1.00 | 33.55 | B |
| ATOM | 824 | OG | SER | 120 | 48.380 | 9.854 | 42.897 | 1.00 | 34.65 | B |
| ATOM | 825 | C | SER | 120 | 50.287 | 12.839 | 44.123 | 1.00 | 31.39 | B |
| ATOM | 826 | O | SER | 120 | 49.849 | 13.883 | 43.651 | 1.00 | 31.19 | B |
| ATOM | 827 | N | PRO | 121 | 51.522 | 12.745 | 44.599 | 1.00 | 30.67 | B |
| ATOM | 828 | CD | PRO | 121 | 52.207 | 11.494 | 44.965 | 1.00 | 31.67 | B |
| ATOM | 829 | CA | PRO | 121 | 52.455 | 13.870 | 44.595 | 1.00 | 31.71 | B |
| ATOM | 830 | CB | PRO | 121 | 53.674 | 13.288 | 45.270 | 1.00 | 31.87 | B |
| ATOM | 831 | CG | PRO | 121 | 53.658 | 11.869 | 44.783 | 1.00 | 32.88 | B |
| ATOM | 832 | C | PRO | 121 | 52.788 | 14.511 | 43.240 | 1.00 | 32.30 | B |
| ATOM | 833 | O | PRO | 121 | 52.557 | 13.925 | 42.176 | 1.00 | 32.32 | B |
| ATOM | 834 | N | ASN | 122 | 53.319 | 15.733 | 43.319 | 1.00 | 30.43 | B |
| ATOM | 835 | CA | ASN | 122 | 53.753 | 16.529 | 42.175 | 1.00 | 30.58 | B |
| ATOM | 836 | CB | ASN | 122 | 54.974 | 15.864 | 41.515 | 1.00 | 30.83 | B |
| ATOM | 837 | CG | ASN | 122 | 56.101 | 16.850 | 41.250 | 1.00 | 29.55 | B |
| ATOM | 838 | OD1 | ASN | 122 | 56.512 | 17.589 | 42.139 | 1.00 | 30.20 | B |
| ATOM | 839 | ND2 | ASN | 122 | 56.614 | 16.849 | 40.032 | 1.00 | 29.25 | B |
| ATOM | 840 | C | ASN | 122 | 52.708 | 16.838 | 41.107 | 1.00 | 30.96 | B |
| ATOM | 841 | O | ASN | 122 | 53.022 | 16.840 | 39.916 | 1.00 | 28.89 | B |
| ATOM | 842 | N | GLU | 123 | 51.479 | 17.121 | 41.540 | 1.00 | 31.29 | B |
| ATOM | 843 | CA | GLU | 123 | 50.380 | 17.435 | 40.630 | 1.00 | 31.61 | B |
| ATOM | 844 | CB | GLU | 123 | 50.437 | 18.873 | 40.222 | 1.00 | 29.75 | B |
| ATOM | 845 | CG | GLU | 123 | 50.311 | 19.825 | 41.382 | 1.00 | 31.53 | B |
| ATOM | 846 | CD | GLU | 123 | 50.030 | 21.243 | 40.942 | 1.00 | 34.00 | B |
| ATOM | 847 | OE1 | GLU | 123 | 50.896 | 21.842 | 40.255 | 1.00 | 32.81 | B |
| ATOM | 848 | OE2 | GLU | 123 | 48.937 | 21.753 | 41.288 | 1.00 | 35.74 | B |
| ATOM | 849 | C | GLU | 123 | 50.396 | 16.558 | 39.393 | 1.00 | 32.07 | B |
| ATOM | 850 | O | GLU | 123 | 50.246 | 17.038 | 38.272 | 1.00 | 32.39 | B |
| ATOM | 851 | N | GLU | 124 | 50.576 | 15.261 | 39.620 | 1.00 | 33.92 | B |
| ATOM | 852 | CA | GLU | 124 | 50.628 | 14.269 | 38.558 | 1.00 | 33.69 | B |
| ATOM | 853 | CB | GLU | 124 | 51.235 | 12.998 | 39.111 | 1.00 | 35.39 | B |
| ATOM | 854 | CG | GLU | 124 | 51.234 | 11.798 | 38.184 | 1.00 | 39.45 | B |
| ATOM | 855 | CD | GLU | 124 | 51.966 | 10.613 | 38.801 | 1.00 | 42.18 | B |
| ATOM | 856 | OE1 | GLU | 124 | 51.802 | 10.390 | 40.026 | 1.00 | 42.52 | B |
| ATOM | 857 | OE2 | GLU | 124 | 52.698 | 9.906 | 38.067 | 1.00 | 42.46 | B |
| ATOM | 858 | C | GLU | 124 | 49.252 | 13.994 | 37.958 | 1.00 | 33.48 | B |
| ATOM | 859 | O | GLU | 124 | 49.149 | 13.665 | 36.778 | 1.00 | 33.85 | B |
| ATOM | 860 | N | TYR | 125 | 48.196 | 14.141 | 38.758 | 1.00 | 32.64 | B |
| ATOM | 861 | CA | TYR | 125 | 46.841 | 13.895 | 38.267 | 1.00 | 33.52 | B |
| ATOM | 862 | CB | TYR | 125 | 46.261 | 12.523 | 38.817 | 1.00 | 33.48 | B |
| ATOM | 863 | CG | TYR | 125 | 47.109 | 11.290 | 38.613 | 1.00 | 35.23 | B |
| ATOM | 864 | CD1 | TYR | 125 | 47.951 | 10.826 | 39.624 | 1.00 | 35.75 | B |
| ATOM | 865 | CE1 | TYR | 125 | 48.709 | 9.668 | 39.461 | 1.00 | 36.41 | B |
| ATOM | 866 | CD2 | TYR | 125 | 47.046 | 10.565 | 37.422 | 1.00 | 36.88 | B |
| ATOM | 867 | CE2 | TYR | 125 | 47.803 | 9.403 | 37.242 | 1.00 | 37.22 | B |
| ATOM | 868 | CZ | TYR | 125 | 48.630 | 8.962 | 38.268 | 1.00 | 38.72 | B |
| ATOM | 869 | OH | TYR | 125 | 49.369 | 7.811 | 38.108 | 1.00 | 40.27 | B |
| ATOM | 870 | C | TYR | 125 | 45.851 | 14.985 | 38.677 | 1.00 | 33.79 | B |
| ATOM | 871 | O | TYR | 125 | 46.150 | 15.834 | 39.520 | 1.00 | 34.63 | B |
| ATOM | 872 | N | THR | 126 | 44.669 | 14.949 | 38.063 | 1.00 | 33.04 | B |
| ATOM | 873 | CA | THR | 126 | 43.588 | 15.858 | 38.420 | 1.00 | 31.85 | B |
| ATOM | 874 | CB | THR | 126 | 42.562 | 16.061 | 37.286 | 1.00 | 31.42 | B |
| ATOM | 875 | OG1 | THR | 126 | 42.214 | 14.790 | 36.723 | 1.00 | 29.37 | B |
| ATOM | 876 | CG2 | THR | 126 | 43.114 | 16.996 | 36.216 | 1.00 | 30.94 | B |
| ATOM | 877 | C | THR | 126 | 42.911 | 15.061 | 39.518 | 1.00 | 31.76 | B |
| ATOM | 878 | O | THR | 126 | 43.023 | 13.836 | 39.552 | 1.00 | 31.47 | B |
| ATOM | 879 | N | TRP | 127 | 42.197 | 15.738 | 40.401 | 1.00 | 31.44 | B |
| ATOM | 880 | CA | TRP | 127 | 41.559 | 15.053 | 41.507 | 1.00 | 30.17 | B |
| ATOM | 881 | CB | TRP | 127 | 40.749 | 16.048 | 42.357 | 1.00 | 27.67 | B |
| ATOM | 882 | CG | TRP | 127 | 39.474 | 16.455 | 41.718 | 1.00 | 25.01 | B |
| ATOM | 883 | CD2 | TRP | 127 | 38.207 | 15.796 | 41.846 | 1.00 | 24.45 | B |
| ATOM | 884 | CE2 | TRP | 127 | 37.285 | 16.514 | 41.059 | 1.00 | 24.12 | B |
| ATOM | 885 | CE3 | TRP | 127 | 37.764 | 14.662 | 42.546 | 1.00 | 22.04 | B |
| ATOM | 886 | CD1 | TRP | 127 | 39.278 | 17.507 | 40.885 | 1.00 | 23.64 | B |
| ATOM | 887 | NE1 | TRP | 127 | 37.966 | 17.553 | 40.483 | 1.00 | 24.14 | B |
| ATOM | 888 | CZ2 | TRP | 127 | 35.937 | 16.143 | 40.952 | 1.00 | 25.81 | B |
| ATOM | 889 | CZ3 | TRP | 127 | 36.427 | 14.285 | 42.441 | 1.00 | 24.07 | B |
| ATOM | 890 | CH2 | TRP | 127 | 35.526 | 15.026 | 41.647 | 1.00 | 26.19 | B |
| ATOM | 891 | C | TRP | 127 | 40.664 | 13.883 | 41.099 | 1.00 | 30.31 | B |
| ATOM | 892 | O | TRP | 127 | 40.635 | 12.859 | 41.784 | 1.00 | 31.25 | B |
| ATOM | 893 | N | GLU | 128 | 39.945 | 14.014 | 39.991 | 1.00 | 30.25 | B |
| ATOM | 894 | CA | GLU | 128 | 39.036 | 12.943 | 39.575 | 1.00 | 29.93 | B |
| ATOM | 895 | CB | GLU | 128 | 38.010 | 13.477 | 38.601 | 1.00 | 30.66 | B |
| ATOM | 896 | CG | GLU | 128 | 38.597 | 14.116 | 37.360 | 1.00 | 32.82 | B |
| ATOM | 897 | CD | GLU | 128 | 37.522 | 14.757 | 36.522 | 1.00 | 37.02 | B |
| ATOM | 898 | OE1 | GLU | 128 | 36.740 | 15.558 | 37.085 | 1.00 | 37.94 | B |
| ATOM | 899 | OE2 | GLU | 128 | 37.450 | 14.460 | 35.309 | 1.00 | 39.71 | B |
| ATOM | 900 | C | GLU | 128 | 39.692 | 11.704 | 38.977 | 1.00 | 28.41 | B |
| ATOM | 901 | O | GLU | 128 | 39.004 | 10.755 | 38.623 | 1.00 | 28.40 | B |
| ATOM | 902 | N | GLU | 129 | 41.012 | 11.716 | 38.853 | 1.00 | 27.73 | B |
| ATOM | 903 | CA | GLU | 129 | 41.724 | 10.574 | 38.303 | 1.00 | 26.98 | B |
| ATOM | 904 | CB | GLU | 129 | 42.343 | 10.919 | 36.940 | 1.00 | 25.80 | B |
| ATOM | 905 | CG | GLU | 129 | 41.317 | 11.144 | 35.841 | 1.00 | 28.03 | B |
| ATOM | 906 | CD | GLU | 129 | 41.954 | 11.422 | 34.487 | 1.00 | 33.17 | B |
| ATOM | 907 | OE1 | GLU | 129 | 41.201 | 11.654 | 33.510 | 1.00 | 35.80 | B |
| ATOM | 908 | OE2 | GLU | 129 | 43.206 | 11.411 | 34.389 | 1.00 | 33.91 | B |
| ATOM | 909 | C | GLU | 129 | 42.807 | 10.110 | 39.257 | 1.00 | 27.19 | B |
| ATOM | 910 | O | GLU | 129 | 43.480 | 9.117 | 38.997 | 1.00 | 28.14 | B |
| ATOM | 911 | N | ASP | 130 | 42.966 | 10.814 | 40.372 | 1.00 | 27.13 | B |
| ATOM | 912 | CA | ASP | 130 | 43.995 | 10.445 | 41.336 | 1.00 | 28.16 | B |
| ATOM | 913 | CB | ASP | 130 | 44.092 | 11.498 | 42.458 | 1.00 | 29.19 | B |
| ATOM | 914 | CG | ASP | 130 | 45.484 | 11.577 | 43.061 | 1.00 | 31.28 | B |
| ATOM | 915 | OD1 | ASP | 130 | 46.026 | 10.525 | 43.470 | 1.00 | 31.52 | B |
| ATOM | 916 | OD2 | ASP | 130 | 46.039 | 12.695 | 43.125 | 1.00 | 33.01 | B |
| ATOM | 917 | C | ASP | 130 | 43.690 | 9.068 | 41.925 | 1.00 | 27.22 | B |
| ATOM | 918 | O | ASP | 130 | 42.646 | 8.865 | 42.551 | 1.00 | 27.12 | B |
| ATOM | 919 | N | PRO | 131 | 44.590 | 8.093 | 41.704 | 1.00 | 26.27 | B |
| ATOM | 920 | CD | PRO | 131 | 45.722 | 8.143 | 40.760 | 1.00 | 25.74 | B |
| ATOM | 921 | CA | PRO | 131 | 44.404 | 6.733 | 42.217 | 1.00 | 25.42 | B |
| ATOM | 922 | CB | PRO | 131 | 45.436 | 5.928 | 41.431 | 1.00 | 25.20 | B |
| ATOM | 923 | CG | PRO | 131 | 46.516 | 6.926 | 41.158 | 1.00 | 25.28 | B |
| ATOM | 924 | C | PRO | 131 | 44.550 | 6.586 | 43.734 | 1.00 | 25.10 | B |
| ATOM | 925 | O | PRO | 131 | 44.317 | 5.514 | 44.284 | 1.00 | 25.70 | B |
| ATOM | 926 | N | LEU | 132 | 44.939 | 7.659 | 44.414 | 1.00 | 25.55 | B |
| ATOM | 927 | CA | LEU | 132 | 45.061 | 7.615 | 45.870 | 1.00 | 24.12 | B |
| ATOM | 928 | CB | LEU | 132 | 46.335 | 8.393 | 46.358 | 1.00 | 23.33 | B |
| ATOM | 929 | CG | LEU | 132 | 47.750 | 7.835 | 45.985 | 1.00 | 24.01 | B |
| ATOM | 930 | CD1 | LEU | 132 | 48.853 | 8.699 | 46.613 | 1.00 | 21.35 | B |
| ATOM | 931 | CD2 | LEU | 132 | 47.875 | 6.394 | 46.474 | 1.00 | 25.49 | B |
| ATOM | 932 | C | LEU | 132 | 43.794 | 8.216 | 46.497 | 1.00 | 23.99 | B |
| ATOM | 933 | O | LEU | 132 | 43.694 | 8.338 | 47.728 | 1.00 | 24.50 | B |
| ATOM | 934 | N | ALA | 133 | 42.831 | 8.587 | 45.650 | 1.00 | 21.97 | B |
| ATOM | 935 | CA | ALA | 133 | 41.566 | 9.155 | 46.129 | 1.00 | 23.50 | B |
| ATOM | 936 | CB | ALA | 133 | 40.738 | 9.710 | 44.958 | 1.00 | 19.96 | B |
| ATOM | 937 | C | ALA | 133 | 40.760 | 8.097 | 46.896 | 1.00 | 24.12 | B |
| ATOM | 938 | O | ALA | 133 | 40.766 | 6.914 | 46.552 | 1.00 | 24.63 | B |
| ATOM | 939 | N | GLY | 134 | 40.060 | 8.546 | 47.931 | 1.00 | 25.21 | B |
| ATOM | 940 | CA | GLY | 134 | 39.289 | 7.646 | 48.763 | 1.00 | 23.61 | B |
| ATOM | 941 | C | GLY | 134 | 37.831 | 7.541 | 48.387 | 1.00 | 23.90 | B |
| ATOM | 942 | O | GLY | 134 | 37.399 | 8.030 | 47.344 | 1.00 | 25.12 | B |
| ATOM | 943 | N | ILE | 135 | 37.075 | 6.887 | 49.261 | 1.00 | 22.33 | B |
| ATOM | 944 | CA | ILE | 135 | 35.657 | 6.662 | 49.055 | 1.00 | 19.60 | B |
| ATOM | 945 | CB | ILE | 135 | 35.048 | 5.962 | 50.295 | 1.00 | 17.94 | B |
| ATOM | 946 | CG2 | ILE | 135 | 33.513 | 5.984 | 50.232 | 1.00 | 15.17 | B |
| ATOM | 947 | CG1 | ILE | 135 | 35.604 | 4.531 | 50.381 | 1.00 | 13.85 | B |
| ATOM | 948 | CD1 | ILE | 135 | 35.402 | 3.883 | 51.712 | 1.00 | 11.57 | B |
| ATOM | 949 | C | ILE | 135 | 34.886 | 7.941 | 48.751 | 1.00 | 19.64 | B |
| ATOM | 950 | O | ILE | 135 | 34.130 | 7.995 | 47.789 | 1.00 | 17.27 | B |
| ATOM | 951 | N | ILE | 136 | 35.090 | 8.971 | 49.566 | 1.00 | 19.64 | B |
| ATOM | 952 | CA | ILE | 136 | 34.383 | 10.229 | 49.377 | 1.00 | 19.00 | B |
| ATOM | 953 | CB | ILE | 136 | 34.758 | 11.219 | 50.486 | 1.00 | 18.34 | B |
| ATOM | 954 | CG2 | ILE | 136 | 34.174 | 12.595 | 50.188 | 1.00 | 19.49 | B |
| ATOM | 955 | CG1 | ILE | 136 | 34.226 | 10.669 | 51.838 | 1.00 | 18.91 | B |
| ATOM | 956 | CD1 | ILE | 136 | 34.680 | 11.447 | 53.086 | 1.00 | 18.92 | B |
| ATOM | 957 | C | ILE | 136 | 34.552 | 10.867 | 47.991 | 1.00 | 17.37 | B |
| ATOM | 958 | O | ILE | 136 | 33.614 | 10.888 | 47.207 | 1.00 | 15.94 | B |
| ATOM | 959 | N | PRO | 137 | 35.742 | 11.382 | 47.662 | 1.00 | 16.74 | B |
| ATOM | 960 | CD | PRO | 137 | 37.083 | 11.311 | 48.259 | 1.00 | 16.29 | B |
| ATOM | 961 | CA | PRO | 137 | 35.785 | 11.963 | 46.318 | 1.00 | 17.68 | B |
| ATOM | 962 | CB | PRO | 137 | 37.263 | 12.305 | 46.132 | 1.00 | 14.17 | B |
| ATOM | 963 | CG | PRO | 137 | 37.966 | 11.351 | 47.037 | 1.00 | 16.06 | B |
| ATOM | 964 | C | PRO | 137 | 35.229 | 11.025 | 45.232 | 1.00 | 20.66 | B |
| ATOM | 965 | O | PRO | 137 | 34.408 | 11.434 | 44.406 | 1.00 | 22.43 | B |
| ATOM | 966 | N | ARG | 138 | 35.651 | 9.764 | 45.232 | 1.00 | 21.33 | B |
| ATOM | 967 | CA | ARG | 138 | 35.154 | 8.825 | 44.224 | 1.00 | 21.16 | B |
| ATOM | 968 | CB | ARG | 138 | 35.768 | 7.428 | 44.436 | 1.00 | 19.87 | B |
| ATOM | 969 | CG | ARG | 138 | 37.251 | 7.370 | 44.138 | 1.00 | 18.07 | B |
| ATOM | 970 | CD | ARG | 138 | 37.812 | 5.989 | 44.402 | 1.00 | 17.00 | B |
| ATOM | 971 | NE | ARG | 138 | 39.264 | 6.019 | 44.408 | 1.00 | 14.48 | B |
| ATOM | 972 | CZ | ARG | 138 | 40.016 | 5.909 | 43.327 | 1.00 | 16.26 | B |
| ATOM | 973 | NH1 | ARG | 138 | 39.446 | 5.743 | 42.137 | 1.00 | 15.29 | B |
| ATOM | 974 | NH2 | ARG | 138 | 41.337 | 6.004 | 43.433 | 1.00 | 14.85 | B |
| ATOM | 975 | C | ARG | 138 | 33.630 | 8.705 | 44.202 | 1.00 | 21.32 | B |
| ATOM | 976 | O | ARG | 138 | 33.021 | 8.644 | 43.139 | 1.00 | 25.00 | B |
| ATOM | 977 | N | THR | 139 | 33.009 | 8.667 | 45.370 | 1.00 | 20.40 | B |
| ATOM | 978 | CA | THR | 139 | 31.562 | 8.540 | 45.436 | 1.00 | 20.86 | B |
| ATOM | 979 | CB | THR | 139 | 31.081 | 8.385 | 46.895 | 1.00 | 20.11 | B |
| ATOM | 980 | OG1 | THR | 139 | 31.770 | 7.293 | 47.512 | 1.00 | 21.18 | B |
| ATOM | 981 | CG2 | THR | 139 | 29.583 | 8.120 | 46.944 | 1.00 | 18.68 | B |
| ATOM | 982 | C | THR | 139 | 30.883 | 9.753 | 44.815 | 1.00 | 23.10 | B |
| ATOM | 983 | O | THR | 139 | 29.955 | 9.613 | 44.014 | 1.00 | 24.95 | B |
| ATOM | 984 | N | LEU | 140 | 31.340 | 10.944 | 45.189 | 1.00 | 23.71 | B |
| ATOM | 985 | CA | LEU | 140 | 30.762 | 12.175 | 44.659 | 1.00 | 23.38 | B |
| ATOM | 986 | CB | LEU | 140 | 31.480 | 13.401 | 45.238 | 1.00 | 21.47 | B |
| ATOM | 987 | CG | LEU | 140 | 31.211 | 13.560 | 46.733 | 1.00 | 21.91 | B |
| ATOM | 988 | CD1 | LEU | 140 | 32.120 | 14.621 | 47.305 | 1.00 | 21.37 | B |
| ATOM | 989 | CD2 | LEU | 140 | 29.740 | 13.883 | 46.966 | 1.00 | 18.69 | B |
| ATOM | 990 | C | LEU | 140 | 30.859 | 12.184 | 43.154 | 1.00 | 23.10 | B |
| ATOM | 991 | O | LEU | 140 | 29.870 | 12.395 | 42.467 | 1.00 | 21.86 | B |
| ATOM | 992 | N | HIS | 141 | 32.058 | 11.948 | 42.645 | 1.00 | 24.02 | B |
| ATOM | 993 | CA | HIS | 141 | 32.272 | 11.927 | 41.207 | 1.00 | 27.46 | B |
| ATOM | 994 | CB | HIS | 141 | 33.741 | 11.616 | 40.908 | 1.00 | 27.50 | B |
| ATOM | 995 | CG | HIS | 141 | 34.101 | 11.718 | 39.457 | 1.00 | 30.18 | B |
| ATOM | 996 | CD2 | HIS | 141 | 34.041 | 10.807 | 38.457 | 1.00 | 30.98 | B |
| ATOM | 997 | ND1 | HIS | 141 | 34.614 | 12.869 | 38.896 | 1.00 | 30.79 | B |
| ATOM | 998 | CE1 | HIS | 141 | 34.859 | 12.662 | 37.615 | 1.00 | 29.68 | B |
| ATOM | 999 | NE2 | HIS | 141 | 34.520 | 11.419 | 37.324 | 1.00 | 31.87 | B |
| ATOM | 1000 | C | HIS | 141 | 31.372 | 10.885 | 40.517 | 1.00 | 28.79 | B |
| ATOM | 1001 | O | HIS | 141 | 30.835 | 11.133 | 39.432 | 1.00 | 30.63 | B |
| ATOM | 1002 | N | GLN | 142 | 31.196 | 9.728 | 41.154 | 1.00 | 27.09 | B |
| ATOM | 1003 | CA | GLN | 142 | 30.392 | 8.664 | 40.579 | 1.00 | 26.11 | B |
| ATOM | 1004 | CB | GLN | 142 | 30.660 | 7.381 | 41.302 | 1.00 | 27.58 | B |
| ATOM | 1005 | CG | GLN | 142 | 31.938 | 6.733 | 40.855 | 1.00 | 29.72 | B |
| ATOM | 1006 | CD | GLN | 142 | 32.001 | 6.617 | 39.344 | 1.00 | 31.15 | B |
| ATOM | 1007 | OE1 | GLN | 142 | 31.181 | 5.929 | 38.729 | 1.00 | 32.85 | B |
| ATOM | 1008 | NE2 | GLN | 142 | 32.969 | 7.300 | 38.735 | 1.00 | 29.44 | B |
| ATOM | 1009 | C | GLN | 142 | 28.894 | 8.913 | 40.514 | 1.00 | 25.79 | B |
| ATOM | 1010 | O | GLN | 142 | 28.238 | 8.494 | 39.564 | 1.00 | 25.19 | B |
| ATOM | 1011 | N | ILE | 143 | 28.351 | 9.583 | 41.523 | 1.00 | 24.49 | B |
| ATOM | 1012 | CA | ILE | 143 | 26.928 | 9.888 | 41.555 | 1.00 | 23.07 | B |
| ATOM | 1013 | CB | ILE | 143 | 26.581 | 10.716 | 42.805 | 1.00 | 22.41 | B |
| ATOM | 1014 | CG2 | ILE | 143 | 25.174 | 11.285 | 42.690 | 1.00 | 24.89 | B |
| ATOM | 1015 | CG1 | ILE | 143 | 26.727 | 9.856 | 44.044 | 1.00 | 21.77 | B |
| ATOM | 1016 | CD1 | ILE | 143 | 26.477 | 10.599 | 45.339 | 1.00 | 21.34 | B |
| ATOM | 1017 | C | ILE | 143 | 26.492 | 10.664 | 40.308 | 1.00 | 23.84 | B |
| ATOM | 1018 | O | ILE | 143 | 25.417 | 10.425 | 39.769 | 1.00 | 23.49 | B |
| ATOM | 1019 | N | PHE | 144 | 27.334 | 11.593 | 39.860 | 1.00 | 25.75 | B |
| ATOM | 1020 | CA | PHE | 144 | 27.044 | 12.418 | 38.690 | 1.00 | 27.59 | B |
| ATOM | 1021 | CB | PHE | 144 | 28.019 | 13.657 | 38.638 | 1.00 | 26.93 | B |
| ATOM | 1022 | CG | PHE | 144 | 27.734 | 14.694 | 39.688 | 1.00 | 27.63 | B |
| ATOM | 1023 | CD1 | PHE | 144 | 26.583 | 15.478 | 39.614 | 1.00 | 28.58 | B |
| ATOM | 1024 | CD2 | PHE | 144 | 28.577 | 14.845 | 40.785 | 1.00 | 27.80 | B |
| ATOM | 1025 | CE1 | PHE | 144 | 26.271 | 16.396 | 40.626 | 1.00 | 28.69 | B |
| ATOM | 1026 | CE2 | PHE | 144 | 28.279 | 15.756 | 41.802 | 1.00 | 27.42 | B |
| ATOM | 1027 | CZ | PHE | 144 | 27.121 | 16.532 | 41.723 | 1.00 | 29.86 | B |
| ATOM | 1028 | C | PHE | 144 | 27.129 | 11.621 | 37.394 | 1.00 | 28.56 | B |
| ATOM | 1029 | O | PHE | 144 | 26.425 | 11.918 | 36.423 | 1.00 | 27.83 | B |
| ATOM | 1030 | N | GLU | 145 | 27.998 | 10.614 | 37.382 | 1.00 | 30.60 | B |
| ATOM | 1031 | CA | GLU | 145 | 28.160 | 9.757 | 36.209 | 1.00 | 32.75 | B |
| ATOM | 1032 | CB | GLU | 145 | 29.433 | 8.889 | 36.357 | 1.00 | 35.85 | B |
| ATOM | 1033 | CG | GLU | 145 | 30.742 | 9.673 | 36.317 | 1.00 | 42.03 | B |
| ATOM | 1034 | CD | GLU | 145 | 31.201 | 9.977 | 34.898 | 1.00 | 46.55 | B |
| ATOM | 1035 | OE1 | GLU | 145 | 32.014 | 10.916 | 34.699 | 1.00 | 47.36 | B |
| ATOM | 1036 | OE2 | GLU | 145 | 30.748 | 9.262 | 33.976 | 1.00 | 49.72 | B |
| ATOM | 1037 | C | GLU | 145 | 26.934 | 8.854 | 36.040 | 1.00 | 32.32 | B |
| ATOM | 1038 | O | GLU | 145 | 26.319 | 8.812 | 34.974 | 1.00 | 32.21 | B |
| ATOM | 1039 | N | LYS | 146 | 26.573 | 8.150 | 37.104 | 1.00 | 31.79 | B |
| ATOM | 1040 | CA | LYS | 146 | 25.443 | 7.235 | 37.066 | 1.00 | 34.10 | B |
| ATOM | 1041 | CB | LYS | 146 | 25.340 | 6.463 | 38.430 | 1.00 | 34.57 | B |
| ATOM | 1042 | CG | LYS | 146 | 26.693 | 5.973 | 38.952 | 1.00 | 35.68 | B |
| ATOM | 1043 | CD | LYS | 146 | 26.597 | 4.862 | 39.994 | 1.00 | 34.50 | B |
| ATOM | 1044 | CE | LYS | 146 | 26.566 | 3.486 | 39.327 | 1.00 | 35.54 | B |
| ATOM | 1045 | NZ | LYS | 146 | 27.115 | 2.405 | 40.204 | 1.00 | 33.09 | B |
| ATOM | 1046 | C | LYS | 146 | 24.098 | 7.888 | 36.721 | 1.00 | 34.95 | B |
| ATOM | 1047 | O | LYS | 146 | 23.320 | 7.342 | 35.929 | 1.00 | 35.60 | B |
| ATOM | 1048 | N | LEU | 147 | 23.831 | 9.057 | 37.298 | 1.00 | 34.40 | B |
| ATOM | 1049 | CA | LEU | 147 | 22.574 | 9.762 | 37.061 | 1.00 | 33.66 | B |
| ATOM | 1050 | CB | LEU | 147 | 22.154 | 10.477 | 38.336 | 1.00 | 32.95 | B |
| ATOM | 1051 | CG | LEU | 147 | 21.963 | 9.607 | 39.554 | 1.00 | 33.64 | B |
| ATOM | 1052 | CD1 | LEU | 147 | 21.682 | 10.474 | 40.775 | 1.00 | 34.40 | B |
| ATOM | 1053 | CD2 | LEU | 147 | 20.809 | 8.645 | 39.308 | 1.00 | 35.51 | B |
| ATOM | 1054 | C | LEU | 147 | 22.634 | 10.772 | 35.907 | 1.00 | 34.15 | B |
| ATOM | 1055 | O | LEU | 147 | 21.724 | 11.576 | 35.728 | 1.00 | 32.96 | B |
| ATOM | 1056 | N | THR | 148 | 23.698 | 10.719 | 35.115 | 1.00 | 35.64 | B |
| ATOM | 1057 | CA | THR | 148 | 23.863 | 11.656 | 34.011 | 1.00 | 36.46 | B |
| ATOM | 1058 | CB | THR | 148 | 25.138 | 11.332 | 33.198 | 1.00 | 35.78 | B |
| ATOM | 1059 | OG1 | THR | 148 | 25.492 | 12.468 | 32.409 | 1.00 | 36.67 | B |
| ATOM | 1060 | CG2 | THR | 148 | 24.914 | 10.150 | 32.274 | 1.00 | 36.63 | B |
| ATOM | 1061 | C | THR | 148 | 22.659 | 11.770 | 33.057 | 1.00 | 37.44 | B |
| ATOM | 1062 | O | THR | 148 | 22.313 | 12.878 | 32.639 | 1.00 | 37.93 | B |
| ATOM | 1063 | N | ASP | 149 | 22.019 | 10.653 | 32.712 | 1.00 | 35.78 | B |
| ATOM | 1064 | CA | ASP | 149 | 20.867 | 10.706 | 31.807 | 1.00 | 35.94 | B |
| ATOM | 1065 | CB | ASP | 149 | 21.337 | 11.004 | 30.322 | 1.00 | 34.77 | B |
| ATOM | 1066 | CG | ASP | 149 | 22.404 | 10.027 | 29.827 | 1.00 | 36.65 | B |
| ATOM | 1067 | OD1 | ASP | 149 | 22.605 | 8.965 | 30.467 | 1.00 | 35.17 | B |
| ATOM | 1068 | OD2 | ASP | 149 | 23.032 | 10.321 | 28.784 | 1.00 | 35.41 | B |
| ATOM | 1069 | C | ASP | 149 | 19.966 | 9.460 | 31.824 | 1.00 | 36.15 | B |
| ATOM | 1070 | O | ASP | 149 | 19.568 | 8.947 | 30.769 | 1.00 | 32.78 | B |
| ATOM | 1071 | N | ASN | 150 | 19.639 | 8.987 | 33.025 | 1.00 | 36.51 | B |
| ATOM | 1072 | CA | ASN | 150 | 18.781 | 7.819 | 33.181 | 1.00 | 38.16 | B |
| ATOM | 1073 | CB | ASN | 150 | 19.218 | 6.992 | 34.417 | 1.00 | 37.97 | B |
| ATOM | 1074 | CG | ASN | 150 | 19.159 | 7.785 | 35.704 | 1.00 | 37.13 | B |
| ATOM | 1075 | OD1 | ASN | 150 | 19.548 | 8.951 | 35.742 | 1.00 | 37.20 | B |
| ATOM | 1076 | ND2 | ASN | 150 | 18.694 | 7.148 | 36.774 | 1.00 | 36.82 | B |
| ATOM | 1077 | C | ASN | 150 | 17.314 | 8.240 | 33.305 | 1.00 | 39.47 | B |
| ATOM | 1078 | O | ASN | 150 | 16.419 | 7.397 | 33.433 | 1.00 | 39.49 | B |
| ATOM | 1079 | N | GLY | 151 | 17.077 | 9.549 | 33.245 | 1.00 | 39.29 | B |
| ATOM | 1080 | CA | GLY | 151 | 15.725 | 10.063 | 33.343 | 1.00 | 39.01 | B |
| ATOM | 1081 | C | GLY | 151 | 15.333 | 10.349 | 34.772 | 1.00 | 39.23 | B |
| ATOM | 1082 | O | GLY | 151 | 14.170 | 10.612 | 35.063 | 1.00 | 40.53 | B |
| ATOM | 1083 | N | THR | 152 | 16.307 | 10.285 | 35.670 | 1.00 | 40.25 | B |
| ATOM | 1084 | CA | THR | 152 | 16.069 | 10.547 | 37.085 | 1.00 | 40.87 | B |
| ATOM | 1085 | CB | THR | 152 | 16.730 | 9.463 | 37.960 | 1.00 | 39.78 | B |
| ATOM | 1086 | OG1 | THR | 152 | 16.146 | 8.191 | 37.655 | 1.00 | 43.27 | B |
| ATOM | 1087 | CG2 | THR | 152 | 16.531 | 9.764 | 39.437 | 1.00 | 40.09 | B |
| ATOM | 1088 | C | THR | 152 | 16.643 | 11.918 | 37.448 | 1.00 | 41.24 | B |
| ATOM | 1089 | O | THR | 152 | 17.860 | 12.120 | 37.434 | 1.00 | 42.84 | B |
| ATOM | 1090 | N | GLU | 153 | 15.753 | 12.856 | 37.754 | 1.00 | 40.50 | B |
| ATOM | 1091 | CA | GLU | 153 | 16.140 | 14.216 | 38.118 | 1.00 | 39.45 | B |
| ATOM | 1092 | CB | GLU | 153 | 14.910 | 15.143 | 38.054 | 1.00 | 41.77 | B |
| ATOM | 1093 | CG | GLU | 153 | 15.258 | 16.606 | 37.831 | 1.00 | 47.08 | B |
| ATOM | 1094 | CD | GLU | 153 | 15.903 | 16.847 | 36.474 | 1.00 | 49.24 | B |
| ATOM | 1095 | OE1 | GLU | 153 | 16.559 | 17.901 | 36.313 | 1.00 | 49.10 | B |
| ATOM | 1096 | OE2 | GLU | 153 | 15.747 | 15.988 | 35.570 | 1.00 | 49.10 | B |
| ATOM | 1097 | C | GLU | 153 | 16.697 | 14.170 | 39.538 | 1.00 | 36.82 | B |
| ATOM | 1098 | O | GLU | 153 | 16.140 | 13.472 | 40.387 | 1.00 | 35.59 | B |
| ATOM | 1099 | N | PHE | 154 | 17.770 | 14.919 | 39.807 | 1.00 | 33.77 | B |
| ATOM | 1100 | CA | PHE | 154 | 18.380 | 14.877 | 41.140 | 1.00 | 31.58 | B |
| ATOM | 1101 | CB | PHE | 154 | 19.302 | 13.644 | 41.212 | 1.00 | 29.10 | B |
| ATOM | 1102 | CG | PHE | 154 | 20.572 | 13.797 | 40.414 | 1.00 | 25.93 | B |
| ATOM | 1103 | CD1 | PHE | 154 | 21.763 | 14.165 | 41.038 | 1.00 | 25.72 | B |
| ATOM | 1104 | CD2 | PHE | 154 | 20.573 | 13.597 | 39.037 | 1.00 | 23.66 | B |
| ATOM | 1105 | CE1 | PHE | 154 | 22.941 | 14.328 | 40.297 | 1.00 | 26.03 | B |
| ATOM | 1106 | CE2 | PHE | 154 | 21.741 | 13.758 | 38.294 | 1.00 | 25.52 | B |
| ATOM | 1107 | CZ | PHE | 154 | 22.930 | 14.123 | 38.925 | 1.00 | 24.44 | B |
| ATOM | 1108 | C | PHE | 154 | 19.183 | 16.093 | 41.627 | 1.00 | 29.93 | B |
| ATOM | 1109 | O | PHE | 154 | 19.651 | 16.924 | 40.850 | 1.00 | 30.00 | B |
| ATOM | 1110 | N | SER | 155 | 19.357 | 16.157 | 42.940 | 1.00 | 28.97 | B |
| ATOM | 1111 | CA | SER | 155 | 20.140 | 17.212 | 43.572 | 1.00 | 28.90 | B |
| ATOM | 1112 | CB | SER | 155 | 19.225 | 18.281 | 44.243 | 1.00 | 26.53 | B |
| ATOM | 1113 | OG | SER | 155 | 18.732 | 17.844 | 45.502 | 1.00 | 24.48 | B |
| ATOM | 1114 | C | SER | 155 | 21.010 | 16.537 | 44.635 | 1.00 | 28.97 | B |
| ATOM | 1115 | O | SER | 155 | 20.588 | 15.569 | 45.279 | 1.00 | 28.86 | B |
| ATOM | 1116 | N | VAL | 156 | 22.221 | 17.047 | 44.819 | 1.00 | 29.35 | B |
| ATOM | 1117 | CA | VAL | 156 | 23.135 | 16.483 | 45.803 | 1.00 | 29.64 | B |
| ATOM | 1118 | CB | VAL | 156 | 24.431 | 15.977 | 45.125 | 1.00 | 28.79 | B |
| ATOM | 1119 | CG1 | VAL | 156 | 25.280 | 15.208 | 46.124 | 1.00 | 29.92 | B |
| ATOM | 1120 | CG2 | VAL | 156 | 24.089 | 15.116 | 43.930 | 1.00 | 29.12 | B |
| ATOM | 1121 | C | VAL | 156 | 23.516 | 17.517 | 46.863 | 1.00 | 29.76 | B |
| ATOM | 1122 | O | VAL | 156 | 23.925 | 18.627 | 46.532 | 1.00 | 30.11 | B |
| ATOM | 1123 | N | LYS | 157 | 23.372 | 17.149 | 48.132 | 1.00 | 30.23 | B |
| ATOM | 1124 | CA | LYS | 157 | 23.731 | 18.028 | 49.245 | 1.00 | 31.02 | B |
| ATOM | 1125 | CB | LYS | 157 | 22.489 | 18.431 | 50.063 | 1.00 | 32.19 | B |
| ATOM | 1126 | CG | LYS | 157 | 21.543 | 19.376 | 49.364 | 1.00 | 35.38 | B |
| ATOM | 1127 | CD | LYS | 157 | 20.246 | 19.523 | 50.162 | 1.00 | 39.38 | B |
| ATOM | 1128 | CE | LYS | 157 | 19.169 | 20.259 | 49.369 | 1.00 | 39.91 | B |
| ATOM | 1129 | NZ | LYS | 157 | 17.857 | 20.187 | 50.067 | 1.00 | 40.45 | B |
| ATOM | 1130 | C | LYS | 157 | 24.702 | 17.308 | 50.171 | 1.00 | 30.04 | B |
| ATOM | 1131 | O | LYS | 157 | 24.399 | 16.230 | 50.668 | 1.00 | 30.82 | B |
| ATOM | 1132 | N | VAL | 158 | 25.866 | 17.900 | 50.402 | 1.00 | 27.97 | B |
| ATOM | 1133 | CA | VAL | 158 | 26.839 | 17.290 | 51.292 | 1.00 | 27.63 | B |
| ATOM | 1134 | CB | VAL | 158 | 28.284 | 17.406 | 50.751 | 1.00 | 27.29 | B |
| ATOM | 1135 | CG1 | VAL | 158 | 28.433 | 16.582 | 49.478 | 1.00 | 29.26 | B |
| ATOM | 1136 | CG2 | VAL | 158 | 28.632 | 18.861 | 50.491 | 1.00 | 26.29 | B |
| ATOM | 1137 | C | VAL | 158 | 26.785 | 17.959 | 52.649 | 1.00 | 27.62 | B |
| ATOM | 1138 | O | VAL | 158 | 26.182 | 19.009 | 52.818 | 1.00 | 27.51 | B |
| ATOM | 1139 | N | SER | 159 | 27.431 | 17.344 | 53.624 | 1.00 | 28.77 | B |
| ATOM | 1140 | CA | SER | 159 | 27.449 | 17.896 | 54.962 | 1.00 | 29.25 | B |
| ATOM | 1141 | CB | SER | 159 | 26.155 | 17.634 | 55.612 | 1.00 | 29.36 | B |
| ATOM | 1142 | OG | SER | 159 | 26.083 | 18.324 | 56.835 | 1.00 | 35.64 | B |
| ATOM | 1143 | C | SER | 159 | 28.584 | 17.255 | 55.753 | 1.00 | 28.48 | B |
| ATOM | 1144 | O | SER | 159 | 28.762 | 16.037 | 55.723 | 1.00 | 29.46 | B |
| ATOM | 1145 | N | LEU | 160 | 29.364 | 18.070 | 56.451 | 1.00 | 26.66 | B |
| ATOM | 1146 | CA | LEU | 160 | 30.473 | 17.529 | 57.215 | 1.00 | 26.24 | B |
| ATOM | 1147 | CB | LEU | 160 | 31.769 | 18.008 | 56.649 | 1.00 | 26.22 | B |
| ATOM | 1148 | CG | LEU | 160 | 33.024 | 17.381 | 57.255 | 1.00 | 25.56 | B |
| ATOM | 1149 | CD1 | LEU | 160 | 32.850 | 15.873 | 57.350 | 1.00 | 24.56 | B |
| ATOM | 1150 | CD2 | LEU | 160 | 34.241 | 17.759 | 56.400 | 1.00 | 24.75 | B |
| ATOM | 1151 | C | LEU | 160 | 30.393 | 17.872 | 58.690 | 1.00 | 26.51 | B |
| ATOM | 1152 | O | LEU | 160 | 30.816 | 18.949 | 59.119 | 1.00 | 24.86 | B |
| ATOM | 1153 | N | LEU | 161 | 29.844 | 16.937 | 59.461 | 1.00 | 25.32 | B |
| ATOM | 1154 | CA | LEU | 161 | 29.686 | 17.112 | 60.895 | 1.00 | 23.81 | B |
| ATOM | 1155 | CB | LEU | 161 | 28.349 | 16.607 | 61.310 | 1.00 | 23.24 | B |
| ATOM | 1156 | CG | LEU | 161 | 28.109 | 16.490 | 62.766 | 1.00 | 23.19 | B |
| ATOM | 1157 | CD1 | LEU | 161 | 27.992 | 17.879 | 63.371 | 1.00 | 24.82 | B |
| ATOM | 1158 | CD2 | LEU | 161 | 26.838 | 15.701 | 62.989 | 1.00 | 22.84 | B |
| ATOM | 1159 | C | LEU | 161 | 30.777 | 16.338 | 61.613 | 1.00 | 24.19 | B |
| ATOM | 1160 | O | LEU | 161 | 31.024 | 15.178 | 61.307 | 1.00 | 25.43 | B |
| ATOM | 1161 | N | GLU | 162 | 31.444 | 16.983 | 62.563 | 1.00 | 23.56 | B |
| ATOM | 1162 | CA | GLU | 162 | 32.507 | 16.322 | 63.304 | 1.00 | 21.29 | B |
| ATOM | 1163 | CB | GLU | 162 | 33.892 | 16.895 | 62.872 | 1.00 | 19.65 | B |
| ATOM | 1164 | CG | GLU | 162 | 34.027 | 16.956 | 61.338 | 1.00 | 18.31 | B |
| ATOM | 1165 | CD | GLU | 162 | 35.463 | 16.923 | 60.845 | 1.00 | 19.90 | B |
| ATOM | 1166 | OE1 | GLU | 162 | 36.362 | 17.416 | 61.557 | 1.00 | 20.88 | B |
| ATOM | 1167 | OE2 | GLU | 162 | 35.699 | 16.413 | 59.729 | 1.00 | 21.08 | B |
| ATOM | 1168 | C | GLU | 162 | 32.276 | 16.448 | 64.803 | 1.00 | 21.51 | B |
| ATOM | 1169 | O | GLU | 162 | 31.734 | 17.441 | 65.286 | 1.00 | 24.11 | B |
| ATOM | 1170 | N | ILE | 163 | 32.665 | 15.419 | 65.543 | 1.00 | 20.50 | B |
| ATOM | 1171 | CA | ILE | 163 | 32.464 | 15.414 | 66.979 | 1.00 | 16.52 | B |
| ATOM | 1172 | CB | ILE | 163 | 31.587 | 14.221 | 67.396 | 1.00 | 15.68 | B |
| ATOM | 1173 | CG2 | ILE | 163 | 31.070 | 14.412 | 68.813 | 1.00 | 13.11 | B |
| ATOM | 1174 | CG1 | ILE | 163 | 30.420 | 14.093 | 66.427 | 1.00 | 14.88 | B |
| ATOM | 1175 | CD1 | ILE | 163 | 29.521 | 12.920 | 66.704 | 1.00 | 16.15 | B |
| ATOM | 1176 | C | ILE | 163 | 33.805 | 15.325 | 67.672 | 1.00 | 17.43 | B |
| ATOM | 1177 | O | ILE | 163 | 34.644 | 14.499 | 67.319 | 1.00 | 17.59 | B |
| ATOM | 1178 | N | TYR | 164 | 33.996 | 16.201 | 68.654 | 1.00 | 17.46 | B |
| ATOM | 1179 | CA | TYR | 164 | 35.219 | 16.263 | 69.430 | 1.00 | 16.57 | B |
| ATOM | 1180 | CB | TYR | 164 | 36.192 | 17.276 | 68.783 | 1.00 | 14.70 | B |
| ATOM | 1181 | CG | TYR | 164 | 37.464 | 17.474 | 69.559 | 1.00 | 12.25 | B |
| ATOM | 1182 | CD1 | TYR | 164 | 37.502 | 18.334 | 70.653 | 1.00 | 13.17 | B |
| ATOM | 1183 | CE1 | TYR | 164 | 38.643 | 18.439 | 71.454 | 1.00 | 15.94 | B |
| ATOM | 1184 | CD2 | TYR | 164 | 38.600 | 16.724 | 69.267 | 1.00 | 13.00 | B |
| ATOM | 1185 | CE2 | TYR | 164 | 39.753 | 16.814 | 70.058 | 1.00 | 15.22 | B |
| ATOM | 1186 | CZ | TYR | 164 | 39.773 | 17.674 | 71.155 | 1.00 | 17.31 | B |
| ATOM | 1187 | OH | TYR | 164 | 40.909 | 17.774 | 71.952 | 1.00 | 15.71 | B |
| ATOM | 1188 | C | TYR | 164 | 34.875 | 16.669 | 70.863 | 1.00 | 18.56 | B |
| ATOM | 1189 | O | TYR | 164 | 34.289 | 17.726 | 71.094 | 1.00 | 21.94 | B |
| ATOM | 1190 | N | ASN | 165 | 35.225 | 15.826 | 71.828 | 1.00 | 20.33 | B |
| ATOM | 1191 | CA | ASN | 165 | 34.942 | 16.122 | 73.232 | 1.00 | 22.94 | B |
| ATOM | 1192 | CB | ASN | 165 | 35.633 | 17.402 | 73.653 | 1.00 | 24.28 | B |
| ATOM | 1193 | CG | ASN | 165 | 36.418 | 17.255 | 74.942 | 1.00 | 28.53 | B |
| ATOM | 1194 | OD1 | ASN | 165 | 37.598 | 16.864 | 74.929 | 1.00 | 31.28 | B |
| ATOM | 1195 | ND2 | ASN | 165 | 35.777 | 17.569 | 76.064 | 1.00 | 24.86 | B |
| ATOM | 1196 | C | ASN | 165 | 33.443 | 16.314 | 73.406 | 1.00 | 24.90 | B |
| ATOM | 1197 | O | ASN | 165 | 33.009 | 17.222 | 74.121 | 1.00 | 26.77 | B |
| ATOM | 1198 | N | GLU | 166 | 32.657 | 15.471 | 72.745 | 1.00 | 23.40 | B |
| ATOM | 1199 | CA | GLU | 166 | 31.200 | 15.555 | 72.813 | 1.00 | 22.69 | B |
| ATOM | 1200 | CB | GLU | 166 | 30.706 | 15.231 | 74.237 | 1.00 | 22.07 | B |
| ATOM | 1201 | CG | GLU | 166 | 30.814 | 13.757 | 74.590 | 1.00 | 22.71 | B |
| ATOM | 1202 | CD | GLU | 166 | 30.157 | 12.849 | 73.548 | 1.00 | 23.19 | B |
| ATOM | 1203 | OE1 | GLU | 166 | 28.906 | 12.779 | 73.505 | 1.00 | 22.44 | B |
| ATOM | 1204 | OE2 | GLU | 166 | 30.899 | 12.211 | 72.769 | 1.00 | 21.71 | B |
| ATOM | 1205 | C | GLU | 166 | 30.610 | 16.884 | 72.349 | 1.00 | 22.21 | B |
| ATOM | 1206 | O | GLU | 166 | 29.491 | 17.228 | 72.709 | 1.00 | 22.53 | B |
| ATOM | 1207 | N | GLU | 167 | 31.363 | 17.631 | 71.545 | 1.00 | 24.18 | B |
| ATOM | 1208 | CA | GLU | 167 | 30.885 | 18.899 | 71.011 | 1.00 | 23.58 | B |
| ATOM | 1209 | CB | GLU | 167 | 31.825 | 20.009 | 71.365 | 1.00 | 28.43 | B |
| ATOM | 1210 | CG | GLU | 167 | 31.900 | 20.321 | 72.848 | 1.00 | 34.21 | B |
| ATOM | 1211 | CD | GLU | 167 | 32.857 | 21.470 | 73.142 | 1.00 | 40.07 | B |
| ATOM | 1212 | OE1 | GLU | 167 | 34.033 | 21.400 | 72.702 | 1.00 | 41.07 | B |
| ATOM | 1213 | OE2 | GLU | 167 | 32.431 | 22.441 | 73.812 | 1.00 | 43.47 | B |
| ATOM | 1214 | C | GLU | 167 | 30.800 | 18.766 | 69.500 | 1.00 | 22.74 | B |
| ATOM | 1215 | O | GLU | 167 | 31.659 | 18.142 | 68.884 | 1.00 | 23.08 | B |
| ATOM | 1216 | N | LEU | 168 | 29.766 | 19.347 | 68.904 | 1.00 | 21.20 | B |
| ATOM | 1217 | CA | LEU | 168 | 29.578 | 19.274 | 67.461 | 1.00 | 20.52 | B |
| ATOM | 1218 | CB | LEU | 168 | 28.088 | 19.156 | 67.125 | 1.00 | 21.09 | B |
| ATOM | 1219 | CG | LEU | 168 | 27.319 | 17.889 | 67.681 | 1.00 | 22.11 | B |
| ATOM | 1220 | CD1 | LEU | 168 | 28.249 | 16.663 | 67.622 | 1.00 | 15.69 | B |
| ATOM | 1221 | CD2 | LEU | 168 | 26.837 | 18.136 | 69.114 | 1.00 | 21.13 | B |
| ATOM | 1222 | C | LEU | 168 | 30.173 | 20.458 | 66.702 | 1.00 | 21.77 | B |
| ATOM | 1223 | O | LEU | 168 | 30.178 | 21.598 | 67.179 | 1.00 | 22.45 | B |
| ATOM | 1224 | N | PHE | 169 | 30.673 | 20.171 | 65.506 | 1.00 | 20.28 | B |
| ATOM | 1225 | CA | PHE | 169 | 31.282 | 21.180 | 64.665 | 1.00 | 19.17 | B |
| ATOM | 1226 | CB | PHE | 169 | 32.835 | 21.112 | 64.778 | 1.00 | 19.31 | B |
| ATOM | 1227 | CG | PHE | 169 | 33.345 | 21.308 | 66.177 | 1.00 | 19.18 | B |
| ATOM | 1228 | CD1 | PHE | 169 | 33.688 | 20.213 | 66.966 | 1.00 | 20.05 | B |
| ATOM | 1229 | CD2 | PHE | 169 | 33.434 | 22.591 | 66.722 | 1.00 | 18.70 | B |
| ATOM | 1230 | CE1 | PHE | 169 | 34.112 | 20.385 | 68.281 | 1.00 | 19.61 | B |
| ATOM | 1231 | CE2 | PHE | 169 | 33.852 | 22.782 | 68.027 | 1.00 | 18.44 | B |
| ATOM | 1232 | CZ | PHE | 169 | 34.193 | 21.676 | 68.814 | 1.00 | 22.70 | B |
| ATOM | 1233 | C | PHE | 169 | 30.865 | 20.981 | 63.220 | 1.00 | 20.25 | B |
| ATOM | 1234 | O | PHE | 169 | 30.476 | 19.880 | 62.808 | 1.00 | 20.20 | B |
| ATOM | 1235 | N | ASP | 170 | 30.949 | 22.064 | 62.462 | 1.00 | 19.31 | B |
| ATOM | 1236 | CA | ASP | 170 | 30.603 | 22.069 | 61.053 | 1.00 | 19.06 | B |
| ATOM | 1237 | CB | ASP | 170 | 29.549 | 23.141 | 60.785 | 1.00 | 19.49 | B |
| ATOM | 1238 | CG | ASP | 170 | 28.970 | 23.066 | 59.386 | 1.00 | 21.37 | B |
| ATOM | 1239 | OD1 | ASP | 170 | 29.648 | 22.556 | 58.463 | 1.00 | 20.46 | B |
| ATOM | 1240 | OD2 | ASP | 170 | 27.827 | 23.542 | 59.206 | 1.00 | 24.10 | B |
| ATOM | 1241 | C | ASP | 170 | 31.902 | 22.429 | 60.353 | 1.00 | 20.21 | B |
| ATOM | 1242 | O | ASP | 170 | 32.402 | 23.540 | 60.509 | 1.00 | 21.52 | B |
| ATOM | 1243 | N | LEU | 171 | 32.460 | 21.492 | 59.599 | 1.00 | 20.15 | B |
| ATOM | 1244 | CA | LEU | 171 | 33.699 | 21.758 | 58.900 | 1.00 | 22.53 | B |
| ATOM | 1245 | CB | LEU | 171 | 34.620 | 20.517 | 58.965 | 1.00 | 19.76 | B |
| ATOM | 1246 | CG | LEU | 171 | 35.385 | 20.297 | 60.340 | 1.00 | 18.93 | B |
| ATOM | 1247 | CD1 | LEU | 171 | 36.562 | 21.251 | 60.487 | 1.00 | 16.80 | B |
| ATOM | 1248 | CD2 | LEU | 171 | 34.426 | 20.479 | 61.495 | 1.00 | 18.41 | B |
| ATOM | 1249 | C | LEU | 171 | 33.460 | 22.198 | 57.459 | 1.00 | 24.95 | B |
| ATOM | 1250 | O | LEU | 171 | 34.374 | 22.169 | 56.632 | 1.00 | 25.06 | B |
| ATOM | 1251 | N | LEU | 172 | 32.233 | 22.618 | 57.160 | 1.00 | 28.25 | B |
| ATOM | 1252 | CA | LEU | 172 | 31.910 | 23.081 | 55.812 | 1.00 | 33.55 | B |
| ATOM | 1253 | CB | LEU | 172 | 31.001 | 22.111 | 55.116 | 1.00 | 33.77 | B |
| ATOM | 1254 | CG | LEU | 172 | 31.664 | 20.867 | 54.556 | 1.00 | 34.20 | B |
| ATOM | 1255 | CD1 | LEU | 172 | 30.632 | 20.056 | 53.783 | 1.00 | 33.48 | B |
| ATOM | 1256 | CD2 | LEU | 172 | 32.807 | 21.268 | 53.644 | 1.00 | 34.44 | B |
| ATOM | 1257 | C | LEU | 172 | 31.279 | 24.461 | 55.766 | 1.00 | 35.97 | B |
| ATOM | 1258 | O | LEU | 172 | 31.181 | 25.059 | 54.706 | 1.00 | 37.85 | B |
| ATOM | 1259 | N | ASN | 173 | 30.843 | 24.962 | 56.912 | 1.00 | 39.07 | B |
| ATOM | 1260 | CA | ASN | 173 | 30.242 | 26.284 | 56.972 | 1.00 | 44.33 | B |
| ATOM | 1261 | CB | ASN | 173 | 29.451 | 26.445 | 58.275 | 1.00 | 45.10 | B |
| ATOM | 1262 | CG | ASN | 173 | 28.700 | 27.765 | 58.345 | 1.00 | 47.21 | B |
| ATOM | 1263 | OD1 | ASN | 173 | 27.898 | 27.987 | 59.254 | 1.00 | 46.55 | B |
| ATOM | 1264 | ND2 | ASN | 173 | 28.958 | 28.650 | 57.384 | 1.00 | 47.66 | B |
| ATOM | 1265 | C | ASN | 173 | 31.355 | 27.330 | 56.903 | 1.00 | 48.18 | B |
| ATOM | 1266 | O | ASN | 173 | 32.094 | 27.532 | 57.871 | 1.00 | 47.58 | B |
| ATOM | 1267 | N | PRO | 174 | 31.492 | 28.007 | 55.752 | 1.00 | 51.96 | B |
| ATOM | 1268 | CD | PRO | 174 | 30.737 | 27.802 | 54.502 | 1.00 | 52.92 | B |
| ATOM | 1269 | CA | PRO | 174 | 32.527 | 29.030 | 55.572 | 1.00 | 55.50 | B |
| ATOM | 1270 | CB | PRO | 174 | 32.609 | 29.162 | 54.076 | 1.00 | 54.73 | B |
| ATOM | 1271 | CG | PRO | 174 | 31.184 | 28.973 | 53.660 | 1.00 | 53.60 | B |
| ATOM | 1272 | C | PRO | 174 | 32.226 | 30.364 | 56.259 | 1.00 | 58.47 | B |
| ATOM | 1273 | O | PRO | 174 | 33.076 | 31.256 | 56.286 | 1.00 | 59.03 | B |
| ATOM | 1274 | N | SER | 175 | 31.024 | 30.497 | 56.819 | 1.00 | 60.76 | B |
| ATOM | 1275 | CA | SER | 175 | 30.639 | 31.730 | 57.504 | 1.00 | 62.73 | B |
| ATOM | 1276 | CB | SER | 175 | 29.138 | 32.013 | 57.301 | 1.00 | 63.76 | B |
| ATOM | 1277 | OG | SER | 175 | 28.877 | 32.450 | 55.975 | 1.00 | 66.00 | B |
| ATOM | 1278 | C | SER | 175 | 30.957 | 31.725 | 59.000 | 1.00 | 63.50 | B |
| ATOM | 1279 | O | SER | 175 | 30.901 | 32.769 | 59.654 | 1.00 | 63.94 | B |
| ATOM | 1280 | N | SER | 176 | 31.293 | 30.557 | 59.543 | 1.00 | 63.63 | B |
| ATOM | 1281 | CA | SER | 176 | 31.613 | 30.456 | 60.964 | 1.00 | 63.17 | B |
| ATOM | 1282 | CB | SER | 176 | 30.589 | 29.549 | 61.694 | 1.00 | 63.04 | B |
| ATOM | 1283 | OG | SER | 176 | 30.805 | 28.181 | 61.389 | 1.00 | 64.15 | B |
| ATOM | 1284 | C | SER | 176 | 33.017 | 29.909 | 61.188 | 1.00 | 62.90 | B |
| ATOM | 1285 | O | SER | 176 | 33.758 | 29.643 | 60.238 | 1.00 | 62.07 | B |
| ATOM | 1286 | N | ASP | 177 | 33.371 | 29.744 | 62.459 | 1.00 | 62.85 | B |
| ATOM | 1287 | CA | ASP | 177 | 34.676 | 29.225 | 62.837 | 1.00 | 62.62 | B |
| ATOM | 1288 | CB | ASP | 177 | 35.352 | 30.147 | 63.856 | 1.00 | 63.20 | B |
| ATOM | 1289 | CG | ASP | 177 | 35.504 | 31.559 | 63.345 | 1.00 | 63.21 | B |
| ATOM | 1290 | OD1 | ASP | 177 | 36.062 | 31.729 | 62.243 | 1.00 | 63.09 | B |
| ATOM | 1291 | OD2 | ASP | 177 | 35.068 | 32.498 | 64.044 | 1.00 | 62.91 | B |
| ATOM | 1292 | C | ASP | 177 | 34.515 | 27.852 | 63.452 | 1.00 | 61.87 | B |
| ATOM | 1293 | O | ASP | 177 | 33.447 | 27.504 | 63.954 | 1.00 | 62.79 | B |
| ATOM | 1294 | N | VAL | 178 | 35.588 | 27.078 | 63.415 | 1.00 | 60.45 | B |
| ATOM | 1295 | CA | VAL | 178 | 35.572 | 25.743 | 63.977 | 1.00 | 59.51 | B |
| ATOM | 1296 | CB | VAL | 178 | 36.894 | 25.005 | 63.688 | 1.00 | 59.52 | B |
| ATOM | 1297 | CG1 | VAL | 178 | 37.118 | 24.909 | 62.183 | 1.00 | 59.92 | B |
| ATOM | 1298 | CG2 | VAL | 178 | 38.048 | 25.729 | 64.356 | 1.00 | 59.97 | B |
| ATOM | 1299 | C | VAL | 178 | 35.363 | 25.834 | 65.485 | 1.00 | 58.12 | B |
| ATOM | 1300 | O | VAL | 178 | 35.159 | 24.825 | 66.157 | 1.00 | 59.80 | B |
| ATOM | 1301 | N | SER | 179 | 35.421 | 27.047 | 66.016 | 1.00 | 55.31 | B |
| ATOM | 1302 | CA | SER | 179 | 35.221 | 27.245 | 67.443 | 1.00 | 52.98 | B |
| ATOM | 1303 | CB | SER | 179 | 35.823 | 28.578 | 67.871 | 1.00 | 51.75 | B |
| ATOM | 1304 | OG | SER | 179 | 35.401 | 29.619 | 67.011 | 1.00 | 50.71 | B |
| ATOM | 1305 | C | SER | 179 | 33.725 | 27.211 | 67.746 | 1.00 | 52.04 | B |
| ATOM | 1306 | O | SER | 179 | 33.313 | 26.894 | 68.860 | 1.00 | 52.07 | B |
| ATOM | 1307 | N | GLU | 180 | 32.917 | 27.535 | 66.743 | 1.00 | 51.08 | B |
| ATOM | 1308 | CA | GLU | 180 | 31.467 | 27.541 | 66.882 | 1.00 | 50.67 | B |
| ATOM | 1309 | CB | GLU | 180 | 30.834 | 28.188 | 65.639 | 1.00 | 53.74 | B |
| ATOM | 1310 | CG | GLU | 180 | 29.322 | 28.334 | 65.691 | 1.00 | 57.88 | B |
| ATOM | 1311 | CD | GLU | 180 | 28.872 | 29.401 | 66.666 | 1.00 | 60.00 | B |
| ATOM | 1312 | OE1 | GLU | 180 | 29.192 | 29.279 | 67.868 | 1.00 | 61.89 | B |
| ATOM | 1313 | OE2 | GLU | 180 | 28.199 | 30.362 | 66.230 | 1.00 | 61.08 | B |
| ATOM | 1314 | C | GLU | 180 | 30.989 | 26.096 | 67.026 | 1.00 | 48.91 | B |
| ATOM | 1315 | O | GLU | 180 | 31.307 | 25.249 | 66.196 | 1.00 | 49.20 | B |
| ATOM | 1316 | N | ARG | 181 | 30.234 | 25.817 | 68.082 | 1.00 | 46.31 | B |
| ATOM | 1317 | CA | ARG | 181 | 29.739 | 24.472 | 68.332 | 1.00 | 44.31 | B |
| ATOM | 1318 | CB | ARG | 181 | 30.194 | 24.018 | 69.710 | 1.00 | 46.69 | B |
| ATOM | 1319 | CG | ARG | 181 | 29.815 | 24.962 | 70.842 | 1.00 | 50.74 | B |
| ATOM | 1320 | CD | ARG | 181 | 28.527 | 24.530 | 71.547 | 1.00 | 55.78 | B |
| ATOM | 1321 | NE | ARG | 181 | 28.677 | 23.242 | 72.234 | 1.00 | 60.23 | B |
| ATOM | 1322 | CZ | ARG | 181 | 27.708 | 22.628 | 72.913 | 1.00 | 61.32 | B |
| ATOM | 1323 | NH1 | ARG | 181 | 26.501 | 23.180 | 73.007 | 1.00 | 61.66 | B |
| ATOM | 1324 | NH2 | ARG | 181 | 27.945 | 21.453 | 73.490 | 1.00 | 61.67 | B |
| ATOM | 1325 | C | ARG | 181 | 28.217 | 24.395 | 68.211 | 1.00 | 42.65 | B |
| ATOM | 1326 | O | ARG | 181 | 27.491 | 25.115 | 68.888 | 1.00 | 42.59 | B |
| ATOM | 1327 | N | LEU | 182 | 27.739 | 23.510 | 67.344 | 1.00 | 39.35 | B |
| ATOM | 1328 | CA | LEU | 182 | 26.310 | 23.355 | 67.110 | 1.00 | 35.22 | B |
| ATOM | 1329 | CB | LEU | 182 | 26.088 | 22.559 | 65.843 | 1.00 | 32.83 | B |
| ATOM | 1330 | CG | LEU | 182 | 26.998 | 22.979 | 64.710 | 1.00 | 31.23 | B |
| ATOM | 1331 | CD1 | LEU | 182 | 26.730 | 22.114 | 63.508 | 1.00 | 32.55 | B |
| ATOM | 1332 | CD2 | LEU | 182 | 26.776 | 24.444 | 64.386 | 1.00 | 31.45 | B |
| ATOM | 1333 | C | LEU | 182 | 25.581 | 22.690 | 68.260 | 1.00 | 33.98 | B |
| ATOM | 1334 | O | LEU | 182 | 26.197 | 22.057 | 69.117 | 1.00 | 33.33 | B |
| ATOM | 1335 | N | GLN | 183 | 24.259 | 22.843 | 68.266 | 1.00 | 33.26 | B |
| ATOM | 1336 | CA | GLN | 183 | 23.399 | 22.259 | 69.296 | 1.00 | 32.84 | B |
| ATOM | 1337 | CB | GLN | 183 | 22.430 | 23.320 | 69.842 | 1.00 | 34.22 | B |
| ATOM | 1338 | CG | GLN | 183 | 23.122 | 24.542 | 70.436 | 1.00 | 37.39 | B |
| ATOM | 1339 | CD | GLN | 183 | 22.163 | 25.699 | 70.671 | 1.00 | 38.77 | B |
| ATOM | 1340 | OE1 | GLN | 183 | 21.325 | 26.003 | 69.818 | 1.00 | 39.62 | B |
| ATOM | 1341 | NE2 | GLN | 183 | 22.294 | 26.361 | 71.820 | 1.00 | 37.72 | B |
| ATOM | 1342 | C | GLN | 183 | 22.603 | 21.099 | 68.706 | 1.00 | 31.57 | B |
| ATOM | 1343 | O | GLN | 183 | 22.209 | 21.134 | 67.545 | 1.00 | 31.18 | B |
| ATOM | 1344 | N | MET | 184 | 22.353 | 20.079 | 69.513 | 1.00 | 31.59 | B |
| ATOM | 1345 | CA | MET | 184 | 21.622 | 18.908 | 69.052 | 1.00 | 32.44 | B |
| ATOM | 1346 | CB | MET | 184 | 22.480 | 17.677 | 69.297 | 1.00 | 32.63 | B |
| ATOM | 1347 | CG | MET | 184 | 22.018 | 16.404 | 68.626 | 1.00 | 34.09 | B |
| ATOM | 1348 | SD | MET | 184 | 23.162 | 15.016 | 68.908 | 1.00 | 32.00 | B |
| ATOM | 1349 | CE | MET | 184 | 22.574 | 14.436 | 70.488 | 1.00 | 31.68 | B |
| ATOM | 1350 | C | MET | 184 | 20.289 | 18.787 | 69.791 | 1.00 | 34.68 | B |
| ATOM | 1351 | O | MET | 184 | 20.203 | 19.114 | 70.976 | 1.00 | 35.18 | B |
| ATOM | 1352 | N | PHE | 185 | 19.248 | 18.345 | 69.086 | 1.00 | 36.66 | B |
| ATOM | 1353 | CA | PHE | 185 | 17.922 | 18.168 | 69.690 | 1.00 | 39.01 | B |
| ATOM | 1354 | CB | PHE | 185 | 16.987 | 19.422 | 69.462 | 1.00 | 37.84 | B |
| ATOM | 1355 | CG | PHE | 185 | 17.676 | 20.750 | 69.619 | 1.00 | 38.18 | B |
| ATOM | 1356 | CD1 | PHE | 185 | 18.453 | 21.270 | 68.593 | 1.00 | 36.50 | B |
| ATOM | 1357 | CD2 | PHE | 185 | 17.534 | 21.488 | 70.793 | 1.00 | 38.31 | B |
| ATOM | 1358 | CE1 | PHE | 185 | 19.080 | 22.502 | 68.724 | 1.00 | 36.83 | B |
| ATOM | 1359 | CE2 | PHE | 185 | 18.158 | 22.724 | 70.936 | 1.00 | 38.32 | B |
| ATOM | 1360 | CZ | PHE | 185 | 18.933 | 23.232 | 69.897 | 1.00 | 38.06 | B |
| ATOM | 1361 | C | PHE | 185 | 17.224 | 16.956 | 69.077 | 1.00 | 40.70 | B |
| ATOM | 1362 | O | PHE | 185 | 17.485 | 16.598 | 67.931 | 1.00 | 39.58 | B |
| ATOM | 1363 | N | ASP | 186 | 16.333 | 16.330 | 69.838 | 1.00 | 43.77 | B |
| ATOM | 1364 | CA | ASP | 186 | 15.588 | 15.187 | 69.328 | 1.00 | 46.67 | B |
| ATOM | 1365 | CB | ASP | 186 | 14.737 | 14.550 | 70.419 | 1.00 | 47.89 | B |
| ATOM | 1366 | CG | ASP | 186 | 15.534 | 14.206 | 71.659 | 1.00 | 50.45 | B |
| ATOM | 1367 | OD1 | ASP | 186 | 16.535 | 13.461 | 71.540 | 1.00 | 50.63 | B |
| ATOM | 1368 | OD2 | ASP | 186 | 15.154 | 14.679 | 72.756 | 1.00 | 51.23 | B |
| ATOM | 1369 | C | ASP | 186 | 14.668 | 15.740 | 68.262 | 1.00 | 47.79 | B |
| ATOM | 1370 | O | ASP | 186 | 14.371 | 16.933 | 68.246 | 1.00 | 47.04 | B |
| ATOM | 1371 | N | ASP | 187 | 14.215 | 14.883 | 67.365 | 1.00 | 50.77 | B |
| ATOM | 1372 | CA | ASP | 187 | 13.318 | 15.351 | 66.328 | 1.00 | 54.90 | B |
| ATOM | 1373 | CB | ASP | 187 | 13.748 | 14.832 | 64.990 | 1.00 | 56.93 | B |
| ATOM | 1374 | CG | ASP | 187 | 12.973 | 15.457 | 63.860 | 1.00 | 59.28 | B |
| ATOM | 1375 | OD1 | ASP | 187 | 13.425 | 15.343 | 62.700 | 1.00 | 60.01 | B |
| ATOM | 1376 | OD2 | ASP | 187 | 11.910 | 16.060 | 64.138 | 1.00 | 60.38 | B |
| ATOM | 1377 | C | ASP | 187 | 11.915 | 14.877 | 66.662 | 1.00 | 56.34 | B |
| ATOM | 1378 | O | ASP | 187 | 11.638 | 13.678 | 66.649 | 1.00 | 56.08 | B |
| ATOM | 1379 | N | PRO | 188 | 11.015 | 15.820 | 66.985 | 1.00 | 58.11 | B |
| ATOM | 1380 | CD | PRO | 188 | 11.251 | 17.274 | 66.963 | 1.00 | 57.99 | B |
| ATOM | 1381 | CA | PRO | 188 | 9.621 | 15.529 | 67.339 | 1.00 | 60.11 | B |
| ATOM | 1382 | CB | PRO | 188 | 8.978 | 16.890 | 67.309 | 1.00 | 59.76 | B |
| ATOM | 1383 | CG | PRO | 188 | 10.091 | 17.790 | 67.764 | 1.00 | 58.23 | B |
| ATOM | 1384 | C | PRO | 188 | 8.956 | 14.549 | 66.376 | 1.00 | 61.87 | B |
| ATOM | 1385 | O | PRO | 188 | 8.162 | 13.700 | 66.783 | 1.00 | 61.46 | B |
| ATOM | 1386 | N | ARG | 189 | 9.302 | 14.669 | 65.100 | 1.00 | 64.31 | B |
| ATOM | 1387 | CA | ARG | 189 | 8.757 | 13.812 | 64.058 | 1.00 | 66.68 | B |
| ATOM | 1388 | CB | ARG | 189 | 9.307 | 14.265 | 62.701 | 1.00 | 66.61 | B |
| ATOM | 1389 | CG | ARG | 189 | 8.813 | 15.651 | 62.277 | 1.00 | 66.58 | B |
| ATOM | 1390 | CD | ARG | 189 | 9.586 | 16.213 | 61.080 | 1.00 | 66.65 | B |
| ATOM | 1391 | NE | ARG | 189 | 10.834 | 16.866 | 61.474 | 1.00 | 66.32 | B |
| ATOM | 1392 | CZ | ARG | 189 | 11.704 | 17.407 | 60.625 | 1.00 | 66.09 | B |
| ATOM | 1393 | NH1 | ARG | 189 | 11.474 | 17.377 | 59.319 | 1.00 | 66.33 | B |
| ATOM | 1394 | NH2 | ARG | 189 | 12.803 | 17.988 | 61.083 | 1.00 | 65.55 | B |
| ATOM | 1395 | C | ARG | 189 | 9.041 | 12.321 | 64.289 | 1.00 | 68.64 | B |
| ATOM | 1396 | O | ARG | 189 | 8.300 | 11.461 | 63.813 | 1.00 | 69.00 | B |
| ATOM | 1397 | N | ASN | 190 | 10.110 | 12.018 | 65.022 | 1.00 | 71.07 | B |
| ATOM | 1398 | CA | ASN | 190 | 10.487 | 10.634 | 65.329 | 1.00 | 72.28 | B |
| ATOM | 1399 | CB | ASN | 190 | 10.758 | 9.814 | 63.998 | 1.00 | 72.30 | B |
| ATOM | 1400 | CG | ASN | 190 | 11.706 | 10.525 | 63.041 | 1.00 | 71.90 | B |
| ATOM | 1401 | OD1 | ASN | 190 | 12.847 | 10.822 | 63.385 | 1.00 | 71.47 | B |
| ATOM | 1402 | ND2 | ASN | 190 | 11.233 | 10.789 | 61.826 | 1.00 | 71.27 | B |
| ATOM | 1403 | C | ASN | 190 | 11.709 | 10.579 | 66.252 | 1.00 | 73.09 | B |
| ATOM | 1404 | O | ASN | 190 | 12.783 | 11.067 | 65.905 | 1.00 | 73.71 | B |
| ATOM | 1405 | N | LYS | 191 | 11.534 | 9.979 | 67.427 | 1.00 | 73.58 | B |
| ATOM | 1406 | CA | LYS | 191 | 12.601 | 9.871 | 68.428 | 1.00 | 73.23 | B |
| ATOM | 1407 | CB | LYS | 191 | 12.123 | 9.021 | 69.606 | 1.00 | 75.05 | B |
| ATOM | 1408 | CG | LYS | 191 | 11.285 | 9.778 | 70.614 | 1.00 | 76.84 | B |
| ATOM | 1409 | CD | LYS | 191 | 12.074 | 10.920 | 71.241 | 1.00 | 77.87 | B |
| ATOM | 1410 | CE | LYS | 191 | 11.299 | 11.547 | 72.387 | 1.00 | 78.94 | B |
| ATOM | 1411 | NZ | LYS | 191 | 9.939 | 11.988 | 71.961 | 1.00 | 79.06 | B |
| ATOM | 1412 | C | LYS | 191 | 13.965 | 9.351 | 67.968 | 1.00 | 71.65 | B |
| ATOM | 1413 | O | LYS | 191 | 15.000 | 9.869 | 68.395 | 1.00 | 71.97 | B |
| ATOM | 1414 | N | ARG | 192 | 13.977 | 8.326 | 67.121 | 1.00 | 68.70 | B |
| ATOM | 1415 | CA | ARG | 192 | 15.238 | 7.772 | 66.638 | 1.00 | 65.72 | B |
| ATOM | 1416 | CB | ARG | 192 | 14.978 | 6.515 | 65.768 | 1.00 | 67.67 | B |
| ATOM | 1417 | CG | ARG | 192 | 16.217 | 5.978 | 65.052 | 1.00 | 69.51 | B |
| ATOM | 1418 | CD | ARG | 192 | 16.068 | 4.519 | 64.616 | 1.00 | 70.83 | B |
| ATOM | 1419 | NE | ARG | 192 | 14.855 | 4.261 | 63.839 | 1.00 | 71.87 | B |
| ATOM | 1420 | CZ | ARG | 192 | 13.672 | 3.950 | 64.364 | 1.00 | 71.73 | B |
| ATOM | 1421 | NH1 | ARG | 192 | 13.527 | 3.855 | 65.681 | 1.00 | 70.61 | B |
| ATOM | 1422 | NH2 | ARG | 192 | 12.631 | 3.727 | 63.569 | 1.00 | 71.53 | B |
| ATOM | 1423 | C | ARG | 192 | 16.033 | 8.803 | 65.843 | 1.00 | 62.08 | B |
| ATOM | 1424 | O | ARG | 192 | 17.190 | 8.572 | 65.482 | 1.00 | 61.32 | B |
| ATOM | 1425 | N | GLY | 193 | 15.403 | 9.946 | 65.585 | 1.00 | 58.42 | B |
| ATOM | 1426 | CA | GLY | 193 | 16.045 | 11.008 | 64.828 | 1.00 | 52.07 | B |
| ATOM | 1427 | C | GLY | 193 | 16.519 | 12.171 | 65.674 | 1.00 | 47.14 | B |
| ATOM | 1428 | O | GLY | 193 | 16.159 | 12.300 | 66.843 | 1.00 | 46.94 | B |
| ATOM | 1429 | N | VAL | 194 | 17.323 | 13.033 | 65.067 | 1.00 | 44.16 | B |
| ATOM | 1430 | CA | VAL | 194 | 17.875 | 14.184 | 65.757 | 1.00 | 40.67 | B |
| ATOM | 1431 | CB | VAL | 194 | 19.266 | 13.838 | 66.329 | 1.00 | 39.96 | B |
| ATOM | 1432 | CG1 | VAL | 194 | 20.338 | 14.058 | 65.271 | 1.00 | 37.96 | B |
| ATOM | 1433 | CG2 | VAL | 194 | 19.539 | 14.653 | 67.564 | 1.00 | 39.63 | B |
| ATOM | 1434 | C | VAL | 194 | 18.008 | 15.373 | 64.800 | 1.00 | 39.90 | B |
| ATOM | 1435 | O | VAL | 194 | 18.145 | 15.194 | 63.592 | 1.00 | 40.91 | B |
| ATOM | 1436 | N | ILE | 195 | 17.965 | 16.585 | 65.347 | 1.00 | 38.55 | B |
| ATOM | 1437 | CA | ILE | 195 | 18.104 | 17.803 | 64.553 | 1.00 | 35.81 | B |
| ATOM | 1438 | CB | ILE | 195 | 16.862 | 18.728 | 64.709 | 1.00 | 38.25 | B |
| ATOM | 1439 | CG2 | ILE | 195 | 17.132 | 20.092 | 64.055 | 1.00 | 38.19 | B |
| ATOM | 1440 | CG1 | ILE | 195 | 15.615 | 18.049 | 64.084 | 1.00 | 39.77 | B |
| ATOM | 1441 | CD1 | ILE | 195 | 14.321 | 18.863 | 64.185 | 1.00 | 41.59 | B |
| ATOM | 1442 | C | ILE | 195 | 19.347 | 18.581 | 65.001 | 1.00 | 32.57 | B |
| ATOM | 1443 | O | ILE | 195 | 19.452 | 18.970 | 66.162 | 1.00 | 30.74 | B |
| ATOM | 1444 | N | ILE | 196 | 20.292 | 18.787 | 64.086 | 1.00 | 29.82 | B |
| ATOM | 1445 | CA | ILE | 196 | 21.500 | 19.539 | 64.405 | 1.00 | 27.94 | B |
| ATOM | 1446 | CB | ILE | 196 | 22.800 | 18.919 | 63.769 | 1.00 | 26.64 | B |
| ATOM | 1447 | CG2 | ILE | 196 | 24.006 | 19.816 | 64.070 | 1.00 | 21.22 | B |
| ATOM | 1448 | CG1 | ILE | 196 | 23.110 | 17.510 | 64.383 | 1.00 | 24.18 | B |
| ATOM | 1449 | CD1 | ILE | 196 | 22.375 | 16.374 | 63.764 | 1.00 | 22.10 | B |
| ATOM | 1450 | C | ILE | 196 | 21.303 | 20.951 | 63.872 | 1.00 | 27.99 | B |
| ATOM | 1451 | O | ILE | 196 | 21.375 | 21.196 | 62.669 | 1.00 | 27.68 | B |
| ATOM | 1452 | N | LYS | 197 | 21.044 | 21.876 | 64.784 | 1.00 | 29.44 | B |
| ATOM | 1453 | CA | LYS | 197 | 20.813 | 23.265 | 64.426 | 1.00 | 30.91 | B |
| ATOM | 1454 | CB | LYS | 197 | 20.205 | 24.026 | 65.616 | 1.00 | 33.42 | B |
| ATOM | 1455 | CG | LYS | 197 | 19.931 | 25.486 | 65.303 | 1.00 | 35.76 | B |
| ATOM | 1456 | CD | LYS | 197 | 19.670 | 26.299 | 66.548 | 1.00 | 39.21 | B |
| ATOM | 1457 | CE | LYS | 197 | 19.686 | 27.776 | 66.199 | 1.00 | 42.14 | B |
| ATOM | 1458 | NZ | LYS | 197 | 20.909 | 28.121 | 65.411 | 1.00 | 42.07 | B |
| ATOM | 1459 | C | LYS | 197 | 22.073 | 23.984 | 63.971 | 1.00 | 29.67 | B |
| ATOM | 1460 | O | LYS | 197 | 23.080 | 23.977 | 64.674 | 1.00 | 29.22 | B |
| ATOM | 1461 | N | GLY | 198 | 22.005 | 24.600 | 62.792 | 1.00 | 29.85 | B |
| ATOM | 1462 | CA | GLY | 198 | 23.141 | 25.345 | 62.275 | 1.00 | 30.66 | B |
| ATOM | 1463 | C | GLY | 198 | 24.040 | 24.637 | 61.282 | 1.00 | 30.74 | B |
| ATOM | 1464 | O | GLY | 198 | 24.857 | 25.283 | 60.618 | 1.00 | 30.16 | B |
| ATOM | 1465 | N | LEU | 199 | 23.903 | 23.318 | 61.178 | 1.00 | 30.32 | B |
| ATOM | 1466 | CA | LEU | 199 | 24.722 | 22.538 | 60.255 | 1.00 | 30.74 | B |
| ATOM | 1467 | CB | LEU | 199 | 24.530 | 21.004 | 60.530 | 1.00 | 30.24 | B |
| ATOM | 1468 | CG | LEU | 199 | 25.328 | 19.967 | 59.664 | 1.00 | 28.88 | B |
| ATOM | 1469 | CD1 | LEU | 199 | 26.773 | 20.398 | 59.527 | 1.00 | 30.22 | B |
| ATOM | 1470 | CD2 | LEU | 199 | 25.254 | 18.587 | 60.308 | 1.00 | 28.26 | B |
| ATOM | 1471 | C | LEU | 199 | 24.397 | 22.869 | 58.792 | 1.00 | 31.25 | B |
| ATOM | 1472 | O | LEU | 199 | 23.256 | 22.699 | 58.340 | 1.00 | 31.36 | B |
| ATOM | 1473 | N | GLU | 200 | 25.406 | 23.345 | 58.065 | 1.00 | 30.26 | B |
| ATOM | 1474 | CA | GLU | 200 | 25.253 | 23.712 | 56.661 | 1.00 | 32.06 | B |
| ATOM | 1475 | CB | GLU | 200 | 26.446 | 24.590 | 56.190 | 1.00 | 34.38 | B |
| ATOM | 1476 | CG | GLU | 200 | 26.604 | 25.870 | 56.961 | 1.00 | 41.33 | B |
| ATOM | 1477 | CD | GLU | 200 | 25.395 | 26.773 | 56.833 | 1.00 | 42.76 | B |
| ATOM | 1478 | OE1 | GLU | 200 | 25.121 | 27.535 | 57.785 | 1.00 | 43.19 | B |
| ATOM | 1479 | OE2 | GLU | 200 | 24.730 | 26.721 | 55.776 | 1.00 | 43.56 | B |
| ATOM | 1480 | C | GLU | 200 | 25.164 | 22.514 | 55.722 | 1.00 | 31.83 | B |
| ATOM | 1481 | O | GLU | 200 | 25.841 | 21.503 | 55.916 | 1.00 | 30.83 | B |
| ATOM | 1482 | N | GLU | 201 | 24.328 | 22.654 | 54.700 | 1.00 | 30.84 | B |
| ATOM | 1483 | CA | GLU | 201 | 24.163 | 21.639 | 53.677 | 1.00 | 30.37 | B |
| ATOM | 1484 | CB | GLU | 201 | 22.732 | 21.167 | 53.611 | 1.00 | 30.91 | B |
| ATOM | 1485 | CG | GLU | 201 | 22.386 | 20.111 | 54.629 | 1.00 | 33.83 | B |
| ATOM | 1486 | CD | GLU | 201 | 20.975 | 19.587 | 54.454 | 1.00 | 36.02 | B |
| ATOM | 1487 | OE1 | GLU | 201 | 20.052 | 20.163 | 55.069 | 1.00 | 37.16 | B |
| ATOM | 1488 | OE2 | GLU | 201 | 20.791 | 18.604 | 53.695 | 1.00 | 36.56 | B |
| ATOM | 1489 | C | GLU | 201 | 24.528 | 22.328 | 52.373 | 1.00 | 30.44 | B |
| ATOM | 1490 | O | GLU | 201 | 23.796 | 23.207 | 51.919 | 1.00 | 30.69 | B |
| ATOM | 1491 | N | ILE | 202 | 25.663 | 21.958 | 51.783 | 1.00 | 28.80 | B |
| ATOM | 1492 | CA | ILE | 202 | 26.073 | 22.575 | 50.526 | 1.00 | 28.82 | B |
| ATOM | 1493 | CB | ILE | 202 | 27.619 | 22.739 | 50.409 | 1.00 | 28.91 | B |
| ATOM | 1494 | CG2 | ILE | 202 | 27.978 | 23.225 | 49.014 | 1.00 | 26.00 | B |
| ATOM | 1495 | CG1 | ILE | 202 | 28.137 | 23.751 | 51.426 | 1.00 | 28.90 | B |
| ATOM | 1496 | CD1 | ILE | 202 | 28.057 | 23.294 | 52.863 | 1.00 | 32.03 | B |
| ATOM | 1497 | C | ILE | 202 | 25.594 | 21.773 | 49.324 | 1.00 | 28.57 | B |
| ATOM | 1498 | O | ILE | 202 | 25.844 | 20.571 | 49.215 | 1.00 | 29.93 | B |
| ATOM | 1499 | N | THR | 203 | 24.896 | 22.448 | 48.422 | 1.00 | 28.23 | B |
| ATOM | 1500 | CA | THR | 203 | 24.404 | 21.803 | 47.219 | 1.00 | 26.49 | B |
| ATOM | 1501 | CB | THR | 203 | 23.307 | 22.665 | 46.527 | 1.00 | 26.14 | B |
| ATOM | 1502 | OG1 | THR | 203 | 22.173 | 22.791 | 47.401 | 1.00 | 24.25 | B |
| ATOM | 1503 | CG2 | THR | 203 | 22.862 | 22.028 | 45.208 | 1.00 | 25.01 | B |
| ATOM | 1504 | C | THR | 203 | 25.606 | 21.636 | 46.293 | 1.00 | 26.13 | B |
| ATOM | 1505 | O | THR | 203 | 26.483 | 22.495 | 46.253 | 1.00 | 26.91 | B |
| ATOM | 1506 | N | VAL | 204 | 25.666 | 20.504 | 45.599 | 1.00 | 26.49 | B |
| ATOM | 1507 | CA | VAL | 204 | 26.741 | 20.220 | 44.654 | 1.00 | 27.51 | B |
| ATOM | 1508 | CB | VAL | 204 | 27.444 | 18.868 | 44.967 | 1.00 | 25.76 | B |
| ATOM | 1509 | CG1 | VAL | 204 | 28.653 | 18.672 | 44.056 | 1.00 | 23.12 | B |
| ATOM | 1510 | CG2 | VAL | 204 | 27.879 | 18.837 | 46.423 | 1.00 | 24.79 | B |
| ATOM | 1511 | C | VAL | 204 | 26.009 | 20.149 | 43.321 | 1.00 | 29.14 | B |
| ATOM | 1512 | O | VAL | 204 | 25.265 | 19.199 | 43.061 | 1.00 | 30.39 | B |
| ATOM | 1513 | N | HIS | 205 | 26.218 | 21.170 | 42.495 | 1.00 | 29.22 | B |
| ATOM | 1514 | CA | HIS | 205 | 25.553 | 21.313 | 41.195 | 1.00 | 30.55 | B |
| ATOM | 1515 | CB | HIS | 205 | 25.613 | 22.794 | 40.767 | 1.00 | 28.34 | B |
| ATOM | 1516 | CG | HIS | 205 | 25.157 | 23.732 | 41.838 | 1.00 | 28.46 | B |
| ATOM | 1517 | CD2 | HIS | 205 | 25.858 | 24.492 | 42.711 | 1.00 | 27.43 | B |
| ATOM | 1518 | ND1 | HIS | 205 | 23.832 | 23.862 | 42.196 | 1.00 | 28.83 | B |
| ATOM | 1519 | CE1 | HIS | 205 | 23.736 | 24.654 | 43.249 | 1.00 | 28.44 | B |
| ATOM | 1520 | NE2 | HIS | 205 | 24.952 | 25.049 | 43.582 | 1.00 | 29.92 | B |
| ATOM | 1521 | C | HIS | 205 | 26.092 | 20.435 | 40.081 | 1.00 | 31.51 | B |
| ATOM | 1522 | O | HIS | 205 | 25.358 | 20.055 | 39.169 | 1.00 | 31.34 | B |
| ATOM | 1523 | N | ASN | 206 | 27.383 | 20.136 | 40.147 | 1.00 | 33.49 | B |
| ATOM | 1524 | CA | ASN | 206 | 28.032 | 19.299 | 39.151 | 1.00 | 34.62 | B |
| ATOM | 1525 | CB | ASN | 206 | 28.444 | 20.138 | 37.930 | 1.00 | 34.75 | B |
| ATOM | 1526 | CG | ASN | 206 | 29.164 | 21.417 | 38.309 | 1.00 | 35.27 | B |
| ATOM | 1527 | OD1 | ASN | 206 | 30.224 | 21.391 | 38.938 | 1.00 | 37.58 | B |
| ATOM | 1528 | ND2 | ASN | 206 | 28.589 | 22.548 | 37.925 | 1.00 | 34.11 | B |
| ATOM | 1529 | C | ASN | 206 | 29.243 | 18.650 | 39.798 | 1.00 | 35.69 | B |
| ATOM | 1530 | O | ASN | 206 | 29.478 | 18.836 | 40.992 | 1.00 | 36.45 | B |
| ATOM | 1531 | N | LYS | 207 | 30.002 | 17.876 | 39.031 | 1.00 | 36.43 | B |
| ATOM | 1532 | CA | LYS | 207 | 31.171 | 17.216 | 39.590 | 1.00 | 38.62 | B |
| ATOM | 1533 | CB | LYS | 207 | 31.582 | 15.993 | 38.703 | 1.00 | 40.10 | B |
| ATOM | 1534 | CG | LYS | 207 | 32.123 | 16.339 | 37.319 | 1.00 | 42.56 | B |
| ATOM | 1535 | CD | LYS | 207 | 32.259 | 15.081 | 36.456 | 1.00 | 44.26 | B |
| ATOM | 1536 | CE | LYS | 207 | 33.191 | 15.293 | 35.267 | 1.00 | 43.78 | B |
| ATOM | 1537 | NZ | LYS | 207 | 34.613 | 15.454 | 35.696 | 1.00 | 42.46 | B |
| ATOM | 1538 | C | LYS | 207 | 32.313 | 18.222 | 39.700 | 1.00 | 39.03 | B |
| ATOM | 1539 | O | LYS | 207 | 33.176 | 18.120 | 40.576 | 1.00 | 38.73 | B |
| ATOM | 1540 | N | ASP | 208 | 32.292 | 19.208 | 38.813 | 1.00 | 39.88 | B |
| ATOM | 1541 | CA | ASP | 208 | 33.312 | 20.244 | 38.790 | 1.00 | 40.76 | B |
| ATOM | 1542 | CB | ASP | 208 | 33.248 | 20.981 | 37.461 | 1.00 | 42.58 | B |
| ATOM | 1543 | CG | ASP | 208 | 33.659 | 20.101 | 36.292 | 1.00 | 45.91 | B |
| ATOM | 1544 | OD1 | ASP | 208 | 33.407 | 20.484 | 35.127 | 1.00 | 46.74 | B |
| ATOM | 1545 | OD2 | ASP | 208 | 34.246 | 19.023 | 36.542 | 1.00 | 46.78 | B |
| ATOM | 1546 | C | ASP | 208 | 33.141 | 21.219 | 39.952 | 1.00 | 39.55 | B |
| ATOM | 1547 | O | ASP | 208 | 33.643 | 22.339 | 39.922 | 1.00 | 41.22 | B |
| ATOM | 1548 | N | GLU | 209 | 32.457 | 20.784 | 40.996 | 1.00 | 37.46 | B |
| ATOM | 1549 | CA | GLU | 209 | 32.241 | 21.660 | 42.128 | 1.00 | 35.89 | B |
| ATOM | 1550 | CB | GLU | 209 | 30.760 | 22.075 | 42.158 | 1.00 | 35.84 | B |
| ATOM | 1551 | CG | GLU | 209 | 30.445 | 23.275 | 43.010 | 1.00 | 37.17 | B |
| ATOM | 1552 | CD | GLU | 209 | 28.973 | 23.682 | 42.924 | 1.00 | 38.94 | B |
| ATOM | 1553 | OE1 | GLU | 209 | 28.462 | 23.857 | 41.793 | 1.00 | 37.72 | B |
| ATOM | 1554 | OE2 | GLU | 209 | 28.327 | 23.835 | 43.988 | 1.00 | 38.77 | B |
| ATOM | 1555 | C | GLU | 209 | 32.646 | 20.992 | 43.439 | 1.00 | 34.61 | B |
| ATOM | 1556 | O | GLU | 209 | 32.763 | 21.657 | 44.470 | 1.00 | 36.51 | B |
| ATOM | 1557 | N | VAL | 210 | 32.907 | 19.690 | 43.395 | 1.00 | 32.07 | B |
| ATOM | 1558 | CA | VAL | 210 | 33.268 | 18.966 | 44.609 | 1.00 | 29.92 | B |
| ATOM | 1559 | CB | VAL | 210 | 33.065 | 17.411 | 44.450 | 1.00 | 29.01 | B |
| ATOM | 1560 | CG1 | VAL | 210 | 31.856 | 17.110 | 43.574 | 1.00 | 26.09 | B |
| ATOM | 1561 | CG2 | VAL | 210 | 34.301 | 16.774 | 43.901 | 1.00 | 29.03 | B |
| ATOM | 1562 | C | VAL | 210 | 34.668 | 19.212 | 45.183 | 1.00 | 28.45 | B |
| ATOM | 1563 | O | VAL | 210 | 34.820 | 19.322 | 46.406 | 1.00 | 29.31 | B |
| ATOM | 1564 | N | TYR | 211 | 35.694 | 19.311 | 44.343 | 1.00 | 26.40 | B |
| ATOM | 1565 | CA | TYR | 211 | 37.038 | 19.505 | 44.894 | 1.00 | 24.93 | B |
| ATOM | 1566 | CB | TYR | 211 | 38.106 | 19.552 | 43.783 | 1.00 | 22.02 | B |
| ATOM | 1567 | CG | TYR | 211 | 39.510 | 19.386 | 44.318 | 1.00 | 23.83 | B |
| ATOM | 1568 | CD1 | TYR | 211 | 39.850 | 18.284 | 45.097 | 1.00 | 26.06 | B |
| ATOM | 1569 | CE1 | TYR | 211 | 41.136 | 18.131 | 45.625 | 1.00 | 25.76 | B |
| ATOM | 1570 | CD2 | TYR | 211 | 40.498 | 20.339 | 44.074 | 1.00 | 24.90 | B |
| ATOM | 1571 | CE2 | TYR | 211 | 41.790 | 20.196 | 44.597 | 1.00 | 24.81 | B |
| ATOM | 1572 | CZ | TYR | 211 | 42.103 | 19.089 | 45.374 | 1.00 | 25.75 | B |
| ATOM | 1573 | OH | TYR | 211 | 43.373 | 18.938 | 45.910 | 1.00 | 23.97 | B |
| ATOM | 1574 | C | TYR | 211 | 37.111 | 20.759 | 45.757 | 1.00 | 25.45 | B |
| ATOM | 1575 | O | TYR | 211 | 37.691 | 20.740 | 46.844 | 1.00 | 24.21 | B |
| ATOM | 1576 | N | GLN | 212 | 36.501 | 21.840 | 45.272 | 1.00 | 27.99 | B |
| ATOM | 1577 | CA | GLN | 212 | 36.473 | 23.117 | 45.983 | 1.00 | 27.45 | B |
| ATOM | 1578 | CB | GLN | 212 | 35.721 | 24.126 | 45.163 | 1.00 | 31.66 | B |
| ATOM | 1579 | CG | GLN | 212 | 35.365 | 25.402 | 45.907 | 1.00 | 37.63 | B |
| ATOM | 1580 | CD | GLN | 212 | 35.696 | 26.654 | 45.105 | 1.00 | 40.53 | B |
| ATOM | 1581 | OE1 | GLN | 212 | 35.305 | 26.782 | 43.937 | 1.00 | 39.59 | B |
| ATOM | 1582 | NE2 | GLN | 212 | 36.418 | 27.587 | 45.731 | 1.00 | 39.73 | B |
| ATOM | 1583 | C | GLN | 212 | 35.834 | 22.981 | 47.364 | 1.00 | 26.73 | B |
| ATOM | 1584 | O | GLN | 212 | 36.329 | 23.527 | 48.347 | 1.00 | 26.01 | B |
| ATOM | 1585 | N | ILE | 213 | 34.733 | 22.243 | 47.437 | 1.00 | 26.10 | B |
| ATOM | 1586 | CA | ILE | 213 | 34.044 | 22.037 | 48.703 | 1.00 | 24.91 | B |
| ATOM | 1587 | CB | ILE | 213 | 32.694 | 21.327 | 48.496 | 1.00 | 23.51 | B |
| ATOM | 1588 | CG2 | ILE | 213 | 31.978 | 21.200 | 49.835 | 1.00 | 20.39 | B |
| ATOM | 1589 | CG1 | ILE | 213 | 31.843 | 22.117 | 47.461 | 1.00 | 22.89 | B |
| ATOM | 1590 | CD1 | ILE | 213 | 30.472 | 21.509 | 47.152 | 1.00 | 23.13 | B |
| ATOM | 1591 | C | ILE | 213 | 34.906 | 21.207 | 49.656 | 1.00 | 25.49 | B |
| ATOM | 1592 | O | ILE | 213 | 34.916 | 21.448 | 50.865 | 1.00 | 24.30 | B |
| ATOM | 1593 | N | LEU | 214 | 35.618 | 20.226 | 49.106 | 1.00 | 26.92 | B |
| ATOM | 1594 | CA | LEU | 214 | 36.496 | 19.381 | 49.905 | 1.00 | 28.08 | B |
| ATOM | 1595 | CB | LEU | 214 | 37.031 | 18.168 | 49.050 | 1.00 | 28.21 | B |
| ATOM | 1596 | CG | LEU | 214 | 36.272 | 16.802 | 49.152 | 1.00 | 30.13 | B |
| ATOM | 1597 | CD1 | LEU | 214 | 34.796 | 17.034 | 49.411 | 1.00 | 31.20 | B |
| ATOM | 1598 | CD2 | LEU | 214 | 36.482 | 15.987 | 47.876 | 1.00 | 29.12 | B |
| ATOM | 1599 | C | LEU | 214 | 37.657 | 20.225 | 50.442 | 1.00 | 29.28 | B |
| ATOM | 1600 | O | LEU | 214 | 38.012 | 20.114 | 51.620 | 1.00 | 30.45 | B |
| ATOM | 1601 | N | GLU | 215 | 38.235 | 21.083 | 49.599 | 1.00 | 28.08 | B |
| ATOM | 1602 | CA | GLU | 215 | 39.339 | 21.932 | 50.059 | 1.00 | 28.89 | B |
| ATOM | 1603 | CB | GLU | 215 | 39.864 | 22.842 | 48.914 | 1.00 | 29.69 | B |
| ATOM | 1604 | CG | GLU | 215 | 40.426 | 22.093 | 47.714 | 1.00 | 33.51 | B |
| ATOM | 1605 | CD | GLU | 215 | 41.092 | 23.014 | 46.700 | 1.00 | 36.27 | B |
| ATOM | 1606 | OE1 | GLU | 215 | 42.343 | 23.136 | 46.730 | 1.00 | 34.34 | B |
| ATOM | 1607 | OE2 | GLU | 215 | 40.358 | 23.620 | 45.880 | 1.00 | 36.57 | B |
| ATOM | 1608 | C | GLU | 215 | 38.919 | 22.795 | 51.255 | 1.00 | 28.03 | B |
| ATOM | 1609 | O | GLU | 215 | 39.682 | 22.953 | 52.210 | 1.00 | 27.31 | B |
| ATOM | 1610 | N | LYS | 216 | 37.707 | 23.348 | 51.204 | 1.00 | 27.99 | B |
| ATOM | 1611 | CA | LYS | 216 | 37.202 | 24.183 | 52.290 | 1.00 | 29.52 | B |
| ATOM | 1612 | CB | LYS | 216 | 35.799 | 24.696 | 51.971 | 1.00 | 30.11 | B |
| ATOM | 1613 | CG | LYS | 216 | 35.691 | 25.416 | 50.650 | 1.00 | 32.53 | B |
| ATOM | 1614 | CD | LYS | 216 | 36.584 | 26.643 | 50.602 | 1.00 | 34.31 | B |
| ATOM | 1615 | CE | LYS | 216 | 36.596 | 27.272 | 49.200 | 1.00 | 36.64 | B |
| ATOM | 1616 | NZ | LYS | 216 | 37.248 | 26.419 | 48.152 | 1.00 | 34.44 | B |
| ATOM | 1617 | C | LYS | 216 | 37.170 | 23.415 | 53.609 | 1.00 | 30.05 | B |
| ATOM | 1618 | O | LYS | 216 | 37.516 | 23.960 | 54.658 | 1.00 | 31.96 | B |
| ATOM | 1619 | N | GLY | 217 | 36.742 | 22.156 | 53.553 | 1.00 | 30.83 | B |
| ATOM | 1620 | CA | GLY | 217 | 36.695 | 21.335 | 54.752 | 1.00 | 29.82 | B |
| ATOM | 1621 | C | GLY | 217 | 38.107 | 21.144 | 55.270 | 1.00 | 29.77 | B |
| ATOM | 1622 | O | GLY | 217 | 38.389 | 21.354 | 56.460 | 1.00 | 28.73 | B |
| ATOM | 1623 | N | ALA | 218 | 39.000 | 20.749 | 54.363 | 1.00 | 29.20 | B |
| ATOM | 1624 | CA | ALA | 218 | 40.404 | 20.548 | 54.696 | 1.00 | 28.09 | B |
| ATOM | 1625 | CB | ALA | 218 | 41.212 | 20.299 | 53.427 | 1.00 | 25.39 | B |
| ATOM | 1626 | C | ALA | 218 | 40.924 | 21.792 | 55.422 | 1.00 | 27.61 | B |
| ATOM | 1627 | O | ALA | 218 | 41.623 | 21.684 | 56.429 | 1.00 | 27.17 | B |
| ATOM | 1628 | N | ALA | 219 | 40.559 | 22.969 | 54.914 | 1.00 | 27.54 | B |
| ATOM | 1629 | CA | ALA | 219 | 40.984 | 24.243 | 55.505 | 1.00 | 27.45 | B |
| ATOM | 1630 | CB | ALA | 219 | 40.430 | 25.406 | 54.695 | 1.00 | 26.20 | B |
| ATOM | 1631 | C | ALA | 219 | 40.553 | 24.385 | 56.964 | 1.00 | 27.16 | B |
| ATOM | 1632 | O | ALA | 219 | 41.368 | 24.726 | 57.833 | 1.00 | 26.05 | B |
| ATOM | 1633 | N | LYS | 220 | 39.273 | 24.135 | 57.227 | 1.00 | 26.17 | B |
| ATOM | 1634 | CA | LYS | 220 | 38.754 | 24.234 | 58.585 | 1.00 | 26.59 | B |
| ATOM | 1635 | CB | LYS | 220 | 37.203 | 24.057 | 58.592 | 1.00 | 25.82 | B |
| ATOM | 1636 | CG | LYS | 220 | 36.477 | 25.037 | 57.691 | 1.00 | 26.36 | B |
| ATOM | 1637 | CD | LYS | 220 | 34.997 | 25.195 | 58.065 | 1.00 | 28.61 | B |
| ATOM | 1638 | CE | LYS | 220 | 34.827 | 25.771 | 59.471 | 1.00 | 27.13 | B |
| ATOM | 1639 | NZ | LYS | 220 | 33.406 | 26.129 | 59.789 | 1.00 | 25.98 | B |
| ATOM | 1640 | C | LYS | 220 | 39.426 | 23.190 | 59.491 | 1.00 | 26.00 | B |
| ATOM | 1641 | O | LYS | 220 | 39.715 | 23.465 | 60.665 | 1.00 | 24.88 | B |
| ATOM | 1642 | N | ARG | 221 | 39.671 | 22.000 | 58.937 | 1.00 | 24.80 | B |
| ATOM | 1643 | CA | ARG | 221 | 40.330 | 20.916 | 59.671 | 1.00 | 22.73 | B |
| ATOM | 1644 | CB | ARG | 221 | 40.685 | 19.757 | 58.725 | 1.00 | 24.70 | B |
| ATOM | 1645 | CG | ARG | 221 | 39.524 | 18.885 | 58.293 | 1.00 | 25.62 | B |
| ATOM | 1646 | CD | ARG | 221 | 39.367 | 17.736 | 59.256 | 1.00 | 26.10 | B |
| ATOM | 1647 | NE | ARG | 221 | 38.190 | 16.934 | 58.960 | 1.00 | 24.76 | B |
| ATOM | 1648 | CZ | ARG | 221 | 38.065 | 16.146 | 57.901 | 1.00 | 22.87 | B |
| ATOM | 1649 | NH1 | ARG | 221 | 39.061 | 16.051 | 57.021 | 1.00 | 19.50 | B |
| ATOM | 1650 | NH2 | ARG | 221 | 36.942 | 15.451 | 57.735 | 1.00 | 20.09 | B |
| ATOM | 1651 | C | ARG | 221 | 41.624 | 21.456 | 60.267 | 1.00 | 21.95 | B |
| ATOM | 1652 | O | ARG | 221 | 41.889 | 21.306 | 61.466 | 1.00 | 20.88 | B |
| ATOM | 1653 | N | THR | 222 | 42.421 | 22.089 | 59.406 | 1.00 | 20.21 | B |
| ATOM | 1654 | CA | THR | 222 | 43.705 | 22.661 | 59.795 | 1.00 | 19.39 | B |
| ATOM | 1655 | CB | THR | 222 | 44.312 | 23.464 | 58.650 | 1.00 | 21.09 | B |
| ATOM | 1656 | OG1 | THR | 222 | 44.502 | 22.600 | 57.525 | 1.00 | 22.38 | B |
| ATOM | 1657 | CG2 | THR | 222 | 45.649 | 24.077 | 59.073 | 1.00 | 20.44 | B |
| ATOM | 1658 | C | THR | 222 | 43.589 | 23.579 | 60.991 | 1.00 | 18.28 | B |
| ATOM | 1659 | O | THR | 222 | 44.338 | 23.441 | 61.952 | 1.00 | 17.80 | B |
| ATOM | 1660 | N | THR | 223 | 42.649 | 24.517 | 60.926 | 1.00 | 17.37 | B |
| ATOM | 1661 | CA | THR | 223 | 42.452 | 25.461 | 62.012 | 1.00 | 18.66 | B |
| ATOM | 1662 | CB | THR | 223 | 41.496 | 26.590 | 61.605 | 1.00 | 17.71 | B |
| ATOM | 1663 | OG1 | THR | 223 | 40.245 | 26.413 | 62.268 | 1.00 | 20.08 | B |
| ATOM | 1664 | CG2 | THR | 223 | 41.258 | 26.581 | 60.111 | 1.00 | 16.54 | B |
| ATOM | 1665 | C | THR | 223 | 41.902 | 24.740 | 63.242 | 1.00 | 20.76 | B |
| ATOM | 1666 | O | THR | 223 | 42.206 | 25.120 | 64.374 | 1.00 | 24.08 | B |
| ATOM | 1667 | N | ALA | 224 | 41.100 | 23.698 | 63.018 | 1.00 | 21.47 | B |
| ATOM | 1668 | CA | ALA | 224 | 40.529 | 22.898 | 64.105 | 1.00 | 19.87 | B |
| ATOM | 1669 | CB | ALA | 224 | 39.642 | 21.801 | 63.534 | 1.00 | 22.14 | B |
| ATOM | 1670 | C | ALA | 224 | 41.667 | 22.266 | 64.894 | 1.00 | 19.87 | B |
| ATOM | 1671 | O | ALA | 224 | 41.689 | 22.289 | 66.129 | 1.00 | 16.71 | B |
| ATOM | 1672 | N | ALA | 225 | 42.604 | 21.680 | 64.155 | 1.00 | 20.37 | B |
| ATOM | 1673 | CA | ALA | 225 | 43.765 | 21.048 | 64.755 | 1.00 | 20.88 | B |
| ATOM | 1674 | CB | ALA | 225 | 44.647 | 20.440 | 63.666 | 1.00 | 19.50 | B |
| ATOM | 1675 | C | ALA | 225 | 44.541 | 22.096 | 65.553 | 1.00 | 22.18 | B |
| ATOM | 1676 | O | ALA | 225 | 45.054 | 21.808 | 66.638 | 1.00 | 20.94 | B |
| ATOM | 1677 | N | THR | 226 | 44.613 | 23.319 | 65.023 | 1.00 | 23.92 | B |
| ATOM | 1678 | CA | THR | 226 | 45.324 | 24.401 | 65.717 | 1.00 | 24.83 | B |
| ATOM | 1679 | CB | THR | 226 | 45.313 | 25.723 | 64.895 | 1.00 | 24.59 | B |
| ATOM | 1680 | OG1 | THR | 226 | 46.088 | 25.565 | 63.699 | 1.00 | 23.18 | B |
| ATOM | 1681 | CG2 | THR | 226 | 45.904 | 26.866 | 65.721 | 1.00 | 25.23 | B |
| ATOM | 1682 | C | THR | 226 | 44.699 | 24.679 | 67.089 | 1.00 | 25.41 | B |
| ATOM | 1683 | O | THR | 226 | 45.405 | 24.877 | 68.083 | 1.00 | 25.12 | B |
| ATOM | 1684 | N | LEU | 227 | 43.370 | 24.680 | 67.130 | 1.00 | 25.47 | B |
| ATOM | 1685 | CA | LEU | 227 | 42.619 | 24.942 | 68.353 | 1.00 | 26.90 | B |
| ATOM | 1686 | CB | LEU | 227 | 41.222 | 25.541 | 67.980 | 1.00 | 29.00 | B |
| ATOM | 1687 | CG | LEU | 227 | 41.051 | 27.041 | 67.561 | 1.00 | 32.68 | B |
| ATOM | 1688 | CD1 | LEU | 227 | 42.240 | 27.567 | 66.763 | 1.00 | 31.51 | B |
| ATOM | 1689 | CD2 | LEU | 227 | 39.756 | 27.156 | 66.755 | 1.00 | 32.75 | B |
| ATOM | 1690 | C | LEU | 227 | 42.409 | 23.739 | 69.296 | 1.00 | 26.44 | B |
| ATOM | 1691 | O | LEU | 227 | 42.348 | 23.906 | 70.520 | 1.00 | 25.50 | B |
| ATOM | 1692 | N | MET | 228 | 42.295 | 22.533 | 68.755 | 1.00 | 24.99 | B |
| ATOM | 1693 | CA | MET | 228 | 42.041 | 21.392 | 69.635 | 1.00 | 25.58 | B |
| ATOM | 1694 | CB | MET | 228 | 40.625 | 20.786 | 69.310 | 1.00 | 27.00 | B |
| ATOM | 1695 | CG | MET | 228 | 39.499 | 21.798 | 69.554 | 1.00 | 28.30 | B |
| ATOM | 1696 | SD | MET | 228 | 37.874 | 21.368 | 68.919 | 1.00 | 31.74 | B |
| ATOM | 1697 | CE | MET | 228 | 37.998 | 22.026 | 67.265 | 1.00 | 30.21 | B |
| ATOM | 1698 | C | MET | 228 | 43.091 | 20.301 | 69.666 | 1.00 | 23.55 | B |
| ATOM | 1699 | O | MET | 228 | 43.547 | 19.828 | 68.629 | 1.00 | 23.83 | B |
| ATOM | 1700 | N | ASN | 229 | 43.471 | 19.913 | 70.882 | 1.00 | 22.85 | B |
| ATOM | 1701 | CA | ASN | 229 | 44.470 | 18.870 | 71.099 | 1.00 | 21.02 | B |
| ATOM | 1702 | CB | ASN | 229 | 44.574 | 18.524 | 72.588 | 1.00 | 19.32 | B |
| ATOM | 1703 | CG | ASN | 229 | 45.172 | 19.646 | 73.426 | 1.00 | 19.33 | B |
| ATOM | 1704 | OD1 | ASN | 229 | 45.690 | 20.634 | 72.899 | 1.00 | 19.44 | B |
| ATOM | 1705 | ND2 | ASN | 229 | 45.112 | 19.484 | 74.751 | 1.00 | 13.92 | B |
| ATOM | 1706 | C | ASN | 229 | 44.162 | 17.582 | 70.329 | 1.00 | 21.09 | B |
| ATOM | 1707 | O | ASN | 229 | 43.063 | 17.026 | 70.435 | 1.00 | 21.09 | B |
| ATOM | 1708 | N | ALA | 230 | 45.144 | 17.121 | 69.558 | 1.00 | 20.25 | B |
| ATOM | 1709 | CA | ALA | 230 | 45.030 | 15.887 | 68.786 | 1.00 | 19.42 | B |
| ATOM | 1710 | CB | ALA | 230 | 45.224 | 14.675 | 69.721 | 1.00 | 21.67 | B |
| ATOM | 1711 | C | ALA | 230 | 43.694 | 15.783 | 68.067 | 1.00 | 18.26 | B |
| ATOM | 1712 | O | ALA | 230 | 43.096 | 14.712 | 68.000 | 1.00 | 17.83 | B |
| ATOM | 1713 | N | TYR | 231 | 43.242 | 16.897 | 67.512 | 1.00 | 17.17 | B |
| ATOM | 1714 | CA | TYR | 231 | 41.965 | 16.927 | 66.821 | 1.00 | 17.72 | B |
| ATOM | 1715 | CB | TYR | 231 | 41.694 | 18.379 | 66.201 | 1.00 | 15.95 | B |
| ATOM | 1716 | CG | TYR | 231 | 40.341 | 18.465 | 65.524 | 1.00 | 12.55 | B |
| ATOM | 1717 | CD1 | TYR | 231 | 40.205 | 18.269 | 64.151 | 1.00 | 12.28 | B |
| ATOM | 1718 | CE1 | TYR | 231 | 38.933 | 18.219 | 63.555 | 1.00 | 8.18 | B |
| ATOM | 1719 | CD2 | TYR | 231 | 39.182 | 18.621 | 66.279 | 1.00 | 10.61 | B |
| ATOM | 1720 | CE2 | TYR | 231 | 37.918 | 18.573 | 65.690 | 1.00 | 9.26 | B |
| ATOM | 1721 | CZ | TYR | 231 | 37.802 | 18.372 | 64.338 | 1.00 | 6.19 | B |
| ATOM | 1722 | OH | TYR | 231 | 36.545 | 18.335 | 63.777 | 1.00 | 8.98 | B |
| ATOM | 1723 | C | TYR | 231 | 41.728 | 15.869 | 65.731 | 1.00 | 18.14 | B |
| ATOM | 1724 | O | TYR | 231 | 40.596 | 15.392 | 65.571 | 1.00 | 17.92 | B |
| ATOM | 1725 | N | SER | 232 | 42.769 | 15.504 | 64.982 | 1.00 | 17.34 | B |
| ATOM | 1726 | CA | SER | 232 | 42.585 | 14.537 | 63.903 | 1.00 | 17.96 | B |
| ATOM | 1727 | CB | SER | 232 | 43.681 | 14.688 | 62.816 | 1.00 | 13.72 | B |
| ATOM | 1728 | OG | SER | 232 | 44.941 | 14.251 | 63.275 | 1.00 | 15.73 | B |
| ATOM | 1729 | C | SER | 232 | 42.502 | 13.070 | 64.323 | 1.00 | 18.78 | B |
| ATOM | 1730 | O | SER | 232 | 41.934 | 12.255 | 63.598 | 1.00 | 19.24 | B |
| ATOM | 1731 | N | SER | 233 | 43.051 | 12.726 | 65.480 | 1.00 | 17.77 | B |
| ATOM | 1732 | CA | SER | 233 | 43.019 | 11.340 | 65.904 | 1.00 | 16.56 | B |
| ATOM | 1733 | CB | SER | 233 | 44.383 | 10.932 | 66.496 | 1.00 | 18.00 | B |
| ATOM | 1734 | OG | SER | 233 | 44.509 | 11.362 | 67.846 | 1.00 | 17.89 | B |
| ATOM | 1735 | C | SER | 233 | 41.935 | 11.141 | 66.943 | 1.00 | 17.20 | B |
| ATOM | 1736 | O | SER | 233 | 41.413 | 10.035 | 67.110 | 1.00 | 13.55 | B |
| ATOM | 1737 | N | ARG | 234 | 41.570 | 12.235 | 67.609 | 1.00 | 18.37 | B |
| ATOM | 1738 | CA | ARG | 234 | 40.579 | 12.185 | 68.678 | 1.00 | 18.14 | B |
| ATOM | 1739 | CB | ARG | 234 | 41.035 | 13.079 | 69.848 | 1.00 | 20.04 | B |
| ATOM | 1740 | CG | ARG | 234 | 41.136 | 12.352 | 71.169 | 1.00 | 23.36 | B |
| ATOM | 1741 | CD | ARG | 234 | 42.547 | 12.392 | 71.767 | 1.00 | 25.39 | B |
| ATOM | 1742 | NE | ARG | 234 | 42.847 | 13.651 | 72.455 | 1.00 | 28.46 | B |
| ATOM | 1743 | CZ | ARG | 234 | 43.898 | 13.844 | 73.255 | 1.00 | 28.83 | B |
| ATOM | 1744 | NH1 | ARG | 234 | 44.765 | 12.865 | 73.479 | 1.00 | 28.24 | B |
| ATOM | 1745 | NH2 | ARG | 234 | 44.082 | 15.019 | 73.842 | 1.00 | 28.56 | B |
| ATOM | 1746 | C | ARG | 234 | 39.142 | 12.524 | 68.318 | 1.00 | 17.12 | B |
| ATOM | 1747 | O | ARG | 234 | 38.262 | 12.440 | 69.174 | 1.00 | 16.45 | B |
| ATOM | 1748 | N | SER | 235 | 38.879 | 12.876 | 67.064 | 1.00 | 17.25 | B |
| ATOM | 1749 | CA | SER | 235 | 37.508 | 13.232 | 66.685 | 1.00 | 17.01 | B |
| ATOM | 1750 | CB | SER | 235 | 37.470 | 14.581 | 66.108 | 1.00 | 16.15 | B |
| ATOM | 1751 | OG | SER | 235 | 38.109 | 14.594 | 64.847 | 1.00 | 15.24 | B |
| ATOM | 1752 | C | SER | 235 | 36.847 | 12.297 | 65.697 | 1.00 | 17.23 | B |
| ATOM | 1753 | O | SER | 235 | 37.505 | 11.536 | 64.991 | 1.00 | 17.87 | B |
| ATOM | 1754 | N | HIS | 236 | 35.527 | 12.381 | 65.655 | 1.00 | 16.90 | B |
| ATOM | 1755 | CA | HIS | 236 | 34.720 | 11.580 | 64.750 | 1.00 | 18.47 | B |
| ATOM | 1756 | CB | HIS | 236 | 33.553 | 10.961 | 65.484 | 1.00 | 20.05 | B |
| ATOM | 1757 | CG | HIS | 236 | 33.941 | 10.192 | 66.705 | 1.00 | 21.39 | B |
| ATOM | 1758 | CD2 | HIS | 236 | 33.907 | 10.529 | 68.016 | 1.00 | 20.87 | B |
| ATOM | 1759 | ND1 | HIS | 236 | 34.444 | 8.910 | 66.650 | 1.00 | 21.00 | B |
| ATOM | 1760 | CE1 | HIS | 236 | 34.700 | 8.490 | 67.876 | 1.00 | 20.80 | B |
| ATOM | 1761 | NE2 | HIS | 236 | 34.385 | 9.454 | 68.723 | 1.00 | 19.15 | B |
| ATOM | 1762 | C | HIS | 236 | 34.166 | 12.518 | 63.688 | 1.00 | 19.93 | B |
| ATOM | 1763 | O | HIS | 236 | 33.598 | 13.569 | 64.005 | 1.00 | 18.38 | B |
| ATOM | 1764 | N | SER | 237 | 34.326 | 12.155 | 62.425 | 1.00 | 20.64 | B |
| ATOM | 1765 | CA | SER | 237 | 33.795 | 13.001 | 61.374 | 1.00 | 21.44 | B |
| ATOM | 1766 | CB | SER | 237 | 34.889 | 13.424 | 60.424 | 1.00 | 20.37 | B |
| ATOM | 1767 | OG | SER | 237 | 35.258 | 12.370 | 59.566 | 1.00 | 19.17 | B |
| ATOM | 1768 | C | SER | 237 | 32.731 | 12.224 | 60.619 | 1.00 | 21.91 | B |
| ATOM | 1769 | O | SER | 237 | 32.908 | 11.043 | 60.320 | 1.00 | 21.18 | B |
| ATOM | 1770 | N | VAL | 238 | 31.620 | 12.886 | 60.324 | 1.00 | 21.76 | B |
| ATOM | 1771 | CA | VAL | 238 | 30.548 | 12.246 | 59.587 | 1.00 | 22.83 | B |
| ATOM | 1772 | CB | VAL | 238 | 29.297 | 12.024 | 60.475 | 1.00 | 25.08 | B |
| ATOM | 1773 | CG1 | VAL | 238 | 29.043 | 13.241 | 61.323 | 1.00 | 27.25 | B |
| ATOM | 1774 | CG2 | VAL | 238 | 28.077 | 11.717 | 59.601 | 1.00 | 24.91 | B |
| ATOM | 1775 | C | VAL | 238 | 30.176 | 13.052 | 58.366 | 1.00 | 21.64 | B |
| ATOM | 1776 | O | VAL | 238 | 29.399 | 13.986 | 58.450 | 1.00 | 24.16 | B |
| ATOM | 1777 | N | PHE | 239 | 30.764 | 12.683 | 57.232 | 1.00 | 23.48 | B |
| ATOM | 1778 | CA | PHE | 239 | 30.513 | 13.331 | 55.943 | 1.00 | 23.45 | B |
| ATOM | 1779 | CB | PHE | 239 | 31.736 | 13.139 | 55.002 | 1.00 | 22.63 | B |
| ATOM | 1780 | CG | PHE | 239 | 31.658 | 13.923 | 53.722 | 1.00 | 20.75 | B |
| ATOM | 1781 | CD1 | PHE | 239 | 30.660 | 13.667 | 52.785 | 1.00 | 19.42 | B |
| ATOM | 1782 | CD2 | PHE | 239 | 32.580 | 14.928 | 53.458 | 1.00 | 20.63 | B |
| ATOM | 1783 | CE1 | PHE | 239 | 30.578 | 14.403 | 51.596 | 1.00 | 21.05 | B |
| ATOM | 1784 | CE2 | PHE | 239 | 32.510 | 15.676 | 52.268 | 1.00 | 21.14 | B |
| ATOM | 1785 | CZ | PHE | 239 | 31.506 | 15.413 | 51.334 | 1.00 | 19.84 | B |
| ATOM | 1786 | C | PHE | 239 | 29.286 | 12.669 | 55.321 | 1.00 | 24.62 | B |
| ATOM | 1787 | O | PHE | 239 | 29.326 | 11.482 | 54.983 | 1.00 | 24.57 | B |
| ATOM | 1788 | N | SER | 240 | 28.202 | 13.430 | 55.178 | 1.00 | 24.38 | B |
| ATOM | 1789 | CA | SER | 240 | 26.968 | 12.910 | 54.596 | 1.00 | 23.26 | B |
| ATOM | 1790 | CB | SER | 240 | 25.778 | 13.249 | 55.480 | 1.00 | 22.32 | B |
| ATOM | 1791 | OG | SER | 240 | 25.932 | 12.724 | 56.786 | 1.00 | 21.48 | B |
| ATOM | 1792 | C | SER | 240 | 26.704 | 13.447 | 53.199 | 1.00 | 23.92 | B |
| ATOM | 1793 | O | SER | 240 | 27.065 | 14.568 | 52.865 | 1.00 | 23.73 | B |
| ATOM | 1794 | N | VAL | 241 | 26.067 | 12.622 | 52.382 | 1.00 | 25.40 | B |
| ATOM | 1795 | CA | VAL | 241 | 25.712 | 12.995 | 51.022 | 1.00 | 25.45 | B |
| ATOM | 1796 | CB | VAL | 241 | 26.654 | 12.349 | 49.985 | 1.00 | 26.85 | B |
| ATOM | 1797 | CG1 | VAL | 241 | 26.790 | 10.856 | 50.249 | 1.00 | 26.88 | B |
| ATOM | 1798 | CG2 | VAL | 241 | 26.118 | 12.595 | 48.579 | 1.00 | 26.95 | B |
| ATOM | 1799 | C | VAL | 241 | 24.293 | 12.513 | 50.787 | 1.00 | 25.56 | B |
| ATOM | 1800 | O | VAL | 241 | 24.013 | 11.321 | 50.856 | 1.00 | 25.33 | B |
| ATOM | 1801 | N | THR | 242 | 23.391 | 13.454 | 50.536 | 1.00 | 26.85 | B |
| ATOM | 1802 | CA | THR | 242 | 21.996 | 13.130 | 50.302 | 1.00 | 26.02 | B |
| ATOM | 1803 | CB | THR | 242 | 21.091 | 13.997 | 51.182 | 1.00 | 26.36 | B |
| ATOM | 1804 | OG1 | THR | 242 | 21.447 | 13.814 | 52.557 | 1.00 | 26.94 | B |
| ATOM | 1805 | CG2 | THR | 242 | 19.628 | 13.612 | 50.995 | 1.00 | 28.00 | B |
| ATOM | 1806 | C | THR | 242 | 21.656 | 13.352 | 48.832 | 1.00 | 27.35 | B |
| ATOM | 1807 | O | THR | 242 | 22.126 | 14.311 | 48.217 | 1.00 | 26.21 | B |
| ATOM | 1808 | N | ILE | 243 | 20.857 | 12.451 | 48.263 | 1.00 | 28.40 | B |
| ATOM | 1809 | CA | ILE | 243 | 20.468 | 12.564 | 46.861 | 1.00 | 28.65 | B |
| ATOM | 1810 | CB | ILE | 243 | 21.048 | 11.407 | 46.017 | 1.00 | 28.29 | B |
| ATOM | 1811 | CG2 | ILE | 243 | 20.944 | 11.746 | 44.534 | 1.00 | 27.94 | B |
| ATOM | 1812 | CG1 | ILE | 243 | 22.526 | 11.156 | 46.392 | 1.00 | 29.06 | B |
| ATOM | 1813 | CD1 | ILE | 243 | 23.191 | 10.046 | 45.592 | 1.00 | 25.36 | B |
| ATOM | 1814 | C | ILE | 243 | 18.950 | 12.538 | 46.721 | 1.00 | 29.68 | B |
| ATOM | 1815 | O | ILE | 243 | 18.327 | 11.512 | 46.966 | 1.00 | 30.63 | B |
| ATOM | 1816 | N | HIS | 244 | 18.355 | 13.672 | 46.358 | 1.00 | 31.77 | B |
| ATOM | 1817 | CA | HIS | 244 | 16.908 | 13.744 | 46.158 | 1.00 | 32.56 | B |
| ATOM | 1818 | CB | HIS | 244 | 16.354 | 15.175 | 46.421 | 1.00 | 33.70 | B |
| ATOM | 1819 | CG | HIS | 244 | 16.323 | 15.570 | 47.864 | 1.00 | 34.78 | B |
| ATOM | 1820 | CD2 | HIS | 244 | 15.331 | 15.500 | 48.785 | 1.00 | 35.77 | B |
| ATOM | 1821 | ND1 | HIS | 244 | 17.405 | 16.132 | 48.511 | 1.00 | 36.48 | B |
| ATOM | 1822 | CE1 | HIS | 244 | 17.080 | 16.392 | 49.765 | 1.00 | 35.67 | B |
| ATOM | 1823 | NE2 | HIS | 244 | 15.827 | 16.018 | 49.958 | 1.00 | 35.06 | B |
| ATOM | 1824 | C | HIS | 244 | 16.700 | 13.383 | 44.693 | 1.00 | 33.70 | B |
| ATOM | 1825 | O | HIS | 244 | 17.271 | 14.020 | 43.798 | 1.00 | 33.29 | B |
| ATOM | 1826 | N | MET | 245 | 15.885 | 12.366 | 44.448 | 1.00 | 34.30 | B |
| ATOM | 1827 | CA | MET | 245 | 15.654 | 11.910 | 43.087 | 1.00 | 34.70 | B |
| ATOM | 1828 | CB | MET | 245 | 16.212 | 10.483 | 42.944 | 1.00 | 34.85 | B |
| ATOM | 1829 | CG | MET | 245 | 17.734 | 10.441 | 43.100 | 1.00 | 35.80 | B |
| ATOM | 1830 | SD | MET | 245 | 18.439 | 8.805 | 43.321 | 1.00 | 36.13 | B |
| ATOM | 1831 | CE | MET | 245 | 18.009 | 8.537 | 45.032 | 1.00 | 32.87 | B |
| ATOM | 1832 | C | MET | 245 | 14.203 | 11.985 | 42.628 | 1.00 | 34.49 | B |
| ATOM | 1833 | O | MET | 245 | 13.272 | 11.757 | 43.402 | 1.00 | 33.49 | B |
| ATOM | 1834 | N | LYS | 246 | 14.026 | 12.313 | 41.352 | 1.00 | 35.05 | B |
| ATOM | 1835 | CA | LYS | 246 | 12.700 | 12.449 | 40.769 | 1.00 | 36.99 | B |
| ATOM | 1836 | CB | LYS | 246 | 12.280 | 13.947 | 40.750 | 1.00 | 38.69 | B |
| ATOM | 1837 | CG | LYS | 246 | 10.919 | 14.227 | 40.117 | 1.00 | 43.46 | B |
| ATOM | 1838 | CD | LYS | 246 | 10.702 | 15.729 | 39.856 | 1.00 | 45.60 | B |
| ATOM | 1839 | CE | LYS | 246 | 10.795 | 16.556 | 41.148 | 1.00 | 48.45 | B |
| ATOM | 1840 | NZ | LYS | 246 | 10.619 | 18.031 | 40.940 | 1.00 | 46.59 | B |
| ATOM | 1841 | C | LYS | 246 | 12.654 | 11.889 | 39.353 | 1.00 | 36.70 | B |
| ATOM | 1842 | O | LYS | 246 | 13.324 | 12.387 | 38.452 | 1.00 | 36.63 | B |
| ATOM | 1843 | N | GLU | 247 | 11.864 | 10.841 | 39.166 | 1.00 | 36.80 | B |
| ATOM | 1844 | CA | GLU | 247 | 11.706 | 10.240 | 37.854 | 1.00 | 37.12 | B |
| ATOM | 1845 | CB | GLU | 247 | 12.209 | 8.806 | 37.866 | 1.00 | 37.24 | B |
| ATOM | 1846 | CG | GLU | 247 | 11.710 | 7.990 | 39.036 | 1.00 | 37.73 | B |
| ATOM | 1847 | CD | GLU | 247 | 12.621 | 6.820 | 39.347 | 1.00 | 38.20 | B |
| ATOM | 1848 | OE1 | GLU | 247 | 12.293 | 6.035 | 40.262 | 1.00 | 37.07 | B |
| ATOM | 1849 | OE2 | GLU | 247 | 13.670 | 6.692 | 38.677 | 1.00 | 38.76 | B |
| ATOM | 1850 | C | GLU | 247 | 10.228 | 10.299 | 37.498 | 1.00 | 36.40 | B |
| ATOM | 1851 | O | GLU | 247 | 9.369 | 10.193 | 38.365 | 1.00 | 35.41 | B |
| ATOM | 1852 | N | THR | 248 | 9.940 | 10.498 | 36.219 | 1.00 | 37.67 | B |
| ATOM | 1853 | CA | THR | 248 | 8.563 | 10.587 | 35.746 | 1.00 | 39.02 | B |
| ATOM | 1854 | CB | THR | 248 | 8.344 | 11.889 | 34.920 | 1.00 | 39.40 | B |
| ATOM | 1855 | OG1 | THR | 248 | 8.754 | 13.025 | 35.693 | 1.00 | 40.65 | B |
| ATOM | 1856 | CG2 | THR | 248 | 6.877 | 12.050 | 34.543 | 1.00 | 40.08 | B |
| ATOM | 1857 | C | THR | 248 | 8.240 | 9.381 | 34.863 | 1.00 | 39.45 | B |
| ATOM | 1858 | O | THR | 248 | 8.959 | 9.095 | 33.902 | 1.00 | 39.20 | B |
| ATOM | 1859 | N | THR | 249 | 7.158 | 8.678 | 35.187 | 1.00 | 39.85 | B |
| ATOM | 1860 | CA | THR | 249 | 6.751 | 7.515 | 34.407 | 1.00 | 40.93 | B |
| ATOM | 1861 | CB | THR | 249 | 5.642 | 6.728 | 35.119 | 1.00 | 41.31 | B |
| ATOM | 1862 | OG1 | THR | 249 | 4.458 | 7.531 | 35.190 | 1.00 | 40.33 | B |
| ATOM | 1863 | CG2 | THR | 249 | 6.078 | 6.345 | 36.527 | 1.00 | 39.92 | B |
| ATOM | 1864 | C | THR | 249 | 6.233 | 7.952 | 33.039 | 1.00 | 41.94 | B |
| ATOM | 1865 | O | THR | 249 | 6.178 | 9.145 | 32.736 | 1.00 | 41.92 | B |
| ATOM | 1866 | N | ILE | 250 | 5.857 | 6.979 | 32.214 | 1.00 | 43.64 | B |
| ATOM | 1867 | CA | ILE | 250 | 5.343 | 7.253 | 30.875 | 1.00 | 43.57 | B |
| ATOM | 1868 | CB | ILE | 250 | 5.340 | 5.970 | 30.004 | 1.00 | 43.38 | B |
| ATOM | 1869 | CG2 | ILE | 250 | 4.228 | 5.029 | 30.465 | 1.00 | 41.86 | B |
| ATOM | 1870 | CG1 | ILE | 250 | 5.173 | 6.343 | 28.510 | 1.00 | 41.89 | B |
| ATOM | 1871 | CD1 | ILE | 250 | 5.286 | 5.169 | 27.560 | 1.00 | 39.31 | B |
| ATOM | 1872 | C | ILE | 250 | 3.922 | 7.805 | 30.983 | 1.00 | 44.06 | B |
| ATOM | 1873 | O | ILE | 250 | 3.320 | 8.197 | 29.984 | 1.00 | 43.16 | B |
| ATOM | 1874 | N | ASP | 251 | 3.402 | 7.834 | 32.209 | 1.00 | 45.37 | B |
| ATOM | 1875 | CA | ASP | 251 | 2.059 | 8.353 | 32.493 | 1.00 | 47.36 | B |
| ATOM | 1876 | CB | ASP | 251 | 1.319 | 7.437 | 33.502 | 1.00 | 47.52 | B |
| ATOM | 1877 | CG | ASP | 251 | 0.719 | 6.208 | 32.852 | 1.00 | 46.95 | B |
| ATOM | 1878 | OD1 | ASP | 251 | 0.222 | 5.335 | 33.595 | 1.00 | 46.42 | B |
| ATOM | 1879 | OD2 | ASP | 251 | 0.735 | 6.121 | 31.606 | 1.00 | 46.77 | B |
| ATOM | 1880 | C | ASP | 251 | 2.097 | 9.778 | 33.061 | 1.00 | 48.00 | B |
| ATOM | 1881 | O | ASP | 251 | 1.052 | 10.349 | 33.377 | 1.00 | 49.62 | B |
| ATOM | 1882 | N | GLY | 252 | 3.297 | 10.339 | 33.195 | 1.00 | 48.57 | B |
| ATOM | 1883 | CA | GLY | 252 | 3.445 | 11.684 | 33.725 | 1.00 | 48.41 | B |
| ATOM | 1884 | C | GLY | 252 | 3.519 | 11.749 | 35.243 | 1.00 | 49.25 | B |
| ATOM | 1885 | O | GLY | 252 | 3.592 | 12.839 | 35.823 | 1.00 | 48.30 | B |
| ATOM | 1886 | N | GLU | 253 | 3.489 | 10.584 | 35.890 | 1.00 | 49.52 | B |
| ATOM | 1887 | CA | GLU | 253 | 3.555 | 10.504 | 37.349 | 1.00 | 49.94 | B |
| ATOM | 1888 | CB | GLU | 253 | 2.989 | 9.156 | 37.839 | 1.00 | 51.87 | B |
| ATOM | 1889 | CG | GLU | 253 | 3.083 | 8.942 | 39.349 | 1.00 | 55.20 | B |
| ATOM | 1890 | CD | GLU | 253 | 2.805 | 7.498 | 39.764 | 1.00 | 57.60 | B |
| ATOM | 1891 | OE1 | GLU | 253 | 2.837 | 7.204 | 40.981 | 1.00 | 58.27 | B |
| ATOM | 1892 | OE2 | GLU | 253 | 2.558 | 6.655 | 38.875 | 1.00 | 58.42 | B |
| ATOM | 1893 | C | GLU | 253 | 4.996 | 10.659 | 37.835 | 1.00 | 49.08 | B |
| ATOM | 1894 | O | GLU | 253 | 5.948 | 10.301 | 37.136 | 1.00 | 47.88 | B |
| ATOM | 1895 | N | GLU | 254 | 5.148 | 11.187 | 39.043 | 1.00 | 48.18 | B |
| ATOM | 1896 | CA | GLU | 254 | 6.471 | 11.394 | 39.610 | 1.00 | 48.03 | B |
| ATOM | 1897 | CB | GLU | 254 | 6.633 | 12.854 | 40.000 | 1.00 | 48.74 | B |
| ATOM | 1898 | CG | GLU | 254 | 6.950 | 13.761 | 38.827 | 1.00 | 51.39 | B |
| ATOM | 1899 | CD | GLU | 254 | 6.866 | 15.232 | 39.193 | 1.00 | 53.81 | B |
| ATOM | 1900 | OE1 | GLU | 254 | 7.184 | 15.575 | 40.356 | 1.00 | 54.50 | B |
| ATOM | 1901 | OE2 | GLU | 254 | 6.493 | 16.043 | 38.313 | 1.00 | 54.20 | B |
| ATOM | 1902 | C | GLU | 254 | 6.817 | 10.497 | 40.797 | 1.00 | 46.73 | B |
| ATOM | 1903 | O | GLU | 254 | 6.111 | 10.466 | 41.805 | 1.00 | 46.07 | B |
| ATOM | 1904 | N | LEU | 255 | 7.918 | 9.763 | 40.651 | 1.00 | 45.44 | B |
| ATOM | 1905 | CA | LEU | 255 | 8.416 | 8.869 | 41.689 | 1.00 | 43.34 | B |
| ATOM | 1906 | CB | LEU | 255 | 8.880 | 7.522 | 41.069 | 1.00 | 42.70 | B |
| ATOM | 1907 | CG | LEU | 255 | 7.888 | 6.755 | 40.138 | 1.00 | 42.10 | B |
| ATOM | 1908 | CD1 | LEU | 255 | 8.584 | 5.548 | 39.528 | 1.00 | 41.93 | B |
| ATOM | 1909 | CD2 | LEU | 255 | 6.658 | 6.322 | 40.919 | 1.00 | 42.42 | B |
| ATOM | 1910 | C | LEU | 255 | 9.603 | 9.591 | 42.329 | 1.00 | 42.63 | B |
| ATOM | 1911 | O | LEU | 255 | 10.599 | 9.886 | 41.662 | 1.00 | 40.70 | B |
| ATOM | 1912 | N | VAL | 256 | 9.484 | 9.890 | 43.617 | 1.00 | 41.65 | B |
| ATOM | 1913 | CA | VAL | 256 | 10.540 | 10.594 | 44.326 | 1.00 | 41.53 | B |
| ATOM | 1914 | CB | VAL | 256 | 9.994 | 11.865 | 45.040 | 1.00 | 42.73 | B |
| ATOM | 1915 | CG1 | VAL | 256 | 9.445 | 12.851 | 44.013 | 1.00 | 41.79 | B |
| ATOM | 1916 | CG2 | VAL | 256 | 8.899 | 11.487 | 46.028 | 1.00 | 43.14 | B |
| ATOM | 1917 | C | VAL | 256 | 11.192 | 9.691 | 45.357 | 1.00 | 40.91 | B |
| ATOM | 1918 | O | VAL | 256 | 10.516 | 9.123 | 46.216 | 1.00 | 42.52 | B |
| ATOM | 1919 | N | LYS | 257 | 12.507 | 9.542 | 45.255 | 1.00 | 38.10 | B |
| ATOM | 1920 | CA | LYS | 257 | 13.237 | 8.718 | 46.200 | 1.00 | 35.97 | B |
| ATOM | 1921 | CB | LYS | 257 | 13.712 | 7.370 | 45.525 | 1.00 | 37.07 | B |
| ATOM | 1922 | CG | LYS | 257 | 14.482 | 7.490 | 44.219 | 1.00 | 35.97 | B |
| ATOM | 1923 | CD | LYS | 257 | 14.612 | 6.108 | 43.592 | 1.00 | 34.96 | B |
| ATOM | 1924 | CE | LYS | 257 | 15.566 | 6.085 | 42.412 | 1.00 | 36.06 | B |
| ATOM | 1925 | NZ | LYS | 257 | 15.142 | 6.972 | 41.303 | 1.00 | 38.19 | B |
| ATOM | 1926 | C | LYS | 257 | 14.408 | 9.497 | 46.777 | 1.00 | 34.33 | B |
| ATOM | 1927 | O | LYS | 257 | 15.100 | 10.227 | 46.074 | 1.00 | 35.94 | B |
| ATOM | 1928 | N | ILE | 258 | 14.618 | 9.345 | 48.074 | 1.00 | 31.24 | B |
| ATOM | 1929 | CA | ILE | 258 | 15.677 | 10.066 | 48.747 | 1.00 | 27.10 | B |
| ATOM | 1930 | CB | ILE | 258 | 15.077 | 10.988 | 49.842 | 1.00 | 28.34 | B |
| ATOM | 1931 | CG2 | ILE | 258 | 16.181 | 11.791 | 50.516 | 1.00 | 26.47 | B |
| ATOM | 1932 | CG1 | ILE | 258 | 14.021 | 11.949 | 49.203 | 1.00 | 27.71 | B |
| ATOM | 1933 | CD1 | ILE | 258 | 13.168 | 12.703 | 50.214 | 1.00 | 25.91 | B |
| ATOM | 1934 | C | ILE | 258 | 16.695 | 9.136 | 49.382 | 1.00 | 24.38 | B |
| ATOM | 1935 | O | ILE | 258 | 16.386 | 8.400 | 50.314 | 1.00 | 22.26 | B |
| ATOM | 1936 | N | GLY | 259 | 17.917 | 9.182 | 48.872 | 1.00 | 22.97 | B |
| ATOM | 1937 | CA | GLY | 259 | 18.975 | 8.359 | 49.422 | 1.00 | 22.93 | B |
| ATOM | 1938 | C | GLY | 259 | 20.055 | 9.163 | 50.135 | 1.00 | 22.70 | B |
| ATOM | 1939 | O | GLY | 259 | 20.561 | 10.161 | 49.609 | 1.00 | 21.85 | B |
| ATOM | 1940 | N | LYS | 260 | 20.410 | 8.731 | 51.339 | 1.00 | 21.39 | B |
| ATOM | 1941 | CA | LYS | 260 | 21.441 | 9.412 | 52.112 | 1.00 | 21.77 | B |
| ATOM | 1942 | CB | LYS | 260 | 20.834 | 10.042 | 53.411 | 1.00 | 20.00 | B |
| ATOM | 1943 | CG | LYS | 260 | 21.805 | 10.848 | 54.262 | 1.00 | 17.18 | B |
| ATOM | 1944 | CD | LYS | 260 | 21.119 | 11.342 | 55.534 | 1.00 | 16.09 | B |
| ATOM | 1945 | CE | LYS | 260 | 22.049 | 12.181 | 56.417 | 1.00 | 16.97 | B |
| ATOM | 1946 | NZ | LYS | 260 | 21.341 | 12.724 | 57.641 | 1.00 | 15.85 | B |
| ATOM | 1947 | C | LYS | 260 | 22.545 | 8.419 | 52.469 | 1.00 | 21.92 | B |
| ATOM | 1948 | O | LYS | 260 | 22.284 | 7.303 | 52.938 | 1.00 | 22.32 | B |
| ATOM | 1949 | N | LEU | 261 | 23.780 | 8.837 | 52.236 | 1.00 | 19.52 | B |
| ATOM | 1950 | CA | LEU | 261 | 24.932 | 8.009 | 52.520 | 1.00 | 17.05 | B |
| ATOM | 1951 | CB | LEU | 261 | 25.693 | 7.741 | 51.235 | 1.00 | 14.85 | B |
| ATOM | 1952 | CG | LEU | 261 | 27.111 | 7.236 | 51.385 | 1.00 | 14.96 | B |
| ATOM | 1953 | CD1 | LEU | 261 | 27.114 | 5.939 | 52.165 | 1.00 | 12.47 | B |
| ATOM | 1954 | CD2 | LEU | 261 | 27.730 | 7.054 | 50.019 | 1.00 | 12.11 | B |
| ATOM | 1955 | C | LEU | 261 | 25.828 | 8.720 | 53.519 | 1.00 | 17.96 | B |
| ATOM | 1956 | O | LEU | 261 | 26.258 | 9.850 | 53.284 | 1.00 | 16.25 | B |
| ATOM | 1957 | N | ASN | 262 | 26.099 | 8.063 | 54.643 | 1.00 | 18.12 | B |
| ATOM | 1958 | CA | ASN | 262 | 26.970 | 8.640 | 55.670 | 1.00 | 18.04 | B |
| ATOM | 1959 | CB | ASN | 262 | 26.336 | 8.512 | 57.080 | 1.00 | 15.45 | B |
| ATOM | 1960 | CG | ASN | 262 | 24.943 | 9.103 | 57.152 | 1.00 | 17.34 | B |
| ATOM | 1961 | OD1 | ASN | 262 | 23.957 | 8.381 | 57.282 | 1.00 | 17.52 | B |
| ATOM | 1962 | ND2 | ASN | 262 | 24.855 | 10.420 | 57.070 | 1.00 | 17.02 | B |
| ATOM | 1963 | C | ASN | 262 | 28.327 | 7.929 | 55.664 | 1.00 | 18.26 | B |
| ATOM | 1964 | O | ASN | 262 | 28.399 | 6.697 | 55.735 | 1.00 | 16.87 | B |
| ATOM | 1965 | N | LEU | 263 | 29.394 | 8.717 | 55.564 | 1.00 | 18.04 | B |
| ATOM | 1966 | CA | LEU | 263 | 30.759 | 8.200 | 55.560 | 1.00 | 17.90 | B |
| ATOM | 1967 | CB | LEU | 263 | 31.482 | 8.723 | 54.339 | 1.00 | 15.70 | B |
| ATOM | 1968 | CG | LEU | 263 | 30.717 | 8.283 | 53.075 | 1.00 | 17.05 | B |
| ATOM | 1969 | CD1 | LEU | 263 | 31.255 | 8.961 | 51.853 | 1.00 | 16.38 | B |
| ATOM | 1970 | CD2 | LEU | 263 | 30.812 | 6.754 | 52.929 | 1.00 | 18.46 | B |
| ATOM | 1971 | C | LEU | 263 | 31.411 | 8.688 | 56.849 | 1.00 | 18.79 | B |
| ATOM | 1972 | O | LEU | 263 | 31.712 | 9.873 | 56.992 | 1.00 | 20.38 | B |
| ATOM | 1973 | N | VAL | 264 | 31.614 | 7.774 | 57.794 | 1.00 | 18.49 | B |
| ATOM | 1974 | CA | VAL | 264 | 32.183 | 8.128 | 59.093 | 1.00 | 18.30 | B |
| ATOM | 1975 | CB | VAL | 264 | 31.335 | 7.529 | 60.228 | 1.00 | 18.68 | B |
| ATOM | 1976 | CG1 | VAL | 264 | 31.752 | 8.115 | 61.561 | 1.00 | 17.56 | B |
| ATOM | 1977 | CG2 | VAL | 264 | 29.858 | 7.772 | 59.955 | 1.00 | 21.14 | B |
| ATOM | 1978 | C | VAL | 264 | 33.627 | 7.696 | 59.333 | 1.00 | 19.31 | B |
| ATOM | 1979 | O | VAL | 264 | 33.952 | 6.513 | 59.210 | 1.00 | 19.80 | B |
| ATOM | 1980 | N | ASP | 265 | 34.478 | 8.667 | 59.680 | 1.00 | 17.61 | B |
| ATOM | 1981 | CA | ASP | 265 | 35.880 | 8.419 | 59.995 | 1.00 | 15.36 | B |
| ATOM | 1982 | CB | ASP | 265 | 36.771 | 9.484 | 59.355 | 1.00 | 14.42 | B |
| ATOM | 1983 | CG | ASP | 265 | 38.258 | 9.279 | 59.658 | 1.00 | 16.29 | B |
| ATOM | 1984 | OD1 | ASP | 265 | 38.583 | 8.741 | 60.736 | 1.00 | 19.48 | B |
| ATOM | 1985 | OD2 | ASP | 265 | 39.110 | 9.677 | 58.832 | 1.00 | 16.17 | B |
| ATOM | 1986 | C | ASP | 265 | 35.971 | 8.507 | 61.528 | 1.00 | 15.62 | B |
| ATOM | 1987 | O | ASP | 265 | 36.119 | 9.593 | 62.086 | 1.00 | 17.19 | B |
| ATOM | 1988 | N | LEU | 266 | 35.891 | 7.367 | 62.205 | 1.00 | 13.53 | B |
| ATOM | 1989 | CA | LEU | 266 | 35.930 | 7.357 | 63.666 | 1.00 | 12.99 | B |
| ATOM | 1990 | CB | LEU | 266 | 35.555 | 5.913 | 64.239 | 1.00 | 9.90 | B |
| ATOM | 1991 | CG | LEU | 266 | 34.172 | 5.339 | 63.898 | 1.00 | 12.88 | B |
| ATOM | 1992 | CD1 | LEU | 266 | 34.070 | 3.881 | 64.374 | 1.00 | 12.44 | B |
| ATOM | 1993 | CD2 | LEU | 266 | 33.088 | 6.185 | 64.542 | 1.00 | 11.19 | B |
| ATOM | 1994 | C | LEU | 266 | 37.277 | 7.783 | 64.240 | 1.00 | 11.25 | B |
| ATOM | 1995 | O | LEU | 266 | 38.274 | 7.867 | 63.532 | 1.00 | 7.77 | B |
| ATOM | 1996 | N | ALA | 267 | 37.263 | 8.059 | 65.539 | 1.00 | 10.58 | B |
| ATOM | 1997 | CA | ALA | 267 | 38.453 | 8.422 | 66.284 | 1.00 | 13.04 | B |
| ATOM | 1998 | CB | ALA | 267 | 38.057 | 9.029 | 67.634 | 1.00 | 11.27 | B |
| ATOM | 1999 | C | ALA | 267 | 39.221 | 7.125 | 66.507 | 1.00 | 14.13 | B |
| ATOM | 2000 | O | ALA | 267 | 38.610 | 6.077 | 66.718 | 1.00 | 16.34 | B |
| ATOM | 2001 | N | GLY | 268 | 40.546 | 7.190 | 66.475 | 1.00 | 14.85 | B |
| ATOM | 2002 | CA | GLY | 268 | 41.347 | 5.999 | 66.688 | 1.00 | 17.83 | B |
| ATOM | 2003 | C | GLY | 268 | 40.934 | 5.198 | 67.909 | 1.00 | 20.15 | B |
| ATOM | 2004 | O | GLY | 268 | 40.663 | 5.760 | 68.978 | 1.00 | 21.52 | B |
| ATOM | 2005 | N | SER | 269 | 40.918 | 3.878 | 67.773 | 1.00 | 20.60 | B |
| ATOM | 2006 | CA | SER | 269 | 40.500 | 3.017 | 68.878 | 1.00 | 23.05 | B |
| ATOM | 2007 | CB | SER | 269 | 39.929 | 1.721 | 68.324 | 1.00 | 20.23 | B |
| ATOM | 2008 | OG | SER | 269 | 40.842 | 1.099 | 67.442 | 1.00 | 17.43 | B |
| ATOM | 2009 | C | SER | 269 | 41.546 | 2.678 | 69.941 | 1.00 | 26.49 | B |
| ATOM | 2010 | O | SER | 269 | 41.227 | 1.969 | 70.903 | 1.00 | 27.04 | B |
| ATOM | 2011 | N | GLU | 270 | 42.775 | 3.171 | 69.781 | 1.00 | 29.47 | B |
| ATOM | 2012 | CA | GLU | 270 | 43.848 | 2.887 | 70.743 | 1.00 | 32.95 | B |
| ATOM | 2013 | CB | GLU | 270 | 45.234 | 3.432 | 70.210 | 1.00 | 32.65 | B |
| ATOM | 2014 | CG | GLU | 270 | 45.405 | 4.968 | 70.193 | 1.00 | 30.27 | B |
| ATOM | 2015 | CD | GLU | 270 | 44.822 | 5.656 | 68.963 | 1.00 | 30.89 | B |
| ATOM | 2016 | OE1 | GLU | 270 | 44.879 | 6.908 | 68.911 | 1.00 | 32.19 | B |
| ATOM | 2017 | OE2 | GLU | 270 | 44.315 | 4.961 | 68.052 | 1.00 | 28.80 | B |
| ATOM | 2018 | C | GLU | 270 | 43.560 | 3.472 | 72.129 | 1.00 | 36.87 | B |
| ATOM | 2019 | O | GLU | 270 | 43.380 | 4.681 | 72.277 | 1.00 | 39.21 | B |
| ATOM | 2020 | N | ASN | 271 | 43.503 | 2.613 | 73.143 | 1.00 | 40.27 | B |
| ATOM | 2021 | CA | ASN | 271 | 43.238 | 3.062 | 74.515 | 1.00 | 42.68 | B |
| ATOM | 2022 | CB | ASN | 271 | 42.196 | 2.131 | 75.222 | 1.00 | 43.15 | B |
| ATOM | 2023 | CG | ASN | 271 | 40.798 | 2.244 | 74.621 | 1.00 | 45.39 | B |
| ATOM | 2024 | OD1 | ASN | 271 | 40.230 | 3.337 | 74.540 | 1.00 | 46.39 | B |
| ATOM | 2025 | ND2 | ASN | 271 | 40.232 | 1.109 | 74.210 | 1.00 | 43.39 | B |
| ATOM | 2026 | C | ASN | 271 | 44.528 | 3.093 | 75.331 | 1.00 | 43.55 | B |
| ATOM | 2027 | O | ASN | 271 | 45.603 | 2.746 | 74.833 | 1.00 | 43.93 | B |
| ATOM | 2028 | N | ASN | 287 | 41.588 | 11.864 | 79.666 | 1.00 | 44.94 | B |
| ATOM | 2029 | CA | ASN | 287 | 40.716 | 12.252 | 78.558 | 1.00 | 45.22 | B |
| ATOM | 2030 | CB | ASN | 287 | 41.514 | 13.086 | 77.476 | 1.00 | 48.29 | B |
| ATOM | 2031 | CG | ASN | 287 | 42.261 | 14.276 | 78.074 | 1.00 | 50.68 | B |
| ATOM | 2032 | OD1 | ASN | 287 | 43.249 | 14.106 | 78.796 | 1.00 | 51.76 | B |
| ATOM | 2033 | ND2 | ASN | 287 | 41.791 | 15.488 | 77.774 | 1.00 | 51.75 | B |
| ATOM | 2034 | C | ASN | 287 | 40.091 | 11.016 | 77.897 | 1.00 | 42.90 | B |
| ATOM | 2035 | O | ASN | 287 | 40.787 | 10.182 | 77.315 | 1.00 | 42.06 | B |
| ATOM | 2036 | N | ILE | 288 | 38.771 | 10.914 | 77.995 | 1.00 | 40.12 | B |
| ATOM | 2037 | CA | ILE | 288 | 38.034 | 9.794 | 77.424 | 1.00 | 36.62 | B |
| ATOM | 2038 | CB | ILE | 288 | 37.110 | 9.146 | 78.479 | 1.00 | 37.65 | B |
| ATOM | 2039 | CG2 | ILE | 288 | 37.911 | 8.154 | 79.325 | 1.00 | 38.70 | B |
| ATOM | 2040 | CG1 | ILE | 288 | 36.464 | 10.252 | 79.390 | 1.00 | 36.64 | B |
| ATOM | 2041 | CD1 | ILE | 288 | 35.583 | 11.252 | 78.657 | 1.00 | 36.28 | B |
| ATOM | 2042 | C | ILE | 288 | 37.183 | 10.200 | 76.230 | 1.00 | 33.35 | B |
| ATOM | 2043 | O | ILE | 288 | 36.763 | 11.356 | 76.100 | 1.00 | 34.53 | B |
| ATOM | 2044 | N | ASN | 289 | 36.938 | 9.252 | 75.342 | 1.00 | 27.16 | B |
| ATOM | 2045 | CA | ASN | 289 | 36.112 | 9.564 | 74.199 | 1.00 | 23.25 | B |
| ATOM | 2046 | CB | ASN | 289 | 36.731 | 9.052 | 72.954 | 1.00 | 20.82 | B |
| ATOM | 2047 | CG | ASN | 289 | 36.172 | 9.721 | 71.712 | 1.00 | 19.85 | B |
| ATOM | 2048 | OD1 | ASN | 289 | 36.929 | 10.208 | 70.878 | 1.00 | 19.66 | B |
| ATOM | 2049 | ND2 | ASN | 289 | 34.846 | 9.737 | 71.576 | 1.00 | 17.37 | B |
| ATOM | 2050 | C | ASN | 289 | 34.763 | 8.912 | 74.459 | 1.00 | 20.79 | B |
| ATOM | 2051 | O | ASN | 289 | 34.553 | 7.735 | 74.170 | 1.00 | 18.65 | B |
| ATOM | 2052 | N | GLN | 290 | 33.863 | 9.694 | 75.042 | 1.00 | 19.57 | B |
| ATOM | 2053 | CA | GLN | 290 | 32.537 | 9.216 | 75.379 | 1.00 | 19.29 | B |
| ATOM | 2054 | CB | GLN | 290 | 31.678 | 10.366 | 75.901 | 1.00 | 19.26 | B |
| ATOM | 2055 | CG | GLN | 290 | 30.278 | 9.942 | 76.312 | 1.00 | 19.65 | B |
| ATOM | 2056 | CD | GLN | 290 | 30.265 | 8.891 | 77.423 | 1.00 | 20.79 | B |
| ATOM | 2057 | OE1 | GLN | 290 | 29.211 | 8.339 | 77.754 | 1.00 | 21.88 | B |
| ATOM | 2058 | NE2 | GLN | 290 | 31.427 | 8.621 | 78.006 | 1.00 | 18.18 | B |
| ATOM | 2059 | C | GLN | 290 | 31.830 | 8.538 | 74.214 | 1.00 | 18.80 | B |
| ATOM | 2060 | O | GLN | 290 | 31.199 | 7.502 | 74.397 | 1.00 | 17.47 | B |
| ATOM | 2061 | N | SER | 291 | 31.939 | 9.122 | 73.021 | 1.00 | 18.97 | B |
| ATOM | 2062 | CA | SER | 291 | 31.289 | 8.565 | 71.841 | 1.00 | 18.84 | B |
| ATOM | 2063 | CB | SER | 291 | 31.326 | 9.565 | 70.646 | 1.00 | 19.15 | B |
| ATOM | 2064 | OG | SER | 291 | 30.347 | 10.593 | 70.784 | 1.00 | 19.00 | B |
| ATOM | 2065 | C | SER | 291 | 31.897 | 7.239 | 71.420 | 1.00 | 19.68 | B |
| ATOM | 2066 | O | SER | 291 | 31.173 | 6.323 | 71.027 | 1.00 | 21.26 | B |
| ATOM | 2067 | N | LEU | 292 | 33.219 | 7.131 | 71.494 | 1.00 | 18.43 | B |
| ATOM | 2068 | CA | LEU | 292 | 33.872 | 5.888 | 71.128 | 1.00 | 17.73 | B |
| ATOM | 2069 | CB | LEU | 292 | 35.361 | 6.070 | 71.140 | 1.00 | 15.77 | B |
| ATOM | 2070 | CG | LEU | 292 | 36.119 | 4.969 | 70.418 | 1.00 | 15.31 | B |
| ATOM | 2071 | CD1 | LEU | 292 | 35.703 | 4.951 | 68.953 | 1.00 | 11.07 | B |
| ATOM | 2072 | CD2 | LEU | 292 | 37.621 | 5.213 | 70.548 | 1.00 | 16.30 | B |
| ATOM | 2073 | C | LEU | 292 | 33.461 | 4.827 | 72.159 | 1.00 | 19.37 | B |
| ATOM | 2074 | O | LEU | 292 | 33.107 | 3.698 | 71.814 | 1.00 | 20.03 | B |
| ATOM | 2075 | N | LEU | 293 | 33.504 | 5.219 | 73.430 | 1.00 | 19.01 | B |
| ATOM | 2076 | CA | LEU | 293 | 33.137 | 4.357 | 74.531 | 1.00 | 18.18 | B |
| ATOM | 2077 | CB | LEU | 293 | 33.194 | 5.140 | 75.819 | 1.00 | 16.50 | B |
| ATOM | 2078 | CG | LEU | 293 | 34.193 | 4.752 | 76.903 | 1.00 | 18.80 | B |
| ATOM | 2079 | CD1 | LEU | 293 | 35.291 | 3.824 | 76.354 | 1.00 | 14.59 | B |
| ATOM | 2080 | CD2 | LEU | 293 | 34.789 | 6.039 | 77.485 | 1.00 | 18.33 | B |
| ATOM | 2081 | C | LEU | 293 | 31.724 | 3.828 | 74.326 | 1.00 | 20.79 | B |
| ATOM | 2082 | O | LEU | 293 | 31.446 | 2.629 | 74.480 | 1.00 | 21.79 | B |
| ATOM | 2083 | N | THR | 294 | 30.824 | 4.730 | 73.972 | 1.00 | 20.82 | B |
| ATOM | 2084 | CA | THR | 294 | 29.444 | 4.348 | 73.785 | 1.00 | 21.70 | B |
| ATOM | 2085 | CB | THR | 294 | 28.556 | 5.607 | 73.770 | 1.00 | 21.45 | B |
| ATOM | 2086 | OG1 | THR | 294 | 28.737 | 6.305 | 75.012 | 1.00 | 20.05 | B |
| ATOM | 2087 | CG2 | THR | 294 | 27.085 | 5.243 | 73.638 | 1.00 | 23.08 | B |
| ATOM | 2088 | C | THR | 294 | 29.245 | 3.488 | 72.541 | 1.00 | 22.57 | B |
| ATOM | 2089 | O | THR | 294 | 28.410 | 2.589 | 72.541 | 1.00 | 24.83 | B |
| ATOM | 2090 | N | LEU | 295 | 30.028 | 3.726 | 71.492 | 1.00 | 22.48 | B |
| ATOM | 2091 | CA | LEU | 295 | 29.888 | 2.929 | 70.278 | 1.00 | 20.67 | B |
| ATOM | 2092 | CB | LEU | 295 | 30.896 | 3.354 | 69.239 | 1.00 | 16.50 | B |
| ATOM | 2093 | CG | LEU | 295 | 30.872 | 2.542 | 67.933 | 1.00 | 15.31 | B |
| ATOM | 2094 | CD1 | LEU | 295 | 29.480 | 2.540 | 67.301 | 1.00 | 9.83 | B |
| ATOM | 2095 | CD2 | LEU | 295 | 31.901 | 3.126 | 66.996 | 1.00 | 13.69 | B |
| ATOM | 2096 | C | LEU | 295 | 30.072 | 1.453 | 70.614 | 1.00 | 21.75 | B |
| ATOM | 2097 | O | LEU | 295 | 29.261 | 0.620 | 70.222 | 1.00 | 22.82 | B |
| ATOM | 2098 | N | GLY | 296 | 31.141 | 1.141 | 71.345 | 1.00 | 22.87 | B |
| ATOM | 2099 | CA | GLY | 296 | 31.402 | −0.230 | 71.753 | 1.00 | 21.35 | B |
| ATOM | 2100 | C | GLY | 296 | 30.318 | −0.785 | 72.668 | 1.00 | 20.58 | B |
| ATOM | 2101 | O | GLY | 296 | 29.960 | −1.950 | 72.566 | 1.00 | 22.84 | B |
| ATOM | 2102 | N | ARG | 297 | 29.782 | 0.034 | 73.562 | 1.00 | 19.00 | B |
| ATOM | 2103 | CA | ARG | 297 | 28.735 | −0.441 | 74.462 | 1.00 | 18.91 | B |
| ATOM | 2104 | CB | ARG | 297 | 28.530 | 0.539 | 75.601 | 1.00 | 17.91 | B |
| ATOM | 2105 | CG | ARG | 297 | 29.645 | 0.523 | 76.596 | 1.00 | 17.55 | B |
| ATOM | 2106 | CD | ARG | 297 | 29.622 | 1.775 | 77.433 | 1.00 | 21.12 | B |
| ATOM | 2107 | NE | ARG | 297 | 30.783 | 1.860 | 78.311 | 1.00 | 20.84 | B |
| ATOM | 2108 | CZ | ARG | 297 | 31.212 | 2.987 | 78.862 | 1.00 | 19.95 | B |
| ATOM | 2109 | NH1 | ARG | 297 | 30.567 | 4.118 | 78.614 | 1.00 | 19.89 | B |
| ATOM | 2110 | NH2 | ARG | 297 | 32.274 | 2.982 | 79.661 | 1.00 | 15.55 | B |
| ATOM | 2111 | C | ARG | 297 | 27.419 | −0.662 | 73.733 | 1.00 | 18.05 | B |
| ATOM | 2112 | O | ARG | 297 | 26.581 | −1.440 | 74.177 | 1.00 | 18.18 | B |
| ATOM | 2113 | N | VAL | 298 | 27.235 | 0.035 | 72.618 | 1.00 | 19.06 | B |
| ATOM | 2114 | CA | VAL | 298 | 26.019 | −0.106 | 71.823 | 1.00 | 17.97 | B |
| ATOM | 2115 | CB | VAL | 298 | 25.816 | 1.111 | 70.885 | 1.00 | 15.95 | B |
| ATOM | 2116 | CG1 | VAL | 298 | 24.691 | 0.843 | 69.899 | 1.00 | 13.08 | B |
| ATOM | 2117 | CG2 | VAL | 298 | 25.507 | 2.350 | 71.710 | 1.00 | 14.44 | B |
| ATOM | 2118 | C | VAL | 298 | 26.140 | −1.377 | 70.985 | 1.00 | 19.67 | B |
| ATOM | 2119 | O | VAL | 298 | 25.153 | −2.075 | 70.749 | 1.00 | 21.91 | B |
| ATOM | 2120 | N | ILE | 299 | 27.356 | −1.686 | 70.544 | 1.00 | 19.47 | B |
| ATOM | 2121 | CA | ILE | 299 | 27.570 | −2.879 | 69.736 | 1.00 | 21.25 | B |
| ATOM | 2122 | CB | ILE | 299 | 28.973 | −2.830 | 69.068 | 1.00 | 21.35 | B |
| ATOM | 2123 | CG2 | ILE | 299 | 29.354 | −4.192 | 68.502 | 1.00 | 19.14 | B |
| ATOM | 2124 | CG1 | ILE | 299 | 28.950 | −1.752 | 67.932 | 1.00 | 19.67 | B |
| ATOM | 2125 | CD1 | ILE | 299 | 30.316 | −1.238 | 67.523 | 1.00 | 19.64 | B |
| ATOM | 2126 | C | ILE | 299 | 27.399 | −4.122 | 70.610 | 1.00 | 22.50 | B |
| ATOM | 2127 | O | ILE | 299 | 26.774 | −5.102 | 70.206 | 1.00 | 21.52 | B |
| ATOM | 2128 | N | THR | 300 | 27.936 | −4.057 | 71.821 | 1.00 | 23.04 | B |
| ATOM | 2129 | CA | THR | 300 | 27.827 | −5.153 | 72.763 | 1.00 | 23.72 | B |
| ATOM | 2130 | CB | THR | 300 | 28.521 | −4.787 | 74.068 | 1.00 | 23.18 | B |
| ATOM | 2131 | OG1 | THR | 300 | 29.923 | −4.646 | 73.811 | 1.00 | 21.92 | B |
| ATOM | 2132 | CG2 | THR | 300 | 28.284 | −5.841 | 75.138 | 1.00 | 17.93 | B |
| ATOM | 2133 | C | THR | 300 | 26.353 | −5.447 | 73.020 | 1.00 | 27.59 | B |
| ATOM | 2134 | O | THR | 300 | 25.878 | −6.563 | 72.787 | 1.00 | 27.46 | B |
| ATOM | 2135 | N | ALA | 301 | 25.626 | −4.438 | 73.480 | 1.00 | 29.03 | B |
| ATOM | 2136 | CA | ALA | 301 | 24.206 | −4.600 | 73.754 | 1.00 | 30.76 | B |
| ATOM | 2137 | CB | ALA | 301 | 23.598 | −3.262 | 74.139 | 1.00 | 31.16 | B |
| ATOM | 2138 | C | ALA | 301 | 23.437 | −5.196 | 72.573 | 1.00 | 32.99 | B |
| ATOM | 2139 | O | ALA | 301 | 22.545 | −6.017 | 72.772 | 1.00 | 35.01 | B |
| ATOM | 2140 | N | LEU | 302 | 23.770 | −4.780 | 71.351 | 1.00 | 34.50 | B |
| ATOM | 2141 | CA | LEU | 302 | 23.088 | −5.279 | 70.152 | 1.00 | 34.70 | B |
| ATOM | 2142 | CB | LEU | 302 | 23.440 | −4.425 | 68.943 | 1.00 | 35.01 | B |
| ATOM | 2143 | CG | LEU | 302 | 22.840 | −2.999 | 68.895 | 1.00 | 35.55 | B |
| ATOM | 2144 | CD1 | LEU | 302 | 23.474 | −2.227 | 67.759 | 1.00 | 36.40 | B |
| ATOM | 2145 | CD2 | LEU | 302 | 21.334 | −3.063 | 68.714 | 1.00 | 33.89 | B |
| ATOM | 2146 | C | LEU | 302 | 23.451 | −6.721 | 69.855 | 1.00 | 35.87 | B |
| ATOM | 2147 | O | LEU | 302 | 22.590 | −7.547 | 69.549 | 1.00 | 36.50 | B |
| ATOM | 2148 | N | VAL | 303 | 24.742 | −7.008 | 69.941 | 1.00 | 36.97 | B |
| ATOM | 2149 | CA | VAL | 303 | 25.271 | −8.339 | 69.691 | 1.00 | 36.81 | B |
| ATOM | 2150 | CB | VAL | 303 | 26.818 | −8.289 | 69.707 | 1.00 | 36.26 | B |
| ATOM | 2151 | CG1 | VAL | 303 | 27.402 | −9.658 | 69.961 | 1.00 | 35.12 | B |
| ATOM | 2152 | CG2 | VAL | 303 | 27.316 | −7.726 | 68.384 | 1.00 | 35.06 | B |
| ATOM | 2153 | C | VAL | 303 | 24.757 | −9.359 | 70.711 | 1.00 | 38.19 | B |
| ATOM | 2154 | O | VAL | 303 | 24.495 | −10.506 | 70.368 | 1.00 | 39.57 | B |
| ATOM | 2155 | N | GLU | 304 | 24.597 | −8.928 | 71.957 | 1.00 | 39.43 | B |
| ATOM | 2156 | CA | GLU | 304 | 24.129 | −9.796 | 73.032 | 1.00 | 40.38 | B |
| ATOM | 2157 | CB | GLU | 304 | 24.768 | −9.359 | 74.350 | 1.00 | 41.03 | B |
| ATOM | 2158 | CG | GLU | 304 | 26.290 | −9.464 | 74.347 | 1.00 | 42.14 | B |
| ATOM | 2159 | CD | GLU | 304 | 26.889 | −9.210 | 75.713 | 1.00 | 43.89 | B |
| ATOM | 2160 | OE1 | GLU | 304 | 28.116 | −9.390 | 75.879 | 1.00 | 42.77 | B |
| ATOM | 2161 | OE2 | GLU | 304 | 26.127 | −8.827 | 76.625 | 1.00 | 45.66 | B |
| ATOM | 2162 | C | GLU | 304 | 22.612 | −9.817 | 73.179 | 1.00 | 41.20 | B |
| ATOM | 2163 | O | GLU | 304 | 22.071 | −10.477 | 74.062 | 1.00 | 39.68 | B |
| ATOM | 2164 | N | ARG | 305 | 21.932 | −9.088 | 72.305 | 1.00 | 44.11 | B |
| ATOM | 2165 | CA | ARG | 305 | 20.474 | −9.004 | 72.310 | 1.00 | 46.91 | B |
| ATOM | 2166 | CB | ARG | 305 | 19.835 | −10.408 | 71.997 | 1.00 | 48.72 | B |
| ATOM | 2167 | CG | ARG | 305 | 20.520 | −11.222 | 70.897 | 1.00 | 52.86 | B |
| ATOM | 2168 | CD | ARG | 305 | 20.686 | −10.461 | 69.579 | 1.00 | 56.32 | B |
| ATOM | 2169 | NE | ARG | 305 | 21.395 | −11.268 | 68.582 | 1.00 | 59.70 | B |
| ATOM | 2170 | CZ | ARG | 305 | 21.970 | −10.782 | 67.483 | 1.00 | 61.81 | B |
| ATOM | 2171 | NH1 | ARG | 305 | 21.926 | −9.479 | 67.221 | 1.00 | 61.95 | B |
| ATOM | 2172 | NH2 | ARG | 305 | 22.605 | −11.601 | 66.649 | 1.00 | 61.81 | B |
| ATOM | 2173 | C | ARG | 305 | 19.890 | −8.469 | 73.620 | 1.00 | 47.13 | B |
| ATOM | 2174 | O | ARG | 305 | 18.784 | −8.840 | 73.996 | 1.00 | 48.14 | B |
| ATOM | 2175 | N | THR | 306 | 20.621 | −7.599 | 74.311 | 1.00 | 48.36 | B |
| ATOM | 2176 | CA | THR | 306 | 20.135 | −7.027 | 75.568 | 1.00 | 49.45 | B |
| ATOM | 2177 | CB | THR | 306 | 21.275 | −6.367 | 76.356 | 1.00 | 49.08 | B |
| ATOM | 2178 | OG1 | THR | 306 | 22.429 | −7.214 | 76.326 | 1.00 | 49.36 | B |
| ATOM | 2179 | CG2 | THR | 306 | 20.862 | −6.155 | 77.802 | 1.00 | 48.92 | B |
| ATOM | 2180 | C | THR | 306 | 19.066 | −5.972 | 75.262 | 1.00 | 50.64 | B |
| ATOM | 2181 | O | THR | 306 | 19.275 | −5.091 | 74.428 | 1.00 | 51.81 | B |
| ATOM | 2182 | N | PRO | 307 | 17.910 | −6.044 | 75.942 | 1.00 | 51.76 | B |
| ATOM | 2183 | CD | PRO | 307 | 17.651 | −6.959 | 77.068 | 1.00 | 52.91 | B |
| ATOM | 2184 | CA | PRO | 307 | 16.779 | −5.119 | 75.761 | 1.00 | 52.01 | B |
| ATOM | 2185 | CB | PRO | 307 | 15.945 | −5.358 | 76.995 | 1.00 | 52.53 | B |
| ATOM | 2186 | CG | PRO | 307 | 16.158 | −6.818 | 77.257 | 1.00 | 53.28 | B |
| ATOM | 2187 | C | PRO | 307 | 17.124 | −3.638 | 75.585 | 1.00 | 51.42 | B |
| ATOM | 2188 | O | PRO | 307 | 16.624 | −2.983 | 74.664 | 1.00 | 51.33 | B |
| ATOM | 2189 | N | HIS | 308 | 17.973 | −3.115 | 76.466 | 1.00 | 49.88 | B |
| ATOM | 2190 | CA | HIS | 308 | 18.359 | −1.711 | 76.410 | 1.00 | 47.29 | B |
| ATOM | 2191 | CB | HIS | 308 | 18.432 | −1.141 | 77.832 | 1.00 | 50.27 | B |
| ATOM | 2192 | CG | HIS | 308 | 18.812 | 0.306 | 77.877 | 1.00 | 54.50 | B |
| ATOM | 2193 | CD2 | HIS | 308 | 19.992 | 0.909 | 78.158 | 1.00 | 55.48 | B |
| ATOM | 2194 | ND1 | HIS | 308 | 17.931 | 1.318 | 77.559 | 1.00 | 55.94 | B |
| ATOM | 2195 | CE1 | HIS | 308 | 18.552 | 2.482 | 77.641 | 1.00 | 56.20 | B |
| ATOM | 2196 | NE2 | HIS | 308 | 19.804 | 2.262 | 78.003 | 1.00 | 56.35 | B |
| ATOM | 2197 | C | HIS | 308 | 19.685 | −1.445 | 75.690 | 1.00 | 43.71 | B |
| ATOM | 2198 | O | HIS | 308 | 20.709 | −2.061 | 75.991 | 1.00 | 43.17 | B |
| ATOM | 2199 | N | VAL | 309 | 19.649 | −0.517 | 74.737 | 1.00 | 39.63 | B |
| ATOM | 2200 | CA | VAL | 309 | 20.829 | −0.117 | 73.964 | 1.00 | 34.96 | B |
| ATOM | 2201 | CB | VAL | 309 | 20.561 | −0.206 | 72.449 | 1.00 | 34.96 | B |
| ATOM | 2202 | CG1 | VAL | 309 | 21.858 | 0.013 | 71.675 | 1.00 | 34.27 | B |
| ATOM | 2203 | CG2 | VAL | 309 | 19.934 | −1.548 | 72.114 | 1.00 | 32.68 | B |
| ATOM | 2204 | C | VAL | 309 | 21.086 | 1.344 | 74.336 | 1.00 | 31.77 | B |
| ATOM | 2205 | O | VAL | 309 | 20.237 | 2.204 | 74.102 | 1.00 | 30.77 | B |
| ATOM | 2206 | N | PRO | 310 | 22.266 | 1.642 | 74.906 | 1.00 | 29.55 | B |
| ATOM | 2207 | CD | PRO | 310 | 23.347 | 0.670 | 75.171 | 1.00 | 27.65 | B |
| ATOM | 2208 | CA | PRO | 310 | 22.652 | 2.997 | 75.335 | 1.00 | 29.03 | B |
| ATOM | 2209 | CB | PRO | 310 | 23.856 | 2.732 | 76.230 | 1.00 | 29.20 | B |
| ATOM | 2210 | CG | PRO | 310 | 24.518 | 1.555 | 75.539 | 1.00 | 27.40 | B |
| ATOM | 2211 | C | PRO | 310 | 22.949 | 4.064 | 74.268 | 1.00 | 28.13 | B |
| ATOM | 2212 | O | PRO | 310 | 23.960 | 4.760 | 74.357 | 1.00 | 27.93 | B |
| ATOM | 2213 | N | TYR | 311 | 22.064 | 4.198 | 73.284 | 1.00 | 27.73 | B |
| ATOM | 2214 | CA | TYR | 311 | 22.217 | 5.175 | 72.203 | 1.00 | 28.46 | B |
| ATOM | 2215 | CB | TYR | 311 | 20.949 | 5.195 | 71.291 | 1.00 | 29.00 | B |
| ATOM | 2216 | CG | TYR | 311 | 20.724 | 3.960 | 70.450 | 1.00 | 32.30 | B |
| ATOM | 2217 | CD1 | TYR | 311 | 21.600 | 3.631 | 69.413 | 1.00 | 32.05 | B |
| ATOM | 2218 | CE1 | TYR | 311 | 21.393 | 2.492 | 68.628 | 1.00 | 34.37 | B |
| ATOM | 2219 | CD2 | TYR | 311 | 19.627 | 3.119 | 70.686 | 1.00 | 31.31 | B |
| ATOM | 2220 | CE2 | TYR | 311 | 19.411 | 1.979 | 69.908 | 1.00 | 32.07 | B |
| ATOM | 2221 | CZ | TYR | 311 | 20.299 | 1.669 | 68.882 | 1.00 | 34.42 | B |
| ATOM | 2222 | OH | TYR | 311 | 20.120 | 0.531 | 68.122 | 1.00 | 35.43 | B |
| ATOM | 2223 | C | TYR | 311 | 22.458 | 6.611 | 72.678 | 1.00 | 28.67 | B |
| ATOM | 2224 | O | TYR | 311 | 23.343 | 7.296 | 72.177 | 1.00 | 27.07 | B |
| ATOM | 2225 | N | ARG | 312 | 21.652 | 7.059 | 73.635 | 1.00 | 29.15 | B |
| ATOM | 2226 | CA | ARG | 312 | 21.716 | 8.425 | 74.143 | 1.00 | 29.95 | B |
| ATOM | 2227 | CB | ARG | 312 | 20.481 | 8.724 | 74.961 | 1.00 | 32.31 | B |
| ATOM | 2228 | CG | ARG | 312 | 19.189 | 8.626 | 74.196 | 1.00 | 36.65 | B |
| ATOM | 2229 | CD | ARG | 312 | 18.046 | 8.529 | 75.169 | 1.00 | 40.81 | B |
| ATOM | 2230 | NE | ARG | 312 | 16.862 | 7.919 | 74.577 | 1.00 | 43.18 | B |
| ATOM | 2231 | CZ | ARG | 312 | 15.951 | 7.251 | 75.278 | 1.00 | 45.73 | B |
| ATOM | 2232 | NH1 | ARG | 312 | 16.100 | 7.108 | 76.597 | 1.00 | 44.15 | B |
| ATOM | 2233 | NH2 | ARG | 312 | 14.888 | 6.737 | 74.664 | 1.00 | 45.91 | B |
| ATOM | 2234 | C | ARG | 312 | 22.926 | 8.811 | 74.969 | 1.00 | 28.83 | B |
| ATOM | 2235 | O | ARG | 312 | 23.104 | 9.991 | 75.276 | 1.00 | 29.69 | B |
| ATOM | 2236 | N | GLU | 313 | 23.755 | 7.843 | 75.340 | 1.00 | 26.62 | B |
| ATOM | 2237 | CA | GLU | 313 | 24.917 | 8.160 | 76.153 | 1.00 | 22.31 | B |
| ATOM | 2238 | CB | GLU | 313 | 25.419 | 6.929 | 76.814 | 1.00 | 22.37 | B |
| ATOM | 2239 | CG | GLU | 313 | 24.550 | 6.521 | 77.994 | 1.00 | 24.92 | B |
| ATOM | 2240 | CD | GLU | 313 | 24.871 | 5.136 | 78.554 | 1.00 | 26.13 | B |
| ATOM | 2241 | OE1 | GLU | 313 | 26.060 | 4.823 | 78.755 | 1.00 | 27.91 | B |
| ATOM | 2242 | OE2 | GLU | 313 | 23.926 | 4.365 | 78.813 | 1.00 | 27.77 | B |
| ATOM | 2243 | C | GLU | 313 | 26.031 | 8.873 | 75.403 | 1.00 | 21.16 | B |
| ATOM | 2244 | O | GLU | 313 | 27.096 | 9.122 | 75.963 | 1.00 | 21.76 | B |
| ATOM | 2245 | N | SER | 314 | 25.789 | 9.222 | 74.144 | 1.00 | 18.52 | B |
| ATOM | 2246 | CA | SER | 314 | 26.796 | 9.935 | 73.375 | 1.00 | 19.81 | B |
| ATOM | 2247 | CB | SER | 314 | 27.966 | 8.992 | 72.968 | 1.00 | 20.10 | B |
| ATOM | 2248 | OG | SER | 314 | 27.731 | 8.382 | 71.710 | 1.00 | 19.29 | B |
| ATOM | 2249 | C | SER | 314 | 26.206 | 10.583 | 72.130 | 1.00 | 20.60 | B |
| ATOM | 2250 | O | SER | 314 | 25.198 | 10.126 | 71.597 | 1.00 | 19.90 | B |
| ATOM | 2251 | N | LYS | 315 | 26.854 | 11.654 | 71.676 | 1.00 | 20.92 | B |
| ATOM | 2252 | CA | LYS | 315 | 26.412 | 12.395 | 70.504 | 1.00 | 20.48 | B |
| ATOM | 2253 | CB | LYS | 315 | 27.264 | 13.689 | 70.329 | 1.00 | 20.26 | B |
| ATOM | 2254 | CG | LYS | 315 | 27.318 | 14.572 | 71.556 | 1.00 | 19.73 | B |
| ATOM | 2255 | CD | LYS | 315 | 25.936 | 14.893 | 72.074 | 1.00 | 22.19 | B |
| ATOM | 2256 | CE | LYS | 315 | 25.984 | 15.989 | 73.129 | 1.00 | 23.41 | B |
| ATOM | 2257 | NZ | LYS | 315 | 26.408 | 17.293 | 72.528 | 1.00 | 26.09 | B |
| ATOM | 2258 | C | LYS | 315 | 26.513 | 11.560 | 69.239 | 1.00 | 19.78 | B |
| ATOM | 2259 | O | LYS | 315 | 25.626 | 11.614 | 68.373 | 1.00 | 20.29 | B |
| ATOM | 2260 | N | LEU | 316 | 27.598 | 10.796 | 69.130 | 1.00 | 17.65 | B |
| ATOM | 2261 | CA | LEU | 316 | 27.808 | 9.962 | 67.955 | 1.00 | 17.80 | B |
| ATOM | 2262 | CB | LEU | 316 | 29.209 | 9.245 | 68.013 | 1.00 | 16.46 | B |
| ATOM | 2263 | CG | LEU | 316 | 29.602 | 8.339 | 66.775 | 1.00 | 15.01 | B |
| ATOM | 2264 | CD1 | LEU | 316 | 29.683 | 9.151 | 65.507 | 1.00 | 14.12 | B |
| ATOM | 2265 | CD2 | LEU | 316 | 30.937 | 7.695 | 67.030 | 1.00 | 17.53 | B |
| ATOM | 2266 | C | LEU | 316 | 26.698 | 8.926 | 67.798 | 1.00 | 17.14 | B |
| ATOM | 2267 | O | LEU | 316 | 26.060 | 8.854 | 66.742 | 1.00 | 17.17 | B |
| ATOM | 2268 | N | THR | 317 | 26.462 | 8.137 | 68.844 | 1.00 | 17.69 | B |
| ATOM | 2269 | CA | THR | 317 | 25.439 | 7.106 | 68.777 | 1.00 | 19.04 | B |
| ATOM | 2270 | CB | THR | 317 | 25.525 | 6.124 | 69.966 | 1.00 | 21.44 | B |
| ATOM | 2271 | OG1 | THR | 317 | 25.617 | 6.848 | 71.198 | 1.00 | 21.96 | B |
| ATOM | 2272 | CG2 | THR | 317 | 26.743 | 5.206 | 69.804 | 1.00 | 21.41 | B |
| ATOM | 2273 | C | THR | 317 | 24.031 | 7.659 | 68.659 | 1.00 | 18.09 | B |
| ATOM | 2274 | O | THR | 317 | 23.155 | 6.990 | 68.130 | 1.00 | 17.17 | B |
| ATOM | 2275 | N | ARG | 318 | 23.800 | 8.877 | 69.134 | 1.00 | 19.16 | B |
| ATOM | 2276 | CA | ARG | 318 | 22.469 | 9.460 | 68.986 | 1.00 | 20.49 | B |
| ATOM | 2277 | CB | ARG | 318 | 22.283 | 10.654 | 69.927 | 1.00 | 22.85 | B |
| ATOM | 2278 | CG | ARG | 318 | 22.155 | 10.218 | 71.387 | 1.00 | 28.27 | B |
| ATOM | 2279 | CD | ARG | 318 | 21.942 | 11.375 | 72.318 | 1.00 | 31.62 | B |
| ATOM | 2280 | NE | ARG | 318 | 20.929 | 12.277 | 71.788 | 1.00 | 39.60 | B |
| ATOM | 2281 | CZ | ARG | 318 | 20.361 | 13.261 | 72.479 | 1.00 | 40.99 | B |
| ATOM | 2282 | NH1 | ARG | 318 | 20.703 | 13.474 | 73.746 | 1.00 | 41.19 | B |
| ATOM | 2283 | NH2 | ARG | 318 | 19.454 | 14.034 | 71.894 | 1.00 | 41.05 | B |
| ATOM | 2284 | C | ARG | 318 | 22.288 | 9.873 | 67.525 | 1.00 | 20.16 | B |
| ATOM | 2285 | O | ARG | 318 | 21.237 | 9.648 | 66.929 | 1.00 | 21.26 | B |
| ATOM | 2286 | N | ILE | 319 | 23.332 | 10.435 | 66.932 | 1.00 | 18.27 | B |
| ATOM | 2287 | CA | ILE | 319 | 23.255 | 10.843 | 65.539 | 1.00 | 18.18 | B |
| ATOM | 2288 | CB | ILE | 319 | 24.505 | 11.665 | 65.132 | 1.00 | 17.80 | B |
| ATOM | 2289 | CG2 | ILE | 319 | 24.482 | 11.913 | 63.619 | 1.00 | 17.11 | B |
| ATOM | 2290 | CG1 | ILE | 319 | 24.561 | 13.006 | 65.928 | 1.00 | 17.07 | B |
| ATOM | 2291 | CD1 | ILE | 319 | 25.901 | 13.727 | 65.838 | 1.00 | 14.30 | B |
| ATOM | 2292 | C | ILE | 319 | 23.134 | 9.663 | 64.550 | 1.00 | 18.77 | B |
| ATOM | 2293 | O | ILE | 319 | 22.397 | 9.753 | 63.569 | 1.00 | 16.28 | B |
| ATOM | 2294 | N | LEU | 320 | 23.860 | 8.571 | 64.808 | 1.00 | 18.72 | B |
| ATOM | 2295 | CA | LEU | 320 | 23.874 | 7.415 | 63.905 | 1.00 | 18.52 | B |
| ATOM | 2296 | CB | LEU | 320 | 25.323 | 7.003 | 63.621 | 1.00 | 14.27 | B |
| ATOM | 2297 | CG | LEU | 320 | 26.321 | 8.000 | 63.025 | 1.00 | 16.38 | B |
| ATOM | 2298 | CD1 | LEU | 320 | 27.707 | 7.354 | 63.017 | 1.00 | 13.61 | B |
| ATOM | 2299 | CD2 | LEU | 320 | 25.905 | 8.426 | 61.605 | 1.00 | 14.32 | B |
| ATOM | 2300 | C | LEU | 320 | 23.113 | 6.159 | 64.354 | 1.00 | 21.16 | B |
| ATOM | 2301 | O | LEU | 320 | 23.308 | 5.087 | 63.780 | 1.00 | 21.77 | B |
| ATOM | 2302 | N | GLN | 321 | 22.249 | 6.277 | 65.357 | 1.00 | 22.79 | B |
| ATOM | 2303 | CA | GLN | 321 | 21.519 | 5.114 | 65.848 | 1.00 | 25.68 | B |
| ATOM | 2304 | CB | GLN | 321 | 20.531 | 5.524 | 66.954 | 1.00 | 28.52 | B |
| ATOM | 2305 | CG | GLN | 321 | 19.448 | 6.490 | 66.535 | 1.00 | 32.15 | B |
| ATOM | 2306 | CD | GLN | 321 | 18.539 | 6.843 | 67.700 | 1.00 | 35.99 | B |
| ATOM | 2307 | OE1 | GLN | 321 | 17.953 | 5.954 | 68.332 | 1.00 | 33.89 | B |
| ATOM | 2308 | NE2 | GLN | 321 | 18.417 | 8.144 | 67.997 | 1.00 | 36.73 | B |
| ATOM | 2309 | C | GLN | 321 | 20.790 | 4.254 | 64.813 | 1.00 | 25.53 | B |
| ATOM | 2310 | O | GLN | 321 | 20.625 | 3.056 | 65.029 | 1.00 | 25.73 | B |
| ATOM | 2311 | N | ASP | 322 | 20.353 | 4.837 | 63.701 | 1.00 | 26.46 | B |
| ATOM | 2312 | CA | ASP | 322 | 19.659 | 4.040 | 62.695 | 1.00 | 28.33 | B |
| ATOM | 2313 | CB | ASP | 322 | 18.913 | 4.934 | 61.681 | 1.00 | 29.02 | B |
| ATOM | 2314 | CG | ASP | 322 | 17.894 | 4.152 | 60.847 | 1.00 | 30.51 | B |
| ATOM | 2315 | OD1 | ASP | 322 | 17.880 | 4.308 | 59.604 | 1.00 | 31.51 | B |
| ATOM | 2316 | OD2 | ASP | 322 | 17.100 | 3.384 | 61.434 | 1.00 | 29.46 | B |
| ATOM | 2317 | C | ASP | 322 | 20.661 | 3.152 | 61.959 | 1.00 | 29.44 | B |
| ATOM | 2318 | O | ASP | 322 | 20.284 | 2.195 | 61.280 | 1.00 | 29.55 | B |
| ATOM | 2319 | N | SER | 323 | 21.943 | 3.480 | 62.095 | 1.00 | 29.59 | B |
| ATOM | 2320 | CA | SER | 323 | 22.999 | 2.705 | 61.458 | 1.00 | 28.78 | B |
| ATOM | 2321 | CB | SER | 323 | 24.172 | 3.594 | 61.165 | 1.00 | 27.31 | B |
| ATOM | 2322 | OG | SER | 323 | 23.845 | 4.545 | 60.178 | 1.00 | 26.34 | B |
| ATOM | 2323 | C | SER | 323 | 23.453 | 1.519 | 62.322 | 1.00 | 29.30 | B |
| ATOM | 2324 | O | SER | 323 | 24.234 | 0.687 | 61.875 | 1.00 | 28.51 | B |
| ATOM | 2325 | N | LEU | 324 | 22.967 | 1.445 | 63.558 | 1.00 | 30.19 | B |
| ATOM | 2326 | CA | LEU | 324 | 23.338 | 0.354 | 64.451 | 1.00 | 30.51 | B |
| ATOM | 2327 | CB | LEU | 324 | 24.110 | 0.893 | 65.662 | 1.00 | 30.62 | B |
| ATOM | 2328 | CG | LEU | 324 | 25.577 | 1.365 | 65.474 | 1.00 | 29.76 | B |
| ATOM | 2329 | CD1 | LEU | 324 | 25.670 | 2.412 | 64.401 | 1.00 | 31.76 | B |
| ATOM | 2330 | CD2 | LEU | 324 | 26.085 | 1.928 | 66.775 | 1.00 | 28.62 | B |
| ATOM | 2331 | C | LEU | 324 | 22.113 | −0.419 | 64.927 | 1.00 | 31.44 | B |
| ATOM | 2332 | O | LEU | 324 | 21.611 | −0.184 | 66.026 | 1.00 | 32.71 | B |
| ATOM | 2333 | N | GLY | 325 | 21.642 | −1.347 | 64.095 | 1.00 | 31.87 | B |
| ATOM | 2334 | CA | GLY | 325 | 20.479 | −2.148 | 64.444 | 1.00 | 30.03 | B |
| ATOM | 2335 | C | GLY | 325 | 19.190 | −1.440 | 64.082 | 1.00 | 29.89 | B |
| ATOM | 2336 | O | GLY | 325 | 18.160 | −1.636 | 64.727 | 1.00 | 29.38 | B |
| ATOM | 2337 | N | GLY | 326 | 19.253 | −0.614 | 63.042 | 1.00 | 29.59 | B |
| ATOM | 2338 | CA | GLY | 326 | 18.092 | 0.139 | 62.603 | 1.00 | 27.99 | B |
| ATOM | 2339 | C | GLY | 326 | 17.706 | −0.236 | 61.193 | 1.00 | 27.84 | B |
| ATOM | 2340 | O | GLY | 326 | 17.896 | −1.378 | 60.811 | 1.00 | 28.56 | B |
| ATOM | 2341 | N | ARG | 327 | 17.197 | 0.719 | 60.418 | 1.00 | 26.60 | B |
| ATOM | 2342 | CA | ARG | 327 | 16.763 | 0.456 | 59.046 | 1.00 | 27.36 | B |
| ATOM | 2343 | CB | ARG | 327 | 15.451 | 1.234 | 58.745 | 1.00 | 30.55 | B |
| ATOM | 2344 | CG | ARG | 327 | 14.534 | 1.451 | 59.943 | 1.00 | 34.58 | B |
| ATOM | 2345 | CD | ARG | 327 | 13.775 | 0.198 | 60.367 | 1.00 | 40.44 | B |
| ATOM | 2346 | NE | ARG | 327 | 12.359 | 0.271 | 60.014 | 1.00 | 43.41 | B |
| ATOM | 2347 | CZ | ARG | 327 | 11.898 | 0.209 | 58.768 | 1.00 | 47.99 | B |
| ATOM | 2348 | NH1 | ARG | 327 | 12.741 | 0.071 | 57.751 | 1.00 | 49.86 | B |
| ATOM | 2349 | NH2 | ARG | 327 | 10.592 | 0.285 | 58.535 | 1.00 | 48.98 | B |
| ATOM | 2350 | C | ARG | 327 | 17.796 | 0.811 | 57.967 | 1.00 | 27.20 | B |
| ATOM | 2351 | O | ARG | 327 | 17.521 | 0.680 | 56.775 | 1.00 | 27.07 | B |
| ATOM | 2352 | N | THR | 328 | 18.977 | 1.257 | 58.379 | 1.00 | 26.89 | B |
| ATOM | 2353 | CA | THR | 328 | 20.028 | 1.646 | 57.441 | 1.00 | 25.49 | B |
| ATOM | 2354 | CB | THR | 328 | 20.870 | 2.813 | 58.024 | 1.00 | 27.20 | B |
| ATOM | 2355 | OG1 | THR | 328 | 20.024 | 3.944 | 58.252 | 1.00 | 29.46 | B |
| ATOM | 2356 | CG2 | THR | 328 | 21.992 | 3.210 | 57.072 | 1.00 | 26.15 | B |
| ATOM | 2357 | C | THR | 328 | 20.974 | 0.492 | 57.125 | 1.00 | 24.96 | B |
| ATOM | 2358 | O | THR | 328 | 21.238 | −0.346 | 57.984 | 1.00 | 24.98 | B |
| ATOM | 2359 | N | ARG | 329 | 21.465 | 0.431 | 55.890 | 1.00 | 23.74 | B |
| ATOM | 2360 | CA | ARG | 329 | 22.426 | −0.610 | 55.543 | 1.00 | 24.57 | B |
| ATOM | 2361 | CB | ARG | 329 | 22.551 | −0.842 | 54.014 | 1.00 | 26.29 | B |
| ATOM | 2362 | CG | ARG | 329 | 23.421 | −2.071 | 53.721 | 1.00 | 31.07 | B |
| ATOM | 2363 | CD | ARG | 329 | 24.277 | −1.980 | 52.461 | 1.00 | 34.15 | B |
| ATOM | 2364 | NE | ARG | 329 | 23.590 | −2.447 | 51.259 | 1.00 | 37.59 | B |
| ATOM | 2365 | CZ | ARG | 329 | 24.217 | −2.885 | 50.168 | 1.00 | 38.17 | B |
| ATOM | 2366 | NH1 | ARG | 329 | 25.547 | −2.923 | 50.124 | 1.00 | 38.35 | B |
| ATOM | 2367 | NH2 | ARG | 329 | 23.513 | −3.284 | 49.119 | 1.00 | 36.37 | B |
| ATOM | 2368 | C | ARG | 329 | 23.761 | −0.102 | 56.061 | 1.00 | 22.51 | B |
| ATOM | 2369 | O | ARG | 329 | 24.174 | 1.012 | 55.741 | 1.00 | 21.91 | B |
| ATOM | 2370 | N | THR | 330 | 24.431 | −0.919 | 56.856 | 1.00 | 21.40 | B |
| ATOM | 2371 | CA | THR | 330 | 25.704 | −0.529 | 57.433 | 1.00 | 21.18 | B |
| ATOM | 2372 | CB | THR | 330 | 25.610 | −0.435 | 58.971 | 1.00 | 20.58 | B |
| ATOM | 2373 | OG1 | THR | 330 | 24.666 | 0.581 | 59.317 | 1.00 | 22.60 | B |
| ATOM | 2374 | CG2 | THR | 330 | 26.962 | −0.099 | 59.581 | 1.00 | 17.89 | B |
| ATOM | 2375 | C | THR | 330 | 26.837 | −1.471 | 57.085 | 1.00 | 21.32 | B |
| ATOM | 2376 | O | THR | 330 | 26.673 | −2.691 | 57.001 | 1.00 | 19.41 | B |
| ATOM | 2377 | N | SER | 331 | 28.002 | −0.872 | 56.902 | 1.00 | 21.49 | B |
| ATOM | 2378 | CA | SER | 331 | 29.200 | −1.602 | 56.574 | 1.00 | 21.39 | B |
| ATOM | 2379 | CB | SER | 331 | 29.469 | −1.473 | 55.084 | 1.00 | 22.34 | B |
| ATOM | 2380 | OG | SER | 331 | 30.537 | −2.313 | 54.694 | 1.00 | 26.49 | B |
| ATOM | 2381 | C | SER | 331 | 30.340 | −1.001 | 57.391 | 1.00 | 20.49 | B |
| ATOM | 2382 | O | SER | 331 | 30.418 | 0.208 | 57.565 | 1.00 | 21.48 | B |
| ATOM | 2383 | N | ILE | 332 | 31.213 | −1.849 | 57.911 | 1.00 | 18.89 | B |
| ATOM | 2384 | CA | ILE | 332 | 32.341 | −1.371 | 58.695 | 1.00 | 15.95 | B |
| ATOM | 2385 | CB | ILE | 332 | 32.321 | −1.936 | 60.135 | 1.00 | 15.17 | B |
| ATOM | 2386 | CG2 | ILE | 332 | 33.621 | −1.568 | 60.854 | 1.00 | 12.52 | B |
| ATOM | 2387 | CG1 | ILE | 332 | 31.091 | −1.447 | 60.882 | 1.00 | 11.58 | B |
| ATOM | 2388 | CD1 | ILE | 332 | 30.932 | −2.097 | 62.247 | 1.00 | 7.00 | B |
| ATOM | 2389 | C | ILE | 332 | 33.650 | −1.818 | 58.063 | 1.00 | 15.41 | B |
| ATOM | 2390 | O | ILE | 332 | 33.802 | −2.980 | 57.687 | 1.00 | 12.48 | B |
| ATOM | 2391 | N | ILE | 333 | 34.591 | −0.888 | 57.948 | 1.00 | 16.21 | B |
| ATOM | 2392 | CA | ILE | 333 | 35.899 | −1.203 | 57.411 | 1.00 | 16.71 | B |
| ATOM | 2393 | CB | ILE | 333 | 36.310 | −0.266 | 56.273 | 1.00 | 16.82 | B |
| ATOM | 2394 | CG2 | ILE | 333 | 37.616 | −0.744 | 55.675 | 1.00 | 15.94 | B |
| ATOM | 2395 | CG1 | ILE | 333 | 35.242 | −0.259 | 55.169 | 1.00 | 16.68 | B |
| ATOM | 2396 | CD1 | ILE | 333 | 35.557 | 0.705 | 54.012 | 1.00 | 15.18 | B |
| ATOM | 2397 | C | ILE | 333 | 36.860 | −1.021 | 58.561 | 1.00 | 18.56 | B |
| ATOM | 2398 | O | ILE | 333 | 37.074 | 0.104 | 59.032 | 1.00 | 21.41 | B |
| ATOM | 2399 | N | ALA | 334 | 37.411 | −2.137 | 59.035 | 1.00 | 20.14 | B |
| ATOM | 2400 | CA | ALA | 334 | 38.360 | −2.125 | 60.147 | 1.00 | 19.94 | B |
| ATOM | 2401 | CB | ALA | 334 | 38.182 | −3.362 | 61.020 | 1.00 | 18.30 | B |
| ATOM | 2402 | C | ALA | 334 | 39.756 | −2.096 | 59.550 | 1.00 | 20.34 | B |
| ATOM | 2403 | O | ALA | 334 | 40.135 | −2.989 | 58.790 | 1.00 | 20.44 | B |
| ATOM | 2404 | N | THR | 335 | 40.514 | −1.062 | 59.897 | 1.00 | 19.08 | B |
| ATOM | 2405 | CA | THR | 335 | 41.853 | −0.901 | 59.369 | 1.00 | 19.70 | B |
| ATOM | 2406 | CB | THR | 335 | 42.106 | 0.584 | 59.008 | 1.00 | 21.15 | B |
| ATOM | 2407 | OG1 | THR | 335 | 41.876 | 1.409 | 60.157 | 1.00 | 24.31 | B |
| ATOM | 2408 | CG2 | THR | 335 | 41.158 | 1.026 | 57.905 | 1.00 | 21.90 | B |
| ATOM | 2409 | C | THR | 335 | 42.907 | −1.403 | 60.351 | 1.00 | 19.67 | B |
| ATOM | 2410 | O | THR | 335 | 42.796 | −1.190 | 61.559 | 1.00 | 20.81 | B |
| ATOM | 2411 | N | ILE | 336 | 43.924 | −2.085 | 59.833 | 1.00 | 19.06 | B |
| ATOM | 2412 | CA | ILE | 336 | 44.991 | −2.618 | 60.680 | 1.00 | 19.16 | B |
| ATOM | 2413 | CB | ILE | 336 | 44.845 | −4.147 | 60.882 | 1.00 | 18.20 | B |
| ATOM | 2414 | CG2 | ILE | 336 | 43.519 | −4.470 | 61.562 | 1.00 | 17.20 | B |
| ATOM | 2415 | CG1 | ILE | 336 | 44.933 | −4.857 | 59.564 | 1.00 | 15.56 | B |
| ATOM | 2416 | CD1 | ILE | 336 | 44.926 | −6.371 | 59.697 | 1.00 | 16.09 | B |
| ATOM | 2417 | C | ILE | 336 | 46.388 | −2.343 | 60.116 | 1.00 | 19.85 | B |
| ATOM | 2418 | O | ILE | 336 | 46.547 | −1.995 | 58.945 | 1.00 | 20.63 | B |
| ATOM | 2419 | N | SER | 337 | 47.395 | −2.487 | 60.970 | 1.00 | 21.82 | B |
| ATOM | 2420 | CA | SER | 337 | 48.788 | −2.277 | 60.576 | 1.00 | 23.86 | B |
| ATOM | 2421 | CB | SER | 337 | 49.514 | −1.430 | 61.611 | 1.00 | 22.35 | B |
| ATOM | 2422 | OG | SER | 337 | 50.551 | −2.165 | 62.229 | 1.00 | 19.41 | B |
| ATOM | 2423 | C | SER | 337 | 49.507 | −3.622 | 60.458 | 1.00 | 26.10 | B |
| ATOM | 2424 | O | SER | 337 | 49.133 | −4.597 | 61.119 | 1.00 | 25.43 | B |
| ATOM | 2425 | N | PRO | 338 | 50.543 | −3.692 | 59.606 | 1.00 | 26.45 | B |
| ATOM | 2426 | CD | PRO | 338 | 50.873 | −2.755 | 58.518 | 1.00 | 26.43 | B |
| ATOM | 2427 | CA | PRO | 338 | 51.287 | −4.943 | 59.441 | 1.00 | 27.75 | B |
| ATOM | 2428 | CB | PRO | 338 | 51.703 | −4.893 | 58.009 | 1.00 | 25.91 | B |
| ATOM | 2429 | CG | PRO | 338 | 52.043 | −3.453 | 57.835 | 1.00 | 26.04 | B |
| ATOM | 2430 | C | PRO | 338 | 52.493 | −5.016 | 60.366 | 1.00 | 28.99 | B |
| ATOM | 2431 | O | PRO | 338 | 53.304 | −5.929 | 60.250 | 1.00 | 30.27 | B |
| ATOM | 2432 | N | ALA | 339 | 52.615 | −4.057 | 61.280 | 1.00 | 29.91 | B |
| ATOM | 2433 | CA | ALA | 339 | 53.765 | −4.024 | 62.184 | 1.00 | 31.92 | B |
| ATOM | 2434 | CB | ALA | 339 | 54.076 | −2.582 | 62.598 | 1.00 | 32.09 | B |
| ATOM | 2435 | C | ALA | 339 | 53.576 | −4.884 | 63.415 | 1.00 | 31.91 | B |
| ATOM | 2436 | O | ALA | 339 | 52.483 | −4.965 | 63.959 | 1.00 | 34.29 | B |
| ATOM | 2437 | N | SER | 340 | 54.651 | −5.525 | 63.856 | 1.00 | 31.24 | B |
| ATOM | 2438 | CA | SER | 340 | 54.580 | −6.374 | 65.030 | 1.00 | 29.08 | B |
| ATOM | 2439 | CB | SER | 340 | 55.877 | −7.280 | 65.138 | 1.00 | 29.57 | B |
| ATOM | 2440 | OG | SER | 340 | 57.053 | −6.513 | 65.327 | 1.00 | 28.10 | B |
| ATOM | 2441 | C | SER | 340 | 54.396 | −5.555 | 66.307 | 1.00 | 28.00 | B |
| ATOM | 2442 | O | SER | 340 | 53.844 | −6.046 | 67.280 | 1.00 | 28.20 | B |
| ATOM | 2443 | N | LEU | 341 | 54.852 | −4.308 | 66.309 | 1.00 | 28.24 | B |
| ATOM | 2444 | CA | LEU | 341 | 54.715 | −3.471 | 67.493 | 1.00 | 28.05 | B |
| ATOM | 2445 | CB | LEU | 341 | 55.742 | −2.306 | 67.463 | 1.00 | 29.43 | B |
| ATOM | 2446 | CG | LEU | 341 | 55.315 | −0.861 | 67.190 | 1.00 | 30.31 | B |
| ATOM | 2447 | CD1 | LEU | 341 | 56.404 | 0.084 | 67.690 | 1.00 | 28.26 | B |
| ATOM | 2448 | CD2 | LEU | 341 | 55.065 | −0.659 | 65.707 | 1.00 | 31.94 | B |
| ATOM | 2449 | C | LEU | 341 | 53.290 | −2.936 | 67.647 | 1.00 | 28.81 | B |
| ATOM | 2450 | O | LEU | 341 | 52.954 | −2.305 | 68.650 | 1.00 | 28.00 | B |
| ATOM | 2451 | N | ASN | 342 | 52.450 | −3.209 | 66.656 | 1.00 | 28.88 | B |
| ATOM | 2452 | CA | ASN | 342 | 51.060 | −2.780 | 66.690 | 1.00 | 29.97 | B |
| ATOM | 2453 | CB | ASN | 342 | 50.689 | −2.094 | 65.369 | 1.00 | 28.90 | B |
| ATOM | 2454 | CG | ASN | 342 | 51.256 | −0.680 | 65.258 | 1.00 | 29.29 | B |
| ATOM | 2455 | OD1 | ASN | 342 | 51.568 | −0.210 | 64.161 | 1.00 | 27.68 | B |
| ATOM | 2456 | ND2 | ASN | 342 | 51.373 | 0.007 | 66.394 | 1.00 | 26.96 | B |
| ATOM | 2457 | C | ASN | 342 | 50.185 | −4.010 | 66.902 | 1.00 | 31.53 | B |
| ATOM | 2458 | O | ASN | 342 | 48.958 | −3.956 | 66.765 | 1.00 | 32.86 | B |
| ATOM | 2459 | N | LEU | 343 | 50.830 | −5.118 | 67.252 | 1.00 | 30.95 | B |
| ATOM | 2460 | CA | LEU | 343 | 50.143 | −6.387 | 67.474 | 1.00 | 30.40 | B |
| ATOM | 2461 | CB | LEU | 343 | 51.167 | −7.448 | 67.961 | 1.00 | 31.48 | B |
| ATOM | 2462 | CG | LEU | 343 | 50.755 | −8.930 | 68.109 | 1.00 | 33.60 | B |
| ATOM | 2463 | CD1 | LEU | 343 | 50.408 | −9.217 | 69.553 | 1.00 | 34.09 | B |
| ATOM | 2464 | CD2 | LEU | 343 | 49.599 | −9.270 | 67.168 | 1.00 | 31.95 | B |
| ATOM | 2465 | C | LEU | 343 | 48.945 | −6.325 | 68.422 | 1.00 | 28.19 | B |
| ATOM | 2466 | O | LEU | 343 | 47.839 | −6.698 | 68.042 | 1.00 | 29.33 | B |
| ATOM | 2467 | N | GLU | 344 | 49.145 | −5.858 | 69.647 | 1.00 | 26.96 | B |
| ATOM | 2468 | CA | GLU | 344 | 48.035 | −5.787 | 70.598 | 1.00 | 25.82 | B |
| ATOM | 2469 | CB | GLU | 344 | 48.537 | −5.276 | 71.962 | 1.00 | 27.56 | B |
| ATOM | 2470 | CG | GLU | 344 | 47.438 | −4.776 | 72.879 | 1.00 | 33.02 | B |
| ATOM | 2471 | CD | GLU | 344 | 47.884 | −4.708 | 74.329 | 1.00 | 36.74 | B |
| ATOM | 2472 | OE1 | GLU | 344 | 49.011 | −4.222 | 74.583 | 1.00 | 36.88 | B |
| ATOM | 2473 | OE2 | GLU | 344 | 47.104 | −5.138 | 75.217 | 1.00 | 38.52 | B |
| ATOM | 2474 | C | GLU | 344 | 46.843 | −4.948 | 70.122 | 1.00 | 23.12 | B |
| ATOM | 2475 | O | GLU | 344 | 45.696 | −5.357 | 70.265 | 1.00 | 22.53 | B |
| ATOM | 2476 | N | GLU | 345 | 47.102 | −3.775 | 69.564 | 1.00 | 22.13 | B |
| ATOM | 2477 | CA | GLU | 345 | 46.007 | −2.949 | 69.082 | 1.00 | 22.56 | B |
| ATOM | 2478 | CB | GLU | 345 | 46.484 | −1.487 | 68.830 | 1.00 | 23.16 | B |
| ATOM | 2479 | CG | GLU | 345 | 46.722 | −0.693 | 70.108 | 1.00 | 23.64 | B |
| ATOM | 2480 | CD | GLU | 345 | 45.440 | −0.386 | 70.872 | 1.00 | 25.85 | B |
| ATOM | 2481 | OE1 | GLU | 345 | 45.530 | 0.135 | 72.003 | 1.00 | 29.18 | B |
| ATOM | 2482 | OE2 | GLU | 345 | 44.342 | −0.653 | 70.352 | 1.00 | 25.14 | B |
| ATOM | 2483 | C | GLU | 345 | 45.422 | −3.566 | 67.808 | 1.00 | 21.03 | B |
| ATOM | 2484 | O | GLU | 345 | 44.238 | −3.398 | 67.519 | 1.00 | 20.99 | B |
| ATOM | 2485 | N | THR | 346 | 46.253 | −4.274 | 67.048 | 1.00 | 20.57 | B |
| ATOM | 2486 | CA | THR | 346 | 45.794 | −4.959 | 65.838 | 1.00 | 20.75 | B |
| ATOM | 2487 | CB | THR | 346 | 46.978 | −5.579 | 65.057 | 1.00 | 21.69 | B |
| ATOM | 2488 | OG1 | THR | 346 | 47.743 | −4.531 | 64.460 | 1.00 | 23.54 | B |
| ATOM | 2489 | CG2 | THR | 346 | 46.486 | −6.540 | 63.964 | 1.00 | 20.78 | B |
| ATOM | 2490 | C | THR | 346 | 44.825 | −6.070 | 66.269 | 1.00 | 20.06 | B |
| ATOM | 2491 | O | THR | 346 | 43.824 | −6.323 | 65.603 | 1.00 | 19.82 | B |
| ATOM | 2492 | N | LEU | 347 | 45.127 | −6.717 | 67.395 | 1.00 | 19.28 | B |
| ATOM | 2493 | CA | LEU | 347 | 44.265 | −7.771 | 67.924 | 1.00 | 20.23 | B |
| ATOM | 2494 | CB | LEU | 347 | 44.967 | −8.547 | 69.080 | 1.00 | 20.75 | B |
| ATOM | 2495 | CG | LEU | 347 | 46.123 | −9.517 | 68.681 | 1.00 | 20.74 | B |
| ATOM | 2496 | CD1 | LEU | 347 | 46.659 | −10.198 | 69.923 | 1.00 | 18.01 | B |
| ATOM | 2497 | CD2 | LEU | 347 | 45.630 | −10.563 | 67.681 | 1.00 | 19.87 | B |
| ATOM | 2498 | C | LEU | 347 | 42.950 | −7.187 | 68.426 | 1.00 | 20.24 | B |
| ATOM | 2499 | O | LEU | 347 | 41.884 | −7.735 | 68.165 | 1.00 | 20.79 | B |
| ATOM | 2500 | N | SER | 348 | 43.019 | −6.074 | 69.148 | 1.00 | 19.68 | B |
| ATOM | 2501 | CA | SER | 348 | 41.800 | −5.450 | 69.645 | 1.00 | 18.65 | B |
| ATOM | 2502 | CB | SER | 348 | 42.123 | −4.205 | 70.337 | 1.00 | 18.12 | B |
| ATOM | 2503 | OG | SER | 348 | 42.924 | −4.491 | 71.458 | 1.00 | 23.16 | B |
| ATOM | 2504 | C | SER | 348 | 40.848 | −5.161 | 68.498 | 1.00 | 18.64 | B |
| ATOM | 2505 | O | SER | 348 | 39.662 | −5.505 | 68.560 | 1.00 | 17.43 | B |
| ATOM | 2506 | N | THR | 349 | 41.377 | −4.535 | 67.447 | 1.00 | 18.49 | B |
| ATOM | 2507 | CA | THR | 349 | 40.577 | −4.195 | 66.274 | 1.00 | 20.04 | B |
| ATOM | 2508 | CB | THR | 349 | 41.440 | −3.523 | 65.189 | 1.00 | 21.24 | B |
| ATOM | 2509 | OG1 | THR | 349 | 41.774 | −2.195 | 65.607 | 1.00 | 22.77 | B |
| ATOM | 2510 | CG2 | THR | 349 | 40.692 | −3.471 | 63.848 | 1.00 | 20.74 | B |
| ATOM | 2511 | C | THR | 349 | 39.873 | −5.402 | 65.658 | 1.00 | 20.94 | B |
| ATOM | 2512 | O | THR | 349 | 38.651 | −5.399 | 65.516 | 1.00 | 19.02 | B |
| ATOM | 2513 | N | LEU | 350 | 40.645 | −6.423 | 65.280 | 1.00 | 23.75 | B |
| ATOM | 2514 | CA | LEU | 350 | 40.072 | −7.632 | 64.682 | 1.00 | 25.37 | B |
| ATOM | 2515 | CB | LEU | 350 | 41.155 | −8.728 | 64.483 | 1.00 | 24.15 | B |
| ATOM | 2516 | CG | LEU | 350 | 42.104 | −8.768 | 63.261 | 1.00 | 23.69 | B |
| ATOM | 2517 | CD1 | LEU | 350 | 41.548 | −7.931 | 62.146 | 1.00 | 24.69 | B |
| ATOM | 2518 | CD2 | LEU | 350 | 43.476 | −8.294 | 63.652 | 1.00 | 25.26 | B |
| ATOM | 2519 | C | LEU | 350 | 38.967 | −8.204 | 65.570 | 1.00 | 25.64 | B |
| ATOM | 2520 | O | LEU | 350 | 37.925 | −8.651 | 65.088 | 1.00 | 25.79 | B |
| ATOM | 2521 | N | GLU | 351 | 39.215 | −8.179 | 66.873 | 1.00 | 26.21 | B |
| ATOM | 2522 | CA | GLU | 351 | 38.280 | −8.705 | 67.859 | 1.00 | 26.22 | B |
| ATOM | 2523 | CB | GLU | 351 | 38.950 | −8.729 | 69.230 | 1.00 | 29.30 | B |
| ATOM | 2524 | CG | GLU | 351 | 38.325 | −9.722 | 70.181 | 1.00 | 35.95 | B |
| ATOM | 2525 | CD | GLU | 351 | 38.148 | −11.081 | 69.528 | 1.00 | 39.86 | B |
| ATOM | 2526 | OE1 | GLU | 351 | 39.180 | −11.726 | 69.204 | 1.00 | 39.55 | B |
| ATOM | 2527 | OE2 | GLU | 351 | 36.973 | −11.484 | 69.326 | 1.00 | 40.87 | B |
| ATOM | 2528 | C | GLU | 351 | 36.995 | −7.887 | 67.927 | 1.00 | 24.59 | B |
| ATOM | 2529 | O | GLU | 351 | 35.886 | −8.438 | 67.987 | 1.00 | 24.44 | B |
| ATOM | 2530 | N | TYR | 352 | 37.163 | −6.569 | 67.922 | 1.00 | 22.44 | B |
| ATOM | 2531 | CA | TYR | 352 | 36.058 | −5.627 | 67.973 | 1.00 | 20.05 | B |
| ATOM | 2532 | CB | TYR | 352 | 36.638 | −4.176 | 68.166 | 1.00 | 20.78 | B |
| ATOM | 2533 | CG | TYR | 352 | 35.618 | −3.065 | 68.285 | 1.00 | 19.34 | B |
| ATOM | 2534 | CD1 | TYR | 352 | 34.997 | −2.539 | 67.153 | 1.00 | 17.81 | B |
| ATOM | 2535 | CE1 | TYR | 352 | 34.062 | −1.515 | 67.258 | 1.00 | 19.71 | B |
| ATOM | 2536 | CD2 | TYR | 352 | 35.277 | −2.535 | 69.533 | 1.00 | 19.30 | B |
| ATOM | 2537 | CE2 | TYR | 352 | 34.339 | −1.507 | 69.649 | 1.00 | 17.88 | B |
| ATOM | 2538 | CZ | TYR | 352 | 33.737 | −1.003 | 68.508 | 1.00 | 19.50 | B |
| ATOM | 2539 | OH | TYR | 352 | 32.810 | 0.017 | 68.602 | 1.00 | 23.10 | B |
| ATOM | 2540 | C | TYR | 352 | 35.211 | −5.723 | 66.706 | 1.00 | 20.25 | B |
| ATOM | 2541 | O | TYR | 352 | 33.989 | −5.704 | 66.776 | 1.00 | 20.39 | B |
| ATOM | 2542 | N | ALA | 353 | 35.855 | −5.851 | 65.549 | 1.00 | 20.55 | B |
| ATOM | 2543 | CA | ALA | 353 | 35.122 | −5.941 | 64.289 | 1.00 | 23.02 | B |
| ATOM | 2544 | CB | ALA | 353 | 36.076 | −5.711 | 63.116 | 1.00 | 20.71 | B |
| ATOM | 2545 | C | ALA | 353 | 34.374 | −7.271 | 64.109 | 1.00 | 25.05 | B |
| ATOM | 2546 | O | ALA | 353 | 33.259 | −7.299 | 63.580 | 1.00 | 24.67 | B |
| ATOM | 2547 | N | HIS | 354 | 34.983 | −8.366 | 64.553 | 1.00 | 26.56 | B |
| ATOM | 2548 | CA | HIS | 354 | 34.372 | −9.682 | 64.420 | 1.00 | 29.08 | B |
| ATOM | 2549 | CB | HIS | 354 | 35.332 | −10.761 | 64.917 | 1.00 | 30.47 | B |
| ATOM | 2550 | CG | HIS | 354 | 34.916 | −12.150 | 64.547 | 1.00 | 31.52 | B |
| ATOM | 2551 | CD2 | HIS | 354 | 34.400 | −13.156 | 65.293 | 1.00 | 30.23 | B |
| ATOM | 2552 | ND1 | HIS | 354 | 34.990 | −12.629 | 63.255 | 1.00 | 32.72 | B |
| ATOM | 2553 | CE1 | HIS | 354 | 34.539 | −13.870 | 63.222 | 1.00 | 32.65 | B |
| ATOM | 2554 | NE2 | HIS | 354 | 34.175 | −14.213 | 64.445 | 1.00 | 32.59 | B |
| ATOM | 2555 | C | HIS | 354 | 33.059 | −9.754 | 65.194 | 1.00 | 30.20 | B |
| ATOM | 2556 | O | HIS | 354 | 32.075 | −10.332 | 64.722 | 1.00 | 30.57 | B |
| ATOM | 2557 | N | ARG | 355 | 33.044 | −9.177 | 66.390 | 1.00 | 31.47 | B |
| ATOM | 2558 | CA | ARG | 355 | 31.825 | −9.166 | 67.182 | 1.00 | 33.23 | B |
| ATOM | 2559 | CB | ARG | 355 | 32.064 | −8.504 | 68.551 | 1.00 | 35.96 | B |
| ATOM | 2560 | CG | ARG | 355 | 32.853 | −9.364 | 69.516 | 1.00 | 40.08 | B |
| ATOM | 2561 | CD | ARG | 355 | 33.214 | −8.625 | 70.797 | 1.00 | 43.24 | B |
| ATOM | 2562 | NE | ARG | 355 | 32.052 | −8.198 | 71.579 | 1.00 | 47.90 | B |
| ATOM | 2563 | CZ | ARG | 355 | 31.127 | −9.016 | 72.081 | 1.00 | 50.90 | B |
| ATOM | 2564 | NH1 | ARG | 355 | 31.208 | −10.329 | 71.881 | 1.00 | 50.97 | B |
| ATOM | 2565 | NH2 | ARG | 355 | 30.128 | −8.521 | 72.806 | 1.00 | 50.14 | B |
| ATOM | 2566 | C | ARG | 355 | 30.770 | −8.378 | 66.413 | 1.00 | 32.60 | B |
| ATOM | 2567 | O | ARG | 355 | 29.619 | −8.801 | 66.321 | 1.00 | 32.82 | B |
| ATOM | 2568 | N | ALA | 356 | 31.178 | −7.240 | 65.850 | 1.00 | 29.87 | B |
| ATOM | 2569 | CA | ALA | 356 | 30.266 | −6.389 | 65.096 | 1.00 | 27.94 | B |
| ATOM | 2570 | CB | ALA | 356 | 31.025 | −5.243 | 64.467 | 1.00 | 28.16 | B |
| ATOM | 2571 | C | ALA | 356 | 29.485 | −7.137 | 64.022 | 1.00 | 26.92 | B |
| ATOM | 2572 | O | ALA | 356 | 28.356 | −6.759 | 63.698 | 1.00 | 24.79 | B |
| ATOM | 2573 | N | LYS | 357 | 30.074 | −8.203 | 63.486 | 1.00 | 25.84 | B |
| ATOM | 2574 | CA | LYS | 357 | 29.416 | −8.982 | 62.438 | 1.00 | 27.17 | B |
| ATOM | 2575 | CB | LYS | 357 | 30.248 | −10.193 | 62.040 | 1.00 | 26.83 | B |
| ATOM | 2576 | CG | LYS | 357 | 31.690 | −9.905 | 61.724 | 1.00 | 28.45 | B |
| ATOM | 2577 | CD | LYS | 357 | 32.191 | −10.857 | 60.651 | 1.00 | 31.56 | B |
| ATOM | 2578 | CE | LYS | 357 | 31.933 | −12.305 | 61.008 | 1.00 | 31.36 | B |
| ATOM | 2579 | NZ | LYS | 357 | 32.361 | −13.190 | 59.908 | 1.00 | 30.37 | B |
| ATOM | 2580 | C | LYS | 357 | 28.036 | −9.483 | 62.831 | 1.00 | 27.51 | B |
| ATOM | 2581 | O | LYS | 357 | 27.173 | −9.651 | 61.974 | 1.00 | 27.57 | B |
| ATOM | 2582 | N | ASN | 358 | 27.829 | −9.728 | 64.121 | 1.00 | 28.92 | B |
| ATOM | 2583 | CA | ASN | 358 | 26.546 | −10.234 | 64.597 | 1.00 | 30.60 | B |
| ATOM | 2584 | CB | ASN | 358 | 26.741 | −11.024 | 65.911 | 1.00 | 31.34 | B |
| ATOM | 2585 | CG | ASN | 358 | 27.527 | −12.311 | 65.709 | 1.00 | 33.50 | B |
| ATOM | 2586 | OD1 | ASN | 358 | 28.750 | −12.292 | 65.537 | 1.00 | 34.98 | B |
| ATOM | 2587 | ND2 | ASN | 358 | 26.823 | −13.439 | 65.716 | 1.00 | 33.36 | B |
| ATOM | 2588 | C | ASN | 358 | 25.426 | −9.207 | 64.788 | 1.00 | 30.89 | B |
| ATOM | 2589 | O | ASN | 358 | 24.367 | −9.547 | 65.302 | 1.00 | 32.42 | B |
| ATOM | 2590 | N | ILE | 359 | 25.642 | −7.961 | 64.381 | 1.00 | 31.36 | B |
| ATOM | 2591 | CA | ILE | 359 | 24.607 | −6.943 | 64.530 | 1.00 | 31.09 | B |
| ATOM | 2592 | CB | ILE | 359 | 25.185 | −5.505 | 64.454 | 1.00 | 30.83 | B |
| ATOM | 2593 | CG2 | ILE | 359 | 24.060 | −4.493 | 64.496 | 1.00 | 28.14 | B |
| ATOM | 2594 | CG1 | ILE | 359 | 26.144 | −5.246 | 65.629 | 1.00 | 29.88 | B |
| ATOM | 2595 | CD1 | ILE | 359 | 27.028 | −4.031 | 65.421 | 1.00 | 29.12 | B |
| ATOM | 2596 | C | ILE | 359 | 23.583 | −7.110 | 63.416 | 1.00 | 32.70 | B |
| ATOM | 2597 | O | ILE | 359 | 23.938 | −7.293 | 62.250 | 1.00 | 31.89 | B |
| ATOM | 2598 | N | LEU | 360 | 22.312 | −7.045 | 63.795 | 1.00 | 34.93 | B |
| ATOM | 2599 | CA | LEU | 360 | 21.195 | −7.185 | 62.869 | 1.00 | 37.63 | B |
| ATOM | 2600 | CB | LEU | 360 | 20.056 | −7.993 | 63.544 | 1.00 | 39.00 | B |
| ATOM | 2601 | CG | LEU | 360 | 18.581 | −7.590 | 63.189 | 1.00 | 41.16 | B |
| ATOM | 2602 | CD1 | LEU | 360 | 18.283 | −7.917 | 61.728 | 1.00 | 42.20 | B |
| ATOM | 2603 | CD2 | LEU | 360 | 17.599 | −8.315 | 64.118 | 1.00 | 41.50 | B |
| ATOM | 2604 | C | LEU | 360 | 20.672 | −5.814 | 62.475 | 1.00 | 38.26 | B |
| ATOM | 2605 | O | LEU | 360 | 20.356 | −5.003 | 63.343 | 1.00 | 38.46 | B |
| ATOM | 2606 | N | ASN | 361 | 20.580 | −5.565 | 61.171 | 1.00 | 39.80 | B |
| ATOM | 2607 | CA | ASN | 361 | 20.079 | −4.295 | 60.656 | 1.00 | 41.76 | B |
| ATOM | 2608 | CB | ASN | 361 | 21.133 | −3.606 | 59.822 | 1.00 | 42.66 | B |
| ATOM | 2609 | CG | ASN | 361 | 22.088 | −2.772 | 60.657 | 1.00 | 44.51 | B |
| ATOM | 2610 | OD1 | ASN | 361 | 22.791 | −3.289 | 61.528 | 1.00 | 45.27 | B |
| ATOM | 2611 | ND2 | ASN | 361 | 22.117 | −1.467 | 60.394 | 1.00 | 45.23 | B |
| ATOM | 2612 | C | ASN | 361 | 18.825 | −4.481 | 59.812 | 1.00 | 44.12 | B |
| ATOM | 2613 | O | ASN | 361 | 18.478 | −5.604 | 59.438 | 1.00 | 45.59 | B |
| ATOM | 2614 | N | LYS | 362 | 18.160 | −3.366 | 59.514 | 1.00 | 45.40 | B |
| ATOM | 2615 | CA | LYS | 362 | 16.931 | −3.332 | 58.716 | 1.00 | 45.80 | B |
| ATOM | 2616 | CB | LYS | 362 | 17.226 | −3.756 | 57.260 | 1.00 | 45.62 | B |
| ATOM | 2617 | CG | LYS | 362 | 17.222 | −2.619 | 56.240 | 1.00 | 45.92 | B |
| ATOM | 2618 | CD | LYS | 362 | 15.832 | −2.001 | 56.093 | 1.00 | 45.58 | B |
| ATOM | 2619 | CE | LYS | 362 | 15.739 | −1.104 | 54.862 | 1.00 | 43.34 | B |
| ATOM | 2620 | NZ | LYS | 362 | 14.456 | −0.345 | 54.818 | 1.00 | 42.49 | B |
| ATOM | 2621 | C | LYS | 362 | 15.823 | −4.213 | 59.292 | 1.00 | 47.03 | B |
| ATOM | 2622 | O | LYS | 362 | 15.150 | −4.897 | 58.492 | 1.00 | 48.78 | B |
| ATOM | 2623 | OXT | LYS | 362 | 15.624 | −4.198 | 60.526 | 1.00 | 47.26 | B |
| ATOM | 2624 | MG | MG | 2602 | 43.330 | 10.372 | 60.103 | 1.00 | 26.54 | |
| ATOM | 2625 | PB | ADP | 2600 | 44.452 | 7.135 | 60.400 | 1.00 | 17.43 | ADP |
| ATOM | 2626 | O1B | ADP | 2600 | 44.951 | 7.845 | 61.612 | 1.00 | 18.86 | ADP |
| ATOM | 2627 | O2B | ADP | 2600 | 44.008 | 5.637 | 60.747 | 1.00 | 22.98 | ADP |
| ATOM | 2628 | O3B | ADP | 2600 | 43.299 | 7.848 | 59.790 | 1.00 | 19.76 | ADP |
| ATOM | 2629 | PA | ADP | 2600 | 45.880 | 7.608 | 57.967 | 1.00 | 24.97 | ADP |
| ATOM | 2630 | O1A | ADP | 2600 | 44.906 | 7.153 | 56.989 | 1.00 | 27.54 | ADP |
| ATOM | 2631 | O2A | ADP | 2600 | 45.805 | 9.067 | 58.061 | 1.00 | 29.40 | ADP |
| ATOM | 2632 | O3A | ADP | 2600 | 45.606 | 6.967 | 59.369 | 1.00 | 22.28 | ADP |
| ATOM | 2633 | O5* | ADP | 2600 | 47.347 | 7.314 | 57.518 | 1.00 | 28.31 | ADP |
| ATOM | 2634 | C5* | ADP | 2600 | 48.422 | 6.620 | 58.144 | 1.00 | 30.71 | ADP |
| ATOM | 2635 | C4* | ADP | 2600 | 49.601 | 6.747 | 57.103 | 1.00 | 33.98 | ADP |
| ATOM | 2636 | O4* | ADP | 2600 | 49.664 | 5.485 | 56.457 | 1.00 | 33.98 | ADP |
| ATOM | 2637 | C3* | ADP | 2600 | 49.383 | 7.792 | 55.972 | 1.00 | 32.52 | ADP |
| ATOM | 2638 | O3* | ADP | 2600 | 50.518 | 8.657 | 55.838 | 1.00 | 36.94 | ADP |
| ATOM | 2639 | C2* | ADP | 2600 | 49.106 | 7.017 | 54.682 | 1.00 | 35.49 | ADP |
| ATOM | 2640 | O2* | ADP | 2600 | 49.782 | 7.556 | 53.522 | 1.00 | 38.23 | ADP |
| ATOM | 2641 | C1* | ADP | 2600 | 49.483 | 5.577 | 55.026 | 1.00 | 35.20 | ADP |
| ATOM | 2642 | N9 | ADP | 2600 | 48.437 | 4.548 | 54.689 | 1.00 | 33.78 | ADP |
| ATOM | 2643 | C8 | ADP | 2600 | 47.512 | 4.099 | 55.567 | 1.00 | 34.18 | ADP |
| ATOM | 2644 | N7 | ADP | 2600 | 46.745 | 3.202 | 55.003 | 1.00 | 36.36 | ADP |
| ATOM | 2645 | C5 | ADP | 2600 | 47.137 | 3.045 | 53.768 | 1.00 | 36.94 | ADP |
| ATOM | 2646 | C6 | ADP | 2600 | 46.721 | 2.241 | 52.700 | 1.00 | 37.31 | ADP |
| ATOM | 2647 | N6 | ADP | 2600 | 45.687 | 1.403 | 52.874 | 1.00 | 37.72 | ADP |
| ATOM | 2648 | N1 | ADP | 2600 | 47.381 | 2.320 | 51.471 | 1.00 | 37.39 | ADP |
| ATOM | 2649 | C2 | ADP | 2600 | 48.446 | 3.171 | 51.268 | 1.00 | 37.76 | ADP |
| ATOM | 2650 | N3 | ADP | 2600 | 48.859 | 3.957 | 52.311 | 1.00 | 35.88 | ADP |
| ATOM | 2651 | C4 | ADP | 2600 | 48.245 | 3.925 | 53.548 | 1.00 | 35.51 | ADP |
| ATOM | 2652 | C1 | 1-7 | 1 | 37.929 | 17.272 | 54.077 | 1.00 | 38.43 | 1-7 |
| ATOM | 2653 | C2 | 1-7 | 1 | 38.932 | 17.045 | 53.074 | 1.00 | 38.52 | 1-7 |
| ATOM | 2654 | C3 | 1-7 | 1 | 38.735 | 15.932 | 52.163 | 1.00 | 39.96 | 1-7 |
| ATOM | 2655 | C4 | 1-7 | 1 | 37.528 | 15.091 | 52.280 | 1.00 | 39.17 | 1-7 |
| ATOM | 2656 | C5 | 1-7 | 1 | 36.503 | 15.314 | 53.268 | 1.00 | 37.92 | 1-7 |
| ATOM | 2657 | C6 | 1-7 | 1 | 36.737 | 16.421 | 54.166 | 1.00 | 39.95 | 1-7 |
| ATOM | 2658 | C11 | 1-7 | 1 | 39.781 | 15.680 | 51.154 | 1.00 | 38.83 | 1-7 |
| ATOM | 2659 | N12 | 1-7 | 1 | 40.860 | 16.465 | 50.816 | 1.00 | 41.41 | 1-7 |
| ATOM | 2660 | N13 | 1-7 | 1 | 41.632 | 15.978 | 49.912 | 1.00 | 42.37 | 1-7 |
| ATOM | 2661 | C14 | 1-7 | 1 | 41.128 | 14.690 | 49.355 | 1.00 | 40.44 | 1-7 |
| ATOM | 2662 | C15 | 1-7 | 1 | 40.183 | 14.416 | 50.455 | 1.00 | 39.39 | 1-7 |
| ATOM | 2663 | C18 | 1-7 | 1 | 41.056 | 14.226 | 47.951 | 1.00 | 36.95 | 1-7 |
| ATOM | 2664 | C20 | 1-7 | 1 | 42.809 | 16.554 | 49.520 | 1.00 | 43.23 | 1-7 |
| ATOM | 2665 | C21 | 1-7 | 1 | 43.706 | 15.596 | 48.761 | 1.00 | 42.51 | 1-7 |
| ATOM | 2666 | O25 | 1-7 | 1 | 43.145 | 17.720 | 49.767 | 1.00 | 44.94 | 1-7 |
| ATOM | 2667 | C26 | 1-7 | 1 | 40.067 | 14.828 | 47.075 | 1.00 | 35.46 | 1-7 |
| ATOM | 2668 | C27 | 1-7 | 1 | 40.008 | 14.513 | 45.661 | 1.00 | 35.09 | 1-7 |
| ATOM | 2669 | C28 | 1-7 | 1 | 40.989 | 13.573 | 45.157 | 1.00 | 34.04 | 1-7 |
| ATOM | 2670 | C29 | 1-7 | 1 | 41.984 | 12.977 | 46.048 | 1.00 | 34.13 | 1-7 |
| ATOM | 2671 | C30 | 1-7 | 1 | 42.012 | 13.263 | 47.467 | 1.00 | 34.81 | 1-7 |
| ATOM | 2672 | CL35 | 1-7 | 1 | 37.356 | 13.776 | 51.201 | 1.00 | 40.06 | 1-7 |
| ATOM | 2673 | O36 | 1-7 | 1 | 42.983 | 12.166 | 45.535 | 1.00 | 32.08 | 1-7 |
| ATOM | 2674 | O | HOH | 2 | 38.525 | 10.810 | 62.766 | 1.00 | 2.98 | S |
| ATOM | 2675 | O | HOH | 3 | 23.222 | 11.589 | 60.100 | 1.00 | 22.29 | S |
| ATOM | 2676 | O | HOH | 4 | 41.960 | 12.208 | 60.870 | 1.00 | 9.69 | S |
| ATOM | 2677 | O | HOH | 5 | 50.029 | −4.994 | 63.682 | 1.00 | 18.21 | S |
| ATOM | 2678 | O | HOH | 8 | 28.413 | 21.060 | 56.800 | 1.00 | 20.56 | S |
| ATOM | 2679 | O | HOH | 9 | 31.397 | 6.826 | 80.114 | 1.00 | 18.48 | S |
| ATOM | 2680 | O | HOH | 10 | 38.337 | 3.375 | 65.490 | 1.00 | 21.12 | S |
| ATOM | 2681 | O | HOH | 13 | 45.628 | 22.010 | 69.140 | 1.00 | 9.64 | S |
| ATOM | 2682 | O | HOH | 14 | 48.257 | 14.330 | 41.733 | 1.00 | 18.62 | S |
| ATOM | 2683 | O | HOH | 15 | 41.014 | 5.558 | 71.890 | 1.00 | 28.07 | S |
| ATOM | 2684 | O | HOH | 16 | 27.936 | 20.868 | 70.581 | 1.00 | 22.56 | S |
| ATOM | 2685 | O | HOH | 17 | 43.663 | −1.056 | 64.226 | 1.00 | 13.66 | S |
| ATOM | 2686 | O | HOH | 18 | 43.194 | 8.354 | 64.240 | 1.00 | 19.73 | S |
| ATOM | 2687 | O | HOH | 20 | 54.924 | 6.098 | 49.933 | 1.00 | 32.18 | S |
| ATOM | 2688 | O | HOH | 22 | 31.350 | 4.322 | 82.668 | 1.00 | 37.14 | S |
| ATOM | 2689 | O | HOH | 27 | 45.521 | −1.603 | 51.520 | 1.00 | 20.22 | S |
| ATOM | 2690 | O | HOH | 28 | 53.208 | 11.559 | 41.772 | 1.00 | 42.11 | S |
| ATOM | 2691 | O | HOH | 31 | 27.994 | 6.504 | 79.871 | 1.00 | 18.94 | S |
| ATOM | 2692 | O | HOH | 33 | 49.291 | −7.879 | 50.486 | 1.00 | 35.78 | S |
| ATOM | 2693 | O | HOH | 34 | 18.468 | 12.203 | 33.372 | 1.00 | 19.62 | S |
| ATOM | 2694 | O | HOH | 35 | 53.496 | −17.951 | 61.642 | 1.00 | 35.98 | S |
| ATOM | 2695 | O | HOH | 36 | 45.680 | 3.185 | 45.465 | 1.00 | 19.30 | S |
| ATOM | 2696 | O | HOH | 38 | 42.176 | −0.846 | 72.113 | 1.00 | 14.70 | S |
| ATOM | 2697 | O | HOH | 39 | 51.304 | 5.232 | 60.441 | 1.00 | 24.96 | S |
| ATOM | 2698 | O | HOH | 40 | 34.806 | 13.087 | 70.806 | 1.00 | 32.37 | S |
| ATOM | 2699 | O | HOH | 41 | 19.156 | 14.294 | 56.441 | 1.00 | 28.63 | S |
| ATOM | 2700 | O | HOH | 46 | 44.126 | 0.351 | 55.876 | 1.00 | 28.55 | S |
| ATOM | 2701 | O | HOH | 47 | 20.432 | 7.836 | 62.530 | 1.00 | 16.12 | S |
| ATOM | 2702 | O | HOH | 48 | 31.643 | 24.934 | 63.575 | 1.00 | 31.65 | S |
| ATOM | 2703 | O | HOH | 50 | 45.290 | 17.359 | 64.325 | 1.00 | 15.86 | S |
| ATOM | 2704 | O | HOH | 53 | 41.790 | 5.942 | 40.546 | 1.00 | 28.37 | S |
| ATOM | 2705 | O | HOH | 54 | 38.452 | 4.419 | 47.214 | 1.00 | 14.56 | S |
| ATOM | 2706 | O | HOH | 55 | 52.009 | 4.613 | 57.096 | 1.00 | 35.87 | S |
| ATOM | 2707 | O | HOH | 57 | 51.429 | 6.864 | 39.244 | 1.00 | 27.91 | S |
| ATOM | 2708 | O | HOH | 58 | 22.685 | 19.136 | 43.047 | 1.00 | 29.36 | S |
| ATOM | 2709 | O | HOH | 61 | 39.044 | 12.519 | 58.483 | 1.00 | 28.94 | S |
| ATOM | 2710 | O | HOH | 67 | 45.314 | −7.264 | 72.406 | 1.00 | 17.23 | S |
| ATOM | 2711 | O | HOH | 69 | 46.768 | −2.040 | 64.134 | 1.00 | 23.58 | S |
| ATOM | 2712 | O | HOH | 71 | 45.298 | 18.821 | 48.751 | 1.00 | 30.98 | S |
| ATOM | 2713 | O | HOH | 79 | 45.903 | 11.457 | 63.308 | 1.00 | 21.87 | S |
| ATOM | 2714 | O | HOH | 83 | 29.506 | −5.557 | 49.394 | 1.00 | 32.50 | S |
| ATOM | 2715 | O | HOH | 86 | 28.178 | 4.602 | 77.098 | 1.00 | 29.04 | S |
| ATOM | 2716 | O | HOH | 89 | 55.210 | −16.662 | 58.167 | 1.00 | 35.61 | S |
| ATOM | 2717 | O | HOH | 91 | 37.135 | 0.846 | 70.878 | 1.00 | 20.52 | S |
| ATOM | 2718 | O | HOH | 93 | 17.438 | 19.816 | 52.756 | 1.00 | 35.47 | S |
| ATOM | 2719 | O | HOH | 94 | 29.881 | 3.798 | 41.417 | 1.00 | 42.97 | S |
| ATOM | 2720 | O | HOH | 98 | 39.190 | 3.892 | 49.946 | 1.00 | 13.01 | S |
| ATOM | 2721 | O | HOH | 100 | 41.671 | 15.312 | 56.323 | 1.00 | 31.21 | S |
| ATOM | 2722 | O | HOH | 101 | 52.876 | 0.835 | 68.812 | 1.00 | 32.79 | S |
| ATOM | 2723 | O | HOH | 105 | 37.722 | 2.513 | 73.490 | 1.00 | 36.02 | S |
| ATOM | 2724 | O | HOH | 109 | 27.450 | 25.927 | 61.040 | 1.00 | 42.15 | S |
| ATOM | 2725 | O | HOH | 111 | 39.804 | 17.000 | 76.527 | 1.00 | 40.03 | S |
| ATOM | 2726 | O | HOH | 117 | 2.532 | 6.263 | 36.270 | 1.00 | 22.77 | S |
| ATOM | 2727 | O | HOH | 119 | 43.756 | 2.932 | 43.574 | 1.00 | 30.63 | S |
| ATOM | 2728 | O | HOH | 124 | 41.324 | 9.248 | 61.513 | 1.00 | 50.60 | S |
| ATOM | 2729 | O | HOH | 128 | 45.349 | 21.055 | 46.092 | 1.00 | 34.28 | S |
| ATOM | 2730 | O | HOH | 129 | 47.480 | 9.402 | 61.725 | 1.00 | 20.53 | S |
| ATOM | 2731 | O | HOH | 130 | 27.022 | 14.663 | 58.188 | 1.00 | 21.56 | S |
| ATOM | 2732 | O | HOH | 131 | 38.009 | 11.637 | 34.970 | 1.00 | 36.04 | S |
| ATOM | 2733 | O | HOH | 135 | 21.462 | 18.078 | 39.253 | 1.00 | 49.42 | S |
| ATOM | 2734 | O | HOH | 136 | 50.206 | −0.381 | 68.977 | 1.00 | 28.73 | S |
| ATOM | 2735 | O | HOH | 142 | 43.209 | 19.312 | 57.176 | 1.00 | 32.90 | S |
| ATOM | 2736 | O | HOH | 144 | 27.420 | −13.840 | 56.585 | 1.00 | 40.61 | S |
| ATOM | 2737 | O | HOH | 145 | 56.085 | 3.298 | 61.538 | 1.00 | 27.46 | S |
| ATOM | 2738 | O | HOH | 148 | 45.044 | 22.181 | 54.899 | 1.00 | 33.67 | S |
| ATOM | 2739 | O | HOH | 149 | 47.168 | 9.785 | 68.295 | 1.00 | 32.20 | S |
| ATOM | 2740 | O | HOH | 150 | 35.221 | 13.107 | 56.556 | 1.00 | 39.71 | S |
| ATOM | 2741 | O | HOH | 156 | 19.494 | 13.147 | 35.697 | 1.00 | 37.79 | S |
| ATOM | 2742 | O | HOH | 158 | 35.348 | 1.853 | 79.606 | 1.00 | 35.97 | S |
| ATOM | 2743 | O | HOH | 160 | 44.086 | −3.335 | 73.582 | 1.00 | 28.68 | S |
| ATOM | 2744 | O | HOH | 163 | 22.716 | 28.692 | 55.723 | 1.00 | 38.12 | S |
| ATOM | 2745 | O | HOH | 164 | 29.077 | 26.837 | 62.948 | 1.00 | 37.04 | S |
| END | | | | | | | | | | |

TABLE 3
REMARK refinement resolution: 50.0-2.5 A
REMARK final r = 0.2461 free_r = 0.3007
REMARK rmsd bonds = 0.007673 rmsd angles = 1.23268
REMARK sg = P2(1)2(1)2(1) a = 68.9 b = 79.4 c = 158.8 alpha = 90.
beta = 90. gamma = 90.
REMARK FILENAME = “Compound 2-7_3pb.pdb”

| | | | | | | | | | | |
|---|---|---|---|---|---|---|---|---|---|---|
| ATOM | 1 | CB | LYS | 17 | 24.357 | −12.099 | 59.933 | 1.00 | 58.09 | B |
| ATOM | 2 | CG | LYS | 17 | 23.017 | −12.631 | 59.411 | 1.00 | 60.84 | B |
| ATOM | 3 | CD | LYS | 17 | 22.865 | −12.482 | 57.896 | 1.00 | 62.11 | B |
| ATOM | 4 | CE | LYS | 17 | 23.604 | −13.578 | 57.123 | 1.00 | 63.01 | B |
| ATOM | 5 | NZ | LYS | 17 | 25.089 | −13.550 | 57.289 | 1.00 | 63.35 | B |
| ATOM | 6 | C | LYS | 17 | 24.262 | −9.737 | 59.096 | 1.00 | 54.65 | B |
| ATOM | 7 | O | LYS | 17 | 25.150 | −9.723 | 58.262 | 1.00 | 53.83 | B |
| ATOM | 8 | N | LYS | 17 | 23.253 | −10.341 | 61.285 | 1.00 | 56.25 | B |
| ATOM | 9 | CA | LYS | 17 | 24.364 | −10.617 | 60.333 | 1.00 | 55.82 | B |
| ATOM | 10 | N | ASN | 18 | 23.168 | −8.993 | 58.994 | 1.00 | 53.57 | B |
| ATOM | 11 | CA | ASN | 18 | 22.956 | −8.115 | 57.857 | 1.00 | 52.96 | B |
| ATOM | 12 | CB | ASN | 18 | 21.634 | −7.362 | 58.018 | 1.00 | 55.67 | B |
| ATOM | 13 | CG | ASN | 18 | 20.433 | −8.197 | 57.613 | 1.00 | 58.59 | B |
| ATOM | 14 | OD1 | ASN | 18 | 20.173 | −9.261 | 58.187 | 1.00 | 59.98 | B |
| ATOM | 15 | ND2 | ASN | 18 | 19.688 | −7.717 | 56.621 | 1.00 | 58.01 | B |
| ATOM | 16 | C | ASN | 18 | 24.093 | −7.115 | 57.635 | 1.00 | 51.27 | B |
| ATOM | 17 | O | ASN | 18 | 24.391 | −6.754 | 56.495 | 1.00 | 52.49 | B |
| ATOM | 18 | N | ILE | 19 | 24.723 | −6.665 | 58.716 | 1.00 | 47.11 | B |
| ATOM | 19 | CA | ILE | 19 | 25.811 | −5.698 | 58.613 | 1.00 | 42.06 | B |
| ATOM | 20 | CB | ILE | 19 | 26.192 | −5.152 | 60.004 | 1.00 | 42.31 | B |
| ATOM | 21 | CG2 | ILE | 19 | 26.598 | −6.295 | 60.917 | 1.00 | 43.22 | B |
| ATOM | 22 | CG1 | ILE | 19 | 27.343 | −4.159 | 59.881 | 1.00 | 41.90 | B |
| ATOM | 23 | CD1 | ILE | 19 | 27.762 | −3.556 | 61.193 | 1.00 | 43.78 | B |
| ATOM | 24 | C | ILE | 19 | 27.054 | −6.300 | 57.958 | 1.00 | 38.26 | B |
| ATOM | 25 | O | ILE | 19 | 27.480 | −7.376 | 58.312 | 1.00 | 38.23 | B |
| ATOM | 26 | N | GLN | 20 | 27.627 | −5.577 | 56.999 | 1.00 | 34.90 | B |
| ATOM | 27 | CA | GLN | 20 | 28.820 | −6.021 | 56.279 | 1.00 | 30.15 | B |
| ATOM | 28 | CB | GLN | 20 | 28.778 | −5.516 | 54.838 | 1.00 | 27.85 | B |
| ATOM | 29 | CG | GLN | 20 | 30.034 | −5.802 | 54.038 | 1.00 | 26.74 | B |
| ATOM | 30 | CD | GLN | 20 | 29.987 | −5.186 | 52.643 | 1.00 | 27.60 | B |
| ATOM | 31 | OE1 | GLN | 20 | 30.137 | −3.984 | 52.484 | 1.00 | 29.30 | B |
| ATOM | 32 | NE2 | GLN | 20 | 29.774 | −6.017 | 51.632 | 1.00 | 26.15 | B |
| ATOM | 33 | C | GLN | 20 | 30.091 | −5.507 | 56.949 | 1.00 | 29.28 | B |
| ATOM | 34 | O | GLN | 20 | 30.186 | −4.346 | 57.290 | 1.00 | 29.19 | B |
| ATOM | 35 | N | VAL | 21 | 31.075 | −6.379 | 57.127 | 1.00 | 27.08 | B |
| ATOM | 36 | CA | VAL | 21 | 32.325 | −5.975 | 57.754 | 1.00 | 24.84 | B |
| ATOM | 37 | CB | VAL | 21 | 32.448 | −6.546 | 59.180 | 1.00 | 24.84 | B |
| ATOM | 38 | CG1 | VAL | 21 | 33.766 | −6.123 | 59.804 | 1.00 | 23.30 | B |
| ATOM | 39 | CG2 | VAL | 21 | 31.274 | −6.078 | 60.033 | 1.00 | 24.09 | B |
| ATOM | 40 | C | VAL | 21 | 33.524 | −6.439 | 56.938 | 1.00 | 24.57 | B |
| ATOM | 41 | O | VAL | 21 | 33.677 | −7.608 | 56.687 | 1.00 | 24.54 | B |
| ATOM | 42 | N | VAL | 22 | 34.370 | −5.496 | 56.531 | 1.00 | 25.16 | B |
| ATOM | 43 | CA | VAL | 22 | 35.558 | −5.818 | 55.753 | 1.00 | 24.51 | B |
| ATOM | 44 | CB | VAL | 22 | 35.493 | −5.171 | 54.356 | 1.00 | 25.74 | B |
| ATOM | 45 | CG1 | VAL | 22 | 34.274 | −5.694 | 53.602 | 1.00 | 23.07 | B |
| ATOM | 46 | CG2 | VAL | 22 | 35.428 | −3.648 | 54.488 | 1.00 | 26.13 | B |
| ATOM | 47 | C | VAL | 22 | 36.825 | −5.350 | 56.464 | 1.00 | 24.25 | B |
| ATOM | 48 | O | VAL | 22 | 36.769 | −4.532 | 57.376 | 1.00 | 25.41 | B |
| ATOM | 49 | N | VAL | 23 | 37.964 | −5.889 | 56.047 | 1.00 | 21.62 | B |
| ATOM | 50 | CA | VAL | 23 | 39.249 | −5.541 | 56.640 | 1.00 | 20.21 | B |
| ATOM | 51 | CB | VAL | 23 | 39.875 | −6.749 | 57.398 | 1.00 | 19.81 | B |
| ATOM | 52 | CG1 | VAL | 23 | 41.246 | −6.386 | 57.920 | 1.00 | 17.77 | B |
| ATOM | 53 | CG2 | VAL | 23 | 38.980 | −7.164 | 58.552 | 1.00 | 19.57 | B |
| ATOM | 54 | C | VAL | 23 | 40.224 | −5.069 | 55.565 | 1.00 | 20.21 | B |
| ATOM | 55 | O | VAL | 23 | 40.231 | −5.587 | 54.453 | 1.00 | 18.34 | B |
| ATOM | 56 | N | ARG | 24 | 41.026 | −4.063 | 55.908 | 1.00 | 20.97 | B |
| ATOM | 57 | CA | ARG | 24 | 42.012 | −3.508 | 54.987 | 1.00 | 23.76 | B |
| ATOM | 58 | CB | ARG | 24 | 41.493 | −2.221 | 54.341 | 1.00 | 19.71 | B |
| ATOM | 59 | CG | ARG | 24 | 42.364 | −1.729 | 53.201 | 1.00 | 19.19 | B |
| ATOM | 60 | CD | ARG | 24 | 42.064 | −0.294 | 52.784 | 1.00 | 17.94 | B |
| ATOM | 61 | NE | ARG | 24 | 42.664 | 0.010 | 51.487 | 1.00 | 16.57 | B |
| ATOM | 62 | CZ | ARG | 24 | 42.479 | 1.134 | 50.801 | 1.00 | 18.90 | B |
| ATOM | 63 | NH1 | ARG | 24 | 41.704 | 2.100 | 51.281 | 1.00 | 16.81 | B |
| ATOM | 64 | NH2 | ARG | 24 | 43.057 | 1.275 | 49.615 | 1.00 | 16.05 | B |
| ATOM | 65 | C | ARG | 24 | 43.304 | −3.210 | 55.736 | 1.00 | 27.05 | B |
| ATOM | 66 | O | ARG | 24 | 43.313 | −2.442 | 56.712 | 1.00 | 27.85 | B |
| ATOM | 67 | N | CYS | 25 | 44.392 | −3.820 | 55.274 | 1.00 | 29.51 | B |
| ATOM | 68 | CA | CYS | 25 | 45.699 | −3.637 | 55.890 | 1.00 | 32.32 | B |
| ATOM | 69 | CB | CYS | 25 | 46.410 | −4.991 | 56.027 | 1.00 | 30.86 | B |
| ATOM | 70 | SG | CYS | 25 | 48.111 | −4.890 | 56.627 | 1.00 | 32.54 | B |
| ATOM | 71 | C | CYS | 25 | 46.545 | −2.696 | 55.045 | 1.00 | 33.84 | B |
| ATOM | 72 | O | CYS | 25 | 46.587 | −2.820 | 53.831 | 1.00 | 35.92 | B |
| ATOM | 73 | N | ARG | 26 | 47.218 | −1.754 | 55.694 | 1.00 | 34.94 | B |
| ATOM | 74 | CA | ARG | 26 | 48.053 | −0.807 | 54.967 | 1.00 | 37.11 | B |
| ATOM | 75 | CB | ARG | 26 | 48.130 | 0.526 | 55.723 | 1.00 | 37.77 | B |
| ATOM | 76 | CG | ARG | 26 | 48.388 | 0.384 | 57.222 | 1.00 | 37.85 | B |
| ATOM | 77 | CD | ARG | 26 | 49.107 | 1.591 | 57.802 | 1.00 | 36.08 | B |
| ATOM | 78 | NE | ARG | 26 | 50.554 | 1.433 | 57.704 | 1.00 | 35.38 | B |
| ATOM | 79 | CZ | ARG | 26 | 51.379 | 1.390 | 58.747 | 1.00 | 35.56 | B |
| ATOM | 80 | NH1 | ARG | 26 | 50.910 | 1.502 | 59.982 | 1.00 | 32.33 | B |
| ATOM | 81 | NH2 | ARG | 26 | 52.677 | 1.209 | 58.551 | 1.00 | 37.10 | B |
| ATOM | 82 | C | ARG | 26 | 49.463 | −1.341 | 54.751 | 1.00 | 38.55 | B |
| ATOM | 83 | O | ARG | 26 | 49.917 | −2.224 | 55.460 | 1.00 | 38.07 | B |
| ATOM | 84 | N | PRO | 27 | 50.170 | −0.806 | 53.752 | 1.00 | 40.05 | B |
| ATOM | 85 | CD | PRO | 27 | 49.674 | 0.092 | 52.693 | 1.00 | 41.26 | B |
| ATOM | 86 | CA | PRO | 27 | 51.536 | −1.244 | 53.467 | 1.00 | 42.07 | B |
| ATOM | 87 | CB | PRO | 27 | 51.734 | −0.805 | 52.021 | 1.00 | 42.46 | B |
| ATOM | 88 | CG | PRO | 27 | 50.945 | 0.468 | 51.961 | 1.00 | 41.54 | B |
| ATOM | 89 | C | PRO | 27 | 52.508 | −0.555 | 54.418 | 1.00 | 43.29 | B |
| ATOM | 90 | O | PRO | 27 | 52.115 | 0.329 | 55.170 | 1.00 | 43.49 | B |
| ATOM | 91 | N | PHE | 28 | 53.773 | −0.968 | 54.380 | 1.00 | 45.76 | B |
| ATOM | 92 | CA | PHE | 28 | 54.807 | −0.381 | 55.233 | 1.00 | 47.49 | B |
| ATOM | 93 | CB | PHE | 28 | 56.045 | −1.290 | 55.308 | 1.00 | 46.30 | B |
| ATOM | 94 | CG | PHE | 28 | 55.770 | −2.659 | 55.861 | 1.00 | 45.96 | B |
| ATOM | 95 | CD1 | PHE | 28 | 55.424 | −3.709 | 55.015 | 1.00 | 45.49 | B |
| ATOM | 96 | CD2 | PHE | 28 | 55.849 | −2.899 | 57.230 | 1.00 | 45.19 | B |
| ATOM | 97 | CE1 | PHE | 28 | 55.162 | −4.976 | 55.526 | 1.00 | 44.86 | B |
| ATOM | 98 | CE2 | PHE | 28 | 55.588 | −4.165 | 57.751 | 1.00 | 44.92 | B |
| ATOM | 99 | CZ | PHE | 28 | 55.244 | −5.204 | 56.897 | 1.00 | 43.96 | B |
| ATOM | 100 | C | PHE | 28 | 55.240 | 0.974 | 54.686 | 1.00 | 49.68 | B |
| ATOM | 101 | O | PHE | 28 | 55.458 | 1.127 | 53.484 | 1.00 | 50.76 | B |
| ATOM | 102 | N | ASN | 29 | 55.369 | 1.955 | 55.572 | 1.00 | 51.78 | B |
| ATOM | 103 | CA | ASN | 29 | 55.791 | 3.289 | 55.164 | 1.00 | 53.98 | B |
| ATOM | 104 | CB | ASN | 29 | 55.477 | 4.303 | 56.268 | 1.00 | 52.37 | B |
| ATOM | 105 | CG | ASN | 29 | 55.889 | 3.818 | 57.647 | 1.00 | 51.95 | B |
| ATOM | 106 | OD1 | ASN | 29 | 57.068 | 3.614 | 57.918 | 1.00 | 51.68 | B |
| ATOM | 107 | ND2 | ASN | 29 | 54.909 | 3.633 | 58.526 | 1.00 | 50.23 | B |
| ATOM | 108 | C | ASN | 29 | 57.285 | 3.275 | 54.841 | 1.00 | 56.89 | B |
| ATOM | 109 | O | ASN | 29 | 57.973 | 2.293 | 55.111 | 1.00 | 57.68 | B |
| ATOM | 110 | N | LEU | 30 | 57.779 | 4.361 | 54.257 | 1.00 | 59.05 | B |
| ATOM | 111 | CA | LEU | 30 | 59.185 | 4.452 | 53.882 | 1.00 | 60.93 | B |
| ATOM | 112 | CB | LEU | 30 | 59.466 | 5.837 | 53.293 | 1.00 | 60.81 | B |
| ATOM | 113 | CG | LEU | 30 | 60.555 | 5.909 | 52.218 | 1.00 | 61.25 | B |
| ATOM | 114 | CD1 | LEU | 30 | 60.401 | 7.199 | 51.429 | 1.00 | 61.39 | B |
| ATOM | 115 | CD2 | LEU | 30 | 61.935 | 5.810 | 52.856 | 1.00 | 61.13 | B |
| ATOM | 116 | C | LEU | 30 | 60.136 | 4.167 | 55.047 | 1.00 | 62.80 | B |
| ATOM | 117 | O | LEU | 30 | 61.206 | 3.611 | 54.852 | 1.00 | 63.36 | B |
| ATOM | 118 | N | ALA | 31 | 59.736 | 4.545 | 56.257 | 1.00 | 64.56 | B |
| ATOM | 119 | CA | ALA | 31 | 60.565 | 4.326 | 57.440 | 1.00 | 66.24 | B |
| ATOM | 120 | CB | ALA | 31 | 59.999 | 5.104 | 58.617 | 1.00 | 64.93 | B |
| ATOM | 121 | C | ALA | 31 | 60.671 | 2.846 | 57.798 | 1.00 | 68.38 | B |
| ATOM | 122 | O | ALA | 31 | 61.757 | 2.345 | 58.088 | 1.00 | 69.26 | B |
| ATOM | 123 | N | GLU | 32 | 59.537 | 2.153 | 57.781 | 1.00 | 69.84 | B |
| ATOM | 124 | CA | GLU | 32 | 59.492 | 0.734 | 58.107 | 1.00 | 71.88 | B |
| ATOM | 125 | CB | GLU | 32 | 58.038 | 0.275 | 58.225 | 1.00 | 70.67 | B |
| ATOM | 126 | CG | GLU | 32 | 57.338 | 0.752 | 59.487 | 1.00 | 67.99 | B |
| ATOM | 127 | CD | GLU | 32 | 55.831 | 0.607 | 59.412 | 1.00 | 65.98 | B |
| ATOM | 128 | OE1 | GLU | 32 | 55.174 | 0.723 | 60.468 | 1.00 | 65.36 | B |
| ATOM | 129 | OE2 | GLU | 32 | 55.302 | 0.383 | 58.301 | 1.00 | 62.48 | B |
| ATOM | 130 | C | GLU | 32 | 60.232 | −0.143 | 57.097 | 1.00 | 74.40 | B |
| ATOM | 131 | O | GLU | 32 | 61.090 | −0.930 | 57.472 | 1.00 | 74.92 | B |
| ATOM | 132 | N | ARG | 33 | 59.897 | −0.008 | 55.816 | 1.00 | 76.35 | B |
| ATOM | 133 | CA | ARG | 33 | 60.550 | −0.803 | 54.779 | 1.00 | 78.32 | B |
| ATOM | 134 | CB | ARG | 33 | 59.936 | −0.502 | 53.407 | 1.00 | 79.77 | B |
| ATOM | 135 | CG | ARG | 33 | 59.972 | 0.964 | 53.010 | 1.00 | 83.18 | B |
| ATOM | 136 | CD | ARG | 33 | 59.329 | 1.183 | 51.645 | 1.00 | 85.46 | B |
| ATOM | 137 | NE | ARG | 33 | 60.032 | 0.459 | 50.589 | 1.00 | 87.40 | B |
| ATOM | 138 | CZ | ARG | 33 | 61.269 | 0.737 | 50.186 | 1.00 | 88.75 | B |
| ATOM | 139 | NH1 | ARG | 33 | 61.948 | 1.729 | 50.747 | 1.00 | 89.79 | B |
| ATOM | 140 | NH2 | ARG | 33 | 61.828 | 0.019 | 49.221 | 1.00 | 89.07 | B |
| ATOM | 141 | C | ARG | 33 | 62.053 | −0.536 | 54.754 | 1.00 | 78.80 | B |
| ATOM | 142 | O | ARG | 33 | 62.832 | −1.379 | 54.318 | 1.00 | 78.36 | B |
| ATOM | 143 | N | LYS | 34 | 62.448 | 0.644 | 55.226 | 1.00 | 79.39 | B |
| ATOM | 144 | CA | LYS | 34 | 63.853 | 1.029 | 55.284 | 1.00 | 80.19 | B |
| ATOM | 145 | CB | LYS | 34 | 63.984 | 2.543 | 55.504 | 1.00 | 81.11 | B |
| ATOM | 146 | CG | LYS | 34 | 64.392 | 3.347 | 54.267 | 1.00 | 82.59 | B |
| ATOM | 147 | CD | LYS | 34 | 65.910 | 3.501 | 54.147 | 1.00 | 83.41 | B |
| ATOM | 148 | CE | LYS | 34 | 66.604 | 2.186 | 53.810 | 1.00 | 84.19 | B |
| ATOM | 149 | NZ | LYS | 34 | 68.089 | 2.305 | 53.845 | 1.00 | 84.38 | B |
| ATOM | 150 | C | LYS | 34 | 64.539 | 0.285 | 56.423 | 1.00 | 80.45 | B |
| ATOM | 151 | O | LYS | 34 | 65.757 | 0.159 | 56.448 | 1.00 | 81.20 | B |
| ATOM | 152 | N | ALA | 35 | 63.740 | −0.209 | 57.365 | 1.00 | 80.19 | B |
| ATOM | 153 | CA | ALA | 35 | 64.264 | −0.946 | 58.509 | 1.00 | 79.99 | B |
| ATOM | 154 | CB | ALA | 35 | 63.654 | −0.405 | 59.800 | 1.00 | 79.19 | B |
| ATOM | 155 | C | ALA | 35 | 63.966 | −2.441 | 58.372 | 1.00 | 79.54 | B |
| ATOM | 156 | O | ALA | 35 | 64.029 | −3.181 | 59.347 | 1.00 | 79.52 | B |
| ATOM | 157 | N | SER | 36 | 63.650 | −2.870 | 57.150 | 1.00 | 79.23 | B |
| ATOM | 158 | CA | SER | 36 | 63.324 | −4.269 | 56.866 | 1.00 | 78.90 | B |
| ATOM | 159 | CB | SER | 36 | 64.581 | −5.140 | 56.934 | 1.00 | 79.55 | B |
| ATOM | 160 | OG | SER | 36 | 65.497 | −4.786 | 55.913 | 1.00 | 80.94 | B |
| ATOM | 161 | C | SER | 36 | 62.291 | −4.773 | 57.863 | 1.00 | 77.94 | B |
| ATOM | 162 | O | SER | 36 | 62.621 | −5.460 | 58.826 | 1.00 | 78.06 | B |
| ATOM | 163 | N | ALA | 37 | 61.033 | −4.422 | 57.620 | 1.00 | 76.14 | B |
| ATOM | 164 | CA | ALA | 37 | 59.952 | −4.822 | 58.505 | 1.00 | 74.02 | B |
| ATOM | 165 | CB | ALA | 37 | 58.862 | −3.763 | 58.496 | 1.00 | 74.76 | B |
| ATOM | 166 | C | ALA | 37 | 59.370 | −6.177 | 58.128 | 1.00 | 72.27 | B |
| ATOM | 167 | O | ALA | 37 | 59.282 | −6.526 | 56.956 | 1.00 | 71.83 | B |
| ATOM | 168 | N | HIS | 38 | 58.975 | −6.928 | 59.151 | 1.00 | 70.33 | B |
| ATOM | 169 | CA | HIS | 38 | 58.388 | −8.249 | 58.981 | 1.00 | 67.10 | B |
| ATOM | 170 | CB | HIS | 38 | 59.039 | −9.236 | 59.961 | 1.00 | 69.95 | B |
| ATOM | 171 | CG | HIS | 38 | 59.177 | −8.706 | 61.358 | 1.00 | 72.03 | B |
| ATOM | 172 | CD2 | HIS | 38 | 58.589 | −9.085 | 62.518 | 1.00 | 72.68 | B |
| ATOM | 173 | ND1 | HIS | 38 | 60.004 | −7.648 | 61.676 | 1.00 | 72.05 | B |
| ATOM | 174 | CE1 | HIS | 38 | 59.919 | −7.399 | 62.971 | 1.00 | 72.38 | B |
| ATOM | 175 | NE2 | HIS | 38 | 59.067 | −8.256 | 63.505 | 1.00 | 73.14 | B |
| ATOM | 176 | C | HIS | 38 | 56.877 | −8.187 | 59.220 | 1.00 | 63.55 | B |
| ATOM | 177 | O | HIS | 38 | 56.426 | −7.917 | 60.335 | 1.00 | 63.33 | B |
| ATOM | 178 | N | SER | 39 | 56.100 | −8.432 | 58.168 | 1.00 | 58.67 | B |
| ATOM | 179 | CA | SER | 39 | 54.643 | −8.399 | 58.266 | 1.00 | 54.45 | B |
| ATOM | 180 | CB | SER | 39 | 54.005 | −8.478 | 56.879 | 1.00 | 53.84 | B |
| ATOM | 181 | OG | SER | 39 | 52.595 | −8.614 | 56.976 | 1.00 | 49.31 | B |
| ATOM | 182 | C | SER | 39 | 54.081 | −9.519 | 59.122 | 1.00 | 52.25 | B |
| ATOM | 183 | O | SER | 39 | 54.384 | −10.686 | 58.910 | 1.00 | 51.84 | B |
| ATOM | 184 | N | ILE | 40 | 53.251 | −9.149 | 60.089 | 1.00 | 49.22 | B |
| ATOM | 185 | CA | ILE | 40 | 52.631 | −10.122 | 60.967 | 1.00 | 47.52 | B |
| ATOM | 186 | CB | ILE | 40 | 52.679 | −9.674 | 62.444 | 1.00 | 45.91 | B |
| ATOM | 187 | CG2 | ILE | 40 | 54.115 | −9.499 | 62.881 | 1.00 | 44.82 | B |
| ATOM | 188 | CG1 | ILE | 40 | 51.915 | −8.361 | 62.622 | 1.00 | 45.54 | B |
| ATOM | 189 | CD1 | ILE | 40 | 51.580 | −8.050 | 64.066 | 1.00 | 46.62 | B |
| ATOM | 190 | C | ILE | 40 | 51.176 | −10.316 | 60.557 | 1.00 | 47.28 | B |
| ATOM | 191 | O | ILE | 40 | 50.421 | −10.994 | 61.234 | 1.00 | 46.90 | B |
| ATOM | 192 | N | VAL | 41 | 50.798 | −9.718 | 59.433 | 1.00 | 47.41 | B |
| ATOM | 193 | CA | VAL | 41 | 49.430 | −9.824 | 58.939 | 1.00 | 48.95 | B |
| ATOM | 194 | CB | VAL | 41 | 48.713 | −8.450 | 58.983 | 1.00 | 49.16 | B |
| ATOM | 195 | CG1 | VAL | 41 | 47.290 | −8.585 | 58.467 | 1.00 | 49.01 | B |
| ATOM | 196 | CG2 | VAL | 41 | 48.713 | −7.903 | 60.402 | 1.00 | 49.06 | B |
| ATOM | 197 | C | VAL | 41 | 49.395 | −10.347 | 57.509 | 1.00 | 49.67 | B |
| ATOM | 198 | O | VAL | 41 | 50.004 | −9.777 | 56.620 | 1.00 | 49.95 | B |
| ATOM | 199 | N | GLU | 42 | 48.685 | −11.449 | 57.301 | 1.00 | 50.48 | B |
| ATOM | 200 | CA | GLU | 42 | 48.575 | −12.024 | 55.969 | 1.00 | 51.59 | B |
| ATOM | 201 | CB | GLU | 42 | 49.176 | −13.434 | 55.935 | 1.00 | 52.66 | B |
| ATOM | 202 | CG | GLU | 42 | 50.609 | −13.510 | 56.447 | 1.00 | 56.16 | B |
| ATOM | 203 | CD | GLU | 42 | 51.164 | −14.931 | 56.476 | 1.00 | 58.24 | B |
| ATOM | 204 | OE1 | GLU | 42 | 50.430 | −15.854 | 56.899 | 1.00 | 57.80 | B |
| ATOM | 205 | OE2 | GLU | 42 | 52.338 | −15.119 | 56.081 | 1.00 | 58.28 | B |
| ATOM | 206 | C | GLU | 42 | 47.102 | −12.072 | 55.599 | 1.00 | 50.83 | B |
| ATOM | 207 | O | GLU | 42 | 46.283 | −12.604 | 56.343 | 1.00 | 51.55 | B |
| ATOM | 208 | N | CYS | 43 | 46.768 | −11.493 | 54.453 | 1.00 | 49.80 | B |
| ATOM | 209 | CA | CYS | 43 | 45.389 | −11.473 | 53.995 | 1.00 | 49.65 | B |
| ATOM | 210 | CB | CYS | 43 | 45.037 | −10.087 | 53.433 | 1.00 | 49.93 | B |
| ATOM | 211 | SG | CYS | 43 | 45.019 | −8.745 | 54.661 | 1.00 | 48.78 | B |
| ATOM | 212 | C | CYS | 43 | 45.140 | −12.535 | 52.931 | 1.00 | 48.94 | B |
| ATOM | 213 | O | CYS | 43 | 46.010 | −12.833 | 52.123 | 1.00 | 48.97 | B |
| ATOM | 214 | N | ASP | 44 | 43.939 | −13.105 | 52.954 | 1.00 | 49.14 | B |
| ATOM | 215 | CA | ASP | 44 | 43.534 | −14.121 | 51.992 | 1.00 | 48.86 | B |
| ATOM | 216 | CB | ASP | 44 | 43.463 | −15.494 | 52.660 | 1.00 | 50.97 | B |
| ATOM | 217 | CG | ASP | 44 | 43.589 | −16.635 | 51.666 | 1.00 | 52.32 | B |
| ATOM | 218 | OD1 | ASP | 44 | 43.126 | −16.483 | 50.510 | 1.00 | 52.22 | B |
| ATOM | 219 | OD2 | ASP | 44 | 44.147 | −17.689 | 52.048 | 1.00 | 52.81 | B |
| ATOM | 220 | C | ASP | 44 | 42.150 | −13.749 | 51.456 | 1.00 | 48.60 | B |
| ATOM | 221 | O | ASP | 44 | 41.127 | −14.147 | 52.012 | 1.00 | 46.42 | B |
| ATOM | 222 | N | PRO | 45 | 42.108 | −12.969 | 50.364 | 1.00 | 48.35 | B |
| ATOM | 223 | CD | PRO | 45 | 43.252 | −12.517 | 49.557 | 1.00 | 48.19 | B |
| ATOM | 224 | CA | PRO | 45 | 40.847 | −12.540 | 49.755 | 1.00 | 48.75 | B |
| ATOM | 225 | CB | PRO | 45 | 41.307 | −11.680 | 48.584 | 1.00 | 49.00 | B |
| ATOM | 226 | CG | PRO | 45 | 42.617 | −12.306 | 48.211 | 1.00 | 49.04 | B |
| ATOM | 227 | C | PRO | 45 | 39.957 | −13.688 | 49.312 | 1.00 | 50.08 | B |
| ATOM | 228 | O | PRO | 45 | 38.750 | −13.661 | 49.535 | 1.00 | 50.55 | B |
| ATOM | 229 | N | VAL | 46 | 40.561 | −14.693 | 48.683 | 1.00 | 50.66 | B |
| ATOM | 230 | CA | VAL | 46 | 39.818 | −15.851 | 48.213 | 1.00 | 50.49 | B |
| ATOM | 231 | CB | VAL | 46 | 40.745 | −16.853 | 47.500 | 1.00 | 50.30 | B |
| ATOM | 232 | CG1 | VAL | 46 | 39.957 | −18.079 | 47.077 | 1.00 | 49.67 | B |
| ATOM | 233 | CG2 | VAL | 46 | 41.393 | −16.192 | 46.293 | 1.00 | 49.30 | B |
| ATOM | 234 | C | VAL | 46 | 39.145 | −16.545 | 49.389 | 1.00 | 50.88 | B |
| ATOM | 235 | O | VAL | 46 | 37.965 | −16.870 | 49.338 | 1.00 | 52.16 | B |
| ATOM | 236 | N | ARG | 47 | 39.906 | −16.761 | 50.454 | 1.00 | 49.91 | B |
| ATOM | 237 | CA | ARG | 47 | 39.369 | −17.417 | 51.635 | 1.00 | 49.25 | B |
| ATOM | 238 | CB | ARG | 47 | 40.499 | −18.074 | 52.431 | 1.00 | 53.01 | B |
| ATOM | 239 | CG | ARG | 47 | 40.025 | −19.009 | 53.535 | 1.00 | 58.79 | B |
| ATOM | 240 | CD | ARG | 47 | 39.711 | −20.404 | 52.993 | 1.00 | 62.76 | B |
| ATOM | 241 | NE | ARG | 47 | 40.925 | −21.094 | 52.566 | 1.00 | 65.61 | B |
| ATOM | 242 | CZ | ARG | 47 | 41.887 | −21.489 | 53.395 | 1.00 | 67.31 | B |
| ATOM | 243 | NH1 | ARG | 47 | 41.770 | −21.265 | 54.699 | 1.00 | 67.77 | B |
| ATOM | 244 | NH2 | ARG | 47 | 42.970 | −22.093 | 52.922 | 1.00 | 67.97 | B |
| ATOM | 245 | C | ARG | 47 | 38.649 | −16.396 | 52.518 | 1.00 | 46.27 | B |
| ATOM | 246 | O | ARG | 47 | 37.980 | −16.767 | 53.479 | 1.00 | 45.17 | B |
| ATOM | 247 | N | LYS | 48 | 38.789 | −15.116 | 52.167 | 1.00 | 43.30 | B |
| ATOM | 248 | CA | LYS | 48 | 38.191 | −14.003 | 52.911 | 1.00 | 40.30 | B |
| ATOM | 249 | CB | LYS | 48 | 36.660 | −14.063 | 52.861 | 1.00 | 40.48 | B |
| ATOM | 250 | CG | LYS | 48 | 36.074 | −13.999 | 51.466 | 1.00 | 42.10 | B |
| ATOM | 251 | CD | LYS | 48 | 34.566 | −14.224 | 51.491 | 1.00 | 46.49 | B |
| ATOM | 252 | CE | LYS | 48 | 34.011 | −14.463 | 50.088 | 1.00 | 48.94 | B |
| ATOM | 253 | NZ | LYS | 48 | 34.342 | −13.358 | 49.137 | 1.00 | 51.33 | B |
| ATOM | 254 | C | LYS | 48 | 38.649 | −14.040 | 54.364 | 1.00 | 38.40 | B |
| ATOM | 255 | O | LYS | 48 | 37.879 | −13.780 | 55.271 | 1.00 | 37.06 | B |
| ATOM | 256 | N | GLU | 49 | 39.918 | −14.374 | 54.573 | 1.00 | 38.43 | B |
| ATOM | 257 | CA | GLU | 49 | 40.472 | −14.451 | 55.918 | 1.00 | 38.68 | B |
| ATOM | 258 | CB | GLU | 49 | 40.965 | −15.867 | 56.237 | 1.00 | 42.04 | B |
| ATOM | 259 | CG | GLU | 49 | 39.896 | −16.940 | 56.342 | 1.00 | 47.74 | B |
| ATOM | 260 | CD | GLU | 49 | 40.478 | −18.320 | 56.671 | 1.00 | 49.86 | B |
| ATOM | 261 | OE1 | GLU | 49 | 39.706 | −19.305 | 56.666 | 1.00 | 50.42 | B |
| ATOM | 262 | OE2 | GLU | 49 | 41.701 | −18.419 | 56.930 | 1.00 | 49.85 | B |
| ATOM | 263 | C | GLU | 49 | 41.643 | −13.506 | 56.111 | 1.00 | 37.41 | B |
| ATOM | 264 | O | GLU | 49 | 42.273 | −13.066 | 55.158 | 1.00 | 34.84 | B |
| ATOM | 265 | N | VAL | 50 | 41.925 | −13.220 | 57.374 | 1.00 | 36.48 | B |
| ATOM | 266 | CA | VAL | 50 | 43.035 | −12.366 | 57.751 | 1.00 | 37.37 | B |
| ATOM | 267 | CB | VAL | 50 | 42.539 | −10.930 | 58.146 | 1.00 | 37.30 | B |
| ATOM | 268 | CG1 | VAL | 50 | 41.332 | −11.008 | 59.061 | 1.00 | 38.02 | B |
| ATOM | 269 | CG2 | VAL | 50 | 43.655 | −10.153 | 58.813 | 1.00 | 36.20 | B |
| ATOM | 270 | C | VAL | 50 | 43.709 | −13.074 | 58.921 | 1.00 | 36.84 | B |
| ATOM | 271 | O | VAL | 50 | 43.078 | −13.354 | 59.926 | 1.00 | 37.07 | B |
| ATOM | 272 | N | SER | 51 | 44.988 | −13.399 | 58.772 | 1.00 | 37.03 | B |
| ATOM | 273 | CA | SER | 51 | 45.702 | −14.095 | 59.835 | 1.00 | 37.03 | B |
| ATOM | 274 | CB | SER | 51 | 46.315 | −15.390 | 59.294 | 1.00 | 37.38 | B |
| ATOM | 275 | OG | SER | 51 | 46.507 | −16.327 | 60.339 | 1.00 | 38.42 | B |
| ATOM | 276 | C | SER | 51 | 46.791 | −13.217 | 60.436 | 1.00 | 37.30 | B |
| ATOM | 277 | O | SER | 51 | 47.538 | −12.567 | 59.712 | 1.00 | 37.32 | B |
| ATOM | 278 | N | VAL | 52 | 46.870 | −13.207 | 61.764 | 1.00 | 37.43 | B |
| ATOM | 279 | CA | VAL | 52 | 47.861 | −12.398 | 62.476 | 1.00 | 40.09 | B |
| ATOM | 280 | CB | VAL | 52 | 47.170 | −11.380 | 63.433 | 1.00 | 38.82 | B |
| ATOM | 281 | CG1 | VAL | 52 | 48.210 | −10.529 | 64.140 | 1.00 | 38.44 | B |
| ATOM | 282 | CG2 | VAL | 52 | 46.207 | −10.507 | 62.664 | 1.00 | 39.75 | B |
| ATOM | 283 | C | VAL | 52 | 48.814 | −13.254 | 63.307 | 1.00 | 41.41 | B |
| ATOM | 284 | O | VAL | 52 | 48.383 | −14.120 | 64.059 | 1.00 | 42.26 | B |
| ATOM | 285 | N | ARG | 53 | 50.112 | −13.001 | 63.170 | 1.00 | 42.93 | B |
| ATOM | 286 | CA | ARG | 53 | 51.115 | −13.746 | 63.922 | 1.00 | 44.63 | B |
| ATOM | 287 | CB | ARG | 53 | 52.435 | −13.782 | 63.156 | 1.00 | 44.21 | B |
| ATOM | 288 | CG | ARG | 53 | 53.621 | −14.258 | 63.976 | 1.00 | 45.18 | B |
| ATOM | 289 | CD | ARG | 53 | 54.721 | −14.772 | 63.069 | 1.00 | 47.32 | B |
| ATOM | 290 | NE | ARG | 53 | 55.045 | −13.815 | 62.016 | 1.00 | 48.93 | B |
| ATOM | 291 | CZ | ARG | 53 | 55.538 | −14.154 | 60.831 | 1.00 | 48.81 | B |
| ATOM | 292 | NH1 | ARG | 53 | 55.762 | −15.430 | 60.548 | 1.00 | 49.29 | B |
| ATOM | 293 | NH2 | ARG | 53 | 55.804 | −13.221 | 59.928 | 1.00 | 50.89 | B |
| ATOM | 294 | C | ARG | 53 | 51.333 | −13.130 | 65.298 | 1.00 | 46.43 | B |
| ATOM | 295 | O | ARG | 53 | 51.867 | −12.030 | 65.420 | 1.00 | 47.02 | B |
| ATOM | 296 | N | THR | 54 | 50.915 | −13.855 | 66.331 | 1.00 | 48.25 | B |
| ATOM | 297 | CA | THR | 54 | 51.052 | −13.401 | 67.711 | 1.00 | 50.92 | B |
| ATOM | 298 | CB | THR | 54 | 49.768 | −13.683 | 68.512 | 1.00 | 50.31 | B |
| ATOM | 299 | OG1 | THR | 54 | 49.572 | −15.098 | 68.631 | 1.00 | 50.23 | B |
| ATOM | 300 | CG2 | THR | 54 | 48.567 | −13.078 | 67.810 | 1.00 | 50.24 | B |
| ATOM | 301 | C | THR | 54 | 52.211 | −14.097 | 68.412 | 1.00 | 53.34 | B |
| ATOM | 302 | O | THR | 54 | 52.551 | −13.769 | 69.538 | 1.00 | 53.13 | B |
| ATOM | 303 | N | GLY | 55 | 52.815 | −15.059 | 67.726 | 1.00 | 57.17 | B |
| ATOM | 304 | CA | GLY | 55 | 53.917 | −15.805 | 68.303 | 1.00 | 61.42 | B |
| ATOM | 305 | C | GLY | 55 | 55.300 | −15.366 | 67.868 | 1.00 | 64.33 | B |
| ATOM | 306 | O | GLY | 55 | 55.566 | −14.175 | 67.715 | 1.00 | 65.05 | B |
| ATOM | 307 | N | GLY | 56 | 56.181 | −16.346 | 67.672 | 1.00 | 66.22 | B |
| ATOM | 308 | CA | GLY | 56 | 57.548 | −16.061 | 67.272 | 1.00 | 68.09 | B |
| ATOM | 309 | C | GLY | 56 | 57.760 | −15.914 | 65.777 | 1.00 | 69.96 | B |
| ATOM | 310 | O | GLY | 56 | 56.950 | −15.305 | 65.084 | 1.00 | 70.41 | B |
| ATOM | 311 | N | LEU | 57 | 58.860 | −16.484 | 65.288 | 1.00 | 71.01 | B |
| ATOM | 312 | CA | LEU | 57 | 59.220 | −16.421 | 63.873 | 1.00 | 70.64 | B |
| ATOM | 313 | CB | LEU | 57 | 60.702 | −16.771 | 63.704 | 1.00 | 71.42 | B |
| ATOM | 314 | CG | LEU | 57 | 61.326 | −17.671 | 64.778 | 1.00 | 71.92 | B |
| ATOM | 315 | CD1 | LEU | 57 | 60.653 | −19.034 | 64.777 | 1.00 | 72.30 | B |
| ATOM | 316 | CD2 | LEU | 57 | 62.819 | −17.813 | 64.522 | 1.00 | 72.27 | B |
| ATOM | 317 | C | LEU | 57 | 58.366 | −17.311 | 62.973 | 1.00 | 70.34 | B |
| ATOM | 318 | O | LEU | 57 | 57.535 | −18.083 | 63.450 | 1.00 | 69.85 | B |
| ATOM | 319 | N | ALA | 58 | 58.589 | −17.189 | 61.667 | 1.00 | 69.38 | B |
| ATOM | 320 | CA | ALA | 58 | 57.852 | −17.959 | 60.669 | 1.00 | 68.14 | B |
| ATOM | 321 | CB | ALA | 58 | 58.169 | −17.430 | 59.268 | 1.00 | 68.25 | B |
| ATOM | 322 | C | ALA | 58 | 58.129 | −19.462 | 60.742 | 1.00 | 66.52 | B |
| ATOM | 323 | O | ALA | 58 | 57.262 | −20.268 | 60.433 | 1.00 | 66.64 | B |
| ATOM | 324 | N | ASP | 59 | 59.343 | −19.825 | 61.150 | 1.00 | 64.49 | B |
| ATOM | 325 | CA | ASP | 59 | 59.743 | −21.226 | 61.270 | 1.00 | 62.67 | B |
| ATOM | 326 | CB | ASP | 59 | 61.183 | −21.310 | 61.798 | 1.00 | 62.19 | B |
| ATOM | 327 | CG | ASP | 59 | 61.589 | −22.724 | 62.197 | 1.00 | 61.33 | B |
| ATOM | 328 | OD1 | ASP | 59 | 61.727 | −23.594 | 61.307 | 1.00 | 59.84 | B |
| ATOM | 329 | OD2 | ASP | 59 | 61.772 | −22.963 | 63.410 | 1.00 | 60.73 | B |
| ATOM | 330 | C | ASP | 59 | 58.801 | −21.994 | 62.201 | 1.00 | 61.33 | B |
| ATOM | 331 | O | ASP | 59 | 58.542 | −23.182 | 62.005 | 1.00 | 60.81 | B |
| ATOM | 332 | N | LYS | 60 | 58.287 | −21.302 | 63.211 | 1.00 | 59.03 | B |
| ATOM | 333 | CA | LYS | 60 | 57.376 | −21.897 | 64.179 | 1.00 | 57.28 | B |
| ATOM | 334 | CB | LYS | 60 | 58.147 | −22.816 | 65.134 | 1.00 | 57.38 | B |
| ATOM | 335 | CG | LYS | 60 | 57.281 | −23.524 | 66.164 | 1.00 | 57.92 | B |
| ATOM | 336 | CD | LYS | 60 | 58.117 | −24.299 | 67.172 | 1.00 | 58.61 | B |
| ATOM | 337 | CE | LYS | 60 | 57.247 | −24.930 | 68.245 | 1.00 | 58.86 | B |
| ATOM | 338 | NZ | LYS | 60 | 58.064 | −25.535 | 69.333 | 1.00 | 59.92 | B |
| ATOM | 339 | C | LYS | 60 | 56.710 | −20.771 | 64.968 | 1.00 | 55.75 | B |
| ATOM | 340 | O | LYS | 60 | 57.391 | −19.942 | 65.574 | 1.00 | 55.85 | B |
| ATOM | 341 | N | SER | 61 | 55.381 | −20.735 | 64.953 | 1.00 | 52.88 | B |
| ATOM | 342 | CA | SER | 61 | 54.655 | −19.692 | 65.666 | 1.00 | 50.87 | B |
| ATOM | 343 | CB | SER | 61 | 54.863 | −18.343 | 64.967 | 1.00 | 50.80 | B |
| ATOM | 344 | OG | SER | 61 | 54.294 | −18.346 | 63.667 | 1.00 | 48.16 | B |
| ATOM | 345 | C | SER | 61 | 53.158 | −19.957 | 65.796 | 1.00 | 50.20 | B |
| ATOM | 346 | O | SER | 61 | 52.630 | −20.909 | 65.245 | 1.00 | 49.59 | B |
| ATOM | 347 | N | SER | 62 | 52.493 | −19.086 | 66.547 | 1.00 | 49.11 | B |
| ATOM | 348 | CA | SER | 62 | 51.055 | −19.170 | 66.752 | 1.00 | 48.21 | B |
| ATOM | 349 | CB | SER | 62 | 50.732 | −19.101 | 68.248 | 1.00 | 48.08 | B |
| ATOM | 350 | OG | SER | 62 | 51.371 | −17.993 | 68.858 | 1.00 | 48.30 | B |
| ATOM | 351 | C | SER | 62 | 50.421 | −17.990 | 66.010 | 1.00 | 48.13 | B |
| ATOM | 352 | O | SER | 62 | 51.097 | −17.016 | 65.703 | 1.00 | 47.13 | B |
| ATOM | 353 | N | ARG | 63 | 49.129 | −18.085 | 65.712 | 1.00 | 47.13 | B |
| ATOM | 354 | CA | ARG | 63 | 48.441 | −17.015 | 64.998 | 1.00 | 45.05 | B |
| ATOM | 355 | CB | ARG | 63 | 48.539 | −17.231 | 63.481 | 1.00 | 44.51 | B |
| ATOM | 356 | CG | ARG | 63 | 49.960 | −17.194 | 62.925 | 1.00 | 44.98 | B |
| ATOM | 357 | CD | ARG | 63 | 49.976 | −17.466 | 61.428 | 1.00 | 46.63 | B |
| ATOM | 358 | NE | ARG | 63 | 49.443 | −16.349 | 60.645 | 1.00 | 48.69 | B |
| ATOM | 359 | CZ | ARG | 63 | 50.148 | −15.285 | 60.263 | 1.00 | 48.66 | B |
| ATOM | 360 | NH1 | ARG | 63 | 51.429 | −15.178 | 60.587 | 1.00 | 49.48 | B |
| ATOM | 361 | NH2 | ARG | 63 | 49.574 | −14.329 | 59.545 | 1.00 | 48.53 | B |
| ATOM | 362 | C | ARG | 63 | 46.975 | −16.918 | 65.401 | 1.00 | 43.84 | B |
| ATOM | 363 | O | ARG | 63 | 46.477 | −17.726 | 66.176 | 1.00 | 44.06 | B |
| ATOM | 364 | N | LYS | 64 | 46.305 | −15.902 | 64.868 | 1.00 | 42.24 | B |
| ATOM | 365 | CA | LYS | 64 | 44.892 | −15.652 | 65.124 | 1.00 | 40.40 | B |
| ATOM | 366 | CB | LYS | 64 | 44.723 | −14.434 | 66.032 | 1.00 | 41.92 | B |
| ATOM | 367 | CG | LYS | 64 | 45.181 | −14.635 | 67.470 | 1.00 | 43.37 | B |
| ATOM | 368 | CD | LYS | 64 | 44.088 | −15.261 | 68.317 | 1.00 | 43.81 | B |
| ATOM | 369 | CE | LYS | 64 | 44.446 | −15.213 | 69.794 | 1.00 | 45.77 | B |
| ATOM | 370 | NZ | LYS | 64 | 43.374 | −15.792 | 70.658 | 1.00 | 46.88 | B |
| ATOM | 371 | C | LYS | 64 | 44.257 | −15.369 | 63.771 | 1.00 | 39.22 | B |
| ATOM | 372 | O | LYS | 64 | 44.631 | −14.405 | 63.102 | 1.00 | 39.99 | B |
| ATOM | 373 | N | THR | 65 | 43.312 | −16.210 | 63.361 | 1.00 | 36.46 | B |
| ATOM | 374 | CA | THR | 65 | 42.656 | −16.031 | 62.074 | 1.00 | 34.76 | B |
| ATOM | 375 | CB | THR | 65 | 42.745 | −17.323 | 61.212 | 1.00 | 35.41 | B |
| ATOM | 376 | OG1 | THR | 65 | 44.118 | −17.692 | 61.041 | 1.00 | 32.86 | B |
| ATOM | 377 | CG2 | THR | 65 | 42.130 | −17.090 | 59.826 | 1.00 | 36.73 | B |
| ATOM | 378 | C | THR | 65 | 41.194 | −15.638 | 62.238 | 1.00 | 34.16 | B |
| ATOM | 379 | O | THR | 65 | 40.477 | −16.200 | 63.070 | 1.00 | 35.43 | B |
| ATOM | 380 | N | TYR | 66 | 40.764 | −14.660 | 61.448 | 1.00 | 30.66 | B |
| ATOM | 381 | CA | TYR | 66 | 39.391 | −14.181 | 61.488 | 1.00 | 28.38 | B |
| ATOM | 382 | CB | TYR | 66 | 39.337 | −12.765 | 62.072 | 1.00 | 25.32 | B |
| ATOM | 383 | CG | TYR | 66 | 39.886 | −12.652 | 63.473 | 1.00 | 22.38 | B |
| ATOM | 384 | CD1 | TYR | 66 | 41.255 | −12.566 | 63.710 | 1.00 | 20.36 | B |
| ATOM | 385 | CE1 | TYR | 66 | 41.753 | −12.475 | 65.011 | 1.00 | 19.50 | B |
| ATOM | 386 | CD2 | TYR | 66 | 39.027 | −12.647 | 64.569 | 1.00 | 22.45 | B |
| ATOM | 387 | CE2 | TYR | 66 | 39.506 | −12.559 | 65.868 | 1.00 | 19.18 | B |
| ATOM | 388 | CZ | TYR | 66 | 40.865 | −12.470 | 66.086 | 1.00 | 21.06 | B |
| ATOM | 389 | OH | TYR | 66 | 41.317 | −12.358 | 67.391 | 1.00 | 25.17 | B |
| ATOM | 390 | C | TYR | 66 | 38.815 | −14.171 | 60.076 | 1.00 | 29.18 | B |
| ATOM | 391 | O | TYR | 66 | 39.537 | −13.953 | 59.108 | 1.00 | 29.59 | B |
| ATOM | 392 | N | THR | 67 | 37.514 | −14.418 | 59.963 | 1.00 | 30.96 | B |
| ATOM | 393 | CA | THR | 67 | 36.854 | −14.420 | 58.662 | 1.00 | 31.82 | B |
| ATOM | 394 | CB | THR | 67 | 36.083 | −15.742 | 58.418 | 1.00 | 31.49 | B |
| ATOM | 395 | OG1 | THR | 67 | 36.983 | −16.849 | 58.543 | 1.00 | 35.18 | B |
| ATOM | 396 | CG2 | THR | 67 | 35.482 | −15.759 | 57.016 | 1.00 | 30.30 | B |
| ATOM | 397 | C | THR | 67 | 35.873 | −13.252 | 58.565 | 1.00 | 31.85 | B |
| ATOM | 398 | O | THR | 67 | 35.100 | −12.996 | 59.504 | 1.00 | 32.04 | B |
| ATOM | 399 | N | PHE | 68 | 35.923 | −12.536 | 57.442 | 1.00 | 29.70 | B |
| ATOM | 400 | CA | PHE | 68 | 35.029 | −11.400 | 57.203 | 1.00 | 31.18 | B |
| ATOM | 401 | CB | PHE | 68 | 35.785 | −10.063 | 57.305 | 1.00 | 29.26 | B |
| ATOM | 402 | CG | PHE | 68 | 36.374 | −9.797 | 58.658 | 1.00 | 27.25 | B |
| ATOM | 403 | CD1 | PHE | 68 | 37.617 | −10.309 | 59.001 | 1.00 | 28.36 | B |
| ATOM | 404 | CD2 | PHE | 68 | 35.666 | −9.071 | 59.611 | 1.00 | 28.98 | B |
| ATOM | 405 | CE1 | PHE | 68 | 38.147 | −10.110 | 60.277 | 1.00 | 27.66 | B |
| ATOM | 406 | CE2 | PHE | 68 | 36.188 | −8.867 | 60.894 | 1.00 | 27.30 | B |
| ATOM | 407 | CZ | PHE | 68 | 37.430 | −9.388 | 61.225 | 1.00 | 26.68 | B |
| ATOM | 408 | C | PHE | 68 | 34.418 | −11.527 | 55.815 | 1.00 | 30.88 | B |
| ATOM | 409 | O | PHE | 68 | 34.814 | −12.385 | 55.032 | 1.00 | 32.33 | B |
| ATOM | 410 | N | ASP | 69 | 33.452 | −10.670 | 55.514 | 1.00 | 30.45 | B |
| ATOM | 411 | CA | ASP | 69 | 32.796 | −10.702 | 54.212 | 1.00 | 31.77 | B |
| ATOM | 412 | CB | ASP | 69 | 31.636 | −9.698 | 54.185 | 1.00 | 33.60 | B |
| ATOM | 413 | CG | ASP | 69 | 30.590 | −9.988 | 55.258 | 1.00 | 36.34 | B |
| ATOM | 414 | OD1 | ASP | 69 | 30.514 | −9.221 | 56.254 | 1.00 | 35.89 | B |
| ATOM | 415 | OD2 | ASP | 69 | 29.856 | −10.995 | 55.112 | 1.00 | 33.96 | B |
| ATOM | 416 | C | ASP | 69 | 33.775 | −10.414 | 53.078 | 1.00 | 30.67 | B |
| ATOM | 417 | O | ASP | 69 | 33.594 | −10.882 | 51.970 | 1.00 | 31.26 | B |
| ATOM | 418 | N | MET | 70 | 34.816 | −9.646 | 53.377 | 1.00 | 31.20 | B |
| ATOM | 419 | CA | MET | 70 | 35.836 | −9.294 | 52.394 | 1.00 | 31.00 | B |
| ATOM | 420 | CB | MET | 70 | 35.396 | −8.081 | 51.567 | 1.00 | 33.24 | B |
| ATOM | 421 | CG | MET | 70 | 34.253 | −8.330 | 50.598 | 1.00 | 35.15 | B |
| ATOM | 422 | SD | MET | 70 | 33.994 | −6.921 | 49.476 | 1.00 | 43.03 | B |
| ATOM | 423 | CE | MET | 70 | 32.288 | −6.531 | 49.777 | 1.00 | 42.27 | B |
| ATOM | 424 | C | MET | 70 | 37.158 | −8.978 | 53.090 | 1.00 | 29.72 | B |
| ATOM | 425 | O | MET | 70 | 37.186 | −8.682 | 54.271 | 1.00 | 29.23 | B |
| ATOM | 426 | N | VAL | 71 | 38.257 | −9.052 | 52.353 | 1.00 | 28.80 | B |
| ATOM | 427 | CA | VAL | 71 | 39.561 | −8.765 | 52.929 | 1.00 | 30.15 | B |
| ATOM | 428 | CB | VAL | 71 | 40.256 | −10.054 | 53.443 | 1.00 | 31.84 | B |
| ATOM | 429 | CG1 | VAL | 71 | 41.603 | −9.713 | 54.060 | 1.00 | 33.61 | B |
| ATOM | 430 | CG2 | VAL | 71 | 39.388 | −10.738 | 54.471 | 1.00 | 31.83 | B |
| ATOM | 431 | C | VAL | 71 | 40.439 | −8.102 | 51.878 | 1.00 | 29.25 | B |
| ATOM | 432 | O | VAL | 71 | 40.471 | −8.526 | 50.734 | 1.00 | 30.25 | B |
| ATOM | 433 | N | PHE | 72 | 41.146 | −7.053 | 52.285 | 1.00 | 30.15 | B |
| ATOM | 434 | CA | PHE | 72 | 42.015 | −6.306 | 51.384 | 1.00 | 30.67 | B |
| ATOM | 435 | CB | PHE | 72 | 41.445 | −4.905 | 51.152 | 1.00 | 28.16 | B |
| ATOM | 436 | CG | PHE | 72 | 40.060 | −4.903 | 50.573 | 1.00 | 27.42 | B |
| ATOM | 437 | CD1 | PHE | 72 | 39.854 | −5.145 | 49.220 | 1.00 | 26.23 | B |
| ATOM | 438 | CD2 | PHE | 72 | 38.955 | −4.686 | 51.390 | 1.00 | 26.64 | B |
| ATOM | 439 | CE1 | PHE | 72 | 38.565 | −5.171 | 48.688 | 1.00 | 25.66 | B |
| ATOM | 440 | CE2 | PHE | 72 | 37.664 | −4.709 | 50.868 | 1.00 | 25.86 | B |
| ATOM | 441 | CZ | PHE | 72 | 37.469 | −4.954 | 49.516 | 1.00 | 24.73 | B |
| ATOM | 442 | C | PHE | 72 | 43.428 | −6.188 | 51.940 | 1.00 | 31.84 | B |
| ATOM | 443 | O | PHE | 72 | 43.646 | −5.560 | 52.973 | 1.00 | 30.82 | B |
| ATOM | 444 | N | GLY | 73 | 44.385 | −6.797 | 51.247 | 1.00 | 32.27 | B |
| ATOM | 445 | CA | GLY | 73 | 45.757 | −6.727 | 51.697 | 1.00 | 32.67 | B |
| ATOM | 446 | C | GLY | 73 | 46.358 | −5.377 | 51.366 | 1.00 | 33.72 | B |
| ATOM | 447 | O | GLY | 73 | 45.730 | −4.553 | 50.707 | 1.00 | 33.21 | B |
| ATOM | 448 | N | ALA | 74 | 47.589 | −5.163 | 51.815 | 1.00 | 34.20 | B |
| ATOM | 449 | CA | ALA | 74 | 48.296 | −3.911 | 51.583 | 1.00 | 35.80 | B |
| ATOM | 450 | CB | ALA | 74 | 49.615 | −3.929 | 52.329 | 1.00 | 35.10 | B |
| ATOM | 451 | C | ALA | 74 | 48.547 | −3.664 | 50.100 | 1.00 | 37.02 | B |
| ATOM | 452 | O | ALA | 74 | 49.235 | −2.734 | 49.730 | 1.00 | 38.45 | B |
| ATOM | 453 | N | SER | 75 | 47.971 | −4.498 | 49.250 | 1.00 | 38.40 | B |
| ATOM | 454 | CA | SER | 75 | 48.179 | −4.356 | 47.821 | 1.00 | 40.23 | B |
| ATOM | 455 | CB | SER | 75 | 48.437 | −5.733 | 47.204 | 1.00 | 40.06 | B |
| ATOM | 456 | OG | SER | 75 | 47.371 | −6.617 | 47.504 | 1.00 | 38.50 | B |
| ATOM | 457 | C | SER | 75 | 46.990 | −3.701 | 47.126 | 1.00 | 40.71 | B |
| ATOM | 458 | O | SER | 75 | 47.155 | −3.026 | 46.109 | 1.00 | 40.44 | B |
| ATOM | 459 | N | THR | 76 | 45.795 | −3.917 | 47.677 | 1.00 | 40.56 | B |
| ATOM | 460 | CA | THR | 76 | 44.568 | −3.365 | 47.107 | 1.00 | 40.11 | B |
| ATOM | 461 | CB | THR | 76 | 43.325 | −3.769 | 47.960 | 1.00 | 41.15 | B |
| ATOM | 462 | OG1 | THR | 76 | 43.690 | −3.865 | 49.342 | 1.00 | 43.22 | B |
| ATOM | 463 | CG2 | THR | 76 | 42.774 | −5.118 | 47.498 | 1.00 | 43.01 | B |
| ATOM | 464 | C | THR | 76 | 44.615 | −1.849 | 46.937 | 1.00 | 38.50 | B |
| ATOM | 465 | O | THR | 76 | 45.071 | −1.119 | 47.819 | 1.00 | 38.53 | B |
| ATOM | 466 | N | LYS | 77 | 44.152 | −1.385 | 45.785 | 1.00 | 36.21 | B |
| ATOM | 467 | CA | LYS | 77 | 44.135 | 0.036 | 45.483 | 1.00 | 34.26 | B |
| ATOM | 468 | CB | LYS | 77 | 44.482 | 0.243 | 44.011 | 1.00 | 36.10 | B |
| ATOM | 469 | CG | LYS | 77 | 45.901 | −0.174 | 43.651 | 1.00 | 39.66 | B |
| ATOM | 470 | CD | LYS | 77 | 46.138 | −0.013 | 42.153 | 1.00 | 43.10 | B |
| ATOM | 471 | CE | LYS | 77 | 47.538 | −0.446 | 41.749 | 1.00 | 44.09 | B |
| ATOM | 472 | NZ | LYS | 77 | 47.693 | −0.451 | 40.261 | 1.00 | 46.93 | B |
| ATOM | 473 | C | LYS | 77 | 42.776 | 0.662 | 45.799 | 1.00 | 32.74 | B |
| ATOM | 474 | O | LYS | 77 | 41.807 | −0.045 | 46.049 | 1.00 | 30.61 | B |
| ATOM | 475 | N | GLN | 78 | 42.729 | 1.994 | 45.800 | 1.00 | 31.08 | B |
| ATOM | 476 | CA | GLN | 78 | 41.499 | 2.731 | 46.084 | 1.00 | 29.81 | B |
| ATOM | 477 | CB | GLN | 78 | 41.718 | 4.241 | 45.896 | 1.00 | 29.96 | B |
| ATOM | 478 | CG | GLN | 78 | 42.791 | 4.867 | 46.790 | 1.00 | 28.93 | B |
| ATOM | 479 | CD | GLN | 78 | 42.339 | 5.029 | 48.224 | 1.00 | 28.69 | B |
| ATOM | 480 | OE1 | GLN | 78 | 41.731 | 4.136 | 48.789 | 1.00 | 28.17 | B |
| ATOM | 481 | NE2 | GLN | 78 | 42.647 | 6.177 | 48.822 | 1.00 | 28.63 | B |
| ATOM | 482 | C | GLN | 78 | 40.371 | 2.273 | 45.160 | 1.00 | 29.13 | B |
| ATOM | 483 | O | GLN | 78 | 39.255 | 2.045 | 45.597 | 1.00 | 28.04 | B |
| ATOM | 484 | N | ILE | 79 | 40.687 | 2.140 | 43.877 | 1.00 | 27.65 | B |
| ATOM | 485 | CA | ILE | 79 | 39.710 | 1.730 | 42.874 | 1.00 | 28.90 | B |
| ATOM | 486 | CB | ILE | 79 | 40.369 | 1.664 | 41.472 | 1.00 | 28.34 | B |
| ATOM | 487 | CG2 | ILE | 79 | 41.411 | 0.564 | 41.442 | 1.00 | 30.45 | B |
| ATOM | 488 | CG1 | ILE | 79 | 39.316 | 1.396 | 40.400 | 1.00 | 29.43 | B |
| ATOM | 489 | CD1 | ILE | 79 | 38.333 | 2.517 | 40.226 | 1.00 | 30.66 | B |
| ATOM | 490 | C | ILE | 79 | 39.055 | 0.377 | 43.191 | 1.00 | 28.47 | B |
| ATOM | 491 | O | ILE | 79 | 37.867 | 0.175 | 42.938 | 1.00 | 27.79 | B |
| ATOM | 492 | N | ASP | 80 | 39.829 | −0.548 | 43.749 | 1.00 | 28.15 | B |
| ATOM | 493 | CA | ASP | 80 | 39.296 | −1.866 | 44.076 | 1.00 | 27.60 | B |
| ATOM | 494 | CB | ASP | 80 | 40.435 | −2.865 | 44.316 | 1.00 | 27.34 | B |
| ATOM | 495 | CG | ASP | 80 | 41.439 | −2.908 | 43.164 | 1.00 | 29.59 | B |
| ATOM | 496 | OD1 | ASP | 80 | 41.018 | −2.784 | 41.987 | 1.00 | 27.17 | B |
| ATOM | 497 | OD2 | ASP | 80 | 42.648 | −3.078 | 43.445 | 1.00 | 29.79 | B |
| ATOM | 498 | C | ASP | 80 | 38.395 | −1.800 | 45.303 | 1.00 | 27.71 | B |
| ATOM | 499 | O | ASP | 80 | 37.394 | −2.492 | 45.383 | 1.00 | 27.27 | B |
| ATOM | 500 | N | VAL | 81 | 38.761 | −0.964 | 46.265 | 1.00 | 28.05 | B |
| ATOM | 501 | CA | VAL | 81 | 37.947 | −0.820 | 47.460 | 1.00 | 27.29 | B |
| ATOM | 502 | CB | VAL | 81 | 38.618 | 0.115 | 48.495 | 1.00 | 25.22 | B |
| ATOM | 503 | CG1 | VAL | 81 | 37.662 | 0.394 | 49.633 | 1.00 | 21.33 | B |
| ATOM | 504 | CG2 | VAL | 81 | 39.890 | −0.532 | 49.036 | 1.00 | 23.97 | B |
| ATOM | 505 | C | VAL | 81 | 36.588 | −0.244 | 47.079 | 1.00 | 28.97 | B |
| ATOM | 506 | O | VAL | 81 | 35.555 | −0.682 | 47.590 | 1.00 | 29.68 | B |
| ATOM | 507 | N | TYR | 82 | 36.593 | 0.721 | 46.162 | 1.00 | 28.62 | B |
| ATOM | 508 | CA | TYR | 82 | 35.364 | 1.368 | 45.723 | 1.00 | 30.02 | B |
| ATOM | 509 | CB | TYR | 82 | 35.693 | 2.640 | 44.924 | 1.00 | 31.49 | B |
| ATOM | 510 | CG | TYR | 82 | 34.472 | 3.389 | 44.443 | 1.00 | 33.00 | B |
| ATOM | 511 | CD1 | TYR | 82 | 33.934 | 3.144 | 43.180 | 1.00 | 34.00 | B |
| ATOM | 512 | CE1 | TYR | 82 | 32.776 | 3.781 | 42.762 | 1.00 | 37.72 | B |
| ATOM | 513 | CD2 | TYR | 82 | 33.817 | 4.299 | 45.278 | 1.00 | 32.60 | B |
| ATOM | 514 | CE2 | TYR | 82 | 32.659 | 4.938 | 44.871 | 1.00 | 36.04 | B |
| ATOM | 515 | CZ | TYR | 82 | 32.142 | 4.676 | 43.613 | 1.00 | 39.42 | B |
| ATOM | 516 | OH | TYR | 82 | 30.992 | 5.316 | 43.203 | 1.00 | 42.75 | B |
| ATOM | 517 | C | TYR | 82 | 34.456 | 0.451 | 44.906 | 1.00 | 30.88 | B |
| ATOM | 518 | O | TYR | 82 | 33.264 | 0.363 | 45.168 | 1.00 | 30.76 | B |
| ATOM | 519 | N | ARG | 83 | 35.021 | −0.223 | 43.910 | 1.00 | 32.85 | B |
| ATOM | 520 | CA | ARG | 83 | 34.239 | −1.136 | 43.077 | 1.00 | 34.09 | B |
| ATOM | 521 | CB | ARG | 83 | 35.120 | −1.702 | 41.965 | 1.00 | 35.60 | B |
| ATOM | 522 | CG | ARG | 83 | 35.333 | −0.749 | 40.798 | 1.00 | 42.48 | B |
| ATOM | 523 | CD | ARG | 83 | 36.652 | −1.013 | 40.072 | 1.00 | 46.99 | B |
| ATOM | 524 | NE | ARG | 83 | 36.734 | −2.358 | 39.503 | 1.00 | 53.06 | B |
| ATOM | 525 | CZ | ARG | 83 | 36.100 | −2.758 | 38.404 | 1.00 | 56.78 | B |
| ATOM | 526 | NH1 | ARG | 83 | 35.323 | −1.914 | 37.735 | 1.00 | 57.61 | B |
| ATOM | 527 | NH2 | ARG | 83 | 36.254 | −4.004 | 37.967 | 1.00 | 57.03 | B |
| ATOM | 528 | C | ARG | 83 | 33.630 | −2.277 | 43.895 | 1.00 | 33.36 | B |
| ATOM | 529 | O | ARG | 83 | 32.492 | −2.674 | 43.667 | 1.00 | 34.00 | B |
| ATOM | 530 | N | SER | 84 | 34.390 | −2.785 | 44.860 | 1.00 | 31.69 | B |
| ATOM | 531 | CA | SER | 84 | 33.956 | −3.899 | 45.701 | 1.00 | 30.91 | B |
| ATOM | 532 | CB | SER | 84 | 35.180 | −4.582 | 46.322 | 1.00 | 31.88 | B |
| ATOM | 533 | OG | SER | 84 | 36.115 | −4.951 | 45.324 | 1.00 | 34.36 | B |
| ATOM | 534 | C | SER | 84 | 32.983 | −3.535 | 46.816 | 1.00 | 30.39 | B |
| ATOM | 535 | O | SER | 84 | 31.963 | −4.195 | 47.007 | 1.00 | 30.60 | B |
| ATOM | 536 | N | VAL | 85 | 33.299 | −2.489 | 47.568 | 1.00 | 29.66 | B |
| ATOM | 537 | CA | VAL | 85 | 32.432 | −2.091 | 48.663 | 1.00 | 28.01 | B |
| ATOM | 538 | CB | VAL | 85 | 33.255 | −1.652 | 49.887 | 1.00 | 27.01 | B |
| ATOM | 539 | CG1 | VAL | 85 | 32.336 | −1.128 | 50.971 | 1.00 | 26.26 | B |
| ATOM | 540 | CG2 | VAL | 85 | 34.080 | −2.815 | 50.407 | 1.00 | 26.27 | B |
| ATOM | 541 | C | VAL | 85 | 31.445 | −0.983 | 48.337 | 1.00 | 27.47 | B |
| ATOM | 542 | O | VAL | 85 | 30.249 | −1.149 | 48.498 | 1.00 | 28.23 | B |
| ATOM | 543 | N | VAL | 86 | 31.960 | 0.145 | 47.868 | 1.00 | 28.02 | B |
| ATOM | 544 | CA | VAL | 86 | 31.132 | 1.313 | 47.585 | 1.00 | 28.51 | B |
| ATOM | 545 | CB | VAL | 86 | 32.004 | 2.568 | 47.370 | 1.00 | 26.65 | B |
| ATOM | 546 | CG1 | VAL | 86 | 31.180 | 3.808 | 47.625 | 1.00 | 25.89 | B |
| ATOM | 547 | CG2 | VAL | 86 | 33.220 | 2.532 | 48.267 | 1.00 | 25.41 | B |
| ATOM | 548 | C | VAL | 86 | 30.150 | 1.224 | 46.425 | 1.00 | 29.30 | B |
| ATOM | 549 | O | VAL | 86 | 28.959 | 1.479 | 46.599 | 1.00 | 28.44 | B |
| ATOM | 550 | N | CYS | 87 | 30.649 | 0.881 | 45.244 | 1.00 | 29.85 | B |
| ATOM | 551 | CA | CYS | 87 | 29.802 | 0.786 | 44.064 | 1.00 | 33.34 | B |
| ATOM | 552 | CB | CYS | 87 | 30.549 | 0.025 | 42.965 | 1.00 | 36.49 | B |
| ATOM | 553 | SG | CYS | 87 | 29.936 | 0.313 | 41.286 | 1.00 | 43.07 | B |
| ATOM | 554 | C | CYS | 87 | 28.445 | 0.131 | 44.373 | 1.00 | 34.93 | B |
| ATOM | 555 | O | CYS | 87 | 27.396 | 0.670 | 44.026 | 1.00 | 34.18 | B |
| ATOM | 556 | N | PRO | 88 | 28.452 | −1.035 | 45.045 | 1.00 | 35.57 | B |
| ATOM | 557 | CD | PRO | 88 | 29.603 | −1.876 | 45.420 | 1.00 | 37.48 | B |
| ATOM | 558 | CA | PRO | 88 | 27.195 | −1.715 | 45.378 | 1.00 | 35.50 | B |
| ATOM | 559 | CB | PRO | 88 | 27.664 | −2.989 | 46.078 | 1.00 | 35.52 | B |
| ATOM | 560 | CG | PRO | 88 | 28.984 | −3.247 | 45.464 | 1.00 | 36.85 | B |
| ATOM | 561 | C | PRO | 88 | 26.295 | −0.874 | 46.287 | 1.00 | 35.13 | B |
| ATOM | 562 | O | PRO | 88 | 25.099 | −0.765 | 46.050 | 1.00 | 35.74 | B |
| ATOM | 563 | N | ILE | 89 | 26.885 | −0.288 | 47.327 | 1.00 | 34.00 | B |
| ATOM | 564 | CA | ILE | 89 | 26.140 | 0.535 | 48.279 | 1.00 | 33.52 | B |
| ATOM | 565 | CB | ILE | 89 | 27.031 | 0.978 | 49.465 | 1.00 | 33.84 | B |
| ATOM | 566 | CG2 | ILE | 89 | 26.250 | 1.910 | 50.384 | 1.00 | 34.73 | B |
| ATOM | 567 | CG1 | ILE | 89 | 27.514 | −0.247 | 50.243 | 1.00 | 33.35 | B |
| ATOM | 568 | CD1 | ILE | 89 | 28.486 | 0.077 | 51.357 | 1.00 | 33.52 | B |
| ATOM | 569 | C | ILE | 89 | 25.552 | 1.786 | 47.636 | 1.00 | 32.98 | B |
| ATOM | 570 | O | ILE | 89 | 24.485 | 2.243 | 48.016 | 1.00 | 33.67 | B |
| ATOM | 571 | N | LEU | 90 | 26.258 | 2.341 | 46.662 | 1.00 | 32.32 | B |
| ATOM | 572 | CA | LEU | 90 | 25.782 | 3.540 | 45.996 | 1.00 | 32.57 | B |
| ATOM | 573 | CB | LEU | 90 | 26.866 | 4.097 | 45.074 | 1.00 | 30.54 | B |
| ATOM | 574 | CG | LEU | 90 | 26.431 | 5.292 | 44.229 | 1.00 | 29.69 | B |
| ATOM | 575 | CD1 | LEU | 90 | 26.018 | 6.448 | 45.122 | 1.00 | 28.62 | B |
| ATOM | 576 | CD2 | LEU | 90 | 27.564 | 5.695 | 43.319 | 1.00 | 31.53 | B |
| ATOM | 577 | C | LEU | 90 | 24.504 | 3.272 | 45.202 | 1.00 | 32.92 | B |
| ATOM | 578 | O | LEU | 90 | 23.567 | 4.074 | 45.240 | 1.00 | 32.45 | B |
| ATOM | 579 | N | ASP | 91 | 24.466 | 2.147 | 44.491 | 1.00 | 33.45 | B |
| ATOM | 580 | CA | ASP | 91 | 23.292 | 1.785 | 43.699 | 1.00 | 34.72 | B |
| ATOM | 581 | CB | ASP | 91 | 23.520 | 0.470 | 42.940 | 1.00 | 35.65 | B |
| ATOM | 582 | CG | ASP | 91 | 24.593 | 0.582 | 41.863 | 1.00 | 39.61 | B |
| ATOM | 583 | OD1 | ASP | 91 | 24.686 | 1.648 | 41.214 | 1.00 | 40.33 | B |
| ATOM | 584 | OD2 | ASP | 91 | 25.335 | −0.409 | 41.661 | 1.00 | 41.38 | B |
| ATOM | 585 | C | ASP | 91 | 22.068 | 1.633 | 44.597 | 1.00 | 33.10 | B |
| ATOM | 586 | O | ASP | 91 | 20.954 | 1.885 | 44.174 | 1.00 | 33.56 | B |
| ATOM | 587 | N | GLU | 92 | 22.290 | 1.221 | 45.839 | 1.00 | 32.56 | B |
| ATOM | 588 | CA | GLU | 92 | 21.196 | 1.044 | 46.783 | 1.00 | 34.16 | B |
| ATOM | 589 | CB | GLU | 92 | 21.657 | 0.171 | 47.954 | 1.00 | 37.44 | B |
| ATOM | 590 | CG | GLU | 92 | 20.545 | −0.258 | 48.890 | 1.00 | 42.74 | B |
| ATOM | 591 | CD | GLU | 92 | 20.880 | −1.536 | 49.648 | 1.00 | 46.50 | B |
| ATOM | 592 | OE1 | GLU | 92 | 20.053 | −1.956 | 50.490 | 1.00 | 47.07 | B |
| ATOM | 593 | OE2 | GLU | 92 | 21.962 | −2.120 | 49.396 | 1.00 | 46.74 | B |
| ATOM | 594 | C | GLU | 92 | 20.709 | 2.409 | 47.280 | 1.00 | 32.53 | B |
| ATOM | 595 | O | GLU | 92 | 19.518 | 2.608 | 47.519 | 1.00 | 30.70 | B |
| ATOM | 596 | N | VAL | 93 | 21.641 | 3.348 | 47.422 | 1.00 | 31.20 | B |
| ATOM | 597 | CA | VAL | 93 | 21.303 | 4.699 | 47.854 | 1.00 | 31.28 | B |
| ATOM | 598 | CB | VAL | 93 | 22.580 | 5.569 | 48.076 | 1.00 | 31.49 | B |
| ATOM | 599 | CG1 | VAL | 93 | 22.194 | 7.010 | 48.365 | 1.00 | 27.40 | B |
| ATOM | 600 | CG2 | VAL | 93 | 23.398 | 5.004 | 49.233 | 1.00 | 33.28 | B |
| ATOM | 601 | C | VAL | 93 | 20.452 | 5.322 | 46.750 | 1.00 | 29.79 | B |
| ATOM | 602 | O | VAL | 93 | 19.416 | 5.913 | 47.013 | 1.00 | 28.28 | B |
| ATOM | 603 | N | ILE | 94 | 20.899 | 5.163 | 45.510 | 1.00 | 27.82 | B |
| ATOM | 604 | CA | ILE | 94 | 20.166 | 5.703 | 44.378 | 1.00 | 30.44 | B |
| ATOM | 605 | CB | ILE | 94 | 20.915 | 5.429 | 43.051 | 1.00 | 28.59 | B |
| ATOM | 606 | CG2 | ILE | 94 | 20.035 | 5.787 | 41.853 | 1.00 | 26.78 | B |
| ATOM | 607 | CG1 | ILE | 94 | 22.216 | 6.240 | 43.037 | 1.00 | 27.01 | B |
| ATOM | 608 | CD1 | ILE | 94 | 23.087 | 5.978 | 41.846 | 1.00 | 26.60 | B |
| ATOM | 609 | C | ILE | 94 | 18.749 | 5.131 | 44.306 | 1.00 | 32.32 | B |
| ATOM | 610 | O | ILE | 94 | 17.872 | 5.738 | 43.714 | 1.00 | 32.23 | B |
| ATOM | 611 | N | MET | 95 | 18.531 | 3.968 | 44.920 | 1.00 | 34.51 | B |
| ATOM | 612 | CA | MET | 95 | 17.201 | 3.360 | 44.923 | 1.00 | 36.17 | B |
| ATOM | 613 | CB | MET | 95 | 17.282 | 1.850 | 45.149 | 1.00 | 38.61 | B |
| ATOM | 614 | CG | MET | 95 | 17.372 | 1.017 | 43.881 | 1.00 | 40.44 | B |
| ATOM | 615 | SD | MET | 95 | 17.488 | −0.772 | 44.242 | 1.00 | 46.46 | B |
| ATOM | 616 | CE | MET | 95 | 19.102 | −1.171 | 43.546 | 1.00 | 44.51 | B |
| ATOM | 617 | C | MET | 95 | 16.315 | 3.979 | 45.996 | 1.00 | 36.50 | B |
| ATOM | 618 | O | MET | 95 | 15.113 | 3.732 | 46.030 | 1.00 | 37.42 | B |
| ATOM | 619 | N | GLY | 96 | 16.914 | 4.775 | 46.879 | 1.00 | 36.28 | B |
| ATOM | 620 | CA | GLY | 96 | 16.145 | 5.414 | 47.932 | 1.00 | 35.74 | B |
| ATOM | 621 | C | GLY | 96 | 16.366 | 4.830 | 49.314 | 1.00 | 36.78 | B |
| ATOM | 622 | O | GLY | 96 | 15.538 | 5.026 | 50.210 | 1.00 | 37.90 | B |
| ATOM | 623 | N | TYR | 97 | 17.479 | 4.118 | 49.487 | 1.00 | 36.85 | B |
| ATOM | 624 | CA | TYR | 97 | 17.835 | 3.496 | 50.763 | 1.00 | 37.58 | B |
| ATOM | 625 | CB | TYR | 97 | 18.381 | 2.081 | 50.525 | 1.00 | 40.65 | B |
| ATOM | 626 | CG | TYR | 97 | 17.341 | 1.025 | 50.217 | 1.00 | 45.13 | B |
| ATOM | 627 | CD1 | TYR | 97 | 16.518 | 0.518 | 51.220 | 1.00 | 46.62 | B |
| ATOM | 628 | CE1 | TYR | 97 | 15.558 | −0.454 | 50.944 | 1.00 | 49.26 | B |
| ATOM | 629 | CD2 | TYR | 97 | 17.182 | 0.533 | 48.921 | 1.00 | 46.06 | B |
| ATOM | 630 | CE2 | TYR | 97 | 16.228 | −0.436 | 48.630 | 1.00 | 49.09 | B |
| ATOM | 631 | CZ | TYR | 97 | 15.417 | −0.928 | 49.646 | 1.00 | 50.42 | B |
| ATOM | 632 | OH | TYR | 97 | 14.465 | −1.888 | 49.358 | 1.00 | 52.50 | 8 |
| ATOM | 633 | C | TYR | 97 | 18.889 | 4.304 | 51.526 | 1.00 | 35.44 | B |
| ATOM | 634 | O | TYR | 97 | 19.789 | 4.876 | 50.926 | 1.00 | 37.02 | B |
| ATOM | 635 | N | ASN | 98 | 18.776 | 4.349 | 52.849 | 1.00 | 31.97 | B |
| ATOM | 636 | CA | ASN | 98 | 19.759 | 5.059 | 53.662 | 1.00 | 30.42 | B |
| ATOM | 637 | CB | ASN | 98 | 19.169 | 5.460 | 55.025 | 1.00 | 30.64 | B |
| ATOM | 638 | CG | ASN | 98 | 18.239 | 6.663 | 54.945 | 1.00 | 28.74 | B |
| ATOM | 639 | OD1 | ASN | 98 | 18.255 | 7.413 | 53.981 | 1.00 | 29.47 | B |
| ATOM | 640 | ND2 | ASN | 98 | 17.436 | 6.855 | 55.984 | 1.00 | 27.34 | B |
| ATOM | 641 | C | ASN | 98 | 20.942 | 4.124 | 53.897 | 1.00 | 29.81 | B |
| ATOM | 642 | O | ASN | 98 | 20.762 | 3.006 | 54.324 | 1.00 | 29.82 | B |
| ATOM | 643 | N | CYS | 99 | 22.152 | 4.590 | 53.615 | 1.00 | 28.53 | B |
| ATOM | 644 | CA | CYS | 99 | 23.339 | 3.767 | 53.816 | 1.00 | 26.90 | B |
| ATOM | 645 | CB | CYS | 99 | 23.974 | 3.384 | 52.477 | 1.00 | 28.87 | B |
| ATOM | 646 | SG | CYS | 99 | 22.946 | 2.349 | 51.428 | 1.00 | 34.21 | B |
| ATOM | 647 | C | CYS | 99 | 24.382 | 4.465 | 54.677 | 1.00 | 25.00 | B |
| ATOM | 648 | O | CYS | 99 | 24.380 | 5.670 | 54.830 | 1.00 | 25.25 | B |
| ATOM | 649 | N | THR | 100 | 25.285 | 3.671 | 55.232 | 1.00 | 23.32 | B |
| ATOM | 650 | CA | THR | 100 | 26.341 | 4.187 | 56.080 | 1.00 | 19.59 | B |
| ATOM | 651 | CB | THR | 100 | 25.876 | 4.258 | 57.544 | 1.00 | 17.10 | B |
| ATOM | 652 | OG1 | THR | 100 | 24.789 | 5.179 | 57.657 | 1.00 | 16.21 | B |
| ATOM | 653 | CG2 | THR | 100 | 27.005 | 4.696 | 58.456 | 1.00 | 15.27 | B |
| ATOM | 654 | C | THR | 100 | 27.552 | 3.266 | 55.982 | 1.00 | 21.18 | B |
| ATOM | 655 | O | THR | 100 | 27.417 | 2.039 | 56.005 | 1.00 | 22.70 | B |
| ATOM | 656 | N | ILE | 101 | 28.732 | 3.858 | 55.849 | 1.00 | 18.53 | B |
| ATOM | 657 | CA | ILE | 101 | 29.967 | 3.097 | 55.782 | 1.00 | 17.55 | B |
| ATOM | 658 | CB | ILE | 101 | 30.650 | 3.212 | 54.420 | 1.00 | 16.14 | B |
| ATOM | 659 | CG2 | ILE | 101 | 31.939 | 2.414 | 54.423 | 1.00 | 16.50 | B |
| ATOM | 660 | CG1 | ILE | 101 | 29.730 | 2.690 | 53.318 | 1.00 | 14.57 | B |
| ATOM | 661 | CD1 | ILE | 101 | 30.186 | 3.077 | 51.930 | 1.00 | 14.45 | B |
| ATOM | 662 | C | ILE | 101 | 30.913 | 3.654 | 56.834 | 1.00 | 19.99 | B |
| ATOM | 663 | O | ILE | 101 | 31.296 | 4.822 | 56.786 | 1.00 | 20.78 | B |
| ATOM | 664 | N | PHE | 102 | 31.273 | 2.808 | 57.793 | 1.00 | 19.14 | B |
| ATOM | 665 | CA | PHE | 102 | 32.176 | 3.179 | 58.876 | 1.00 | 17.58 | B |
| ATOM | 666 | CB | PHE | 102 | 31.835 | 2.373 | 60.123 | 1.00 | 17.67 | B |
| ATOM | 667 | CG | PHE | 102 | 30.618 | 2.842 | 60.847 | 1.00 | 17.05 | B |
| ATOM | 668 | CD1 | PHE | 102 | 30.714 | 3.855 | 61.790 | 1.00 | 16.04 | B |
| ATOM | 669 | CD2 | PHE | 102 | 29.386 | 2.239 | 60.624 | 1.00 | 16.40 | B |
| ATOM | 670 | CE1 | PHE | 102 | 29.603 | 4.265 | 62.508 | 1.00 | 16.56 | B |
| ATOM | 671 | CE2 | PHE | 102 | 28.268 | 2.643 | 61.337 | 1.00 | 18.62 | B |
| ATOM | 672 | CZ | PHE | 102 | 28.377 | 3.658 | 62.283 | 1.00 | 16.81 | B |
| ATOM | 673 | C | PHE | 102 | 33.625 | 2.891 | 58.515 | 1.00 | 16.69 | B |
| ATOM | 674 | O | PHE | 102 | 33.910 | 2.289 | 57.516 | 1.00 | 18.17 | B |
| ATOM | 675 | N | ALA | 103 | 34.535 | 3.338 | 59.366 | 1.00 | 17.68 | B |
| ATOM | 676 | CA | ALA | 103 | 35.961 | 3.089 | 59.187 | 1.00 | 17.02 | B |
| ATOM | 677 | CB | ALA | 103 | 36.620 | 4.229 | 58.451 | 1.00 | 16.82 | B |
| ATOM | 678 | C | ALA | 103 | 36.471 | 2.991 | 60.617 | 1.00 | 17.64 | B |
| ATOM | 679 | O | ALA | 103 | 36.482 | 3.963 | 61.339 | 1.00 | 18.79 | B |
| ATOM | 680 | N | TYR | 104 | 36.866 | 1.786 | 61.012 | 1.00 | 18.22 | B |
| ATOM | 681 | CA | TYR | 104 | 37.340 | 1.540 | 62.368 | 1.00 | 16.40 | B |
| ATOM | 682 | CB | TYR | 104 | 36.436 | 0.496 | 63.034 | 1.00 | 15.83 | B |
| ATOM | 683 | CG | TYR | 104 | 36.706 | 0.291 | 64.508 | 1.00 | 12.67 | B |
| ATOM | 684 | CD1 | TYR | 104 | 37.771 | −0.501 | 64.941 | 1.00 | 10.95 | B |
| ATOM | 685 | CE1 | TYR | 104 | 38.046 | −0.659 | 66.301 | 1.00 | 11.52 | B |
| ATOM | 686 | CD2 | TYR | 104 | 35.919 | 0.920 | 65.469 | 1.00 | 10.91 | B |
| ATOM | 687 | CE2 | TYR | 104 | 36.187 | 0.768 | 66.832 | 1.00 | 12.42 | B |
| ATOM | 688 | CZ | TYR | 104 | 37.253 | −0.023 | 67.239 | 1.00 | 10.32 | B |
| ATOM | 689 | OH | TYR | 104 | 37.526 | −0.180 | 68.574 | 1.00 | 11.99 | B |
| ATOM | 690 | C | TYR | 104 | 38.778 | 1.061 | 62.380 | 1.00 | 15.64 | B |
| ATOM | 691 | O | TYR | 104 | 39.203 | 0.348 | 61.497 | 1.00 | 17.51 | B |
| ATOM | 692 | N | GLY | 105 | 39.524 | 1.456 | 63.397 | 1.00 | 15.78 | B |
| ATOM | 693 | CA | GLY | 105 | 40.904 | 1.047 | 63.475 | 1.00 | 16.05 | B |
| ATOM | 694 | C | GLY | 105 | 41.748 | 2.044 | 64.226 | 1.00 | 16.81 | B |
| ATOM | 695 | O | GLY | 105 | 41.318 | 3.151 | 64.526 | 1.00 | 19.22 | B |
| ATOM | 696 | N | GLN | 106 | 42.963 | 1.616 | 64.531 | 1.00 | 18.16 | B |
| ATOM | 697 | CA | GLN | 106 | 43.940 | 2.408 | 65.244 | 1.00 | 18.74 | B |
| ATOM | 698 | CB | GLN | 106 | 45.122 | 1.519 | 65.652 | 1.00 | 19.69 | B |
| ATOM | 699 | CG | GLN | 106 | 46.278 | 2.251 | 66.305 | 1.00 | 23.87 | B |
| ATOM | 700 | CD | GLN | 106 | 47.527 | 1.411 | 66.407 | 1.00 | 24.14 | B |
| ATOM | 701 | OE1 | GLN | 106 | 47.865 | 0.669 | 65.490 | 1.00 | 27.37 | B |
| ATOM | 702 | NE2 | GLN | 106 | 48.225 | 1.528 | 67.525 | 1.00 | 25.29 | B |
| ATOM | 703 | C | GLN | 106 | 44.440 | 3.552 | 64.363 | 1.00 | 20.10 | B |
| ATOM | 704 | O | GLN | 106 | 44.438 | 3.451 | 63.134 | 1.00 | 19.09 | B |
| ATOM | 705 | N | THR | 107 | 44.864 | 4.639 | 65.004 | 1.00 | 19.11 | B |
| ATOM | 706 | CA | THR | 107 | 45.385 | 5.792 | 64.291 | 1.00 | 18.65 | B |
| ATOM | 707 | CB | THR | 107 | 45.849 | 6.914 | 65.270 | 1.00 | 20.97 | B |
| ATOM | 708 | OG1 | THR | 107 | 44.730 | 7.405 | 66.017 | 1.00 | 19.66 | B |
| ATOM | 709 | CG2 | THR | 107 | 46.476 | 8.064 | 64.497 | 1.00 | 15.96 | B |
| ATOM | 710 | C | THR | 107 | 46.588 | 5.391 | 63.439 | 1.00 | 17.71 | B |
| ATOM | 711 | O | THR | 107 | 47.518 | 4.747 | 63.921 | 1.00 | 16.56 | B |
| ATOM | 712 | N | GLY | 108 | 46.554 | 5.786 | 62.171 | 1.00 | 17.28 | B |
| ATOM | 713 | CA | GLY | 108 | 47.642 | 5.483 | 61.267 | 1.00 | 15.71 | B |
| ATOM | 714 | C | GLY | 108 | 47.499 | 4.181 | 60.505 | 1.00 | 17.55 | B |
| ATOM | 715 | O | GLY | 108 | 48.489 | 3.682 | 59.938 | 1.00 | 17.87 | B |
| ATOM | 716 | N | THR | 109 | 46.288 | 3.626 | 60.478 | 1.00 | 15.83 | B |
| ATOM | 717 | CA | THR | 109 | 46.064 | 2.374 | 59.765 | 1.00 | 14.74 | B |
| ATOM | 718 | CB | THR | 109 | 45.276 | 1.352 | 60.632 | 1.00 | 13.57 | B |
| ATOM | 719 | OG1 | THR | 109 | 43.978 | 1.866 | 60.943 | 1.00 | 13.63 | B |
| ATOM | 720 | CG2 | THR | 109 | 46.035 | 1.064 | 61.934 | 1.00 | 12.00 | B |
| ATOM | 721 | C | THR | 109 | 45.350 | 2.573 | 58.435 | 1.00 | 15.88 | B |
| ATOM | 722 | O | THR | 109 | 45.132 | 1.602 | 57.708 | 1.00 | 14.55 | B |
| ATOM | 723 | N | GLY | 110 | 44.977 | 3.819 | 58.124 | 1.00 | 13.70 | B |
| ATOM | 724 | CA | GLY | 110 | 44.321 | 4.073 | 56.849 | 1.00 | 10.56 | B |
| ATOM | 725 | C | GLY | 110 | 42.846 | 4.433 | 56.833 | 1.00 | 10.76 | B |
| ATOM | 726 | O | GLY | 110 | 42.201 | 4.298 | 55.792 | 1.00 | 9.95 | B |
| ATOM | 727 | N | LYS | 111 | 42.302 | 4.885 | 57.959 | 1.00 | 8.99 | B |
| ATOM | 728 | CA | LYS | 111 | 40.889 | 5.267 | 58.022 | 1.00 | 11.48 | B |
| ATOM | 729 | CB | LYS | 111 | 40.497 | 5.693 | 59.449 | 1.00 | 12.59 | B |
| ATOM | 730 | CG | LYS | 111 | 40.315 | 4.531 | 60.426 | 1.00 | 15.28 | B |
| ATOM | 731 | CD | LYS | 111 | 39.651 | 4.955 | 61.738 | 1.00 | 12.73 | B |
| ATOM | 732 | CE | LYS | 111 | 40.439 | 6.034 | 62.455 | 1.00 | 11.56 | B |
| ATOM | 733 | NZ | LYS | 111 | 41.905 | 5.766 | 62.396 | 1.00 | 10.51 | B |
| ATOM | 734 | C | LYS | 111 | 40.575 | 6.408 | 57.062 | 1.00 | 13.97 | B |
| ATOM | 735 | O | LYS | 111 | 39.683 | 6.302 | 56.206 | 1.00 | 15.37 | B |
| ATOM | 736 | N | THR | 112 | 41.321 | 7.498 | 57.198 | 1.00 | 13.82 | B |
| ATOM | 737 | CA | THR | 112 | 41.120 | 8.663 | 56.353 | 1.00 | 12.58 | B |
| ATOM | 738 | CB | THR | 112 | 41.895 | 9.871 | 56.926 | 1.00 | 12.79 | B |
| ATOM | 739 | OG1 | THR | 112 | 41.408 | 10.160 | 58.245 | 1.00 | 9.63 | B |
| ATOM | 740 | CG2 | THR | 112 | 41.723 | 11.103 | 56.037 | 1.00 | 10.46 | B |
| ATOM | 741 | C | THR | 112 | 41.535 | 8.396 | 54.905 | 1.00 | 14.40 | B |
| ATOM | 742 | O | THR | 112 | 40.886 | 8.846 | 53.978 | 1.00 | 15.19 | B |
| ATOM | 743 | N | PHE | 113 | 42.618 | 7.651 | 54.723 | 1.00 | 15.74 | B |
| ATOM | 744 | CA | PHE | 113 | 43.095 | 7.326 | 53.384 | 1.00 | 17.09 | B |
| ATOM | 745 | CB | PHE | 113 | 44.316 | 6.408 | 53.463 | 1.00 | 17.69 | B |
| ATOM | 746 | CG | PHE | 113 | 44.867 | 6.030 | 52.123 | 1.00 | 20.87 | B |
| ATOM | 747 | CD1 | PHE | 113 | 45.783 | 6.849 | 51.475 | 1.00 | 22.41 | B |
| ATOM | 748 | CD2 | PHE | 113 | 44.445 | 4.871 | 51.490 | 1.00 | 22.63 | B |
| ATOM | 749 | CE1 | PHE | 113 | 46.271 | 6.517 | 50.218 | 1.00 | 22.81 | B |
| ATOM | 750 | CE2 | PHE | 113 | 44.924 | 4.529 | 50.228 | 1.00 | 23.87 | B |
| ATOM | 751 | CZ | PHE | 113 | 45.840 | 5.354 | 49.590 | 1.00 | 25.27 | B |
| ATOM | 752 | C | PHE | 113 | 42.000 | 6.626 | 52.580 | 1.00 | 18.62 | B |
| ATOM | 753 | O | PHE | 113 | 41.817 | 6.888 | 51.389 | 1.00 | 17.60 | B |
| ATOM | 754 | N | THR | 114 | 41.291 | 5.719 | 53.247 | 1.00 | 19.63 | B |
| ATOM | 755 | CA | THR | 114 | 40.212 | 4.945 | 52.646 | 1.00 | 18.57 | B |
| ATOM | 756 | CB | THR | 114 | 39.816 | 3.760 | 53.582 | 1.00 | 20.30 | B |
| ATOM | 757 | OG1 | THR | 114 | 40.970 | 2.947 | 53.828 | 1.00 | 18.79 | B |
| ATOM | 758 | CG2 | THR | 114 | 38.700 | 2.910 | 52.972 | 1.00 | 12.74 | B |
| ATOM | 759 | C | THR | 114 | 38.991 | 5.825 | 52.410 | 1.00 | 19.70 | B |
| ATOM | 760 | O | THR | 114 | 38.497 | 5.932 | 51.297 | 1.00 | 22.13 | B |
| ATOM | 761 | N | MET | 115 | 38.518 | 6.473 | 53.465 | 1.00 | 19.43 | B |
| ATOM | 762 | CA | MET | 115 | 37.345 | 7.318 | 53.347 | 1.00 | 20.55 | B |
| ATOM | 763 | CB | MET | 115 | 36.877 | 7.771 | 54.730 | 1.00 | 21.97 | B |
| ATOM | 764 | CG | MET | 115 | 36.471 | 6.620 | 55.644 | 1.00 | 27.07 | B |
| ATOM | 765 | SD | MET | 115 | 35.328 | 5.432 | 54.848 | 1.00 | 29.66 | B |
| ATOM | 766 | CE | MET | 115 | 33.753 | 6.265 | 55.089 | 1.00 | 27.98 | B |
| ATOM | 767 | C | MET | 115 | 37.532 | 8.528 | 52.454 | 1.00 | 21.26 | B |
| ATOM | 768 | O | MET | 115 | 36.639 | 8.866 | 51.674 | 1.00 | 23.74 | B |
| ATOM | 769 | N | GLU | 116 | 38.687 | 9.179 | 52.549 | 1.00 | 20.10 | B |
| ATOM | 770 | CA | GLU | 116 | 38.937 | 10.377 | 51.749 | 1.00 | 20.30 | B |
| ATOM | 771 | CB | GLU | 116 | 39.323 | 11.541 | 52.659 | 1.00 | 19.03 | B |
| ATOM | 772 | CG | GLU | 116 | 38.309 | 11.824 | 53.741 | 1.00 | 17.09 | B |
| ATOM | 773 | CD | GLU | 116 | 38.746 | 12.922 | 54.687 | 1.00 | 18.90 | B |
| ATOM | 774 | OE1 | GLU | 116 | 39.886 | 13.421 | 54.550 | 1.00 | 21.39 | B |
| ATOM | 775 | OE2 | GLU | 116 | 37.951 | 13.280 | 55.579 | 1.00 | 17.52 | B |
| ATOM | 776 | C | GLU | 116 | 40.010 | 10.194 | 50.694 | 1.00 | 20.60 | B |
| ATOM | 777 | O | GLU | 116 | 39.804 | 10.494 | 49.527 | 1.00 | 19.26 | B |
| ATOM | 778 | N | GLY | 117 | 41.166 | 9.708 | 51.116 | 1.00 | 22.39 | B |
| ATOM | 779 | CA | GLY | 117 | 42.249 | 9.508 | 50.176 | 1.00 | 24.67 | B |
| ATOM | 780 | C | GLY | 117 | 43.194 | 10.689 | 50.144 | 1.00 | 25.76 | B |
| ATOM | 781 | O | GLY | 117 | 43.056 | 11.630 | 50.918 | 1.00 | 24.17 | B |
| ATOM | 782 | N | GLU | 118 | 44.162 | 10.635 | 49.237 | 1.00 | 27.49 | B |
| ATOM | 783 | CA | GLU | 118 | 45.133 | 11.710 | 49.128 | 1.00 | 28.73 | B |
| ATOM | 784 | CB | GLU | 118 | 46.465 | 11.273 | 49.740 | 1.00 | 30.64 | B |
| ATOM | 785 | CG | GLU | 118 | 46.311 | 10.255 | 50.853 | 1.00 | 35.23 | B |
| ATOM | 786 | CD | GLU | 118 | 47.579 | 10.060 | 51.657 | 1.00 | 37.43 | B |
| ATOM | 787 | OE1 | GLU | 118 | 48.671 | 9.993 | 51.049 | 1.00 | 35.58 | B |
| ATOM | 788 | OE2 | GLU | 118 | 47.476 | 9.958 | 52.900 | 1.00 | 40.04 | B |
| ATOM | 789 | C | GLU | 118 | 45.338 | 12.082 | 47.671 | 1.00 | 27.97 | B |
| ATOM | 790 | O | GLU | 118 | 44.692 | 11.542 | 46.779 | 1.00 | 29.50 | B |
| ATOM | 791 | N | ARG | 119 | 46.244 | 13.017 | 47.436 | 1.00 | 25.87 | B |
| ATOM | 792 | CA | ARG | 119 | 46.532 | 13.439 | 46.085 | 1.00 | 25.52 | B |
| ATOM | 793 | CB | ARG | 119 | 46.613 | 14.968 | 46.006 | 1.00 | 24.48 | B |
| ATOM | 794 | CG | ARG | 119 | 45.323 | 15.708 | 46.358 | 1.00 | 23.62 | B |
| ATOM | 795 | CD | ARG | 119 | 44.190 | 15.361 | 45.387 | 1.00 | 22.16 | B |
| ATOM | 796 | NE | ARG | 119 | 44.654 | 15.191 | 44.011 | 1.00 | 20.25 | B |
| ATOM | 797 | CZ | ARG | 119 | 44.382 | 16.018 | 43.005 | 1.00 | 19.31 | B |
| ATOM | 798 | NH1 | ARG | 119 | 43.642 | 17.102 | 43.203 | 1.00 | 19.24 | B |
| ATOM | 799 | NH2 | ARG | 119 | 44.842 | 15.744 | 41.791 | 1.00 | 17.50 | B |
| ATOM | 800 | C | ARG | 119 | 47.857 | 12.836 | 45.654 | 1.00 | 26.80 | B |
| ATOM | 801 | O | ARG | 119 | 48.779 | 12.711 | 46.457 | 1.00 | 25.89 | B |
| ATOM | 802 | N | SER | 120 | 47.942 | 12.440 | 44.390 | 1.00 | 25.98 | B |
| ATOM | 803 | CA | SER | 120 | 49.189 | 11.893 | 43.880 | 1.00 | 28.78 | B |
| ATOM | 804 | CB | SER | 120 | 49.015 | 11.326 | 42.472 | 1.00 | 29.79 | B |
| ATOM | 805 | OG | SER | 120 | 48.428 | 10.038 | 42.508 | 1.00 | 33.26 | B |
| ATOM | 806 | C | SER | 120 | 50.130 | 13.077 | 43.834 | 1.00 | 27.18 | B |
| ATOM | 807 | O | SER | 120 | 49.779 | 14.121 | 43.326 | 1.00 | 27.97 | B |
| ATOM | 808 | N | PRO | 121 | 51.348 | 12.913 | 44.357 | 1.00 | 27.06 | B |
| ATOM | 809 | CD | PRO | 121 | 51.902 | 11.662 | 44.900 | 1.00 | 26.17 | B |
| ATOM | 810 | CA | PRO | 121 | 52.350 | 13.987 | 44.381 | 1.00 | 27.66 | B |
| ATOM | 811 | CB | PRO | 121 | 53.528 | 13.342 | 45.117 | 1.00 | 27.55 | B |
| ATOM | 812 | CG | PRO | 121 | 53.386 | 11.899 | 44.779 | 1.00 | 28.94 | B |
| ATOM | 813 | C | PRO | 121 | 52.760 | 14.591 | 43.031 | 1.00 | 27.47 | B |
| ATOM | 814 | O | PRO | 121 | 52.773 | 13.914 | 42.009 | 1.00 | 27.14 | B |
| ATOM | 815 | N | ASN | 122 | 53.072 | 15.885 | 43.050 | 1.00 | 27.34 | B |
| ATOM | 816 | CA | ASN | 122 | 53.517 | 16.615 | 41.865 | 1.00 | 28.41 | B |
| ATOM | 817 | CB | ASN | 122 | 54.690 | 15.875 | 41.217 | 1.00 | 29.21 | B |
| ATOM | 818 | CG | ASN | 122 | 55.857 | 16.789 | 40.906 | 1.00 | 29.30 | B |
| ATOM | 819 | OD1 | ASN | 122 | 56.355 | 17.491 | 41.777 | 1.00 | 30.37 | B |
| ATOM | 820 | ND2 | ASN | 122 | 56.305 | 16.774 | 39.656 | 1.00 | 30.61 | B |
| ATOM | 821 | C | ASN | 122 | 52.434 | 16.859 | 40.817 | 1.00 | 28.67 | B |
| ATOM | 822 | O | ASN | 122 | 52.725 | 16.940 | 39.627 | 1.00 | 25.87 | B |
| ATOM | 823 | N | GLU | 123 | 51.191 | 16.985 | 41.265 | 1.00 | 30.12 | B |
| ATOM | 824 | CA | GLU | 123 | 50.070 | 17.240 | 40.356 | 1.00 | 33.32 | B |
| ATOM | 825 | CB | GLU | 123 | 50.105 | 18.699 | 39.870 | 1.00 | 33.54 | B |
| ATOM | 826 | CG | GLU | 123 | 50.037 | 19.748 | 40.968 | 1.00 | 33.76 | B |
| ATOM | 827 | CD | GLU | 123 | 49.872 | 21.158 | 40.420 | 1.00 | 34.11 | B |
| ATOM | 828 | OE1 | GLU | 123 | 50.763 | 21.623 | 39.678 | 1.00 | 32.71 | B |
| ATOM | 829 | OE2 | GLU | 123 | 48.848 | 21.804 | 40.734 | 1.00 | 33.32 | B |
| ATOM | 830 | C | GLU | 123 | 50.061 | 16.307 | 39.137 | 1.00 | 34.30 | B |
| ATOM | 831 | O | GLU | 123 | 49.856 | 16.743 | 38.013 | 1.00 | 32.10 | B |
| ATOM | 832 | N | GLU | 124 | 50.283 | 15.020 | 39.373 | 1.00 | 36.35 | B |
| ATOM | 833 | CA | GLU | 124 | 50.303 | 14.046 | 38.292 | 1.00 | 36.52 | B |
| ATOM | 834 | CB | GLU | 124 | 50.709 | 12.678 | 38.846 | 1.00 | 40.35 | B |
| ATOM | 835 | CG | GLU | 124 | 51.279 | 11.711 | 37.815 | 1.00 | 45.05 | B |
| ATOM | 836 | CD | GLU | 124 | 52.026 | 10.550 | 38.458 | 1.00 | 47.77 | B |
| ATOM | 837 | OE1 | GLU | 124 | 51.966 | 10.427 | 39.705 | 1.00 | 47.83 | B |
| ATOM | 838 | OE2 | GLU | 124 | 52.671 | 9.769 | 37.720 | 1.00 | 48.04 | B |
| ATOM | 839 | C | GLU | 124 | 48.942 | 13.964 | 37.590 | 1.00 | 36.15 | B |
| ATOM | 840 | O | GLU | 124 | 48.876 | 13.987 | 36.363 | 1.00 | 34.16 | B |
| ATOM | 841 | N | TYR | 125 | 47.859 | 13.886 | 38.361 | 1.00 | 35.31 | B |
| ATOM | 842 | CA | TYR | 125 | 46.524 | 13.803 | 37.770 | 1.00 | 36.12 | B |
| ATOM | 843 | CB | TYR | 125 | 45.863 | 12.440 | 38.054 | 1.00 | 38.61 | B |
| ATOM | 844 | CG | TYR | 125 | 46.757 | 11.216 | 37.992 | 1.00 | 39.31 | B |
| ATOM | 845 | CD1 | TYR | 125 | 47.657 | 10.933 | 39.019 | 1.00 | 39.77 | B |
| ATOM | 846 | CE1 | TYR | 125 | 48.454 | 9.784 | 38.987 | 1.00 | 40.96 | B |
| ATOM | 847 | CD2 | TYR | 125 | 46.675 | 10.321 | 36.922 | 1.00 | 39.64 | B |
| ATOM | 848 | CE2 | TYR | 125 | 47.468 | 9.169 | 36.879 | 1.00 | 40.42 | B |
| ATOM | 849 | CZ | TYR | 125 | 48.355 | 8.908 | 37.916 | 1.00 | 41.60 | B |
| ATOM | 850 | OH | TYR | 125 | 49.141 | 7.776 | 37.882 | 1.00 | 43.64 | B |
| ATOM | 851 | C | TYR | 125 | 45.590 | 14.873 | 38.332 | 1.00 | 35.75 | B |
| ATOM | 852 | O | TYR | 125 | 45.925 | 15.577 | 39.273 | 1.00 | 36.04 | B |
| ATOM | 853 | N | THR | 126 | 44.409 | 14.976 | 37.729 | 1.00 | 35.01 | B |
| ATOM | 854 | CA | THR | 126 | 43.385 | 15.901 | 38.189 | 1.00 | 34.12 | B |
| ATOM | 855 | CB | THR | 126 | 42.393 | 16.275 | 37.064 | 1.00 | 34.09 | B |
| ATOM | 856 | OG1 | THR | 126 | 41.885 | 15.080 | 36.458 | 1.00 | 36.33 | B |
| ATOM | 857 | CG2 | THR | 126 | 43.075 | 17.134 | 36.005 | 1.00 | 30.16 | B |
| ATOM | 858 | C | THR | 126 | 42.645 | 15.117 | 39.271 | 1.00 | 34.15 | B |
| ATOM | 859 | O | THR | 126 | 42.555 | 13.896 | 39.197 | 1.00 | 35.30 | B |
| ATOM | 860 | N | TRP | 127 | 42.111 | 15.807 | 40.270 | 1.00 | 33.25 | B |
| ATOM | 861 | CA | TRP | 127 | 41.422 | 15.133 | 41.363 | 1.00 | 31.64 | B |
| ATOM | 862 | CB | TRP | 127 | 40.596 | 16.135 | 42.182 | 1.00 | 28.58 | B |
| ATOM | 863 | CG | TRP | 127 | 39.362 | 16.610 | 41.489 | 1.00 | 25.55 | B |
| ATOM | 864 | CD2 | TRP | 127 | 38.066 | 16.008 | 41.551 | 1.00 | 23.28 | B |
| ATOM | 865 | CE2 | TRP | 127 | 37.218 | 16.754 | 40.699 | 1.00 | 23.64 | B |
| ATOM | 866 | CE3 | TRP | 127 | 37.537 | 14.907 | 42.244 | 1.00 | 23.43 | B |
| ATOM | 867 | CD1 | TRP | 127 | 39.255 | 17.667 | 40.631 | 1.00 | 23.80 | B |
| ATOM | 868 | NE1 | TRP | 127 | 37.969 | 17.761 | 40.150 | 1.00 | 24.71 | B |
| ATOM | 869 | CZ2 | TRP | 127 | 35.867 | 16.433 | 40.518 | 1.00 | 24.05 | B |
| ATOM | 870 | CZ3 | TRP | 127 | 36.192 | 14.585 | 42.065 | 1.00 | 24.74 | B |
| ATOM | 871 | CH2 | TRP | 127 | 35.372 | 15.351 | 41.207 | 1.00 | 26.04 | B |
| ATOM | 872 | C | TRP | 127 | 40.522 | 13.968 | 40.931 | 1.00 | 31.94 | B |
| ATOM | 873 | O | TRP | 127 | 40.510 | 12.927 | 41.579 | 1.00 | 32.64 | B |
| ATOM | 874 | N | GLU | 128 | 39.781 | 14.131 | 39.838 | 1.00 | 32.66 | B |
| ATOM | 875 | CA | GLU | 128 | 38.869 | 13.078 | 39.394 | 1.00 | 33.32 | B |
| ATOM | 876 | CB | GLU | 128 | 37.785 | 13.669 | 38.502 | 1.00 | 34.68 | B |
| ATOM | 877 | CG | GLU | 128 | 38.287 | 14.201 | 37.178 | 1.00 | 39.01 | B |
| ATOM | 878 | CD | GLU | 128 | 37.206 | 14.964 | 36.442 | 1.00 | 42.74 | B |
| ATOM | 879 | OE1 | GLU | 128 | 36.895 | 16.100 | 36.867 | 1.00 | 44.33 | B |
| ATOM | 880 | OE2 | GLU | 128 | 36.654 | 14.422 | 35.458 | 1.00 | 43.63 | B |
| ATOM | 881 | C | GLU | 128 | 39.512 | 11.879 | 38.700 | 1.00 | 32.67 | B |
| ATOM | 882 | O | GLU | 128 | 38.825 | 10.930 | 38.348 | 1.00 | 31.45 | B |
| ATOM | 883 | N | GLU | 129 | 40.825 | 11.926 | 38.500 | 1.00 | 32.62 | B |
| ATOM | 884 | CA | GLU | 129 | 41.532 | 10.815 | 37.871 | 1.00 | 33.28 | B |
| ATOM | 885 | CB | GLU | 129 | 42.192 | 11.246 | 36.561 | 1.00 | 35.75 | B |
| ATOM | 886 | CG | GLU | 129 | 41.218 | 11.496 | 35.420 | 1.00 | 39.64 | B |
| ATOM | 887 | CD | GLU | 129 | 41.922 | 11.680 | 34.082 | 1.00 | 42.49 | B |
| ATOM | 888 | OE1 | GLU | 129 | 41.266 | 12.139 | 33.119 | 1.00 | 43.56 | B |
| ATOM | 889 | OE2 | GLU | 129 | 43.129 | 11.367 | 33.996 | 1.00 | 45.44 | B |
| ATOM | 890 | C | GLU | 129 | 42.602 | 10.280 | 38.808 | 1.00 | 33.23 | B |
| ATOM | 891 | O | GLU | 129 | 43.242 | 9.297 | 38.511 | 1.00 | 33.33 | B |
| ATOM | 892 | N | ASP | 130 | 42.776 | 10.934 | 39.951 | 1.00 | 32.98 | B |
| ATOM | 893 | CA | ASP | 130 | 43.789 | 10.516 | 40.912 | 1.00 | 32.86 | B |
| ATOM | 894 | CB | ASP | 130 | 43.884 | 11.544 | 42.045 | 1.00 | 34.15 | B |
| ATOM | 895 | CG | ASP | 130 | 45.247 | 11.564 | 42.699 | 1.00 | 35.32 | B |
| ATOM | 896 | OD1 | ASP | 130 | 45.765 | 10.477 | 43.030 | 1.00 | 36.91 | B |
| ATOM | 897 | OD2 | ASP | 130 | 45.801 | 12.665 | 42.882 | 1.00 | 36.83 | B |
| ATOM | 898 | C | ASP | 130 | 43.468 | 9.129 | 41.485 | 1.00 | 33.07 | B |
| ATOM | 899 | O | ASP | 130 | 42.429 | 8.928 | 42.114 | 1.00 | 32.52 | B |
| ATOM | 900 | N | PRO | 131 | 44.367 | 8.152 | 41.268 | 1.00 | 32.43 | B |
| ATOM | 901 | CD | PRO | 131 | 45.638 | 8.278 | 40.533 | 1.00 | 32.63 | B |
| ATOM | 902 | CA | PRO | 131 | 44.186 | 6.782 | 41.757 | 1.00 | 30.77 | B |
| ATOM | 903 | CB | PRO | 131 | 45.339 | 6.029 | 41.102 | 1.00 | 31.15 | B |
| ATOM | 904 | CG | PRO | 131 | 46.399 | 7.073 | 41.005 | 1.00 | 31.37 | B |
| ATOM | 905 | C | PRO | 131 | 44.192 | 6.673 | 43.283 | 1.00 | 30.54 | B |
| ATOM | 906 | O | PRO | 131 | 43.717 | 5.688 | 43.845 | 1.00 | 31.07 | B |
| ATOM | 907 | N | LEU | 132 | 44.721 | 7.691 | 43.953 | 1.00 | 28.68 | B |
| ATOM | 908 | CA | LEU | 132 | 44.750 | 7.684 | 45.407 | 1.00 | 26.49 | B |
| ATOM | 909 | CB | LEU | 132 | 45.965 | 8.461 | 45.918 | 1.00 | 24.68 | B |
| ATOM | 910 | CG | LEU | 132 | 47.355 | 7.961 | 45.497 | 1.00 | 25.57 | B |
| ATOM | 911 | CD1 | LEU | 132 | 48.414 | 8.782 | 46.221 | 1.00 | 24.29 | B |
| ATOM | 912 | CD2 | LEU | 132 | 47.526 | 6.481 | 45.843 | 1.00 | 26.94 | B |
| ATOM | 913 | C | LEU | 132 | 43.455 | 8.248 | 46.008 | 1.00 | 26.30 | B |
| ATOM | 914 | O | LEU | 132 | 43.294 | 8.285 | 47.228 | 1.00 | 26.84 | B |
| ATOM | 915 | N | ALA | 133 | 42.532 | 8.672 | 45.145 | 1.00 | 24.55 | B |
| ATOM | 916 | CA | ALA | 133 | 41.243 | 9.217 | 45.572 | 1.00 | 25.15 | B |
| ATOM | 917 | CB | ALA | 133 | 40.393 | 9.562 | 44.352 | 1.00 | 24.26 | B |
| ATOM | 918 | C | ALA | 133 | 40.502 | 8.215 | 46.453 | 1.00 | 25.64 | B |
| ATOM | 919 | O | ALA | 133 | 40.528 | 7.034 | 46.201 | 1.00 | 27.86 | B |
| ATOM | 920 | N | GLY | 134 | 39.831 | 8.706 | 47.485 | 1.00 | 26.27 | B |
| ATOM | 921 | CA | GLY | 134 | 39.107 | 7.822 | 48.379 | 1.00 | 24.63 | B |
| ATOM | 922 | C | GLY | 134 | 37.633 | 7.705 | 48.038 | 1.00 | 24.63 | B |
| ATOM | 923 | O | GLY | 134 | 37.176 | 8.224 | 47.013 | 1.00 | 23.91 | B |
| ATOM | 924 | N | ILE | 135 | 36.887 | 7.030 | 48.910 | 1.00 | 22.69 | B |
| ATOM | 925 | CA | ILE | 135 | 35.457 | 6.816 | 48.704 | 1.00 | 21.86 | B |
| ATOM | 926 | CB | ILE | 135 | 34.839 | 6.028 | 49.898 | 1.00 | 21.68 | B |
| ATOM | 927 | CG2 | ILE | 135 | 33.315 | 5.945 | 49.745 | 1.00 | 20.01 | B |
| ATOM | 928 | CG1 | ILE | 135 | 35.464 | 4.628 | 49.971 | 1.00 | 20.31 | B |
| ATOM | 929 | CD1 | ILE | 135 | 35.183 | 3.865 | 51.246 | 1.00 | 16.89 | B |
| ATOM | 930 | C | ILE | 135 | 34.652 | 8.103 | 48.481 | 1.00 | 20.87 | B |
| ATOM | 931 | O | ILE | 135 | 33.956 | 8.228 | 47.495 | 1.00 | 19.45 | B |
| ATOM | 932 | N | ILE | 136 | 34.762 | 9.053 | 49.405 | 1.00 | 20.74 | B |
| ATOM | 933 | CA | ILE | 136 | 34.018 | 10.309 | 49.297 | 1.00 | 19.78 | B |
| ATOM | 934 | CB | ILE | 136 | 34.420 | 11.273 | 50.436 | 1.00 | 19.46 | B |
| ATOM | 935 | CG2 | ILE | 136 | 33.654 | 12.581 | 50.302 | 1.00 | 23.46 | B |
| ATOM | 936 | CG1 | ILE | 136 | 34.128 | 10.616 | 51.792 | 1.00 | 19.18 | B |
| ATOM | 937 | CD1 | ILE | 136 | 34.597 | 11.398 | 53.011 | 1.00 | 20.13 | B |
| ATOM | 938 | C | ILE | 136 | 34.146 | 11.016 | 47.929 | 1.00 | 19.32 | B |
| ATOM | 939 | O | ILE | 136 | 33.149 | 11.258 | 47.255 | 1.00 | 18.78 | B |
| ATOM | 940 | N | PRO | 137 | 35.377 | 11.340 | 47.499 | 1.00 | 18.18 | B |
| ATOM | 941 | CD | PRO | 137 | 36.695 | 11.158 | 48.127 | 1.00 | 15.47 | B |
| ATOM | 942 | CA | PRO | 137 | 35.501 | 12.008 | 46.198 | 1.00 | 17.79 | B |
| ATOM | 943 | CB | PRO | 137 | 36.995 | 12.321 | 46.105 | 1.00 | 15.58 | B |
| ATOM | 944 | CG | PRO | 137 | 37.618 | 11.255 | 46.946 | 1.00 | 16.71 | B |
| ATOM | 945 | C | PRO | 137 | 35.010 | 11.135 | 45.040 | 1.00 | 20.22 | B |
| ATOM | 946 | O | PRO | 137 | 34.434 | 11.625 | 44.080 | 1.00 | 21.41 | B |
| ATOM | 947 | N | ARG | 138 | 35.234 | 9.829 | 45.135 | 1.00 | 22.72 | B |
| ATOM | 948 | CA | ARG | 138 | 34.789 | 8.927 | 44.075 | 1.00 | 22.41 | B |
| ATOM | 949 | CB | ARG | 138 | 35.378 | 7.534 | 44.270 | 1.00 | 21.69 | B |
| ATOM | 950 | CG | ARG | 138 | 36.860 | 7.433 | 43.951 | 1.00 | 20.35 | B |
| ATOM | 951 | CD | ARG | 138 | 37.395 | 6.072 | 44.347 | 1.00 | 17.89 | B |
| ATOM | 952 | NE | ARG | 138 | 38.847 | 6.020 | 44.275 | 1.00 | 17.83 | B |
| ATOM | 953 | CZ | ARG | 138 | 39.529 | 5.905 | 43.142 | 1.00 | 18.07 | B |
| ATOM | 954 | NH1 | ARG | 138 | 38.886 | 5.818 | 41.987 | 1.00 | 19.38 | B |
| ATOM | 955 | NH2 | ARG | 138 | 40.854 | 5.906 | 43.156 | 1.00 | 18.54 | B |
| ATOM | 956 | C | ARG | 138 | 33.263 | 8.829 | 44.007 | 1.00 | 22.14 | B |
| ATOM | 957 | O | ARG | 138 | 32.689 | 8.890 | 42.942 | 1.00 | 23.68 | B |
| ATOM | 958 | N | THR | 139 | 32.615 | 8.678 | 45.154 | 1.00 | 22.12 | B |
| ATOM | 959 | CA | THR | 139 | 31.161 | 8.566 | 45.203 | 1.00 | 25.57 | B |
| ATOM | 960 | CB | THR | 139 | 30.675 | 8.360 | 46.662 | 1.00 | 25.67 | B |
| ATOM | 961 | OG1 | THR | 139 | 31.355 | 7.236 | 47.234 | 1.00 | 27.07 | B |
| ATOM | 962 | CG2 | THR | 139 | 29.174 | 8.100 | 46.700 | 1.00 | 27.35 | B |
| ATOM | 963 | C | THR | 139 | 30.463 | 9.797 | 44.614 | 1.00 | 26.55 | B |
| ATOM | 964 | O | THR | 139 | 29.544 | 9.675 | 43.809 | 1.00 | 26.69 | B |
| ATOM | 965 | N | LEU | 140 | 30.910 | 10.982 | 45.017 | 1.00 | 27.11 | B |
| ATOM | 966 | CA | LEU | 140 | 30.314 | 12.213 | 44.523 | 1.00 | 26.17 | B |
| ATOM | 967 | CB | LEU | 140 | 30.949 | 13.424 | 45.209 | 1.00 | 26.20 | B |
| ATOM | 968 | CG | LEU | 140 | 30.599 | 13.605 | 46.690 | 1.00 | 26.65 | B |
| ATOM | 969 | CD1 | LEU | 140 | 31.435 | 14.723 | 47.280 | 1.00 | 25.28 | B |
| ATOM | 970 | CD2 | LEU | 140 | 29.114 | 13.896 | 46.849 | 1.00 | 24.93 | B |
| ATOM | 971 | C | LEU | 140 | 30.473 | 12.320 | 43.018 | 1.00 | 25.73 | B |
| ATOM | 972 | O | LEU | 140 | 29.556 | 12.725 | 42.333 | 1.00 | 25.93 | B |
| ATOM | 973 | N | HIS | 141 | 31.641 | 11.941 | 42.514 | 1.00 | 25.67 | B |
| ATOM | 974 | CA | HIS | 141 | 31.907 | 12.001 | 41.081 | 1.00 | 26.55 | B |
| ATOM | 975 | CB | HIS | 141 | 33.394 | 11.743 | 40.813 | 1.00 | 25.96 | B |
| ATOM | 976 | CG | HIS | 141 | 33.770 | 11.804 | 39.364 | 1.00 | 26.57 | B |
| ATOM | 977 | CD2 | HIS | 141 | 33.823 | 10.841 | 38.415 | 1.00 | 28.59 | B |
| ATOM | 978 | ND1 | HIS | 141 | 34.138 | 12.974 | 38.739 | 1.00 | 29.67 | B |
| ATOM | 979 | CE1 | HIS | 141 | 34.405 | 12.731 | 37.467 | 1.00 | 29.67 | B |
| ATOM | 980 | NE2 | HIS | 141 | 34.221 | 11.443 | 37.245 | 1.00 | 28.28 | B |
| ATOM | 981 | C | HIS | 141 | 31.072 | 10.973 | 40.322 | 1.00 | 26.86 | B |
| ATOM | 982 | O | HIS | 141 | 30.679 | 11.199 | 39.181 | 1.00 | 28.03 | B |
| ATOM | 983 | N | GLN | 142 | 30.802 | 9.844 | 40.965 | 1.00 | 24.80 | B |
| ATOM | 984 | CA | GLN | 142 | 30.045 | 8.780 | 40.326 | 1.00 | 25.14 | B |
| ATOM | 985 | CB | GLN | 142 | 30.353 | 7.436 | 40.994 | 1.00 | 27.48 | B |
| ATOM | 986 | CG | GLN | 142 | 31.680 | 6.834 | 40.563 | 1.00 | 30.52 | B |
| ATOM | 987 | CD | GLN | 142 | 31.684 | 6.417 | 39.102 | 1.00 | 34.29 | B |
| ATOM | 988 | OE1 | GLN | 142 | 30.990 | 5.475 | 38.711 | 1.00 | 34.96 | B |
| ATOM | 989 | NE2 | GLN | 142 | 32.468 | 7.116 | 38.287 | 1.00 | 35.49 | B |
| ATOM | 990 | C | GLN | 142 | 28.550 | 9.017 | 40.317 | 1.00 | 22.70 | B |
| ATOM | 991 | O | GLN | 142 | 27.856 | 8.528 | 39.440 | 1.00 | 21.46 | B |
| ATOM | 992 | N | ILE | 143 | 28.058 | 9.766 | 41.297 | 1.00 | 21.92 | B |
| ATOM | 993 | CA | ILE | 143 | 26.634 | 10.062 | 41.365 | 1.00 | 22.81 | B |
| ATOM | 994 | CB | ILE | 143 | 26.304 | 10.888 | 42.620 | 1.00 | 22.20 | B |
| ATOM | 995 | CG2 | ILE | 143 | 24.880 | 11.423 | 42.533 | 1.00 | 22.62 | B |
| ATOM | 996 | CG1 | ILE | 143 | 26.476 | 10.024 | 43.872 | 1.00 | 21.94 | B |
| ATOM | 997 | CD1 | ILE | 143 | 26.390 | 10.793 | 45.177 | 1.00 | 20.22 | B |
| ATOM | 998 | C | ILE | 143 | 26.187 | 10.824 | 40.114 | 1.00 | 24.31 | B |
| ATOM | 999 | O | ILE | 143 | 25.156 | 10.525 | 39.544 | 1.00 | 24.61 | B |
| ATOM | 1000 | N | PHE | 144 | 26.987 | 11.803 | 39.693 | 1.00 | 26.83 | B |
| ATOM | 1001 | CA | PHE | 144 | 26.672 | 12.611 | 38.511 | 1.00 | 28.06 | B |
| ATOM | 1002 | CB | PHE | 144 | 27.580 | 13.857 | 38.439 | 1.00 | 26.87 | B |
| ATOM | 1003 | CG | PHE | 144 | 27.330 | 14.861 | 39.536 | 1.00 | 27.89 | B |
| ATOM | 1004 | CD1 | PHE | 144 | 26.169 | 15.630 | 39.545 | 1.00 | 29.48 | B |
| ATOM | 1005 | CD2 | PHE | 144 | 28.230 | 15.002 | 40.592 | 1.00 | 28.77 | B |
| ATOM | 1006 | CE1 | PHE | 144 | 25.901 | 16.518 | 40.592 | 1.00 | 28.27 | B |
| ATOM | 1007 | CE2 | PHE | 144 | 27.974 | 15.890 | 41.647 | 1.00 | 28.13 | B |
| ATOM | 1008 | CZ | PHE | 144 | 26.805 | 16.646 | 41.646 | 1.00 | 30.04 | B |
| ATOM | 1009 | C | PHE | 144 | 26.818 | 11.778 | 37.238 | 1.00 | 28.29 | B |
| ATOM | 1010 | O | PHE | 144 | 26.140 | 12.025 | 36.253 | 1.00 | 28.71 | B |
| ATOM | 1011 | N | GLU | 145 | 27.703 | 10.786 | 37.273 | 1.00 | 29.40 | B |
| ATOM | 1012 | CA | GLU | 145 | 27.915 | 9.909 | 36.122 | 1.00 | 31.01 | B |
| ATOM | 1013 | CB | GLU | 145 | 29.216 | 9.129 | 36.297 | 1.00 | 32.65 | B |
| ATOM | 1014 | CG | GLU | 145 | 30.467 | 9.938 | 36.056 | 1.00 | 38.99 | B |
| ATOM | 1015 | CD | GLU | 145 | 30.706 | 10.197 | 34.578 | 1.00 | 43.44 | B |
| ATOM | 1016 | OE1 | GLU | 145 | 31.623 | 10.987 | 34.246 | 1.00 | 45.83 | B |
| ATOM | 1017 | OE2 | GLU | 145 | 29.977 | 9.603 | 33.752 | 1.00 | 45.50 | B |
| ATOM | 1018 | C | GLU | 145 | 26.753 | 8.926 | 35.940 | 1.00 | 31.44 | B |
| ATOM | 1019 | O | GLU | 145 | 26.237 | 8.754 | 34.841 | 1.00 | 30.51 | B |
| ATOM | 1020 | N | LYS | 146 | 26.348 | 8.290 | 37.033 | 1.00 | 31.75 | B |
| ATOM | 1021 | CA | LYS | 146 | 25.269 | 7.310 | 37.012 | 1.00 | 33.61 | B |
| ATOM | 1022 | CB | LYS | 146 | 25.172 | 6.629 | 38.381 | 1.00 | 34.03 | B |
| ATOM | 1023 | CG | LYS | 146 | 26.350 | 5.717 | 38.695 | 1.00 | 38.09 | B |
| ATOM | 1024 | CD | LYS | 146 | 26.243 | 5.107 | 40.086 | 1.00 | 40.00 | B |
| ATOM | 1025 | CE | LYS | 146 | 27.228 | 3.958 | 40.263 | 1.00 | 43.91 | B |
| ATOM | 1026 | NZ | LYS | 146 | 26.919 | 2.818 | 39.352 | 1.00 | 43.76 | B |
| ATOM | 1027 | C | LYS | 146 | 23.908 | 7.882 | 36.624 | 1.00 | 33.97 | B |
| ATOM | 1028 | O | LYS | 146 | 23.171 | 7.276 | 35.840 | 1.00 | 33.52 | B |
| ATOM | 1029 | N | LEU | 147 | 23.577 | 9.046 | 37.176 | 1.00 | 33.52 | B |
| ATOM | 1030 | CA | LEU | 147 | 22.302 | 9.689 | 36.892 | 1.00 | 32.92 | B |
| ATOM | 1031 | CB | LEU | 147 | 21.746 | 10.320 | 38.175 | 1.00 | 31.38 | B |
| ATOM | 1032 | CG | LEU | 147 | 21.336 | 9.359 | 39.302 | 1.00 | 32.23 | B |
| ATOM | 1033 | CD1 | LEU | 147 | 21.060 | 10.138 | 40.585 | 1.00 | 31.01 | B |
| ATOM | 1034 | CD2 | LEU | 147 | 20.096 | 8.569 | 38.883 | 1.00 | 32.23 | B |
| ATOM | 1035 | C | LEU | 147 | 22.418 | 10.749 | 35.794 | 1.00 | 32.85 | B |
| ATOM | 1036 | O | LEU | 147 | 21.562 | 11.609 | 35.669 | 1.00 | 33.29 | B |
| ATOM | 1037 | N | THR | 148 | 23.475 | 10.666 | 34.992 | 1.00 | 33.48 | B |
| ATOM | 1038 | CA | THR | 148 | 23.701 | 11.636 | 33.921 | 1.00 | 35.96 | B |
| ATOM | 1039 | CB | THR | 148 | 24.900 | 11.236 | 33.036 | 1.00 | 36.22 | B |
| ATOM | 1040 | OG1 | THR | 148 | 25.074 | 12.218 | 32.008 | 1.00 | 37.20 | B |
| ATOM | 1041 | CG2 | THR | 148 | 24.664 | 9.871 | 32.381 | 1.00 | 38.66 | B |
| ATOM | 1042 | C | THR | 148 | 22.484 | 11.879 | 33.014 | 1.00 | 36.52 | B |
| ATOM | 1043 | O | THR | 148 | 22.123 | 13.021 | 32.772 | 1.00 | 35.06 | B |
| ATOM | 1044 | N | ASP | 149 | 21.868 | 10.806 | 32.514 | 1.00 | 35.79 | B |
| ATOM | 1045 | CA | ASP | 149 | 20.690 | 10.923 | 31.648 | 1.00 | 35.29 | B |
| ATOM | 1046 | CB | ASP | 149 | 21.101 | 11.265 | 30.206 | 1.00 | 36.06 | B |
| ATOM | 1047 | CG | ASP | 149 | 22.065 | 10.249 | 29.607 | 1.00 | 37.80 | B |
| ATOM | 1048 | OD1 | ASP | 149 | 22.292 | 9.196 | 30.243 | 1.00 | 40.41 | B |
| ATOM | 1049 | OD2 | ASP | 149 | 22.590 | 10.500 | 28.496 | 1.00 | 36.11 | B |
| ATOM | 1050 | C | ASP | 149 | 19.821 | 9.657 | 31.646 | 1.00 | 34.60 | B |
| ATOM | 1051 | O | ASP | 149 | 19.397 | 9.184 | 30.592 | 1.00 | 31.15 | B |
| ATOM | 1052 | N | ASN | 150 | 19.554 | 9.122 | 32.834 | 1.00 | 34.29 | B |
| ATOM | 1053 | CA | ASN | 150 | 18.732 | 7.923 | 32.948 | 1.00 | 35.52 | B |
| ATOM | 1054 | CB | ASN | 150 | 19.227 | 7.041 | 34.102 | 1.00 | 32.56 | B |
| ATOM | 1055 | CG | ASN | 150 | 19.031 | 7.690 | 35.452 | 1.00 | 32.34 | B |
| ATOM | 1056 | OD1 | ASN | 150 | 19.134 | 8.903 | 35.579 | 1.00 | 29.46 | B |
| ATOM | 1057 | ND2 | ASN | 150 | 18.760 | 6.877 | 36.475 | 1.00 | 31.14 | B |
| ATOM | 1058 | C | ASN | 150 | 17.265 | 8.292 | 33.154 | 1.00 | 36.96 | B |
| ATOM | 1059 | O | ASN | 150 | 16.436 | 7.431 | 33.447 | 1.00 | 37.74 | B |
| ATOM | 1060 | N | GLY | 151 | 16.953 | 9.578 | 32.996 | 1.00 | 37.37 | B |
| ATOM | 1061 | CA | GLY | 151 | 15.585 | 10.044 | 33.153 | 1.00 | 37.75 | B |
| ATOM | 1062 | C | GLY | 151 | 15.195 | 10.351 | 34.585 | 1.00 | 39.12 | B |
| ATOM | 1063 | O | GLY | 151 | 14.013 | 10.490 | 34.903 | 1.00 | 39.41 | B |
| ATOM | 1064 | N | THR | 152 | 16.190 | 10.455 | 35.455 | 1.00 | 40.74 | B |
| ATOM | 1065 | CA | THR | 152 | 15.950 | 10.748 | 36.860 | 1.00 | 42.40 | B |
| ATOM | 1066 | CB | THR | 152 | 16.587 | 9.674 | 37.772 | 1.00 | 42.88 | B |
| ATOM | 1067 | OG1 | THR | 152 | 16.143 | 8.375 | 37.365 | 1.00 | 46.42 | B |
| ATOM | 1068 | CG2 | THR | 152 | 16.182 | 9.891 | 39.221 | 1.00 | 43.02 | B |
| ATOM | 1069 | C | THR | 152 | 16.537 | 12.108 | 37.216 | 1.00 | 42.92 | B |
| ATOM | 1070 | O | THR | 152 | 17.753 | 12.303 | 37.176 | 1.00 | 45.15 | B |
| ATOM | 1071 | N | GLU | 153 | 15.657 | 13.050 | 37.539 | 1.00 | 41.16 | B |
| ATOM | 1072 | CA | GLU | 153 | 16.083 | 14.390 | 37.910 | 1.00 | 39.15 | B |
| ATOM | 1073 | CB | GLU | 153 | 14.902 | 15.350 | 37.865 | 1.00 | 41.46 | B |
| ATOM | 1074 | CG | GLU | 153 | 15.290 | 16.742 | 37.456 | 1.00 | 46.88 | B |
| ATOM | 1075 | CD | GLU | 153 | 15.645 | 16.826 | 35.983 | 1.00 | 50.26 | B |
| ATOM | 1076 | OE1 | GLU | 153 | 16.309 | 17.808 | 35.591 | 1.00 | 54.28 | B |
| ATOM | 1077 | OE2 | GLU | 153 | 15.256 | 15.920 | 35.216 | 1.00 | 50.49 | B |
| ATOM | 1078 | C | GLU | 153 | 16.601 | 14.273 | 39.336 | 1.00 | 35.77 | B |
| ATOM | 1079 | O | GLU | 153 | 16.024 | 13.550 | 40.143 | 1.00 | 34.39 | B |
| ATOM | 1080 | N | PHE | 154 | 17.676 | 14.986 | 39.649 | 1.00 | 32.19 | B |
| ATOM | 1081 | CA | PHE | 154 | 18.247 | 14.903 | 40.985 | 1.00 | 29.64 | B |
| ATOM | 1082 | CB | PHE | 154 | 19.221 | 13.731 | 41.036 | 1.00 | 26.07 | B |
| ATOM | 1083 | CG | PHE | 154 | 20.478 | 13.959 | 40.244 | 1.00 | 22.24 | B |
| ATOM | 1084 | CD1 | PHE | 154 | 21.634 | 14.413 | 40.870 | 1.00 | 19.12 | B |
| ATOM | 1085 | CD2 | PHE | 154 | 20.502 | 13.725 | 38.873 | 1.00 | 19.79 | B |
| ATOM | 1086 | CE1 | PHE | 154 | 22.804 | 14.627 | 40.140 | 1.00 | 20.17 | B |
| ATOM | 1087 | CE2 | PHE | 154 | 21.665 | 13.938 | 38.132 | 1.00 | 19.68 | B |
| ATOM | 1088 | CZ | PHE | 154 | 22.819 | 14.388 | 38.768 | 1.00 | 18.22 | B |
| ATOM | 1089 | C | PHE | 154 | 18.983 | 16.153 | 41.462 | 1.00 | 28.59 | B |
| ATOM | 1090 | O | PHE | 154 | 19.343 | 17.025 | 40.687 | 1.00 | 28.03 | B |
| ATOM | 1091 | N | SER | 155 | 19.219 | 16.194 | 42.765 | 1.00 | 28.62 | B |
| ATOM | 1092 | CA | SER | 155 | 19.940 | 17.286 | 43.398 | 1.00 | 29.65 | B |
| ATOM | 1093 | CB | SER | 155 | 18.958 | 18.297 | 44.007 | 1.00 | 29.30 | B |
| ATOM | 1094 | OG | SER | 155 | 18.373 | 17.825 | 45.210 | 1.00 | 30.25 | B |
| ATOM | 1095 | C | SER | 155 | 20.812 | 16.670 | 44.495 | 1.00 | 29.32 | B |
| ATOM | 1096 | O | SER | 155 | 20.364 | 15.799 | 45.236 | 1.00 | 28.78 | B |
| ATOM | 1097 | N | VAL | 156 | 22.057 | 17.117 | 44.601 | 1.00 | 28.25 | B |
| ATOM | 1098 | CA | VAL | 156 | 22.945 | 16.571 | 45.622 | 1.00 | 27.65 | B |
| ATOM | 1099 | CB | VAL | 156 | 24.266 | 16.059 | 45.002 | 1.00 | 27.82 | B |
| ATOM | 1100 | CG1 | VAL | 156 | 25.067 | 15.296 | 46.051 | 1.00 | 26.25 | B |
| ATOM | 1101 | CG2 | VAL | 156 | 23.970 | 15.178 | 43.793 | 1.00 | 26.92 | B |
| ATOM | 1102 | C | VAL | 156 | 23.293 | 17.600 | 46.697 | 1.00 | 28.00 | B |
| ATOM | 1103 | O | VAL | 156 | 23.691 | 18.705 | 46.386 | 1.00 | 27.61 | B |
| ATOM | 1104 | N | LYS | 157 | 23.135 | 17.210 | 47.961 | 1.00 | 28.26 | B |
| ATOM | 1105 | CA | LYS | 157 | 23.455 | 18.066 | 49.107 | 1.00 | 29.25 | B |
| ATOM | 1106 | CB | LYS | 157 | 22.188 | 18.423 | 49.897 | 1.00 | 30.98 | B |
| ATOM | 1107 | CG | LYS | 157 | 21.322 | 19.485 | 49.261 | 1.00 | 34.09 | B |
| ATOM | 1108 | CD | LYS | 157 | 20.065 | 19.741 | 50.080 | 1.00 | 37.95 | B |
| ATOM | 1109 | CE | LYS | 157 | 19.399 | 21.060 | 49.665 | 1.00 | 41.02 | B |
| ATOM | 1110 | NZ | LYS | 157 | 20.186 | 22.277 | 50.077 | 1.00 | 41.43 | B |
| ATOM | 1111 | C | LYS | 157 | 24.426 | 17.349 | 50.047 | 1.00 | 28.34 | B |
| ATOM | 1112 | O | LYS | 157 | 24.195 | 16.217 | 50.413 | 1.00 | 28.14 | B |
| ATOM | 1113 | N | VAL | 158 | 25.510 | 18.016 | 50.433 | 1.00 | 27.07 | B |
| ATOM | 1114 | CA | VAL | 158 | 26.480 | 17.412 | 51.342 | 1.00 | 27.48 | B |
| ATOM | 1115 | CB | VAL | 158 | 27.883 | 17.280 | 50.694 | 1.00 | 26.91 | B |
| ATOM | 1116 | CG1 | VAL | 158 | 27.811 | 16.356 | 49.489 | 1.00 | 27.77 | B |
| ATOM | 1117 | CG2 | VAL | 158 | 28.415 | 18.648 | 50.301 | 1.00 | 27.25 | B |
| ATOM | 1118 | C | VAL | 158 | 26.629 | 18.183 | 52.651 | 1.00 | 28.66 | B |
| ATOM | 1119 | O | VAL | 158 | 26.444 | 19.393 | 52.705 | 1.00 | 27.69 | B |
| ATOM | 1120 | N | SER | 159 | 26.973 | 17.460 | 53.708 | 1.00 | 28.98 | B |
| ATOM | 1121 | CA | SER | 159 | 27.155 | 18.058 | 55.013 | 1.00 | 30.95 | B |
| ATOM | 1122 | CB | SER | 159 | 25.869 | 17.953 | 55.823 | 1.00 | 32.26 | B |
| ATOM | 1123 | OG | SER | 159 | 24.817 | 18.602 | 55.132 | 1.00 | 38.42 | B |
| ATOM | 1124 | C | SER | 159 | 28.289 | 17.362 | 55.736 | 1.00 | 30.96 | B |
| ATOM | 1125 | O | SER | 159 | 28.388 | 16.146 | 55.722 | 1.00 | 34.27 | B |
| ATOM | 1126 | N | LEU | 160 | 29.158 | 18.143 | 56.357 | 1.00 | 29.31 | B |
| ATOM | 1127 | CA | LEU | 160 | 30.280 | 17.577 | 57.064 | 1.00 | 27.33 | B |
| ATOM | 1128 | CB | LEU | 160 | 31.582 | 18.130 | 56.499 | 1.00 | 27.18 | B |
| ATOM | 1129 | CG | LEU | 160 | 32.856 | 17.456 | 56.991 | 1.00 | 28.13 | B |
| ATOM | 1130 | CD1 | LEU | 160 | 32.751 | 15.954 | 56.790 | 1.00 | 29.56 | B |
| ATOM | 1131 | CD2 | LEU | 160 | 34.044 | 18.019 | 56.237 | 1.00 | 28.17 | B |
| ATOM | 1132 | C | LEU | 160 | 30.167 | 17.884 | 58.552 | 1.00 | 28.09 | B |
| ATOM | 1133 | O | LEU | 160 | 30.607 | 18.943 | 59.026 | 1.00 | 26.39 | B |
| ATOM | 1134 | N | LEU | 161 | 29.558 | 16.949 | 59.276 | 1.00 | 25.48 | B |
| ATOM | 1135 | CA | LEU | 161 | 29.371 | 17.075 | 60.710 | 1.00 | 23.19 | B |
| ATOM | 1136 | CB | LEU | 161 | 27.982 | 16.567 | 61.101 | 1.00 | 21.33 | B |
| ATOM | 1137 | CG | LEU | 161 | 27.694 | 16.395 | 62.594 | 1.00 | 19.50 | B |
| ATOM | 1138 | CD1 | LEU | 161 | 27.772 | 17.736 | 63.288 | 1.00 | 19.94 | B |
| ATOM | 1139 | CD2 | LEU | 161 | 26.314 | 15.775 | 62.782 | 1.00 | 17.88 | B |
| ATOM | 1140 | C | LEU | 161 | 30.452 | 16.264 | 61.415 | 1.00 | 23.39 | B |
| ATOM | 1141 | O | LEU | 161 | 30.641 | 15.094 | 61.129 | 1.00 | 25.56 | B |
| ATOM | 1142 | N | GLU | 162 | 31.165 | 16.899 | 62.336 | 1.00 | 22.32 | B |
| ATOM | 1143 | CA | GLU | 162 | 32.232 | 16.237 | 63.065 | 1.00 | 19.98 | B |
| ATOM | 1144 | CB | GLU | 162 | 33.574 | 16.839 | 62.650 | 1.00 | 17.28 | B |
| ATOM | 1145 | CG | GLU | 162 | 33.762 | 16.859 | 61.137 | 1.00 | 15.11 | B |
| ATOM | 1146 | CD | GLU | 162 | 35.212 | 16.937 | 60.737 | 1.00 | 15.23 | B |
| ATOM | 1147 | OE1 | GLU | 162 | 36.063 | 17.134 | 61.621 | 1.00 | 15.82 | B |
| ATOM | 1148 | OE2 | GLU | 162 | 35.513 | 16.813 | 59.539 | 1.00 | 17.71 | B |
| ATOM | 1149 | C | GLU | 162 | 32.031 | 16.344 | 64.573 | 1.00 | 19.72 | B |
| ATOM | 1150 | O | GLU | 162 | 31.468 | 17.299 | 65.059 | 1.00 | 20.94 | B |
| ATOM | 1151 | N | ILE | 163 | 32.503 | 15.348 | 65.312 | 1.00 | 18.63 | B |
| ATOM | 1152 | CA | ILE | 163 | 32.346 | 15.350 | 66.756 | 1.00 | 18.63 | B |
| ATOM | 1153 | CB | ILE | 163 | 31.544 | 14.120 | 67.223 | 1.00 | 19.02 | B |
| ATOM | 1154 | CG2 | ILE | 163 | 31.324 | 14.178 | 68.742 | 1.00 | 16.34 | B |
| ATOM | 1155 | CG1 | ILE | 163 | 30.210 | 14.072 | 66.466 | 1.00 | 20.01 | B |
| ATOM | 1156 | CD1 | ILE | 163 | 29.479 | 12.746 | 66.563 | 1.00 | 22.19 | B |
| ATOM | 1157 | C | ILE | 163 | 33.694 | 15.353 | 67.467 | 1.00 | 20.32 | B |
| ATOM | 1158 | O | ILE | 163 | 34.616 | 14.672 | 67.050 | 1.00 | 21.59 | B |
| ATOM | 1159 | N | TYR | 164 | 33.799 | 16.131 | 68.542 | 1.00 | 20.27 | B |
| ATOM | 1160 | CA | TYR | 164 | 35.031 | 16.206 | 69.312 | 1.00 | 19.81 | B |
| ATOM | 1161 | CB | TYR | 164 | 35.964 | 17.271 | 68.709 | 1.00 | 20.16 | B |
| ATOM | 1162 | CG | TYR | 164 | 37.269 | 17.434 | 69.451 | 1.00 | 17.18 | B |
| ATOM | 1163 | CD1 | TYR | 164 | 37.334 | 18.191 | 70.622 | 1.00 | 16.03 | B |
| ATOM | 1164 | CE1 | TYR | 164 | 38.506 | 18.253 | 71.372 | 1.00 | 16.71 | B |
| ATOM | 1165 | CD2 | TYR | 164 | 38.416 | 16.756 | 69.042 | 1.00 | 18.67 | B |
| ATOM | 1166 | CE2 | TYR | 164 | 39.594 | 16.812 | 69.789 | 1.00 | 16.74 | B |
| ATOM | 1167 | CZ | TYR | 164 | 39.627 | 17.557 | 70.954 | 1.00 | 14.83 | B |
| ATOM | 1168 | OH | TYR | 164 | 40.758 | 17.569 | 71.726 | 1.00 | 14.97 | B |
| ATOM | 1169 | C | TYR | 164 | 34.685 | 16.520 | 70.761 | 1.00 | 21.32 | B |
| ATOM | 1170 | O | TYR | 164 | 33.971 | 17.468 | 71.044 | 1.00 | 22.71 | B |
| ATOM | 1171 | N | ASN | 165 | 35.185 | 15.694 | 71.672 | 1.00 | 22.32 | B |
| ATOM | 1172 | CA | ASN | 165 | 34.926 | 15.860 | 73.092 | 1.00 | 23.78 | B |
| ATOM | 1173 | CB | ASN | 165 | 35.722 | 17.043 | 73.636 | 1.00 | 27.16 | B |
| ATOM | 1174 | CG | ASN | 165 | 35.729 | 17.090 | 75.149 | 1.00 | 31.99 | B |
| ATOM | 1175 | OD1 | ASN | 165 | 36.159 | 16.150 | 75.801 | 1.00 | 37.27 | B |
| ATOM | 1176 | ND2 | ASN | 165 | 35.249 | 18.190 | 75.714 | 1.00 | 32.43 | B |
| ATOM | 1177 | C | ASN | 165 | 33.431 | 16.088 | 73.313 | 1.00 | 24.23 | B |
| ATOM | 1178 | O | ASN | 165 | 33.034 | 16.915 | 74.130 | 1.00 | 25.34 | B |
| ATOM | 1179 | N | GLU | 166 | 32.615 | 15.340 | 72.572 | 1.00 | 22.37 | B |
| ATOM | 1180 | CA | GLU | 166 | 31.154 | 15.421 | 72.641 | 1.00 | 22.51 | B |
| ATOM | 1181 | CB | GLU | 166 | 30.638 | 15.047 | 74.044 | 1.00 | 19.36 | B |
| ATOM | 1182 | CG | GLU | 166 | 30.620 | 13.540 | 74.319 | 1.00 | 20.22 | B |
| ATOM | 1183 | CD | GLU | 166 | 29.915 | 12.746 | 73.222 | 1.00 | 20.01 | B |
| ATOM | 1184 | OE1 | GLU | 166 | 28.668 | 12.648 | 73.240 | 1.00 | 19.99 | B |
| ATOM | 1185 | OE2 | GLU | 166 | 30.618 | 12.228 | 72.330 | 1.00 | 16.45 | B |
| ATOM | 1186 | C | GLU | 166 | 30.570 | 16.770 | 72.223 | 1.00 | 22.98 | B |
| ATOM | 1187 | O | GLU | 166 | 29.553 | 17.189 | 72.725 | 1.00 | 22.40 | B |
| ATOM | 1188 | N | GLU | 167 | 31.229 | 17.443 | 71.288 | 1.00 | 25.41 | B |
| ATOM | 1189 | CA | GLU | 167 | 30.739 | 18.721 | 70.793 | 1.00 | 27.30 | B |
| ATOM | 1190 | CB | GLU | 167 | 31.679 | 19.858 | 71.191 | 1.00 | 29.98 | B |
| ATOM | 1191 | CG | GLU | 167 | 31.567 | 20.295 | 72.648 | 1.00 | 34.85 | B |
| ATOM | 1192 | CD | GLU | 167 | 32.384 | 21.553 | 72.941 | 1.00 | 39.75 | B |
| ATOM | 1193 | OE1 | GLU | 167 | 33.635 | 21.487 | 72.865 | 1.00 | 39.56 | B |
| ATOM | 1194 | OE2 | GLU | 167 | 31.771 | 22.608 | 73.237 | 1.00 | 41.26 | B |
| ATOM | 1195 | C | GLU | 167 | 30.637 | 18.626 | 69.278 | 1.00 | 28.54 | B |
| ATOM | 1196 | O | GLU | 167 | 31.495 | 18.046 | 68.633 | 1.00 | 29.56 | B |
| ATOM | 1197 | N | LEU | 168 | 29.574 | 19.190 | 68.719 | 1.00 | 28.34 | B |
| ATOM | 1198 | CA | LEU | 168 | 29.367 | 19.138 | 67.280 | 1.00 | 28.28 | B |
| ATOM | 1199 | CB | LEU | 168 | 27.865 | 19.078 | 66.955 | 1.00 | 30.49 | B |
| ATOM | 1200 | CG | LEU | 168 | 27.009 | 17.925 | 67.512 | 1.00 | 30.82 | B |
| ATOM | 1201 | CD1 | LEU | 168 | 27.623 | 16.583 | 67.142 | 1.00 | 31.07 | B |
| ATOM | 1202 | CD2 | LEU | 168 | 26.892 | 18.044 | 69.009 | 1.00 | 33.15 | B |
| ATOM | 1203 | C | LEU | 168 | 29.997 | 20.322 | 66.563 | 1.00 | 26.93 | B |
| ATOM | 1204 | O | LEU | 168 | 29.972 | 21.442 | 67.064 | 1.00 | 28.48 | B |
| ATOM | 1205 | N | PHE | 169 | 30.562 | 20.069 | 65.386 | 1.00 | 24.01 | B |
| ATOM | 1206 | CA | PHE | 169 | 31.191 | 21.112 | 64.584 | 1.00 | 22.58 | B |
| ATOM | 1207 | CB | PHE | 169 | 32.723 | 21.073 | 64.727 | 1.00 | 22.71 | B |
| ATOM | 1208 | CG | PHE | 169 | 33.213 | 21.377 | 66.118 | 1.00 | 21.76 | B |
| ATOM | 1209 | CD1 | PHE | 169 | 33.451 | 20.354 | 67.027 | 1.00 | 21.14 | B |
| ATOM | 1210 | CD2 | PHE | 169 | 33.393 | 22.699 | 66.534 | 1.00 | 22.60 | B |
| ATOM | 1211 | CE1 | PHE | 169 | 33.861 | 20.628 | 68.323 | 1.00 | 22.05 | B |
| ATOM | 1212 | CE2 | PHE | 169 | 33.802 | 22.989 | 67.830 | 1.00 | 21.62 | B |
| ATOM | 1213 | CZ | PHE | 169 | 34.037 | 21.952 | 68.729 | 1.00 | 24.67 | B |
| ATOM | 1214 | C | PHE | 169 | 30.824 | 20.950 | 63.111 | 1.00 | 23.10 | B |
| ATOM | 1215 | O | PHE | 169 | 30.612 | 19.836 | 62.634 | 1.00 | 20.06 | B |
| ATOM | 1216 | N | ASP | 170 | 30.739 | 22.079 | 62.406 | 1.00 | 22.96 | B |
| ATOM | 1217 | CA | ASP | 170 | 30.416 | 22.100 | 60.978 | 1.00 | 22.20 | B |
| ATOM | 1218 | CB | ASP | 170 | 29.344 | 23.148 | 60.679 | 1.00 | 20.54 | B |
| ATOM | 1219 | CG | ASP | 170 | 28.799 | 23.048 | 59.257 | 1.00 | 21.66 | B |
| ATOM | 1220 | OD1 | ASP | 170 | 29.554 | 22.671 | 58.337 | 1.00 | 18.77 | B |
| ATOM | 1221 | OD2 | ASP | 170 | 27.602 | 23.358 | 59.065 | 1.00 | 23.66 | B |
| ATOM | 1222 | C | ASP | 170 | 31.680 | 22.466 | 60.211 | 1.00 | 22.85 | B |
| ATOM | 1223 | O | ASP | 170 | 32.108 | 23.621 | 60.242 | 1.00 | 25.36 | B |
| ATOM | 1224 | N | LEU | 171 | 32.280 | 21.490 | 59.529 | 1.00 | 22.35 | B |
| ATOM | 1225 | CA | LEU | 171 | 33.494 | 21.729 | 58.764 | 1.00 | 22.58 | B |
| ATOM | 1226 | CB | LEU | 171 | 34.430 | 20.533 | 58.864 | 1.00 | 16.27 | B |
| ATOM | 1227 | CG | LEU | 171 | 35.235 | 20.424 | 60.169 | 1.00 | 16.39 | B |
| ATOM | 1228 | CD1 | LEU | 171 | 36.234 | 21.577 | 60.274 | 1.00 | 14.32 | B |
| ATOM | 1229 | CD2 | LEU | 171 | 34.304 | 20.421 | 61.351 | 1.00 | 12.71 | B |
| ATOM | 1230 | C | LEU | 171 | 33.257 | 22.082 | 57.300 | 1.00 | 26.58 | B |
| ATOM | 1231 | O | LEU | 171 | 34.167 | 21.976 | 56.479 | 1.00 | 26.75 | B |
| ATOM | 1232 | N | LEU | 172 | 32.038 | 22.510 | 56.978 | 1.00 | 29.45 | B |
| ATOM | 1233 | CA | LEU | 172 | 31.706 | 22.898 | 55.612 | 1.00 | 34.57 | B |
| ATOM | 1234 | CB | LEU | 172 | 30.742 | 21.892 | 54.975 | 1.00 | 33.36 | B |
| ATOM | 1235 | CG | LEU | 172 | 31.387 | 20.715 | 54.244 | 1.00 | 31.35 | B |
| ATOM | 1236 | CD1 | LEU | 172 | 30.316 | 19.992 | 53.459 | 1.00 | 32.85 | B |
| ATOM | 1237 | CD2 | LEU | 172 | 32.473 | 21.201 | 53.302 | 1.00 | 32.08 | B |
| ATOM | 1238 | C | LEU | 172 | 31.107 | 24.297 | 55.531 | 1.00 | 38.00 | B |
| ATOM | 1239 | O | LEU | 172 | 30.961 | 24.850 | 54.457 | 1.00 | 39.59 | B |
| ATOM | 1240 | N | ASN | 173 | 30.766 | 24.865 | 56.679 | 1.00 | 41.36 | B |
| ATOM | 1241 | CA | ASN | 173 | 30.201 | 26.205 | 56.714 | 1.00 | 45.99 | B |
| ATOM | 1242 | CB | ASN | 173 | 29.401 | 26.405 | 58.003 | 1.00 | 47.65 | B |
| ATOM | 1243 | CG | ASN | 173 | 28.670 | 27.735 | 58.038 | 1.00 | 50.77 | B |
| ATOM | 1244 | OD1 | ASN | 173 | 28.005 | 28.060 | 59.014 | 1.00 | 51.85 | B |
| ATOM | 1245 | ND2 | ASN | 173 | 28.792 | 28.508 | 56.964 | 1.00 | 51.20 | B |
| ATOM | 1246 | C | ASN | 173 | 31.346 | 27.214 | 56.643 | 1.00 | 48.84 | B |
| ATOM | 1247 | O | ASN | 173 | 32.070 | 27.403 | 57.606 | 1.00 | 48.46 | B |
| ATOM | 1248 | N | PRO | 174 | 31.521 | 27.872 | 55.484 | 1.00 | 52.47 | B |
| ATOM | 1249 | CD | PRO | 174 | 30.710 | 27.738 | 54.258 | 1.00 | 53.23 | B |
| ATOM | 1250 | CA | PRO | 174 | 32.587 | 28.862 | 55.289 | 1.00 | 55.00 | B |
| ATOM | 1251 | CB | PRO | 174 | 32.542 | 29.116 | 53.786 | 1.00 | 53.92 | B |
| ATOM | 1252 | CG | PRO | 174 | 31.089 | 28.983 | 53.482 | 1.00 | 52.93 | B |
| ATOM | 1253 | C | PRO | 174 | 32.396 | 30.141 | 56.095 | 1.00 | 58.07 | B |
| ATOM | 1254 | O | PRO | 174 | 33.329 | 30.921 | 56.263 | 1.00 | 58.84 | B |
| ATOM | 1255 | N | SER | 175 | 31.183 | 30.343 | 56.596 | 1.00 | 60.39 | B |
| ATOM | 1256 | CA | SER | 175 | 30.861 | 31.534 | 57.372 | 1.00 | 62.65 | B |
| ATOM | 1257 | CB | SER | 175 | 29.343 | 31.666 | 57.498 | 1.00 | 63.30 | B |
| ATOM | 1258 | OG | SER | 175 | 28.723 | 31.545 | 56.230 | 1.00 | 65.14 | B |
| ATOM | 1259 | C | SER | 175 | 31.500 | 31.535 | 58.759 | 1.00 | 63.89 | B |
| ATOM | 1260 | O | SER | 175 | 32.365 | 32.358 | 59.051 | 1.00 | 65.71 | B |
| ATOM | 1261 | N | SER | 176 | 31.066 | 30.608 | 59.608 | 1.00 | 64.41 | B |
| ATOM | 1262 | CA | SER | 176 | 31.581 | 30.506 | 60.969 | 1.00 | 64.51 | B |
| ATOM | 1263 | CB | SER | 176 | 30.597 | 29.725 | 61.844 | 1.00 | 64.33 | B |
| ATOM | 1264 | OG | SER | 176 | 30.446 | 28.396 | 61.378 | 1.00 | 64.08 | B |
| ATOM | 1265 | C | SER | 176 | 32.942 | 29.824 | 61.012 | 1.00 | 64.78 | B |
| ATOM | 1266 | O | SER | 176 | 33.474 | 29.418 | 59.984 | 1.00 | 64.25 | B |
| ATOM | 1267 | N | ASP | 177 | 33.500 | 29.704 | 62.213 | 1.00 | 65.17 | B |
| ATOM | 1268 | CA | ASP | 177 | 34.789 | 29.051 | 62.379 | 1.00 | 65.62 | B |
| ATOM | 1269 | CB | ASP | 177 | 35.782 | 29.964 | 63.106 | 1.00 | 66.73 | B |
| ATOM | 1270 | CG | ASP | 177 | 35.449 | 30.137 | 64.576 | 1.00 | 68.48 | B |
| ATOM | 1271 | OD1 | ASP | 177 | 36.388 | 30.344 | 65.377 | 1.00 | 67.76 | B |
| ATOM | 1272 | OD2 | ASP | 177 | 34.251 | 30.069 | 64.929 | 1.00 | 69.81 | B |
| ATOM | 1273 | C | ASP | 177 | 34.615 | 27.757 | 63.166 | 1.00 | 64.60 | B |
| ATOM | 1274 | O | ASP | 177 | 33.498 | 27.335 | 63.445 | 1.00 | 64.22 | B |
| ATOM | 1275 | N | VAL | 178 | 35.737 | 27.146 | 63.529 | 1.00 | 63.40 | B |
| ATOM | 1276 | CA | VAL | 178 | 35.735 | 25.890 | 64.264 | 1.00 | 62.69 | B |
| ATOM | 1277 | CB | VAL | 178 | 37.046 | 25.116 | 64.016 | 1.00 | 62.85 | B |
| ATOM | 1278 | CG1 | VAL | 178 | 37.190 | 24.809 | 62.536 | 1.00 | 61.71 | B |
| ATOM | 1279 | CG2 | VAL | 178 | 38.231 | 25.934 | 64.510 | 1.00 | 62.99 | B |
| ATOM | 1280 | C | VAL | 178 | 35.552 | 26.050 | 65.770 | 1.00 | 61.94 | B |
| ATOM | 1281 | O | VAL | 178 | 35.792 | 25.122 | 66.524 | 1.00 | 62.60 | B |
| ATOM | 1282 | N | SER | 179 | 35.124 | 27.227 | 66.208 | 1.00 | 61.07 | B |
| ATOM | 1283 | CA | SER | 179 | 34.922 | 27.447 | 67.632 | 1.00 | 59.46 | B |
| ATOM | 1284 | CB | SER | 179 | 35.629 | 28.731 | 68.080 | 1.00 | 59.42 | B |
| ATOM | 1285 | OG | SER | 179 | 35.030 | 29.877 | 67.507 | 1.00 | 59.13 | B |
| ATOM | 1286 | C | SER | 179 | 33.437 | 27.517 | 67.977 | 1.00 | 58.68 | B |
| ATOM | 1287 | O | SER | 179 | 33.067 | 27.489 | 69.144 | 1.00 | 59.17 | B |
| ATOM | 1288 | N | GLU | 180 | 32.591 | 27.605 | 66.955 | 1.00 | 56.65 | B |
| ATOM | 1289 | CA | GLU | 180 | 31.145 | 27.671 | 67.161 | 1.00 | 55.22 | B |
| ATOM | 1290 | CB | GLU | 180 | 30.507 | 28.607 | 66.129 | 1.00 | 56.66 | B |
| ATOM | 1291 | CG | GLU | 180 | 30.550 | 30.079 | 66.535 | 1.00 | 59.12 | B |
| ATOM | 1292 | CD | GLU | 180 | 30.230 | 31.032 | 65.392 | 1.00 | 60.03 | B |
| ATOM | 1293 | OE1 | GLU | 180 | 31.066 | 31.163 | 64.474 | 1.00 | 60.45 | B |
| ATOM | 1294 | OE2 | GLU | 180 | 29.143 | 31.650 | 65.411 | 1.00 | 61.47 | B |
| ATOM | 1295 | C | GLU | 180 | 30.498 | 26.293 | 67.080 | 1.00 | 52.95 | B |
| ATOM | 1296 | O | GLU | 180 | 30.207 | 25.803 | 66.004 | 1.00 | 52.86 | B |
| ATOM | 1297 | N | ARG | 181 | 30.285 | 25.679 | 68.239 | 1.00 | 51.12 | B |
| ATOM | 1298 | CA | ARG | 181 | 29.675 | 24.360 | 68.315 | 1.00 | 48.73 | B |
| ATOM | 1299 | CB | ARG | 181 | 29.835 | 23.793 | 69.727 | 1.00 | 51.62 | B |
| ATOM | 1300 | CG | ARG | 181 | 29.642 | 24.816 | 70.836 | 1.00 | 56.45 | B |
| ATOM | 1301 | CD | ARG | 181 | 28.829 | 24.256 | 72.007 | 1.00 | 61.65 | B |
| ATOM | 1302 | NE | ARG | 181 | 27.400 | 24.135 | 71.702 | 1.00 | 64.33 | B |
| ATOM | 1303 | CZ | ARG | 181 | 26.483 | 23.692 | 72.560 | 1.00 | 65.71 | B |
| ATOM | 1304 | NH1 | ARG | 181 | 26.834 | 23.324 | 73.786 | 1.00 | 66.05 | B |
| ATOM | 1305 | NH2 | ARG | 181 | 25.209 | 23.616 | 72.194 | 1.00 | 66.36 | B |
| ATOM | 1306 | C | ARG | 181 | 28.196 | 24.403 | 67.940 | 1.00 | 45.46 | B |
| ATOM | 1307 | O | ARG | 181 | 27.556 | 25.438 | 68.029 | 1.00 | 45.33 | B |
| ATOM | 1308 | N | LEU | 182 | 27.661 | 23.267 | 67.510 | 1.00 | 41.98 | B |
| ATOM | 1309 | CA | LEU | 182 | 26.258 | 23.193 | 67.133 | 1.00 | 38.04 | B |
| ATOM | 1310 | CB | LEU | 182 | 26.099 | 22.419 | 65.824 | 1.00 | 35.02 | B |
| ATOM | 1311 | CG | LEU | 182 | 26.990 | 22.896 | 64.677 | 1.00 | 33.00 | B |
| ATOM | 1312 | CD1 | LEU | 182 | 26.723 | 22.060 | 63.450 | 1.00 | 31.57 | B |
| ATOM | 1313 | CD2 | LEU | 182 | 26.733 | 24.372 | 64.393 | 1.00 | 32.49 | B |
| ATOM | 1314 | C | LEU | 182 | 25.456 | 22.524 | 68.236 | 1.00 | 38.00 | B |
| ATOM | 1315 | O | LEU | 182 | 26.017 | 21.845 | 69.096 | 1.00 | 37.75 | B |
| ATOM | 1316 | N | GLN | 183 | 24.140 | 22.723 | 68.206 | 1.00 | 37.43 | B |
| ATOM | 1317 | CA | GLN | 183 | 23.239 | 22.148 | 69.200 | 1.00 | 36.96 | B |
| ATOM | 1318 | CB | GLN | 183 | 22.269 | 23.210 | 69.724 | 1.00 | 38.87 | B |
| ATOM | 1319 | CG | GLN | 183 | 22.925 | 24.543 | 70.024 | 1.00 | 43.04 | B |
| ATOM | 1320 | CD | GLN | 183 | 21.969 | 25.536 | 70.653 | 1.00 | 45.13 | B |
| ATOM | 1321 | OE1 | GLN | 183 | 21.663 | 25.448 | 71.832 | 1.00 | 45.23 | B |
| ATOM | 1322 | NE2 | GLN | 183 | 21.493 | 26.492 | 69.856 | 1.00 | 46.40 | B |
| ATOM | 1323 | C | GLN | 183 | 22.455 | 21.018 | 68.567 | 1.00 | 35.80 | B |
| ATOM | 1324 | O | GLN | 183 | 22.097 | 21.073 | 67.397 | 1.00 | 33.40 | B |
| ATOM | 1325 | N | MET | 184 | 22.165 | 20.005 | 69.367 | 1.00 | 36.43 | B |
| ATOM | 1326 | CA | MET | 184 | 21.450 | 18.840 | 68.877 | 1.00 | 37.65 | B |
| ATOM | 1327 | CB | MET | 184 | 22.322 | 17.610 | 69.118 | 1.00 | 38.53 | B |
| ATOM | 1328 | CG | MET | 184 | 22.033 | 16.445 | 68.221 | 1.00 | 41.45 | B |
| ATOM | 1329 | SD | MET | 184 | 23.141 | 15.085 | 68.586 | 1.00 | 42.59 | B |
| ATOM | 1330 | CE | MET | 184 | 22.590 | 14.660 | 70.190 | 1.00 | 40.16 | B |
| ATOM | 1331 | C | MET | 184 | 20.111 | 18.692 | 69.590 | 1.00 | 37.82 | B |
| ATOM | 1332 | O | MET | 184 | 20.021 | 18.909 | 70.790 | 1.00 | 37.22 | B |
| ATOM | 1333 | N | PHE | 185 | 19.070 | 18.328 | 68.844 | 1.00 | 39.01 | B |
| ATOM | 1334 | CA | PHE | 185 | 17.741 | 18.148 | 69.432 | 1.00 | 41.26 | B |
| ATOM | 1335 | CB | PHE | 185 | 16.851 | 19.377 | 69.160 | 1.00 | 40.10 | B |
| ATOM | 1336 | CG | PHE | 185 | 17.499 | 20.697 | 69.494 | 1.00 | 38.50 | B |
| ATOM | 1337 | CD1 | PHE | 185 | 18.249 | 21.377 | 68.544 | 1.00 | 36.52 | B |
| ATOM | 1338 | CD2 | PHE | 185 | 17.376 | 21.248 | 70.770 | 1.00 | 38.29 | B |
| ATOM | 1339 | CE1 | PHE | 185 | 18.869 | 22.586 | 68.851 | 1.00 | 37.06 | B |
| ATOM | 1340 | CE2 | PHE | 185 | 17.994 | 22.459 | 71.089 | 1.00 | 37.60 | B |
| ATOM | 1341 | CZ | PHE | 185 | 18.743 | 23.128 | 70.128 | 1.00 | 37.41 | B |
| ATOM | 1342 | C | PHE | 185 | 17.034 | 16.903 | 68.887 | 1.00 | 43.21 | B |
| ATOM | 1343 | O | PHE | 185 | 17.221 | 16.532 | 67.734 | 1.00 | 41.62 | B |
| ATOM | 1344 | N | ASP | 186 | 16.223 | 16.259 | 69.724 | 1.00 | 46.68 | B |
| ATOM | 1345 | CA | ASP | 186 | 15.482 | 15.078 | 69.286 | 1.00 | 51.00 | B |
| ATOM | 1346 | CB | ASP | 186 | 14.722 | 14.437 | 70.449 | 1.00 | 52.32 | B |
| ATOM | 1347 | CG | ASP | 186 | 15.642 | 13.912 | 71.530 | 1.00 | 54.63 | B |
| ATOM | 1348 | OD1 | ASP | 186 | 16.575 | 13.150 | 71.202 | 1.00 | 55.59 | B |
| ATOM | 1349 | OD2 | ASP | 186 | 15.428 | 14.262 | 72.712 | 1.00 | 56.98 | B |
| ATOM | 1350 | C | ASP | 186 | 14.481 | 15.539 | 68.241 | 1.00 | 52.48 | B |
| ATOM | 1351 | O | ASP | 186 | 13.777 | 16.510 | 68.443 | 1.00 | 52.99 | B |
| ATOM | 1352 | N | ASP | 187 | 14.425 | 14.841 | 67.118 | 1.00 | 55.70 | B |
| ATOM | 1353 | CA | ASP | 187 | 13.500 | 15.214 | 66.061 | 1.00 | 59.24 | B |
| ATOM | 1354 | CB | ASP | 187 | 13.845 | 14.469 | 64.772 | 1.00 | 58.33 | B |
| ATOM | 1355 | CG | ASP | 187 | 13.015 | 14.929 | 63.601 | 1.00 | 58.32 | B |
| ATOM | 1356 | OD1 | ASP | 187 | 13.345 | 14.546 | 62.459 | 1.00 | 59.29 | B |
| ATOM | 1357 | OD2 | ASP | 187 | 12.035 | 15.672 | 63.822 | 1.00 | 58.82 | B |
| ATOM | 1358 | C | ASP | 187 | 12.064 | 14.905 | 66.473 | 1.00 | 61.85 | B |
| ATOM | 1359 | O | ASP | 187 | 11.690 | 13.750 | 66.626 | 1.00 | 62.59 | B |
| ATOM | 1360 | N | PRO | 188 | 11.241 | 15.950 | 66.662 | 1.00 | 64.18 | B |
| ATOM | 1361 | CD | PRO | 188 | 11.573 | 17.374 | 66.493 | 1.00 | 64.61 | B |
| ATOM | 1362 | CA | PRO | 188 | 9.840 | 15.794 | 67.061 | 1.00 | 66.06 | B |
| ATOM | 1363 | CB | PRO | 188 | 9.287 | 17.207 | 66.923 | 1.00 | 65.95 | B |
| ATOM | 1364 | CG | PRO | 188 | 10.472 | 18.048 | 67.271 | 1.00 | 65.81 | B |
| ATOM | 1365 | C | PRO | 188 | 9.094 | 14.793 | 66.189 | 1.00 | 68.16 | B |
| ATOM | 1366 | O | PRO | 188 | 8.316 | 13.981 | 66.687 | 1.00 | 67.45 | B |
| ATOM | 1367 | N | ARG | 189 | 9.345 | 14.854 | 64.886 | 1.00 | 70.27 | B |
| ATOM | 1368 | CA | ARG | 189 | 8.702 | 13.949 | 63.944 | 1.00 | 73.47 | B |
| ATOM | 1369 | CB | ARG | 189 | 9.278 | 14.170 | 62.547 | 1.00 | 73.94 | B |
| ATOM | 1370 | CG | ARG | 189 | 8.869 | 15.498 | 61.926 | 1.00 | 75.92 | B |
| ATOM | 1371 | CD | ARG | 189 | 9.507 | 15.693 | 60.558 | 1.00 | 77.54 | B |
| ATOM | 1372 | NE | ARG | 189 | 10.797 | 16.373 | 60.644 | 1.00 | 78.29 | B |
| ATOM | 1373 | CZ | ARG | 189 | 10.940 | 17.686 | 60.804 | 1.00 | 78.57 | B |
| ATOM | 1374 | NH1 | ARG | 189 | 9.870 | 18.466 | 60.894 | 1.00 | 78.77 | B |
| ATOM | 1375 | NH2 | ARG | 189 | 12.153 | 18.218 | 60.873 | 1.00 | 78.05 | B |
| ATOM | 1376 | C | ARG | 189 | 8.869 | 12.491 | 64.363 | 1.00 | 75.30 | B |
| ATOM | 1377 | O | ARG | 189 | 7.896 | 11.815 | 64.683 | 1.00 | 75.56 | B |
| ATOM | 1378 | N | ASN | 190 | 10.112 | 12.019 | 64.370 | 1.00 | 77.42 | B |
| ATOM | 1379 | CA | ASN | 190 | 10.417 | 10.640 | 64.748 | 1.00 | 78.69 | B |
| ATOM | 1380 | CB | ASN | 190 | 10.760 | 9.829 | 63.494 | 1.00 | 78.94 | B |
| ATOM | 1381 | CG | ASN | 190 | 11.569 | 10.629 | 62.483 | 1.00 | 78.61 | B |
| ATOM | 1382 | OD1 | ASN | 190 | 12.745 | 10.905 | 62.689 | 1.00 | 78.52 | B |
| ATOM | 1383 | ND2 | ASN | 190 | 10.926 | 11.011 | 61.383 | 1.00 | 78.16 | B |
| ATOM | 1384 | C | ASN | 190 | 11.571 | 10.575 | 65.749 | 1.00 | 79.40 | B |
| ATOM | 1385 | O | ASN | 190 | 12.706 | 10.875 | 65.408 | 1.00 | 79.98 | B |
| ATOM | 1386 | N | LYS | 191 | 11.265 | 10.182 | 66.986 | 1.00 | 79.97 | B |
| ATOM | 1387 | CA | LYS | 191 | 12.267 | 10.084 | 68.051 | 1.00 | 79.77 | B |
| ATOM | 1388 | CB | LYS | 191 | 11.616 | 9.561 | 69.336 | 1.00 | 81.11 | B |
| ATOM | 1389 | CG | LYS | 191 | 10.794 | 10.600 | 70.090 | 1.00 | 82.60 | B |
| ATOM | 1390 | CD | LYS | 191 | 11.695 | 11.630 | 70.758 | 1.00 | 83.37 | B |
| ATOM | 1391 | CE | LYS | 191 | 10.887 | 12.716 | 71.450 | 1.00 | 84.12 | B |
| ATOM | 1392 | NZ | LYS | 191 | 10.109 | 13.539 | 70.478 | 1.00 | 84.72 | B |
| ATOM | 1393 | C | LYS | 191 | 13.478 | 9.216 | 67.695 | 1.00 | 78.46 | B |
| ATOM | 1394 | O | LYS | 191 | 14.462 | 9.173 | 68.434 | 1.00 | 77.59 | B |
| ATOM | 1395 | N | ARG | 192 | 13.398 | 8.525 | 66.563 | 1.00 | 76.93 | B |
| ATOM | 1396 | CA | ARG | 192 | 14.489 | 7.675 | 66.106 | 1.00 | 75.17 | B |
| ATOM | 1397 | CB | ARG | 192 | 13.975 | 6.667 | 65.078 | 1.00 | 77.95 | B |
| ATOM | 1398 | CG | ARG | 192 | 15.041 | 5.708 | 64.573 | 1.00 | 80.81 | B |
| ATOM | 1399 | CD | ARG | 192 | 14.801 | 5.305 | 63.122 | 1.00 | 83.98 | B |
| ATOM | 1400 | NE | ARG | 192 | 14.928 | 6.434 | 62.198 | 1.00 | 86.03 | B |
| ATOM | 1401 | CZ | ARG | 192 | 13.946 | 7.277 | 61.884 | 1.00 | 86.70 | B |
| ATOM | 1402 | NH1 | ARG | 192 | 12.737 | 7.133 | 62.415 | 1.00 | 86.57 | B |
| ATOM | 1403 | NH2 | ARG | 192 | 14.175 | 8.267 | 61.033 | 1.00 | 87.03 | B |
| ATOM | 1404 | C | ARG | 192 | 15.565 | 8.545 | 65.463 | 1.00 | 72.66 | B |
| ATOM | 1405 | O | ARG | 192 | 16.699 | 8.112 | 65.272 | 1.00 | 72.31 | B |
| ATOM | 1406 | N | GLY | 193 | 15.195 | 9.781 | 65.136 | 1.00 | 69.32 | B |
| ATOM | 1407 | CA | GLY | 193 | 16.132 | 10.695 | 64.507 | 1.00 | 63.90 | B |
| ATOM | 1408 | C | GLY | 193 | 16.538 | 11.863 | 65.382 | 1.00 | 59.50 | B |
| ATOM | 1409 | O | GLY | 193 | 16.132 | 11.961 | 66.531 | 1.00 | 59.54 | B |
| ATOM | 1410 | N | VAL | 194 | 17.346 | 12.757 | 64.824 | 1.00 | 55.13 | B |
| ATOM | 1411 | CA | VAL | 194 | 17.812 | 13.918 | 65.562 | 1.00 | 50.91 | B |
| ATOM | 1412 | CB | VAL | 194 | 19.114 | 13.606 | 66.309 | 1.00 | 50.28 | B |
| ATOM | 1413 | CG1 | VAL | 194 | 20.226 | 13.319 | 65.318 | 1.00 | 49.18 | B |
| ATOM | 1414 | CG2 | VAL | 194 | 19.476 | 14.760 | 67.207 | 1.00 | 48.67 | B |
| ATOM | 1415 | C | VAL | 194 | 18.055 | 15.098 | 64.629 | 1.00 | 49.13 | B |
| ATOM | 1416 | O | VAL | 194 | 18.379 | 14.918 | 63.461 | 1.00 | 49.22 | B |
| ATOM | 1417 | N | ILE | 195 | 17.906 | 16.308 | 65.160 | 1.00 | 46.55 | B |
| ATOM | 1418 | CA | ILE | 195 | 18.106 | 17.514 | 64.372 | 1.00 | 42.49 | B |
| ATOM | 1419 | CB | ILE | 195 | 16.846 | 18.405 | 64.396 | 1.00 | 43.57 | B |
| ATOM | 1420 | CG2 | ILE | 195 | 17.076 | 19.653 | 63.561 | 1.00 | 44.86 | B |
| ATOM | 1421 | CG1 | ILE | 195 | 15.647 | 17.639 | 63.837 | 1.00 | 44.25 | B |
| ATOM | 1422 | CD1 | ILE | 195 | 15.828 | 17.184 | 62.393 | 1.00 | 45.64 | B |
| ATOM | 1423 | C | ILE | 195 | 19.291 | 18.349 | 64.856 | 1.00 | 39.72 | B |
| ATOM | 1424 | O | ILE | 195 | 19.379 | 18.691 | 66.030 | 1.00 | 38.69 | B |
| ATOM | 1425 | N | ILE | 196 | 20.197 | 18.672 | 63.936 | 1.00 | 37.40 | B |
| ATOM | 1426 | CA | ILE | 196 | 21.365 | 19.483 | 64.255 | 1.00 | 35.21 | B |
| ATOM | 1427 | CB | ILE | 196 | 22.654 | 18.960 | 63.561 | 1.00 | 34.42 | B |
| ATOM | 1428 | CG2 | ILE | 196 | 23.821 | 19.880 | 63.881 | 1.00 | 33.62 | B |
| ATOM | 1429 | CG1 | ILE | 196 | 23.010 | 17.552 | 64.057 | 1.00 | 33.50 | B |
| ATOM | 1430 | CD1 | ILE | 196 | 22.222 | 16.445 | 63.416 | 1.00 | 31.23 | B |
| ATOM | 1431 | C | ILE | 196 | 21.113 | 20.920 | 63.806 | 1.00 | 35.34 | B |
| ATOM | 1432 | O | ILE | 196 | 21.108 | 21.218 | 62.619 | 1.00 | 33.58 | B |
| ATOM | 1433 | N | LYS | 197 | 20.912 | 21.806 | 64.777 | 1.00 | 36.02 | B |
| ATOM | 1434 | CA | LYS | 197 | 20.639 | 23.209 | 64.494 | 1.00 | 36.95 | B |
| ATOM | 1435 | CB | LYS | 197 | 20.101 | 23.909 | 65.744 | 1.00 | 37.83 | B |
| ATOM | 1436 | CG | LYS | 197 | 19.736 | 25.370 | 65.519 | 1.00 | 42.01 | B |
| ATOM | 1437 | CD | LYS | 197 | 19.391 | 26.055 | 66.829 | 1.00 | 45.50 | B |
| ATOM | 1438 | CE | LYS | 197 | 19.039 | 27.518 | 66.628 | 1.00 | 46.65 | B |
| ATOM | 1439 | NZ | LYS | 197 | 18.686 | 28.161 | 67.932 | 1.00 | 47.32 | B |
| ATOM | 1440 | C | LYS | 197 | 21.857 | 23.968 | 63.983 | 1.00 | 36.01 | B |
| ATOM | 1441 | O | LYS | 197 | 22.887 | 24.025 | 64.646 | 1.00 | 34.47 | B |
| ATOM | 1442 | N | GLY | 198 | 21.722 | 24.547 | 62.793 | 1.00 | 35.82 | B |
| ATOM | 1443 | CA | GLY | 198 | 22.809 | 25.316 | 62.212 | 1.00 | 37.33 | B |
| ATOM | 1444 | C | GLY | 198 | 23.715 | 24.583 | 61.240 | 1.00 | 38.13 | B |
| ATOM | 1445 | O | GLY | 198 | 24.580 | 25.198 | 60.615 | 1.00 | 39.69 | B |
| ATOM | 1446 | N | LEU | 199 | 23.530 | 23.275 | 61.098 | 1.00 | 37.34 | B |
| ATOM | 1447 | CA | LEU | 199 | 24.376 | 22.512 | 60.190 | 1.00 | 36.62 | B |
| ATOM | 1448 | CB | LEU | 199 | 24.218 | 21.006 | 60.444 | 1.00 | 34.70 | B |
| ATOM | 1449 | CG | LEU | 199 | 25.067 | 20.058 | 59.588 | 1.00 | 33.44 | B |
| ATOM | 1450 | CD1 | LEU | 199 | 26.553 | 20.355 | 59.755 | 1.00 | 31.11 | B |
| ATOM | 1451 | CD2 | LEU | 199 | 24.767 | 18.634 | 59.994 | 1.00 | 32.49 | B |
| ATOM | 1452 | C | LEU | 199 | 24.066 | 22.838 | 58.729 | 1.00 | 36.33 | B |
| ATOM | 1453 | O | LEU | 199 | 22.971 | 22.550 | 58.228 | 1.00 | 35.86 | B |
| ATOM | 1454 | N | GLU | 200 | 25.040 | 23.441 | 58.053 | 1.00 | 35.51 | B |
| ATOM | 1455 | CA | GLU | 200 | 24.896 | 23.815 | 56.653 | 1.00 | 37.46 | B |
| ATOM | 1456 | CB | GLU | 200 | 26.037 | 24.746 | 56.234 | 1.00 | 40.69 | B |
| ATOM | 1457 | CG | GLU | 200 | 26.005 | 26.135 | 56.868 | 1.00 | 49.20 | B |
| ATOM | 1458 | CD | GLU | 200 | 24.757 | 26.925 | 56.502 | 1.00 | 51.96 | B |
| ATOM | 1459 | OE1 | GLU | 200 | 23.659 | 26.576 | 56.990 | 1.00 | 54.11 | B |
| ATOM | 1460 | OE2 | GLU | 200 | 24.873 | 27.896 | 55.722 | 1.00 | 54.04 | B |
| ATOM | 1461 | C | GLU | 200 | 24.874 | 22.612 | 55.717 | 1.00 | 36.14 | B |
| ATOM | 1462 | O | GLU | 200 | 25.434 | 21.564 | 56.015 | 1.00 | 35.01 | B |
| ATOM | 1463 | N | GLU | 201 | 24.217 | 22.787 | 54.575 | 1.00 | 35.47 | B |
| ATOM | 1464 | CA | GLU | 201 | 24.124 | 21.752 | 53.559 | 1.00 | 34.36 | B |
| ATOM | 1465 | CB | GLU | 201 | 22.709 | 21.189 | 53.483 | 1.00 | 34.40 | B |
| ATOM | 1466 | CG | GLU | 201 | 22.207 | 20.582 | 54.773 | 1.00 | 34.93 | B |
| ATOM | 1467 | CD | GLU | 201 | 20.816 | 19.998 | 54.626 | 1.00 | 36.86 | B |
| ATOM | 1468 | OE1 | GLU | 201 | 20.137 | 19.825 | 55.665 | 1.00 | 37.44 | B |
| ATOM | 1469 | OE2 | GLU | 201 | 20.408 | 19.710 | 53.476 | 1.00 | 36.10 | B |
| ATOM | 1470 | C | GLU | 201 | 24.479 | 22.393 | 52.226 | 1.00 | 34.09 | B |
| ATOM | 1471 | O | GLU | 201 | 23.681 | 23.115 | 51.657 | 1.00 | 33.70 | B |
| ATOM | 1472 | N | ILE | 202 | 25.687 | 22.127 | 51.740 | 1.00 | 33.17 | B |
| ATOM | 1473 | CA | ILE | 202 | 26.130 | 22.689 | 50.472 | 1.00 | 32.42 | B |
| ATOM | 1474 | CB | ILE | 202 | 27.679 | 22.715 | 50.357 | 1.00 | 33.25 | B |
| ATOM | 1475 | CG2 | ILE | 202 | 28.087 | 23.275 | 49.002 | 1.00 | 31.31 | B |
| ATOM | 1476 | CG1 | ILE | 202 | 28.286 | 23.582 | 51.465 | 1.00 | 33.81 | B |
| ATOM | 1477 | CD1 | ILE | 202 | 28.222 | 22.967 | 52.849 | 1.00 | 36.54 | B |
| ATOM | 1478 | C | ILE | 202 | 25.572 | 21.888 | 49.305 | 1.00 | 31.15 | B |
| ATOM | 1479 | O | ILE | 202 | 25.703 | 20.678 | 49.257 | 1.00 | 33.14 | B |
| ATOM | 1480 | N | THR | 203 | 24.948 | 22.583 | 48.361 | 1.00 | 29.99 | B |
| ATOM | 1481 | CA | THR | 203 | 24.371 | 21.944 | 47.185 | 1.00 | 27.86 | B |
| ATOM | 1482 | CB | THR | 203 | 23.228 | 22.804 | 46.572 | 1.00 | 27.52 | B |
| ATOM | 1483 | OG1 | THR | 203 | 22.157 | 22.925 | 47.516 | 1.00 | 27.78 | B |
| ATOM | 1484 | CG2 | THR | 203 | 22.701 | 22.174 | 45.284 | 1.00 | 26.79 | B |
| ATOM | 1485 | C | THR | 203 | 25.448 | 21.741 | 46.130 | 1.00 | 27.11 | B |
| ATOM | 1486 | O | THR | 203 | 26.217 | 22.637 | 45.853 | 1.00 | 26.94 | B |
| ATOM | 1487 | N | VAL | 204 | 25.500 | 20.541 | 45.560 | 1.00 | 27.55 | B |
| ATOM | 1488 | CA | VAL | 204 | 26.467 | 20.222 | 44.517 | 1.00 | 27.42 | B |
| ATOM | 1489 | CB | VAL | 204 | 27.136 | 18.859 | 44.781 | 1.00 | 25.01 | B |
| ATOM | 1490 | CG1 | VAL | 204 | 28.393 | 18.718 | 43.941 | 1.00 | 23.11 | B |
| ATOM | 1491 | CG2 | VAL | 204 | 27.468 | 18.729 | 46.250 | 1.00 | 23.76 | B |
| ATOM | 1492 | C | VAL | 204 | 25.677 | 20.178 | 43.207 | 1.00 | 29.81 | B |
| ATOM | 1493 | O | VAL | 204 | 24.887 | 19.261 | 42.983 | 1.00 | 30.56 | B |
| ATOM | 1494 | N | HIS | 205 | 25.891 | 21.188 | 42.364 | 1.00 | 30.97 | B |
| ATOM | 1495 | CA | HIS | 205 | 25.197 | 21.318 | 41.079 | 1.00 | 33.24 | B |
| ATOM | 1496 | CB | HIS | 205 | 25.199 | 22.792 | 40.649 | 1.00 | 33.42 | B |
| ATOM | 1497 | CG | HIS | 205 | 24.641 | 23.716 | 41.687 | 1.00 | 34.00 | B |
| ATOM | 1498 | CD2 | HIS | 205 | 25.233 | 24.333 | 42.739 | 1.00 | 33.05 | B |
| ATOM | 1499 | ND1 | HIS | 205 | 23.297 | 24.019 | 41.771 | 1.00 | 33.23 | B |
| ATOM | 1500 | CE1 | HIS | 205 | 23.086 | 24.777 | 42.832 | 1.00 | 33.03 | B |
| ATOM | 1501 | NE2 | HIS | 205 | 24.244 | 24.981 | 43.437 | 1.00 | 32.48 | B |
| ATOM | 1502 | C | HIS | 205 | 25.790 | 20.450 | 39.969 | 1.00 | 33.72 | B |
| ATOM | 1503 | O | HIS | 205 | 25.084 | 20.022 | 39.061 | 1.00 | 32.22 | B |
| ATOM | 1504 | N | ASN | 206 | 27.094 | 20.201 | 40.048 | 1.00 | 35.23 | B |
| ATOM | 1505 | CA | ASN | 206 | 27.779 | 19.381 | 39.055 | 1.00 | 36.89 | B |
| ATOM | 1506 | CB | ASN | 206 | 28.178 | 20.229 | 37.837 | 1.00 | 37.95 | B |
| ATOM | 1507 | CG | ASN | 206 | 28.999 | 21.455 | 38.213 | 1.00 | 41.34 | B |
| ATOM | 1508 | OD1 | ASN | 206 | 30.130 | 21.339 | 38.697 | 1.00 | 43.10 | B |
| ATOM | 1509 | ND2 | ASN | 206 | 28.428 | 22.641 | 37.993 | 1.00 | 38.53 | B |
| ATOM | 1510 | C | ASN | 206 | 29.007 | 18.712 | 39.666 | 1.00 | 36.43 | B |
| ATOM | 1511 | O | ASN | 206 | 29.233 | 18.805 | 40.864 | 1.00 | 36.95 | B |
| ATOM | 1512 | N | LYS | 207 | 29.787 | 18.029 | 38.834 | 1.00 | 36.70 | B |
| ATOM | 1513 | CA | LYS | 207 | 30.983 | 17.338 | 39.297 | 1.00 | 37.65 | B |
| ATOM | 1514 | CB | LYS | 207 | 31.357 | 16.232 | 38.314 | 1.00 | 38.65 | B |
| ATOM | 1515 | CG | LYS | 207 | 31.892 | 16.726 | 36.977 | 1.00 | 41.42 | B |
| ATOM | 1516 | CD | LYS | 207 | 31.938 | 15.585 | 35.966 | 1.00 | 45.62 | B |
| ATOM | 1517 | CE | LYS | 207 | 32.889 | 15.877 | 34.814 | 1.00 | 47.44 | B |
| ATOM | 1518 | NZ | LYS | 207 | 34.314 | 15.937 | 35.262 | 1.00 | 47.37 | B |
| ATOM | 1519 | C | LYS | 207 | 32.155 | 18.298 | 39.464 | 1.00 | 38.02 | B |
| ATOM | 1520 | O | LYS | 207 | 32.990 | 18.121 | 40.340 | 1.00 | 38.46 | B |
| ATOM | 1521 | N | ASP | 208 | 32.199 | 19.320 | 38.618 | 1.00 | 38.91 | B |
| ATOM | 1522 | CA | ASP | 208 | 33.264 | 20.313 | 38.667 | 1.00 | 40.47 | B |
| ATOM | 1523 | CB | ASP | 208 | 33.316 | 21.061 | 37.338 | 1.00 | 42.51 | B |
| ATOM | 1524 | CG | ASP | 208 | 33.664 | 20.156 | 36.192 | 1.00 | 44.26 | B |
| ATOM | 1525 | OD1 | ASP | 208 | 33.297 | 20.470 | 35.041 | 1.00 | 44.33 | B |
| ATOM | 1526 | OD2 | ASP | 208 | 34.321 | 19.127 | 36.451 | 1.00 | 46.27 | B |
| ATOM | 1527 | C | ASP | 208 | 33.058 | 21.300 | 39.805 | 1.00 | 39.34 | B |
| ATOM | 1528 | O | ASP | 208 | 33.568 | 22.405 | 39.780 | 1.00 | 40.79 | B |
| ATOM | 1529 | N | GLU | 209 | 32.308 | 20.893 | 40.813 | 1.00 | 38.81 | B |
| ATOM | 1530 | CA | GLU | 209 | 32.050 | 21.772 | 41.930 | 1.00 | 38.33 | B |
| ATOM | 1531 | CB | GLU | 209 | 30.604 | 22.260 | 41.866 | 1.00 | 39.47 | B |
| ATOM | 1532 | CG | GLU | 209 | 30.278 | 23.400 | 42.805 | 1.00 | 42.87 | B |
| ATOM | 1533 | CD | GLU | 209 | 28.824 | 23.836 | 42.700 | 1.00 | 44.43 | B |
| ATOM | 1534 | OE1 | GLU | 209 | 28.373 | 24.134 | 41.573 | 1.00 | 42.49 | B |
| ATOM | 1535 | OE2 | GLU | 209 | 28.135 | 23.885 | 43.749 | 1.00 | 44.53 | B |
| ATOM | 1536 | C | GLU | 209 | 32.303 | 21.055 | 43.247 | 1.00 | 37.83 | B |
| ATOM | 1537 | O | GLU | 209 | 32.147 | 21.649 | 44.316 | 1.00 | 38.61 | B |
| ATOM | 1538 | N | VAL | 210 | 32.720 | 19.790 | 43.171 | 1.00 | 35.54 | B |
| ATOM | 1539 | CA | VAL | 210 | 32.954 | 19.011 | 44.384 | 1.00 | 32.37 | B |
| ATOM | 1540 | CB | VAL | 210 | 32.679 | 17.485 | 44.158 | 1.00 | 31.94 | B |
| ATOM | 1541 | CG1 | VAL | 210 | 31.641 | 17.286 | 43.057 | 1.00 | 31.12 | B |
| ATOM | 1542 | CG2 | VAL | 210 | 33.961 | 16.749 | 43.842 | 1.00 | 30.76 | B |
| ATOM | 1543 | C | VAL | 210 | 34.342 | 19.173 | 44.991 | 1.00 | 29.97 | B |
| ATOM | 1544 | O | VAL | 210 | 34.482 | 19.206 | 46.207 | 1.00 | 29.98 | B |
| ATOM | 1545 | N | TYR | 211 | 35.367 | 19.285 | 44.154 | 1.00 | 27.29 | B |
| ATOM | 1546 | CA | TYR | 211 | 36.718 | 19.408 | 44.685 | 1.00 | 25.19 | B |
| ATOM | 1547 | CB | TYR | 211 | 37.747 | 19.437 | 43.549 | 1.00 | 24.73 | B |
| ATOM | 1548 | CG | TYR | 211 | 39.177 | 19.352 | 44.040 | 1.00 | 26.20 | B |
| ATOM | 1549 | CD1 | TYR | 211 | 39.601 | 18.278 | 44.824 | 1.00 | 27.98 | B |
| ATOM | 1550 | CE1 | TYR | 211 | 40.903 | 18.214 | 45.325 | 1.00 | 27.65 | B |
| ATOM | 1551 | CD2 | TYR | 211 | 40.093 | 20.360 | 43.761 | 1.00 | 26.06 | B |
| ATOM | 1552 | CE2 | TYR | 211 | 41.398 | 20.308 | 44.257 | 1.00 | 26.72 | B |
| ATOM | 1553 | CZ | TYR | 211 | 41.797 | 19.233 | 45.041 | 1.00 | 29.28 | B |
| ATOM | 1554 | OH | TYR | 211 | 43.081 | 19.193 | 45.556 | 1.00 | 27.76 | B |
| ATOM | 1555 | C | TYR | 211 | 36.864 | 20.635 | 45.573 | 1.00 | 24.67 | B |
| ATOM | 1556 | O | TYR | 211 | 37.515 | 20.578 | 46.615 | 1.00 | 24.02 | B |
| ATOM | 1557 | N | GLN | 212 | 36.251 | 21.742 | 45.160 | 1.00 | 25.05 | B |
| ATOM | 1558 | CA | GLN | 212 | 36.294 | 22.982 | 45.926 | 1.00 | 24.24 | B |
| ATOM | 1559 | CB | GLN | 212 | 35.508 | 24.082 | 45.224 | 1.00 | 27.89 | B |
| ATOM | 1560 | CG | GLN | 212 | 36.375 | 25.051 | 44.459 | 1.00 | 36.14 | B |
| ATOM | 1561 | CD | GLN | 212 | 35.625 | 26.311 | 44.048 | 1.00 | 40.99 | B |
| ATOM | 1562 | OE1 | GLN | 212 | 34.641 | 26.248 | 43.312 | 1.00 | 42.51 | B |
| ATOM | 1563 | NE2 | GLN | 212 | 36.090 | 27.465 | 44.532 | 1.00 | 41.52 | B |
| ATOM | 1564 | C | GLN | 212 | 35.713 | 22.777 | 47.305 | 1.00 | 22.91 | B |
| ATOM | 1565 | O | GLN | 212 | 36.285 | 23.206 | 48.299 | 1.00 | 23.35 | B |
| ATOM | 1566 | N | ILE | 213 | 34.560 | 22.122 | 47.362 | 1.00 | 22.44 | B |
| ATOM | 1567 | CA | ILE | 213 | 33.905 | 21.876 | 48.640 | 1.00 | 22.31 | B |
| ATOM | 1568 | CB | ILE | 213 | 32.595 | 21.095 | 48.472 | 1.00 | 20.76 | B |
| ATOM | 1569 | CG2 | ILE | 213 | 31.910 | 20.947 | 49.828 | 1.00 | 21.01 | B |
| ATOM | 1570 | CG1 | ILE | 213 | 31.675 | 21.821 | 47.492 | 1.00 | 20.79 | B |
| ATOM | 1571 | CD1 | ILE | 213 | 30.457 | 21.012 | 47.071 | 1.00 | 22.47 | B |
| ATOM | 1572 | C | ILE | 213 | 34.816 | 21.095 | 49.573 | 1.00 | 22.67 | B |
| ATOM | 1573 | O | ILE | 213 | 34.863 | 21.366 | 50.764 | 1.00 | 23.38 | B |
| ATOM | 1574 | N | LEU | 214 | 35.539 | 20.126 | 49.020 | 1.00 | 24.93 | B |
| ATOM | 1575 | CA | LEU | 214 | 36.455 | 19.307 | 49.811 | 1.00 | 26.22 | B |
| ATOM | 1576 | CB | LEU | 214 | 36.965 | 18.129 | 48.972 | 1.00 | 27.09 | B |
| ATOM | 1577 | CG | LEU | 214 | 36.092 | 16.868 | 48.882 | 1.00 | 29.34 | B |
| ATOM | 1578 | CD1 | LEU | 214 | 34.618 | 17.235 | 48.836 | 1.00 | 30.24 | B |
| ATOM | 1579 | CD2 | LEU | 214 | 36.491 | 16.059 | 47.649 | 1.00 | 30.55 | B |
| ATOM | 1580 | C | LEU | 214 | 37.621 | 20.149 | 50.314 | 1.00 | 26.01 | B |
| ATOM | 1581 | O | LEU | 214 | 38.064 | 19.994 | 51.444 | 1.00 | 26.33 | B |
| ATOM | 1582 | N | GLU | 215 | 38.108 | 21.049 | 49.464 | 1.00 | 25.83 | B |
| ATOM | 1583 | CA | GLU | 215 | 39.215 | 21.930 | 49.834 | 1.00 | 24.69 | B |
| ATOM | 1584 | CB | GLU | 215 | 39.586 | 22.830 | 48.655 | 1.00 | 23.60 | B |
| ATOM | 1585 | CG | GLU | 215 | 40.814 | 22.380 | 47.882 | 1.00 | 22.50 | B |
| ATOM | 1586 | CD | GLU | 215 | 40.907 | 23.030 | 46.511 | 1.00 | 23.11 | B |
| ATOM | 1587 | OE1 | GLU | 215 | 42.047 | 23.251 | 46.040 | 1.00 | 20.98 | B |
| ATOM | 1588 | OE2 | GLU | 215 | 39.839 | 23.306 | 45.913 | 1.00 | 20.38 | B |
| ATOM | 1589 | C | GLU | 215 | 38.837 | 22.784 | 51.040 | 1.00 | 23.82 | B |
| ATOM | 1590 | O | GLU | 215 | 39.636 | 22.960 | 51.967 | 1.00 | 23.91 | B |
| ATOM | 1591 | N | LYS | 216 | 37.617 | 23.306 | 51.033 | 1.00 | 22.14 | B |
| ATOM | 1592 | CA | LYS | 216 | 37.152 | 24.135 | 52.129 | 1.00 | 24.81 | B |
| ATOM | 1593 | CB | LYS | 216 | 35.794 | 24.747 | 51.781 | 1.00 | 28.88 | B |
| ATOM | 1594 | CG | LYS | 216 | 35.875 | 25.760 | 50.637 | 1.00 | 35.31 | B |
| ATOM | 1595 | CD | LYS | 216 | 34.492 | 26.263 | 50.229 | 1.00 | 40.73 | B |
| ATOM | 1596 | CE | LYS | 216 | 34.591 | 27.386 | 49.208 | 1.00 | 42.22 | B |
| ATOM | 1597 | NZ | LYS | 216 | 35.405 | 27.007 | 48.007 | 1.00 | 44.86 | B |
| ATOM | 1598 | C | LYS | 216 | 37.066 | 23.327 | 53.417 | 1.00 | 24.49 | B |
| ATOM | 1599 | O | LYS | 216 | 37.497 | 23.790 | 54.475 | 1.00 | 25.43 | B |
| ATOM | 1600 | N | GLY | 217 | 36.525 | 22.117 | 53.325 | 1.00 | 22.80 | B |
| ATOM | 1601 | CA | GLY | 217 | 36.427 | 21.282 | 54.498 | 1.00 | 21.61 | B |
| ATOM | 1602 | C | GLY | 217 | 37.813 | 21.056 | 55.063 | 1.00 | 21.73 | B |
| ATOM | 1603 | O | GLY | 217 | 38.019 | 21.154 | 56.273 | 1.00 | 21.45 | B |
| ATOM | 1604 | N | ALA | 218 | 38.770 | 20.770 | 54.182 | 1.00 | 19.63 | B |
| ATOM | 1605 | CA | ALA | 218 | 40.146 | 20.522 | 54.607 | 1.00 | 20.23 | B |
| ATOM | 1606 | CB | ALA | 218 | 41.013 | 20.194 | 53.402 | 1.00 | 20.86 | B |
| ATOM | 1607 | C | ALA | 218 | 40.720 | 21.717 | 55.358 | 1.00 | 19.43 | B |
| ATOM | 1608 | O | ALA | 218 | 41.151 | 21.588 | 56.500 | 1.00 | 21.17 | B |
| ATOM | 1609 | N | ALA | 219 | 40.725 | 22.877 | 54.706 | 1.00 | 19.70 | B |
| ATOM | 1610 | CA | ALA | 219 | 41.248 | 24.111 | 55.299 | 1.00 | 18.89 | B |
| ATOM | 1611 | CB | ALA | 219 | 40.928 | 25.296 | 54.400 | 1.00 | 17.46 | B |
| ATOM | 1612 | C | ALA | 219 | 40.672 | 24.357 | 56.675 | 1.00 | 18.82 | B |
| ATOM | 1613 | O | ALA | 219 | 41.394 | 24.630 | 57.621 | 1.00 | 19.06 | B |
| ATOM | 1614 | N | LYS | 220 | 39.355 | 24.266 | 56.778 | 1.00 | 19.83 | B |
| ATOM | 1615 | CA | LYS | 220 | 38.698 | 24.501 | 58.049 | 1.00 | 21.65 | B |
| ATOM | 1616 | CB | LYS | 220 | 37.179 | 24.475 | 57.867 | 1.00 | 22.34 | B |
| ATOM | 1617 | CG | LYS | 220 | 36.416 | 24.906 | 59.101 | 1.00 | 25.89 | B |
| ATOM | 1618 | CD | LYS | 220 | 35.002 | 25.363 | 58.759 | 1.00 | 28.36 | B |
| ATOM | 1619 | CE | LYS | 220 | 34.296 | 25.886 | 60.002 | 1.00 | 28.81 | B |
| ATOM | 1620 | NZ | LYS | 220 | 32.888 | 26.286 | 59.732 | 1.00 | 27.62 | B |
| ATOM | 1621 | C | LYS | 220 | 39.145 | 23.486 | 59.101 | 1.00 | 21.92 | B |
| ATOM | 1622 | O | LYS | 220 | 39.199 | 23.807 | 60.278 | 1.00 | 23.01 | B |
| ATOM | 1623 | N | ARG | 221 | 39.478 | 22.268 | 58.672 | 1.00 | 21.66 | B |
| ATOM | 1624 | CA | ARG | 221 | 39.934 | 21.223 | 59.596 | 1.00 | 20.06 | B |
| ATOM | 1625 | CB | ARG | 221 | 40.015 | 19.878 | 58.882 | 1.00 | 22.12 | B |
| ATOM | 1626 | CG | ARG | 221 | 38.739 | 19.076 | 58.916 | 1.00 | 23.91 | B |
| ATOM | 1627 | CD | ARG | 221 | 38.952 | 17.787 | 58.173 | 1.00 | 26.21 | B |
| ATOM | 1628 | NE | ARG | 221 | 37.777 | 16.929 | 58.203 | 1.00 | 27.96 | B |
| ATOM | 1629 | CZ | ARG | 221 | 37.620 | 15.882 | 57.407 | 1.00 | 27.08 | B |
| ATOM | 1630 | NH1 | ARG | 221 | 38.571 | 15.583 | 56.529 | 1.00 | 25.16 | B |
| ATOM | 1631 | NH2 | ARG | 221 | 36.519 | 15.145 | 57.491 | 1.00 | 27.49 | B |
| ATOM | 1632 | C | ARG | 221 | 41.301 | 21.562 | 60.167 | 1.00 | 18.78 | B |
| ATOM | 1633 | O | ARG | 221 | 41.623 | 21.206 | 61.315 | 1.00 | 16.42 | B |
| ATOM | 1634 | N | THR | 222 | 42.101 | 22.238 | 59.350 | 1.00 | 15.19 | B |
| ATOM | 1635 | CA | THR | 222 | 43.433 | 22.659 | 59.741 | 1.00 | 15.22 | B |
| ATOM | 1636 | CB | THR | 222 | 44.119 | 23.409 | 58.593 | 1.00 | 16.99 | B |
| ATOM | 1637 | OG1 | THR | 222 | 44.121 | 22.573 | 57.424 | 1.00 | 16.46 | B |
| ATOM | 1638 | CG2 | THR | 222 | 45.534 | 23.796 | 58.977 | 1.00 | 14.73 | B |
| ATOM | 1639 | C | THR | 222 | 43.323 | 23.601 | 60.928 | 1.00 | 16.64 | B |
| ATOM | 1640 | O | THR | 222 | 44.046 | 23.461 | 61.920 | 1.00 | 16.06 | B |
| ATOM | 1641 | N | THR | 223 | 42.405 | 24.559 | 60.828 | 1.00 | 16.39 | B |
| ATOM | 1642 | CA | THR | 223 | 42.202 | 25.515 | 61.902 | 1.00 | 17.40 | B |
| ATOM | 1643 | CB | THR | 223 | 41.160 | 26.603 | 61.519 | 1.00 | 18.18 | B |
| ATOM | 1644 | OG1 | THR | 223 | 39.839 | 26.125 | 61.780 | 1.00 | 22.16 | B |
| ATOM | 1645 | CG2 | THR | 223 | 41.268 | 26.953 | 60.048 | 1.00 | 18.76 | B |
| ATOM | 1646 | C | THR | 223 | 41.708 | 24.757 | 63.134 | 1.00 | 17.96 | B |
| ATOM | 1647 | O | THR | 223 | 42.078 | 25.083 | 64.253 | 1.00 | 20.22 | B |
| ATOM | 1648 | N | ALA | 224 | 40.875 | 23.743 | 62.916 | 1.00 | 17.09 | B |
| ATOM | 1649 | CA | ALA | 224 | 40.348 | 22.953 | 64.027 | 1.00 | 17.61 | B |
| ATOM | 1650 | CB | ALA | 224 | 39.349 | 21.902 | 63.520 | 1.00 | 17.42 | B |
| ATOM | 1651 | C | ALA | 224 | 41.503 | 22.268 | 64.744 | 1.00 | 16.75 | B |
| ATOM | 1652 | O | ALA | 224 | 41.588 | 22.284 | 65.979 | 1.00 | 13.71 | B |
| ATOM | 1653 | N | ALA | 225 | 42.384 | 21.663 | 63.950 | 1.00 | 16.23 | B |
| ATOM | 1654 | CA | ALA | 225 | 43.551 | 20.980 | 64.486 | 1.00 | 15.92 | B |
| ATOM | 1655 | CB | ALA | 225 | 44.391 | 20.426 | 63.346 | 1.00 | 14.25 | B |
| ATOM | 1656 | C | ALA | 225 | 44.376 | 21.956 | 65.332 | 1.00 | 16.42 | B |
| ATOM | 1657 | O | ALA | 225 | 44.983 | 21.566 | 66.329 | 1.00 | 14.18 | B |
| ATOM | 1658 | N | THR | 226 | 44.385 | 23.231 | 64.931 | 1.00 | 18.14 | B |
| ATOM | 1659 | CA | THR | 226 | 45.135 | 24.261 | 65.666 | 1.00 | 18.36 | B |
| ATOM | 1660 | CB | THR | 226 | 45.205 | 25.606 | 64.894 | 1.00 | 19.59 | B |
| ATOM | 1661 | OG1 | THR | 226 | 45.994 | 25.445 | 63.705 | 1.00 | 20.89 | B |
| ATOM | 1662 | CG2 | THR | 226 | 45.821 | 26.696 | 65.775 | 1.00 | 18.63 | B |
| ATOM | 1663 | C | THR | 226 | 44.507 | 24.541 | 67.024 | 1.00 | 19.56 | B |
| ATOM | 1664 | O | THR | 226 | 45.214 | 24.765 | 68.000 | 1.00 | 22.00 | B |
| ATOM | 1665 | N | LEU | 227 | 43.178 | 24.527 | 67.074 | 1.00 | 19.70 | B |
| ATOM | 1666 | CA | LEU | 227 | 42.427 | 24.798 | 68.297 | 1.00 | 20.19 | B |
| ATOM | 1667 | CB | LEU | 227 | 41.011 | 25.291 | 67.943 | 1.00 | 22.99 | B |
| ATOM | 1668 | CG | LEU | 227 | 40.728 | 26.794 | 67.875 | 1.00 | 28.11 | B |
| ATOM | 1669 | CD1 | LEU | 227 | 41.162 | 27.422 | 69.202 | 1.00 | 28.40 | B |
| ATOM | 1670 | CD2 | LEU | 227 | 41.452 | 27.445 | 66.677 | 1.00 | 27.33 | B |
| ATOM | 1671 | C | LEU | 227 | 42.279 | 23.627 | 69.269 | 1.00 | 19.64 | B |
| ATOM | 1672 | O | LEU | 227 | 42.384 | 23.801 | 70.480 | 1.00 | 17.11 | B |
| ATOM | 1673 | N | MET | 228 | 42.021 | 22.440 | 68.727 | 1.00 | 21.48 | B |
| ATOM | 1674 | CA | MET | 228 | 41.807 | 21.253 | 69.557 | 1.00 | 21.62 | B |
| ATOM | 1675 | CB | MET | 228 | 40.465 | 20.627 | 69.174 | 1.00 | 21.31 | B |
| ATOM | 1676 | CG | MET | 228 | 39.286 | 21.542 | 69.510 | 1.00 | 22.62 | B |
| ATOM | 1677 | SD | MET | 228 | 37.764 | 21.286 | 68.570 | 1.00 | 28.36 | B |
| ATOM | 1678 | CE | MET | 228 | 37.979 | 22.463 | 67.223 | 1.00 | 25.23 | B |
| ATOM | 1679 | C | MET | 228 | 42.936 | 20.235 | 69.472 | 1.00 | 19.55 | B |
| ATOM | 1680 | O | MET | 228 | 43.364 | 19.884 | 68.392 | 1.00 | 19.08 | B |
| ATOM | 1681 | N | ASN | 229 | 43.404 | 19.764 | 70.628 | 1.00 | 19.30 | B |
| ATOM | 1682 | CA | ASN | 229 | 44.496 | 18.790 | 70.683 | 1.00 | 21.72 | B |
| ATOM | 1683 | CB | ASN | 229 | 44.902 | 18.512 | 72.140 | 1.00 | 21.27 | B |
| ATOM | 1684 | CG | ASN | 229 | 45.124 | 19.786 | 72.952 | 1.00 | 23.92 | B |
| ATOM | 1685 | OD1 | ASN | 229 | 45.493 | 20.829 | 72.413 | 1.00 | 26.36 | B |
| ATOM | 1686 | ND2 | ASN | 229 | 44.913 | 19.694 | 74.262 | 1.00 | 18.44 | B |
| ATOM | 1687 | C | ASN | 229 | 44.165 | 17.460 | 69.993 | 1.00 | 21.18 | B |
| ATOM | 1688 | O | ASN | 229 | 43.071 | 16.927 | 70.153 | 1.00 | 21.11 | B |
| ATOM | 1689 | N | ALA | 230 | 45.129 | 16.945 | 69.231 | 1.00 | 20.55 | B |
| ATOM | 1690 | CA | ALA | 230 | 44.975 | 15.683 | 68.510 | 1.00 | 21.88 | B |
| ATOM | 1691 | CB | ALA | 230 | 45.172 | 14.502 | 69.466 | 1.00 | 22.05 | B |
| ATOM | 1692 | C | ALA | 230 | 43.599 | 15.601 | 67.869 | 1.00 | 21.44 | B |
| ATOM | 1693 | O | ALA | 230 | 42.925 | 14.588 | 67.974 | 1.00 | 23.20 | B |
| ATOM | 1694 | N | TYR | 231 | 43.197 | 16.667 | 67.191 | 1.00 | 20.11 | B |
| ATOM | 1695 | CA | TYR | 231 | 41.878 | 16.708 | 66.568 | 1.00 | 21.54 | B |
| ATOM | 1696 | CB | TYR | 231 | 41.637 | 18.103 | 65.968 | 1.00 | 19.36 | B |
| ATOM | 1697 | CG | TYR | 231 | 40.280 | 18.276 | 65.322 | 1.00 | 14.20 | B |
| ATOM | 1698 | CD1 | TYR | 231 | 40.106 | 18.061 | 63.956 | 1.00 | 10.71 | B |
| ATOM | 1699 | CE1 | TYR | 231 | 38.852 | 18.173 | 63.369 | 1.00 | 9.05 | B |
| ATOM | 1700 | CD2 | TYR | 231 | 39.159 | 18.613 | 66.085 | 1.00 | 14.00 | B |
| ATOM | 1701 | CE2 | TYR | 231 | 37.900 | 18.725 | 65.503 | 1.00 | 11.47 | B |
| ATOM | 1702 | CZ | TYR | 231 | 37.757 | 18.505 | 64.152 | 1.00 | 9.28 | B |
| ATOM | 1703 | OH | TYR | 231 | 36.522 | 18.626 | 63.583 | 1.00 | 11.26 | B |
| ATOM | 1704 | C | TYR | 231 | 41.603 | 15.614 | 65.526 | 1.00 | 22.31 | B |
| ATOM | 1705 | O | TYR | 231 | 40.611 | 14.889 | 65.630 | 1.00 | 23.44 | B |
| ATOM | 1706 | N | SER | 232 | 42.481 | 15.482 | 64.538 | 1.00 | 21.31 | B |
| ATOM | 1707 | CA | SER | 232 | 42.286 | 14.487 | 63.486 | 1.00 | 21.21 | B |
| ATOM | 1708 | CB | SER | 232 | 43.382 | 14.614 | 62.424 | 1.00 | 19.70 | B |
| ATOM | 1709 | OG | SER | 232 | 44.658 | 14.355 | 62.980 | 1.00 | 22.28 | B |
| ATOM | 1710 | C | SER | 232 | 42.245 | 13.046 | 63.983 | 1.00 | 20.84 | B |
| ATOM | 1711 | O | SER | 232 | 41.718 | 12.165 | 63.303 | 1.00 | 21.67 | B |
| ATOM | 1712 | N | SER | 233 | 42.788 | 12.805 | 65.166 | 1.00 | 18.82 | B |
| ATOM | 1713 | CA | SER | 233 | 42.801 | 11.447 | 65.670 | 1.00 | 16.78 | B |
| ATOM | 1714 | CB | SER | 233 | 44.189 | 11.108 | 66.222 | 1.00 | 14.92 | B |
| ATOM | 1715 | OG | SER | 233 | 44.295 | 11.465 | 67.587 | 1.00 | 15.42 | B |
| ATOM | 1716 | C | SER | 233 | 41.745 | 11.193 | 66.741 | 1.00 | 17.60 | B |
| ATOM | 1717 | O | SER | 233 | 41.365 | 10.067 | 66.964 | 1.00 | 18.14 | B |
| ATOM | 1718 | N | ARG | 234 | 41.267 | 12.253 | 67.392 | 1.00 | 18.41 | B |
| ATOM | 1719 | CA | ARG | 234 | 40.266 | 12.113 | 68.450 | 1.00 | 18.22 | B |
| ATOM | 1720 | CB | ARG | 234 | 40.716 | 12.874 | 69.703 | 1.00 | 20.85 | B |
| ATOM | 1721 | CG | ARG | 234 | 41.207 | 11.975 | 70.809 | 1.00 | 26.63 | B |
| ATOM | 1722 | CD | ARG | 234 | 42.603 | 12.340 | 71.282 | 1.00 | 28.86 | B |
| ATOM | 1723 | NE | ARG | 234 | 42.624 | 13.522 | 72.138 | 1.00 | 28.89 | B |
| ATOM | 1724 | CZ | ARG | 234 | 43.641 | 13.853 | 72.927 | 1.00 | 30.32 | B |
| ATOM | 1725 | NH1 | ARG | 234 | 44.724 | 13.089 | 72.969 | 1.00 | 29.87 | B |
| ATOM | 1726 | NH2 | ARG | 234 | 43.571 | 14.941 | 73.683 | 1.00 | 29.28 | B |
| ATOM | 1727 | C | ARG | 234 | 38.858 | 12.559 | 68.065 | 1.00 | 18.79 | B |
| ATOM | 1728 | O | ARG | 234 | 37.986 | 12.639 | 68.914 | 1.00 | 18.55 | B |
| ATOM | 1729 | N | SER | 235 | 38.641 | 12.826 | 66.780 | 1.00 | 19.09 | B |
| ATOM | 1730 | CA | SER | 235 | 37.339 | 13.278 | 66.307 | 1.00 | 18.40 | B |
| ATOM | 1731 | CB | SER | 235 | 37.477 | 14.654 | 65.655 | 1.00 | 16.08 | B |
| ATOM | 1732 | OG | SER | 235 | 38.275 | 14.584 | 64.481 | 1.00 | 13.92 | B |
| ATOM | 1733 | C | SER | 235 | 36.694 | 12.314 | 65.312 | 1.00 | 18.89 | B |
| ATOM | 1734 | O | SER | 235 | 37.379 | 11.637 | 64.558 | 1.00 | 18.57 | B |
| ATOM | 1735 | N | HIS | 236 | 35.363 | 12.284 | 65.323 | 1.00 | 20.05 | B |
| ATOM | 1736 | CA | HIS | 236 | 34.571 | 11.445 | 64.427 | 1.00 | 20.67 | B |
| ATOM | 1737 | CB | HIS | 236 | 33.409 | 10.800 | 65.186 | 1.00 | 21.89 | B |
| ATOM | 1738 | CG | HIS | 236 | 33.819 | 10.092 | 66.439 | 1.00 | 22.09 | B |
| ATOM | 1739 | CD2 | HIS | 236 | 33.733 | 10.462 | 67.740 | 1.00 | 22.95 | B |
| ATOM | 1740 | ND1 | HIS | 236 | 34.406 | 8.847 | 66.433 | 1.00 | 22.44 | B |
| ATOM | 1741 | CE1 | HIS | 236 | 34.663 | 8.480 | 67.677 | 1.00 | 24.61 | B |
| ATOM | 1742 | NE2 | HIS | 236 | 34.265 | 9.441 | 68.489 | 1.00 | 23.56 | B |
| ATOM | 1743 | C | HIS | 236 | 33.994 | 12.353 | 63.345 | 1.00 | 21.61 | B |
| ATOM | 1744 | O | HIS | 236 | 33.373 | 13.368 | 63.658 | 1.00 | 22.50 | B |
| ATOM | 1745 | N | SER | 237 | 34.195 | 12.000 | 62.080 | 1.00 | 20.87 | B |
| ATOM | 1746 | CA | SER | 237 | 33.673 | 12.813 | 60.992 | 1.00 | 21.41 | B |
| ATOM | 1747 | CB | SER | 237 | 34.811 | 13.241 | 60.061 | 1.00 | 21.79 | B |
| ATOM | 1748 | OG | SER | 237 | 35.388 | 12.121 | 59.411 | 1.00 | 21.23 | B |
| ATOM | 1749 | C | SER | 237 | 32.618 | 12.049 | 60.201 | 1.00 | 22.61 | B |
| ATOM | 1750 | O | SER | 237 | 32.863 | 10.939 | 59.749 | 1.00 | 23.35 | B |
| ATOM | 1751 | N | VAL | 238 | 31.440 | 12.648 | 60.053 | 1.00 | 21.59 | B |
| ATOM | 1752 | CA | VAL | 238 | 30.348 | 12.022 | 59.313 | 1.00 | 20.89 | B |
| ATOM | 1753 | CB | VAL | 238 | 29.106 | 11.821 | 60.234 | 1.00 | 22.16 | B |
| ATOM | 1754 | CG1 | VAL | 238 | 28.807 | 13.104 | 60.977 | 1.00 | 24.21 | B |
| ATOM | 1755 | CG2 | VAL | 238 | 27.886 | 11.395 | 59.419 | 1.00 | 18.41 | B |
| ATOM | 1756 | C | VAL | 238 | 29.967 | 12.872 | 58.103 | 1.00 | 18.95 | B |
| ATOM | 1757 | O | VAL | 238 | 29.157 | 13.772 | 58.205 | 1.00 | 18.39 | B |
| ATOM | 1758 | N | PHE | 239 | 30.586 | 12.577 | 56.962 | 1.00 | 19.38 | B |
| ATOM | 1759 | CA | PHE | 239 | 30.329 | 13.295 | 55.712 | 1.00 | 19.10 | B |
| ATOM | 1760 | CB | PHE | 239 | 31.501 | 13.115 | 54.735 | 1.00 | 16.63 | B |
| ATOM | 1761 | CG | PHE | 239 | 31.413 | 13.986 | 53.501 | 1.00 | 13.65 | B |
| ATOM | 1762 | CD1 | PHE | 239 | 30.443 | 13.752 | 52.521 | 1.00 | 13.62 | B |
| ATOM | 1763 | CD2 | PHE | 239 | 32.307 | 15.029 | 53.316 | 1.00 | 11.10 | B |
| ATOM | 1764 | CE1 | PHE | 239 | 30.375 | 14.557 | 51.367 | 1.00 | 11.04 | B |
| ATOM | 1765 | CE2 | PHE | 239 | 32.248 | 15.836 | 52.174 | 1.00 | 11.49 | B |
| ATOM | 1766 | CZ | PHE | 239 | 31.281 | 15.598 | 51.196 | 1.00 | 10.13 | B |
| ATOM | 1767 | C | PHE | 239 | 29.072 | 12.709 | 55.089 | 1.00 | 20.70 | B |
| ATOM | 1768 | O | PHE | 239 | 29.088 | 11.581 | 54.635 | 1.00 | 21.65 | B |
| ATOM | 1769 | N | SER | 240 | 27.992 | 13.487 | 55.056 | 1.00 | 19.79 | B |
| ATOM | 1770 | CA | SER | 240 | 26.737 | 12.999 | 54.489 | 1.00 | 20.02 | B |
| ATOM | 1771 | CB | SER | 240 | 25.568 | 13.303 | 55.430 | 1.00 | 17.99 | B |
| ATOM | 1772 | OG | SER | 240 | 25.714 | 12.651 | 56.682 | 1.00 | 13.88 | B |
| ATOM | 1773 | C | SER | 240 | 26.424 | 13.552 | 53.104 | 1.00 | 21.86 | B |
| ATOM | 1774 | O | SER | 240 | 26.721 | 14.684 | 52.796 | 1.00 | 22.91 | B |
| ATOM | 1775 | N | VAL | 241 | 25.818 | 12.720 | 52.271 | 1.00 | 23.30 | B |
| ATOM | 1776 | CA | VAL | 241 | 25.448 | 13.130 | 50.932 | 1.00 | 24.80 | B |
| ATOM | 1777 | CB | VAL | 241 | 26.432 | 12.581 | 49.884 | 1.00 | 24.40 | B |
| ATOM | 1778 | CG1 | VAL | 241 | 26.805 | 11.139 | 50.226 | 1.00 | 26.22 | B |
| ATOM | 1779 | CG2 | VAL | 241 | 25.807 | 12.668 | 48.494 | 1.00 | 19.02 | B |
| ATOM | 1780 | C | VAL | 241 | 24.035 | 12.646 | 50.619 | 1.00 | 26.53 | B |
| ATOM | 1781 | O | VAL | 241 | 23.806 | 11.465 | 50.433 | 1.00 | 27.95 | B |
| ATOM | 1782 | N | THR | 242 | 23.093 | 13.582 | 50.586 | 1.00 | 28.63 | B |
| ATOM | 1783 | CA | THR | 242 | 21.698 | 13.287 | 50.311 | 1.00 | 30.95 | B |
| ATOM | 1784 | CB | THR | 242 | 20.779 | 14.186 | 51.164 | 1.00 | 32.05 | B |
| ATOM | 1785 | OG1 | THR | 242 | 20.997 | 13.901 | 52.555 | 1.00 | 33.54 | B |
| ATOM | 1786 | CG2 | THR | 242 | 19.319 | 13.939 | 50.825 | 1.00 | 34.70 | B |
| ATOM | 1787 | C | THR | 242 | 21.393 | 13.490 | 48.828 | 1.00 | 32.32 | B |
| ATOM | 1788 | O | THR | 242 | 21.845 | 14.451 | 48.213 | 1.00 | 33.97 | B |
| ATOM | 1789 | N | ILE | 243 | 20.628 | 12.573 | 48.250 | 1.00 | 33.03 | B |
| ATOM | 1790 | CA | ILE | 243 | 20.293 | 12.660 | 46.837 | 1.00 | 33.83 | B |
| ATOM | 1791 | CB | ILE | 243 | 20.912 | 11.493 | 46.052 | 1.00 | 33.37 | B |
| ATOM | 1792 | CG2 | ILE | 243 | 20.732 | 11.719 | 44.561 | 1.00 | 32.82 | B |
| ATOM | 1793 | CG1 | ILE | 243 | 22.395 | 11.361 | 46.400 | 1.00 | 34.30 | B |
| ATOM | 1794 | CD1 | ILE | 243 | 23.071 | 10.176 | 45.750 | 1.00 | 35.23 | B |
| ATOM | 1795 | C | ILE | 243 | 18.789 | 12.635 | 46.604 | 1.00 | 35.12 | B |
| ATOM | 1796 | O | ILE | 243 | 18.175 | 11.581 | 46.655 | 1.00 | 34.29 | B |
| ATOM | 1797 | N | HIS | 244 | 18.197 | 13.803 | 46.364 | 1.00 | 37.02 | B |
| ATOM | 1798 | CA | HIS | 244 | 16.766 | 13.878 | 46.097 | 1.00 | 38.10 | B |
| ATOM | 1799 | CB | HIS | 244 | 16.214 | 15.280 | 46.390 | 1.00 | 40.10 | B |
| ATOM | 1800 | CG | HIS | 244 | 16.190 | 15.635 | 47.845 | 1.00 | 42.80 | B |
| ATOM | 1801 | CD2 | HIS | 244 | 15.219 | 15.493 | 48.781 | 1.00 | 43.38 | B |
| ATOM | 1802 | ND1 | HIS | 244 | 17.271 | 16.192 | 48.496 | 1.00 | 44.55 | B |
| ATOM | 1803 | CE1 | HIS | 244 | 16.968 | 16.376 | 49.770 | 1.00 | 44.18 | B |
| ATOM | 1804 | NE2 | HIS | 244 | 15.729 | 15.960 | 49.968 | 1.00 | 43.01 | B |
| ATOM | 1805 | C | HIS | 244 | 16.569 | 13.545 | 44.624 | 1.00 | 38.58 | B |
| ATOM | 1806 | O | HIS | 244 | 17.113 | 14.216 | 43.754 | 1.00 | 38.74 | B |
| ATOM | 1807 | N | MET | 245 | 15.790 | 12.500 | 44.357 | 1.00 | 38.78 | B |
| ATOM | 1808 | CA | MET | 245 | 15.534 | 12.056 | 42.991 | 1.00 | 38.49 | B |
| ATOM | 1809 | CB | MET | 245 | 16.081 | 10.646 | 42.791 | 1.00 | 35.74 | B |
| ATOM | 1810 | CG | MET | 245 | 17.579 | 10.552 | 42.978 | 1.00 | 34.03 | B |
| ATOM | 1811 | SD | MET | 245 | 18.110 | 8.870 | 43.218 | 1.00 | 32.96 | B |
| ATOM | 1812 | CE | MET | 245 | 17.855 | 8.694 | 44.996 | 1.00 | 26.04 | B |
| ATOM | 1813 | C | MET | 245 | 14.058 | 12.083 | 42.618 | 1.00 | 39.24 | B |
| ATOM | 1814 | O | MET | 245 | 13.193 | 11.814 | 43.439 | 1.00 | 39.24 | B |
| ATOM | 1815 | N | LYS | 246 | 13.791 | 12.409 | 41.358 | 1.00 | 39.88 | B |
| ATOM | 1816 | CA | LYS | 246 | 12.430 | 12.477 | 40.855 | 1.00 | 40.90 | B |
| ATOM | 1817 | CB | LYS | 246 | 11.910 | 13.916 | 40.915 | 1.00 | 42.86 | B |
| ATOM | 1818 | CG | LYS | 246 | 10.453 | 14.080 | 40.467 | 1.00 | 45.41 | B |
| ATOM | 1819 | CD | LYS | 246 | 10.140 | 15.516 | 40.018 | 1.00 | 47.23 | B |
| ATOM | 1820 | CE | LYS | 246 | 10.383 | 16.538 | 41.134 | 1.00 | 49.08 | B |
| ATOM | 1821 | NZ | LYS | 246 | 10.267 | 17.954 | 40.659 | 1.00 | 47.64 | B |
| ATOM | 1822 | C | LYS | 246 | 12.406 | 11.994 | 39.414 | 1.00 | 41.15 | B |
| ATOM | 1823 | O | LYS | 246 | 13.084 | 12.547 | 38.552 | 1.00 | 40.37 | B |
| ATOM | 1824 | N | GLU | 247 | 11.622 | 10.954 | 39.163 | 1.00 | 40.39 | B |
| ATOM | 1825 | CA | GLU | 247 | 11.496 | 10.414 | 37.821 | 1.00 | 40.56 | B |
| ATOM | 1826 | CB | GLU | 247 | 12.010 | 8.977 | 37.769 | 1.00 | 39.14 | B |
| ATOM | 1827 | CG | GLU | 247 | 11.479 | 8.090 | 38.866 | 1.00 | 37.23 | B |
| ATOM | 1828 | CD | GLU | 247 | 12.390 | 6.916 | 39.118 | 1.00 | 36.86 | B |
| ATOM | 1829 | OE1 | GLU | 247 | 12.094 | 6.104 | 40.021 | 1.00 | 36.22 | B |
| ATOM | 1830 | OE2 | GLU | 247 | 13.410 | 6.813 | 38.406 | 1.00 | 36.77 | B |
| ATOM | 1831 | C | GLU | 247 | 10.039 | 10.469 | 37.402 | 1.00 | 40.31 | B |
| ATOM | 1832 | O | GLU | 247 | 9.142 | 10.304 | 38.220 | 1.00 | 39.86 | B |
| ATOM | 1833 | N | THR | 248 | 9.820 | 10.720 | 36.117 | 1.00 | 40.83 | B |
| ATOM | 1834 | CA | THR | 248 | 8.480 | 10.826 | 35.569 | 1.00 | 40.95 | B |
| ATOM | 1835 | CB | THR | 248 | 8.339 | 12.123 | 34.736 | 1.00 | 40.97 | B |
| ATOM | 1836 | OG1 | THR | 248 | 8.804 | 13.238 | 35.507 | 1.00 | 41.15 | B |
| ATOM | 1837 | CG2 | THR | 248 | 6.886 | 12.363 | 34.358 | 1.00 | 40.88 | B |
| ATOM | 1838 | C | THR | 248 | 8.143 | 9.625 | 34.690 | 1.00 | 40.36 | B |
| ATOM | 1839 | O | THR | 248 | 8.799 | 9.380 | 33.684 | 1.00 | 40.50 | B |
| ATOM | 1840 | N | THR | 249 | 7.111 | 8.885 | 35.086 | 1.00 | 39.94 | B |
| ATOM | 1841 | CA | THR | 249 | 6.661 | 7.712 | 34.341 | 1.00 | 39.13 | B |
| ATOM | 1842 | CB | THR | 249 | 5.537 | 6.976 | 35.086 | 1.00 | 39.64 | B |
| ATOM | 1843 | OG1 | THR | 249 | 4.307 | 7.686 | 34.897 | 1.00 | 37.39 | B |
| ATOM | 1844 | CG2 | THR | 249 | 5.846 | 6.894 | 36.575 | 1.00 | 38.52 | B |
| ATOM | 1845 | C | THR | 249 | 6.115 | 8.132 | 32.980 | 1.00 | 39.50 | B |
| ATOM | 1846 | O | THR | 249 | 5.943 | 9.311 | 32.713 | 1.00 | 39.71 | B |
| ATOM | 1847 | N | ILE | 250 | 5.841 | 7.148 | 32.129 | 1.00 | 40.73 | B |
| ATOM | 1848 | CA | ILE | 250 | 5.307 | 7.398 | 30.794 | 1.00 | 40.49 | B |
| ATOM | 1849 | CB | ILE | 250 | 5.292 | 6.095 | 29.944 | 1.00 | 37.78 | B |
| ATOM | 1850 | CG2 | ILE | 250 | 4.244 | 5.135 | 30.472 | 1.00 | 37.42 | B |
| ATOM | 1851 | CG1 | ILE | 250 | 4.999 | 6.421 | 28.479 | 1.00 | 35.79 | B |
| ATOM | 1852 | CD1 | ILE | 250 | 5.125 | 5.238 | 27.552 | 1.00 | 33.62 | B |
| ATOM | 1853 | C | ILE | 250 | 3.892 | 7.963 | 30.905 | 1.00 | 42.55 | B |
| ATOM | 1854 | O | ILE | 250 | 3.361 | 8.534 | 29.953 | 1.00 | 43.05 | B |
| ATOM | 1855 | N | ASP | 251 | 3.296 | 7.800 | 32.084 | 1.00 | 44.44 | B |
| ATOM | 1856 | CA | ASP | 251 | 1.947 | 8.286 | 32.357 | 1.00 | 46.93 | B |
| ATOM | 1857 | CB | ASP | 251 | 1.215 | 7.318 | 33.290 | 1.00 | 47.07 | B |
| ATOM | 1858 | CG | ASP | 251 | 0.494 | 6.221 | 32.539 | 1.00 | 47.33 | B |
| ATOM | 1859 | OD1 | ASP | 251 | 0.034 | 5.257 | 33.190 | 1.00 | 47.89 | B |
| ATOM | 1860 | OD2 | ASP | 251 | 0.381 | 6.325 | 31.298 | 1.00 | 45.62 | B |
| ATOM | 1861 | C | ASP | 251 | 1.965 | 9.675 | 32.987 | 1.00 | 48.37 | B |
| ATOM | 1862 | O | ASP | 251 | 0.933 | 10.175 | 33.424 | 1.00 | 49.52 | B |
| ATOM | 1863 | N | GLY | 252 | 3.145 | 10.286 | 33.038 | 1.00 | 49.00 | B |
| ATOM | 1864 | CA | GLY | 252 | 3.275 | 11.612 | 33.609 | 1.00 | 48.84 | B |
| ATOM | 1865 | C | GLY | 252 | 3.432 | 11.634 | 35.117 | 1.00 | 49.43 | B |
| ATOM | 1866 | O | GLY | 252 | 3.856 | 12.638 | 35.675 | 1.00 | 49.95 | B |
| ATOM | 1867 | N | GLU | 253 | 3.093 | 10.538 | 35.787 | 1.00 | 49.54 | B |
| ATOM | 1868 | CA | GLU | 253 | 3.219 | 10.499 | 37.237 | 1.00 | 50.34 | B |
| ATOM | 1869 | CB | GLU | 253 | 2.693 | 9.183 | 37.797 | 1.00 | 51.72 | B |
| ATOM | 1870 | CG | GLU | 253 | 2.753 | 9.136 | 39.309 | 1.00 | 55.44 | B |
| ATOM | 1871 | CD | GLU | 253 | 2.605 | 7.734 | 39.856 | 1.00 | 57.73 | B |
| ATOM | 1872 | OE1 | GLU | 253 | 2.703 | 7.561 | 41.091 | 1.00 | 59.23 | B |
| ATOM | 1873 | OE2 | GLU | 253 | 2.400 | 6.805 | 39.048 | 1.00 | 59.21 | B |
| ATOM | 1874 | C | GLU | 253 | 4.671 | 10.678 | 37.661 | 1.00 | 49.73 | B |
| ATOM | 1875 | O | GLU | 253 | 5.582 | 10.326 | 36.930 | 1.00 | 49.04 | B |
| ATOM | 1876 | N | GLU | 254 | 4.878 | 11.229 | 38.851 | 1.00 | 49.71 | B |
| ATOM | 1877 | CA | GLU | 254 | 6.230 | 11.445 | 39.346 | 1.00 | 50.40 | B |
| ATOM | 1878 | CB | GLU | 254 | 6.452 | 12.927 | 39.629 | 1.00 | 51.91 | B |
| ATOM | 1879 | CG | GLU | 254 | 7.036 | 13.680 | 38.448 | 1.00 | 56.74 | B |
| ATOM | 1880 | CD | GLU | 254 | 6.579 | 15.124 | 38.397 | 1.00 | 59.63 | B |
| ATOM | 1881 | OE1 | GLU | 254 | 6.444 | 15.739 | 39.479 | 1.00 | 61.46 | B |
| ATOM | 1882 | OE2 | GLU | 254 | 6.363 | 15.642 | 37.276 | 1.00 | 60.48 | B |
| ATOM | 1883 | C | GLU | 254 | 6.562 | 10.614 | 40.578 | 1.00 | 48.68 | B |
| ATOM | 1884 | O | GLU | 254 | 5.812 | 10.579 | 41.546 | 1.00 | 47.25 | B |
| ATOM | 1885 | N | LEU | 255 | 7.703 | 9.938 | 40.517 | 1.00 | 47.02 | B |
| ATOM | 1886 | CA | LEU | 255 | 8.157 | 9.094 | 41.609 | 1.00 | 45.92 | B |
| ATOM | 1887 | CB | LEU | 255 | 8.566 | 7.722 | 41.067 | 1.00 | 45.31 | B |
| ATOM | 1888 | CG | LEU | 255 | 7.647 | 7.080 | 40.016 | 1.00 | 44.40 | B |
| ATOM | 1889 | CD1 | LEU | 255 | 8.308 | 5.837 | 39.454 | 1.00 | 43.92 | B |
| ATOM | 1890 | CD2 | LEU | 255 | 6.294 | 6.747 | 40.621 | 1.00 | 43.09 | B |
| ATOM | 1891 | C | LEU | 255 | 9.353 | 9.780 | 42.250 | 1.00 | 46.31 | B |
| ATOM | 1892 | O | LEU | 255 | 10.346 | 10.044 | 41.580 | 1.00 | 46.88 | B |
| ATOM | 1893 | N | VAL | 256 | 9.255 | 10.069 | 43.545 | 1.00 | 46.34 | B |
| ATOM | 1894 | CA | VAL | 256 | 10.343 | 10.739 | 44.254 | 1.00 | 46.32 | B |
| ATOM | 1895 | CB | VAL | 256 | 9.837 | 12.012 | 44.988 | 1.00 | 46.60 | B |
| ATOM | 1896 | CG1 | VAL | 256 | 9.447 | 13.075 | 43.971 | 1.00 | 46.43 | B |
| ATOM | 1897 | CG2 | VAL | 256 | 8.642 | 11.679 | 45.870 | 1.00 | 46.46 | B |
| ATOM | 1898 | C | VAL | 256 | 11.049 | 9.835 | 45.258 | 1.00 | 45.32 | B |
| ATOM | 1899 | O | VAL | 256 | 10.428 | 9.287 | 46.158 | 1.00 | 45.96 | B |
| ATOM | 1900 | N | LYS | 257 | 12.359 | 9.687 | 45.077 | 1.00 | 44.55 | B |
| ATOM | 1901 | CA | LYS | 257 | 13.190 | 8.865 | 45.951 | 1.00 | 42.39 | B |
| ATOM | 1902 | CB | LYS | 257 | 13.997 | 7.852 | 45.133 | 1.00 | 43.00 | B |
| ATOM | 1903 | CG | LYS | 257 | 13.170 | 6.932 | 44.261 | 1.00 | 41.72 | B |
| ATOM | 1904 | CD | LYS | 257 | 14.058 | 6.001 | 43.457 | 1.00 | 38.34 | B |
| ATOM | 1905 | CE | LYS | 257 | 14.956 | 6.771 | 42.514 | 1.00 | 37.62 | B |
| ATOM | 1906 | NZ | LYS | 257 | 15.665 | 5.873 | 41.563 | 1.00 | 37.38 | B |
| ATOM | 1907 | C | LYS | 257 | 14.161 | 9.755 | 46.705 | 1.00 | 40.94 | B |
| ATOM | 1908 | O | LYS | 257 | 14.545 | 10.802 | 46.220 | 1.00 | 42.05 | B |
| ATOM | 1909 | N | ILE | 258 | 14.557 | 9.322 | 47.893 | 1.00 | 38.70 | B |
| ATOM | 1910 | CA | ILE | 258 | 15.498 | 10.082 | 48.699 | 1.00 | 35.70 | B |
| ATOM | 1911 | CB | ILE | 258 | 14.790 | 10.816 | 49.850 | 1.00 | 36.93 | B |
| ATOM | 1912 | CG2 | ILE | 258 | 15.811 | 11.596 | 50.667 | 1.00 | 37.53 | B |
| ATOM | 1913 | CG1 | ILE | 258 | 13.729 | 11.767 | 49.291 | 1.00 | 38.43 | B |
| ATOM | 1914 | CD1 | ILE | 258 | 12.932 | 12.500 | 50.363 | 1.00 | 38.30 | B |
| ATOM | 1915 | C | ILE | 258 | 16.541 | 9.142 | 49.285 | 1.00 | 33.73 | B |
| ATOM | 1916 | O | ILE | 258 | 16.257 | 8.388 | 50.209 | 1.00 | 32.97 | B |
| ATOM | 1917 | N | GLY | 259 | 17.746 | 9.186 | 48.731 | 1.00 | 31.67 | B |
| ATOM | 1918 | CA | GLY | 259 | 18.815 | 8.338 | 49.219 | 1.00 | 30.51 | B |
| ATOM | 1919 | C | GLY | 259 | 19.874 | 9.136 | 49.956 | 1.00 | 29.55 | B |
| ATOM | 1920 | O | GLY | 259 | 20.363 | 10.138 | 49.442 | 1.00 | 30.38 | B |
| ATOM | 1921 | N | LYS | 260 | 20.230 | 8.692 | 51.159 | 1.00 | 27.15 | B |
| ATOM | 1922 | CA | LYS | 260 | 21.239 | 9.377 | 51.958 | 1.00 | 26.83 | B |
| ATOM | 1923 | CB | LYS | 260 | 20.603 | 9.940 | 53.240 | 1.00 | 24.21 | B |
| ATOM | 1924 | CG | LYS | 260 | 21.518 | 10.858 | 54.037 | 1.00 | 19.17 | B |
| ATOM | 1925 | CD | LYS | 260 | 20.833 | 11.362 | 55.289 | 1.00 | 17.68 | B |
| ATOM | 1926 | CE | LYS | 260 | 21.768 | 12.219 | 56.124 | 1.00 | 16.42 | B |
| ATOM | 1927 | NZ | LYS | 260 | 21.115 | 12.662 | 57.378 | 1.00 | 16.56 | B |
| ATOM | 1928 | C | LYS | 260 | 22.394 | 8.437 | 52.318 | 1.00 | 27.97 | B |
| ATOM | 1929 | O | LYS | 260 | 22.184 | 7.357 | 52.864 | 1.00 | 30.85 | B |
| ATOM | 1930 | N | LEU | 261 | 23.616 | 8.859 | 52.011 | 1.00 | 26.40 | B |
| ATOM | 1931 | CA | LEU | 261 | 24.792 | 8.056 | 52.306 | 1.00 | 24.54 | B |
| ATOM | 1932 | CB | LEU | 261 | 25.587 | 7.830 | 51.019 | 1.00 | 23.41 | B |
| ATOM | 1933 | CG | LEU | 261 | 26.989 | 7.243 | 51.175 | 1.00 | 23.40 | B |
| ATOM | 1934 | CD1 | LEU | 261 | 26.922 | 5.920 | 51.941 | 1.00 | 20.72 | B |
| ATOM | 1935 | CD2 | LEU | 261 | 27.599 | 7.045 | 49.798 | 1.00 | 20.51 | B |
| ATOM | 1936 | C | LEU | 261 | 25.685 | 8.715 | 53.362 | 1.00 | 23.98 | B |
| ATOM | 1937 | O | LEU | 261 | 26.117 | 9.836 | 53.198 | 1.00 | 22.95 | B |
| ATOM | 1938 | N | ASN | 262 | 25.953 | 8.000 | 54.448 | 1.00 | 22.99 | B |
| ATOM | 1939 | CA | ASN | 262 | 26.799 | 8.529 | 55.511 | 1.00 | 21.81 | B |
| ATOM | 1940 | CB | ASN | 262 | 26.138 | 8.303 | 56.874 | 1.00 | 19.98 | B |
| ATOM | 1941 | CG | ASN | 262 | 24.730 | 8.872 | 56.945 | 1.00 | 24.40 | B |
| ATOM | 1942 | OD1 | ASN | 262 | 23.770 | 8.135 | 57.124 | 1.00 | 24.74 | B |
| ATOM | 1943 | ND2 | ASN | 262 | 24.606 | 10.189 | 56.807 | 1.00 | 20.69 | B |
| ATOM | 1944 | C | ASN | 262 | 28.192 | 7.879 | 55.494 | 1.00 | 21.73 | B |
| ATOM | 1945 | O | ASN | 262 | 28.314 | 6.680 | 55.589 | 1.00 | 20.91 | B |
| ATOM | 1946 | N | LEU | 263 | 29.238 | 8.691 | 55.348 | 1.00 | 21.87 | B |
| ATOM | 1947 | CA | LEU | 263 | 30.611 | 8.191 | 55.338 | 1.00 | 20.99 | B |
| ATOM | 1948 | CB | LEU | 263 | 31.360 | 8.750 | 54.136 | 1.00 | 19.60 | B |
| ATOM | 1949 | CG | LEU | 263 | 30.578 | 8.470 | 52.856 | 1.00 | 20.68 | B |
| ATOM | 1950 | CD1 | LEU | 263 | 31.187 | 9.220 | 51.710 | 1.00 | 22.18 | B |
| ATOM | 1951 | CD2 | LEU | 263 | 30.557 | 6.972 | 52.584 | 1.00 | 20.91 | B |
| ATOM | 1952 | C | LEU | 263 | 31.262 | 8.650 | 56.630 | 1.00 | 21.08 | B |
| ATOM | 1953 | O | LEU | 263 | 31.631 | 9.793 | 56.753 | 1.00 | 20.87 | B |
| ATOM | 1954 | N | VAL | 264 | 31.397 | 7.734 | 57.586 | 1.00 | 22.31 | B |
| ATOM | 1955 | CA | VAL | 264 | 31.964 | 8.048 | 58.901 | 1.00 | 22.41 | B |
| ATOM | 1956 | CB | VAL | 264 | 31.119 | 7.378 | 60.042 | 1.00 | 22.70 | B |
| ATOM | 1957 | CG1 | VAL | 264 | 31.373 | 8.082 | 61.372 | 1.00 | 22.08 | B |
| ATOM | 1958 | CG2 | VAL | 264 | 29.627 | 7.398 | 59.691 | 1.00 | 23.20 | B |
| ATOM | 1959 | C | VAL | 264 | 33.425 | 7.645 | 59.112 | 1.00 | 23.23 | B |
| ATOM | 1960 | O | VAL | 264 | 33.776 | 6.482 | 58.994 | 1.00 | 25.35 | B |
| ATOM | 1961 | N | ASP | 265 | 34.262 | 8.625 | 59.443 | 1.00 | 23.36 | B |
| ATOM | 1962 | CA | ASP | 265 | 35.683 | 8.397 | 59.709 | 1.00 | 21.00 | B |
| ATOM | 1963 | CB | ASP | 265 | 36.528 | 9.471 | 59.011 | 1.00 | 17.94 | B |
| ATOM | 1964 | CG | ASP | 265 | 38.024 | 9.311 | 59.258 | 1.00 | 18.29 | B |
| ATOM | 1965 | OD1 | ASP | 265 | 38.429 | 8.960 | 60.384 | 1.00 | 17.19 | B |
| ATOM | 1966 | OD2 | ASP | 265 | 38.806 | 9.554 | 58.322 | 1.00 | 15.43 | B |
| ATOM | 1967 | C | ASP | 265 | 35.840 | 8.501 | 61.230 | 1.00 | 21.25 | B |
| ATOM | 1968 | O | ASP | 265 | 36.208 | 9.550 | 61.758 | 1.00 | 22.30 | B |
| ATOM | 1969 | N | LEU | 266 | 35.552 | 7.406 | 61.928 | 1.00 | 19.20 | B |
| ATOM | 1970 | CA | LEU | 266 | 35.636 | 7.387 | 63.387 | 1.00 | 19.48 | B |
| ATOM | 1971 | CB | LEU | 266 | 35.269 | 5.991 | 63.913 | 1.00 | 17.26 | B |
| ATOM | 1972 | CG | LEU | 266 | 33.871 | 5.454 | 63.567 | 1.00 | 18.72 | B |
| ATOM | 1973 | CD1 | LEU | 266 | 33.752 | 4.005 | 64.042 | 1.00 | 15.87 | B |
| ATOM | 1974 | CD2 | LEU | 266 | 32.792 | 6.332 | 64.207 | 1.00 | 17.11 | B |
| ATOM | 1975 | C | LEU | 266 | 37.008 | 7.818 | 63.936 | 1.00 | 17.95 | B |
| ATOM | 1976 | O | LEU | 266 | 37.982 | 7.938 | 63.198 | 1.00 | 16.50 | B |
| ATOM | 1977 | N | ALA | 267 | 37.053 | 8.062 | 65.243 | 1.00 | 16.22 | B |
| ATOM | 1978 | CA | ALA | 267 | 38.284 | 8.458 | 65.920 | 1.00 | 17.36 | B |
| ATOM | 1979 | CB | ALA | 267 | 37.957 | 9.144 | 67.244 | 1.00 | 13.49 | B |
| ATOM | 1980 | C | ALA | 267 | 39.112 | 7.202 | 66.183 | 1.00 | 18.67 | B |
| ATOM | 1981 | O | ALA | 267 | 38.561 | 6.119 | 66.320 | 1.00 | 18.45 | B |
| ATOM | 1982 | N | GLY | 268 | 40.430 | 7.357 | 66.249 | 1.00 | 18.66 | B |
| ATOM | 1983 | CA | GLY | 268 | 41.291 | 6.226 | 66.507 | 1.00 | 20.51 | B |
| ATOM | 1984 | C | GLY | 268 | 40.738 | 5.336 | 67.604 | 1.00 | 22.52 | B |
| ATOM | 1985 | O | GLY | 268 | 40.123 | 5.815 | 68.545 | 1.00 | 22.16 | B |
| ATOM | 1986 | N | SER | 269 | 40.974 | 4.033 | 67.483 | 1.00 | 23.43 | B |
| ATOM | 1987 | CA | SER | 269 | 40.471 | 3.075 | 68.461 | 1.00 | 25.19 | B |
| ATOM | 1988 | CB | SER | 269 | 40.083 | 1.796 | 67.750 | 1.00 | 24.66 | B |
| ATOM | 1989 | OG | SER | 269 | 41.131 | 1.412 | 66.883 | 1.00 | 25.58 | B |
| ATOM | 1990 | C | SER | 269 | 41.446 | 2.739 | 69.584 | 1.00 | 26.21 | B |
| ATOM | 1991 | O | SER | 269 | 41.100 | 1.996 | 70.493 | 1.00 | 24.37 | B |
| ATOM | 1992 | N | GLU | 270 | 42.657 | 3.286 | 69.520 | 1.00 | 28.26 | B |
| ATOM | 1993 | CA | GLU | 270 | 43.664 | 3.029 | 70.546 | 1.00 | 31.89 | B |
| ATOM | 1994 | CB | GLU | 270 | 45.031 | 3.589 | 70.118 | 1.00 | 31.04 | B |
| ATOM | 1995 | CG | GLU | 270 | 45.140 | 5.113 | 70.033 | 1.00 | 28.41 | B |
| ATOM | 1996 | CD | GLU | 270 | 44.679 | 5.680 | 68.701 | 1.00 | 28.74 | B |
| ATOM | 1997 | OE1 | GLU | 270 | 44.875 | 6.895 | 68.471 | 1.00 | 30.30 | B |
| ATOM | 1998 | OE2 | GLU | 270 | 44.129 | 4.921 | 67.884 | 1.00 | 28.84 | B |
| ATOM | 1999 | C | GLU | 270 | 43.262 | 3.618 | 71.904 | 1.00 | 35.40 | B |
| ATOM | 2000 | O | GLU | 270 | 42.847 | 4.770 | 71.993 | 1.00 | 34.74 | B |
| ATOM | 2001 | N | ASN | 271 | 43.378 | 2.798 | 72.950 | 1.00 | 40.25 | B |
| ATOM | 2002 | CA | ASN | 271 | 43.039 | 3.192 | 74.324 | 1.00 | 44.12 | B |
| ATOM | 2003 | CB | ASN | 271 | 41.581 | 3.693 | 74.419 | 1.00 | 45.82 | B |
| ATOM | 2004 | CG | ASN | 271 | 40.546 | 2.600 | 74.147 | 1.00 | 46.03 | B |
| ATOM | 2005 | OD1 | ASN | 271 | 39.347 | 2.845 | 74.224 | 1.00 | 45.22 | B |
| ATOM | 2006 | ND2 | ASN | 271 | 41.011 | 1.395 | 73.829 | 1.00 | 47.11 | B |
| ATOM | 2007 | C | ASN | 271 | 43.246 | 2.039 | 75.307 | 1.00 | 45.92 | B |
| ATOM | 2008 | O | ASN | 271 | 43.668 | 0.938 | 74.922 | 1.00 | 46.63 | B |
| ATOM | 2009 | N | ASN | 287 | 41.544 | 11.757 | 79.480 | 1.00 | 56.32 | B |
| ATOM | 2010 | CA | ASN | 287 | 40.687 | 12.175 | 78.374 | 1.00 | 56.59 | B |
| ATOM | 2011 | CB | ASN | 287 | 41.514 | 12.914 | 77.315 | 1.00 | 58.79 | B |
| ATOM | 2012 | CG | ASN | 287 | 42.376 | 14.006 | 77.912 | 1.00 | 60.93 | B |
| ATOM | 2013 | OD1 | ASN | 287 | 43.344 | 13.729 | 78.617 | 1.00 | 62.31 | B |
| ATOM | 2014 | ND2 | ASN | 287 | 42.024 | 15.259 | 77.637 | 1.00 | 61.77 | B |
| ATOM | 2015 | C | ASN | 287 | 39.995 | 10.965 | 77.736 | 1.00 | 54.81 | B |
| ATOM | 2016 | O | ASN | 287 | 40.651 | 10.079 | 77.181 | 1.00 | 55.49 | B |
| ATOM | 2017 | N | ILE | 288 | 38.667 | 10.940 | 77.811 | 1.00 | 50.95 | B |
| ATOM | 2018 | CA | ILE | 288 | 37.889 | 9.838 | 77.252 | 1.00 | 46.25 | B |
| ATOM | 2019 | CB | ILE | 288 | 36.925 | 9.250 | 78.314 | 1.00 | 48.90 | B |
| ATOM | 2020 | CG2 | ILE | 288 | 37.713 | 8.784 | 79.530 | 1.00 | 49.46 | B |
| ATOM | 2021 | CG1 | ILE | 288 | 35.903 | 10.307 | 78.741 | 1.00 | 49.66 | B |
| ATOM | 2022 | CD1 | ILE | 288 | 34.687 | 9.730 | 79.435 | 1.00 | 51.96 | B |
| ATOM | 2023 | C | ILE | 288 | 37.060 | 10.259 | 76.039 | 1.00 | 40.91 | B |
| ATOM | 2024 | O | ILE | 288 | 36.680 | 11.423 | 75.904 | 1.00 | 41.77 | B |
| ATOM | 2025 | N | ASN | 289 | 36.774 | 9.302 | 75.163 | 1.00 | 32.95 | B |
| ATOM | 2026 | CA | ASN | 289 | 35.979 | 9.582 | 73.976 | 1.00 | 26.09 | B |
| ATOM | 2027 | CB | ASN | 289 | 36.674 | 9.045 | 72.728 | 1.00 | 22.00 | B |
| ATOM | 2028 | CG | ASN | 289 | 36.093 | 9.612 | 71.444 | 1.00 | 19.37 | B |
| ATOM | 2029 | OD1 | ASN | 289 | 36.819 | 9.927 | 70.521 | 1.00 | 19.84 | B |
| ATOM | 2030 | ND2 | ASN | 289 | 34.774 | 9.725 | 71.382 | 1.00 | 17.42 | B |
| ATOM | 2031 | C | ASN | 289 | 34.624 | 8.927 | 74.154 | 1.00 | 22.64 | B |
| ATOM | 2032 | O | ASN | 289 | 34.394 | 7.805 | 73.718 | 1.00 | 22.38 | B |
| ATOM | 2033 | N | GLN | 290 | 33.726 | 9.652 | 74.806 | 1.00 | 20.05 | B |
| ATOM | 2034 | CA | GLN | 290 | 32.386 | 9.166 | 75.085 | 1.00 | 18.94 | B |
| ATOM | 2035 | CB | GLN | 290 | 31.542 | 10.299 | 75.659 | 1.00 | 20.27 | B |
| ATOM | 2036 | CG | GLN | 290 | 30.180 | 9.847 | 76.124 | 1.00 | 20.13 | B |
| ATOM | 2037 | CD | GLN | 290 | 30.273 | 8.777 | 77.182 | 1.00 | 20.41 | B |
| ATOM | 2038 | OE1 | GLN | 290 | 29.311 | 8.067 | 77.441 | 1.00 | 22.39 | B |
| ATOM | 2039 | NE2 | GLN | 290 | 31.435 | 8.662 | 77.806 | 1.00 | 20.99 | B |
| ATOM | 2040 | C | GLN | 290 | 31.652 | 8.526 | 73.899 | 1.00 | 18.42 | B |
| ATOM | 2041 | O | GLN | 290 | 30.945 | 7.543 | 74.068 | 1.00 | 15.37 | B |
| ATOM | 2042 | N | SER | 291 | 31.808 | 9.088 | 72.704 | 1.00 | 19.89 | B |
| ATOM | 2043 | CA | SER | 291 | 31.139 | 8.540 | 71.526 | 1.00 | 21.11 | B |
| ATOM | 2044 | CB | SER | 291 | 31.161 | 9.541 | 70.366 | 1.00 | 22.02 | B |
| ATOM | 2045 | OG | SER | 291 | 30.121 | 10.496 | 70.491 | 1.00 | 23.09 | B |
| ATOM | 2046 | C | SER | 291 | 31.757 | 7.212 | 71.090 | 1.00 | 22.87 | B |
| ATOM | 2047 | O | SER | 291 | 31.051 | 6.294 | 70.681 | 1.00 | 24.87 | B |
| ATOM | 2048 | N | LEU | 292 | 33.074 | 7.107 | 71.187 | 1.00 | 21.56 | B |
| ATOM | 2049 | CA | LEU | 292 | 33.741 | 5.878 | 70.812 | 1.00 | 21.17 | B |
| ATOM | 2050 | CB | LEU | 292 | 35.247 | 6.097 | 70.826 | 1.00 | 18.31 | B |
| ATOM | 2051 | CG | LEU | 292 | 36.074 | 5.053 | 70.089 | 1.00 | 18.27 | B |
| ATOM | 2052 | CD1 | LEU | 292 | 35.653 | 4.994 | 68.625 | 1.00 | 13.66 | B |
| ATOM | 2053 | CD2 | LEU | 292 | 37.548 | 5.418 | 70.218 | 1.00 | 17.97 | B |
| ATOM | 2054 | C | LEU | 292 | 33.345 | 4.785 | 71.818 | 1.00 | 21.64 | B |
| ATOM | 2055 | O | LEU | 292 | 32.914 | 3.703 | 71.454 | 1.00 | 19.24 | B |
| ATOM | 2056 | N | LEU | 293 | 33.481 | 5.100 | 73.098 | 1.00 | 22.14 | B |
| ATOM | 2057 | CA | LEU | 293 | 33.141 | 4.172 | 74.158 | 1.00 | 22.23 | B |
| ATOM | 2058 | CB | LEU | 293 | 33.374 | 4.841 | 75.513 | 1.00 | 22.95 | B |
| ATOM | 2059 | CG | LEU | 293 | 34.479 | 4.277 | 76.408 | 1.00 | 25.37 | B |
| ATOM | 2060 | CD1 | LEU | 293 | 35.684 | 3.860 | 75.597 | 1.00 | 25.32 | B |
| ATOM | 2061 | CD2 | LEU | 293 | 34.851 | 5.345 | 77.431 | 1.00 | 26.42 | B |
| ATOM | 2062 | C | LEU | 293 | 31.689 | 3.713 | 74.046 | 1.00 | 24.05 | B |
| ATOM | 2063 | O | LEU | 293 | 31.373 | 2.552 | 74.304 | 1.00 | 27.12 | B |
| ATOM | 2064 | N | THR | 294 | 30.807 | 4.622 | 73.647 | 1.00 | 23.43 | B |
| ATOM | 2065 | CA | THR | 294 | 29.396 | 4.293 | 73.534 | 1.00 | 22.37 | B |
| ATOM | 2066 | CB | THR | 294 | 28.554 | 5.580 | 73.487 | 1.00 | 22.35 | B |
| ATOM | 2067 | OG1 | THR | 294 | 28.706 | 6.277 | 74.734 | 1.00 | 19.68 | B |
| ATOM | 2068 | CG2 | THR | 294 | 27.090 | 5.275 | 73.270 | 1.00 | 19.85 | B |
| ATOM | 2069 | C | THR | 294 | 29.148 | 3.419 | 72.313 | 1.00 | 23.90 | B |
| ATOM | 2070 | O | THR | 294 | 28.276 | 2.561 | 72.325 | 1.00 | 26.74 | B |
| ATOM | 2071 | N | LEU | 295 | 29.938 | 3.628 | 71.268 | 1.00 | 24.08 | B |
| ATOM | 2072 | CA | LEU | 295 | 29.817 | 2.846 | 70.048 | 1.00 | 24.42 | B |
| ATOM | 2073 | CB | LEU | 295 | 30.822 | 3.332 | 69.004 | 1.00 | 22.92 | B |
| ATOM | 2074 | CG | LEU | 295 | 30.940 | 2.449 | 67.760 | 1.00 | 22.72 | B |
| ATOM | 2075 | CD1 | LEU | 295 | 29.647 | 2.481 | 66.975 | 1.00 | 20.45 | B |
| ATOM | 2076 | CD2 | LEU | 295 | 32.096 | 2.925 | 66.907 | 1.00 | 22.47 | B |
| ATOM | 2077 | C | LEU | 295 | 30.064 | 1.361 | 70.340 | 1.00 | 26.15 | B |
| ATOM | 2078 | O | LEU | 295 | 29.363 | 0.503 | 69.836 | 1.00 | 28.14 | B |
| ATOM | 2079 | N | GLY | 296 | 31.079 | 1.076 | 71.149 | 1.00 | 26.16 | B |
| ATOM | 2080 | CA | GLY | 296 | 31.391 | −0.295 | 71.503 | 1.00 | 25.55 | B |
| ATOM | 2081 | C | GLY | 296 | 30.300 | −0.915 | 72.361 | 1.00 | 25.59 | B |
| ATOM | 2082 | O | GLY | 296 | 29.898 | −2.059 | 72.134 | 1.00 | 26.11 | B |
| ATOM | 2083 | N | ARG | 297 | 29.817 | −0.162 | 73.346 | 1.00 | 22.71 | B |
| ATOM | 2084 | CA | ARG | 297 | 28.760 | −0.660 | 74.217 | 1.00 | 22.15 | B |
| ATOM | 2085 | CB | ARG | 297 | 28.528 | 0.306 | 75.372 | 1.00 | 19.27 | B |
| ATOM | 2086 | CG | ARG | 297 | 29.719 | 0.450 | 76.284 | 1.00 | 20.29 | B |
| ATOM | 2087 | CD | ARG | 297 | 29.456 | 1.467 | 77.372 | 1.00 | 22.43 | B |
| ATOM | 2088 | NE | ARG | 297 | 30.639 | 1.658 | 78.201 | 1.00 | 26.34 | B |
| ATOM | 2089 | CZ | ARG | 297 | 31.226 | 2.833 | 78.407 | 1.00 | 24.22 | B |
| ATOM | 2090 | NH1 | ARG | 297 | 30.729 | 3.921 | 77.838 | 1.00 | 23.11 | B |
| ATOM | 2091 | NH2 | ARG | 297 | 32.306 | 2.918 | 79.178 | 1.00 | 18.73 | B |
| ATOM | 2092 | C | ARG | 297 | 27.449 | −0.876 | 73.452 | 1.00 | 21.70 | B |
| ATOM | 2093 | O | ARG | 297 | 26.634 | −1.674 | 73.844 | 1.00 | 20.12 | B |
| ATOM | 2094 | N | VAL | 298 | 27.255 | −0.138 | 72.362 | 1.00 | 23.14 | B |
| ATOM | 2095 | CA | VAL | 298 | 26.046 | −0.284 | 71.558 | 1.00 | 23.54 | B |
| ATOM | 2096 | CB | VAL | 298 | 25.845 | 0.924 | 70.613 | 1.00 | 22.84 | B |
| ATOM | 2097 | CG1 | VAL | 298 | 24.742 | 0.634 | 69.582 | 1.00 | 18.86 | B |
| ATOM | 2098 | CG2 | VAL | 298 | 25.477 | 2.146 | 71.432 | 1.00 | 19.90 | B |
| ATOM | 2099 | C | VAL | 298 | 26.150 | −1.563 | 70.739 | 1.00 | 25.65 | B |
| ATOM | 2100 | O | VAL | 298 | 25.192 | −2.325 | 70.643 | 1.00 | 27.92 | B |
| ATOM | 2101 | N | ILE | 299 | 27.317 | −1.793 | 70.147 | 1.00 | 25.96 | B |
| ATOM | 2102 | CA | ILE | 299 | 27.516 | −2.992 | 69.354 | 1.00 | 27.94 | B |
| ATOM | 2103 | CB | ILE | 299 | 28.880 | −2.971 | 68.649 | 1.00 | 26.11 | B |
| ATOM | 2104 | CG2 | ILE | 299 | 29.187 | −4.330 | 68.053 | 1.00 | 24.74 | B |
| ATOM | 2105 | CG1 | ILE | 299 | 28.862 | −1.910 | 67.550 | 1.00 | 26.37 | B |
| ATOM | 2106 | CD1 | ILE | 299 | 30.192 | −1.704 | 66.889 | 1.00 | 28.12 | B |
| ATOM | 2107 | C | ILE | 299 | 27.413 | −4.240 | 70.235 | 1.00 | 29.09 | B |
| ATOM | 2108 | O | ILE | 299 | 26.958 | −5.284 | 69.791 | 1.00 | 28.96 | B |
| ATOM | 2109 | N | THR | 300 | 27.829 | −4.112 | 71.490 | 1.00 | 29.82 | B |
| ATOM | 2110 | CA | THR | 300 | 27.771 | −5.213 | 72.440 | 1.00 | 30.01 | B |
| ATOM | 2111 | CB | THR | 300 | 28.561 | −4.877 | 73.706 | 1.00 | 29.27 | B |
| ATOM | 2112 | OG1 | THR | 300 | 29.960 | −4.842 | 73.392 | 1.00 | 30.68 | B |
| ATOM | 2113 | CG2 | THR | 300 | 28.299 | −5.900 | 74.796 | 1.00 | 28.12 | B |
| ATOM | 2114 | C | THR | 300 | 26.330 | −5.517 | 72.821 | 1.00 | 32.39 | B |
| ATOM | 2115 | O | THR | 300 | 25.927 | −6.675 | 72.902 | 1.00 | 33.67 | B |
| ATOM | 2116 | N | ALA | 301 | 25.552 | −4.467 | 73.044 | 1.00 | 32.46 | B |
| ATOM | 2117 | CA | ALA | 301 | 24.157 | −4.631 | 73.414 | 1.00 | 34.19 | B |
| ATOM | 2118 | CB | ALA | 301 | 23.584 | −3.305 | 73.863 | 1.00 | 32.83 | B |
| ATOM | 2119 | C | ALA | 301 | 23.353 | −5.182 | 72.238 | 1.00 | 35.75 | B |
| ATOM | 2120 | O | ALA | 301 | 22.348 | −5.842 | 72.425 | 1.00 | 37.02 | B |
| ATOM | 2121 | N | LEU | 302 | 23.812 | −4.899 | 71.024 | 1.00 | 36.43 | B |
| ATOM | 2122 | CA | LEU | 302 | 23.132 | −5.352 | 69.817 | 1.00 | 38.14 | B |
| ATOM | 2123 | CB | LEU | 302 | 23.549 | −4.488 | 68.622 | 1.00 | 38.00 | B |
| ATOM | 2124 | CG | LEU | 302 | 22.492 | −3.555 | 68.031 | 1.00 | 39.25 | B |
| ATOM | 2125 | CD1 | LEU | 302 | 21.823 | −2.753 | 69.128 | 1.00 | 39.09 | B |
| ATOM | 2126 | CD2 | LEU | 302 | 23.149 | −2.630 | 67.016 | 1.00 | 38.56 | B |
| ATOM | 2127 | C | LEU | 302 | 23.428 | −6.812 | 69.514 | 1.00 | 39.23 | B |
| ATOM | 2128 | O | LEU | 302 | 22.520 | −7.594 | 69.249 | 1.00 | 39.50 | B |
| ATOM | 2129 | N | VAL | 303 | 24.709 | −7.163 | 69.552 | 1.00 | 40.87 | B |
| ATOM | 2130 | CA | VAL | 303 | 25.161 | −8.521 | 69.287 | 1.00 | 42.58 | B |
| ATOM | 2131 | CB | VAL | 303 | 26.706 | −8.605 | 69.331 | 1.00 | 42.52 | B |
| ATOM | 2132 | CG1 | VAL | 303 | 27.155 | −10.051 | 69.270 | 1.00 | 43.58 | B |
| ATOM | 2133 | CG2 | VAL | 303 | 27.301 | −7.824 | 68.167 | 1.00 | 42.05 | B |
| ATOM | 2134 | C | VAL | 303 | 24.579 | −9.496 | 70.306 | 1.00 | 44.19 | B |
| ATOM | 2135 | O | VAL | 303 | 24.048 | −10.538 | 69.941 | 1.00 | 45.04 | B |
| ATOM | 2136 | N | GLU | 304 | 24.685 | −9.145 | 71.584 | 1.00 | 45.93 | B |
| ATOM | 2137 | CA | GLU | 304 | 24.169 | −9.973 | 72.667 | 1.00 | 48.10 | B |
| ATOM | 2138 | CB | GLU | 304 | 24.792 | −9.541 | 73.998 | 1.00 | 47.26 | B |
| ATOM | 2139 | CG | GLU | 304 | 26.305 | −9.707 | 74.041 | 1.00 | 46.33 | B |
| ATOM | 2140 | CD | GLU | 304 | 26.901 | −9.334 | 75.382 | 1.00 | 46.65 | B |
| ATOM | 2141 | OE1 | GLU | 304 | 28.139 | −9.410 | 75.519 | 1.00 | 44.41 | B |
| ATOM | 2142 | OE2 | GLU | 304 | 26.135 | −8.968 | 76.302 | 1.00 | 47.42 | B |
| ATOM | 2143 | C | GLU | 304 | 22.649 | −9.885 | 72.753 | 1.00 | 49.92 | B |
| ATOM | 2144 | O | GLU | 304 | 22.031 | −10.492 | 73.612 | 1.00 | 50.02 | B |
| ATOM | 2145 | N | ARG | 305 | 22.061 | −9.116 | 71.844 | 1.00 | 52.91 | B |
| ATOM | 2146 | CA | ARG | 305 | 20.614 | −8.941 | 71.787 | 1.00 | 56.32 | B |
| ATOM | 2147 | CB | ARG | 305 | 19.952 | −10.251 | 71.357 | 1.00 | 58.76 | B |
| ATOM | 2148 | CG | ARG | 305 | 20.300 | −10.652 | 69.934 | 1.00 | 63.36 | B |
| ATOM | 2149 | CD | ARG | 305 | 19.501 | −11.856 | 69.475 | 1.00 | 68.00 | B |
| ATOM | 2150 | NE | ARG | 305 | 19.718 | −12.133 | 68.057 | 1.00 | 71.78 | B |
| ATOM | 2151 | CZ | ARG | 305 | 19.306 | −11.344 | 67.068 | 1.00 | 73.93 | B |
| ATOM | 2152 | NH1 | ARG | 305 | 18.650 | −10.222 | 67.339 | 1.00 | 74.69 | B |
| ATOM | 2153 | NH2 | ARG | 305 | 19.554 | −11.675 | 65.807 | 1.00 | 75.22 | B |
| ATOM | 2154 | C | ARG | 305 | 19.981 | −8.443 | 73.082 | 1.00 | 56.68 | B |
| ATOM | 2155 | O | ARG | 305 | 18.809 | −8.699 | 73.340 | 1.00 | 56.68 | B |
| ATOM | 2156 | N | THR | 306 | 20.757 | −7.728 | 73.892 | 1.00 | 57.02 | B |
| ATOM | 2157 | CA | THR | 306 | 20.248 | −7.185 | 75.146 | 1.00 | 56.82 | B |
| ATOM | 2158 | CB | THR | 306 | 21.347 | −6.426 | 75.912 | 1.00 | 56.33 | B |
| ATOM | 2159 | OG1 | THR | 306 | 22.482 | −7.281 | 76.095 | 1.00 | 56.76 | B |
| ATOM | 2160 | CG2 | THR | 306 | 20.836 | −5.975 | 77.272 | 1.00 | 56.64 | B |
| ATOM | 2161 | C | THR | 306 | 19.122 | −6.213 | 74.812 | 1.00 | 57.35 | B |
| ATOM | 2162 | O | THR | 306 | 19.239 | −5.421 | 73.881 | 1.00 | 58.12 | B |
| ATOM | 2163 | N | PRO | 307 | 18.011 | −6.268 | 75.564 | 1.00 | 57.68 | B |
| ATOM | 2164 | CD | PRO | 307 | 17.750 | −7.184 | 76.688 | 1.00 | 58.36 | B |
| ATOM | 2165 | CA | PRO | 307 | 16.861 | −5.384 | 75.336 | 1.00 | 57.69 | B |
| ATOM | 2166 | CB | PRO | 307 | 15.959 | −5.682 | 76.533 | 1.00 | 57.98 | B |
| ATOM | 2167 | CG | PRO | 307 | 16.241 | −7.125 | 76.803 | 1.00 | 58.68 | B |
| ATOM | 2168 | C | PRO | 307 | 17.218 | −3.898 | 75.237 | 1.00 | 56.99 | B |
| ATOM | 2169 | O | PRO | 307 | 16.684 | −3.187 | 74.386 | 1.00 | 57.64 | B |
| ATOM | 2170 | N | HIS | 308 | 18.120 | −3.439 | 76.105 | 1.00 | 55.27 | B |
| ATOM | 2171 | CA | HIS | 308 | 18.539 | −2.034 | 76.123 | 1.00 | 53.51 | B |
| ATOM | 2172 | CB | HIS | 308 | 18.749 | −1.565 | 77.567 | 1.00 | 55.71 | B |
| ATOM | 2173 | CG | HIS | 308 | 19.227 | −0.150 | 77.677 | 1.00 | 58.12 | B |
| ATOM | 2174 | CD2 | HIS | 308 | 20.385 | 0.367 | 78.155 | 1.00 | 59.12 | B |
| ATOM | 2175 | ND1 | HIS | 308 | 18.475 | 0.925 | 77.252 | 1.00 | 58.97 | B |
| ATOM | 2176 | CE1 | HIS | 308 | 19.148 | 2.043 | 77.464 | 1.00 | 58.91 | B |
| ATOM | 2177 | NE2 | HIS | 308 | 20.310 | 1.732 | 78.012 | 1.00 | 59.24 | B |
| ATOM | 2178 | C | HIS | 308 | 19.813 | −1.749 | 75.329 | 1.00 | 50.82 | B |
| ATOM | 2179 | O | HIS | 308 | 20.793 | −2.472 | 75.433 | 1.00 | 50.26 | B |
| ATOM | 2180 | N | VAL | 309 | 19.780 | −0.671 | 74.551 | 1.00 | 47.79 | B |
| ATOM | 2181 | CA | VAL | 309 | 20.921 | −0.239 | 73.743 | 1.00 | 44.18 | B |
| ATOM | 2182 | CB | VAL | 309 | 20.619 | −0.355 | 72.233 | 1.00 | 44.37 | B |
| ATOM | 2183 | CG1 | VAL | 309 | 21.876 | −0.067 | 71.427 | 1.00 | 43.69 | B |
| ATOM | 2184 | CG2 | VAL | 309 | 20.076 | −1.737 | 71.912 | 1.00 | 43.50 | B |
| ATOM | 2185 | C | VAL | 309 | 21.188 | 1.234 | 74.075 | 1.00 | 41.50 | B |
| ATOM | 2186 | O | VAL | 309 | 20.368 | 2.091 | 73.788 | 1.00 | 41.50 | B |
| ATOM | 2187 | N | PRO | 310 | 22.351 | 1.535 | 74.675 | 1.00 | 38.54 | B |
| ATOM | 2188 | CD | PRO | 310 | 23.440 | 0.586 | 74.968 | 1.00 | 37.32 | B |
| ATOM | 2189 | CA | PRO | 310 | 22.736 | 2.898 | 75.058 | 1.00 | 37.55 | B |
| ATOM | 2190 | CB | PRO | 310 | 23.983 | 2.669 | 75.909 | 1.00 | 36.77 | B |
| ATOM | 2191 | CG | PRO | 310 | 24.614 | 1.502 | 75.238 | 1.00 | 36.14 | B |
| ATOM | 2192 | C | PRO | 310 | 22.977 | 3.898 | 73.917 | 1.00 | 36.95 | B |
| ATOM | 2193 | O | PRO | 310 | 24.042 | 4.493 | 73.827 | 1.00 | 36.57 | B |
| ATOM | 2194 | N | TYR | 311 | 21.972 | 4.076 | 73.061 | 1.00 | 36.05 | B |
| ATOM | 2195 | CA | TYR | 311 | 22.047 | 5.012 | 71.940 | 1.00 | 34.95 | B |
| ATOM | 2196 | CB | TYR | 311 | 20.778 | 4.949 | 71.085 | 1.00 | 35.41 | B |
| ATOM | 2197 | CG | TYR | 311 | 20.603 | 3.711 | 70.245 | 1.00 | 36.70 | B |
| ATOM | 2198 | CD1 | TYR | 311 | 21.603 | 3.289 | 69.374 | 1.00 | 35.89 | B |
| ATOM | 2199 | CE1 | TYR | 311 | 21.433 | 2.161 | 68.578 | 1.00 | 36.91 | B |
| ATOM | 2200 | CD2 | TYR | 311 | 19.416 | 2.973 | 70.300 | 1.00 | 36.75 | B |
| ATOM | 2201 | CE2 | TYR | 311 | 19.234 | 1.844 | 69.508 | 1.00 | 36.61 | B |
| ATOM | 2202 | CZ | TYR | 311 | 20.247 | 1.442 | 68.651 | 1.00 | 36.85 | B |
| ATOM | 2203 | OH | TYR | 311 | 20.086 | 0.312 | 67.882 | 1.00 | 35.56 | B |
| ATOM | 2204 | C | TYR | 311 | 22.217 | 6.462 | 72.402 | 1.00 | 35.12 | B |
| ATOM | 2205 | O | TYR | 311 | 23.038 | 7.186 | 71.868 | 1.00 | 34.13 | B |
| ATOM | 2206 | N | ARG | 312 | 21.422 | 6.868 | 73.392 | 1.00 | 34.48 | B |
| ATOM | 2207 | CA | ARG | 312 | 21.444 | 8.237 | 73.906 | 1.00 | 34.28 | B |
| ATOM | 2208 | CB | ARG | 312 | 20.160 | 8.523 | 74.690 | 1.00 | 35.83 | B |
| ATOM | 2209 | CG | ARG | 312 | 18.882 | 8.227 | 73.935 | 1.00 | 41.17 | B |
| ATOM | 2210 | CD | ARG | 312 | 17.732 | 8.007 | 74.897 | 1.00 | 44.62 | B |
| ATOM | 2211 | NE | ARG | 312 | 16.596 | 7.341 | 74.263 | 1.00 | 48.42 | B |
| ATOM | 2212 | CZ | ARG | 312 | 15.608 | 6.747 | 74.926 | 1.00 | 51.08 | B |
| ATOM | 2213 | NH1 | ARG | 312 | 15.610 | 6.732 | 76.254 | 1.00 | 50.32 | B |
| ATOM | 2214 | NH2 | ARG | 312 | 14.618 | 6.163 | 74.259 | 1.00 | 51.58 | B |
| ATOM | 2215 | C | ARG | 312 | 22.638 | 8.593 | 74.787 | 1.00 | 33.03 | B |
| ATOM | 2216 | O | ARG | 312 | 22.701 | 9.699 | 75.317 | 1.00 | 34.26 | B |
| ATOM | 2217 | N | GLU | 313 | 23.581 | 7.669 | 74.953 | 1.00 | 29.69 | B |
| ATOM | 2218 | CA | GLU | 313 | 24.735 | 7.947 | 75.799 | 1.00 | 25.30 | B |
| ATOM | 2219 | CB | GLU | 313 | 25.200 | 6.655 | 76.481 | 1.00 | 24.49 | B |
| ATOM | 2220 | CG | GLU | 313 | 24.278 | 6.242 | 77.634 | 1.00 | 25.08 | B |
| ATOM | 2221 | CD | GLU | 313 | 24.677 | 4.946 | 78.327 | 1.00 | 23.59 | B |
| ATOM | 2222 | OE1 | GLU | 313 | 25.883 | 4.722 | 78.553 | 1.00 | 23.79 | B |
| ATOM | 2223 | OE2 | GLU | 313 | 23.775 | 4.156 | 78.665 | 1.00 | 23.87 | B |
| ATOM | 2224 | C | GLU | 313 | 25.898 | 8.646 | 75.089 | 1.00 | 23.89 | B |
| ATOM | 2225 | O | GLU | 313 | 26.963 | 8.806 | 75.659 | 1.00 | 23.12 | B |
| ATOM | 2226 | N | SER | 314 | 25.680 | 9.068 | 73.843 | 1.00 | 21.70 | B |
| ATOM | 2227 | CA | SER | 314 | 26.714 | 9.766 | 73.080 | 1.00 | 21.61 | B |
| ATOM | 2228 | CB | SER | 314 | 27.800 | 8.796 | 72.622 | 1.00 | 19.78 | B |
| ATOM | 2229 | OG | SER | 314 | 27.401 | 8.118 | 71.442 | 1.00 | 17.85 | B |
| ATOM | 2230 | C | SER | 314 | 26.124 | 10.466 | 71.861 | 1.00 | 23.50 | B |
| ATOM | 2231 | O | SER | 314 | 25.047 | 10.105 | 71.388 | 1.00 | 23.43 | B |
| ATOM | 2232 | N | LYS | 315 | 26.840 | 11.462 | 71.348 | 1.00 | 23.77 | B |
| ATOM | 2233 | CA | LYS | 315 | 26.367 | 12.204 | 70.186 | 1.00 | 24.56 | B |
| ATOM | 2234 | CB | LYS | 315 | 27.216 | 13.462 | 69.963 | 1.00 | 24.98 | B |
| ATOM | 2235 | CG | LYS | 315 | 27.295 | 14.394 | 71.165 | 1.00 | 25.63 | B |
| ATOM | 2236 | CD | LYS | 315 | 25.926 | 14.862 | 71.607 | 1.00 | 25.73 | B |
| ATOM | 2237 | CE | LYS | 315 | 26.034 | 15.834 | 72.774 | 1.00 | 26.31 | B |
| ATOM | 2238 | NZ | LYS | 315 | 26.660 | 17.123 | 72.353 | 1.00 | 30.29 | B |
| ATOM | 2239 | C | LYS | 315 | 26.416 | 11.335 | 68.939 | 1.00 | 24.22 | B |
| ATOM | 2240 | O | LYS | 315 | 25.498 | 11.338 | 68.138 | 1.00 | 25.98 | B |
| ATOM | 2241 | N | LEU | 316 | 27.503 | 10.591 | 68.787 | 1.00 | 23.22 | B |
| ATOM | 2242 | CA | LEU | 316 | 27.674 | 9.719 | 67.636 | 1.00 | 24.18 | B |
| ATOM | 2243 | CB | LEU | 316 | 29.039 | 9.022 | 67.711 | 1.00 | 24.13 | B |
| ATOM | 2244 | CG | LEU | 316 | 29.451 | 8.205 | 66.488 | 1.00 | 23.55 | B |
| ATOM | 2245 | CD1 | LEU | 316 | 29.850 | 9.149 | 65.370 | 1.00 | 25.34 | B |
| ATOM | 2246 | CD2 | LEU | 316 | 30.609 | 7.299 | 66.840 | 1.00 | 22.84 | B |
| ATOM | 2247 | C | LEU | 316 | 26.567 | 8.664 | 67.506 | 1.00 | 23.18 | B |
| ATOM | 2248 | O | LEU | 316 | 25.892 | 8.590 | 66.480 | 1.00 | 22.77 | B |
| ATOM | 2249 | N | THR | 317 | 26.369 | 7.855 | 68.543 | 1.00 | 22.09 | B |
| ATOM | 2250 | CA | THR | 317 | 25.346 | 6.817 | 68.470 | 1.00 | 22.50 | B |
| ATOM | 2251 | CB | THR | 317 | 25.459 | 5.809 | 69.651 | 1.00 | 20.87 | B |
| ATOM | 2252 | OG1 | THR | 317 | 25.198 | 6.472 | 70.892 | 1.00 | 19.26 | B |
| ATOM | 2253 | CG2 | THR | 317 | 26.848 | 5.192 | 69.682 | 1.00 | 20.16 | B |
| ATOM | 2254 | C | THR | 317 | 23.923 | 7.367 | 68.394 | 1.00 | 23.49 | B |
| ATOM | 2255 | O | THR | 317 | 23.025 | 6.684 | 67.929 | 1.00 | 23.95 | B |
| ATOM | 2256 | N | ARG | 318 | 23.723 | 8.606 | 68.836 | 1.00 | 23.82 | B |
| ATOM | 2257 | CA | ARG | 318 | 22.402 | 9.225 | 68.764 | 1.00 | 25.01 | B |
| ATOM | 2258 | CB | ARG | 318 | 22.317 | 10.426 | 69.705 | 1.00 | 28.63 | B |
| ATOM | 2259 | CG | ARG | 318 | 21.923 | 10.065 | 71.120 | 1.00 | 34.53 | B |
| ATOM | 2260 | CD | ARG | 318 | 22.260 | 11.179 | 72.094 | 1.00 | 38.92 | B |
| ATOM | 2261 | NE | ARG | 318 | 21.606 | 12.436 | 71.745 | 1.00 | 45.13 | B |
| ATOM | 2262 | CZ | ARG | 318 | 20.293 | 12.642 | 71.792 | 1.00 | 47.64 | B |
| ATOM | 2263 | NH1 | ARG | 318 | 19.479 | 11.666 | 72.177 | 1.00 | 49.68 | B |
| ATOM | 2264 | NH2 | ARG | 318 | 19.796 | 13.826 | 71.456 | 1.00 | 45.41 | B |
| ATOM | 2265 | C | ARG | 318 | 22.127 | 9.674 | 67.335 | 1.00 | 24.81 | B |
| ATOM | 2266 | O | ARG | 318 | 21.015 | 9.522 | 66.828 | 1.00 | 24.93 | B |
| ATOM | 2267 | N | ILE | 319 | 23.149 | 10.217 | 66.684 | 1.00 | 22.86 | B |
| ATOM | 2268 | CA | ILE | 319 | 23.001 | 10.688 | 65.313 | 1.00 | 23.60 | B |
| ATOM | 2269 | CB | ILE | 319 | 24.197 | 11.588 | 64.893 | 1.00 | 22.37 | B |
| ATOM | 2270 | CG2 | ILE | 319 | 24.089 | 11.947 | 63.410 | 1.00 | 22.84 | B |
| ATOM | 2271 | CG1 | ILE | 319 | 24.224 | 12.861 | 65.748 | 1.00 | 22.76 | B |
| ATOM | 2272 | CD1 | ILE | 319 | 25.457 | 13.738 | 65.533 | 1.00 | 17.34 | B |
| ATOM | 2273 | C | ILE | 319 | 22.903 | 9.532 | 64.322 | 1.00 | 24.40 | B |
| ATOM | 2274 | O | ILE | 319 | 22.144 | 9.585 | 63.381 | 1.00 | 23.60 | B |
| ATOM | 2275 | N | LEU | 320 | 23.688 | 8.486 | 64.556 | 1.00 | 27.00 | B |
| ATOM | 2276 | CA | LEU | 320 | 23.725 | 7.331 | 63.664 | 1.00 | 28.83 | B |
| ATOM | 2277 | CB | LEU | 320 | 25.180 | 7.037 | 63.274 | 1.00 | 26.75 | B |
| ATOM | 2278 | CG | LEU | 320 | 26.035 | 8.151 | 62.668 | 1.00 | 28.19 | B |
| ATOM | 2279 | CD1 | LEU | 320 | 27.479 | 7.720 | 62.710 | 1.00 | 27.81 | B |
| ATOM | 2280 | CD2 | LEU | 320 | 25.601 | 8.459 | 61.237 | 1.00 | 26.81 | B |
| ATOM | 2281 | C | LEU | 320 | 23.098 | 6.053 | 64.220 | 1.00 | 30.42 | B |
| ATOM | 2282 | O | LEU | 320 | 23.501 | 4.957 | 63.841 | 1.00 | 31.06 | B |
| ATOM | 2283 | N | GLN | 321 | 22.097 | 6.188 | 65.085 | 1.00 | 32.73 | B |
| ATOM | 2284 | CA | GLN | 321 | 21.457 | 5.012 | 65.674 | 1.00 | 34.42 | B |
| ATOM | 2285 | CB | GLN | 321 | 20.466 | 5.419 | 66.777 | 1.00 | 35.23 | B |
| ATOM | 2286 | CG | GLN | 321 | 19.195 | 6.116 | 66.314 | 1.00 | 39.71 | B |
| ATOM | 2287 | CD | GLN | 321 | 18.320 | 6.569 | 67.488 | 1.00 | 42.32 | B |
| ATOM | 2288 | OE1 | GLN | 321 | 17.881 | 5.755 | 68.298 | 1.00 | 42.09 | B |
| ATOM | 2289 | NE2 | GLN | 321 | 18.069 | 7.877 | 67.577 | 1.00 | 44.14 | B |
| ATOM | 2290 | C | GLN | 321 | 20.758 | 4.102 | 64.663 | 1.00 | 33.44 | B |
| ATOM | 2291 | O | GLN | 321 | 20.677 | 2.901 | 64.868 | 1.00 | 34.48 | B |
| ATOM | 2292 | N | ASP | 322 | 20.261 | 4.666 | 63.569 | 1.00 | 32.24 | B |
| ATOM | 2293 | CA | ASP | 322 | 19.583 | 3.839 | 62.575 | 1.00 | 33.02 | B |
| ATOM | 2294 | CB | ASP | 322 | 18.780 | 4.693 | 61.595 | 1.00 | 32.22 | B |
| ATOM | 2295 | CG | ASP | 322 | 17.790 | 3.871 | 60.783 | 1.00 | 32.38 | B |
| ATOM | 2296 | OD1 | ASP | 322 | 17.716 | 4.061 | 59.548 | 1.00 | 32.08 | B |
| ATOM | 2297 | OD2 | ASP | 322 | 17.074 | 3.045 | 61.382 | 1.00 | 30.54 | B |
| ATOM | 2298 | C | ASP | 322 | 20.598 | 3.011 | 61.794 | 1.00 | 32.49 | B |
| ATOM | 2299 | O | ASP | 322 | 20.228 | 2.175 | 60.988 | 1.00 | 32.45 | B |
| ATOM | 2300 | N | SER | 323 | 21.880 | 3.274 | 62.030 | 1.00 | 32.77 | B |
| ATOM | 2301 | CA | SER | 323 | 22.951 | 2.547 | 61.361 | 1.00 | 30.97 | B |
| ATOM | 2302 | CB | SER | 323 | 24.122 | 3.480 | 61.067 | 1.00 | 28.95 | B |
| ATOM | 2303 | OG | SER | 323 | 23.837 | 4.320 | 59.959 | 1.00 | 27.41 | B |
| ATOM | 2304 | C | SER | 323 | 23.416 | 1.374 | 62.224 | 1.00 | 30.75 | B |
| ATOM | 2305 | O | SER | 323 | 24.171 | 0.517 | 61.783 | 1.00 | 29.17 | B |
| ATOM | 2306 | N | LEU | 324 | 22.966 | 1.352 | 63.470 | 1.00 | 30.45 | B |
| ATOM | 2307 | CA | LEU | 324 | 23.326 | 0.270 | 64.363 | 1.00 | 31.28 | B |
| ATOM | 2308 | CB | LEU | 324 | 24.046 | 0.809 | 65.606 | 1.00 | 31.28 | B |
| ATOM | 2309 | CG | LEU | 324 | 25.476 | 1.353 | 65.463 | 1.00 | 32.14 | B |
| ATOM | 2310 | CD1 | LEU | 324 | 26.308 | 0.424 | 64.587 | 1.00 | 33.04 | B |
| ATOM | 2311 | CD2 | LEU | 324 | 25.436 | 2.739 | 64.862 | 1.00 | 34.26 | B |
| ATOM | 2312 | C | LEU | 324 | 22.081 | −0.511 | 64.771 | 1.00 | 31.54 | B |
| ATOM | 2313 | O | LEU | 324 | 21.468 | −0.235 | 65.785 | 1.00 | 31.30 | B |
| ATOM | 2314 | N | GLY | 325 | 21.715 | −1.490 | 63.950 | 1.00 | 33.73 | B |
| ATOM | 2315 | CA | GLY | 325 | 20.554 | −2.311 | 64.249 | 1.00 | 33.79 | B |
| ATOM | 2316 | C | GLY | 325 | 19.244 | −1.636 | 63.901 | 1.00 | 33.20 | B |
| ATOM | 2317 | O | GLY | 325 | 18.218 | −1.905 | 64.517 | 1.00 | 33.16 | B |
| ATOM | 2318 | N | GLY | 326 | 19.286 | −0.754 | 62.909 | 1.00 | 32.43 | B |
| ATOM | 2319 | CA | GLY | 326 | 18.090 | −0.048 | 62.499 | 1.00 | 33.13 | B |
| ATOM | 2320 | C | GLY | 326 | 17.704 | −0.420 | 61.088 | 1.00 | 34.86 | B |
| ATOM | 2321 | O | GLY | 326 | 17.905 | −1.541 | 60.680 | 1.00 | 34.93 | B |
| ATOM | 2322 | N | ARG | 327 | 17.157 | 0.535 | 60.343 | 1.00 | 37.13 | B |
| ATOM | 2323 | CA | ARG | 327 | 16.748 | 0.278 | 58.974 | 1.00 | 38.94 | B |
| ATOM | 2324 | CB | ARG | 327 | 15.327 | 0.784 | 58.753 | 1.00 | 43.05 | B |
| ATOM | 2325 | CG | ARG | 327 | 14.278 | 0.034 | 59.559 | 1.00 | 49.59 | B |
| ATOM | 2326 | CD | ARG | 327 | 12.872 | 0.464 | 59.159 | 1.00 | 54.64 | B |
| ATOM | 2327 | NE | ARG | 327 | 12.071 | −0.657 | 58.665 | 1.00 | 60.40 | B |
| ATOM | 2328 | CZ | ARG | 327 | 12.358 | −1.380 | 57.583 | 1.00 | 62.77 | B |
| ATOM | 2329 | NH1 | ARG | 327 | 13.441 | −1.105 | 56.861 | 1.00 | 63.46 | B |
| ATOM | 2330 | NH2 | ARG | 327 | 11.556 | −2.377 | 57.219 | 1.00 | 61.73 | B |
| ATOM | 2331 | C | ARG | 327 | 17.686 | 0.887 | 57.934 | 1.00 | 38.03 | B |
| ATOM | 2332 | O | ARG | 327 | 17.249 | 1.289 | 56.869 | 1.00 | 37.61 | B |
| ATOM | 2333 | N | THR | 328 | 18.979 | 0.931 | 58.252 | 1.00 | 36.37 | B |
| ATOM | 2334 | CA | THR | 328 | 19.983 | 1.481 | 57.345 | 1.00 | 35.54 | B |
| ATOM | 2335 | CB | THR | 328 | 20.715 | 2.685 | 57.989 | 1.00 | 34.89 | B |
| ATOM | 2336 | OG1 | THR | 328 | 19.798 | 3.762 | 56.194 | 1.00 | 35.66 | B |
| ATOM | 2337 | CG2 | THR | 328 | 21.847 | 3.156 | 57.096 | 1.00 | 33.72 | B |
| ATOM | 2338 | C | THR | 328 | 21.040 | 0.442 | 56.974 | 1.00 | 34.98 | B |
| ATOM | 2339 | O | THR | 328 | 21.630 | −0.170 | 57.848 | 1.00 | 36.65 | B |
| ATOM | 2340 | N | ARG | 329 | 21.274 | 0.252 | 55.678 | 1.00 | 33.43 | B |
| ATOM | 2341 | CA | ARG | 329 | 22.281 | −0.704 | 55.226 | 1.00 | 33.67 | B |
| ATOM | 2342 | CB | ARG | 329 | 22.354 | −0.752 | 53.696 | 1.00 | 35.61 | B |
| ATOM | 2343 | CG | ARG | 329 | 23.146 | −1.938 | 53.156 | 1.00 | 40.29 | B |
| ATOM | 2344 | CD | ARG | 329 | 23.642 | −1.691 | 51.736 | 1.00 | 45.76 | B |
| ATOM | 2345 | NE | ARG | 329 | 24.253 | −2.877 | 51.133 | 1.00 | 51.83 | B |
| ATOM | 2346 | CZ | ARG | 329 | 25.297 | −3.540 | 51.632 | 1.00 | 54.83 | B |
| ATOM | 2347 | NH1 | ARG | 329 | 25.874 | −3.148 | 52.761 | 1.00 | 54.64 | B |
| ATOM | 2348 | NH2 | ARG | 329 | 25.772 | −4.601 | 50.991 | 1.00 | 56.00 | B |
| ATOM | 2349 | C | ARG | 329 | 23.615 | −0.218 | 55.764 | 1.00 | 30.92 | B |
| ATOM | 2350 | O | ARG | 329 | 24.034 | 0.871 | 55.452 | 1.00 | 33.46 | B |
| ATOM | 2351 | N | THR | 330 | 24.277 | −1.028 | 56.573 | 1.00 | 28.10 | B |
| ATOM | 2352 | CA | THR | 330 | 25.541 | −0.622 | 57.156 | 1.00 | 26.64 | B |
| ATOM | 2353 | CB | THR | 330 | 25.410 | −0.524 | 58.691 | 1.00 | 25.12 | B |
| ATOM | 2354 | OG1 | THR | 330 | 24.526 | 0.549 | 59.019 | 1.00 | 25.09 | B |
| ATOM | 2355 | CG2 | THR | 330 | 26.760 | −0.291 | 59.351 | 1.00 | 22.76 | B |
| ATOM | 2356 | C | THR | 330 | 26.723 | −1.516 | 56.820 | 1.00 | 27.27 | B |
| ATOM | 2357 | O | THR | 330 | 26.602 | −2.732 | 56.748 | 1.00 | 27.57 | B |
| ATOM | 2358 | N | SER | 331 | 27.868 | −0.878 | 56.618 | 1.00 | 26.82 | B |
| ATOM | 2359 | CA | SER | 331 | 29.104 | −1.567 | 56.308 | 1.00 | 26.67 | B |
| ATOM | 2360 | CB | SER | 331 | 29.442 | −1.446 | 54.830 | 1.00 | 26.29 | B |
| ATOM | 2361 | OG | SER | 331 | 28.444 | −2.072 | 54.052 | 1.00 | 31.25 | B |
| ATOM | 2362 | C | SER | 331 | 30.191 | −0.907 | 57.125 | 1.00 | 26.05 | B |
| ATOM | 2363 | O | SER | 331 | 30.210 | 0.304 | 57.272 | 1.00 | 29.07 | B |
| ATOM | 2364 | N | ILE | 332 | 31.086 | −1.712 | 57.677 | 1.00 | 24.35 | B |
| ATOM | 2365 | CA | ILE | 332 | 32.179 | −1.190 | 58.472 | 1.00 | 20.58 | B |
| ATOM | 2366 | CB | ILE | 332 | 32.119 | −1.704 | 59.917 | 1.00 | 16.78 | B |
| ATOM | 2367 | CG2 | ILE | 332 | 33.367 | −1.290 | 60.656 | 1.00 | 15.30 | B |
| ATOM | 2368 | CG1 | ILE | 332 | 30.849 | −1.195 | 60.605 | 1.00 | 14.73 | B |
| ATOM | 2369 | CD1 | ILE | 332 | 30.641 | −1.735 | 62.018 | 1.00 | 11.20 | B |
| ATOM | 2370 | C | ILE | 332 | 33.484 | −1.646 | 57.855 | 1.00 | 22.60 | B |
| ATOM | 2371 | O | ILE | 332 | 33.635 | −2.809 | 57.495 | 1.00 | 22.21 | B |
| ATOM | 2372 | N | ILE | 333 | 34.421 | −0.718 | 57.713 | 1.00 | 23.08 | B |
| ATOM | 2373 | CA | ILE | 333 | 35.718 | −1.046 | 57.148 | 1.00 | 21.26 | B |
| ATOM | 2374 | CB | ILE | 333 | 36.096 | −0.086 | 56.011 | 1.00 | 20.77 | B |
| ATOM | 2375 | CG2 | ILE | 333 | 37.401 | −0.530 | 55.375 | 1.00 | 20.19 | B |
| ATOM | 2376 | CG1 | ILE | 333 | 34.993 | −0.065 | 54.950 | 1.00 | 22.76 | B |
| ATOM | 2377 | CD1 | ILE | 333 | 35.297 | 0.826 | 53.738 | 1.00 | 19.77 | B |
| ATOM | 2378 | C | ILE | 333 | 36.736 | −0.927 | 58.267 | 1.00 | 22.44 | B |
| ATOM | 2379 | O | ILE | 333 | 37.015 | 0.170 | 58.740 | 1.00 | 25.05 | B |
| ATOM | 2380 | N | ALA | 334 | 37.269 | −2.061 | 58.708 | 1.00 | 22.25 | B |
| ATOM | 2381 | CA | ALA | 334 | 38.252 | −2.080 | 59.783 | 1.00 | 21.24 | B |
| ATOM | 2382 | CB | ALA | 334 | 38.088 | −3.351 | 60.605 | 1.00 | 21.16 | B |
| ATOM | 2383 | C | ALA | 334 | 39.667 | −1.998 | 59.212 | 1.00 | 20.54 | B |
| ATOM | 2384 | O | ALA | 334 | 40.070 | −2.850 | 58.452 | 1.00 | 21.75 | B |
| ATOM | 2385 | N | THR | 335 | 40.405 | −0.952 | 59.582 | 1.00 | 18.02 | B |
| ATOM | 2386 | CA | THR | 335 | 41.772 | −0.771 | 59.102 | 1.00 | 15.52 | B |
| ATOM | 2387 | CB | THR | 335 | 42.052 | 0.701 | 58.752 | 1.00 | 14.93 | B |
| ATOM | 2388 | OG1 | THR | 335 | 41.551 | 1.558 | 59.794 | 1.00 | 16.56 | B |
| ATOM | 2389 | CG2 | THR | 335 | 41.394 | 1.051 | 57.447 | 1.00 | 13.76 | B |
| ATOM | 2390 | C | THR | 335 | 42.780 | −1.257 | 60.132 | 1.00 | 14.40 | B |
| ATOM | 2391 | O | THR | 335 | 42.586 | −1.096 | 61.340 | 1.00 | 13.68 | B |
| ATOM | 2392 | N | ILE | 336 | 43.863 | −1.849 | 59.641 | 1.00 | 15.75 | B |
| ATOM | 2393 | CA | ILE | 336 | 44.893 | −2.409 | 60.506 | 1.00 | 16.07 | B |
| ATOM | 2394 | CB | ILE | 336 | 44.671 | −3.936 | 60.702 | 1.00 | 14.75 | B |
| ATOM | 2395 | CG2 | ILE | 336 | 43.346 | −4.185 | 61.401 | 1.00 | 13.27 | B |
| ATOM | 2396 | CG1 | ILE | 336 | 44.678 | −4.662 | 59.348 | 1.00 | 15.22 | B |
| ATOM | 2397 | CD1 | ILE | 336 | 44.726 | −6.215 | 59.461 | 1.00 | 13.20 | B |
| ATOM | 2398 | C | ILE | 336 | 46.317 | −2.186 | 59.999 | 1.00 | 17.99 | B |
| ATOM | 2399 | O | ILE | 336 | 46.534 | −1.816 | 58.844 | 1.00 | 17.06 | B |
| ATOM | 2400 | N | SER | 337 | 47.280 | −2.407 | 60.889 | 1.00 | 20.83 | B |
| ATOM | 2401 | CA | SER | 337 | 48.694 | −2.250 | 60.570 | 1.00 | 23.58 | B |
| ATOM | 2402 | CB | SER | 337 | 49.399 | −1.491 | 61.685 | 1.00 | 22.57 | B |
| ATOM | 2403 | OG | SER | 337 | 50.792 | −1.737 | 61.645 | 1.00 | 21.86 | B |
| ATOM | 2404 | C | SER | 337 | 49.395 | −3.600 | 60.389 | 1.00 | 27.32 | B |
| ATOM | 2405 | O | SER | 337 | 49.123 | −4.548 | 61.122 | 1.00 | 27.36 | B |
| ATOM | 2406 | N | PRO | 338 | 50.320 | −3.688 | 59.416 | 1.00 | 28.03 | B |
| ATOM | 2407 | CD | PRO | 338 | 50.612 | −2.678 | 58.383 | 1.00 | 29.38 | B |
| ATOM | 2408 | CA | PRO | 338 | 51.063 | −4.919 | 59.147 | 1.00 | 30.56 | B |
| ATOM | 2409 | CB | PRO | 338 | 51.485 | −4.743 | 57.698 | 1.00 | 29.47 | B |
| ATOM | 2410 | CG | PRO | 338 | 51.804 | −3.283 | 57.657 | 1.00 | 28.25 | B |
| ATOM | 2411 | C | PRO | 338 | 52.274 | −5.047 | 60.074 | 1.00 | 31.99 | B |
| ATOM | 2412 | O | PRO | 338 | 52.903 | −6.083 | 60.131 | 1.00 | 32.55 | B |
| ATOM | 2413 | N | ALA | 339 | 52.586 | −3.972 | 60.790 | 1.00 | 33.15 | B |
| ATOM | 2414 | CA | ALA | 339 | 53.732 | −3.955 | 61.690 | 1.00 | 34.44 | B |
| ATOM | 2415 | CB | ALA | 339 | 54.051 | −2.518 | 62.109 | 1.00 | 35.58 | B |
| ATOM | 2416 | C | ALA | 339 | 53.505 | −4.816 | 62.918 | 1.00 | 35.05 | B |
| ATOM | 2417 | O | ALA | 339 | 52.391 | −4.956 | 63.386 | 1.00 | 35.58 | B |
| ATOM | 2418 | N | SER | 340 | 54.585 | −5.380 | 63.447 | 1.00 | 36.34 | B |
| ATOM | 2419 | CA | SER | 340 | 54.479 | −6.236 | 64.615 | 1.00 | 36.42 | B |
| ATOM | 2420 | CB | SER | 340 | 55.694 | −7.162 | 64.717 | 1.00 | 36.55 | B |
| ATOM | 2421 | OG | SER | 340 | 56.891 | −6.431 | 64.909 | 1.00 | 37.23 | B |
| ATOM | 2422 | C | SER | 340 | 54.324 | −5.457 | 65.914 | 1.00 | 36.18 | B |
| ATOM | 2423 | O | SER | 340 | 53.769 | −5.969 | 66.871 | 1.00 | 36.17 | B |
| ATOM | 2424 | N | LEU | 341 | 54.803 | −4.220 | 65.957 | 1.00 | 36.13 | B |
| ATOM | 2425 | CA | LEU | 341 | 54.664 | −3.453 | 67.190 | 1.00 | 38.21 | B |
| ATOM | 2426 | CB | LEU | 341 | 55.663 | −2.296 | 67.239 | 1.00 | 40.75 | B |
| ATOM | 2427 | CG | LEU | 341 | 55.293 | −1.011 | 66.500 | 1.00 | 44.27 | B |
| ATOM | 2428 | CD1 | LEU | 341 | 56.054 | 0.160 | 67.121 | 1.00 | 44.94 | B |
| ATOM | 2429 | CD2 | LEU | 341 | 55.597 | −1.158 | 65.011 | 1.00 | 45.97 | B |
| ATOM | 2430 | C | LEU | 341 | 53.244 | −2.912 | 67.337 | 1.00 | 36.82 | B |
| ATOM | 2431 | O | LEU | 341 | 52.944 | −2.185 | 68.259 | 1.00 | 37.65 | B |
| ATOM | 2432 | N | ASN | 342 | 52.376 | −3.288 | 66.408 | 1.00 | 36.59 | B |
| ATOM | 2433 | CA | ASN | 342 | 50.983 | −2.856 | 66.416 | 1.00 | 35.71 | B |
| ATOM | 2434 | CB | ASN | 342 | 50.636 | −2.219 | 65.071 | 1.00 | 34.64 | B |
| ATOM | 2435 | CG | ASN | 342 | 51.343 | −0.903 | 64.865 | 1.00 | 34.11 | B |
| ATOM | 2436 | OD1 | ASN | 342 | 51.904 | −0.649 | 63.808 | 1.00 | 32.85 | B |
| ATOM | 2437 | ND2 | ASN | 342 | 51.315 | −0.052 | 65.888 | 1.00 | 32.94 | B |
| ATOM | 2438 | C | ASN | 342 | 50.084 | −4.048 | 66.661 | 1.00 | 35.91 | B |
| ATOM | 2439 | O | ASN | 342 | 48.860 | −3.958 | 66.561 | 1.00 | 37.26 | B |
| ATOM | 2440 | N | LEU | 343 | 50.720 | −5.164 | 66.993 | 1.00 | 34.56 | B |
| ATOM | 2441 | CA | LEU | 343 | 50.033 | −6.419 | 67.244 | 1.00 | 32.49 | B |
| ATOM | 2442 | CB | LEU | 343 | 51.019 | −7.433 | 67.836 | 1.00 | 31.23 | B |
| ATOM | 2443 | CG | LEU | 343 | 50.546 | −8.858 | 68.135 | 1.00 | 31.25 | B |
| ATOM | 2444 | CD1 | LEU | 343 | 50.001 | −8.944 | 69.548 | 1.00 | 32.82 | B |
| ATOM | 2445 | CD2 | LEU | 343 | 49.504 | −9.286 | 67.101 | 1.00 | 30.64 | B |
| ATOM | 2446 | C | LEU | 343 | 48.817 | −6.295 | 68.140 | 1.00 | 30.37 | B |
| ATOM | 2447 | O | LEU | 343 | 47.714 | −6.608 | 67.732 | 1.00 | 29.24 | B |
| ATOM | 2448 | N | GLU | 344 | 49.023 | −5.831 | 69.364 | 1.00 | 30.64 | B |
| ATOM | 2449 | CA | GLU | 344 | 47.922 | −5.710 | 70.307 | 1.00 | 32.19 | B |
| ATOM | 2450 | CB | GLU | 344 | 48.442 | −5.121 | 71.619 | 1.00 | 34.78 | B |
| ATOM | 2451 | CG | GLU | 344 | 47.460 | −5.189 | 72.761 | 1.00 | 42.18 | B |
| ATOM | 2452 | CD | GLU | 344 | 48.107 | −4.861 | 74.099 | 1.00 | 47.80 | B |
| ATOM | 2453 | OE1 | GLU | 344 | 48.743 | −3.785 | 74.209 | 1.00 | 48.41 | B |
| ATOM | 2454 | OE2 | GLU | 344 | 47.982 | −5.686 | 75.036 | 1.00 | 49.00 | B |
| ATOM | 2455 | C | GLU | 344 | 46.736 | −4.899 | 69.760 | 1.00 | 30.46 | B |
| ATOM | 2456 | O | GLU | 344 | 45.600 | −5.355 | 69.802 | 1.00 | 29.53 | B |
| ATOM | 2457 | N | GLU | 345 | 46.991 | −3.707 | 69.234 | 1.00 | 29.30 | B |
| ATOM | 2458 | CA | GLU | 345 | 45.901 | −2.891 | 68.703 | 1.00 | 29.30 | B |
| ATOM | 2459 | CB | GLU | 345 | 46.393 | −1.477 | 68.349 | 1.00 | 29.27 | B |
| ATOM | 2460 | CG | GLU | 345 | 46.618 | −0.581 | 69.565 | 1.00 | 29.72 | B |
| ATOM | 2461 | CD | GLU | 345 | 45.337 | −0.285 | 70.330 | 1.00 | 30.47 | B |
| ATOM | 2462 | OE1 | GLU | 345 | 45.429 | 0.193 | 71.482 | 1.00 | 33.09 | B |
| ATOM | 2463 | OE2 | GLU | 345 | 44.241 | −0.521 | 69.786 | 1.00 | 30.71 | B |
| ATOM | 2464 | C | GLU | 345 | 45.277 | −3.556 | 67.476 | 1.00 | 27.38 | B |
| ATOM | 2465 | O | GLU | 345 | 44.082 | −3.423 | 67.233 | 1.00 | 28.53 | B |
| ATOM | 2466 | N | THR | 346 | 46.084 | −4.283 | 66.711 | 1.00 | 24.59 | B |
| ATOM | 2467 | CA | THR | 346 | 45.576 | −4.979 | 65.530 | 1.00 | 23.55 | B |
| ATOM | 2468 | CB | THR | 346 | 46.717 | −5.588 | 64.721 | 1.00 | 22.82 | B |
| ATOM | 2469 | OG1 | THR | 346 | 47.503 | −4.534 | 64.147 | 1.00 | 24.62 | B |
| ATOM | 2470 | CG2 | THR | 346 | 46.173 | −6.473 | 63.618 | 1.00 | 23.82 | B |
| ATOM | 2471 | C | THR | 346 | 44.597 | −6.083 | 65.937 | 1.00 | 22.61 | B |
| ATOM | 2472 | O | THR | 346 | 43.617 | −6.343 | 65.252 | 1.00 | 22.38 | B |
| ATOM | 2473 | N | LEU | 347 | 44.873 | −6.732 | 67.062 | 1.00 | 23.16 | B |
| ATOM | 2474 | CA | LEU | 347 | 44.002 | −7.790 | 67.561 | 1.00 | 23.19 | B |
| ATOM | 2475 | CB | LEU | 347 | 44.678 | −8.568 | 68.696 | 1.00 | 21.66 | B |
| ATOM | 2476 | CG | LEU | 347 | 45.955 | −9.346 | 68.374 | 1.00 | 22.14 | B |
| ATOM | 2477 | CD1 | LEU | 347 | 46.393 | −10.118 | 69.613 | 1.00 | 20.42 | B |
| ATOM | 2478 | CD2 | LEU | 347 | 45.718 | −10.293 | 67.210 | 1.00 | 22.20 | B |
| ATOM | 2479 | C | LEU | 347 | 42.679 | −7.203 | 68.063 | 1.00 | 23.83 | B |
| ATOM | 2480 | O | LEU | 347 | 41.617 | −7.712 | 67.732 | 1.00 | 25.14 | B |
| ATOM | 2481 | N | SER | 348 | 42.743 | −6.135 | 68.854 | 1.00 | 21.92 | B |
| ATOM | 2482 | CA | SER | 348 | 41.518 | −5.530 | 69.368 | 1.00 | 23.12 | B |
| ATOM | 2483 | CB | SER | 348 | 41.839 | −4.306 | 70.215 | 1.00 | 21.23 | B |
| ATOM | 2484 | OG | SER | 348 | 42.491 | −4.707 | 71.402 | 1.00 | 27.13 | B |
| ATOM | 2485 | C | SER | 348 | 40.582 | −5.144 | 68.238 | 1.00 | 22.86 | B |
| ATOM | 2486 | O | SER | 348 | 39.384 | −5.348 | 68.331 | 1.00 | 22.12 | B |
| ATOM | 2487 | N | THR | 349 | 41.156 | −4.596 | 67.172 | 1.00 | 23.05 | B |
| ATOM | 2488 | CA | THR | 349 | 40.391 | −4.186 | 66.005 | 1.00 | 25.38 | B |
| ATOM | 2489 | CB | THR | 349 | 41.309 | −3.483 | 64.988 | 1.00 | 25.69 | B |
| ATOM | 2490 | OG1 | THR | 349 | 41.656 | −2.185 | 65.495 | 1.00 | 28.94 | B |
| ATOM | 2491 | CG2 | THR | 349 | 40.627 | −3.334 | 63.639 | 1.00 | 26.37 | B |
| ATOM | 2492 | C | THR | 349 | 39.714 | −5.387 | 65.344 | 1.00 | 27.04 | B |
| ATOM | 2493 | O | THR | 349 | 38.502 | −5.396 | 65.164 | 1.00 | 25.10 | B |
| ATOM | 2494 | N | LEU | 350 | 40.505 | −6.399 | 64.988 | 1.00 | 29.73 | B |
| ATOM | 2495 | CA | LEU | 350 | 39.971 | −7.610 | 64.352 | 1.00 | 32.43 | B |
| ATOM | 2496 | CB | LEU | 350 | 41.112 | −8.602 | 64.087 | 1.00 | 32.67 | B |
| ATOM | 2497 | CG | LEU | 350 | 41.782 | −8.523 | 62.709 | 1.00 | 33.86 | B |
| ATOM | 2498 | CD1 | LEU | 350 | 41.867 | −7.089 | 62.243 | 1.00 | 35.72 | B |
| ATOM | 2499 | CD2 | LEU | 350 | 43.160 | −9.140 | 62.777 | 1.00 | 34.30 | B |
| ATOM | 2500 | C | LEU | 350 | 38.880 | −8.268 | 65.203 | 1.00 | 32.13 | B |
| ATOM | 2501 | O | LEU | 350 | 37.869 | −8.736 | 64.693 | 1.00 | 31.89 | B |
| ATOM | 2502 | N | GLU | 351 | 39.104 | −8.286 | 66.510 | 1.00 | 32.99 | B |
| ATOM | 2503 | CA | GLU | 351 | 38.163 | −8.869 | 67.452 | 1.00 | 33.24 | B |
| ATOM | 2504 | CB | GLU | 351 | 38.807 | −8.951 | 68.837 | 1.00 | 36.70 | B |
| ATOM | 2505 | CG | GLU | 351 | 38.014 | −9.772 | 69.821 | 1.00 | 44.06 | B |
| ATOM | 2506 | CD | GLU | 351 | 37.791 | −11.179 | 69.309 | 1.00 | 47.54 | B |
| ATOM | 2507 | OE1 | GLU | 351 | 38.805 | −11.848 | 68.982 | 1.00 | 48.67 | B |
| ATOM | 2508 | OE2 | GLU | 351 | 36.610 | −11.599 | 69.228 | 1.00 | 48.07 | B |
| ATOM | 2509 | C | GLU | 351 | 36.901 | −8.009 | 67.519 | 1.00 | 31.83 | B |
| ATOM | 2510 | O | GLU | 351 | 35.778 | −8.532 | 67.584 | 1.00 | 32.55 | B |
| ATOM | 2511 | N | TYR | 352 | 37.097 | −6.690 | 67.503 | 1.00 | 29.09 | B |
| ATOM | 2512 | CA | TYR | 352 | 35.997 | −5.727 | 67.550 | 1.00 | 25.10 | B |
| ATOM | 2513 | CB | TYR | 352 | 36.561 | −4.318 | 67.758 | 1.00 | 23.54 | B |
| ATOM | 2514 | CG | TYR | 352 | 35.537 | −3.220 | 67.970 | 1.00 | 23.52 | B |
| ATOM | 2515 | CD1 | TYR | 352 | 34.862 | −2.642 | 66.893 | 1.00 | 21.07 | B |
| ATOM | 2516 | CE1 | TYR | 352 | 33.952 | −1.601 | 67.086 | 1.00 | 22.50 | B |
| ATOM | 2517 | CD2 | TYR | 352 | 35.271 | −2.734 | 69.254 | 1.00 | 23.10 | B |
| ATOM | 2518 | CE2 | TYR | 352 | 34.366 | −1.699 | 69.464 | 1.00 | 22.61 | B |
| ATOM | 2519 | CZ | TYR | 352 | 33.712 | −1.134 | 68.377 | 1.00 | 25.05 | B |
| ATOM | 2520 | OH | TYR | 352 | 32.840 | −0.085 | 68.577 | 1.00 | 29.15 | B |
| ATOM | 2521 | C | TYR | 352 | 35.169 | −5.790 | 66.262 | 1.00 | 23.04 | B |
| ATOM | 2522 | O | TYR | 352 | 33.957 | −5.819 | 66.309 | 1.00 | 21.96 | B |
| ATOM | 2523 | N | ALA | 353 | 35.841 | −5.821 | 65.117 | 1.00 | 21.97 | B |
| ATOM | 2524 | CA | ALA | 353 | 35.155 | −5.883 | 63.826 | 1.00 | 24.73 | B |
| ATOM | 2525 | CB | ALA | 353 | 36.163 | −5.732 | 62.692 | 1.00 | 21.20 | B |
| ATOM | 2526 | C | ALA | 353 | 34.380 | −7.192 | 63.663 | 1.00 | 26.52 | B |
| ATOM | 2527 | O | ALA | 353 | 33.283 | −7.210 | 63.119 | 1.00 | 25.94 | B |
| ATOM | 2528 | N | HIS | 354 | 34.978 | −8.282 | 64.138 | 1.00 | 30.11 | B |
| ATOM | 2529 | CA | HIS | 354 | 34.375 | −9.607 | 64.052 | 1.00 | 32.42 | B |
| ATOM | 2530 | CB | HIS | 354 | 35.334 | −10.660 | 64.626 | 1.00 | 35.26 | B |
| ATOM | 2531 | CG | HIS | 354 | 34.939 | −12.073 | 64.317 | 1.00 | 38.11 | B |
| ATOM | 2532 | CD2 | HIS | 354 | 34.416 | −13.045 | 65.103 | 1.00 | 38.24 | B |
| ATOM | 2533 | ND1 | HIS | 354 | 35.045 | −12.614 | 63.053 | 1.00 | 39.29 | B |
| ATOM | 2534 | CE1 | HIS | 354 | 34.600 | −13.858 | 63.072 | 1.00 | 38.94 | B |
| ATOM | 2535 | NE2 | HIS | 354 | 34.213 | −14.143 | 64.303 | 1.00 | 39.79 | B |
| ATOM | 2536 | C | HIS | 354 | 33.050 | −9.642 | 64.811 | 1.00 | 33.09 | B |
| ATOM | 2537 | O | HIS | 354 | 32.048 | −10.127 | 64.297 | 1.00 | 33.51 | B |
| ATOM | 2538 | N | ARG | 355 | 33.053 | −9.122 | 66.034 | 1.00 | 33.22 | B |
| ATOM | 2539 | CA | ARG | 355 | 31.847 | −9.091 | 66.852 | 1.00 | 35.31 | B |
| ATOM | 2540 | CB | ARG | 355 | 32.145 | −8.470 | 68.220 | 1.00 | 38.27 | B |
| ATOM | 2541 | CG | ARG | 355 | 32.976 | −9.320 | 69.155 | 1.00 | 41.93 | B |
| ATOM | 2542 | CD | ARG | 355 | 33.322 | −8.539 | 70.416 | 1.00 | 44.68 | B |
| ATOM | 2543 | NE | ARG | 355 | 32.132 | −8.099 | 71.142 | 1.00 | 46.84 | B |
| ATOM | 2544 | CZ | ARG | 355 | 31.299 | −8.915 | 71.781 | 1.00 | 48.76 | B |
| ATOM | 2545 | NH1 | ARG | 355 | 31.523 | −10.222 | 71.785 | 1.00 | 48.40 | B |
| ATOM | 2546 | NH2 | ARG | 355 | 30.243 | −8.423 | 72.420 | 1.00 | 47.82 | B |
| ATOM | 2547 | C | ARG | 355 | 30.740 | −8.281 | 66.173 | 1.00 | 35.52 | B |
| ATOM | 2548 | O | ARG | 355 | 29.564 | −8.610 | 66.297 | 1.00 | 36.07 | B |
| ATOM | 2549 | N | ALA | 356 | 31.124 | −7.228 | 65.454 | 1.00 | 33.02 | B |
| ATOM | 2550 | CA | ALA | 356 | 30.146 | −6.374 | 64.789 | 1.00 | 31.19 | B |
| ATOM | 2551 | CB | ALA | 356 | 30.837 | −5.156 | 64.206 | 1.00 | 31.50 | B |
| ATOM | 2552 | C | ALA | 356 | 29.342 | −7.089 | 63.704 | 1.00 | 31.06 | B |
| ATOM | 2553 | O | ALA | 356 | 28.259 | −6.645 | 63.343 | 1.00 | 28.55 | B |
| ATOM | 2554 | N | LYS | 357 | 29.880 | −8.197 | 63.194 | 1.00 | 31.69 | B |
| ATOM | 2555 | CA | LYS | 357 | 29.215 | −8.973 | 62.144 | 1.00 | 33.26 | B |
| ATOM | 2556 | CB | LYS | 357 | 30.060 | −10.198 | 61.768 | 1.00 | 35.45 | B |
| ATOM | 2557 | CG | LYS | 357 | 31.491 | −9.906 | 61.350 | 1.00 | 36.26 | B |
| ATOM | 2558 | CD | LYS | 357 | 31.791 | −10.458 | 59.956 | 1.00 | 39.94 | B |
| ATOM | 2559 | CE | LYS | 357 | 31.524 | −11.968 | 59.851 | 1.00 | 40.54 | B |
| ATOM | 2560 | NZ | LYS | 357 | 32.455 | −12.795 | 60.666 | 1.00 | 40.76 | B |
| ATOM | 2561 | C | LYS | 357 | 27.816 | −9.447 | 62.552 | 1.00 | 33.43 | B |
| ATOM | 2562 | O | LYS | 357 | 26.911 | −9.512 | 61.724 | 1.00 | 33.00 | B |
| ATOM | 2563 | N | ASN | 358 | 27.654 | −9.773 | 63.833 | 1.00 | 34.87 | B |
| ATOM | 2564 | CA | ASN | 358 | 26.381 | −10.253 | 64.379 | 1.00 | 36.60 | B |
| ATOM | 2565 | CB | ASN | 358 | 26.621 | −10.942 | 65.724 | 1.00 | 37.20 | B |
| ATOM | 2566 | CG | ASN | 358 | 27.509 | −12.159 | 65.606 | 1.00 | 38.73 | B |
| ATOM | 2567 | OD1 | ASN | 358 | 28.105 | −12.602 | 66.589 | 1.00 | 40.28 | B |
| ATOM | 2568 | ND2 | ASN | 358 | 27.598 | −12.713 | 64.404 | 1.00 | 38.63 | B |
| ATOM | 2569 | C | ASN | 358 | 25.320 | −9.170 | 64.574 | 1.00 | 37.65 | B |
| ATOM | 2570 | O | ASN | 358 | 24.431 | −9.322 | 65.406 | 1.00 | 38.18 | B |
| ATOM | 2571 | N | ILE | 359 | 25.413 | −8.076 | 63.825 | 1.00 | 38.97 | B |
| ATOM | 2572 | CA | ILE | 359 | 24.430 | −7.003 | 63.951 | 1.00 | 40.85 | B |
| ATOM | 2573 | CB | ILE | 359 | 25.088 | −5.608 | 63.869 | 1.00 | 40.68 | B |
| ATOM | 2574 | CG2 | ILE | 359 | 24.014 | −4.529 | 63.858 | 1.00 | 40.16 | B |
| ATOM | 2575 | CG1 | ILE | 359 | 26.019 | −5.402 | 65.066 | 1.00 | 40.61 | B |
| ATOM | 2576 | CD1 | ILE | 359 | 26.871 | −4.161 | 64.970 | 1.00 | 39.58 | B |
| ATOM | 2577 | C | ILE | 359 | 23.391 | −7.132 | 62.847 | 1.00 | 41.96 | B |
| ATOM | 2578 | O | ILE | 359 | 23.729 | −7.227 | 61.671 | 1.00 | 42.22 | B |
| ATOM | 2579 | N | LEU | 360 | 22.122 | −7.140 | 63.241 | 1.00 | 43.88 | B |
| ATOM | 2580 | CA | LEU | 360 | 21.024 | −7.276 | 62.293 | 1.00 | 46.61 | B |
| ATOM | 2581 | CB | LEU | 360 | 19.952 | −8.212 | 62.864 | 1.00 | 48.74 | B |
| ATOM | 2582 | CG | LEU | 360 | 19.660 | −9.524 | 62.123 | 1.00 | 52.19 | B |
| ATOM | 2583 | CD1 | LEU | 360 | 18.886 | −10.456 | 63.043 | 1.00 | 51.91 | B |
| ATOM | 2584 | CD2 | LEU | 360 | 18.870 | −9.248 | 60.836 | 1.00 | 53.68 | B |
| ATOM | 2585 | C | LEU | 360 | 20.406 | −5.927 | 61.966 | 1.00 | 46.77 | B |
| ATOM | 2586 | O | LEU | 360 | 19.969 | −5.211 | 62.854 | 1.00 | 46.72 | B |
| ATOM | 2587 | N | ASN | 361 | 20.380 | −5.586 | 60.681 | 1.00 | 47.32 | B |
| ATOM | 2588 | CA | ASN | 361 | 19.805 | −4.320 | 60.242 | 1.00 | 48.31 | B |
| ATOM | 2589 | CB | ASN | 361 | 20.834 | −3.502 | 59.458 | 1.00 | 47.61 | B |
| ATOM | 2590 | CG | ASN | 361 | 21.795 | −2.743 | 60.360 | 1.00 | 48.03 | B |
| ATOM | 2591 | OD1 | ASN | 361 | 22.423 | −1.777 | 59.933 | 1.00 | 48.30 | B |
| ATOM | 2592 | ND2 | ASN | 361 | 21.913 | −3.175 | 61.609 | 1.00 | 47.01 | B |
| ATOM | 2593 | C | ASN | 361 | 18.563 | −4.526 | 59.387 | 1.00 | 49.65 | B |
| ATOM | 2594 | O | ASN | 361 | 18.294 | −5.627 | 58.919 | 1.00 | 51.43 | B |
| ATOM | 2595 | N | LYS | 362 | 17.821 | −3.443 | 59.180 | 1.00 | 51.11 | B |
| ATOM | 2596 | CA | LYS | 362 | 16.586 | −3.452 | 58.400 | 1.00 | 50.99 | B |
| ATOM | 2597 | CB | LYS | 362 | 16.883 | −3.545 | 56.896 | 1.00 | 50.83 | B |
| ATOM | 2598 | CG | LYS | 362 | 17.289 | −2.229 | 56.253 | 1.00 | 49.23 | B |
| ATOM | 2599 | CD | LYS | 362 | 17.117 | −2.268 | 54.740 | 1.00 | 48.73 | B |
| ATOM | 2600 | CE | LYS | 362 | 15.643 | −2.244 | 54.329 | 1.00 | 47.35 | B |
| ATOM | 2601 | NZ | LYS | 362 | 14.989 | −0.914 | 54.515 | 1.00 | 44.68 | B |
| ATOM | 2602 | C | LYS | 362 | 15.659 | −4.588 | 58.814 | 1.00 | 51.66 | B |
| ATOM | 2603 | O | LYS | 362 | 15.211 | −5.329 | 57.913 | 1.00 | 52.28 | B |
| ATOM | 2604 | OXT | LYS | 362 | 15.387 | −4.712 | 60.031 | 1.00 | 50.87 | B |
| ATOM | 2605 | MG | MG | 2602 | 43.651 | 10.621 | 59.419 | 1.00 | 27.37 | |
| ATOM | 2606 | PB | ADP | 2600 | 44.241 | 7.165 | 60.136 | 1.00 | 25.05 | ADP |
| ATOM | 2607 | O1B | ADP | 2600 | 44.666 | 7.765 | 61.419 | 1.00 | 26.27 | ADP |
| ATOM | 2608 | O2B | ADP | 2600 | 43.842 | 5.630 | 60.325 | 1.00 | 30.28 | ADP |
| ATOM | 2609 | O3B | ADP | 2600 | 43.097 | 7.920 | 59.552 | 1.00 | 28.27 | ADP |
| ATOM | 2610 | PA | ADP | 2600 | 45.608 | 7.818 | 57.697 | 1.00 | 39.43 | ADP |
| ATOM | 2611 | O1A | ADP | 2600 | 44.613 | 7.286 | 56.772 | 1.00 | 38.84 | ADP |
| ATOM | 2612 | O2A | ADP | 2600 | 45.462 | 9.276 | 57.778 | 1.00 | 41.49 | ADP |
| ATOM | 2613 | O3A | ADP | 2600 | 45.426 | 7.167 | 59.121 | 1.00 | 32.30 | ADP |
| ATOM | 2614 | O5* | ADP | 2600 | 47.084 | 7.550 | 57.187 | 1.00 | 39.41 | ADP |
| ATOM | 2615 | C5* | ADP | 2600 | 48.157 | 6.858 | 57.828 | 1.00 | 42.82 | ADP |
| ATOM | 2616 | C4* | ADP | 2600 | 49.374 | 6.940 | 56.825 | 1.00 | 45.97 | ADP |
| ATOM | 2617 | O4* | ADP | 2600 | 49.399 | 5.696 | 56.137 | 1.00 | 46.62 | ADP |
| ATOM | 2618 | C3* | ADP | 2600 | 49.266 | 8.021 | 55.715 | 1.00 | 46.20 | ADP |
| ATOM | 2619 | O3* | ADP | 2600 | 50.512 | 8.717 | 55.502 | 1.00 | 49.03 | ADP |
| ATOM | 2620 | C2* | ADP | 2600 | 48.810 | 7.296 | 54.462 | 1.00 | 46.75 | ADP |
| ATOM | 2621 | O2* | ADP | 2600 | 49.235 | 7.921 | 53.240 | 1.00 | 48.13 | ADP |
| ATOM | 2622 | C1* | ADP | 2600 | 49.328 | 5.886 | 54.701 | 1.00 | 47.35 | ADP |
| ATOM | 2623 | N9 | ADP | 2600 | 48.435 | 4.815 | 54.144 | 1.00 | 48.03 | ADP |
| ATOM | 2624 | C8 | ADP | 2600 | 47.417 | 4.221 | 54.811 | 1.00 | 47.72 | ADP |
| ATOM | 2625 | N7 | ADP | 2600 | 46.839 | 3.328 | 54.046 | 1.00 | 48.56 | ADP |
| ATOM | 2626 | C5 | ADP | 2600 | 47.454 | 3.316 | 52.892 | 1.00 | 49.10 | ADP |
| ATOM | 2627 | C6 | ADP | 2600 | 47.308 | 2.603 | 51.707 | 1.00 | 49.07 | ADP |
| ATOM | 2628 | N6 | ADP | 2600 | 46.350 | 1.680 | 51.610 | 1.00 | 49.43 | ADP |
| ATOM | 2629 | N1 | ADP | 2600 | 48.159 | 2.844 | 50.628 | 1.00 | 50.04 | ADP |
| ATOM | 2630 | C2 | ADP | 2600 | 49.152 | 3.776 | 50.684 | 1.00 | 48.98 | ADP |
| ATOM | 2631 | N3 | ADP | 2600 | 49.301 | 4.478 | 51.842 | 1.00 | 50.49 | ADP |
| ATOM | 2632 | C4 | ADP | 2600 | 48.491 | 4.283 | 52.944 | 1.00 | 48.96 | ADP |
| ATOM | 2633 | C1 | 2-7 | 1 | 37.376 | 16.487 | 53.441 | 1.00 | 31.12 | 2-7 |
| ATOM | 2634 | C2 | 2-7 | 1 | 38.554 | 16.442 | 52.639 | 1.00 | 31.01 | 2-7 |
| ATOM | 2635 | C3 | 2-7 | 1 | 38.554 | 15.433 | 51.622 | 1.00 | 31.01 | 2-7 |
| ATOM | 2636 | C4 | 2-7 | 1 | 37.388 | 14.559 | 51.530 | 1.00 | 29.91 | 2-7 |
| ATOM | 2637 | C5 | 2-7 | 1 | 36.248 | 14.570 | 52.396 | 1.00 | 29.25 | 2-7 |
| ATOM | 2638 | C6 | 2-7 | 1 | 36.296 | 15.546 | 53.415 | 1.00 | 30.61 | 2-7 |
| ATOM | 2639 | C10 | 2-7 | 1 | 39.708 | 15.357 | 50.686 | 1.00 | 30.99 | 2-7 |
| ATOM | 2640 | C11 | 2-7 | 1 | 40.272 | 16.598 | 50.056 | 1.00 | 33.35 | 2-7 |
| ATOM | 2641 | N12 | 2-7 | 1 | 41.446 | 16.158 | 49.317 | 1.00 | 33.73 | 2-7 |
| ATOM | 2642 | C13 | 2-7 | 1 | 41.189 | 14.730 | 49.013 | 1.00 | 31.60 | 2-7 |
| ATOM | 2643 | C14 | 2-7 | 1 | 40.419 | 14.175 | 50.202 | 1.00 | 30.03 | 2-7 |
| ATOM | 2644 | C17 | 2-7 | 1 | 41.032 | 14.136 | 47.645 | 1.00 | 28.72 | 2-7 |
| ATOM | 2645 | C19 | 2-7 | 1 | 42.014 | 13.131 | 47.164 | 1.00 | 27.73 | 2-7 |
| ATOM | 2646 | C20 | 2-7 | 1 | 41.952 | 12.752 | 45.765 | 1.00 | 26.29 | 2-7 |
| ATOM | 2647 | C21 | 2-7 | 1 | 40.984 | 13.380 | 44.878 | 1.00 | 26.40 | 2-7 |
| ATOM | 2648 | C22 | 2-7 | 1 | 39.931 | 14.256 | 45.351 | 1.00 | 27.79 | 2-7 |
| ATOM | 2649 | C23 | 2-7 | 1 | 39.958 | 14.694 | 46.762 | 1.00 | 27.64 | 2-7 |
| ATOM | 2650 | C29 | 2-7 | 1 | 42.438 | 17.110 | 49.102 | 1.00 | 34.81 | 2-7 |
| ATOM | 2651 | N30 | 2-7 | 1 | 43.717 | 16.767 | 49.283 | 1.00 | 35.06 | 2-7 |
| ATOM | 2652 | C31 | 2-7 | 1 | 44.603 | 17.929 | 49.086 | 1.00 | 31.67 | 2-7 |
| ATOM | 2653 | C35 | 2-7 | 1 | 44.177 | 15.446 | 49.734 | 1.00 | 32.58 | 2-7 |
| ATOM | 2654 | O39 | 2-7 | 1 | 42.187 | 18.279 | 48.762 | 1.00 | 35.09 | 2-7 |
| ATOM | 2655 | F40 | 2-7 | 1 | 37.369 | 13.692 | 50.535 | 1.00 | 32.42 | 2-7 |
| ATOM | 2656 | F41 | 2-7 | 1 | 37.291 | 17.497 | 54.277 | 1.00 | 33.09 | 2-7 |
| ATOM | 2657 | O | HOH | 2 | 38.630 | 10.603 | 62.535 | 1.00 | 3.96 | S |
| ATOM | 2658 | O | HOH | 3 | 28.064 | 20.853 | 56.798 | 1.00 | 15.26 | S |
| ATOM | 2659 | O | HOH | 4 | 43.423 | −1.052 | 63.682 | 1.00 | 6.84 | S |
| ATOM | 2660 | O | HOH | 5 | 41.471 | 9.650 | 60.748 | 1.00 | 28.56 | S |
| ATOM | 2661 | O | HOH | 6 | 53.043 | −17.874 | 61.146 | 1.00 | 22.21 | S |
| ATOM | 2662 | O | HOH | 8 | 43.351 | 23.546 | 43.947 | 1.00 | 14.88 | S |
| ATOM | 2663 | O | HOH | 11 | 31.538 | 6.420 | 79.791 | 1.00 | 20.07 | S |
| ATOM | 2664 | O | HOH | 12 | 44.364 | 1.570 | 53.833 | 1.00 | 33.76 | S |
| ATOM | 2665 | O | HOH | 13 | 42.141 | −0.803 | 71.483 | 1.00 | 23.37 | S |
| ATOM | 2666 | O | HOH | 17 | 50.048 | −0.508 | 68.644 | 1.00 | 38.33 | S |
| ATOM | 2667 | O | HOH | 18 | 42.525 | 8.183 | 64.075 | 1.00 | 31.71 | S |
| ATOM | 2668 | O | HOH | 20 | 49.961 | −5.304 | 63.635 | 1.00 | 28.76 | S |
| ATOM | 2669 | O | HOH | 21 | 52.974 | 11.228 | 41.771 | 1.00 | 27.37 | S |
| ATOM | 2670 | O | HOH | 23 | 44.880 | 17.208 | 64.490 | 1.00 | 19.87 | S |
| ATOM | 2671 | O | HOH | 25 | 33.865 | 11.390 | 57.228 | 1.00 | 14.50 | S |
| ATOM | 2672 | O | HOH | 26 | 42.746 | 19.345 | 56.865 | 1.00 | 19.80 | S |
| ATOM | 2673 | O | HOH | 27 | 43.217 | 3.216 | 42.636 | 1.00 | 29.84 | S |
| ATOM | 2674 | O | HOH | 28 | 47.542 | 18.783 | 69.096 | 1.00 | 24.56 | S |
| ATOM | 2675 | O | HOH | 29 | 29.606 | −8.997 | 58.639 | 1.00 | 41.51 | S |
| ATOM | 2676 | O | HOH | 30 | 38.143 | 15.249 | 61.346 | 1.00 | 12.36 | S |
| ATOM | 2677 | O | HOH | 31 | 47.769 | 14.311 | 41.568 | 1.00 | 24.48 | S |
| ATOM | 2678 | O | HOH | 32 | 22.227 | 19.477 | 42.995 | 1.00 | 35.68 | S |
| ATOM | 2679 | O | HOH | 34 | 38.077 | 4.715 | 80.434 | 1.00 | 19.14 | S |
| ATOM | 2680 | O | HOH | 35 | 27.208 | 25.794 | 60.457 | 1.00 | 30.49 | S |
| ATOM | 2681 | O | HOH | 40 | 45.874 | 21.711 | 68.966 | 1.00 | 14.93 | S |
| ATOM | 2682 | O | HOH | 42 | 37.931 | 3.241 | 64.945 | 1.00 | 21.80 | S |
| ATOM | 2683 | O | HOH | 44 | 33.173 | 12.293 | 71.900 | 1.00 | 38.67 | S |
| ATOM | 2684 | O | HOH | 45 | 38.986 | 3.636 | 49.470 | 1.00 | 20.20 | S |
| ATOM | 2685 | O | HOH | 46 | 35.162 | 19.890 | 41.213 | 1.00 | 25.42 | S |
| ATOM | 2686 | O | HOH | 52 | 22.755 | −3.615 | 56.949 | 1.00 | 33.63 | S |
| ATOM | 2687 | O | HOH | 53 | 27.917 | 6.206 | 79.432 | 1.00 | 19.49 | S |
| ATOM | 2688 | O | HOH | 55 | 37.862 | 4.182 | 47.024 | 1.00 | 13.89 | S |
| ATOM | 2689 | O | HOH | 57 | 31.462 | 4.272 | 82.519 | 1.00 | 37.59 | S |
| ATOM | 2690 | O | HOH | 59 | 38.826 | 12.586 | 58.140 | 1.00 | 18.34 | S |
| ATOM | 2691 | O | HOH | 60 | 27.879 | 4.380 | 76.644 | 1.00 | 24.90 | S |
| ATOM | 2692 | O | HOH | 61 | 45.041 | 10.037 | 53.740 | 1.00 | 42.66 | S |
| ATOM | 2693 | O | HOH | 62 | 28.763 | 26.533 | 62.454 | 1.00 | 35.09 | S |
| ATOM | 2694 | O | HOH | 66 | 38.448 | −0.512 | 37.739 | 1.00 | 44.71 | S |
| ATOM | 2695 | O | HOH | 67 | 31.394 | 24.733 | 63.775 | 1.00 | 40.50 | S |
| ATOM | 2696 | O | HOH | 68 | 40.487 | 5.787 | 72.041 | 1.00 | 37.21 | S |
| ATOM | 2697 | O | HOH | 69 | 52.548 | 19.976 | 38.009 | 1.00 | 24.27 | S |
| ATOM | 2698 | O | HOH | 70 | 40.043 | −1.641 | 68.804 | 1.00 | 21.10 | S |
| ATOM | 2699 | O | HOH | 71 | 21.370 | 18.117 | 39.097 | 1.00 | 47.89 | S |
| ATOM | 2700 | O | HOH | 73 | 45.431 | −1.388 | 51.309 | 1.00 | 36.21 | S |
| ATOM | 2701 | O | HOH | 74 | 12.109 | 0.216 | 54.870 | 1.00 | 45.32 | S |
| ATOM | 2702 | O | HOH | 78 | 41.390 | 5.467 | 40.236 | 1.00 | 31.36 | S |
| ATOM | 2703 | O | HOH | 79 | 38.398 | −10.202 | 49.709 | 1.00 | 28.25 | S |
| ATOM | 2704 | O | HOH | 84 | 46.457 | −1.971 | 63.989 | 1.00 | 20.69 | S |
| ATOM | 2705 | O | HOH | 87 | 2.291 | 6.433 | 36.064 | 1.00 | 27.27 | S |
| ATOM | 2706 | O | HOH | 88 | 46.187 | 3.359 | 74.292 | 1.00 | 30.60 | S |
| ATOM | 2707 | O | HOH | 89 | 51.911 | 4.577 | 56.634 | 1.00 | 44.94 | S |
| ATOM | 2708 | O | HOH | 90 | 45.811 | 18.580 | 66.703 | 1.00 | 26.87 | S |
| ATOM | 2709 | O | HOH | 91 | 47.734 | 13.013 | 72.702 | 1.00 | 32.94 | S |
| ATOM | 2710 | O | HOH | 92 | 23.555 | 15.386 | 53.064 | 1.00 | 29.56 | S |
| ATOM | 2711 | O | HOH | 93 | 43.670 | −2.643 | 73.172 | 1.00 | 27.18 | S |
| ATOM | 2712 | O | HOH | 94 | 27.978 | 20.947 | 70.487 | 1.00 | 41.48 | S |
| ATOM | 2713 | O | HOH | 95 | 44.678 | −7.048 | 71.862 | 1.00 | 24.48 | S |
| ATOM | 2714 | O | HOH | 97 | 37.124 | 2.776 | 73.009 | 1.00 | 36.39 | S |
| ATOM | 2715 | O | HOH | 98 | 32.730 | 25.500 | 47.607 | 1.00 | 42.43 | S |
| ATOM | 2716 | O | HOH | 101 | 46.793 | 22.739 | 62.116 | 1.00 | 28.62 | S |
| ATOM | 2717 | O | HOH | 104 | 20.079 | 21.304 | 46.635 | 1.00 | 44.83 | S |
| ATOM | 2718 | O | HOH | 105 | 30.653 | −3.670 | 75.744 | 1.00 | 35.11 | S |
| ATOM | 2719 | O | HOH | 106 | 46.987 | 13.182 | 34.815 | 1.00 | 16.99 | S |
| ATOM | 2720 | O | HOH | 109 | 43.794 | 0.066 | 55.803 | 1.00 | 30.02 | S |
| ATOM | 2721 | O | HOH | 111 | 25.208 | 9.102 | 28.662 | 1.00 | 32.86 | S |
| ATOM | 2722 | O | HOH | 113 | 44.655 | 15.401 | 59.741 | 1.00 | 25.68 | S |
| ATOM | 2723 | O | HOH | 115 | 18.285 | 12.456 | 33.587 | 1.00 | 30.40 | S |
| ATOM | 2724 | O | HOH | 116 | 47.999 | −0.217 | 48.915 | 1.00 | 36.92 | S |
| ATOM | 2725 | O | HOH | 117 | 23.508 | 25.313 | 66.864 | 1.00 | 47.95 | S |
| ATOM | 2726 | O | HOH | 119 | 27.220 | −14.904 | 55.904 | 1.00 | 35.41 | S |
| ATOM | 2727 | O | HOH | 120 | 47.343 | 8.255 | 68.520 | 1.00 | 37.89 | S |
| ATOM | 2728 | O | HOH | 128 | 28.608 | −6.298 | 48.882 | 1.00 | 26.00 | S |
| ATOM | 2729 | O | HOH | 132 | 6.107 | 15.208 | 42.672 | 1.00 | 30.09 | S |
| ATOM | 2730 | O | HOH | 133 | 26.812 | 14.766 | 57.900 | 1.00 | 17.88 | S |
| ATOM | 2731 | O | HOH | 135 | 46.950 | 10.746 | 67.779 | 1.00 | 31.59 | S |
| ATOM | 2732 | O | HOH | 136 | 24.332 | 1.606 | 79.565 | 1.00 | 28.86 | S |
| ATOM | 2733 | O | HOH | 138 | 50.215 | 2.473 | 62.680 | 1.00 | 35.95 | S |
| ATOM | 2734 | O | HOH | 139 | 22.069 | 24.748 | 54.683 | 1.00 | 25.56 | S |
| ATOM | 2735 | O | HOH | 140 | 44.497 | −18.491 | 58.486 | 1.00 | 49.65 | S |
| ATOM | 2736 | O | HOH | 141 | 15.900 | −4.594 | 62.687 | 1.00 | 33.93 | S |
| ATOM | 2737 | O | HOH | 143 | 14.793 | −3.866 | 47.507 | 1.00 | 45.81 | S |
| END | | | | | | | | | | |

TABLE 4
REMARK FILENAME = “Compound 4-2a_2dpb.pdb”
!CRYST 69.200 79.400 159.200 90.00 90.00 90.00 P212121

| | | | | | | | | | | |
|---|---|---|---|---|---|---|---|---|---|---|
| ATOM | 2605 | CB | LYS | 17 | 24.472 | −12.132 | 60.197 | 1.00 | 50.92 | B |
| ATOM | 2606 | CG | LYS | 17 | 23.137 | −12.714 | 59.720 | 1.00 | 53.46 | B |
| ATOM | 2607 | CD | LYS | 17 | 22.777 | −12.276 | 58.298 | 1.00 | 55.17 | B |
| ATOM | 2608 | CE | LYS | 17 | 23.486 | −13.129 | 57.240 | 1.00 | 56.45 | B |
| ATOM | 2609 | NZ | LYS | 17 | 24.977 | −13.074 | 57.341 | 1.00 | 55.91 | B |
| ATOM | 2610 | C | LYS | 17 | 24.464 | −9.793 | 59.322 | 1.00 | 46.31 | B |
| ATOM | 2611 | O | LYS | 17 | 25.371 | −9.870 | 58.525 | 1.00 | 47.38 | B |
| ATOM | 2612 | N | LYS | 17 | 23.273 | −10.326 | 61.434 | 1.00 | 49.07 | B |
| ATOM | 2613 | CA | LYS | 17 | 24.459 | −10.640 | 60.578 | 1.00 | 48.39 | B |
| ATOM | 2614 | N | ASN | 18 | 23.441 | −8.969 | 59.167 | 1.00 | 44.08 | B |
| ATOM | 2615 | CA | ASN | 18 | 23.346 | −8.128 | 57.990 | 1.00 | 42.08 | B |
| ATOM | 2616 | CB | ASN | 18 | 22.016 | −7.375 | 58.014 | 1.00 | 42.87 | B |
| ATOM | 2617 | CG | ASN | 18 | 21.059 | −7.856 | 56.934 | 1.00 | 45.64 | B |
| ATOM | 2618 | OD1 | ASN | 18 | 21.222 | −7.538 | 55.748 | 1.00 | 47.65 | B |
| ATOM | 2619 | ND2 | ASN | 18 | 20.068 | −8.642 | 57.331 | 1.00 | 46.01 | B |
| ATOM | 2620 | C | ASN | 18 | 24.508 | −7.150 | 57.750 | 1.00 | 40.28 | B |
| ATOM | 2621 | O | ASN | 18 | 24.895 | −6.921 | 56.596 | 1.00 | 42.10 | B |
| ATOM | 2622 | N | ILE | 19 | 25.077 | −6.584 | 58.810 | 1.00 | 36.30 | B |
| ATOM | 2623 | CA | ILE | 19 | 26.171 | −5.618 | 58.668 | 1.00 | 32.31 | B |
| ATOM | 2624 | CB | ILE | 19 | 26.495 | −4.982 | 60.043 | 1.00 | 33.05 | B |
| ATOM | 2625 | CG2 | ILE | 19 | 26.959 | −6.042 | 61.012 | 1.00 | 34.85 | B |
| ATOM | 2626 | CG1 | ILE | 19 | 27.599 | −3.938 | 59.905 | 1.00 | 33.89 | B |
| ATOM | 2627 | CD1 | ILE | 19 | 27.845 | −3.169 | 61.165 | 1.00 | 32.25 | B |
| ATOM | 2628 | C | ILE | 19 | 27.464 | −6.184 | 58.058 | 1.00 | 28.41 | B |
| ATOM | 2629 | O | ILE | 19 | 28.021 | −7.161 | 58.574 | 1.00 | 29.07 | B |
| ATOM | 2630 | N | GLN | 20 | 27.934 | −5.566 | 56.967 | 1.00 | 22.29 | B |
| ATOM | 2631 | CA | GLN | 20 | 29.174 | −5.986 | 56.285 | 1.00 | 15.95 | B |
| ATOM | 2632 | CB | GLN | 20 | 29.216 | −5.493 | 54.839 | 1.00 | 14.82 | B |
| ATOM | 2633 | CG | GLN | 20 | 30.526 | −5.834 | 54.127 | 1.00 | 14.68 | B |
| ATOM | 2634 | CD | GLN | 20 | 30.589 | −5.290 | 52.715 | 1.00 | 13.60 | B |
| ATOM | 2635 | OE1 | GLN | 20 | 30.540 | −4.089 | 52.514 | 1.00 | 13.47 | B |
| ATOM | 2636 | NE2 | GLN | 20 | 30.720 | −6.173 | 51.737 | 1.00 | 13.04 | B |
| ATOM | 2637 | C | GLN | 20 | 30.450 | −5.437 | 56.952 | 1.00 | 13.25 | B |
| ATOM | 2638 | O | GLN | 20 | 30.566 | −4.239 | 57.180 | 1.00 | 12.33 | B |
| ATOM | 2639 | N | VAL | 21 | 31.394 | −6.328 | 57.254 | 1.00 | 9.34 | B |
| ATOM | 2640 | CA | VAL | 21 | 32.656 | −5.941 | 57.880 | 1.00 | 6.24 | B |
| ATOM | 2641 | CB | VAL | 21 | 32.775 | −6.537 | 59.296 | 1.00 | 5.92 | B |
| ATOM | 2642 | CG1 | VAL | 21 | 34.094 | −6.144 | 59.934 | 1.00 | 3.44 | B |
| ATOM | 2643 | CG2 | VAL | 21 | 31.616 | −6.056 | 60.138 | 1.00 | 7.73 | B |
| ATOM | 2644 | C | VAL | 21 | 33.868 | −6.396 | 57.052 | 1.00 | 5.09 | B |
| ATOM | 2645 | O | VAL | 21 | 34.031 | −7.569 | 56.766 | 1.00 | 4.24 | B |
| ATOM | 2646 | N | VAL | 22 | 34.715 | −5.454 | 56.659 | 1.00 | 3.75 | B |
| ATOM | 2647 | CA | VAL | 22 | 35.893 | −5.805 | 55.879 | 1.00 | 4.12 | B |
| ATOM | 2648 | CB | VAL | 22 | 35.819 | −5.226 | 54.420 | 1.00 | 3.36 | B |
| ATOM | 2649 | CG1 | VAL | 22 | 34.566 | −5.731 | 53.703 | 1.00 | 3.16 | B |
| ATOM | 2650 | CG2 | VAL | 22 | 35.823 | −3.717 | 54.452 | 1.00 | 2.87 | B |
| ATOM | 2651 | C | VAL | 22 | 37.157 | −5.305 | 56.553 | 1.00 | 6.20 | B |
| ATOM | 2652 | O | VAL | 22 | 37.122 | −4.365 | 57.352 | 1.00 | 6.79 | B |
| ATOM | 2653 | N | VAL | 23 | 38.271 | −5.946 | 56.223 | 1.00 | 4.46 | B |
| ATOM | 2654 | CA | VAL | 23 | 39.559 | −5.585 | 56.785 | 1.00 | 4.23 | B |
| ATOM | 2655 | CB | VAL | 23 | 40.195 | −6.830 | 57.477 | 1.00 | 4.02 | B |
| ATOM | 2656 | CG1 | VAL | 23 | 41.555 | −6.511 | 58.081 | 1.00 | 1.86 | B |
| ATOM | 2657 | CG2 | VAL | 23 | 39.268 | −7.319 | 58.550 | 1.00 | 5.77 | B |
| ATOM | 2658 | C | VAL | 23 | 40.505 | −5.037 | 55.710 | 1.00 | 4.46 | B |
| ATOM | 2659 | O | VAL | 23 | 40.553 | −5.531 | 54.586 | 1.00 | 4.66 | B |
| ATOM | 2660 | N | ARG | 24 | 41.251 | −3.998 | 56.057 | 1.00 | 7.29 | B |
| ATOM | 2661 | CA | ARG | 24 | 42.228 | −3.436 | 55.128 | 1.00 | 9.87 | B |
| ATOM | 2662 | CB | ARG | 24 | 41.793 | −2.092 | 54.531 | 1.00 | 6.53 | B |
| ATOM | 2663 | CG | ARG | 24 | 42.744 | −1.662 | 53.425 | 1.00 | 6.89 | B |
| ATOM | 2664 | CD | ARG | 24 | 42.401 | −0.306 | 52.837 | 1.00 | 7.91 | B |
| ATOM | 2665 | NE | ARG | 24 | 43.142 | −0.040 | 51.603 | 1.00 | 4.86 | B |
| ATOM | 2666 | CZ | ARG | 24 | 43.041 | 1.095 | 50.909 | 1.00 | 3.46 | B |
| ATOM | 2667 | NH1 | ARG | 24 | 42.228 | 2.056 | 51.329 | 1.00 | 1.00 | B |
| ATOM | 2668 | NH2 | ARG | 24 | 43.773 | 1.287 | 49.814 | 1.00 | 1.00 | B |
| ATOM | 2669 | C | ARG | 24 | 43.541 | −3.179 | 55.856 | 1.00 | 13.03 | B |
| ATOM | 2670 | O | ARG | 24 | 43.586 | −2.374 | 56.791 | 1.00 | 13.45 | B |
| ATOM | 2671 | N | CYS | 25 | 44.593 | −3.873 | 55.421 | 1.00 | 13.86 | B |
| ATOM | 2672 | CA | CYS | 25 | 45.928 | −3.742 | 55.996 | 1.00 | 16.78 | B |
| ATOM | 2673 | CB | CYS | 25 | 46.646 | −5.088 | 55.932 | 1.00 | 14.53 | B |
| ATOM | 2674 | SG | CYS | 25 | 48.149 | −5.147 | 56.865 | 1.00 | 15.92 | B |
| ATOM | 2675 | C | CYS | 25 | 46.743 | −2.706 | 55.216 | 1.00 | 17.93 | B |
| ATOM | 2676 | O | CYS | 25 | 46.793 | −2.743 | 53.991 | 1.00 | 19.83 | B |
| ATOM | 2677 | N | ARG | 26 | 47.369 | −1.774 | 55.922 | 1.00 | 20.13 | B |
| ATOM | 2678 | CA | ARG | 26 | 48.186 | −0.779 | 55.242 | 1.00 | 23.56 | B |
| ATOM | 2679 | CB | ARG | 26 | 48.410 | 0.441 | 56.122 | 1.00 | 23.04 | B |
| ATOM | 2680 | CG | ARG | 26 | 49.018 | 0.108 | 57.480 | 1.00 | 25.34 | B |
| ATOM | 2681 | CD | ARG | 26 | 49.478 | 1.335 | 58.248 | 1.00 | 25.85 | B |
| ATOM | 2682 | NE | ARG | 26 | 50.882 | 1.635 | 57.970 | 1.00 | 27.66 | B |
| ATOM | 2683 | CZ | ARG | 26 | 51.876 | 1.425 | 58.830 | 1.00 | 29.35 | B |
| ATOM | 2684 | NH1 | ARG | 26 | 51.620 | 0.914 | 60.030 | 1.00 | 28.00 | B |
| ATOM | 2685 | NH2 | ARG | 26 | 53.126 | 1.729 | 58.494 | 1.00 | 29.65 | B |
| ATOM | 2686 | C | ARG | 26 | 49.566 | −1.360 | 54.924 | 1.00 | 26.17 | B |
| ATOM | 2687 | O | ARG | 26 | 49.965 | −2.367 | 55.500 | 1.00 | 27.47 | B |
| ATOM | 2688 | N | PRO | 27 | 50.296 | −0.748 | 53.976 | 1.00 | 28.46 | B |
| ATOM | 2689 | CD | PRO | 27 | 49.815 | 0.221 | 52.972 | 1.00 | 28.96 | B |
| ATOM | 2690 | CA | PRO | 27 | 51.634 | −1.225 | 53.617 | 1.00 | 30.05 | B |
| ATOM | 2691 | CB | PRO | 27 | 51.757 | −0.791 | 52.157 | 1.00 | 29.21 | B |
| ATOM | 2692 | CG | PRO | 27 | 51.081 | 0.508 | 52.153 | 1.00 | 27.78 | B |
| ATOM | 2693 | C | PRO | 27 | 52.652 | −0.565 | 54.551 | 1.00 | 30.74 | B |
| ATOM | 2694 | O | PRO | 27 | 52.315 | 0.387 | 55.255 | 1.00 | 30.33 | B |
| ATOM | 2695 | N | PHE | 28 | 53.888 | −1.065 | 54.559 | 1.00 | 33.00 | B |
| ATOM | 2696 | CA | PHE | 28 | 54.946 | −0.488 | 55.397 | 1.00 | 35.47 | B |
| ATOM | 2697 | CB | PHE | 28 | 56.197 | −1.349 | 55.423 | 1.00 | 34.78 | B |
| ATOM | 2698 | CG | PHE | 28 | 56.043 | −2.621 | 56.180 | 1.00 | 34.30 | B |
| ATOM | 2699 | CD1 | PHE | 28 | 55.970 | −3.848 | 55.506 | 1.00 | 33.11 | B |
| ATOM | 2700 | CD2 | PHE | 28 | 55.975 | −2.598 | 57.566 | 1.00 | 34.50 | B |
| ATOM | 2701 | CE1 | PHE | 28 | 55.831 | −5.030 | 56.204 | 1.00 | 32.04 | B |
| ATOM | 2702 | CE2 | PHE | 28 | 55.833 | −3.779 | 58.283 | 1.00 | 34.83 | B |
| ATOM | 2703 | CZ | PHE | 28 | 55.762 | −5.002 | 57.594 | 1.00 | 34.76 | B |
| ATOM | 2704 | C | PHE | 28 | 55.432 | 0.848 | 54.837 | 1.00 | 37.44 | B |
| ATOM | 2705 | O | PHE | 28 | 55.529 | 1.019 | 53.640 | 1.00 | 37.96 | B |
| ATOM | 2706 | N | ASN | 29 | 55.724 | 1.797 | 55.719 | 1.00 | 41.21 | B |
| ATOM | 2707 | CA | ASN | 29 | 56.195 | 3.114 | 55.288 | 1.00 | 43.97 | B |
| ATOM | 2708 | CB | ASN | 29 | 55.731 | 4.190 | 56.280 | 1.00 | 42.30 | B |
| ATOM | 2709 | CG | ASN | 29 | 56.080 | 3.843 | 57.724 | 1.00 | 41.84 | B |
| ATOM | 2710 | OD1 | ASN | 29 | 57.230 | 3.554 | 58.038 | 1.00 | 40.87 | B |
| ATOM | 2711 | ND2 | ASN | 29 | 55.080 | 3.866 | 58.604 | 1.00 | 40.16 | B |
| ATOM | 2712 | C | ASN | 29 | 57.718 | 3.112 | 55.190 | 1.00 | 47.03 | B |
| ATOM | 2713 | O | ASN | 29 | 58.361 | 2.179 | 55.651 | 1.00 | 48.57 | B |
| ATOM | 2714 | N | LEU | 30 | 58.290 | 4.156 | 54.594 | 1.00 | 49.85 | B |
| ATOM | 2715 | CA | LEU | 30 | 59.745 | 4.258 | 54.442 | 1.00 | 52.56 | B |
| ATOM | 2716 | CB | LEU | 30 | 60.125 | 5.641 | 53.928 | 1.00 | 52.63 | B |
| ATOM | 2717 | CG | LEU | 30 | 60.214 | 5.735 | 52.409 | 1.00 | 53.20 | B |
| ATOM | 2718 | CD1 | LEU | 30 | 60.395 | 7.194 | 51.973 | 1.00 | 53.18 | B |
| ATOM | 2719 | CD2 | LEU | 30 | 61.378 | 4.862 | 51.935 | 1.00 | 54.30 | B |
| ATOM | 2720 | C | LEU | 30 | 60.579 | 3.978 | 55.695 | 1.00 | 54.36 | B |
| ATOM | 2721 | O | LEU | 30 | 61.623 | 3.347 | 55.619 | 1.00 | 54.97 | B |
| ATOM | 2722 | N | ALA | 31 | 60.121 | 4.453 | 56.847 | 1.00 | 56.36 | B |
| ATOM | 2723 | CA | ALA | 31 | 60.843 | 4.228 | 58.097 | 1.00 | 58.76 | B |
| ATOM | 2724 | CB | ALA | 31 | 60.214 | 5.057 | 59.202 | 1.00 | 58.55 | B |
| ATOM | 2725 | C | ALA | 31 | 60.842 | 2.742 | 58.487 | 1.00 | 60.40 | B |
| ATOM | 2726 | O | ALA | 31 | 61.749 | 2.266 | 59.167 | 1.00 | 60.67 | B |
| ATOM | 2727 | N | GLU | 32 | 59.819 | 2.016 | 58.045 | 1.00 | 61.95 | B |
| ATOM | 2728 | CA | GLU | 32 | 59.692 | 0.594 | 58.350 | 1.00 | 63.39 | B |
| ATOM | 2729 | CB | GLU | 32 | 58.215 | 0.187 | 58.322 | 1.00 | 62.91 | B |
| ATOM | 2730 | CG | GLU | 32 | 57.429 | 0.683 | 59.524 | 1.00 | 62.16 | B |
| ATOM | 2731 | CD | GLU | 32 | 55.933 | 0.669 | 59.299 | 1.00 | 61.37 | B |
| ATOM | 2732 | OE1 | GLU | 32 | 55.191 | 0.841 | 60.289 | 1.00 | 60.97 | B |
| ATOM | 2733 | OE2 | GLU | 32 | 55.504 | 0.497 | 58.138 | 1.00 | 60.36 | B |
| ATOM | 2734 | C | GLU | 32 | 60.487 | −0.318 | 57.414 | 1.00 | 64.76 | B |
| ATOM | 2735 | O | GLU | 32 | 61.130 | −1.261 | 57.860 | 1.00 | 64.21 | B |
| ATOM | 2736 | N | ARG | 33 | 60.436 | −0.039 | 56.116 | 1.00 | 66.90 | B |
| ATOM | 2737 | CA | ARG | 33 | 61.150 | −0.855 | 55.141 | 1.00 | 69.19 | B |
| ATOM | 2738 | CB | ARG | 33 | 60.690 | −0.503 | 53.719 | 1.00 | 70.74 | B |
| ATOM | 2739 | CG | ARG | 33 | 60.911 | 0.953 | 53.310 | 1.00 | 73.78 | B |
| ATOM | 2740 | CD | ARG | 33 | 60.238 | 1.267 | 51.977 | 1.00 | 75.17 | B |
| ATOM | 2741 | NE | ARG | 33 | 60.663 | 0.349 | 50.920 | 1.00 | 76.52 | B |
| ATOM | 2742 | CZ | ARG | 33 | 61.889 | 0.301 | 50.400 | 1.00 | 76.92 | B |
| ATOM | 2743 | NH1 | ARG | 33 | 62.838 | 1.122 | 50.829 | 1.00 | 76.57 | B |
| ATOM | 2744 | NH2 | ARG | 33 | 62.168 | −0.569 | 49.441 | 1.00 | 78.04 | B |
| ATOM | 2745 | C | ARG | 33 | 62.650 | −0.654 | 55.297 | 1.00 | 70.11 | B |
| ATOM | 2746 | O | ARG | 33 | 63.439 | −1.524 | 54.943 | 1.00 | 70.36 | B |
| ATOM | 2747 | N | LYS | 34 | 63.038 | 0.500 | 55.832 | 1.00 | 71.13 | B |
| ATOM | 2748 | CA | LYS | 34 | 64.447 | 0.798 | 56.053 | 1.00 | 72.18 | B |
| ATOM | 2749 | CB | LYS | 34 | 64.623 | 2.254 | 56.498 | 1.00 | 73.21 | B |
| ATOM | 2750 | CG | LYS | 34 | 64.611 | 3.267 | 55.363 | 1.00 | 74.27 | B |
| ATOM | 2751 | CD | LYS | 34 | 66.023 | 3.637 | 54.921 | 1.00 | 74.99 | B |
| ATOM | 2752 | CE | LYS | 34 | 66.769 | 2.463 | 54.306 | 1.00 | 74.88 | B |
| ATOM | 2753 | NZ | LYS | 34 | 68.154 | 2.852 | 53.916 | 1.00 | 75.81 | B |
| ATOM | 2754 | C | LYS | 34 | 65.006 | −0.137 | 57.123 | 1.00 | 72.12 | B |
| ATOM | 2755 | O | LYS | 34 | 66.207 | −0.424 | 57.142 | 1.00 | 72.82 | B |
| ATOM | 2756 | N | ALA | 35 | 64.130 | −0.612 | 58.007 | 1.00 | 71.37 | B |
| ATOM | 2757 | CA | ALA | 35 | 64.522 | −1.526 | 59.077 | 1.00 | 69.94 | B |
| ATOM | 2758 | CB | ALA | 35 | 63.780 | −1.177 | 60.361 | 1.00 | 69.77 | B |
| ATOM | 2759 | C | ALA | 35 | 64.223 | −2.970 | 58.685 | 1.00 | 69.24 | B |
| ATOM | 2760 | O | ALA | 35 | 64.198 | −3.854 | 59.542 | 1.00 | 69.32 | B |
| ATOM | 2761 | N | SER | 36 | 64.001 | −3.194 | 57.388 | 1.00 | 68.43 | B |
| ATOM | 2762 | CA | SER | 36 | 63.689 | −4.519 | 56.848 | 1.00 | 66.99 | B |
| ATOM | 2763 | CB | SER | 36 | 64.937 | −5.405 | 56.860 | 1.00 | 67.27 | B |
| ATOM | 2764 | OG | SER | 36 | 65.906 | −4.912 | 55.959 | 1.00 | 67.40 | B |
| ATOM | 2765 | C | SER | 36 | 62.579 | −5.159 | 57.674 | 1.00 | 65.70 | B |
| ATOM | 2766 | O | SER | 36 | 62.721 | −6.270 | 58.185 | 1.00 | 65.65 | B |
| ATOM | 2767 | N | ALA | 37 | 61.469 | −4.435 | 57.791 | 1.00 | 64.41 | B |
| ATOM | 2768 | CA | ALA | 37 | 60.320 | −4.880 | 58.568 | 1.00 | 62.00 | B |
| ATOM | 2769 | CB | ALA | 37 | 59.256 | −3.784 | 58.601 | 1.00 | 62.35 | B |
| ATOM | 2770 | C | ALA | 37 | 59.699 | −6.185 | 58.093 | 1.00 | 59.79 | B |
| ATOM | 2771 | O | ALA | 37 | 59.490 | −6.404 | 56.909 | 1.00 | 58.90 | B |
| ATOM | 2772 | N | HIS | 38 | 59.400 | −7.042 | 59.061 | 1.00 | 58.16 | B |
| ATOM | 2773 | CA | HIS | 38 | 58.795 | −8.347 | 58.828 | 1.00 | 55.57 | B |
| ATOM | 2774 | CB | HIS | 38 | 59.420 | −9.381 | 59.785 | 1.00 | 57.59 | B |
| ATOM | 2775 | CG | HIS | 38 | 59.426 | −8.963 | 61.233 | 1.00 | 58.97 | B |
| ATOM | 2776 | CD2 | HIS | 38 | 58.878 | −9.543 | 62.328 | 1.00 | 58.78 | B |
| ATOM | 2777 | ND1 | HIS | 38 | 60.083 | −7.837 | 61.689 | 1.00 | 58.86 | B |
| ATOM | 2778 | CE1 | HIS | 38 | 59.939 | −7.744 | 63.000 | 1.00 | 58.84 | B |
| ATOM | 2779 | NE2 | HIS | 38 | 59.211 | −8.766 | 63.412 | 1.00 | 58.91 | B |
| ATOM | 2780 | C | HIS | 38 | 57.296 | −8.223 | 59.086 | 1.00 | 53.05 | B |
| ATOM | 2781 | O | HIS | 38 | 56.890 | −7.787 | 60.163 | 1.00 | 54.10 | B |
| ATOM | 2782 | N | SER | 39 | 56.472 | −8.605 | 58.114 | 1.00 | 48.25 | B |
| ATOM | 2783 | CA | SER | 39 | 55.026 | −8.500 | 58.290 | 1.00 | 42.98 | B |
| ATOM | 2784 | CB | SER | 39 | 54.295 | −8.575 | 56.970 | 1.00 | 42.55 | B |
| ATOM | 2785 | OG | SER | 39 | 52.903 | −8.490 | 57.201 | 1.00 | 39.13 | B |
| ATOM | 2786 | C | SER | 39 | 54.444 | −9.616 | 59.130 | 1.00 | 40.52 | B |
| ATOM | 2787 | O | SER | 39 | 54.750 | −10.773 | 58.919 | 1.00 | 39.58 | B |
| ATOM | 2788 | N | ILE | 40 | 53.603 | −9.247 | 60.092 | 1.00 | 38.79 | B |
| ATOM | 2789 | CA | ILE | 40 | 52.967 | −10.222 | 60.979 | 1.00 | 36.32 | B |
| ATOM | 2790 | CB | ILE | 40 | 53.039 | −9.786 | 62.478 | 1.00 | 37.00 | B |
| ATOM | 2791 | CG2 | ILE | 40 | 54.493 | −9.677 | 62.925 | 1.00 | 37.72 | B |
| ATOM | 2792 | CG1 | ILE | 40 | 52.307 | −8.458 | 62.692 | 1.00 | 37.68 | B |
| ATOM | 2793 | CD1 | ILE | 40 | 52.102 | −8.097 | 64.161 | 1.00 | 37.35 | B |
| ATOM | 2794 | C | ILE | 40 | 51.501 | −10.426 | 60.611 | 1.00 | 34.00 | B |
| ATOM | 2795 | O | ILE | 40 | 50.757 | −11.084 | 61.319 | 1.00 | 32.93 | B |
| ATOM | 2796 | N | VAL | 41 | 51.097 | −9.863 | 59.482 | 1.00 | 33.39 | B |
| ATOM | 2797 | CA | VAL | 41 | 49.720 | −9.986 | 59.028 | 1.00 | 32.21 | B |
| ATOM | 2798 | CB | VAL | 41 | 48.982 | −8.617 | 59.042 | 1.00 | 31.99 | B |
| ATOM | 2799 | CG1 | VAL | 41 | 47.559 | −8.778 | 58.536 | 1.00 | 30.52 | B |
| ATOM | 2800 | CG2 | VAL | 41 | 48.964 | −8.048 | 60.445 | 1.00 | 32.73 | B |
| ATOM | 2801 | C | VAL | 41 | 49.685 | −10.526 | 57.610 | 1.00 | 32.35 | B |
| ATOM | 2802 | O | VAL | 41 | 50.357 | −10.022 | 56.728 | 1.00 | 31.91 | B |
| ATOM | 2803 | N | GLU | 42 | 48.886 | −11.565 | 57.417 | 1.00 | 33.52 | B |
| ATOM | 2804 | CA | GLU | 42 | 48.727 | −12.189 | 56.112 | 1.00 | 34.79 | B |
| ATOM | 2805 | CB | GLU | 42 | 49.228 | −13.626 | 56.142 | 1.00 | 34.88 | B |
| ATOM | 2806 | CG | GLU | 42 | 50.715 | −13.762 | 55.882 | 1.00 | 35.91 | B |
| ATOM | 2807 | CD | GLU | 42 | 51.222 | −15.139 | 56.222 | 1.00 | 36.62 | B |
| ATOM | 2808 | OE1 | GLU | 42 | 50.467 | −16.105 | 55.996 | 1.00 | 35.55 | B |
| ATOM | 2809 | OE2 | GLU | 42 | 52.373 | −15.262 | 56.704 | 1.00 | 36.67 | B |
| ATOM | 2810 | C | GLU | 42 | 47.264 | −12.207 | 55.689 | 1.00 | 34.67 | B |
| ATOM | 2811 | O | GLU | 42 | 46.425 | −12.745 | 56.388 | 1.00 | 35.11 | B |
| ATOM | 2812 | N | CYS | 43 | 46.959 | −11.615 | 54.540 | 1.00 | 33.53 | B |
| ATOM | 2813 | CA | CYS | 43 | 45.581 | −11.575 | 54.074 | 1.00 | 33.64 | B |
| ATOM | 2814 | CB | CYS | 43 | 45.241 | −10.172 | 53.575 | 1.00 | 31.73 | B |
| ATOM | 2815 | SG | CYS | 43 | 45.291 | −8.913 | 54.863 | 1.00 | 30.24 | B |
| ATOM | 2816 | C | CYS | 43 | 45.306 | −12.597 | 52.985 | 1.00 | 34.68 | B |
| ATOM | 2817 | O | CYS | 43 | 46.052 | −12.722 | 52.025 | 1.00 | 35.47 | B |
| ATOM | 2818 | N | ASP | 44 | 44.220 | −13.335 | 53.160 | 1.00 | 34.51 | B |
| ATOM | 2819 | CA | ASP | 44 | 43.821 | −14.347 | 52.196 | 1.00 | 35.72 | B |
| ATOM | 2820 | CB | ASP | 44 | 43.698 | −15.710 | 52.875 | 1.00 | 37.74 | B |
| ATOM | 2821 | CG | ASP | 44 | 43.627 | −16.858 | 51.880 | 1.00 | 39.14 | B |
| ATOM | 2822 | OD1 | ASP | 44 | 43.029 | −16.681 | 50.787 | 1.00 | 38.15 | B |
| ATOM | 2823 | OD2 | ASP | 44 | 44.166 | −17.941 | 52.206 | 1.00 | 40.23 | B |
| ATOM | 2824 | C | ASP | 44 | 42.452 | −13.949 | 51.662 | 1.00 | 36.02 | B |
| ATOM | 2825 | O | ASP | 44 | 41.433 | −14.323 | 52.228 | 1.00 | 34.41 | B |
| ATOM | 2826 | N | PRO | 45 | 42.415 | −13.177 | 50.566 | 1.00 | 36.48 | B |
| ATOM | 2827 | CD | PRO | 45 | 43.558 | −12.792 | 49.725 | 1.00 | 37.08 | B |
| ATOM | 2828 | CA | PRO | 45 | 41.162 | −12.727 | 49.962 | 1.00 | 36.44 | B |
| ATOM | 2829 | CB | PRO | 45 | 41.646 | −11.834 | 48.828 | 1.00 | 36.90 | B |
| ATOM | 2830 | CG | PRO | 45 | 42.892 | −12.518 | 48.398 | 1.00 | 37.61 | B |
| ATOM | 2831 | C | PRO | 45 | 40.254 | −13.872 | 49.518 | 1.00 | 36.95 | B |
| ATOM | 2832 | O | PRO | 45 | 39.046 | −13.805 | 49.685 | 1.00 | 37.27 | B |
| ATOM | 2833 | N | VAL | 46 | 40.834 | −14.912 | 48.930 | 1.00 | 37.39 | B |
| ATOM | 2834 | CA | VAL | 46 | 40.051 | −16.057 | 48.479 | 1.00 | 37.62 | B |
| ATOM | 2835 | CB | VAL | 46 | 40.943 | −17.087 | 47.773 | 1.00 | 38.49 | B |
| ATOM | 2836 | CG1 | VAL | 46 | 40.099 | −18.269 | 47.334 | 1.00 | 39.31 | B |
| ATOM | 2837 | CG2 | VAL | 46 | 41.642 | −16.436 | 46.584 | 1.00 | 38.33 | B |
| ATOM | 2838 | C | VAL | 46 | 39.354 | −16.728 | 49.665 | 1.00 | 37.65 | B |
| ATOM | 2839 | O | VAL | 46 | 38.172 | −17.082 | 49.606 | 1.00 | 38.03 | B |
| ATOM | 2840 | N | ARG | 47 | 40.089 | −16.902 | 50.752 | 1.00 | 37.10 | B |
| ATOM | 2841 | CA | ARG | 47 | 39.520 | −17.512 | 51.947 | 1.00 | 37.76 | B |
| ATOM | 2842 | CB | ARG | 47 | 40.627 | −18.142 | 52.797 | 1.00 | 40.98 | B |
| ATOM | 2843 | CG | ARG | 47 | 40.138 | −19.170 | 53.811 | 1.00 | 45.53 | B |
| ATOM | 2844 | CD | ARG | 47 | 40.088 | −20.569 | 53.205 | 1.00 | 48.08 | B |
| ATOM | 2845 | NE | ARG | 47 | 41.427 | −21.065 | 52.905 | 1.00 | 51.05 | B |
| ATOM | 2846 | CZ | ARG | 47 | 42.361 | −21.291 | 53.826 | 1.00 | 53.04 | B |
| ATOM | 2847 | NH1 | ARG | 47 | 42.101 | −21.066 | 55.108 | 1.00 | 53.32 | B |
| ATOM | 2848 | NH2 | ARG | 47 | 43.558 | −21.744 | 53.467 | 1.00 | 53.55 | B |
| ATOM | 2849 | C | ARG | 47 | 38.817 | −16.436 | 52.774 | 1.00 | 35.87 | B |
| ATOM | 2850 | O | ARG | 47 | 38.091 | −16.734 | 53.702 | 1.00 | 35.14 | B |
| ATOM | 2851 | N | LYS | 48 | 39.054 | −15.178 | 52.420 | 1.00 | 34.57 | B |
| ATOM | 2852 | CA | LYS | 48 | 38.456 | −14.051 | 53.125 | 1.00 | 32.91 | B |
| ATOM | 2853 | CB | LYS | 48 | 36.938 | −14.158 | 53.092 | 1.00 | 34.16 | B |
| ATOM | 2854 | CG | LYS | 48 | 36.361 | −14.145 | 51.693 | 1.00 | 36.73 | B |
| ATOM | 2855 | CD | LYS | 48 | 34.854 | −14.249 | 51.706 | 1.00 | 37.41 | B |
| ATOM | 2856 | CE | LYS | 48 | 34.338 | −14.550 | 50.314 | 1.00 | 38.70 | B |
| ATOM | 2857 | NZ | LYS | 48 | 34.704 | −13.479 | 49.344 | 1.00 | 36.20 | B |
| ATOM | 2858 | C | LYS | 48 | 38.903 | −13.978 | 54.578 | 1.00 | 31.33 | B |
| ATOM | 2859 | O | LYS | 48 | 38.140 | −13.593 | 55.440 | 1.00 | 31.50 | B |
| ATOM | 2860 | N | GLU | 49 | 40.151 | −14.352 | 54.836 | 1.00 | 29.95 | B |
| ATOM | 2861 | CA | GLU | 49 | 40.692 | −14.330 | 56.193 | 1.00 | 27.26 | B |
| ATOM | 2862 | CB | GLU | 49 | 41.168 | −15.719 | 56.633 | 1.00 | 28.44 | B |
| ATOM | 2863 | CG | GLU | 49 | 40.135 | −16.815 | 56.656 | 1.00 | 28.64 | B |
| ATOM | 2864 | CD | GLU | 49 | 40.760 | −18.160 | 56.980 | 1.00 | 29.46 | B |
| ATOM | 2865 | OE1 | GLU | 49 | 40.028 | −19.168 | 56.992 | 1.00 | 29.37 | B |
| ATOM | 2866 | OE2 | GLU | 49 | 41.986 | −18.211 | 57.220 | 1.00 | 29.95 | B |
| ATOM | 2867 | C | GLU | 49 | 41.924 | −13.438 | 56.344 | 1.00 | 24.62 | B |
| ATOM | 2868 | O | GLU | 49 | 42.648 | −13.164 | 55.395 | 1.00 | 23.41 | B |
| ATOM | 2869 | N | VAL | 50 | 42.123 | −12.973 | 57.565 | 1.00 | 23.85 | B |
| ATOM | 2870 | CA | VAL | 50 | 43.276 | −12.164 | 57.915 | 1.00 | 22.58 | B |
| ATOM | 2871 | CB | VAL | 50 | 42.852 | −10.738 | 58.417 | 1.00 | 21.03 | B |
| ATOM | 2872 | CG1 | VAL | 50 | 41.863 | −10.851 | 59.540 | 1.00 | 20.58 | B |
| ATOM | 2873 | CG2 | VAL | 50 | 44.047 | −9.968 | 58.884 | 1.00 | 19.55 | B |
| ATOM | 2874 | C | VAL | 50 | 43.909 | −12.995 | 59.036 | 1.00 | 23.21 | B |
| ATOM | 2875 | O | VAL | 50 | 43.234 | −13.410 | 59.959 | 1.00 | 22.47 | B |
| ATOM | 2876 | N | SER | 51 | 45.197 | −13.286 | 58.923 | 1.00 | 24.22 | B |
| ATOM | 2877 | CA | SER | 51 | 45.867 | −14.078 | 59.950 | 1.00 | 26.05 | B |
| ATOM | 2878 | CB | SER | 51 | 46.398 | −15.380 | 59.352 | 1.00 | 26.43 | B |
| ATOM | 2879 | OG | SER | 51 | 46.705 | −16.299 | 60.383 | 1.00 | 26.88 | B |
| ATOM | 2880 | C | SER | 51 | 47.013 | −13.293 | 60.579 | 1.00 | 26.62 | B |
| ATOM | 2881 | O | SER | 51 | 47.893 | −12.781 | 59.868 | 1.00 | 26.40 | B |
| ATOM | 2882 | N | VAL | 52 | 46.998 | −13.213 | 61.908 | 1.00 | 27.16 | B |
| ATOM | 2883 | CA | VAL | 52 | 48.000 | −12.463 | 62.657 | 1.00 | 29.10 | B |
| ATOM | 2884 | CB | VAL | 52 | 47.311 | −11.480 | 63.640 | 1.00 | 28.02 | B |
| ATOM | 2885 | CG1 | VAL | 52 | 48.336 | −10.624 | 64.340 | 1.00 | 27.20 | B |
| ATOM | 2886 | CG2 | VAL | 52 | 46.341 | −10.607 | 62.885 | 1.00 | 27.34 | B |
| ATOM | 2887 | C | VAL | 52 | 48.974 | −13.331 | 63.442 | 1.00 | 30.28 | B |
| ATOM | 2888 | O | VAL | 52 | 48.567 | −14.267 | 64.117 | 1.00 | 30.72 | B |
| ATOM | 2889 | N | ARG | 53 | 50.265 | −13.018 | 63.342 | 1.00 | 31.46 | B |
| ATOM | 2890 | CA | ARG | 53 | 51.276 | −13.778 | 64.070 | 1.00 | 32.95 | B |
| ATOM | 2891 | CB | ARG | 53 | 52.615 | −13.750 | 63.336 | 1.00 | 33.14 | B |
| ATOM | 2892 | CG | ARG | 53 | 53.636 | −14.706 | 63.926 | 1.00 | 32.63 | B |
| ATOM | 2893 | CD | ARG | 53 | 54.575 | −15.197 | 62.851 | 1.00 | 33.53 | B |
| ATOM | 2894 | NE | ARG | 53 | 55.482 | −14.163 | 62.378 | 1.00 | 34.35 | B |
| ATOM | 2895 | CZ | ARG | 53 | 56.017 | −14.140 | 61.161 | 1.00 | 35.36 | B |
| ATOM | 2896 | NH1 | ARG | 53 | 55.738 | −15.089 | 60.272 | 1.00 | 35.11 | B |
| ATOM | 2897 | NH2 | ARG | 53 | 56.847 | −13.162 | 60.838 | 1.00 | 36.70 | B |
| ATOM | 2898 | C | ARG | 53 | 51.423 | −13.182 | 65.458 | 1.00 | 34.27 | B |
| ATOM | 2899 | O | ARG | 53 | 51.964 | −12.088 | 65.632 | 1.00 | 34.80 | B |
| ATOM | 2900 | N | THR | 54 | 50.931 | −13.915 | 66.446 | 1.00 | 35.04 | B |
| ATOM | 2901 | CA | THR | 54 | 50.977 | −13.458 | 67.815 | 1.00 | 37.72 | B |
| ATOM | 2902 | CB | THR | 54 | 49.672 | −13.823 | 68.540 | 1.00 | 37.47 | B |
| ATOM | 2903 | OG1 | THR | 54 | 49.521 | −15.244 | 68.581 | 1.00 | 36.02 | B |
| ATOM | 2904 | CG2 | THR | 54 | 48.484 | −13.260 | 67.804 | 1.00 | 37.61 | B |
| ATOM | 2905 | C | THR | 54 | 52.141 | −14.056 | 68.586 | 1.00 | 39.85 | B |
| ATOM | 2906 | O | THR | 54 | 52.517 | −13.554 | 69.633 | 1.00 | 39.10 | B |
| ATOM | 2907 | N | GLY | 55 | 52.721 | −15.121 | 68.043 | 1.00 | 43.17 | B |
| ATOM | 2908 | CA | GLY | 55 | 53.810 | −15.791 | 68.727 | 1.00 | 48.23 | B |
| ATOM | 2909 | C | GLY | 55 | 55.214 | −15.667 | 68.165 | 1.00 | 51.61 | B |
| ATOM | 2910 | O | GLY | 55 | 55.704 | −14.562 | 67.926 | 1.00 | 52.45 | B |
| ATOM | 2911 | N | GLY | 56 | 55.855 | −16.820 | 67.962 | 1.00 | 53.22 | B |
| ATOM | 2912 | CA | GLY | 56 | 57.219 | −16.864 | 67.464 | 1.00 | 54.95 | B |
| ATOM | 2913 | C | GLY | 56 | 57.420 | −16.365 | 66.052 | 1.00 | 56.66 | B |
| ATOM | 2914 | O | GLY | 56 | 56.733 | −15.450 | 65.611 | 1.00 | 57.44 | B |
| ATOM | 2915 | N | LEU | 57 | 58.366 | −16.980 | 65.346 | 1.00 | 57.72 | B |
| ATOM | 2916 | CA | LEU | 57 | 58.693 | −16.600 | 63.972 | 1.00 | 58.30 | B |
| ATOM | 2917 | CB | LEU | 57 | 60.219 | −16.608 | 63.777 | 1.00 | 58.78 | B |
| ATOM | 2918 | CG | LEU | 57 | 61.067 | −17.384 | 64.790 | 1.00 | 59.20 | B |
| ATOM | 2919 | CD1 | LEU | 57 | 60.709 | −18.870 | 64.762 | 1.00 | 59.75 | B |
| ATOM | 2920 | CD2 | LEU | 57 | 62.542 | −17.175 | 64.472 | 1.00 | 59.20 | B |
| ATOM | 2921 | C | LEU | 57 | 58.029 | −17.493 | 62.921 | 1.00 | 58.10 | B |
| ATOM | 2922 | O | LEU | 57 | 57.153 | −18.289 | 63.245 | 1.00 | 58.57 | B |
| ATOM | 2923 | N | ALA | 58 | 58.450 | −17.343 | 61.665 | 1.00 | 57.02 | B |
| ATOM | 2924 | CA | ALA | 58 | 57.905 | −18.126 | 60.555 | 1.00 | 55.81 | B |
| ATOM | 2925 | CB | ALA | 58 | 58.473 | −17.615 | 59.235 | 1.00 | 55.75 | B |
| ATOM | 2926 | C | ALA | 58 | 58.193 | −19.622 | 60.705 | 1.00 | 54.88 | B |
| ATOM | 2927 | O | ALA | 58 | 57.350 | −20.460 | 60.375 | 1.00 | 54.40 | B |
| ATOM | 2928 | N | ASP | 59 | 59.386 | −19.937 | 61.211 | 1.00 | 53.60 | B |
| ATOM | 2929 | CA | ASP | 59 | 59.845 | −21.316 | 61.431 | 1.00 | 51.49 | B |
| ATOM | 2930 | CB | ASP | 59 | 61.254 | −21.290 | 62.050 | 1.00 | 51.99 | B |
| ATOM | 2931 | CG | ASP | 59 | 61.807 | −22.681 | 62.338 | 1.00 | 52.10 | B |
| ATOM | 2932 | OD1 | ASP | 59 | 62.005 | −23.464 | 61.385 | 1.00 | 51.56 | B |
| ATOM | 2933 | OD2 | ASP | 59 | 62.051 | −22.987 | 63.525 | 1.00 | 52.60 | B |
| ATOM | 2934 | C | ASP | 59 | 58.903 | −22.110 | 62.338 | 1.00 | 49.40 | B |
| ATOM | 2935 | O | ASP | 59 | 58.742 | −23.315 | 62.197 | 1.00 | 48.84 | B |
| ATOM | 2936 | N | LYS | 60 | 58.267 | −21.404 | 63.256 | 1.00 | 47.59 | B |
| ATOM | 2937 | CA | LYS | 60 | 57.366 | −22.021 | 64.208 | 1.00 | 46.47 | B |
| ATOM | 2938 | CB | LYS | 60 | 58.178 | −22.949 | 65.114 | 1.00 | 45.88 | B |
| ATOM | 2939 | CG | LYS | 60 | 57.465 | −23.470 | 66.345 | 1.00 | 44.88 | B |
| ATOM | 2940 | CD | LYS | 60 | 58.462 | −24.217 | 67.209 | 1.00 | 45.79 | B |
| ATOM | 2941 | CE | LYS | 60 | 57.868 | −24.729 | 68.503 | 1.00 | 47.18 | B |
| ATOM | 2942 | NZ | LYS | 60 | 58.938 | −25.298 | 69.384 | 1.00 | 48.54 | B |
| ATOM | 2943 | C | LYS | 60 | 56.745 | −20.862 | 64.977 | 1.00 | 45.74 | B |
| ATOM | 2944 | O | LYS | 60 | 57.468 | −20.017 | 65.532 | 1.00 | 45.66 | B |
| ATOM | 2945 | N | SER | 61 | 55.417 | −20.802 | 64.999 | 1.00 | 44.14 | B |
| ATOM | 2946 | CA | SER | 61 | 54.750 | −19.718 | 65.697 | 1.00 | 42.32 | B |
| ATOM | 2947 | CB | SER | 61 | 54.900 | −18.419 | 64.892 | 1.00 | 43.45 | B |
| ATOM | 2948 | OG | SER | 61 | 54.484 | −18.594 | 63.545 | 1.00 | 42.02 | B |
| ATOM | 2949 | C | SER | 61 | 53.267 | −19.931 | 65.980 | 1.00 | 40.98 | B |
| ATOM | 2950 | O | SER | 61 | 52.679 | −20.939 | 65.613 | 1.00 | 40.30 | B |
| ATOM | 2951 | N | SER | 62 | 52.686 | −18.954 | 66.669 | 1.00 | 40.63 | B |
| ATOM | 2952 | CA | SER | 62 | 51.265 | −18.944 | 66.992 | 1.00 | 38.79 | B |
| ATOM | 2953 | CB | SER | 62 | 51.032 | −18.549 | 68.445 | 1.00 | 38.80 | B |
| ATOM | 2954 | OG | SER | 62 | 51.678 | −19.441 | 69.325 | 1.00 | 38.30 | B |
| ATOM | 2955 | C | SER | 62 | 50.634 | −17.862 | 66.115 | 1.00 | 37.30 | B |
| ATOM | 2956 | O | SER | 62 | 51.293 | −16.906 | 65.728 | 1.00 | 37.14 | B |
| ATOM | 2957 | N | ARG | 63 | 49.361 | −18.018 | 65.783 | 1.00 | 36.69 | B |
| ATOM | 2958 | CA | ARG | 63 | 48.687 | −17.017 | 64.959 | 1.00 | 35.86 | B |
| ATOM | 2959 | CB | ARG | 63 | 48.827 | −17.318 | 63.453 | 1.00 | 35.76 | B |
| ATOM | 2960 | CG | ARG | 63 | 50.264 | −17.378 | 62.918 | 1.00 | 36.93 | B |
| ATOM | 2961 | CD | ARG | 63 | 50.303 | −17.660 | 61.418 | 1.00 | 38.47 | B |
| ATOM | 2962 | NE | ARG | 63 | 49.917 | −16.499 | 60.608 | 1.00 | 40.73 | B |
| ATOM | 2963 | CZ | ARG | 63 | 50.685 | −15.428 | 60.393 | 1.00 | 40.83 | B |
| ATOM | 2964 | NH1 | ARG | 63 | 51.896 | −15.353 | 60.928 | 1.00 | 41.75 | B |
| ATOM | 2965 | NH2 | ARG | 63 | 50.250 | −14.433 | 59.629 | 1.00 | 40.58 | B |
| ATOM | 2966 | C | ARG | 63 | 47.206 | −16.982 | 65.296 | 1.00 | 34.60 | B |
| ATOM | 2967 | O | ARG | 63 | 46.656 | −17.920 | 65.855 | 1.00 | 33.92 | B |
| ATOM | 2968 | N | LYS | 64 | 46.578 | −15.865 | 64.968 | 1.00 | 33.48 | B |
| ATOM | 2969 | CA | LYS | 64 | 45.158 | −15.676 | 65.193 | 1.00 | 31.00 | B |
| ATOM | 2970 | CB | LYS | 64 | 44.913 | −14.444 | 66.056 | 1.00 | 34.47 | B |
| ATOM | 2971 | CG | LYS | 64 | 45.324 | −14.581 | 67.508 | 1.00 | 36.74 | B |
| ATOM | 2972 | CD | LYS | 64 | 44.298 | −15.378 | 68.279 | 1.00 | 38.57 | B |
| ATOM | 2973 | CE | LYS | 64 | 44.593 | −15.324 | 69.773 | 1.00 | 39.71 | B |
| ATOM | 2974 | NZ | LYS | 64 | 43.520 | −15.964 | 70.596 | 1.00 | 40.02 | B |
| ATOM | 2975 | C | LYS | 64 | 44.592 | −15.428 | 63.805 | 1.00 | 29.35 | B |
| ATOM | 2976 | O | LYS | 64 | 45.114 | −14.604 | 63.045 | 1.00 | 29.23 | B |
| ATOM | 2977 | N | THR | 65 | 43.537 | −16.156 | 63.470 | 1.00 | 27.29 | B |
| ATOM | 2978 | CA | THR | 65 | 42.917 | −16.020 | 62.165 | 1.00 | 24.96 | B |
| ATOM | 2979 | CB | THR | 65 | 43.062 | −17.321 | 61.338 | 1.00 | 24.86 | B |
| ATOM | 2980 | OG1 | THR | 65 | 44.442 | −17.701 | 61.294 | 1.00 | 24.93 | B |
| ATOM | 2981 | CG2 | THR | 65 | 42.555 | −17.120 | 59.912 | 1.00 | 25.70 | B |
| ATOM | 2982 | C | THR | 65 | 41.449 | −15.688 | 62.319 | 1.00 | 22.74 | B |
| ATOM | 2983 | O | THR | 65 | 40.752 | −16.313 | 63.095 | 1.00 | 23.83 | B |
| ATOM | 2984 | N | TYR | 66 | 40.999 | −14.677 | 61.579 | 1.00 | 21.85 | B |
| ATOM | 2985 | CA | TYR | 66 | 39.601 | −14.232 | 61.612 | 1.00 | 20.45 | B |
| ATOM | 2986 | CB | TYR | 66 | 39.480 | −12.844 | 62.234 | 1.00 | 18.74 | B |
| ATOM | 2987 | CG | TYR | 66 | 40.144 | −12.695 | 63.581 | 1.00 | 19.02 | B |
| ATOM | 2988 | CD1 | TYR | 66 | 41.524 | −12.584 | 63.695 | 1.00 | 18.23 | B |
| ATOM | 2989 | CE1 | TYR | 66 | 42.136 | −12.420 | 64.946 | 1.00 | 19.22 | B |
| ATOM | 2990 | CD2 | TYR | 66 | 39.387 | −12.641 | 64.748 | 1.00 | 20.12 | B |
| ATOM | 2991 | CE2 | TYR | 66 | 39.986 | −12.474 | 66.009 | 1.00 | 19.66 | B |
| ATOM | 2992 | CZ | TYR | 66 | 41.357 | −12.367 | 66.109 | 1.00 | 20.40 | B |
| ATOM | 2993 | OH | TYR | 66 | 41.915 | −12.234 | 67.382 | 1.00 | 20.35 | B |
| ATOM | 2994 | C | TYR | 66 | 39.027 | −14.136 | 60.195 | 1.00 | 22.62 | B |
| ATOM | 2995 | O | TYR | 66 | 39.736 | −13.786 | 59.237 | 1.00 | 22.83 | B |
| ATOM | 2996 | N | THR | 67 | 37.747 | −14.464 | 60.058 | 1.00 | 22.62 | B |
| ATOM | 2997 | CA | THR | 67 | 37.099 | −14.424 | 58.755 | 1.00 | 23.36 | B |
| ATOM | 2998 | CB | THR | 67 | 36.299 | −15.723 | 58.489 | 1.00 | 24.24 | B |
| ATOM | 2999 | OG1 | THR | 67 | 37.169 | −16.854 | 58.576 | 1.00 | 26.83 | B |
| ATOM | 3000 | CG2 | THR | 67 | 35.679 | −15.702 | 57.115 | 1.00 | 25.09 | B |
| ATOM | 3001 | C | THR | 67 | 36.145 | −13.241 | 58.669 | 1.00 | 23.25 | B |
| ATOM | 3002 | O | THR | 67 | 35.383 | −12.979 | 59.598 | 1.00 | 23.74 | B |
| ATOM | 3003 | N | PHE | 68 | 36.199 | −12.521 | 57.556 | 1.00 | 22.27 | B |
| ATOM | 3004 | CA | PHE | 68 | 35.322 | −11.379 | 57.354 | 1.00 | 23.47 | B |
| ATOM | 3005 | CB | PHE | 68 | 36.108 | −10.068 | 57.414 | 1.00 | 25.18 | B |
| ATOM | 3006 | CG | PHE | 68 | 36.688 | −9.788 | 58.758 | 1.00 | 28.91 | B |
| ATOM | 3007 | CD1 | PHE | 68 | 37.872 | −10.407 | 59.162 | 1.00 | 31.76 | B |
| ATOM | 3008 | CD2 | PHE | 68 | 36.028 | −8.957 | 59.655 | 1.00 | 30.45 | B |
| ATOM | 3009 | CE1 | PHE | 68 | 38.397 | −10.211 | 60.444 | 1.00 | 33.13 | B |
| ATOM | 3010 | CE2 | PHE | 68 | 36.539 | −8.749 | 60.947 | 1.00 | 32.68 | B |
| ATOM | 3011 | CZ | PHE | 68 | 37.733 | −9.381 | 61.346 | 1.00 | 34.40 | B |
| ATOM | 3012 | C | PHE | 68 | 34.664 | −11.530 | 56.001 | 1.00 | 23.18 | B |
| ATOM | 3013 | O | PHE | 68 | 34.904 | −12.505 | 55.318 | 1.00 | 23.09 | B |
| ATOM | 3014 | N | ASP | 69 | 33.836 | −10.560 | 55.625 | 1.00 | 22.35 | B |
| ATOM | 3015 | CA | ASP | 69 | 33.127 | −10.585 | 54.350 | 1.00 | 23.38 | B |
| ATOM | 3016 | CB | ASP | 69 | 31.988 | −9.559 | 54.386 | 1.00 | 23.05 | B |
| ATOM | 3017 | CG | ASP | 69 | 30.917 | −9.915 | 55.427 | 1.00 | 23.94 | B |
| ATOM | 3018 | OD1 | ASP | 69 | 30.875 | −9.341 | 56.538 | 1.00 | 21.68 | B |
| ATOM | 3019 | OD2 | ASP | 69 | 30.106 | −10.812 | 55.138 | 1.00 | 25.46 | B |
| ATOM | 3020 | C | ASP | 69 | 34.071 | −10.363 | 53.173 | 1.00 | 24.90 | B |
| ATOM | 3021 | O | ASP | 69 | 33.880 | −10.931 | 52.082 | 1.00 | 25.83 | B |
| ATOM | 3022 | N | MET | 70 | 35.089 | −9.539 | 53.405 | 1.00 | 25.78 | B |
| ATOM | 3023 | CA | MET | 70 | 36.112 | −9.233 | 52.412 | 1.00 | 26.18 | B |
| ATOM | 3024 | CB | MET | 70 | 35.686 | −8.073 | 51.517 | 1.00 | 27.89 | B |
| ATOM | 3025 | CG | MET | 70 | 34.538 | −8.363 | 50.564 | 1.00 | 29.68 | B |
| ATOM | 3026 | SD | MET | 70 | 34.155 | −6.927 | 49.495 | 1.00 | 34.95 | B |
| ATOM | 3027 | CE | MET | 70 | 32.418 | −7.227 | 49.126 | 1.00 | 32.58 | B |
| ATOM | 3028 | C | MET | 70 | 37.378 | −8.801 | 53.150 | 1.00 | 25.52 | B |
| ATOM | 3029 | O | MET | 70 | 37.301 | −8.187 | 54.206 | 1.00 | 26.04 | B |
| ATOM | 3030 | N | VAL | 71 | 38.540 | −9.119 | 52.596 | 1.00 | 24.01 | B |
| ATOM | 3031 | CA | VAL | 71 | 39.789 | −8.724 | 53.228 | 1.00 | 23.48 | B |
| ATOM | 3032 | CB | VAL | 71 | 40.496 | −9.917 | 53.902 | 1.00 | 24.24 | B |
| ATOM | 3033 | CG1 | VAL | 71 | 39.668 | −10.429 | 55.086 | 1.00 | 23.32 | B |
| ATOM | 3034 | CG2 | VAL | 71 | 40.726 | −11.004 | 52.882 | 1.00 | 24.53 | B |
| ATOM | 3035 | C | VAL | 71 | 40.709 | −8.121 | 52.181 | 1.00 | 23.86 | B |
| ATOM | 3036 | O | VAL | 71 | 40.841 | −8.641 | 51.068 | 1.00 | 22.79 | B |
| ATOM | 3037 | N | PHE | 72 | 41.356 | −7.025 | 52.551 | 1.00 | 22.62 | B |
| ATOM | 3038 | CA | PHE | 72 | 42.229 | −6.344 | 51.628 | 1.00 | 22.70 | B |
| ATOM | 3039 | CB | PHE | 72 | 41.710 | −4.936 | 51.321 | 1.00 | 20.63 | B |
| ATOM | 3040 | CG | PHE | 72 | 40.318 | −4.910 | 50.753 | 1.00 | 18.35 | B |
| ATOM | 3041 | CD1 | PHE | 72 | 40.056 | −5.419 | 49.493 | 1.00 | 15.95 | B |
| ATOM | 3042 | CD2 | PHE | 72 | 39.261 | −4.409 | 51.495 | 1.00 | 17.50 | B |
| ATOM | 3043 | CE1 | PHE | 72 | 38.771 | −5.435 | 48.986 | 1.00 | 16.14 | B |
| ATOM | 3044 | CE2 | PHE | 72 | 37.976 | −4.425 | 50.985 | 1.00 | 17.48 | B |
| ATOM | 3045 | CZ | PHE | 72 | 37.732 | −4.939 | 49.729 | 1.00 | 16.21 | B |
| ATOM | 3046 | C | PHE | 72 | 43.626 | −6.197 | 52.178 | 1.00 | 22.69 | B |
| ATOM | 3047 | O | PHE | 72 | 43.836 | −5.523 | 53.181 | 1.00 | 22.50 | B |
| ATOM | 3048 | N | GLY | 73 | 44.578 | −6.837 | 51.508 | 1.00 | 22.82 | B |
| ATOM | 3049 | CA | GLY | 73 | 45.965 | −6.741 | 51.920 | 1.00 | 23.34 | B |
| ATOM | 3050 | C | GLY | 73 | 46.584 | −5.398 | 51.571 | 1.00 | 23.29 | B |
| ATOM | 3051 | O | GLY | 73 | 45.982 | −4.561 | 50.885 | 1.00 | 22.64 | B |
| ATOM | 3052 | N | ALA | 74 | 47.809 | −5.199 | 52.037 | 1.00 | 23.40 | B |
| ATOM | 3053 | CA | ALA | 74 | 48.531 | −3.960 | 51.808 | 1.00 | 25.70 | B |
| ATOM | 3054 | CB | ALA | 74 | 49.891 | −4.016 | 52.523 | 1.00 | 25.78 | B |
| ATOM | 3055 | C | ALA | 74 | 48.725 | −3.639 | 50.328 | 1.00 | 26.16 | B |
| ATOM | 3056 | O | ALA | 74 | 49.129 | −2.556 | 49.978 | 1.00 | 27.50 | B |
| ATOM | 3057 | N | SER | 75 | 48.406 | −4.584 | 49.459 | 1.00 | 27.00 | B |
| ATOM | 3058 | CA | SER | 75 | 48.590 | −4.358 | 48.031 | 1.00 | 28.47 | B |
| ATOM | 3059 | CB | SER | 75 | 48.982 | −5.679 | 47.335 | 1.00 | 28.85 | B |
| ATOM | 3060 | OG | SER | 75 | 48.019 | −6.709 | 47.507 | 1.00 | 27.19 | B |
| ATOM | 3061 | C | SER | 75 | 47.389 | −3.728 | 47.319 | 1.00 | 27.90 | B |
| ATOM | 3062 | O | SER | 75 | 47.542 | −3.123 | 46.243 | 1.00 | 29.21 | B |
| ATOM | 3063 | N | THR | 76 | 46.206 | −3.853 | 47.918 | 1.00 | 26.99 | B |
| ATOM | 3064 | CA | THR | 76 | 44.984 | −3.315 | 47.320 | 1.00 | 25.45 | B |
| ATOM | 3065 | CB | THR | 76 | 43.746 | −3.663 | 48.183 | 1.00 | 23.54 | B |
| ATOM | 3066 | OG1 | THR | 76 | 44.015 | −3.345 | 49.545 | 1.00 | 23.44 | B |
| ATOM | 3067 | CG2 | THR | 76 | 43.436 | −5.132 | 48.116 | 1.00 | 24.38 | B |
| ATOM | 3068 | C | THR | 76 | 45.034 | −1.803 | 47.087 | 1.00 | 25.69 | B |
| ATOM | 3069 | O | THR | 76 | 45.543 | −1.041 | 47.922 | 1.00 | 27.74 | B |
| ATOM | 3070 | N | LYS | 77 | 44.507 | −1.372 | 45.948 | 1.00 | 24.67 | B |
| ATOM | 3071 | CA | LYS | 77 | 44.496 | 0.044 | 45.619 | 1.00 | 23.51 | B |
| ATOM | 3072 | CB | LYS | 77 | 44.804 | 0.234 | 44.133 | 1.00 | 25.56 | B |
| ATOM | 3073 | CG | LYS | 77 | 46.192 | −0.249 | 43.719 | 1.00 | 28.23 | B |
| ATOM | 3074 | CD | LYS | 77 | 46.373 | −0.132 | 42.209 | 1.00 | 31.78 | B |
| ATOM | 3075 | CE | LYS | 77 | 47.770 | −0.560 | 41.784 | 1.00 | 33.69 | B |
| ATOM | 3076 | NZ | LYS | 77 | 47.942 | −0.449 | 40.311 | 1.00 | 35.35 | B |
| ATOM | 3077 | C | LYS | 77 | 43.150 | 0.677 | 45.956 | 1.00 | 21.23 | B |
| ATOM | 3078 | O | LYS | 77 | 42.175 | −0.023 | 46.154 | 1.00 | 19.65 | B |
| ATOM | 3079 | N | GLN | 78 | 43.105 | 2.008 | 46.021 | 1.00 | 20.16 | B |
| ATOM | 3080 | CA | GLN | 78 | 41.853 | 2.714 | 46.335 | 1.00 | 18.91 | B |
| ATOM | 3081 | CB | GLN | 78 | 42.004 | 4.226 | 46.179 | 1.00 | 18.69 | B |
| ATOM | 3082 | CG | GLN | 78 | 43.063 | 4.851 | 47.064 | 1.00 | 18.42 | B |
| ATOM | 3083 | CD | GLN | 78 | 42.618 | 4.962 | 48.498 | 1.00 | 17.41 | B |
| ATOM | 3084 | OE1 | GLN | 78 | 42.152 | 3.997 | 49.085 | 1.00 | 20.11 | B |
| ATOM | 3085 | NE2 | GLN | 78 | 42.756 | 6.143 | 49.066 | 1.00 | 14.62 | B |
| ATOM | 3086 | C | GLN | 78 | 40.743 | 2.294 | 45.377 | 1.00 | 19.40 | B |
| ATOM | 3087 | O | GLN | 78 | 39.609 | 2.059 | 45.788 | 1.00 | 20.13 | B |
| ATOM | 3088 | N | ILE | 79 | 41.074 | 2.208 | 44.092 | 1.00 | 17.68 | B |
| ATOM | 3089 | CA | ILE | 79 | 40.089 | 1.815 | 43.094 | 1.00 | 15.86 | B |
| ATOM | 3090 | CB | ILE | 79 | 40.727 | 1.779 | 41.678 | 1.00 | 15.34 | B |
| ATOM | 3091 | CG2 | ILE | 79 | 41.709 | 0.597 | 41.561 | 1.00 | 16.93 | B |
| ATOM | 3092 | CG1 | ILE | 79 | 39.640 | 1.641 | 40.612 | 1.00 | 14.82 | B |
| ATOM | 3093 | CD1 | ILE | 79 | 38.766 | 2.868 | 40.410 | 1.00 | 13.32 | B |
| ATOM | 3094 | C | ILE | 79 | 39.463 | 0.440 | 43.399 | 1.00 | 14.58 | B |
| ATOM | 3095 | O | ILE | 79 | 38.304 | 0.217 | 43.130 | 1.00 | 15.24 | B |
| ATOM | 3096 | N | ASP | 80 | 40.231 | −0.479 | 43.969 | 1.00 | 13.09 | B |
| ATOM | 3097 | CA | ASP | 80 | 39.683 | −1.802 | 44.258 | 1.00 | 12.77 | B |
| ATOM | 3098 | CB | ASP | 80 | 40.800 | −2.818 | 44.435 | 1.00 | 14.43 | B |
| ATOM | 3099 | CG | ASP | 80 | 41.645 | −2.953 | 43.204 | 1.00 | 18.24 | B |
| ATOM | 3100 | OD1 | ASP | 80 | 41.072 | −2.882 | 42.088 | 1.00 | 18.91 | B |
| ATOM | 3101 | OD2 | ASP | 80 | 42.874 | −3.140 | 43.363 | 1.00 | 21.75 | B |
| ATOM | 3102 | C | ASP | 80 | 38.787 | −1.829 | 45.487 | 1.00 | 12.00 | B |
| ATOM | 3103 | O | ASP | 80 | 37.878 | −2.638 | 45.590 | 1.00 | 10.17 | B |
| ATOM | 3104 | N | VAL | 81 | 39.063 | −0.938 | 46.430 | 1.00 | 11.87 | B |
| ATOM | 3105 | CA | VAL | 81 | 38.261 | −0.841 | 47.638 | 1.00 | 10.20 | B |
| ATOM | 3106 | CB | VAL | 81 | 38.881 | 0.128 | 48.642 | 1.00 | 9.09 | B |
| ATOM | 3107 | CG1 | VAL | 81 | 37.857 | 0.529 | 49.689 | 1.00 | 7.52 | B |
| ATOM | 3108 | CG2 | VAL | 81 | 40.071 | −0.534 | 49.299 | 1.00 | 11.81 | B |
| ATOM | 3109 | C | VAL | 81 | 36.915 | −0.292 | 47.224 | 1.00 | 10.85 | B |
| ATOM | 3110 | O | VAL | 81 | 35.879 | −0.728 | 47.697 | 1.00 | 11.76 | B |
| ATOM | 3111 | N | TYR | 82 | 36.948 | 0.681 | 46.326 | 1.00 | 12.12 | B |
| ATOM | 3112 | CA | TYR | 82 | 35.735 | 1.304 | 45.845 | 1.00 | 13.85 | B |
| ATOM | 3113 | CB | TYR | 82 | 36.090 | 2.534 | 45.015 | 1.00 | 15.89 | B |
| ATOM | 3114 | CG | TYR | 82 | 34.870 | 3.259 | 44.530 | 1.00 | 18.66 | B |
| ATOM | 3115 | CD1 | TYR | 82 | 34.364 | 3.029 | 43.256 | 1.00 | 20.38 | B |
| ATOM | 3116 | CE1 | TYR | 82 | 33.201 | 3.645 | 42.824 | 1.00 | 22.59 | B |
| ATOM | 3117 | CD2 | TYR | 82 | 34.184 | 4.132 | 45.369 | 1.00 | 19.71 | B |
| ATOM | 3118 | CE2 | TYR | 82 | 33.019 | 4.755 | 44.953 | 1.00 | 22.44 | B |
| ATOM | 3119 | CZ | TYR | 82 | 32.531 | 4.508 | 43.675 | 1.00 | 23.44 | B |
| ATOM | 3120 | OH | TYR | 82 | 31.372 | 5.125 | 43.254 | 1.00 | 25.79 | B |
| ATOM | 3121 | C | TYR | 82 | 34.840 | 0.350 | 45.044 | 1.00 | 14.77 | B |
| ATOM | 3122 | O | TYR | 82 | 33.635 | 0.211 | 45.331 | 1.00 | 13.77 | B |
| ATOM | 3123 | N | ARG | 83 | 35.408 | −0.299 | 44.035 | 1.00 | 15.58 | B |
| ATOM | 3124 | CA | ARG | 83 | 34.632 | −1.236 | 43.220 | 1.00 | 18.14 | B |
| ATOM | 3125 | CB | ARG | 83 | 35.517 | −1.815 | 42.103 | 1.00 | 20.58 | B |
| ATOM | 3126 | CG | ARG | 83 | 35.715 | −0.868 | 40.915 | 1.00 | 23.85 | B |
| ATOM | 3127 | CD | ARG | 83 | 36.998 | −1.162 | 40.161 | 1.00 | 26.52 | B |
| ATOM | 3128 | NE | ARG | 83 | 36.971 | −2.428 | 39.436 | 1.00 | 30.77 | B |
| ATOM | 3129 | CZ | ARG | 83 | 36.255 | −2.656 | 38.335 | 1.00 | 33.35 | B |
| ATOM | 3130 | NH1 | ARG | 83 | 35.485 | −1.703 | 37.818 | 1.00 | 33.79 | B |
| ATOM | 3131 | NH2 | ARG | 83 | 36.339 | −3.833 | 37.727 | 1.00 | 33.17 | B |
| ATOM | 3132 | C | ARG | 83 | 34.009 | −2.382 | 44.045 | 1.00 | 18.55 | B |
| ATOM | 3133 | O | ARG | 83 | 32.867 | −2.765 | 43.834 | 1.00 | 19.46 | B |
| ATOM | 3134 | N | SER | 84 | 34.764 | −2.930 | 44.985 | 1.00 | 17.88 | B |
| ATOM | 3135 | CA | SER | 84 | 34.248 | −4.009 | 45.809 | 1.00 | 17.71 | B |
| ATOM | 3136 | CB | SER | 84 | 35.380 | −4.764 | 46.509 | 1.00 | 20.38 | B |
| ATOM | 3137 | OG | SER | 84 | 36.282 | −5.324 | 45.575 | 1.00 | 25.36 | B |
| ATOM | 3138 | C | SER | 84 | 33.298 | −3.551 | 46.913 | 1.00 | 16.07 | B |
| ATOM | 3139 | O | SER | 84 | 32.241 | −4.113 | 47.073 | 1.00 | 15.35 | B |
| ATOM | 3140 | N | VAL | 85 | 33.685 | −2.526 | 47.673 | 1.00 | 15.30 | B |
| ATOM | 3141 | CA | VAL | 85 | 32.865 | −2.048 | 48.795 | 1.00 | 14.98 | B |
| ATOM | 3142 | CB | VAL | 85 | 33.738 | −1.521 | 49.963 | 1.00 | 15.00 | B |
| ATOM | 3143 | CG1 | VAL | 85 | 32.849 | −1.183 | 51.129 | 1.00 | 15.00 | B |
| ATOM | 3144 | CG2 | VAL | 85 | 34.775 | −2.556 | 50.383 | 1.00 | 15.18 | B |
| ATOM | 3145 | C | VAL | 85 | 31.828 | −0.960 | 48.509 | 1.00 | 14.85 | B |
| ATOM | 3146 | O | VAL | 85 | 30.652 | −1.162 | 48.734 | 1.00 | 13.96 | B |
| ATOM | 3147 | N | VAL | 86 | 32.283 | 0.184 | 48.008 | 1.00 | 16.21 | B |
| ATOM | 3148 | CA | VAL | 86 | 31.409 | 1.313 | 47.740 | 1.00 | 15.47 | B |
| ATOM | 3149 | CB | VAL | 86 | 32.205 | 2.597 | 47.571 | 1.00 | 15.27 | B |
| ATOM | 3150 | CG1 | VAL | 86 | 31.296 | 3.776 | 47.800 | 1.00 | 15.63 | B |
| ATOM | 3151 | CG2 | VAL | 86 | 33.379 | 2.614 | 48.541 | 1.00 | 16.09 | B |
| ATOM | 3152 | C | VAL | 86 | 30.478 | 1.191 | 46.548 | 1.00 | 15.77 | B |
| ATOM | 3153 | O | VAL | 86 | 29.295 | 1.506 | 46.680 | 1.00 | 15.71 | B |
| ATOM | 3154 | N | CYS | 87 | 30.976 | 0.734 | 45.399 | 1.00 | 15.31 | B |
| ATOM | 3155 | CA | CYS | 87 | 30.121 | 0.629 | 44.218 | 1.00 | 17.14 | B |
| ATOM | 3156 | CB | CYS | 87 | 30.787 | −0.168 | 43.108 | 1.00 | 16.23 | B |
| ATOM | 3157 | SG | CYS | 87 | 30.003 | 0.173 | 41.511 | 1.00 | 22.71 | B |
| ATOM | 3158 | C | CYS | 87 | 28.753 | −0.001 | 44.488 | 1.00 | 18.54 | B |
| ATOM | 3159 | O | CYS | 87 | 27.752 | 0.494 | 44.050 | 1.00 | 19.06 | B |
| ATOM | 3160 | N | PRO | 88 | 28.707 | −1.117 | 45.207 | 1.00 | 20.44 | B |
| ATOM | 3161 | CD | PRO | 88 | 29.827 | −2.005 | 45.536 | 1.00 | 22.48 | B |
| ATOM | 3162 | CA | PRO | 88 | 27.422 | −1.759 | 45.507 | 1.00 | 21.26 | B |
| ATOM | 3163 | CB | PRO | 88 | 27.847 | −3.060 | 46.157 | 1.00 | 21.76 | B |
| ATOM | 3164 | CG | PRO | 88 | 29.168 | −3.337 | 45.512 | 1.00 | 22.69 | B |
| ATOM | 3165 | C | PRO | 88 | 26.542 | −0.890 | 46.434 | 1.00 | 22.59 | B |
| ATOM | 3166 | O | PRO | 88 | 25.333 | −0.797 | 46.254 | 1.00 | 22.78 | B |
| ATOM | 3167 | N | ILE | 89 | 27.151 | −0.273 | 47.446 | 1.00 | 22.51 | B |
| ATOM | 3168 | CA | ILE | 89 | 26.409 | 0.582 | 48.388 | 1.00 | 22.44 | B |
| ATOM | 3169 | CB | ILE | 89 | 27.298 | 1.003 | 49.579 | 1.00 | 22.87 | B |
| ATOM | 3170 | CG2 | ILE | 89 | 26.592 | 2.040 | 50.408 | 1.00 | 22.27 | B |
| ATOM | 3171 | CG1 | ILE | 89 | 27.607 | −0.227 | 50.439 | 1.00 | 24.48 | B |
| ATOM | 3172 | CD1 | ILE | 89 | 28.465 | 0.041 | 51.641 | 1.00 | 26.67 | B |
| ATOM | 3173 | C | ILE | 89 | 25.843 | 1.841 | 47.727 | 1.00 | 22.09 | B |
| ATOM | 3174 | O | ILE | 89 | 24.734 | 2.264 | 48.035 | 1.00 | 21.69 | B |
| ATOM | 3175 | N | LEU | 90 | 26.607 | 2.450 | 46.829 | 1.00 | 21.87 | B |
| ATOM | 3176 | CA | LEU | 90 | 26.122 | 3.640 | 46.157 | 1.00 | 23.17 | B |
| ATOM | 3177 | CB | LEU | 90 | 27.195 | 4.228 | 45.243 | 1.00 | 20.80 | B |
| ATOM | 3178 | CG | LEU | 90 | 26.773 | 5.485 | 44.498 | 1.00 | 18.97 | B |
| ATOM | 3179 | CD1 | LEU | 90 | 26.169 | 6.492 | 45.446 | 1.00 | 18.16 | B |
| ATOM | 3180 | CD2 | LEU | 90 | 27.987 | 6.053 | 43.822 | 1.00 | 20.13 | B |
| ATOM | 3181 | C | LEU | 90 | 24.891 | 3.282 | 45.334 | 1.00 | 24.49 | B |
| ATOM | 3182 | O | LEU | 90 | 23.963 | 4.091 | 45.207 | 1.00 | 24.70 | B |
| ATOM | 3183 | N | ASP | 91 | 24.887 | 2.068 | 44.781 | 1.00 | 25.50 | B |
| ATOM | 3184 | CA | ASP | 91 | 23.765 | 1.617 | 43.975 | 1.00 | 26.54 | B |
| ATOM | 3185 | CB | ASP | 91 | 24.042 | 0.258 | 43.331 | 1.00 | 27.25 | B |
| ATOM | 3186 | CG | ASP | 91 | 24.841 | 0.373 | 42.045 | 1.00 | 29.15 | B |
| ATOM | 3187 | OD1 | ASP | 91 | 24.725 | 1.424 | 41.365 | 1.00 | 28.90 | B |
| ATOM | 3188 | OD2 | ASP | 91 | 25.559 | −0.601 | 41.701 | 1.00 | 29.60 | B |
| ATOM | 3189 | C | ASP | 91 | 22.537 | 1.512 | 44.848 | 1.00 | 27.48 | B |
| ATOM | 3190 | O | ASP | 91 | 21.427 | 1.740 | 44.399 | 1.00 | 28.35 | B |
| ATOM | 3191 | N | GLU | 92 | 22.736 | 1.185 | 46.115 | 1.00 | 28.27 | B |
| ATOM | 3192 | CA | GLU | 92 | 21.603 | 1.065 | 47.018 | 1.00 | 28.89 | B |
| ATOM | 3193 | CB | GLU | 92 | 22.008 | 0.214 | 48.219 | 1.00 | 30.33 | B |
| ATOM | 3194 | CG | GLU | 92 | 20.839 | −0.266 | 49.057 | 1.00 | 33.34 | B |
| ATOM | 3195 | CD | GLU | 92 | 21.141 | −1.578 | 49.772 | 1.00 | 35.27 | B |
| ATOM | 3196 | OE1 | GLU | 92 | 20.340 | −2.000 | 50.633 | 1.00 | 36.65 | B |
| ATOM | 3197 | OE2 | GLU | 92 | 22.181 | −2.200 | 49.469 | 1.00 | 35.05 | B |
| ATOM | 3198 | C | GLU | 92 | 21.106 | 2.459 | 47.424 | 1.00 | 28.34 | B |
| ATOM | 3199 | O | GLU | 92 | 19.897 | 2.685 | 47.581 | 1.00 | 27.53 | B |
| ATOM | 3200 | N | VAL | 93 | 22.037 | 3.395 | 47.585 | 1.00 | 27.17 | B |
| ATOM | 3201 | CA | VAL | 93 | 21.663 | 4.757 | 47.938 | 1.00 | 26.25 | B |
| ATOM | 3202 | CB | VAL | 93 | 22.902 | 5.681 | 48.072 | 1.00 | 27.41 | B |
| ATOM | 3203 | CG1 | VAL | 93 | 22.455 | 7.125 | 48.357 | 1.00 | 27.55 | B |
| ATOM | 3204 | CG2 | VAL | 93 | 23.807 | 5.170 | 49.178 | 1.00 | 29.02 | B |
| ATOM | 3205 | C | VAL | 93 | 20.771 | 5.339 | 46.843 | 1.00 | 24.60 | B |
| ATOM | 3206 | O | VAL | 93 | 19.759 | 5.955 | 47.110 | 1.00 | 24.17 | B |
| ATOM | 3207 | N | ILE | 94 | 21.175 | 5.150 | 45.596 | 1.00 | 22.93 | B |
| ATOM | 3208 | CA | ILE | 94 | 20.398 | 5.657 | 44.466 | 1.00 | 23.06 | B |
| ATOM | 3209 | CB | ILE | 94 | 21.193 | 5.441 | 43.130 | 1.00 | 22.09 | B |
| ATOM | 3210 | CG2 | ILE | 94 | 20.367 | 5.867 | 41.905 | 1.00 | 18.23 | B |
| ATOM | 3211 | CG1 | ILE | 94 | 22.498 | 6.262 | 43.205 | 1.00 | 20.00 | B |
| ATOM | 3212 | CD1 | ILE | 94 | 23.382 | 6.115 | 42.021 | 1.00 | 18.08 | B |
| ATOM | 3213 | C | ILE | 94 | 18.984 | 5.036 | 44.384 | 1.00 | 23.71 | B |
| ATOM | 3214 | O | ILE | 94 | 18.079 | 5.630 | 43.845 | 1.00 | 24.46 | B |
| ATOM | 3215 | N | MET | 95 | 18.787 | 3.839 | 44.924 | 1.00 | 25.14 | B |
| ATOM | 3216 | CA | MET | 95 | 17.451 | 3.234 | 44.893 | 1.00 | 25.03 | B |
| ATOM | 3217 | CB | MET | 95 | 17.511 | 1.735 | 45.167 | 1.00 | 24.81 | B |
| ATOM | 3218 | CG | MET | 95 | 17.896 | 0.898 | 43.984 | 1.00 | 24.81 | B |
| ATOM | 3219 | SD | MET | 95 | 17.840 | −0.821 | 44.434 | 1.00 | 28.44 | B |
| ATOM | 3220 | CE | MET | 95 | 19.568 | −1.182 | 44.778 | 1.00 | 27.32 | B |
| ATOM | 3221 | C | MET | 95 | 16.585 | 3.864 | 45.977 | 1.00 | 25.84 | B |
| ATOM | 3222 | O | MET | 95 | 15.407 | 3.606 | 46.068 | 1.00 | 26.55 | B |
| ATOM | 3223 | N | GLY | 96 | 17.193 | 4.694 | 46.811 | 1.00 | 26.29 | B |
| ATOM | 3224 | CA | GLY | 96 | 16.417 | 5.335 | 47.854 | 1.00 | 26.67 | B |
| ATOM | 3225 | C | GLY | 96 | 16.650 | 4.824 | 49.264 | 1.00 | 28.04 | B |
| ATOM | 3226 | O | GLY | 96 | 15.864 | 5.121 | 50.170 | 1.00 | 29.08 | B |
| ATOM | 3227 | N | TYR | 97 | 17.733 | 4.075 | 49.454 | 1.00 | 28.81 | B |
| ATOM | 3228 | CA | TYR | 97 | 18.081 | 3.524 | 50.760 | 1.00 | 29.52 | B |
| ATOM | 3229 | CB | TYR | 97 | 18.680 | 2.117 | 50.591 | 1.00 | 31.73 | B |
| ATOM | 3230 | CG | TYR | 97 | 17.674 | 1.041 | 50.230 | 1.00 | 34.37 | B |
| ATOM | 3231 | CD1 | TYR | 97 | 17.016 | 0.310 | 51.223 | 1.00 | 35.37 | B |
| ATOM | 3232 | CE1 | TYR | 97 | 16.087 | −0.663 | 50.904 | 1.00 | 36.70 | B |
| ATOM | 3233 | CD2 | TYR | 97 | 17.370 | 0.769 | 48.901 | 1.00 | 35.61 | B |
| ATOM | 3234 | CE2 | TYR | 97 | 16.439 | −0.198 | 48.569 | 1.00 | 37.43 | B |
| ATOM | 3235 | CZ | TYR | 97 | 15.800 | −0.909 | 49.575 | 1.00 | 38.91 | B |
| ATOM | 3236 | OH | TYR | 97 | 14.858 | −1.862 | 49.257 | 1.00 | 40.43 | B |
| ATOM | 3237 | C | TYR | 97 | 19.090 | 4.391 | 51.528 | 1.00 | 28.25 | B |
| ATOM | 3238 | O | TYR | 97 | 19.819 | 5.172 | 50.943 | 1.00 | 29.03 | B |
| ATOM | 3239 | N | ASN | 98 | 19.107 | 4.266 | 52.850 | 1.00 | 26.29 | B |
| ATOM | 3240 | CA | ASN | 98 | 20.087 | 4.993 | 53.646 | 1.00 | 24.16 | B |
| ATOM | 3241 | CB | ASN | 98 | 19.520 | 5.396 | 54.994 | 1.00 | 23.70 | B |
| ATOM | 3242 | CG | ASN | 98 | 18.552 | 6.526 | 54.883 | 1.00 | 21.81 | B |
| ATOM | 3243 | OD1 | ASN | 98 | 18.764 | 7.475 | 54.138 | 1.00 | 20.22 | B |
| ATOM | 3244 | ND2 | ASN | 98 | 17.483 | 6.442 | 55.642 | 1.00 | 22.90 | B |
| ATOM | 3245 | C | ASN | 98 | 21.262 | 4.051 | 53.883 | 1.00 | 22.53 | B |
| ATOM | 3246 | O | ASN | 98 | 21.076 | 2.860 | 54.149 | 1.00 | 23.91 | B |
| ATOM | 3247 | N | CYS | 99 | 22.475 | 4.573 | 53.770 | 1.00 | 20.08 | B |
| ATOM | 3248 | CA | CYS | 99 | 23.652 | 3.741 | 53.976 | 1.00 | 16.35 | B |
| ATOM | 3249 | CB | CYS | 99 | 24.239 | 3.318 | 52.641 | 1.00 | 16.30 | B |
| ATOM | 3250 | SG | CYS | 99 | 23.128 | 2.271 | 51.748 | 1.00 | 16.76 | B |
| ATOM | 3251 | C | CYS | 99 | 24.717 | 4.437 | 54.786 | 1.00 | 13.97 | B |
| ATOM | 3252 | O | CYS | 99 | 24.764 | 5.664 | 54.867 | 1.00 | 13.48 | B |
| ATOM | 3253 | N | THR | 100 | 25.584 | 3.631 | 55.374 | 1.00 | 12.82 | B |
| ATOM | 3254 | CA | THR | 100 | 26.646 | 4.149 | 56.209 | 1.00 | 10.88 | B |
| ATOM | 3255 | CB | THR | 100 | 26.177 | 4.209 | 57.660 | 1.00 | 9.58 | B |
| ATOM | 3256 | OG1 | THR | 100 | 25.155 | 5.204 | 57.768 | 1.00 | 6.29 | B |
| ATOM | 3257 | CG2 | THR | 100 | 27.327 | 4.524 | 58.590 | 1.00 | 10.26 | B |
| ATOM | 3258 | C | THR | 100 | 27.874 | 3.264 | 56.104 | 1.00 | 10.53 | B |
| ATOM | 3259 | O | THR | 100 | 27.764 | 2.056 | 56.040 | 1.00 | 10.24 | B |
| ATOM | 3260 | N | ILE | 101 | 29.044 | 3.890 | 56.059 | 1.00 | 10.89 | B |
| ATOM | 3261 | CA | ILE | 101 | 30.303 | 3.156 | 55.993 | 1.00 | 12.11 | B |
| ATOM | 3262 | CB | ILE | 101 | 31.004 | 3.297 | 54.642 | 1.00 | 13.63 | B |
| ATOM | 3263 | CG2 | ILE | 101 | 32.258 | 2.424 | 54.623 | 1.00 | 13.65 | B |
| ATOM | 3264 | CG1 | ILE | 101 | 30.057 | 2.935 | 53.504 | 1.00 | 15.35 | B |
| ATOM | 3265 | CD1 | ILE | 101 | 30.607 | 3.332 | 52.135 | 1.00 | 15.19 | B |
| ATOM | 3266 | C | ILE | 101 | 31.226 | 3.776 | 57.027 | 1.00 | 11.10 | B |
| ATOM | 3267 | O | ILE | 101 | 31.518 | 4.944 | 56.962 | 1.00 | 13.95 | B |
| ATOM | 3268 | N | PHE | 102 | 31.690 | 2.961 | 57.960 | 1.00 | 8.97 | B |
| ATOM | 3269 | CA | PHE | 102 | 32.569 | 3.412 | 59.024 | 1.00 | 5.36 | B |
| ATOM | 3270 | CB | PHE | 102 | 32.254 | 2.693 | 60.337 | 1.00 | 5.27 | B |
| ATOM | 3271 | CG | PHE | 102 | 30.964 | 3.097 | 60.979 | 1.00 | 3.08 | B |
| ATOM | 3272 | CD1 | PHE | 102 | 30.912 | 4.233 | 61.785 | 1.00 | 3.17 | B |
| ATOM | 3273 | CD2 | PHE | 102 | 29.821 | 2.315 | 60.839 | 1.00 | 1.92 | B |
| ATOM | 3274 | CE1 | PHE | 102 | 29.737 | 4.591 | 62.458 | 1.00 | 2.33 | B |
| ATOM | 3275 | CE2 | PHE | 102 | 28.648 | 2.667 | 61.505 | 1.00 | 1.69 | B |
| ATOM | 3276 | CZ | PHE | 102 | 28.608 | 3.812 | 62.323 | 1.00 | 1.17 | B |
| ATOM | 3277 | C | PHE | 102 | 33.974 | 2.937 | 58.708 | 1.00 | 4.97 | B |
| ATOM | 3278 | O | PHE | 102 | 34.160 | 1.984 | 57.997 | 1.00 | 6.23 | B |
| ATOM | 3279 | N | ALA | 103 | 34.956 | 3.641 | 59.244 | 1.00 | 5.31 | B |
| ATOM | 3280 | CA | ALA | 103 | 36.345 | 3.256 | 59.091 | 1.00 | 3.70 | B |
| ATOM | 3281 | CB | ALA | 103 | 37.115 | 4.337 | 58.408 | 1.00 | 2.97 | B |
| ATOM | 3282 | C | ALA | 103 | 36.781 | 3.126 | 60.546 | 1.00 | 3.79 | B |
| ATOM | 3283 | O | ALA | 103 | 36.811 | 4.105 | 61.266 | 1.00 | 4.80 | B |
| ATOM | 3284 | N | TYR | 104 | 37.086 | 1.908 | 60.981 | 1.00 | 3.80 | B |
| ATOM | 3285 | CA | TYR | 104 | 37.503 | 1.670 | 62.366 | 1.00 | 3.56 | B |
| ATOM | 3286 | CB | TYR | 104 | 36.507 | 0.751 | 63.061 | 1.00 | 2.47 | B |
| ATOM | 3287 | CG | TYR | 104 | 36.842 | 0.498 | 64.507 | 1.00 | 1.59 | B |
| ATOM | 3288 | CD1 | TYR | 104 | 37.780 | −0.465 | 64.875 | 1.00 | 1.99 | B |
| ATOM | 3289 | CE1 | TYR | 104 | 38.079 | −0.706 | 66.227 | 1.00 | 1.00 | B |
| ATOM | 3290 | CD2 | TYR | 104 | 36.211 | 1.215 | 65.510 | 1.00 | 3.23 | B |
| ATOM | 3291 | CE2 | TYR | 104 | 36.492 | 0.988 | 66.863 | 1.00 | 1.00 | B |
| ATOM | 3292 | CZ | TYR | 104 | 37.419 | 0.031 | 67.217 | 1.00 | 1.00 | B |
| ATOM | 3293 | OH | TYR | 104 | 37.667 | −0.164 | 68.555 | 1.00 | 1.00 | B |
| ATOM | 3294 | C | TYR | 104 | 38.893 | 1.046 | 62.517 | 1.00 | 3.38 | B |
| ATOM | 3295 | O | TYR | 104 | 39.225 | 0.087 | 61.843 | 1.00 | 3.35 | B |
| ATOM | 3296 | N | GLY | 105 | 39.680 | 1.586 | 63.440 | 1.00 | 4.31 | B |
| ATOM | 3297 | CA | GLY | 105 | 41.024 | 1.088 | 63.646 | 1.00 | 5.04 | B |
| ATOM | 3298 | C | GLY | 105 | 41.931 | 2.086 | 64.335 | 1.00 | 5.61 | B |
| ATOM | 3299 | O | GLY | 105 | 41.560 | 3.226 | 64.565 | 1.00 | 5.55 | B |
| ATOM | 3300 | N | GLN | 106 | 43.132 | 1.627 | 64.657 | 1.00 | 7.21 | B |
| ATOM | 3301 | CA | GLN | 106 | 44.154 | 2.414 | 65.338 | 1.00 | 9.77 | B |
| ATOM | 3302 | CB | GLN | 106 | 45.303 | 1.473 | 65.701 | 1.00 | 11.84 | B |
| ATOM | 3303 | CG | GLN | 106 | 46.625 | 2.127 | 65.977 | 1.00 | 18.02 | B |
| ATOM | 3304 | CD | GLN | 106 | 47.651 | 1.110 | 66.407 | 1.00 | 20.93 | B |
| ATOM | 3305 | OE1 | GLN | 106 | 47.887 | 0.126 | 65.707 | 1.00 | 20.58 | B |
| ATOM | 3306 | NE2 | GLN | 106 | 48.265 | 1.333 | 67.569 | 1.00 | 24.16 | B |
| ATOM | 3307 | C | GLN | 106 | 44.684 | 3.603 | 64.525 | 1.00 | 9.05 | B |
| ATOM | 3308 | O | GLN | 106 | 44.759 | 3.535 | 63.318 | 1.00 | 8.64 | B |
| ATOM | 3309 | N | THR | 107 | 45.040 | 4.693 | 65.206 | 1.00 | 9.25 | B |
| ATOM | 3310 | CA | THR | 107 | 45.589 | 5.863 | 64.537 | 1.00 | 9.91 | B |
| ATOM | 3311 | CB | THR | 107 | 46.090 | 6.935 | 65.545 | 1.00 | 11.30 | B |
| ATOM | 3312 | OG1 | THR | 107 | 44.998 | 7.433 | 66.328 | 1.00 | 12.57 | B |
| ATOM | 3313 | CG2 | THR | 107 | 46.715 | 8.089 | 64.807 | 1.00 | 11.37 | B |
| ATOM | 3314 | C | THR | 107 | 46.784 | 5.384 | 63.720 | 1.00 | 9.43 | B |
| ATOM | 3315 | O | THR | 107 | 47.631 | 4.615 | 64.226 | 1.00 | 6.62 | B |
| ATOM | 3316 | N | GLY | 108 | 46.836 | 5.797 | 62.455 | 1.00 | 7.40 | B |
| ATOM | 3317 | CA | GLY | 108 | 47.956 | 5.419 | 61.613 | 1.00 | 7.87 | B |
| ATOM | 3318 | C | GLY | 108 | 47.801 | 4.136 | 60.815 | 1.00 | 7.55 | B |
| ATOM | 3319 | O | GLY | 108 | 48.771 | 3.609 | 60.263 | 1.00 | 10.21 | B |
| ATOM | 3320 | N | THR | 109 | 46.581 | 3.624 | 60.748 | 1.00 | 5.82 | B |
| ATOM | 3321 | CA | THR | 109 | 46.349 | 2.400 | 59.992 | 1.00 | 4.83 | B |
| ATOM | 3322 | CB | THR | 109 | 45.588 | 1.329 | 60.827 | 1.00 | 3.30 | B |
| ATOM | 3323 | OG1 | THR | 109 | 44.316 | 1.824 | 61.248 | 1.00 | 2.94 | B |
| ATOM | 3324 | CG2 | THR | 109 | 46.388 | 0.954 | 62.027 | 1.00 | 4.86 | B |
| ATOM | 3325 | C | THR | 109 | 45.611 | 2.616 | 58.675 | 1.00 | 5.10 | B |
| ATOM | 3326 | O | THR | 109 | 45.305 | 1.648 | 57.954 | 1.00 | 5.03 | B |
| ATOM | 3327 | N | GLY | 110 | 45.298 | 3.871 | 58.364 | 1.00 | 3.29 | B |
| ATOM | 3328 | CA | GLY | 110 | 44.613 | 4.141 | 57.122 | 1.00 | 1.90 | B |
| ATOM | 3329 | C | GLY | 110 | 43.131 | 4.484 | 57.097 | 1.00 | 2.61 | B |
| ATOM | 3330 | O | GLY | 110 | 42.521 | 4.385 | 56.025 | 1.00 | 1.00 | B |
| ATOM | 3331 | N | LYS | 111 | 42.539 | 4.885 | 58.227 | 1.00 | 4.13 | B |
| ATOM | 3332 | CA | LYS | 111 | 41.117 | 5.282 | 58.231 | 1.00 | 2.65 | B |
| ATOM | 3333 | CB | LYS | 111 | 40.636 | 5.636 | 59.651 | 1.00 | 2.73 | B |
| ATOM | 3334 | CG | LYS | 111 | 40.588 | 4.463 | 60.630 | 1.00 | 4.22 | B |
| ATOM | 3335 | CD | LYS | 111 | 39.990 | 4.860 | 61.974 | 1.00 | 1.25 | B |
| ATOM | 3336 | CE | LYS | 111 | 40.770 | 5.978 | 62.652 | 1.00 | 1.64 | B |
| ATOM | 3337 | NZ | LYS | 111 | 42.112 | 5.563 | 63.122 | 1.00 | 3.15 | B |
| ATOM | 3338 | C | LYS | 111 | 40.876 | 6.516 | 57.319 | 1.00 | 3.52 | B |
| ATOM | 3339 | O | LYS | 111 | 39.940 | 6.553 | 56.504 | 1.00 | 3.17 | B |
| ATOM | 3340 | N | THR | 112 | 41.738 | 7.515 | 57.421 | 1.00 | 2.71 | B |
| ATOM | 3341 | CA | THR | 112 | 41.536 | 8.697 | 56.607 | 1.00 | 4.38 | B |
| ATOM | 3342 | CB | THR | 112 | 42.245 | 9.927 | 57.209 | 1.00 | 3.24 | B |
| ATOM | 3343 | OG1 | THR | 112 | 41.689 | 10.219 | 58.500 | 1.00 | 2.46 | B |
| ATOM | 3344 | CG2 | THR | 112 | 42.049 | 11.122 | 56.306 | 1.00 | 5.02 | B |
| ATOM | 3345 | C | THR | 112 | 42.010 | 8.459 | 55.175 | 1.00 | 6.62 | B |
| ATOM | 3346 | O | THR | 112 | 41.499 | 9.074 | 54.223 | 1.00 | 5.92 | B |
| ATOM | 3347 | N | PHE | 113 | 42.974 | 7.556 | 55.013 | 1.00 | 7.30 | B |
| ATOM | 3348 | CA | PHE | 113 | 43.484 | 7.275 | 53.680 | 1.00 | 9.51 | B |
| ATOM | 3349 | CB | PHE | 113 | 44.690 | 6.342 | 53.705 | 1.00 | 11.02 | B |
| ATOM | 3350 | CG | PHE | 113 | 45.299 | 6.119 | 52.344 | 1.00 | 13.48 | B |
| ATOM | 3351 | CD1 | PHE | 113 | 46.106 | 7.088 | 51.763 | 1.00 | 13.42 | B |
| ATOM | 3352 | CD2 | PHE | 113 | 45.021 | 4.974 | 51.624 | 1.00 | 13.65 | B |
| ATOM | 3353 | CE1 | PHE | 113 | 46.626 | 6.927 | 50.496 | 1.00 | 13.19 | B |
| ATOM | 3354 | CE2 | PHE | 113 | 45.542 | 4.806 | 50.345 | 1.00 | 14.93 | B |
| ATOM | 3355 | CZ | PHE | 113 | 46.346 | 5.792 | 49.784 | 1.00 | 13.30 | B |
| ATOM | 3356 | C | PHE | 113 | 42.393 | 6.604 | 52.866 | 1.00 | 10.02 | B |
| ATOM | 3357 | O | PHE | 113 | 42.195 | 6.916 | 51.689 | 1.00 | 9.19 | B |
| ATOM | 3358 | N | THR | 114 | 41.686 | 5.686 | 53.519 | 1.00 | 9.92 | B |
| ATOM | 3359 | CA | THR | 114 | 40.601 | 4.946 | 52.905 | 1.00 | 8.86 | B |
| ATOM | 3360 | CB | THR | 114 | 40.157 | 3.792 | 53.812 | 1.00 | 9.97 | B |
| ATOM | 3361 | OG1 | THR | 114 | 41.256 | 2.900 | 54.000 | 1.00 | 10.04 | B |
| ATOM | 3362 | CG2 | THR | 114 | 39.026 | 3.006 | 53.174 | 1.00 | 10.07 | B |
| ATOM | 3363 | C | THR | 114 | 39.397 | 5.824 | 52.608 | 1.00 | 8.06 | B |
| ATOM | 3364 | O | THR | 114 | 38.935 | 5.875 | 51.496 | 1.00 | 8.14 | B |
| ATOM | 3365 | N | MET | 115 | 38.908 | 6.538 | 53.612 | 1.00 | 6.57 | B |
| ATOM | 3366 | CA | MET | 115 | 37.730 | 7.365 | 53.422 | 1.00 | 6.18 | B |
| ATOM | 3367 | CB | MET | 115 | 37.149 | 7.844 | 54.760 | 1.00 | 8.16 | B |
| ATOM | 3368 | CG | MET | 115 | 36.761 | 6.723 | 55.717 | 1.00 | 12.31 | B |
| ATOM | 3369 | SD | MET | 115 | 35.709 | 5.494 | 54.920 | 1.00 | 17.76 | B |
| ATOM | 3370 | CE | MET | 115 | 34.142 | 6.334 | 54.973 | 1.00 | 16.39 | B |
| ATOM | 3371 | C | MET | 115 | 37.903 | 8.594 | 52.570 | 1.00 | 6.31 | B |
| ATOM | 3372 | O | MET | 115 | 36.998 | 8.943 | 51.837 | 1.00 | 10.20 | B |
| ATOM | 3373 | N | GLU | 116 | 39.061 | 9.244 | 52.660 | 1.00 | 6.06 | B |
| ATOM | 3374 | CA | GLU | 116 | 39.295 | 10.476 | 51.909 | 1.00 | 2.45 | B |
| ATOM | 3375 | CB | GLU | 116 | 39.743 | 11.607 | 52.838 | 1.00 | 2.23 | B |
| ATOM | 3376 | CG | GLU | 116 | 38.737 | 11.962 | 53.924 | 1.00 | 1.00 | B |
| ATOM | 3377 | CD | GLU | 116 | 39.091 | 13.216 | 54.722 | 1.00 | 1.00 | B |
| ATOM | 3378 | OE1 | GLU | 116 | 40.124 | 13.850 | 54.464 | 1.00 | 1.56 | B |
| ATOM | 3379 | OE2 | GLU | 116 | 38.323 | 13.586 | 55.626 | 1.00 | 1.00 | B |
| ATOM | 3380 | C | GLU | 116 | 40.342 | 10.311 | 50.843 | 1.00 | 2.04 | B |
| ATOM | 3381 | O | GLU | 116 | 40.070 | 10.587 | 49.695 | 1.00 | 1.54 | B |
| ATOM | 3382 | N | GLY | 117 | 41.539 | 9.869 | 51.235 | 1.00 | 2.71 | B |
| ATOM | 3383 | CA | GLY | 117 | 42.603 | 9.663 | 50.263 | 1.00 | 3.19 | B |
| ATOM | 3384 | C | GLY | 117 | 43.531 | 10.842 | 50.294 | 1.00 | 1.91 | B |
| ATOM | 3385 | O | GLY | 117 | 43.293 | 11.739 | 51.033 | 1.00 | 2.28 | B |
| ATOM | 3386 | N | GLU | 118 | 44.568 | 10.822 | 49.466 | 1.00 | 3.14 | B |
| ATOM | 3387 | CA | GLU | 118 | 45.562 | 11.897 | 49.412 | 1.00 | 3.61 | B |
| ATOM | 3388 | CB | GLU | 118 | 46.879 | 11.427 | 50.051 | 1.00 | 3.14 | B |
| ATOM | 3389 | CG | GLU | 118 | 46.652 | 10.690 | 51.389 | 1.00 | 7.09 | B |
| ATOM | 3390 | CD | GLU | 118 | 47.933 | 10.200 | 52.062 | 1.00 | 9.57 | B |
| ATOM | 3391 | OE1 | GLU | 118 | 48.831 | 9.748 | 51.317 | 1.00 | 11.82 | B |
| ATOM | 3392 | OE2 | GLU | 118 | 48.030 | 10.259 | 53.317 | 1.00 | 6.51 | B |
| ATOM | 3393 | C | GLU | 118 | 45.813 | 12.253 | 47.959 | 1.00 | 4.59 | B |
| ATOM | 3394 | O | GLU | 118 | 45.209 | 11.670 | 47.063 | 1.00 | 4.23 | B |
| ATOM | 3395 | N | ARG | 119 | 46.681 | 13.221 | 47.713 | 1.00 | 7.04 | B |
| ATOM | 3396 | CA | ARG | 119 | 46.976 | 13.564 | 46.329 | 1.00 | 10.62 | B |
| ATOM | 3397 | CB | ARG | 119 | 47.171 | 15.067 | 46.131 | 1.00 | 10.38 | B |
| ATOM | 3398 | CG | ARG | 119 | 45.961 | 15.941 | 46.462 | 1.00 | 13.02 | B |
| ATOM | 3399 | CD | ARG | 119 | 44.705 | 15.414 | 45.837 | 1.00 | 13.25 | B |
| ATOM | 3400 | NE | ARG | 119 | 44.838 | 15.093 | 44.420 | 1.00 | 13.98 | B |
| ATOM | 3401 | CZ | ARG | 119 | 44.759 | 15.955 | 43.411 | 1.00 | 11.43 | B |
| ATOM | 3402 | NH1 | ARG | 119 | 44.543 | 17.247 | 43.614 | 1.00 | 9.13 | B |
| ATOM | 3403 | NH2 | ARG | 119 | 44.890 | 15.498 | 42.175 | 1.00 | 10.86 | B |
| ATOM | 3404 | C | ARG | 119 | 48.274 | 12.907 | 45.912 | 1.00 | 12.67 | B |
| ATOM | 3405 | O | ARG | 119 | 49.210 | 12.823 | 46.712 | 1.00 | 12.43 | B |
| ATOM | 3406 | N | SER | 120 | 48.328 | 12.416 | 44.675 | 1.00 | 15.44 | B |
| ATOM | 3407 | CA | SER | 120 | 49.563 | 11.812 | 44.182 | 1.00 | 17.48 | B |
| ATOM | 3408 | CB | SER | 120 | 49.392 | 11.272 | 42.755 | 1.00 | 18.24 | B |
| ATOM | 3409 | OG | SER | 120 | 48.605 | 10.090 | 42.735 | 1.00 | 19.78 | B |
| ATOM | 3410 | C | SER | 120 | 50.519 | 12.978 | 44.185 | 1.00 | 18.56 | B |
| ATOM | 3411 | O | SER | 120 | 50.161 | 14.050 | 43.772 | 1.00 | 20.75 | B |
| ATOM | 3412 | N | PRO | 121 | 51.748 | 12.782 | 44.660 | 1.00 | 20.06 | B |
| ATOM | 3413 | CD | PRO | 121 | 52.403 | 11.508 | 45.013 | 1.00 | 20.52 | B |
| ATOM | 3414 | CA | PRO | 121 | 52.700 | 13.896 | 44.686 | 1.00 | 20.89 | B |
| ATOM | 3415 | CB | PRO | 121 | 53.912 | 13.275 | 45.385 | 1.00 | 21.27 | B |
| ATOM | 3416 | CG | PRO | 121 | 53.881 | 11.834 | 44.872 | 1.00 | 21.35 | B |
| ATOM | 3417 | C | PRO | 121 | 53.028 | 14.538 | 43.332 | 1.00 | 21.75 | B |
| ATOM | 3418 | O | PRO | 121 | 52.835 | 13.918 | 42.270 | 1.00 | 21.17 | B |
| ATOM | 3419 | N | ASN | 122 | 53.514 | 15.785 | 43.393 | 1.00 | 21.50 | B |
| ATOM | 3420 | CA | ASN | 122 | 53.957 | 16.561 | 42.227 | 1.00 | 22.52 | B |
| ATOM | 3421 | CB | ASN | 122 | 55.199 | 15.865 | 41.632 | 1.00 | 24.29 | B |
| ATOM | 3422 | CG | ASN | 122 | 56.137 | 16.828 | 40.956 | 1.00 | 26.30 | B |
| ATOM | 3423 | OD1 | ASN | 122 | 56.538 | 17.815 | 41.553 | 1.00 | 28.88 | B |
| ATOM | 3424 | ND2 | ASN | 122 | 56.488 | 16.552 | 39.705 | 1.00 | 26.63 | B |
| ATOM | 3425 | C | ASN | 122 | 52.917 | 16.852 | 41.126 | 1.00 | 22.37 | B |
| ATOM | 3426 | O | ASN | 122 | 53.271 | 16.962 | 39.930 | 1.00 | 20.20 | B |
| ATOM | 3427 | N | GLU | 123 | 51.651 | 16.999 | 41.518 | 1.00 | 22.38 | B |
| ATOM | 3428 | CA | GLU | 123 | 50.573 | 17.294 | 40.561 | 1.00 | 22.86 | B |
| ATOM | 3429 | CB | GLU | 123 | 50.664 | 18.735 | 40.072 | 1.00 | 21.58 | B |
| ATOM | 3430 | CG | GLU | 123 | 50.338 | 19.754 | 41.110 | 1.00 | 21.60 | B |
| ATOM | 3431 | CD | GLU | 123 | 50.218 | 21.112 | 40.506 | 1.00 | 23.71 | B |
| ATOM | 3432 | OE1 | GLU | 123 | 51.124 | 21.512 | 39.736 | 1.00 | 24.05 | B |
| ATOM | 3433 | OE2 | GLU | 123 | 49.220 | 21.789 | 40.808 | 1.00 | 24.70 | B |
| ATOM | 3434 | C | GLU | 123 | 50.573 | 16.401 | 39.319 | 1.00 | 23.43 | B |
| ATOM | 3435 | O | GLU | 123 | 50.357 | 16.856 | 38.189 | 1.00 | 22.15 | B |
| ATOM | 3436 | N | GLU | 124 | 50.809 | 15.116 | 39.538 | 1.00 | 25.66 | B |
| ATOM | 3437 | CA | GLU | 124 | 50.840 | 14.186 | 38.435 | 1.00 | 27.17 | B |
| ATOM | 3438 | CB | GLU | 124 | 51.320 | 12.816 | 38.905 | 1.00 | 28.99 | B |
| ATOM | 3439 | CG | GLU | 124 | 51.698 | 11.884 | 37.763 | 1.00 | 33.91 | B |
| ATOM | 3440 | CD | GLU | 124 | 52.179 | 10.531 | 38.247 | 1.00 | 36.81 | B |
| ATOM | 3441 | OE1 | GLU | 124 | 52.681 | 10.475 | 39.395 | 1.00 | 37.60 | B |
| ATOM | 3442 | OE2 | GLU | 124 | 52.061 | 9.543 | 37.476 | 1.00 | 36.71 | B |
| ATOM | 3443 | C | GLU | 124 | 49.466 | 14.045 | 37.791 | 1.00 | 26.54 | B |
| ATOM | 3444 | O | GLU | 124 | 49.351 | 13.966 | 36.571 | 1.00 | 28.04 | B |
| ATOM | 3445 | N | TYR | 125 | 48.425 | 14.023 | 38.616 | 1.00 | 24.51 | B |
| ATOM | 3446 | CA | TYR | 125 | 47.065 | 13.864 | 38.117 | 1.00 | 22.37 | B |
| ATOM | 3447 | CB | TYR | 125 | 46.424 | 12.570 | 38.618 | 1.00 | 24.02 | B |
| ATOM | 3448 | CG | TYR | 125 | 47.232 | 11.305 | 38.445 | 1.00 | 24.34 | B |
| ATOM | 3449 | CD1 | TYR | 125 | 48.215 | 10.951 | 39.372 | 1.00 | 24.16 | B |
| ATOM | 3450 | CE1 | TYR | 125 | 48.938 | 9.770 | 39.238 | 1.00 | 24.97 | B |
| ATOM | 3451 | CD2 | TYR | 125 | 46.994 | 10.440 | 37.368 | 1.00 | 23.29 | B |
| ATOM | 3452 | CE2 | TYR | 125 | 47.715 | 9.257 | 37.224 | 1.00 | 23.28 | B |
| ATOM | 3453 | CZ | TYR | 125 | 48.685 | 8.927 | 38.165 | 1.00 | 25.16 | B |
| ATOM | 3454 | OH | TYR | 125 | 49.395 | 7.750 | 38.059 | 1.00 | 24.88 | B |
| ATOM | 3455 | C | TYR | 125 | 46.089 | 14.936 | 38.586 | 1.00 | 22.58 | B |
| ATOM | 3456 | O | TYR | 125 | 46.366 | 15.703 | 39.516 | 1.00 | 24.23 | B |
| ATOM | 3457 | N | THR | 126 | 44.941 | 14.984 | 37.920 | 1.00 | 21.47 | B |
| ATOM | 3458 | CA | THR | 126 | 43.889 | 15.919 | 38.280 | 1.00 | 20.00 | B |
| ATOM | 3459 | CB | THR | 126 | 42.913 | 16.147 | 37.140 | 1.00 | 20.72 | B |
| ATOM | 3460 | OG1 | THR | 126 | 42.379 | 14.888 | 36.723 | 1.00 | 21.10 | B |
| ATOM | 3461 | CG2 | THR | 126 | 43.598 | 16.837 | 35.984 | 1.00 | 20.85 | B |
| ATOM | 3462 | C | THR | 126 | 43.158 | 15.142 | 39.353 | 1.00 | 17.64 | B |
| ATOM | 3463 | O | THR | 126 | 43.223 | 13.940 | 39.359 | 1.00 | 16.55 | B |
| ATOM | 3464 | N | TRP | 127 | 42.441 | 15.820 | 40.241 | 1.00 | 16.83 | B |
| ATOM | 3465 | CA | TRP | 127 | 41.749 | 15.118 | 41.332 | 1.00 | 15.87 | B |
| ATOM | 3466 | CB | TRP | 127 | 40.927 | 16.080 | 42.213 | 1.00 | 14.78 | B |
| ATOM | 3467 | CG | TRP | 127 | 39.645 | 16.561 | 41.596 | 1.00 | 12.27 | B |
| ATOM | 3468 | CD2 | TRP | 127 | 38.379 | 15.935 | 41.708 | 1.00 | 9.16 | B |
| ATOM | 3469 | CE2 | TRP | 127 | 37.467 | 16.702 | 40.951 | 1.00 | 9.12 | B |
| ATOM | 3470 | CE3 | TRP | 127 | 37.925 | 14.802 | 42.375 | 1.00 | 7.09 | B |
| ATOM | 3471 | CD1 | TRP | 127 | 39.462 | 17.662 | 40.795 | 1.00 | 11.95 | B |
| ATOM | 3472 | NE1 | TRP | 127 | 38.150 | 17.749 | 40.405 | 1.00 | 11.09 | B |
| ATOM | 3473 | CZ2 | TRP | 127 | 36.142 | 16.366 | 40.845 | 1.00 | 8.67 | B |
| ATOM | 3474 | CZ3 | TRP | 127 | 36.606 | 14.472 | 42.271 | 1.00 | 7.96 | B |
| ATOM | 3475 | CH2 | TRP | 127 | 35.724 | 15.251 | 41.511 | 1.00 | 9.12 | B |
| ATOM | 3476 | C | TRP | 127 | 40.824 | 13.969 | 40.917 | 1.00 | 15.77 | B |
| ATOM | 3477 | O | TRP | 127 | 40.807 | 12.907 | 41.536 | 1.00 | 16.78 | B |
| ATOM | 3478 | N | GLU | 128 | 40.065 | 14.145 | 39.855 | 1.00 | 16.83 | B |
| ATOM | 3479 | CA | GLU | 128 | 39.168 | 13.073 | 39.465 | 1.00 | 16.42 | B |
| ATOM | 3480 | CB | GLU | 128 | 38.092 | 13.631 | 38.537 | 1.00 | 15.75 | B |
| ATOM | 3481 | CG | GLU | 128 | 38.578 | 14.230 | 37.234 | 1.00 | 14.47 | B |
| ATOM | 3482 | CD | GLU | 128 | 37.432 | 14.890 | 36.478 | 1.00 | 17.33 | B |
| ATOM | 3483 | OE1 | GLU | 128 | 36.986 | 15.975 | 36.897 | 1.00 | 18.91 | B |
| ATOM | 3484 | OE2 | GLU | 128 | 36.954 | 14.324 | 35.477 | 1.00 | 17.86 | B |
| ATOM | 3485 | C | GLU | 128 | 39.828 | 11.828 | 38.847 | 1.00 | 17.44 | B |
| ATOM | 3486 | O | GLU | 128 | 39.142 | 10.851 | 38.564 | 1.00 | 17.96 | B |
| ATOM | 3487 | N | GLU | 129 | 41.147 | 11.846 | 38.653 | 1.00 | 18.02 | B |
| ATOM | 3488 | CA | GLU | 129 | 41.836 | 10.692 | 38.078 | 1.00 | 19.12 | B |
| ATOM | 3489 | CB | GLU | 129 | 42.509 | 11.020 | 36.740 | 1.00 | 20.74 | B |
| ATOM | 3490 | CG | GLU | 129 | 41.574 | 11.402 | 35.595 | 1.00 | 26.16 | B |
| ATOM | 3491 | CD | GLU | 129 | 42.324 | 11.739 | 34.299 | 1.00 | 30.95 | B |
| ATOM | 3492 | OE1 | GLU | 129 | 41.711 | 12.357 | 33.393 | 1.00 | 32.49 | B |
| ATOM | 3493 | OE2 | GLU | 129 | 43.521 | 11.385 | 34.178 | 1.00 | 32.69 | B |
| ATOM | 3494 | C | GLU | 129 | 42.945 | 10.219 | 38.990 | 1.00 | 18.40 | B |
| ATOM | 3495 | O | GLU | 129 | 43.677 | 9.331 | 38.637 | 1.00 | 18.01 | B |
| ATOM | 3496 | N | ASP | 130 | 43.051 | 10.816 | 40.173 | 1.00 | 17.65 | B |
| ATOM | 3497 | CA | ASP | 130 | 44.115 | 10.465 | 41.113 | 1.00 | 17.80 | B |
| ATOM | 3498 | CB | ASP | 130 | 44.200 | 11.536 | 42.211 | 1.00 | 17.64 | B |
| ATOM | 3499 | CG | ASP | 130 | 45.540 | 11.556 | 42.908 | 1.00 | 19.83 | B |
| ATOM | 3500 | OD1 | ASP | 130 | 46.026 | 10.466 | 43.291 | 1.00 | 20.74 | B |
| ATOM | 3501 | OD2 | ASP | 130 | 46.097 | 12.661 | 43.070 | 1.00 | 20.64 | B |
| ATOM | 3502 | C | ASP | 130 | 43.843 | 9.091 | 41.704 | 1.00 | 17.66 | B |
| ATOM | 3503 | O | ASP | 130 | 42.792 | 8.867 | 42.302 | 1.00 | 18.25 | B |
| ATOM | 3504 | N | PRO | 131 | 44.778 | 8.141 | 41.521 | 1.00 | 17.22 | B |
| ATOM | 3505 | CD | PRO | 131 | 46.046 | 8.282 | 40.780 | 1.00 | 17.06 | B |
| ATOM | 3506 | CA | PRO | 131 | 44.617 | 6.778 | 42.052 | 1.00 | 16.05 | B |
| ATOM | 3507 | CB | PRO | 131 | 45.716 | 5.994 | 41.316 | 1.00 | 14.70 | B |
| ATOM | 3508 | CG | PRO | 131 | 46.802 | 7.019 | 41.154 | 1.00 | 17.48 | B |
| ATOM | 3509 | C | PRO | 131 | 44.668 | 6.713 | 43.589 | 1.00 | 15.30 | B |
| ATOM | 3510 | O | PRO | 131 | 44.318 | 5.697 | 44.187 | 1.00 | 14.37 | B |
| ATOM | 3511 | N | LEU | 132 | 45.114 | 7.797 | 44.226 | 1.00 | 15.18 | B |
| ATOM | 3512 | CA | LEU | 132 | 45.169 | 7.841 | 45.683 | 1.00 | 13.57 | B |
| ATOM | 3513 | CB | LEU | 132 | 46.380 | 8.644 | 46.165 | 1.00 | 12.21 | B |
| ATOM | 3514 | CG | LEU | 132 | 47.741 | 8.012 | 45.842 | 1.00 | 12.83 | B |
| ATOM | 3515 | CD1 | LEU | 132 | 48.850 | 8.803 | 46.511 | 1.00 | 7.88 | B |
| ATOM | 3516 | CD2 | LEU | 132 | 47.773 | 6.553 | 46.317 | 1.00 | 13.99 | B |
| ATOM | 3517 | C | LEU | 132 | 43.882 | 8.393 | 46.295 | 1.00 | 14.28 | B |
| ATOM | 3518 | O | LEU | 132 | 43.737 | 8.410 | 47.526 | 1.00 | 13.98 | B |
| ATOM | 3519 | N | ALA | 133 | 42.947 | 8.832 | 45.443 | 1.00 | 13.83 | B |
| ATOM | 3520 | CA | ALA | 133 | 41.651 | 9.342 | 45.909 | 1.00 | 12.82 | B |
| ATOM | 3521 | CB | ALA | 133 | 40.796 | 9.805 | 44.733 | 1.00 | 12.54 | B |
| ATOM | 3522 | C | ALA | 133 | 40.875 | 8.291 | 46.717 | 1.00 | 13.00 | B |
| ATOM | 3523 | O | ALA | 133 | 40.840 | 7.092 | 46.371 | 1.00 | 14.00 | B |
| ATOM | 3524 | N | GLY | 134 | 40.226 | 8.760 | 47.780 | 1.00 | 13.17 | B |
| ATOM | 3525 | CA | GLY | 134 | 39.470 | 7.884 | 48.653 | 1.00 | 10.45 | B |
| ATOM | 3526 | C | GLY | 134 | 37.996 | 7.819 | 48.324 | 1.00 | 9.48 | B |
| ATOM | 3527 | O | GLY | 134 | 37.546 | 8.422 | 47.385 | 1.00 | 8.50 | B |
| ATOM | 3528 | N | ILE | 135 | 37.254 | 7.094 | 49.158 | 1.00 | 10.67 | B |
| ATOM | 3529 | CA | ILE | 135 | 35.820 | 6.874 | 48.981 | 1.00 | 9.46 | B |
| ATOM | 3530 | CB | ILE | 135 | 35.237 | 6.087 | 50.180 | 1.00 | 9.70 | B |
| ATOM | 3531 | CG2 | ILE | 135 | 33.709 | 5.990 | 50.079 | 1.00 | 10.21 | B |
| ATOM | 3532 | CG1 | ILE | 135 | 35.837 | 4.686 | 50.214 | 1.00 | 8.19 | B |
| ATOM | 3533 | CD1 | ILE | 135 | 35.426 | 3.864 | 51.452 | 1.00 | 8.61 | B |
| ATOM | 3534 | C | ILE | 135 | 34.968 | 8.115 | 48.739 | 1.00 | 9.92 | B |
| ATOM | 3535 | O | ILE | 135 | 34.135 | 8.150 | 47.812 | 1.00 | 7.51 | B |
| ATOM | 3536 | N | ILE | 136 | 35.157 | 9.136 | 49.560 | 1.00 | 9.63 | B |
| ATOM | 3537 | CA | ILE | 136 | 34.379 | 10.340 | 49.371 | 1.00 | 8.14 | B |
| ATOM | 3538 | CB | ILE | 136 | 34.671 | 11.371 | 50.500 | 1.00 | 6.28 | B |
| ATOM | 3539 | CG2 | ILE | 136 | 33.997 | 12.691 | 50.166 | 1.00 | 6.74 | B |
| ATOM | 3540 | CG1 | ILE | 136 | 34.125 | 10.825 | 51.831 | 1.00 | 5.22 | B |
| ATOM | 3541 | CD1 | ILE | 136 | 34.553 | 11.574 | 53.070 | 1.00 | 1.00 | B |
| ATOM | 3542 | C | ILE | 136 | 34.538 | 10.992 | 47.978 | 1.00 | 9.33 | B |
| ATOM | 3543 | O | ILE | 136 | 33.569 | 11.242 | 47.274 | 1.00 | 10.23 | B |
| ATOM | 3544 | N | PRO | 137 | 35.767 | 11.252 | 47.552 | 1.00 | 7.86 | B |
| ATOM | 3545 | CD | PRO | 137 | 37.096 | 11.215 | 48.163 | 1.00 | 7.00 | B |
| ATOM | 3546 | CA | PRO | 137 | 35.816 | 11.874 | 46.234 | 1.00 | 7.00 | B |
| ATOM | 3547 | CB | PRO | 137 | 37.243 | 12.398 | 46.174 | 1.00 | 5.68 | B |
| ATOM | 3548 | CG | PRO | 137 | 37.968 | 11.448 | 46.976 | 1.00 | 7.36 | B |
| ATOM | 3549 | C | PRO | 137 | 35.370 | 10.967 | 45.098 | 1.00 | 7.27 | B |
| ATOM | 3550 | O | PRO | 137 | 34.857 | 11.434 | 44.120 | 1.00 | 9.92 | B |
| ATOM | 3551 | N | ARG | 138 | 35.547 | 9.661 | 45.233 | 1.00 | 7.38 | B |
| ATOM | 3552 | CA | ARG | 138 | 35.132 | 8.765 | 44.157 | 1.00 | 4.69 | B |
| ATOM | 3553 | CB | ARG | 138 | 35.761 | 7.375 | 44.314 | 1.00 | 5.18 | B |
| ATOM | 3554 | CG | ARG | 138 | 37.257 | 7.373 | 44.145 | 1.00 | 4.97 | B |
| ATOM | 3555 | CD | ARG | 138 | 37.858 | 6.057 | 44.522 | 1.00 | 8.61 | B |
| ATOM | 3556 | NE | ARG | 138 | 39.307 | 6.094 | 44.387 | 1.00 | 9.73 | B |
| ATOM | 3557 | CZ | ARG | 138 | 39.954 | 5.973 | 43.235 | 1.00 | 12.02 | B |
| ATOM | 3558 | NH1 | ARG | 138 | 39.279 | 5.799 | 42.102 | 1.00 | 12.04 | B |
| ATOM | 3559 | NH2 | ARG | 138 | 41.280 | 6.028 | 43.216 | 1.00 | 13.69 | B |
| ATOM | 3560 | C | ARG | 138 | 33.623 | 8.667 | 44.131 | 1.00 | 4.18 | B |
| ATOM | 3561 | O | ARG | 138 | 33.017 | 8.611 | 43.094 | 1.00 | 7.46 | B |
| ATOM | 3562 | N | THR | 139 | 33.013 | 8.666 | 45.295 | 1.00 | 3.72 | B |
| ATOM | 3563 | CA | THR | 139 | 31.578 | 8.581 | 45.339 | 1.00 | 3.48 | B |
| ATOM | 3564 | CB | THR | 139 | 31.103 | 8.436 | 46.792 | 1.00 | 2.17 | B |
| ATOM | 3565 | OG1 | THR | 139 | 31.647 | 7.220 | 47.321 | 1.00 | 4.08 | B |
| ATOM | 3566 | CG2 | THR | 139 | 29.586 | 8.366 | 46.872 | 1.00 | 1.00 | B |
| ATOM | 3567 | C | THR | 139 | 30.956 | 9.798 | 44.677 | 1.00 | 4.20 | B |
| ATOM | 3568 | O | THR | 139 | 30.178 | 9.666 | 43.727 | 1.00 | 5.38 | B |
| ATOM | 3569 | N | LEU | 140 | 31.313 | 10.983 | 45.148 | 1.00 | 4.85 | B |
| ATOM | 3570 | CA | LEU | 140 | 30.740 | 12.187 | 44.582 | 1.00 | 5.86 | B |
| ATOM | 3571 | CB | LEU | 140 | 31.374 | 13.423 | 45.207 | 1.00 | 4.02 | B |
| ATOM | 3572 | CG | LEU | 140 | 30.995 | 13.484 | 46.692 | 1.00 | 4.42 | B |
| ATOM | 3573 | CD1 | LEU | 140 | 31.695 | 14.631 | 47.363 | 1.00 | 6.86 | B |
| ATOM | 3574 | CD2 | LEU | 140 | 29.511 | 13.617 | 46.827 | 1.00 | 2.19 | B |
| ATOM | 3575 | C | LEU | 140 | 30.902 | 12.211 | 43.091 | 1.00 | 8.32 | B |
| ATOM | 3576 | O | LEU | 140 | 29.958 | 12.523 | 42.378 | 1.00 | 10.70 | B |
| ATOM | 3577 | N | HIS | 141 | 32.085 | 11.853 | 42.611 | 1.00 | 9.41 | B |
| ATOM | 3578 | CA | HIS | 141 | 32.315 | 11.876 | 41.180 | 1.00 | 11.42 | B |
| ATOM | 3579 | CB | HIS | 141 | 33.753 | 11.465 | 40.836 | 1.00 | 12.95 | B |
| ATOM | 3580 | CG | HIS | 141 | 34.064 | 11.523 | 39.364 | 1.00 | 15.31 | B |
| ATOM | 3581 | CD2 | HIS | 141 | 34.074 | 10.555 | 38.413 | 1.00 | 14.59 | B |
| ATOM | 3582 | ND1 | HIS | 141 | 34.404 | 12.693 | 38.713 | 1.00 | 17.05 | B |
| ATOM | 3583 | CE1 | HIS | 141 | 34.612 | 12.445 | 37.432 | 1.00 | 15.66 | B |
| ATOM | 3584 | NE2 | HIS | 141 | 34.418 | 11.154 | 37.225 | 1.00 | 15.55 | B |
| ATOM | 3585 | C | HIS | 141 | 31.362 | 10.910 | 40.495 | 1.00 | 11.46 | B |
| ATOM | 3586 | O | HIS | 141 | 30.727 | 11.239 | 39.499 | 1.00 | 12.67 | B |
| ATOM | 3587 | N | GLN | 142 | 31.251 | 9.714 | 41.054 | 1.00 | 12.56 | B |
| ATOM | 3588 | CA | GLN | 142 | 30.405 | 8.694 | 40.464 | 1.00 | 12.86 | B |
| ATOM | 3589 | CB | GLN | 142 | 30.707 | 7.336 | 41.103 | 1.00 | 14.29 | B |
| ATOM | 3590 | CG | GLN | 142 | 32.000 | 6.739 | 40.590 | 1.00 | 18.45 | B |
| ATOM | 3591 | CD | GLN | 142 | 32.012 | 6.628 | 39.068 | 1.00 | 21.75 | B |
| ATOM | 3592 | OE1 | GLN | 142 | 31.349 | 5.751 | 38.489 | 1.00 | 23.11 | B |
| ATOM | 3593 | NE2 | GLN | 142 | 32.743 | 7.535 | 38.408 | 1.00 | 20.86 | B |
| ATOM | 3594 | C | GLN | 142 | 28.915 | 8.984 | 40.473 | 1.00 | 12.11 | B |
| ATOM | 3595 | O | GLN | 142 | 28.206 | 8.585 | 39.560 | 1.00 | 11.87 | B |
| ATOM | 3596 | N | ILE | 143 | 28.434 | 9.664 | 41.506 | 1.00 | 11.12 | B |
| ATOM | 3597 | CA | ILE | 143 | 27.018 | 10.010 | 41.573 | 1.00 | 12.39 | B |
| ATOM | 3598 | CB | ILE | 143 | 26.722 | 10.953 | 42.788 | 1.00 | 12.55 | B |
| ATOM | 3599 | CG2 | ILE | 143 | 25.341 | 11.608 | 42.650 | 1.00 | 12.75 | B |
| ATOM | 3600 | CG1 | ILE | 143 | 26.784 | 10.147 | 44.093 | 1.00 | 13.10 | B |
| ATOM | 3601 | CD1 | ILE | 143 | 26.532 | 10.971 | 45.338 | 1.00 | 10.72 | B |
| ATOM | 3602 | C | ILE | 143 | 26.587 | 10.710 | 40.275 | 1.00 | 13.82 | B |
| ATOM | 3603 | O | ILE | 143 | 25.541 | 10.391 | 39.705 | 1.00 | 14.18 | B |
| ATOM | 3604 | N | PHE | 144 | 27.397 | 11.666 | 39.816 | 1.00 | 14.48 | B |
| ATOM | 3605 | CA | PHE | 144 | 27.099 | 12.430 | 38.605 | 1.00 | 15.02 | B |
| ATOM | 3606 | CB | PHE | 144 | 28.023 | 13.646 | 38.513 | 1.00 | 14.03 | B |
| ATOM | 3607 | CG | PHE | 144 | 27.773 | 14.676 | 39.585 | 1.00 | 12.67 | B |
| ATOM | 3608 | CD1 | PHE | 144 | 26.680 | 15.527 | 39.510 | 1.00 | 10.36 | B |
| ATOM | 3609 | CD2 | PHE | 144 | 28.623 | 14.796 | 40.678 | 1.00 | 13.84 | B |
| ATOM | 3610 | CE1 | PHE | 144 | 26.442 | 16.473 | 40.498 | 1.00 | 9.69 | B |
| ATOM | 3611 | CE2 | PHE | 144 | 28.375 | 15.761 | 41.680 | 1.00 | 13.70 | B |
| ATOM | 3612 | CZ | PHE | 144 | 27.286 | 16.591 | 41.578 | 1.00 | 11.21 | B |
| ATOM | 3613 | C | PHE | 144 | 27.223 | 11.586 | 37.348 | 1.00 | 16.57 | B |
| ATOM | 3614 | O | PHE | 144 | 26.516 | 11.835 | 36.384 | 1.00 | 16.66 | B |
| ATOM | 3615 | N | GLU | 145 | 28.123 | 10.593 | 37.364 | 1.00 | 20.10 | B |
| ATOM | 3616 | CA | GLU | 145 | 28.335 | 9.691 | 36.210 | 1.00 | 22.03 | B |
| ATOM | 3617 | CB | GLU | 145 | 29.597 | 8.825 | 36.352 | 1.00 | 26.12 | B |
| ATOM | 3618 | CG | GLU | 145 | 30.902 | 9.538 | 36.044 | 1.00 | 32.68 | B |
| ATOM | 3619 | CD | GLU | 145 | 31.004 | 9.949 | 34.595 | 1.00 | 36.87 | B |
| ATOM | 3620 | OE1 | GLU | 145 | 31.965 | 10.666 | 34.249 | 1.00 | 39.57 | B |
| ATOM | 3621 | OE2 | GLU | 145 | 30.121 | 9.549 | 33.807 | 1.00 | 40.00 | B |
| ATOM | 3622 | C | GLU | 145 | 27.194 | 8.705 | 36.029 | 1.00 | 21.04 | B |
| ATOM | 3623 | O | GLU | 145 | 26.750 | 8.470 | 34.943 | 1.00 | 20.94 | B |
| ATOM | 3624 | N | LYS | 146 | 26.728 | 8.129 | 37.127 | 1.00 | 22.01 | B |
| ATOM | 3625 | CA | LYS | 146 | 25.628 | 7.166 | 37.072 | 1.00 | 22.94 | B |
| ATOM | 3626 | CB | LYS | 146 | 25.489 | 6.433 | 38.423 | 1.00 | 24.69 | B |
| ATOM | 3627 | CG | LYS | 146 | 26.725 | 5.599 | 38.799 | 1.00 | 27.30 | B |
| ATOM | 3628 | CD | LYS | 146 | 26.480 | 4.519 | 39.854 | 1.00 | 24.53 | B |
| ATOM | 3629 | CE | LYS | 146 | 27.560 | 3.447 | 39.715 | 1.00 | 25.61 | B |
| ATOM | 3630 | NZ | LYS | 146 | 27.404 | 2.262 | 40.595 | 1.00 | 24.71 | B |
| ATOM | 3631 | C | LYS | 146 | 24.281 | 7.799 | 36.702 | 1.00 | 24.00 | B |
| ATOM | 3632 | O | LYS | 146 | 23.472 | 7.178 | 36.020 | 1.00 | 24.07 | B |
| ATOM | 3633 | N | LEU | 147 | 24.049 | 9.035 | 37.138 | 1.00 | 23.75 | B |
| ATOM | 3634 | CA | LEU | 147 | 22.788 | 9.720 | 36.850 | 1.00 | 24.08 | B |
| ATOM | 3635 | CB | LEU | 147 | 22.247 | 10.365 | 38.123 | 1.00 | 24.33 | B |
| ATOM | 3636 | CG | LEU | 147 | 21.976 | 9.460 | 39.325 | 1.00 | 24.88 | B |
| ATOM | 3637 | CD1 | LEU | 147 | 21.607 | 10.299 | 40.537 | 1.00 | 24.59 | B |
| ATOM | 3638 | CD2 | LEU | 147 | 20.847 | 8.493 | 39.014 | 1.00 | 24.04 | B |
| ATOM | 3639 | C | LEU | 147 | 22.895 | 10.796 | 35.762 | 1.00 | 25.02 | B |
| ATOM | 3640 | O | LEU | 147 | 22.110 | 11.755 | 35.736 | 1.00 | 22.56 | B |
| ATOM | 3641 | N | THR | 148 | 23.857 | 10.627 | 34.857 | 1.00 | 27.04 | B |
| ATOM | 3642 | CA | THR | 148 | 24.073 | 11.585 | 33.774 | 1.00 | 28.40 | B |
| ATOM | 3643 | CB | THR | 148 | 25.296 | 11.194 | 32.905 | 1.00 | 28.80 | B |
| ATOM | 3644 | OG1 | THR | 148 | 25.479 | 12.150 | 31.850 | 1.00 | 29.27 | B |
| ATOM | 3645 | CG2 | THR | 148 | 25.108 | 9.794 | 32.318 | 1.00 | 30.26 | B |
| ATOM | 3646 | C | THR | 148 | 22.855 | 11.738 | 32.865 | 1.00 | 28.70 | B |
| ATOM | 3647 | O | THR | 148 | 22.466 | 12.848 | 32.580 | 1.00 | 29.54 | B |
| ATOM | 3648 | N | ASP | 149 | 22.253 | 10.638 | 32.413 | 1.00 | 27.95 | B |
| ATOM | 3649 | CA | ASP | 149 | 21.087 | 10.749 | 31.533 | 1.00 | 28.50 | B |
| ATOM | 3650 | CB | ASP | 149 | 21.500 | 11.014 | 30.067 | 1.00 | 28.76 | B |
| ATOM | 3651 | CG | ASP | 149 | 22.520 | 10.010 | 29.522 | 1.00 | 29.99 | B |
| ATOM | 3652 | OD1 | ASP | 149 | 22.501 | 8.830 | 29.939 | 1.00 | 29.75 | B |
| ATOM | 3653 | OD2 | ASP | 149 | 23.332 | 10.408 | 28.646 | 1.00 | 29.41 | B |
| ATOM | 3654 | C | ASP | 149 | 20.148 | 9.551 | 31.576 | 1.00 | 28.84 | B |
| ATOM | 3655 | O | ASP | 149 | 19.636 | 9.096 | 30.555 | 1.00 | 27.84 | B |
| ATOM | 3656 | N | ASN | 150 | 19.899 | 9.055 | 32.778 | 1.00 | 29.57 | B |
| ATOM | 3657 | CA | ASN | 150 | 19.008 | 7.912 | 32.928 | 1.00 | 31.21 | B |
| ATOM | 3658 | CB | ASN | 150 | 19.483 | 7.010 | 34.080 | 1.00 | 29.55 | B |
| ATOM | 3659 | CG | ASN | 150 | 19.259 | 7.641 | 35.459 | 1.00 | 28.21 | B |
| ATOM | 3660 | OD1 | ASN | 150 | 19.347 | 8.859 | 35.618 | 1.00 | 27.26 | B |
| ATOM | 3661 | ND2 | ASN | 150 | 18.969 | 6.804 | 36.458 | 1.00 | 25.05 | B |
| ATOM | 3662 | C | ASN | 150 | 17.550 | 8.345 | 33.175 | 1.00 | 31.80 | B |
| ATOM | 3663 | O | ASN | 150 | 16.693 | 7.501 | 33.485 | 1.00 | 32.95 | B |
| ATOM | 3664 | N | GLY | 151 | 17.279 | 9.648 | 33.043 | 1.00 | 30.56 | B |
| ATOM | 3665 | CA | GLY | 151 | 15.939 | 10.169 | 33.247 | 1.00 | 29.70 | B |
| ATOM | 3666 | C | GLY | 151 | 15.601 | 10.387 | 34.701 | 1.00 | 29.38 | B |
| ATOM | 3667 | O | GLY | 151 | 14.462 | 10.518 | 35.052 | 1.00 | 29.95 | B |
| ATOM | 3668 | N | THR | 152 | 16.616 | 10.412 | 35.549 | 1.00 | 29.90 | B |
| ATOM | 3669 | CA | THR | 152 | 16.386 | 10.634 | 36.964 | 1.00 | 30.17 | B |
| ATOM | 3670 | CB | THR | 152 | 17.082 | 9.552 | 37.805 | 1.00 | 29.93 | B |
| ATOM | 3671 | OG1 | THR | 152 | 16.662 | 8.249 | 37.373 | 1.00 | 29.92 | B |
| ATOM | 3672 | CG2 | THR | 152 | 16.739 | 9.730 | 39.272 | 1.00 | 31.14 | B |
| ATOM | 3673 | C | THR | 152 | 16.902 | 12.022 | 37.384 | 1.00 | 31.11 | B |
| ATOM | 3674 | O | THR | 152 | 18.104 | 12.232 | 37.543 | 1.00 | 32.13 | B |
| ATOM | 3675 | N | GLU | 153 | 15.977 | 12.968 | 37.531 | 1.00 | 30.29 | B |
| ATOM | 3676 | CA | GLU | 153 | 16.310 | 14.325 | 37.948 | 1.00 | 28.58 | B |
| ATOM | 3677 | CB | GLU | 153 | 15.041 | 15.174 | 37.977 | 1.00 | 31.74 | B |
| ATOM | 3678 | CG | GLU | 153 | 15.257 | 16.669 | 37.853 | 1.00 | 35.57 | B |
| ATOM | 3679 | CD | GLU | 153 | 15.641 | 17.082 | 36.438 | 1.00 | 38.01 | B |
| ATOM | 3680 | OE1 | GLU | 153 | 15.923 | 18.281 | 36.200 | 1.00 | 38.59 | B |
| ATOM | 3681 | OE2 | GLU | 153 | 15.655 | 16.201 | 35.551 | 1.00 | 39.17 | B |
| ATOM | 3682 | C | GLU | 153 | 16.861 | 14.173 | 39.366 | 1.00 | 25.90 | B |
| ATOM | 3683 | O | GLU | 153 | 16.382 | 13.346 | 40.114 | 1.00 | 25.18 | B |
| ATOM | 3684 | N | PHE | 154 | 17.852 | 14.978 | 39.738 | 1.00 | 24.45 | B |
| ATOM | 3685 | CA | PHE | 154 | 18.447 | 14.852 | 41.074 | 1.00 | 21.39 | B |
| ATOM | 3686 | CB | PHE | 154 | 19.411 | 13.651 | 41.115 | 1.00 | 20.65 | B |
| ATOM | 3687 | CG | PHE | 154 | 20.679 | 13.846 | 40.306 | 1.00 | 20.31 | B |
| ATOM | 3688 | CD1 | PHE | 154 | 21.853 | 14.284 | 40.904 | 1.00 | 19.86 | B |
| ATOM | 3689 | CD2 | PHE | 154 | 20.698 | 13.570 | 38.945 | 1.00 | 19.64 | B |
| ATOM | 3690 | CE1 | PHE | 154 | 23.021 | 14.435 | 40.142 | 1.00 | 21.56 | B |
| ATOM | 3691 | CE2 | PHE | 154 | 21.856 | 13.720 | 38.194 | 1.00 | 20.70 | B |
| ATOM | 3692 | CZ | PHE | 154 | 23.017 | 14.149 | 38.786 | 1.00 | 19.85 | B |
| ATOM | 3693 | C | PHE | 154 | 19.224 | 16.073 | 41.567 | 1.00 | 19.03 | B |
| ATOM | 3694 | O | PHE | 154 | 19.579 | 16.970 | 40.805 | 1.00 | 18.07 | B |
| ATOM | 3695 | N | SER | 155 | 19.470 | 16.107 | 42.865 | 1.00 | 17.25 | B |
| ATOM | 3696 | CA | SER | 155 | 20.234 | 17.200 | 43.451 | 1.00 | 17.56 | B |
| ATOM | 3697 | CB | SER | 155 | 19.310 | 18.302 | 44.043 | 1.00 | 18.40 | B |
| ATOM | 3698 | OG | SER | 155 | 18.744 | 17.999 | 45.315 | 1.00 | 19.07 | B |
| ATOM | 3699 | C | SER | 155 | 21.072 | 16.536 | 44.521 | 1.00 | 16.97 | B |
| ATOM | 3700 | O | SER | 155 | 20.629 | 15.587 | 45.157 | 1.00 | 15.32 | B |
| ATOM | 3701 | N | VAL | 156 | 22.286 | 17.034 | 44.708 | 1.00 | 17.21 | B |
| ATOM | 3702 | CA | VAL | 156 | 23.181 | 16.479 | 45.709 | 1.00 | 15.73 | B |
| ATOM | 3703 | CB | VAL | 156 | 24.452 | 15.964 | 45.066 | 1.00 | 16.35 | B |
| ATOM | 3704 | CG1 | VAL | 156 | 25.307 | 15.319 | 46.089 | 1.00 | 16.70 | B |
| ATOM | 3705 | CG2 | VAL | 156 | 24.117 | 14.993 | 43.973 | 1.00 | 18.36 | B |
| ATOM | 3706 | C | VAL | 156 | 23.577 | 17.503 | 46.762 | 1.00 | 14.63 | B |
| ATOM | 3707 | O | VAL | 156 | 24.031 | 18.595 | 46.441 | 1.00 | 12.84 | B |
| ATOM | 3708 | N | LYS | 157 | 23.394 | 17.138 | 48.024 | 1.00 | 15.08 | B |
| ATOM | 3709 | CA | LYS | 157 | 23.739 | 18.019 | 49.139 | 1.00 | 16.33 | B |
| ATOM | 3710 | CB | LYS | 157 | 22.485 | 18.370 | 49.962 | 1.00 | 17.27 | B |
| ATOM | 3711 | CG | LYS | 157 | 21.640 | 19.492 | 49.381 | 1.00 | 19.38 | B |
| ATOM | 3712 | CD | LYS | 157 | 20.323 | 19.704 | 50.121 | 1.00 | 19.23 | B |
| ATOM | 3713 | CE | LYS | 157 | 19.563 | 20.911 | 49.535 | 1.00 | 20.48 | B |
| ATOM | 3714 | NZ | LYS | 157 | 20.216 | 22.239 | 49.815 | 1.00 | 19.89 | B |
| ATOM | 3715 | C | LYS | 157 | 24.738 | 17.288 | 50.025 | 1.00 | 15.63 | B |
| ATOM | 3716 | O | LYS | 157 | 24.568 | 16.118 | 50.305 | 1.00 | 17.71 | B |
| ATOM | 3717 | N | VAL | 158 | 25.789 | 17.979 | 50.447 | 1.00 | 14.09 | B |
| ATOM | 3718 | CA | VAL | 158 | 26.782 | 17.350 | 51.313 | 1.00 | 12.31 | B |
| ATOM | 3719 | CB | VAL | 158 | 28.184 | 17.314 | 50.670 | 1.00 | 11.69 | B |
| ATOM | 3720 | CG1 | VAL | 158 | 28.150 | 16.490 | 49.405 | 1.00 | 12.25 | B |
| ATOM | 3721 | CG2 | VAL | 158 | 28.657 | 18.731 | 50.367 | 1.00 | 11.55 | B |
| ATOM | 3722 | C | VAL | 158 | 26.911 | 18.070 | 52.636 | 1.00 | 11.94 | B |
| ATOM | 3723 | O | VAL | 158 | 26.668 | 19.270 | 52.726 | 1.00 | 11.97 | B |
| ATOM | 3724 | N | SER | 159 | 27.301 | 17.321 | 53.659 | 1.00 | 10.91 | B |
| ATOM | 3725 | CA | SER | 159 | 27.490 | 17.876 | 54.992 | 1.00 | 11.22 | B |
| ATOM | 3726 | CB | SER | 159 | 26.245 | 17.662 | 55.846 | 1.00 | 11.02 | B |
| ATOM | 3727 | OG | SER | 159 | 25.184 | 18.476 | 55.385 | 1.00 | 17.68 | B |
| ATOM | 3728 | C | SER | 159 | 28.677 | 17.212 | 55.667 | 1.00 | 11.18 | B |
| ATOM | 3729 | O | SER | 159 | 28.925 | 16.002 | 55.499 | 1.00 | 10.26 | B |
| ATOM | 3730 | N | LEU | 160 | 29.431 | 18.011 | 56.405 | 1.00 | 11.19 | B |
| ATOM | 3731 | CA | LEU | 160 | 30.583 | 17.495 | 57.115 | 1.00 | 11.64 | B |
| ATOM | 3732 | CB | LEU | 160 | 31.875 | 18.043 | 56.498 | 1.00 | 11.99 | B |
| ATOM | 3733 | CG | LEU | 160 | 33.168 | 17.440 | 57.061 | 1.00 | 12.29 | B |
| ATOM | 3734 | CD1 | LEU | 160 | 33.088 | 15.915 | 57.170 | 1.00 | 12.16 | B |
| ATOM | 3735 | CD2 | LEU | 160 | 34.307 | 17.848 | 56.170 | 1.00 | 13.02 | B |
| ATOM | 3736 | C | LEU | 160 | 30.476 | 17.836 | 58.606 | 1.00 | 12.31 | B |
| ATOM | 3737 | O | LEU | 160 | 30.894 | 18.913 | 59.056 | 1.00 | 13.72 | B |
| ATOM | 3738 | N | LEU | 161 | 29.921 | 16.899 | 59.365 | 1.00 | 11.68 | B |
| ATOM | 3739 | CA | LEU | 161 | 29.728 | 17.056 | 60.794 | 1.00 | 11.73 | B |
| ATOM | 3740 | CB | LEU | 161 | 28.387 | 16.462 | 61.184 | 1.00 | 10.86 | B |
| ATOM | 3741 | CG | LEU | 161 | 28.069 | 16.373 | 62.667 | 1.00 | 11.21 | B |
| ATOM | 3742 | CD1 | LEU | 161 | 28.038 | 17.772 | 63.257 | 1.00 | 14.64 | B |
| ATOM | 3743 | CD2 | LEU | 161 | 26.735 | 15.687 | 62.849 | 1.00 | 11.87 | B |
| ATOM | 3744 | C | LEU | 161 | 30.805 | 16.318 | 61.565 | 1.00 | 11.76 | B |
| ATOM | 3745 | O | LEU | 161 | 31.023 | 15.148 | 61.353 | 1.00 | 14.92 | B |
| ATOM | 3746 | N | GLU | 162 | 31.493 | 17.005 | 62.461 | 1.00 | 11.26 | B |
| ATOM | 3747 | CA | GLU | 162 | 32.536 | 16.335 | 63.230 | 1.00 | 10.12 | B |
| ATOM | 3748 | CB | GLU | 162 | 33.914 | 16.845 | 62.829 | 1.00 | 9.47 | B |
| ATOM | 3749 | CG | GLU | 162 | 34.143 | 16.845 | 61.353 | 1.00 | 9.35 | B |
| ATOM | 3750 | CD | GLU | 162 | 35.607 | 16.813 | 61.008 | 1.00 | 9.38 | B |
| ATOM | 3751 | OE1 | GLU | 162 | 36.443 | 17.239 | 61.829 | 1.00 | 9.19 | B |
| ATOM | 3752 | OE2 | GLU | 162 | 35.929 | 16.357 | 59.901 | 1.00 | 8.99 | B |
| ATOM | 3753 | C | GLU | 162 | 32.339 | 16.498 | 64.729 | 1.00 | 10.38 | B |
| ATOM | 3754 | O | GLU | 162 | 31.849 | 17.527 | 65.222 | 1.00 | 7.96 | B |
| ATOM | 3755 | N | ILE | 163 | 32.734 | 15.456 | 65.444 | 1.00 | 10.66 | B |
| ATOM | 3756 | CA | ILE | 163 | 32.581 | 15.414 | 66.879 | 1.00 | 10.98 | B |
| ATOM | 3757 | CB | ILE | 163 | 31.782 | 14.160 | 67.293 | 1.00 | 11.27 | B |
| ATOM | 3758 | CG2 | ILE | 163 | 31.505 | 14.192 | 68.793 | 1.00 | 11.05 | B |
| ATOM | 3759 | CG1 | ILE | 163 | 30.504 | 14.066 | 66.462 | 1.00 | 11.37 | B |
| ATOM | 3760 | CD1 | ILE | 163 | 29.804 | 12.728 | 66.528 | 1.00 | 12.73 | B |
| ATOM | 3761 | C | ILE | 163 | 33.941 | 15.387 | 67.559 | 1.00 | 10.94 | B |
| ATOM | 3762 | O | ILE | 163 | 34.849 | 14.680 | 67.127 | 1.00 | 11.24 | B |
| ATOM | 3763 | N | TYR | 164 | 34.071 | 16.177 | 68.619 | 1.00 | 10.16 | B |
| ATOM | 3764 | CA | TYR | 164 | 35.303 | 16.245 | 69.376 | 1.00 | 8.14 | B |
| ATOM | 3765 | CB | TYR | 164 | 36.254 | 17.270 | 68.759 | 1.00 | 5.82 | B |
| ATOM | 3766 | CG | TYR | 164 | 37.517 | 17.425 | 69.533 | 1.00 | 3.86 | B |
| ATOM | 3767 | CD1 | TYR | 164 | 37.560 | 18.215 | 70.682 | 1.00 | 5.62 | B |
| ATOM | 3768 | CE1 | TYR | 164 | 38.709 | 18.292 | 71.465 | 1.00 | 4.56 | B |
| ATOM | 3769 | CD2 | TYR | 164 | 38.651 | 16.719 | 69.177 | 1.00 | 3.71 | B |
| ATOM | 3770 | CE2 | TYR | 164 | 39.811 | 16.786 | 69.955 | 1.00 | 5.19 | B |
| ATOM | 3771 | CZ | TYR | 164 | 39.827 | 17.577 | 71.094 | 1.00 | 4.77 | B |
| ATOM | 3772 | OH | TYR | 164 | 40.976 | 17.675 | 71.832 | 1.00 | 5.42 | B |
| ATOM | 3773 | C | TYR | 164 | 34.937 | 16.617 | 70.802 | 1.00 | 8.94 | B |
| ATOM | 3774 | O | TYR | 164 | 34.299 | 17.627 | 71.061 | 1.00 | 9.91 | B |
| ATOM | 3775 | N | ASN | 165 | 35.346 | 15.775 | 71.731 | 1.00 | 10.87 | B |
| ATOM | 3776 | CA | ASN | 165 | 35.050 | 16.003 | 73.134 | 1.00 | 12.54 | B |
| ATOM | 3777 | CB | ASN | 165 | 35.847 | 17.192 | 73.674 | 1.00 | 15.11 | B |
| ATOM | 3778 | CG | ASN | 165 | 35.722 | 17.336 | 75.190 | 1.00 | 19.28 | B |
| ATOM | 3779 | OD1 | ASN | 165 | 35.971 | 16.385 | 75.936 | 1.00 | 21.80 | B |
| ATOM | 3780 | ND2 | ASN | 165 | 35.345 | 18.528 | 75.651 | 1.00 | 20.20 | B |
| ATOM | 3781 | C | ASN | 165 | 33.562 | 16.262 | 73.308 | 1.00 | 12.20 | B |
| ATOM | 3782 | O | ASN | 165 | 33.160 | 17.158 | 74.000 | 1.00 | 10.80 | B |
| ATOM | 3783 | N | GLU | 166 | 32.767 | 15.430 | 72.646 | 1.00 | 16.33 | B |
| ATOM | 3784 | CA | GLU | 166 | 31.304 | 15.495 | 72.656 | 1.00 | 18.28 | B |
| ATOM | 3785 | CB | GLU | 166 | 30.739 | 15.101 | 74.031 | 1.00 | 17.10 | B |
| ATOM | 3786 | CG | GLU | 166 | 30.887 | 13.610 | 74.353 | 1.00 | 16.82 | B |
| ATOM | 3787 | CD | GLU | 166 | 30.175 | 12.693 | 73.357 | 1.00 | 16.06 | B |
| ATOM | 3788 | OE1 | GLU | 166 | 28.928 | 12.606 | 73.360 | 1.00 | 13.96 | B |
| ATOM | 3789 | OE2 | GLU | 166 | 30.880 | 12.055 | 72.559 | 1.00 | 15.35 | B |
| ATOM | 3790 | C | GLU | 166 | 30.697 | 16.825 | 72.201 | 1.00 | 19.60 | B |
| ATOM | 3791 | O | GLU | 166 | 29.604 | 17.192 | 72.606 | 1.00 | 19.36 | B |
| ATOM | 3792 | N | GLU | 167 | 31.427 | 17.546 | 71.357 | 1.00 | 21.89 | B |
| ATOM | 3793 | CA | GLU | 167 | 30.956 | 18.818 | 70.823 | 1.00 | 22.41 | B |
| ATOM | 3794 | CB | GLU | 167 | 31.910 | 19.947 | 71.208 | 1.00 | 24.57 | B |
| ATOM | 3795 | CG | GLU | 167 | 31.998 | 20.181 | 72.701 | 1.00 | 28.83 | B |
| ATOM | 3796 | CD | GLU | 167 | 32.847 | 21.376 | 73.044 | 1.00 | 31.70 | B |
| ATOM | 3797 | OE1 | GLU | 167 | 33.985 | 21.472 | 72.521 | 1.00 | 32.58 | B |
| ATOM | 3798 | OE2 | GLU | 167 | 32.373 | 22.214 | 73.840 | 1.00 | 33.47 | B |
| ATOM | 3799 | C | GLU | 167 | 30.874 | 18.683 | 69.314 | 1.00 | 21.24 | B |
| ATOM | 3800 | O | GLU | 167 | 31.689 | 17.997 | 68.700 | 1.00 | 20.64 | B |
| ATOM | 3801 | N | LEU | 168 | 29.879 | 19.328 | 68.717 | 1.00 | 20.17 | B |
| ATOM | 3802 | CA | LEU | 168 | 29.712 | 19.254 | 67.269 | 1.00 | 19.71 | B |
| ATOM | 3803 | CB | LEU | 168 | 28.240 | 19.110 | 66.887 | 1.00 | 19.82 | B |
| ATOM | 3804 | CG | LEU | 168 | 27.430 | 17.954 | 67.457 | 1.00 | 19.46 | B |
| ATOM | 3805 | CD1 | LEU | 168 | 28.198 | 16.653 | 67.320 | 1.00 | 19.39 | B |
| ATOM | 3806 | CD2 | LEU | 168 | 27.113 | 18.236 | 68.903 | 1.00 | 20.70 | B |
| ATOM | 3807 | C | LEU | 168 | 30.251 | 20.477 | 66.524 | 1.00 | 19.80 | B |
| ATOM | 3808 | O | LEU | 168 | 30.055 | 21.611 | 66.939 | 1.00 | 20.40 | B |
| ATOM | 3809 | N | PHE | 169 | 30.928 | 20.229 | 65.411 | 1.00 | 19.38 | B |
| ATOM | 3810 | CA | PHE | 169 | 31.478 | 21.306 | 64.612 | 1.00 | 17.82 | B |
| ATOM | 3811 | CB | PHE | 169 | 33.004 | 21.327 | 64.706 | 1.00 | 17.88 | B |
| ATOM | 3812 | CG | PHE | 169 | 33.513 | 21.530 | 66.097 | 1.00 | 16.09 | B |
| ATOM | 3813 | CD1 | PHE | 169 | 33.737 | 20.445 | 66.928 | 1.00 | 15.76 | B |
| ATOM | 3814 | CD2 | PHE | 169 | 33.695 | 22.810 | 66.600 | 1.00 | 16.92 | B |
| ATOM | 3815 | CE1 | PHE | 169 | 34.130 | 20.621 | 68.235 | 1.00 | 16.10 | B |
| ATOM | 3816 | CE2 | PHE | 169 | 34.090 | 23.001 | 67.907 | 1.00 | 17.09 | B |
| ATOM | 3817 | CZ | PHE | 169 | 34.308 | 21.901 | 68.731 | 1.00 | 16.73 | B |
| ATOM | 3818 | C | PHE | 169 | 31.068 | 21.102 | 63.166 | 1.00 | 18.77 | B |
| ATOM | 3819 | O | PHE | 169 | 30.929 | 19.980 | 62.704 | 1.00 | 18.62 | B |
| ATOM | 3820 | N | ASP | 170 | 30.871 | 22.206 | 62.459 | 1.00 | 20.24 | B |
| ATOM | 3821 | CA | ASP | 170 | 30.476 | 22.171 | 61.055 | 1.00 | 21.83 | B |
| ATOM | 3822 | CB | ASP | 170 | 29.387 | 23.216 | 60.785 | 1.00 | 20.71 | B |
| ATOM | 3823 | CG | ASP | 170 | 28.832 | 23.135 | 59.382 | 1.00 | 22.77 | B |
| ATOM | 3824 | OD1 | ASP | 170 | 29.510 | 22.563 | 58.493 | 1.00 | 23.50 | B |
| ATOM | 3825 | OD2 | ASP | 170 | 27.724 | 23.658 | 59.158 | 1.00 | 24.44 | B |
| ATOM | 3826 | C | ASP | 170 | 31.714 | 22.545 | 60.269 | 1.00 | 22.03 | B |
| ATOM | 3827 | O | ASP | 170 | 32.119 | 23.693 | 60.281 | 1.00 | 23.16 | B |
| ATOM | 3828 | N | LEU | 171 | 32.320 | 21.577 | 59.593 | 1.00 | 21.95 | B |
| ATOM | 3829 | CA | LEU | 171 | 33.514 | 21.878 | 58.828 | 1.00 | 22.12 | B |
| ATOM | 3830 | CB | LEU | 171 | 34.449 | 20.674 | 58.827 | 1.00 | 20.38 | B |
| ATOM | 3831 | CG | LEU | 171 | 35.422 | 20.605 | 60.013 | 1.00 | 21.16 | B |
| ATOM | 3832 | CD1 | LEU | 171 | 36.359 | 21.824 | 60.018 | 1.00 | 20.44 | B |
| ATOM | 3833 | CD2 | LEU | 171 | 34.645 | 20.544 | 61.307 | 1.00 | 18.78 | B |
| ATOM | 3834 | C | LEU | 171 | 33.271 | 22.356 | 57.402 | 1.00 | 24.20 | B |
| ATOM | 3835 | O | LEU | 171 | 34.201 | 22.357 | 56.582 | 1.00 | 24.74 | B |
| ATOM | 3836 | N | LEU | 172 | 32.034 | 22.764 | 57.108 | 1.00 | 26.40 | B |
| ATOM | 3837 | CA | LEU | 172 | 31.686 | 23.266 | 55.776 | 1.00 | 28.39 | B |
| ATOM | 3838 | CB | LEU | 172 | 30.802 | 22.283 | 55.004 | 1.00 | 28.49 | B |
| ATOM | 3839 | CG | LEU | 172 | 31.536 | 21.056 | 54.448 | 1.00 | 29.54 | B |
| ATOM | 3840 | CD1 | LEU | 172 | 30.562 | 20.216 | 53.633 | 1.00 | 30.71 | B |
| ATOM | 3841 | CD2 | LEU | 172 | 32.730 | 21.477 | 53.583 | 1.00 | 28.53 | B |
| ATOM | 3842 | C | LEU | 172 | 30.979 | 24.607 | 55.797 | 1.00 | 28.89 | B |
| ATOM | 3843 | O | LEU | 172 | 30.416 | 25.030 | 54.823 | 1.00 | 30.09 | B |
| ATOM | 3844 | N | ASN | 173 | 31.007 | 25.264 | 56.941 | 1.00 | 31.10 | B |
| ATOM | 3845 | CA | ASN | 173 | 30.403 | 26.580 | 57.043 | 1.00 | 34.00 | B |
| ATOM | 3846 | CB | ASN | 173 | 29.606 | 26.708 | 58.347 | 1.00 | 33.23 | B |
| ATOM | 3847 | CG | ASN | 173 | 28.903 | 28.053 | 58.473 | 1.00 | 32.72 | B |
| ATOM | 3848 | OD1 | ASN | 173 | 28.108 | 28.268 | 59.381 | 1.00 | 33.30 | B |
| ATOM | 3849 | ND2 | ASN | 173 | 29.205 | 28.967 | 57.551 | 1.00 | 31.17 | B |
| ATOM | 3850 | C | ASN | 173 | 31.554 | 27.579 | 56.982 | 1.00 | 35.93 | B |
| ATOM | 3851 | O | ASN | 173 | 32.402 | 27.627 | 57.861 | 1.00 | 35.47 | B |
| ATOM | 3852 | N | PRO | 174 | 31.609 | 28.372 | 55.908 | 1.00 | 38.25 | B |
| ATOM | 3853 | CD | PRO | 174 | 30.799 | 28.283 | 54.681 | 1.00 | 38.57 | B |
| ATOM | 3854 | CA | PRO | 174 | 32.674 | 29.362 | 55.753 | 1.00 | 40.38 | B |
| ATOM | 3855 | CB | PRO | 174 | 32.702 | 29.569 | 54.242 | 1.00 | 39.65 | B |
| ATOM | 3856 | CG | PRO | 174 | 31.264 | 29.478 | 53.900 | 1.00 | 38.79 | B |
| ATOM | 3857 | C | PRO | 174 | 32.445 | 30.632 | 56.582 | 1.00 | 42.95 | B |
| ATOM | 3858 | O | PRO | 174 | 33.356 | 31.450 | 56.743 | 1.00 | 43.55 | B |
| ATOM | 3859 | N | SER | 175 | 31.234 | 30.794 | 57.108 | 1.00 | 45.10 | B |
| ATOM | 3860 | CA | SER | 175 | 30.906 | 31.974 | 57.913 | 1.00 | 47.15 | B |
| ATOM | 3861 | CB | SER | 175 | 29.395 | 32.227 | 57.889 | 1.00 | 47.30 | B |
| ATOM | 3862 | OG | SER | 175 | 28.906 | 32.331 | 56.559 | 1.00 | 49.37 | B |
| ATOM | 3863 | C | SER | 175 | 31.369 | 31.882 | 59.376 | 1.00 | 47.57 | B |
| ATOM | 3864 | O | SER | 175 | 31.800 | 32.872 | 59.970 | 1.00 | 48.25 | B |
| ATOM | 3865 | N | SER | 176 | 31.280 | 30.690 | 59.953 | 1.00 | 47.97 | B |
| ATOM | 3866 | CA | SER | 176 | 31.677 | 30.487 | 61.340 | 1.00 | 47.64 | B |
| ATOM | 3867 | CB | SER | 176 | 30.720 | 29.520 | 62.034 | 1.00 | 46.90 | B |
| ATOM | 3868 | OG | SER | 176 | 30.794 | 28.230 | 61.447 | 1.00 | 46.36 | B |
| ATOM | 3869 | C | SER | 176 | 33.083 | 29.917 | 61.451 | 1.00 | 48.54 | B |
| ATOM | 3870 | O | SER | 176 | 33.650 | 29.434 | 60.484 | 1.00 | 48.78 | B |
| ATOM | 3871 | N | ASP | 177 | 33.646 | 29.989 | 62.648 | 1.00 | 49.43 | B |
| ATOM | 3872 | CA | ASP | 177 | 34.979 | 29.467 | 62.874 | 1.00 | 50.07 | B |
| ATOM | 3873 | CB | ASP | 177 | 35.843 | 30.521 | 63.591 | 1.00 | 51.58 | B |
| ATOM | 3874 | CG | ASP | 177 | 35.342 | 30.852 | 64.996 | 1.00 | 53.37 | B |
| ATOM | 3875 | OD1 | ASP | 177 | 35.948 | 31.723 | 65.658 | 1.00 | 54.70 | B |
| ATOM | 3876 | OD2 | ASP | 177 | 34.353 | 30.246 | 65.452 | 1.00 | 54.61 | B |
| ATOM | 3877 | C | ASP | 177 | 34.880 | 28.160 | 63.669 | 1.00 | 49.81 | B |
| ATOM | 3878 | O | ASP | 177 | 33.833 | 27.830 | 64.235 | 1.00 | 48.89 | B |
| ATOM | 3879 | N | VAL | 178 | 35.980 | 27.422 | 63.707 | 1.00 | 49.42 | B |
| ATOM | 3880 | CA | VAL | 178 | 36.030 | 26.146 | 64.409 | 1.00 | 50.03 | B |
| ATOM | 3881 | CB | VAL | 178 | 37.385 | 25.452 | 64.150 | 1.00 | 50.76 | B |
| ATOM | 3882 | CG1 | VAL | 178 | 37.528 | 25.131 | 62.665 | 1.00 | 49.77 | B |
| ATOM | 3883 | CG2 | VAL | 178 | 38.538 | 26.353 | 64.629 | 1.00 | 50.93 | B |
| ATOM | 3884 | C | VAL | 178 | 35.791 | 26.203 | 65.927 | 1.00 | 49.82 | B |
| ATOM | 3885 | O | VAL | 178 | 35.912 | 25.194 | 66.623 | 1.00 | 50.17 | B |
| ATOM | 3886 | N | SER | 179 | 35.451 | 27.372 | 66.447 | 1.00 | 48.85 | B |
| ATOM | 3887 | CA | SER | 179 | 35.225 | 27.491 | 67.877 | 1.00 | 47.91 | B |
| ATOM | 3888 | CB | SER | 179 | 35.912 | 28.749 | 68.397 | 1.00 | 48.14 | B |
| ATOM | 3889 | OG | SER | 179 | 35.472 | 29.884 | 67.667 | 1.00 | 47.90 | B |
| ATOM | 3890 | C | SER | 179 | 33.739 | 27.541 | 68.211 | 1.00 | 47.46 | B |
| ATOM | 3891 | O | SER | 179 | 33.357 | 27.618 | 69.376 | 1.00 | 47.10 | B |
| ATOM | 3892 | N | GLU | 180 | 32.900 | 27.495 | 67.182 | 1.00 | 46.50 | B |
| ATOM | 3893 | CA | GLU | 180 | 31.458 | 27.542 | 67.383 | 1.00 | 45.18 | B |
| ATOM | 3894 | CB | GLU | 180 | 30.835 | 28.527 | 66.383 | 1.00 | 44.47 | B |
| ATOM | 3895 | CG | GLU | 180 | 31.026 | 29.983 | 66.788 | 1.00 | 44.05 | B |
| ATOM | 3896 | CD | GLU | 180 | 30.595 | 30.971 | 65.724 | 1.00 | 43.63 | B |
| ATOM | 3897 | OE1 | GLU | 180 | 31.354 | 31.176 | 64.751 | 1.00 | 43.67 | B |
| ATOM | 3898 | OE2 | GLU | 180 | 29.495 | 31.542 | 65.860 | 1.00 | 42.55 | B |
| ATOM | 3899 | C | GLU | 180 | 30.813 | 26.156 | 67.295 | 1.00 | 44.60 | B |
| ATOM | 3900 | O | GLU | 180 | 30.714 | 25.570 | 66.228 | 1.00 | 44.37 | B |
| ATOM | 3901 | N | ARG | 181 | 30.373 | 25.650 | 68.445 | 1.00 | 44.01 | B |
| ATOM | 3902 | CA | ARG | 181 | 29.739 | 24.342 | 68.529 | 1.00 | 42.83 | B |
| ATOM | 3903 | CB | ARG | 181 | 29.775 | 23.806 | 69.958 | 1.00 | 45.18 | B |
| ATOM | 3904 | CG | ARG | 181 | 28.755 | 24.439 | 70.895 | 1.00 | 47.37 | B |
| ATOM | 3905 | CD | ARG | 181 | 28.693 | 23.644 | 72.187 | 1.00 | 51.45 | B |
| ATOM | 3906 | NE | ARG | 181 | 27.541 | 23.972 | 73.034 | 1.00 | 54.79 | B |
| ATOM | 3907 | CZ | ARG | 181 | 26.267 | 23.753 | 72.706 | 1.00 | 56.32 | B |
| ATOM | 3908 | NH1 | ARG | 181 | 25.969 | 23.205 | 71.539 | 1.00 | 57.53 | B |
| ATOM | 3909 | NH2 | ARG | 181 | 25.286 | 24.065 | 73.548 | 1.00 | 56.18 | B |
| ATOM | 3910 | C | ARG | 181 | 28.278 | 24.404 | 68.121 | 1.00 | 40.59 | B |
| ATOM | 3911 | O | ARG | 181 | 27.632 | 25.414 | 68.254 | 1.00 | 41.20 | B |
| ATOM | 3912 | N | LEU | 182 | 27.759 | 23.293 | 67.632 | 1.00 | 38.61 | B |
| ATOM | 3913 | CA | LEU | 182 | 26.370 | 23.253 | 67.219 | 1.00 | 35.94 | B |
| ATOM | 3914 | CB | LEU | 182 | 26.259 | 22.490 | 65.897 | 1.00 | 34.47 | B |
| ATOM | 3915 | CG | LEU | 182 | 27.018 | 23.098 | 64.718 | 1.00 | 31.55 | B |
| ATOM | 3916 | CD1 | LEU | 182 | 26.951 | 22.179 | 63.525 | 1.00 | 30.32 | B |
| ATOM | 3917 | CD2 | LEU | 182 | 26.417 | 24.440 | 64.382 | 1.00 | 29.89 | B |
| ATOM | 3918 | C | LEU | 182 | 25.532 | 22.579 | 68.300 | 1.00 | 35.46 | B |
| ATOM | 3919 | O | LEU | 182 | 26.057 | 21.845 | 69.139 | 1.00 | 35.35 | B |
| ATOM | 3920 | N | GLN | 183 | 24.227 | 22.839 | 68.270 | 1.00 | 35.14 | B |
| ATOM | 3921 | CA | GLN | 183 | 23.290 | 22.256 | 69.228 | 1.00 | 33.43 | B |
| ATOM | 3922 | CB | GLN | 183 | 22.261 | 23.284 | 69.688 | 1.00 | 36.19 | B |
| ATOM | 3923 | CG | GLN | 183 | 22.844 | 24.463 | 70.456 | 1.00 | 40.60 | B |
| ATOM | 3924 | CD | GLN | 183 | 21.781 | 25.458 | 70.916 | 1.00 | 43.17 | B |
| ATOM | 3925 | OE1 | GLN | 183 | 20.902 | 25.122 | 71.711 | 1.00 | 45.10 | B |
| ATOM | 3926 | NE2 | GLN | 183 | 21.856 | 26.687 | 70.408 | 1.00 | 42.17 | B |
| ATOM | 3927 | C | GLN | 183 | 22.513 | 21.122 | 68.578 | 1.00 | 30.84 | B |
| ATOM | 3928 | O | GLN | 183 | 22.098 | 21.224 | 67.436 | 1.00 | 29.43 | B |
| ATOM | 3929 | N | MET | 184 | 22.311 | 20.047 | 69.325 | 1.00 | 29.11 | B |
| ATOM | 3930 | CA | MET | 184 | 21.603 | 18.884 | 68.821 | 1.00 | 28.51 | B |
| ATOM | 3931 | CB | MET | 184 | 22.549 | 17.698 | 68.930 | 1.00 | 27.68 | B |
| ATOM | 3932 | CG | MET | 184 | 21.997 | 16.385 | 68.443 | 1.00 | 30.34 | B |
| ATOM | 3933 | SD | MET | 184 | 23.142 | 15.021 | 68.745 | 1.00 | 30.67 | B |
| ATOM | 3934 | CE | MET | 184 | 22.841 | 14.793 | 70.448 | 1.00 | 30.06 | B |
| ATOM | 3935 | C | MET | 184 | 20.298 | 18.650 | 69.595 | 1.00 | 29.09 | B |
| ATOM | 3936 | O | MET | 184 | 20.280 | 18.737 | 70.806 | 1.00 | 29.05 | B |
| ATOM | 3937 | N | PHE | 185 | 19.213 | 18.342 | 68.887 | 1.00 | 30.68 | B |
| ATOM | 3938 | CA | PHE | 185 | 17.921 | 18.112 | 69.537 | 1.00 | 31.83 | B |
| ATOM | 3939 | CB | PHE | 185 | 16.953 | 19.277 | 69.291 | 1.00 | 31.45 | B |
| ATOM | 3940 | CG | PHE | 185 | 17.520 | 20.626 | 69.637 | 1.00 | 30.24 | B |
| ATOM | 3941 | CD1 | PHE | 185 | 18.381 | 21.275 | 68.763 | 1.00 | 29.12 | B |
| ATOM | 3942 | CD2 | PHE | 185 | 17.215 | 21.234 | 70.850 | 1.00 | 28.98 | B |
| ATOM | 3943 | CE1 | PHE | 185 | 18.929 | 22.500 | 69.082 | 1.00 | 28.97 | B |
| ATOM | 3944 | CE2 | PHE | 185 | 17.762 | 22.461 | 71.180 | 1.00 | 29.87 | B |
| ATOM | 3945 | CZ | PHE | 185 | 18.624 | 23.098 | 70.289 | 1.00 | 29.79 | B |
| ATOM | 3946 | C | PHE | 185 | 17.236 | 16.883 | 68.976 | 1.00 | 33.71 | B |
| ATOM | 3947 | O | PHE | 185 | 17.473 | 16.515 | 67.845 | 1.00 | 33.43 | B |
| ATOM | 3948 | N | ASP | 186 | 16.393 | 16.245 | 69.782 | 1.00 | 37.53 | B |
| ATOM | 3949 | CA | ASP | 186 | 15.667 | 15.071 | 69.310 | 1.00 | 40.98 | B |
| ATOM | 3950 | CB | ASP | 186 | 14.857 | 14.413 | 70.431 | 1.00 | 43.17 | B |
| ATOM | 3951 | CG | ASP | 186 | 15.721 | 13.931 | 71.575 | 1.00 | 45.72 | B |
| ATOM | 3952 | OD1 | ASP | 186 | 16.691 | 13.190 | 71.316 | 1.00 | 48.29 | B |
| ATOM | 3953 | OD2 | ASP | 186 | 15.413 | 14.291 | 72.734 | 1.00 | 46.64 | B |
| ATOM | 3954 | C | ASP | 186 | 14.676 | 15.587 | 68.284 | 1.00 | 42.58 | B |
| ATOM | 3955 | O | ASP | 186 | 14.123 | 16.666 | 68.453 | 1.00 | 42.55 | B |
| ATOM | 3956 | N | ASP | 187 | 14.457 | 14.835 | 67.214 | 1.00 | 44.89 | B |
| ATOM | 3957 | CA | ASP | 187 | 13.528 | 15.287 | 66.188 | 1.00 | 46.96 | B |
| ATOM | 3958 | CB | ASP | 187 | 13.921 | 14.695 | 64.840 | 1.00 | 46.66 | B |
| ATOM | 3959 | CG | ASP | 187 | 13.090 | 15.232 | 63.718 | 1.00 | 46.68 | B |
| ATOM | 3960 | OD1 | ASP | 187 | 13.381 | 14.891 | 62.555 | 1.00 | 47.95 | B |
| ATOM | 3961 | OD2 | ASP | 187 | 12.144 | 15.996 | 64.008 | 1.00 | 45.37 | B |
| ATOM | 3962 | C | ASP | 187 | 12.127 | 14.881 | 66.604 | 1.00 | 48.78 | B |
| ATOM | 3963 | O | ASP | 187 | 11.844 | 13.696 | 66.773 | 1.00 | 49.04 | B |
| ATOM | 3964 | N | PRO | 188 | 11.235 | 15.870 | 66.799 | 1.00 | 50.85 | B |
| ATOM | 3965 | CD | PRO | 188 | 11.546 | 17.310 | 66.716 | 1.00 | 50.78 | B |
| ATOM | 3966 | CA | PRO | 188 | 9.838 | 15.660 | 67.209 | 1.00 | 52.07 | B |
| ATOM | 3967 | CB | PRO | 188 | 9.280 | 17.085 | 67.240 | 1.00 | 51.41 | B |
| ATOM | 3968 | CG | PRO | 188 | 10.496 | 17.916 | 67.605 | 1.00 | 50.84 | B |
| ATOM | 3969 | C | PRO | 188 | 9.071 | 14.705 | 66.302 | 1.00 | 53.79 | B |
| ATOM | 3970 | O | PRO | 188 | 8.249 | 13.900 | 66.753 | 1.00 | 52.56 | B |
| ATOM | 3971 | N | ARG | 189 | 9.340 | 14.817 | 65.011 | 1.00 | 56.26 | B |
| ATOM | 3972 | CA | ARG | 189 | 8.691 | 13.979 | 64.033 | 1.00 | 59.28 | B |
| ATOM | 3973 | CB | ARG | 189 | 9.218 | 14.349 | 62.649 | 1.00 | 60.03 | B |
| ATOM | 3974 | CG | ARG | 189 | 8.875 | 15.774 | 62.238 | 1.00 | 61.54 | B |
| ATOM | 3975 | CD | ARG | 189 | 9.366 | 16.081 | 60.833 | 1.00 | 62.62 | B |
| ATOM | 3976 | NE | ARG | 189 | 10.813 | 16.277 | 60.790 | 1.00 | 63.59 | B |
| ATOM | 3977 | CZ | ARG | 189 | 11.407 | 17.465 | 60.837 | 1.00 | 64.36 | B |
| ATOM | 3978 | NH1 | ARG | 189 | 10.680 | 18.575 | 60.925 | 1.00 | 64.67 | B |
| ATOM | 3979 | NH2 | ARG | 189 | 12.729 | 17.545 | 60.794 | 1.00 | 64.73 | B |
| ATOM | 3980 | C | ARG | 189 | 8.905 | 12.499 | 64.357 | 1.00 | 61.00 | B |
| ATOM | 3981 | O | ARG | 189 | 7.952 | 11.725 | 64.399 | 1.00 | 61.27 | B |
| ATOM | 3982 | N | ASN | 190 | 10.159 | 12.118 | 64.590 | 1.00 | 63.40 | B |
| ATOM | 3983 | CA | ASN | 190 | 10.516 | 10.735 | 64.914 | 1.00 | 65.21 | B |
| ATOM | 3984 | CB | ASN | 190 | 10.752 | 9.935 | 63.625 | 1.00 | 65.05 | B |
| ATOM | 3985 | CG | ASN | 190 | 11.750 | 10.604 | 62.692 | 1.00 | 64.67 | B |
| ATOM | 3986 | OD1 | ASN | 190 | 12.954 | 10.474 | 62.861 | 1.00 | 64.77 | B |
| ATOM | 3987 | ND2 | ASN | 190 | 11.242 | 11.332 | 61.707 | 1.00 | 63.52 | B |
| ATOM | 3988 | C | ASN | 190 | 11.757 | 10.684 | 65.807 | 1.00 | 66.41 | B |
| ATOM | 3989 | O | ASN | 190 | 12.850 | 11.038 | 65.381 | 1.00 | 66.57 | B |
| ATOM | 3990 | N | LYS | 191 | 11.575 | 10.241 | 67.051 | 1.00 | 67.89 | B |
| ATOM | 3991 | CA | LYS | 191 | 12.676 | 10.158 | 68.017 | 1.00 | 68.02 | B |
| ATOM | 3992 | CB | LYS | 191 | 12.151 | 9.687 | 69.378 | 1.00 | 69.77 | B |
| ATOM | 3993 | CG | LYS | 191 | 11.151 | 10.636 | 70.012 | 1.00 | 71.09 | B |
| ATOM | 3994 | CD | LYS | 191 | 11.787 | 11.982 | 70.297 | 1.00 | 72.77 | B |
| ATOM | 3995 | CE | LYS | 191 | 10.771 | 12.963 | 70.860 | 1.00 | 74.00 | B |
| ATOM | 3996 | NZ | LYS | 191 | 9.657 | 13.210 | 69.902 | 1.00 | 75.27 | B |
| ATOM | 3997 | C | LYS | 191 | 13.826 | 9.251 | 67.571 | 1.00 | 66.64 | B |
| ATOM | 3998 | O | LYS | 191 | 14.852 | 9.149 | 68.253 | 1.00 | 66.18 | B |
| ATOM | 3999 | N | ARG | 192 | 13.641 | 8.587 | 66.434 | 1.00 | 64.41 | B |
| ATOM | 4000 | CA | ARG | 192 | 14.668 | 7.720 | 65.878 | 1.00 | 62.32 | B |
| ATOM | 4001 | CB | ARG | 192 | 14.101 | 6.946 | 64.685 | 1.00 | 64.84 | B |
| ATOM | 4002 | CG | ARG | 192 | 15.134 | 6.138 | 63.909 | 1.00 | 68.49 | B |
| ATOM | 4003 | CD | ARG | 192 | 14.582 | 5.584 | 62.578 | 1.00 | 71.52 | B |
| ATOM | 4004 | NE | ARG | 192 | 14.312 | 6.616 | 61.569 | 1.00 | 73.79 | B |
| ATOM | 4005 | CZ | ARG | 192 | 13.207 | 7.359 | 61.506 | 1.00 | 74.82 | B |
| ATOM | 4006 | NE1 | ARG | 192 | 12.232 | 7.201 | 62.393 | 1.00 | 75.36 | B |
| ATOM | 4007 | NH2 | ARG | 192 | 13.079 | 8.275 | 60.555 | 1.00 | 75.53 | B |
| ATOM | 4008 | C | ARG | 192 | 15.822 | 8.612 | 65.403 | 1.00 | 59.33 | B |
| ATOM | 4009 | O | ARG | 192 | 16.991 | 8.235 | 65.479 | 1.00 | 58.48 | B |
| ATOM | 4010 | N | GLY | 193 | 15.468 | 9.805 | 64.927 | 1.00 | 55.93 | B |
| ATOM | 4011 | CA | GLY | 193 | 16.453 | 10.747 | 64.429 | 1.00 | 50.05 | B |
| ATOM | 4012 | C | GLY | 193 | 16.778 | 11.895 | 65.364 | 1.00 | 45.96 | B |
| ATOM | 4013 | O | GLY | 193 | 16.345 | 11.933 | 66.518 | 1.00 | 44.90 | B |
| ATOM | 4014 | N | VAL | 194 | 17.547 | 12.842 | 64.839 | 1.00 | 42.75 | B |
| ATOM | 4015 | CA | VAL | 194 | 17.968 | 14.006 | 65.596 | 1.00 | 39.18 | B |
| ATOM | 4016 | CB | VAL | 194 | 19.328 | 13.743 | 66.269 | 1.00 | 39.02 | B |
| ATOM | 4017 | CG1 | VAL | 194 | 20.450 | 13.925 | 65.262 | 1.00 | 38.70 | B |
| ATOM | 4018 | CG2 | VAL | 194 | 19.504 | 14.653 | 67.456 | 1.00 | 38.46 | B |
| ATOM | 4019 | C | VAL | 194 | 18.096 | 15.209 | 64.666 | 1.00 | 37.27 | B |
| ATOM | 4020 | O | VAL | 194 | 18.181 | 15.057 | 63.456 | 1.00 | 36.48 | B |
| ATOM | 4021 | N | ILE | 195 | 18.108 | 16.400 | 65.254 | 1.00 | 35.15 | B |
| ATOM | 4022 | CA | ILE | 195 | 18.230 | 17.645 | 64.501 | 1.00 | 33.17 | B |
| ATOM | 4023 | CB | ILE | 195 | 17.002 | 18.543 | 64.702 | 1.00 | 34.99 | B |
| ATOM | 4024 | CG2 | ILE | 195 | 17.185 | 19.842 | 63.916 | 1.00 | 36.47 | B |
| ATOM | 4025 | CG1 | ILE | 195 | 15.731 | 17.803 | 64.280 | 1.00 | 36.88 | B |
| ATOM | 4026 | CD1 | ILE | 195 | 15.658 | 17.513 | 62.784 | 1.00 | 38.32 | B |
| ATOM | 4027 | C | ILE | 195 | 19.452 | 18.465 | 64.917 | 1.00 | 30.37 | B |
| ATOM | 4028 | O | ILE | 195 | 19.575 | 18.870 | 66.063 | 1.00 | 28.47 | B |
| ATOM | 4029 | N | ILE | 196 | 20.353 | 18.711 | 63.975 | 1.00 | 28.58 | B |
| ATOM | 4030 | CA | ILE | 196 | 21.538 | 19.503 | 64.270 | 1.00 | 27.51 | B |
| ATOM | 4031 | CB | ILE | 196 | 22.810 | 18.928 | 63.572 | 1.00 | 26.71 | B |
| ATOM | 4032 | CG2 | ILE | 196 | 24.024 | 19.795 | 63.884 | 1.00 | 25.48 | B |
| ATOM | 4033 | CG1 | ILE | 196 | 23.107 | 17.515 | 64.078 | 1.00 | 25.19 | B |
| ATOM | 4034 | CD1 | ILE | 196 | 22.263 | 16.456 | 63.472 | 1.00 | 25.37 | B |
| ATOM | 4035 | C | ILE | 196 | 21.284 | 20.931 | 63.787 | 1.00 | 27.55 | B |
| ATOM | 4036 | O | ILE | 196 | 21.307 | 21.212 | 62.601 | 1.00 | 27.49 | B |
| ATOM | 4037 | N | LYS | 197 | 21.045 | 21.832 | 64.730 | 1.00 | 28.27 | B |
| ATOM | 4038 | CA | LYS | 197 | 20.765 | 23.229 | 64.418 | 1.00 | 27.24 | B |
| ATOM | 4039 | CB | LYS | 197 | 20.328 | 23.973 | 65.688 | 1.00 | 28.18 | B |
| ATOM | 4040 | CG | LYS | 197 | 19.970 | 25.451 | 65.508 | 1.00 | 26.93 | B |
| ATOM | 4041 | CD | LYS | 197 | 19.665 | 26.075 | 66.853 | 1.00 | 27.21 | B |
| ATOM | 4042 | CE | LYS | 197 | 19.417 | 27.563 | 66.750 | 1.00 | 26.28 | B |
| ATOM | 4043 | NZ | LYS | 197 | 19.153 | 28.144 | 68.104 | 1.00 | 26.63 | B |
| ATOM | 4044 | C | LYS | 197 | 21.961 | 23.947 | 63.821 | 1.00 | 26.61 | B |
| ATOM | 4045 | O | LYS | 197 | 23.039 | 23.974 | 64.406 | 1.00 | 27.65 | B |
| ATOM | 4046 | N | GLY | 198 | 21.762 | 24.513 | 62.637 | 1.00 | 26.31 | B |
| ATOM | 4047 | CA | GLY | 198 | 22.826 | 25.266 | 61.998 | 1.00 | 25.56 | B |
| ATOM | 4048 | C | GLY | 198 | 23.747 | 24.536 | 61.044 | 1.00 | 24.60 | B |
| ATOM | 4049 | O | GLY | 198 | 24.518 | 25.162 | 60.335 | 1.00 | 24.69 | B |
| ATOM | 4050 | N | LEU | 199 | 23.680 | 23.211 | 61.029 | 1.00 | 25.09 | B |
| ATOM | 4051 | CA | LEU | 199 | 24.523 | 22.433 | 60.130 | 1.00 | 25.50 | B |
| ATOM | 4052 | CB | LEU | 199 | 24.357 | 20.927 | 60.411 | 1.00 | 24.64 | B |
| ATOM | 4053 | CG | LEU | 199 | 25.219 | 19.950 | 59.597 | 1.00 | 24.37 | B |
| ATOM | 4054 | CD1 | LEU | 199 | 26.699 | 20.274 | 59.742 | 1.00 | 22.90 | B |
| ATOM | 4055 | CD2 | LEU | 199 | 24.942 | 18.535 | 60.068 | 1.00 | 23.77 | B |
| ATOM | 4056 | C | LEU | 199 | 24.235 | 22.767 | 58.648 | 1.00 | 25.50 | B |
| ATOM | 4057 | O | LEU | 199 | 23.160 | 22.510 | 58.114 | 1.00 | 24.77 | B |
| ATOM | 4058 | N | LEU | 200 | 25.225 | 23.350 | 57.991 | 1.00 | 26.00 | B |
| ATOM | 4059 | CA | GLU | 200 | 25.087 | 23.722 | 56.598 | 1.00 | 26.47 | B |
| ATOM | 4060 | CB | GLU | 200 | 26.274 | 24.568 | 56.143 | 1.00 | 27.75 | B |
| ATOM | 4061 | CG | GLU | 200 | 26.324 | 25.971 | 56.724 | 1.00 | 32.47 | B |
| ATOM | 4062 | CD | GLU | 200 | 25.112 | 26.821 | 56.339 | 1.00 | 35.25 | B |
| ATOM | 4063 | OE1 | GLU | 200 | 24.061 | 26.700 | 57.004 | 1.00 | 38.07 | B |
| ATOM | 4064 | OE2 | GLU | 200 | 25.196 | 27.600 | 55.363 | 1.00 | 35.41 | B |
| ATOM | 4065 | C | GLU | 200 | 25.029 | 22.508 | 55.686 | 1.00 | 27.12 | B |
| ATOM | 4066 | O | GLU | 200 | 25.586 | 21.457 | 55.972 | 1.00 | 26.69 | B |
| ATOM | 4067 | N | GLU | 201 | 24.327 | 22.678 | 54.579 | 1.00 | 27.51 | B |
| ATOM | 4068 | CA | GLU | 201 | 24.218 | 21.646 | 53.574 | 1.00 | 26.72 | B |
| ATOM | 4069 | CB | GLU | 201 | 22.790 | 21.135 | 53.468 | 1.00 | 27.33 | B |
| ATOM | 4070 | CG | GLU | 201 | 22.239 | 20.532 | 54.722 | 1.00 | 30.03 | B |
| ATOM | 4071 | CD | GLU | 201 | 20.954 | 19.773 | 54.457 | 1.00 | 32.95 | B |
| ATOM | 4072 | OE1 | GLU | 201 | 20.075 | 19.784 | 55.345 | 1.00 | 34.01 | B |
| ATOM | 4073 | OE2 | GLU | 201 | 20.817 | 19.167 | 53.367 | 1.00 | 33.38 | B |
| ATOM | 4074 | C | GLU | 201 | 24.581 | 22.363 | 52.278 | 1.00 | 26.18 | B |
| ATOM | 4075 | O | GLU | 201 | 23.866 | 23.259 | 51.853 | 1.00 | 25.94 | B |
| ATOM | 4076 | N | ILE | 202 | 25.707 | 21.996 | 51.674 | 1.00 | 25.78 | B |
| ATOM | 4077 | CA | ILE | 202 | 26.116 | 22.631 | 50.433 | 1.00 | 25.80 | B |
| ATOM | 4078 | CB | ILE | 202 | 27.636 | 22.813 | 50.360 | 1.00 | 25.61 | B |
| ATOM | 4079 | CG2 | ILE | 202 | 28.022 | 23.102 | 48.914 | 1.00 | 25.19 | B |
| ATOM | 4080 | CG1 | ILE | 202 | 28.089 | 23.969 | 51.258 | 1.00 | 26.32 | B |
| ATOM | 4081 | CD1 | ILE | 202 | 27.704 | 23.871 | 52.722 | 1.00 | 25.98 | B |
| ATOM | 4082 | C | ILE | 202 | 25.655 | 21.820 | 49.231 | 1.00 | 26.76 | B |
| ATOM | 4083 | O | ILE | 202 | 25.798 | 20.597 | 49.195 | 1.00 | 26.87 | B |
| ATOM | 4084 | N | THR | 203 | 25.089 | 22.508 | 48.248 | 1.00 | 26.89 | B |
| ATOM | 4085 | CA | THR | 203 | 24.610 | 21.817 | 47.070 | 1.00 | 28.63 | B |
| ATOM | 4086 | CB | THR | 203 | 23.463 | 22.606 | 46.329 | 1.00 | 28.93 | B |
| ATOM | 4087 | OG1 | THR | 203 | 22.297 | 22.683 | 47.167 | 1.00 | 28.96 | B |
| ATOM | 4088 | CG2 | THR | 203 | 23.103 | 21.922 | 44.987 | 1.00 | 25.61 | B |
| ATOM | 4089 | C | THR | 203 | 25.774 | 21.634 | 46.120 | 1.00 | 29.69 | B |
| ATOM | 4090 | O | THR | 203 | 26.546 | 22.547 | 45.906 | 1.00 | 31.36 | B |
| ATOM | 4091 | N | VAL | 204 | 25.919 | 20.428 | 45.589 | 1.00 | 30.40 | B |
| ATOM | 4092 | CA | VAL | 204 | 26.967 | 20.168 | 44.620 | 1.00 | 30.44 | B |
| ATOM | 4093 | CB | VAL | 204 | 27.656 | 18.798 | 44.876 | 1.00 | 29.19 | B |
| ATOM | 4094 | CG1 | VAL | 204 | 28.839 | 18.609 | 43.930 | 1.00 | 28.81 | B |
| ATOM | 4095 | CG2 | VAL | 204 | 28.142 | 18.733 | 46.292 | 1.00 | 29.07 | B |
| ATOM | 4096 | C | VAL | 204 | 26.225 | 20.159 | 43.277 | 1.00 | 31.43 | B |
| ATOM | 4097 | O | VAL | 204 | 25.536 | 19.180 | 42.956 | 1.00 | 31.70 | B |
| ATOM | 4098 | N | HIS | 205 | 26.354 | 21.255 | 42.521 | 1.00 | 31.11 | B |
| ATOM | 4099 | CA | HIS | 205 | 25.709 | 21.420 | 41.214 | 1.00 | 30.37 | B |
| ATOM | 4100 | CB | HIS | 205 | 25.803 | 22.869 | 40.792 | 1.00 | 29.29 | B |
| ATOM | 4101 | CG | HIS | 205 | 25.131 | 23.788 | 41.747 | 1.00 | 29.35 | B |
| ATOM | 4102 | CD2 | HIS | 205 | 25.631 | 24.594 | 42.712 | 1.00 | 29.07 | B |
| ATOM | 4103 | ND1 | HIS | 205 | 23.760 | 23.890 | 41.831 | 1.00 | 29.17 | B |
| ATOM | 4104 | CE1 | HIS | 205 | 23.444 | 24.721 | 42.806 | 1.00 | 29.14 | B |
| ATOM | 4105 | NE2 | HIS | 205 | 24.561 | 25.161 | 43.357 | 1.00 | 29.64 | B |
| ATOM | 4106 | C | HIS | 205 | 26.252 | 20.533 | 40.100 | 1.00 | 30.88 | B |
| ATOM | 4107 | O | HIS | 205 | 25.508 | 20.130 | 39.216 | 1.00 | 31.82 | B |
| ATOM | 4108 | N | ASN | 206 | 27.544 | 20.238 | 40.138 | 1.00 | 29.74 | B |
| ATOM | 4109 | CA | ASN | 206 | 28.127 | 19.370 | 39.141 | 1.00 | 29.11 | B |
| ATOM | 4110 | CB | ASN | 206 | 28.377 | 20.158 | 37.852 | 1.00 | 28.48 | B |
| ATOM | 4111 | CG | ASN | 206 | 29.156 | 21.438 | 38.091 | 1.00 | 29.29 | B |
| ATOM | 4112 | OD1 | ASN | 206 | 30.252 | 21.412 | 38.645 | 1.00 | 28.71 | B |
| ATOM | 4113 | ND2 | ASN | 206 | 28.594 | 22.562 | 37.673 | 1.00 | 28.54 | B |
| ATOM | 4114 | C | ASN | 206 | 29.387 | 18.760 | 39.729 | 1.00 | 28.47 | B |
| ATOM | 4115 | O | ASN | 206 | 29.740 | 19.032 | 40.852 | 1.00 | 27.98 | B |
| ATOM | 4116 | N | LYS | 207 | 30.063 | 17.924 | 38.957 | 1.00 | 29.11 | B |
| ATOM | 4117 | CA | LYS | 207 | 31.274 | 17.291 | 39.445 | 1.00 | 30.00 | B |
| ATOM | 4118 | CB | LYS | 207 | 31.662 | 16.107 | 38.553 | 1.00 | 30.11 | B |
| ATOM | 4119 | CG | LYS | 207 | 32.257 | 16.495 | 37.222 | 1.00 | 32.75 | B |
| ATOM | 4120 | CD | LYS | 207 | 32.719 | 15.270 | 36.441 | 1.00 | 33.95 | B |
| ATOM | 4121 | CE | LYS | 207 | 33.466 | 15.669 | 35.164 | 1.00 | 34.56 | B |
| ATOM | 4122 | NZ | LYS | 207 | 34.775 | 16.370 | 35.404 | 1.00 | 33.30 | B |
| ATOM | 4123 | C | LYS | 207 | 32.425 | 18.293 | 39.488 | 1.00 | 30.73 | B |
| ATOM | 4124 | O | LYS | 207 | 33.458 | 18.026 | 40.089 | 1.00 | 32.12 | B |
| ATOM | 4125 | N | ASP | 208 | 32.241 | 19.451 | 38.863 | 1.00 | 29.02 | B |
| ATOM | 4126 | CA | ASP | 208 | 33.301 | 20.453 | 38.850 | 1.00 | 28.26 | B |
| ATOM | 4127 | CB | ASP | 208 | 33.234 | 21.261 | 37.556 | 1.00 | 31.08 | B |
| ATOM | 4128 | CG | ASP | 208 | 33.702 | 20.463 | 36.354 | 1.00 | 32.65 | B |
| ATOM | 4129 | OD1 | ASP | 208 | 33.221 | 20.729 | 35.233 | 1.00 | 33.84 | B |
| ATOM | 4130 | OD2 | ASP | 208 | 34.567 | 19.570 | 36.523 | 1.00 | 33.75 | B |
| ATOM | 4131 | C | ASP | 208 | 33.277 | 21.374 | 40.065 | 1.00 | 26.42 | B |
| ATOM | 4132 | O | ASP | 208 | 33.989 | 22.372 | 40.117 | 1.00 | 24.98 | B |
| ATOM | 4133 | N | GLU | 209 | 32.462 | 21.032 | 41.052 | 1.00 | 25.24 | B |
| ATOM | 4134 | CA | GLU | 209 | 32.388 | 21.831 | 42.272 | 1.00 | 25.22 | B |
| ATOM | 4135 | CB | GLU | 209 | 30.958 | 22.278 | 42.595 | 1.00 | 27.01 | B |
| ATOM | 4136 | CG | GLU | 209 | 30.306 | 23.237 | 41.602 | 1.00 | 30.48 | B |
| ATOM | 4137 | CD | GLU | 209 | 29.069 | 23.926 | 42.167 | 1.00 | 32.55 | B |
| ATOM | 4138 | OE1 | GLU | 209 | 28.371 | 24.610 | 41.385 | 1.00 | 34.80 | B |
| ATOM | 4139 | OE2 | GLU | 209 | 28.804 | 23.793 | 43.382 | 1.00 | 33.17 | B |
| ATOM | 4140 | C | GLU | 209 | 32.832 | 21.030 | 43.490 | 1.00 | 24.23 | B |
| ATOM | 4141 | O | GLU | 209 | 33.194 | 21.596 | 44.513 | 1.00 | 25.15 | B |
| ATOM | 4142 | N | VAL | 210 | 32.835 | 19.708 | 43.373 | 1.00 | 21.99 | B |
| ATOM | 4143 | CA | VAL | 210 | 33.205 | 18.882 | 44.514 | 1.00 | 18.98 | B |
| ATOM | 4144 | CB | VAL | 210 | 32.987 | 17.360 | 44.217 | 1.00 | 17.62 | B |
| ATOM | 4145 | CG1 | VAL | 210 | 32.238 | 17.180 | 42.928 | 1.00 | 17.92 | B |
| ATOM | 4146 | CG2 | VAL | 210 | 34.290 | 16.638 | 44.159 | 1.00 | 17.49 | B |
| ATOM | 4147 | C | VAL | 210 | 34.609 | 19.093 | 45.082 | 1.00 | 18.13 | B |
| ATOM | 4148 | O | VAL | 210 | 34.775 | 19.138 | 46.289 | 1.00 | 19.29 | B |
| ATOM | 4149 | N | TYR | 211 | 35.620 | 19.238 | 44.232 | 1.00 | 17.72 | B |
| ATOM | 4150 | CA | TYR | 211 | 36.968 | 19.401 | 44.770 | 1.00 | 15.84 | B |
| ATOM | 4151 | CB | TYR | 211 | 38.030 | 19.361 | 43.656 | 1.00 | 14.23 | B |
| ATOM | 4152 | CG | TYR | 211 | 39.441 | 19.224 | 44.196 | 1.00 | 13.57 | B |
| ATOM | 4153 | CD1 | TYR | 211 | 39.807 | 18.110 | 44.937 | 1.00 | 12.81 | B |
| ATOM | 4154 | CE1 | TYR | 211 | 41.062 | 18.018 | 45.528 | 1.00 | 12.54 | B |
| ATOM | 4155 | CD2 | TYR | 211 | 40.379 | 20.246 | 44.048 | 1.00 | 14.65 | B |
| ATOM | 4156 | CE2 | TYR | 211 | 41.651 | 20.166 | 44.642 | 1.00 | 13.74 | B |
| ATOM | 4157 | CZ | TYR | 211 | 41.987 | 19.048 | 45.386 | 1.00 | 14.45 | B |
| ATOM | 4158 | OH | TYR | 211 | 43.235 | 18.972 | 45.997 | 1.00 | 10.15 | B |
| ATOM | 4159 | C | TYR | 211 | 37.083 | 20.665 | 45.608 | 1.00 | 15.70 | B |
| ATOM | 4160 | O | TYR | 211 | 37.626 | 20.620 | 46.696 | 1.00 | 14.92 | B |
| ATOM | 4161 | N | GLN | 212 | 36.557 | 21.781 | 45.101 | 1.00 | 17.75 | B |
| ATOM | 4162 | CA | GLN | 212 | 36.582 | 23.064 | 45.819 | 1.00 | 18.64 | B |
| ATOM | 4163 | CB | GLN | 212 | 35.897 | 24.154 | 44.983 | 1.00 | 19.40 | B |
| ATOM | 4164 | CG | GLN | 212 | 35.962 | 25.543 | 45.607 | 1.00 | 24.51 | B |
| ATOM | 4165 | CD | GLN | 212 | 35.764 | 26.672 | 44.587 | 1.00 | 26.82 | B |
| ATOM | 4166 | OE1 | GLN | 212 | 35.046 | 26.508 | 43.594 | 1.00 | 25.33 | B |
| ATOM | 4167 | NE2 | GLN | 212 | 36.391 | 27.832 | 44.844 | 1.00 | 26.86 | B |
| ATOM | 4168 | C | GLN | 212 | 35.909 | 22.923 | 47.192 | 1.00 | 18.53 | B |
| ATOM | 4169 | O | GLN | 212 | 36.420 | 23.374 | 48.193 | 1.00 | 19.69 | B |
| ATOM | 4170 | N | ILE | 213 | 34.759 | 22.265 | 47.230 | 1.00 | 19.83 | B |
| ATOM | 4171 | CA | ILE | 213 | 34.031 | 22.048 | 48.485 | 1.00 | 19.97 | B |
| ATOM | 4172 | CB | ILE | 213 | 32.664 | 21.350 | 48.237 | 1.00 | 20.59 | B |
| ATOM | 4173 | CG2 | ILE | 213 | 32.022 | 20.933 | 49.579 | 1.00 | 19.77 | B |
| ATOM | 4174 | CG1 | ILE | 213 | 31.758 | 22.285 | 47.441 | 1.00 | 20.66 | B |
| ATOM | 4175 | CD1 | ILE | 213 | 30.505 | 21.626 | 46.928 | 1.00 | 22.87 | B |
| ATOM | 4176 | C | ILE | 213 | 34.831 | 21.189 | 49.461 | 1.00 | 20.10 | B |
| ATOM | 4177 | O | ILE | 213 | 34.822 | 21.446 | 50.672 | 1.00 | 20.46 | B |
| ATOM | 4178 | N | LEU | 214 | 35.489 | 20.156 | 48.937 | 1.00 | 19.00 | B |
| ATOM | 4179 | CA | LEU | 214 | 36.310 | 19.282 | 49.759 | 1.00 | 18.96 | B |
| ATOM | 4180 | CB | LEU | 214 | 36.829 | 18.100 | 48.950 | 1.00 | 18.27 | B |
| ATOM | 4181 | CG | LEU | 214 | 36.013 | 16.826 | 49.015 | 1.00 | 18.28 | B |
| ATOM | 4182 | CD1 | LEU | 214 | 34.547 | 17.179 | 48.926 | 1.00 | 22.38 | B |
| ATOM | 4183 | CD2 | LEU | 214 | 36.443 | 15.908 | 47.895 | 1.00 | 17.95 | B |
| ATOM | 4184 | C | LEU | 214 | 37.507 | 20.048 | 50.316 | 1.00 | 19.17 | B |
| ATOM | 4185 | O | LEU | 214 | 37.920 | 19.821 | 51.443 | 1.00 | 20.21 | B |
| ATOM | 4186 | N | GLU | 215 | 38.055 | 20.967 | 49.523 | 1.00 | 19.88 | B |
| ATOM | 4187 | CA | GLU | 215 | 39.208 | 21.768 | 49.953 | 1.00 | 19.18 | B |
| ATOM | 4188 | CB | GLU | 215 | 39.748 | 22.628 | 48.797 | 1.00 | 19.26 | B |
| ATOM | 4189 | CG | GLU | 215 | 40.496 | 21.863 | 47.699 | 1.00 | 20.08 | B |
| ATOM | 4190 | CD | GLU | 215 | 41.103 | 22.786 | 46.630 | 1.00 | 20.78 | B |
| ATOM | 4191 | OE1 | GLU | 215 | 42.352 | 22.898 | 46.580 | 1.00 | 16.87 | B |
| ATOM | 4192 | OE2 | GLU | 215 | 40.337 | 23.399 | 45.842 | 1.00 | 19.38 | B |
| ATOM | 4193 | C | GLU | 215 | 38.855 | 22.700 | 51.110 | 1.00 | 18.78 | B |
| ATOM | 4194 | O | GLU | 215 | 39.592 | 22.798 | 52.092 | 1.00 | 17.36 | B |
| ATOM | 4195 | N | LYS | 216 | 37.732 | 23.397 | 50.988 | 1.00 | 19.53 | B |
| ATOM | 4196 | CA | LYS | 216 | 37.293 | 24.300 | 52.042 | 1.00 | 20.63 | B |
| ATOM | 4197 | CB | LYS | 216 | 35.993 | 24.988 | 51.620 | 1.00 | 22.77 | B |
| ATOM | 4198 | CG | LYS | 216 | 36.240 | 26.094 | 50.602 | 1.00 | 29.39 | B |
| ATOM | 4199 | CD | LYS | 216 | 34.962 | 26.743 | 50.069 | 1.00 | 33.26 | B |
| ATOM | 4200 | CE | LYS | 216 | 35.281 | 27.963 | 49.187 | 1.00 | 35.91 | B |
| ATOM | 4201 | NZ | LYS | 216 | 36.198 | 27.671 | 48.028 | 1.00 | 37.67 | B |
| ATOM | 4202 | C | LYS | 216 | 37.144 | 23.547 | 53.361 | 1.00 | 20.03 | B |
| ATOM | 4203 | O | LYS | 216 | 37.501 | 24.057 | 54.416 | 1.00 | 21.40 | B |
| ATOM | 4204 | N | GLY | 217 | 36.628 | 22.329 | 53.309 | 1.00 | 18.86 | B |
| ATOM | 4205 | CA | GLY | 217 | 36.492 | 21.587 | 54.543 | 1.00 | 18.29 | B |
| ATOM | 4206 | C | GLY | 217 | 37.869 | 21.334 | 55.128 | 1.00 | 18.39 | B |
| ATOM | 4207 | O | GLY | 217 | 38.103 | 21.531 | 56.307 | 1.00 | 18.74 | B |
| ATOM | 4208 | N | ALA | 218 | 38.792 | 20.895 | 54.282 | 1.00 | 19.27 | B |
| ATOM | 4209 | CA | ALA | 218 | 40.148 | 20.607 | 54.737 | 1.00 | 19.03 | B |
| ATOM | 4210 | CB | ALA | 218 | 40.996 | 20.061 | 53.580 | 1.00 | 18.52 | B |
| ATOM | 4211 | C | ALA | 218 | 40.827 | 21.818 | 55.363 | 1.00 | 18.17 | B |
| ATOM | 4212 | O | ALA | 218 | 41.470 | 21.706 | 56.403 | 1.00 | 19.12 | B |
| ATOM | 4213 | N | ALA | 219 | 40.691 | 22.980 | 54.735 | 1.00 | 17.99 | B |
| ATOM | 4214 | CA | ALA | 219 | 41.315 | 24.203 | 55.266 | 1.00 | 16.17 | B |
| ATOM | 4215 | CB | ALA | 219 | 41.044 | 25.404 | 54.323 | 1.00 | 14.07 | B |
| ATOM | 4216 | C | ALA | 219 | 40.792 | 24.505 | 56.671 | 1.00 | 14.78 | B |
| ATOM | 4217 | O | ALA | 219 | 41.552 | 24.760 | 57.599 | 1.00 | 15.56 | B |
| ATOM | 4218 | N | LYS | 220 | 39.479 | 24.450 | 56.823 | 1.00 | 14.00 | B |
| ATOM | 4219 | CA | LYS | 220 | 38.859 | 24.729 | 58.110 | 1.00 | 13.80 | B |
| ATOM | 4220 | CB | LYS | 220 | 37.338 | 24.667 | 57.978 | 1.00 | 11.84 | B |
| ATOM | 4221 | CG | LYS | 220 | 36.603 | 25.222 | 59.177 | 1.00 | 12.63 | B |
| ATOM | 4222 | CD | LYS | 220 | 35.130 | 25.462 | 58.884 | 1.00 | 11.67 | B |
| ATOM | 4223 | CE | LYS | 220 | 34.464 | 26.087 | 60.092 | 1.00 | 13.88 | B |
| ATOM | 4224 | NZ | LYS | 220 | 32.993 | 26.287 | 59.939 | 1.00 | 12.51 | B |
| ATOM | 4225 | C | LYS | 220 | 39.303 | 23.734 | 59.173 | 1.00 | 14.26 | B |
| ATOM | 4226 | O | LYS | 220 | 39.442 | 24.067 | 60.350 | 1.00 | 15.25 | B |
| ATOM | 4227 | N | ARG | 221 | 39.513 | 22.498 | 58.748 | 1.00 | 14.19 | B |
| ATOM | 4228 | CA | ARG | 221 | 39.936 | 21.438 | 59.647 | 1.00 | 11.64 | B |
| ATOM | 4229 | CB | ARG | 221 | 39.878 | 20.111 | 58.889 | 1.00 | 13.12 | B |
| ATOM | 4230 | CG | ARG | 221 | 40.038 | 18.857 | 59.751 | 1.00 | 13.06 | B |
| ATOM | 4231 | CD | ARG | 221 | 39.999 | 17.586 | 58.902 | 1.00 | 11.48 | B |
| ATOM | 4232 | NE | ARG | 221 | 38.638 | 17.093 | 58.691 | 1.00 | 8.87 | B |
| ATOM | 4233 | CZ | ARG | 221 | 38.317 | 16.184 | 57.774 | 1.00 | 8.38 | B |
| ATOM | 4234 | NH1 | ARG | 221 | 39.255 | 15.687 | 56.976 | 1.00 | 5.16 | B |
| ATOM | 4235 | NH2 | ARG | 221 | 37.074 | 15.732 | 57.687 | 1.00 | 8.15 | B |
| ATOM | 4236 | C | ARG | 221 | 41.345 | 21.737 | 60.174 | 1.00 | 10.67 | B |
| ATOM | 4237 | O | ARG | 221 | 41.686 | 21.394 | 61.314 | 1.00 | 10.15 | B |
| ATOM | 4238 | N | THR | 222 | 42.167 | 22.372 | 59.342 | 1.00 | 10.52 | B |
| ATOM | 4239 | CA | THR | 222 | 43.515 | 22.747 | 59.752 | 1.00 | 7.37 | B |
| ATOM | 4240 | CB | THR | 222 | 44.277 | 23.438 | 58.634 | 1.00 | 6.75 | B |
| ATOM | 4241 | OG1 | THR | 222 | 44.586 | 22.466 | 57.637 | 1.00 | 9.09 | B |
| ATOM | 4242 | CG2 | THR | 222 | 45.573 | 24.026 | 59.136 | 1.00 | 5.92 | B |
| ATOM | 4243 | C | THR | 222 | 43.475 | 23.692 | 60.916 | 1.00 | 5.52 | B |
| ATOM | 4244 | O | THR | 222 | 44.265 | 23.598 | 61.797 | 1.00 | 6.41 | B |
| ATOM | 4245 | N | THR | 223 | 42.527 | 24.607 | 60.906 | 1.00 | 5.73 | B |
| ATOM | 4246 | CA | THR | 223 | 42.443 | 25.550 | 61.990 | 1.00 | 7.41 | B |
| ATOM | 4247 | CB | THR | 223 | 41.481 | 26.706 | 61.654 | 1.00 | 9.80 | B |
| ATOM | 4248 | OG1 | THR | 223 | 40.126 | 26.260 | 61.807 | 1.00 | 13.96 | B |
| ATOM | 4249 | CG2 | THR | 223 | 41.716 | 27.205 | 60.212 | 1.00 | 11.03 | B |
| ATOM | 4250 | C | THR | 223 | 41.941 | 24.801 | 63.206 | 1.00 | 8.79 | B |
| ATOM | 4251 | O | THR | 223 | 42.353 | 25.101 | 64.337 | 1.00 | 11.00 | B |
| ATOM | 4252 | N | ALA | 224 | 41.093 | 23.796 | 62.970 | 1.00 | 9.46 | B |
| ATOM | 4253 | CA | ALA | 224 | 40.537 | 23.001 | 64.069 | 1.00 | 9.41 | B |
| ATOM | 4254 | CB | ALA | 224 | 39.514 | 21.966 | 63.570 | 1.00 | 8.72 | B |
| ATOM | 4255 | C | ALA | 224 | 41.645 | 22.288 | 64.798 | 1.00 | 10.87 | B |
| ATOM | 4256 | O | ALA | 224 | 41.693 | 22.258 | 66.041 | 1.00 | 10.92 | B |
| ATOM | 4257 | N | ALA | 225 | 42.526 | 21.678 | 64.020 | 1.00 | 11.03 | B |
| ATOM | 4258 | CA | ALA | 225 | 43.647 | 20.977 | 64.608 | 1.00 | 10.24 | B |
| ATOM | 4259 | CB | ALA | 225 | 44.484 | 20.347 | 63.517 | 1.00 | 9.24 | B |
| ATOM | 4260 | C | ALA | 225 | 44.502 | 21.942 | 65.446 | 1.00 | 11.63 | B |
| ATOM | 4261 | O | ALA | 225 | 44.983 | 21.592 | 66.516 | 1.00 | 12.58 | B |
| ATOM | 4262 | N | THR | 226 | 44.676 | 23.164 | 64.957 | 1.00 | 13.45 | B |
| ATOM | 4263 | CA | THR | 226 | 45.490 | 24.156 | 65.650 | 1.00 | 15.18 | B |
| ATOM | 4264 | CB | THR | 226 | 45.557 | 25.470 | 64.868 | 1.00 | 14.69 | B |
| ATOM | 4265 | OG1 | THR | 226 | 46.323 | 25.286 | 63.670 | 1.00 | 16.29 | B |
| ATOM | 4266 | CG2 | THR | 226 | 46.186 | 26.534 | 65.716 | 1.00 | 15.17 | B |
| ATOM | 4267 | C | THR | 226 | 44.901 | 24.452 | 67.007 | 1.00 | 16.64 | B |
| ATOM | 4268 | O | THR | 226 | 45.617 | 24.553 | 67.998 | 1.00 | 16.41 | B |
| ATOM | 4269 | N | LEU | 227 | 43.575 | 24.575 | 67.025 | 1.00 | 18.18 | B |
| ATOM | 4270 | CA | LEU | 227 | 42.805 | 24.875 | 68.238 | 1.00 | 18.74 | B |
| ATOM | 4271 | CB | LEU | 227 | 41.367 | 25.310 | 67.899 | 1.00 | 19.87 | B |
| ATOM | 4272 | CG | LEU | 227 | 40.955 | 26.772 | 68.051 | 1.00 | 21.86 | B |
| ATOM | 4273 | CD1 | LEU | 227 | 41.103 | 27.134 | 69.518 | 1.00 | 21.93 | B |
| ATOM | 4274 | CD2 | LEU | 227 | 41.786 | 27.693 | 67.155 | 1.00 | 21.51 | B |
| ATOM | 4275 | C | LEU | 227 | 42.651 | 23.733 | 69.239 | 1.00 | 18.17 | B |
| ATOM | 4276 | O | LEU | 227 | 42.783 | 23.928 | 70.435 | 1.00 | 18.61 | B |
| ATOM | 4277 | N | MET | 228 | 42.380 | 22.536 | 68.742 | 1.00 | 18.27 | B |
| ATOM | 4278 | CA | MET | 228 | 42.160 | 21.404 | 69.634 | 1.00 | 17.51 | B |
| ATOM | 4279 | CB | MET | 228 | 40.800 | 20.772 | 69.302 | 1.00 | 16.30 | B |
| ATOM | 4280 | CG | MET | 228 | 39.649 | 21.745 | 69.495 | 1.00 | 16.20 | B |
| ATOM | 4281 | SD | MET | 228 | 38.056 | 21.201 | 68.874 | 1.00 | 19.18 | B |
| ATOM | 4282 | CE | MET | 228 | 38.092 | 22.153 | 67.250 | 1.00 | 17.21 | B |
| ATOM | 4283 | C | MET | 228 | 43.250 | 20.342 | 69.614 | 1.00 | 18.14 | B |
| ATOM | 4284 | O | MET | 228 | 43.769 | 19.990 | 68.549 | 1.00 | 20.11 | B |
| ATOM | 4285 | N | ASN | 229 | 43.571 | 19.834 | 70.807 | 1.00 | 16.66 | B |
| ATOM | 4286 | CA | ASN | 229 | 44.589 | 18.799 | 70.992 | 1.00 | 16.35 | B |
| ATOM | 4287 | CB | ASN | 229 | 44.824 | 18.543 | 72.485 | 1.00 | 15.94 | B |
| ATOM | 4288 | CG | ASN | 229 | 45.350 | 19.764 | 73.209 | 1.00 | 16.33 | B |
| ATOM | 4289 | OD1 | ASN | 229 | 45.764 | 20.739 | 72.588 | 1.00 | 17.78 | B |
| ATOM | 4290 | ND2 | ASN | 229 | 45.340 | 19.711 | 74.534 | 1.00 | 14.68 | B |
| ATOM | 4291 | C | ASN | 229 | 44.311 | 17.448 | 70.313 | 1.00 | 15.68 | B |
| ATOM | 4292 | O | ASN | 229 | 43.228 | 16.873 | 70.460 | 1.00 | 15.38 | B |
| ATOM | 4293 | N | ALA | 230 | 45.300 | 16.950 | 69.569 | 1.00 | 14.15 | B |
| ATOM | 4294 | CA | ALA | 230 | 45.171 | 15.679 | 68.863 | 1.00 | 12.00 | B |
| ATOM | 4295 | CB | ALA | 230 | 45.241 | 14.546 | 69.847 | 1.00 | 11.64 | B |
| ATOM | 4296 | C | ALA | 230 | 43.869 | 15.595 | 68.079 | 1.00 | 11.58 | B |
| ATOM | 4297 | O | ALA | 230 | 43.269 | 14.519 | 67.977 | 1.00 | 10.16 | B |
| ATOM | 4298 | N | TYR | 231 | 43.443 | 16.725 | 67.519 | 1.00 | 11.27 | B |
| ATOM | 4299 | CA | TYR | 231 | 42.200 | 16.775 | 66.761 | 1.00 | 12.69 | B |
| ATOM | 4300 | CB | TYR | 231 | 42.047 | 18.119 | 66.029 | 1.00 | 11.10 | B |
| ATOM | 4301 | CG | TYR | 231 | 40.667 | 18.312 | 65.435 | 1.00 | 10.24 | B |
| ATOM | 4302 | CD1 | TYR | 231 | 40.404 | 17.998 | 64.112 | 1.00 | 9.88 | B |
| ATOM | 4303 | CE1 | TYR | 231 | 39.121 | 18.122 | 63.598 | 1.00 | 10.11 | B |
| ATOM | 4304 | CD2 | TYR | 231 | 39.606 | 18.760 | 66.229 | 1.00 | 11.37 | B |
| ATOM | 4305 | CE2 | TYR | 231 | 38.316 | 18.886 | 65.716 | 1.00 | 10.13 | B |
| ATOM | 4306 | CZ | TYR | 231 | 38.079 | 18.559 | 64.402 | 1.00 | 9.90 | B |
| ATOM | 4307 | OH | TYR | 231 | 36.780 | 18.623 | 63.936 | 1.00 | 7.41 | B |
| ATOM | 4308 | C | TYR | 231 | 41.988 | 15.645 | 65.748 | 1.00 | 13.47 | B |
| ATOM | 4309 | O | TYR | 231 | 41.016 | 14.916 | 65.837 | 1.00 | 14.47 | B |
| ATOM | 4310 | N | SER | 232 | 42.904 | 15.481 | 64.800 | 1.00 | 15.55 | B |
| ATOM | 4311 | CA | SER | 232 | 42.744 | 14.446 | 63.777 | 1.00 | 15.70 | B |
| ATOM | 4312 | CB | SER | 232 | 43.907 | 14.490 | 62.779 | 1.00 | 17.08 | B |
| ATOM | 4313 | OG | SER | 232 | 45.145 | 14.290 | 63.419 | 1.00 | 20.92 | B |
| ATOM | 4314 | C | SER | 232 | 42.608 | 13.020 | 64.308 | 1.00 | 15.28 | B |
| ATOM | 4315 | O | SER | 232 | 41.898 | 12.203 | 63.726 | 1.00 | 16.22 | B |
| ATOM | 4316 | N | SER | 233 | 43.260 | 12.711 | 65.417 | 1.00 | 12.45 | B |
| ATOM | 4317 | CA | SER | 233 | 43.173 | 11.352 | 65.919 | 1.00 | 12.60 | B |
| ATOM | 4318 | CB | SER | 233 | 44.477 | 10.942 | 66.596 | 1.00 | 13.54 | B |
| ATOM | 4319 | OG | SER | 233 | 44.662 | 11.602 | 67.838 | 1.00 | 15.82 | B |
| ATOM | 4320 | C | SER | 233 | 42.057 | 11.167 | 66.921 | 1.00 | 12.47 | B |
| ATOM | 4321 | O | SER | 233 | 41.604 | 10.047 | 67.155 | 1.00 | 12.18 | B |
| ATOM | 4322 | N | ARG | 234 | 41.612 | 12.265 | 67.523 | 1.00 | 11.28 | B |
| ATOM | 4323 | CA | ARG | 234 | 40.558 | 12.168 | 68.532 | 1.00 | 9.69 | B |
| ATOM | 4324 | CB | ARG | 234 | 40.919 | 12.961 | 69.784 | 1.00 | 10.96 | B |
| ATOM | 4325 | CG | ARG | 234 | 41.315 | 12.112 | 70.975 | 1.00 | 13.22 | B |
| ATOM | 4326 | CD | ARG | 234 | 42.707 | 12.435 | 71.494 | 1.00 | 16.77 | B |
| ATOM | 4327 | NE | ARG | 234 | 42.755 | 13.676 | 72.263 | 1.00 | 20.42 | B |
| ATOM | 4328 | CZ | ARG | 234 | 43.751 | 14.005 | 73.083 | 1.00 | 22.86 | B |
| ATOM | 4329 | NH1 | ARG | 234 | 44.791 | 13.186 | 73.242 | 1.00 | 22.37 | B |
| ATOM | 4330 | NH2 | ARG | 234 | 43.690 | 15.140 | 73.767 | 1.00 | 25.64 | B |
| ATOM | 4331 | C | ARG | 234 | 39.168 | 12.617 | 68.118 | 1.00 | 7.73 | B |
| ATOM | 4332 | O | ARG | 234 | 38.258 | 12.599 | 68.924 | 1.00 | 8.22 | B |
| ATOM | 4333 | N | SER | 235 | 39.006 | 13.014 | 66.862 | 1.00 | 6.52 | B |
| ATOM | 4334 | CA | SER | 235 | 37.697 | 13.455 | 66.394 | 1.00 | 4.31 | B |
| ATOM | 4335 | CB | SER | 235 | 37.785 | 14.801 | 65.647 | 1.00 | 2.24 | B |
| ATOM | 4336 | OG | SER | 235 | 38.745 | 14.780 | 64.602 | 1.00 | 1.00 | B |
| ATOM | 4337 | C | SER | 235 | 37.048 | 12.437 | 65.488 | 1.00 | 2.58 | B |
| ATOM | 4338 | O | SER | 235 | 37.704 | 11.648 | 64.854 | 1.00 | 3.58 | B |
| ATOM | 4339 | N | HIS | 236 | 35.725 | 12.465 | 65.472 | 1.00 | 4.87 | B |
| ATOM | 4340 | CA | HIS | 236 | 34.911 | 11.587 | 64.631 | 1.00 | 5.05 | B |
| ATOM | 4341 | CB | HIS | 236 | 33.691 | 11.087 | 65.386 | 1.00 | 4.65 | B |
| ATOM | 4342 | CG | HIS | 236 | 34.032 | 10.280 | 66.586 | 1.00 | 4.01 | B |
| ATOM | 4343 | CD2 | HIS | 236 | 34.066 | 10.607 | 67.899 | 1.00 | 3.63 | B |
| ATOM | 4344 | ND1 | HIS | 236 | 34.437 | 8.965 | 66.504 | 1.00 | 3.84 | B |
| ATOM | 4345 | CE1 | HIS | 236 | 34.704 | 8.517 | 67.717 | 1.00 | 4.48 | B |
| ATOM | 4346 | NE2 | HIS | 236 | 34.487 | 9.494 | 68.582 | 1.00 | 4.72 | B |
| ATOM | 4347 | C | HIS | 236 | 34.347 | 12.498 | 63.556 | 1.00 | 6.99 | B |
| ATOM | 4348 | O | HIS | 236 | 33.810 | 13.556 | 63.878 | 1.00 | 9.70 | B |
| ATOM | 4349 | N | SER | 237 | 34.475 | 12.108 | 62.291 | 1.00 | 7.23 | B |
| ATOM | 4350 | CA | SER | 237 | 33.951 | 12.933 | 61.208 | 1.00 | 6.69 | B |
| ATOM | 4351 | CB | SER | 237 | 35.058 | 13.406 | 60.253 | 1.00 | 5.37 | B |
| ATOM | 4352 | OG | SER | 237 | 35.464 | 12.358 | 59.380 | 1.00 | 3.60 | B |
| ATOM | 4353 | C | SER | 237 | 32.946 | 12.157 | 60.393 | 1.00 | 7.89 | B |
| ATOM | 4354 | O | SER | 237 | 33.196 | 11.040 | 59.976 | 1.00 | 9.95 | B |
| ATOM | 4355 | N | VAL | 238 | 31.787 | 12.753 | 60.180 | 1.00 | 7.91 | B |
| ATOM | 4356 | CA | VAL | 238 | 30.787 | 12.078 | 59.392 | 1.00 | 7.74 | B |
| ATOM | 4357 | CB | VAL | 238 | 29.560 | 11.740 | 60.282 | 1.00 | 8.04 | B |
| ATOM | 4358 | CG1 | VAL | 238 | 29.413 | 12.787 | 61.328 | 1.00 | 7.80 | B |
| ATOM | 4359 | CG2 | VAL | 238 | 28.307 | 11.669 | 59.460 | 1.00 | 8.71 | B |
| ATOM | 4360 | C | VAL | 238 | 30.421 | 12.935 | 58.182 | 1.00 | 8.25 | B |
| ATOM | 4361 | O | VAL | 238 | 29.776 | 13.952 | 58.323 | 1.00 | 9.09 | B |
| ATOM | 4362 | N | PHE | 239 | 30.883 | 12.511 | 57.002 | 1.00 | 8.31 | B |
| ATOM | 4363 | CA | PHE | 239 | 30.609 | 13.198 | 55.732 | 1.00 | 8.81 | B |
| ATOM | 4364 | CB | PHE | 239 | 31.793 | 13.036 | 54.759 | 1.00 | 6.73 | B |
| ATOM | 4365 | CG | PHE | 239 | 31.693 | 13.893 | 53.525 | 1.00 | 6.12 | B |
| ATOM | 4366 | CD1 | PHE | 239 | 30.815 | 13.557 | 52.500 | 1.00 | 5.69 | B |
| ATOM | 4367 | CD2 | PHE | 239 | 32.462 | 15.046 | 53.394 | 1.00 | 5.95 | B |
| ATOM | 4368 | CE1 | PHE | 239 | 30.705 | 14.364 | 51.348 | 1.00 | 5.30 | B |
| ATOM | 4369 | CE2 | PHE | 239 | 32.354 | 15.854 | 52.247 | 1.00 | 5.11 | B |
| ATOM | 4370 | CZ | PHE | 239 | 31.475 | 15.511 | 51.224 | 1.00 | 3.58 | B |
| ATOM | 4371 | C | PHE | 239 | 29.350 | 12.553 | 55.148 | 1.00 | 9.90 | B |
| ATOM | 4372 | O | PHE | 239 | 29.327 | 11.356 | 54.859 | 1.00 | 9.81 | B |
| ATOM | 4373 | N | SER | 240 | 28.305 | 13.359 | 54.982 | 1.00 | 10.63 | B |
| ATOM | 4374 | CA | SER | 240 | 27.039 | 12.871 | 54.466 | 1.00 | 9.05 | B |
| ATOM | 4375 | CB | SER | 240 | 25.926 | 13.194 | 55.467 | 1.00 | 9.24 | B |
| ATOM | 4376 | OG | SER | 240 | 26.182 | 12.631 | 56.742 | 1.00 | 8.98 | B |
| ATOM | 4377 | C | SER | 240 | 26.678 | 13.462 | 53.105 | 1.00 | 10.23 | B |
| ATOM | 4378 | O | SER | 240 | 26.809 | 14.668 | 52.877 | 1.00 | 10.82 | B |
| ATOM | 4379 | N | VAL | 241 | 26.230 | 12.601 | 52.198 | 1.00 | 10.77 | B |
| ATOM | 4380 | CA | VAL | 241 | 25.813 | 13.044 | 50.874 | 1.00 | 12.14 | B |
| ATOM | 4381 | CB | VAL | 241 | 26.748 | 12.492 | 49.775 | 1.00 | 12.12 | B |
| ATOM | 4382 | CG1 | VAL | 241 | 26.981 | 11.008 | 50.002 | 1.00 | 13.27 | B |
| ATOM | 4383 | CG2 | VAL | 241 | 26.143 | 12.736 | 48.394 | 1.00 | 11.17 | B |
| ATOM | 4384 | C | VAL | 241 | 24.379 | 12.565 | 50.649 | 1.00 | 13.61 | B |
| ATOM | 4385 | O | VAL | 241 | 24.092 | 11.365 | 50.700 | 1.00 | 13.01 | B |
| ATOM | 4386 | N | THR | 242 | 23.478 | 13.513 | 50.422 | 1.00 | 14.36 | B |
| ATOM | 4387 | CA | THR | 242 | 22.078 | 13.203 | 50.217 | 1.00 | 16.18 | B |
| ATOM | 4388 | CB | THR | 242 | 21.198 | 14.104 | 51.118 | 1.00 | 17.52 | B |
| ATOM | 4389 | OG1 | THR | 242 | 21.546 | 13.897 | 52.496 | 1.00 | 19.73 | B |
| ATOM | 4390 | CG2 | THR | 242 | 19.738 | 13.766 | 50.954 | 1.00 | 20.46 | B |
| ATOM | 4391 | C | THR | 242 | 21.746 | 13.418 | 48.741 | 1.00 | 18.15 | B |
| ATOM | 4392 | O | THR | 242 | 22.212 | 14.357 | 48.128 | 1.00 | 19.20 | B |
| ATOM | 4393 | N | ILE | 243 | 20.945 | 12.521 | 48.180 | 1.00 | 20.44 | B |
| ATOM | 4394 | CA | ILE | 243 | 20.560 | 12.619 | 46.785 | 1.00 | 23.13 | B |
| ATOM | 4395 | CB | ILE | 243 | 21.178 | 11.477 | 45.941 | 1.00 | 22.27 | B |
| ATOM | 4396 | CG2 | ILE | 243 | 20.962 | 11.770 | 44.475 | 1.00 | 18.06 | B |
| ATOM | 4397 | CG1 | ILE | 243 | 22.663 | 11.310 | 46.270 | 1.00 | 21.29 | B |
| ATOM | 4398 | CD1 | ILE | 243 | 23.247 | 10.072 | 45.722 | 1.00 | 21.09 | B |
| ATOM | 4399 | C | ILE | 243 | 19.043 | 12.555 | 46.628 | 1.00 | 26.42 | B |
| ATOM | 4400 | O | ILE | 243 | 18.442 | 11.488 | 46.790 | 1.00 | 27.92 | B |
| ATOM | 4401 | N | HIS | 244 | 18.437 | 13.707 | 46.340 | 1.00 | 29.29 | B |
| ATOM | 4402 | CA | HIS | 244 | 17.001 | 13.808 | 46.117 | 1.00 | 30.50 | B |
| ATOM | 4403 | CB | HIS | 244 | 16.486 | 15.226 | 46.393 | 1.00 | 31.87 | B |
| ATOM | 4404 | CG | HIS | 244 | 16.375 | 15.565 | 47.845 | 1.00 | 34.67 | B |
| ATOM | 4405 | CD2 | HIS | 244 | 15.341 | 15.441 | 48.712 | 1.00 | 35.28 | B |
| ATOM | 4406 | ND1 | HIS | 244 | 17.424 | 16.087 | 48.577 | 1.00 | 36.67 | B |
| ATOM | 4407 | CE1 | HIS | 244 | 17.040 | 16.267 | 49.828 | 1.00 | 35.69 | B |
| ATOM | 4408 | NE2 | HIS | 244 | 15.778 | 15.881 | 49.936 | 1.00 | 35.59 | B |
| ATOM | 4409 | C | HIS | 244 | 16.803 | 13.494 | 44.637 | 1.00 | 32.12 | B |
| ATOM | 4410 | O | HIS | 244 | 17.277 | 14.228 | 43.755 | 1.00 | 32.44 | B |
| ATOM | 4411 | N | MET | 245 | 16.122 | 12.388 | 44.368 | 1.00 | 32.37 | B |
| ATOM | 4412 | CA | MET | 245 | 15.877 | 11.968 | 42.998 | 1.00 | 32.37 | B |
| ATOM | 4413 | CB | MET | 245 | 16.475 | 10.578 | 42.791 | 1.00 | 31.86 | B |
| ATOM | 4414 | CG | MET | 245 | 17.968 | 10.548 | 43.055 | 1.00 | 31.73 | B |
| ATOM | 4415 | SD | MET | 245 | 18.589 | 8.875 | 43.225 | 1.00 | 33.02 | B |
| ATOM | 4416 | CE | MET | 245 | 18.034 | 8.477 | 44.892 | 1.00 | 31.10 | B |
| ATOM | 4417 | C | MET | 245 | 14.401 | 12.002 | 42.601 | 1.00 | 31.83 | B |
| ATOM | 4418 | O | MET | 245 | 13.509 | 11.738 | 43.415 | 1.00 | 31.92 | B |
| ATOM | 4419 | N | LYS | 246 | 14.159 | 12.334 | 41.337 | 1.00 | 31.84 | B |
| ATOM | 4420 | CA | LYS | 246 | 12.811 | 12.428 | 40.804 | 1.00 | 31.99 | B |
| ATOM | 4421 | CB | LYS | 246 | 12.350 | 13.895 | 40.781 | 1.00 | 32.10 | B |
| ATOM | 4422 | CG | LYS | 246 | 10.922 | 14.087 | 40.292 | 1.00 | 34.26 | B |
| ATOM | 4423 | CD | LYS | 246 | 10.606 | 15.539 | 39.946 | 1.00 | 34.52 | B |
| ATOM | 4424 | CE | LYS | 246 | 10.646 | 16.433 | 41.173 | 1.00 | 36.15 | B |
| ATOM | 4425 | NZ | LYS | 246 | 10.457 | 17.872 | 40.836 | 1.00 | 35.42 | B |
| ATOM | 4426 | C | LYS | 246 | 12.761 | 11.870 | 39.382 | 1.00 | 31.58 | B |
| ATOM | 4427 | O | LYS | 246 | 13.439 | 12.358 | 38.480 | 1.00 | 30.24 | B |
| ATOM | 4428 | N | GLU | 247 | 11.967 | 10.824 | 39.196 | 1.00 | 31.71 | B |
| ATOM | 4429 | CA | GLU | 247 | 11.808 | 10.238 | 37.874 | 1.00 | 30.99 | B |
| ATOM | 4430 | CB | GLU | 247 | 12.337 | 8.801 | 37.855 | 1.00 | 32.21 | B |
| ATOM | 4431 | CG | GLU | 247 | 11.815 | 7.897 | 38.961 | 1.00 | 33.61 | B |
| ATOM | 4432 | CD | GLU | 247 | 12.672 | 6.647 | 39.115 | 1.00 | 35.27 | B |
| ATOM | 4433 | OE1 | GLU | 247 | 12.420 | 5.841 | 40.037 | 1.00 | 35.63 | B |
| ATOM | 4434 | OE2 | GLU | 247 | 13.609 | 6.469 | 38.307 | 1.00 | 35.39 | B |
| ATOM | 4435 | C | GLU | 247 | 10.338 | 10.298 | 37.479 | 1.00 | 30.04 | B |
| ATOM | 4436 | O | GLU | 247 | 9.448 | 10.169 | 38.317 | 1.00 | 29.68 | B |
| ATOM | 4437 | N | THR | 248 | 10.083 | 10.513 | 36.197 | 1.00 | 28.13 | B |
| ATOM | 4438 | CA | THR | 248 | 8.716 | 10.591 | 35.720 | 1.00 | 26.83 | B |
| ATOM | 4439 | CB | THR | 248 | 8.506 | 11.895 | 34.942 | 1.00 | 25.80 | B |
| ATOM | 4440 | OG1 | THR | 248 | 8.937 | 12.995 | 35.750 | 1.00 | 24.67 | B |
| ATOM | 4441 | CG2 | THR | 248 | 7.046 | 12.096 | 34.617 | 1.00 | 25.62 | B |
| ATOM | 4442 | C | THR | 248 | 8.406 | 9.395 | 34.822 | 1.00 | 26.77 | B |
| ATOM | 4443 | O | THR | 248 | 9.168 | 9.077 | 33.914 | 1.00 | 27.38 | B |
| ATOM | 4444 | N | THR | 249 | 7.288 | 8.732 | 35.092 | 1.00 | 26.76 | B |
| ATOM | 4445 | CA | THR | 249 | 6.877 | 7.580 | 34.302 | 1.00 | 26.72 | B |
| ATOM | 4446 | CB | THR | 249 | 5.759 | 6.784 | 35.011 | 1.00 | 26.45 | B |
| ATOM | 4447 | OG1 | THR | 249 | 4.575 | 7.587 | 35.088 | 1.00 | 27.92 | B |
| ATOM | 4448 | CG2 | THR | 249 | 6.180 | 6.404 | 36.423 | 1.00 | 25.26 | B |
| ATOM | 4449 | C | THR | 249 | 6.353 | 8.040 | 32.938 | 1.00 | 27.55 | B |
| ATOM | 4450 | O | THR | 249 | 6.316 | 9.226 | 32.638 | 1.00 | 27.26 | B |
| ATOM | 4451 | N | ILE | 250 | 5.956 | 7.078 | 32.113 | 1.00 | 29.51 | B |
| ATOM | 4452 | CA | ILE | 250 | 5.434 | 7.353 | 30.774 | 1.00 | 30.16 | B |
| ATOM | 4453 | CB | ILE | 250 | 5.444 | 6.074 | 29.901 | 1.00 | 29.03 | B |
| ATOM | 4454 | CG2 | ILE | 250 | 4.410 | 5.082 | 30.421 | 1.00 | 27.86 | B |
| ATOM | 4455 | CG1 | ILE | 250 | 5.157 | 6.431 | 28.443 | 1.00 | 28.33 | B |
| ATOM | 4456 | CD1 | ILE | 250 | 5.425 | 5.295 | 27.476 | 1.00 | 26.91 | B |
| ATOM | 4457 | C | ILE | 250 | 4.005 | 7.884 | 30.877 | 1.00 | 31.97 | B |
| ATOM | 4458 | O | ILE | 250 | 3.400 | 8.286 | 29.891 | 1.00 | 31.50 | B |
| ATOM | 4459 | N | ASP | 251 | 3.477 | 7.875 | 32.095 | 1.00 | 34.02 | B |
| ATOM | 4460 | CA | ASP | 251 | 2.132 | 8.368 | 32.359 | 1.00 | 36.26 | B |
| ATOM | 4461 | CB | ASP | 251 | 1.425 | 7.469 | 33.381 | 1.00 | 36.12 | B |
| ATOM | 4462 | CG | ASP | 251 | 0.789 | 6.242 | 32.750 | 1.00 | 36.40 | B |
| ATOM | 4463 | OD1 | ASP | 251 | 0.223 | 5.420 | 33.509 | 1.00 | 34.19 | B |
| ATOM | 4464 | OD2 | ASP | 251 | 0.854 | 6.119 | 31.504 | 1.00 | 36.03 | B |
| ATOM | 4465 | C | ASP | 251 | 2.164 | 9.804 | 32.910 | 1.00 | 37.47 | B |
| ATOM | 4466 | O | ASP | 251 | 1.140 | 10.468 | 32.990 | 1.00 | 38.11 | B |
| ATOM | 4467 | N | GLY | 252 | 3.350 | 10.273 | 33.284 | 1.00 | 37.77 | B |
| ATOM | 4468 | CA | GLY | 252 | 3.471 | 11.613 | 33.822 | 1.00 | 37.41 | B |
| ATOM | 4469 | C | GLY | 252 | 3.566 | 11.662 | 35.338 | 1.00 | 38.71 | B |
| ATOM | 4470 | O | GLY | 252 | 3.747 | 12.734 | 35.912 | 1.00 | 38.78 | B |
| ATOM | 4471 | N | GLU | 253 | 3.440 | 10.516 | 36.003 | 1.00 | 38.85 | B |
| ATOM | 4472 | CA | GLU | 253 | 3.533 | 10.511 | 37.459 | 1.00 | 39.67 | B |
| ATOM | 4473 | CB | GLU | 253 | 3.020 | 9.200 | 38.052 | 1.00 | 41.37 | B |
| ATOM | 4474 | CG | GLU | 253 | 3.181 | 9.143 | 39.573 | 1.00 | 43.75 | B |
| ATOM | 4475 | CD | GLU | 253 | 2.814 | 7.803 | 40.188 | 1.00 | 44.31 | B |
| ATOM | 4476 | OE1 | GLU | 253 | 3.083 | 7.612 | 41.398 | 1.00 | 44.42 | B |
| ATOM | 4477 | OE2 | GLU | 253 | 2.256 | 6.945 | 39.470 | 1.00 | 45.10 | B |
| ATOM | 4478 | C | GLU | 253 | 4.988 | 10.668 | 37.883 | 1.00 | 39.49 | B |
| ATOM | 4479 | O | GLU | 253 | 5.890 | 10.286 | 37.149 | 1.00 | 39.20 | B |
| ATOM | 4480 | N | GLU | 254 | 5.210 | 11.239 | 39.064 | 1.00 | 39.27 | B |
| ATOM | 4481 | CA | GLU | 254 | 6.568 | 11.426 | 39.567 | 1.00 | 40.50 | B |
| ATOM | 4482 | CB | GLU | 254 | 6.793 | 12.875 | 39.978 | 1.00 | 41.13 | B |
| ATOM | 4483 | CG | GLU | 254 | 6.621 | 13.842 | 38.836 | 1.00 | 44.09 | B |
| ATOM | 4484 | CD | GLU | 254 | 7.073 | 15.233 | 39.189 | 1.00 | 45.25 | B |
| ATOM | 4485 | OE1 | GLU | 254 | 6.665 | 15.737 | 40.256 | 1.00 | 45.35 | B |
| ATOM | 4486 | OE2 | GLU | 254 | 7.828 | 15.825 | 38.391 | 1.00 | 46.38 | B |
| ATOM | 4487 | C | GLU | 254 | 6.926 | 10.539 | 40.756 | 1.00 | 39.50 | B |
| ATOM | 4488 | O | GLU | 254 | 6.242 | 10.540 | 41.769 | 1.00 | 40.75 | B |
| ATOM | 4489 | N | LEU | 255 | 8.008 | 9.779 | 40.614 | 1.00 | 37.82 | B |
| ATOM | 4490 | CA | LEU | 255 | 8.484 | 8.894 | 41.676 | 1.00 | 36.11 | B |
| ATOM | 4491 | CB | LEU | 255 | 8.895 | 7.543 | 41.087 | 1.00 | 35.93 | B |
| ATOM | 4492 | CG | LEU | 255 | 7.950 | 6.910 | 40.062 | 1.00 | 35.67 | B |
| ATOM | 4493 | CD1 | LEU | 255 | 8.538 | 5.614 | 39.590 | 1.00 | 35.24 | B |
| ATOM | 4494 | CD2 | LEU | 255 | 6.601 | 6.663 | 40.668 | 1.00 | 35.26 | B |
| ATOM | 4495 | C | LEU | 255 | 9.710 | 9.551 | 42.322 | 1.00 | 35.19 | B |
| ATOM | 4496 | O | LEU | 255 | 10.722 | 9.754 | 41.644 | 1.00 | 35.09 | B |
| ATOM | 4497 | N | VAL | 256 | 9.612 | 9.888 | 43.615 | 1.00 | 33.29 | B |
| ATOM | 4498 | CA | VAL | 256 | 10.719 | 10.528 | 44.350 | 1.00 | 31.53 | B |
| ATOM | 4499 | CB | VAL | 256 | 10.237 | 11.748 | 45.143 | 1.00 | 31.44 | B |
| ATOM | 4500 | CG1 | VAL | 256 | 9.719 | 12.800 | 44.188 | 1.00 | 30.73 | B |
| ATOM | 4501 | CG2 | VAL | 256 | 9.165 | 11.322 | 46.141 | 1.00 | 33.02 | B |
| ATOM | 4502 | C | VAL | 256 | 11.494 | 9.622 | 45.319 | 1.00 | 29.50 | B |
| ATOM | 4503 | O | VAL | 256 | 10.928 | 8.958 | 46.189 | 1.00 | 29.05 | B |
| ATOM | 4504 | N | LYS | 257 | 12.809 | 9.604 | 45.148 | 1.00 | 27.07 | B |
| ATOM | 4505 | CA | LYS | 257 | 13.676 | 8.790 | 45.985 | 1.00 | 24.38 | B |
| ATOM | 4506 | CB | LYS | 257 | 14.530 | 7.832 | 45.134 | 1.00 | 21.73 | B |
| ATOM | 4507 | CG | LYS | 257 | 13.742 | 6.776 | 44.369 | 1.00 | 18.70 | B |
| ATOM | 4508 | CD | LYS | 257 | 14.637 | 5.862 | 43.566 | 1.00 | 13.96 | B |
| ATOM | 4509 | CE | LYS | 257 | 15.316 | 6.632 | 42.460 | 1.00 | 12.43 | B |
| ATOM | 4510 | NZ | LYS | 257 | 16.093 | 5.743 | 41.576 | 1.00 | 10.28 | B |
| ATOM | 4511 | C | LYS | 257 | 14.627 | 9.701 | 46.731 | 1.00 | 23.77 | B |
| ATOM | 4512 | O | LYS | 257 | 15.062 | 10.708 | 46.215 | 1.00 | 24.31 | B |
| ATOM | 4513 | N | ILE | 258 | 14.928 | 9.357 | 47.970 | 1.00 | 22.97 | B |
| ATOM | 4514 | CA | ILE | 258 | 15.882 | 10.138 | 48.741 | 1.00 | 20.65 | B |
| ATOM | 4515 | CB | ILE | 258 | 15.226 | 10.866 | 49.913 | 1.00 | 22.22 | B |
| ATOM | 4516 | CG2 | ILE | 258 | 16.246 | 11.747 | 50.591 | 1.00 | 22.81 | B |
| ATOM | 4517 | CG1 | ILE | 258 | 14.080 | 11.734 | 49.407 | 1.00 | 24.53 | B |
| ATOM | 4518 | CD1 | ILE | 258 | 13.276 | 12.417 | 50.518 | 1.00 | 24.98 | B |
| ATOM | 4519 | C | ILE | 258 | 16.891 | 9.136 | 49.271 | 1.00 | 18.47 | B |
| ATOM | 4520 | O | ILE | 258 | 16.554 | 8.243 | 50.049 | 1.00 | 16.24 | B |
| ATOM | 4521 | N | GLY | 259 | 18.123 | 9.256 | 48.805 | 1.00 | 17.79 | B |
| ATOM | 4522 | CA | GLY | 259 | 19.144 | 8.342 | 49.262 | 1.00 | 18.70 | B |
| ATOM | 4523 | C | GLY | 259 | 20.205 | 9.094 | 50.030 | 1.00 | 17.80 | B |
| ATOM | 4524 | O | GLY | 259 | 20.684 | 10.110 | 49.555 | 1.00 | 18.70 | B |
| ATOM | 4525 | N | LYS | 260 | 20.565 | 8.606 | 51.215 | 1.00 | 16.12 | B |
| ATOM | 4526 | CA | LYS | 260 | 21.598 | 9.263 | 52.011 | 1.00 | 15.58 | B |
| ATOM | 4527 | CB | LYS | 260 | 21.034 | 9.800 | 53.335 | 1.00 | 15.55 | B |
| ATOM | 4528 | CG | LYS | 260 | 21.889 | 10.844 | 54.046 | 1.00 | 14.21 | B |
| ATOM | 4529 | CD | LYS | 260 | 21.173 | 11.288 | 55.341 | 1.00 | 15.40 | B |
| ATOM | 4530 | CE | LYS | 260 | 21.989 | 12.289 | 56.170 | 1.00 | 13.76 | B |
| ATOM | 4531 | NZ | LYS | 260 | 21.311 | 12.687 | 57.451 | 1.00 | 8.49 | B |
| ATOM | 4532 | C | LYS | 260 | 22.729 | 8.309 | 52.335 | 1.00 | 13.87 | B |
| ATOM | 4533 | O | LYS | 260 | 22.531 | 7.185 | 52.741 | 1.00 | 13.84 | B |
| ATOM | 4534 | N | LEU | 261 | 23.937 | 8.788 | 52.141 | 1.00 | 13.07 | B |
| ATOM | 4535 | CA | LEU | 261 | 25.107 | 7.996 | 52.430 | 1.00 | 11.82 | B |
| ATOM | 4536 | CB | LEU | 261 | 25.890 | 7.772 | 51.130 | 1.00 | 10.77 | B |
| ATOM | 4537 | CG | LEU | 261 | 27.276 | 7.138 | 51.238 | 1.00 | 8.89 | B |
| ATOM | 4538 | CD1 | LEU | 261 | 27.189 | 5.799 | 51.975 | 1.00 | 7.84 | B |
| ATOM | 4539 | CD2 | LEU | 261 | 27.847 | 6.973 | 49.840 | 1.00 | 7.48 | B |
| ATOM | 4540 | C | LEU | 261 | 25.993 | 8.696 | 53.465 | 1.00 | 11.44 | B |
| ATOM | 4541 | O | LEU | 261 | 26.424 | 9.819 | 53.247 | 1.00 | 13.74 | B |
| ATOM | 4542 | N | ASN | 262 | 26.245 | 8.024 | 54.586 | 1.00 | 10.57 | B |
| ATOM | 4543 | CA | ASN | 262 | 27.142 | 8.548 | 55.615 | 1.00 | 8.04 | B |
| ATOM | 4544 | CB | ASN | 262 | 26.494 | 8.386 | 56.985 | 1.00 | 5.60 | B |
| ATOM | 4545 | CG | ASN | 262 | 25.111 | 8.980 | 57.011 | 1.00 | 8.99 | B |
| ATOM | 4546 | OD1 | ASN | 262 | 24.100 | 8.263 | 56.971 | 1.00 | 9.21 | B |
| ATOM | 4547 | ND2 | ASN | 262 | 25.050 | 10.307 | 57.024 | 1.00 | 7.62 | B |
| ATOM | 4548 | C | ASN | 262 | 28.526 | 7.879 | 55.554 | 1.00 | 6.87 | B |
| ATOM | 4549 | O | ASN | 262 | 28.640 | 6.653 | 55.523 | 1.00 | 7.74 | B |
| ATOM | 4550 | N | LEU | 263 | 29.566 | 8.705 | 55.487 | 1.00 | 5.79 | B |
| ATOM | 4551 | CA | LEU | 263 | 30.938 | 8.225 | 55.438 | 1.00 | 5.65 | B |
| ATOM | 4552 | CB | LEU | 263 | 31.596 | 8.741 | 54.165 | 1.00 | 4.90 | B |
| ATOM | 4553 | CG | LEU | 263 | 30.735 | 8.279 | 52.998 | 1.00 | 6.08 | B |
| ATOM | 4554 | CD1 | LEU | 263 | 31.131 | 9.012 | 51.752 | 1.00 | 5.33 | B |
| ATOM | 4555 | CD2 | LEU | 263 | 30.853 | 6.748 | 52.877 | 1.00 | 6.04 | B |
| ATOM | 4556 | C | LEU | 263 | 31.634 | 8.694 | 56.710 | 1.00 | 6.26 | B |
| ATOM | 4557 | O | LEU | 263 | 32.017 | 9.853 | 56.842 | 1.00 | 8.01 | B |
| ATOM | 4558 | N | VAL | 264 | 31.795 | 7.778 | 57.653 | 1.00 | 6.21 | B |
| ATOM | 4559 | CA | VAL | 264 | 32.406 | 8.079 | 58.943 | 1.00 | 6.25 | B |
| ATOM | 4560 | CB | VAL | 264 | 31.600 | 7.410 | 60.037 | 1.00 | 7.30 | B |
| ATOM | 4561 | CG1 | VAL | 264 | 32.081 | 7.848 | 61.406 | 1.00 | 6.21 | B |
| ATOM | 4562 | CG2 | VAL | 264 | 30.140 | 7.709 | 59.802 | 1.00 | 9.51 | B |
| ATOM | 4563 | C | VAL | 264 | 33.863 | 7.677 | 59.150 | 1.00 | 7.28 | B |
| ATOM | 4564 | O | VAL | 264 | 34.221 | 6.532 | 58.978 | 1.00 | 7.31 | B |
| ATOM | 4565 | N | ASP | 265 | 34.685 | 8.652 | 59.533 | 1.00 | 9.79 | B |
| ATOM | 4566 | CA | ASP | 265 | 36.105 | 8.441 | 59.841 | 1.00 | 11.34 | B |
| ATOM | 4567 | CB | ASP | 265 | 36.978 | 9.564 | 59.262 | 1.00 | 12.62 | B |
| ATOM | 4568 | CG | ASP | 265 | 38.473 | 9.346 | 59.520 | 1.00 | 16.17 | B |
| ATOM | 4569 | OD1 | ASP | 265 | 38.801 | 8.748 | 60.562 | 1.00 | 17.08 | B |
| ATOM | 4570 | OD2 | ASP | 265 | 39.310 | 9.783 | 58.694 | 1.00 | 16.43 | B |
| ATOM | 4571 | C | ASP | 265 | 36.179 | 8.527 | 61.374 | 1.00 | 11.75 | B |
| ATOM | 4572 | O | ASP | 265 | 36.356 | 9.601 | 61.928 | 1.00 | 11.74 | B |
| ATOM | 4573 | N | LEU | 266 | 36.032 | 7.389 | 62.051 | 1.00 | 12.21 | B |
| ATOM | 4574 | CA | LEU | 266 | 36.054 | 7.367 | 63.519 | 1.00 | 13.54 | B |
| ATOM | 4575 | CB | LEU | 266 | 35.692 | 5.986 | 64.068 | 1.00 | 13.06 | B |
| ATOM | 4576 | CG | LEU | 266 | 34.327 | 5.426 | 63.711 | 1.00 | 14.69 | B |
| ATOM | 4577 | CD1 | LEU | 266 | 34.190 | 3.979 | 64.232 | 1.00 | 13.37 | B |
| ATOM | 4578 | CD2 | LEU | 266 | 33.266 | 6.350 | 64.285 | 1.00 | 14.29 | B |
| ATOM | 4579 | C | LEU | 266 | 37.366 | 7.763 | 64.193 | 1.00 | 14.66 | B |
| ATOM | 4580 | O | LEU | 266 | 38.437 | 7.776 | 63.580 | 1.00 | 16.77 | B |
| ATOM | 4581 | N | ALA | 267 | 37.267 | 8.097 | 65.474 | 1.00 | 15.57 | B |
| ATOM | 4582 | CA | ALA | 267 | 38.435 | 8.494 | 66.237 | 1.00 | 15.49 | B |
| ATOM | 4583 | CB | ALA | 267 | 38.015 | 9.063 | 67.584 | 1.00 | 15.66 | B |
| ATOM | 4584 | C | ALA | 267 | 39.281 | 7.256 | 66.427 | 1.00 | 16.90 | B |
| ATOM | 4585 | O | ALA | 267 | 38.752 | 6.166 | 66.492 | 1.00 | 17.09 | B |
| ATOM | 4586 | N | GLY | 268 | 40.594 | 7.432 | 66.535 | 1.00 | 18.45 | B |
| ATOM | 4587 | CA | GLY | 268 | 41.470 | 6.286 | 66.684 | 1.00 | 19.06 | B |
| ATOM | 4588 | C | GLY | 268 | 40.979 | 5.375 | 67.779 | 1.00 | 20.29 | B |
| ATOM | 4589 | O | GLY | 268 | 40.476 | 5.846 | 68.778 | 1.00 | 22.63 | B |
| ATOM | 4590 | N | SER | 269 | 41.153 | 4.070 | 67.608 | 1.00 | 21.30 | B |
| ATOM | 4591 | CA | SER | 269 | 40.683 | 3.127 | 68.611 | 1.00 | 21.55 | B |
| ATOM | 4592 | CB | SER | 269 | 40.151 | 1.869 | 67.940 | 1.00 | 19.85 | B |
| ATOM | 4593 | OG | SER | 269 | 41.174 | 1.230 | 67.206 | 1.00 | 19.77 | B |
| ATOM | 4594 | C | SER | 269 | 41.696 | 2.703 | 69.666 | 1.00 | 23.07 | B |
| ATOM | 4595 | O | SER | 269 | 41.415 | 1.832 | 70.461 | 1.00 | 23.77 | B |
| ATOM | 4596 | N | GLU | 270 | 42.863 | 3.336 | 69.682 | 1.00 | 24.72 | B |
| ATOM | 4597 | CA | GLU | 270 | 43.889 | 2.997 | 70.666 | 1.00 | 26.45 | B |
| ATOM | 4598 | CB | GLU | 270 | 45.255 | 3.538 | 70.212 | 1.00 | 26.88 | B |
| ATOM | 4599 | CG | GLU | 270 | 45.365 | 5.074 | 70.179 | 1.00 | 26.65 | B |
| ATOM | 4600 | CD | GLU | 270 | 44.769 | 5.716 | 68.938 | 1.00 | 25.63 | B |
| ATOM | 4601 | OE1 | GLU | 270 | 44.782 | 6.966 | 68.848 | 1.00 | 25.90 | B |
| ATOM | 4602 | OE2 | GLU | 270 | 44.299 | 4.966 | 68.063 | 1.00 | 25.37 | B |
| ATOM | 4603 | C | GLU | 270 | 43.595 | 3.501 | 72.096 | 1.00 | 28.21 | B |
| ATOM | 4604 | O | GLU | 270 | 43.182 | 4.646 | 72.317 | 1.00 | 27.82 | B |
| ATOM | 4605 | N | ASN | 271 | 43.804 | 2.619 | 73.066 | 1.00 | 31.11 | B |
| ATOM | 4606 | CA | ASN | 271 | 43.590 | 2.932 | 74.483 | 1.00 | 33.53 | B |
| ATOM | 4607 | CB | ASN | 271 | 42.239 | 3.620 | 74.720 | 1.00 | 35.28 | B |
| ATOM | 4608 | CG | ASN | 271 | 41.046 | 2.755 | 74.319 | 1.00 | 37.15 | B |
| ATOM | 4609 | OD1 | ASN | 271 | 39.892 | 3.159 | 74.481 | 1.00 | 37.89 | B |
| ATOM | 4610 | ND2 | ASN | 271 | 41.319 | 1.569 | 73.789 | 1.00 | 38.13 | B |
| ATOM | 4611 | C | ASN | 271 | 43.617 | 1.669 | 75.326 | 1.00 | 34.61 | B |
| ATOM | 4612 | O | ASN | 271 | 43.637 | 0.561 | 74.789 | 1.00 | 35.03 | B |
| ATOM | 4613 | N | ASN | 287 | 41.713 | 11.898 | 79.742 | 1.00 | 41.72 | B |
| ATOM | 4614 | CA | ASN | 287 | 40.726 | 12.291 | 78.737 | 1.00 | 42.10 | B |
| ATOM | 4615 | CB | ASN | 287 | 41.389 | 13.166 | 77.666 | 1.00 | 43.36 | B |
| ATOM | 4616 | CG | ASN | 287 | 42.137 | 14.334 | 78.263 | 1.00 | 44.01 | B |
| ATOM | 4617 | OD1 | ASN | 287 | 43.107 | 14.144 | 78.990 | 1.00 | 44.40 | B |
| ATOM | 4618 | ND2 | ASN | 287 | 41.688 | 15.548 | 77.967 | 1.00 | 44.56 | B |
| ATOM | 4619 | C | ASN | 287 | 40.094 | 11.054 | 78.083 | 1.00 | 41.01 | B |
| ATOM | 4620 | O | ASN | 287 | 40.802 | 10.130 | 77.661 | 1.00 | 42.34 | B |
| ATOM | 4621 | N | ILE | 288 | 38.764 | 11.039 | 77.994 | 1.00 | 37.53 | B |
| ATOM | 4622 | CA | ILE | 288 | 38.053 | 9.905 | 77.397 | 1.00 | 33.20 | B |
| ATOM | 4623 | CB | ILE | 288 | 37.119 | 9.256 | 78.433 | 1.00 | 33.55 | B |
| ATOM | 4624 | CG2 | ILE | 288 | 37.940 | 8.681 | 79.575 | 1.00 | 32.67 | B |
| ATOM | 4625 | CG1 | ILE | 288 | 36.142 | 10.308 | 78.967 | 1.00 | 33.79 | B |
| ATOM | 4626 | CD1 | ILE | 288 | 35.028 | 9.764 | 79.828 | 1.00 | 33.58 | B |
| ATOM | 4627 | C | ILE | 288 | 37.221 | 10.255 | 76.147 | 1.00 | 29.09 | B |
| ATOM | 4628 | O | ILE | 288 | 36.810 | 11.410 | 75.946 | 1.00 | 28.30 | B |
| ATOM | 4629 | N | ASN | 289 | 36.975 | 9.258 | 75.303 | 1.00 | 23.27 | B |
| ATOM | 4630 | CA | ASN | 289 | 36.172 | 9.492 | 74.116 | 1.00 | 19.88 | B |
| ATOM | 4631 | CB | ASN | 289 | 36.898 | 8.993 | 72.871 | 1.00 | 18.84 | B |
| ATOM | 4632 | CG | ASN | 289 | 36.379 | 9.622 | 71.601 | 1.00 | 19.35 | B |
| ATOM | 4633 | OD1 | ASN | 289 | 37.155 | 10.094 | 70.786 | 1.00 | 21.16 | B |
| ATOM | 4634 | ND2 | ASN | 289 | 35.065 | 9.612 | 71.415 | 1.00 | 18.98 | B |
| ATOM | 4635 | C | ASN | 289 | 34.829 | 8.805 | 74.326 | 1.00 | 18.28 | B |
| ATOM | 4636 | O | ASN | 289 | 34.628 | 7.609 | 74.013 | 1.00 | 16.89 | B |
| ATOM | 4637 | N | GLN | 290 | 33.906 | 9.579 | 74.884 | 1.00 | 16.97 | B |
| ATOM | 4638 | CA | GLN | 290 | 32.560 | 9.115 | 75.178 | 1.00 | 14.08 | B |
| ATOM | 4639 | CB | GLN | 290 | 31.741 | 10.277 | 75.738 | 1.00 | 15.20 | B |
| ATOM | 4640 | CG | GLN | 290 | 30.328 | 9.905 | 76.161 | 1.00 | 16.32 | B |
| ATOM | 4641 | CD | GLN | 290 | 30.274 | 8.855 | 77.266 | 1.00 | 16.30 | B |
| ATOM | 4642 | OE1 | GLN | 290 | 29.232 | 8.273 | 77.512 | 1.00 | 16.57 | B |
| ATOM | 4643 | NE2 | GLN | 290 | 31.401 | 8.621 | 77.934 | 1.00 | 17.40 | B |
| ATOM | 4644 | C | GLN | 290 | 31.856 | 8.520 | 73.959 | 1.00 | 12.46 | B |
| ATOM | 4645 | O | GLN | 290 | 31.207 | 7.500 | 74.055 | 1.00 | 12.26 | B |
| ATOM | 4646 | N | SER | 291 | 31.971 | 9.174 | 72.814 | 1.00 | 11.04 | B |
| ATOM | 4647 | CA | SER | 291 | 31.333 | 8.627 | 71.629 | 1.00 | 11.96 | B |
| ATOM | 4648 | CB | SER | 291 | 31.404 | 9.609 | 70.466 | 1.00 | 11.35 | B |
| ATOM | 4649 | OG | SER | 291 | 30.393 | 10.586 | 70.582 | 1.00 | 12.37 | B |
| ATOM | 4650 | C | SER | 291 | 31.950 | 7.299 | 71.201 | 1.00 | 11.18 | B |
| ATOM | 4651 | O | SER | 291 | 31.241 | 6.375 | 70.783 | 1.00 | 11.32 | B |
| ATOM | 4652 | N | LEU | 292 | 33.270 | 7.205 | 71.294 | 1.00 | 11.69 | B |
| ATOM | 4653 | CA | LEU | 292 | 33.965 | 5.984 | 70.919 | 1.00 | 11.36 | B |
| ATOM | 4654 | CB | LEU | 292 | 35.485 | 6.237 | 70.902 | 1.00 | 9.67 | B |
| ATOM | 4655 | CG | LEU | 292 | 36.263 | 5.054 | 70.334 | 1.00 | 10.97 | B |
| ATOM | 4656 | CD1 | LEU | 292 | 35.817 | 4.822 | 68.911 | 1.00 | 10.21 | B |
| ATOM | 4657 | CD2 | LEU | 292 | 37.750 | 5.328 | 70.387 | 1.00 | 13.35 | B |
| ATOM | 4658 | C | LEU | 292 | 33.574 | 4.877 | 71.914 | 1.00 | 11.82 | B |
| ATOM | 4659 | O | LEU | 292 | 33.287 | 3.724 | 71.527 | 1.00 | 11.11 | B |
| ATOM | 4660 | N | LEU | 293 | 33.547 | 5.232 | 73.194 | 1.00 | 8.02 | B |
| ATOM | 4661 | CA | LEU | 293 | 33.210 | 4.295 | 74.246 | 1.00 | 7.35 | B |
| ATOM | 4662 | CB | LEU | 293 | 33.313 | 5.005 | 75.596 | 1.00 | 5.38 | B |
| ATOM | 4663 | CG | LEU | 293 | 34.410 | 4.587 | 76.570 | 1.00 | 6.04 | B |
| ATOM | 4664 | CD1 | LEU | 293 | 35.605 | 3.981 | 75.841 | 1.00 | 3.22 | B |
| ATOM | 4665 | CD2 | LEU | 293 | 34.798 | 5.808 | 77.389 | 1.00 | 3.25 | B |
| ATOM | 4666 | C | LEU | 293 | 31.802 | 3.747 | 74.071 | 1.00 | 7.33 | B |
| ATOM | 4667 | O | LEU | 293 | 31.563 | 2.550 | 74.222 | 1.00 | 9.04 | B |
| ATOM | 4668 | N | THR | 294 | 30.874 | 4.646 | 73.775 | 1.00 | 8.36 | B |
| ATOM | 4669 | CA | THR | 294 | 29.481 | 4.283 | 73.604 | 1.00 | 6.48 | B |
| ATOM | 4670 | CB | THR | 294 | 28.623 | 5.535 | 73.600 | 1.00 | 5.81 | B |
| ATOM | 4671 | OG1 | THR | 294 | 28.889 | 6.251 | 74.804 | 1.00 | 6.32 | B |
| ATOM | 4672 | CG2 | THR | 294 | 27.142 | 5.206 | 73.570 | 1.00 | 4.45 | B |
| ATOM | 4673 | C | THR | 294 | 29.237 | 3.461 | 72.364 | 1.00 | 7.94 | B |
| ATOM | 4674 | O | THR | 294 | 28.357 | 2.602 | 72.368 | 1.00 | 9.76 | B |
| ATOM | 4675 | N | LEU | 295 | 30.016 | 3.706 | 71.310 | 1.00 | 6.67 | B |
| ATOM | 4676 | CA | LEU | 295 | 29.896 | 2.918 | 70.074 | 1.00 | 6.68 | B |
| ATOM | 4677 | CB | LEU | 295 | 30.931 | 3.313 | 69.016 | 1.00 | 6.59 | B |
| ATOM | 4678 | CG | LEU | 295 | 30.897 | 2.510 | 67.708 | 1.00 | 5.44 | B |
| ATOM | 4679 | CD1 | LEU | 295 | 29.555 | 2.668 | 67.036 | 1.00 | 4.15 | B |
| ATOM | 4680 | CD2 | LEU | 295 | 31.969 | 2.993 | 66.786 | 1.00 | 5.26 | B |
| ATOM | 4681 | C | LEU | 295 | 30.228 | 1.473 | 70.403 | 1.00 | 8.24 | B |
| ATOM | 4682 | O | LEU | 295 | 29.615 | 0.555 | 69.887 | 1.00 | 9.80 | B |
| ATOM | 4683 | N | GLY | 296 | 31.214 | 1.290 | 71.276 | 1.00 | 9.60 | B |
| ATOM | 4684 | CA | GLY | 296 | 31.611 | −0.047 | 71.669 | 1.00 | 10.99 | B |
| ATOM | 4685 | C | GLY | 296 | 30.551 | −0.728 | 72.518 | 1.00 | 12.56 | B |
| ATOM | 4686 | O | GLY | 296 | 30.275 | −1.924 | 72.350 | 1.00 | 12.84 | B |
| ATOM | 4687 | N | ARG | 297 | 29.954 | 0.037 | 73.426 | 1.00 | 12.22 | B |
| ATOM | 4688 | CA | ARG | 297 | 28.928 | −0.486 | 74.307 | 1.00 | 12.41 | B |
| ATOM | 4689 | CB | ARG | 297 | 28.692 | 0.466 | 75.478 | 1.00 | 11.73 | B |
| ATOM | 4690 | CG | ARG | 297 | 29.818 | 0.493 | 76.498 | 1.00 | 10.69 | B |
| ATOM | 4691 | CD | ARG | 297 | 29.767 | 1.736 | 77.378 | 1.00 | 11.84 | B |
| ATOM | 4692 | NE | ARG | 297 | 30.969 | 1.856 | 78.205 | 1.00 | 10.74 | B |
| ATOM | 4693 | CZ | ARG | 297 | 31.409 | 2.993 | 78.734 | 1.00 | 10.49 | B |
| ATOM | 4694 | NH1 | ARG | 297 | 30.743 | 4.119 | 78.517 | 1.00 | 11.64 | B |
| ATOM | 4695 | NH2 | ARG | 297 | 32.504 | 3.003 | 79.486 | 1.00 | 9.73 | B |
| ATOM | 4696 | C | ARG | 297 | 27.622 | −0.708 | 73.569 | 1.00 | 13.86 | B |
| ATOM | 4697 | O | ARG | 297 | 26.798 | −1.514 | 74.009 | 1.00 | 13.06 | B |
| ATOM | 4698 | N | VAL | 298 | 27.426 | 0.014 | 72.464 | 1.00 | 14.33 | B |
| ATOM | 4699 | CA | VAL | 298 | 26.216 | −0.134 | 71.659 | 1.00 | 16.21 | B |
| ATOM | 4700 | CB | VAL | 298 | 26.048 | 1.031 | 70.696 | 1.00 | 16.05 | B |
| ATOM | 4701 | CG1 | VAL | 298 | 25.021 | 0.679 | 69.639 | 1.00 | 17.88 | B |
| ATOM | 4702 | CG2 | VAL | 298 | 25.605 | 2.257 | 71.458 | 1.00 | 18.13 | B |
| ATOM | 4703 | C | VAL | 298 | 26.281 | −1.426 | 70.853 | 1.00 | 17.16 | B |
| ATOM | 4704 | O | VAL | 298 | 25.305 | −2.173 | 70.774 | 1.00 | 18.74 | B |
| ATOM | 4705 | N | ILE | 299 | 27.441 | −1.691 | 70.262 | 1.00 | 18.24 | B |
| ATOM | 4706 | CA | ILE | 299 | 27.645 | −2.910 | 69.486 | 1.00 | 18.96 | B |
| ATOM | 4707 | CB | ILE | 299 | 29.019 | −2.868 | 68.770 | 1.00 | 19.68 | B |
| ATOM | 4708 | CG2 | ILE | 299 | 29.368 | −4.245 | 68.184 | 1.00 | 17.64 | B |
| ATOM | 4709 | CG1 | ILE | 299 | 28.983 | −1.791 | 67.674 | 1.00 | 19.70 | B |
| ATOM | 4710 | CD1 | ILE | 299 | 30.314 | −1.589 | 66.977 | 1.00 | 22.74 | B |
| ATOM | 4711 | C | ILE | 299 | 27.551 | −4.142 | 70.400 | 1.00 | 19.56 | B |
| ATOM | 4712 | O | ILE | 299 | 27.027 | −5.191 | 70.012 | 1.00 | 19.03 | B |
| ATOM | 4713 | N | THR | 300 | 28.043 | −4.017 | 71.624 | 1.00 | 19.86 | B |
| ATOM | 4714 | CA | THR | 300 | 27.978 | −5.136 | 72.551 | 1.00 | 20.92 | B |
| ATOM | 4715 | CB | THR | 300 | 28.770 | −4.841 | 73.824 | 1.00 | 20.58 | B |
| ATOM | 4716 | OG1 | THR | 300 | 30.172 | −4.893 | 73.533 | 1.00 | 21.97 | B |
| ATOM | 4717 | CG2 | THR | 300 | 28.433 | −5.845 | 74.903 | 1.00 | 21.65 | B |
| ATOM | 4718 | C | THR | 300 | 26.525 | −5.450 | 72.915 | 1.00 | 21.71 | B |
| ATOM | 4719 | O | THR | 300 | 26.134 | −6.601 | 72.984 | 1.00 | 22.71 | B |
| ATOM | 4720 | N | ALA | 301 | 25.728 | −4.413 | 73.139 | 1.00 | 23.13 | B |
| ATOM | 4721 | CA | ALA | 301 | 24.337 | −4.624 | 73.494 | 1.00 | 23.01 | B |
| ATOM | 4722 | CB | ALA | 301 | 23.694 | −3.327 | 73.904 | 1.00 | 22.73 | B |
| ATOM | 4723 | C | ALA | 301 | 23.589 | −5.225 | 72.323 | 1.00 | 23.48 | B |
| ATOM | 4724 | O | ALA | 301 | 22.652 | −5.982 | 72.509 | 1.00 | 23.63 | B |
| ATOM | 4725 | N | LEU | 302 | 24.005 | −4.872 | 71.111 | 1.00 | 23.21 | B |
| ATOM | 4726 | CA | LEU | 302 | 23.361 | −5.392 | 69.911 | 1.00 | 24.59 | B |
| ATOM | 4727 | CB | LEU | 302 | 23.737 | −4.526 | 68.695 | 1.00 | 23.93 | B |
| ATOM | 4728 | CG | LEU | 302 | 22.774 | −3.511 | 68.059 | 1.00 | 22.99 | B |
| ATOM | 4729 | CD1 | LEU | 302 | 21.827 | −2.952 | 69.058 | 1.00 | 20.71 | B |
| ATOM | 4730 | CD2 | LEU | 302 | 23.579 | −2.394 | 67.440 | 1.00 | 21.49 | B |
| ATOM | 4731 | C | LEU | 302 | 23.728 | −6.861 | 69.656 | 1.00 | 25.70 | B |
| ATOM | 4732 | O | LEU | 302 | 22.847 | −7.695 | 69.406 | 1.00 | 24.83 | B |
| ATOM | 4733 | N | VAL | 303 | 25.021 | −7.170 | 69.731 | 1.00 | 27.74 | B |
| ATOM | 4734 | CA | VAL | 303 | 25.527 | −8.521 | 69.505 | 1.00 | 29.35 | B |
| ATOM | 4735 | CB | VAL | 303 | 27.054 | −8.549 | 69.593 | 1.00 | 29.55 | B |
| ATOM | 4736 | CG1 | VAL | 303 | 27.545 | −9.975 | 69.439 | 1.00 | 30.49 | B |
| ATOM | 4737 | CG2 | VAL | 303 | 27.651 | −7.641 | 68.524 | 1.00 | 30.24 | B |
| ATOM | 4738 | C | VAL | 303 | 24.985 | −9.528 | 70.510 | 1.00 | 31.00 | B |
| ATOM | 4739 | O | VAL | 303 | 24.629 | −10.631 | 70.160 | 1.00 | 30.43 | B |
| ATOM | 4740 | N | GLU | 304 | 24.927 | −9.123 | 71.770 | 1.00 | 33.86 | B |
| ATOM | 4741 | CA | GLU | 304 | 24.442 | −9.986 | 72.838 | 1.00 | 36.40 | B |
| ATOM | 4742 | CB | GLU | 304 | 25.130 | −9.594 | 74.143 | 1.00 | 37.33 | B |
| ATOM | 4743 | CG | GLU | 304 | 26.650 | −9.690 | 74.076 | 1.00 | 39.18 | B |
| ATOM | 4744 | CD | GLU | 304 | 27.316 | −9.437 | 75.422 | 1.00 | 41.19 | B |
| ATOM | 4745 | OE1 | GLU | 304 | 28.564 | −9.473 | 75.490 | 1.00 | 42.27 | B |
| ATOM | 4746 | OE2 | GLU | 304 | 26.594 | −9.202 | 76.413 | 1.00 | 42.10 | B |
| ATOM | 4747 | C | GLU | 304 | 22.922 | −9.924 | 72.985 | 1.00 | 38.11 | B |
| ATOM | 4748 | O | GLU | 304 | 22.334 | −10.552 | 73.871 | 1.00 | 37.60 | B |
| ATOM | 4749 | N | ARG | 305 | 22.303 | −9.155 | 72.098 | 1.00 | 41.03 | B |
| ATOM | 4750 | CA | ARG | 305 | 20.860 | −8.996 | 72.068 | 1.00 | 43.26 | B |
| ATOM | 4751 | CB | ARG | 305 | 20.221 | −10.302 | 71.592 | 1.00 | 44.67 | B |
| ATOM | 4752 | CG | ARG | 305 | 20.602 | −10.629 | 70.151 | 1.00 | 46.86 | B |
| ATOM | 4753 | CD | ARG | 305 | 20.167 | −12.025 | 69.716 | 1.00 | 49.68 | B |
| ATOM | 4754 | NE | ARG | 305 | 20.654 | −12.350 | 68.373 | 1.00 | 50.79 | B |
| ATOM | 4755 | CZ | ARG | 305 | 20.244 | −11.753 | 67.258 | 1.00 | 50.97 | B |
| ATOM | 4756 | NH1 | ARG | 305 | 19.327 | −10.797 | 67.309 | 1.00 | 51.47 | B |
| ATOM | 4757 | NH2 | ARG | 305 | 20.769 | −12.097 | 66.089 | 1.00 | 51.54 | B |
| ATOM | 4758 | C | ARG | 305 | 20.237 | −8.514 | 73.367 | 1.00 | 43.49 | B |
| ATOM | 4759 | O | ARG | 305 | 19.142 | −8.909 | 73.718 | 1.00 | 44.11 | B |
| ATOM | 4760 | N | THR | 306 | 20.951 | −7.648 | 74.077 | 1.00 | 44.17 | B |
| ATOM | 4761 | CA | THR | 306 | 20.444 | −7.078 | 75.319 | 1.00 | 43.76 | B |
| ATOM | 4762 | CB | THR | 306 | 21.535 | −6.267 | 76.040 | 1.00 | 43.72 | B |
| ATOM | 4763 | OG1 | THR | 306 | 22.623 | −7.131 | 76.399 | 1.00 | 43.84 | B |
| ATOM | 4764 | CG2 | THR | 306 | 20.975 | −5.602 | 77.288 | 1.00 | 43.30 | B |
| ATOM | 4765 | C | THR | 306 | 19.307 | −6.139 | 74.912 | 1.00 | 44.17 | B |
| ATOM | 4766 | O | THR | 306 | 19.388 | −5.459 | 73.891 | 1.00 | 45.09 | B |
| ATOM | 4767 | N | PRO | 307 | 18.226 | −6.098 | 75.700 | 1.00 | 43.54 | B |
| ATOM | 4768 | CD | PRO | 307 | 17.925 | −6.973 | 76.846 | 1.00 | 43.66 | B |
| ATOM | 4769 | CA | PRO | 307 | 17.080 | −5.232 | 75.390 | 1.00 | 42.75 | B |
| ATOM | 4770 | CB | PRO | 307 | 16.101 | −5.554 | 76.518 | 1.00 | 43.35 | B |
| ATOM | 4771 | CG | PRO | 307 | 16.429 | −7.001 | 76.834 | 1.00 | 44.16 | B |
| ATOM | 4772 | C | PRO | 307 | 17.408 | −3.741 | 75.269 | 1.00 | 41.65 | B |
| ATOM | 4773 | O | PRO | 307 | 16.903 | −3.049 | 74.384 | 1.00 | 41.15 | B |
| ATOM | 4774 | N | HIS | 308 | 18.254 | −3.247 | 76.166 | 1.00 | 39.72 | B |
| ATOM | 4775 | CA | HIS | 308 | 18.629 | −1.839 | 76.164 | 1.00 | 37.51 | B |
| ATOM | 4776 | CB | HIS | 308 | 18.774 | −1.336 | 77.587 | 1.00 | 39.81 | B |
| ATOM | 4777 | CG | HIS | 308 | 19.193 | 0.097 | 77.677 | 1.00 | 42.26 | B |
| ATOM | 4778 | CD2 | HIS | 308 | 20.336 | 0.664 | 78.127 | 1.00 | 43.26 | B |
| ATOM | 4779 | ND1 | HIS | 308 | 18.391 | 1.131 | 77.247 | 1.00 | 43.54 | B |
| ATOM | 4780 | CE1 | HIS | 308 | 19.024 | 2.278 | 77.428 | 1.00 | 44.49 | B |
| ATOM | 4781 | NE2 | HIS | 308 | 20.205 | 2.024 | 77.959 | 1.00 | 44.29 | B |
| ATOM | 4782 | C | HIS | 308 | 19.937 | −1.559 | 75.446 | 1.00 | 35.63 | B |
| ATOM | 4783 | O | HIS | 308 | 20.958 | −2.160 | 75.745 | 1.00 | 36.69 | B |
| ATOM | 4784 | N | VAL | 309 | 19.889 | −0.627 | 74.501 | 1.00 | 32.04 | B |
| ATOM | 4785 | CA | VAL | 309 | 21.071 | −0.237 | 73.731 | 1.00 | 27.44 | B |
| ATOM | 4786 | CB | VAL | 309 | 20.821 | −0.415 | 72.218 | 1.00 | 27.23 | B |
| ATOM | 4787 | CG1 | VAL | 309 | 22.090 | −0.111 | 71.426 | 1.00 | 27.83 | B |
| ATOM | 4788 | CG2 | VAL | 309 | 20.336 | −1.823 | 71.946 | 1.00 | 25.00 | B |
| ATOM | 4789 | C | VAL | 309 | 21.307 | 1.234 | 74.059 | 1.00 | 26.45 | B |
| ATOM | 4790 | O | VAL | 309 | 20.501 | 2.090 | 73.724 | 1.00 | 26.41 | B |
| ATOM | 4791 | N | PRO | 310 | 22.432 | 1.538 | 74.715 | 1.00 | 25.12 | B |
| ATOM | 4792 | CD | PRO | 310 | 23.508 | 0.587 | 75.062 | 1.00 | 23.57 | B |
| ATOM | 4793 | CA | PRO | 310 | 22.780 | 2.914 | 75.107 | 1.00 | 22.73 | B |
| ATOM | 4794 | CB | PRO | 310 | 23.985 | 2.701 | 76.007 | 1.00 | 23.56 | B |
| ATOM | 4795 | CG | PRO | 310 | 24.671 | 1.504 | 75.354 | 1.00 | 23.96 | B |
| ATOM | 4796 | C | PRO | 310 | 23.017 | 3.958 | 73.999 | 1.00 | 22.22 | B |
| ATOM | 4797 | O | PRO | 310 | 23.965 | 4.735 | 74.073 | 1.00 | 21.14 | B |
| ATOM | 4798 | N | TYR | 311 | 22.147 | 4.000 | 72.995 | 1.00 | 21.70 | B |
| ATOM | 4799 | CA | TYR | 311 | 22.294 | 4.967 | 71.899 | 1.00 | 22.33 | B |
| ATOM | 4800 | CB | TYR | 311 | 21.083 | 4.978 | 70.970 | 1.00 | 22.30 | B |
| ATOM | 4801 | CG | TYR | 311 | 20.861 | 3.721 | 70.154 | 1.00 | 24.68 | B |
| ATOM | 4802 | CD1 | TYR | 311 | 21.773 | 3.322 | 69.177 | 1.00 | 25.08 | B |
| ATOM | 4803 | CE1 | TYR | 311 | 21.555 | 2.171 | 68.411 | 1.00 | 25.18 | B |
| ATOM | 4804 | CD2 | TYR | 311 | 19.717 | 2.937 | 70.347 | 1.00 | 24.09 | B |
| ATOM | 4805 | CE2 | TYR | 311 | 19.493 | 1.786 | 69.590 | 1.00 | 24.09 | B |
| ATOM | 4806 | CZ | TYR | 311 | 20.416 | 1.405 | 68.623 | 1.00 | 24.98 | B |
| ATOM | 4807 | OH | TYR | 311 | 20.211 | 0.246 | 67.893 | 1.00 | 24.66 | B |
| ATOM | 4808 | C | TYR | 311 | 22.431 | 6.429 | 72.338 | 1.00 | 21.98 | B |
| ATOM | 4809 | O | TYR | 311 | 23.180 | 7.188 | 71.741 | 1.00 | 23.57 | B |
| ATOM | 4810 | N | ARG | 312 | 21.707 | 6.813 | 73.384 | 1.00 | 20.49 | B |
| ATOM | 4811 | CA | ARG | 312 | 21.726 | 8.203 | 73.861 | 1.00 | 19.38 | B |
| ATOM | 4812 | CB | ARG | 312 | 20.447 | 8.544 | 74.640 | 1.00 | 21.56 | B |
| ATOM | 4813 | CG | ARG | 312 | 19.150 | 8.149 | 73.951 | 1.00 | 24.98 | B |
| ATOM | 4814 | CD | ARG | 312 | 17.949 | 8.887 | 74.534 | 1.00 | 27.94 | B |
| ATOM | 4815 | NE | ARG | 312 | 16.688 | 8.240 | 74.175 | 1.00 | 31.63 | B |
| ATOM | 4816 | CZ | ARG | 312 | 16.262 | 7.086 | 74.688 | 1.00 | 34.10 | B |
| ATOM | 4817 | NH1 | ARG | 312 | 16.996 | 6.445 | 75.590 | 1.00 | 37.15 | B |
| ATOM | 4818 | NH2 | ARG | 312 | 15.101 | 6.566 | 74.304 | 1.00 | 33.60 | B |
| ATOM | 4819 | C | ARG | 312 | 22.875 | 8.612 | 74.779 | 1.00 | 17.27 | B |
| ATOM | 4820 | O | ARG | 312 | 22.933 | 9.756 | 75.235 | 1.00 | 16.64 | B |
| ATOM | 4821 | N | GLU | 313 | 23.786 | 7.686 | 75.054 | 1.00 | 14.25 | B |
| ATOM | 4822 | CA | GLU | 313 | 24.908 | 7.986 | 75.935 | 1.00 | 11.55 | B |
| ATOM | 4823 | CB | GLU | 313 | 25.410 | 6.693 | 76.590 | 1.00 | 11.14 | B |
| ATOM | 4824 | CG | GLU | 313 | 24.416 | 6.136 | 77.618 | 1.00 | 11.41 | B |
| ATOM | 4825 | CD | GLU | 313 | 24.916 | 4.905 | 78.379 | 1.00 | 12.57 | B |
| ATOM | 4826 | OE1 | GLU | 313 | 26.071 | 4.898 | 78.834 | 1.00 | 11.41 | B |
| ATOM | 4827 | OE2 | GLU | 313 | 24.149 | 3.935 | 78.569 | 1.00 | 14.80 | B |
| ATOM | 4828 | C | GLU | 313 | 26.053 | 8.746 | 75.271 | 1.00 | 10.23 | B |
| ATOM | 4829 | O | GLU | 313 | 27.066 | 8.960 | 75.891 | 1.00 | 10.15 | B |
| ATOM | 4830 | N | SER | 314 | 25.865 | 9.164 | 74.017 | 1.00 | 10.36 | B |
| ATOM | 4831 | CA | SER | 314 | 26.878 | 9.912 | 73.263 | 1.00 | 9.41 | B |
| ATOM | 4832 | CB | SER | 314 | 28.000 | 9.018 | 72.732 | 1.00 | 10.81 | B |
| ATOM | 4833 | OG | SER | 314 | 27.643 | 8.320 | 71.544 | 1.00 | 9.64 | B |
| ATOM | 4834 | C | SER | 314 | 26.235 | 10.511 | 72.031 | 1.00 | 10.05 | B |
| ATOM | 4835 | O | SER | 314 | 25.190 | 10.052 | 71.583 | 1.00 | 9.18 | B |
| ATOM | 4836 | N | LYS | 315 | 26.887 | 11.544 | 71.501 | 1.00 | 10.81 | B |
| ATOM | 4837 | CA | LYS | 315 | 26.428 | 12.259 | 70.320 | 1.00 | 9.07 | B |
| ATOM | 4838 | CB | LYS | 315 | 27.254 | 13.527 | 70.063 | 1.00 | 9.50 | B |
| ATOM | 4839 | CG | LYS | 315 | 27.390 | 14.463 | 71.236 | 1.00 | 9.25 | B |
| ATOM | 4840 | CD | LYS | 315 | 26.058 | 14.973 | 71.686 | 1.00 | 10.89 | B |
| ATOM | 4841 | CE | LYS | 315 | 26.244 | 16.156 | 72.620 | 1.00 | 13.02 | B |
| ATOM | 4842 | NZ | LYS | 315 | 26.918 | 17.316 | 71.937 | 1.00 | 14.10 | B |
| ATOM | 4843 | C | LYS | 315 | 26.556 | 11.414 | 69.077 | 1.00 | 8.68 | B |
| ATOM | 4844 | O | LYS | 315 | 25.652 | 11.383 | 68.282 | 1.00 | 10.14 | B |
| ATOM | 4845 | N | LEU | 316 | 27.683 | 10.721 | 68.931 | 1.00 | 8.28 | B |
| ATOM | 4846 | CA | LEU | 316 | 27.928 | 9.888 | 67.763 | 1.00 | 7.48 | B |
| ATOM | 4847 | CB | LEU | 316 | 29.297 | 9.205 | 67.867 | 1.00 | 6.90 | B |
| ATOM | 4848 | CG | LEU | 316 | 29.679 | 8.277 | 66.713 | 1.00 | 8.06 | B |
| ATOM | 4849 | CD1 | LEU | 316 | 30.018 | 9.097 | 65.484 | 1.00 | 10.24 | B |
| ATOM | 4850 | CD2 | LEU | 316 | 30.850 | 7.452 | 67.129 | 1.00 | 8.22 | B |
| ATOM | 4851 | C | LEU | 316 | 26.852 | 8.821 | 67.590 | 1.00 | 9.38 | B |
| ATOM | 4852 | O | LEU | 316 | 26.241 | 8.733 | 66.523 | 1.00 | 9.82 | B |
| ATOM | 4853 | N | THR | 317 | 26.588 | 8.040 | 68.642 | 1.00 | 9.80 | B |
| ATOM | 4854 | CA | THR | 317 | 25.599 | 6.965 | 68.534 | 1.00 | 10.18 | B |
| ATOM | 4855 | CB | THR | 317 | 25.672 | 5.952 | 69.674 | 1.00 | 10.15 | B |
| ATOM | 4856 | OG1 | THR | 317 | 25.527 | 6.642 | 70.909 | 1.00 | 10.81 | B |
| ATOM | 4857 | CG2 | THR | 317 | 27.004 | 5.185 | 69.661 | 1.00 | 9.59 | B |
| ATOM | 4858 | C | THR | 317 | 24.175 | 7.455 | 68.484 | 1.00 | 10.03 | B |
| ATOM | 4859 | O | THR | 317 | 23.295 | 6.709 | 68.146 | 1.00 | 11.71 | B |
| ATOM | 4860 | N | ARG | 318 | 23.947 | 8.703 | 68.867 | 1.00 | 9.69 | B |
| ATOM | 4861 | CA | ARG | 318 | 22.607 | 9.256 | 68.785 | 1.00 | 9.04 | B |
| ATOM | 4862 | CB | ARG | 318 | 22.454 | 10.464 | 69.703 | 1.00 | 13.23 | B |
| ATOM | 4863 | CG | ARG | 318 | 21.719 | 10.147 | 71.004 | 1.00 | 19.08 | B |
| ATOM | 4864 | CD | ARG | 318 | 22.058 | 11.133 | 72.115 | 1.00 | 22.73 | B |
| ATOM | 4865 | NE | ARG | 318 | 21.617 | 12.495 | 71.828 | 1.00 | 26.31 | B |
| ATOM | 4866 | CZ | ARG | 318 | 20.345 | 12.863 | 71.705 | 1.00 | 27.29 | B |
| ATOM | 4867 | NH1 | ARG | 318 | 19.383 | 11.963 | 71.849 | 1.00 | 28.67 | B |
| ATOM | 4868 | NH2 | ARG | 318 | 20.036 | 14.124 | 71.429 | 1.00 | 25.94 | B |
| ATOM | 4869 | C | ARG | 318 | 22.434 | 9.679 | 67.344 | 1.00 | 8.51 | B |
| ATOM | 4870 | O | ARG | 318 | 21.418 | 9.412 | 66.720 | 1.00. | 10.84 | B |
| ATOM | 4871 | N | ILE | 319 | 23.445 | 10.339 | 66.799 | 1.00 | 5.66 | B |
| ATOM | 4872 | CA | ILE | 319 | 23.352 | 10.766 | 65.410 | 1.00 | 5.05 | B |
| ATOM | 4873 | CB | ILE | 319 | 24.591 | 11.627 | 65.014 | 1.00 | 5.19 | B |
| ATOM | 4874 | CG2 | ILE | 319 | 24.531 | 11.976 | 63.544 | 1.00 | 6.51 | B |
| ATOM | 4875 | CG1 | ILE | 319 | 24.603 | 12.935 | 65.826 | 1.00 | 5.47 | B |
| ATOM | 4876 | CD1 | ILE | 319 | 25.833 | 13.774 | 65.632 | 1.00 | 2.71 | B |
| ATOM | 4877 | C | ILE | 319 | 23.227 | 9.551 | 64.460 | 1.00 | 3.03 | B |
| ATOM | 4878 | O | ILE | 319 | 22.361 | 9.511 | 63.590 | 1.00 | 1.95 | B |
| ATOM | 4879 | N | LEU | 320 | 24.067 | 8.540 | 64.657 | 1.00 | 4.41 | B |
| ATOM | 4880 | CA | LEU | 320 | 24.056 | 7.376 | 63.767 | 1.00 | 5.60 | B |
| ATOM | 4881 | CB | LEU | 320 | 25.490 | 6.931 | 63.451 | 1.00 | 2.81 | B |
| ATOM | 4882 | CG | LEU | 320 | 26.437 | 7.964 | 62.845 | 1.00 | 2.57 | B |
| ATOM | 4883 | CD1 | LEU | 320 | 27.873 | 7.442 | 62.786 | 1.00 | 2.20 | B |
| ATOM | 4884 | CD2 | LEU | 320 | 25.955 | 8.334 | 61.476 | 1.00 | 1.00 | B |
| ATOM | 4885 | C | LEU | 320 | 23.313 | 6.122 | 64.235 | 1.00 | 7.52 | B |
| ATOM | 4886 | O | LEU | 320 | 23.620 | 5.045 | 63.776 | 1.00 | 7.94 | B |
| ATOM | 4887 | N | GLN | 321 | 22.306 | 6.258 | 65.094 | 1.00 | 10.60 | B |
| ATOM | 4888 | CA | GLN | 321 | 21.629 | 5.057 | 65.604 | 1.00 | 16.44 | B |
| ATOM | 4889 | CB | GLN | 321 | 20.679 | 5.362 | 66.775 | 1.00 | 18.94 | B |
| ATOM | 4890 | CG | GLN | 321 | 19.433 | 6.153 | 66.458 | 1.00 | 22.43 | B |
| ATOM | 4891 | CD | GLN | 321 | 18.593 | 6.391 | 67.707 | 1.00 | 25.16 | B |
| ATOM | 4892 | OE1 | GLN | 321 | 18.121 | 5.453 | 68.338 | 1.00 | 26.09 | B |
| ATOM | 4893 | NE2 | GLN | 321 | 18.418 | 7.658 | 68.071 | 1.00 | 26.05 | B |
| ATOM | 4894 | C | GLN | 321 | 20.882 | 4.186 | 64.617 | 1.00 | 16.64 | B |
| ATOM | 4895 | O | GLN | 321 | 20.700 | 2.992 | 64.870 | 1.00 | 16.23 | B |
| ATOM | 4896 | N | ASP | 322 | 20.439 | 4.759 | 63.505 | 1.00 | 17.01 | B |
| ATOM | 4897 | CA | ASP | 322 | 19.762 | 3.931 | 62.521 | 1.00 | 19.03 | B |
| ATOM | 4898 | CB | ASP | 322 | 18.952 | 4.755 | 61.535 | 1.00 | 20.75 | B |
| ATOM | 4899 | CG | ASP | 322 | 17.983 | 3.896 | 60.727 | 1.00 | 22.50 | B |
| ATOM | 4900 | OD1 | ASP | 322 | 17.835 | 4.125 | 59.506 | 1.00 | 24.17 | B |
| ATOM | 4901 | OD2 | ASP | 322 | 17.352 | 2.997 | 61.327 | 1.00 | 21.00 | B |
| ATOM | 4902 | C | ASP | 322 | 20.803 | 3.139 | 61.722 | 1.00 | 20.46 | B |
| ATOM | 4903 | O | ASP | 322 | 20.467 | 2.335 | 60.861 | 1.00 | 23.04 | B |
| ATOM | 4904 | N | SER | 323 | 22.076 | 3.385 | 62.006 | 1.00 | 20.16 | B |
| ATOM | 4905 | CA | SER | 323 | 23.164 | 2.670 | 61.353 | 1.00 | 18.88 | B |
| ATOM | 4906 | CB | SER | 323 | 24.299 | 3.643 | 61.077 | 1.00 | 17.96 | B |
| ATOM | 4907 | OG | SER | 323 | 23.842 | 4.642 | 60.187 | 1.00 | 18.62 | B |
| ATOM | 4908 | C | SER | 323 | 23.625 | 1.518 | 62.259 | 1.00 | 18.52 | B |
| ATOM | 4909 | O | SER | 323 | 24.368 | 0.647 | 61.838 | 1.00 | 19.83 | B |
| ATOM | 4910 | N | LEU | 324 | 23.168 | 1.512 | 63.507 | 1.00 | 16.09 | B |
| ATOM | 4911 | CA | LEU | 324 | 23.541 | 0.449 | 64.420 | 1.00 | 16.61 | B |
| ATOM | 4912 | CB | LEU | 324 | 24.257 | 1.026 | 65.648 | 1.00 | 15.87 | B |
| ATOM | 4913 | CG | LEU | 324 | 25.679 | 1.595 | 65.539 | 1.00 | 14.59 | B |
| ATOM | 4914 | CD1 | LEU | 324 | 26.545 | 0.643 | 64.722 | 1.00 | 13.37 | B |
| ATOM | 4915 | CD2 | LEU | 324 | 25.649 | 2.965 | 64.909 | 1.00 | 11.67 | B |
| ATOM | 4916 | C | LEU | 324 | 22.300 | −0.343 | 64.834 | 1.00 | 17.48 | B |
| ATOM | 4917 | O | LEU | 324 | 21.651 | −0.025 | 65.814 | 1.00 | 16.83 | B |
| ATOM | 4918 | N | GLY | 325 | 21.983 | −1.387 | 64.071 | 1.00 | 17.97 | B |
| ATOM | 4919 | CA | GLY | 325 | 20.818 | −2.203 | 64.377 | 1.00 | 18.49 | B |
| ATOM | 4920 | C | GLY | 325 | 19.498 | −1.576 | 63.939 | 1.00 | 19.29 | B |
| ATOM | 4921 | O | GLY | 325 | 18.427 | −1.950 | 64.423 | 1.00 | 19.24 | B |
| ATOM | 4922 | N | GLY | 326 | 19.573 | −0.630 | 63.007 | 1.00 | 19.01 | B |
| ATOM | 4923 | CA | GLY | 326 | 18.382 | 0.052 | 62.539 | 1.00 | 18.79 | B |
| ATOM | 4924 | C | GLY | 326 | 17.935 | −0.373 | 61.165 | 1.00 | 19.04 | B |
| ATOM | 4925 | O | GLY | 326 | 17.931 | −1.550 | 60.861 | 1.00 | 18.81 | B |
| ATOM | 4926 | N | ARG | 327 | 17.565 | 0.603 | 60.341 | 1.00 | 19.26 | B |
| ATOM | 4927 | CA | ARG | 327 | 17.106 | 0.336 | 58.991 | 1.00 | 20.71 | B |
| ATOM | 4928 | CB | ARG | 327 | 15.731 | 0.970 | 58.761 | 1.00 | 22.28 | B |
| ATOM | 4929 | CG | ARG | 327 | 14.591 | 0.225 | 59.443 | 1.00 | 25.87 | B |
| ATOM | 4930 | CD | ARG | 327 | 13.233 | 0.703 | 58.976 | 1.00 | 28.38 | B |
| ATOM | 4931 | NE | ARG | 327 | 12.260 | −0.388 | 58.957 | 1.00 | 33.27 | B |
| ATOM | 4932 | CZ | ARG | 327 | 12.370 | −1.477 | 58.193 | 1.00 | 36.86 | B |
| ATOM | 4933 | NH1 | ARG | 327 | 13.412 | −1.639 | 57.382 | 1.00 | 38.23 | B |
| ATOM | 4934 | NH2 | ARG | 327 | 11.422 | −2.399 | 58.213 | 1.00 | 38.97 | B |
| ATOM | 4935 | C | ARG | 327 | 18.072 | 0.784 | 57.899 | 1.00 | 20.64 | B |
| ATOM | 4936 | O | ARG | 327 | 17.721 | 0.788 | 56.718 | 1.00 | 19.55 | B |
| ATOM | 4937 | N | THR | 328 | 19.295 | 1.127 | 58.293 | 1.00 | 19.88 | B |
| ATOM | 4938 | CA | THR | 328 | 20.316 | 1.568 | 57.349 | 1.00 | 18.38 | B |
| ATOM | 4939 | CB | THR | 328 | 21.133 | 2.694 | 57.948 | 1.00 | 16.59 | B |
| ATOM | 4940 | OG1 | THR | 328 | 20.260 | 3.780 | 58.254 | 1.00 | 15.01 | B |
| ATOM | 4941 | CG2 | THR | 328 | 22.170 | 3.171 | 56.975 | 1.00 | 16.39 | B |
| ATOM | 4942 | C | THR | 328 | 21.271 | 0.449 | 56.971 | 1.00 | 17.88 | B |
| ATOM | 4943 | O | THR | 328 | 21.640 | −0.343 | 57.808 | 1.00 | 18.85 | B |
| ATOM | 4944 | N | ARG | 329 | 21.659 | 0.380 | 55.701 | 1.00 | 18.85 | B |
| ATOM | 4945 | CA | ARG | 329 | 22.605 | −0.648 | 55.284 | 1.00 | 18.48 | B |
| ATOM | 4946 | CB | ARG | 329 | 22.644 | −0.784 | 53.756 | 1.00 | 21.31 | B |
| ATOM | 4947 | CG | ARG | 329 | 23.540 | −1.929 | 53.249 | 1.00 | 27.66 | B |
| ATOM | 4948 | CD | ARG | 329 | 23.818 | −1.771 | 51.748 | 1.00 | 32.45 | B |
| ATOM | 4949 | NE | ARG | 329 | 24.651 | −2.837 | 51.190 | 1.00 | 38.68 | B |
| ATOM | 4950 | CZ | ARG | 329 | 25.871 | −3.147 | 51.626 | 1.00 | 43.03 | B |
| ATOM | 4951 | NH1 | ARG | 329 | 26.417 | −2.476 | 52.641 | 1.00 | 45.11 | B |
| ATOM | 4952 | NH2 | ARG | 329 | 26.553 | −4.122 | 51.032 | 1.00 | 45.04 | B |
| ATOM | 4953 | C | ARG | 329 | 23.937 | −0.161 | 55.840 | 1.00 | 14.99 | B |
| ATOM | 4954 | O | ARG | 329 | 24.361 | 0.948 | 55.568 | 1.00 | 16.21 | B |
| ATOM | 4955 | N | THR | 330 | 24.595 | −0.987 | 56.632 | 1.00 | 12.23 | B |
| ATOM | 4956 | CA | THR | 330 | 25.842 | −0.559 | 57.235 | 1.00 | 11.36 | B |
| ATOM | 4957 | CB | THR | 330 | 25.720 | −0.515 | 58.801 | 1.00 | 11.85 | B |
| ATOM | 4958 | OG1 | THR | 330 | 24.663 | 0.378 | 59.185 | 1.00 | 12.21 | B |
| ATOM | 4959 | CG2 | THR | 330 | 27.022 | −0.038 | 59.432 | 1.00 | 10.17 | B |
| ATOM | 4960 | C | THR | 330 | 27.031 | −1.424 | 56.857 | 1.00 | 11.32 | B |
| ATOM | 4961 | O | THR | 330 | 26.909 | −2.639 | 56.699 | 1.00 | 11.14 | B |
| ATOM | 4962 | N | SER | 331 | 28.176 | −0.760 | 56.722 | 1.00 | 10.11 | B |
| ATOM | 4963 | CA | SER | 331 | 29.432 | −1.390 | 56.396 | 1.00 | 9.70 | B |
| ATOM | 4964 | CB | SER | 331 | 29.762 | −1.121 | 54.938 | 1.00 | 10.15 | B |
| ATOM | 4965 | OG | SER | 331 | 29.612 | −2.305 | 54.201 | 1.00 | 16.41 | B |
| ATOM | 4966 | C | SER | 331 | 30.551 | −0.861 | 57.292 | 1.00 | 8.79 | B |
| ATOM | 4967 | O | SER | 331 | 30.612 | 0.314 | 57.575 | 1.00 | 10.25 | B |
| ATOM | 4968 | N | ILE | 332 | 31.421 | −1.744 | 57.761 | 1.00 | 7.54 | B |
| ATOM | 4969 | CA | ILE | 332 | 32.537 | −1.309 | 58.580 | 1.00 | 5.00 | B |
| ATOM | 4970 | CB | ILE | 332 | 32.484 | −1.896 | 59.997 | 1.00 | 3.72 | B |
| ATOM | 4971 | CG2 | ILE | 332 | 33.791 | −1.623 | 60.719 | 1.00 | 1.00 | B |
| ATOM | 4972 | CG1 | ILE | 332 | 31.296 | −1.308 | 60.755 | 1.00 | 1.20 | B |
| ATOM | 4973 | CD1 | ILE | 332 | 31.044 | −1.996 | 62.080 | 1.00 | 1.00 | B |
| ATOM | 4974 | C | ILE | 332 | 33.825 | −1.761 | 57.915 | 1.00 | 6.57 | B |
| ATOM | 4975 | O | ILE | 332 | 33.959 | −2.921 | 57.505 | 1.00 | 6.08 | B |
| ATOM | 4976 | N | ILE | 333 | 34.754 | −0.824 | 57.779 | 1.00 | 6.74 | B |
| ATOM | 4977 | CA | ILE | 333 | 36.052 | −1.110 | 57.203 | 1.00 | 7.94 | B |
| ATOM | 4978 | CB | ILE | 333 | 36.377 | −0.134 | 56.043 | 1.00 | 7.86 | B |
| ATOM | 4979 | CG2 | ILE | 333 | 37.745 | −0.446 | 55.482 | 1.00 | 10.20 | B |
| ATOM | 4980 | CG1 | ILE | 333 | 35.335 | −0.292 | 54.935 | 1.00 | 9.26 | B |
| ATOM | 4981 | CD1 | ILE | 333 | 35.562 | 0.532 | 53.743 | 1.00 | 9.53 | B |
| ATOM | 4982 | C | ILE | 333 | 37.050 | −0.961 | 58.362 | 1.00 | 9.22 | B |
| ATOM | 4983 | O | ILE | 333 | 37.318 | 0.139 | 58.833 | 1.00 | 9.93 | B |
| ATOM | 4984 | N | ALA | 334 | 37.568 | −2.087 | 58.842 | 1.00 | 9.27 | B |
| ATOM | 4985 | CA | ALA | 334 | 38.510 | −2.064 | 59.950 | 1.00 | 9.36 | B |
| ATOM | 4986 | CB | ALA | 334 | 38.318 | −3.281 | 60.815 | 1.00 | 8.99 | B |
| ATOM | 4987 | C | ALA | 334 | 39.914 | −2.033 | 59.366 | 1.00 | 9.97 | B |
| ATOM | 4988 | O | ALA | 334 | 40.289 | −2.887 | 58.558 | 1.00 | 9.97 | B |
| ATOM | 4989 | N | THR | 335 | 40.689 | −1.039 | 59.780 | 1.00 | 10.59 | B |
| ATOM | 4990 | CA | THR | 335 | 42.041 | −0.877 | 59.267 | 1.00 | 11.33 | B |
| ATOM | 4991 | CB | THR | 335 | 42.300 | 0.587 | 58.833 | 1.00 | 11.54 | B |
| ATOM | 4992 | OG1 | THR | 335 | 42.165 | 1.471 | 59.959 | 1.00 | 11.31 | B |
| ATOM | 4993 | CG2 | THR | 335 | 41.316 | 0.973 | 57.707 | 1.00 | 10.89 | B |
| ATOM | 4994 | C | THR | 335 | 43.059 | −1.311 | 60.297 | 1.00 | 11.80 | B |
| ATOM | 4995 | O | THR | 335 | 42.898 | −1.044 | 61.479 | 1.00 | 11.91 | B |
| ATOM | 4996 | N | ILE | 336 | 44.108 | −1.981 | 59.825 | 1.00 | 10.99 | B |
| ATOM | 4997 | CA | ILE | 336 | 45.150 | −2.494 | 60.691 | 1.00 | 9.23 | B |
| ATOM | 4998 | CB | ILE | 336 | 44.988 | −4.002 | 60.867 | 1.00 | 6.21 | B |
| ATOM | 4999 | CG2 | ILE | 336 | 43.726 | −4.275 | 61.631 | 1.00 | 2.30 | B |
| ATOM | 5000 | CG1 | ILE | 336 | 44.949 | −4.688 | 59.501 | 1.00 | 4.99 | B |
| ATOM | 5001 | CD1 | ILE | 336 | 44.977 | −6.187 | 59.570 | 1.00 | 4.80 | B |
| ATOM | 5002 | C | ILE | 336 | 46.549 | −2.201 | 60.175 | 1.00 | 12.29 | B |
| ATOM | 5003 | O | ILE | 336 | 46.722 | −1.683 | 59.054 | 1.00 | 12.52 | B |
| ATOM | 5004 | N | SER | 337 | 47.536 | −2.533 | 61.011 | 1.00 | 15.10 | B |
| ATOM | 5005 | CA | SER | 337 | 48.958 | −2.344 | 60.716 | 1.00 | 17.38 | B |
| ATOM | 5006 | CB | SER | 337 | 49.673 | −1.619 | 61.848 | 1.00 | 16.32 | B |
| ATOM | 5007 | OG | SER | 337 | 51.071 | −1.842 | 61.757 | 1.00 | 15.90 | B |
| ATOM | 5008 | C | SER | 337 | 49.690 | −3.686 | 60.569 | 1.00 | 18.53 | B |
| ATOM | 5009 | O | SER | 337 | 49.393 | −4.652 | 61.292 | 1.00 | 19.54 | B |
| ATOM | 5010 | N | PRO | 338 | 50.643 | −3.770 | 59.618 | 1.00 | 17.27 | B |
| ATOM | 5011 | CD | PRO | 338 | 50.949 | −2.790 | 58.555 | 1.00 | 15.95 | B |
| ATOM | 5012 | CA | PRO | 338 | 51.398 | −5.005 | 59.403 | 1.00 | 15.90 | B |
| ATOM | 5013 | CB | PRO | 338 | 51.851 | −4.868 | 57.953 | 1.00 | 14.63 | B |
| ATOM | 5014 | CG | PRO | 338 | 52.158 | −3.420 | 57.858 | 1.00 | 15.30 | B |
| ATOM | 5015 | C | PRO | 338 | 52.574 | −5.124 | 60.360 | 1.00 | 15.45 | B |
| ATOM | 5016 | O | PRO | 338 | 53.206 | −6.145 | 60.420 | 1.00 | 15.18 | B |
| ATOM | 5017 | N | ALA | 339 | 52.844 | −4.053 | 61.103 | 1.00 | 16.79 | B |
| ATOM | 5018 | CA | ALA | 339 | 53.986 | −3.999 | 62.025 | 1.00 | 19.03 | B |
| ATOM | 5019 | CB | ALA | 339 | 54.296 | −2.536 | 62.409 | 1.00 | 17.80 | B |
| ATOM | 5020 | C | ALA | 339 | 53.813 | −4.824 | 63.277 | 1.00 | 19.74 | B |
| ATOM | 5021 | O | ALA | 339 | 52.727 | −4.883 | 63.824 | 1.00 | 21.39 | B |
| ATOM | 5022 | N | SER | 340 | 54.896 | −5.452 | 63.734 | 1.00 | 20.20 | B |
| ATOM | 5023 | CA | SER | 340 | 54.825 | −6.278 | 64.940 | 1.00 | 20.54 | B |
| ATOM | 5024 | CB | SER | 340 | 56.045 | −7.193 | 65.075 | 1.00 | 21.46 | B |
| ATOM | 5025 | OG | SER | 340 | 57.233 | −6.430 | 65.182 | 1.00 | 24.93 | B |
| ATOM | 5026 | C | SER | 340 | 54.727 | −5.453 | 66.208 | 1.00 | 19.22 | B |
| ATOM | 5027 | O | SER | 340 | 54.293 | −5.941 | 67.224 | 1.00 | 17.09 | B |
| ATOM | 5028 | N | LEU | 341 | 55.131 | −4.191 | 66.143 | 1.00 | 20.29 | B |
| ATOM | 5029 | CA | LEU | 341 | 55.048 | −3.345 | 67.328 | 1.00 | 21.64 | B |
| ATOM | 5030 | CB | LEU | 341 | 56.040 | −2.184 | 67.248 | 1.00 | 23.99 | B |
| ATOM | 5031 | CG | LEU | 341 | 55.610 | −0.896 | 66.546 | 1.00 | 27.23 | B |
| ATOM | 5032 | CD1 | LEU | 341 | 55.641 | 0.269 | 67.554 | 1.00 | 26.67 | B |
| ATOM | 5033 | CD2 | LEU | 341 | 56.542 | −0.630 | 65.357 | 1.00 | 28.22 | B |
| ATOM | 5034 | C | LEU | 341 | 53.629 | −2.807 | 67.502 | 1.00 | 21.40 | B |
| ATOM | 5035 | O | LEU | 341 | 53.350 | −2.053 | 68.424 | 1.00 | 21.64 | B |
| ATOM | 5036 | N | ASN | 342 | 52.736 | −3.227 | 66.613 | 1.00 | 21.16 | B |
| ATOM | 5037 | CA | ASN | 342 | 51.335 | −2.815 | 66.664 | 1.00 | 21.98 | B |
| ATOM | 5038 | CB | ASN | 342 | 50.943 | −2.165 | 65.352 | 1.00 | 20.54 | B |
| ATOM | 5039 | CG | ASN | 342 | 51.586 | −0.826 | 65.172 | 1.00 | 21.64 | B |
| ATOM | 5040 | OD1 | ASN | 342 | 51.897 | −0.423 | 64.046 | 1.00 | 19.82 | B |
| ATOM | 5041 | ND2 | ASN | 342 | 51.785 | −0.107 | 66.285 | 1.00 | 20.76 | B |
| ATOM | 5042 | C | ASN | 342 | 50.415 | −4.011 | 66.892 | 1.00 | 22.33 | B |
| ATOM | 5043 | O | ASN | 342 | 49.201 | −3.909 | 66.761 | 1.00 | 22.21 | B |
| ATOM | 5044 | N | LEU | 343 | 51.023 | −5.135 | 67.254 | 1.00 | 23.56 | B |
| ATOM | 5045 | CA | LEU | 343 | 50.334 | −6.406 | 67.488 | 1.00 | 24.35 | B |
| ATOM | 5046 | CB | LEU | 343 | 51.360 | −7.435 | 67.992 | 1.00 | 25.91 | B |
| ATOM | 5047 | CG | LEU | 343 | 50.986 | −8.890 | 68.316 | 1.00 | 28.30 | B |
| ATOM | 5048 | CD1 | LEU | 343 | 50.524 | −8.995 | 69.761 | 1.00 | 29.51 | B |
| ATOM | 5049 | CD2 | LEU | 343 | 49.930 | −9.392 | 67.334 | 1.00 | 28.29 | B |
| ATOM | 5050 | C | LEU | 343 | 49.119 | −6.347 | 68.412 | 1.00 | 22.80 | B |
| ATOM | 5051 | O | LEU | 343 | 48.024 | −6.756 | 68.045 | 1.00 | 21.40 | B |
| ATOM | 5052 | N | GLU | 344 | 49.305 | −5.831 | 69.614 | 1.00 | 23.08 | B |
| ATOM | 5053 | CA | GLU | 344 | 48.189 | −5.745 | 70.545 | 1.00 | 22.34 | B |
| ATOM | 5054 | CB | GLU | 344 | 48.628 | −5.122 | 71.861 | 1.00 | 24.68 | B |
| ATOM | 5055 | CG | GLU | 344 | 47.491 | −4.875 | 72.821 | 1.00 | 30.10 | B |
| ATOM | 5056 | CD | GLU | 344 | 47.965 | −4.715 | 74.263 | 1.00 | 34.59 | B |
| ATOM | 5057 | OE1 | GLU | 344 | 48.866 | −3.886 | 74.538 | 1.00 | 36.85 | B |
| ATOM | 5058 | OE2 | GLU | 344 | 47.422 | −5.428 | 75.134 | 1.00 | 36.33 | B |
| ATOM | 5059 | C | GLU | 344 | 47.002 | −4.960 | 70.002 | 1.00 | 19.86 | B |
| ATOM | 5060 | O | GLU | 344 | 45.894 | −5.425 | 70.097 | 1.00 | 20.25 | B |
| ATOM | 5061 | N | GLU | 345 | 47.241 | −3.770 | 69.452 | 1.00 | 17.13 | B |
| ATOM | 5062 | CA | GLU | 345 | 46.141 | −2.974 | 68.907 | 1.00 | 16.35 | B |
| ATOM | 5063 | CB | GLU | 345 | 46.585 | −1.527 | 68.589 | 1.00 | 15.68 | B |
| ATOM | 5064 | CG | GLU | 345 | 46.803 | −0.645 | 69.824 | 1.00 | 13.57 | B |
| ATOM | 5065 | CD | GLU | 345 | 45.528 | −0.391 | 70.618 | 1.00 | 13.00 | B |
| ATOM | 5066 | OE1 | GLU | 345 | 45.623 | 0.062 | 71.768 | 1.00 | 14.32 | B |
| ATOM | 5067 | OE2 | GLU | 345 | 44.419 | −0.628 | 70.111 | 1.00 | 13.44 | B |
| ATOM | 5068 | C | GLU | 345 | 45.528 | −3.626 | 67.659 | 1.00 | 14.78 | B |
| ATOM | 5069 | O | GLU | 345 | 44.326 | −3.544 | 67.442 | 1.00 | 14.79 | B |
| ATOM | 5070 | N | THR | 346 | 46.350 | −4.284 | 66.846 | 1.00 | 14.54 | B |
| ATOM | 5071 | CA | THR | 346 | 45.863 | −4.959 | 65.641 | 1.00 | 14.71 | B |
| ATOM | 5072 | CB | THR | 346 | 47.046 | −5.572 | 64.839 | 1.00 | 15.75 | B |
| ATOM | 5073 | OG1 | THR | 346 | 47.870 | −4.523 | 64.301 | 1.00 | 19.38 | B |
| ATOM | 5074 | CG2 | THR | 346 | 46.520 | −6.467 | 63.721 | 1.00 | 15.93 | B |
| ATOM | 5075 | C | THR | 346 | 44.888 | −6.075 | 66.057 | 1.00 | 14.75 | B |
| ATOM | 5076 | O | THR | 346 | 43.863 | −6.320 | 65.403 | 1.00 | 12.97 | B |
| ATOM | 5077 | N | LEU | 347 | 45.210 | −6.741 | 67.165 | 1.00 | 15.11 | B |
| ATOM | 5078 | CA | LEU | 347 | 44.371 | −7.819 | 67.693 | 1.00 | 14.94 | B |
| ATOM | 5079 | CB | LEU | 347 | 45.080 | −8.601 | 68.797 | 1.00 | 13.17 | B |
| ATOM | 5080 | CG | LEU | 347 | 46.253 | −9.465 | 68.342 | 1.00 | 12.75 | B |
| ATOM | 5081 | CD1 | LEU | 347 | 46.845 | −10.156 | 69.559 | 1.00 | 9.82 | B |
| ATOM | 5082 | CD2 | LEU | 347 | 45.781 | −10.459 | 67.281 | 1.00 | 10.19 | B |
| ATOM | 5083 | C | LEU | 347 | 43.074 | −7.289 | 68.277 | 1.00 | 14.55 | B |
| ATOM | 5084 | O | LEU | 347 | 42.039 | −7.935 | 68.196 | 1.00 | 16.59 | B |
| ATOM | 5085 | N | SER | 348 | 43.127 | −6.107 | 68.872 | 1.00 | 14.94 | B |
| ATOM | 5086 | CA | SER | 348 | 41.917 | −5.534 | 69.425 | 1.00 | 12.88 | B |
| ATOM | 5087 | CB | SER | 348 | 42.236 | −4.288 | 70.204 | 1.00 | 11.62 | B |
| ATOM | 5088 | OG | SER | 348 | 42.841 | −4.656 | 71.416 | 1.00 | 18.29 | B |
| ATOM | 5089 | C | SER | 348 | 40.974 | −5.180 | 68.303 | 1.00 | 12.87 | B |
| ATOM | 5090 | O | SER | 348 | 39.809 | −5.505 | 68.355 | 1.00 | 12.88 | B |
| ATOM | 5091 | N | THR | 349 | 41.494 | −4.518 | 67.281 | 1.00 | 12.34 | B |
| ATOM | 5092 | CA | THR | 349 | 40.672 | −4.121 | 66.151 | 1.00 | 14.07 | B |
| ATOM | 5093 | CB | THR | 349 | 41.515 | −3.400 | 65.081 | 1.00 | 14.87 | B |
| ATOM | 5094 | OG1 | THR | 349 | 41.887 | −2.096 | 65.535 | 1.00 | 17.94 | B |
| ATOM | 5095 | CG2 | THR | 349 | 40.738 | −3.238 | 63.828 | 1.00 | 15.48 | B |
| ATOM | 5096 | C | THR | 349 | 39.992 | −5.321 | 65.493 | 1.00 | 16.16 | B |
| ATOM | 5097 | O | THR | 349 | 38.770 | −5.325 | 65.282 | 1.00 | 15.82 | B |
| ATOM | 5098 | N | LEU | 350 | 40.777 | −6.339 | 65.157 | 1.00 | 15.00 | B |
| ATOM | 5099 | CA | LEU | 350 | 40.226 | −7.518 | 64.508 | 1.00 | 15.08 | B |
| ATOM | 5100 | CB | LEU | 350 | 41.352 | −8.496 | 64.206 | 1.00 | 14.08 | B |
| ATOM | 5101 | CG | LEU | 350 | 41.963 | −8.503 | 62.812 | 1.00 | 10.95 | B |
| ATOM | 5102 | CD1 | LEU | 350 | 42.004 | −7.143 | 62.214 | 1.00 | 10.81 | B |
| ATOM | 5103 | CD2 | LEU | 350 | 43.347 | −9.038 | 62.947 | 1.00 | 11.99 | B |
| ATOM | 5104 | C | LEU | 350 | 39.162 | −8.172 | 65.367 | 1.00 | 16.48 | B |
| ATOM | 5105 | O | LEU | 350 | 38.132 | −8.595 | 64.876 | 1.00 | 17.28 | B |
| ATOM | 5106 | N | GLU | 351 | 39.443 | −8.254 | 66.658 | 1.00 | 18.22 | B |
| ATOM | 5107 | CA | GLU | 351 | 38.514 | −8.842 | 67.609 | 1.00 | 19.87 | B |
| ATOM | 5108 | CB | GLU | 351 | 39.144 | −8.846 | 69.003 | 1.00 | 21.84 | B |
| ATOM | 5109 | CG | GLU | 351 | 38.494 | −9.791 | 69.965 | 1.00 | 26.42 | B |
| ATOM | 5110 | CD | GLU | 351 | 38.420 | −11.196 | 69.403 | 1.00 | 30.21 | B |
| ATOM | 5111 | OE1 | GLU | 351 | 39.481 | −11.771 | 69.051 | 1.00 | 29.53 | B |
| ATOM | 5112 | OE2 | GLU | 351 | 37.289 | −11.724 | 69.309 | 1.00 | 32.89 | B |
| ATOM | 5113 | C | GLU | 351 | 37.217 | −8.024 | 67.646 | 1.00 | 19.18 | B |
| ATOM | 5114 | O | GLU | 351 | 36.126 | −8.569 | 67.714 | 1.00 | 19.57 | B |
| ATOM | 5115 | N | TYR | 352 | 37.368 | −6.703 | 67.603 | 1.00 | 18.87 | B |
| ATOM | 5116 | CA | TYR | 352 | 36.258 | −5.756 | 67.646 | 1.00 | 17.30 | B |
| ATOM | 5117 | CB | TYR | 352 | 36.816 | −4.348 | 67.891 | 1.00 | 14.25 | B |
| ATOM | 5118 | CG | TYR | 352 | 35.794 | −3.239 | 68.039 | 1.00 | 11.72 | B |
| ATOM | 5119 | CD1 | TYR | 352 | 35.105 | −2.729 | 66.933 | 1.00 | 11.26 | B |
| ATOM | 5120 | CE1 | TYR | 352 | 34.220 | −1.649 | 67.067 | 1.00 | 11.17 | B |
| ATOM | 5121 | CD2 | TYR | 352 | 35.570 | −2.654 | 69.282 | 1.00 | 10.15 | B |
| ATOM | 5122 | CE2 | TYR | 352 | 34.699 | −1.584 | 69.433 | 1.00 | 9.37 | B |
| ATOM | 5123 | CZ | TYR | 352 | 34.024 | −1.078 | 68.322 | 1.00 | 11.62 | B |
| ATOM | 5124 | OH | TYR | 352 | 33.175 | 0.010 | 68.445 | 1.00 | 14.22 | B |
| ATOM | 5125 | C | TYR | 352 | 35.442 | −5.814 | 66.362 | 1.00 | 18.80 | B |
| ATOM | 5126 | O | TYR | 352 | 34.217 | −5.852 | 66.407 | 1.00 | 19.93 | B |
| ATOM | 5127 | N | ALA | 353 | 36.115 | −5.822 | 65.216 | 1.00 | 18.33 | B |
| ATOM | 5128 | CA | ALA | 353 | 35.406 | −5.891 | 63.951 | 1.00 | 17.31 | B |
| ATOM | 5129 | CB | ALA | 353 | 36.359 | −5.698 | 62.821 | 1.00 | 16.39 | B |
| ATOM | 5130 | C | ALA | 353 | 34.680 | −7.221 | 63.785 | 1.00 | 18.36 | B |
| ATOM | 5131 | O | ALA | 353 | 33.542 | −7.249 | 63.365 | 1.00 | 18.10 | B |
| ATOM | 5132 | N | HIS | 354 | 35.354 | −8.319 | 64.119 | 1.00 | 19.39 | B |
| ATOM | 5133 | CA | HIS | 354 | 34.779 | −9.661 | 63.994 | 1.00 | 20.34 | B |
| ATOM | 5134 | CB | HIS | 354 | 35.761 | −10.712 | 64.509 | 1.00 | 22.75 | B |
| ATOM | 5135 | CG | HIS | 354 | 35.302 | −12.121 | 64.294 | 1.00 | 25.34 | B |
| ATOM | 5136 | CD2 | HIS | 354 | 34.797 | −13.031 | 65.156 | 1.00 | 25.57 | B |
| ATOM | 5137 | ND1 | HIS | 354 | 35.311 | −12.725 | 63.053 | 1.00 | 25.77 | B |
| ATOM | 5138 | CE1 | HIS | 354 | 34.831 | −13.948 | 63.164 | 1.00 | 26.03 | B |
| ATOM | 5139 | NE2 | HIS | 354 | 34.511 | −14.162 | 64.427 | 1.00 | 26.67 | B |
| ATOM | 5140 | C | HIS | 354 | 33.486 | −9.811 | 64.796 | 1.00 | 20.23 | B |
| ATOM | 5141 | O | HIS | 354 | 32.512 | −10.417 | 64.352 | 1.00 | 18.53 | B |
| ATOM | 5142 | N | ARG | 355 | 33.505 | −9.255 | 65.995 | 1.00 | 20.24 | B |
| ATOM | 5143 | CA | ARG | 355 | 32.370 | −9.285 | 66.891 | 1.00 | 20.90 | B |
| ATOM | 5144 | CB | ARG | 355 | 32.823 | −8.721 | 68.239 | 1.00 | 20.70 | B |
| ATOM | 5145 | CG | ARG | 355 | 31.789 | −8.672 | 69.339 | 1.00 | 21.77 | B |
| ATOM | 5146 | CD | ARG | 355 | 32.433 | −8.121 | 70.598 | 1.00 | 22.76 | B |
| ATOM | 5147 | NE | ARG | 355 | 31.461 | −7.943 | 71.673 | 1.00 | 27.66 | B |
| ATOM | 5148 | CZ | ARG | 355 | 30.820 | −8.942 | 72.281 | 1.00 | 31.26 | B |
| ATOM | 5149 | NH1 | ARG | 355 | 31.042 | −10.206 | 71.921 | 1.00 | 31.17 | B |
| ATOM | 5150 | NH2 | ARG | 355 | 29.965 | −8.679 | 73.262 | 1.00 | 31.12 | B |
| ATOM | 5151 | C | ARG | 355 | 31.177 | −8.513 | 66.305 | 1.00 | 21.80 | B |
| ATOM | 5152 | O | ARG | 355 | 30.040 | −8.932 | 66.453 | 1.00 | 23.53 | B |
| ATOM | 5153 | N | ALA | 356 | 31.442 | −7.394 | 65.634 | 1.00 | 21.31 | B |
| ATOM | 5154 | CA | ALA | 356 | 30.375 | −6.586 | 65.049 | 1.00 | 20.41 | B |
| ATOM | 5155 | CB | ALA | 356 | 30.924 | −5.282 | 64.583 | 1.00 | 20.58 | B |
| ATOM | 5156 | C | ALA | 356 | 29.618 | −7.256 | 63.902 | 1.00 | 20.99 | B |
| ATOM | 5157 | O | ALA | 356 | 28.531 | −6.796 | 63.543 | 1.00 | 19.69 | B |
| ATOM | 5158 | N | LYS | 357 | 30.195 | −8.328 | 63.340 | 1.00 | 22.58 | B |
| ATOM | 5159 | CA | LYS | 357 | 29.590 | −9.081 | 62.225 | 1.00 | 22.82 | B |
| ATOM | 5160 | CB | LYS | 357 | 30.347 | −10.371 | 61.911 | 1.00 | 23.14 | B |
| ATOM | 5161 | CG | LYS | 357 | 31.767 | −10.194 | 61.443 | 1.00 | 25.46 | B |
| ATOM | 5162 | CD | LYS | 357 | 31.897 | −10.597 | 59.983 | 1.00 | 27.85 | B |
| ATOM | 5163 | CE | LYS | 357 | 31.660 | −12.104 | 59.763 | 1.00 | 27.26 | B |
| ATOM | 5164 | NZ | LYS | 357 | 32.648 | −12.966 | 60.485 | 1.00 | 27.32 | B |
| ATOM | 5165 | C | LYS | 357 | 28.198 | −9.551 | 62.594 | 1.00 | 23.74 | B |
| ATOM | 5166 | O | LYS | 357 | 27.315 | −9.635 | 61.755 | 1.00 | 22.43 | B |
| ATOM | 5167 | N | ASN | 358 | 28.016 | −9.845 | 63.876 | 1.00 | 25.58 | B |
| ATOM | 5168 | CA | ASN | 358 | 26.730 | −10.306 | 64.388 | 1.00 | 28.23 | B |
| ATOM | 5169 | CB | ASN | 358 | 26.914 | −10.928 | 65.766 | 1.00 | 28.39 | B |
| ATOM | 5170 | CG | ASN | 358 | 27.852 | −12.105 | 65.742 | 1.00 | 29.97 | B |
| ATOM | 5171 | OD1 | ASN | 358 | 28.203 | −12.649 | 66.778 | 1.00 | 31.69 | B |
| ATOM | 5172 | ND2 | ASN | 358 | 28.267 | −12.506 | 64.551 | 1.00 | 29.57 | B |
| ATOM | 5173 | C | ASN | 358 | 25.606 | −9.270 | 64.476 | 1.00 | 30.00 | B |
| ATOM | 5174 | O | ASN | 358 | 24.487 | −9.619 | 64.845 | 1.00 | 30.93 | B |
| ATOM | 5175 | N | ILE | 359 | 25.892 | −8.011 | 64.152 | 1.00 | 31.11 | B |
| ATOM | 5176 | CA | ILE | 359 | 24.855 | −6.986 | 64.176 | 1.00 | 32.09 | B |
| ATOM | 5177 | CB | ILE | 359 | 25.465 | −5.604 | 64.142 | 1.00 | 31.91 | B |
| ATOM | 5178 | CG2 | ILE | 359 | 24.367 | −4.569 | 64.136 | 1.00 | 30.39 | B |
| ATOM | 5179 | CG1 | ILE | 359 | 26.375 | −5.433 | 65.361 | 1.00 | 32.12 | B |
| ATOM | 5180 | CD1 | ILE | 359 | 27.169 | −4.134 | 65.382 | 1.00 | 34.29 | B |
| ATOM | 5181 | C | ILE | 359 | 23.903 | −7.152 | 62.984 | 1.00 | 33.89 | B |
| ATOM | 5182 | O | ILE | 359 | 24.326 | −7.355 | 61.843 | 1.00 | 32.83 | B |
| ATOM | 5183 | N | LEU | 360 | 22.605 | −7.080 | 63.256 | 1.00 | 36.27 | B |
| ATOM | 5184 | CA | LEU | 360 | 21.597 | −7.249 | 62.211 | 1.00 | 39.23 | B |
| ATOM | 5185 | CB | LEU | 360 | 20.630 | −8.381 | 62.583 | 1.00 | 42.29 | B |
| ATOM | 5186 | CG | LEU | 360 | 19.497 | −8.742 | 61.609 | 1.00 | 44.94 | B |
| ATOM | 5187 | CD1 | LEU | 360 | 20.073 | −9.122 | 60.240 | 1.00 | 44.70 | B |
| ATOM | 5188 | CD2 | LEU | 360 | 18.676 | −9.901 | 62.188 | 1.00 | 45.24 | B |
| ATOM | 5189 | C | LEU | 360 | 20.800 | −5.970 | 62.028 | 1.00 | 39.70 | B |
| ATOM | 5190 | O | LEU | 360 | 20.286 | −5.429 | 62.994 | 1.00 | 39.55 | B |
| ATOM | 5191 | N | ASN | 361 | 20.710 | −5.509 | 60.777 | 1.00 | 40.33 | B |
| ATOM | 5192 | CA | ASN | 361 | 19.989 | −4.286 | 60.413 | 1.00 | 39.80 | B |
| ATOM | 5193 | CB | ASN | 361 | 20.865 | −3.358 | 59.573 | 1.00 | 40.62 | B |
| ATOM | 5194 | CG | ASN | 361 | 22.050 | −2.798 | 60.350 | 1.00 | 41.69 | B |
| ATOM | 5195 | OD1 | ASN | 361 | 22.893 | −2.087 | 59.792 | 1.00 | 41.21 | B |
| ATOM | 5196 | ND2 | ASN | 361 | 22.119 | −3.109 | 61.633 | 1.00 | 41.78 | B |
| ATOM | 5197 | C | ASN | 361 | 18.748 | −4.575 | 59.575 | 1.00 | 40.40 | B |
| ATOM | 5198 | O | ASN | 361 | 18.630 | −5.637 | 58.974 | 1.00 | 41.33 | B |
| ATOM | 5199 | N | LYS | 362 | 17.838 | −3.604 | 59.535 | 1.00 | 40.64 | B |
| ATOM | 5200 | CA | LYS | 362 | 16.572 | −3.687 | 58.795 | 1.00 | 40.39 | B |
| ATOM | 5201 | CB | LYS | 362 | 16.811 | −3.781 | 57.283 | 1.00 | 38.42 | B |
| ATOM | 5202 | CG | LYS | 362 | 17.283 | −2.481 | 56.664 | 1.00 | 37.04 | B |
| ATOM | 5203 | CD | LYS | 362 | 17.312 | −2.553 | 55.151 | 1.00 | 35.58 | B |
| ATOM | 5204 | CE | LYS | 362 | 15.915 | −2.479 | 54.570 | 1.00 | 35.06 | B |
| ATOM | 5205 | NZ | LYS | 362 | 15.248 | −1.182 | 54.828 | 1.00 | 33.80 | B |
| ATOM | 5206 | C | LYS | 362 | 15.654 | −4.833 | 59.222 | 1.00 | 40.02 | B |
| ATOM | 5207 | O | LYS | 362 | 15.341 | −5.705 | 58.378 | 1.00 | 41.01 | B |
| ATOM | 5208 | OXT | LYS | 362 | 15.244 | −4.848 | 60.404 | 1.00 | 38.46 | B |
| ATOM | 5209 | MG | MG | 2602 | 43.447 | 10.556 | 59.883 | 1.00 | 1.46 | |
| ATOM | 5238 | PB | ADP | 2600 | 44.598 | 7.110 | 60.307 | 1.00 | 12.39 | ADP |
| ATOM | 5239 | O1B | ADP | 2600 | 45.185 | 7.724 | 61.540 | 1.00 | 6.06 | ADP |
| ATOM | 5240 | O2B | ADP | 2600 | 44.098 | 5.627 | 60.595 | 1.00 | 9.47 | ADP |
| ATOM | 5241 | O3B | ADP | 2600 | 43.494 | 7.932 | 59.799 | 1.00 | 9.32 | ADP |
| ATOM | 5242 | PA | ADP | 2600 | 45.933 | 7.683 | 57.885 | 1.00 | 15.76 | ADP |
| ATOM | 5243 | O1A | ADP | 2600 | 44.910 | 7.319 | 56.926 | 1.00 | 19.46 | ADP |
| ATOM | 5244 | O2A | ADP | 2600 | 45.886 | 9.129 | 58.130 | 1.00 | 18.59 | ADP |
| ATOM | 5245 | O3A | ADP | 2600 | 45.669 | 6.908 | 59.185 | 1.00 | 14.04 | ADP |
| ATOM | 5246 | O5* | ADP | 2600 | 47.412 | 7.404 | 57.328 | 1.00 | 19.34 | ADP |
| ATOM | 5247 | C5* | ADP | 2600 | 48.489 | 6.585 | 57.824 | 1.00 | 22.53 | ADP |
| ATOM | 5248 | C4* | ADP | 2600 | 49.691 | 6.801 | 56.820 | 1.00 | 24.49 | ADP |
| ATOM | 5249 | O4* | ADP | 2600 | 49.780 | 5.604 | 56.098 | 1.00 | 26.34 | ADP |
| ATOM | 5250 | C3* | ADP | 2600 | 49.504 | 7.928 | 55.757 | 1.00 | 24.13 | ADP |
| ATOM | 5251 | O3* | ADP | 2600 | 50.670 | 8.755 | 55.611 | 1.00 | 26.52 | ADP |
| ATOM | 5252 | C2* | ADP | 2600 | 49.154 | 7.243 | 54.456 | 1.00 | 25.11 | ADP |
| ATOM | 5253 | O2* | ADP | 2600 | 49.698 | 7.905 | 53.303 | 1.00 | 27.28 | ADP |
| ATOM | 5254 | C1* | ADP | 2600 | 49.652 | 5.829 | 54.676 | 1.00 | 26.94 | ADP |
| ATOM | 5255 | N9 | ADP | 2600 | 48.736 | 4.765 | 54.191 | 1.00 | 27.64 | ADP |
| ATOM | 5256 | C8 | ADP | 2600 | 47.767 | 4.193 | 54.941 | 1.00 | 26.96 | ADP |
| ATOM | 5257 | N7 | ADP | 2600 | 47.150 | 3.292 | 54.228 | 1.00 | 29.21 | ADP |
| ATOM | 5258 | C5 | ADP | 2600 | 47.690 | 3.269 | 53.027 | 1.00 | 29.55 | ADP |
| ATOM | 5259 | C6 | ADP | 2600 | 47.466 | 2.525 | 51.857 | 1.00 | 29.68 | ADP |
| ATOM | 5260 | N6 | ADP | 2600 | 46.495 | 1.606 | 51.861 | 1.00 | 29.43 | ADP |
| ATOM | 5261 | N1 | ADP | 2600 | 48.250 | 2.751 | 50.704 | 1.00 | 30.06 | ADP |
| ATOM | 5262 | C2 | ADP | 2600 | 49.252 | 3.696 | 50.678 | 1.00 | 29.27 | ADP |
| ATOM | 5263 | N3 | ADP | 2600 | 49.466 | 4.411 | 51.827 | 1.00 | 29.94 | ADP |
| ATOM | 5264 | C4 | ADP | 2600 | 48.711 | 4.230 | 52.991 | 1.00 | 28.23 | ADP |
| ATOM | 5291 | C1 | 4-2A | 1 | 42.197 | 14.937 | 49.097 | 1.00 | 25.59 | 4-2A |
| ATOM | 5292 | C2 | 4-2A | 1 | 41.920 | 14.433 | 47.714 | 1.00 | 25.74 | 4-2A |
| ATOM | 5293 | C3 | 4-2A | 1 | 41.044 | 15.120 | 46.829 | 1.00 | 26.03 | 4-2A |
| ATOM | 5294 | C4 | 4-2A | 1 | 40.929 | 14.774 | 45.500 | 1.00 | 26.67 | 4-2A |
| ATOM | 5295 | C5 | 4-2A | 1 | 41.663 | 13.715 | 44.991 | 1.00 | 25.62 | 4-2A |
| ATOM | 5296 | C6 | 4-2A | 1 | 42.514 | 12.931 | 45.817 | 1.00 | 25.53 | 4-2A |
| ATOM | 5297 | C7 | 4-2A | 1 | 42.617 | 13.291 | 47.201 | 1.00 | 25.82 | 4-2A |
| ATOM | 5298 | O12 | 4-2A | 1 | 43.246 | 11.914 | 45.291 | 1.00 | 25.59 | 4-2A |
| ATOM | 5299 | C14 | 4-2A | 1 | 40.974 | 14.917 | 49.926 | 1.00 | 26.54 | 4-2A |
| ATOM | 5300 | C15 | 4-2A | 1 | 40.461 | 16.085 | 50.528 | 1.00 | 26.66 | 4-2A |
| ATOM | 5301 | C16 | 4-2A | 1 | 41.255 | 17.420 | 50.551 | 1.00 | 26.17 | 4-2A |
| ATOM | 5302 | C17 | 4-2A | 1 | 42.265 | 17.452 | 49.404 | 1.00 | 26.31 | 4-2A |
| ATOM | 5303 | N18 | 4-2A | 1 | 42.979 | 16.179 | 49.355 | 1.00 | 26.30 | 4-2A |
| ATOM | 5304 | C22 | 4-2A | 1 | 43.422 | 18.425 | 49.565 | 1.00 | 25.84 | 4-2A |
| ATOM | 5305 | N23 | 4-2A | 1 | 44.551 | 17.713 | 49.505 | 1.00 | 25.90 | 4-2A |
| ATOM | 5306 | C24 | 4-2A | 1 | 44.289 | 16.370 | 49.394 | 1.00 | 26.52 | 4-2A |
| ATOM | 5307 | N26 | 4-2A | 1 | 40.109 | 13.877 | 50.027 | 1.00 | 26.97 | 4-2A |
| ATOM | 5308 | C27 | 4-2A | 1 | 38.991 | 14.325 | 50.732 | 1.00 | 26.51 | 4-2A |
| ATOM | 5309 | C28 | 4-2A | 1 | 39.211 | 15.740 | 51.093 | 1.00 | 27.62 | 4-2A |
| ATOM | 5310 | C29 | 4-2A | 1 | 37.745 | 13.725 | 51.140 | 1.00 | 26.04 | 4-2A |
| ATOM | 5311 | C30 | 4-2A | 1 | 36.783 | 14.431 | 51.909 | 1.00 | 26.80 | 4-2A |
| ATOM | 5312 | C31 | 4-2A | 1 | 37.035 | 15.782 | 52.312 | 1.00 | 27.44 | 4-2A |
| ATOM | 5313 | C32 | 4-2A | 1 | 38.217 | 16.439 | 51.892 | 1.00 | 27.46 | 4-2A |
| ATOM | 5314 | O37 | 4-2A | 1 | 43.236 | 19.647 | 49.683 | 1.00 | 24.48 | 4-2A |
| ATOM | 5315 | O38 | 4-2A | 1 | 45.096 | 15.436 | 49.375 | 1.00 | 27.32 | 4-2A |
| ATOM | 5316 | C39 | 4-2A | 1 | 45.831 | 18.372 | 49.744 | 1.00 | 25.80 | 4-2A |
| END | | | | | | | | | | |

TABLE 5
REMARK 1 kin_16dpb molecule B
REMARK r = 0.2114 free_r = 0.2639
REMARK rmsd bonds = 0.006712 rmsd angles = 1.32262
REMARK B rmsd for bonded mainchain atoms = 1.570 target = 1.5
REMARK B rmsd for bonded sidechain atoms = 2.570 target = 2.0
REMARK B rmsd for angle mainchain atoms = 2.729 target = 2.0
REMARK B rmsd for angle sidechain atoms = 3.936 target = 2.5
REMARK sg = P2(1)2(1)2(1) a = 69.48 b = 79.54 c = 158.98 alpha = 90. beta = 90.
gamma = 90.
REMARK reflection file = k2a.cv
REMARK B-correction resolution: 6.0-2.5
REMARK FILENAME = “kin_16dpb.pdb”

| | | | | | | | | | | |
|---|---|---|---|---|---|---|---|---|---|---|
| ATOM | 788 | N | GLU | 116 | 39.151 | 9.227 | 52.663 | 1.00 | 8.87 | B |
| ATOM | 789 | CA | GLU | 116 | 39.430 | 10.450 | 51.915 | 1.00 | 8.17 | B |
| ATOM | 790 | CB | GLU | 116 | 39.921 | 11.534 | 52.868 | 1.00 | 8.92 | B |
| ATOM | 791 | CG | GLU | 116 | 38.920 | 11.894 | 53.939 | 1.00 | 12.15 | B |
| ATOM | 792 | CD | GLU | 116 | 39.349 | 13.091 | 54.738 | 1.00 | 15.35 | B |
| ATOM | 793 | OE1 | GLU | 116 | 40.362 | 13.717 | 54.354 | 1.00 | 17.99 | B |
| ATOM | 794 | OE2 | GLU | 116 | 38.678 | 13.410 | 55.737 | 1.00 | 15.94 | B |
| ATOM | 795 | C | GLU | 116 | 40.426 | 10.321 | 50.784 | 1.00 | 8.20 | B |
| ATOM | 796 | O | GLU | 116 | 40.163 | 10.736 | 49.657 | 1.00 | 4.89 | B |
| ATOM | 797 | N | GLY | 117 | 41.577 | 9.744 | 51.097 | 1.00 | 9.09 | B |
| ATOM | 798 | CA | GLY | 117 | 42.619 | 9.608 | 50.104 | 1.00 | 10.26 | B |
| ATOM | 799 | C | GLY | 117 | 43.531 | 10.819 | 50.183 | 1.00 | 11.18 | B |
| ATOM | 800 | O | GLY | 117 | 43.289 | 11.751 | 50.951 | 1.00 | 10.98 | B |
| ATOM | 801 | N | GLU | 118 | 44.590 | 10.813 | 49.389 | 1.00 | 13.18 | B |
| ATOM | 802 | CA | GLU | 118 | 45.531 | 11.922 | 49.386 | 1.00 | 14.36 | B |
| ATOM | 803 | CB | GLU | 118 | 46.849 | 11.498 | 50.043 | 1.00 | 15.18 | B |
| ATOM | 804 | CG | GLU | 118 | 46.685 | 10.756 | 51.363 | 1.00 | 21.23 | B |
| ATOM | 805 | CD | GLU | 118 | 48.014 | 10.310 | 51.970 | 1.00 | 24.46 | B |
| ATOM | 806 | OE1 | GLU | 118 | 48.894 | 9.845 | 51.215 | 1.00 | 27.49 | B |
| ATOM | 807 | OE2 | GLU | 118 | 48.177 | 10.413 | 53.205 | 1.00 | 26.10 | B |
| ATOM | 808 | C | GLU | 118 | 45.770 | 12.281 | 47.933 | 1.00 | 13.80 | B |
| ATOM | 809 | O | GLU | 118 | 45.126 | 11.734 | 47.041 | 1.00 | 14.44 | B |
| ATOM | 810 | N | ARG | 119 | 46.689 | 13.201 | 47.685 | 1.00 | 13.24 | B |
| ATOM | 811 | CA | ARG | 119 | 46.984 | 13.568 | 46.315 | 1.00 | 14.66 | B |
| ATOM | 812 | CB | ARG | 119 | 47.120 | 15.088 | 46.167 | 1.00 | 12.36 | B |
| ATOM | 813 | CG | ARG | 119 | 45.879 | 15.905 | 46.518 | 1.00 | 11.10 | B |
| ATOM | 814 | CD | ARG | 119 | 44.628 | 15.371 | 45.842 | 1.00 | 12.06 | B |
| ATOM | 815 | NE | ARG | 119 | 44.829 | 15.087 | 44.422 | 1.00 | 13.81 | B |
| ATOM | 816 | CZ | ARG | 119 | 44.750 | 15.992 | 43.451 | 1.00 | 14.81 | B |
| ATOM | 817 | NH1 | ARG | 119 | 44.464 | 17.257 | 43.742 | 1.00 | 13.37 | B |
| ATOM | 818 | NH2 | ARG | 119 | 44.964 | 15.632 | 42.189 | 1.00 | 11.75 | B |
| ATOM | 819 | C | ARG | 119 | 48.288 | 12.911 | 45.889 | 1.00 | 16.73 | B |
| ATOM | 820 | O | ARG | 119 | 49.253 | 12.857 | 46.662 | 1.00 | 17.59 | B |
| ATOM | 879 | N | TRP | 127 | 42.371 | 15.847 | 40.233 | 1.00 | 18.06 | B |
| ATOM | 880 | CA | TRP | 127 | 41.717 | 15.171 | 41.335 | 1.00 | 16.78 | B |
| ATOM | 881 | CB | TRP | 127 | 40.912 | 16.167 | 42.178 | 1.00 | 14.46 | B |
| ATOM | 882 | CG | TRP | 127 | 39.646 | 16.618 | 41.539 | 1.00 | 10.93 | B |
| ATOM | 883 | CD2 | TRP | 127 | 38.365 | 15.996 | 41.664 | 1.00 | 8.71 | B |
| ATOM | 884 | CE2 | TRP | 127 | 37.452 | 16.770 | 40.915 | 1.00 | 9.40 | B |
| ATOM | 885 | CE3 | TRP | 127 | 37.901 | 14.857 | 42.334 | 1.00 | 7.23 | B |
| ATOM | 886 | CD1 | TRP | 127 | 39.474 | 17.709 | 40.738 | 1.00 | 10.58 | B |
| ATOM | 887 | NE1 | TRP | 127 | 38.153 | 17.810 | 40.361 | 1.00 | 8.88 | B |
| ATOM | 888 | CZ2 | TRP | 127 | 36.095 | 16.446 | 40.820 | 1.00 | 9.55 | B |
| ATOM | 889 | CZ3 | TRP | 127 | 36.545 | 14.526 | 42.242 | 1.00 | 9.73 | B |
| ATOM | 890 | CH2 | TRP | 127 | 35.659 | 15.324 | 41.488 | 1.00 | 11.69 | B |
| ATOM | 891 | C | TRP | 127 | 40.828 | 14.002 | 40.941 | 1.00 | 17.94 | B |
| ATOM | 892 | O | TRP | 127 | 40.817 | 12.978 | 41.621 | 1.00 | 18.94 | B |
| ATOM | 911 | N | ASP | 130 | 43.130 | 10.872 | 40.183 | 1.00 | 18.67 | B |
| ATOM | 912 | CA | ASP | 130 | 44.174 | 10.489 | 41.121 | 1.00 | 17.72 | B |
| ATOM | 913 | CB | ASP | 130 | 44.298 | 11.534 | 42.229 | 1.00 | 15.27 | B |
| ATOM | 914 | CG | ASP | 130 | 45.675 | 11.545 | 42.859 | 1.00 | 16.56 | B |
| ATOM | 915 | OD1 | ASP | 130 | 46.157 | 10.473 | 43.285 | 1.00 | 15.04 | B |
| ATOM | 916 | OD2 | ASP | 130 | 46.277 | 12.634 | 42.930 | 1.00 | 16.73 | B |
| ATOM | 917 | C | ASP | 130 | 43.921 | 9.115 | 41.733 | 1.00 | 16.61 | B |
| ATOM | 918 | O | ASP | 130 | 42.931 | 8.905 | 42.430 | 1.00 | 19.40 | B |
| ATOM | 926 | N | LEU | 132 | 45.069 | 7.791 | 44.240 | 1.00 | 15.09 | B |
| ATOM | 927 | CA | LEU | 132 | 45.118 | 7.772 | 45.703 | 1.00 | 13.40 | B |
| ATOM | 928 | CB | LEU | 132 | 46.379 | 8.487 | 46.227 | 1.00 | 10.29 | B |
| ATOM | 929 | CG | LEU | 132 | 47.765 | 7.870 | 45.930 | 1.00 | 14.23 | B |
| ATOM | 930 | CD1 | LEU | 132 | 48.877 | 8.709 | 46.609 | 1.00 | 8.52 | B |
| ATOM | 931 | CD2 | LEU | 132 | 47.829 | 6.414 | 46.429 | 1.00 | 11.00 | B |
| ATOM | 932 | C | LEU | 132 | 43.858 | 8.395 | 46.310 | 1.00 | 12.82 | B |
| ATOM | 933 | O | LEU | 132 | 43.719 | 8.473 | 47.534 | 1.00 | 11.90 | B |
| ATOM | 934 | N | ALA | 133 | 42.936 | 8.833 | 45.457 | 1.00 | 12.47 | B |
| ATOM | 935 | CA | ALA | 133 | 41.681 | 9.414 | 45.936 | 1.00 | 12.78 | B |
| ATOM | 936 | CB | ALA | 133 | 40.826 | 9.884 | 44.755 | 1.00 | 11.66 | B |
| ATOM | 937 | C | ALA | 133 | 40.928 | 8.356 | 46.742 | 1.00 | 13.76 | B |
| ATOM | 938 | O | ALA | 133 | 40.991 | 7.163 | 46.431 | 1.00 | 13.92 | B |
| ATOM | 939 | N | GLY | 134 | 40.217 | 8.798 | 47.776 | 1.00 | 14.68 | B |
| ATOM | 940 | CA | GLY | 134 | 39.483 | 7.870 | 48.619 | 1.00 | 13.15 | B |
| ATOM | 941 | C | GLY | 134 | 38.016 | 7.752 | 48.262 | 1.00 | 14.05 | B |
| ATOM | 942 | O | GLY | 134 | 37.574 | 8.262 | 47.228 | 1.00 | 12.84 | B |
| ATOM | 951 | N | ILE | 136 | 35.223 | 9.141 | 49.530 | 1.00 | 10.60 | B |
| ATOM | 952 | CA | ILE | 136 | 34.466 | 10.377 | 49.379 | 1.00 | 10.62 | B |
| ATOM | 953 | CB | ILE | 136 | 34.843 | 11.386 | 50.482 | 1.00 | 10.47 | B |
| ATOM | 954 | CG2 | ILE | 136 | 34.175 | 12.721 | 50.231 | 1.00 | 8.18 | B |
| ATOM | 955 | CG1 | ILE | 136 | 34.382 | 10.847 | 51.839 | 1.00 | 10.73 | B |
| ATOM | 956 | CD1 | ILE | 136 | 34.760 | 11.746 | 53.047 | 1.00 | 13.23 | B |
| ATOM | 957 | C | ILE | 136 | 34.553 | 11.030 | 47.995 | 1.00 | 11.05 | B |
| ATOM | 958 | O | ILE | 136 | 33.531 | 11.296 | 47.373 | 1.00 | 10.67 | B |
| ATOM | 959 | N | PRO | 137 | 35.765 | 11.303 | 47.492 | 1.00 | 11.64 | B |
| ATOM | 960 | CD | PRO | 137 | 37.100 | 11.313 | 48.114 | 1.00 | 11.30 | B |
| ATOM | 961 | CA | PRO | 137 | 35.793 | 11.924 | 46.162 | 1.00 | 11.06 | B |
| ATOM | 962 | CB | PRO | 137 | 37.237 | 12.410 | 46.031 | 1.00 | 10.03 | B |
| ATOM | 963 | CG | PRO | 137 | 38.002 | 11.469 | 46.911 | 1.00 | 11.65 | B |
| ATOM | 964 | C | PRO | 137 | 35.369 | 10.997 | 45.019 | 1.00 | 11.97 | B |
| ATOM | 965 | O | PRO | 137 | 34.867 | 11.455 | 43.989 | 1.00 | 11.71 | B |
| ATOM | 1145 | N | LEU | 160 | 29.446 | 18.027 | 56.397 | 1.00 | 13.49 | B |
| ATOM | 1146 | CA | LEU | 160 | 30.595 | 17.478 | 57.077 | 1.00 | 13.18 | B |
| ATOM | 1147 | CB | LEU | 160 | 31.883 | 18.025 | 56.470 | 1.00 | 14.21 | B |
| ATOM | 1148 | CG | LEU | 160 | 33.175 | 17.477 | 57.068 | 1.00 | 13.62 | B |
| ATOM | 1149 | CD1 | LEU | 160 | 33.056 | 15.961 | 57.243 | 1.00 | 13.33 | B |
| ATOM | 1150 | CD2 | LEU | 160 | 34.343 | 17.846 | 56.166 | 1.00 | 13.39 | B |
| ATOM | 1151 | C | LEU | 160 | 30.492 | 17.857 | 58.543 | 1.00 | 13.90 | B |
| ATOM | 1152 | O | LEU | 160 | 30.883 | 18.956 | 58.947 | 1.00 | 11.88 | B |
| ATOM | 1564 | N | TYR | 211 | 35.581 | 19.271 | 44.173 | 1.00 | 18.55 | B |
| ATOM | 1565 | CA | TYR | 211 | 36.924 | 19.418 | 44.731 | 1.00 | 18.51 | B |
| ATOM | 1566 | CB | TYR | 211 | 37.994 | 19.405 | 43.637 | 1.00 | 15.05 | B |
| ATOM | 1567 | CG | TYR | 211 | 39.385 | 19.255 | 44.201 | 1.00 | 14.52 | B |
| ATOM | 1568 | CD1 | TYR | 211 | 39.721 | 18.153 | 44.981 | 1.00 | 15.06 | B |
| ATOM | 1569 | CE1 | TYR | 211 | 40.989 | 18.023 | 45.540 | 1.00 | 14.43 | B |
| ATOM | 1570 | CD2 | TYR | 211 | 40.359 | 20.232 | 43.988 | 1.00 | 13.72 | B |
| ATOM | 1571 | CE2 | TYR | 211 | 41.629 | 20.112 | 44.541 | 1.00 | 12.86 | B |
| ATOM | 1572 | CZ | TYR | 211 | 41.937 | 19.003 | 45.316 | 1.00 | 13.41 | B |
| ATOM | 1573 | OH | TYR | 211 | 43.192 | 18.863 | 45.864 | 1.00 | 13.57 | B |
| ATOM | 1574 | C | TYR | 211 | 37.044 | 20.683 | 45.575 | 1.00 | 19.47 | B |
| ATOM | 1575 | O | TYR | 211 | 37.567 | 20.640 | 46.688 | 1.00 | 21.09 | B |
| ATOM | 1593 | N | LEU | 214 | 35.512 | 20.128 | 48.935 | 1.00 | 13.24 | B |
| ATOM | 1594 | CA | LEU | 214 | 36.304 | 19.274 | 49.805 | 1.00 | 13.61 | B |
| ATOM | 1595 | CB | LEU | 214 | 36.778 | 18.022 | 49.055 | 1.00 | 11.20 | B |
| ATOM | 1596 | CG | LEU | 214 | 35.695 | 17.141 | 48.423 | 1.00 | 12.16 | B |
| ATOM | 1597 | CD1 | LEU | 214 | 36.340 | 15.933 | 47.756 | 1.00 | 10.83 | B |
| ATOM | 1598 | CD2 | LEU | 214 | 34.703 | 16.686 | 49.485 | 1.00 | 11.84 | B |
| ATOM | 1599 | C | LEU | 214 | 37.503 | 20.063 | 50.332 | 1.00 | 14.64 | B |
| ATOM | 1600 | O | LEU | 214 | 37.903 | 19.885 | 51.476 | 1.00 | 16.56 | B |
| ATOM | 1601 | N | GLU | 215 | 38.065 | 20.946 | 49.506 | 1.00 | 16.42 | B |
| ATOM | 1602 | CA | GLU | 215 | 39.216 | 21.748 | 49.930 | 1.00 | 18.40 | B |
| ATOM | 1603 | CB | GLU | 215 | 39.764 | 22.595 | 48.781 | 1.00 | 18.89 | B |
| ATOM | 1604 | CG | GLU | 215 | 40.428 | 21.819 | 47.673 | 1.00 | 21.62 | B |
| ATOM | 1605 | CD | GLU | 215 | 40.989 | 22.739 | 46.598 | 1.00 | 25.34 | B |
| ATOM | 1606 | OE1 | GLU | 215 | 42.227 | 22.957 | 46.572 | 1.00 | 24.25 | B |
| ATOM | 1607 | OE2 | GLU | 215 | 40.182 | 23.256 | 45.788 | 1.00 | 24.35 | B |
| ATOM | 1608 | C | GLU | 215 | 38.856 | 22.676 | 51.077 | 1.00 | 17.37 | B |
| ATOM | 1609 | O | GLU | 215 | 39.600 | 22.779 | 52.053 | 1.00 | 17.62 | B |
| ATOM | 1619 | N | GLY | 217 | 36.574 | 22.385 | 53.343 | 1.00 | 17.13 | B |
| ATOM | 1620 | CA | GLY | 217 | 36.448 | 21.651 | 54.586 | 1.00 | 16.36 | B |
| ATOM | 1621 | C | GLY | 217 | 37.821 | 21.367 | 55.173 | 1.00 | 16.18 | B |
| ATOM | 1622 | O | GLY | 217 | 38.044 | 21.542 | 56.378 | 1.00 | 15.76 | B |
| ATOM | 1623 | N | ALA | 218 | 38.746 | 20.934 | 54.322 | 1.00 | 15.35 | B |
| ATOM | 1624 | CA | ALA | 218 | 40.105 | 20.629 | 54.763 | 1.00 | 15.51 | B |
| ATOM | 1625 | CB | ALA | 218 | 40.923 | 20.071 | 53.596 | 1.00 | 14.52 | B |
| ATOM | 1626 | C | ALA | 218 | 40.806 | 21.849 | 55.356 | 1.00 | 14.85 | B |
| ATOM | 1627 | O | ALA | 218 | 41.470 | 21.745 | 56.386 | 1.00 | 15.80 | B |
| ATOM | 1642 | N | ARG | 221 | 39.496 | 22.571 | 58.714 | 1.00 | 13.46 | B |
| ATOM | 1643 | CA | ARG | 221 | 39.917 | 21.498 | 59.606 | 1.00 | 14.10 | B |
| ATOM | 1644 | CB | ARG | 221 | 39.866 | 20.171 | 58.853 | 1.00 | 13.82 | B |
| ATOM | 1645 | CG | ARG | 221 | 39.982 | 18.949 | 59.723 | 1.00 | 18.08 | B |
| ATOM | 1646 | CD | ARG | 221 | 39.939 | 17.690 | 58.874 | 1.00 | 19.00 | B |
| ATOM | 1647 | NE | ARG | 221 | 38.585 | 17.167 | 58.725 | 1.00 | 18.62 | B |
| ATOM | 1648 | CZ | ARG | 221 | 38.226 | 16.296 | 57.788 | 1.00 | 20.44 | B |
| ATOM | 1649 | NH1 | ARG | 221 | 39.122 | 15.860 | 56.905 | 1.00 | 20.22 | B |
| ATOM | 1650 | NH2 | ARG | 221 | 36.980 | 15.839 | 57.751 | 1.00 | 16.95 | B |
| ATOM | 1651 | C | ARG | 221 | 41.331 | 21.780 | 60.137 | 1.00 | 14.31 | B |
| ATOM | 1652 | O | ARG | 221 | 41.669 | 21.408 | 61.271 | 1.00 | 14.60 | B |
| ATOM | 1777 | N | PHE | 239 | 30.844 | 12.531 | 56.963 | 1.00 | 10.36 | B |
| ATOM | 1778 | CA | PHE | 239 | 30.590 | 13.199 | 55.695 | 1.00 | 10.45 | B |
| ATOM | 1779 | CB | PHE | 239 | 31.785 | 13.041 | 54.753 | 1.00 | 10.20 | B |
| ATOM | 1780 | CG | PHE | 239 | 31.691 | 13.879 | 53.513 | 1.00 | 7.76 | B |
| ATOM | 1781 | CD1 | PHE | 239 | 30.822 | 13.533 | 52.479 | 1.00 | 7.06 | B |
| ATOM | 1782 | CD2 | PHE | 239 | 32.466 | 15.026 | 53.386 | 1.00 | 6.02 | B |
| ATOM | 1783 | CE1 | PHE | 239 | 30.729 | 14.329 | 51.327 | 1.00 | 7.31 | B |
| ATOM | 1784 | CE2 | PHE | 239 | 32.384 | 15.829 | 52.242 | 1.00 | 6.13 | B |
| ATOM | 1785 | CZ | PHE | 239 | 31.516 | 15.483 | 51.210 | 1.00 | 5.13 | B |
| ATOM | 1786 | C | PHE | 239 | 29.350 | 12.555 | 55.085 | 1.00 | 12.53 | B |
| ATOM | 1787 | O | PHE | 239 | 29.360 | 11.369 | 54.734 | 1.00 | 12.06 | B |
| ATOM | 2624 | MG | MG | 2602 | 43.714 | 10.353 | 59.884 | 1.00 | 13.44 | |
| ATOM | 2625 | PB | ADP | 2600 | 44.677 | 7.176 | 60.125 | 1.00 | 9.41 | ADP |
| ATOM | 2626 | O1B | ADP | 2600 | 45.207 | 7.814 | 61.350 | 1.00 | 10.96 | ADP |
| ATOM | 2627 | O2B | ADP | 2600 | 44.169 | 5.685 | 60.429 | 1.00 | 12.45 | ADP |
| ATOM | 2628 | O3B | ADP | 2600 | 43.584 | 7.969 | 59.545 | 1.00 | 8.39 | ADP |
| ATOM | 2629 | PA | ADP | 2600 | 46.112 | 7.788 | 57.787 | 1.00 | 12.25 | ADP |
| ATOM | 2630 | O1A | ADP | 2600 | 45.124 | 7.466 | 56.774 | 1.00 | 14.66 | ADP |
| ATOM | 2631 | O2A | ADP | 2600 | 46.054 | 9.225 | 58.059 | 1.00 | 14.40 | ADP |
| ATOM | 2632 | O3A | ADP | 2600 | 45.825 | 7.002 | 59.093 | 1.00 | 9.50 | ADP |
| ATOM | 2633 | O5* | ADP | 2600 | 47.568 | 7.490 | 57.279 | 1.00 | 16.91 | ADP |
| ATOM | 2634 | C5* | ADP | 2600 | 48.603 | 6.677 | 57.812 | 1.00 | 18.22 | ADP |
| ATOM | 2635 | C4* | ADP | 2600 | 49.807 | 6.826 | 56.807 | 1.00 | 21.00 | ADP |
| ATOM | 2636 | O4* | ADP | 2600 | 49.837 | 5.609 | 56.073 | 1.00 | 23.65 | ADP |
| ATOM | 2637 | C3* | ADP | 2600 | 49.662 | 7.936 | 55.733 | 1.00 | 20.88 | ADP |
| ATOM | 2638 | O3* | ADP | 2600 | 50.883 | 8.668 | 55.538 | 1.00 | 23.91 | ADP |
| ATOM | 2639 | C2* | ADP | 2600 | 49.227 | 7.250 | 54.452 | 1.00 | 21.72 | ADP |
| ATOM | 2640 | O2* | ADP | 2600 | 49.726 | 7.910 | 53.286 | 1.00 | 24.74 | ADP |
| ATOM | 2641 | C1* | ADP | 2600 | 49.720 | 5.835 | 54.648 | 1.00 | 22.48 | ADP |
| ATOM | 2642 | N9 | ADP | 2600 | 48.789 | 4.775 | 54.145 | 1.00 | 22.01 | ADP |
| ATOM | 2643 | C8 | ADP | 2600 | 47.775 | 4.231 | 54.861 | 1.00 | 22.26 | ADP |
| ATOM | 2644 | N7 | ADP | 2600 | 47.163 | 3.322 | 54.140 | 1.00 | 24.15 | ADP |
| ATOM | 2645 | C5 | ADP | 2600 | 47.742 | 3.257 | 52.980 | 1.00 | 24.22 | ADP |
| ATOM | 2646 | C6 | ADP | 2600 | 47.552 | 2.498 | 51.838 | 1.00 | 25.28 | ADP |
| ATOM | 2647 | N6 | ADP | 2600 | 46.577 | 1.596 | 51.801 | 1.00 | 26.60 | ADP |
| ATOM | 2648 | N1 | ADP | 2600 | 48.372 | 2.684 | 50.738 | 1.00 | 28.22 | ADP |
| ATOM | 2649 | C2 | ADP | 2600 | 49.388 | 3.599 | 50.736 | 1.00 | 27.91 | ADP |
| ATOM | 2650 | N3 | ADP | 2600 | 49.583 | 4.338 | 51.852 | 1.00 | 25.85 | ADP |
| ATOM | 2651 | C4 | ADP | 2600 | 48.803 | 4.199 | 52.972 | 1.00 | 23.75 | ADP |
| ATOM | 2879 | C1 | 5-2b | 1 | 40.179 | 14.530 | 46.990 | 1.00 | 27.45 | 5-2b |
| ATOM | 2880 | C2 | 5-2b | 1 | 41.169 | 13.921 | 47.825 | 1.00 | 31.74 | 5-2b |
| ATOM | 2881 | C3 | 5-2b | 1 | 42.197 | 13.109 | 47.246 | 1.00 | 26.68 | 5-2b |
| ATOM | 2882 | C4 | 5-2b | 1 | 42.197 | 12.949 | 45.832 | 1.00 | 25.21 | 5-2b |
| ATOM | 2883 | C5 | 5-2b | 1 | 41.213 | 13.549 | 44.997 | 1.00 | 25.57 | 5-2b |
| ATOM | 2884 | C6 | 5-2b | 1 | 40.174 | 14.358 | 45.564 | 1.00 | 26.52 | 5-2b |
| ATOM | 2885 | C7 | 5-2b | 1 | 41.159 | 14.149 | 49.287 | 1.00 | 39.17 | 5-2b |
| ATOM | 2886 | N8 | 5-2b | 1 | 40.043 | 13.644 | 50.068 | 1.00 | 32.24 | 5-2b |
| ATOM | 2887 | C9 | 5-2b | 1 | 39.077 | 14.446 | 50.550 | 1.00 | 31.10 | 5-2b |
| ATOM | 2888 | N10 | 5-2b | 1 | 39.335 | 15.753 | 50.627 | 1.00 | 35.90 | 5-2b |
| ATOM | 2889 | C11 | 5-2b | 1 | 40.586 | 16.353 | 50.204 | 1.00 | 43.34 | 5-2b |
| ATOM | 2890 | C12 | 5-2b | 1 | 41.575 | 15.550 | 49.725 | 1.00 | 51.84 | 5-2b |
| ATOM | 2891 | O13 | 5-2b | 1 | 43.103 | 12.325 | 45.318 | 1.00 | 22.27 | 5-2b |
| ATOM | 2892 | C14 | 5-2b | 1 | 43.049 | 15.950 | 49.559 | 1.00 | 69.59 | 5-2b |
| ATOM | 2893 | O15 | 5-2b | 1 | 43.510 | 17.255 | 49.536 | 1.00 | 102.78 | 5-2b |
| ATOM | 2894 | C16 | 5-2b | 1 | 44.900 | 17.802 | 49.405 | 1.00 | 94.24 | 5-2b |
| ATOM | 2895 | C17 | 5-2b | 1 | 44.910 | 19.338 | 49.209 | 1.00 | 96.86 | 5-2b |
| ATOM | 2896 | C18 | 5-2b | 1 | 40.562 | 17.864 | 50.356 | 1.00 | 41.39 | 5-2b |
| ATOM | 2897 | O19 | 5-2b | 1 | 43.806 | 15.026 | 49.427 | 1.00 | 72.75 | 5-2b |
| ATOM | 2898 | S20 | 5-2b | 1 | 37.588 | 13.867 | 51.069 | 1.00 | 18.63 | 5-2b |
| END | | | | | | | | | | |

1-74. (canceled)

75. A crystallized complex of KSP and a ligand thereof, wherein the relative structural coordinates of the amino acid residues of KSP are selected from the group set forth in one of the following:
a) Table 1 ±the root mean square deviation from the conserved backbone atoms of not more than about 2 Å;
b) Table 2 ±the root mean square deviation from the conserved backbone atoms of said amino acids of not more than about 2 Å;
c) Table 3 ±the root mean square deviation from the conserved backbone atoms of said amino acids of not more than about 2 Å; and
d) Table 4 ±the root mean square deviation from the conserved backbone atoms of said amino acids of not more than about 2 Å.

76. The crystallized complex of claim 75, wherein the relative structural coordinates of the amino acid residues are as set forth in Table 1 ±the root mean square deviation from the conserved backbone atoms of said amino acids of not more than about 0.5 Å.

77. The crystallized complex of claim 75 wherein said ligand binds said KSP at a ligand binding site comprising the KSP amino acid residues 115 (M), 116(E), 117(G), 118(E), 119(R), 127(W), 130(D), 132(L), 133(A), 134(G), 136(I), 137(P), 160(L) 211(Y), 214(L), 215(E), 217(G), 218(A), 221(R) and 239(F).

78. The crystallized complex of claim 75, wherein the relative structural coordinates of the amino acid residues are as set forth in Table 2 ±the root mean square deviation from the conserved backbone atoms of said amino acids of not more than about 0.5 Å.

79. The crystallized complex of claim 75, wherein said ligand binds said KSP at a ligand binding site comprising the KSP amino acid residues 115 (M), 116(E), 117(G), 118(E), 19(R), 127(W), 130(D), 132(L), 133(A), 134(G), 136(I), 137(P), 160(L) 211(Y), 214(L), 215(E), 217(G), 218(A), 221(R) and 239(F).

80. The crystallized complex of claim 75, wherein the relative structural coordinates of the amino acid residues are as set forth in Table 3 ±the root mean square deviation from the conserved backbone atoms of said amino acids of not more than about 0.5 Å.

81. The crystallized complex of claim 75, wherein said ligand binds said KSP at a ligand binding site comprising the KSP amino acid residues 115 (M), 116(E), 117(G), 118(E), 119(R), 127(W), 130(D), 132(L), 133(A), 134(G), 136(I), 137(P), 160(L) 211(Y), 214(L), 215(E), 217(G), 218(A), 221(R) and 239(F).

82. The crystallized complex of claim 75, wherein the relative structural coordinates of the amino acid residues are as set forth in Table 4 ±the root mean square deviation from the conserved backbone atoms of said amino acids of not more than about 0.5 Å.

83. The crystallized complex of claim 75, wherein said ligand binds said KSP at a ligand binding site comprising the KSP amino acid residues 115 (M), 116(E), 117(G), 118(E), 119(R), 127(W), 130(D), 132(L), 133(A), 134(G), 136(I), 137(P), 160(L) 211(Y), 214(L), 215(E), 217(G), 218(A), 221(R) and 239(F).

84. A ligand binding site of a KSP protein comprising the relative structural coordinates set forth in Table 5 ±the root mean square deviation from the backbone atoms of said amino acids is not more than about 2 Å.

85. The ligand binding site of a KSP protein according to claim 84 comprising the relative structural coordinates set forth in Table 5 ±the root mean square deviation from the backbone atoms of said amino acids is not more than about 0.5 Å.

86. The ligand binding site of a KSP protein according to claim 84 comprising the relative structural coordinates of the KSP amino acid residues 115 (M), 116(E), 117(G), 118(E), 119(R), 127(W), 130(D), 132(L), 133(A), 134(G), 136(I), 137(P), 160(L) 211 (Y), 214(L), 215(E), 217(G), 218(A), 221(R) and 239(F) as set forth in a table selected from a group consisting of Tables 1, 2, 3 and 4, ±the root mean square deviation from the backbone atoms of said amino acids is not more than about 2 Å.

87. A method for identifying an agent that interacts with a ligand binding site of human KSP, comprising the steps of:
(a) determining a ligand binding site of KSP from a three-dimensional model of the KSP binding site as set forth in Table 5, ±the root mean square deviation from the backbone atoms of said amino acids of not more than about 2.0 Å; and
(b) performing computer fitting analysis to identify an agent which interacts with said ligand binding site.

88. A method for identifying a potential inhibitor of KSP function, comprising the steps of:
(a) generating a three-dimensional model of KSP using the relative structural coordinates as set forth in a table selected from Tables 1, 2, 3 and 4, ±a root mean square deviation from the backbone atoms of said amino acids of not more than about 2.0 Å;
(b) employing said three-dimensional model to design or select a potential inhibitor; and
(c) synthesizing or obtaining said potential inhibitor.

89. The method according to claim 88 wherein the potential inhibitor is designed de novo.

90. The method of claim 88, further comprising the steps of:
(a) contacting said potential inhibitor with KSP in the presence of a KSP binding molecule, and
(b) determining the effect the potential inhibitor has on binding between KSP and the KSP binding molecule.

91. A machine-readable data storage medium, comprising a data storage material encoded with machine readable data which, when using a machine programmed with instructions for using said data, is capable of displaying a graphical three-dimensional representation of a molecular complex of a compound bound to the ligand binding site of human KSP, said three-dimensional representation comprising the structural coordinates of the KSP as set forth in a table selected from Tables 1-4 or a homologue of said molecular complex, wherein said homologue comprises a binding site that has a root mean square deviation from the backbone atoms of said KSP of not more than about 2.0 Å.

92. A method for determining the three-dimensional structure of a complex of KSP with a ligand thereof, which comprises obtaining X-ray diffraction data for crystals of the complex comprising the ligand bound to KSP at a ligand binding site; and utilizing said data to define the three-dimensional structure of the complex.

93. A method of identifying an inhibitor of KSP wherein the inhibitor binds to the ligand binding site according to claim 84 which comprises determining the shift in the fluorescence of an amino acid residue at position 127 of KSP, wherein said amino acid residue is tryptophan.

94. The method according to claim 93 which comprises the steps of:
(a) contacting KSP with the test compound and a nucleotide and measuring the fluorescence of the mixture at the peak emission wavelength for W127 in KSP;
(b) contacting KSP with a nucleotide and measuring the fluorescence of the mixture at the peak emission wavelength for W127 in KSP; and
(c) comparing the fluorescence of the mixture of KSP, the test compound and the nucleotide with the fluorescence of the mixture of KSP with the nucleotide alone.


SEQUENCE LISTING (1 sequence)

SEQ ID NO: 1 - protein, 368 aa, human
MASQPNSSAKKKEEKGKNIQVVVRCRPFNLAERKASAHSIVECDPVRKEVSVRTGGLADKSSRKTYTFDMVFGASTKQIDVYRSVVCPILDEVIMGYNCTIFAYGQTGTGKTFTMEGERSPNEEYTWEEDPLAGIIPRTLHQIFEKLTDNGTEFSVKVSLLEIYNEELFDLLNPSSDVSERLQMFDDPRNKRGVIIKGLEEITVHNKDEVYQILEKGAAKRTTAATLMNAYSSRSHSVFSVTIHMKETTIDGEELVKIGKLNLVDLAGSENIGRSGAVDKRAREAGNINQSLLTLGRVITALVERTPHVPYRESKLTRILQDSLGGRTRTSIIATISPASLNLEETLSTLEYAHRAKNILNKPEVNQK